(19) United States
(12) Patent Application Publication
(10) Pub. No.: US 2026/0184710 A1
(43) Pub. Date: Jul. 2, 2026

(54) CASITAS B-LINEAGE LYMPHOMA PROTOONCOGENE B (CBL-B) DEGRADING COMPOUNDS AND ASSOCIATED METHODS OF USE

(71) Applicant: Arvinas Operations, Inc., New Haven, CT (US)

(72) Inventors: Erika Marina Vieira Araujo, Woodbridge, CT (US); Albert M. Deberardinis, West Hartford, CT (US); Dana Klug, New Haven, CT (US); Jesus Rual Medina, Gilbertsville, PA (US)

(21) Appl. No.: 19/131,277

(22) PCT Filed: Nov. 20, 2023
(86) PCT No.: PCT/US2023/080571

§ 371 Date: May 20, 2025

Related U.S. Application Data
(60) Provisional application No. 63/426,850, filed on Nov. 21, 2022.
(60) Provisional application No. 63/527,701, filed on Jul. 19, 2023.

(51) Int. Cl. C07D 471/10; A61K 31/454; A61K 31/4545; A61K 31/496; A61K 31/499; A61K 31/4995; A61K 31/506; A61K 31/513; A61K 31/551; C07D 401/14; C07D 405/14; C07D 471/04; C07D 487/08; C07D 487/10; C07D 491/107; C07D 519/00

(52) U.S. Cl. CPC C07D 471/10; A61K 31/454; A61K 31/4545; A61K 31/496; A61K 31/499; A61K 31/4995; A61K 31/506; A61K 31/513; A61K 31/551; C07D 401/14; C07D 405/14; C07D 471/04; C07D 487/08; C07D 487/10; C07D 491/107; C07D 519/00

(57) ABSTRACT

Provided herein are compounds of formula (I) and salts and compositions thereof which find utility as modulators of Cbl-b in the treatment of various forms of cancer.

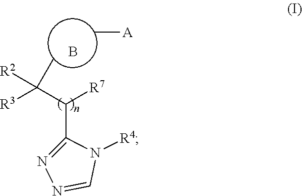

### RELATED APPLICATIONS

This application claims the benefit of priority to U.S. Provisional Application No. 63/426,850, filed Nov. 21, 2022 and U.S. Provisional Application No. 63/527,701, filed Jul. 19, 2023. The entire contents of each of the foregoing applications are incorporated herein by reference.

### BACKGROUND

Most small molecule drugs bind enzymes or receptors in tight and well-defined pockets. On the other hand, protein-protein interactions are notoriously difficult to target using small molecules due to their large contact surfaces and the shallow grooves or flat interfaces involved. E3 ubiquitin ligases (of which hundreds are known in humans) confer substrate specificity for ubiquitination, and therefore are more attractive therapeutic targets than general proteasome inhibitors due to their specificity for certain protein substrates. The development of ligands of E3 ligases has proven challenging, in part due to the fact that they must disrupt protein-protein interactions. However, recent developments have provided specific ligands that bind to these ligases. For example, since the discovery of nutlins, the first small molecule E3 ligase inhibitors, additional compounds have been reported that target E3 ligases.

Cereblon is a protein that in humans is encoded by the CRBN gene. CRBN orthologs are highly conserved from plants to humans, which underscores its physiological importance. Cereblon forms an E3 ubiquitin ligase complex with damaged DNA binding protein 1 (DDB1), Cullin-4A (CUL4A), and regulator of cullins 1 (ROC1). This complex ubiquitinates a number of other proteins. Through a mechanism which has not been completely elucidated, cereblon ubiquitination of target proteins results in increased levels of fibroblast growth factor 8 (FGF8) and fibroblast growth factor 10 (FGF10). FGF8 in turn regulates a number of developmental processes, such as limb and auditory vesicle formation. The net result is that this ubiquitin ligase complex is important for limb outgrowth in embryos. In the absence of cereblon, DDB1 forms a complex with DDB2 that functions as a DNA damage-binding protein.

An ongoing need exists in the art for effective treatments for disease associated with overexpression or aggregation of casitas B-lineage lymphoma protooncogene B (Cbl-b). Cbl-b is a negative regulator of both cytotoxic T lymphocyte (CTL) and natural killer (NK) cell activation. In T cells, Cbl-b attenuates TCR signaling by negatively regulating several downstream signaling components, enforcing the requirement for costimulation. In NK cells, Cbl-b regulates TAM receptor internalization at the plasma membrane via ubiquitylation, and this process is important in enabling inhibitory signaling via the TAM receptors. Deficiencies in active Cbl-b have been linked to hyper-responsive immunity and metastatic and nonmetastatic tumor rejection along with delays in outgrowth of spontaneous tumors. Such tumor resistance is mediated by activated CD8+ T cells and NK cells. Thus, Cbl-b is a promising target for cancer immunotherapy agents.

### SUMMARY

Provided herein are compounds that function to recruit Cbl-b protein or a mutated version thereof to an E3 ubiquitin ligase for targeted ubiquitination and subsequent proteasomal degradation. Such compounds include those having the chemical structure I or II:

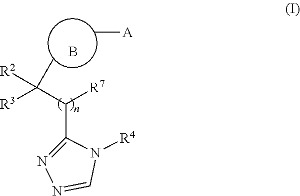

and pharmaceutically acceptable salt and compositions thereof, wherein A, R², R³, R⁴, R⁷, and n are as defined herein. Methods for preparing the compounds of the chemical structure I or II and methods for treating conditions responsive to the modulation of Cbl-b using the disclosed compounds, pharmaceutically acceptable salts, and compositions thereof are also included.

### DETAILED DESCRIPTION

### 1. General Description of Compounds

In a first embodiment, provided herein is compound having the chemical structure I:

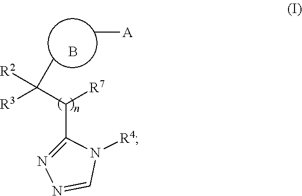

or a pharmaceutically acceptable salt thereof, wherein:
A is

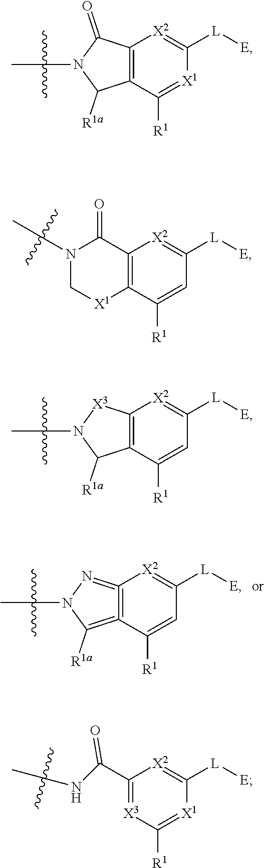

Ring B is phenyl or a 5 to 6-membered monocyclic heteroaryl;
X¹ X², and X³ are each independently CH or N;
X³ is 0 or 8;
each R^{1a} is independently hydrogen or optionally substituted C₁-C₄ alkyl;
R¹ is selected from hydrogen, optionally substituted C₁-C₄ alkyl and optionally substituted (C₃-C₅)cycloalkyl;
R² and R³ are each independently selected from hydrogen, optionally substituted C₁-C₄ alkyl, optionally substituted cycloalkyl, and optionally substituted heterocyclyl; or R² and R³ are taken together to form optionally substituted (C₃-C₄)cycloalkyl or optionally substituted 4-6 membered heterocyclyl;
R⁷ is hydrogen when n is 1, 2, or 3; or R² is hydrogen and R⁷ and R³, together with the atoms to which they are attached, form optionally substituted (C₃-C₄)cycloalkyl when n is 1;
R⁴ is selected from hydrogen, optionally substituted C₁-C₄ alkyl and optionally substituted (C₃-C₆)cycloalkyl;
n is 0, 1, 2, or 3;
L is a chemical linking moiety; and
E is a cereblon E3 ligase-binding moiety represented by the chemical structure:

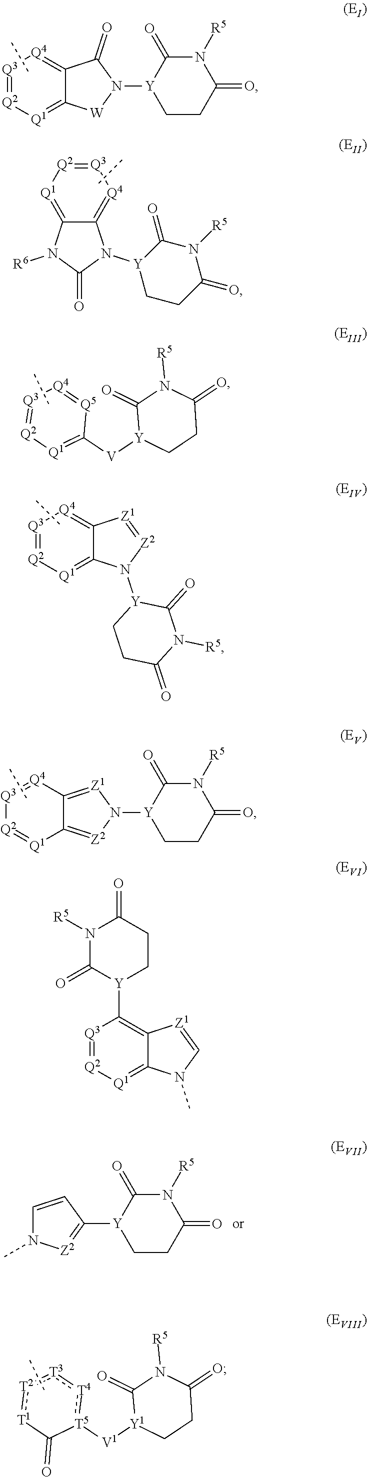

wherein:
W is CH₂, CHR^{v}, SO₂, or C(O);
Y and Y¹ are each independently N, CH, or CR^{y};
Q¹, Q², Q³, Q⁴, Q⁵, T¹, T², T³, T⁴, and T⁵ are each independently CH, CR^{w}, N, or NR^{w};
Z¹ and Z² are each independently CH, CR^{x} or N;
V is absent or is NRⁿ or C(O)NR^{z};
V¹ is absent or C₁₋₄ alkylene;
R⁵ and R⁶ are each independently hydrogen or optionally substituted C₁₋₄ alkyl;
R^{v}, R^{y}, and R^{x} are each independently selected from halo, optionally substituted C₁₋₄ alkyl, optionally substituted C₁₋₄ alkoxy, cyano, OH, —NH (optionally substituted C₁₋₄ alkyl), and —NH (optionally substituted C₁₋₄ alkyl)₂;
R^{w} is absent, or selected from halo, optionally substituted C₁₋₄ alkyl, optionally substituted C₁₋₄ alkoxy, cyano, OH, —NH (optionally substituted C₁₋₄ alkyl), and —NH (optionally substituted C₁₋₄ alkyl)₂;
Rⁿ and R^{z} are each independently hydrogen or optionally substituted C₁₋₆ alkyl, optionally substituted cycloalkyl, or optionally substituted heterocyclyl; and
the dashed line indicates the part of the structure to which the chemical linking moiety (L) is attached.

Alternatively, as part of the first embodiment, provided herein is compound having the chemical structure I:

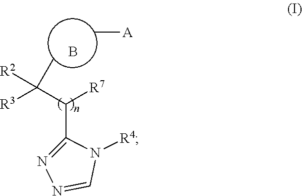

or a pharmaceutically acceptable salt thereof, wherein:
A is

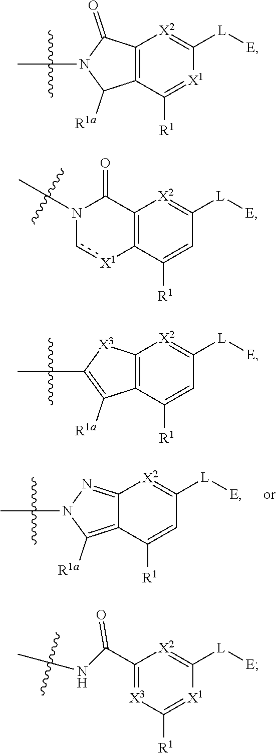

Ring B is phenyl or a 5 to 6-membered monocyclic heteroaryl;
X¹, X², and X³ are each independently CH or N;
X³ is O or S;
each R^{1a} is independently hydrogen or optionally substituted C₁-C₄ alkyl;
R¹ is selected from hydrogen, optionally substituted C₁-C₄ alkyl and optionally substituted (C₃-C₅)cycloalkyl;
R² and R³ are each independently selected from hydrogen, optionally substituted C₁-C₄ alkyl, optionally substituted cycloalkyl, and optionally substituted heterocyclyl; or R² and R³ are taken together to form optionally substituted (C₃-C₄)cycloalkyl or optionally substituted 4-6 membered heterocyclyl;
R⁷ is hydrogen when n is 1, 2, or 3; or R² is hydrogen and R⁷ and R³, together with the atoms to which they are attached, form optionally substituted (C₃-C₄)cycloalkyl when n is 1;
R⁴ is selected from hydrogen, optionally substituted C₁-C₄ alkyl and optionally substituted (C₃-C₆)cycloalkyl;
n is 0, 1, 2, or 3;
L is a chemical linking moiety; and
E is a cereblon E3 ligase-binding moiety represented by the chemical structure:

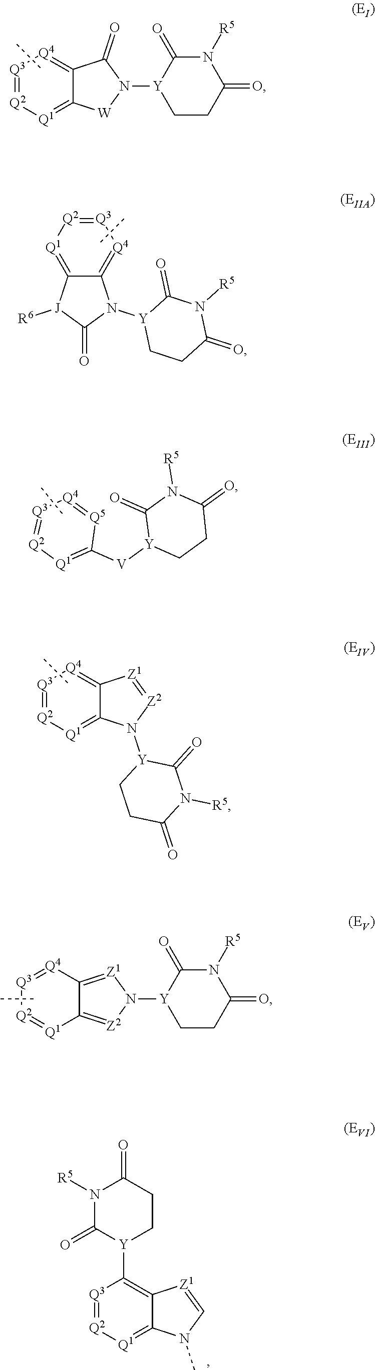

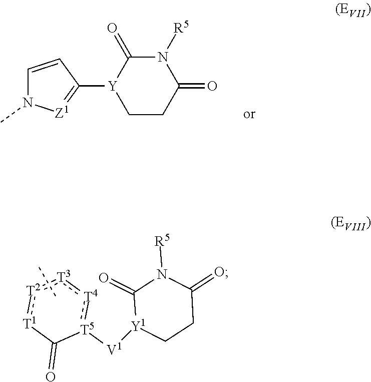

wherein:
W is CH₂, CHR^{v}, SO₂, or C(O);
Y and Y¹ are each independently N or CH;
J is CH or N;
Q¹, Q², Q³, Q⁴, Q⁵, T¹, T², T³, T⁴, and T⁵ are each independently CH, CR^{w}, N, or NR^{w};
Z¹ and Z² are each independently CH, CR^{x} or N;
V is absent or is NRⁿ or C(O)NR^{z};
V¹ is absent or C₁₋₄ alkylene;
R⁵ is H;
R⁶ is hydrogen or optionally substituted C₁₋₄ alkyl;
R^{v} and R^{x} are each independently selected from halo, optionally substituted C₁₋₄ alkyl, optionally substituted C₁₋₄ alkoxy, cyano, OH, —NH (optionally substituted C₁₋₄ alkyl), and —NH (optionally substituted C₁₋₄ alkyl)₂;
R^{w} is absent, or selected from halo, optionally substituted C₁₋₄ alkyl, optionally substituted C₁₋₄ alkoxy, cyano, OH, —NH (optionally substituted C₁₋₄ alkyl), and —NH (optionally substituted C₁₋₄ alkyl)₂;
Rⁿ and R^{z} are each independently hydrogen or optionally substituted C₁₋₆ alkyl, optionally substituted cycloalkyl, or optionally substituted heterocyclyl; and
the dashed line indicates the part of the structure to which the chemical linking moiety (L) is attached.

Alternatively, as part of the first embodiment, provided herein is compound having the chemical structure I:

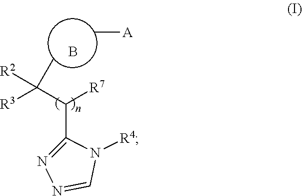

or a pharmaceutically acceptable salt thereof, wherein:
A is

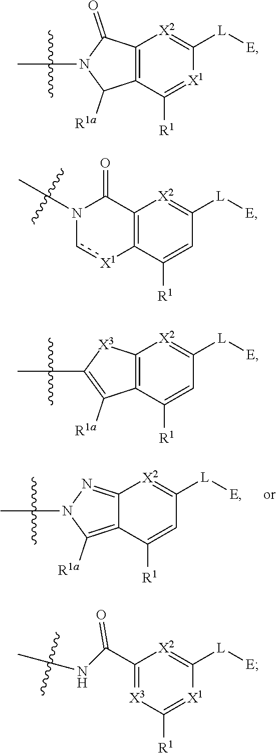

Ring B is phenyl or a 5 to 6-membered monocyclic heteroaryl;
X¹, X², and X³ are each independently CH or N;
X³ is O or S;
each R^{1a} is independently hydrogen or optionally substituted C₁-C₄ alkyl;
R¹ is selected from hydrogen, optionally substituted C₁-C₄ alkyl and optionally substituted (C₃-C₅)cycloalkyl;
R² and R³ are each independently selected from hydrogen, optionally substituted C₁-C₄ alkyl, optionally substituted cycloalkyl, and optionally substituted heterocyclyl; or R² and R³ are taken together to form optionally substituted (C₃-C₄)cycloalkyl or optionally substituted 4-6 membered heterocyclyl;
R⁷ is hydrogen when n is 1, 2, or 3; or R² is hydrogen and R⁷ and R³, together with the atoms to which they are attached, form optionally substituted (C₃-C₄)cycloalkyl when n is 1;
R⁴ is selected from hydrogen, optionally substituted C₁-C₄ alkyl and optionally substituted (C₃-C₆)cycloalkyl;
n is 0, 1, 2, or 3;
L is a chemical linking moiety; and
E is a cereblon E3 ligase-binding moiety represented by the chemical structure:

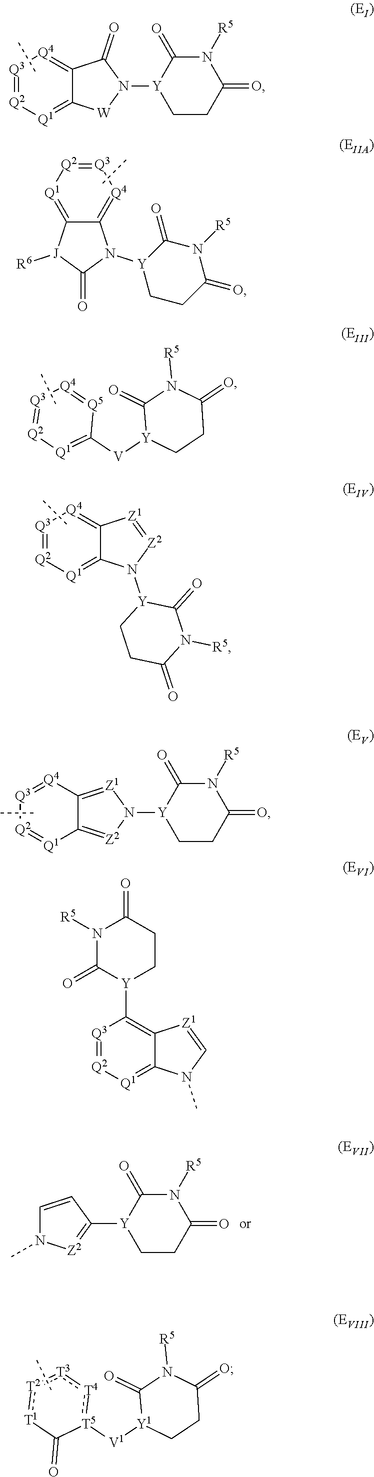

wherein:
W is CH₂, CHR^{v}, SO₂, or C(O);
Y and Y¹ are each independently N or CH;
J is CH or N;
Q¹, Q², Q³, Q⁴, Q⁵, T¹, T², T³, T⁴, and T⁵ are each independently CH, CR^{w}, N, or NR^{w};
Z¹ and Z² are each independently CH, CR^{x} or N;
V is absent or is NRⁿ or C(O)NR^{z};
V¹ is absent or C₁₋₄ alkylene;
R⁵ is H;
R⁶ is hydrogen or optionally substituted C₁₋₄ alkyl, C₁₋₄ haloalkyl, or C₃₋₄cycloalkyl;
R^{v} and R^{x} are each independently selected from halo, optionally substituted C₁₋₄ alkyl, optionally substituted C₁₋₄ alkoxy, cyano, OH, —NH (optionally substituted C₁₋₄ alkyl), and —NH (optionally substituted C₁₋₄ alkyl)₂;
R^{w} is absent, or selected from halo, optionally substituted C₁₋₄ alkyl, optionally substituted C₁₋₄ alkoxy, cyano, OH, —NH (optionally substituted C₁₋₄ alkyl), and —NH (optionally substituted C₁₋₄ alkyl)₂;
Rⁿ and R^{z} are each independently hydrogen or optionally substituted C₁₋₆ alkyl, optionally substituted cycloalkyl, or optionally substituted heterocyclyl; and
the dashed line indicates the part of the structure to which the chemical linking moiety (L) is attached.

### 2. Definitions

In instances where a term is not specifically defined herein, that term is given an art-recognized meaning by those of ordinary skill applying that term in context to its use in describing the present disclosure.

The articles “a” and “an” as used herein and in the appended claims are used herein to refer to one or to more than one (i.e., to at least one) of the grammatical object of the article unless the context clearly indicates otherwise. By way of example, “an element” means one element or more than one element, unless otherwise indicated.

All transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

When a range of carbon atoms is used herein, for example, C₁-C₆ or C₁₋₆, all ranges, as well as individual numbers of carbon atoms are encompassed. For example, “C₁₋₃” includes C₁₋₃, C₁₋₂, C₂₋₃, C₁, C₂, and C₃.

The terms “halo” and “halogen” refer to an atom selected from fluorine (fluoro, F), chlorine (chloro, Cl), bromine (bromo, Br), and iodine (iodo, I).

The term “alkyl” when used alone or as part of a larger moiety, such as “haloalkyl”, “hydroxyalkyl” and the like, means saturated straight-chain or branched monovalent hydrocarbon radical having, unless otherwise specified, from 1 to 20 carbon atoms such as C₁₋₁₀, C₁₋₆, or C₁₋₄. A C₁₋₆ alkyl includes e.g., methyl, ethyl, propyl (e.g., n-propyl, isopropyl), butyl (e.g., n-butyl, tert-butyl, sec-butyl, iso-butyl), pentyl (e.g., n-pentyl, 3-pentanyl, amyl, neopentyl, 3-methyl-2-butanyl, tertiary amyl), and hexyl (e.g., n-hexyl). It will be understood that when specified, optional substituents on an alkyl group may be present on any substitutable position.

The term “alkylene” refers to a bivalent alkyl group.

The term “haloalkyl” includes mono, poly, and perhaloalkyl groups where the halogens are independently selected from fluorine, chlorine, bromine, and iodine.

The term “hydroxyalkyl” includes mono, poly, and perhydroxy alkyl groups where one or more hydrogen atoms are replaced by OH.

The term “alkoxy” refers to an alkyl radical attached through an oxygen linking atom, represented by —Oalkyl. Non-limiting examples include methoxy, ethoxy, propoxy, 2-propoxy, butoxy, tert-butoxy, pentyloxy, and hexyloxy. It will be understood that when specified, optional substituents on an alkoxy group may be present on any substitutable position

The term “haloalkoxy” includes mono, poly, and perhaloalkoxy groups where the halogens are independently selected from fluorine, chlorine, bromine, and iodine.

The term “heteroaryl” refers to, unless otherwise specified, a 5-16 membered aromatic radical containing 1-4 heteroatoms selected from N, O, and S. In some instances, nitrogen atoms in a heteroaryl may be quarternized. Monocyclic heteroaryl includes, for example, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, etc. Bi-cyclic heteroaryls include groups in which a monocyclic heteroaryl ring is fused to one or more aryl or heteroaryl rings. Nonlimiting examples include indolyl, benzooxazolyl, benzooxodiazolyl, indazolyl, benzimidazolyl, benzthiazolyl, benzothiopheneyl, quinolinyl, quinazolinyl, quinoxalinyl, pyrrolopyridinyl, pyrrolopyrimidinyl, pyrrolopyridinyl, thienopyridinyl, thienopyrimidinyl, indolizinyl, purinyl, cinnolinyl, naphthyridinyl, and pteridinyl. It will be understood that when specified, optional substituents on a heteroaryl group may be present on any substitutable position and, include, e.g., the position at which the heteroaryl is attached (where valency permits).

The term “heterocyclyl” means, unless otherwise specified, a 4- to 12-membered saturated or partially unsaturated heterocyclic ring containing 1 to 4 heteroatoms independently selected from N, O, and S. A heterocyclyl ring can be attached to its pendant group at any heteroatom or carbon atom that results in a stable structure. A heterocyclyl group may be mono- or bicyclic (e.g., a bridged, fused, or spiro bicyclic ring). Examples of monocyclic saturated or partially unsaturated heterocyclic radicals include, without limitation, azetidinyl, tetrahydrofuranyl, tetrahydrothienyl, terahydropyranyl, pyrrolidinyl, pyrrolidonyl, piperidinyl, oxazolidinyl, piperazinyl, dioxanyl, dioxolanyl, morpholinyl, dihydrofuranyl, dihydropyranyl, dihydropyridinyl, tetrahydropyridinyl, dihydropyrimidinyl, tetrahydropyrimidinyl, dihydrooxadizolyl, and dihydroisoxazolyl. Bi-cyclic heterocyclyl groups include, e.g., unsaturated heterocyclic radicals fused to another unsaturated heterocyclic radical, cycloalkyl, aryl, or heteroaryl ring, such as for example, benzodioxolyl, dihydrobenzodioxinyl, dihydrobenzofuranyl, dihydrobenzothiophenyl, 5-oxa-2,6-diazaspiro[3.4]oct-6-enyl, 6-thia-2,7-diazaspiro[3.4]octanyl, 2,6-diazaspiro[3.3]heptanyl, spiro[indoline-3,3′-pyrrolidine]-yl, thiochromanyl, 7-azaspiro[3.5]nonanyl, 2,7-diazaspiro[3.5]nonanyl, 3-azaspiro[5.5]undecayl, 2-azaspiro[3.3]heptanyl, 3,9-diazaspiro[5.5]undecanyl, 6-azaspiro[3.4]octanyl and the like. It will be understood that when specified, optional substituents on a heterocyclyl group may be present on any substitutable position and, include, e.g., the position at which the heterocyclyl group is attached.

The term “spiro” refers to two rings that shares one ring atom (e.g., carbon).

The term “fused” refers to two rings that share two adjacent ring atoms with one another.

The term “bridged” refers to two rings that share three adjacent ring atoms with one another.

The term “cycloalkyl” refers to a saturated cyclic aliphatic monocyclic or bicyclic ring system, as described herein, having from, unless otherwise specified, 3 to 10 carbon ring atoms. Monocyclic cycloalkyl groups include, without limitation, cyclopropyl, cyclobutyl, cyclopentyl, cyclopentenyl, cyclohexyl, cyclohexenyl, cycloheptyl, cycloheptenyl, and cyclooctyl. It will be understood that when specified, optional substituents on a cycloalkyl or cycloaliphatic group may be present on any substitutable position and, include, e.g., the position at which the cycloalkyl group is attached.

The term “optionally substituted” means that one or more hydrogens of the designated moiety may be replaced with a suitable substituent. Unless otherwise indicated, an “optionally substituted” group may have a suitable substituent at each substitutable position of the group as valency permits. Optional substituents include, but are not limited to, one or more groups selected from cyano (—CN), halo, imino (═NH), nitro (—NO₂), oxo (═O), —C(O)Rⁱ, —C(O)ORⁱ, —C(O)NRⁱⁱRⁱⁱⁱ, —C(O)SRⁱ, —C(NRⁱ)NRⁱⁱRⁱⁱⁱ, —C(S)Rⁱ, —C(S)ORⁱ, —C(S)NRⁱⁱRⁱⁱⁱ, —ORⁱ, —OC(O)Rⁱ, —OC(O)ORⁱ, —OC(O)NRⁱⁱRⁱⁱⁱ, —OC(O)SRⁱ, —OC(NR)NRⁱⁱRⁱⁱⁱ, —OC(S)Rⁱ, —OC(S)OR, —OC(S)NRⁱⁱRⁱⁱⁱ, —OP(O)(ORⁱⁱ)ORⁱⁱⁱ, —OS(O)Rⁱ, —OS(O)₂Rⁱ, —OS(O)NRⁱⁱRⁱⁱⁱ, —OS(O)₂NRⁱⁱRⁱⁱⁱ, —NRⁱⁱRⁱⁱⁱ, —NRⁱC(O)R^{iv}, —NRⁱC(O)OR^{iv}, —NRⁱC(O)NRⁱⁱRⁱⁱⁱ, —NRⁱC(O)SR^{iv}, —NRⁱC(NR^{iv})NRⁱⁱRⁱⁱⁱ, —NRⁱC(S)R^{iv}, —NRⁱC(S)OR^{iv}, —NRⁱC(S)NRⁱⁱRⁱⁱⁱ, —NRⁱS(O)R^{iv}, —NRⁱS(O)₂R^{iv}, —NRⁱS(O)NRⁱⁱRⁱⁱⁱ, —NRⁱS(O)₂NRⁱⁱRⁱⁱⁱ, —SRⁱ, —S(O)Rⁱ, —S(O)₂Rⁱ, —S(O)NRⁱⁱRⁱⁱⁱ, —S(O)₂NRⁱⁱRⁱⁱⁱ, alkyl, heteroalkyl, alkenyl, alkynyl, cycloalkyl, aryl, heteroaryl, and heterocyclyl, wherein said alkyl, heteroalkyl, alkenyl, alkynyl, cycloalkyl, aryl, heteroaryl, and heterocyclyl are each further optionally substituted with one or more, in one embodiment, one, two, three, or four, substituents Q^{a}, wherein each Rⁱ, Rⁱⁱ, Rⁱⁱⁱ, and R^{iv} is independently alkyl, heteroalkyl, alkenyl, alkynyl, cycloalkyl, aryl, heteroaryl, or heterocyclyl, each of which is optionally substituted with one or more, in one embodiment, one, two, three, or four, substituents Q^{a} or Rⁱⁱ and Rⁱⁱⁱ together with the N atom to which they are attached form heterocyclyl optionally substituted with one or more, in one embodiment, one, two, three, or four, substituents Q^{a}, wherein each Q^{a} is independently selected from cyano, halo, imino, nitro, oxo, alkyl, heteroalkyl, alkenyl, alkynyl, cycloalkyl, C₆₋₁₄ aryl, heteroaryl, heterocyclyl, —C(O)R^{v}, —C(O)OR^{v}, —C(O)NR^{vi}R^{vii}, —C(O)SR^{v}, —C(NR^{v})NR^{vi}R^{vii}, —C(S)R^{v}, —C(S)OR^{v}, —C(S)NR^{vi}R^{vii}, —OR^{v}, —OC(O)R^{v}, —OC(O)OR^{v}, —OC(O)NR^{vi}R^{vii}, —OC(O)SR^{v}, —OC(NR^{v})NR^{vi}R^{vii}, —OC(S)R^{v}, —OC(S)OR^{v}, —OC(S)NR^{vi}R^{vii}, —OP(O)(OR^{v})OR^{vi}, —OS(O)R^{v}, —OS(O)₂R^{v}, —OS(O)NR^{vi}R^{vii}, —OS(O)₂NR^{v}R^{vii}, —NR^{vi}R^{vii}, —NR^{v}C(O)R^{vii}, —NR^{e}C(O)OR^{vi}, —NR^{v}C(O)NR^{vi}R^{vii}, —NR^{v}C(O)SR^{vi}, —NR^{v}C(NR^{vii})NR^{vi}R^{vii}, —NR^{v}C(S)R^{vii}, —NR^{v}C(S)OR^{vi}, —NR^{v}C(S)NR^{vi}R^{vii}, —NR^{c}S(O)R^{vii}, —NR^{c}S(O)₂R^{vii}, —NR^{c}S(O)NR^{vi}R^{vii}, —NR^{v}S(O)₂NR^{vi}R^{vii}, —SR^{v}, —S(O)R^{v}, —S(O)₂R^{v}, —S(O)NR^{vi}R^{vii}, and —S(O)₂NR^{vi}R^{vii}; wherein each R^{v}, R^{vi}, R^{vii}, and R^{vii} is independently alkyl, alkenyl, alkynyl, cycloalkyl, aryl, aralkyl, heteroaryl, or heterocyclyl; or (iii) R^{vi} and R^{vii} together with the N atom to which they are attached form heterocyclyl.

In certain aspects, where specified, one or more hydrogen atoms on a disclosed compound may be replaced with deuterium. Such deuterated compounds may have one or more improved pharmacokinetic or pharmacodynamic properties (e.g., longer half-life) compared to the equivalent “un-deuterated” compound.

One or more of the compounds described herein may exist in various tautomeric forms and are part of the present disclosure. The terms “tautomers” or “tautomeric” refer to two or more interconvertible compounds/substituents resulting from at least one formal migration of a hydrogen atom and at least one change in valency. All such isomeric forms of such compounds are expressly included. Thus, when a compound herein is represented by a structural formula or designated by a chemical name herein, all tautomeric forms which may exist for the compound are encompassed by the structural formula.

Compounds having one or more chiral centers can exist in various stereoisomeric forms. Stereoisomers are compounds that differ only in their spatial arrangement. Stereoisomers include all diastereomeric, enantiomeric, and epimeric forms as well as racemates and mixtures thereof. A “geometric isomer” refers to isomers that differ in the orientation of substituent group in relationship to a carbon-carbon double bond, a cycloalkyl ring, or a bridged bicyclic system. Atoms (other than H) on each side of a carbon-carbon double bond may be in an E (substituents are on opposite sides of the carbon-carbon double bond) or Z (substituents are oriented on the same side) configuration. “Cis” refers to substituents oriented on the same side of the ring, whereas “trans” refers to substituents oriented on opposite sides of the ring.

When the stereochemical configuration at a chiral center in a compound having one or more chiral centers is depicted by its chemical name (e.g., where the configuration is indicated in the chemical name by “R” or “S”) or structure (e.g., the configuration is indicated by “wedge” bonds), the enrichment of the indicated configuration relative to the opposite configuration is greater than 50%, 60%, 70%, 80%, 90%, 99% or 99.9%. “Enrichment of the indicated configuration relative to the opposite configuration” is a mole percent and is determined by dividing the number of compounds with the indicated stereochemical configuration at the chiral center(s) by the total number of all of the compounds with the same or opposite stereochemical configuration in a mixture.

When a geometric isomer is depicted by name or structure, the enrichment of the indicated isomer relative to the opposite isomer is greater than 50%, 60%, 70%, 80%, 90%, 99% or 99.9%. “Enrichment of the indicated isomer relative to the opposite isomer” is a mole percent and is determined by dividing the number of compounds with the indicated geometrical configuration by the total number of all of the compounds with the same or opposite geometrical configuration in a mixture.

When a disclosed compound is named or depicted by structure without indicating stereochemistry, it is understood that the name or the structure encompasses one of the possible stereoisomers or geometric isomers free of the others, or a mixture of the encompassed stereoisomers or geometric isomers.

The terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

The term “inhibit,” “inhibition” or “inhibiting” includes a decrease in the baseline activity of a biological activity or process.

As used herein, the terms “treatment,” “treat,” and “treating” refer to reversing, alleviating, delaying the onset of, or inhibiting the progress of a disease or disorder, or one or more symptoms thereof, as described herein. In some aspects, treatment may be administered after one or more symptoms have developed, i.e., therapeutic treatment. In other aspects, treatment may be administered in the absence of symptoms. For example, treatment may be administered to a susceptible individual prior to the onset of symptoms (e.g., in light of a history of symptoms and/or in light of exposure to a particular organism, or other susceptibility factors), i.e., prophylactic treatment. Treatment may also be continued after symptoms have resolved, for example to delay their recurrence.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Compounds of the present teachings with acidic groups such as carboxylic acids can form pharmaceutically acceptable salts with pharmaceutically acceptable base(s). Suitable pharmaceutically acceptable basic salts include e.g., ammonium salts, alkali metal salts (such as sodium and potassium salts) and alkaline earth metal salts (such as magnesium and calcium salts). Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, benzoates and salts with amino acids such as glutamic acid.

The terms “effective amount” and “therapeutically effective amount” of a compound described herein refers to an amount sufficient to induce a particular response in the subject, e.g., to provide a therapeutic benefit in the treatment of a condition described herein. The therapeutically effective amount of a compound of the disclosure or a pharmaceutically acceptable salt thereof may vary depending upon the intended application (in vitro or in vivo), the subject and disease condition being treated, e.g., the weight and age of the subject, the severity of the disease condition, the manner of administration and the like. Therapeutically effective amounts or doses of the compounds and pharmaceutically acceptable salts of the compounds described herein may be ascertained by routine methods such as modeling, dose escalation studies or clinical trials, and by taking into consideration factors such as, e.g., the mode or route of administration or drug delivery, the pharmacokinetics of the compound or salt, the severity and course of the disease or disorder, the subject's previous or ongoing therapy, the subject's health status and response to drugs, and the judgment of the treating physician. An illustrative example of a dose for a subject is in the range of from about 0.001 mg to about 1000 mg of compound (per day, in single or divided dosage units (e.g., BID, TID, QID).

The terms “condition”, “disease”, and “disorder” are used interchangeably herein and mean an abnormal condition that negatively affects the structure or function of all or part of a subject, and that is not immediately due to any external injury. In some embodiments, a disease is a medical condition, illness or sickness that is associated with one or more specific signs and symptoms.

The terms “administer,” “administering,” and “administration” refer to providing, implanting, absorbing, ingesting, injecting, inhaling, or otherwise introducing a compound described herein, or a pharmaceutically acceptable salt or pharmaceutical composition thereof, to, in or on a subject.

Unless otherwise indicated, the term “Cbl-b” includes both wild-type Cbl-b and mutant forms thereof.

A “Cbl-b related condition” refers to a condition that is responsive to the modulation of Cbl-b such as e.g., conditions which are modulated by degrading the Cbl-b protein. Cbl-b related conditions may arise from protein expression, overexpression, mutation, misfolding, or dysregulation (e.g., the amount of protein expressed in a patient is elevated).

### 3. Compounds and Compositions

In a second embodiment, the compound has the chemical structure II:

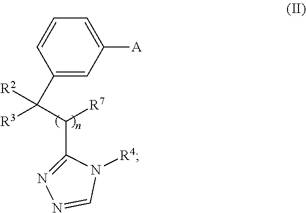

or a pharmaceutically acceptable salt thereof, and wherein the variables in chemical structure II are as described above for chemical structure I.

In a third embodiment, A in the compound having the chemical structure I or II, or a pharmaceutically acceptable salt thereof, is

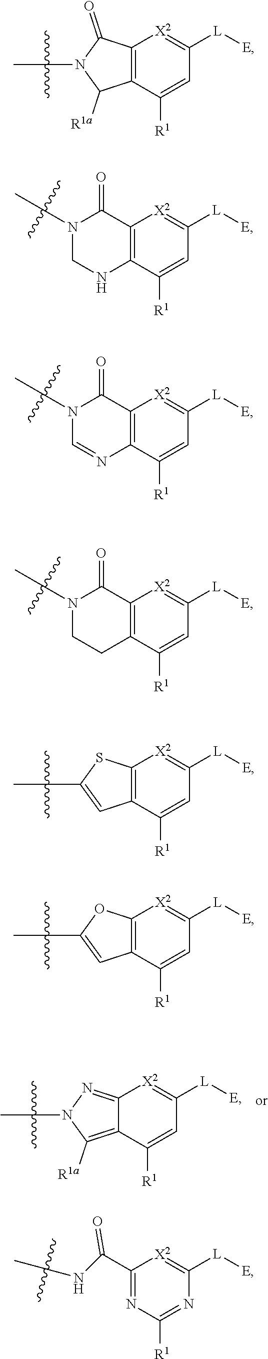

and wherein the remaining variables are as described above for chemical structure I or II.

In a fourth embodiment, A in the compound having the chemical structure I or II, or a pharmaceutically acceptable salt thereof, is

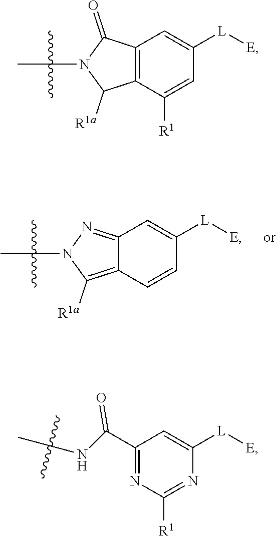

and wherein the remaining variables are as described above for chemical structure I or II.

In a fifth embodiment, n in the compound having the chemical structure I or II, or a pharmaceutically acceptable salt thereof, is 1, wherein the remaining variables are as described above for chemical structure I or II or the third or fourth embodiment. Alternatively, as part of the fifth embodiment, n in the compound having the chemical structure I or II, or a pharmaceutically acceptable salt thereof, is 0 or 1, wherein the remaining variables are as described above for chemical structure I or II or the third or fourth embodiment.

In a sixth embodiment, R^{1a} in the compound having the chemical structure I or II, or a pharmaceutically acceptable salt thereof, is hydrogen or C₁-C₄ alkyl, wherein the remaining variables are as described above for chemical structure I or II or any one of the third to fifth embodiments. Alternatively, as part of a sixth embodiment, R^{1a} in the compound having the chemical structure I or II, or a pharmaceutically acceptable salt thereof, is hydrogen, wherein the remaining variables are as described above for chemical structure I or II or any one of the third to fifth embodiments.

In a seventh embodiment, R¹ in the compound having the chemical structure I or II, or a pharmaceutically acceptable salt thereof, is selected from halo(C₁-C₄ alkyl) and (C₃-C₅)cycloalkyl, wherein the remaining variables are as described above for chemical structure I or II or any one of the third to sixth embodiments. Alternatively, as part of a seventh embodiment, R¹ in the compound having the chemical structure I or II, or a pharmaceutically acceptable salt thereof, is selected from CH₃, CF₃ and cyclopropyl, wherein the remaining variables are as described above for chemical structure I or II or any one of the third to sixth embodiments. In another alternative, as part of a seventh embodiment, R¹ in the compound having the chemical structure I or II, or a pharmaceutically acceptable salt thereof, is CF₃, wherein the remaining variables are as described above for chemical structure I or II or any one of the third to sixth embodiments.

In an eighth embodiment, R⁴ in the compound having the chemical structure I or II, or a pharmaceutically acceptable salt thereof, is selected from hydrogen, C₁-C₄ alkyl, halo(C₁-C₄ alkyl) and (C₃-C₆)cycloalkyl, wherein the remaining variables are as described above for chemical structure I or II or any one of the third to seventh embodiments. Alternatively, as part of an eighth embodiment, R⁴ in the compound having the chemical structure I or II, or a pharmaceutically acceptable salt thereof, is C₁-C₄ alkyl, wherein the remaining variables are as described above for chemical structure I or II or any one of the third to seventh embodiments. In another alternative, as part of an eighth embodiment, R⁴ in the compound having the chemical structure I or II, or a pharmaceutically acceptable salt thereof, is CH₃, wherein the remaining variables are as described above for chemical structure I or II or any one of the third to seventh embodiments.

In a ninth embodiment, R² in the compound having the chemical structure I or II, or a pharmaceutically acceptable salt thereof, is halo(C₁-C₄ alkyl), cyano(C₁-C₄ alkyl), hydroxy(C₁-C₄ alkyl), (C₃-C₄)cycloalkyl, or 4- to 6-membered heterocyclyl, wherein said (C₃-C₄)cycloalkyl and 4- to 6-membered heterocyclyl are each optionally substituted with 1 to 3 groups selected from C₁-C₄ alkyl, halo(C₁-C₄ alkyl), cyano(C₁-C₄ alkyl), cyano, halo, C₁-C₄ alkoxy, and halo(C₁-C₄ alkoxy), wherein the remaining variables are as described above for chemical structure I or II or any one of the third to eighth embodiments. Alternatively, as part of a ninth embodiment, R² in the compound having the chemical structure I or II, or a pharmaceutically acceptable salt thereof, is hydroxy(C₁-C₄ alkyl), C₁-C₄ alkyl, cyclobutyl, or tetrahydropyranyl, wherein the remaining variables are as described above for chemical structure I or II or any one of the third to eighth embodiments. In another alternative, as part of a ninth embodiment, R² in the compound having the chemical structure I or II, or a pharmaceutically acceptable salt thereof, is —CH₃—CH₂OH, cyclobutyl, or tetrahydropyranyl, wherein the remaining variables are as described above for chemical structure I or II or any one of the third to eighth embodiments.

In a tenth embodiment, R³ in the compound having the chemical structure I or II, or a pharmaceutically acceptable salt thereof, is hydrogen or C₁-C₄ alkyl, wherein the remaining variables are as described above for chemical structure I or II or any one of the third to ninth embodiments. Alternatively, as part of a tenth embodiment, R³ in the compound having the chemical structure I or II, or a pharmaceutically acceptable salt thereof, is hydrogen or CH₃, wherein the remaining variables are as described above for chemical structure I or II or any one of the third to ninth embodiments.

In an eleventh embodiment, R² and R³ in the compound having the chemical structure I or II, or a pharmaceutically acceptable salt thereof, are taken together to form (C₃-C₄)cycloalkyl or 4-membered heterocyclyl each optionally substituted with 1 to 3 groups selected from C₁-C₄ alkyl, halo(C₁-C₄ alkyl), cyano(C₁-C₄ alkyl), cyano, halo, C₁-C₄ alkoxy, and halo(C₁-C₄ alkoxy), wherein the remaining variables are as described above for chemical structure I or II or any one of the third to eighth embodiments. Alternatively, as part of an eleventh embodiment, R² and R³ in the compound having the chemical structure I or II, or a pharmaceutically acceptable salt thereof, are taken together to form cyclopropyl, cyclobutyl, or oxetanyl each optionally substituted with 1 to 3 groups selected from C₁-C₄ alkyl, halo(C₁-C₄ alkyl), cyano(C₁-C₄ alkyl), cyano, halo, C₁-C₄ alkoxy, and halo(C₁-C₄ alkoxy), wherein the remaining variables are as described above for chemical structure I or II or any one of the third to eighth embodiments. In another alternative, as part of an eleventh embodiment, R² and R³ in the compound having the chemical structure I or II, or a pharmaceutically acceptable salt thereof, are taken together to form cyclopropyl, cyclobutyl, or oxetanyl each optionally substituted with 1 to 3 groups selected from selected C₁-C₄ alkyl, halo(C₁-C₄ alkyl), cyano(C₁-C₄ alkyl) and cyano, wherein the remaining variables are as described above for chemical structure I or II or any one of the third to eighth embodiments. In yet another alternative, as part of an eleventh embodiment, R² and R³ in the compound having the chemical structure I or II, or a pharmaceutically acceptable salt thereof, are taken together to form cyclopropyl, cyclobutyl, or oxetanyl each optionally substituted with CH₃ or cyano, wherein the remaining variables are as described above for chemical structure I or II or any one of the third to eighth embodiments.

In a twelfth embodiment, E in the compound having the chemical structure I or II, or a pharmaceutically acceptable salt thereof, is represented by the chemical structure:

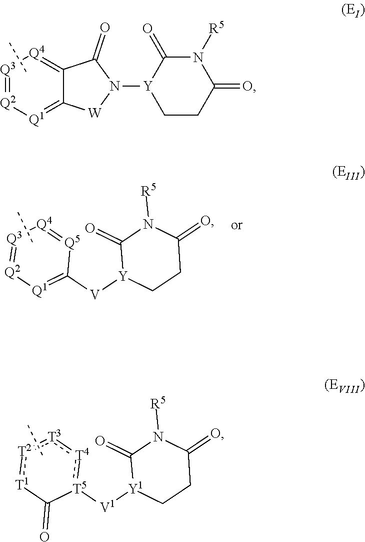

wherein the remaining variables are as described above for chemical structure I or II or any one of the third to eleventh embodiments. Alternatively, as part of a twelfth embodiment, E in the compound having the chemical structure I or II, or a pharmaceutically acceptable salt thereof, is represented by the chemical structure:

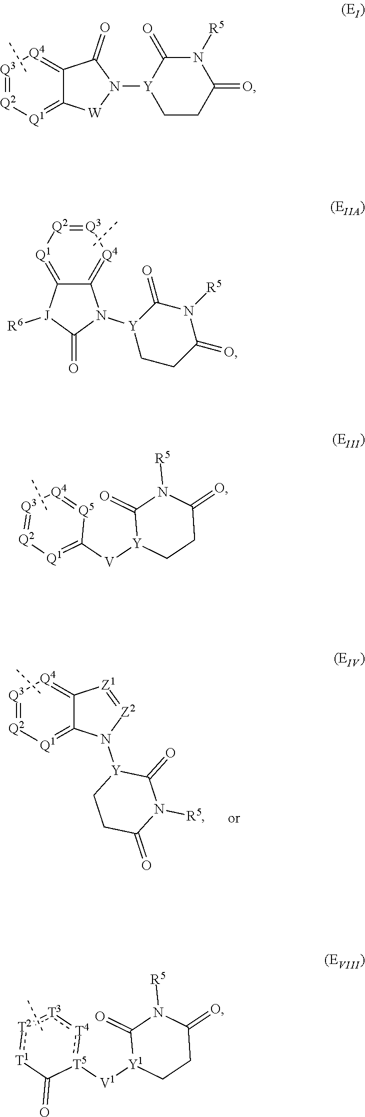

wherein the remaining variables are as described above for chemical structure I or II or any one of the third to eleventh embodiments. Alternatively, as part of a twelfth embodiment, E in the compound having the chemical structure I or II, or a pharmaceutically acceptable salt thereof, is represented by the chemical structure:

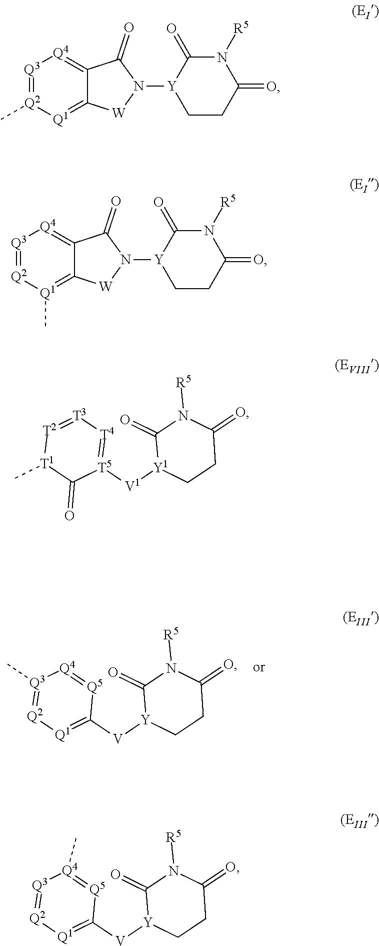

(E_{III}″), wherein the remaining variables are as described above for chemical structure I or II or any one of the third to eleventh embodiments. Alternatively, as part of a twelfth embodiment, E in the compound having the chemical structure I or II, or a pharmaceutically acceptable salt thereof, is represented by the chemical structure:

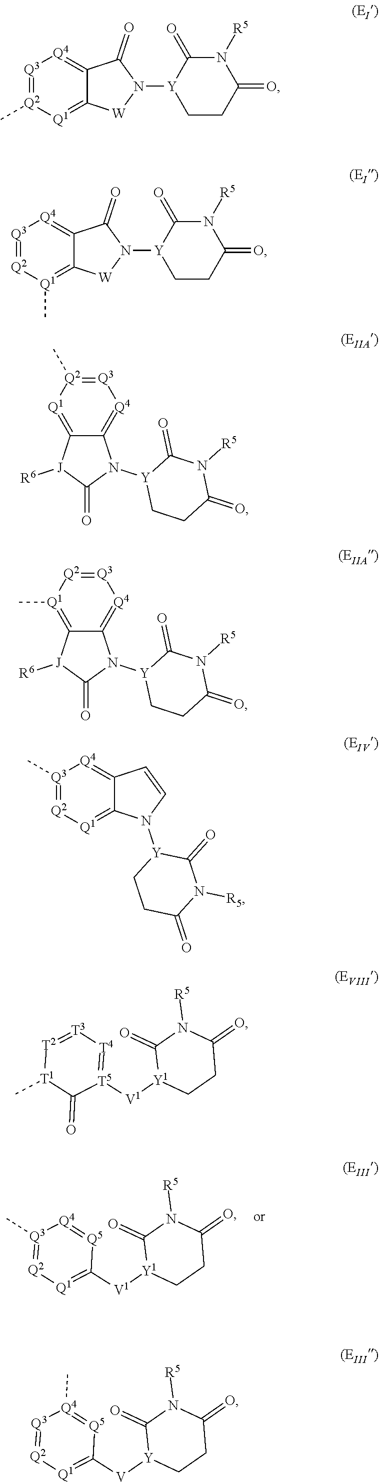

wherein the remaining variables are as described above for chemical structure I or II or any one of the third to eleventh embodiments. In another alternative, as part of the twelfth embodiment, E in the compound having the chemical structure I or II, or a pharmaceutically acceptable salt thereof, is represented by the chemical structure:

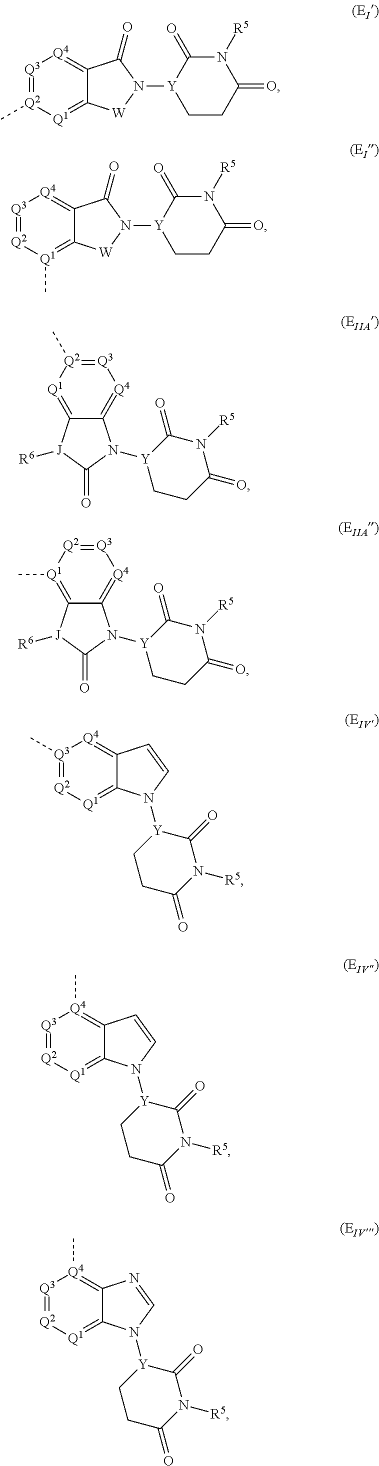

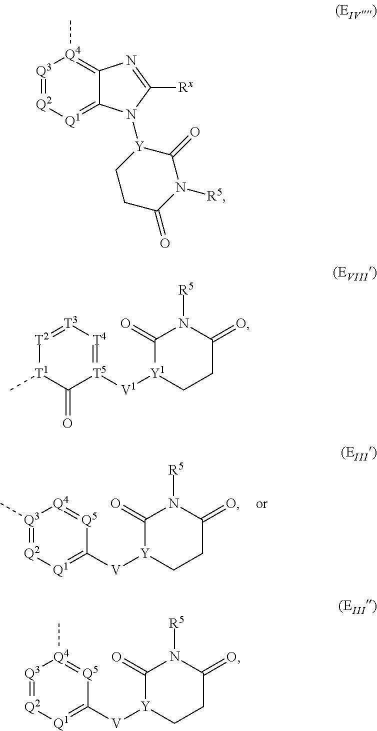

wherein the remaining variables are as described above for chemical structure I or II or any one of the third to eleventh embodiments.

In a thirteenth embodiment, Y in the compound having the chemical structure I or II, or a pharmaceutically acceptable salt thereof, is CH, wherein the remaining variables are as described above for chemical structure I or II or any one of the third to twelfth embodiments. Alternatively, in a thirteenth embodiment, Y in the compound having the chemical structure I or II, or a pharmaceutically acceptable salt thereof, is N, wherein the remaining variables are as described above for chemical structure I or II or any one of the third to twelfth embodiments. In an alternative thirteenth embodiment, J in the compound having the chemical structure I or II, or a pharmaceutically acceptable salt thereof, is N, wherein the remaining variables are as described above for chemical structure I or II or any one of the third to twelfth or alternative thirteenth embodiments.

In a fourteenth embodiment, R⁵ in the compound having the chemical structure I or II, or a pharmaceutically acceptable salt thereof, is hydrogen, wherein the remaining variables are as described above for chemical structure I or II or any one of the third to thirteenth embodiments. Alternatively, in a fourteenth embodiment, R⁶ in the compound having the chemical structure I or II, or a pharmaceutically acceptable salt thereof, is H, C₁₋₄alkyl, C₁₋₄haloalkyl, or C₃₋₄cycloakyl, wherein the remaining variables are as described above for chemical structure I or II or any one of the third to thirteenth or alternative fourteenth embodiments. In another alternative, as part of the fourteenth embodiment, R⁶ in the compound having the chemical structure I or II, or a pharmaceutically acceptable salt thereof, is H or C₁₋₄alkyl, wherein the remaining variables are as described above for chemical structure I or II or any one of the third to thirteenth or alternative fourteenth embodiments. fourteenth embodiment, R⁶ in the compound having the chemical structure I or II, or a pharmaceutically acceptable salt thereof, is H, —CH₃, —CH(CH₃)₂, —CHF₂, or cyclopropyl, wherein the remaining variables are as described above for chemical structure I or II or any one of the third to thirteenth or alternative fourteenth embodiments. fourteenth embodiment, R⁶ in the compound having the chemical structure I or II, or a pharmaceutically acceptable salt thereof, is H, —CH₃, or —CH(CH₃)₂, wherein the remaining variables are as described above for chemical structure I or II or any one of the third to thirteenth or alternative fourteenth embodiments.

In a fifteenth embodiment, W in the compound having the chemical structure I or II, or a pharmaceutically acceptable salt thereof, is CH₂, —CH(C₁₋₄ alkyl), or C(O), wherein the remaining variables are as described above for chemical structure I or II or any one of the third to fourteenth embodiments.

In a sixteenth embodiment, V in the compound having the chemical structure I or II, or a pharmaceutically acceptable salt thereof, is absent or C(O)NH, wherein the remaining variables are as described above for chemical structure I or II or any one of the third to fifteenth embodiments.

In a seventeenth embodiment, V¹ in the compound having the chemical structure I or II, or a pharmaceutically acceptable salt thereof, is CH₂, wherein the remaining variables are as described above for chemical structure I or II or any one of the third to sixteenth embodiments.

In an eighteenth embodiment, Y¹ in the compound having the chemical structure I or II, or a pharmaceutically acceptable salt thereof, is CH, wherein the remaining variables are as described above for chemical structure I or II or any one of the third to seventeenth embodiments. Alternatively, in an eighteenth embodiment, Yⁱ in the compound having the chemical structure I or II, or a pharmaceutically acceptable salt thereof, is N, wherein the remaining variables are as described above for chemical structure I or II or any one of the third to seventeenth embodiments.

In a nineteenth embodiment, T¹ in the compound having the chemical structure I or II, or a pharmaceutically acceptable salt thereof, is N, wherein the remaining variables are as described above for chemical structure I or II or any one of the third to eighteenth embodiments.

In a twentieth embodiment, T², T³, and T⁴ in the compound having the chemical structure I or II, or a pharmaceutically acceptable salt thereof, are each CH, wherein the remaining variables are as described above for chemical structure I or II or any one of the third to nineteenth embodiments.

In a twenty-first embodiment, Q¹ for the chemical structure E_{I}, E_{I}′, or E_{I}″, is N, CR^{w}, or CH, wherein the remaining variables are as described above for chemical structure I or II or any one of the third to twentieth embodiments.

In a twenty-second embodiment, Q², Q³, and Q⁴ for the chemical structure E_{I}, E_{I}′, or E_{I}″ are each CH or CR^{w}, wherein the remaining variables are as described above for chemical structure I or II or any one of the third to twenty-first embodiments. Alternatively, as part of the twenty-second embodiment, Q¹, Q², Q³, and Q⁴ for the chemical structure E_{I}, E_{I}′, E_{I}″, E_{IIA}′, or E_{IV}′ are each CH or CR^{w}, wherein the remaining variables are as described above for chemical structure I or II or any one of the third to twenty-first embodiments.

In a twenty-third embodiment, Q⁵ for the chemical structure E_{III}, E_{III}′, or E_{III}″ is CH or CR^{w}, wherein the remaining variables are as described above for chemical structure I or II or any one of the third to twenty-second embodiments.

In a twenty-fourth embodiment, Q², Q³, Q⁴, and Q⁵ for the chemical structure E_{III}, E_{III}′, or E_{III}″ are each CH or C, wherein the remaining variables are as described above for chemical structure I or II or any one of the third to twenty-third embodiments. Alternatively, as part of the twenty-fourth embodiment, Q¹, Q², Q³, Q⁴, and Q⁵ for the chemical structure E_{III}, E_{III}′, or E_{III}″ are each CH, N, or C, wherein the remaining variables are as described above for chemical structure I or II or any one of the third to twenty-third embodiments.

In a twenty-fifth embodiment, R^{w} in the compound having the chemical structure I or II, or a pharmaceutically acceptable salt thereof, is absent or is halo, C₁-C₄ alkoxy, or C₁-C₄ alkyl, wherein the remaining variables are as described above for chemical structure I or II or any one of the third to twenty-fourth embodiments. Alternatively, as part of a twenty-third embodiment, R^{w} in the compound having the chemical structure I or II, or a pharmaceutically acceptable salt thereof, is absent or is fluoro, chloro, OCH₃, or CH₃, wherein the remaining variables are as described above for chemical structure I or II or any one of the third to twenty-fourth embodiments. Alternatively, as part of a twenty-third embodiment, R^{x} in the compound having the chemical structure I or II, or a pharmaceutically acceptable salt thereof, is C₁₋₄alkoxy, wherein the remaining variables are as described above for chemical structure I or II or any one of the third to twenty-fourth or alternative twenty-fifth embodiments. Alternatively, as part of a twenty-third embodiment, R^{x} in the compound having the chemical structure I or II, or a pharmaceutically acceptable salt thereof, is —OCH₃, wherein the remaining variables are as described above for chemical structure I or II or any one of the third to twenty-fourth or alternative twenty-fifth embodiments.

In a twenty-sixth embodiment, E in the compound having the chemical structure I or II, or a pharmaceutically acceptable salt thereof, is selected from:

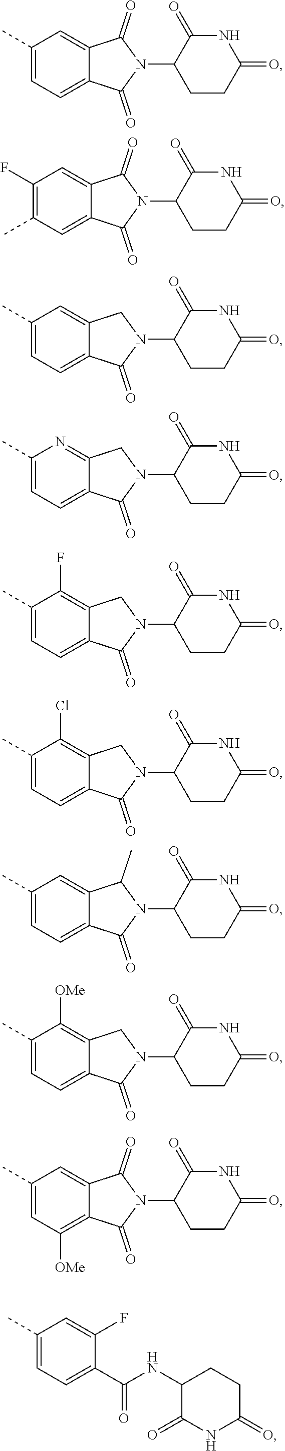

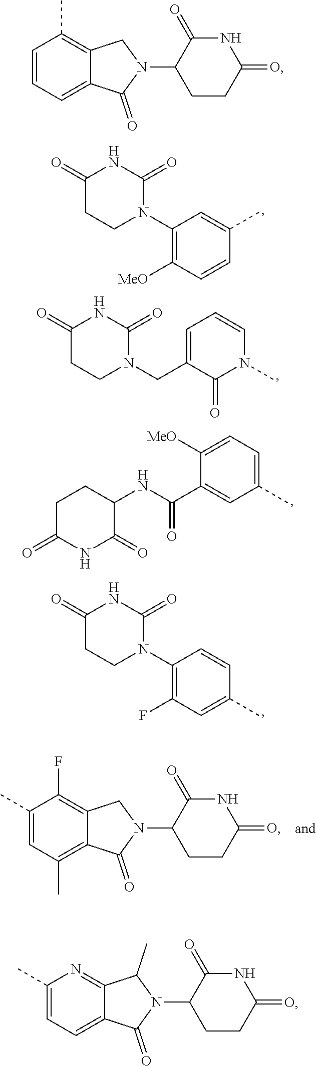

wherein the remaining variables are as described above for chemical structure I or II or any one of the third to twenty-fifth embodiments. Alternatively, as part of the twenty-sixth embodiment, E in the compound having the chemical structure I or II, or a pharmaceutically acceptable salt thereof, is selected from:

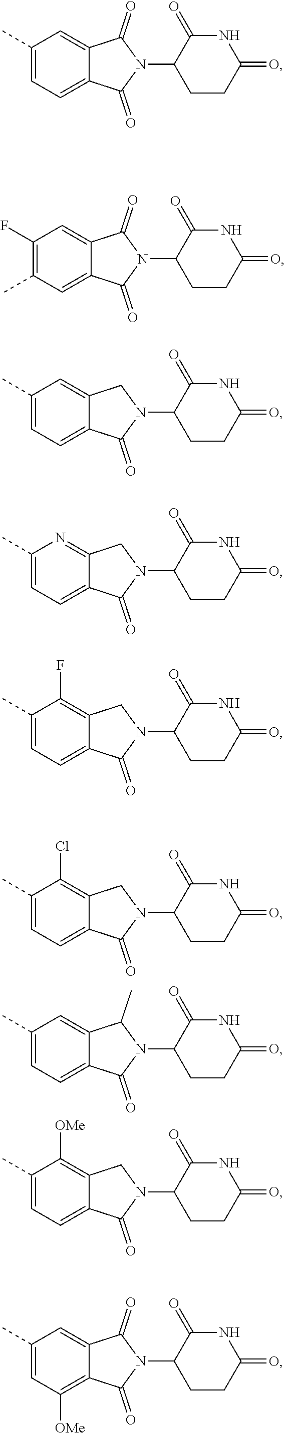

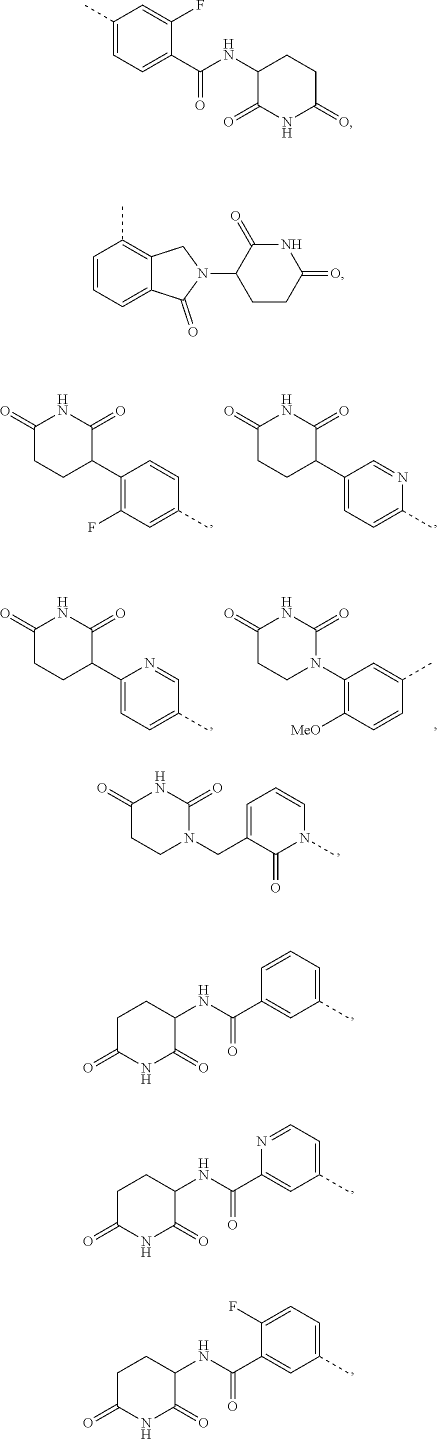

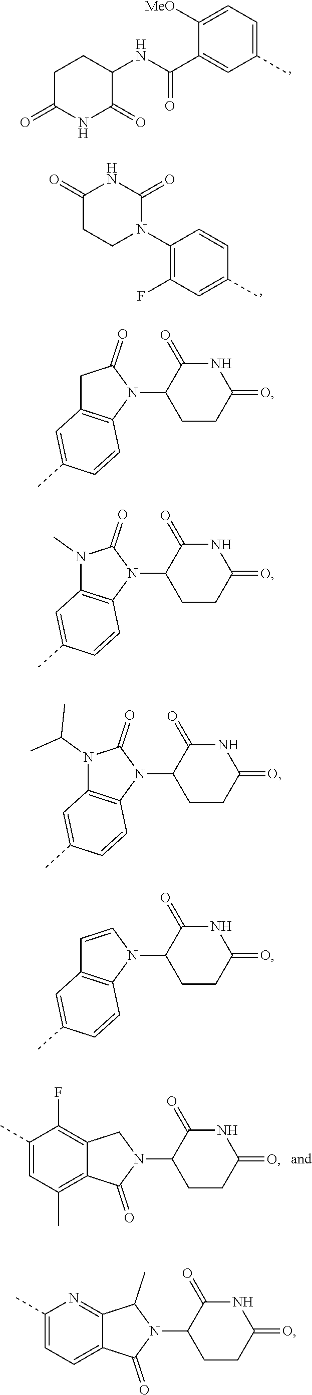

wherein the remaining variables are as described above for chemical structure I or II or any one of the third to twenty-fifth embodiments. In another alternative, as part of the twenty-sixth embodiment, E in the compound having the chemical structure I or II, or a pharmaceutically acceptable salt thereof, is selected from:

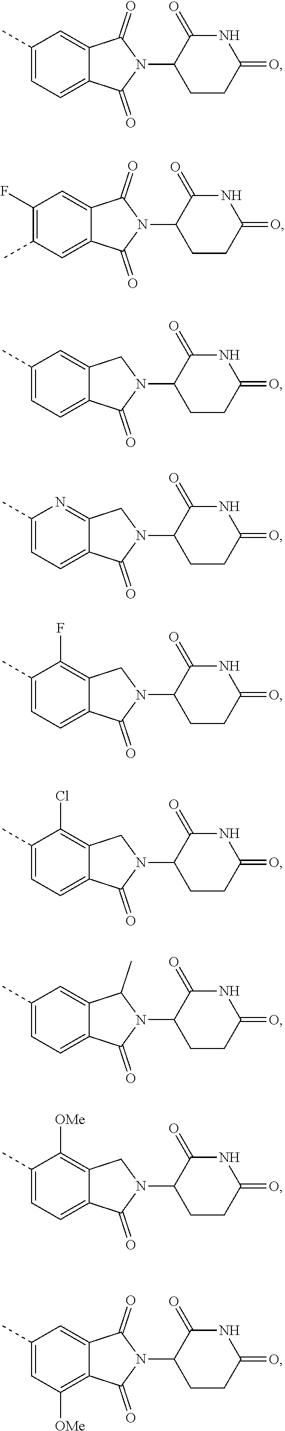

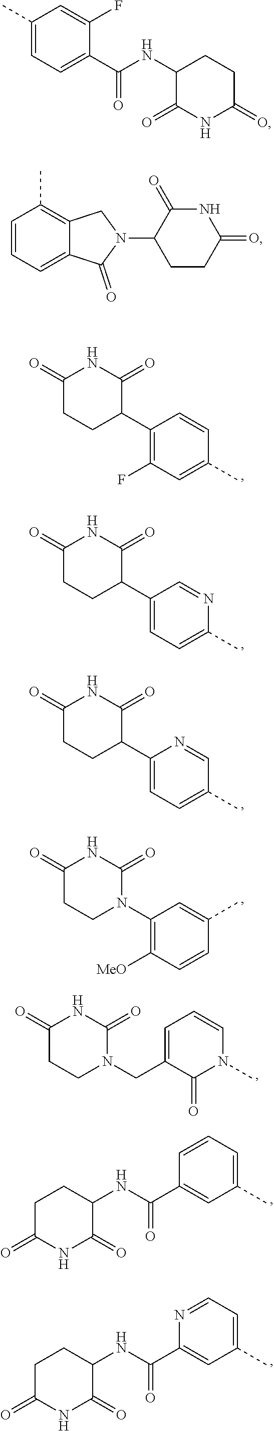

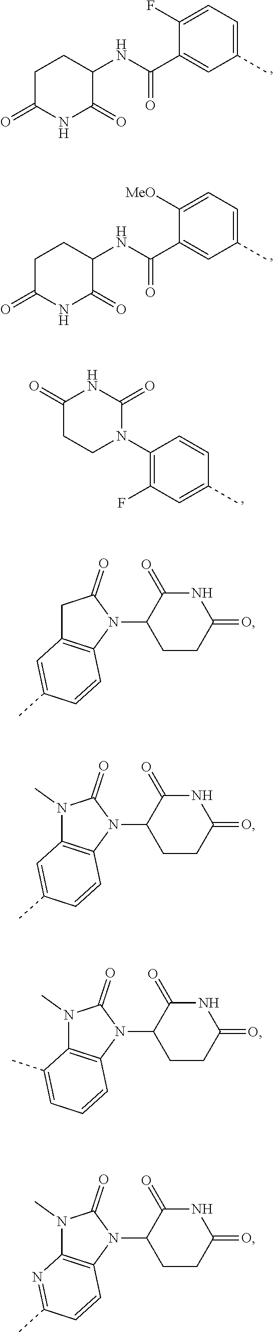

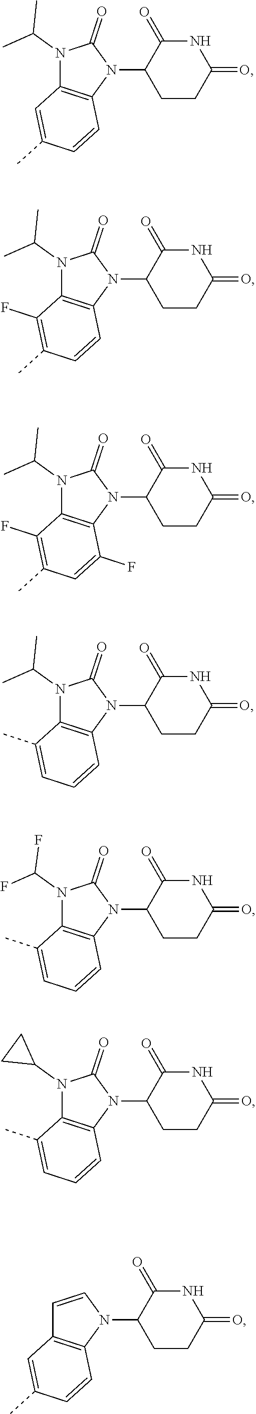

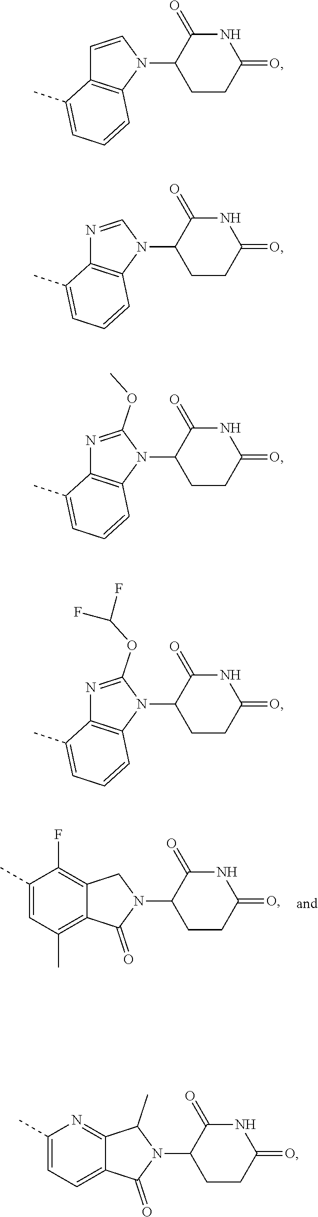

wherein the remaining variables are as described above for chemical structure I or II or any one of the third to twenty-fifth embodiments.

In a twenty-seventh embodiment, chemical linking moiety (L) in the compound having the chemical structure I or II, or a pharmaceutically acceptable salt thereof, is represented by the chemical structure:

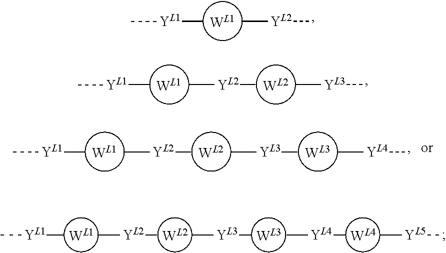

wherein:
Y^{L1}, Y^{L2}, Y^{L3}, Y^{L4} and Y^{L5} are each independently absent or selected from O, NR^{Y}, S, SO, SO₂, SO₂NR^{Y}, C(O), C(O)O, C(O)NR^{Y}, and an optionally substituted C₁₋₆ alkylene, wherein said C₁₋₆ alkylene may also be optionally interrupted by one or more O, NH, and N(C₁₋₄ alkyl), and wherein two hydrogens on the same carbon of said C₁₋₆ alkylene may be taken together to form oxo;
R^{Y} is H or C₁₋₄ alkyl; and
W^{L1}, W^{L2}, W^{L3}, and W^{L4} are each independently selected from phenyl, heterocyclyl, heteroaryl, and cycloalkyl, each of which are optionally substituted and wherein the remaining variables are as described above for chemical structure I or II or any one of the third to twenty-sixth embodiments.

In a twenty-eighth embodiment, Y^{L1}, Y^{L2}, Y^{L3}, Y^{L4}, and Y^{L5} in the twenty-seventh embodiment, are each independently absent or selected from O, NH, N(C₁₋₄ alkyl), and a C₁₋₆ alkylene optionally interrupted by one or more O, NH, and N(C₁₋₄ alkyl), and wherein two hydrogens on the same carbon of said C₁₋₆ alkylene may be taken together to form oxo; and W^{L1}, W^{L2}, W^{L3}, and W^{L4} are each independently selected from heterocyclyl, heteroaryl, and cycloalkyl, each of which are optionally substituted with 1 or 2 C₁₋₄ alkyl, wherein the remaining variables are as described above for chemical structure I or II or any one of the third to twenty-sixth embodiments. Alternatively, as part of the twenty-eighth embodiment, Y^{L1}, Y^{L2}, Y^{L3}, Y^{L4} and Y^{L5} in the twenty-seventh embodiment, are each independently absent or selected from O, NH, N(C₁₋₄ alkyl), and a C₁₋₆ alkylene optionally interrupted by one or more O, NH, and N(C₁₋₄ alkyl), and wherein two hydrogens on the same carbon of said C₁₋₆ alkylene may be taken together to form oxo; and W^{L1}, W^{L2}, W^{L3}, and W^{L4} are each independently selected from heterocyclyl, heteroaryl, and cycloalkyl, each of which are optionally substituted with 1 or 2 C₁₋₄ alkyl, or two substituents together form C₃₋₄cycloalkyl, wherein the remaining variables are as described above for chemical structure I or II or any one of the third to twenty-sixth embodiments. In another alternative, as part of the twenty-eighth embodiment, Y^{L1}, Y^{L2}, Y^{L3}, Y^{L4}, and Y^{L5} in the twenty-seventh embodiment, are each independently absent or selected from O, NH, N(C₁₋₄ alkyl), and a C₁₋₆ alkylene optionally interrupted by one or more O, NH, and N(C₁₋₄ alkyl), and wherein two hydrogens on the same carbon of said C₁₋₆ alkylene may be taken together to form oxo; and W^{L1}, W^{L2}, W^{L3}, and W^{L4} are each independently selected from phenyl, heterocyclyl, heteroaryl, and cycloalkyl, each of which are optionally substituted with 1 or 2 C₁₋₄ alkyl, or two substituents together form C₃₋₄cycloalkyl or 4- to 6-membered heterocyclyl, wherein the remaining variables are as described above for chemical structure I or II or any one of the third to twenty-sixth embodiments.

In a twenty-ninth embodiment, Y^{L1} in the twenty-seventh embodiment is absent or C₁₋₆ alkylene optionally interrupted by one or more O, NH, and N(C₁₋₄ alkyl), wherein the remaining variables are as described above for chemical structure I or II or any one of the third to twenty-eighth embodiments.

In a thirtieth embodiment, W^{L1} in the twenty-seventh embodiment is selected from 4- to 11-membered heterocyclyl and 3- to 6-membered cycloalkyl, each of which is optionally substituted with 1 or 2 C₁₋₄ alkyl, wherein the remaining variables are as described above for chemical structure I or II or any one of the third to twenty-ninth embodiments. Alternatively, as part of a thirtieth embodiment, W^{L1} in the twenty-seventh embodiment is selected from 4- to 11-membered heterocyclyl and 3- to 6-membered cycloalkyl, each of which is optionally substituted with 1 or 2 C₁₋₄ alkyl, or two substituents together form C₃₋₄cycloalkyl, wherein the remaining variables are as described above for chemical structure I or II or any one of the third to twenty-ninth embodiments. In another alternative, as part of the thirtieth embodiment, W^{L1} in the twenty-seventh embodiment is selected from phenyl, 4- to 11-membered heterocyclyl, and 3-to 6-membered cycloalkyl, each of which is optionally substituted with 1 or 2 C₁₋₄ alkyl, or two substituents together form C₃₋₄cycloalkyl or oxetanyl, wherein the remaining variables are as described above for chemical structure I or II or any one of the third to twenty-ninth embodiments. Alternatively, as part of a thirtieth embodiment, W^{L1} in the twenty-seventh embodiment is selected from cyclobutyl, cyclohexyl, azetidinyl, pyrrolidinyl, piperidinyl, piperazinyl, 3,9-diazaspiro[5.5]undecanyl, 2,7-diazaspiro[3.5]nonanyl, 2,6-diazaspiro[3.3]heptanyl, 2,6-diazaspiro[3.4]octanyl, and 2-azaspiro[3.3]heptanyl each of which are optionally substituted with 1 or 2 C₁₋₄ alkyl, wherein the remaining variables are as described above for chemical structure I or II or any one of the third to twenty-ninth embodiments. Alternatively, as part of a thirtieth embodiment, W^{L1} in the twenty-seventh embodiment is selected from cyclobutyl, cyclohexyl, azetidinyl, pyrrolidinyl, piperidinyl, piperazinyl, 1,4-diazepanyl, 2,5-diazabicyclo[2.2.1]heptanyl, 2,6-diazaspiro[3.4]octanyl, 2,7-diazaspiro[4.4]nonanyl, 3,9-diazaspiro[5.5]undecanyl, 2,7-diazaspiro[3.5]nonanyl, 2,6-diazaspiro[3.3]heptanyl, 2,6-diazaspiro[3.4]octanyl, and 2-azaspiro[3.3]heptanyl each of which are optionally substituted with 1 or 2 C₁₋₄ alkyl, or two substituents together form C₃₋₄cycloalkyl, wherein the remaining variables are as described above for chemical structure I or II or any one of the third to twenty-ninth embodiments. In another alternative, as part of a thirtieth embodiment, W^{L1} in the twenty-seventh embodiment is selected from cyclobutyl, cyclohexyl, azetidinyl, pyrrolidinyl, piperidinyl, piperazinyl, 1,4-diazepanyl, 2,5-diazabicyclo[2.2.1]heptanyl, 2,6-diazaspiro[3.4]octanyl, 2,7-diazaspiro[4.4]nonanyl, 3,9-diazaspiro[5.5]undecanyl, 2,7-diazaspiro[3.5]nonanyl, 2,6-diazaspiro[3.3]heptanyl, 2,6-diazaspiro[3.4]octanyl, and 2-azaspiro[3.3]heptanyl each of which are optionally substituted with 1 or 2 C₁₋₄ alkyl, or two substituents together form C₃₋₄cycloalkyl or oxetanyl, wherein the remaining variables are as described above for chemical structure I or II or any one of the third to twenty-ninth embodiments

In a thirty-first embodiment, Y^{L2} in the twenty-seventh embodiment is absent or selected from O and C₁₋₆ alkylene optionally interrupted by one or more O, NH, and N(C₁₋₄ alkyl), and wherein two hydrogens on the same carbon of said C₁₋₆ alkylene may be taken together to form oxo, wherein the remaining variables are as described above for chemical structure I or II or any one of the third to thirtieth embodiments.

In a thirty-second embodiment, W^{L2} in the twenty-seventh embodiment is selected from 5- to 6-membered heteroaryl, 4- to 11-membered heterocyclyl and 3- to 6-membered cycloalkyl, each of which is optionally substituted with 1 or 2 C₁₋₄ alkyl, wherein the remaining variables are as described above for chemical structure I or II or any one of the third to thirty-first embodiments. Alternatively, as part of a thirty-second embodiment, W^{L2} in the twenty-seventh embodiment is selected from cyclobutyl, cyclohexyl, azetidinyl, piperidinyl, piperazinyl, 3,9-diazaspiro[5.5]undecanyl, 2,7-diazaspiro[3.5]nonanyl, 2,6-diazaspiro[3.3]heptanyl, 3-azaspiro[5.5]undecayl, 2-azaspiro[3.3]heptanyl, and pyrimidinyl, each of which are optionally substituted with 1 or 2 C₁₋₄ alkyl, wherein the remaining variables are as described above for chemical structure I or II or any one of the third to thirty-first embodiments.

In a thirty-third embodiment, Y^{L3} in the twenty-seventh embodiment is absent or selected from O and a C₁₋₆ alkylene, wherein said C₁₋₆ alkylene may be optionally interrupted by O, NH, and N(C₁₋₄ alkyl), wherein the remaining variables are as described above for chemical structure I or II or any one of the third to thirty-second embodiments.

In a thirty-fourth embodiment, W^{L3} in the twenty-seventh embodiment is selected from 4- to 11-membered heterocyclyl and 3- to 6-membered cycloalkyl, each of which is optionally substituted with 1 or 2 C₁₋₄ alkyl, wherein the remaining variables are as described above for chemical structure I or II or any one of the third to thirty-third embodiments. Alternatively, as part of a thirty-fourth embodiment, W^{L} in the twenty-seventh embodiment is selected from cyclohexyl, azetidinyl, piperidinyl, piperazinyl, and 2,7-diazaspiro[3.5]nonanyl, each of which are optionally substituted with 1 or 2 C₁₋₄ alkyl, wherein the remaining variables are as described above for chemical structure I or II or any one of the third to thirty-third embodiments.

In a thirty-fifth embodiment, Y^{L4} in the twenty-seventh embodiment is absent or selected from O and a C₁₋₆ alkylene, wherein the remaining variables are as described above for chemical structure I or II or any one of the third to thirty-fourth embodiments.

In a thirty-sixth embodiment, W⁴ in the twenty-seventh embodiment is 4- to 7-membered heterocyclyl optionally substituted with 1 or 2 C₁₋₄ alkyl, wherein the remaining variables are as described above for chemical structure I or II or any one of the third to thirty-fifth embodiments. Alternatively, as part of a thirty-sixth embodiment, W¹⁴ in the twenty-seventh embodiment is selected from piperidinyl and piperazinyl, each of which are optionally substituted with 1 or 2 C₁₋₄ alkyl, wherein the remaining variables are as described above for chemical structure I or II or any one of the third to thirty-fifth embodiments.

In a thirty-seventh embodiment, YU in the twenty-seventh embodiment is absent, wherein the remaining variables are as described above for chemical structure I or II or any one of the third to thirty-sixth embodiments.

In a thirty-eighth embodiment, L in the compound having the chemical structure I or II, or a pharmaceutically acceptable salt thereof, is selected from:

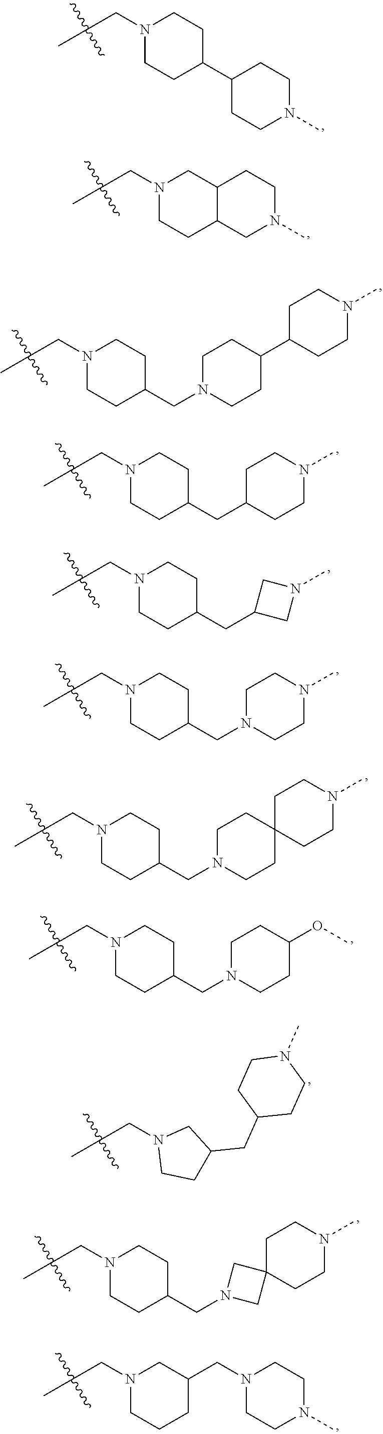

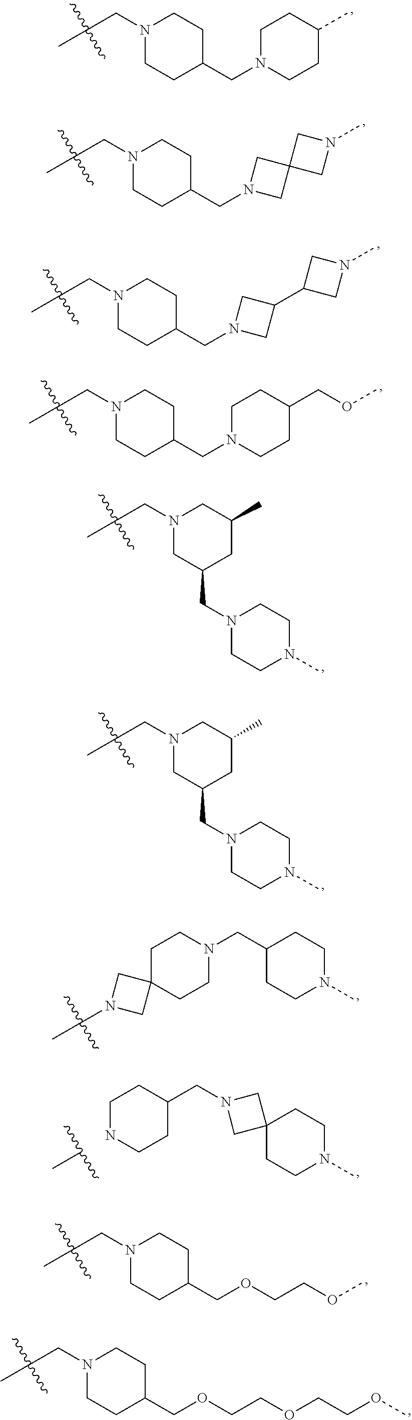

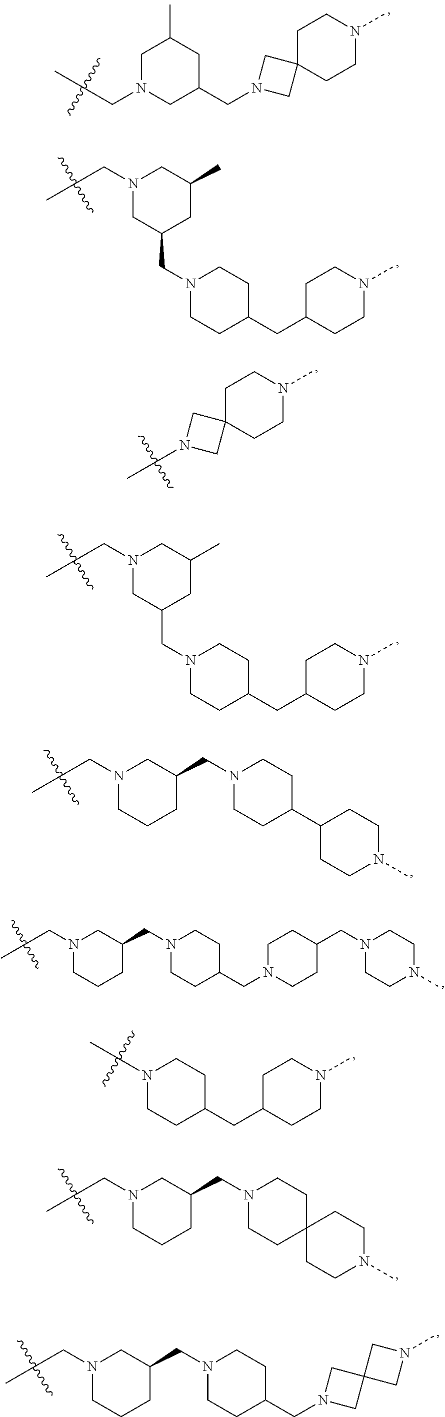

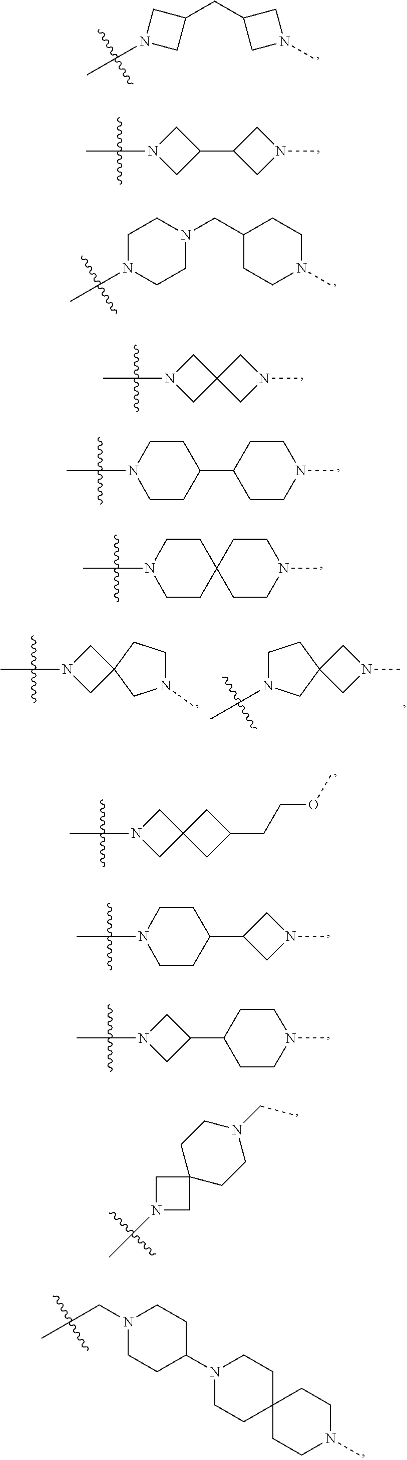

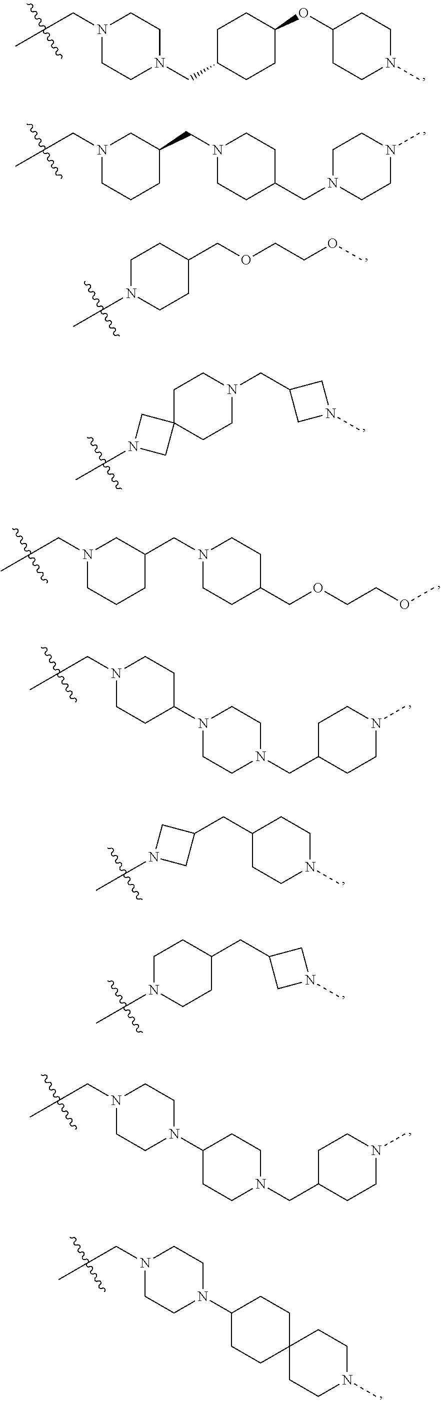

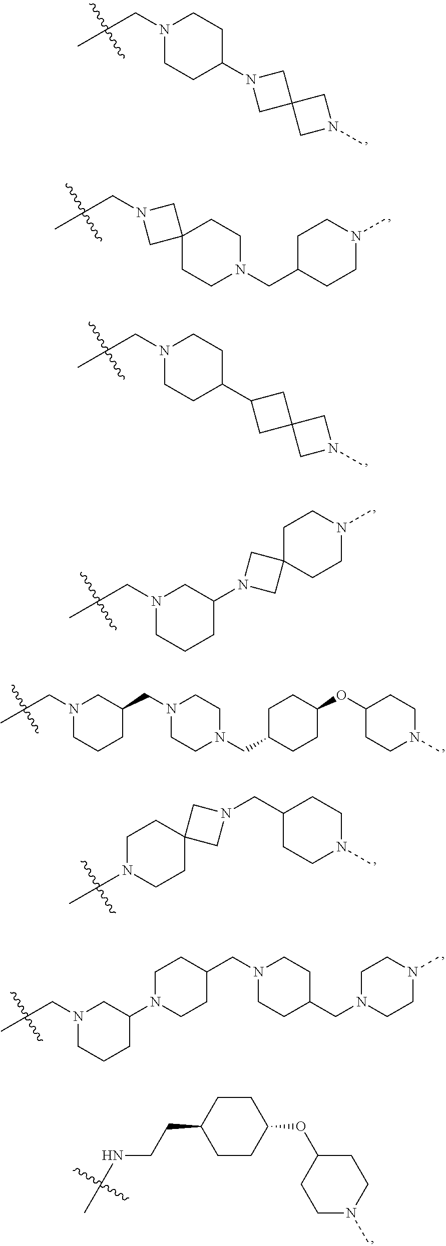

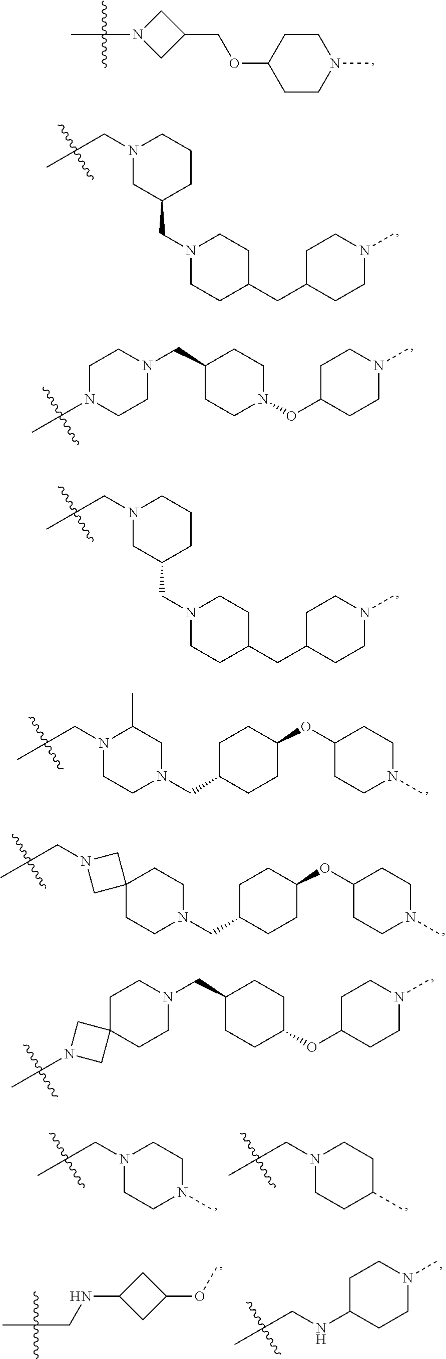

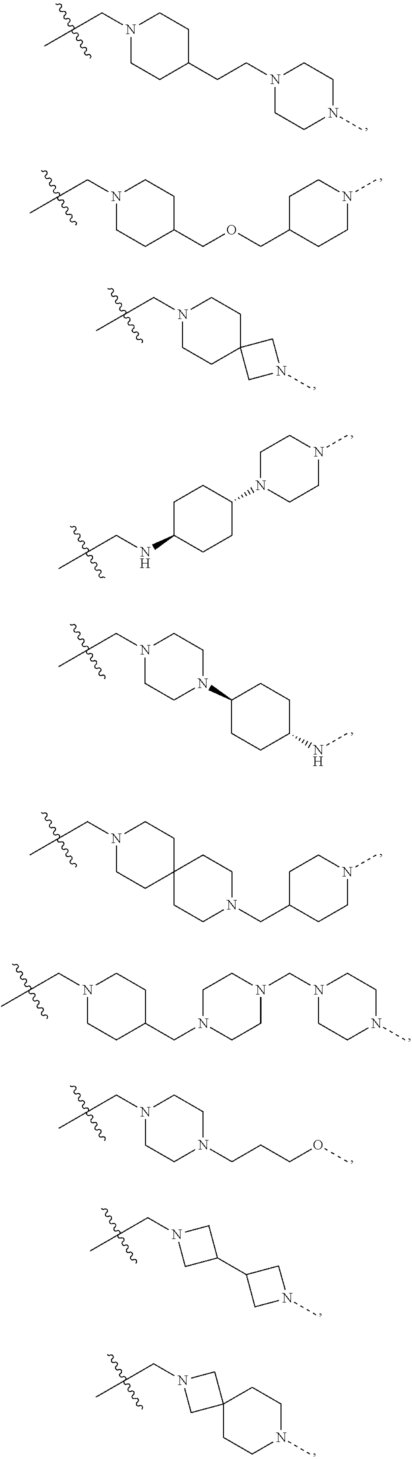

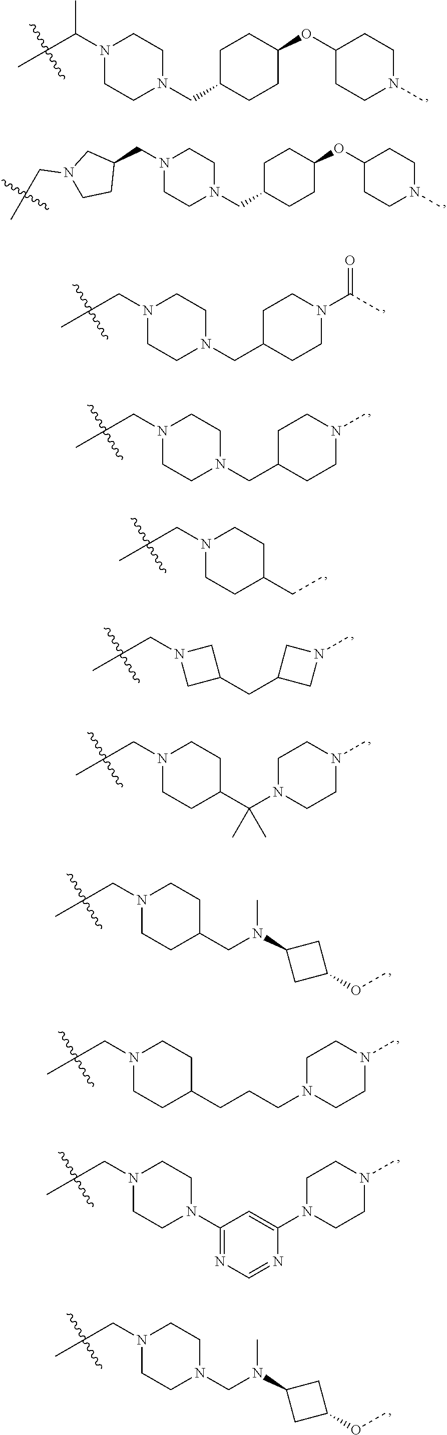

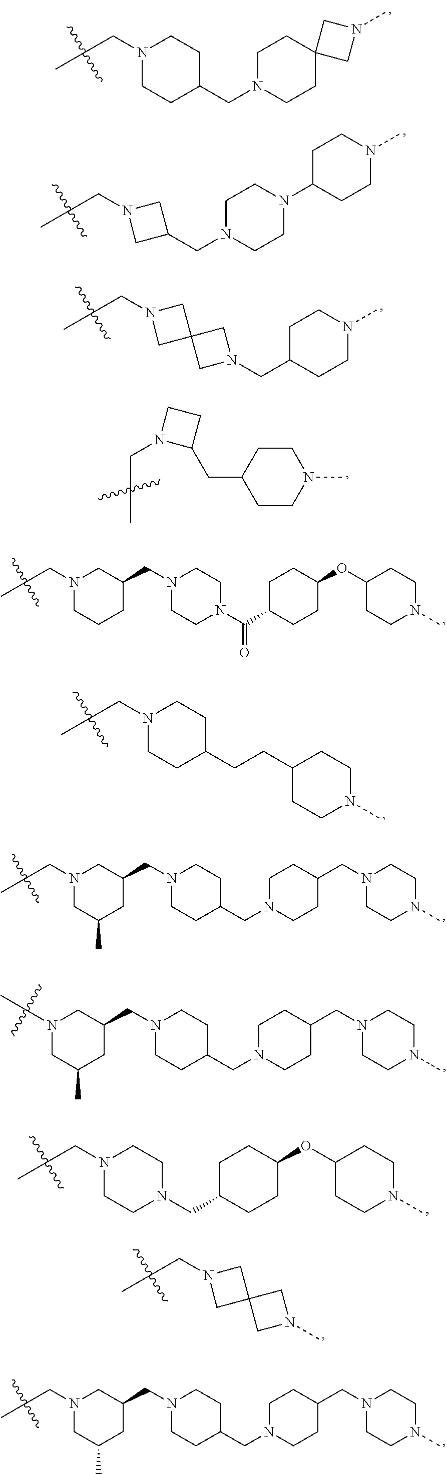

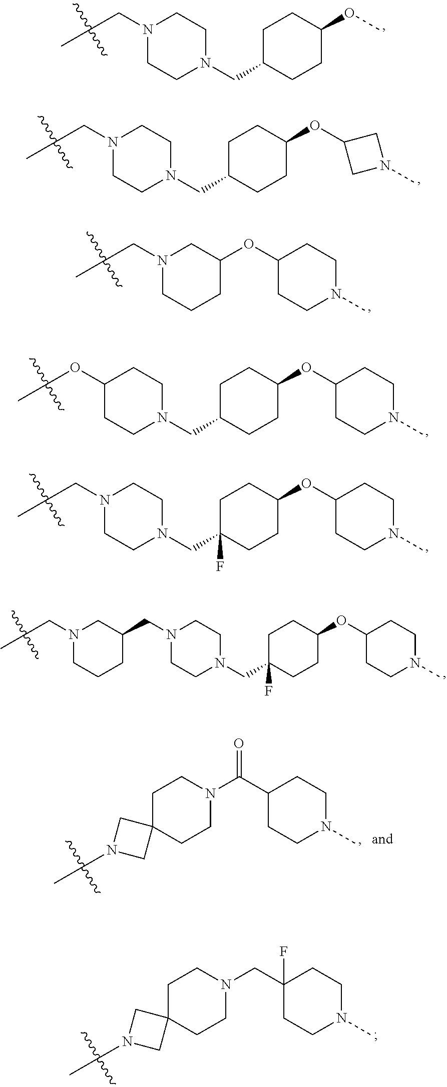

wherein the dashed bond indicates the attachment point to E wherein the remaining variables are as described above for chemical structure I or II or any one of the second to twenty-sixth embodiments. Alternatively, as part of the thirty-eighth embodiment, L in the compound having the chemical structure I or II, or a pharmaceutically acceptable salt thereof, is selected from:

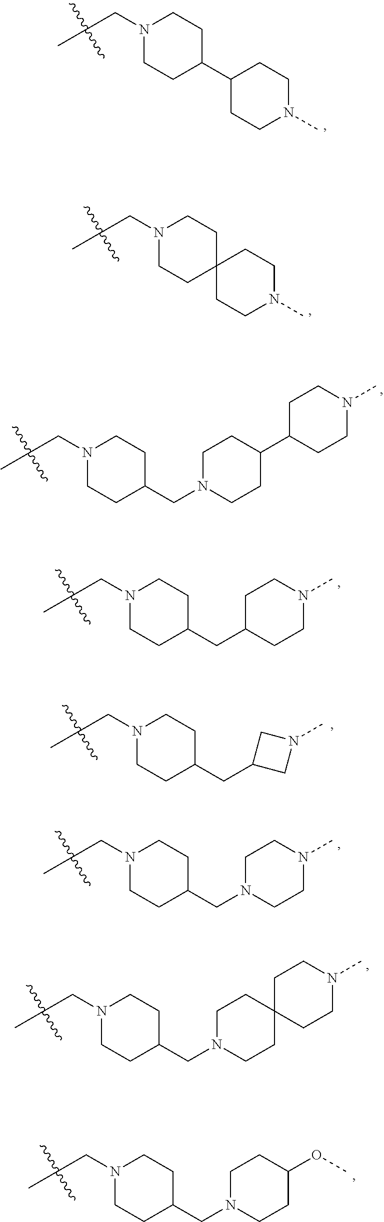

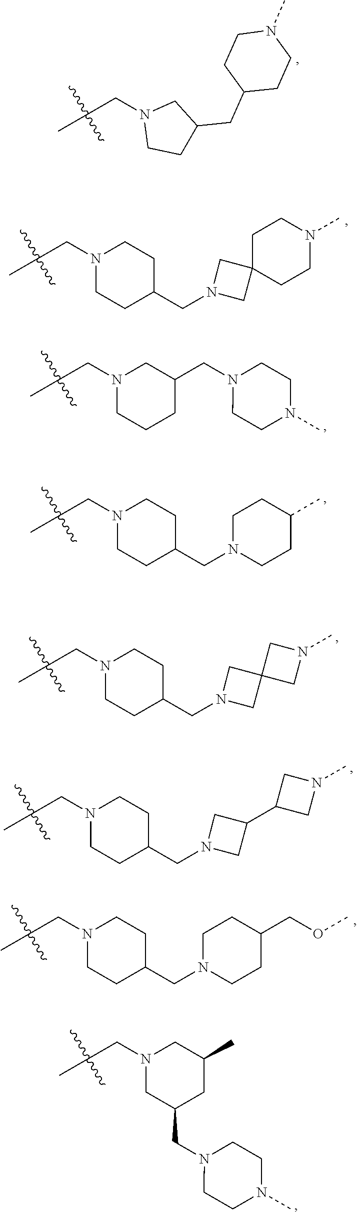

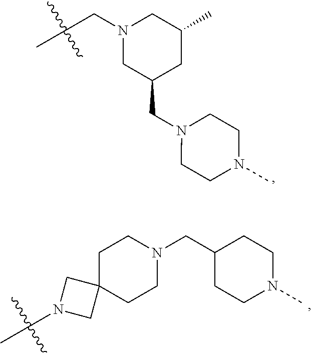

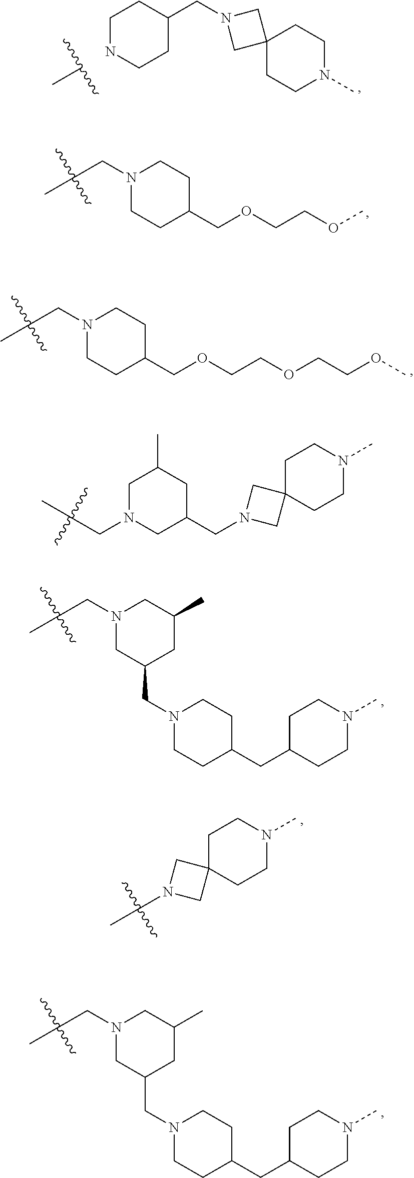

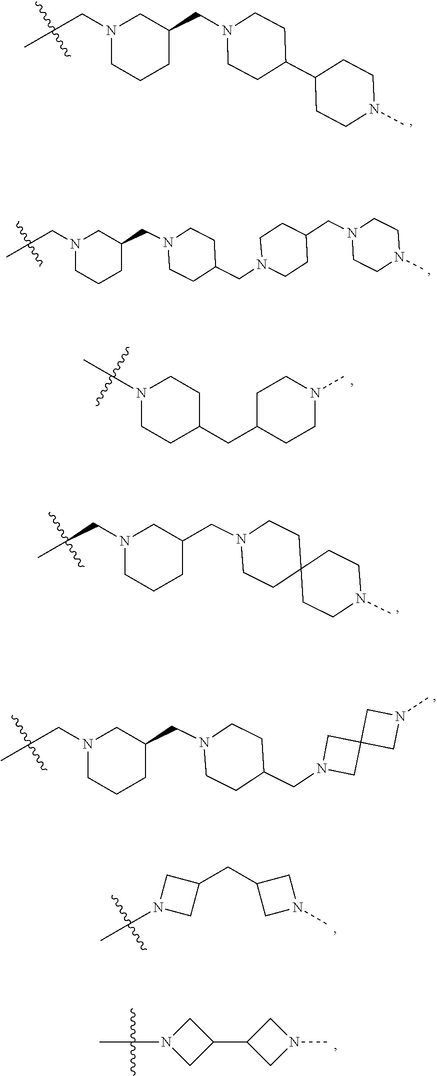

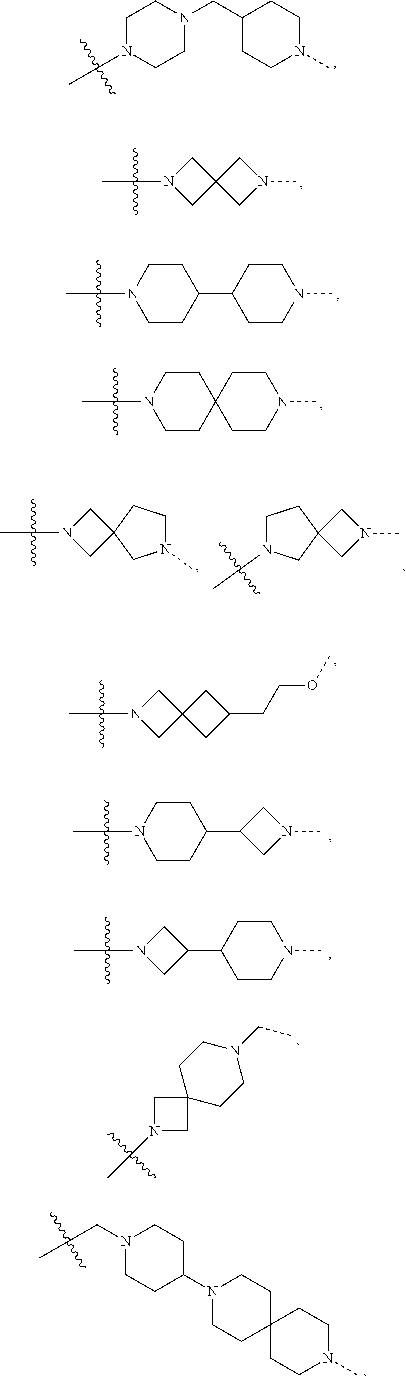

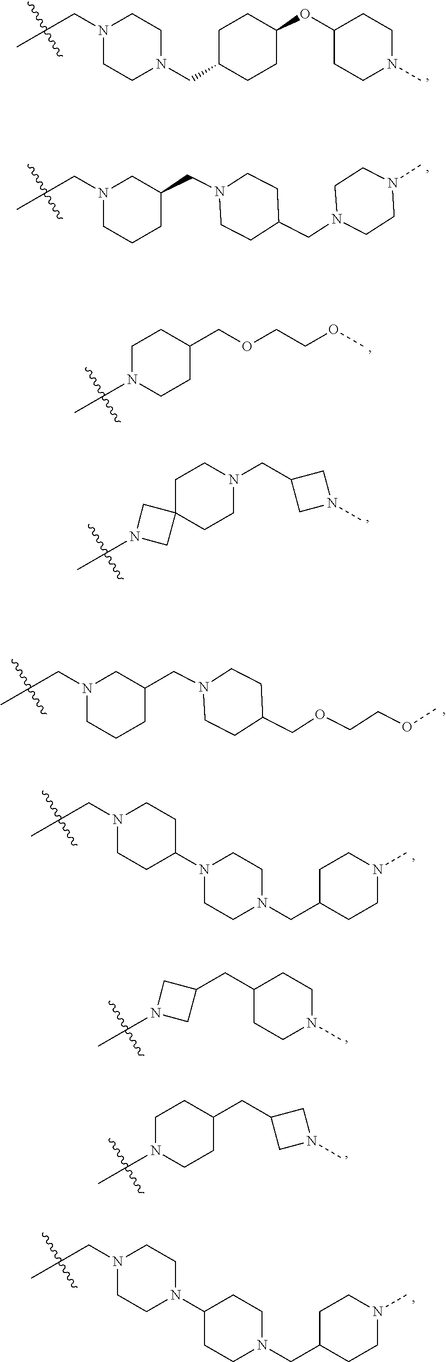

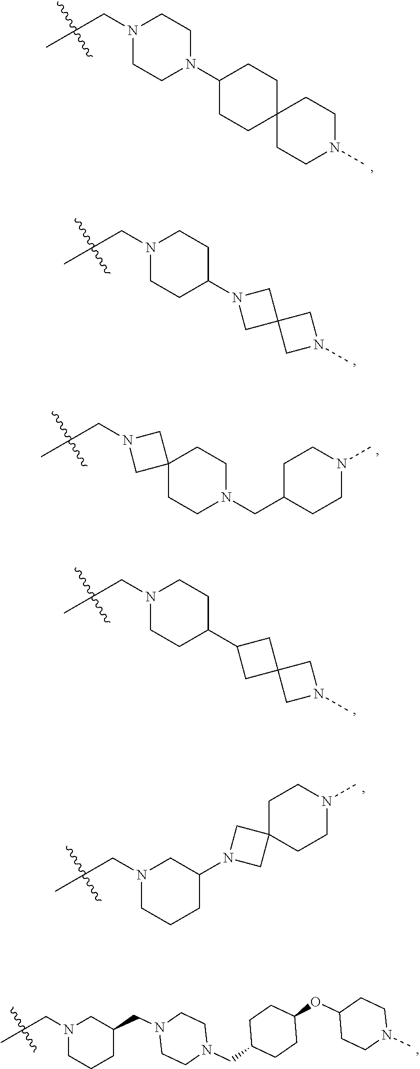

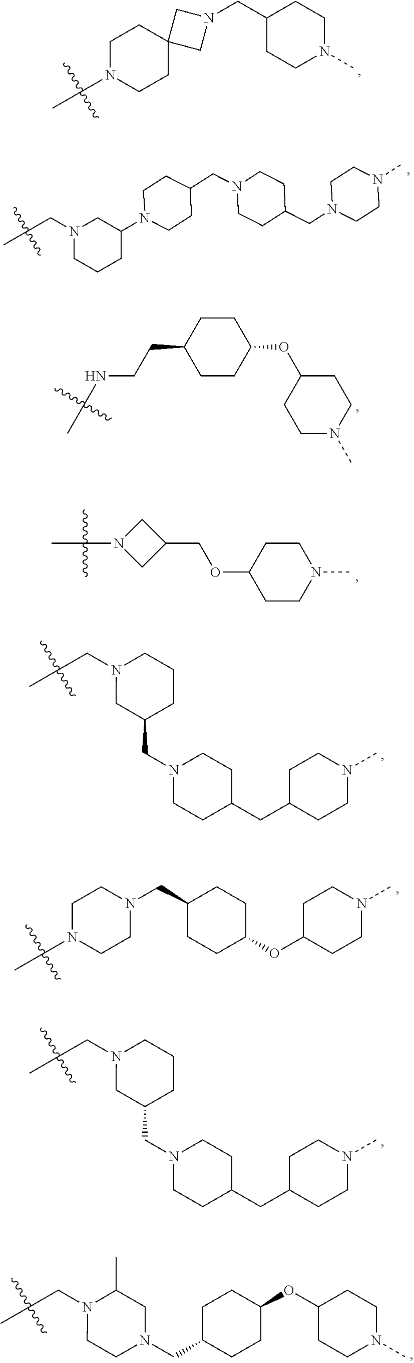

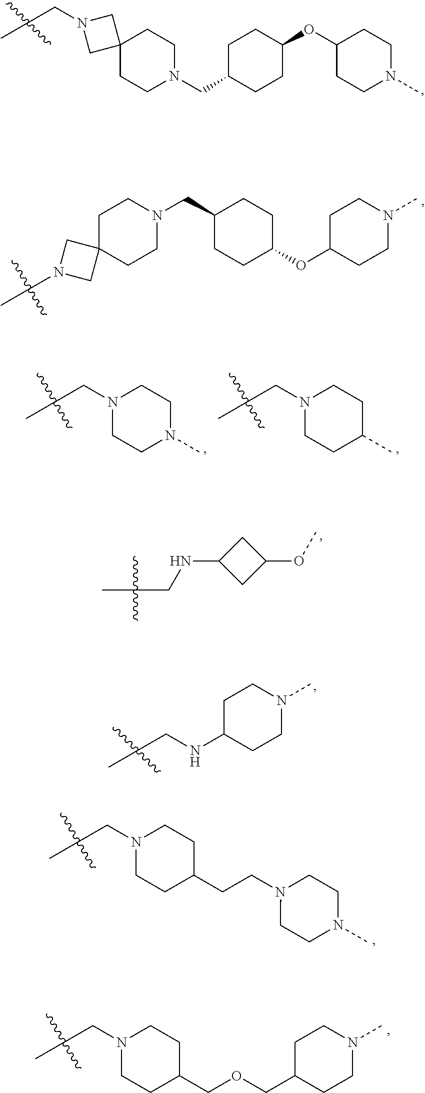

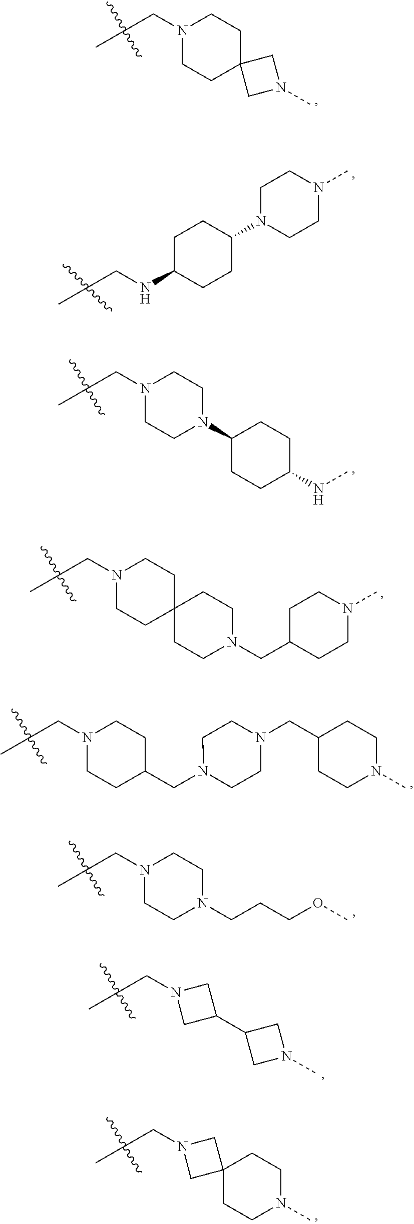

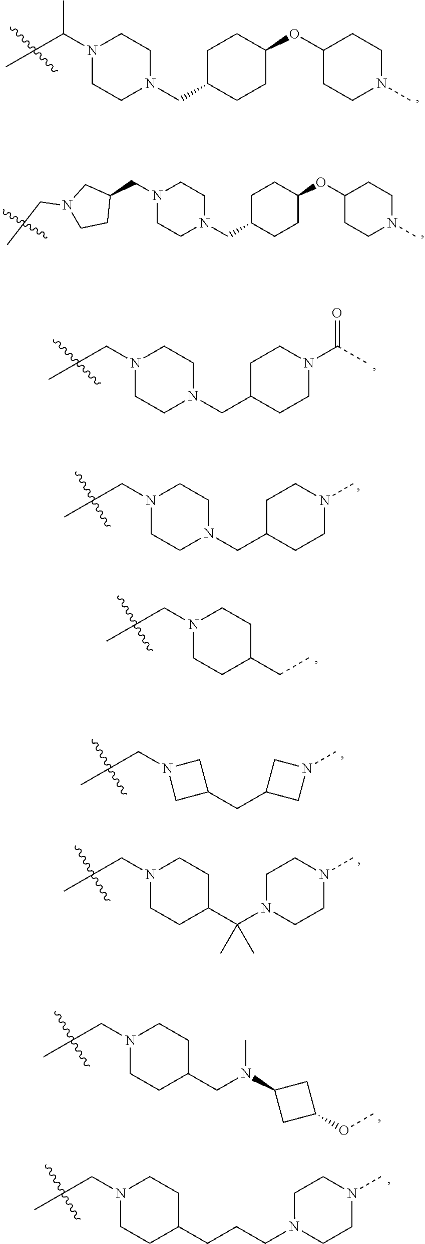

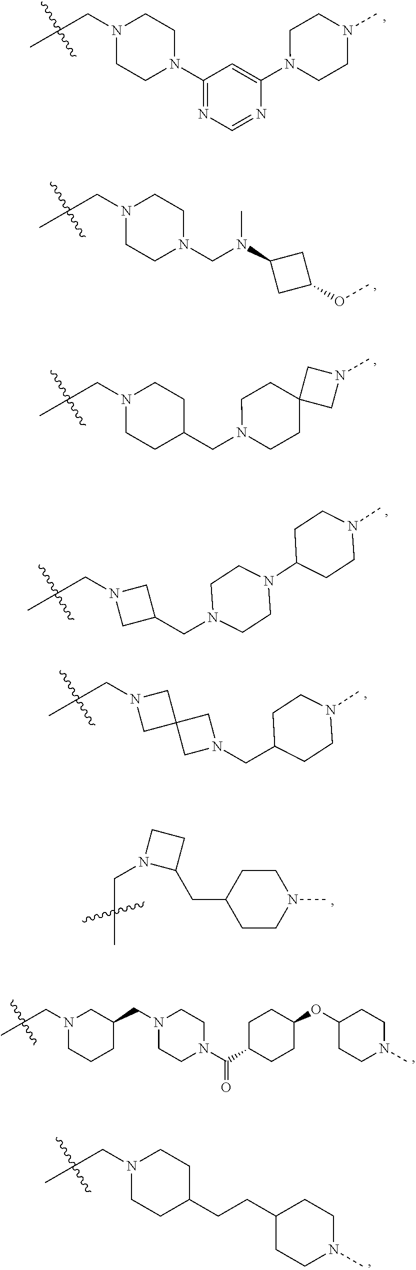

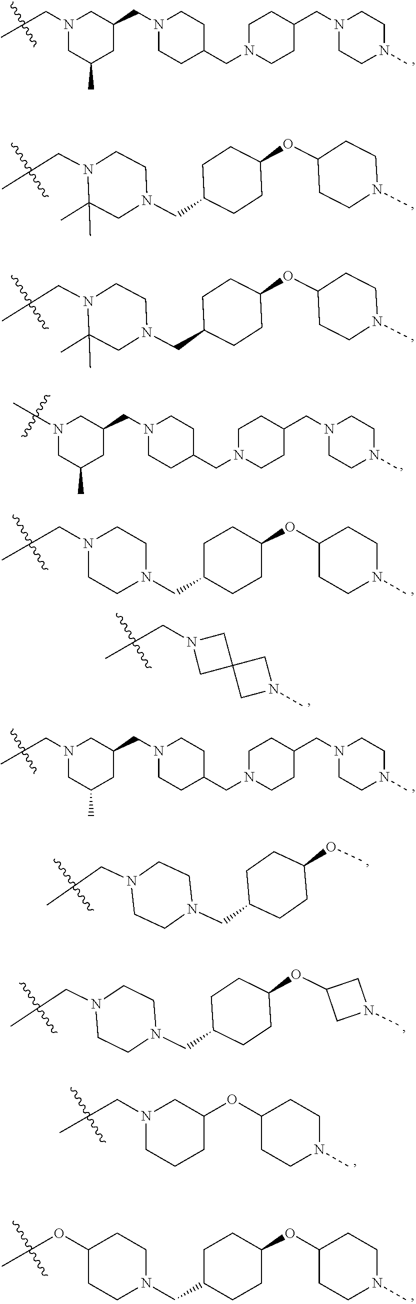

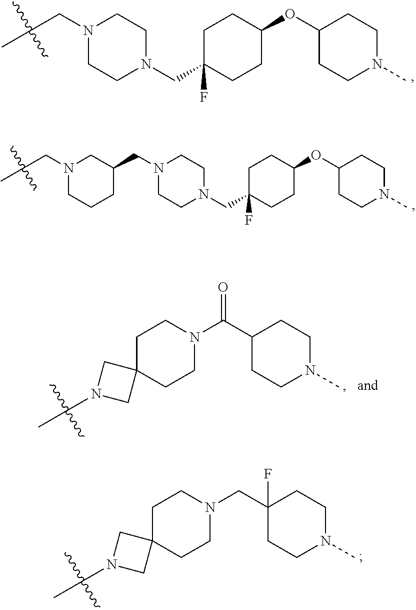

wherein the dashed bond indicates the attachment point to E wherein the remaining variables are as described above for chemical structure I or II or any one of the second to twenty-sixth embodiments. Alternatively, as part of the thirty-eighth embodiment, L in the compound having the chemical structure I or II, or a pharmaceutically acceptable salt thereof, is selected from:

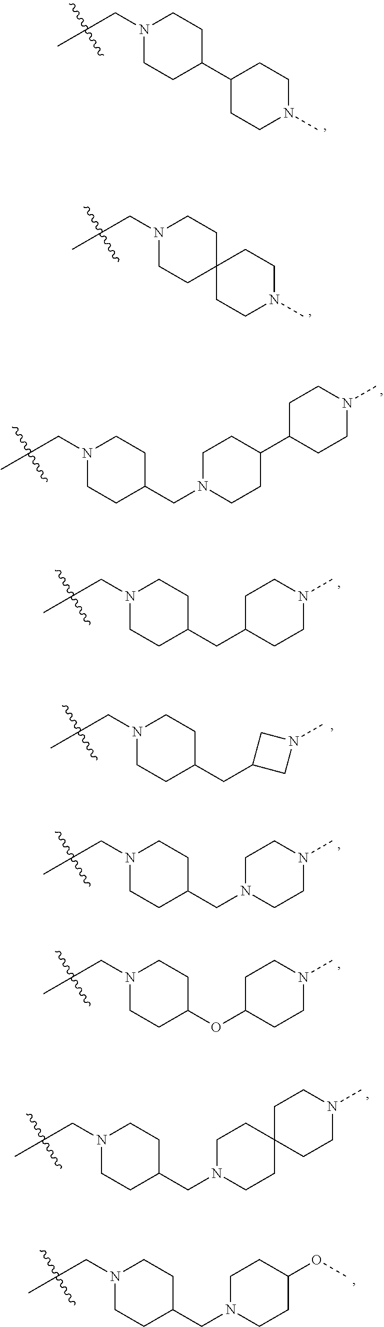

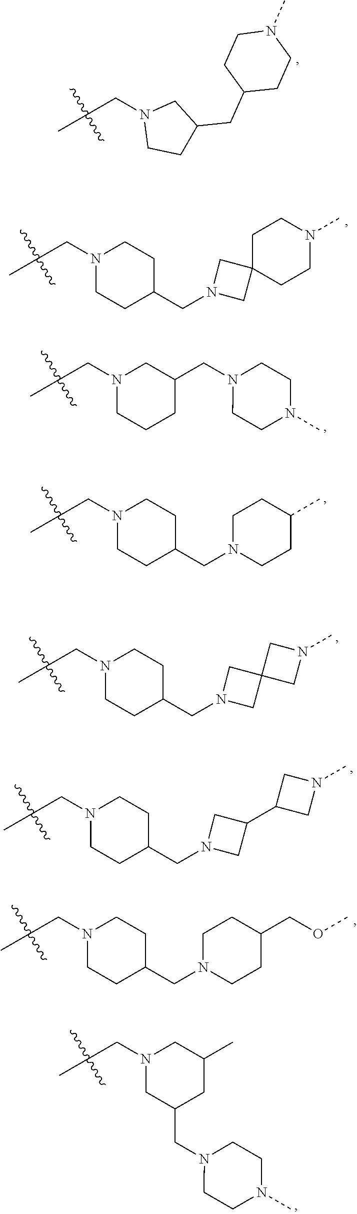

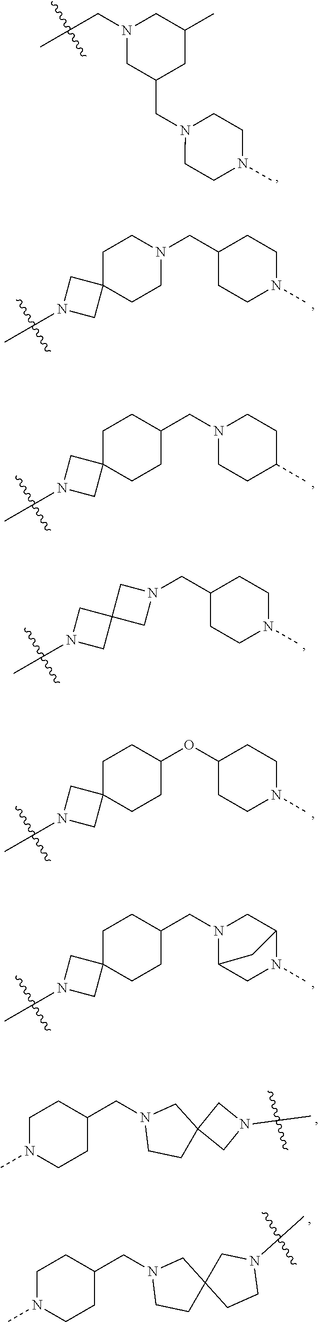

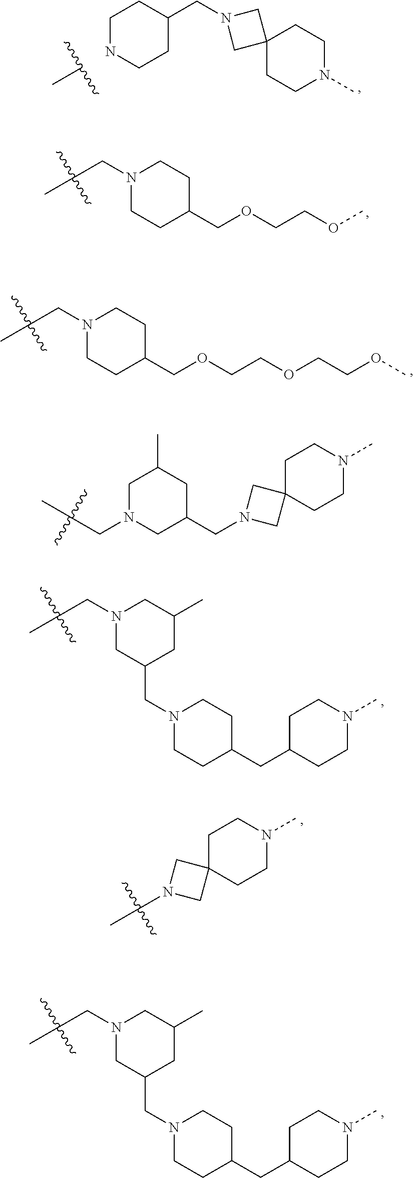

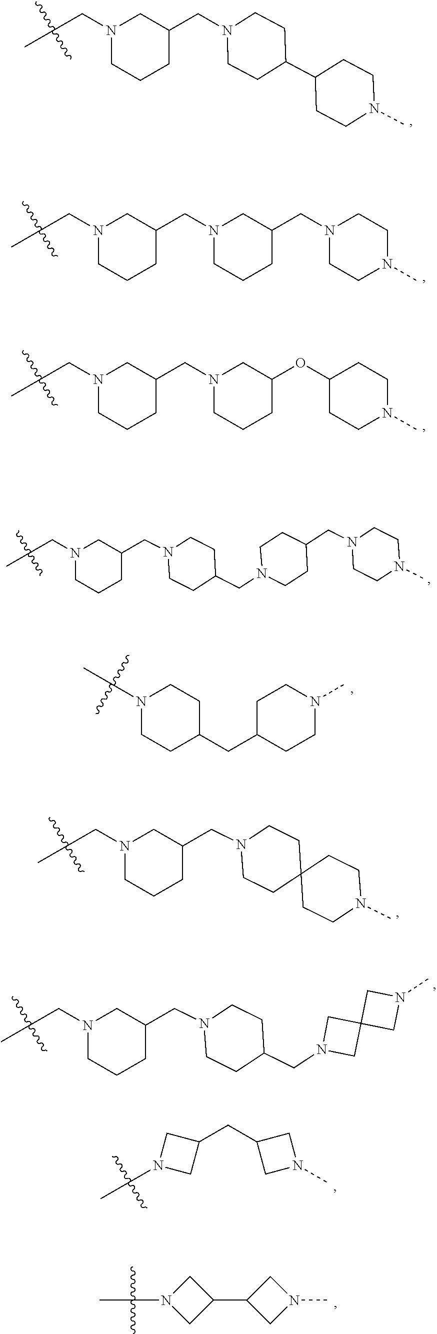

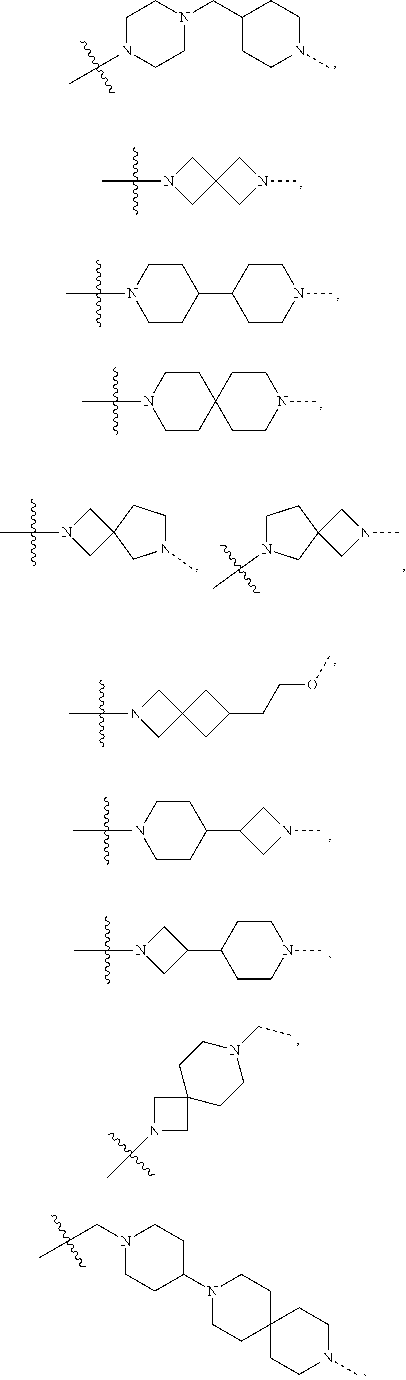

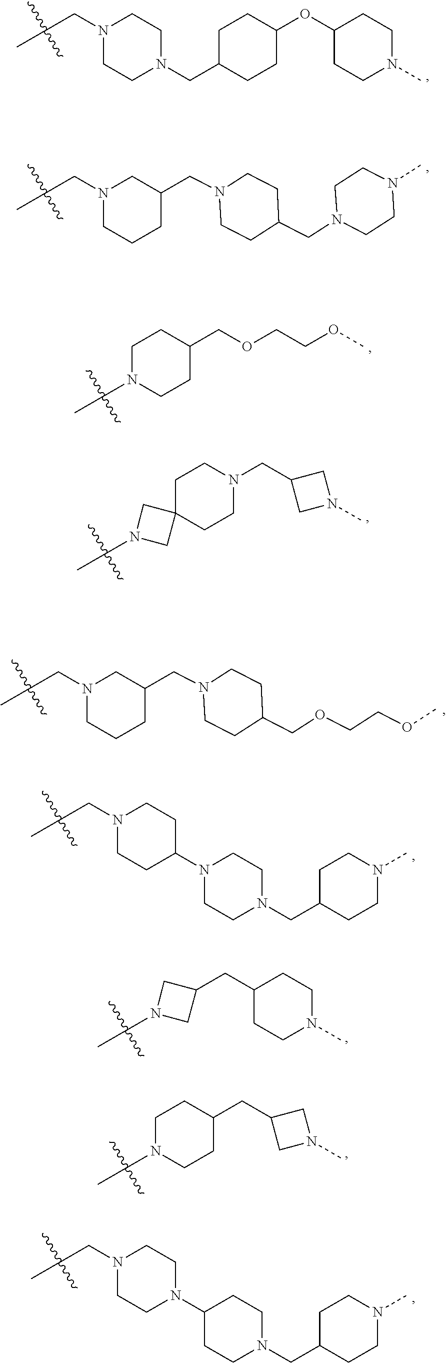

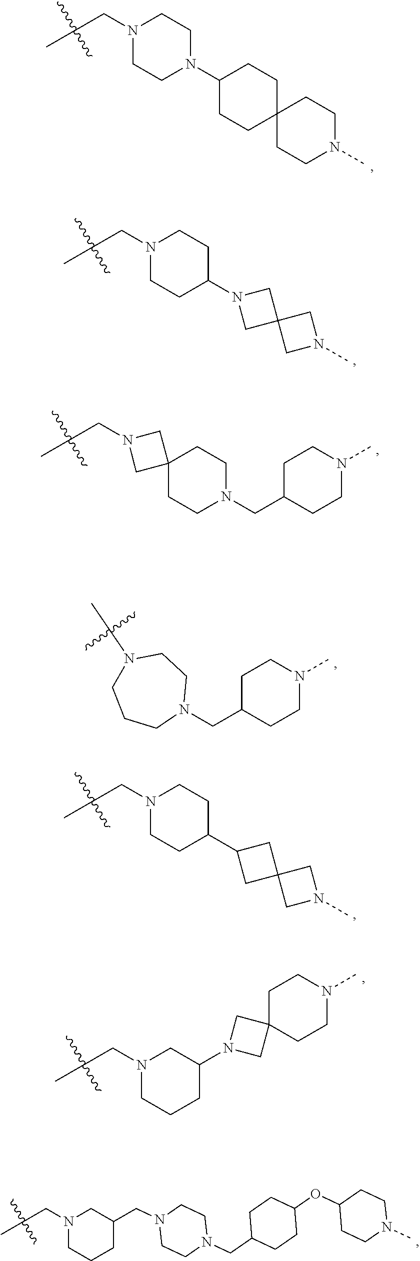

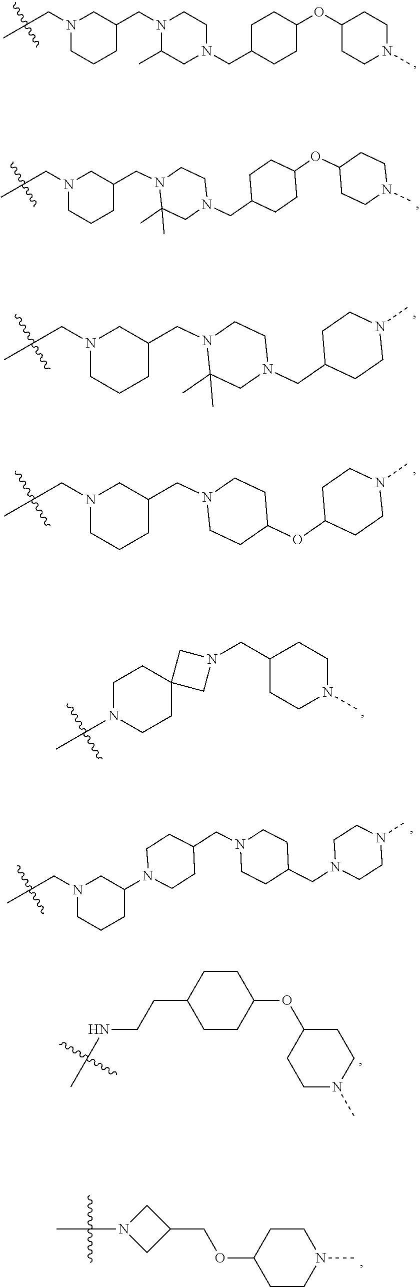

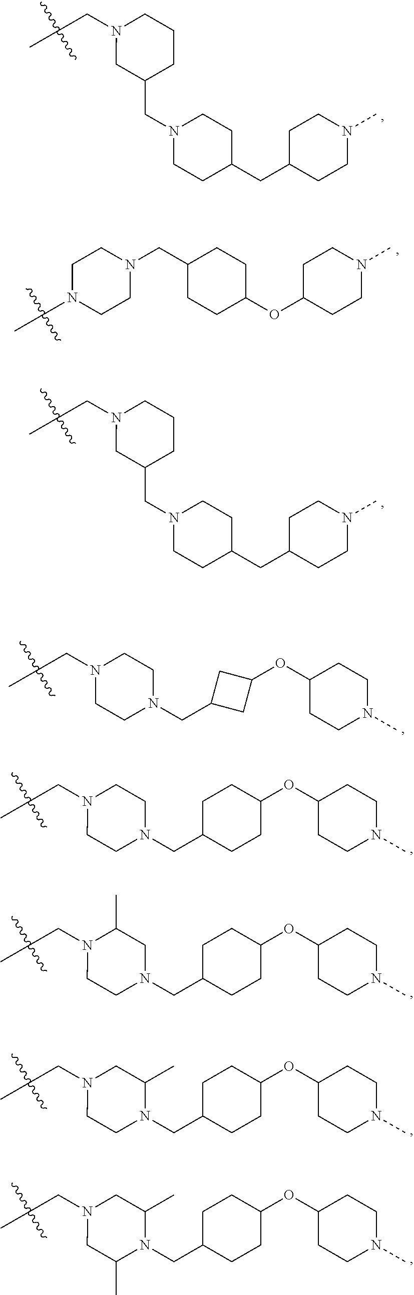

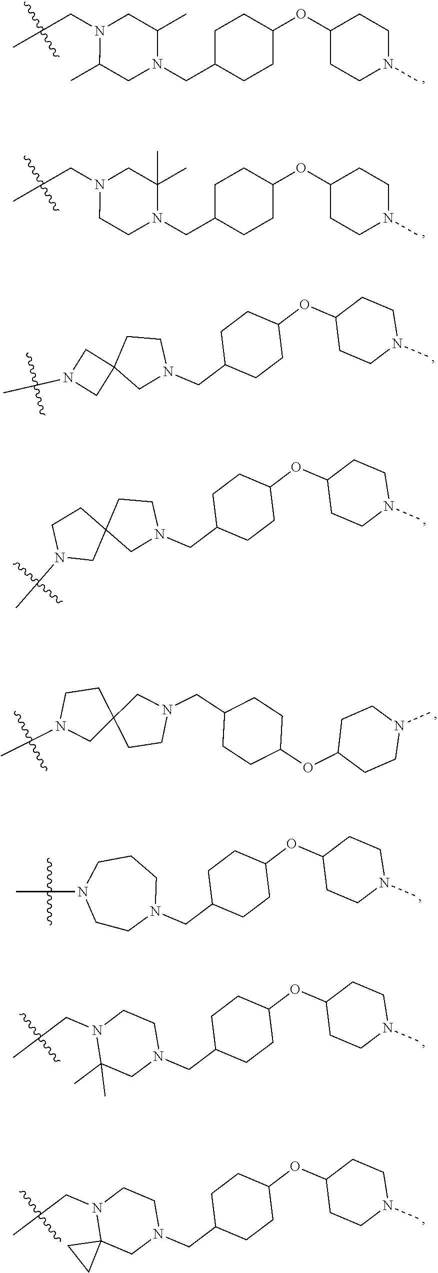

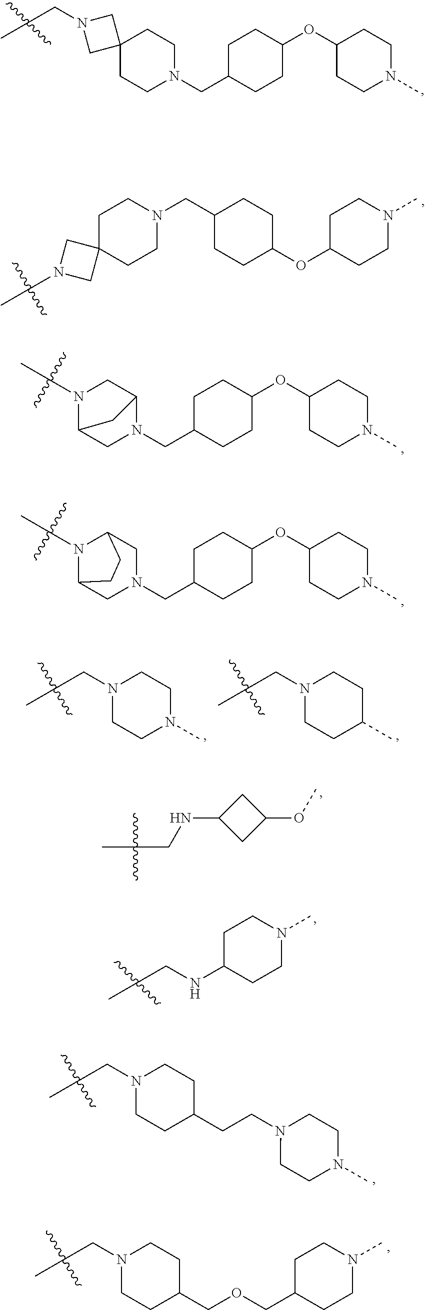

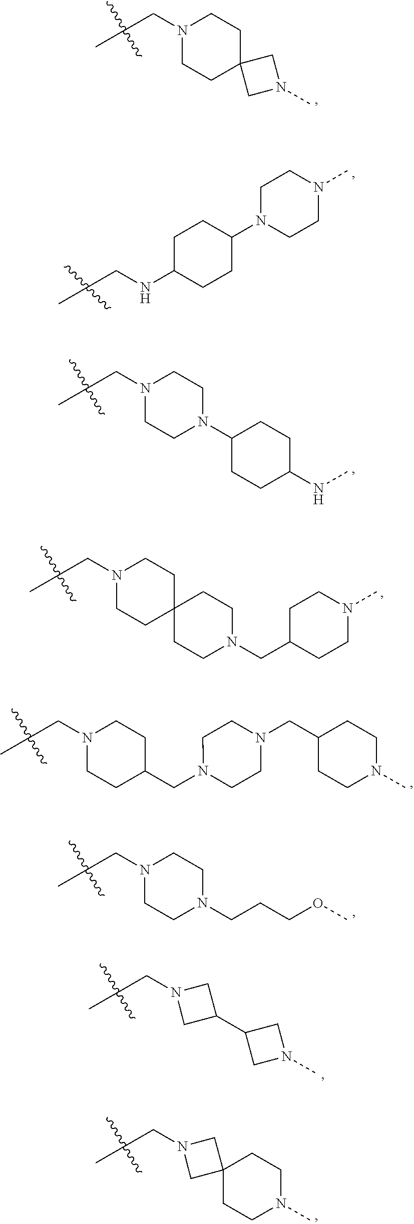

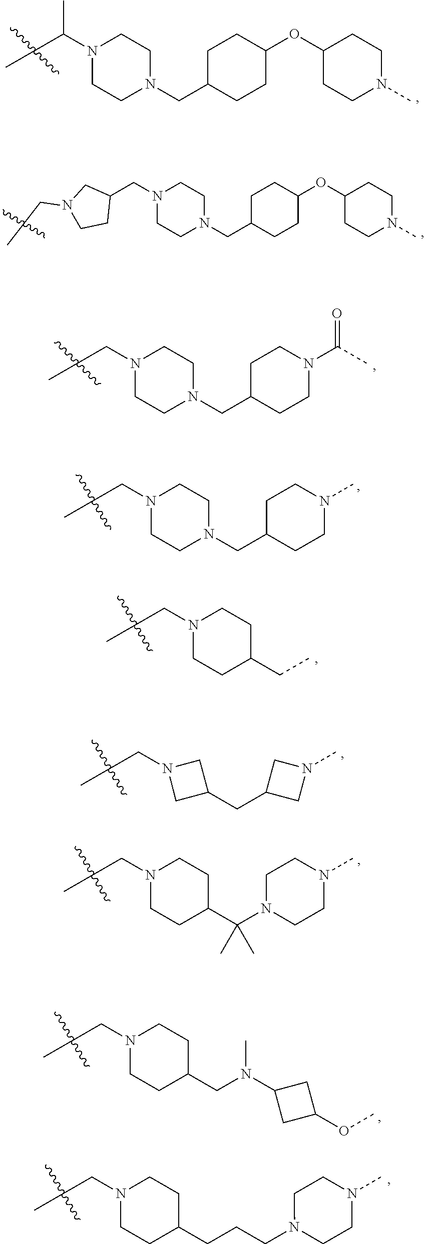

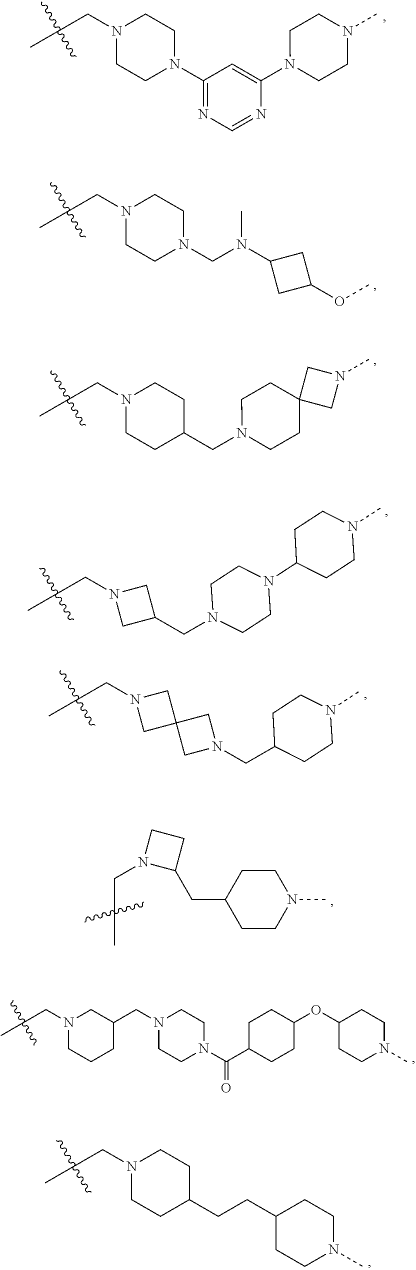

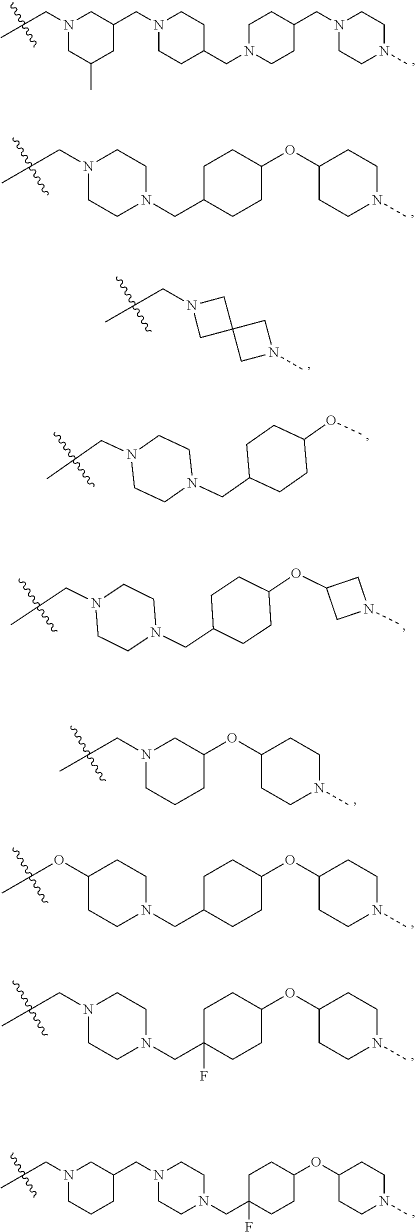

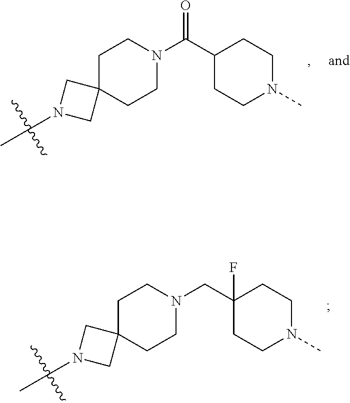

wherein the dashed bond indicates the attachment point to E wherein the remaining variables are as described above for chemical structure I or II or any one of the second to twenty-sixth embodiments. Alternatively, as part of the thirty-eighth embodiment, L in the compound having the chemical structure I or II, or a pharmaceutically acceptable salt thereof, is selected from:

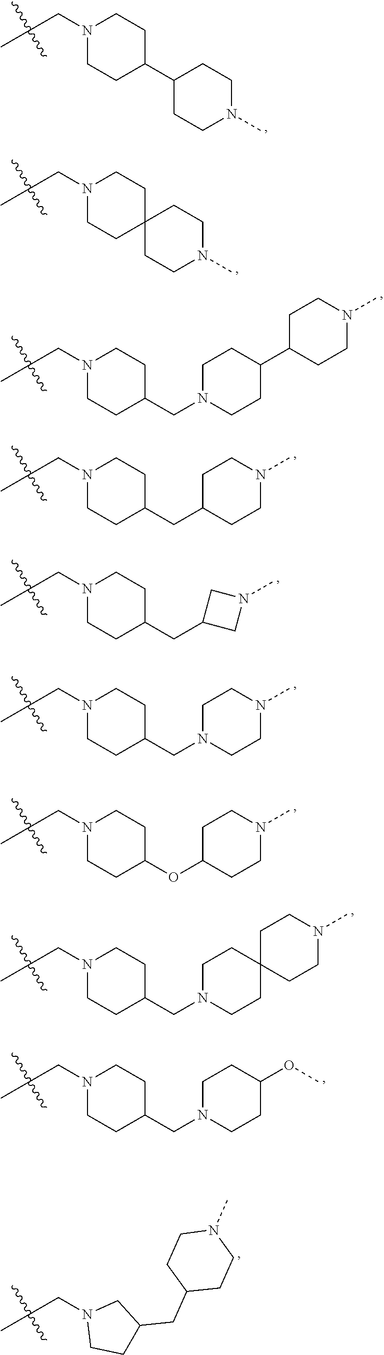

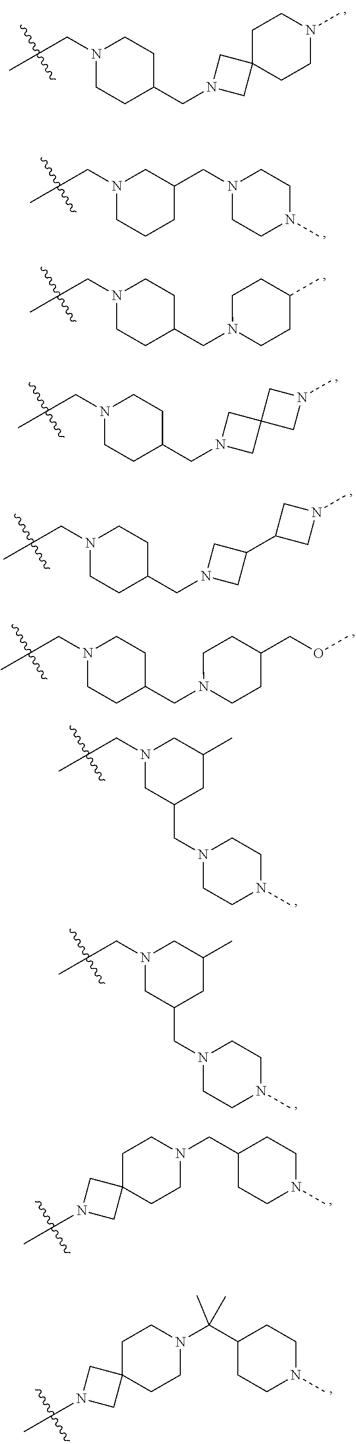

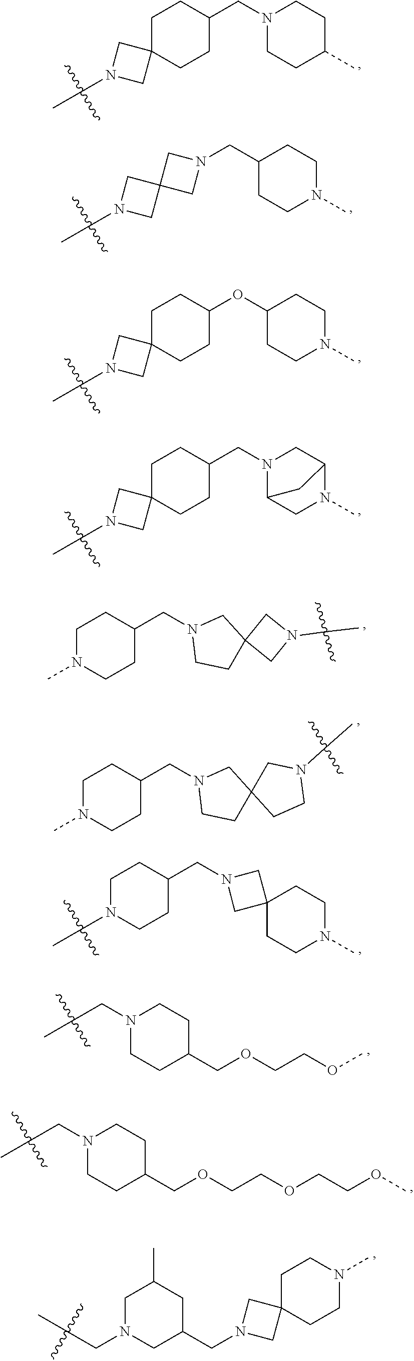

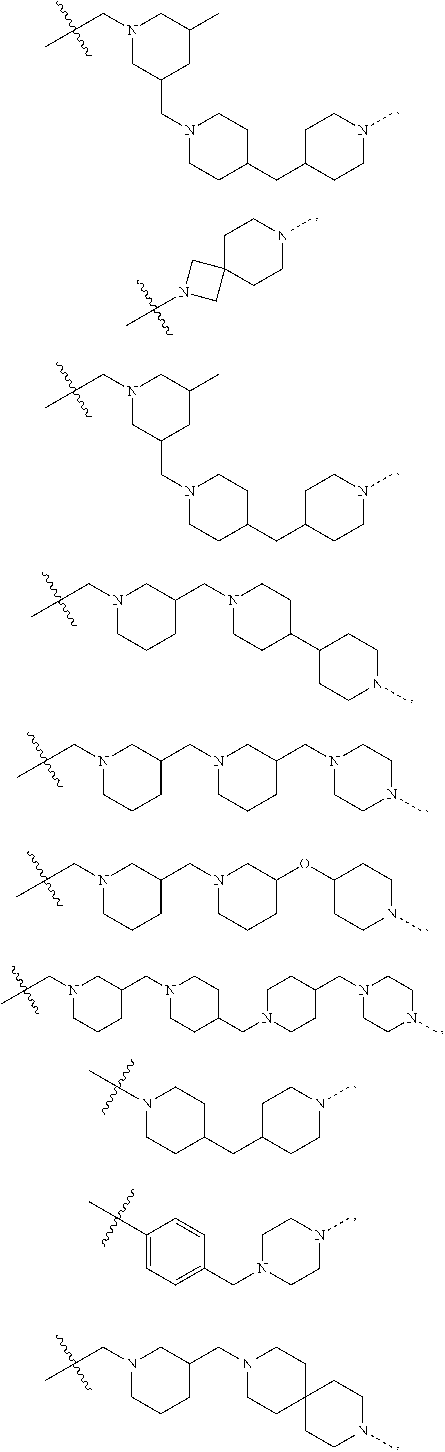

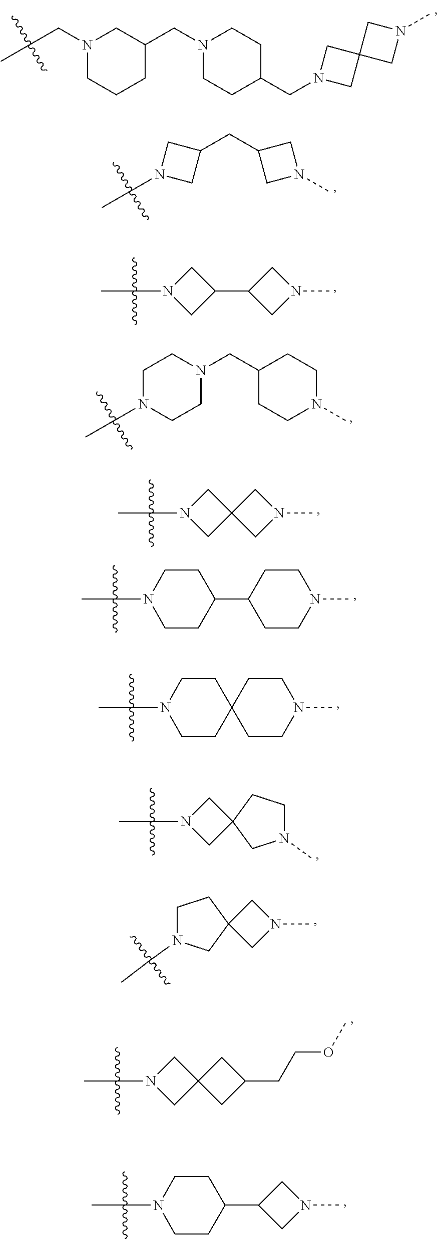

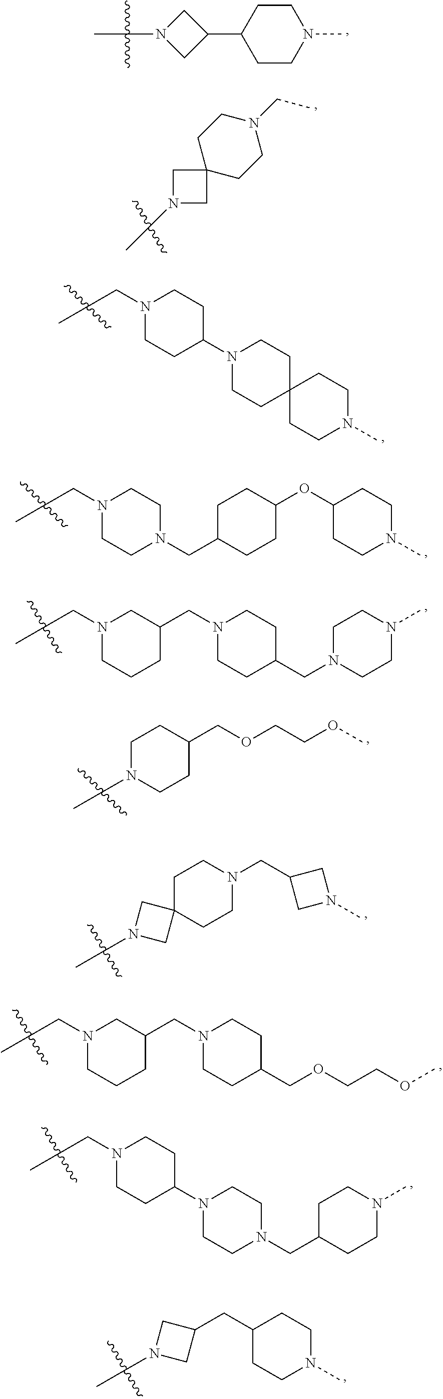

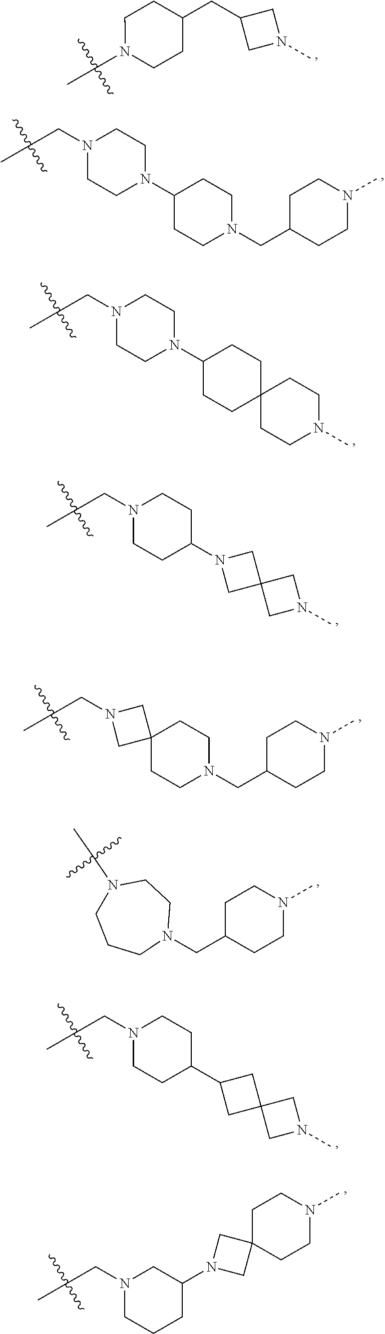

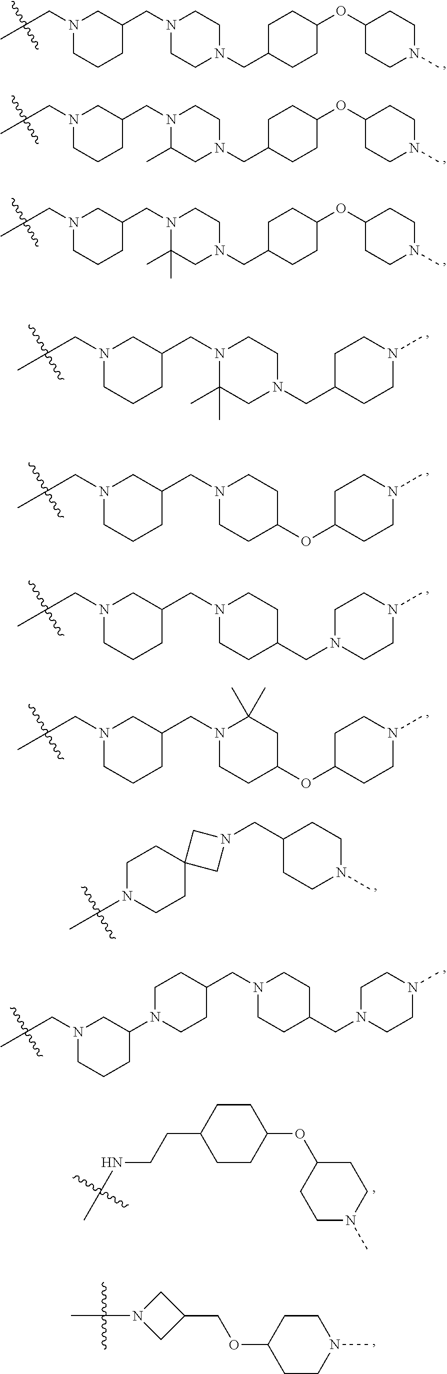

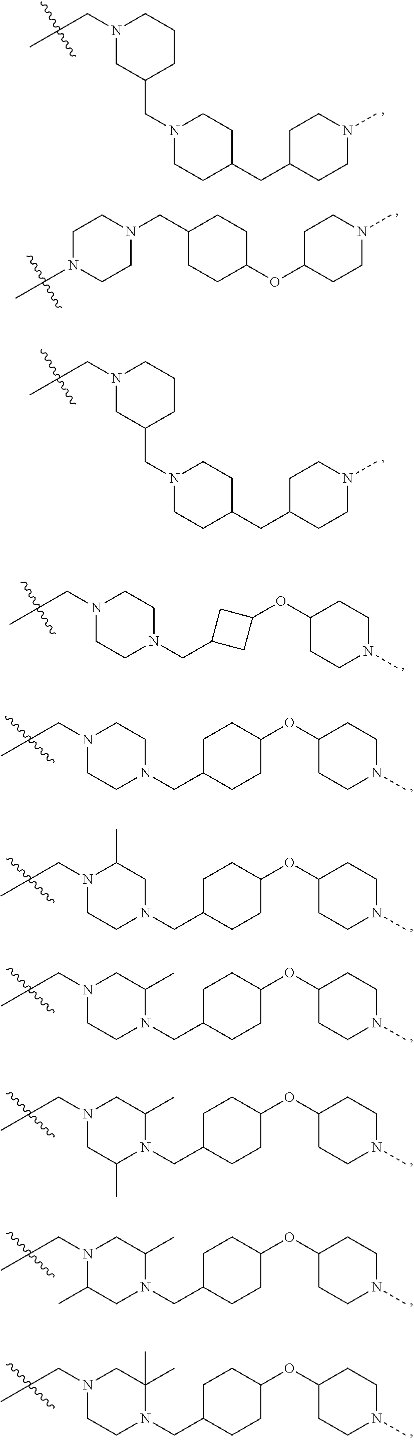

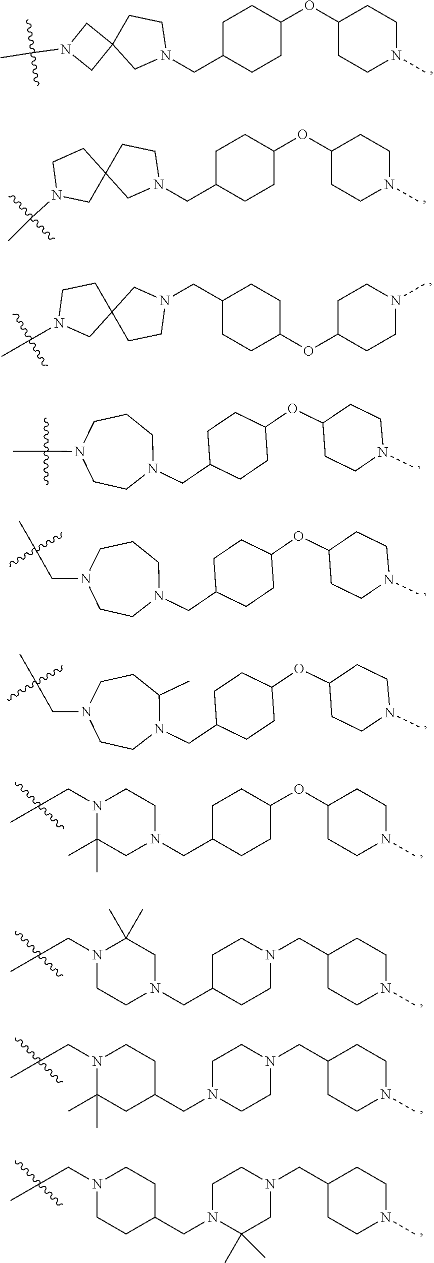

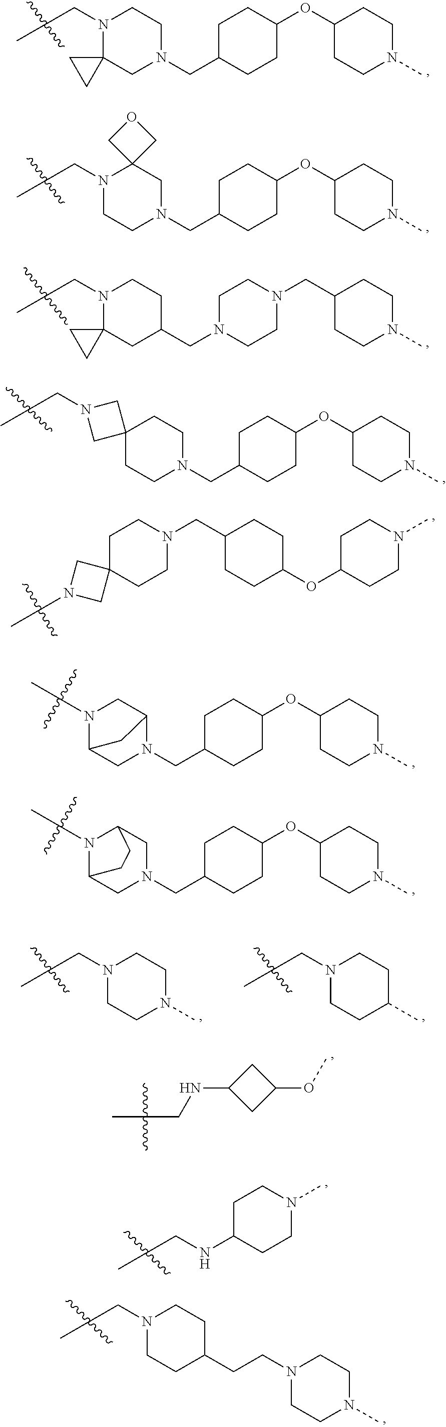

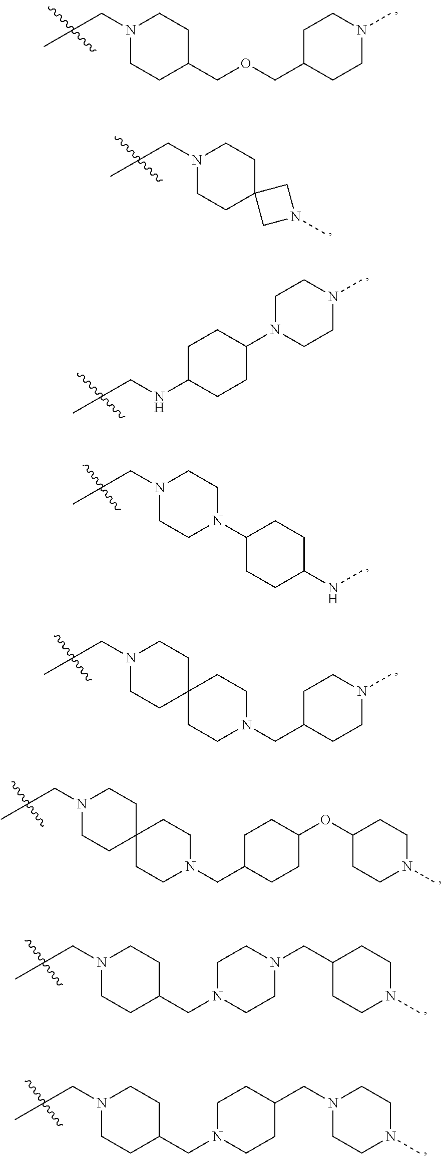

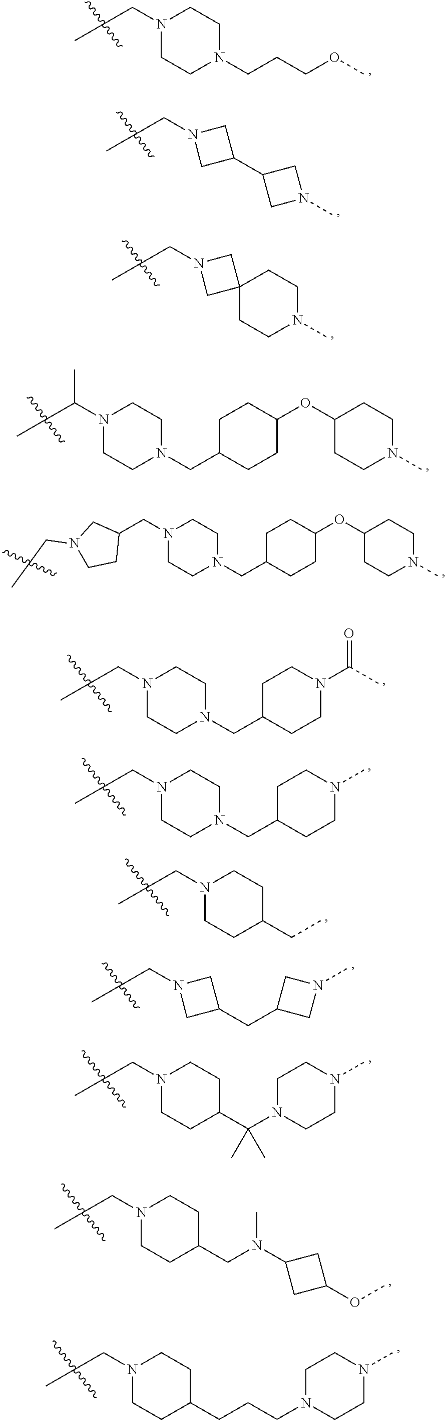

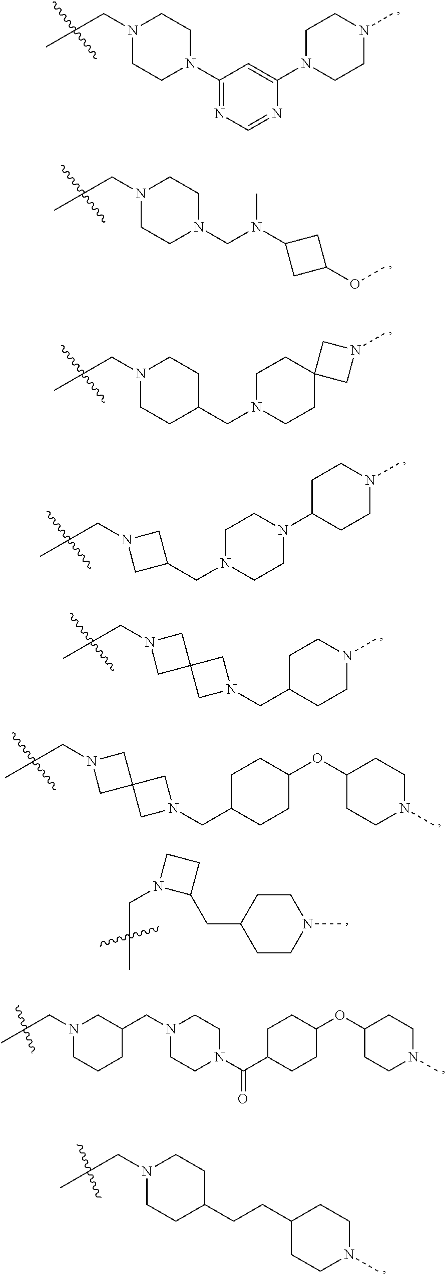

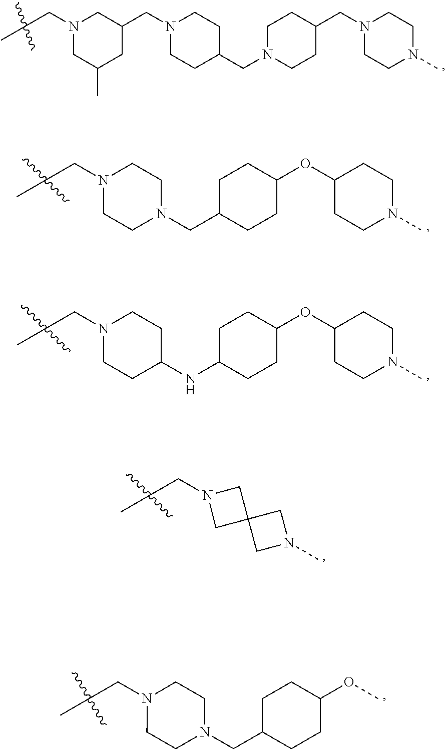

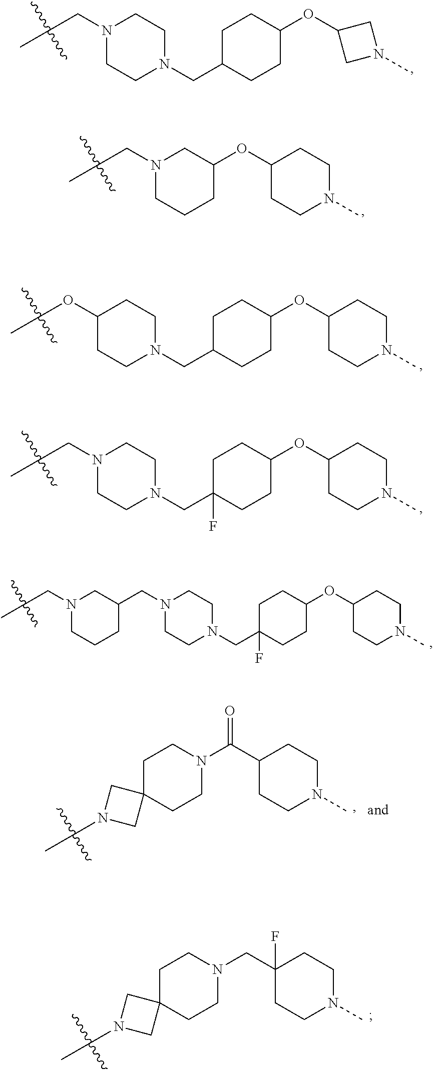

wherein the dashed bond indicates the attachment point to E wherein the remaining variables are as described above for chemical structure I or II or any one of the second to twenty-sixth embodiments.

In a thirty-ninth embodiment, the compound having the chemical structure I or II, or a pharmaceutically acceptable salt thereof, is any one of Examples 1 to 328.

Compounds having the chemical structure I or II are further described in the Exemplification and are included in the present disclosure. Pharmaceutically acceptable salts thereof as well as the neutral forms of the compounds described herein are also included.

Also provided herein are pharmaceutical compositions comprising a described compound or a pharmaceutically acceptable salt of a described compound, or pharmaceutical compositions comprising a described compound or a pharmaceutically acceptable salt of a compound described herein; and a pharmaceutically acceptable carrier.

### 4. Uses and Administration

The compounds and compositions described herein are generally useful for treating a Cbl-b related condition. Thus, in one aspect, provided are methods of treating a Cbl-b related condition in a subject in need thereof comprising administering to the subject a therapeutically effective amount of a compound described herein, or a pharmaceutically acceptable salt thereof, or a pharmaceutical composition comprising a disclosed compound or pharmaceutically acceptable salt thereof.

Also provided is the use of a compound described herein, or a pharmaceutically acceptable salt thereof, or a pharmaceutical composition comprising a disclosed compound or pharmaceutically acceptable salt thereof, for the manufacture of a medicament for treating a Cbl-b related condition. Also provided is a compound described herein, or a pharmaceutically acceptable salt thereof, or a pharmaceutical composition comprising a disclosed compound or pharmaceutically acceptable salt thereof, for use in treating a Cbl-b related condition.

In one aspect, the Cbl-b related conditions are those which are modulated by degrading the Cbl-b protein.

In certain aspects, the Cbl-b related condition is cancer. In certain aspect the cancer is selected from melanoma, lung cancer, head and neck cancer, prostate cancer, colorectal cancer, or renal cancer. In certain aspects the cancer is selected from melanoma, lung cancer, head and neck cancer, prostate cancer, colorectal cancer, renal cancer, urothelial cancer, bladder cancer, hepatocellular carcinoma, pancreatic cancer, breast cancer, or hematology cancer. In certain aspects, the non-small cell lung cancer, Squamous cell carcinoma of the head and neck, metastatic castration resistant prostate cancer, or MSS colorectal cancer. In certain aspects, the cancer is susceptible to CBLb induced tumor immunity suppression.

The compounds, the pharmaceutically acceptable salts of the compounds, and the pharmaceutical compositions described herein may be administered orally, parenterally, by inhalation spray, topically, rectally, nasally, buccally, vaginally or via an implanted reservoir. Preparations for such pharmaceutical compositions are well-known in the art. See, e.g., Anderson, Philip O.; Knoben, James E.; Troutman, William G., eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2003; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins, 2000; Martindale, The Extra Pharmacopoeia, Thirty-Second Edition (The Pharmaceutical Press, London, 1999); all of which are incorporated by reference herein in their entirety. The term “parenteral” as used herein includes subcutaneous, intravenous, intramuscular, intra-articular, intra-synovial, intrasternal, intrathecal, intrahepatic, intralesional and intracranial injection or infusion techniques. Liquid dosage forms, injectable preparations, solid dispersion forms, and dosage forms for topical or transdermal administration of the compounds, the pharmaceutically acceptable salts of the compounds, and the pharmaceutical compositions described herein are included herein. In one aspect, the compounds, the pharmaceutically acceptable salts of the compounds, and the pharmaceutical compositions described herein are administered orally.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the pharmaceutical composition.

### EXAMPLES

### General Synthetic Approach

General synthetic schemes for preparing the described compounds are described below. These schemes are illustrative and are not meant to limit the possible techniques one skilled in the art may use to prepare the compounds described herein.

Starting materials and reagents used in these examples, when not prepared by a procedure described herein, are generally either commercially available, or are reported in the chemical literature, or may be prepared by using procedures described in the chemical literature.

In some instances, protecting group strategies and/or functional group interconversions (FGIs) may be required to facilitate the preparation of the desired materials. Such chemical processes are well known to the synthetic organic chemist and many of these may be found in texts such as “Greene's Protective Groups in Organic Synthesis” Peter G. M. Wuts and Theodora W. Greene (Wiley), and “Organic Synthesis: The Disconnection Approach” Stuart Warren and Paul Wyatt (Wiley).

### Synthetic Procedures

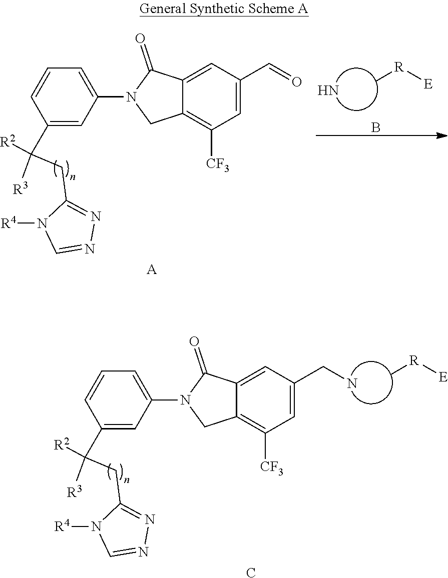

A compound of formula A may be reacted with a compound of formula B to afford a compound of formula C under conditions suitable for a reductive amination reaction, e.g. sodium triacetoxyborohydride and triethylamine or diisopropylethylamine; or sodium cyanoborohydride, sodium acetate, and acetic acid; in a suitable solvent such as isopropanol/dichloromethane, dimethylsulfoxide/dichloromethane, dichloromethane, dichloroethane, or dimethylsulfoxide/dichloroethane at room temperature. As needed, mixtures of enantiomers or diastereomers of the compounds may be resolved into their constituent enantiomers or diastereomers using techniques known to one skilled in the art, including but not limited to preparative high performance liquid chromatography or preparative supercritical fluid chromatography.

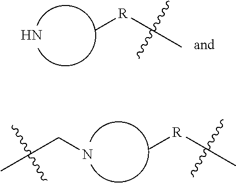

each represent a portion of linker (L) as defined herein where

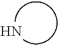

is a primary or secondary amine, optionally cyclized into a heterocyclic ring. All other variables are as defined herein.

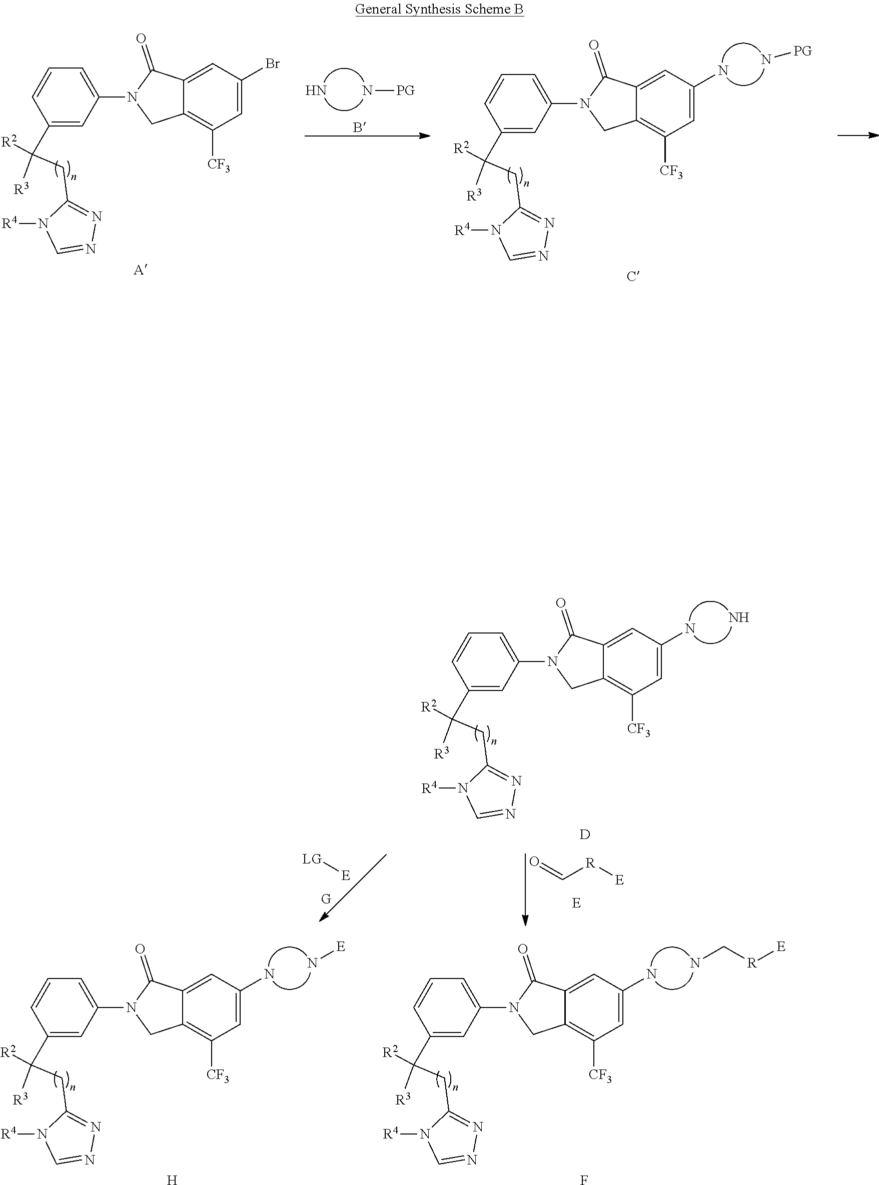

A compound of formula A′ may be reacted with a compound of formula B′ to afford a compound of formula C under conditions suitable for an aryl amination reaction, e.g. Pd(OAc)₂ or other suitable palladium catalyst, BINAP or other suitable ligand, and Cs₂CO₃ or other suitable base; in a suitable solvent such as dioxane at 90° C. A compound of formula C′ may be reacted to afford a compound of formula D under conditions suitable for deprotection of a Boc or Cbz protecting group, e.g. hydrochloric acid or trifluoroacetic acid, in a suitable solvent such as dichloromethane or dioxane at room temperature; or Pd/C and H₂ in a suitable solvent such as methanol at room temperature, respectively. A compound of formula D may be reacted with a compound of formula E to afford a compound of formula F under conditions suitable for a reductive amination reaction such as those stated above. A compound of formula D may be reacted with a compound of formula G to afford a compound of formula H under conditions suitable for an aryl amination reaction, e.g. diisopropylethylamine or triethylamine in a suitable solvent such as dimethylsulfoxide at 80-100° C. As needed, mixtures of enantiomers or diastereomers of the compounds may be resolved into their constituent enantiomers or diastereomers using techniques known to one skilled in the art, including but not limited to preparative high performance liquid chromatography or preparative supercritical fluid chromatography.

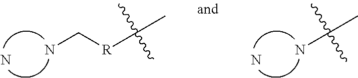

each represent a portion of linker (L) and are a primary or secondary amine, optionally cyclized into a heterocyclic ring. PG is a protecting group such as Boc or Cbz. LG is a leaving group such as a halide or tosylate. All other variables are as defined herein.

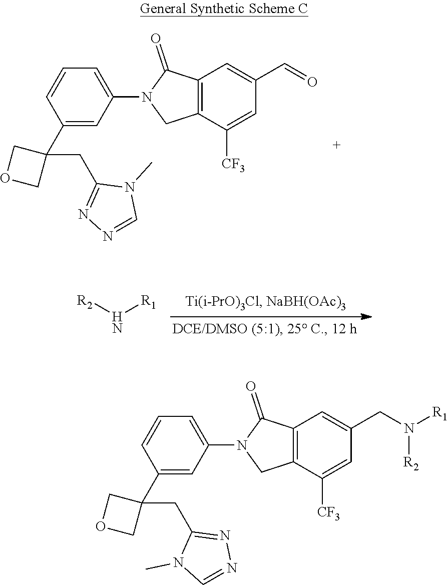

To a solution of 2-[3-[3-[(4-methyl-1,2,4-triazol-3-yl)methyl]oxetan-3-yl]phenyl]-3-oxo-7-(trifluoromethyl)isoindoline-5-carbaldehyde (30 mg, 66 umol) in DCE (2 mL) and DMSO (0.4 mL) was added the amine (66 umol) and chloro(triisopropoxy)titanium (131 umol) at 20° C. Th e mixture was stirred at 20° C. for 11 h. Then sodium triacetoxyborohydride (197 umol) was added d at 20° C., and the mixture was stirred at 20° C. for 1 h. The reaction mixture was purified by pre parative HPLC (Welch Xtimate C18 150×25 mm×5 um, phase A: 0.225% formic acid in water phase B: acetonitrile, B %: 0-50%, 20 min). After lyophilization, product was obtained.

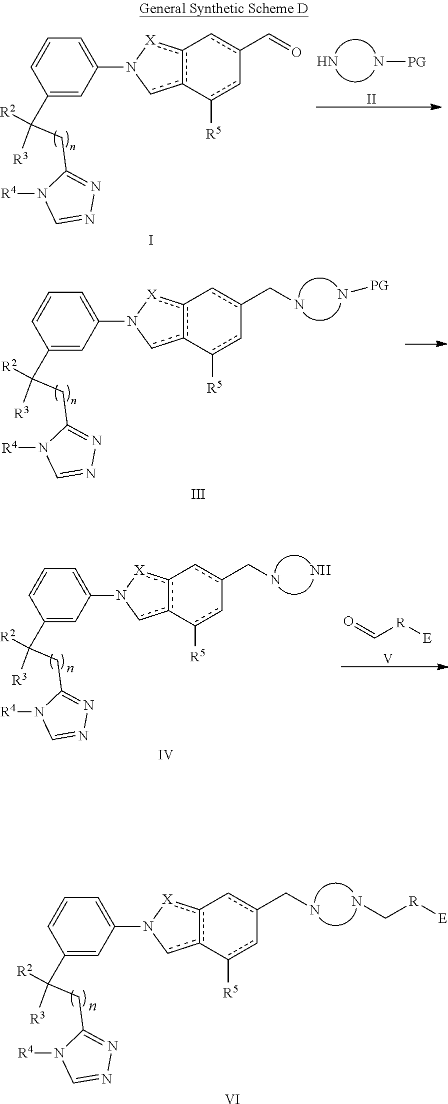

A compound of formula I may be reacted with a compound of formula II to afford a compound of formula III under conditions suitable for a reductive amination reaction, e.g. sodium triacetoxyborohydride and triethylamine or diisopropylethylamine; or sodium cyanoborohydride, sodium acetate, and acetic acid; in a suitable solvent such as isopropanol/dichloromethane, dimethylsulfoxide/dichloromethane, dichloromethane, dichloroethane, or dimethylsulfoxide/dichloroethane at room temperature. A compound of formula III may be reacted to afford a compound of formula IV under conditions suitable for deprotection of a Boc or Cbz protecting group, e.g. hydrochloric acid or trifluoroacetic acid, in a suitable solvent such as dichloromethane or dioxane at room temperature; or Pd/C and H₂ in a suitable solvent such as methanol at room temperature, respectively. A compound of formula IV may be reacted with a compound of formula V to afford a compound of formula VI under conditions suitable for a reductive amination reaction such as those stated above. As needed, mixtures of enantiomers or diastereomers of any compounds II, III, IV, V, VI, VII, or VIII may be resolved into their constituent enantiomers or diastereomers using techniques known to one skilled in the art, including but not limited to preparative high performance liquid chromatography or preparative supercritical fluid chromatography. Herein X represents a heteroatom or optionally substituted C atom; R represents an optional linker or portion of a linker; PG represents a Boc or Cbz protecting group;

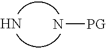

represents a Boc- or Cbz-protected secondary amine, optionally cyclized into a 4 to 8 membered heterocyclic ring, including spirocycles; and E is:

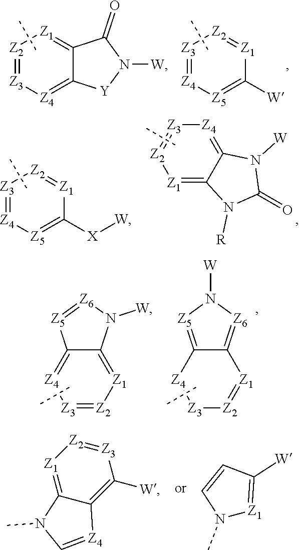

wherein W is:

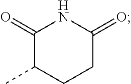

### W′ is:

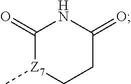

each Z₁, Z₂, Z₃, Z₄, Z₅, Z₆, and Z₇ is independently selected from N or CR; Z′₁ is NR or CHR; X is NR or C(O)NR; Y is CH₂ or C═O; and R is H or an optional substituent.

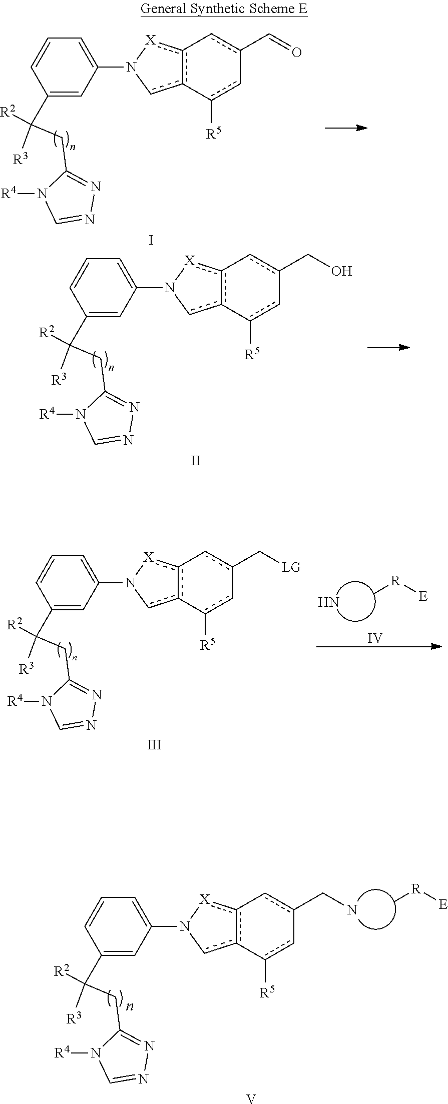

A compound of formula I may be reacted with potassium; trifluoro(vinyl)boranuide to afford a compound of formula II under conditions suitable for a Suzuki cross-coupling reaction, e.g. sodium carbonate, Pd(dppf)Cl₂·CH₂Cl₂, and dioxane/water. A compound of formula II may be converted to a compound of formula III under conditions suitable for an oxidation reaction, such as potassium osmate (VI), NaIO₄, 2,6-lutidine, and dioxane/water. A compound of formula III may be converted to a compound of formula IV under conditions suitable for an aldehyde reduction reaction, such as sodium borohydride and EtOH; sodium(triacetoxy)borohydride and methanol or sodium(triacetoxy)borohydride and dichrolomethane. A compound of formula IV may be converted to a compound of formula V under conditions suitable for a chlorination or mesylation reaction, e.g. thionyl chloride and dichloromethane; N-chlorosuccinimide, triphenylphosphine, sodium bicarbonate, and tetrahydrofuran; or methylsulfonyl methanesulfonate, triethylamine and dicholormethane. A compound of formula V may be reacted with a compound of formula VI to afford a compound of formula VII under conditions suitable for a substitution reaction, e.g. triethylamine or diisopropyl ethylamine and a suitable solvent such as tetrahydrofuran/acetonitrile, dimethylsulfoxide, or dimethyl formamide.

As needed, mixtures of enantiomers or diastereomers of any compounds I, II, III, IV, or V may be resolved into their constituent enantiomers or diastereomers using techniques known to one skilled in the art, including but not limited to preparative high performance liquid chromatography or preparative supercritical fluid chromatography. Herein LG represents a leaving group such as —Cl or —OMs; R represents an optional linker or portion of a linker; X represents a heteroatom or optionally substituted C atom;

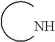

represents a primary or secondary amine, optionally cyclized into a 4 to 8 membered heterocyclic ring; and E is:

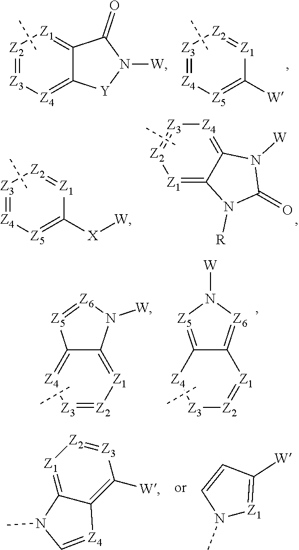

wherein W is:

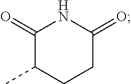

### W′ is:

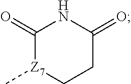

each Z₁, Z₂, Z₃, Z₄, Z₅, Z₆, and Z₇ is independently selected from N or CR; Z′₁ is NR or CHR; X is NR or C(O)NR; Y is CH₂ or C═O; and R is H or an optional substituent.

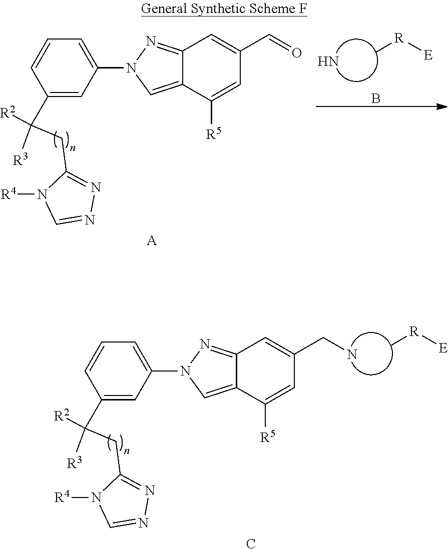

A compound of formula A may be reacted with a compound of formula B to afford a compound of formula C under conditions suitable for a reductive amination reaction, e.g. sodium triacetoxyborohydride and triethylamine or diisopropylethylamine; or sodium cyanoborohydride, sodium acetate, and acetic acid; in a suitable solvent such as isopropanol/dichloromethane, dimethylsulfoxide/dichloromethane, dichloromethane, dichloroethane, or dimethylsulfoxide/dichloroethane at room temperature. As needed, mixtures of enantiomers or diastereomers of the compounds may be resolved into their constituent enantiomers or diastereomers using techniques known to one skilled in the art, including but not limited to preparative high performance liquid chromatography or preparative supercritical fluid chromatography.

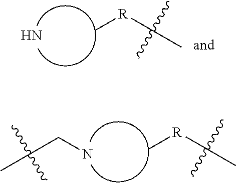

each represent a portion of linker (L) as defined herein where

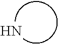

is a primary or secondary amine, optionally cyclized into a heterocyclic ring. All other variables are as defined herein.

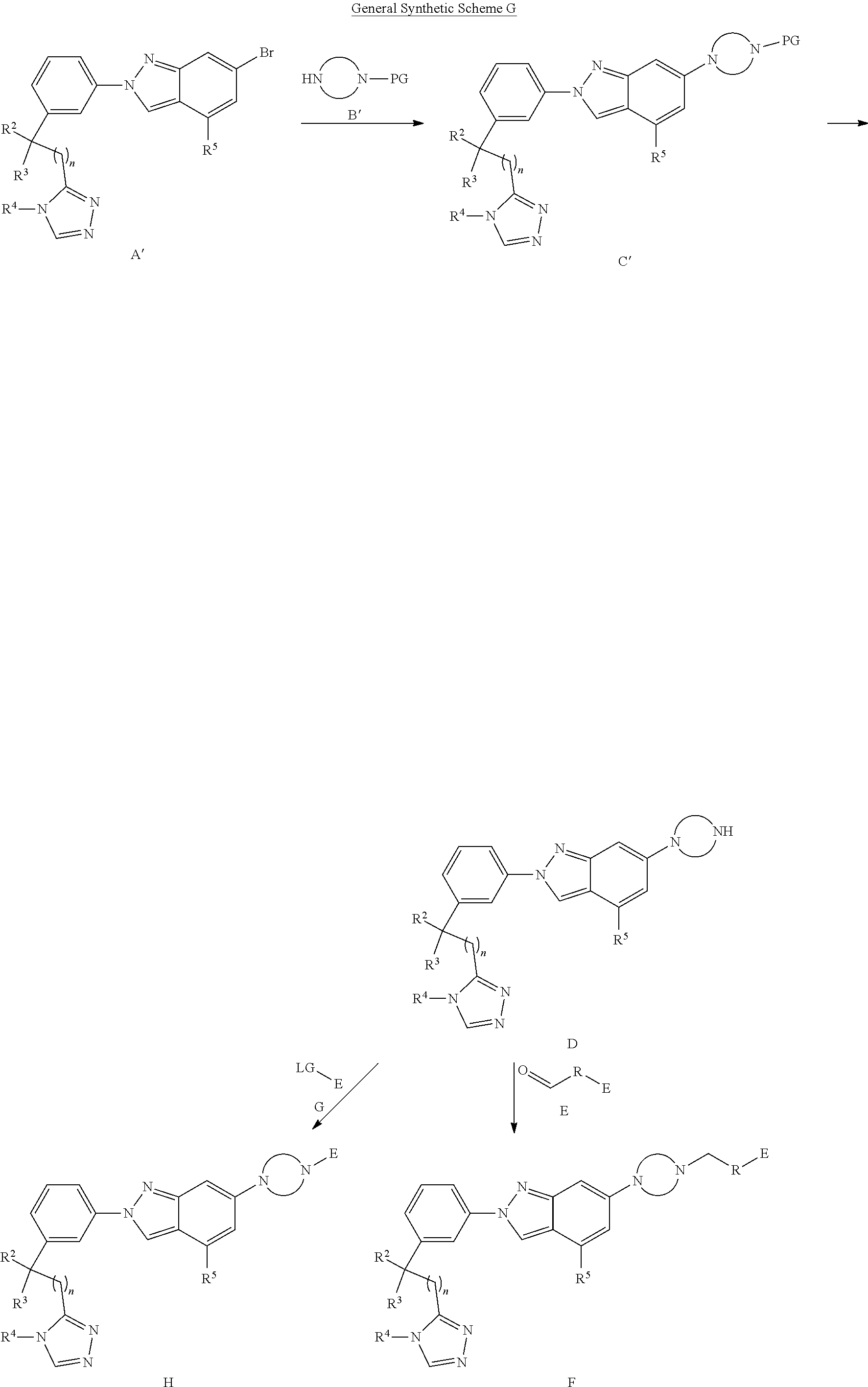

A compound of formula A′ may be reacted with a compound of formula B′ to afford a compound of formula C under conditions suitable for an aryl amination reaction, e.g. Pd(OAc)₂ or other suitable palladium catalyst, BINAP or other suitable ligand, and Cs₂CO₃ or other suitable base; in a suitable solvent such as dioxane at 90° C. A compound of formula C′ may be reacted to afford a compound of formula D under conditions suitable for deprotection of a Boc or Cbz protecting group, e.g. hydrochloric acid or trifluoroacetic acid, in a suitable solvent such as dichloromethane or dioxane at room temperature; or Pd/C and H₂ in a suitable solvent such as methanol at room temperature, respectively. A compound of formula D may be reacted with a compound of formula E to afford a compound of formula F under conditions suitable for a reductive amination reaction such as those stated above. A compound of formula D may be reacted with a compound of formula G to afford a compound of formula H under conditions suitable for an aryl amination reaction, e.g. diisopropylethylamine or triethylamine in a suitable solvent such as dimethylsulfoxide at 80-100° C. As needed, mixtures of enantiomers or diastereomers of the compounds may be resolved into their constituent enantiomers or diastereomers using techniques known to one skilled in the art, including but not limited to preparative high performance liquid chromatography or preparative supercritical fluid chromatography.

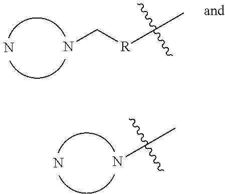

each represent a portion of linker (L) and are a primary or secondary amine, optionally cyclized into a heterocyclic ring. PG is a protecting group such as Boc or Cbz. LG is a leaving group such as a halide or tosylate. All other variables are as defined herein.

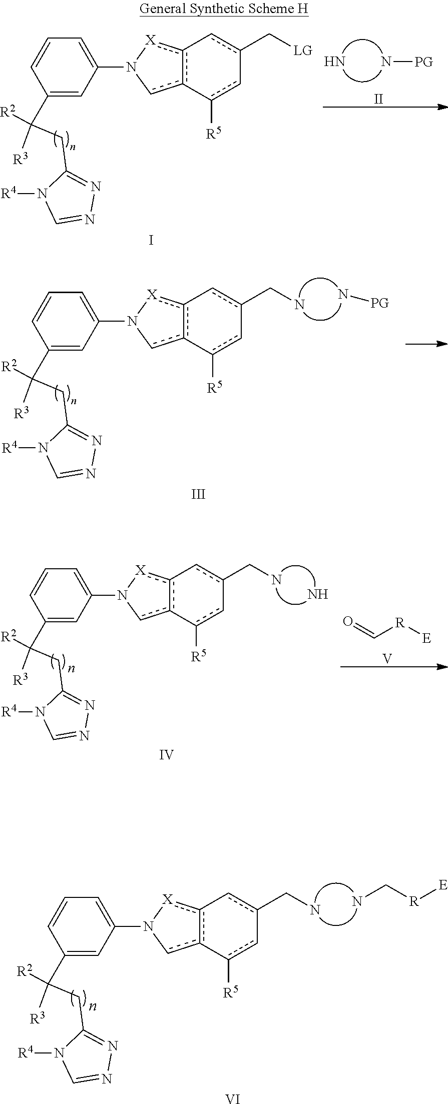

A compound of formula I may be reacted with a compound of formula II to afford a compound of formula III under conditions suitable for a substitution reaction, e.g. triethylamine or diisopropyl ethylamine and a suitable solvent such as tetrahydrofuran/acetonitrile, dimethylsulfoxide, or dimethyl formamide. A compound of formula III may be reacted to afford a compound of formula IV under conditions suitable for deprotection of a Boc or Cbz protecting group, e.g. hydrochloric acid or trifluoroacetic acid, in a suitable solvent such as dichloromethane or dioxane at room temperature; or Pd/C and H₂ in a suitable solvent such as methanol at room temperature, respectively. A compound of formula IV may be reacted with a compound of formula V to afford a compound of formula IV under conditions suitable for a reductive amination reaction such as those stated above.

As needed, mixtures of enantiomers or diastereomers of any compounds I, II, III, IV, or V may be resolved into their constituent enantiomers or diastereomers using techniques known to one skilled in the art, including but not limited to preparative high performance liquid chromatography or preparative supercritical fluid chromatography. Herein LG represents a leaving group such as —Cl or —OMs; R represents an optional linker or portion of a linker; X represents a heteroatom or optionally substituted C atom;

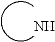

represents a primary or secondary amine, optionally cyclized into a 4 to 8 membered heterocyclic ring; and E is:

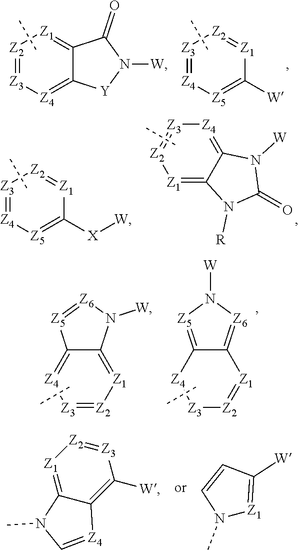

wherein W is:

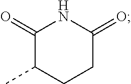

### W′ is:

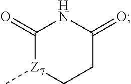

each Z₁, Z₂, Z₃, Z₄, Z₅, Z₆ and Z₇ is independently selected from N or CR; Z′₁ is NR or CHR; X is NR or C(O)NR; Y is CH₂ or C═O; and R is H or an optional substituent.

### Intermediate 1: 3-(3-((4-methyl-4H-1,2,4-triazol-3-yl)methyl)oxetan-3-yl)aniline

### Step 1: Preparation of ethyl 2-(oxetan-3-ylidene)acetate

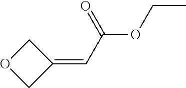

To a mixture of ethyl 2-diethoxyphosphorylacetate (46.67 g, 208 mmol) in tetrahydrofuran (500 mL) was slowly added 1,8-diazabicyclo[5.4.0]undec-7-ene (31.69 g, 208 mmol) at 0° C., and the mixture was stirred for 1 h at 0° C. To the above suspension was added oxetan-3-one (10.0 g, 139 mmol) dropwise. The reaction was stirred at 25° C. for 16 h, then concentrated under reduced pressure. The residue was purified by preparative thin layer chromatography (petroleum ether:ethyl acetate=3:1) to afford ethyl 2-(oxetan-3-ylidene)acetate (10 g, 51%) as a colorless oil.

### Step 2: Preparation of ethyl 2-(3-(3-nitrophenyl)oxetan-3-yl)acetate

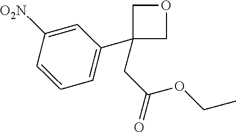 [SMILES: CCOC(=O)CC1(c2cccc([N+](=O)[O-])c2)COC1]

To a solution of aqueous potassium hydroxide (1.5 M, 47.9 mL) in dioxane (36 mL) was added chlororhodium (1Z,5Z)-cycloocta-1,5-diene (1.24 g, 2.5 mmol). The reaction mixture was stirred at 25° C. for 1 h, then (3-nitrophenyl)boronic acid (12 g, 72 mmol) and ethyl 2-(oxetan-3-ylidene)acetate (6.85 g, 48 mmol) in dioxane (24 mL) were added. The reaction was stirred at 25° C. for 11 h. Water (500 mL) was added and the aqueous layer was extracted with ethyl acetate (3×300 mL). The combined organic phase was washed with brine (300 mL), dried over anhydrous sodium sulfate, filtered, and concentrated. The residue was purified by column chromatography (petroleum ether/ethyl acetate=20/1 to 2/1) to afford ethyl 2-(3-(3-nitrophenyl)oxetan-3-yl)acetate (4 g, 21%) as a yellow solid.

### Step 3: Preparation of 2-(3-(3-nitrophenyl)oxetan-3-yl)acetohydrazide

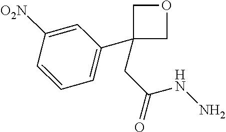 [SMILES: NNC(=O)CC1(c2cccc([N+](=O)[O-])c2)COC1]

To a solution of ethyl 2-[3-(3-nitrophenyl)oxetan-3-yl]acetate (6.0 g, 23 mmol) in ethanol (30 mL) was added hydrazine hydrate (18.49 g, 362 mmol). The mixture was stirred at 80° C. for 12 h. To the reaction mixture was added water (30 mL) and the mixture was extracted with ethyl acetate (3×30 mL). The combined organic phase was washed with brine (20 mL), dried over anhydrous sodium sulfate, filtered, and concentrated to afford 2-(3-(3-nitrophenyl)oxetan-3-yl)acetohydrazide (1.5 g, crude) as a white solid. MS (ESI): m/z 252.0 [M+H]⁺.

### Step 4: Preparation of N-methyl-2-(2-(3-(3-nitrophenyl)oxetan-3-yl)acetyl)hydrazinecarbothioamide

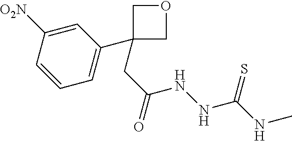 [SMILES: CNC(=S)NNC(=O)CC1(c2cccc([N+](=O)[O-])c2)COC1]

To a solution of 2-[3-(3-nitrophenyl)oxetan-3-yl]acetohydrazide (5.4 g, 21 mmol) in tetrahydrofuran (54 mL) was added methylimino(thioxo)methane (2.36 g, 32 mmol). The mixture was stirred at 50° C. for 1 h, then concentrated in vacuo to afford N-methyl-2-(2-(3-(3-nitrophenyl)oxetan-3-yl)acetyl)hydrazinecarbothioamide (6.8 g, crude) as a white solid. MS (ESI): m/z 325.0 [M+H]⁺.

### Step 5: Preparation of 4-methyl-5-((3-(3-nitrophenyl)oxetan-3-yl)methyl)-4H-1,2,4-triazole-3-thiol

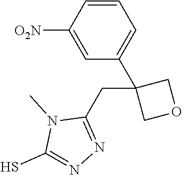 [SMILES: Cn1c(S)nnc1CC1(c2cccc([N+](=O)[O-])c2)COC1]

A solution of 1-methyl-3-[[2-[3-(3-nitrophenyl)oxetan-3-yl]acetyl]amino]thiourea (6.8 g, 21 mmol) in sodium hydroxide (1 M, 210 mL) was stirred at 25° C. for 12 h. The pH was adjusted to 4 by the addition of 4 M aqueous hydrochloric acid and the aqueous layer was extracted with ethyl acetate (3×30 mL). The combined organic layers were dried over anhydrous sodium sulfate, filtered, and concentrated under reduced pressure to afford 4-methyl-5-((3-(3-nitrophenyl)oxetan-3-yl)methyl)-4H-1,2,4-triazole-3-thiol (5.8 g, 90%) as a white solid. MS (ESI): m/z 307.1 [M+H]⁺.

### Step 6: Preparation of 4-methyl-3-((3-(3-nitrophenyl)oxetan-3-yl)methyl)-4H-1,2,4-triazole

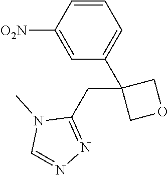 [SMILES: Cn1cnnc1CC1(c2cccc([N+](=O)[O-])c2)COC1]

To a solution of 4-methyl-5-[[3-(3-nitrophenyl)oxetan-3-yl]methyl]-1,2,4-triazole-3-thiol (5.8 g, 19 mmol) in water (30 mL) was added sodium nitrite (13.06 g, 189 mmol) and nitric acid (1 M, 189 mL). The mixture was stirred at 0° C. for 1 h. Water (50 mL) was added, and the mixture was extracted with ethyl acetate (3×50 mL). The combined organic phase was washed with brine (2×30 mL), dried over anhydrous sodium sulfate, filtered and concentrated. The crude product was purified by reversed-phase HPLC ([water (FA)-ACN]; B %: 5%-35%, 21 min) to afford 4-methyl-3-((3-(3-nitrophenyl)oxetan-3-yl)methyl)-4H-1,2,4-triazole (2.9 g, 56%) as a white solid. MS (ESI): m/z 275.0 [M+H]⁺.

### Step 7: Preparation of 3-(3-((4-methyl-4H-1,2,4-triazol-3-yl)methyl)oxetan-3-yl)aniline

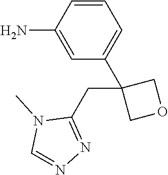 [SMILES: Cn1cnnc1CC1(c2cccc(N)c2)COC1]

To a solution of 4-methyl-3-[[3-(3-nitrophenyl)oxetan-3-yl]methyl]-1,2,4-triazole (2.9 g, 11 mmol) in trifluoroethanol (30 mL) was added 10% palladium on carbon (600 mg) under nitrogen atmosphere. The suspension was degassed under vacuum and purged with hydrogen several times. The mixture was stirred under hydrogen (15 psi) at 25° C. for 1 h. The reaction was filtered, and the filtrate was concentrated to afford 3-(3-((4-methyl-4H-1,2,4-triazol-3-yl)methyl)oxetan-3-yl)aniline (2.5 g, 97%) as a white solid. MS (ESI): m/z 244.8 [M+H]⁺.

### Intermediate 2: 2-(3-(3-((4-methyl-4H-1,2,4-triazol-3-yl)methyl)oxetan-3-yl)phenyl)-3-oxo-7-(trifluoromethyl)isoindoline-5-carbaldehyde

### Step 1: Preparation of methyl 2-methyl-3-(trifluoromethyl)benzoate

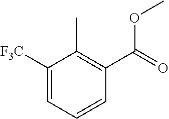

To a solution of 2-methyl-3-(trifluoromethyl)benzoic acid (20 g, 98 mmol) in methanol (200 mL) was added sulfuric acid (9.61 g, 98 mmol). The mixture was stirred at 80° C. for 2 h, then concentrated under reduced pressure to remove methanol, followed by the addition of saturated aqueous sodium bicarbonate to bring the pH to 7-8, and extracted with ethyl acetate (3×60 mL). The combined organic layers were washed with brine (2×50 mL), dried over anhydrous sodium sulfate, filtered, and concentrated under reduced pressure to afford methyl 2-methyl-3-(trifluoromethyl)benzoate (19 g, 89%) as a colorless oil.

### Step 2: Preparation of methyl 5-bromo-2-methyl-3-(trifluoromethyl)benzoate

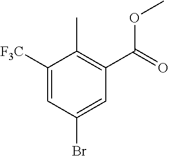 [SMILES: COC(=O)c1cc(Br)cc(C(F)(F)F)c1C]

To a solution of methyl 2-methyl-3-(trifluoromethyl)benzoate (10 g, 46 mmol) in acetic acid (100 mL) was added nitric acid (58.0 g, 552 mmol, 60%) and bromine (8.79 g, 55 mmol). Then silver nitrate (2.5 M, 32.3 mL) in water was added to the mixture over 30 min. The reaction mixture was quenched by the addition of water (50 mL), diluted with water (50 mL), and extracted with ethyl acetate (3×60 mL). The combined organic layers were washed with brine (3×60 mL), dried over anhydrous sodium sulfate, filtered, and concentrated under reduced pressure. The residue was purified by flash silica gel chromatography (0-0.2% ethyl acetate/petroleum ether) to afford methyl 5-bromo-2-methyl-3-(trifluoromethyl)benzoate (10 g, 73%) as a colorless oil.

### Step 3: Preparation of 3-(methoxycarbonyl)-4-methyl-5-(trifluoromethyl)benzoic acid

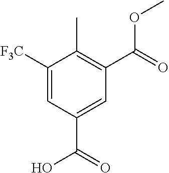 [SMILES: COC(=O)c1cc(C(=O)O)cc(C(F)(F)F)c1C]

To a solution of methyl 5-bromo-2-methyl-3-(trifluoromethyl)benzoate (10 g, 34 mmol), oxalic acid (4.55 g, 51 mmol), acetic anhydride (5.15 g, 51 mmol) and diisopropylethylamine (6.53 g, 51 mmol) in N,N-dimethylformamide (100 mL) was added palladium(II) acetate (755.8 mg, 3.4 mmol) and 4,5-bis(diphenylphosphino)-9,9-dimethyl-9H-xanthene (973.9 mg, 1.7 mmol). The reaction mixture was stirred at 100° C. for 12 h under nitrogen atmosphere. The reaction mixture was quenched by the addition of 1M hydrochloric acid (80 mL) to pH 1-2, then diluted with water (30 mL) and extracted with ethyl acetate (3×70 mL). The combined organic layers were washed with brine (3×60 mL), dried over anhydrous sodium sulfate, filtered, and concentrated. The residue was purified by flash silica gel chromatography (0-20% ethyl acetate/petroleum ether) to afford 3-(methoxycarbonyl)-4-methyl-5-(trifluoromethyl)benzoic acid (5.2 g, 59%) as a yellow solid.

### Step 4: Preparation of 4-(bromomethyl)-3-(methoxycarbonyl)-5-(trifluoromethyl)benzoic acid

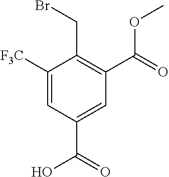 [SMILES: COC(=O)c1cc(C(=O)O)cc(C(F)(F)F)c1CBr]

To a solution of 3-methoxycarbonyl-4-methyl-5-(trifluoromethyl)benzoic acid (8.0 g, 30 mmol) and N-bromosuccinimide (8.15 g, 46 mmol) in carbon tetrachloride (160 mL) was added benzoyl peroxide (2.22 g, 9.2 mmol). The mixture was stirred at 80° C. for 16 h, then concentrated under reduced pressure. The residue was purified by flash silica gel chromatography (0-20% ethyl acetate/petroleum ether) to afford 4-(bromomethyl)-3-methoxycarbonyl-5-(trifluoromethyl)benzoic acid (10.2 g, crude) as a white solid.

### Step 5: Preparation of methyl 2-(bromomethyl)-5-(hydroxymethyl)-3-(trifluoromethyl)benzoate

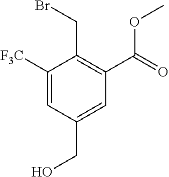 [SMILES: COC(=O)c1cc(CO)cc(C(F)(F)F)c1CBr]

To a solution of 4-(bromomethyl)-3-methoxycarbonyl-5-(trifluoromethyl)benzoic acid (7.0 g, 20 mmol) in tetrahydrofuran (70 mL) was added borane dimethyl sulfide (10 M, 4.1 mL) at 0° C. The mixture was stirred at 15° C. for 12 h, then quenched by methanol (30 mL) at 0° C., stirred at 25° C. for 1 h, and concentrated under reduced pressure. The residue was purified by flash silica gel chromatography (0-20% ethyl acetate/petroleum ether) to afford methyl 2-(bromomethyl)-5-(hydroxymethyl)-3-(trifluoromethyl)benzoate (5.8 g, 86%) as a colorless oil.

### Step 6: Preparation of methyl 2-(bromomethyl)-5-formyl-3-(trifluoromethyl)benzoate

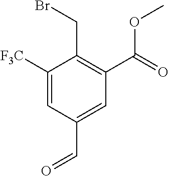 [SMILES: COC(=O)c1cc(C=O)cc(C(F)(F)F)c1CBr]

To a solution of methyl 2-(bromomethyl)-5-(hydroxymethyl)-3-(trifluoromethyl)benzoate (5.8 g, 18 mmol) in ethyl acetate (60 mL) was added 2-iodylbenzoic acid (7.45 g, 27 mmol). The reaction mixture was stirred at 70° C. for 6 h. The reaction mixture was filtered and concentrated under reduced pressure. The residue was purified by flash silica gel chromatography (0-20% ethyl acetate/petroleum ether) to afford methyl 2-(bromomethyl)-5-formyl-3-(trifluoromethyl)benzoate (5.1 g, 88%) as a colorless oil.

### Step 7: Preparation of 2-(3-(3-((4-methyl-4H-1,2,4-triazol-3-yl)methyl)oxetan-3-yl)phenyl)-3-oxo-7-(trifluoromethyl)isoindoline-5-carbaldehyde

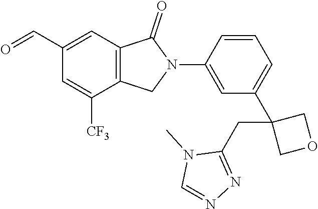 [SMILES: Cn1cnnc1CC1(c2cccc(N3Cc4c(cc(C=O)cc4C(F)(F)F)C3=O)c2)COC1]

A solution of 3-[3-[(4-methyl-1,2,4-triazol-3-yl)methyl]oxetan-3-yl]aniline (1.13 g, 4.6 mmol) and methyl 2-(bromomethyl)-5-formyl-3-(trifluoromethyl)benzoate (1.5 g, 4.6 mmol) in acetonitrile (41 mL) and water (20.5 mL) was cooled to 0° C. before the addition of silver nitrate (1.02 g, 6 mmol) in water (5.8 mL). The reaction was stirred for 12 h at 25° C., followed by the addition of solid sodium bicarbonate until the pH of the solution was 8. The mixture was filtered through Celite, and washed with acetonitrile (10 mL), then with dichloromethane/ethyl acetate (50 mL, 9:1). The organic layer was separated and dried over sodium sulfate, filtered, and concentrated. The residue was purified by preparative thin layer chromatography (dichloromethane:methanol=15:1) to afford 2-(3-(3-((4-methyl-4H-1,2,4-triazol-3-yl)methyl)oxetan-3-yl)phenyl)-3-oxo-7-(trifluoromethyl)isoindoline-5-carbaldehyde (740 mg, 35%) as a yellow solid.

### Intermediate 3: (1r, 4r)-4-((1-(2-(2,6-dioxopiperidin-3-yl)-4-fluoro-1-oxoisoindolin-5-yl)piperidin-4-yl)oxy)cyclohexanecarbaldehyde

### Step 1: Preparation of benzyl 4-(4-ethoxycarbonylcyclohexoxy)piperidine-1-carboxylate

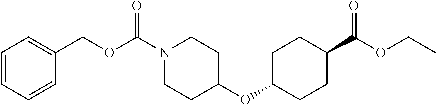 [SMILES: CCOC(=O)[C@H]1CC[C@H](OC2CCN(C(=O)OCc3ccccc3)CC2)CC1]

To a solution of ethyl 4-hydroxycyclohexanecarboxylate (70.0 g, 406 mmol) in tetrahydrofuran (1400 mL) was added chlorotrimethylsilane (48.57 g, 447 mmol) and triethylamine (61.69 g, 610 mmol) at 0° C. The mixture was stirred at 25° C. for 0.5 h, then filtered and the filtrate was concentrated under reduced pressure. To the above residue was added dichloromethane (1600 mL) and benzyl 4-oxopiperidine-1-carboxylate (123.25 g, 528 mmol). The mixture was cooled to −60° C., and triethylsilane (94.53 g, 813 mmol) and trimethylsilyl trifluoromethanesulfonate (81.30 g, 366 mmol) were added dropwise, then the mixture was stirred at 25° C. for 7.5 h, diluted with water (1500 mL), and extracted with ethyl acetate (2×1000 mL). The combined organic layers were dried over anhydrous sodium sulfate, filtered, and concentrated. The residue was purified by column chromatography (petroleum ether:ethyl acetate=1:0 to 5:1) to afford benzyl 4-(4-ethoxycarbonylcyclohexoxy)piperidine-1-carboxylate (120 g, 76%) as a colorless oil. MS (ESI): m/z 350.8 [M+H]⁺.

### Step 2: Preparation of benzyl 4-(4-formylcyclohexoxy)piperidine-1-carboxylate

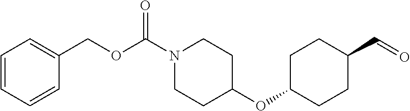 [SMILES: O=C[C@H]1CC[C@H](OC2CCN(C(=O)OCc3ccccc3)CC2)CC1]

To a solution of benzyl 4-(4-ethoxycarbonylcyclohexoxy)piperidine-1-carboxylate (115 g, 295 mmol) in dichloromethane (1500 mL) was added diisobutylaluminum hydride (1 M, 443 mL) at −70° C. The mixture was stirred for 2 h, then 2 M hydrochloric acid (100 mL) was slowly added at −70° C., then sodium sulfate was added. The mixture was filtered and concentrated under vacuum. The residue was purified by flash silica gel chromatography (petroleum ether:ethyl acetate=1:0 to 1:1) to afford benzyl 4-(4-formylcyclohexoxy)piperidine-1-carboxylate (168 g, 82%) as a yellow oil.

### Step 3: Preparation of benzyl 4-[4-(dimethoxymethyl)cyclohexoxy]piperidine-1-carboxylate

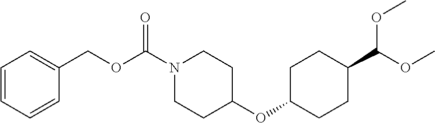 [SMILES: COC(OC)[C@H]1CC[C@H](OC2CCN(C(=O)OCc3ccccc3)CC2)CC1]

To a solution of benzyl 4-(4-formylcyclohexoxy)piperidine-1-carboxylate (120 g, 347 mmol) in methanol (1000 mL) was added trimethoxymethane (73.73 g, 695 mmol) and p-toluenesulfonic acid monohydrate (2.0 g, 11 mmol). The mixture was stirred at 25° C. for 1 h, then concentrated. Saturated sodium bicarbonate solution (200 mL) was added, and the mixture was extracted with ethyl acetate (3×100 mL). The organic phase was washed with brine (100 mL), dried over anhydrous sodium sulfate, filtered, and concentrated. The residue was purified by flash silica gel chromatography (petroleum ether:ethyl acetate=1:0 to 4:1) to afford benzyl 4-[4-(dimethoxymethyl)cyclohexoxy]piperidine-1-carboxylate (240 g, 88%) as a yellow oil.

### Step 4: Preparation of 4-[4-(dimethoxymethyl)cyclohexoxy]piperidine

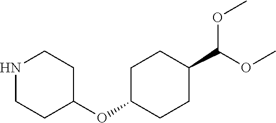 [SMILES: COC(OC)[C@H]1CC[C@H](OC2CCNCC2)CC1]

To a solution of benzyl 4-[4-(dimethoxymethyl)cyclohexoxy]piperidine-1-carboxylate (120 g, 306 mmol) in methanol (900 mL) was added 10% palladium on carbon (10 g) under nitrogen. The suspension was degassed under vacuum and purged with hydrogen several times. The mixture was stirred under hydrogen (50 psi) at 50° C. for 12 h, then filtered and concentrated under vacuum to afford 4-[4-(dimethoxymethyl)cyclohexoxy]piperidine (78.5 g, 99%) as a yellow oil.

### Step 5: Preparation of methyl 2-bromo-3,4-difluoro-benzoate

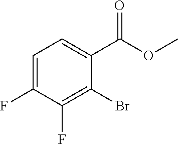

To a solution of 2-bromo-3,4-difluoro-benzoic acid (123 g, 519 mmol) in methanol (1300 mL) was added thionyl chloride (123.49 g, 1 mol) at 0° C. The mixture was stirred at 70° C. for 12 h, then concentrated under vacuum to give methyl 2-bromo-3,4-difluoro-benzoate (251.2 g, 96%) as a yellow oil.

### Step 6: Preparation of methyl 2-bromo-4-[4-[4-(dimethoxymethyl)cyclohexoxy]-1-piperidyl]-3-fluoro-benzoate

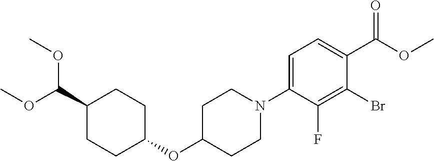

To a solution of 4-[4-(dimethoxymethyl)cyclohexoxy]piperidine (78.0 g, 303 mmol) in dimethylsulfoxide (1000 mL) was added N,N-diisopropylethylamine (111.91 g, 866 mmol) and methyl 2-bromo-3,4-difluoro-benzoate (72.45 g, 289 mmol). The mixture was stirred at 100° C. for 12 h, then poured into ice-water (1000 mL). The aqueous phase was extracted with ethyl acetate (3×200 mL). The combined organic phase was washed with brine (200 mL), dried over anhydrous sodium sulfate, filtered, and concentrated under vacuum. The residue was purified by silica gel chromatography (petroleum ether:ethyl acetate=7:1 to 1:1) to afford methyl 2-bromo-4-[4-[4-(dimethoxymethyl)cyclohexoxy]-1-piperidyl]-3-fluoro-benzoate (130 g, 92%) as a white solid.

### Step 7: Preparation of methyl 4-(4-(((1r, 4r)-4-(dimethoxymethyl)cyclohexyl)oxy)piperidin-1-yl)-3-fluoro-2-vinylbenzoate

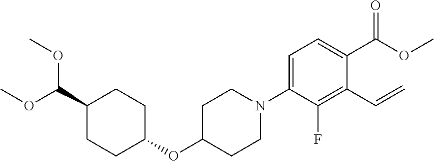

To a stirred solution of methyl 2-bromo-4-[4-[4-(dimethoxymethyl)cyclohexoxy]-1-piperidyl]-3-fluoro-benzoate (125 g, 256 mmol) in 1,4-dioxane (1050 mL) and water (210 mL) was added potassium trifluoro(vinyl)boranuide (102.85 g, 768 mmol), [1,1′-bis(diphenylphosphino)ferrocene]dichloropalladium(II) (14.63 g, 18 mmol) and sodium carbonate (67.82 g, 640 mmol). The reaction mixture was stirred at 110° C. for 12 h, then filtered. The filtrate was diluted with water (800 mL) and extracted with ethyl acetate (2×400 mL). The combined organic layer was washed with brine (2×500 mL), dried over anhydrous sodium sulfate, filtered, and concentrated under vacuum. The residue was purified by column chromatography (petroleum ether:ethyl acetate=1:0 to 7:3) to afford methyl 4-[4-[4-(dimethoxymethyl)cyclohexoxy]-1-piperidyl]-3-fluoro-2-vinyl-benzoate (80 g, 71%) as a yellow oil.

### Step 8: Preparation of methyl 4-(4-(((1r, 4r)-4-(dimethoxymethyl)cyclohexyl)oxy)piperidin-1-yl)-3-fluoro-2-formylbenzoate

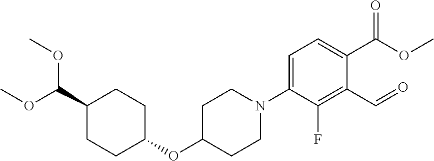

To a stirred solution of methyl 4-[4-[4-(dimethoxymethyl)cyclohexoxy]-1-piperidyl]-3-fluoro-2-vinyl-benzoate (85.0 g, 195 mmol) in 1,4-dioxane (1100 mL) and water (380 mL) was added 2,6-lutidine (45.46 mL, 390 mmol), potassium osmate (VI) (1.44 g, 3.9 mmol) and sodium periodate (43.26 mL, 781 mmol) in portions. The reaction mixture was stirred at 20° C. for 1 h. Water (800 mL) was added and the mixture was filtered. The filtrate was extracted with ethyl acetate (3×600 mL). The combined organic layer was dried over anhydrous sodium sulfate, filtered, and the filtrate was concentrated under vacuum. The residue was purified by column chromatography (petroleum ether:ethyl acetate=1:0 to 7:3) to obtain methyl 4-[4-[4-(dimethoxymethyl)cyclohexoxy]-1-piperidyl]-3-fluoro-2-formyl-benzoate (65 g, 76%) as a yellow oil.

### Step 9: Preparation of 3-(5-(4-(((1r, 4r)-4-(dimethoxymethyl)cyclohexyl)oxy)piperidin-1-yl)-4-fluoro-1-oxoisoindolin-2-yl)piperidine-2,6-dione

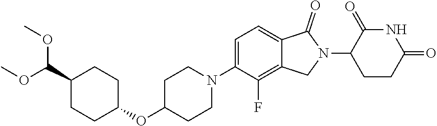 [SMILES: COC(OC)[C@H]1CC[C@H](OC2CCN(c3ccc4c(c3F)CN(C3CCC(=O)NC3=O)C4=O)CC2)CC1]

To a stirred solution of 3-aminopiperidine-2,6-dione hydrochloride (36.12 g, 219 mmol) in methanol (600 mL) was added sodium acetate (45.0 g, 549 mmol). The mixture was stirred at 25° C. for 30 min. Then methyl 4-[4-[4-(dimethoxymethyl)cyclohexoxy]-1-piperidyl]-3-fluoro-2-formyl-benzoate (80.0 g, 183 mmol) was added. The mixture was stirred at 25° C. for another 0.5 h. Finally, sodium cyanoborohydride (22.98 g, 366 mmol) was added. The resulting mixture was stirred at 40° C. for 15 h. The mixture was filtered and washed with methanol (3×50 mL), then the solid was suspended in water (3 L) and the mixture was stirred at 20° C. for 10 min, filtered, washed with acetonitrile (3×200 mL), then dried to afford 3-[5-[4-[4-(dimethoxymethyl)cyclohexoxy]-1-piperidyl]-4-fluoro-1-oxo-isoindolin-2-yl]piperidine-2,6-dione (76 g, 80%) as a light purple solid. MS (ESI): m/z 518.0 [M+H]⁺.

### Step 10: Preparation of (1r, 4r)-4-((1-(2-(2,6-dioxopiperidin-3-yl)-4-fluoro-1-oxoisoindolin-5-yl)piperidin-4-yl)oxy)cyclohexanecarbaldehyde

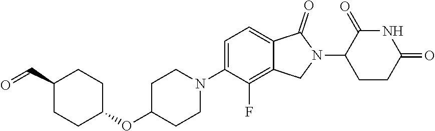 [SMILES: O=C[C@H]1CC[C@H](OC2CCN(c3ccc4c(c3F)CN(C3CCC(=O)NC3=O)C4=O)CC2)CC1]

To a stirred solution of 3-[5-[4-[4-(dimethoxymethyl)cyclohexoxy]-1-piperidyl]-4-fluoro-1-oxo-isoindolin-2-yl]piperidine-2,6-dione (71.0 g, 137 mmol) in acetone (800 mL) and water (80 mL) was added p-toluenesulfonic acid (4.72 g, 27 mmol). The reaction mixture was stirred at 70° C. for 12 h. The mixture was filtered, the filter cake was washed with acetone (3×30 mL), then dried to afford 4-[[1-[2-(2,6-dioxo-3-piperidyl)-4-fluoro-1-oxo-isoindolin-5-yl]-4-piperidyl]oxy]cyclohexanecarbaldehyde (58 g, 89%) as a white solid. MS (ESI): m/z 471.9 [M+H]⁺.

### Intermediate 4: 6-(2,7-diazaspiro[3.5]nonan-2-yl)-2-[3-[3-[(4-methyl-1,2,4-triazol-3-yl)methyl]oxetan-3-yl]phenyl]-4-(trifluoromethyl)isoindolin-1-one

### Step 1: Preparation of methyl 2-methyl-3-(trifluoromethyl)benzoate

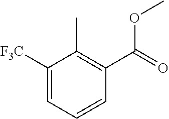

To a solution of 2-methyl-3-(trifluoromethyl)benzoic acid (20 g, 98 mmol) in methanol (200 mL) was added sulfuric acid (5.22 mL, 98 mmol). The mixture was stirred at 80° C. for 2 h, then concentrated. Saturated sodium bicarbonate solution was added to the residue to adjust the pH to 7-8, and the mixture was extracted with ethyl acetate (3×60 mL). The combined organic layers were washed with brine (2×50 mL), dried over anhydrous sodium sulfate, filtered, and concentrated under reduced pressure to afford methyl 2-methyl-3-(trifluoromethyl)benzoate (19 g, 89%) as a colorless oil. ¹H NMR (400 MHz, CDCl₃) δ 7.92 (d, J=8.0 Hz, 1H), 7.77 (d, J=8.0 Hz, 1H), 7.34 (t, J=8.0 Hz, 1H), 3.93 (s, 3H), 2.65 (d, J=1.2 Hz, 3H).

### Step 2: Preparation of methyl 5-bromo-2-methyl-3-(trifluoromethyl)benzoate

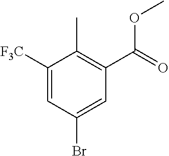 [SMILES: COC(=O)c1cc(Br)cc(C(F)(F)F)c1C]

To a solution of methyl 2-methyl-3-(trifluoromethyl)benzoate (10 g, 46 mmol) in acetic acid (100 mL) was added 60% nitric acid solution (41.4 mL, 552 mmol) and bromine (2.84 mL, 55 mmol). Then 2.5 M silver nitrate solution (32.3 mL) in water was added to the mixture over 30 min. The reaction mixture was quenched by the addition of water (50 mL) and extracted with ethyl acetate (3×60 mL). The combined organic layers were washed with brine (3×60 mL), dried over anhydrous sodium sulfate, filtered, and concentrated under reduced pressure. The residue was purified by flash silica gel chromatography (0-0.2% ethyl acetate/petroleum ether) to afford methyl 5-bromo-2-methyl-3-(trifluoromethyl)benzoate (10 g, 73%) as a colorless oil. ¹H NMR (400 MHz, CDCl₃) δ 8.07 (d, J=1.2 Hz, 1H), 7.90 (s, 1H), 3.94 (s, 3H), 2.60 (s, 3H).

### Step 3: Preparation of methyl 5-bromo-2-(bromomethyl)-3-(trifluoromethyl)benzoate

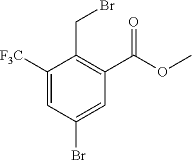 [SMILES: COC(=O)c1cc(Br)cc(C(F)(F)F)c1CBr]

To a solution of methyl 5-bromo-2-methyl-3-(trifluoromethyl)benzoate (5.00 g, 17 mmol) and 1-bromopyrrolidine-2,5-dione (4.49 g, 25 mmol) in carbon tetrachloride (100 mL) was added perbenzoic acid (1.22 g, 5 mmol). The mixture was stirred at 80° C. for 16 h, then concentrated under reduced pressure. Water (30 mL) was added to the residue and extracted with dichloromethane (3×30 mL). The combined organic layers were washed with brine (30 mL), dried over anhydrous sodium sulfate, filtered, and concentrated. The residue was purified by column chromatography on silica gel (petroleum ether/ethyl acetate=30/1 to 5/1) to afford methyl 5-bromo-2-(bromomethyl)-3-(trifluoromethyl)benzoate (6.10 g, 96%) as a yellow oil. ¹H NMR (400 MHz, CDCl₃) δ 8.11 (d, J=2.0 Hz, 1H), 7.87 (d, J=2.0 Hz, 1H), 4.97 (s, 2H), 3.92 (s, 3H).

### Step 4: Preparation of 6-bromo-2-[3-[3-[(4-methyl-1,2,4-triazol-3-yl)methyl]oxetan-3-yl]phenyl]-4-(trifluoromethyl)isoindolin-1-one

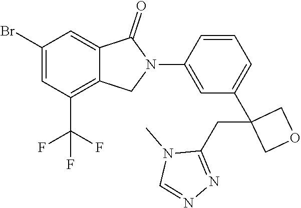 [SMILES: Cn1cnnc1CC1(c2cccc(N3Cc4c(cc(Br)cc4C(F)(F)F)C3=O)c2)COC1]

A solution of methyl 5-bromo-2-(bromomethyl)-3-(trifluoromethyl)benzoate (2.30 g, 6.1 mmol) and 3-[3-[(4-methyl-1,2,4-triazol-3-yl)methyl]oxetan-3-yl]aniline (1.49 g, 6.1 mmol) in acetonitrile (60 mL) and water (30 mL) was cooled to 0° C. before the addition of silver nitrate (1.35 g, 7.9 mmol) in water (7.5 mL). The reaction was stirred at 25° C. for 12 h. Water (200 mL) was added, and the mixture was extracted with ethyl acetate (3×200 mL). The combined organic layers were dried over anhydrous sodium sulfate, filtered, and concentrated. The residue was purified by column chromatography (dichloromethane:methanol=50:1 to 10:1) to afford 6-bromo-2-[3-[3-[(4-methyl-1,2,4-triazol-3-yl)methyl]oxetan-3-yl]phenyl]-4-(trifluoromethyl)isoindolin-1-one (870 mg, 29%) as a yellow solid. ¹H NMR (400 MHz, CDCl₃) δ 8.22 (s, 1H), 8.00 (s, 1H), 7.88 (s, 1H), 7.51-7.60 (m, 1H), 7.45 (t, J=2.0 Hz, 1H), 7.35 (t, J=8.0 Hz, 1H), 6.69 (d, J=8.0 Hz, 1H), 5.16 (d, J=6.4 Hz, 2H), 5.09 (d, J=6.4 Hz, 2H), 4.91 (s, 2H), 3.60 (s, 2H), 2.91 (s, 3H).

### Step 5: Preparation of tert-butyl 2-[2-[3-[3-[(4-methyl-1,2,4-triazol-3-yl)methyl]oxetan-3-yl]phenyl]-3-oxo-7-(trifluoromethyl)isoindolin-5-yl]-2,7-diazaspiro[3.5]nonane-7-carboxylate

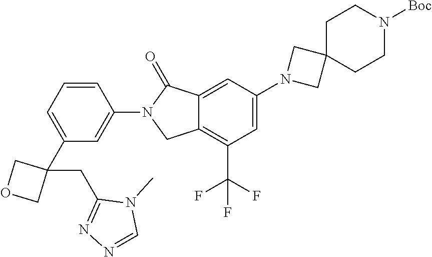 [SMILES: Cn1cnnc1CC1(c2cccc(N3Cc4c(cc(N5CC6(CCN(C(=O)OC(C)(C)C)CC6)C5)cc4C(F)(F)F)C3=O)c2)COC1]

To a solution of 6-bromo-2-[3-[3-[(4-methyl-1,2,4-triazol-3-yl)methyl]oxetan-3-yl]phenyl]-4-(trifluoromethyl)isoindolin-1-one (300 mg, 0.6 mmol) in dioxane (8 mL) was added XPhos Pd G4 (101 mg, 0.1 mmol), cesium carbonate (578 mg, 1.8 mmol) and tert-butyl 2,7-diazaspiro[3.5]nonane-7-carboxylate (160 mg, 0.7 mmol). The mixture was stirred at 90° C. for 10 h, then filtered and concentrated under reduced pressure. The residue was purified by preparative thin layer chromatography (dichloromethane:methanol=10:1) to get tert-butyl 2-[2-[3-[3-[(4-methyl-1,2,4-triazol-3-yl)methyl]oxetan-3-yl]phenyl]-3-oxo-7-(trifluoromethyl)isoindolin-5-yl]-2,7-diazaspiro[3.5]nonane-7-carboxylate (240 mg, 62%) as a yellow solid. ¹H NMR (400 MHz, CDCl₃) δ 7.42-7.51 (m, 2H), 7.31-7.37 (m, 1H), 7.02 (d, J=1.6 Hz, 1H), 6.82 (d, J=1.6 Hz, 1H), 6.63 (d, J=7.2 Hz, 1H), 5.10-5.16 (m, 2H), 5.04-5.09 (m, 2H), 4.84 (s, 2H), 3.75 (s, 4H), 3.64 (s, 2H), 3.40-3.48 (m, 5H), 2.96 (s, 3H), 1.79-1.84 (m, 4H), 1.48 (s, 9H); MS (ESI) m/z: 653.5 [M+H]⁺.

### Step 6: Preparation of 6-(2,7-diazaspiro[3.5]nonan-2-yl)-2-[3-[3-[(4-methyl-1,2,4-triazol-3-yl)methyl]oxetan-3-yl]phenyl]-4-(trifluoromethyl)isoindolin-1-one

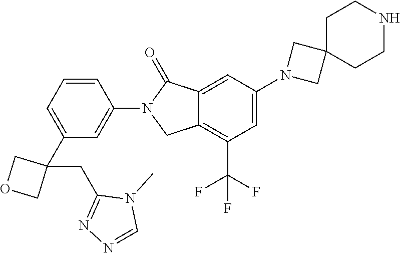 [SMILES: Cn1cnnc1CC1(c2cccc(N3Cc4c(cc(N5CC6(CCNCC6)C5)cc4C(F)(F)F)C3=O)c2)COC1]

To a solution of tert-butyl 2-[2-[3-[3-[(4-methyl-1,2,4-triazol-3-yl)methyl]oxetan-3-yl]phenyl]-3-oxo-7-(trifluoromethyl)isoindolin-5-yl]-2,7-diazaspiro[3.5]nonane-7-carboxylate (240 mg, 0.4 mmol) in dichloromethane (3 mL) was added trifluoroacetic acid (1.54 g, 14 mmol). The mixture was stirred at 25° C. for 0.5 h, then concentrated under reduced pressure to afford 6-(2,7-diazaspiro[3.5]nonan-2-yl)-2-[3-[3-[(4-methyl-1,2,4-triazol-3-yl)methyl]oxetan-3-yl]phenyl]-4-(trifluoromethyl)isoindolin-1-one trifluroacetate (200 mg, 98%) as a yellow oil. MS (ESI) m/z: 553.5 [M+H]⁺.

### Intermediate 5: 3-[(4-methyl-1,2,4-triazol-3-yl)methyl]-3-[3-[1-oxo-6-(1-piperidylmethyl)-4-(trifluoromethyl)isoindolin-2-yl]phenyl]cyclobutanecarbonitrile

### Step 1: Preparation of 3-oxocyclobutanecarbonitrile

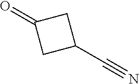

To a solution of 3-methylenecyclobutanecarbonitrile (30.00 g, 322 mmol) and ruthenium(iii)chloride hydrate (1.50 g, 6.7 mmol) in acetonitrile (645 mL), dichloromethane (645 mL) and water (945 mL) was added sodium periodate (73.6 mL, 1.3 mol) portion wise at 0° C. Then the mixture was stirred for 10 h at 25° C., diluted with dichloromethane (1000 mL) and washed with water (1000 mL). The organic layer was washed with brine (1000 mL), dried over anhydrous sodium sulfate, filtered, and concentrated. The residue was purified by column chromatography (petroleum ether:ethyl acetate=10:1 to 5:1) to give 3-oxocyclobutanecarbonitrile (28 g, 91%) as a light yellow solid. ¹H NMR (400 MHz, CDCl₃) δ 3.49 (d, J=8.1 Hz, 4H), 3.27-3.16 (m, 1H).

### Step 2: Preparation of ethyl 2-(3-cyanocyclobutylidene)acetate

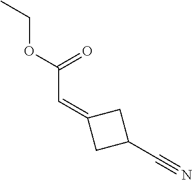

To a solution of 3-oxocyclobutanecarbonitrile (20.00 g, 210 mmol) in dichloromethane (300 mL) was added ethyl (triphenylphosphoranylidene)acetate (75.00 g, 215 mmol) at 0° C. The mixture was stirred for 2 h at 25° C., then concentrated under reduced pressure. The residue was purified by column chromatography (petroleum ether:ethyl acetate=10:1) to give ethyl 2-(3-cyanocyclobutylidene)acetate (28 g, 81%) as a colorless oil.

### Step 3: Preparation of ethyl 2-((1r, 3r)-1-(3-((tert-butoxycarbonyl)amino)phenyl)-3-cyanocyclobutyl)acetate and ethyl 2-((1s, 3s)-1-(3-((tert-butoxycarbonyl)amino)phenyl)-3-cyanocyclobutyl)acetate

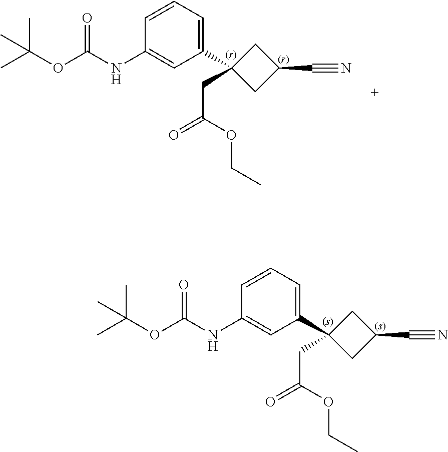 [SMILES: CCOC(=O)C[C@]1(c2cccc(NC(=O)OC(C)(C)C)c2)C[C@H](C#N)C1 ; CCOC(=O)C[C@]1(c2cccc(NC(=O)OC(C)(C)C)c2)C[C@@H](C#N)C1]

To a solution of ethyl 2-(3-cyanocyclobutylidene)acetate (3.30 g, 20 mmol) in dioxane (40 mL) was added [3-(tertbutoxycarbonylamino) phenyl]boronic acid (7.10 g, 30 mmol), 1.5 M potassium hydroxide solution (17.3 mL) and chlororhodium (1Z,5Z)-cycloocta-1,5-diene (493 mg, 1.0 mmol). The mixture was degassed and purged with nitrogen for three times. Then the mixture was stirred for 10 h at 25° C., poured into saturate ammonium chloride (200 mL) and extracted with ethyl acetate (200 mL). The organic layer was washed with brine (100 mL), dried over anhydrous sodium sulfate, filtered, and concentrated under reduced pressure. The residue was purified by column chromatography (petroleum ether:ethyl acetate=10:1 to 5:1) to give ethyl 2-((1r, 3r)-1-(3-((tert-butoxycarbonyl)amino)phenyl)-3-cyanocyclobutyl)acetate (2.6 g, 36%) as a yellow solid. MS (ESI) m/z: 303.1 [M-56+H]⁺; ¹H NMR (400 MHz, CDCl₃) δ 7.38 (s, 1H), 7.28 (d, J=7.6 Hz, 1H), 7.21-7.15 (m, 1H), 6.93 (dd, J=1.2, 7.6 Hz, 1H), 6.53 (s, 1H), 4.01 (q, J=7.2 Hz, 2H), 3.04-2.92 (m, 3H), 2.86 (s, 2H), 2.85-2.78 (m, 2H), 1.54 (s, 9H), 1.15 (t, J=7.2 Hz, 3H). The residue was further purification by prep-HPLC (column: Phenomenex Luna C18 (250×70 mm, 10 um); mobile phase: [water(FA)-acetonitrile]; B %: 55%-80%, 21 min) to give ethyl 2-((1s, 3s)-1-(3-((tert-butoxycarbonyl)amino)phenyl)-3-cyanocyclobutyl)acetate (2.60 g, 36%) as a yellow solid. ¹H NMR (400 MHz, CDCl₃) δ 7.27-7.19 (m, 2H), 7.19-7.12 (m, 1H), 6.78 (dd, J=1.2, 7.6 Hz, 1H), 6.64 (br s, 1H), 3.99 (q, J=7.2 Hz, 2H), 3.25 (quin, J=9.2 Hz, 1H), 2.94-2.87 (m, 2H), 2.86-2.79 (m, 2H), 2.73 (s, 2H), 1.53 (s, 9H), 1.11 (t, J=7.2 Hz, 3H).

### Step 4: Preparation of 2-[1-[3-(tert-butoxycarbonylamino)phenyl]-3-cyano-cyclobutyl]acetic acid

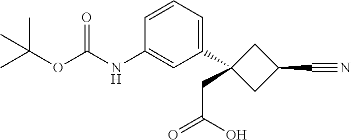 [SMILES: CC(C)(C)OC(=O)Nc1cccc([C@]2(CC(=O)O)C[C@@H](C#N)C2)c1]

To a solution of ethyl 2-[1-[3-(tert-butoxycarbonylamino)phenyl]-3-cyano-cyclobutyl]acetate (2.10 g, 5.9 mmol) in tetrahydrofuran (20 mL), water (8 mL) and methanol (4 mL) was added lithium hydroxide (307 mg, 7.3 mmol). The mixture was stirred for 1 h at 25° C., then concentrated under reduced pressure. The residue was diluted with water (10 mL) and treated with 5% aqueous critic acid until pH=4. Then the mixture was filtered to give 2-[1-[3-(tert-butoxycarbonylamino)phenyl]-3-cyano-cyclobutyl]acetic acid (1.8 g, 93%) as a white solid, which was used in the next step directly. ¹H NMR (400 MHz, MeOD-d₄) δ 7.42 (s, 1H), 7.29-7.20 (m, 2H), 7.03-6.95 (m, 1H), 3.17-3.06 (m, 1H), 2.99-2.90 (m, 2H), 2.85 (s, 2H), 2.83-2.76 (m, 2H), 1.54 (s, 9H).

### Step 5: Preparation of tert-butyl N-[3-[3-cyano-1-[(4-methyl-5-sulfanyl-1,2,4-triazol-3-yl)methyl]cyclobutyl]phenyl]carbamate

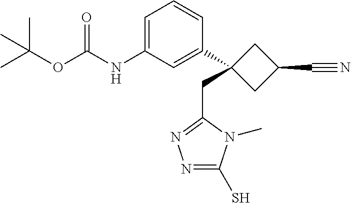 [SMILES: Cn1c(S)nnc1C[C@]1(c2cccc(NC(=O)OC(C)(C)C)c2)C[C@H](C#N)C1]

To a solution of 2-[1-[3-(tert-butoxycarbonylamino)phenyl]-3-cyano-cyclobutyl]acetic acid (1.80 g, 5.5 mmol) in dimethylformamide (10 mL) was added diisopropylethylamine (1.90 mL, 10.9 mmol), o-(7-azabenzotriazol-1-yl)-N,N,N′,N′-tetramethyluronium hexafluorophosphate (2.69 g, 7.1 mmol) and 1-amino-3-methylthiourea (859 mg, 8.2 mmol). The mixture was stirred for 4 h at 25° C. Then to the mixture was added 1 M sodium hydroxide (11.44 mL) and stirred for 10 h at 50° C. The mixture was poured into saturated ammonium chloride (50 mL) and filtered to give tert-butyl N-[3-[3-cyano-1-[(4-methyl-5-sulfanyl-1,2,4-triazol-3-yl)methyl]cyclobutyl]phenyl]carbamate (2.1 g, 96%) as a white solid, which was used in the next step directly. MS (ESI) m/z: 399.9 [M+H]⁺; ¹H NMR (400 MHz, CDCl₃) δ 11.39 (br s, 1H), 7.34 (br s, 1H), 7.26-7.19 (m, 1H), 7.10 (dd, J=1.2, 8.0 Hz, 1H), 6.73 (br s, 1H), 6.64 (d, J=8.4 Hz, 1H), 3.23 (s, 2H), 2.98-2.87 (m, 5H), 2.72 (s, 3H), 1.53 (s, 9H).

### Step 6: Preparation of tert-butyl N-[3-[3-cyano-1-[(4-methyl-1,2,4-triazol-3-yl)methyl]cyclobutyl]phenyl]carbamate

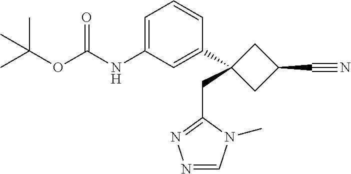 [SMILES: Cn1cnnc1C[C@]1(c2cccc(NC(=O)OC(C)(C)C)c2)C[C@H](C#N)C1]

To a solution of tert-butyl N-[3-[3-cyano-1-[(4-methyl-5-sulfanyl-1,2,4-triazol-3-yl)methyl]cyclobutyl]phenyl]carbamate (2.10 g, 5.3 mmol) in dichloromethane (50 mL) was added acetic acid (4.21 mL, 73 mmol) and 30% hydrogen peroxide solution (1.7 mL, 17 mmol) at 0° C. The mixture was stirred at 25° C. for 2 h, then diluted with dichloromethane (50 mL) and washed with water (100 mL). The organic layer was further washed with saturated sodium sulfite solution (50 mL×2) then brine (50 mL), dried over anhydrous sodium sulfate, filtered, and concentrated under reduced pressure. The residue was purified by column chromatography (dichloromethane:methanol=30:1) to give tert-butyl N-[3-[3-cyano-1-[(4-methyl-1,2,4-triazol-3-yl)methyl]cyclobutyl]phenyl]carbamate (1.80 g, 93%) as a brown solid. MS (ESI) m/z: 368.2 [M+H]⁺; ¹H NMR (400 MHz, CDCl₃) δ 7.85 (s, 1H), 7.31 (d, J=8.0 Hz, 1H), 7.18 (t, J=8.0 Hz, 1H), 6.99 (t, J=2.0 Hz, 1H), 6.78-6.55 (m, 2H), 3.51 (d, J=0.8 Hz, 1H), 3.10-2.87 (m, 6H), 2.73 (s, 3H), 1.53 (s, 9H).

### Step 7: Preparation of 3-(3-aminophenyl)-3-[(4-methyl-1,2,4-triazol-3-yl)methyl]cyclobutanecarbonitrile

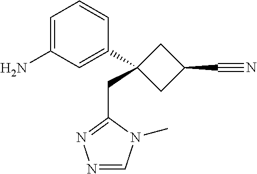 [SMILES: Cn1cnnc1C[C@]1(c2cccc(N)c2)C[C@H](C#N)C1]

To a solution of tert-butyl N-[3-[3-cyano-1-[(4-methyl-1,2,4-triazol-3-yl)methyl]cyclobutyl]phenyl]carbamate (1.00 g, 2.7 mmol) in dichloromethane (5 mL) was added trifluoroacetic acid (10.0 mL, 135 mmol). The mixture was stirred at 25° C. for 0.5 h, then the pH was adjusted to 8 by saturated sodium bicarbonate solution and the aqueous layer was extracted with dichloromethane (10 mL×5). The organic layer was concentrated under reduced pressure. The residue was purified by column chromatography (petroleum ether/ethyl acetate=10/1 to 1/1) to give 3-(3-aminophenyl)-3-[(4-methyl-1,2,4-triazol-3-yl)methyl]cyclobutanecarbonitrile (600 mg, 82%) as a yellow oil.

### Intermediate 6: 3-[3-[6-(2,7-diazaspiro[3.5]nonan-2-yl)-1-oxo-4-(trifluoromethyl)isoindolin-2-yl]phenyl]-3-[(4-methyl-1,2,4-triazol-3-yl)methyl]cyclobutanecarbonitrile

### Step 1: Preparation of 3-[3-[6-bromo-1-oxo-4-(trifluoromethyl)isoindolin-2-yl]phenyl]-3-[(4-methyl-1,2,4-triazol-3-yl)methyl]cyclobutanecarbonitrile

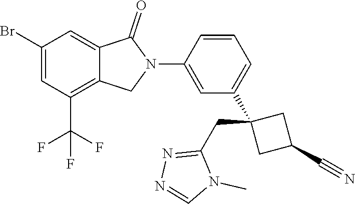 [SMILES: Cn1cnnc1C[C@]1(c2cccc(N3Cc4c(cc(Br)cc4C(F)(F)F)C3=O)c2)C[C@H](C#N)C1]

To a solution of 3-(3-aminophenyl)-3-[(4-methyl-1,2,4-triazol-3-yl)methyl]cyclobutanecarbonitrile (400 mg, 1.5 mmol) and methyl 5-bromo-2-(bromomethyl)-3-(trifluoromethyl)benzoate (585 mg, 1.6 mmol) in acetonitrile (16 mL) and water (8 mL) was added silver nitrate (360 mg, 2.1 mmol) in water (2 mL) at 0° C. The reaction was stirred for 12 h at 25° C., then solid sodium bicarbonate was added until pH=8. The mixture was then filtered through Celite, washed with acetonitrile (50 mL) followed by dichloromethane:ethyl acetate mixture (9:1, 50 mL). The organic layer was separated, dried over sodium sulfate, filtered, and concentrated. The residue was purified by column chromatography (dichloromethane:methanol=1:0 to 10:1) to afford 3-[3-[6-bromo-1-oxo-4-(trifluoromethyl)isoindolin-2-yl]phenyl]-3-[(4-methyl-1,2,4-triazol-3-yl)methyl]cyclobutanecarbonitrile (540 mg, 68%) as a yellow solid. MS (ESI) m/z: 531.8 [M+H]⁺.

### Step 2: Preparation of tert-butyl 2-[2-[3-[3-cyano-1-[(4-methyl-1,2,4-triazol-3-yl)methyl]cyclobutyl]phenyl]-3-oxo-7-(trifluoromethyl)isoindolin-5-yl]-2,7-diazaspiro[3.5]nonane-7-carboxylate

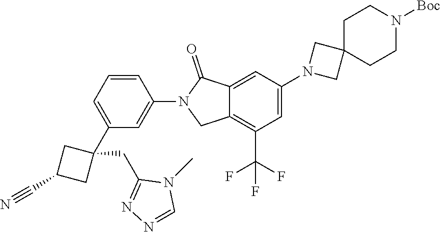 [SMILES: Cn1cnnc1C[C@]1(c2cccc(N3Cc4c(cc(N5CC6(CCN(C(=O)OC(C)(C)C)CC6)C5)cc4C(F)(F)F)C3=O)c2)C[C@H](C#N)C1]

To a solution of 3-[3-[6-bromo-1-oxo-4-(trifluoromethyl)isoindolin-2-yl]phenyl]-3-[(4-methyl-1,2,4-triazol-3-yl)methyl]cyclobutanecarbonitrile (150 mg, 0.3 mmol) and tert-butyl 2,7-diazaspiro[3.5]nonane-7-carboxylate (128 mg, 0.6 mmol) in dioxane (6 mL) was added cesium carbonate (276. mg, 0.8 mmol) and XPhos Pd G4 (73 mg, 0.08 mmol). The mixture was stirred at 90° C. for 12 h. Water (30 mL) was poured into the mixture and stirred for 1 min. The aqueous phase was extracted with ethyl acetate (10 mL×3). The combined organic phase was washed with brine (10 mL×2), dried over anhydrous sodium sulfate, filtered, and concentrated under vacuum. The residue was purified by column chromatography (dichloromethane:methanol=1:0 to 20:1) to afford tert-butyl 2-[2-[3-[3-cyano-1-[(4-methyl-1,2,4-triazol-3-yl)methyl]cyclobutyl]phenyl]-3-oxo-7-(trifluoromethyl)isoindolin-5-yl]-2,7-diazaspiro[3.5]nonane-7-carboxylate (170 mg, 88%) as a yellow solid. MS (ESI) m/z: 676.4 [M+H]⁺.

### Step 3: Preparation of 3-[3-[6-(2,7-diazaspiro[3.5]nonan-2-yl)-1-oxo-4-(trifluoromethyl)isoindolin-2-yl]phenyl]-3-[(4-methyl-1,2,4-triazol-3-yl)methyl]cyclobutanecarbonitrile

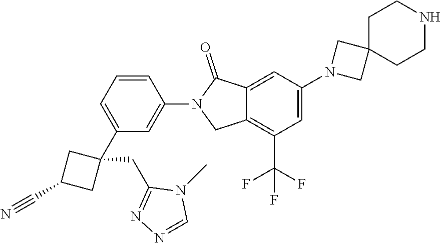 [SMILES: Cn1cnnc1C[C@]1(c2cccc(N3Cc4c(cc(N5CC6(CCNCC6)C5)cc4C(F)(F)F)C3=O)c2)C[C@H](C#N)C1]

To a solution of tert-butyl 2-[2-[3-[3-cyano-1-[(4-methyl-1,2,4-triazol-3-yl)methyl]cyclobutyl]phenyl]-3-oxo-7-(trifluoromethyl)isoindolin-5-yl]-2,7-diazaspiro[3.5]nonane-7-carboxylate (80 mg, 0.1 mmol) in dichloromethane (1.5 mL) was added trifluoroacetic acid (500 μL, 6.8 mmol). The mixture was stirred at 25° C. for 0.5 h, then concentrated to afford 3-[3-[6-(2,7-diazaspiro[3.5]nonan-2-yl)-1-oxo-4-(trifluoromethyl) isoindolin-2-yl]phenyl]-3-[(4-methyl-1,2,4-triazol-3-yl)methyl]cyclobutanecarbonitrile trifluoroacetate (80 mg, 97%) as a yellow oil. MS (ESI) m/z: 576.2 [M+H]⁺.

### Intermediate 7: 6-bromo-2-[3-[3-[(4-methyl-1,2,4-triazol-3-yl)methyl]oxetan-3-yl]phenyl]-4-(trifluoromethyl)indazole

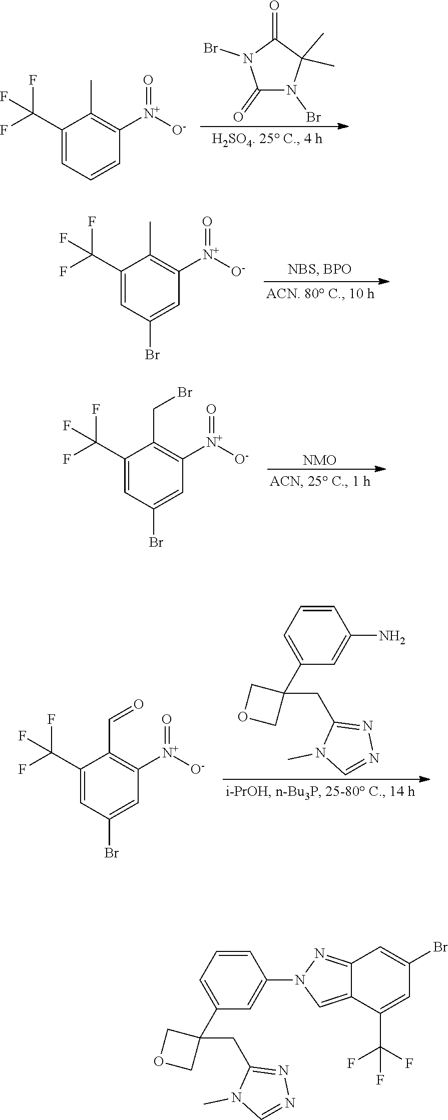

### Step 1: Preparation of 5-bromo-2-methyl-1-nitro-3-(trifluoromethyl)benzene

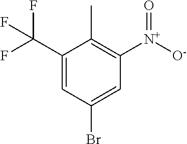

To a solution of 2-methyl-1-nitro-3-(trifluoromethyl)benzene (9.0 g, 44 mmol) in sulfuric acid (135 mL) was slowly added 1,3-dibromo-5,5-dimethyl-imidazolidine-2,4-dione (7.53 g, 26 mmol). The mixture was stirred at 25° C. for 4 h, then diluted with ice water (300 mL) and extracted with ethyl acetate (250 mL×2). The combined organic layer was concentrated under reduced pressure. The residue was purified by silica gel column chromatography (100% petroleum ether) to give 5-bromo-2-methyl-1-nitro-3-(trifluoromethyl)benzene (11.00 g, 88%) as a yellow oil. ¹H NMR (400 MHz, CDCl₃) δ 7.97 (d, J=1.6 Hz, 1H), 7.92 (s, 1H), 2.44 ppm (d, J=1.2 Hz, 3H).

### Step 2: Preparation of 5-bromo-2-(bromomethyl)-1-nitro-3-(trifluoromethyl)benzene

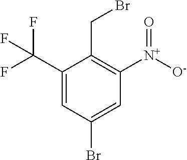 [SMILES: O=[N+]([O-])c1cc(Br)cc(C(F)(F)F)c1CBr]

To a solution of 5-bromo-2-methyl-1-nitro-3-(trifluoromethyl)benzene (1.00 g, 3.5 mmol) and benzoyl peroxide (85 mg, 0.4 mmol) in acetonitrile (10 mL) was added N-bromosuccinimide (658 mg, 3.7 mmol). The mixture was stirred at 80° C. for 10 h under nitrogen atmosphere, then concentrated under reduced pressure. The residue was purified by silica gel column chromatography (petroleum ether/ethyl acetate=1/0 to 30/1) to give 5-bromo-2-(bromomethyl)-1-nitro-3-(trifluoromethyl)benzene (1.00 g, 78%) as a yellow oil.

### Step 3: Preparation of 4-bromo-2-nitro-6-(trifluoromethyl)benzaldehyde

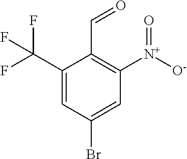 [SMILES: O=Cc1c([N+](=O)[O-])cc(Br)cc1C(F)(F)F]

To a solution of 5-bromo-2-(bromomethyl)-1-nitro-3-(trifluoromethyl)benzene (1.00 g, 2.8 mmol) in acetonitrile (8 mL) was added 4-methyl-4-oxidomorpholin-4-ium (678 mg, 5.8 mmol). The mixture was stirred at 25° C. for 1 h, then ethyl acetate (20 mL) was added and the mixture was stirred for 10 min. The suspension was filtered and concentrated under reduced pressure. The residue was purified by silica gel column chromatography (petroleum ether/ethyl acetate=1/0 to 10/1) to afford 4-bromo-2-nitro-6-(trifluoromethyl)benzaldehyde (580 mg, 71%) as a white solid. ¹H NMR (400 MHz, DMSO-d₆) δ 10.43 (d, J=2.4 Hz, 1H), 8.77 (d, J=1.6 Hz, 1H), 8.58 ppm (d, J=1.2 Hz, 1H).

### Step 4: Preparation of 6-bromo-2-[3-[3-[(4-methyl-1,2,4-triazol-3-yl)methyl]oxetan-3-yl]phenyl]-4-(trifluoromethyl)indazole

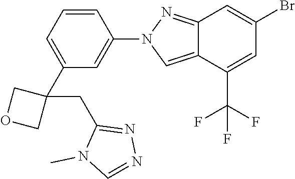 [SMILES: Cn1cnnc1CC1(c2cccc(-n3cc4c(C(F)(F)F)cc(Br)cc4n3)c2)COC1]

A mixture of 4-bromo-2-nitro-6-(trifluoromethyl)benzaldehyde (500 mg, 1.7 mmol) and 3-[3-[(4-methyl-1,2,4-triazol-3-yl)methyl]oxetan-3-yl]aniline (410 mg, 1.7 mmol) in isopropanol (5 mL) was degassed and purged with nitrogen, then stirred at 80° C. for 4 h under nitrogen atmosphere, followed by the addition of tributylphosphane (1.02 g, 5 mmol) at 25° C. The resulting mixture was stirred at 80° C. for 10 h under nitrogen. The mixture was concentrated and the residue was purified by silica gel column chromatography (petroleum ether/ethyl acetate=1/1 to 0/1 then dichloromethane:methanol=20:1) to afford 6-bromo-2-[3-[3-[(4-methyl-1,2,4-triazol-3-yl)methyl]oxetan-3-yl]phenyl]-4-(trifluoromethyl)indazole (300 mg, 36%) as a yellow oil. MS (ESI) m/z: 494.0 [M+H]⁺.

### Intermediate 8: 2-[3-[3-[(4-methyl-1,2,4-triazol-3-yl)methyl]oxetan-3-yl]phenyl]-4-(trifluoromethyl)indazole-6-carbaldehyde

### Step 1: 2-[3-[3-[(4-methyl-1,2,4-triazol-3-yl)methyl]oxetan-3-yl]phenyl]-4-(trifluoromethyl)-6-vinyl-indazole

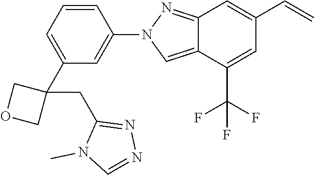 [SMILES: C=Cc1cc(C(F)(F)F)c2cn(-c3cccc(C4(Cc5nncn5C)COC4)c3)nc2c1]

To a solution of 6-bromo-2-[3-[3-[(4-methyl-1,2,4-triazol-3-yl)methyl]oxetan-3-yl]phenyl]-4-(trifluoromethyl)indazole (280 mg, 0.6 mmol) and potassium vinyltrifluoroborate (457 mg, 3.4 mmol) in 1,4-dioxane (5 mL) and water (0.5 mL) was added sodium acetate (140 mg, 1.7 mmol) and [1,1′-bis(diphenylphosphino)ferrocene]dichloropalladium(II) (42 mg, 0.06 mmol). The mixture was stirred at 100° C. for 10 h, then concentrated under reduced pressure. The residue was purified by silica gel column chromatography (dichloromethane:methanol=50/1 to 20/1) to afford 2-[3-[3-[(4-methyl-1,2,4-triazol-3-yl)methyl]oxetan-3-yl]phenyl]-4-(trifluoromethyl)-6-vinyl-indazole (165 mg, 66%) as an off white oil. MS (ESI) m/z: 440.1 [M+H]⁺.

### Step 2: Preparation of 2-[3-[3-[(4-methyl-1,2,4-triazol-3-yl)methyl]oxetan-3-yl]phenyl]-4-(trifluoromethyl)indazole-6-carbaldehyde

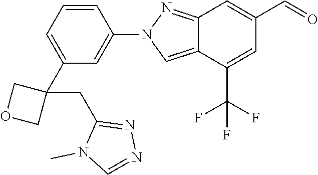 [SMILES: Cn1cnnc1CC1(c2cccc(-n3cc4c(C(F)(F)F)cc(C=O)cc4n3)c2)COC1]

To a solution of 2-[3-[3-[(4-methyl-1,2,4-triazol-3-yl)methyl]oxetan-3-yl]phenyl]-4-(trifluoromethyl)-6-vinyl-indazole (165 mg, 0.4 mmol) in 1,4-dioxane (2 mL) and water (0.4 mL) was added 2,6-dimethylpyridine (80 mg, 0.8 mmol), potassium osmate(VI) dihydrate (14 mg, 0.04 mmol) and sodium periodate (321 mg, 1.5 mmol). The mixture was stirred at 25° C. for 2 h, then filtered. The filtrate was diluted with saturated sodium thiosulphate solution (10 mL) and extracted with ethyl acetate (10 mL×2). The combined organic layers were concentrated under reduced pressure. The residue was purified by silica gel column chromatography (petroleum ether/ethyl acetate=1/0 to 8/1) to afford 2-[3-[3-[(4-methyl-1,2,4-triazol-3-yl)methyl]oxetan-3-yl]phenyl]-4-(trifluoromethyl)indazole-6-carbaldehyde (120 mg, 72%) as an off white oil. MS (ESI) m/z: 442.3 [M+H]⁺.

### Example 1: Exemplary synthesis of 3-{4-fluoro-5-[4-({2-[2-(3-{3-[(4-methyl-4H-1,2,4-triazol-3-yl)methyl]oxetan-3-yl}phenyl)-3-oxo-7-(trifluoromethyl)-2,3-dihydro-1H-isoindol-5-yl]-2,7-diazaspiro[3.5]nonan-7-yl}methyl)piperidin-1-yl]-1-oxo-2,3-dihydro-1H-isoindol-2-yl}piperidine-2,6-dione

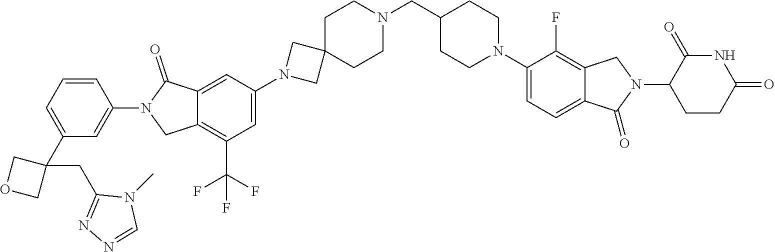 [SMILES: Cn1cnnc1CC1(c2cccc(N3Cc4c(cc(N5CC6(CCN(CC7CCN(c8ccc9c(c8F)CN(C8CCC(=O)NC8=O)C9=O)CC7)CC6)C5)cc4C(F)(F)F)C3=O)c2)COC1]

To a solution of 6-(2,7-diazaspiro[3.5]nonan-2-yl)-2-[3-[3-[(4-methyl-1,2,4-triazol-3-yl)methyl]oxetan-3-yl]phenyl]-4-(trifluoromethyl)isoindolin-1-one (50 mg, 0.09 mmol) and 1-[2-(2,6-dioxo-3-piperidyl)-4-fluoro-1-oxo-isoindolin-5-yl]piperidine-4-carbaldehyde (34 mg, 0.09 mmol) in dichloromethane (2 mL) was added triethylamine (18 mg, 0.2 mmol) and chloro(triisopropoxy)titanium (24 mg, 0.09 mmol). The mixture was stirred at 25° C. for 10 h. Sodium triacetoxyborohydride (39 mg, 0.2 mmol) was added and the mixture was stirred for 1 h, then concentrated under reduced pressure. The residue was purified by preparative thin layer chromatography (dichloromethane:methanol=10:1). The crude product was further purified by preparative HPLC (column: Phenomenex Synergi C18 150*25 mm*10 um; mobile phase: [formic acid in water-acetonitrile]; B %: 18%-38%, 9 min) to give 3-[4-fluoro-5-[4-[[2-[2-[3-[3-[(4-methyl-1,2,4-triazol-3-yl)methyl]oxetan-3-yl]phenyl]-3-oxo-7-(trifluoromethyl)isoindolin-5-yl]-2,7-diazaspiro[3.5]nonan-7-yl]methyl]-1-piperidyl]-1-oxo-isoindolin-2-yl]piperidine-2,6-dione (9.6 mg, 12%) as a white solid. MS (ESI) m/z: 910.5 [M+H]⁺; ¹H NMR (400 MHz, DMSO-d₆) δ 10.98 (s, 1H), 8.19 (s, 1H), 7.88 (d, J=7.6 Hz, 1H), 7.47 (d, J=8.4 Hz, 1H), 7.38-7.32 (m, 2H), 7.17 (t, J=8.0 Hz, 1H), 6.94 (s, 2H), 6.75 (d, J=7.6 Hz, 1H), 5.08 (dd, J=4.8, 13.2 Hz, 1H), 4.98-4.93 (m, 4H), 4.89 (d, J=6.0 Hz, 2H), 4.53-4.45 (m, 1H), 4.35-4.27 (m, 1H), 3.70 (s, 4H), 3.51 (s, 4H), 3.30-3.29 (m, 2H), 2.91 (s, 4H), 2.83-2.73 (m, 3H), 2.62-2.60 (m, 1H), 2.18 (d, J=6.8 Hz, 2H), 2.00-1.95 (m, 1H), 1.84 (s, 2H), 1.81-1.78 (m, 2H), 1.78 (s, 2H), 1.76-1.51 (m, 2H), 1.35-1.00 (m, 3H).

### Example 2: Exemplary synthesis of 3-[4-fluoro-1-oxo-5-(4-{[(1r, 4r)-4-[(4-{[2-(3-{3-[(4-methyl-4H-1,2,4-triazol-3-yl)methyl]oxetan-3-yl}phenyl)-3-oxo-7-(trifluoromethyl)-2,3-dihydro-1H-isoindol-5-yl]methyl}piperazin-1-yl)methyl]cyclohexyl]oxy}piperidin-1-yl)-2,3-dihydro-1H-isoindol-2-yl]piperidine-2,6-dione

### Step 1: Preparation of tert-butyl 4-[[4-[[1-[2-(2,6-dioxo-3-piperidyl)-4-fluoro-1-oxo-isoindolin-5-yl]-4-piperidyl]oxy]cyclohexyl]methyl]piperazine-1-carboxylate

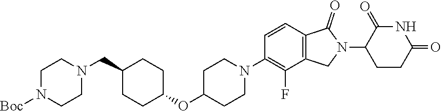 [SMILES: CC(C)(C)OC(=O)N1CCN(C[C@H]2CC[C@H](OC3CCN(c4ccc5c(c4F)CN(C4CCC(=O)NC4=O)C5=O)CC3)CC2)CC1]

To a stirred solution of tert-butyl piperazine-1-carboxylate hydrochloride (213 mg, 0.9 mmol) in N,N-dimethylformamide (5 mL) was added nitromethyl morpholine (193 mg, 1.9 mmol) and 4-[[1-[2-(2,6-dioxo-3-piperidyl)-4-fluoro-1-oxo-isoindolin-5-yl]-4-piperidyl]oxy]cyclohexanecarbaldehyde (300 mg, 0.6 mmol) at 0° C. Then sodium triacetoxyborohydride (270 mg, 1.3 mmol) was added and the mixture was stirred at 25° C. for 10 h. The reaction mixture was partitioned between water (30 mL) and ethyl acetate (10 mL). The organic phase was separated, washed with brine (20 mL), dried over dried over anhydrous sodium sulfate, filtered, and concentrated. The residue was purified by preparative HPLC (column: Phenomenex Luna C18 150×40 mm×15 um; mobile phase: [formic acid in water-acetonitrile]; B %: 12%-42%, 10 min) to give tert-butyl 4-[[4-[[1-[2-(2,6-dioxo-3-piperidyl)-4-fluoro-1-oxo-isoindolin-5-yl]-4-piperidyl]oxy]cyclohexyl]methyl]piperazine-1-carboxylate (50 mg, 12%) as a white solid. MS (ESI) m/z: 642.4 [M+H]⁺.

### Step 2: Preparation of 3-[4-fluoro-1-oxo-5-[4-[4-(piperazin-1-ylmethyl)cyclohexoxy]-1-piperidyl]isoindolin-2-yl]piperidine-2,6-dione

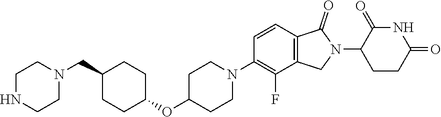 [SMILES: O=C1CCC(N2Cc3c(ccc(N4CCC(O[C@H]5CC[C@H](CN6CCNCC6)CC5)CC4)c3F)C2=O)C(=O)N1]

To a solution of tert-butyl 4-[[4-[[1-[2-(2,6-dioxo-3-piperidyl)-4-fluoro-1-oxo-isoindolin-5-yl]-4-piperidyl]oxy]cyclohexyl]methyl]piperazine-1-carboxylate (50 mg, 0.08 mmol) in dichloromethane (2 mL) was added trifluoroacetic acid (27.0 mg, 234 mmol). The mixture was stirred at 25° C. for 1 h, then concentrated under reduced pressure to give 3-[4-fluoro-1-oxo-5-[4-[4-(piperazin-1-ylmethyl)cyclohexoxy]-1-piperidyl]isoindolin-2-yl]piperidine-2,6-dione trifluoroacetate (50 mg, 98%) as a colorless oil, which was used in the next step directly. MS (ESI) m/z: 542.3 [M+H]⁺.

### Step 3: Preparation of 3-[4-fluoro-5-[4-[4-[[4-[[2-[3-[3-[(2-methyl-1,2,4-triazol-3-yl)methyl]oxetan-3-yl]phenyl]-3-oxo-7-(trifluoromethyl)isoindolin-5-yl]methyl]piperazin-1-yl]methyl]cyclohexoxy]-1-piperidyl]-1-oxo-isoindolin-2-yl]piperidine-2,6-dione

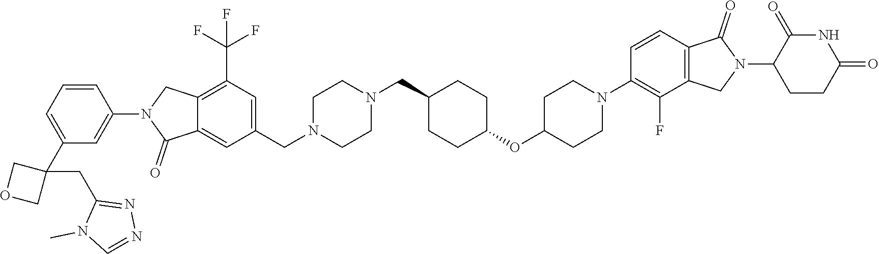 [SMILES: Cn1cnnc1CC1(c2cccc(N3Cc4c(cc(CN5CCN(C[C@H]6CC[C@H](OC7CCN(c8ccc9c(c8F)CN(C8CCC(=O)NC8=O)C9=O)CC7)CC6)CC5)cc4C(F)(F)F)C3=O)c2)COC1]

To a solution of 3-[4-fluoro-1-oxo-5-[4-[4-(piperazin-1-ylmethyl)cyclohexoxy]-1-piperidyl]isoindolin-2-yl]piperidine-2,6-dione trifluoroacetate (50 mg, 0.08 mmol) and 2-[3-[3-[(4-methyl-1,2,4-triazol-3-yl)methyl]oxetan-3-yl]phenyl]-3-oxo-7-(trifluoromethyl)isoindoline-5-carbaldehyde (35 mg, 0.08 mmol) in dichloromethane (3 mL) was added triethylamine (23 mg, 0.23 mmol) and chloro(triisopropoxy)titanium (20 mg, 0.08 mmol). The mixture was stirred at 25° C. for 10 h. Sodium triacetoxyborohydride (48 mg, 0.23 mmol) was added and the mixture was stirred for 1 h, then concentrated under reduced pressure. The residue was purified by preparative HPLC (column: Phenomenex Synergi C18 150×25 mm×10 um; mobile phase: [water(formic acid)-acetonitrile]; B %: 18%-38%, 10 min) to give 3-[4-fluoro-5-[4-[4-[[4-[[2-[3-[3-[(2-methyl-1,2,4-triazol-3-yl)methyl]oxetan-3-yl]phenyl]-3-oxo-7-(trifluoromethyl)isoindolin-5-yl]methyl]piperazin-1-yl]methyl]cyclohexoxy]-1-piperidyl]-1-oxo-isoindolin-2-yl]piperidine-2,6-dione (22.5 mg, 30%) as a white solid. MS (ESI) m/z: 982.6 [M+H]⁺; ¹H NMR (400 MHz, DMSO-d₆) δ 10.98 (s, 1H), 8.19 (s, 1H), 7.98 (s, 1H), 7.93 (s, 1H), 7.89 (dd, J=1.6, 8.0 Hz, 1H), 7.46 (d, J=8.0 Hz, 1H), 7.40 (s, 1H), 7.36 (t, J=8.0 Hz, 1H), 7.17 (t, J=8.0 Hz, 1H), 6.77 (d, J=7.6 Hz, 1H), 5.10 (s, 2H), 5.09-5.03 (m, 1H), 4.97 (d, J=6.0 Hz, 2H), 4.89 (d, J=6.0 Hz, 2H), 4.48 (d, J=17.2 Hz, 1H), 4.31 (d, J=16.8 Hz, 1H), 3.69 (s, 2H), 3.66-3.57 (m, 1H), 3.52 (s, 2H), 3.36 (s, 1H), 3.30 (s, 3H), 3.00-2.92 (m, 2H), 2.91 (s, 4H), 2.63-2.55 (m, 1H), 2.53-2.52 (m, 2H), 2.43 (dd, J=4.0, 8.4 Hz, 5H), 2.11-2.05 (m, 2H), 2.02-1.85 (m, 6H), 1.79-1.72 (m, 2H), 1.62-1.53 (m, 2H), 1.47-1.38 (m, 1H), 1.19-1.09 (m, 2H), 0.92-0.81 (m, 2H).

### Example 3: Exemplary synthesis of 3-[4-fluoro-1-oxo-5-(4-{[(1r, 4r)-4-[(4-{[(3S)-1-{[2-(3-{3-[(4-methyl-4H-1,2,4-triazol-3-yl)methyl]oxetan-3-yl}phenyl)-3-oxo-7-(trifluoromethyl)-2,3-dihydro-1H-isoindol-5-yl]methyl}piperidin-3-yl]methyl}piperazin-1-yl)methyl]cyclohexyl]oxy}piperidin-1-yl)-2,3-dihydro-1H-isoindol-2-yl]piperidine-2,6-dione

### Step 1: Preparation of benzyl 4-[[(3R)-1-tert-butoxycarbonyl-3-piperidyl]methyl]piperazine-1-carboxylate

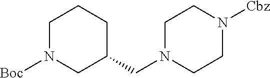 [SMILES: CC(C)(C)OC(=O)N1CCC[C@H](CN2CCN(C(=O)OCc3ccccc3)CC2)C1]

To a solution of tert-butyl (3S)-3-(p-tolylsulfonyloxymethyl)piperidine-1-carboxylate (1.00 g, 2.7 mmol) and benzyl piperazine-1-carboxylate (596 mg, 2.7 mmol) in acetonitrile (10 mL) was added diisopropylethylamine (1.05 g, 8.1 mmol). The mixture was stirred at 80° C. for 10 h, then concentrated under reduced pressure. The residue was purified by column chromatography (petroleum ether/ethyl acetate=20/1 to 5/1) to give benzyl 4-[[(3R)-1-tert-butoxycarbonyl-3-piperidyl]methyl]piperazine-1-carboxylate (1 g, 88%) as a colorless oil. MS (ESI) m/z: 418.2 [M+H]⁺; ¹H NMR (400 MHz, DMSO-d₆) δ 7.40-7.28 (m, 5H), 5.07 (s, 2H), 3.96-3.69 (m, 2H), 3.39 (s, 4H), 3.31 (s, 2H), 2.83-2.69 (m, 1H), 2.37-2.24 (m, 4H), 1.76-1.65 (m, 1H), 1.64-1.52 (m, 2H), 1.38 (s, 9H), 1.34-1.23 (m, 2H), 1.14-0.97 (m, 1H).

### Step 2: Preparation of tert-butyl (3R)-3-(piperazin-1-ylmethyl)piperidine-1-carboxylate

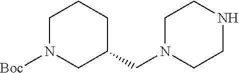 [SMILES: CC(C)(C)OC(=O)N1CCC[C@H](CN2CCNCC2)C1]

To a solution of benzyl 4-[[(3R)-1-tert-butoxycarbonyl-3-piperidyl]methyl]piperazine-1-carboxylate (500 mg, 1.2 mmol) in methanol (10 mL) was added palladium on carbon (100 mg) under nitrogen atmosphere. The suspension was degassed and purged with hydrogen three times, then stirred under hydrogen (15 Psi) at 25° C. for 10 h. The mixture was filtered and the filtrate was concentrated under reduced pressure to give tert-butyl (3R)-3-(piperazin-1-ylmethyl)piperidine-1-carboxylate (310 mg, 91%) as a white solid, which was used in the next step directly. MS (ESI) m/z: 284.3 [M+H]⁺.

### Step 3: Preparation of tert-butyl (3R)-3-[[4-[[4-[[1-[2-(2,6-dioxo-3-piperidyl)-4-fluoro-1-oxo-isoindolin-5-yl]-4-piperidyl]oxy]cyclohexyl]methyl]piperazin-1-yl]methyl]piperidine-1-carboxylate

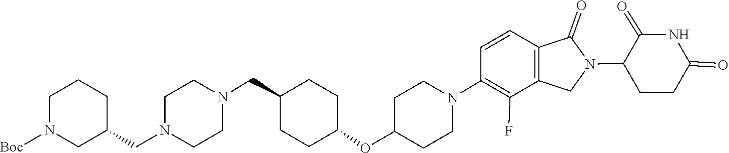 [SMILES: CC(C)(C)OC(=O)N1CCC[C@H](CN2CCN(C[C@H]3CC[C@H](OC4CCN(c5ccc6c(c5F)CN(C5CCC(=O)NC5=O)C6=O)CC4)CC3)CC2)C1]

To a stirred solution of tert-butyl (3R)-3-(piperazin-1-ylmethyl)piperidine-1-carboxylate (121 mg, 0.4 mmol) and 4-[[1-[2-(2,6-dioxo-3-piperidyl)-4-fluoro-1-oxo-isoindolin-5-yl]-4-piperidyl]oxy]cyclohexanecarbaldehyde (100 mg, 0.2 mmol) in dichloromethane (2 mL) was added sodium triacetoxyborohydride (90 mg, 0.4 mmol) at 0° C. The mixture was stirred at 25° C. for 10 h, then concentrated under reduced pressure. The residue was purified by preparative TLC (dichloromethane:methanol=10:1) to give tert-butyl (3R)-3-[[4-[[4-[[1-[2-(2,6-dioxo-3-piperidyl)-4-fluoro-1-oxo-isoindolin-5-yl]-4-piperidyl]oxy]cyclohexyl]methyl]piperazin-1-yl]methyl]piperidine-1-carboxylate (150 mg, 96%) as a white solid. MS (ESI) m/z: 739.4 [M+H]⁺.

### Step 4: Preparation of 3-[4-fluoro-1-oxo-5-[4-[4-[[4-[[(3S)-3-piperidyl]methyl]piperazin-1-yl]methyl]cyclohexoxy]-1-piperidyl]isoindolin-2-yl]piperidine-2,6-dione

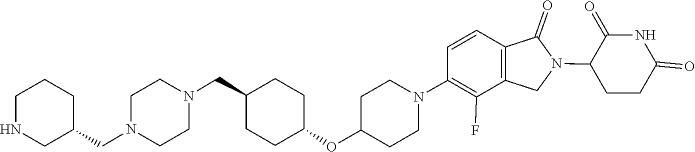 [SMILES: O=C1CCC(N2Cc3c(ccc(N4CCC(O[C@H]5CC[C@H](CN6CCN(C[C@H]7CCCNC7)CC6)CC5)CC4)c3F)C2=O)C(=O)N1]

To a solution of tert-butyl (3R)-3-[[4-[[4-[[1-[2-(2,6-dioxo-3-piperidyl)-4-fluoro-1-oxo-isoindolin-5-yl]-4-piperidyl]oxy]cyclohexyl]methyl]piperazin-1-yl]methyl]piperidine-1-carboxylate (75 mg, 0.1 mmol) in dichloromethane (2 mL) was added trifluoroacetic acid (35 mg, 0.3 mmol). The mixture was stirred at 25° C. for 0.5 h, then concentrated under reduced pressure to give 3-[4-fluoro-1-oxo-5-[4-[4-[[4-[[(3S)-3-piperidyl]methyl]piperazin-1-yl]methyl]cyclohexoxy]-1-piperidyl]isoindolin-2-yl]piperidine-2,6-dione trifluoroacetate (76 mg, 99%) as a colorless oil, which was used in the next step directly. MS (ESI) m/z: 639.6 [M+H]⁺.

### Step 5: Preparation of 3-[4-fluoro-5-[4-[4-[[4-[[(3S)-1-[[2-[3-[3-[(2-methyl-1,2,4-triazol-3-yl)methyl]oxetan-3-yl]phenyl]-3-oxo-7-(trifluoromethyl)isoindolin-5-yl]methyl]-3-piperidyl]methyl]piperazin-1-yl]methyl]cyclohexoxy]-1-piperidyl]-1-oxo-isoindolin-2-yl]piperidine-2,6-dione

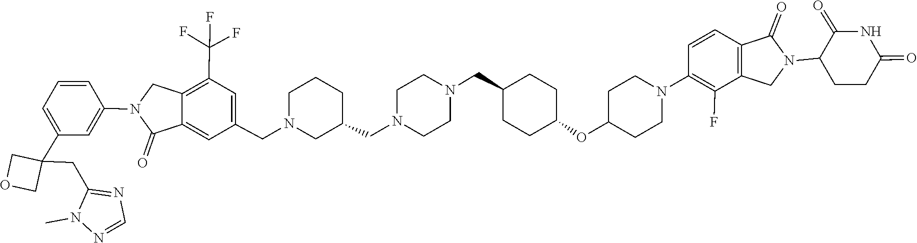 [SMILES: Cn1ncnc1CC1(c2cccc(N3Cc4c(cc(CN5CCC[C@H](CN6CCN(C[C@H]7CC[C@H](OC8CCN(c9ccc%10c(c9F)CN(C9CCC(=O)NC9=O)C%10=O)CC8)CC7)CC6)C5)cc4C(F)(F)F)C3=O)c2)COC1]

To a solution of 3-[4-fluoro-1-oxo-5-[4-[4-[[4-[[(3S)-3-piperidyl]methyl]piperazin-1-yl]methyl]cyclohexoxy]-1-piperidyl]isoindolin-2-yl]piperidine-2,6-dione (73 mg, 0.1 mmol) and 2-[3-[3-[(4-methyl-1,2,4-triazol-3-yl)methyl]oxetan-3-yl]phenyl]-3-oxo-7-(trifluoromethyl)isoindoline-5-carbaldehyde (40 mg, 0.09 mmol) in dichloromethane (3 mL) was added triethylamine (27 mg, 0.3 mmol) and chloro(triisopropoxy)titanium (23 mg, 0.09 mmol). The mixture was stirred at 25° C. for 10 h. Sodium triacetoxyborohydride (56 mg, 0.3 mmol) was added and the mixture was stirred for 1 h, then concentrated under reduced pressure. The residue was purified by preparative HPLC (column: Phenomenex Luna C18 150×25 mm×10 um; mobile phase: [formic acid in water-acetonitrile]; B %: 11%-31%, 10 min) to give 3-[4-fluoro-5-[4-[4-[[4-[[(3S)-1-[[2-[3-[3-[(2-methyl-1,2,4-triazol-3-yl)methyl]oxetan-3-yl]phenyl]-3-oxo-7-(trifluoromethyl)isoindolin-5-yl]methyl]-3-piperidyl]methyl]piperazin-1-yl]methyl]cyclohexoxy]-1-piperidyl]-1-oxo-isoindolin-2-yl]piperidine-2,6-dione (19.4 mg, 19%) as a white solid. MS (ESI) m/z: 1079.8 [M+H]⁺; ¹H NMR (400 MHz, DMSO-d₆) δ 10.98 (s, 1H), 8.19 (s, 1H), 8.09-7.92 (m, 2H), 7.89 (d, J=8.8 Hz, 1H), 7.53-7.44 (m, 1H), 7.42-7.31 (m, 2H), 7.17 (t, J=8.0 Hz, 1H), 6.80 (d, J=7.2 Hz, 1H), 5.16-5.10 (m, 2H), 4.97 (d, J=6.0 Hz, 2H), 4.90 (d, J=6.0 Hz, 2H), 4.48 (d, J=17.2 Hz, 1H), 4.31 (d, J=17.2 Hz, 1H), 3.71-3.57 (m, 2H), 3.55-3.50 (m, 2H), 3.41-3.34 (m, 4H), 2.93 (s, 6H), 2.62 (d, J=6.4 Hz, 8H), 2.47-2.42 (m, 8H), 2.40 (d, J=5.2 Hz, 1H), 1.98 (dd, J=3.2, 8.8 Hz, 2H), 1.96-1.86 (m, 6H), 1.78-1.68 (m, 4H), 1.62-1.49 (m, 4H), 1.19-1.11 (m, 2H), 1.02-0.83 (m, 3H).

### Example 4: Exemplary synthesis of 3-{4-chloro-5-[4-({2-[2-(3-{3-[(4-methyl-4H-1,2,4-triazol-3-yl)methyl]oxetan-3-yl}phenyl)-3-oxo-7-(trifluoromethyl)-2,3-dihydro-1H-isoindol-5-yl]-2,7-diazaspiro[3.5]nonan-7-yl}methyl)piperidin-1-yl]-1-oxo-2,3-dihydro-1H-isoindol-2-yl}piperidine-2,6-dione

### Step 1: Preparation of methyl 2-bromo-4-[4-(dimethoxymethyl)-1-piperidyl]benzoate

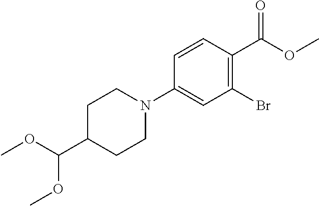

To a solution of 4-(dimethoxymethyl)piperidine (44.41 g, 279 mmol) and methyl 2-bromo-4-fluoro-benzoate (50.0 g, 215 mmol) in dimethyl sulfoxide (500 mL) was added N,N-diisopropylethylamine (55.46 g, 429 mmol). The mixture was stirred at 120° C. for 2 h, then cooled to room temperature. The mixture was diluted with water (1500 mL) and extracted with ethyl acetate (3×500 mL). The combined organic layers were washed with brine (3×1000 mL), dried over anhydrous sodium sulfate, filtered, and concentrated under vacuum. The crude product was triturated with petroleum ether and ethyl acetate (V:V=20:1, 200 mL) to give methyl 2-bromo-4-[4-(dimethoxymethyl)-1-piperidyl]benzoate (64 g, 79%) as a light yellow solid.

### Step 2: Preparation of methyl 4-[4-(dimethoxymethyl)-1-piperidyl]-2-formyl-benzoate

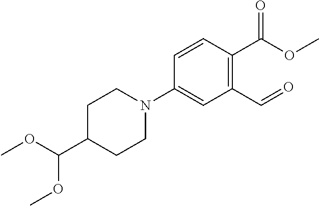

To a solution of methyl 2-bromo-4-[4-(dimethoxymethyl)-1-piperidyl]benzoate (52.0 g, 140 mmol) in N,N-dimethylformamide (500 mL) was added 2-isocyano-2-methyl-propane (23.23 g, 279 mmol), palladium acetate (3.14 g, 14 mmol), tricyclohexylphosphane (3.92 g, 14 mmol), sodium carbonate (14.81 g, 140 mmol) and triethylsilane (48.73 g, 419 mmol). The mixture was stirred at 65° C. for 12 h in the autoclave. The mixture was diluted with water (500 mL) and extracted with ethyl acetate (3×300 mL). The combined organic layers were washed with brine (3×500 mL), dried over anhydrous sodium sulfate, filtered, and concentrated under vacuum. The residue was purified by flash silica gel chromatography (0-15% ethyl acetate/petroleum ether). Then the crude product was triturated with petroleum ether and ethyl acetate (v:v=10:1, 300 mL) to afford methyl 4-[4-(dimethoxymethyl)-1-piperidyl]-2-formyl-benzoate (22 g, 49%) as a light yellow solid. ¹H NMR (400 MHz, CDCl₃) δ 10.74 (s, 1H), 7.90 (d, J=8.8 Hz, 1H), 7.33 (d, J=2.8 Hz, 1H), 7.00 (dd, J=2.8, 8.8 Hz, 1H), 4.06 (d, J=6.4 Hz, 1H), 3.96 (d, J=12.8 Hz, 2H), 3.91 (s, 3H), 3.38 (s, 6H), 2.93-2.82 (m, 2H), 1.86 (d, J=10.0 Hz, 3H), 1.46-1.35 (m, 2H).

### Step 3: Preparation of 3-[5-[4-(dimethoxymethyl)-1-piperidyl]-1-oxo-isoindolin-2-yl]piperidine-2,6-dione

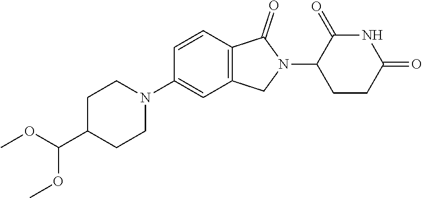 [SMILES: COC(OC)C1CCN(c2ccc3c(c2)CN(C2CCC(=O)NC2=O)C3=O)CC1]

To a solution of 3-aminopiperidine-2,6-dione hydrochloride (12.11 g, 73 mmol) in methanol (400 mL) was added sodium acetate (10.98 g, 134 mmol). The mixture was stirred at 15° C. for 10 min. Then acetic acid (40.18 g, 669 mmol) and methyl 4-[4-(dimethoxymethyl)-1-piperidyl]-2-formyl-benzoate (21.5 g, 67 mmol) was added to the mixture. The mixture was stirred at 15° C. for 20 min, then sodium cyanoborohydride (8.41 g, 134 mmol) was added. The mixture was stirred at 35° C. for 11.5 h. The crude product was poured into ice water (1000 mL) and the pH was adjusted to 8 with saturated sodium bicarbonate solution. The mixture was stirred at 15° C. for 10 min. The mixture was filtered, and the filter cake was washed with water (200 mL) and acetonitrile (2×200 mL), then triturated with ethyl acetate (100 mL) to give 3-[5-[4-(dimethoxymethyl)-1-piperidyl]-1-oxo-isoindolin-2-yl]piperidine-2,6-dione (20 g, 73%) as a light yellow solid. MS (ESI) m/z: 402.2 [M+H]⁺; ¹H NMR (400 MHz, DMSO-d₆) δ 10.93 (s, 1H), 7.49 (d, J=8.4 Hz, 1H), 7.07-7.00 (m, 2H), 5.04 (dd, J=5.2, 13.2 Hz, 1H), 4.35-4.25 (m, 1H), 4.24-4.14 (m, 1H), 4.07 (d, J=6.8 Hz, 1H), 3.89 (d, J=12.8 Hz, 2H), 3.27 (s, 6H), 2.94-2.85 (m, 1H), 2.83-2.72 (m, 2H), 2.63-2.54 (m, 1H), 2.36 (dq, J=4.4, 13.2 Hz, 1H), 2.00-1.91 (m, 1H), 1.80 (dtd, J=3.6, 7.6, 15.2 Hz, 1H), 1.70 (d, J=12.8 Hz, 2H), 1.30 (dq, J=3.6, 12.4 Hz, 2H).

### Step 4: Preparation of 3-(4-chloro-5-(4-(dimethoxymethyl)piperidin-1-yl)-1-oxoisoindolin-2-yl)piperidine-2,6-dione

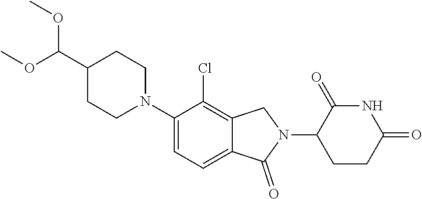 [SMILES: COC(OC)C1CCN(c2ccc3c(c2Cl)CN(C2CCC(=O)NC2=O)C3=O)CC1]

To a solution of 3-(5-(4-(dimethoxymethyl)piperidin-1-yl)-1-oxoisoindolin-2-yl)piperidine-2,6-dione (200 mg, 498 μmol) in dichloromethane (20 mL) and methanol (2 mL) was added N-chlorosuccinimide (99.79 mg, 747 μmol) and trifluoroacetic acid (73.8 μL, 996 μmol). The mixture was stirred at 25° C. for 12 h, then filtered and concentrated under reduced pressure. The residue was purified by preparative HPLC (column: Phenomenex Luna C18 150*25 mm*10 um; mobile phase: [formic acid in water-acetonitrile]; B %: 30%-60%, 10 min) to afford 3-(4-chloro-5-(4-(dimethoxymethyl)piperidin-1-yl)-1-oxoisoindolin-2-yl)piperidine-2,6-dione (60 mg, 27%) as a white solid. MS (ESI) m/z: 436.3 [M+H]⁺.

### Step 5: Preparation of 1-(4-chloro-2-(2,6-dioxopiperidin-3-yl)-1-oxoisoindolin-5-yl)piperidine-4-carbaldehyde

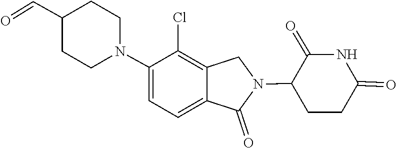 [SMILES: O=CC1CCN(c2ccc3c(c2Cl)CN(C2CCC(=O)NC2=O)C3=O)CC1]

To a solution of 3-(4-chloro-5-(4-(dimethoxymethyl)piperidin-1-yl)-1-oxoisoindolin-2-yl)piperidine-2,6-dione (60 mg, 149 μmol) in dichloromethane (20 mL) was added trifluoroacetic acid (11.07 μL, 149 μmol). The mixture was stirred at 25° C. for 0.5 h, then concentrated under vacuum to give 1-(4-chloro-2-(2,6-dioxopiperidin-3-yl)-1-oxoisoindolin-5-yl)piperidine-4-carbaldehyde trifluoroacetate (75 mg, 99%) as a white solid. MS (ESI) m/z: 390.2 [M+H]⁺.

### Step 6: Preparation of 3-(4-chloro-5-(4-((2-(2-(3-(3-((4-methyl-4H-1,2,4-triazol-3-yl)methyl)oxetan-3-yl)phenyl)-3-oxo-7-(trifluoromethyl)isoindolin-5-yl)-2,7-diazaspiro[3.5]nonan-7-yl)methyl)piperidin-1-yl)-1-oxoisoindolin-2-yl)piperidine-2,6-dione

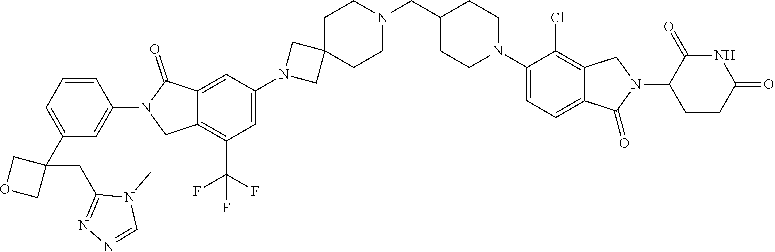 [SMILES: Cn1cnnc1CC1(c2cccc(N3Cc4c(cc(N5CC6(CCN(CC7CCN(c8ccc9c(c8Cl)CN(C8CCC(=O)NC8=O)C9=O)CC7)CC6)C5)cc4C(F)(F)F)C3=O)c2)COC1]

To a solution of 2-(3-(3-((4-methyl-4H-1,2,4-triazol-3-yl)methyl)oxetan-3-yl)phenyl)-6-(2,7-diazaspiro[3.5]nonan-2-yl)-4-(trifluoromethyl)isoindolin-1-one trifluoroacetate (61 mg, 92 μmol) and 1-(4-chloro-2-(2,6-dioxopiperidin-3-yl)-1-oxoisoindolin-5-yl)piperidine-4-carbaldehyde trifluoroacetate (46 mg, 92 μmol) in dichloromethane (1 mL) and dimethylsulfoxide (1 mL) was added N-methylmorphonophosphate (0.5 mL, 4.5 mmol) and sodium triacetoxyborohydride (97.0 mg, 457 μmol). The mixture was stirred at 25° C. for 12 h. The reaction mixture was quenched with water (30 mL) and extracted with dichloromethane (3×10 mL). The combined organic layers were washed with brine (3×10 mL), dried over anhydrous sodium sulfate, filtered, and concentrated. The residue was purified by preparative HPLC (column: Phenomenex Luna C18 150×25 mm×10 um; mobile phase: [formic acid in water-acetonitrile]; B %: 16%-46%, 10 min) to afford 3-(4-chloro-5-(4-((2-(2-(3-(3-((4-methyl-4H-1,2,4-triazol-3-yl)methyl)oxetan-3-yl)phenyl)-3-oxo-7-(trifluoromethyl)isoindolin-5-yl)-2,7-diazaspiro[3.5]nonan-7-yl)methyl)piperidin-1-yl)-1-oxoisoindolin-2-yl)piperidine-2,6-dione (12.9 mg, 14%) as a white solid. MS (ESI) m/z: 926.2 [M+H]⁺; ¹H NMR (400 MHz, DMSO-d₆) δ 11.04-10.91 (m, 1H), 8.18 (s, 1H), 7.91-7.83 (m, 1H), 7.69-7.61 (m, 1H), 7.38-7.30 (m, 2H), 7.26 (d, J=8.4 Hz, 1H), 6.93 (s, 2H), 6.74 (d, J=7.8 Hz, 1H), 5.14-5.06 (m, 1H), 4.98-4.92 (m, 4H), 4.88 (d, J=6.0 Hz, 2H), 4.45-4.36 (m, 1H), 4.31-4.21 (m, 1H), 3.69 (s, 4H), 3.54-3.47 (m, 3H), 2.90 (s, 3H), 2.80-2.69 (m, 2H), 2.58-2.54 (m, 1H), 2.33 (br dd, J=1.8, 3.6 Hz, 4H), 2.22-2.15 (m, 2H), 2.05-1.92 (m, 2H), 1.88-1.64 (m, 8H), 1.37-1.21 (m, 3H).

### Example 5: Exemplary synthesis of 3-[4-fluoro-1-oxo-5-(4-{[(1r, 4r)-4-[(3-methyl-4-{[2-(3-{3-[(4-methyl-4H-1,2,4-triazol-3-yl)methyl]oxetan-3-yl}phenyl)-3-oxo-7-(trifluoromethyl)-2,3-dihydro-1H-isoindol-5-yl]methyl}piperazin-1-yl)methyl]cyclohexyl]oxy}piperidin-1-yl)-2,3-dihydro-1H-isoindol-2-yl]piperidine-2,6-dione

### Step 1: Preparation of tert-butyl 3-methyl-4-[[2-[3-[3-[(4-methyl-1,2,4-triazol-3-yl)methyl]oxetan-3-yl]phenyl]-3-oxo-7-(trifluoromethyl)isoindolin-5-yl]methyl]piperazine-1-carboxylate

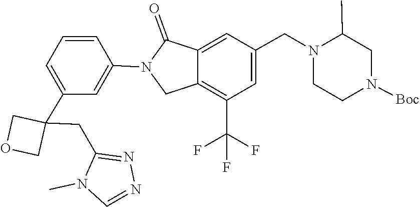 [SMILES: CC1CN(C(=O)OC(C)(C)C)CCN1Cc1cc2c(c(C(F)(F)F)c1)CN(c1cccc(C3(Cc4nncn4C)COC3)c1)C2=O]

To a solution of 2-[3-[3-[(4-methyl-1,2,4-triazol-3-yl)methyl]oxetan-3-yl]phenyl]-3-oxo-7-(trifluoromethyl)isoindoline-5-carbaldehyde (60 mg, 0.1 mmol), tert-butyl 3-methylpiperazine-1-carboxylate (53 mg, 0.3 mmol) and tetraisopropoxytitanium (37 mg, 0.1 mmol) in methanol (2 mL) was added sodium cyanoborohydride (17 mg, 0.3 mmol). The mixture was stirred at 25° C. for 10 h, then filtered and concentrated. The residue was purified by column chromatography (petroleum ether/ethyl acetate=4/1 to 1/1 to dichloromethane/methanol=50/1 to 20/1) to give tert-butyl 3-methyl-4-[[2-[3-[3-[(4-methyl-1,2,4-triazol-3-yl)methyl]oxetan-3-yl]phenyl]-3-oxo-7-(trifluoromethyl)isoindolin-5-yl]methyl]piperazine-1-carboxylate (52 mg, 62%) as a white solid. MS (ESI) m/z: 641.4 [M+H]⁺.

### Step 2: Preparation of 6-[(2-methylpiperazin-1-yl)methyl]-2-[3-[3-[(4-methyl-1,2,4-triazol-3-yl)methyl]oxetan-3-yl]phenyl]-4-(trifluoromethyl)isoindolin-1-one

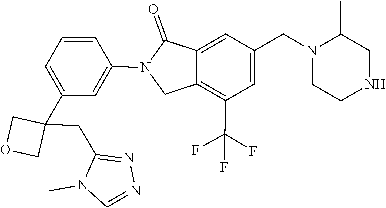 [SMILES: CC1CNCCN1Cc1cc2c(c(C(F)(F)F)c1)CN(c1cccc(C3(Cc4nncn4C)COC3)c1)C2=O]

To a solution of tert-butyl 3-methyl-4-[[2-[3-[3-[(4-methyl-1,2,4-triazol-3-yl)methyl]oxetan-3-yl]phenyl]-3-oxo-7-(trifluoromethyl)isoindolin-5-yl]methyl]piperazine-1-carboxylate (52 mg, 0.08 mmol) in dichloromethane (2 mL) was added trifluoroacetic acid (1.0 mL, 13 mmol). The mixture was stirred at 25° C. for 0.5 h, then concentrated under reduced pressure to give 6-[(2-methylpiperazin-1-yl)methyl]-2-[3-[3-[(4-methyl-1,2,4-triazol-3-yl)methyl]oxetan-3-yl]phenyl]-4-(trifluoromethyl)isoindolin-1-one trifluoroacetate (52 mg, 98%) as a yellow oil. MS (ESI) m/z: 541.4 [M+H]⁺.

### Step 3: Preparation of 3-[4-fluoro-5-[4-[4-[[3-methyl-4-[[2-[3-[3-[(4-methyl-1,2,4-triazol-3-yl)methyl]oxetan-3-yl]phenyl]-3-oxo-7-(trifluoromethyl)isoindolin-5-yl]methyl]piperazin-1-yl]methyl]cyclohexoxy]-1-piperidyl]-1-oxo-isoindolin-2-yl]piperidine-2,6-dione

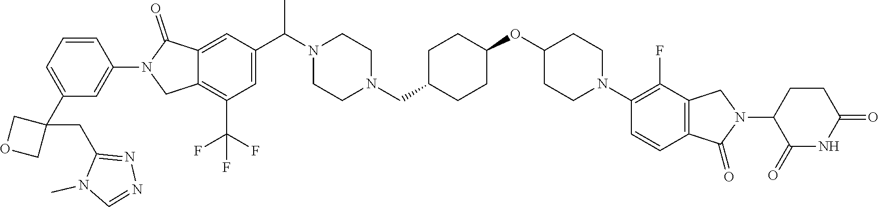 [SMILES: CC(c1cc2c(c(C(F)(F)F)c1)CN(c1cccc(C3(Cc4nncn4C)COC3)c1)C2=O)N1CCN(C[C@H]2CC[C@H](OC3CCN(c4ccc5c(c4F)CN(C4CCC(=O)NC4=O)C5=O)CC3)CC2)CC1]

To a solution of 6-[(2-methylpiperazin-1-yl)methyl]-2-[3-[3-[(4-methyl-1,2,4-triazol-3-yl)methyl]oxetan-3-yl]phenyl]-4-(trifluoromethyl)isoindolin-1-one trifluoroacetate (52 mg, 0.08 mmol) in N,N-dimethylformamide (1 mL) was added nitromethyl morpholine (19 mg, 0.2 mmol), 4-[[1-[2-(2,6-dioxo-3-piperidyl)-4-fluoro-1-oxo-isoindolin-5-yl]-4-piperidyl]oxy]cyclohexanecarbaldehyde (90 mg, 0.2 mmol) and sodium triacetoxyborohydride (61 mg, 0.3 mmol) at 0° C. The mixture was stirred at 25° C. for 10 h, then filtered and concentrated. The residue was purified by preparative HPLC (column: Phenomenex Luna C18 150×25 mm×10 um; mobile phase: [water(FA)-ACN]; B %: 18%-48%, 11 min) to give 3-[4-fluoro-5-[4-[4-[[3-methyl-4-[[2-[3-[3-[(4-methyl-1,2,4-triazol-3-yl)methyl]oxetan-3-yl]phenyl]-3-oxo-7-(trifluoromethyl)isoindolin-5-yl]methyl]piperazin-1-yl]methyl]cyclohexoxy]-1-piperidyl]-1-oxo-isoindolin-2-yl]piperidine-2,6-dione formate (28.8 mg, 29%) as a white solid. MS (ESI) m/z: 997.3 [M+H]⁺; ¹H NMR (400 MHz, DMSO-d₆) δ 10.85-11.14 (m, 1H), 8.20 (s, 1H), 8.17 (s, 1H), 7.91-8.01 (m, 2H), 7.89 (br d, J=8.8 Hz, 1H), 7.47 (d, J=8.0 Hz, 1H), 7.31-7.43 (m, 2H), 7.12-7.25 (m, 1H), 6.77 (br d, J=8.0 Hz, 1H), 5.04-5.14 (m, 3H), 4.97 (br d, J=6.0 Hz, 2H), 4.89 (d, J=6.0 Hz, 2H), 4.48 (br d, J=16.8 Hz, 1H), 4.31 (br d, J=16.8 Hz, 1H), 4.08-4.14 (m, 1H), 3.57-3.66 (m, 2H), 3.52 (s, 3H), 3.43-3.47 (m, 2H), 2.96 (br d, J=1.2 Hz, 2H), 2.90 (s, 3H), 2.57-2.64 (m, 2H), 2.38-2.47 (m, 4H), 2.16-2.22 (m, 1H), 1.97-2.07 (m, 4H), 1.88-1.96 (m, 5H), 1.76 (br dd, J=8.0, 2.4 Hz, 2H), 1.52-1.62 (m, 2H), 1.38-1.46 (m, 1H), 1.03-1.19 (m, 6H), 0.81-0.93 ppm (m, 2H).

### Example 6: Exemplary synthesis of 3-[4-fluoro-1-oxo-5-(4-{[(1r, 4r)-4-[(2-{[2-(3-{3-[(4-methyl-4H-1,2,4-triazol-3-yl)methyl]oxetan-3-yl}phenyl)-3-oxo-7-(trifluoromethyl)-2,3-dihydro-1H-isoindol-5-yl]methyl}-2,7-diazaspiro[3.5]nonan-7-yl)methyl]cyclohexyl]oxy}piperidin-1-yl)-2,3-dihydro-1H-isoindol-2-yl]piperidine-2,6-dione

### Step 1: Preparation of tert-butyl 2-[[2-[3-[3-[(4-methyl-1,2,4-triazol-3-yl)methyl]oxetan-3-yl]phenyl]-3-oxo-7-(trifluoromethyl)isoindolin-5-yl]methyl]-2,7-diazaspiro[3.5]nonane-7-carboxylate

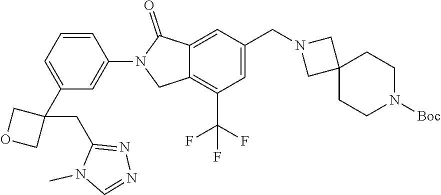 [SMILES: Cn1cnnc1CC1(c2cccc(N3Cc4c(cc(CN5CC6(CCN(C(=O)OC(C)(C)C)CC6)C5)cc4C(F)(F)F)C3=O)c2)COC1]

To a solution of 2-[3-[3-[(4-methyl-1,2,4-triazol-3-yl)methyl]oxetan-3-yl]phenyl]-3-oxo-7-(trifluoromethyl)isoindoline-5-carbaldehyde (100 mg, 0.2 mmol) in dichloromethane (2 mL) was added tert-butyl 2,7-diazaspiro[3.5]nonane-7-carboxylate (74 mg, 0.3 mmol) and stirred at 25° C. for 10 h. Sodium triacetoxyborohydride (186 mg, 0.9 mmol) was added and the mixture was stirred at 25° C. for 0.5 h, then concentrated under reduced pressure. The residue was purified by preparative thin layer chromatography (dichloromethane/methanol=10/1) to afford tert-butyl 2-[[2-[3-[3-[(4-methyl-1,2,4-triazol-3-yl)methyl]oxetan-3-yl]phenyl]-3-oxo-7-(trifluoromethyl)isoindolin-5-yl]methyl]-2,7-diazaspiro[3.5]nonane-7-carboxylate (120 mg, 82%) as white solid. MS (ESI) m/z: 667.1 [M+H]⁺.

### Step 2: Preparation of 6-(2,7-diazaspiro[3.5]nonan-2-ylmethyl)-2-[3-[3-[(4-methyl-1,2,4-triazol-3-yl)methyl]oxetan-3-yl]phenyl]-4-(trifluoromethyl)isoindolin-1-one

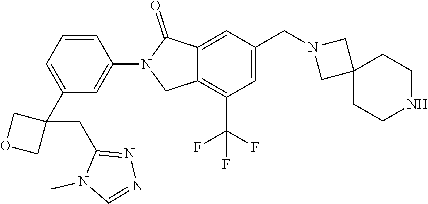 [SMILES: Cn1cnnc1CC1(c2cccc(N3Cc4c(cc(CN5CC6(CCNCC6)C5)cc4C(F)(F)F)C3=O)c2)COC1]

To a solution of tert-butyl 2-[[2-[3-[3-[(4-methyl-1,2,4-triazol-3-yl)methyl]oxetan-3-yl]phenyl]-3-oxo-7-(trifluoromethyl)isoindolin-5-yl]methyl]-2,7-diazaspiro[3.5]nonane-7-carboxylate (60 mg, 0.09 mmol) in dichloromethane (2 mL) was added trifluoroacetic acid (20 mg, 0.2 mmol). The mixture was stirred at 25° C. for 0.5 h, then concentrated under reduced pressure to afford 6-(2,7-diazaspiro[3.5]nonan-2-ylmethyl)-2-[3-[3-[(4-methyl-1,2,4-triazol-3-yl)methyl]oxetan-3-yl]phenyl]-4-(trifluoromethyl)isoindolin-1-one (60 mg, crude) as a yellow oil. MS (ESI) m/z: 567.4 [M+H]⁺.

### Step 3: Preparation of 3-[4-fluoro-5-[4-[4-[[2-[[2-[3-[3-[(4-methyl-1,2,4-triazol-3-yl)methyl]oxetan-3-yl]phenyl]-3-oxo-7-(trifluoromethyl)isoindolin-5-yl]methyl]-2,7-diazaspiro[3.5]nonan-7-yl]methyl]cyclohexoxy]-1-piperidyl]-1-oxo-isoindolin-2-yl]piperidine-2,6-dione

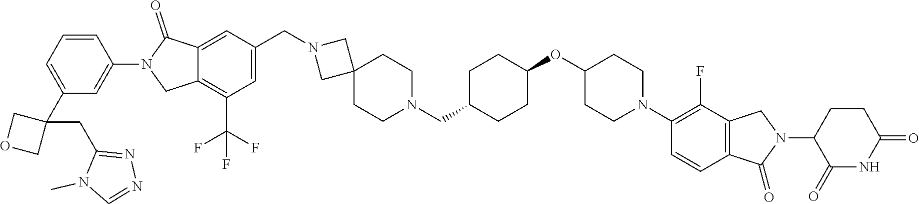 [SMILES: Cn1cnnc1CC1(c2cccc(N3Cc4c(cc(CN5CC6(CCN(C[C@H]7CC[C@H](OC8CCN(c9ccc%10c(c9F)CN(C9CCC(=O)NC9=O)C%10=O)CC8)CC7)CC6)C5)cc4C(F)(F)F)C3=O)c2)COC1]

To a solution of 6-(2,7-diazaspiro[3.5]nonan-2-ylmethyl)-2-[3-[3-[(4-methyl-1,2,4-triazol-3-yl)methyl]oxetan-3-yl]phenyl]-4-(trifluoromethyl)isoindolin-1-one (60 mg, 0.1 mmol) in N,N-dimethylformamide (2 mL) was added 4-[[1-[2-(2,6-dioxo-3-piperidyl)-4-fluoro-1-oxo-isoindolin-5-yl]-4-piperidyl]oxy]cyclohexanecarbaldehyde (100 mg, 0.2 mmol) and 4-methylmorpholine (32 mg, 0.3 mmol) at 0° C. The mixture was stirred at 0° C. for 0.5 h and then sodium triacetoxyborohydride (67 mg, 0.3 mmol) was added. The mixture was stirred at 25° C. for 10 h. Water (10 mL) was added and the mixture was extracted with ethyl acetate (3×10 mL). The organic layers were concentrated under reduced pressure. The residue was purified by preparative HPLC (column: Phenomenex Luna C18 150*25 mm*10 um; mobile phase: [formic acid in water-acetonitrile]; B %: 2%-32%, 10 min) to afford 3-[4-fluoro-5-[4-[4-[[2-[[2-[3-[3-[(4-methyl-1,2,4-triazol-3-yl)methyl]oxetan-3-yl]phenyl]-3-oxo-7-(trifluoromethyl)isoindolin-5-yl]methyl]-2,7-diazaspiro[3.5]nonan-7-yl]methyl]cyclohexoxy]-1-piperidyl]-1-oxo-isoindolin-2-yl]piperidine-2,6-dione formate (60.7 mg, 49%) as a white solid. MS (ESI) m/z: 1022.3 [M+H]⁺; ¹H NMR (400 MHz, DMSO-d₆) δ 10.99 (s, 1H), 8.20-8.17 (m, 3H), 7.94 (s, 1H), 7.91-7.85 (m, 2H), 7.45 (d, J=8.0 Hz, 1H), 7.40 (s, 1H), 7.35 (t, J=8.0 Hz, 1H), 7.16 (t, J=8.0 Hz, 1H), 6.76 (d, J=8.0 Hz, 1H), 5.11-5.04 (m, 3H), 4.96 (d, J=6.0 Hz, 2H), 4.88 (d, J=6.0 Hz, 2H), 4.47 (d, J=17.2 Hz, 1H), 4.30 (d, J=16.8 Hz, 1H), 3.82 (s, 2H), 3.63-3.57 (m, 1H), 3.51 (s, 2H), 3.01 (s, 4H), 2.96-2.88 (m, 6H), 2.61-2.56 (m, 1H), 2.54 (s, 1H), 2.41 (d, J=8.8 Hz, 1H), 2.32 (dd, J=2.4, 6.4 Hz, 3H), 2.12-2.06 (m, 2H), 2.01-1.85 (m, 6H), 1.81-1.63 (m, 7H), 1.61-1.37 (m, 4H), 1.13 (q, J=11.2 Hz, 2H), 0.91-0.79 (m, 2H).

### Example 7: Exemplary synthesis of 3-[4-fluoro-1-oxo-5-(4-{[(1r, 4r)-4-({2-[2-(3-{3-[(4-methyl-4H-1,2,4-triazol-3-yl)methyl]oxetan-3-yl}phenyl)-3-oxo-7-(trifluoromethyl)-2,3-dihydro-1H-isoindol-5-yl]-2,7-diazaspiro [3.5]nonan-7-yl}methyl)cyclohexyl]oxy}piperidin-1-yl)-2,3-dihydro-1H-isoindol-2-yl]piperidine-2,6-dione

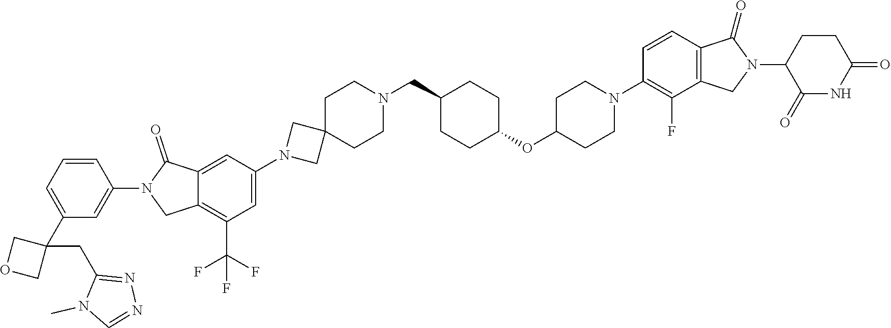 [SMILES: Cn1cnnc1CC1(c2cccc(N3Cc4c(cc(N5CC6(CCN(C[C@H]7CC[C@H](OC8CCN(c9ccc%10c(c9F)CN(C9CCC(=O)NC9=O)C%10=O)CC8)CC7)CC6)C5)cc4C(F)(F)F)C3=O)c2)COC1]

To a stirred solution of 6-(2,7-diazaspiro[3.5]nonan-2-yl)-2-[3-[3-[(4-methyl-1,2,4-triazol-3-yl)methyl]oxetan-3-yl]phenyl]-4-(trifluoromethyl)isoindolin-1-one trifluoroacetate (55 mg, 0.01 mmol) in N,N-dimethylformamide (2 mL) was added nitromethyl morpholine (25 mg, 0.3 mmol) and 4-[[1-[2-(2,6-dioxo-3-piperidyl)-4-fluoro-1-oxo-isoindolin-5-yl]-4-piperidyl]oxy]cyclohexanecarbaldehyde (39 mg, 0.01 mmol) at 0° C., followed by sodium triacetoxyborohydride (53 mg, 0.3 mmol). The mixture was then stirred at 25° C. for 10 h. The reaction mixture was purified by preparative HPLC (column: Phenomenex Luna C18 150*25 mm*10 um; mobile phase: [formic acid in water-acetonitrile]; B %: 14%-44%, 10 min) to give 3-[4-fluoro-5-[4-[4-[[2-[2-[3-[3-[(4-methyl-1,2,4-triazol-3-yl)methyl]oxetan-3-yl]phenyl]-3-oxo-7-(trifluoromethyl)isoindolin-5-yl]-2,7-diazaspiro[3.5]nonan-7-yl]methyl]cyclohexoxy]-1-piperidyl]-1-oxo-isoindolin-2-yl]piperidine-2,6-dione di-formate (20.4 mg, 21%) as a white solid. MS (ESI) m/z: 1008.5 [M+H]⁺; ¹H NMR (400 MHz, DMSO-d₆) δ 10.99 (s, 1H), 8.19 (s, 3H), 7.88 (d, J=8.0 Hz, 1H), 7.47 (d, J=8.0 Hz, 1H), 7.40-7.28 (m, 2H), 7.17 (t, J=8.0 Hz, 1H), 6.93 (s, 2H), 6.75 (d, J=8.0 Hz, 1H), 5.08 (dd, J=4.4, 12.8 Hz, 1H), 5.03-4.92 (m, 4H), 4.89 (d, J=6.0 Hz, 2H), 4.54-4.42 (m, 1H), 4.37-4.26 (m, 1H), 3.69 (s, 4H), 3.65-3.58 (m, 2H), 3.51 (s, 3H), 2.95 (dd, J=1.6, 6.0 Hz, 2H), 2.90 (s, 4H), 2.64-2.56 (m, 2H), 2.40-2.20 (m, 5H), 2.10-2.05 (m, 2H), 1.99-1.88 (m, 5H), 1.76 (s, 6H), 1.63-1.52 (m, 2H), 1.50-1.38 (m, 1H), 1.23-1.09 (m, 2H), 0.97-0.79 (m, 2H).

### Example 8: Exemplary synthesis of 3-{4-methoxy-5-[4-({2-[2-(3-{3-[(4-methyl-4H-1,2,4-triazol-3-yl)methyl]oxetan-3-yl}phenyl)-3-oxo-7-(trifluoromethyl)-2,3-dihydro-1H-isoindol-5-yl]-2,7-diazaspiro [3.5]nonan-7-yl}methyl)piperidin-1-yl]-1-oxo-2,3-dihydro-1H-isoindol-2-yl}piperidine-2,6-dione

### Step 1: Preparation of 2,6-bis(benzyloxy)pyridin-3-amine

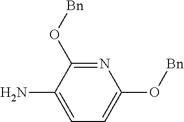 [SMILES: Nc1ccc(OCc2ccccc2)nc1OCc1ccccc1]

To a solution of phenylmethanol (16.0 mL, 153 mmol) in tetrahydrofuran (200 mL) was added potassium tert-butoxide (17.21 g, 153 mmol) portion-wise. The mixture was stirred at 25° C. for 2 h. Then 2,6-dichloropyridin-3-amine (10 g, 61 mmol) was added and stirred at 75° C. for 24 h. The mixture was cooled to 25° C. and diluted with ethyl acetate (600 mL). The organic layer was washed with water (100 mL×3). The water layer was further extracted with ethyl acetate (100 mL×2). The combined organic layer was dried over anhydrous sodium sulfate, filtered, and concentrated. The residue was purified by silica gel column chromatography (petroleum ether/ethyl acetate=50/1 to 10:1), and the crude product was further purified by preparative HPLC (column: Welch Ultimate XB-Diol 250*50*10 um; mobile phase: [heptane-ethanol (0.1% NH₃H₂O)]; B %: 1%-10%, 15 min) to afford 2,6-bis(benzyloxy)pyridin-3-amine (3.1 g, 16%) as a dark orange oil. MS (ESI) m/z: 307.4 [M+H]⁺; ¹H NMR (400 MHz, CDCl₃) δ 7.51-7.30 (m, 10H), 6.99 (d, J=8.0 Hz, 1H), 6.28 (d, J=8.0 Hz, 1H), 5.39 (s, 2H), 5.28 (s, 2H), 3.27-2.56 (m, 2H).

### Step 2: Preparation of methyl 4-bromo-3-methoxy-2-methyl-benzoate

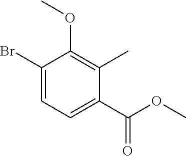

To a solution of methyl 4-bromo-3-hydroxy-2-methyl-benzoate (5.0 g, 20 mmol) in acetonitrile (50 mL) was added potassium carbonate (5.64 g, 41 mmol) and iodomethane (2.5 mL, 41 mmol). The mixture was stirred at 60° C. for 12 h, then filtered and concentrated. The residue was purified by flash silica gel chromatography (0-15% ethyl acetate/petroleum ether) to afford methyl 4-bromo-3-methoxy-2-methyl-benzoate (5.2 g, 98%) as a white solid. MS (ESI) m/z: 259.0 [M+H]⁺; ¹H NMR (400 MHz, CDCl₃) δ 7.54-7.50 (m, 1H), 7.47-7.43 (m, 1H), 3.89 (s, 3H), 3.81 (s, 3H), 2.56 (s, 3H).

### Step 3: Preparation of methyl 4-bromo-2-(bromomethyl)-3-methoxy-benzoate

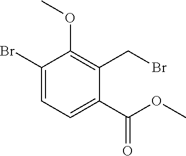

To a solution of methyl 4-bromo-3-methoxy-2-methyl-benzoate (5.2 g, 20 mmol) in carbon tetrachloride (50 mL) was added 1-bromopyrrolidine-2,5-dione (3.57 g, 20 mmol) and azobisisobutyronitrile (330 mg, 2 mmol). The mixture was stirred at 80° C. for 1 h, then filtered and concentrated. The residue was purified by flash silica gel chromatography (0-20% ethyl acetate/petroleum ether) to afford methyl 4-bromo-2-(bromomethyl)-3-methoxy-benzoate (6.5 g, 96%) as a colorless oil. ¹H NMR (400 MHz, DMSO-d₆) δ 7.79 (d, J=8.4 Hz, 1H), 7.59 (d, J=8.4 Hz, 1H), 5.01 (s, 2H), 3.92 (s, 3H), 3.87 (s, 3H).

### Step 4: Preparation of 5-bromo-2-(2,6-dibenzyloxy-3-pyridyl)-4-methoxy-isoindolin-1-one

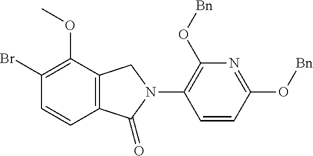 [SMILES: COc1c(Br)ccc2c1CN(c1ccc(OCc3ccccc3)nc1OCc1ccccc1)C2=O]

To a solution of methyl 4-bromo-2-(bromomethyl)-3-methoxy-benzoate (1 g, 3 mmol) and N,N-diisopropylethylamine (1.6 mL, 9 mmol,) 2,6-dibenzyloxypyridin-3-amine (1 g, 3 mmol) in N,N-dimethylacetamide (10 mL) was added 2,6-dibenzyloxypyridin-3-amine (1.00 g, 3 mmol) at 0° C. The mixture was stirred at 120° C. for 12 h, then diluted with water (100 mL) and extracted with ethyl acetate (40 mL×3). The organic phase was combined and washed with saturated brine (40 mL×3), dried over anhydrous sodium sulfate, filtered, and concentrated. The residue was purified by flash silica gel chromatography (0-100% ethyl acetate/petroleum ether) to afford 5-bromo-2-(2,6-dibenzyloxy-3-pyridyl)-4-methoxy-isoindolin-1-one (1 g, 64%) as a yellow solid. MS (ESI) m/z: 533.2 [M+H]⁺; ¹H NMR (400 MHz, CDCl₃) δ 7.70 (t, J=9.6 Hz, 2H), 7.53 (d, J=8.4 Hz, 1H), 7.43-7.31 (m, 10H), 6.51 (d, J=8.0 Hz, 1H), 5.38 (d, J=14.0 Hz, 4H), 4.85 (s, 2H), 3.90 (s, 3H).

### Step 5: Preparation of 2-(2,6-dibenzyloxy-3-pyridyl)-5-[4-(dimethoxymethyl)-1-piperidyl]-4-methoxy-isoindolin-1-one

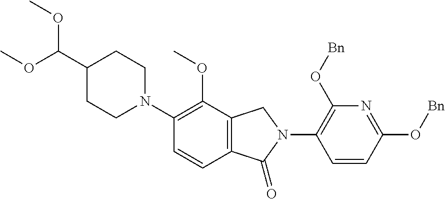 [SMILES: COc1c(N2CCC(C(OC)OC)CC2)ccc2c1CN(c1ccc(OCc3ccccc3)nc1OCc1ccccc1)C2=O]

A mixture of 5-bromo-2-(2,6-dibenzyloxy-3-pyridyl)-4-methoxy-isoindolin-1-one (1 g, 1.88 mmol, 1 eq), 4-(dimethoxymethyl)piperidine (360 mg, 2.3 mmol), cesium carbonate (1.23 g, 3.8 mmol) and chloro(2-dicyclohexylphosphino-2′,4′,6′-triisopropyl-1,1′-biphenyl)[2-(2′-amino-1,1′-biphenyl)]palladium(II) (148 mg, 0.2 mmol) in dioxane (10 mL) was degassed and purged with nitrogen three times, then the mixture was stirred at 100° C. for 2 h under nitrogen atmosphere. The reaction mixture was filtered, and the filtrate was concentrated. The residue was purified by flash silica gel chromatography (0-50% ethyl acetate/petroleum ether) to afford 2-(2,6-dibenzyloxy-3-pyridyl)-5-[4-(dimethoxymethyl)-1-piperidyl]-4-methoxy-isoindolin-1-one (350 mg, 31%) as a yellow solid. MS (ESI) m/z: 610.5 [M+H]⁺.

### Step 6: Preparation of 3-[5-[4-(dimethoxymethyl)-1-piperidyl]-4-methoxy-1-oxo-isoindolin-2-yl]piperidine-2,6-dione

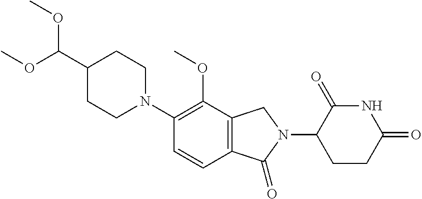 [SMILES: COc1c(N2CCC(C(OC)OC)CC2)ccc2c1CN(C1CCC(=O)NC1=O)C2=O]

To a solution of 2-(2,6-dibenzyloxy-3-pyridyl)-5-[4-(dimethoxymethyl)-1-piperidyl]-4-methoxy-isoindolin-1-one (350 mg, 0.57 mmol) in ethyl acetate (5 mL) was added 10% palladium on activated carbon (50 mg). The suspension was degassed and purged with hydrogen three times. The mixture was stirred under hydrogen atmosphere (50 Psi) at 25° C. for 12 h, then filtered and concentrated. The residue was purified by preparative HPLC (column: Phenomenex Luna C18 150×25 mm×10 um; mobile phase: [water(FA)-ACN]; B %: 13%-43%, 10 min) to afford 3-[5-[4-(dimethoxymethyl)-1-piperidyl]-4-methoxy-1-oxo-isoindolin-2-yl]piperidine-2,6-dione (50 mg, 20%) as a white solid. MS (ESI) m/z: 432.3 [M+H]⁺; ¹H NMR (400 MHz, CDCl₃) δ 7.91 (s, 1H), 7.55 (d, J=8.0 Hz, 1H), 7.04 (d, J=8.4 Hz, 1H), 5.20 (d, J=8.4 Hz, 1H), 4.47-4.40 (m, 1H), 4.32-4.27 (m, 1H), 4.13 (d, J=7.2 Hz, 1H), 3.91 (s, 3H), 3.73-3.60 (m, 3H), 3.40 (s, 6H), 2.97-2.89 (m, 1H), 2.88-2.82 (m, 1H), 2.69-2.63 (m, 2H), 2.42-2.31 (m, 1H), 2.25-2.17 (m, 1H), 1.89 (d, J=12.4 Hz, 2H), 1.78 (dt, J=4.0, 7.6 Hz, 1H), 1.53-1.50 (m, 1H).

### Step 7: Preparation of 1-[2-(2,6-dioxo-3-piperidyl)-4-methoxy-1-oxo-isoindolin-5-yl]piperidine-4-carbaldehyde

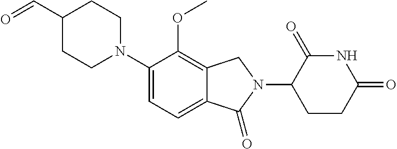 [SMILES: COc1c(N2CCC(C=O)CC2)ccc2c1CN(C1CCC(=O)NC1=O)C2=O]

To a solution of 3-[5-[4-(dimethoxymethyl)-1-piperidyl]-4-methoxy-1-oxo-isoindolin-2-yl]piperidine-2,6-dione (50 mg, 0.1 mmol) in dichloromethane (1 mL) was added trifluoroacetic acid (0.5 mL, 7 mmol). The mixture was stirred at 25° C. for 0.5 h, then concentrated under reduced pressure to afford 1-[2-(2,6-dioxo-3-piperidyl)-4-methoxy-1-oxo-isoindolin-5-yl]piperidine-4-carbaldehyde (44 mg, 99%) as a yellow oil. MS (ESI) m/z: 386.2 [M+H]⁺.

### Step 8: Preparation of 3-[4-methoxy-5-[4-[[2-[2-[3-[3-[(4-methyl-1,2,4-triazol-3-yl)methyl]oxetan-3-yl]phenyl]-3-oxo-7-(trifluoromethyl)isoindolin-5-yl]-2,7-diazaspiro[3.5]nonan-7-yl]methyl]-1-piperidyl]-1-oxo-isoindolin-2-yl]piperidine-2,6-dione

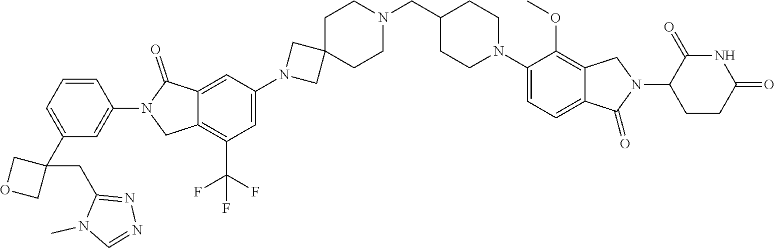 [SMILES: COc1c(N2CCC(CN3CCC4(CC3)CN(c3cc5c(c(C(F)(F)F)c3)CN(c3cccc(C6(Cc7nncn7C)COC6)c3)C5=O)C4)CC2)ccc2c1CN(C1CCC(=O)NC1=O)C2=O]

To a solution of 1-[2-(2,6-dioxo-3-piperidyl)-4-methoxy-1-oxo-isoindolin-5-yl]piperidine-4-carbaldehyde (97 mg, 0.3 mmol) and 6-(2,7-diazaspiro[3.5]nonan-2-yl)-2-[3-[3-[(4-methyl-1,2,4-triazol-3-yl)methyl]oxetan-3-yl]phenyl]-4-(trifluoromethyl)isoindolin-1-one (77 mg, 0.1 mmol) in N,N-dimethylformamide (2 mL) was added trimethylamine (56 mg, 0.6 mmol). The mixture was stirred at 25° C. for 1 h and then added sodium triacetoxyborohydride (118 mg, 0.6 mmol). The mixture was stirred at 25° C. for 10 h. Water (10 mL) was added, and the mixture was extracted with ethyl acetate (10 mL×3). The organic layers were concentrated under reduced pressure. The residue was purified by preparative HPLC (column: Phenomenex Luna C18 150×25 mm×10 um; mobile phase: [water(formic acid)-acetonitrile]; B %: 10%-40%, 10 min) to afford 3-[4-methoxy-5-[4-[[2-[2-[3-[3-[(4-methyl-1,2,4-triazol-3-yl)methyl]oxetan-3-yl]phenyl]-3-oxo-7-(trifluoromethyl)isoindolin-5-yl]-2,7-diazaspiro[3.5]nonan-7-yl]methyl]-1-piperidyl]-1-oxo-isoindolin-2-yl]piperidine-2,6-dione (41 mg, 31%) as a white solid. MS (ESI) m/z: 922.6 [M+H]⁺; ¹H NMR (400 MHz, DMSO-d₆) δ 10.97 (s, 1H), 8.19 (s, 1H), 7.87 (d, J=8.4 Hz, 1H), 7.41-7.31 (m, 3H), 7.09 (d, J=8.0 Hz, 1H), 6.93 (d, J=8.8 Hz, 2H), 6.76 (d, J=7.6 Hz, 1H), 5.06 (dd, J=5.2, 13.2 Hz, 1H), 5.00-4.94 (m, 4H), 4.88 (d, J=6.0 Hz, 2H), 4.45 (d, J=17.2 Hz, 1H), 4.27 (d, J=16.8 Hz, 1H), 3.87 (s, 3H), 3.78-3.75 (m, 4H), 3.60-3.50 (m, 4H), 2.95-2.86 (m, 5H), 2.69 (t, J=10.4 Hz, 3H), 2.63-2.58 (m, 1H), 2.59-2.53 (m, 2H), 2.51-2.51 (m, 2H), 2.43 (dd, J=4.4, 13.6 Hz, 1H), 2.02-1.82 (m, 8H), 1.45-1.31 (m, 2H).

### Example 9: Exemplary synthesis of 3-[4-fluoro-1-oxo-5-(4-{[(1r, 4r)-4-[(4-{1-[2-(3-{3-[(4-methyl-4H-1,2,4-triazol-3-yl)methyl]oxetan-3-yl}phenyl)-3-oxo-7-(trifluoromethyl)-2,3-dihydro-1H-isoindol-5-yl]ethyl}piperazin-1-yl)methyl]cyclohexyl]oxy}piperidin-1-yl)-2,3-dihydro-1H-isoindol-2-yl]piperidine-2,6-dione

### Step 1: Preparation of 6-(1-ethoxyvinyl)-2-[3-[3-[(4-methyl-1,2,4-triazol-3-yl)methyl]oxetan-3-yl]phenyl]-4-(trifluoromethyl)isoindolin-1-one

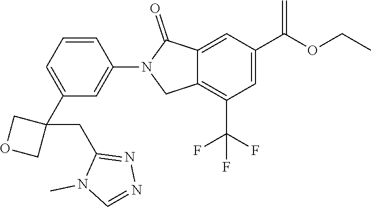 [SMILES: C=C(OCC)c1cc2c(c(C(F)(F)F)c1)CN(c1cccc(C3(Cc4nncn4C)COC3)c1)C2=O]

To a solution of 6-bromo-2-[3-[3-[(4-methyl-1,2,4-triazol-3-yl)methyl]oxetan-3-yl]phenyl]-4-(trifluoromethyl)isoindolin-1-one (500 mg, 1.0 mmol) in dioxane (5 mL) was added palladium triphenylphosphane (25 mg, 0.07 mmol) and tributyl(1-ethoxyvinyl)stannane (616 mg, 1.7 mmol). The mixture was stirred at 80° C. for 2 h under nitrogen. The reaction was quenched with saturated potassium fluoride solution (10 mL) and extracted with ethyl acetate (10 mL). The organic layer was washed with brine (10 mL), dried over anhydrous sodium sulfate, and concentrated under reduced pressure to afford 6-(1-ethoxyvinyl)-2-[3-[3-[(4-methyl-1,2,4-triazol-3-yl)methyl]oxetan-3-yl]phenyl]-4-(trifluoromethyl)isoindolin-1-one (500 mg, crude) as a yellow oil. MS (ESI) m/z: 499.2 [M+H]⁺.

### Step 2: Preparation of 6-acetyl-2-[3-[3-[(4-methyl-1,2,4-triazol-3-yl)methyl]oxetan-3-yl]phenyl]-4-(trifluoromethyl)isoindolin-1-one

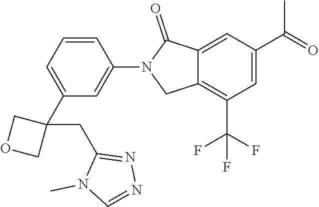 [SMILES: CC(=O)c1cc2c(c(C(F)(F)F)c1)CN(c1cccc(C3(Cc4nncn4C)COC3)c1)C2=O]

To a solution of 6-(1-ethoxyvinyl)-2-[3-[3-[(4-methyl-1,2,4-triazol-3-yl)methyl]oxetan-3-yl]phenyl]-4-(trifluoromethyl)isoindolin-1-one (500 mg, 1 mmol) in tetrahydrofuran (5 mL) was added 2M hydrochloric acid (1.5 mL). The mixture was stirred at 25° C. for 2 h, then quenched with saturated sodium bicarbonate solution (10 mL) and extracted with ethyl acetate (10 mL). The organic layer was washed with brine (10 mL), dried over anhydrous sodium sulfate, and concentrated under reduced pressure. The residue was purified by silica gel column chromatography (dichloromethane/methanol=100/1 to 10/1) to afford 6-acetyl-2-[3-[3-[(4-methyl-1,2,4-triazol-3-yl)methyl]oxetan-3-yl]phenyl]-4-(trifluoromethyl)isoindolin-1-one (450 mg, 95%) as a yellow solid. MS (ESI) m/z: 471.1 [M+H]⁺.

### Step 3: Preparation of tert-butyl 4-[1-[2-[3-[3-[(4-methyl-1,2,4-triazol-3-yl)methyl]oxetan-3-yl]phenyl]-3-oxo-7-(trifluoromethyl)isoindolin-5-yl]ethyl]piperazine-1-carboxylate

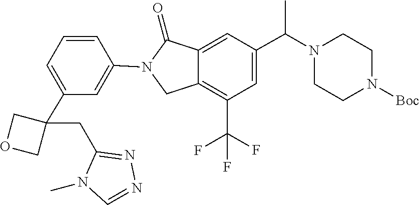 [SMILES: CC(c1cc2c(c(C(F)(F)F)c1)CN(c1cccc(C3(Cc4nncn4C)COC3)c1)C2=O)N1CCN(C(=O)OC(C)(C)C)CC1]

To a solution of 6-acetyl-2-[3-[3-[(4-methyl-1,2,4-triazol-3-yl)methyl]oxetan-3-yl]phenyl]-4-(trifluoromethyl)isoindolin-1-one (200 mg, 0.4 mmol) and tert-butyl piperazine-1-carboxylate (317 mg, 1.7 mmol) in dichloromethane (2 mL) was added chloro(triisopropoxy)titanium (1 M, 0.43 mL). The mixture was stirred at 25° C. for 10 h, then sodium triacetoxyborohydride (180 mg, 0.9 mmol) was added and the mixture was stirred for 0.5 h. The reaction was concentrated and the residue was purified by preparative thin layer chromatography (dichloromethane/methanol=10/1) to afford tert-butyl 4-[1-[2-[3-[3-[(4-methyl-1,2,4-triazol-3-yl)methyl]oxetan-3-yl]phenyl]-3-oxo-7-(trifluoromethyl)isoindolin-5-yl]ethyl]piperazine-1-carboxylate (100 mg, 37%) as a white solid. MS (ESI) m/z: 641.3 [M+H]⁺.

### Step 4: Preparation of 2-[3-[3-[(4-methyl-1,2,4-triazol-3-yl)methyl]oxetan-3-yl]phenyl]-6-(1-piperazin-1-ylethyl)-4-(trifluoromethyl)isoindolin-1-one

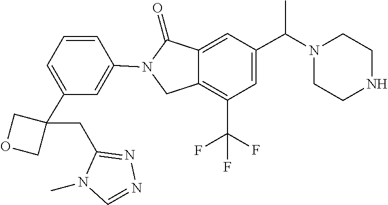 [SMILES: CC(c1cc2c(c(C(F)(F)F)c1)CN(c1cccc(C3(Cc4nncn4C)COC3)c1)C2=O)N1CCNCC1]

To a solution of tert-butyl 4-[1-[2-[3-[3-[(4-methyl-1,2,4-triazol-3-yl)methyl]oxetan-3-yl]phenyl]-3-oxo-7-(trifluoromethyl)isoindolin-5-yl]ethyl]piperazine-1-carboxylate (100 mg, 0.2 mmol) in dichloromethane (2 mL) was added trifluoroacetic acid (89 mg, 0.8 mmol). The mixture was stirred at 25° C. for 0.5 h, then concentrated under reduced pressure to afford 2-[3-[3-[(4-methyl-1,2,4-triazol-3-yl)methyl]oxetan-3-yl]phenyl]-6-(1-piperazin-1-ylethyl)-4-(trifluoromethyl)isoindolin-1-one trifluoroacetate (90 mg, crude) as a colorless oil. MS (ESI) m/z: 541.3 [M+H]+

### Step 5: Preparation of 3-[4-fluoro-5-[4-[4-[[4-[1-[2-[3-[3-[(4-methyl-1,2,4-triazol-3-yl)methyl]oxetan-3-yl]phenyl]-3-oxo-7-(trifluoromethyl)isoindolin-5-yl]ethyl]piperazin-1-yl]methyl]cyclohexoxy]-1-piperidyl]-1-oxo-isoindolin-2-yl]piperidine-2,6-dione

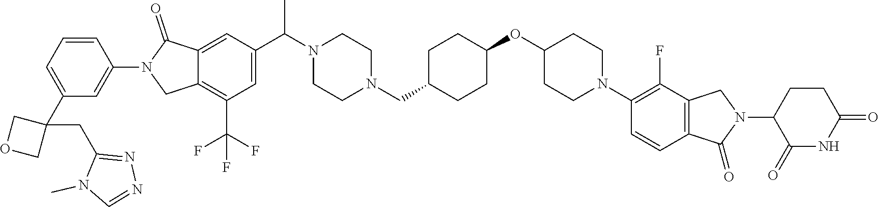 [SMILES: CC(c1cc2c(c(C(F)(F)F)c1)CN(c1cccc(C3(Cc4nncn4C)COC3)c1)C2=O)N1CCN(C[C@H]2CC[C@H](OC3CCN(c4ccc5c(c4F)CN(C4CCC(=O)NC4=O)C5=O)CC3)CC2)CC1]

To a solution of 2-[3-[3-[(4-methyl-1,2,4-triazol-3-yl)methyl]oxetan-3-yl]phenyl]-6-(1-piperazin-1-ylethyl)-4-(trifluoromethyl)isoindolin-1-one (85 mg, 0.2 mmol) in N,N-dimethylformamide (2 mL) was added 4-methylmorpholine (48 mg, 0.5 mmol) and 4-[[1-[2-(2,6-dioxo-3-piperidyl)-4-fluoro-1-oxo-isoindolin-5-yl]-4-piperidyl]oxy]cyclohexanecarbaldehyde (148 mg, 0.3 mmol). The reaction mixture was stirred at 0° C. for 0.5 h, then sodium triacetoxyborohydride (100 mg, 0.5 mmol) was added at 0° C. and the mixture was stirred at 25° C. for 10 h. The mixture was diluted with water (10 mL) and extracted with ethyl acetate (3×10 mL). The organic layers were concentrated under reduced pressure. The residue was purified by preparative HPLC (column: Phenomenex Luna C18 150*25 mm*10 um; mobile phase: [formic acid in water-acetonitrile]; B %: 18%-45%, 9 min) to afford 3-[4-fluoro-5-[4-[4-[[4-[1-[2-[3-[3-[(4-methyl-1,2,4-triazol-3-yl)methyl]oxetan-3-yl]phenyl]-3-oxo-7-(trifluoromethyl)isoindolin-5-yl]ethyl]piperazin-1-yl]methyl]cyclohexoxy]-1-piperidyl]-1-oxo-isoindolin-2-yl]piperidine-2,6-dione formate (92.3 mg, 56%) as white solid. MS (ESI) m/z: 996.3 [M+H]⁺; ¹H NMR (400 MHz, DMSO-d₆) δ 10.98 (s, 1H), 8.18 (s, 1H), 8.15 (s, 1H), 7.97 (s, 1H), 7.92-7.86 (m, 2H), 7.45 (d, J=8.0 Hz, 1H), 7.38-7.32 (m, 2H), 7.15 (t, J=8.0 Hz, 1H), 6.77 (d, J=8.0 Hz, 1H), 5.11-5.03 (m, 3H), 4.96 (d, J=6.0 Hz, 2H), 4.88 (d, J=6.0 Hz, 2H), 4.47 (d, J=16.8 Hz, 1H), 4.30 (d, J=16.8 Hz, 1H), 3.71 (d, J=6.8 Hz, 1H), 3.63-3.56 (m, 2H), 3.51 (s, 2H), 2.97-2.83 (m, 7H), 2.60 (d, J=2.4 Hz, 1H), 2.56 (s, 1H), 2.53 (d, J=6.8 Hz, 1H), 2.45-2.24 (m, 8H), 2.05 (d, J=7.2 Hz, 2H), 1.98-1.86 (m, 5H), 1.72 (d, J=11.2 Hz, 2H), 1.59-1.50 (m, 2H), 1.42-1.32 (m, 4H), 1.18-1.06 (m, 2H), 0.83 (q, J=12.0 Hz, 2H).

### Example 10: Exemplary synthesis of 3-[4-fluoro-1-oxo-5-(4-{[(1r, 4r)-4-[(4-{[(3S)-1-{[2-(3-{3-[(4-methyl-4H-1,2,4-triazol-3-yl)methyl]oxetan-3-yl}phenyl)-3-oxo-7-(trifluoromethyl)-2,3-dihydro-1H-isoindol-5-yl]methyl}pyrrolidin-3-yl]methyl}piperazin-1-yl)methyl]cyclohexyl]oxy}piperidin-1-yl)-2,3-dihydro-1H-isoindol-2-yl]piperidine-2,6-dione

### Step 1: Preparation of tert-butyl (3S)-3-(p-tolylsulfonyloxymethyl)pyrrolidine-1-carboxylate

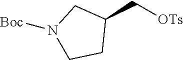 [SMILES: Cc1ccc(S(=O)(=O)OC[C@H]2CCN(C(=O)OC(C)(C)C)C2)cc1]

To a solution of tert-butyl (3S)-3-(hydroxymethyl)pyrrolidine-1-carboxylate (3.0 g, 15 mmol) in dichloromethane (50 mL) was added p-toluenesulfonyl chloride (8.5 g, 45 mmol) and triethylamine (4.5 g, 45 mmol) at 0° C. The mixture was stirred at 25° C. for 10 h, then concentrated under reduced pressure. The residue was purified by column chromatography (petroleum ether/ethyl acetate=30/1 to 3/1) to give tert-butyl (3S)-3-(p-tolylsulfonyloxymethyl)pyrrolidine-1-carboxylate (5.20 g, 98%) as a colorless oil. MS (ESI) m/z: 299.9 [M-56+H]⁺.

### Step 2: Preparation of benzyl 4-[[(3R)-1-tert-butoxycarbonylpyrrolidin-3-yl]methyl]piperazine-1-carboxylate

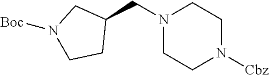 [SMILES: CC(C)(C)OC(=O)N1CC[C@H](CN2CCN(C(=O)OCc3ccccc3)CC2)C1]

To a solution of tert-butyl (3S)-3-(p-tolylsulfonyloxymethyl)pyrrolidine-1-carboxylate (5.2 g, 15 mmol) and benzyl piperazine-1-carboxylate (4.8 g, 22 mmol) in acetonitrile (60 mL) was added diisopropylethylamine (5.67 g, 44 mmol). The mixture was stirred at 80° C. for 10 h, then concentrated under reduced pressure. The residue was purified by column chromatography (petroleum ether/ethyl acetate=10/1 to 3/1) to give benzyl 4-[[(3R)-1-tert-butoxycarbonylpyrrolidin-3-yl]methyl]piperazine-1-carboxylate (4.30 g, 73%) as a brown oil. MS (ESI) m/z: 404.1 [M+H]⁺.

### Step 3: Preparation of tert-butyl (3R)-3-(piperazin-1-ylmethyl)pyrrolidine-1-carboxylate

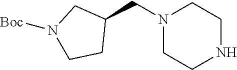 [SMILES: CC(C)(C)OC(=O)N1CC[C@H](CN2CCNCC2)C1]

To a solution of benzyl 4-[[(3R)-1-tert-butoxycarbonylpyrrolidin-3-yl]methyl]piperazine-1-carboxylate (4.2 g, 10 mmol) in methanol (60 mL) was added 5% palladium on activated carbon (500 mg) under nitrogen atmosphere. The suspension was degassed and purged with hydrogen three times, then stirred under hydrogen (15 Psi) at 25° C. for 5 h. The mixture was filtered and the filtrate was concentrated under reduced pressure to give tert-butyl (3R)-3-(piperazin-1-ylmethyl)pyrrolidine-1-carboxylate (2.7 g, 96%) as a colorless oil, which was used in the next step directly. ¹H NMR (400 MHz, DMSO-d₆) δ 3.41-3.24 (m, 3H), 3.21 (s, 1H), 3.14 (s, 1H), 2.92-2.83 (m, 1H), 2.66 (t, J=4.8 Hz, 3H), 2.44-2.30 (m, 2H), 2.26 (d, J=17.2 Hz, 2H), 2.19 (d, J=7.6 Hz, 2H), 1.88 (dd, J=6.4, 11.2 Hz, 1H), 1.56-1.45 (m, 1H), 1.39 (s, 9H).

### Step 4: Preparation of tert-butyl (3R)-3-[[4-[[4-[[1-[2-(2,6-dioxo-3-piperidyl)-4-fluoro-1-oxo-isoindolin-5-yl]-4-piperidyl]oxy]cyclohexyl]methyl]piperazin-1-yl]methyl]pyrrolidine-1-carboxylate

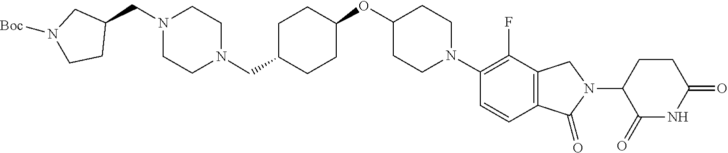 [SMILES: CC(C)(C)OC(=O)N1CC[C@H](CN2CCN(C[C@H]3CC[C@H](OC4CCN(c5ccc6c(c5F)CN(C5CCC(=O)NC5=O)C6=O)CC4)CC3)CC2)C1]

To a stirred solution of tert-butyl (3R)-3-(piperazin-1-ylmethyl)pyrrolidine-1-carboxylate (86 mg, 0.3 mmol) and 4-[[1-[2-(2,6-dioxo-3-piperidyl)-4-fluoro-1-oxo-isoindolin-5-yl]-4-piperidyl]oxy]cyclohexanecarbaldehyde (100 mg, 0.2 mmol) in dichloromethane (2 mL) was added sodium triacetoxyborohydride (90 mg, 0.4 mmol) at 0° C. The mixture was stirred at 25° C. for 10 h, then concentrated under reduced pressure. The residue was purified by preparative thin layer chromatography (dichloromethane:methanol=10:1) to give tert-butyl (3R)-3-[[4-[[4-[[1-[2-(2,6-dioxo-3-piperidyl)-4-fluoro-1-oxo-isoindolin-5-yl]-4-piperidyl]oxy]cyclohexyl]methyl]piperazin-1-yl]methyl]pyrrolidine-1-carboxylate (150 mg, 97%) as a white solid. MS (ESI) m/z: 725.6 [M+H]⁺.

### Step 5: Preparation of 3-[4-fluoro-1-oxo-5-[4-[4-[[4-[[(3S)-pyrrolidin-3-yl]methyl]piperazin-1-yl]methyl]cyclohexoxy]-1-piperidyl]isoindolin-2-yl]piperidine-2,6-dione

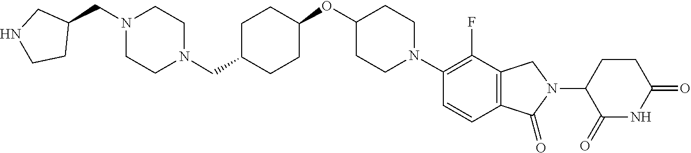 [SMILES: O=C1CCC(N2Cc3c(ccc(N4CCC(O[C@H]5CC[C@H](CN6CCN(C[C@H]7CCNC7)CC6)CC5)CC4)c3F)C2=O)C(=O)N1]

To a solution of tert-butyl (3R)-3-[[4-[[4-[[1-[2-(2,6-dioxo-3-piperidyl)-4-fluoro-1-oxo-isoindolin-5-yl]-4-piperidyl]oxy]cyclohexyl]methyl]piperazin-1-yl]methyl]pyrrolidine-1-carboxylate (75 mg, 0.1 mmol) in dichloromethane (1 mL) was added trifluoroacetic acid (36 mg, 0.3 mmol). The mixture was stirred at 25° C. for 0.5 h, then concentrated under reduced pressure to give 3-[4-fluoro-1-oxo-5-[4-[4-[[4-[[(3S)-pyrrolidin-3-yl]methyl]piperazin-1-yl]methyl]cyclohexoxy]-1-piperidyl]isoindolin-2-yl]piperidine-2,6-dione trifluoroacetate (75 mg, 98%) as a colorless oil. MS (ESI) m/z: 725.6 [M+H]⁺.

### Step 6: Preparation of 3-[4-fluoro-5-[4-[4-[[4-[[(3S)-1-[[2-[3-[3-[(4-methyl-1,2,4-triazol-3-yl)methyl]oxetan-3-yl]phenyl]-3-oxo-7-(trifluoromethyl)isoindolin-5-yl]methyl]pyrrolidin-3-yl]methyl]piperazin-1-yl]methyl]cyclohexoxy]-1-piperidyl]-1-oxo-isoindolin-2-yl]piperidine-2,6-dione

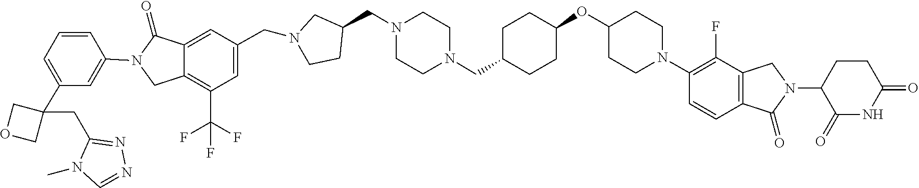 [SMILES: Cn1cnnc1CC1(c2cccc(N3Cc4c(cc(CN5CC[C@H](CN6CCN(C[C@H]7CC[C@H](OC8CCN(c9ccc%10c(c9F)CN(C9CCC(=O)NC9=O)C%10=O)CC8)CC7)CC6)C5)cc4C(F)(F)F)C3=O)c2)COC1]

To a solution of 3-[4-fluoro-1-oxo-5-[4-[4-[[4-[[(3S)-pyrrolidin-3-yl]methyl]piperazin-1-yl]methyl]cyclohexoxy]-1-piperidyl]isoindolin-2-yl]piperidine-2,6-dione trifluoroacetate (71 mg, 0.1 mmol) and 2-[3-[3-[(4-methyl-1,2,4-triazol-3-yl)methyl]oxetan-3-yl]phenyl]-3-oxo-7-(trifluoromethyl)isoindoline-5-carbaldehyde (40 mg, 0.1 mmol) in dichloromethane (1 mL) and N,N-dimethylformamide (1 mL) was added sodium cyanoborohydride (17 mg, 0.3 mmol) and triethylamine (27 mg, 0.3 mmol). Then tetraisopropoxytitanium (25 mg, 0.1 mmol) was added and the mixture was stirred at 25° C. for 10 h. The reaction mixture was concentrated, diluted with water (15 mL), and extracted with ethyl acetate (5 mL). The organic phase was washed with brine (10 mL), dried over anhydrous sodium sulfate, filtered, and concentrated. The residue was purified by preparative HPLC (column: Phenomenex Luna C18 150×25 mm×10 um; mobile phase: [formic acid in water-acetonitrile]; B %: 4%-34%, 10 min) to afford 3-[4-fluoro-5-[4-[4-[[4-[[(3S)-1-[[2-[3-[3-[(4-methyl-1,2,4-triazol-3-yl)methyl]oxetan-3-yl]phenyl]-3-oxo-7-(trifluoromethyl)isoindolin-5-yl]methyl]pyrrolidin-3-yl]methyl]piperazin-1-yl]methyl]cyclohexoxy]-1-piperidyl]-1-oxo-isoindolin-2-yl]piperidine-2,6-dione di-formate (7.1 mg, 6%) as a white solid. MS (ESI) m/z: 1065.8 [M+H]⁺; ¹H NMR (400 MHz, DMSO-d₆) δ 10.97 (s, 1H), 8.18 (s, 3H), 7.97 (s, 1H), 7.93 (s, 1H), 7.90-7.86 (m, 1H), 7.46 (d, J=8.4 Hz, 1H), 7.40 (s, 1H), 7.35 (t, J=8.0 Hz, 1H), 7.16 (t, J=8.0 Hz, 1H), 6.85-6.67 (m, 1H), 5.09 (s, 2H), 5.05 (d, J=5.6 Hz, 1H), 4.97 (d, J=6.0 Hz, 2H), 4.89 (d, J=6.0 Hz, 2H), 4.51-4.44 (m, 1H), 4.31 (d, J=16.8 Hz, 1H), 3.82-3.74 (m, 2H), 3.68-3.56 (m, 3H), 3.53-3.49 (m, 3H), 2.98-2.92 (m, 2H), 2.90 (s, 4H), 2.63-2.53 (m, 2H), 2.40 (s, 1H), 2.30-2.20 (m, 8H), 2.05-1.99 (m, 3H), 1.98-1.82 (m, 8H), 1.79-1.68 (m, 3H), 1.61-1.50 (m, 3H), 1.40 (dd, J=6.4, 13.2 Hz, 2H), 1.21-1.03 (m, 3H), 0.89-0.81 (m, 2H).

### Example 11: Exemplary synthesis of 2-(2,6-dioxo-3-piperidyl)-5-[2-[[1-[2-[3-[3-[(4-methyl-1,2,4-triazol-3-yl)methyl]oxetan-3-yl]phenyl]-3-oxo-7-(trifluoromethyl)isoindolin-5-yl]-4-piperidyl]methyl]-2,7-diazaspiro[3.5]nonan-7-yl]isoindoline-1,3-dione

### Step 1: Preparation of 5-(3,9-diazaspiro[5.5]undecan-3-yl)-2-(2,6-dioxo-3-piperidyl)isoindoline-1,3-dione

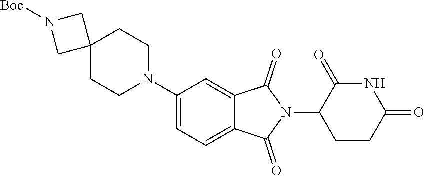 [SMILES: CC(C)(C)OC(=O)N1CC2(CCN(c3ccc4c(c3)C(=O)N(C3CCC(=O)NC3=O)C4=O)CC2)C1]

To a solution of 2-(2,6-dioxo-3-piperidyl)-5-fluoro-isoindoline-1,3-dione (100 mg, 0.4 mmol) in dimethylsulfoxide (1 mL) was added diisopropylethylamine (189 μL, 1.1 mmol) and tert-butyl 2,7-diazaspiro[3.5]nonane-2-carboxylate (98.32 mg, 0.4 mmol). The mixture was stirred at 100° C. for 12 h. The reaction mixture was filtered and concentrated under reduced pressure. The residue was purified by preparative HPLC (column: Phenomenex Luna C18 150×25 mm×10 um; mobile phase: [water (0.225% FA)-ACN]; B %: 41%-71%, 10 min) to afford tert-butyl 7-[2-(2,6-dioxo-3-piperidyl)-1,3-dioxo-isoindolin-5-yl]-2,7-diazaspiro[3.5]nonane-2-carboxylate (81.0 mg, 44%) as a yellow solid. MS (ESI) m/z: 483.2 [M+H]⁺; ¹HNMR (400 MHz, DMSO-d₆) δ 8.09 (brs, 1H), 7.69 (d, J=8.4 Hz, 1H), 7.29 (d, J=2.4 Hz, 1H), 7.07 (dd, J=8.4, 2.4 Hz, 1H), 4.94 (dd, J=12.4, 5.6 Hz, 1H), 3.71 (s, 4H), 3.35-3.45 (m, 4H), 2.69-2.98 (m, 3H), 2.08-2.21 (m, 1H), 1.86-1.92 (m, 4H), 1.46 (s, 9H).

### Step 2: Preparation of 5-(2,7-diazaspiro [3.5]nonan-7-yl)-2-(2,6-dioxo-3-piperidyl)isoindoline-1,3-dione

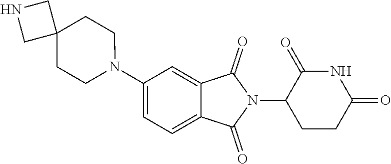 [SMILES: O=C1CCC(N2C(=O)c3ccc(N4CCC5(CC4)CNC5)cc3C2=O)C(=O)N1]

To a solution of tert-butyl 7-[2-(2,6-dioxo-3-piperidyl)-1,3-dioxo-isoindolin-5-yl]-2,7-diazaspiro[3.5]nonane-2-carboxylate (35 mg, 0.07 mmol) in dichloromethane (1.5 mL) was added trifluoroacetic acid (0.1 mL, 0.07 mmol). The mixture was stirred at 25° C. for 2 h, then concentrated to afford 5-(2,7-diazaspiro[3.5]nonan-7-yl)-2-(2,6-dioxo-3-piperidyl)isoindoline-1,3-dione trifluoroacetate (27 mg, crude) as a yellow oil. MS (ESI) m/z: 383.3 [M+H]⁺.

### Step 3: Preparation of 6-[4-(dimethoxymethyl)-1-piperidyl]-2-[3-[3-[(4-methyl-1,2,4-triazol-3-yl)methyl]oxetan-3-yl]phenyl]-4-(trifluoromethyl)isoindolin-1-one

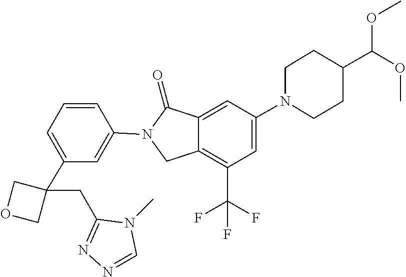 [SMILES: COC(OC)C1CCN(c2cc3c(c(C(F)(F)F)c2)CN(c2cccc(C4(Cc5nncn5C)COC4)c2)C3=O)CC1]

To a mixture of 6-bromo-2-[3-[3-[(4-methyl-1,2,4-triazol-3-yl)methyl]oxetan-3-yl]phenyl]-4-(trifluoromethyl)isoindolin-1-one (200 mg, 0.4 mmol), 4-(dimethoxymethyl) piperidine (75 mg, 0.5 mmol) and cesium carbonate (257 mg, 0.8 mmol) in dioxane (5 mL) was added XPhos Pd G4 (34 mg, 0.04 mmol). The mixture was stirred at 100° C. for 12 h, then filtered and concentrated. The residue was purified by preparative TLC (dichloromethane:methanol=10:1) to afford 6-[4-(dimethoxymethyl)-1-piperidyl]-2-[3-[3-[(4-methyl-1,2,4-triazol-3-yl) methyl]oxetan-3-yl]phenyl]-4-(trifluoromethyl)isoindolin-1-one (47 mg, 20%) as a yellow solid. MS (ESI) m/z: 586.4 [M+H]⁺.

### Step 4: Preparation of 5-(2,7-diazaspiro[3.5]nonan-7-yl)-2-(2,6-dioxo-3-piperidyl)isoindoline-1,3-dione

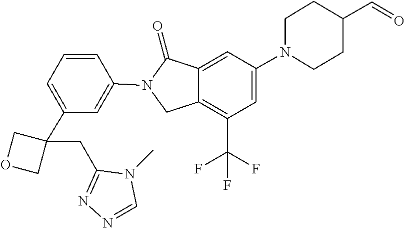 [SMILES: Cn1cnnc1CC1(c2cccc(N3Cc4c(cc(N5CCC(C=O)CC5)cc4C(F)(F)F)C3=O)c2)COC1]

To a solution of tert-butyl 7-[2-(2,6-dioxo-3-piperidyl)-1,3-dioxo-isoindolin-5-yl]-2,7-diazaspiro[3.5]nonane-2-carboxylate (35 mg, 0.07 mmol) in dichloromethane (1.5 mL) was added trifluoroacetic acid (0.1 mL, 0.07 mmol). The mixture was stirred at 25° C. for 2 h, then concentrated to afford 5-(2,7-diazaspiro[3.5]nonan-7-yl)-2-(2,6-dioxo-3-piperidyl) isoindoline-1,3-dione (27 mg, 97%) as a yellow oil.

### Step 5: Preparation of 2-(2,6-dioxo-3-piperidyl)-5-[2-[[1-[2-[3-[3-[(4-methyl-1,2,4-triazol-3-yl)methyl]oxetan-3-yl]phenyl]-3-oxo-7-(trifluoromethyl)isoindolin-5-yl]-4-piperidyl]methyl]-2,7-diazaspiro[3.5]nonan-7-yl]isoindoline-1,3-dione

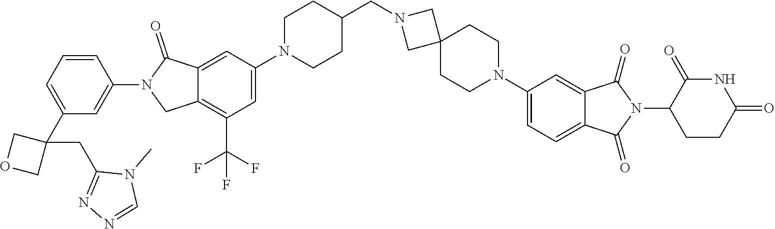 [SMILES: Cn1cnnc1CC1(c2cccc(N3Cc4c(cc(N5CCC(CN6CC7(CCN(c8ccc9c(c8)C(=O)N(C8CCC(=O)NC8=O)C9=O)CC7)C6)CC5)cc4C(F)(F)F)C3=O)c2)COC1]

To a solution of 1-[2-[3-[3-[(4-methyl-1,2,4-triazol-3-yl)methyl]oxetan-3-yl]phenyl]-3-oxo-7-(trifluoromethyl)isoindolin-5-yl]piperidine-4-carbaldehyde (43 mg, 0.08 mmol) and 5-(2,7-diazaspiro[3.5]nonan-7-yl)-2-(2,6-dioxo-3-piperidyl)isoindoline-1,3-dione (25 mg, 0.07 mmol) in dichloromethane (2 mL) was added trimethylamine (0.1 mL, 0.1 mmol). The mixture was stirred at 25° C. for 1 h, then sodium triacetoxyborohydride (28 mg, 0.1 mmol) was added to the reaction and stirred for 2 h. The reaction mixture was diluted with water (50 mL) and extracted with dichloromethane (20 mL×3). The combined organic phase was washed with saturated brine (50 mL), dried over anhydrous sodium sulfate, filtered, and concentrated under vacuum. The crude product was purified by reversed-phase HPLC (column: Unisil 3-100 C18 Ultra 150×50 mm×3 um; mobile phase: [water(FA)-ACN]; B %: 12%-42%, 7 min) to afford 2-(2,6-dioxo-3-piperidyl)-5-[2-[[1-[2-[3-[3-[(4-methyl-1,2,4-triazol-3-yl)methyl]oxetan-3-yl]phenyl]-3-oxo-7-(trifluoromethyl)isoindolin-5-yl]-4-piperidyl]methyl]-2,7-diazaspiro[3.5]nonan-7-yl]isoindoline-1,3-dione (23 mg, 37%) as a yellow solid. MS (ESI) m/z: 453.6 [1/2M+H]⁺; ¹H NMR (400 MHz, DMSO-d₆) δ 11.07 (s, 1H), 8.19 (d, J=2.8 Hz, 2H), 7.90-7.86 (m, 1H), 7.65 (d, J=8.4 Hz, 1H), 7.47 (s, 1H), 7.42 (s, 1H), 7.36-7.30 (m, 3H), 7.24 (dd, J=2.0, 8.8 Hz, 1H), 6.75 (d, J=7.6 Hz, 1H), 5.06 (dd, J=5.2, 12.8 Hz, 1H), 4.98-4.93 (m, 4H), 4.88 (d, J=6.0 Hz, 2H), 3.86 (d, J=12.0 Hz, 2H), 3.50 (s, 3H), 3.43 (d, J=3.2 Hz, 2H), 3.04 (s, 4H), 2.89 (s, 3H), 2.86-2.75 (m, 3H), 2.62-2.55 (m, 1H), 2.52 (d, J=2.0 Hz, 2H), 2.38 (d, J=6.8 Hz, 2H), 2.01 (td, J=5.2, 10.4 Hz, 1H), 1.82-1.72 (m, 6H), 1.56-1.44 (m, 1H), 1.28-1.19 (m, 2H).

### Example 12: Exemplary synthesis of 3-{4-fluoro-7-methyl-5-[4-({2-[2-(3-{3-[(4-methyl-4H-1,2,4-triazol-3-yl)methyl]oxetan-3-yl}phenyl)-3-oxo-7-(trifluoromethyl)-2,3-dihydro-1H-isoindol-5-yl]-2,7-diazaspiro [3.5]nonan-7-yl}methyl)piperidin-1-yl]-1-oxo-2,3-dihydro-1H-isoindol-2-yl}piperidine-2,6-dione

### Step 1: Preparation of 5-bromo-4-fluoro-3-hydroxy-7-methyl-3H-isobenzofuran-1-one

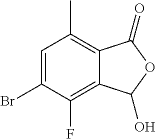

To a solution of 4-bromo-5-fluoro-2-methyl-benzoic acid (1.90 g, 8 mmol) in tetrahydrofuran (20 mL) was added lithium diisopropylamide (2 M, 12 mL) dropwise at −50° C. under nitrogen atmosphere. The mixture was stirred for 2 h at −50° C., then N,N-dimethylformamide (2.98 g, 41 mmol) was added dropwise at −50° C. The reaction was warmed up to −20° C. and stirred for 1 h, quenched with saturated ammonium chloride solution (30 mL) and extracted with ethyl acetate (30 mL×2). The combined organic layers were washed with brine (30 mL), dried over anhydrous sodium sulfate, filtered, and concentrated. The residue was purified by column chromatography (petroleum ether/ethyl acetate=5/1 to 3/1) to afford 5-bromo-4-fluoro-3-hydroxy-7-methyl-3H-isobenzofuran-1-one (0.90 g, 42%) as a white solid.

### Step 2: Preparation of 3-(5-bromo-4-fluoro-7-methyl-1-oxo-isoindolin-2-yl)piperidine-2,6-dione

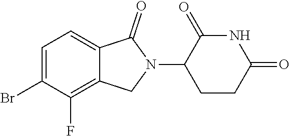 [SMILES: O=C1CCC(N2Cc3c(ccc(Br)c3F)C2=O)C(=O)N1]

To a solution of 5-bromo-4-fluoro-3-hydroxy-7-methyl-3H-isobenzofuran-1-one (800 mg, 3.1 mmol) and 3-aminopiperidine-2,6-dione hydrochloride (757 mg, 4.6 mmol) in N,N-dimethylformamide (8 mL) was added sodium triacetoxyborohydride (1.62 g, 7.7 mmol). The mixture was stirred at 25° C. for 12 h and diluted with dichloromethane (30 mL) and ammonium chloride aqueous (50 mL). The organic layer was separated, and the aqueous layer was extracted with ethyl acetate (2×30 mL). The combined organic extracts were washed with brine (50 mL), dried over anhydrous sodium sulfate, filtered, and concentrated. The residue was purified by column chromatography (petroleum ether/ethyl acetate=10/1 to 1/1) to afford 3-(5-bromo-4-fluoro-7-methyl-1-oxo-isoindolin-2-yl)piperidine-2,6-dione (230 mg, 21%) as a white solid. MS (ESI) m/z: 355.0 [M+H]⁺.

### Step 3: Preparation of 3-[5-[4-(dimethoxymethyl)-1-piperidyl]-4-fluoro-7-methyl-1-oxo-isoindolin-2-yl]piperidine-2,6-dione

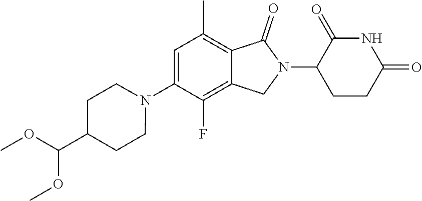 [SMILES: COC(OC)C1CCN(c2cc(C)c3c(c2F)CN(C2CCC(=O)NC2=O)C3=O)CC1]

A mixture of 3-(5-bromo-4-fluoro-7-methyl-1-oxo-isoindolin-2-yl)piperidine-2,6-dione (180 mg, 0.5 mmol), 4-(dimethoxymethyl)piperidine (121 mg, 0.8 mmol), cesium carbonate (495 mg, 1.5 mmol) and 1,3-bis[2,6-bis(1-propylbutyl)phenyl]-4,5-dichloro-2H-imidazol-1-ium-2-ide; 3-chloropyridine; dichloropalladium (49 mg, 0.05 mmol) in N,N-dimethylformamide (3 mL) was degassed and purged with nitrogen three times, then the mixture was stirred at 100° C. for 2 h under nitrogen. The reaction mixture was diluted with brine (20 mL) and extracted with dichloromethane (15 mL×2). The combined organic layers were dried over anhydrous sodium sulfate, filtered, and concentrated. The residue was purified by column chromatography (dichloromethane/methanol=50/1 to 15/1) to afford 3-[5-[4-(dimethoxymethyl)-1-piperidyl]-4-fluoro-7-methyl-1-oxo-isoindolin-2-yl]piperidine-2,6-dione (70 mg, 32%) as a white solid. MS (ESI) m/z: 434.1 [M+H]⁺.

### Step 4: Preparation of 1-[2-(2,6-dioxo-3-piperidyl)-4-fluoro-7-methyl-1-oxo-isoindolin-5-yl]piperidine-4-carbaldehyde

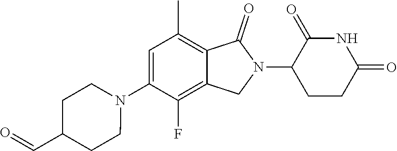 [SMILES: Cc1cc(N2CCC(C=O)CC2)c(F)c2c1C(=O)N(C1CCC(=O)NC1=O)C2]

To a solution of 3-[5-[4-(dimethoxymethyl)-1-piperidyl]-4-fluoro-7-methyl-1-oxo-isoindolin-2-yl]piperidine-2,6-dione (60 mg, 0.1 mmol) in dichloromethane (1 mL) was added trifluoroacetic acid (770 mg, 6.8 mmol). The mixture was stirred at 25° C. for 0.5 h, then concentrated under reduced pressure to afford 1-[2-(2,6-dioxo-3-piperidyl)-4-fluoro-7-methyl-1-oxo-isoindolin-5-yl]piperidine-4-carbaldehyde (60 mg, crude) as a colorless oil. MS (ESI) m/z: 388.1 [M+H]⁺.

### Step 5: Preparation of 3-[4-fluoro-7-methyl-5-[4-[[2-[2-[3-[3-[(4-methyl-1,2,4-triazol-3-yl)methyl]oxetan-3-yl]phenyl]-3-oxo-7-(trifluoromethyl)isoindolin-5-yl]-2,7-diazaspiro[3.5]nonan-7-yl]methyl]-1-piperidyl]-1-oxo-isoindolin-2-yl]piperidine-2,6-dione

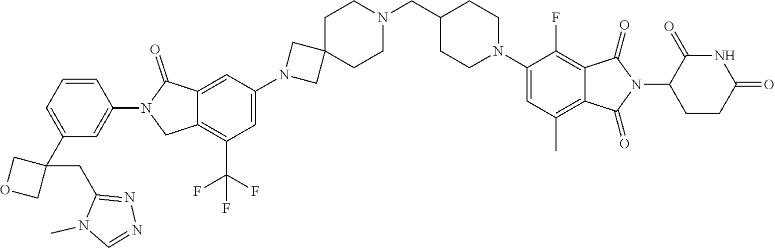 [SMILES: Cc1cc(N2CCC(CN3CCC4(CC3)CN(c3cc5c(c(C(F)(F)F)c3)CN(c3cccc(C6(Cc7nncn7C)COC6)c3)C5=O)C4)CC2)c(F)c2c1C(=O)N(C1CCC(=O)NC1=O)C2=O]

To a solution of 6-(2,7-diazaspiro[3.5]nonan-2-yl)-2-[3-[3-[(4-methyl-1,2,4-triazol-3-yl)methyl]oxetan-3-yl]phenyl]-4-(trifluoromethyl)isoindolin-1-one trifluoroacetate (80 mg, 0.1 mmol) and 1-[2-(2,6-dioxo-3-piperidyl)-4-fluoro-7-methyl-1-oxo-isoindolin-5-yl]piperidine-4-carbaldehyde trifluoroacetate (60 mg, 0.1 mmol) in dichloromethane (3 mL) was added triethylamine (24 mg, 0.2 μmol). The mixture was stirred at 25° C. for 1 h, upon which sodium triacetoxyborohydride (76 mg, 0.4 mmol) was added and stirred at 25° C. for 1 h. The reaction mixture was filtered and concentrated. The residue was purified by preparative HPLC (column: Phenomenex Luna C18 150×25 mm×10 um; mobile phase: [water(FA)-ACN]; B %: 12%-42%, 10 min) to afford 3-[4-fluoro-7-methyl-5-[4-[[2-[2-[3-[3-[(4-methyl-1,2,4-triazol-3-yl)methyl]oxetan-3-yl]phenyl]-3-oxo-7-(trifluoromethyl)isoindolin-5-yl]-2,7-diazaspiro[3.5]nonan-7-yl]methyl]-1-piperidyl]-1-oxo-isoindolin-2-yl]piperidine-2,6-dione (49 mg, 43%) as a white solid. MS (ESI) m/z: 924.3 [M+H]⁺; ¹H NMR (400 MHz, DMSO-d₆) δ 10.97 (s, 1H), 8.19 (s, 1H), 7.89 (dd, J=1.6, 8.0 Hz, 1H), 7.38-7.31 (m, 2H), 6.94 (s, 2H), 6.90 (d, J=7.6 Hz, 1H), 6.75 (d, J=7.2 Hz, 1H), 5.03 (dd, J=5.2, 13.2 Hz, 1H), 4.99-4.93 (m, 4H), 4.89 (d, J=6.0 Hz, 2H), 4.40 (d, J=16.8 Hz, 1H), 4.24 (d, J=16.8 Hz, 1H), 3.70 (s, 4H), 3.51 (s, 4H), 3.49 (d, J=1.6 Hz, 2H), 2.91 (s, 3H), 2.77 (t, J=11.2 Hz, 2H), 2.61 (d, J=3.2 Hz, 2H), 2.54 (s, 3H), 2.41 (dd, J=4.4, 12.8 Hz, 3H), 2.18 (d, J=7.2 Hz, 2H), 1.99-1.94 (m, 1H), 1.83 (s, 1H), 1.79 (m, 5H), 1.73-1.67 (m, 1H), 1.32-1.22 (m, 2H).

### Example 13: Exemplary synthesis of (1s, 3s)-3-[(4-methyl-4H-1,2,4-triazol-3-yl)methyl]-3-[3-(1-oxo-6-{[(3S)-3-[(4-{[(1r, 4r)-4-({1-[2-(2,6-dioxopiperidin-3-yl)-4-fluoro-1-oxo-2,3-dihydro-1H-isoindol-5-yl]piperidin-4-yl}oxy)cyclohexyl]methyl}piperazin-1-yl)methyl]piperidin-1-yl]methyl}-4-(trifluoromethyl)-2,3-dihydro-1H-isoindol-2-yl)phenyl]cyclobutane-1-carbonitrile

### Step 1: Preparation of 3-[3-[6-formyl-1-oxo-4-(trifluoromethyl)isoindolin-2-yl]phenyl]-3-[(4-methyl-1,2,4-triazol-3-yl)methyl]cyclobutanecarbonitrile

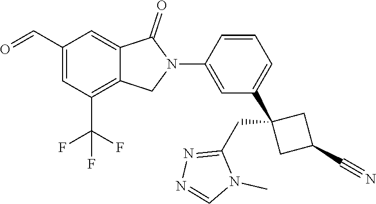 [SMILES: Cn1cnnc1C[C@]1(c2cccc(N3Cc4c(cc(C=O)cc4C(F)(F)F)C3=O)c2)C[C@@H](C#N)C1]

To a solution of 3-(3-aminophenyl)-3-[(4-methyl-1,2,4-triazol-3-yl)methyl]cyclobutanecarbonitrile (530 mg, 2 mmol) and methyl 2-(bromomethyl)-5-formyl-3-(trifluoromethyl)benzoate (670 mg, 2.1 mmol) in acetonitrile (20 mL) and water (10 mL) was added silver nitrate (438 mg, 2.6 mmol) in water (2.5 mL) at 0° C. The reaction was stirred for 12 h at 25° C., then solid sodium bicarbonate was added until the solution pH was 8. The mixture was then filtered through Celite and washed with acetonitrile (10 mL) followed by dichloromethane/ethyl acetate (9:1, 50 mL). The organic layer was separated, dried over sodium sulfate, and concentrated. The residue was purified by preparative (dichloromethane:methanol=15:1) to give 3-[3-[6-formyl-1-oxo-4-(trifluoromethyl)isoindolin-2-yl]phenyl]-3-[(4-methyl-1,2,4-triazol-3-yl)methyl]cyclobutanecarbonitrile (290 mg, 31%) as a yellow solid. MS (ESI) m/z: 480.2 [M+H]⁺.

### Step 2: Preparation of 3-[3-[6-[[(3S)-3-[[4-[[4-[[1-[2-(2,6-dioxo-3-piperidyl)-4-fluoro-1-oxo-isoindolin-5-yl]-4-piperidyl]oxy]cyclohexyl]methyl]piperazin-1-yl]methyl]-1-piperidyl]methyl]-1-oxo-4-(trifluoromethyl)isoindolin-2-yl]phenyl]-3-[(4-methyl-1,2,4-triazol-3-yl)methyl]cyclobutanecarbonitrile

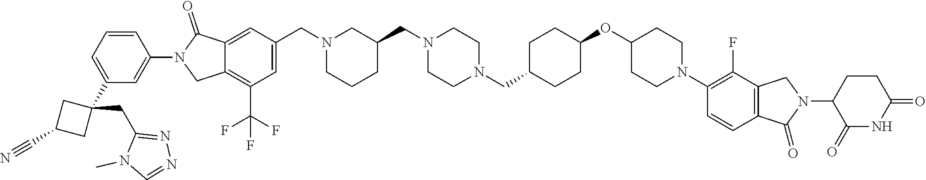 [SMILES: Cn1cnnc1C[C@]1(c2cccc(N3Cc4c(cc(CN5CCC[C@H](CN6CCN(C[C@H]7CC[C@H](OC8CCN(c9ccc%10c(c9F)CN(C9CCC(=O)NC9=O)C%10=O)CC8)CC7)CC6)C5)cc4C(F)(F)F)C3=O)c2)C[C@@H](C#N)C1]

To a solution of 3-[3-[6-formyl-1-oxo-4-(trifluoromethyl)isoindolin-2-yl]phenyl]-3-[(4-methyl-1,2,4-triazol-3-yl)methyl]cyclobutanecarbonitrile (76 mg, 0.2 mmol), 3-[4-fluoro-1-oxo-5-[4-[4-[[4-[[(3S)-3-piperidyl]methyl]piperazin-1-yl]methyl]cyclohexoxy]-1-piperidyl]isoindolin-2-yl]piperidine-2,6-dione trifluoroacetate (120 mg, 0.2 mmol), triethylamine (16 mg, 0.2 mmol) and tetraisopropoxytitanium (45 mg, 0.2 mmol) in dichloromethane (1 mL) and N,N-dimethylformamide (1 mL) was added sodium cyanoborohydride (20 mg, 0.3 mmol). The mixture was stirred at 25° C. for 4 h, then filtered and concentrated. The residue was purified by preparative HPLC (column: Phenomenex Luna C18 150×25 mm×10 um; mobile phase: [water(FA)-ACN]; B %: 14%-44%, 10 min) to give 3-[3-[6-[[(3S)-3-[[4-[[4-[[1-[2-(2,6-dioxo-3-piperidyl)-4-fluoro-1-oxo-isoindolin-5-yl]-4-piperidyl]oxy]cyclohexyl]methyl]piperazin-1-yl]methyl]-1-piperidyl]methyl]-1-oxo-4-(trifluoromethyl)isoindolin-2-yl]phenyl]-3-[(4-methyl-1,2,4-triazol-3-yl)methyl]cyclobutanecarbonitrile formate (23.1 mg, 12%) as a colorless solid. MS (ESI) m/z: 1103.8 [M+H]⁺; ¹H NMR, (400 MHz, DMSO-d₆) δ 10.99 (s, 1H), 8.15 (d, J=4.8 Hz, 2H), 7.92-8.06 (m, 2H), 7.88 (br d, J=7.6 Hz, 1H), 7.47 (d, J=8.0 Hz, 1H), 7.34 (t, J=8.0 Hz, 1H), 7.26 (s, 1H), 7.17 (br t, J=8.0 Hz, 1H), 6.68 (br d, J=6.8 Hz, 1H), 6.46-6.61 (m, 1H), 5.04-5.14 (m, 3H), 4.44-4.52 (m, 1H), 4.31 (br d, J=17.2 Hz, 1H), 3.69-3.86 (m, 3H), 3.56-3.67 (m, 2H), 3.01-3.11 (m, 3H), 2.82-2.99 (m, 6H), 2.71 (s, 5H), 2.57-2.66 (m, 6H), 2.38-2.45 (m, 4H), 1.86-2.02 (m, 9H), 1.66-1.80 (m, 5H), 1.42-1.62 (m, 6H), 1.08-1.23 (m, 3H), 0.84-1.06 ppm (m, 4H).

### Example 14: Exemplary synthesis of (1s, 3s)-3-[(4-methyl-4H-1,2,4-triazol-3-yl)methyl]-3-(3-{1-oxo-6-[(4-{[(1r, 4r)-4-({1-[2-(2,6-dioxopiperidin-3-yl)-4-fluoro-1-oxo-2,3-dihydro-1H-isoindol-5-yl]piperidin-4-yl}oxy)cyclohexyl]methyl}piperazin-1-yl)methyl]-4-(trifluoromethyl)-2,3-dihydro-1H-isoindol-2-yl}phenyl)cyclobutane-1-carbonitrile

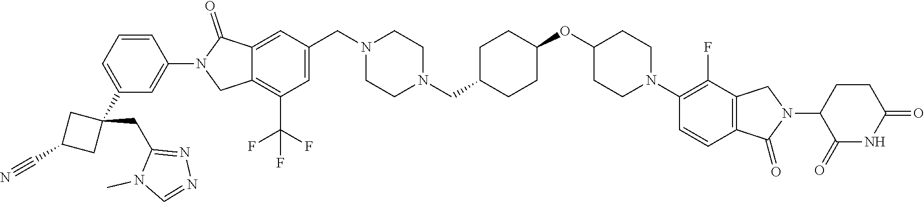 [SMILES: Cn1cnnc1C[C@]1(c2cccc(N3Cc4c(cc(CN5CCN(C[C@H]6CC[C@H](OC7CCN(c8ccc9c(c8F)CN(C8CCC(=O)NC8=O)C9=O)CC7)CC6)CC5)cc4C(F)(F)F)C3=O)c2)C[C@@H](C#N)C1]

To a solution of 3-[3-[6-formyl-1-oxo-4-(trifluoromethyl)isoindolin-2-yl]phenyl]-3-[(4-methyl-1,2,4-triazol-3-yl)methyl]cyclobutanecarbonitrile (88 mg, 0.2 mmol), 3-[4-fluoro-1-oxo-5-[4-[4-(piperazin-1-ylmethyl)cyclohexoxy]-1-piperidyl]isoindolin-2-yl]piperidine-2,6-dione trifluoroacetate (120 mg, 0.2 mmol), triethylamine (19 mg, 0.2 mmol) and tetraisopropoxytitanium (52 mg, 0.2 mmol) in dichloromethane (1 mL) and N,N-dimethylformamide (1 mL) was added sodium cyanoborohydride (23 mg, 0.4 mmol). The mixture was stirred at 25° C. for 4 h, then filtered and concentrated. The residue was purified by preparative HPLC (column: Phenomenex Luna C18 150×25 mm×10 um; mobile phase: [water(FA)-ACN]; B %: 21%-51%, 10 min) to give 3-[3-[6-[[4-[[4-[[1-[2-(2,6-dioxo-3-piperidyl)-4-fluoro-1-oxo-isoindolin-5-yl]-4-piperidyl]oxy]cyclohexyl]methyl]piperazin-1-yl]methyl]-1-oxo-4-(trifluoromethyl)isoindolin-2-yl]phenyl]-3-[(4-methyl-1,2,4-triazol-3-yl)methyl]cyclobutanecarbonitrile formate (23.0 mg, 12%) as a colorless solid. MS (ESI) m/z: 1006.8 [M+H]⁺; ¹H NMR (400 MHz, DMSO-d₆) δ 10.84-11.18 (m, 1H), 8.15 (s, 1H), 7.93-8.01 (m, 2H), 7.88 (dd, J=8.0, 1.6 Hz, 1H), 7.47 (d, J=8.0 Hz, 1H), 7.33 (t, J=8.0 Hz, 1H), 7.26 (s, 1H), 7.17 (t, J=8.4 Hz, 1H), 6.67 (br d, J=7.6 Hz, 1H), 5.02-5.12 (m, 3H), 4.48 (d, J=16.8 Hz, 1H), 4.31 (d, J=17.2 Hz, 1H), 3.70-3.86 (m, 3H), 3.56-3.67 (m, 1H), 3.24 (s, 2H), 3.06 (br t, J=10.4 Hz, 2H), 2.87-2.98 (m, 3H), 2.70 (s, 3H), 2.57-2.66 (m, 7H), 2.37-2.42 (m, 4H), 1.88-2.03 (m, 8H), 1.72-1.81 (m, 3H), 1.49-1.63 (m, 4H), 1.09-1.22 (m, 3H), 0.81-0.99 ppm (m, 3H).

### Example 15: Exemplary synthesis of 2-(2,6-dioxo-3-piperidyl)-5-[2-[[1-[2-[3-[3-[(4-methyl-1,2,4-triazol-3-yl)methyl]oxetan-3-yl]phenyl]-3-oxo-7-(trifluoromethyl)isoindolin-5-yl]-4-piperidyl]methoxy]ethoxy]isoindoline-1,3-dione

### Step 1: Preparation of 2-benzyloxyethyl 4-methylbenzenesulfonate

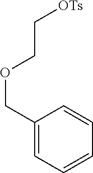 [SMILES: Cc1ccc(S(=O)(=O)OCCOCc2ccccc2)cc1]

To a solution of 2-benzyloxyethanol (17.00 g, 112 mmol) in dichloromethane (200 mL) was added triethylamine (33.91 g, 335 mmol), dimethylaminopyridine (2.73 g, 22 mmol) and p-toluenesulfonyl chloride (31.94 g, 168 mmol). The mixture was stirred at 25° C. for 2 h, then concentrated. The residue was purified by column chromatography (petroleum ether/ethyl acetate=30/1 to 8/1) to afford 2-benzyloxyethyl 4-methylbenzenesulfonate (21.50 g, 63%) as a colorless oil. ¹H NMR (400 MHz, DMSO-d₆) δ 7.82 (d, J=8.4 Hz, 2H), 7.38-7.27 (m, 7H), 4.51 (s, 2H), 4.25-4.20 (m, 2H), 3.71-3.67 (m, 2H), 2.46 (s, 3H).

### Step 2: Preparation of tert-butyl 4-(2-benzyloxyethoxymethyl)piperidine-1-carboxylate

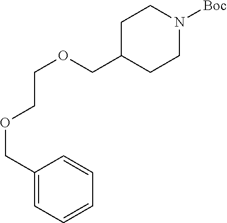 [SMILES: CC(C)(C)OC(=O)N1CCC(COCCOCc2ccccc2)CC1]

To a solution of tert-butyl 4-(hydroxymethyl)piperidine-1-carboxylate (3.51 g, 16 mmol) in tetrahydrofuran (50 mL) was added 60% sodium hydride (979 mg, 24 mmol) at 0° C. The reaction mixture was stirred at 0° C. for 0.5 h and then 2-benzyloxyethyl 4-methylbenzenesulfonate (5.00 g, 16 mmol) was added. The reaction mixture was stirred at 25° C. for 2 h, then quenched with saturated ammonium chloride solution (20 mL) at 0° C. The resulting mixture was extracted with ethyl acetate (100 mL). The organic layer was dried over anhydrous sodium sulfate, filtered, and concentrated. The residue was purified by silica gel column chromatography (petroleum ether/ethyl acetate=20/1 to 5/1) to afford tert-butyl 4-(2-benzyloxyethoxymethyl)piperidine-1-carboxylate (3.30 g, 58%) as a colorless oil. MS (ESI) m/z: 372.0 [M+Na]⁺; ¹H NMR (400 MHz, CDCl₃) δ 7.36 (d, J=4.4 Hz, 4H), 7.32-7.26 (m, 1H), 4.59 (s, 2H), 4.12 (d, J=7.2 Hz, 2H), 3.65-3.61 (m, 4H), 3.33 (d, J=6.4 Hz, 2H), 2.71 (t, J=12.0 Hz, 2H), 1.80-1.70 (m, 3H), 1.47 (s, 9H), 1.20-1.10 (m, 2H).

### Step 3: Preparation of tert-butyl 4-(2-hydroxyethoxymethyl)piperidine-1-carboxylate

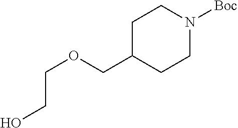 [SMILES: CC(C)(C)OC(=O)N1CCC(COCCO)CC1]

To a solution of tert-butyl 4-(2-benzyloxyethoxymethyl)piperidine-1-carboxylate (3.30 g, 9 mmol) in methanol (30 mL) was added 10% palladium on activated carbon (0.50 g) under nitrogen atmosphere. The suspension was degassed under vacuum and purged with hydrogen atmosphere several times. The mixture was stirred under hydrogen (50 psi) at 50° C. for 5 h, then filtered through Celite and concentrated. The residue was purified by column chromatography (petroleum ether/ethyl acetate=20/1 to 3/1) to afford tert-butyl 4-(2-hydroxyethoxymethyl)piperidine-1-carboxylate (1.69 g, 69%) as a colorless oil. ¹H NMR (400 MHz, CDCl₃) δ 4.15-4.04 (m, 2H), 3.74-3.69 (m, 2H), 3.54-3.50 (m, 2H), 3.31 (d, J=6.4 Hz, 2H), 2.68 (t, J=12.0 Hz, 2H), 2.09-2.03 (m, 1H), 1.75-1.65 (m, 2H), 1.44 (s, 9H), 1.17-1.10 (m, 2H).

### Step 4: Preparation of tert-butyl 4-(2-acetoxyethoxymethyl)piperidine-1-carboxylate

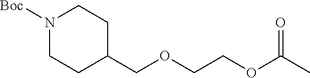 [SMILES: CC(=O)OCCOCC1CCN(C(=O)OC(C)(C)C)CC1]

To a solution of tert-butyl 4-(2-hydroxyethoxymethyl)piperidine-1-carboxylate (2.00 g, 7.7 mmol) and triethylamine (2.34 g, 23.1 mmol) in dichloromethane (20 mL) was added acetyl chloride (1.21 mg, 15.4 mmol). The mixture was stirred at 25° C. for 10 h, then quenched with water (30 mL) and extracted with dichloromethane (30 mL×2). The combined organic phase was washed with brine (30 mL), dried over anhydrous sodium sulfate, filtered, and concentrated. The residue was purified by column chromatography (petroleum ether/ethyl acetate=30/1 to 10/1) to afford tert-butyl 4-(2-acetoxyethoxymethyl)piperidine-1-carboxylate (2.20 g, 95%) as a yellow oil. ¹H NMR (400 MHz, CDCl₃) δ 4.22-4.16 (m, 2H), 4.08 (s, 2H), 3.63-3.57 (m, 2H), 3.30 (d, J=6.0 Hz, 2H), 2.73-2.62 (m, 2H), 2.07 (s, 3H), 1.76-1.64 (m, 3H), 1.44 (s, 9H), 1.18-1.06 (m, 2H).

### Step 5: Preparation of 2-(4-piperidylmethoxy)ethyl acetate

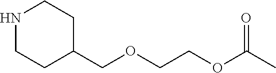

To a solution of tert-butyl 4-(2-acetoxyethoxymethyl)piperidine-1-carboxylate (500 mg, 1.7 mmol) in dichloromethane (5 mL) was added trifluoroacetic acid (2.31 g, 20.3 mmol). The mixture was stirred at 25° C. for 0.5 h, then concentrated under reduced pressure to afford 2-(4-piperidylmethoxy)ethyl acetate trifluoroacetate (500 mg, 96%) as a colorless oil. MS (ESI) m/z: 202.1 [M+H]⁺.

### Step 6: Preparation of 2-[[1-[2-[3-[3-[(4-methyl-1,2,4-triazol-3-yl)methyl]oxetan-3-yl]phenyl]-3-oxo-7-(trifluoromethyl)isoindolin-5-yl]-4-piperidyl]methoxy]ethyl acetate

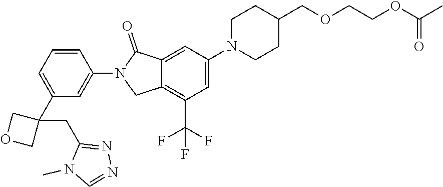 [SMILES: CC(=O)OCCOCC1CCN(c2cc3c(c(C(F)(F)F)c2)CN(c2cccc(C4(Cc5nncn5C)COC4)c2)C3=O)CC1]

A mixture of 6-bromo-2-[3-[3-[(4-methyl-1,2,4-triazol-3-yl)methyl]oxetan-3-yl]phenyl]-4-(trifluoromethyl)isoindolin-1-one (100 mg, 0.2 mmol), 2-(4-piperidylmethoxy)ethyl acetate trifluoroacetate (124 mg, 0.4 mmol), cesium carbonate (193 mg, 0.6 mmol) and XPhos Pd G4 (51 mg, 0.06 mmol) in dioxane (4 mL) was degassed and purged with nitrogen three times, then the mixture was stirred at 90° C. for 12 h under nitrogen atmosphere. The reaction mixture was diluted with brine (20 ml) and extracted with dichloromethane (20 mL×2). The combined organic layers were dried over anhydrous sodium sulfate, filtered, and concentrated under reduced pressure. The residue was purified by preparative TLC (dichloromethane/methanol=10/1) to afford 2-[[1-[2-[3-[3-[(4-methyl-1,2,4-triazol-3-yl)methyl]oxetan-3-yl]phenyl]-3-oxo-7-(trifluoromethyl)isoindolin-5-yl]-4-piperidyl]methoxy]ethyl acetate (70 mg, 57%) as a light yellow solid. MS (ESI) m/z: 628.2 [M+H]⁺.

### Step 7: Preparation of 6-[4-(2-hydroxyethoxymethyl)-1-piperidyl]-2-[3-[3-[(4-methyl-1,2,4-triazol-3-yl)methyl]oxetan-3-yl]phenyl]-4-(trifluoromethyl)isoindolin-1-one

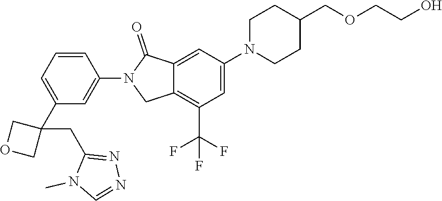 [SMILES: Cn1cnnc1CC1(c2cccc(N3Cc4c(cc(N5CCC(COCCO)CC5)cc4C(F)(F)F)C3=O)c2)COC1]

To a solution of 2-[[1-[2-[3-[3-[(4-methyl-1,2,4-triazol-3-yl)methyl]oxetan-3-yl]phenyl]-3-oxo-7-(trifluoromethyl)isoindolin-5-yl]-4-piperidyl]methoxy]ethyl acetate (70 mg, 0.1 mmol) in methanol (3 mL) and water (1 mL) was added sodium hydroxide (13 mg, 0.3 mmol). The mixture was stirred at 25° C. for 1 h, then acidified to pH 5-6 with hydrochloric acid (1M) and extracted with ethyl acetate (10 mL×2). The combined organic layers were dried over anhydrous sodium sulfate, filtered, and concentrated to afford 6-[4-(2-hydroxyethoxymethyl)-1-piperidyl]-2-[3-[3-[(4-methyl-1,2,4-triazol-3-yl)methyl]oxetan-3-yl]phenyl]-4-(trifluoromethyl)isoindolin-1-one (50 mg, 77%) as a light yellow solid. MS (ESI) m/z: 586.2 [M+H]⁺.

### Step 8: Preparation of 2-[[1-[2-[3-[3-[(4-methyl-1,2,4-triazol-3-yl)methyl]oxetan-3-yl]phenyl]-3-oxo-7-(trifluoromethyl)isoindolin-5-yl]-4-piperidyl]methoxy]ethyl 4-methylbenzenesulfonate

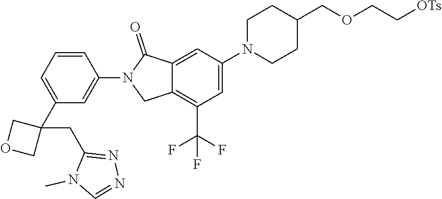 [SMILES: Cc1ccc(S(=O)(=O)OCCOCC2CCN(c3cc4c(c(C(F)(F)F)c3)CN(c3cccc(C5(Cc6nncn6C)COC5)c3)C4=O)CC2)cc1]

To a solution of 6-[4-(2-hydroxyethoxymethyl)-1-piperidyl]-2-[3-[3-[(4-methyl-1,2,4-triazol-3-yl)methyl]oxetan-3-yl]phenyl]-4-(trifluoromethyl)isoindolin-1-one (50 mg, 0.09 mmol), triethylamine (26 mg, 0.3 mmol) and 4-dimethylaminopyridine (10 mg, 0.09 mmol) in dichloromethane (5 mL) was added p-toluenesulfonyl chloride (33 mg, 0.18 mmol). The mixture was stirred at 25° C. for 2 h. The reaction mixture was quenched with brine (10 mL) and extracted with dichloromethane (10 mL×2). The combined organic layers were dried over anhydrous sodium sulfate, filtered, and concentrated. The residue was purified by preparative TLC (dichloromethane/methanol=10/1) to afford 2-[[1-[2-[3-[3-[(4-methyl-1,2,4-triazol-3-yl)methyl]oxetan-3-yl]phenyl]-3-oxo-7-(trifluoromethyl)isoindolin-5-yl]-4-piperidyl]methoxy]ethyl 4-methylbenzenesulfonate (68 mg, crude) as a light yellow solid. MS (ESI) m/z: 740.2 [M+H]⁺.

### Step 9: Preparation of 2-(2,6-dioxo-3-piperidyl)-5-[2-[[1-[2-[3-[3-[(4-methyl-1,2,4-triazol-3-yl)methyl]oxetan-3-yl]phenyl]-3-oxo-7-(trifluoromethyl)isoindolin-5-yl]-4-piperidyl]methoxy]ethoxy]isoindoline-1,3-dione

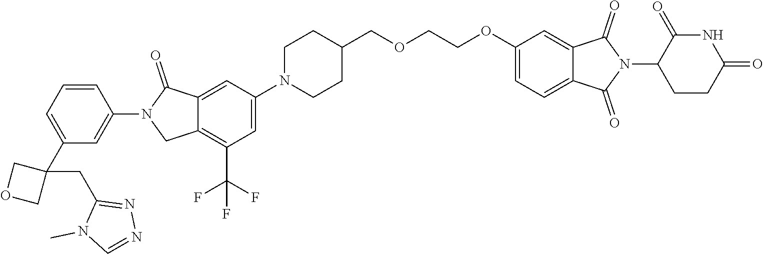 [SMILES: Cn1cnnc1CC1(c2cccc(N3Cc4c(cc(N5CCC(COCCOc6ccc7c(c6)C(=O)N(C6CCC(=O)NC6=O)C7=O)CC5)cc4C(F)(F)F)C3=O)c2)COC1]

To a solution of 2-[[1-[2-[3-[3-[(4-methyl-1,2,4-triazol-3-yl)methyl]oxetan-3-yl]phenyl]-3-oxo-7-(trifluoromethyl)isoindolin-5-yl]-4-piperidyl]methoxy]ethyl 4-methylbenzenesulfonate (68 mg, 0.09 mmol) and 2-(2,6-dioxo-3-piperidyl)-5-hydroxy-isoindoline-1,3-dione (38 mg, 0.1 mmol) in N,N-dimethylformamide (2 mL) was added potassium carbonate (38 mg, 0.3 mmol). The mixture was stirred at 50° C. for 10 h, then diluted with brine (10 mL) and extracted with dichloromethane (10 mL×2). The combined organic layers were dried over anhydrous sodium sulfate, filtered, and concentrated. The residue was purified by preparative HPLC (column: Phenomenex Synergi C18 150×25 mm×10 um; mobile phase: [water(FA)-ACN]; B %: 41%-59%, 9 min) to afford 2-(2,6-dioxo-3-piperidyl)-5-[2-[[1-[2-[3-[3-[(4-methyl-1,2,4-triazol-3-yl)methyl]oxetan-3-yl]phenyl]-3-oxo-7-(trifluoromethyl)isoindolin-5-yl]-4-piperidyl]methoxy]ethoxy]isoindoline-1,3-dione (18 mg, 22%) as an off-white solid. MS (ESI) m/z: 842.2 [M+H]⁺; ¹H NMR (400 MHz, DMSO-d₆) δ 11.17-11.03 (m, 1H), 8.18 (s, 1H), 7.90-7.86 (m, 1H), 7.83 (d, J=8.0 Hz, 1H), 7.48 (d, J=2.0 Hz, 2H), 7.43 (d, J=1.6 Hz, 1H), 7.38 (dd, J=2.4, 8.4 Hz, 1H), 7.34 (d, J=7.6 Hz, 2H), 6.74 (d, J=8.0 Hz, 1H), 5.12 (dd, J=5.4, 12.8 Hz, 1H), 4.97-4.94 (m, 4H), 4.88 (d, J=6.0 Hz, 2H), 4.37-4.32 (m, 2H), 3.89 (d, J=12.4 Hz, 2H), 3.80-3.74 (m, 2H), 3.50 (s, 2H), 3.38 (d, J=6.0 Hz, 2H), 2.89 (s, 3H), 2.81 (d, J=0.8 Hz, 2H), 2.61 (d, J=2.4 Hz, 1H), 2.59-2.55 (m, 2H), 2.07-2.01 (m, 1H), 1.78 (d, J=11.2 Hz, 3H), 1.32-1.23 (m, 2H).

### Example 16: Exemplary synthesis of 2-(2,6-dioxo-3-piperidyl)-5-[4-[[1-[[1-[[(3R)-1-[2-[3-[3-[(4-methyl-1,2,4-triazol-3-yl)methyl]oxetan-3-yl]phenyl]-3-oxo-7-(trifluoromethyl)isoindolin-5-yl]-3-piperidyl]methyl]-4-piperidyl]methyl]-4-piperidyl]methyl]piperazin-1-yl]isoindoline-1,3-dione

### Step 1: Preparation of [(3S)-3-piperidyl]methyl acetate

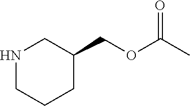

To a solution of tert-butyl (3S)-3-(acetoxymethyl)piperidine-1-carboxylate (1.80 g, 7 mmol) in dichloromethane (6 mL) was added trifluoroacetic acid (3.08 g, 27 mmol). The mixture was stirred at 25° C. for 1 h, then concentrated to afford [(3S)-3-piperidyl]methyl acetate trifluoroacetate (1.80 g, 95%) as a colorless oil. MS (ESI) m/z: 158.1 [M+H]⁺.

### Step 2: Preparation of [(3S)-1-[2-[3-[3-[(4-methyl-1,2,4-triazol-3-yl)methyl]oxetan-3-yl]phenyl]-3-oxo-7-(trifluoromethyl)isoindolin-5-yl]-3-piperidyl]methyl acetate

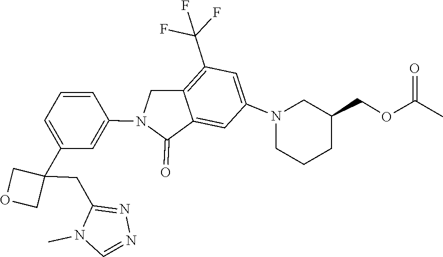 [SMILES: CC(=O)OC[C@H]1CCCN(c2cc3c(c(C(F)(F)F)c2)CN(c2cccc(C4(Cc5nncn5C)COC4)c2)C3=O)C1]

A mixture of 6-bromo-2-[3-[3-[(4-methyl-1,2,4-triazol-3-yl)methyl]oxetan-3-yl]phenyl]-4-(trifluoromethyl)isoindolin-1-one (400 mg, 0.8 mmol), [(3S)-3-piperidyl]methyl acetate trifluoroacetate (428 mg, 1.6 mmol), cesium carbonate (770 mg, 2.4 mmol) and XPhos Pd G4 (204 mg, 0.2 mmol) in dioxane (8 mL) was degassed and purged with nitrogen three times, then the mixture was stirred at 90° C. for 12 h under nitrogen. The reaction mixture was diluted with brine (20 mL) and extracted with dichloromethane (20 mL×2). The combined organic layers were dried over anhydrous sodium sulfate, filtered, and concentrated. The residue was purified by preparative TLC (dichloromethane/methanol=10/1) to afford [(3S)-1-[2-[3-[3-[(4-methyl-1,2,4-triazol-3-yl)methyl]oxetan-3-yl]phenyl]-3-oxo-7-(trifluoromethyl)isoindolin-5-yl]-3-piperidyl]methyl acetate (450 mg, 98%) as a light yellow solid. MS (ESI) m/z: 584.2 [M+H]⁺.

### Step 3: Preparation of 6-[(3S)-3-(hydroxymethyl)-1-piperidyl]-2-[3-[3-[(4-methyl-1,2,4-triazol-3-yl)methyl]oxetan-3-yl]phenyl]-4-(trifluoromethyl)isoindolin-1-one

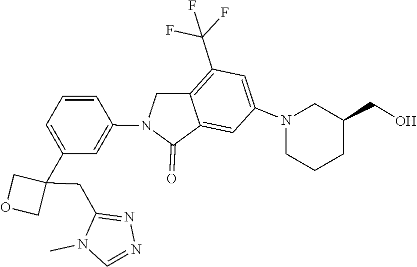 [SMILES: Cn1cnnc1CC1(c2cccc(N3Cc4c(cc(N5CCC[C@H](CO)C5)cc4C(F)(F)F)C3=O)c2)COC1]

To a solution of [(3S)-1-[2-[3-[3-[(4-methyl-1,2,4-triazol-3-yl)methyl]oxetan-3-yl]phenyl]-3-oxo-7-(trifluoromethyl)isoindolin-5-yl]-3-piperidyl]methyl acetate (450 mg, 0.8 mmol) in methanol (6 mL) and water (2 mL) was added sodium hydroxide (93 mg, 2.31 mmol). The mixture was stirred at 25° C. for 2 h, then diluted with brine (20 mL) and extracted with ethyl acetate (20 mL×2). The combined organic layers were collected and concentrated under reduced pressure to afford 6-[(3S)-3-(hydroxymethyl)-1-piperidyl]-2-[3-[3-[(4-methyl-1,2,4-triazol-3-yl)methyl]oxetan-3-yl]phenyl]-4-(trifluoromethyl)isoindolin-1-one (400 mg, 96%) as a light yellow solid.

### Step 4: Preparation of [(3S)-1-[2-[3-[3-[(4-methyl-1,2,4-triazol-3-yl)methyl]oxetan-3-yl]phenyl]-3-oxo-7-(trifluoromethyl)isoindolin-5-yl]-3-piperidyl]methyl 4-methylbenzenesulfonate

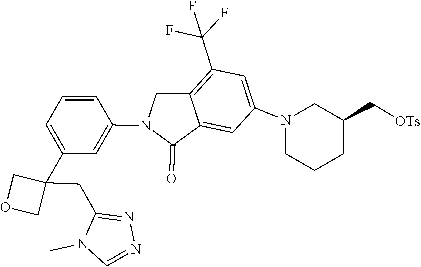 [SMILES: Cc1ccc(S(=O)(=O)OC[C@H]2CCCN(c3cc4c(c(C(F)(F)F)c3)CN(c3cccc(C5(Cc6nncn6C)COC5)c3)C4=O)C2)cc1]

To a solution of 6-[(3S)-3-(hydroxymethyl)-1-piperidyl]-2-[3-[3-[(4-methyl-1,2,4-triazol-3-yl)methyl]oxetan-3-yl]phenyl]-4-(trifluoromethyl)isoindolin-1-one (400 mg, 0.7 mmol), 4-dimethylaminopyridine (90 mg, 0.7 mmol) and triethylamine (224 mg, 2.2 mmol) in dichloromethane (5 mL) was added p-toluenesulfonyl chloride (282 mg, 1.5 mmol). The mixture was stirred at 25° C. for 1 h. The reaction mixture was diluted with brine (20 mL) and extracted with dichloromethane (20 mL×2). The combined organic layers were dried over anhydrous sodium sulfate, filtered, and concentrated. The residue was purified by preparative TLC (dichloromethane:methanol=10:1) to afford [(3S)-1-[2-[3-[3-[(4-methyl-1,2,4-triazol-3-yl)methyl]oxetan-3-yl]phenyl]-3-oxo-7-(trifluoromethyl)isoindolin-5-yl]-3-piperidyl]methyl 4-methylbenzenesulfonate (500 mg, 97%) as a light yellow solid. MS (ESI) m/z: 696.2 [M+H]⁺.

### Step 5: Preparation of 6-[(3R)-3-[[4-(dimethoxymethyl)-1-piperidyl]methyl]-1-piperidyl]-2-[3-[3-[(4-methyl-1,2,4-triazol-3-yl)methyl]oxetan-3-yl]phenyl]-4-(trifluoromethyl)isoindolin-1-one

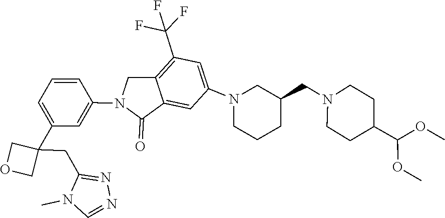 [SMILES: COC(OC)C1CCN(C[C@H]2CCCN(c3cc4c(c(C(F)(F)F)c3)CN(c3cccc(C5(Cc6nncn6C)COC5)c3)C4=O)C2)CC1]

To a solution of [(3S)-1-[2-[3-[3-[(4-methyl-1,2,4-triazol-3-yl)methyl]oxetan-3-yl]phenyl]-3-oxo-7-(trifluoromethyl)isoindolin-5-yl]-3-piperidyl]methyl 4-methylbenzenesulfonate (500 mg, 0.7 mmol) and 4-(dimethoxymethyl)piperidine (172 mg, 1.1 mmol) in dimethylsulfoxide (5 mL) was added diisopropylethylamine (279 mg, 2.2 mmol). The mixture was stirred at 100° C. for 10 h, then diluted with brine (20 mL) and extracted with dichloromethane (20 mL×2). The combined organic layers were dried over anhydrous sodium sulfate, filtered, and concentrated. The residue was purified by preparative TLC (dichloromethane:methanol=10:1) to afford 6-[(3R)-3-[[4-(dimethoxymethyl)-1-piperidyl]methyl]-1-piperidyl]-2-[3-[3-[(4-methyl-1,2,4-triazol-3-yl)methyl]oxetan-3-yl]phenyl]-4-(trifluoromethyl)isoindolin-1-one (300 mg, 61%) as a light yellow solid. MS (ESI) m/z: 683.4 [M+H]⁺.

### Step 6: Preparation of 1-[[(3R)-1-[2-[3-[3-[(4-methyl-1,2,4-triazol-3-yl)methyl]oxetan-3-yl]phenyl]-3-oxo-7-(trifluoromethyl)isoindolin-5-yl]-3-piperidyl]methyl]piperidine-4-carbaldehyde

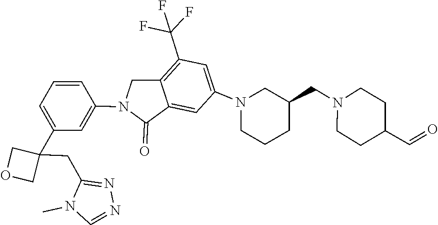 [SMILES: Cn1cnnc1CC1(c2cccc(N3Cc4c(cc(N5CCC[C@H](CN6CCC(C=O)CC6)C5)cc4C(F)(F)F)C3=O)c2)COC1]

To a solution of 6-[(3R)-3-[[4-(dimethoxymethyl)-1-piperidyl]methyl]-1-piperidyl]-2-[3-[3-[(4-methyl-1,2,4-triazol-3-yl)methyl]oxetan-3-yl]phenyl]-4-(trifluoromethyl)isoindolin-1-one (90 mg, 0.1 mmol) in dichloromethane (1 mL) was added trifluoroacetic acid (462 mg, 4.1 mmol). The mixture was stirred at 25° C. for 0.5 h, then concentrated to afford 1-[[(3R)-1-[2-[3-[3-[(4-methyl-1,2,4-triazol-3-yl)methyl]oxetan-3-yl]phenyl]-3-oxo-7-(trifluoromethyl)isoindolin-5-yl]-3-piperidyl]methyl]piperidine-4-carbaldehyde trifluoroacetate (90 mg, 91%) as a light yellow oil. MS (ESI) m/z: 637.2 [M+H]⁺.

### Step 7: Preparation of 2-(2,6-dioxo-3-piperidyl)-5-[4-[[1-[[1-[[(3R)-1-[2-[3-[3-[(4-methyl-1,2,4-triazol-3-yl)methyl]oxetan-3-yl]phenyl]-3-oxo-7-(trifluoromethyl)isoindolin-5-yl]-3-piperidyl]methyl]-4-piperidyl]methyl]-4-piperidyl]methyl]piperazin-1-yl]isoindoline-1,3-dione

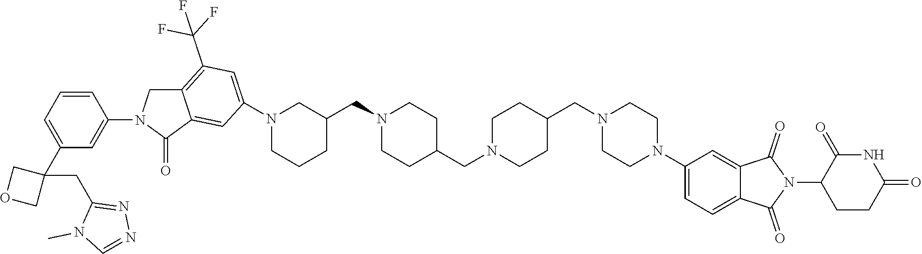

To a solution of 1-[[(3R)-1-[2-[3-[3-[(4-methyl-1,2,4-triazol-3-yl)methyl]oxetan-3-yl]phenyl]-3-oxo-7-(trifluoromethyl)isoindolin-5-yl]-3-piperidyl]methyl]piperidine-4-carbaldehyde trifluoroacetate (70 mg, 0.09 mmol) and 2-(2,6-dioxo-3-piperidyl)-5-[4-(4-piperidylmethyl)piperazin-1-yl]isoindoline-1,3-dione (61 mg, 0.1 mmol) in N,N-dimethylformamide (3 mL) and isopropanol (1 mL) was added triethylamine (28 mg, 0.3 mmol). The mixture was stirred at 25° C. for 10 h, then sodium cyanoborohydride (12 mg, 0.2 mmol) was added and stirred at 25° C. for 1 h. The reaction mixture was diluted with brine (20 mL) and extracted with dichloromethane (20 mL×2). The combined organic layers were dried over anhydrous sodium sulfate, filtered, and concentrated. The residue was purified by preparative HPLC (column: Phenomenex Luna C18 150×25 mm×10 um; mobile phase: [water(FA)-ACN]; B %: 2%-32%, 10 min) to afford 2-(2,6-dioxo-3-piperidyl)-5-[4-[[1-[[1-[[(3R)-1-[2-[3-[3-[(4-methyl-1,2,4-triazol-3-yl)methyl]oxetan-3-yl]phenyl]-3-oxo-7-(trifluoromethyl)isoindolin-5-yl]-3-piperidyl]methyl]-4-piperidyl]methyl]-4-piperidyl]methyl]piperazin-1-yl]isoindoline-1,3-dione (14 mg, 10%) as a yellow solid. MS (ESI) m/z: 1061.4 [M+H]⁺; ¹H NMR (400 MHz, DMSO-d₆) δ 11.09 (s, 1H), 8.20 (s, 1H), 7.88 (d, J=8.4 Hz, 1H), 7.68 (d, J=8.8 Hz, 1H), 7.43 (d, J=14.4 Hz, 2H), 7.38-7.31 (m, 3H), 7.26 (d, J=8.8 Hz, 1H), 6.77 (d, J=8.0 Hz, 1H), 5.08 (dd, J=5.2, 12.8 Hz, 1H), 5.01-4.93 (m, 4H), 4.89 (d, J=6.4 Hz, 2H), 3.77 (d, J=7.6 Hz, 2H), 3.51 (s, 3H), 3.43 (m, 5H), 3.03 (s, 2H), 2.96 (d, J=4.4 Hz, 2H), 2.91 (s, 3H), 2.89-2.79 (m, 2H), 2.76-2.69 (m, 1H), 2.63-2.56 (m, 1H), 2.44-2.35 (m, 4H), 2.18 (m, 4H), 2.07-1.90 (m, 4H), 1.82-1.67 (m, 7H), 1.66-1.51 (m, 4H), 1.31-1.12 (m, 6H).

### Example 17: Exemplary synthesis of 3-[4-fluoro-5-[4-[4-[2-[[2-[3-[3-[(4-methyl-1,2,4-triazol-3-yl)methyl]oxetan-3-yl]phenyl]-3-oxo-7-(trifluoromethyl)isoindolin-5-yl]amino]ethyl]cyclohexoxy]-1-piperidyl]-1-oxo-isoindolin-2-yl]piperidine-2,6-dione

### Step 1: Preparation of benzyl 4-[4-[(E)-2-methoxyvinyl]cyclohexoxy]piperidine-1-carboxylate

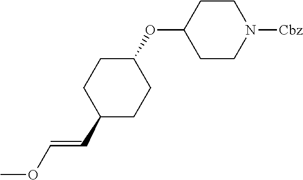 [SMILES: CO/C=C/[C@H]1CC[C@H](OC2CCN(C(=O)OCc3ccccc3)CC2)CC1]

To a solution of methoxymethyl(triphenyl)phosphonium chloride (3.97 g, 11.6 mmol) in tetrahydrofuran (30 mL) was added dropwise potassium tert-butoxide (1 M, 18 mL) at 0° C. under nitrogen. The resulting solution was stirred at 25° C. for 1 h. Then to the mixture was added dropwise a solution of benzyl 4-(4-formylcyclohexoxy)piperidine-1-carboxylate (2.00 g, 5.8 mmol) in tetrahydrofuran (10 mL). The mixture was stirred at 25° C. for 10 h, then used in next step without workup. Benzyl 4-[4-[(E)-2-methoxyvinyl]cyclohexoxy]piperidine-1-carboxylate (2.10 g, crude) was obtained as yellow oil.

### Step 2: Preparation of benzyl 4-[4-(2-oxoethyl)cyclohexoxy]piperidine-1-carboxylate

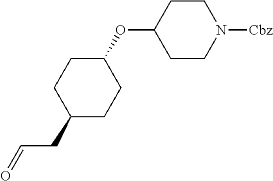 [SMILES: O=CC[C@H]1CC[C@H](OC2CCN(C(=O)OCc3ccccc3)CC2)CC1]

To a solution of benzyl 4-[4-[(E)-2-methoxyvinyl]cyclohexoxy]piperidine-1-carboxylate (2.10 g, 5.6 mmol) in tetrahydrofuran (20 mL) was added 6 M hydrochloric acid solution (42 mL) at 0° C. The mixture was stirred at 25° C. for 0.5 h. The mixture was diluted with saturated sodium bicarbonate (50 mL) and extracted with dichloromethane (50 mL×2). The organic layer was concentrated. The residue was purified by column chromatography (petroleum ether/ethyl acetate=10/1 to 4/1) and then further purified by preparative HPLC (column: Phenomenex Luna C18 150×40 mm×15 um; mobile phase: [water(FA)-ACN]; B %: 50%-80%, 10 min) to give benzyl 4-[4-(2-oxoethyl)cyclohexoxy]piperidine-1-carboxylate (760 mg, 38%) as an off white oil. MS (ESI) m/z: 360.2[M+H]⁺; ¹H NMR (400 MHz, CDCl₃) δ 0.95-1.14 (m, 2H) 1.24-1.39 (m, 3H) 1.48-1.51 (m, 2H) 1.79-1.87 (m, 4H) 1.95-2.02 (m, 2H) 2.29-2.36 (m, 2H) 3.13-3.24 (m, 2H) 3.27-3.33 (m, 1H) 3.53-3.62 (m, 1H) 3.82-3.93 (m, 2H) 5.14 (s, 2H) 7.34-7.40 (m, 4H) 9.78 (dt, J=6.4, 2.0 Hz, 1H).

### Step 3: Preparation of benzyl 4-(((1r, 4r)-4-(2,2-dimethoxyethyl)cyclohexyl)oxy)piperidine-1-carboxylate

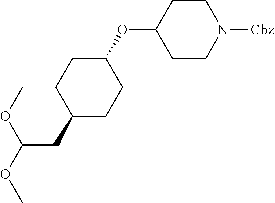 [SMILES: COC(C[C@H]1CC[C@H](OC2CCN(C(=O)OCc3ccccc3)CC2)CC1)OC]

To a solution of benzyl 4-[4-(2-oxoethyl)cyclohexoxy]piperidine-1-carboxylate (740 mg, 2.1 mmol) in methanol (10 mL) was added 4-methylbenzenesulfonic acid (35 mg, 0.2 mmol) and trimethoxymethane (437 mg, 4.1 mmol). The mixture was stirred at 25° C. for 1 h, diluted with saturated sodium bicarbonate (20 mL) and concentrated. The mixture was extracted with dichloromethane (10 mL×2). The organic layer was concentrated to afford benzyl 4-(((1r, 4r)-4-(2,2-dimethoxyethyl)cyclohexyl)oxy)piperidine-1-carboxylate (740 mg, 89%) as an off white oil. MS (ESI) m/z: 428.3[M+Na]⁺; ¹H NMR (400 MHz, CDCl₃) δ 0.82-0.96 (m, 2H) 1.11-1.22 (m, 2H) 1.26-1.34 (m, 1H) 1.38-1.50 (m, 4H) 1.64-1.80 (m, 4H) 1.87 (br d, J=10.2 Hz, 2H) 3.09 (ddd, J=13.2, 9.2, 3.6 Hz, 2H) 3.14-3.21 (m, 1H) 3.23 (s, 6H) 3.51 (dt, J=8.0, 4.4 Hz, 1H) 3.70-3.87 (m, 2H) 4.38 (t, J=6.0 Hz, 1H) 5.05 (s, 2H) 7.21-7.35 (m, 4H).

### Step 4: Preparation of 4-[4-(2,2-dimethoxyethyl)cyclohexoxy]piperidine

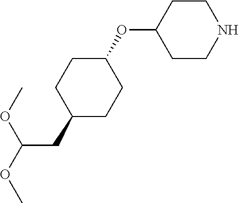 [SMILES: COC(C[C@H]1CC[C@H](OC2CCNCC2)CC1)OC]

To a solution of benzyl 4-[4-(2,2-dimethoxyethyl)cyclohexoxy]piperidine-1-carboxylate (740 mg, 1.8 mmol) in methanol (10 mL) was added 10% palladium on activated carbon (200 mg). Then the mixture was stirred at 25° C. for 10 h under hydrogen atmosphere. The mixture was filtered through Celite and the filtrate was concentrated under reduced pressure to give 4-[4-(2,2-dimethoxyethyl)cyclohexoxy]piperidine (460 mg, 93%) as an off white oil.

### Step 5: Preparation of 3-[5-[4-[4-(2,2-dimethoxyethyl)cyclohexoxy]-1-piperidyl]-4-fluoro-1-oxo-isoindolin-2-yl]piperidine-2,6-dione

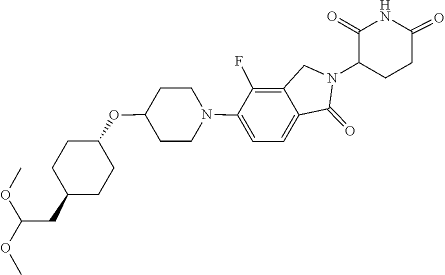 [SMILES: COC(C[C@H]1CC[C@H](OC2CCN(c3ccc4c(c3F)CN(C3CCC(=O)NC3=O)C4=O)CC2)CC1)OC]

To a solution of 4-[4-(2,2-dimethoxyethyl)cyclohexoxy]piperidine (430 mg, 1.6 mmol) and 3-(4,5-difluoro-1-oxo-isoindolin-2-yl)piperidine-2,6-dione (444 mg, 1.6 mmol) in dimethylsulfoxide (5 mL) was added diisopropylethylamine (614 mg, 4.8 mmol). The mixture was stirred at 120° C. for 10 h. The mixture was suspended in water (50 mL) and filtered. The solid was triturated with petroleum ether:ethyl acetate=8:1 (10 mL) at 25° C. for 20 min, then filtered to afford 3-[5-[4-[4-(2,2-dimethoxyethyl)cyclohexoxy]-1-piperidyl]-4-fluoro-1-oxo-isoindolin-2-yl]piperidine-2,6-dione (520 mg, 62%) as a brown solid. MS (ESI) m/z: 532.3 [M+H]⁺.

### Step 6: Preparation of 2-[4-[[1-[2-(2,6-dioxo-3-piperidyl)-4-fluoro-1-oxo-isoindolin-5-yl]-4-piperidyl]oxy]cyclohexyl]acetaldehyde

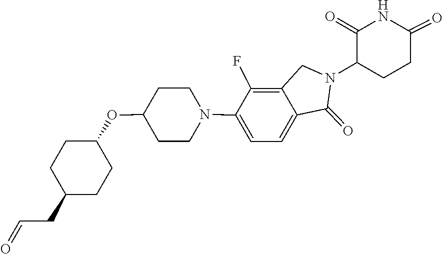 [SMILES: O=CC[C@H]1CC[C@H](OC2CCN(c3ccc4c(c3F)CN(C3CCC(=O)NC3=O)C4=O)CC2)CC1]

To a solution of 3-[5-[4-[4-(2,2-dimethoxyethyl)cyclohexoxy]-1-piperidyl]-4-fluoro-1-oxo-isoindolin-2-yl]piperidine-2,6-dione (100 mg, 0.2 mmol) in dichloromethane (2 mL) was added trifluoroacetic acid (2 mL, 27 mmol). The mixture was stirred at 25° C. for 0.5 h, then concentrated under reduced pressure to afford 2-[4-[[1-[2-(2,6-dioxo-3-piperidyl)-4-fluoro-1-oxo-isoindolin-5-yl]-4-piperidyl]oxy]cyclohexyl]acetaldehyde (90 mg, 98%) as a brown oil. MS (ESI) m/z: 486.3[M+H]⁺.

### Step 7: Preparation of tert-butyl N-[2-[3-[3-[(4-methyl-1,2,4-triazol-3-yl)methyl]oxetan-3-yl]phenyl]-3-oxo-7-(trifluoromethyl)isoindolin-5-yl]carbamate

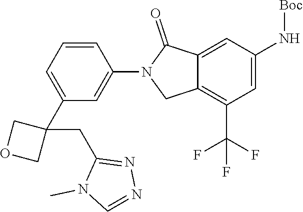 [SMILES: Cn1cnnc1CC1(c2cccc(N3Cc4c(cc(NC(=O)OC(C)(C)C)cc4C(F)(F)F)C3=O)c2)COC1]

A solution of 6-bromo-2-[3-[3-[(4-methyl-1,2,4-triazol-3-yl)methyl]oxetan-3-yl]phenyl]-4-(trifluoromethyl)isoindolin-1-one (200 mg, 0.4 mmol) tert-butyl carbamate (60 mg, 0.5 mmol), cesium carbonate (385 mg, 1.2 mmol) and Xphos Pd G4 (34 mg, 0.04 mmol) in dioxane (4 mL) was purged by nitrogen three times. Then the mixture was stirred at 90° C. for 4 h under nitrogen atmosphere, then concentrated. The residue was purified by column chromatography (petroleum ether/ethyl acetate=1/1 to dichloromethane: methanol=20/1) to give tert-butyl N-[2-[3-[3-[(4-methyl-1,2,4-triazol-3-yl)methyl]oxetan-3-yl]phenyl]-3-oxo-7-(trifluoromethyl)isoindolin-5-yl]carbamate (150 mg, 70%) as a yellow oil. MS (ESI) m/z: 544.4[M+H]⁺.

### Step 8: Preparation of 6-amino-2-[3-[3-[(4-methyl-1,2,4-triazol-3-yl)methyl]oxetan-3-yl]phenyl]-4-(trifluoromethyl)isoindolin-1-one

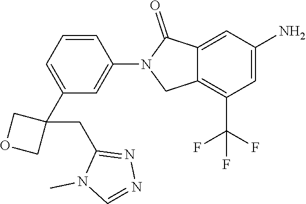 [SMILES: Cn1cnnc1CC1(c2cccc(N3Cc4c(cc(N)cc4C(F)(F)F)C3=O)c2)COC1]

To a solution of tert-butyl N-[2-[3-[3-[(4-methyl-1,2,4-triazol-3-yl)methyl]oxetan-3-yl]phenyl]-3-oxo-7-(trifluoromethyl)isoindolin-5-yl]carbamate (150 mg, 0.3 mmol) in dichloromethane (2 mL) was added trifluoroacetic acid (1 mL). The mixture was stirred at 25° C. for 0.5 h, then concentrated. The residue was purified by preparative HPLC (column: Phenomenex Luna C18 150×25 mm×10 um; mobile phase: [water(FA)-ACN]; B %: 22%-49%, 9 min) to give 6-amino-2-[3-[3-[(4-methyl-1,2,4-triazol-3-yl)methyl]oxetan-3-yl]phenyl]-4-(trifluoromethyl)isoindolin-1-one as a white solid. MS (ESI) m/z: 444.1[M+H]⁺; ¹H NMR (400 MHz, DMSO-d₆) δ 2.89 (s, 3H) 3.51 (s, 2H) 4.89 (d, J=6.0 Hz, 4H) 4.96 (d, J=6.0 Hz, 2H) 5.91-5.94 (m, 1H) 6.72 (d, J=8.0 Hz, 1H) 7.16 (d, J=1.6 Hz, 2H) 7.28-7.44 (m, 2H) 7.85 (dd, J=8.4, 1.4 Hz, 1H) 8.19 (s, 1H).

### Step 9: Preparation of 3-[4-fluoro-5-[4-[4-[2-[[2-[3-[3-[(4-methyl-1,2,4-triazol-3-yl)methyl]oxetan-3-yl]phenyl]-3-oxo-7-(trifluoromethyl)isoindolin-5-yl]amino]ethyl]cyclohexoxy]-1-piperidyl]-1-oxo-isoindolin-2-yl]piperidine-2,6-dione

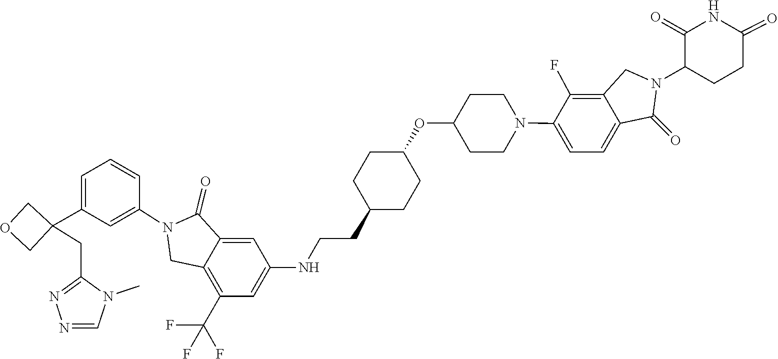 [SMILES: Cn1cnnc1CC1(c2cccc(N3Cc4c(cc(NCC[C@H]5CC[C@H](OC6CCN(c7ccc8c(c7F)CN(C7CCC(=O)NC7=O)C8=O)CC6)CC5)cc4C(F)(F)F)C3=O)c2)COC1]

A solution of 6-amino-2-[3-[3-[(4-methyl-1,2,4-triazol-3-yl)methyl]oxetan-3-yl]phenyl]-4-(trifluoromethyl)isoindolin-1-one trifluoroacetate (80 mg, 0.1 mmol), 2-[4-[[1-[2-(2,6-dioxo-3-piperidyl)-4-fluoro-1-oxo-isoindolin-5-yl]-4-piperidyl]oxy]cyclohexyl]acetaldehyde (84 mg, 0.2 μmol), triethylamine (15 mg, 0.1 μmol) and chloro(triisopropoxy)titanium (37 mg, 0.1 mmol) in dichloromethane (2 mL) was stirred at 25° C. for 10 h. Then sodium triacetoxyborohydride (91 mg, 0.4 mmol) was added and the mixture was stirred at 25° C. for 1 h. The reaction was diluted with water (10 mL) and extracted with ethyl acetate (10 mL). The organic layer was concentrated. The residue was purified by preparative HPLC (column: Phenomenex Luna C18 150×25 mm×10 um; mobile phase: [water(FA)-ACN]; B %: 50%-80%, 10 min) to give 3-[4-fluoro-5-[4-[4-[2-[[2-[3-[3-[(4-methyl-1,2,4-triazol-3-yl)methyl]oxetan-3-yl]phenyl]-3-oxo-7-(trifluoromethyl)isoindolin-5-yl]amino]ethyl]cyclohexoxy]-1-piperidyl]-1-oxo-isoindolin-2-yl]piperidine-2,6-dione (20 mg, 14%) as a white solid. MS (ESI) m/z: 913.2 [M+H]⁺; ¹H NMR (400 MHz, DMSO-d₆) δ 0.96-1.07 (m, 2H) 1.11-1.23 (m, 3H) 1.35-1.43 (m, 1H) 1.46-1.52 (m, 2H) 1.54-1.61 (m, 2H) 1.77-1.84 (m, 2H) 1.92 (br d, J=2.0 Hz, 2H) 1.94-2.02 (m, 4H) 2.43 (br s, 1H) 2.57 (br s, 1H) 2.89 (s, 2H) 2.91-2.96 (m, 2H) 3.11-3.15 (m, 1H) 3.30 (s, 3H) 3.37 (br d, J=2.8 Hz, 2H) 3.51 (s, 2H) 3.60-3.67 (m, 1H) 4.31 (d, J=16.8 Hz, 1H) 4.48 (d, J=17.2 Hz, 1H) 4.87-4.91 (m, 4H) 4.96 (d, J=6.0 Hz, 2H) 5.08 (dd, J=13.2, 4.8 Hz, 1H) 6.43 (t, J=4.8 Hz, 1H) 6.73 (d, J=6.8 Hz, 1H) 7.07 (s, 1H) 7.14-7.20 (m, 2H) 7.29-7.40 (m, 2H) 7.47 (d, J=8.0 Hz, 1H) 7.80-7.98 (m, 1H) 8.19 (s, 1H) 10.98 (s, 1H).

### Example 18: Exemplary synthesis of 2-cyclopropyl-6-[7-[[1-[2-(2,6-dioxo-3-piperidyl)-1,3-dioxo-isoindolin-5-yl]-4-piperidyl]methyl]-2,7-diazaspiro[3.5]nonan-2-yl]-N-[3-[3-[(4-methyl-1,2,4-triazol-3-yl)methyl]oxetan-3-yl]phenyl]pyrimidine-4-carboxamide

### Step 1: Preparation of 6-chloro-2-cyclopropyl-pyrimidine-4-carboxylic acid

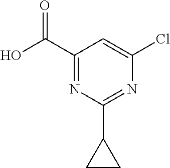 [SMILES: O=C(O)c1cc(Cl)nc(C2CC2)n1]

To 2-cyclopropyl-6-hydroxy-pyrimidine-4-carboxylic acid (1 g, 5.6 mmol) was added phosphorus oxychloride (4 mL). The mixture was stirred at 90° C. for 12 h, then cooled to room temperature, then added to ice water (30 mL). The resulting mixture was extracted with ethyl acetate (20 mL×3). The combined organic layers were washed brine (30 mL×3), dried over sodium sulfate, filtered, and concentrated. The residue was purified by flash silica gel chromatography (0-10% methanol/dichloromethane) to afford 6-chloro-2-cyclopropyl-pyrimidine-4-carboxylic acid (750 mg, 68%) as a pink solid. MS (ESI) m/z: 198.8 [M+H]⁺; ¹H NMR (400 MHz, CDCl₃) δ 14.34-13.79 (m, 1H), 7.80 (s, 1H), 2.31-2.24 (m, 1H), 1.18-1.12 (m, 2H), 1.08-1.04 (m, 2H).

### Step 2: Preparation of methyl 6-chloro-2-cyclopropyl-pyrimidine-4-carboxylate

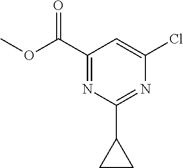

To a solution of 6-chloro-2-cyclopropyl-pyrimidine-4-carboxylic acid (500 mg, 2.5 mmol) in tetrahydrofuran (10 mL) was added trimethylsilyl diazomethane (2.0 M, 1.9 mL). The mixture was stirred at 20° C. for 1 h. Water (20 mL) was poured into the mixture and stirred for 1 min, then the aqueous phase was extracted with ethyl acetate (20 mL×3). The combined organic phase was washed with brine (20 mL×2), dried with anhydrous sodium sulfate, filtered, and concentrated under vacuum. The residue was purified by flash silica gel chromatography (0-30% ethyl acetate/petroleum ether) to afford methyl 6-chloro-2-cyclopropyl-pyrimidine-4-carboxylate (330 mg, 61%) as a white solid. MS (ESI) m/z: 212.9 [M+H]⁺.

### Step 3: Preparation of tert-butyl 2-(2-cyclopropyl-6-methoxycarbonyl-pyrimidin-4-yl)-2,7-diazaspiro[3.5]nonane-7-carboxylate

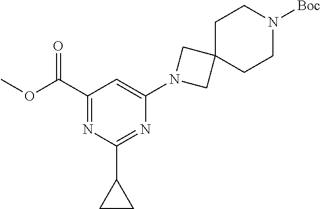 [SMILES: COC(=O)c1cc(N2CC3(CCN(C(=O)OC(C)(C)C)CC3)C2)nc(C2CC2)n1]

To a solution of methyl 6-chloro-2-cyclopropyl-pyrimidine-4-carboxylate (250 mg, 1.2 mmol) and tert-butyl 2,7-diazaspiro[3.5]nonane-7-carboxylate (292 mg, 1.29 mmol) in acetonitrile (5 mL) was added diisopropylethylamine (1.02 mL, 5.9 mmol). The mixture was stirred at 80° C. for 2 h. Water (20 mL) was poured into the mixture and stirred for 1 min, then the aqueous phase was extracted with ethyl acetate (20 mL×3). The combined organic phase was washed with brine (20 mL×2), dried with anhydrous sodium sulfate, filtered, and concentrated under vacuum. The residue was purified by flash silica gel chromatography (0-30% ethyl acetate/petroleum ether) to afford tert-butyl 2-(2-cyclopropyl-6-methoxycarbonyl-pyrimidin-4-yl)-2,7-diazaspiro[3.5]nonane-7-carboxylate (450 mg, 95%) as a white solid. MS (ESI) m/z: 403.4 [M+H]⁺.

### Step 4: Preparation of 6-(7-tert-butoxycarbonyl-2,7-diazaspiro[3.5]nonan-2-yl)-2-cyclopropyl-pyrimidine-4-carboxylic acid

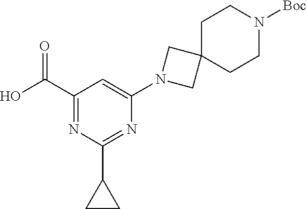 [SMILES: CC(C)(C)OC(=O)N1CCC2(CC1)CN(c1cc(C(=O)O)nc(C3CC3)n1)C2]

To a solution of tert-butyl 2-(2-cyclopropyl-6-methoxycarbonyl-pyrimidin-4-yl)-2,7-diazaspiro[3.5]nonane-7-carboxylate (450 mg, 1.1 mmol) in ethyl alcohol (7 mL), water (7 mL) and tetrahydrofuran (7 mL) was added lithium hydroxide (160 mg, 6.7 mmol). The mixture was stirred at 40° C. for 2 h. The pH of the reaction mixture was adjusted to 5 with 1 M sulfuric acid solution. Water (100 mL) was added to the mixture and the mixture was extracted with ethyl acetate (100 mL×3). The combined organic phase was washed with brine (100 mL×2), dried with anhydrous sodium sulfate, filtered, and concentrated under vacuum to afford 6-(7-tert-butoxycarbonyl-2,7-diazaspiro[3.5]nonan-2-yl)-2-cyclopropyl-pyrimidine-4-carboxylic acid (430 mg, 99%) as a yellow solid. MS (ESI) m/z: 389.3 [M+H]⁺.

### Step 5: Preparation of tert-butyl 2-[2-cyclopropyl-6-[[3-[3-[(4-methyl-1,2,4-triazol-3-yl)methyl]oxetan-3-yl]phenyl]carbamoyl]pyrimidin-4-yl]-2,7-diazaspiro[3.5]nonane-7-carboxylate

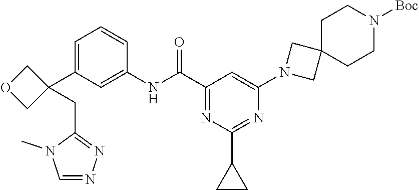 [SMILES: Cn1cnnc1CC1(c2cccc(NC(=O)c3cc(N4CC5(CCN(C(=O)OC(C)(C)C)CC5)C4)nc(C4CC4)n3)c2)COC1]

To a solution of 6-(7-tert-butoxycarbonyl-2,7-diazaspiro[3.5]nonan-2-yl)-2-cyclopropyl-pyrimidine-4-carboxylic acid (200 mg, 515 μmol) in N,N-dimethylformamide (3 mL) was added triethylamine (0.08 mL, 0.6 mmol), hydroxybenzotriazole (800 mg, 6 mmol) and 1-(3-dimethylaminopropyl)-3-ethylcarbodiimide hydrochloride (296 mg, 1.5 mmol). The mixture was stirred at 25° C. for 0.5 h, then 3-[3-[(4-methyl-1,2,4-triazol-3-yl)methyl]oxetan-3-yl]aniline (125 mg, 0.5 mmol) was added and the mixture was stirred 25° C. for 12 h. Water (10 mL) was poured into the mixture, and the aqueous layer was extracted with ethyl acetate (10 mL×3). The combined organic phase was washed with brine (10 mL×2), dried over anhydrous sodium sulfate, filtered, and concentrated under vacuum. The residue was purified by flash silica gel chromatography (0-5% methanol/dichloromethane) to afford tert-butyl 2-[2-cyclopropyl-6-[[3-[3-[(4-methyl-1,2,4-triazol-3-yl)methyl]oxetan-3-yl]phenyl]carbamoyl]pyrimidin-4-yl]-2,7-diazaspiro[3.5]nonane-7-carboxylate (260 mg, 82%) as a yellow solid. MS (ESI) m/z: 615.2 [M+H]⁺.

### Step 6: Preparation of 2-cyclopropyl-6-(2,7-diazaspiro[3.5]nonan-2-yl)-N-[3-[3-[(4-methyl-1,2,4-triazol-3-yl)methyl]oxetan-3-yl]phenyl]pyrimidine-4-carboxamide

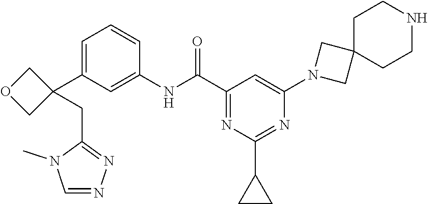 [SMILES: Cn1cnnc1CC1(c2cccc(NC(=O)c3cc(N4CC5(CCNCC5)C4)nc(C4CC4)n3)c2)COC1]

To a solution of tert-butyl 2-[2-cyclopropyl-6-[[3-[3-[(4-methyl-1,2,4-triazol-3-yl)methyl]oxetan-3-yl]phenyl]carbamoyl]pyrimidin-4-yl]-2,7-diazaspiro[3.5]nonane-7-carboxylate (100 mg, 0.2 mmol) in dichloromethane (1 mL) was added trifluoroacetic acid (0.01 mL, 0.2 mmol). The mixture was stirred at 20° C. for 0.5 h, then concentrated under reduced pressure to afford 2-cyclopropyl-6-(2,7-diazaspiro[3.5]nonan-2-yl)-N-[3-[3-[(4-methyl-1,2,4-triazol-3-yl)methyl]oxetan-3-yl]phenyl]pyrimidine-4-carboxamide (83 mg, 99%) as a yellow oil. MS (ESI) m/z: 515.3 [M+H]⁺.

### Step 7: Preparation of 2-cyclopropyl-6-[7-[[1-[2-(2,6-dioxo-3-piperidyl)-1,3-dioxo-isoindolin-5-yl]-4-piperidyl]methyl]-2,7-diazaspiro[3.5]nonan-2-yl]-N-[3-[3-[(4-methyl-1,2,4-triazol-3-yl)methyl]oxetan-3-yl]phenyl]pyrimidine-4-carboxamide

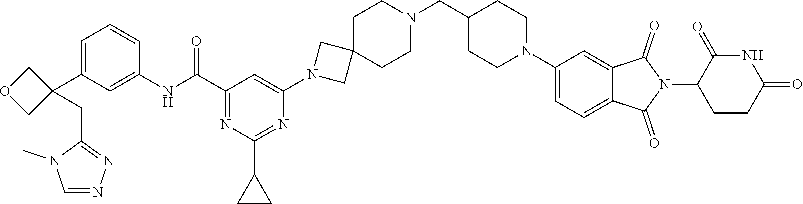 [SMILES: Cn1cnnc1CC1(c2cccc(NC(=O)c3cc(N4CC5(CCN(CC6CCN(c7ccc8c(c7)C(=O)N(C7CCC(=O)NC7=O)C8=O)CC6)CC5)C4)nc(C4CC4)n3)c2)COC1]

To a solution of 2-cyclopropyl-6-(2,7-diazaspiro[3.5]nonan-2-yl)-N-[3-[3-[(4-methyl-1,2,4-triazol-3-yl)methyl]oxetan-3-yl]phenyl]pyrimidine-4-carboxamide (83 mg, 0.2 mmol) in dichloromethane (1.5 mL) was added triethylamine (0.06 mL, 0.5 mmol). The mixture was stirred at 20° C. for 10 min, then 1-[2-(2,6-dioxo-3-piperidyl)-1,3-dioxo-isoindolin-5-yl]piperidine-4-carbaldehyde (59 mg, 0.2 mmol) was added and the mixture was stirred for 20 min. Lastly, sodium triacetoxyborohydride (102 mg, 0.5 mmol) was added, and the mixture was stirred at 20° C. for 12 h. The reaction mixture was concentrated and the resulting residue was purified by preparative HPLC (column: Phenomenex Luna C18 150×25 mm×10 um; mobile phase: [water(FA)-ACN]; B %: 9%-39%) to afford 2-cyclopropyl-6-[7-[[1-[2-(2,6-dioxo-3-piperidyl)-1,3-dioxo-isoindolin-5-yl]-4-piperidyl]methyl]-2,7-diazaspiro[3.5]nonan-2-yl]-N-[3-[3-[(4-methyl-1,2,4-triazol-3-yl)methyl]oxetan-3-yl]phenyl]pyrimidine-4-carboxamide formate (106.1 mg, 69%) as a yellow solid. MS (ESI) m/z: 867.42 [M+H]⁺; ¹H NMR (400 MHz, CDCl₃) δ 11.07 (s, 1H), 10.22 (s, 1H), 8.19 (s, 1H), 8.13 (s, 1H), 7.81-7.75 (m, 1H), 7.66 (d, J=8.4 Hz, 1H), 7.44 (s, 1H), 7.32 (s, 1H), 7.30-7.21 (m, 2H), 6.73 (s, 1H), 6.67 (d, J=7.6 Hz, 1H), 5.06 (dd, J=5.4, 12.8 Hz, 1H), 4.93 (d, J=6.0 Hz, 2H), 4.85 (d, J=6.0 Hz, 2H), 4.06 (d, J=12.8 Hz, 2H), 3.80 (s, 4H), 3.48 (s, 2H), 3.02-2.94 (m, 2H), 2.92 (s, 3H), 2.90-2.83 (m, 1H), 2.58 (d, J=16.0 Hz, 2H), 2.54 (d, J=5.6 Hz, 6H), 2.15-2.07 (m, 1H), 2.06-1.97 (m, 1H), 1.94-1.74 (m, 7H), 1.25-1.12 (m, 2H), 1.07 (d, J=4.0 Hz, 2H), 1.01-0.92 (m, 2H).

### Example 19: Exemplary synthesis of 2-cyclopropyl-6-[[(3R)-3-[[4-[[4-[2-(2,6-dioxo-3-piperidyl)-1,3-dioxo-isoindolin-5-yl]piperazin-1-yl]methyl]-1-piperidyl]methyl]-1-piperidyl]methyl]-N-[3-[3-[(4-methyl-1,2,4-triazol-3-yl)methyl]oxetan-3-yl]phenyl]pyrimidine-4-carboxamide

### Step 1: Preparation of methyl 2-cyclopropyl-6-vinyl-pyrimidine-4-carboxylate

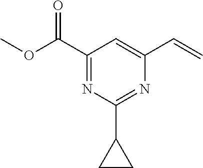

To a solution of methyl 6-chloro-2-cyclopropyl-pyrimidine-4-carboxylate (1.0 g, 4.7 mmol) and potassium trifluoro(vinyl)boranuide (1.26 g, 9.4 mmol) in dioxane (15 mL) and water (3 mL) was added [1,1′-bis(diphenylphosphino)ferrocene]dichloropalladium(II) (344 mg, 0.5 mmol) and potassium carbonate (1.95 g, 14 mmol). The mixture was stirred at 90° C. for 12 h. Water (50 mL) was poured into the mixture, and the aqueous layer was extracted with ethyl acetate (50 mL×3). The combined organic phase was washed with brine (50 mL×2), dried over anhydrous sodium sulfate, filtered, and concentrated under vacuum. The residue was purified by flash silica gel chromatography (0-10% ethyl acetate/petroleum ether) to afford methyl 2-cyclopropyl-6-vinyl-pyrimidine-4-carboxylate (740 mg, 77%) as a white solid. MS (ESI) m/z: 205.0 [M+H]⁺.

### Step 2: Preparation of methyl 2-cyclopropyl-6-formyl-pyrimidine-4-carboxylate

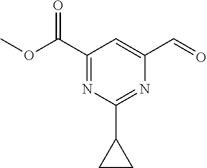

To a solution of methyl 2-cyclopropyl-6-vinyl-pyrimidine-4-carboxylate (440 mg, 2.2 mmol) in dioxane (10 mL) and water (2 mL) was added sodium periodate (477.54 μL, 8.6 mmol), potassium osmate(VI) dihydrate (158 mg, 0.4 mmol) and 2,6-dimethylpyridine (752.8 μL, 6.5 mmol). The mixture was stirred at 20° C. for 12 h. Water (80 mL) was poured into the mixture, and the aqueous layer was extracted with ethyl acetate (20 mL×3). The combined organic phase was washed with brine (30 mL×2), dried over anhydrous sodium sulfate, filtered, and concentrated under vacuum. The residue was purified by column chromatography (petroleum ether/ethyl acetate=50/1 to 1/1) to afford methyl 2-cyclopropyl-6-formyl-pyrimidine-4-carboxylate (160 mg, 36%) as a colorless oil. MS (ESI) m/z: 224.9 [M+H₂O+H]⁺.

### Step 3: Preparation of methyl 2-cyclopropyl-6-(dimethoxymethyl)pyrimidine-4-carboxylate

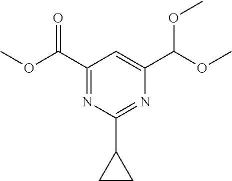

To a solution of methyl 2-cyclopropyl-6-formyl-pyrimidine-4-carboxylate (160 mg, 0.8 mmol) in methanol (1 mL) was added p-toluenesulfonic acid (26 mg, 0.2 mmol) and trimethoxymethane (851 μL, 7.8 mmol). The mixture was stirred at 20° C. for 12 h. Water (30 mL) was poured into the mixture and the aqueous layer was extracted with ethyl acetate (10 mL×3). The combined organic phase was washed with brine (10 mL×2), dried over anhydrous sodium sulfate, filtered, and concentrated under vacuum. The residue was purified by column chromatography (petroleum ether/ethyl acetate=30/1 to 1/1) to afford methyl 2-cyclopropyl-6-(dimethoxymethyl)pyrimidine-4-carboxylate (190 mg, 97%) as a white solid. MS (ESI) m/z: 253.1 [M+H]⁺.

### Step 4: Preparation of 2-cyclopropyl-6-(dimethoxymethyl)pyrimidine-4-carboxylic acid

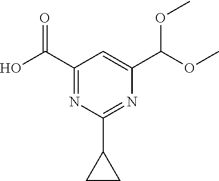 [SMILES: COC(OC)c1cc(C(=O)O)nc(C2CC2)n1]

To a solution of methyl 2-cyclopropyl-6-(dimethoxymethyl)pyrimidine-4-carboxylate (190 mg, 0.8 mmol) in methanol (2 mL), tetrahydrofuran (2 mL) and water (2 mL) was added lithium hydroxide monohydrate (126 mg, 3 mmol). The mixture was stirred at 25° C. for 1 h. The pH of the reaction mixture was adjusted to 6 with 1M hydrochloric acid solution. The aqueous phase was extracted with ethyl acetate (10 mL×3). The combined organic phase was washed with brine (10 mL), dried over anhydrous sodium sulfate, filtered, and concentrated to afford 2-cyclopropyl-6-(dimethoxymethyl)pyrimidine-4-carboxylic acid (150 mg, crude) as a white solid. MS (ESI) m/z: 239.0 [M+H]⁺.

### Step 5: Preparation of 2-cyclopropyl-6-(dimethoxymethyl)-N-[3-[3-[(4-methyl-1,2,4-triazol-3-yl)methyl]oxetan-3-yl]phenyl]pyrimidine-4-carboxamide

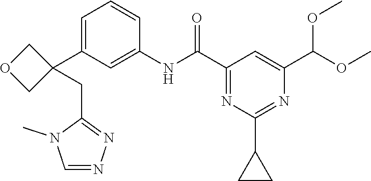 [SMILES: COC(OC)c1cc(C(=O)Nc2cccc(C3(Cc4nncn4C)COC3)c2)nc(C2CC2)n1]

To a solution of 2-cyclopropyl-6-(dimethoxymethyl)pyrimidine-4-carboxylic acid (150 mg, 0.6 mmol) and 3-[3-[(4-methyl-1,2,4-triazol-3-yl)methyl]oxetan-3-yl]aniline (153 mg, 0.6 mmol) in dimethyl formamide (3 mL) was added triethylamine (175 μL, 1.26 mmol) and hydroxybenzotriazole (127 mg, 0.9 mmol). The mixture was stirred at 20° C. for 0.5 h. 1-(3-dimethylaminopropyl)-3-ethylcarbodiimide hydrochloride (241 mg, 1.3 mmol) was added to the mixture, and stirred at 20° C. for 11.5 h. Water (50 mL) was poured into the mixture and it was extracted with ethyl acetate (20 mL×3). The combined organic phase was washed with brine (20 mL×2), dried over anhydrous sodium sulfate, filtered, and concentrated under vacuum. The residue was purified by column chromatography (dichloromethane:methanol=1:0 to 10:1) to afford 2-cyclopropyl-6-(dimethoxymethyl)-N-[3-[3-[(4-methyl-1,2,4-triazol-3-yl)methyl]oxetan-3-yl]phenyl]pyrimidine-4-carboxamide (250 mg, 85%) as a white solid. MS (ESI) m/z: 465.2 [M+H]⁺.

### Step 6: Preparation of 2-cyclopropyl-6-formyl-N-[3-[3-[(4-methyl-1,2,4-triazol-3-yl)methyl]oxetan-3-yl]phenyl]pyrimidine-4-carboxamide

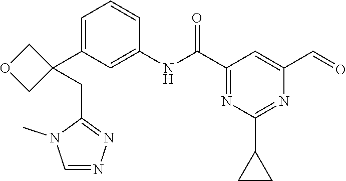 [SMILES: Cn1cnnc1CC1(c2cccc(NC(=O)c3cc(C=O)nc(C4CC4)n3)c2)COC1]

To a solution of 2-cyclopropyl-6-(dimethoxymethyl)-N-[3-[3-[(4-methyl-1,2,4-triazol-3-yl)methyl]oxetan-3-yl]phenyl]pyrimidine-4-carboxamide (60 mg, 0.1 mmol) in tetrahydrofuran (1.5 mL) was added 2 M sulfuric acid (1.3 mL). The mixture was stirred at 70° C. for 0.5 h. The pH of the reaction mixture was adjusted to 7 with saturated aqueous sodium bicarbonate solution. The aqueous phase was extracted with ethyl acetate (10 mL×3). The combined organic phase was washed with brine (10 mL×2), dried over anhydrous sodium sulfate, filtered, and concentrated under vacuum. The residue was purified by preparative TLC (dichloromethane:methanol=10:1) to afford 2-cyclopropyl-6-formyl-N-[3-[3-[(4-methyl-1,2,4-triazol-3-yl)methyl]oxetan-3-yl]phenyl]pyrimidine-4-carboxamide (50 mg, 92%) as a white solid. MS (ESI) m/z: 436.9 [M+H]⁺.

### Step 7: Preparation of 2-cyclopropyl-6-[[(3R)-3-[[4-[[4-[2-(2,6-dioxo-3-piperidyl)-1,3-dioxo-isoindolin-5-yl]piperazin-1-yl]methyl]-1-piperidyl]methyl]-1-piperidyl]methyl]-N-[3-[3-[(4-methyl-1,2,4-triazol-3-yl)methyl]oxetan-3-yl]phenyl]pyrimidine-4-carboxamide

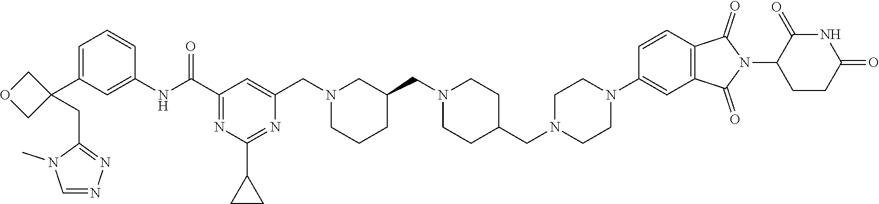 [SMILES: Cn1cnnc1CC1(c2cccc(NC(=O)c3cc(CN4CCC[C@H](CN5CCC(CN6CCN(c7ccc8c(c7)C(=O)N(C7CCC(=O)NC7=O)C8=O)CC6)CC5)C4)nc(C4CC4)n3)c2)COC1]

To a solution of 2-cyclopropyl-6-formyl-N-[3-[3-[(4-methyl-1,2,4-triazol-3-yl)methyl]oxetan-3-yl]phenyl]pyrimidine-4-carboxamide (50 mg, 0.11 mmol) and 2-(2,6-dioxo-3-piperidyl)-5-[4-[[1-[[(3S)-3-piperidyl]methyl]-4-piperidyl]methyl]piperazin-1-yl]isoindoline-1,3-dione hydrochloride (68 mg, 0.1 mmol,) in dichloromethane (5 mL) was added triethylamine (33.26 μL, 0.2 mmol). The mixture was stirred at 20° C. for 0.5 h. Sodium triacetoxyborohydride (75 mg, 0.4 mmol) was added to the mixture, stirred at 20° C. for 11.5 h, then concentrated under reduced pressure. The residue was purified by preparative HPLC (column: Welch Xtimate C18 150×25 mm×5 um; mobile phase: [water(FA)-ACN]; B %: 5%-35%, 10 min) to afford 2-cyclopropyl-6-[[(3R)-3-[[4-[[4-[2-(2,6-dioxo-3-piperidyl)-1,3-dioxo-isoindolin-5-yl]piperazin-1-yl]methyl]-1-piperidyl]methyl]-1-piperidyl]methyl]-N-[3-[3-[(4-methyl-1,2,4-triazol-3-yl)methyl]oxetan-3-yl]phenyl]pyrimidine-4-carboxamide (9.2 mg, 8%) as a yellow solid. MS (ESI) m/z: 939.7 [M+H]⁺; ¹H NMR (400 MHz, DMSO-d₆) δ 11.08 (s, 1H), 10.43 (s, 1H), 8.20 (s, 1H), 8.13 (s, 1H), 7.87 (s, 1H), 7.77 (d, J=6.8 Hz, 1H), 7.68 (d, J=8.4 Hz, 1H), 7.49 (s, 1H), 7.33 (s, 1H), 7.31-7.21 (m, 2H), 6.76-6.69 (m, 1H), 5.10-5.04 (m, 1H), 4.93 (d, J=6.0 Hz, 2H), 4.85 (d, J=6.0 Hz, 2H), 3.65 (d, J=2.4 Hz, 2H), 3.49 (s, 2H), 3.40 (s, 6H), 3.30 (s, 8H), 2.94 (s, 3H), 2.91-2.83 (m, 2H), 2.82-2.75 (m, 1H), 2.62-2.58 (m, 2H), 2.41-2.34 (m, 2H), 2.23-2.10 (m, 4H), 2.07-1.95 (m, 3H), 1.87-1.62 (m, 6H), 1.60-1.52 (m, 1H), 1.19-1.11 (m, 4H).

### Example 20: Exemplary synthesis of 2-(2,6-dioxo-3-piperidyl)-5-[2-[2-[2-[3-[3-[(4-methyl-1,2,4-triazol-3-yl)methyl]oxetan-3-yl]phenyl]-3-oxo-7-(trifluoromethyl)isoindolin-5-yl]-2-azaspiro[3.3]heptan-6-yl]ethoxy]isoindoline-1,3-dione

### Step 1: Preparation of tert-butyl 6-(2-acetoxyethyl)-2-azaspiro[3.3]heptane-2-carboxylate

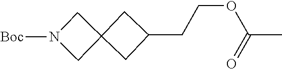 [SMILES: CC(=O)OCCC1CC2(C1)CN(C(=O)OC(C)(C)C)C2]

To a solution of tert-butyl 6-(2-hydroxyethyl)-2-azaspiro[3.3]heptane-2-carboxylate (500 mg, 2.1 mmol) and triethylamine (419 mg, 4.14 mmol) in dichloromethane (5 mL) was added acetyl chloride (244 mg, 3.11 mmol). The mixture was stirred at 25° C. for 2 h. The reaction mixture was quenched with water (10 mL) and extracted with ethyl acetate (10 mL×2). The combined organic phase was washed with brine (20 mL), dried over anhydrous sodium sulfate, filtered, and concentrated. The residue was purified by column chromatography (petroleum ether/ethyl acetate=20/1 to 5/1) to afford tert-butyl 6-(2-acetoxyethyl)-2-azaspiro[3.3]heptane-2-carboxylate (0.52 g, 89%) as colorless oil. MS (ESI) m/z: 184.2 [M-100+H]⁺; ¹H NMR (400 MHz, CDCl₃) δ 4.03-3.98 (m, 2H), 3.96-3.92 (m, 2H), 3.81 (s, 2H), 2.33-2.27 (m, 2H), 2.25-2.17 (m, 1H), 2.05 (s, 3H), 1.86-1.79 (m, 2H), 1.70 (q, J=6.8 Hz, 2H), 1.44 (s, 9H).

### Step 2: Preparation of 2-(2-azaspiro[3.3]heptan-6-yl)ethyl acetate

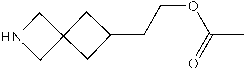

To a solution of tert-butyl 6-(2-acetoxyethyl)-2-azaspiro[3.3]heptane-2-carboxylate (0.52 g, 1.8 mmol) in dichloromethane (5 mL) was added trifluoroacetic acid (1.93 g, 17 mmol). The mixture was stirred at 25° C. for 2 h, then concentrated to afford 2-(2-azaspiro[3.3]heptan-6-yl)ethyl acetate trifluoroacetate (500 mg, 92%) as a colorless oil. MS (ESI) m/z: 184.2 [M+H]⁺; ¹H NMR (400 MHz, DMSO-d₆) δ 4.00-3.95 (m, 2H), 3.92 (t, J=6.4 Hz, 2H), 3.85 (t, J=6.4 Hz, 2H), 2.34-2.27 (m, 2H), 2.17-2.09 (m, 1H), 1.98 (s, 3H), 1.89-1.80 (m, 2H), 1.67-1.57 (m, 2H).

### Step 3: Preparation of 2-[2-[2-[3-[3-[(4-methyl-1,2,4-triazol-3-yl)methyl]oxetan-3-yl]phenyl]-3-oxo-7-(trifluoromethyl)isoindolin-5-yl]-2-azaspiro[3.3]heptan-6-yl]ethyl acetate

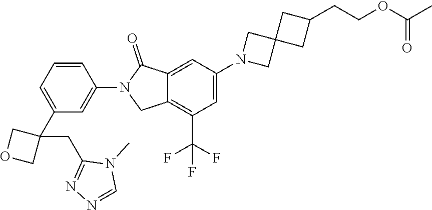 [SMILES: CC(=O)OCCC1CC2(C1)CN(c1cc3c(c(C(F)(F)F)c1)CN(c1cccc(C4(Cc5nncn5C)COC4)c1)C3=O)C2]

A mixture of 6-bromo-2-[3-[3-[(4-methyl-1,2,4-triazol-3-yl)methyl]oxetan-3-yl]phenyl]-4-(trifluoromethyl)isoindolin-1-one (50 mg, 0.1 mmol), 2-(2-azaspiro[3.3]heptan-6-yl)ethyl acetate trifluoroacetate (58 mg, 0.2 mmol), cesium carbonate (96 mg, 0.3 mmol) and XPhos Pd G4 (25 mg, 0.03 mmol) in dioxane (3 mL) was degassed and purged with nitrogen three times, then the mixture was stirred at 90° C. for 12 h under nitrogen. The reaction mixture was diluted with brine (20 mL) and extracted with dichloromethane (20 mL×2). The combined organic layers were dried over anhydrous sodium sulfate, filtered, and concentrated. The residue was purified by preparative TLC (dichloromethane/methanol=10/1) to afford 2-[2-[2-[3-[3-[(4-methyl-1,2,4-triazol-3-yl)methyl]oxetan-3-yl]phenyl]-3-oxo-7-(trifluoromethyl)isoindolin-5-yl]-2-azaspiro[3.3]heptan-6-yl]ethyl acetate (50 mg, 83%) as a white solid. MS (ESI) m/z: 610.1 [M+H]⁺.

### Step 4: Preparation of 6-[6-(2-hydroxyethyl)-2-azaspiro[3.3]heptan-2-yl]-2-[3-[3-[(4-methyl-1,2,4-triazol-3-yl)methyl]oxetan-3-yl]phenyl]-4-(trifluoromethyl)isoindolin-1-one

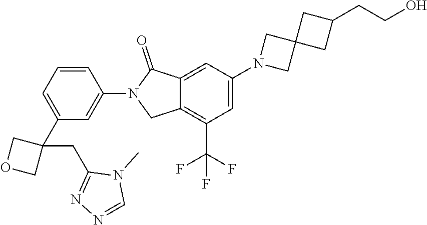 [SMILES: Cn1cnnc1CC1(c2cccc(N3Cc4c(cc(N5CC6(CC(CCO)C6)C5)cc4C(F)(F)F)C3=O)c2)COC1]

To a solution of 2-[2-[2-[3-[3-[(4-methyl-1,2,4-triazol-3-yl)methyl]oxetan-3-yl]phenyl]-3-oxo-7-(trifluoromethyl)isoindolin-5-yl]-2-azaspiro[3.3]heptan-6-yl]ethyl acetate (50 mg, 0.08 mmol) in methanol (6 mL) and water (2 mL) was added sodium hydroxide (10 mg, 0.2 mmol). The mixture was stirred at 25° C. for 2 h, diluted with brine (20 mL) and extracted with dichloromethane (20 mL×2). The combined organic layers were dried over anhydrous sodium sulfate, filtered, and concentrated. The residue was purified by preparative TLC (dichloromethane/methanol=10/1) to afford 6-[6-(2-hydroxyethyl)-2-azaspiro[3.3]heptan-2-yl]-2-[3-[3-[(4-methyl-1,2,4-triazol-3-yl)methyl]oxetan-3-yl]phenyl]-4-(trifluoromethyl)isoindolin-1-one (40 mg, 86%) as a white solid. MS (ESI) m/z: 568.2 [M+H]⁺.

### Step 5: Preparation of 2-[2-[2-[3-[3-[(4-methyl-1,2,4-triazol-3-yl)methyl]oxetan-3-yl]phenyl]-3-oxo-7-(trifluoromethyl)isoindolin-5-yl]-2-azaspiro[3.3]heptan-6-yl]ethyl 4-methylbenzenesulfonate

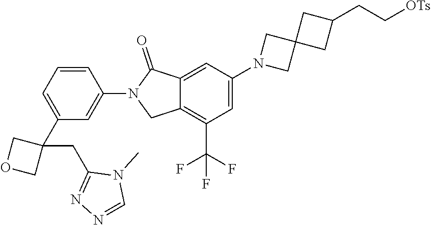 [SMILES: Cc1ccc(S(=O)(=O)OCCC2CC3(C2)CN(c2cc4c(c(C(F)(F)F)c2)CN(c2cccc(C5(Cc6nncn6C)COC5)c2)C4=O)C3)cc1]

To a solution of 6-[6-(2-hydroxyethyl)-2-azaspiro[3.3]heptan-2-yl]-2-[3-[3-[(4-methyl-1,2,4-triazol-3-yl)methyl]oxetan-3-yl]phenyl]-4-(trifluoromethyl)isoindolin-1-one (50 mg, 0.09 mmol) in dichloromethane (5 mL) was added triethylamine (27 mg, 0.3 mmol) and p-toluenesulfonyl chloride (34 mg, 0.2 mmol). The mixture was stirred at 25° C. for 3 h, then quenched with brine (10 mL) and extracted with dichloromethane (10 mL×2). The combined organic layers were dried over anhydrous sodium sulfate, filtered, and concentrated. The residue was purified by preparative TLC (dichloromethane/methanol=10/1) to afford 2-[2-[2-[3-[3-[(4-methyl-1,2,4-triazol-3-yl)methyl]oxetan-3-yl]phenyl]-3-oxo-7-(trifluoromethyl)isoindolin-5-yl]-2-azaspiro[3.3]heptan-6-yl]ethyl 4-methylbenzenesulfonate (55 mg, 87%) as a light yellow solid. MS (ESI) m/z: 722.2 [M+H]⁺.

### Step 6: Preparation of 2-(2,6-dioxo-3-piperidyl)-5-[2-[2-[2-[3-[3-[(4-methyl-1,2,4-triazol-3-yl)methyl]oxetan-3-yl]phenyl]-3-oxo-7-(trifluoromethyl)isoindolin-5-yl]-2-azaspiro[3.3]heptan-6-yl]ethoxy]isoindoline-1,3-dione

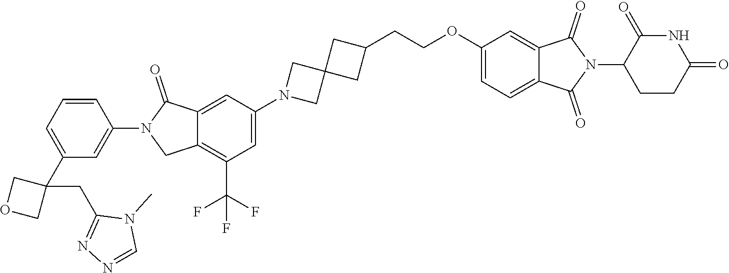 [SMILES: Cn1cnnc1CC1(c2cccc(N3Cc4c(cc(N5CC6(CC(CCOc7ccc8c(c7)C(=O)N(C7CCC(=O)NC7=O)C8=O)C6)C5)cc4C(F)(F)F)C3=O)c2)COC1]

To a solution of 2-[2-[2-[3-[3-[(4-methyl-1,2,4-triazol-3-yl)methyl]oxetan-3-yl]phenyl]-3-oxo-7-(trifluoromethyl)isoindolin-5-yl]-2-azaspiro[3.3]heptan-6-yl]ethyl 4-methylbenzenesulfonate (50 mg, 0.07 mmol) and 2-(2,6-dioxo-3-piperidyl)-5-hydroxy-isoindoline-1,3-dione (23 mg, 0.08 mmol) in N,N-dimethylformamide (2 mL) was added potassium carbonate (29 mg, 0.2 mmol). The mixture was stirred at 50° C. for 10 h, then diluted with brine (10 mL) and extracted with dichloromethane (10 mL×2). The combined organic layers were dried over anhydrous sodium sulfate, filtered, and concentrated. The residue was purified by preparative HPLC (column: Phenomenex Synergi C18 150×25 mm×10 um; mobile phase: [water(FA)-ACN]; B %: 45%-72%, 9 min) to afford 2-(2,6-dioxo-3-piperidyl)-5-[2-[2-[2-[3-[3-[(4-methyl-1,2,4-triazol-3-yl)methyl]oxetan-3-yl]phenyl]-3-oxo-7-(trifluoromethyl)isoindolin-5-yl]-2-azaspiro[3.3]heptan-6-yl]ethoxy]isoindoline-1,3-dione (21 mg, 36%) as a white solid. MS (ESI) m/z: 824.3 [M+H]⁺; ¹H NMR (400 MHz, DMSO-d₆) δ 11.12 (s, 1H), 8.19 (s, 1H), 7.93-7.76 (m, 2H), 7.43 (d, J=2.0 Hz, 1H), 7.40-7.31 (m, 3H), 6.91 (d, J=7.2 Hz, 2H), 6.75 (d, J=7.6 Hz, 1H), 5.13 (dd, J=5.2, 12.8 Hz, 1H), 4.99-4.92 (m, 4H), 4.89 (d, J=6.0 Hz, 2H), 4.16 (t, J=6.2 Hz, 2H), 4.00 (s, 2H), 3.89 (s, 2H), 3.51 (s, 2H), 2.90 (s, 3H), 2.62 (d, J=2.4 Hz, 1H), 2.55 (s, 2H), 2.42-2.35 (m, 3H), 2.08 (s, 1H), 1.98 (d, J=4.4 Hz, 2H), 1.89 (d, J=6.0 Hz, 2H).

### Example 21: Exemplary synthesis of 3-[5-[4-[[1-[2-[3-[3-[(4-methyl-1,2,4-triazol-3-yl)methyl]oxetan-3-yl]phenyl]-3-oxo-7-(trifluoromethyl)isoindolin-5-yl]azetidin-3-yl]oxymethyl]-1-piperidyl]-1-oxo-isoindolin-2-yl]piperidine-2,6-dione

### Step 1: Preparation of tert-butyl 4-(p-tolylsulfonyloxymethyl)piperidine-1-carboxylate

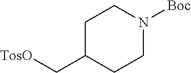

To a solution of tert-butyl 4-(hydroxymethyl)piperidine-1-carboxylate (3.2 g, 14.9 mmol) and triethylamine (4.51 g, 44.6 mmol) in dichloromethane (16 mL) was added p-toluenesulfonyl chloride (4.25 g, 22.3 mmol) at 0° C. The reaction mixture was stirred at 20° C. for 16 h, diluted with water (60 mL) and extracted with ethyl acetate (60 mL×2). The combined organic layer was dried over anhydrous sodium sulfate, filtered, and concentrated. The residue was purified by silica gel column chromatography (petroleum ether/ethyl acetate=100/1 to 8:1) to afford tert-butyl 4-(p-tolylsulfonyloxymethyl)piperidine-1-carboxylate (4.82 g, 88%) as a light yellow gum.

### Step 2: Preparation of tert-butyl 4-[(1-benzyloxycarbonylazetidin-3-yl)oxymethyl]piperidine-1-carboxylate

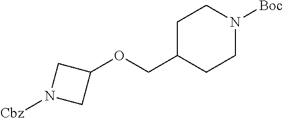 [SMILES: CC(C)(C)OC(=O)N1CCC(COC2CN(C(=O)OCc3ccccc3)C2)CC1]

To a solution of benzyl 3-hydroxyazetidine-1-carboxylate (3.28 g, 15.8 mmol) in N,N-dimethylformamide (30 mL) was added 60% sodium hydride (569 mg, 14.2 mmol) at 0° C. The mixture was stirred at 0° C. for 0.5 h, then tert-butyl 4-(p-tolylsulfonyloxymethyl)piperidine-1-carboxylate (2.92 g, 7.9 mmol) in N,N-dimethylformamide (15 mL) was added to the mixture at 0° C. and the reaction mixture was stirred at 50° C. for 12 h. Saturated ammonium chloride solution (100 mL) was added and the resulting mixture was extracted with ethyl acetate (70 mL×2). The combined organic layer was washed with brine (40 mL×3), dried over anhydrous sodium sulfate, filtered, and concentrated. The residue was purified by silica gel column chromatography (petroleum ether/ethyl acetate=10/1 to 2:1) to afford tert-butyl 4-[(1-benzyloxycarbonylazetidin-3-yl)oxymethyl]piperidine-1-carboxylate (2.27 g, 71%) as a light yellow gum. ¹H NMR (400 MHz, CDCl₃) δ 7.28-7.42 (m, 5H), 5.10 (s, 2H), 4.20-4.27 (m, 1H), 4.04-4.19 (m, 4H), 3.89 (dd, J=10.0, 4.0 Hz, 2H), 3.19 (d, J=5.6 Hz, 2H), 2.70 (s, 2H), 1.71 (dd, J=11.6, 2.4 Hz, 3H), 1.46 (s, 9H), 1.14 (dd, J=12.4, 3.6 Hz, 2H).

### Step 3: Preparation of tert-butyl 4-(azetidin-3-yloxymethyl)piperidine-1-carboxylate

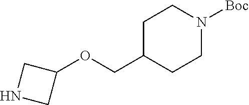 [SMILES: CC(C)(C)OC(=O)N1CCC(COC2CNC2)CC1]

To a solution of tert-butyl 4-[(1-benzyloxycarbonylazetidin-3-yl)oxymethyl]piperidine-1-carboxylate (870 mg, 2.2 mmol) in tetrahydrofuran (10 mL) and tetrafluoroethylene (15 mL) was added 10% palladium on activated carbon (100 mg) and 20% palladium hydroxide (100 mg) under nitrogen. The mixture was degassed and purged with hydrogen three times. The reaction mixture was stirred at 20° C. under hydrogen (50 Psi) for 12 h. The reaction mixture was filtered with a pad of Celite and the filtrate was concentrated to afford tert-butyl 4-(azetidin-3-yloxymethyl)piperidine-1-carboxylate (570 mg, 98%) as a light yellow gum.

### Step 4: Preparation of tert-butyl 4-[[1-[2-[3-[3-[(4-methyl-1,2,4-triazol-3-yl)methyl]oxetan-3-yl]phenyl]-3-oxo-7-(trifluoromethyl)isoindolin-5-yl]azetidin-3-yl]oxymethyl]piperidine-1-carboxylate

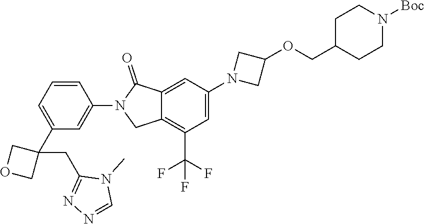 [SMILES: Cn1cnnc1CC1(c2cccc(N3Cc4c(cc(N5CC(OCC6CCN(C(=O)OC(C)(C)C)CC6)C5)cc4C(F)(F)F)C3=O)c2)COC1]

A mixture of 6-bromo-2-[3-[3-[(4-methyl-1,2,4-triazol-3-yl)methyl]oxetan-3-yl]phenyl]-4-(trifluoromethyl)isoindolin-1-one (50 mg, 0.1 mmol), tert-butyl 4-(azetidin-3-yloxymethyl)piperidine-1-carboxylate (53.29 mg, 197 μmol), cesium carbonate (96 mg, 0.29 mmol) and XPhos Pd G4 (17 mg, 0.02 mmol) in dioxane (2.5 mL) was degassed and purged with nitrogen three times, then the mixture was stirred at 90° C. for 12 h under nitrogen atmosphere. The mixture was filtered and concentrated. The residue was purified by preparative TLC (dichloromethane:methanol=10:1) to afford tert-butyl 4-[[1-[2-[3-[3-[(4-methyl-1,2,4-triazol-3-yl)methyl]oxetan-3-yl]phenyl]-3-oxo-7-(trifluoromethyl)isoindolin-5-yl]azetidin-3-yl]oxymethyl]piperidine-1-carboxylate (50 mg, 62%) as a yellow oil. MS (ESI) m/z: 697.3 [M+H]⁺.

### Step 5: Preparation of 2-[3-[3-[(4-methyl-1,2,4-triazol-3-yl)methyl]oxetan-3-yl]phenyl]-6-[3-(4-piperidylmethoxy)azetidin-1-yl]-4-(trifluoromethyl)isoindolin-1-one

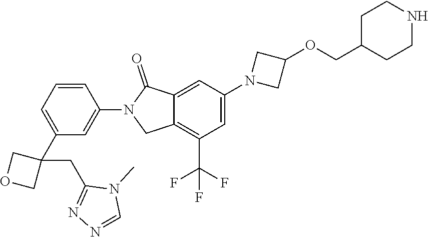 [SMILES: Cn1cnnc1CC1(c2cccc(N3Cc4c(cc(N5CC(OCC6CCNCC6)C5)cc4C(F)(F)F)C3=O)c2)COC1]

To a solution of tert-butyl 4-[[1-[2-[3-[3-[(4-methyl-1,2,4-triazol-3-yl)methyl]oxetan-3-yl]phenyl]-3-oxo-7-(trifluoromethyl)isoindolin-5-yl]azetidin-3-yl]oxymethyl]piperidine-1-carboxylate (50 mg, 0.07 mmol) in dichloromethane (1.5 mL) was added trifluoroacetic acid (770 mg, 6.7 mmol). The mixture was stirred at 25° C. for 0.5 h, then concentrated under reduced pressure to afford 2-[3-[3-[(4-methyl-1,2,4-triazol-3-yl)methyl]oxetan-3-yl]phenyl]-6-[3-(4-piperidylmethoxy)azetidin-1-yl]-4-(trifluoromethyl)isoindolin-1-one trifluoroacetate (48 mg, 94%) as a yellow oil. MS (ESI) m/z: 597.2 [M+H]⁺.

### Step 6: Preparation of 2-(2,6-dioxo-3-piperidyl)-5-[4-[[1-[2-[3-[3-[(4-methyl-1,2,4-triazol-3-yl)methyl]oxetan-3-yl]phenyl]-3-oxo-7-(trifluoromethyl)isoindolin-5-yl]azetidin-3-yl]methyl]-1-piperidyl]isoindoline-1,3-dione

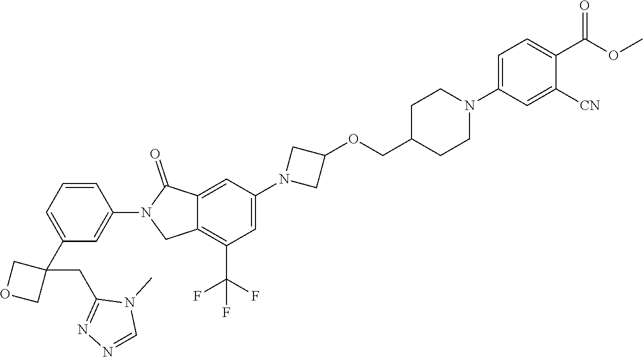 [SMILES: COC(=O)c1ccc(N2CCC(COC3CN(c4cc5c(c(C(F)(F)F)c4)CN(c4cccc(C6(Cc7nncn7C)COC6)c4)C5=O)C3)CC2)cc1C#N]

To a solution of 2-[3-[3-[(4-methyl-1,2,4-triazol-3-yl)methyl]oxetan-3-yl]phenyl]-6-[3-(4-piperidylmethoxy)azetidin-1-yl]-4-(trifluoromethyl)isoindolin-1-one trifluoroacetate (200 mg, 0.3 mmol) and methyl 2-cyano-4-fluoro-benzoate (76 mg, 0.4 mmol) in dimethylsulfoxide (4 mL) was added diisopropylethylamine (109 mg, 0.8 mmol). The mixture was stirred at 100° C. for 10 h, then diluted with brine (20 mL) and extracted with dichloromethane (20 mL×2). The combined organic layers were dried over anhydrous sodium sulfate, filtered, and concentrated. The residue was purified by preparative TLC (dichloromethane/methanol=10/1) to afford methyl 2-cyano-4-[4-[[1-[2-[3-[3-[(4-methyl-1,2,4-triazol-3-yl)methyl]oxetan-3-yl]phenyl]-3-oxo-7-(trifluoromethyl)isoindolin-5-yl]azetidin-3-yl]oxymethyl]-1-piperidyl]benzoate (90 mg, 42%) as a light yellow solid. MS (ESI) m/z: 756.2 [M+H]⁺.

### Step 7: Preparation of methyl 2-formyl-4-[4-[[1-[2-[3-[3-[(4-methyl-1,2,4-triazol-3-yl)methyl]oxetan-3-yl]phenyl]-3-oxo-7-(trifluoromethyl)isoindolin-5-yl]azetidin-3-yl]oxymethyl]-1-piperidyl]benzoate

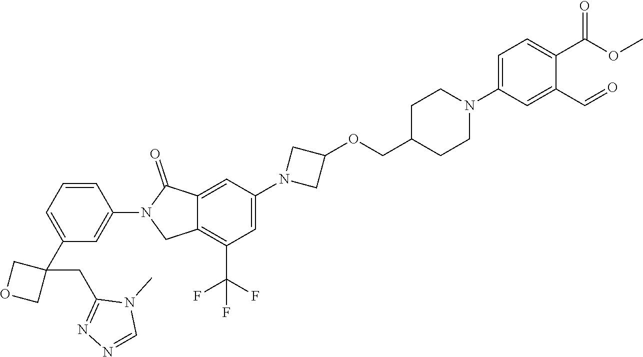 [SMILES: COC(=O)c1ccc(N2CCC(COC3CN(c4cc5c(c(C(F)(F)F)c4)CN(c4cccc(C6(Cc7nncn7C)COC6)c4)C5=O)C3)CC2)cc1C=O]

To a solution of methyl 2-cyano-4-[4-[[1-[2-[3-[3-[(4-methyl-1,2,4-triazol-3-yl)methyl]oxetan-3-yl]phenyl]-3-oxo-7-(trifluoromethyl)isoindolin-5-yl]azetidin-3-yl]oxymethyl]-1-piperidyl]benzoate (80 mg, 0.1 mmol) in pyridine (4 mL), acetic acid (4 mL) and water (2 mL) was added Raney-Ni reagent (9 mg, 0.1 mmol) and sodium dihydrogenphosphate hydrate (146 mg, 1.1 mmol). The mixture was stirred at 60° C. for 2 h, quenched with brine (40 mL) and filtered. The filtrate was washed with 5% citric acid solution (30 mL×3) and dried over anhydrous sodium sulfate, filtered, and concentrated to afford methyl 2-formyl-4-[4-[[1-[2-[3-[3-[(4-methyl-1,2,4-triazol-3-yl)methyl]oxetan-3-yl]phenyl]-3-oxo-7-(trifluoromethyl)isoindolin-5-yl]azetidin-3-yl]oxymethyl]-1-piperidyl]benzoate (75 mg, 93%) as a colorless solid. MS (ESI) m/z: 759.3 [M+H]⁺.

### Step 8: Preparation of 3-[5-[4-[[1-[2-[3-[3-[(4-methyl-1,2,4-triazol-3-yl)methyl]oxetan-3-yl]phenyl]-3-oxo-7-(trifluoromethyl)isoindolin-5-yl]azetidin-3-yl]oxymethyl]-1-piperidyl]-1-oxo-isoindolin-2-yl]piperidine-2,6-dione

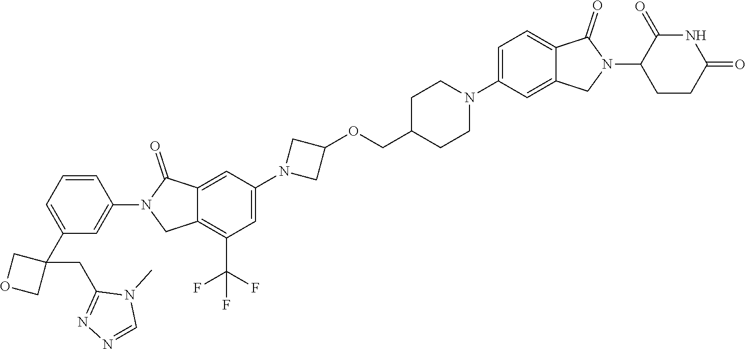 [SMILES: Cn1cnnc1CC1(c2cccc(N3Cc4c(cc(N5CC(OCC6CCN(c7ccc8c(c7)CN(C7CCC(=O)NC7=O)C8=O)CC6)C5)cc4C(F)(F)F)C3=O)c2)COC1]

To a solution of 3-aminopiperidine-2,6-dione hydrochloride (15 mg, 0.09 mmol) and methyl 2-formyl-4-[4-[[1-[2-[3-[3-[(4-methyl-1,2,4-triazol-3-yl)methyl]oxetan-3-yl]phenyl]-3-oxo-7-(trifluoromethyl)isoindolin-5-yl]azetidin-3-yl]oxymethyl]-1-piperidyl]benzoate (73 mg, 0.10 mmol) in methanol (2 mL) was added sodium acetate (16 mg, 0.2 mmol), acetic acid (17 mg, 0.3 mmol) and then sodium cyanoborohydride (18 mg, 0.3 mmol). The mixture was stirred at 40° C. for 11 h, then filtered and concentrated. The residue was purified by preparative HPLC (column: Unisil 3-100 C18 Ultra 150×50 mm×3 um; mobile phase: [water(FA)-ACN]; B %: 41%-71%, 7 min) to afford 3-[5-[4-[[1-[2-[3-[3-[(4-methyl-1,2,4-triazol-3-yl)methyl]oxetan-3-yl]phenyl]-3-oxo-7-(trifluoromethyl)isoindolin-5-yl]azetidin-3-yl]oxymethyl]-1-piperidyl]-1-oxo-isoindolin-2-yl]piperidine-2,6-dione (12 mg, 15%) as a white solid. MS (ESI) m/z: 839.2 [M+H]⁺; ¹H NMR (400 MHz, DMSO-d₆) δ 10.94 (s, 1H), 8.19 (s, 1H), 7.93-7.86 (m, 1H), 7.51 (d, J=8.8 Hz, 1H), 7.40-7.31 (m, 2H), 7.10-7.03 (m, 2H), 6.97 (d, J=4.4 Hz, 2H), 6.76 (d, J=7.6 Hz, 1H), 5.05 (dd, J=5.2, 13.2 Hz, 1H), 5.01-4.93 (m, 4H), 4.89 (d, J=6.0 Hz, 2H), 4.50-4.41 (m, 1H), 4.36-4.29 (m, 1H), 4.26-4.15 (m, 3H), 3.90 (d, J=12.4 Hz, 2H), 3.77 (dd, J=4.0, 8.4 Hz, 2H), 3.51 (s, 2H), 2.91 (s, 3H), 2.88-2.81 (m, 2H), 2.66-2.60 (m, 1H), 2.56 (s, 2H), 2.43-2.34 (m, 1H), 2.00-1.93 (m, 1H), 1.78 (d, J=12.0 Hz, 3H), 1.39-1.14 (m, 3H).

### Example 22: Exemplary synthesis of 2-(2,6-dioxo-3-piperidyl)-5-[4-[4-[[4-[[2-[3-[3-[(4-methyl-1,2,4-triazol-3-yl)methyl]oxetan-3-yl]phenyl]-3-oxo-7-(trifluoromethyl)isoindolin-5-yl]methyl]piperazin-1-yl]methyl]cyclohexoxy]-1-piperidyl]isoindoline-1,3-dione

### Step 1: Preparation of 5-[4-[4-(dimethoxymethyl)cyclohexoxy]-1-piperidyl]-2-(2,6-dioxo-3-piperidyl)isoindoline-1,3-dione

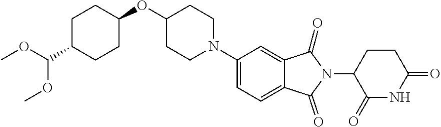 [SMILES: COC(OC)[C@H]1CC[C@H](OC2CCN(c3ccc4c(c3)C(=O)N(C3CCC(=O)NC3=O)C4=O)CC2)CC1]

To a solution of 4-[4-(dimethoxymethyl)cyclohexoxy]piperidine (200 mg, 0.8 mmol) and 2-(2,6-dioxo-3-piperidyl)-5-fluoro-isoindoline-1,3-dione (215 mg, 0.8 mmol) in dimethylsulfoxide (3 mL) was added diisopropylethylamine (301 mg, 2.3 mmol). The mixture was stirred at 100° C. for 5 h, then partitioned between water (30 mL) and ethyl acetate (10 mL). The organic phase was separated, washed with brine (20 mL), dried over anhydrous sodium sulfate, filtered, and concentrated. The residue was purified by column chromatography (dichloromethane:methanol=1/0 to 30/1) to give 5-[4-[4-(dimethoxymethyl)cyclohexoxy]-1-piperidyl]-2-(2,6-dioxo-3-piperidyl)isoindoline-1,3-dione (300 mg, 75%) as a yellow solid. MS (ESI) m/z: 514.3 [M+H]⁺; ¹H NMR (400 MHz, CDCl₃) δ 8.03 (s, 1H), 7.69 (d, J=8.4 Hz, 1H), 7.30 (d, J=2.0 Hz, 1H), 7.07 (dd, J=2.4, 8.8 Hz, 1H), 4.96 (dd, J=5.2, 12.4 Hz, 1H), 4.02 (d, J=6.8 Hz, 1H), 3.82-3.69 (m, 3H), 3.37 (s, 6H), 3.35-3.22 (m, 3H), 2.95-2.88 (m, 1H), 2.88-2.81 (m, 1H), 2.80-2.73 (m, 1H), 2.22-2.11 (m, 1H), 2.08-2.04 (m, 2H), 1.99-1.91 (m, 2H), 1.88 (d, J=12.8 Hz, 2H), 1.71 (dd, J=4.4, 8.4 Hz, 2H), 1.33-1.27 (m, 2H), 1.27-1.24 (m, 1H), 1.15-1.00 (m, 2H).

### Step 2: Preparation of 4-[[1-[2-(2,6-dioxo-3-piperidyl)-1,3-dioxo-isoindolin-5-yl]-4-piperidyl]oxy]cyclohexanecarbaldehyde

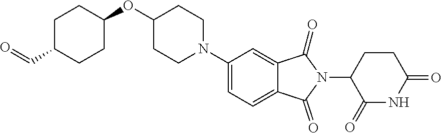 [SMILES: O=C[C@H]1CC[C@H](OC2CCN(c3ccc4c(c3)C(=O)N(C3CCC(=O)NC3=O)C4=O)CC2)CC1]

To a solution of 5-[4-[4-(dimethoxymethyl)cyclohexoxy]-1-piperidyl]-2-(2,6-dioxo-3-piperidyl)isoindoline-1,3-dione (80 mg, 0.2 mmol) in dichloromethane (3 mL) was added trifluoroacetic acid (54 mg, 0.5 mmol). The mixture was stirred at 25° C. for 1 h, then concentrated under reduced pressure to give 4-[[1-[2-(2,6-dioxo-3-piperidyl)-1,3-dioxo-isoindolin-5-yl]-4-piperidyl]oxy]cyclohexanecarbaldehyde trifluoroacetate (90 mg, crude) as a yellow oil. MS (ESI) m/z: 468.3 [M+H]⁺.

### Step 3: Preparation of tert-butyl 4-[[2-[3-[3-[(4-methyl-1,2,4-triazol-3-yl)methyl]oxetan-3-yl]phenyl]-3-oxo-7-(trifluoromethyl)isoindolin-5-yl]methyl]piperazine-1-carboxylate

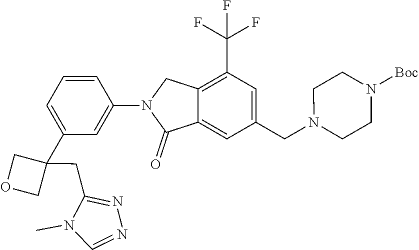 [SMILES: Cn1cnnc1CC1(c2cccc(N3Cc4c(cc(CN5CCN(C(=O)OC(C)(C)C)CC5)cc4C(F)(F)F)C3=O)c2)COC1]

To a solution of 2-[3-[3-[(4-methyl-1,2,4-triazol-3-yl)methyl]oxetan-3-yl]phenyl]-3-oxo-7-(trifluoromethyl)isoindoline-5-carbaldehyde (150 mg, 0.3 mmol) in dichloromethane (3 mL) was added tert-butyl piperazine-1-carboxylate (92 mg, 0.5 mmol). The mixture was stirred at 25° C. for 10 h. Then sodium triacetoxyborohydride (209 mg, 1 mmol) was added and the mixture was stirred at 25° C. for 1 h. The reaction mixture was concentrated and the residue was purified by preparative TLC (dichloromethane:methanol=10:1) to give tert-butyl 4-[[2-[3-[3-[(4-methyl-1,2,4-triazol-3-yl)methyl]oxetan-3-yl]phenyl]-3-oxo-7-(trifluoromethyl)isoindolin-5-yl]methyl]piperazine-1-carboxylate (160 mg, 78%) as a colorless oil. MS (ESI) m/z: 627.3 [M+H]⁺.

### Step 4: Preparation of 2-[3-[3-[(4-methyl-1,2,4-triazol-3-yl)methyl]oxetan-3-yl]phenyl]-6-(piperazin-1-ylmethyl)-4-(trifluoromethyl)isoindolin-1-one

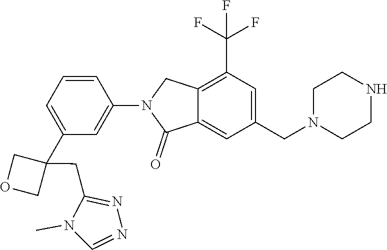 [SMILES: Cn1cnnc1CC1(c2cccc(N3Cc4c(cc(CN5CCNCC5)cc4C(F)(F)F)C3=O)c2)COC1]

To a solution of tert-butyl 4-[[2-[3-[3-[(4-methyl-1,2,4-triazol-3-yl)methyl]oxetan-3-yl]phenyl]-3-oxo-7-(trifluoromethyl)isoindolin-5-yl]methyl]piperazine-1-carboxylate (150 mg, 0.2 mmol) in dichloromethane (3 mL) was added trifluoroacetic acid (82 mg, 0.7 mmol). The mixture was stirred at 25° C. for 1 h, then concentrated under reduced pressure to give 2-[3-[3-[(4-methyl-1,2,4-triazol-3-yl)methyl]oxetan-3-yl]phenyl]-6-(piperazin-1-ylmethyl)-4-(trifluoromethyl)isoindolin-1-one trifluoroacetate (151 mg, 98%) as a colorless oil. MS (ESI) m/z: 527.3 [M+H]⁺.

### Step 5: Preparation of 2-(2,6-dioxo-3-piperidyl)-5-[4-[4-[[4-[[2-[3-[3-[(4-methyl-1,2,4-triazol-3-yl)methyl]oxetan-3-yl]phenyl]-3-oxo-7-(trifluoromethyl)isoindolin-5-yl]methyl]piperazin-1-yl]methyl]cyclohexoxy]-1-piperidyl]isoindoline-1,3-dione

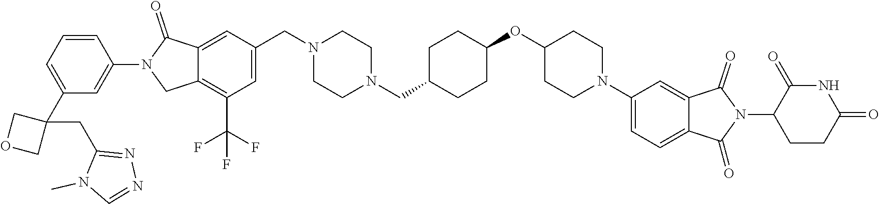 [SMILES: Cn1cnnc1CC1(c2cccc(N3Cc4c(cc(CN5CCN(C[C@H]6CC[C@H](OC7CCN(c8ccc9c(c8)C(=O)N(C8CCC(=O)NC8=O)C9=O)CC7)CC6)CC5)cc4C(F)(F)F)C3=O)c2)COC1]

To a solution of 2-[3-[3-[(4-methyl-1,2,4-triazol-3-yl)methyl]oxetan-3-yl]phenyl]-6-(piperazin-1-ylmethyl)-4-(trifluoromethyl)isoindolin-1-one trifluoroacetate (75 mg, 0.1 mmol) and 4-[[1-[2-(2,6-dioxo-3-piperidyl)-1,3-dioxo-isoindolin-5-yl]-4-piperidyl]oxy]cyclohexanecarbaldehyde trifluoroacetate (75 mg, 0.1 mmol) in dichloromethane (3 mL) was added triethylamine (36 mg, 0.4 mmol). The mixture was stirred at 25° C. for 10 h. Then sodium triacetoxyborohydride (75 mg, 0.4 mmol) was added and stirred at 25° C. for 1 h. The reaction mixture was concentrated. The residue was purified by preparative HPLC (column: Phenomenex Luna C18 150×25 mm×10 um; mobile phase: [water(FA)-ACN]; B %: 18%-38%, 10 min) to give 2-(2,6-dioxo-3-piperidyl)-5-[4-[4-[[4-[[2-[3-[3-[(4-methyl-1,2,4-triazol-3-yl)methyl]oxetan-3-yl]phenyl]-3-oxo-7-(trifluoromethyl)isoindolin-5-yl]methyl]piperazin-1-yl]methyl]cyclohexoxy]-1-piperidyl]isoindoline-1,3-dione (14.1 mg, 12%) as a yellow solid. MS (ESI) m/z: 478.6 [M+H]⁺; ¹H NMR (400 MHz, DMSO-d₆) δ 11.08 (s, 1H), 8.19 (s, 1H), 7.98 (s, 1H), 7.92 (s, 1H), 7.89 (d, J=9.6 Hz, 1H), 7.65 (d, J=8.4 Hz, 1H), 7.40 (s, 1H), 7.38-7.34 (m, 1H), 7.34-7.31 (m, 1H), 7.24 (dd, J=2.0, 9.2 Hz, 1H), 6.77 (d, J=8.0 Hz, 1H), 5.10 (s, 2H), 5.08-5.03 (m, 1H), 4.97 (d, J=6.0 Hz, 2H), 4.89 (d, J=6.0 Hz, 2H), 3.82-3.75 (m, 2H), 3.73-3.66 (m, 3H), 3.52 (s, 2H), 3.28-3.18 (m, 3H), 2.91 (s, 4H), 2.59 (d, J=16.0 Hz, 1H), 2.50-2.46 (m, 3H), 2.33 (d, J=1.6 Hz, 6H), 2.07 (d, J=8.0 Hz, 2H), 2.04-1.99 (m, 1H), 1.92 (d, J=10.0 Hz, 2H), 1.86 (dd, J=4.0, 9.2 Hz, 2H), 1.79-1.72 (m, 2H), 1.50-1.38 (m, 3H), 1.17-1.07 (m, 2H), 0.92-0.81 (m, 2H).

### Example 74: Exemplary synthesis of 3-{4-fluoro-5-[4-({1-[(1-{[(3R)-1-{[2-(3-{3-[(4-methyl-4H-1,2,4-triazol-3-yl)methyl]oxetan-3-yl}phenyl)-3-oxo-7-(trifluoromethyl)-2,3-dihydro-1H-isoindol-5-yl]methyl}piperidin-3-yl]methyl}piperidin-4-yl)methyl]piperidin-4-yl}methyl)piperazin-1-yl]-1-oxo-2,3-dihydro-1H-isoindol-2-yl}piperidine-2,6-dione

### Step 1: Preparation of methyl 3,4-difluoro-2-methyl-benzoate

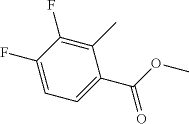

To a solution of 3,4-difluoro-2-methyl-benzoic acid (50.0 g, 290 mmol) in methanol (500 mL) was dropwise added thionyl chloride (63.2 mL, 871 mmol). The mixture was stirred at 80° C. for 12 h, then slowly poured into ice water (1 L) at 0° C., and mixture was filtered. The solid was suspended in ethyl acetate (10 mL), filtered to give methyl 3,4-difluoro-2-methyl-benzoate (51 g, 94%) as a white solid. ¹H NMR (400 MHz, CDCl₃) δ 7.74-7.68 (m, 1H), 7.08-6.99 (m, 1H), 3.90 (s, 3H), 2.60-2.53 (m, 3H).

### Step 2: Preparation of methyl 2-(bromomethyl)-3,4-difluoro-benzoate

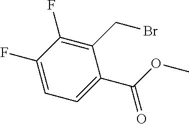

To a mixture of methyl 3,4-difluoro-2-methyl-benzoate (40.0 g, 215 mmol) in 1,2-dichloroethane (400 mL) was added n-bromosuccinimide (57.4 g, 322 mmol) and benzoyl peroxide (520 mg, 2.1 mmol). The mixture was degassed and purged with nitrogen for 3 times, then the mixture was stirred at 80° C. for 2 h. The mixture was cooled to 20° C., filtered and concentrated under reduce pressure. The residue was purified by silica gel column chromatography (petroleum ether:ethyl acetate=1:0 to 20:1) to give methyl 2-(bromomethyl)-3,4-difluoro-benzoate (51.0 g, 89%) as a colorless oil. ¹H NMR (400 MHz, CDCl₃) δ 7.85-7.80 (m, 1H), 7.16-7.23 (m, 1H), 5.02 (d, J=2.2 Hz, 2H), 3.96 (s, 3H).

### Step 3: Preparation of 3-(4,5-difluoro-1-oxo-isoindolin-2-yl)piperidine-2,6-dione

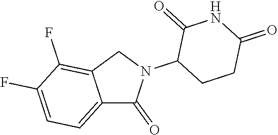 [SMILES: O=C1CCC(N2Cc3c(ccc(F)c3F)C2=O)C(=O)N1]

To a mixture of methyl 2-(bromomethyl)-3,4-difluoro-benzoate (51.0 g, 192 mmol) and 3-aminopiperidine-2,6-dione hydrochloride (33.2 g, 202 mmol) in N,N-dimethylformamide (600 mL) was added diisopropylethylamine (100 mL, 577 mmol). The mixture was stirred at 40° C. for 1 h, then 110° C. for 12 h. The mixture was poured into water (800 mL), filtered and the solid was suspended in ethyl acetate (500 mL), filtered and the solid was dried to afford 3-(4,5-difluoro-1-oxo-isoindolin-2-yl)piperidine-2,6-dione (29.70 g, 55%) as a gray solid. ¹H NMR (400 MHz, DMSO-d₆) δ 11.02 (s, 1H), 7.66-7.58 (m, 2H), 5.11 (dd, J=5.2, 13.2 Hz, 1H), 4.66-4.59 (m, 1H), 4.49-4.42 (m, 1H), 2.97-2.86 (m, 1H), 2.65-2.57 (m, 1H), 2.46-2.37 (m, 1H), 2.05-1.98 (m, 1H).

### Step 4: Preparation of [(3S)-3-piperidyl]methanol

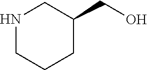

To a solution of tert-butyl (3S)-3-(hydroxymethyl)piperidine-1-carboxylate (10 g, 46 mmol) in dichloromethane (100 mL) was added trifluoroacetic acid (50.0 mL, 675 mmol). The mixture was stirred at 25° C. for 0.5 h, then concentrated to afford [(3S)-3-piperidyl]methanol trifluoroacetate (10 g, 94%) as a yellow oil.

### Step 5: Preparation of benzyl (3S)-3-(hydroxymethyl)piperidine-1-carboxylate

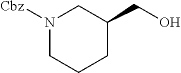 [SMILES: O=C(OCc1ccccc1)N1CCC[C@H](CO)C1]

To a solution of [(3S)-3-piperidyl]methanol trifluoroacetate (10 g, 44 mmol) in dichloromethane (100 mL) was added triethylamine (18.0 mL, 131 mmol,) and benzyl carbonochloridate (9.0 mL, 66 mmol). The mixture was stirred at 25° C. for 10 h, then concentrated under reduced pressure. The residue was purified by column chromatography on silica gel (petroleum ether/ethyl acetate=15/1 to 5/1) to give benzyl (3S)-3-(hydroxymethyl)piperidine-1-carboxylate (9.4 g, 86%) as an off white oil. MS (ESI) m/z: 250.2 [M+1]⁺; ¹H NMR (400 MHz, CDCl₃) δ 1.75-1.86 (m, 4H) 1.87-1.95 (m, 1H) 2.92-3.07 (m, 2H) 3.47-3.64 (m, 4H) 5.15 (br d, J=3.6 Hz, 2H) 7.30-7.48 (m, 5H).

### Step 6: Preparation of benzyl (3S)-3-(p-tolylsulfonyloxymethyl)piperidine-1-carboxylate

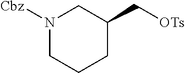 [SMILES: Cc1ccc(S(=O)(=O)OC[C@H]2CCCN(C(=O)OCc3ccccc3)C2)cc1]

To a solution of benzyl (3S)-3-(hydroxymethyl)piperidine-1-carboxylate (9.40 g, 38 mmol) in dichloromethane (100 mL) was added triethylamine (16.0 mL, 113 mmol) and p-toluenesulfonyl chloride (10.78 g, 57 mmol). The mixture was stirred at 25° C. for 10 h, then concentrated under reduced pressure. The residue was purified by column chromatography on silica gel (petroleum ether/ethyl acetate=15/1 to 1/2) to give benzyl (3S)-3-(p-tolylsulfonyloxymethyl)piperidine-1-carboxylate (14.00 g, 92%) as a yellow oil. MS (ESI) m/z: 404.2 [M+1]⁺; ¹H NMR (400 MHz, CDCl₃) δ 1.45-1.58 (m, 2H) 1.76-1.84 (m, 2H) 1.87-1.92 (m, 1H) 2.46 (s, 3H) 3.88-4.00 (m, 6H) 5.13 (s, 2H) 7.34-7.38 (m, 5H) 7.77-7.83 (m, 4H).

### Step 7: Preparation of benzyl (3R)-3-[[4-(dimethoxymethyl)-1-piperidyl]methyl]piperidine-1-carboxylate

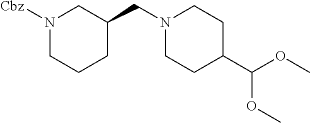 [SMILES: COC(OC)C1CCN(C[C@H]2CCCN(C(=O)OCc3ccccc3)C2)CC1]

To a solution of benzyl (3S)-3-(p-tolylsulfonyloxymethyl)piperidine-1-carboxylate (5.00 g, 12.4 mmol) and 4-(dimethoxymethyl)piperidine (3.95 g, 25 mmol) in acetonitrile (50 mL) was added potassium carbonate (5.14 g, 37 mmol). The mixture was stirred at 80° C. for 10 h, then concentrated under reduced pressure. The residue was purified by prep-HPLC (column: Phenomenex Luna C18 150×40 mm×15 um; mobile phase: [water(FA)-ACN]; B %: 13%-43%, 10 min) and prep-HPLC (column: Waters Xbridge 150×25 mm×5 um; mobile phase: [water (ammonia hydroxide v/v)-ACN]; B %: 54%-84%, 9 min) to give benzyl (3R)-3-[[4-(dimethoxymethyl)-1-piperidyl]methyl]piperidine-1-carboxylate (1.60 g, 33%) as an off white oil. MS (ESI) m/z: 391.4 [M+Na]⁺; ¹H NMR (400 MHz, CDCl₃) δ 0.98-1.20 (m, 1H) 1.32 (br dd, J=9.6, 1.6 Hz, 2H) 1.43-1.63 (m, 3H) 1.69 (br d, J=12.0 Hz, 3H) 1.77-1.95 (m, 3H) 2.05-2.23 (m, 2H) 2.37-2.68 (m, 1H) 2.71-2.99 (m, 3H) 3.36 (d, J=2.4 Hz, 6H) 4.03 (br d, J=7.6 Hz, 2H) 4.08-4.29 (m, 1H) 5.03-5.23 (m, 2H) 7.30-7.42 (m, 5H).

### Step 8: Preparation of 4-(dimethoxymethyl)-1-[[(3S)-3-piperidyl]methyl]piperidine

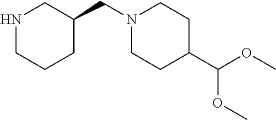 [SMILES: COC(OC)C1CCN(C[C@H]2CCCNC2)CC1]

To a solution of benzyl (3R)-3-[[4-(dimethoxymethyl)-1-piperidyl]methyl]piperidine-1-carboxylate (500 mg, 1.3 mmol) in methanol (50 mL) was added 5% palladium on carbon (200 mg). The mixture was degassed and purged with hydrogen, then stirred at 25° C. for 10 h under hydrogen (15 psi) atmosphere. The mixture was filtered and concentrated to afford 4-(dimethoxymethyl)-1-[[(3S)-3-piperidyl]methyl]piperidine (290 mg, 88%) as an off white oil.

### Step 9: Preparation of 6-[[(3R)-3-[[4-(dimethoxymethyl)-1-piperidyl]methyl]-1-piperidyl]methyl]-2-[3-[3-[(4-methyl-1,2,4-triazol-3-yl)methyl]oxetan-3-yl]phenyl]-4-(trifluoromethyl)isoindolin-1-one

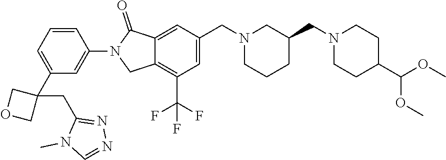 [SMILES: COC(OC)C1CCN(C[C@H]2CCCN(Cc3cc4c(c(C(F)(F)F)c3)CN(c3cccc(C5(Cc6nncn6C)COC5)c3)C4=O)C2)CC1]

To a solution of 2-[3-[3-[(4-methyl-1,2,4-triazol-3-yl)methyl]oxetan-3-yl]phenyl]-3-oxo-7-(trifluoromethyl)isoindoline-5-carbaldehyde (300 mg, 0.7 mmol), 4-(dimethoxymethyl)-1-[[(3S)-3-piperidyl]methyl]piperidine (253 mg, 0.99 mmol), triethylamine (67 mg, 0.66 mmol) and tetraisopropoxytitanium (186.8 mg, 0.66 mmol) in methanol (5 mL) was added sodium cyanoborohydride (123.9 mg, 1.97 mmol). The mixture was stirred at 40° C. for 2 h, then filtered and concentrated. The residue was purified by prep-HPLC (column: Phenomenex Luna C18 150×25 mm×10 um; mobile phase: [water(FA)-ACN]; B %: 1%-29%, 10 min) to give 6-[[(3R)-3-[[4-(dimethoxymethyl)-1-piperidyl]methyl]-1-piperidyl]methyl]-2-[3-[3-[(4-methyl-1,2,4-triazol-3-yl)methyl]oxetan-3-yl]phenyl]-4-(trifluoromethyl)isoindolin-1-one (100 mg, 22%) as a white solid. MS (ESI) m/z: 697.4 [M+Na]⁺.

### Step 10: Preparation of 1-[[(3R)-1-[[2-[3-[3-[(4-methyl-1,2,4-triazol-3-yl)methyl]oxetan-3-yl]phenyl]-3-oxo-7-(trifluoromethyl)isoindolin-5-yl]methyl]-3-piperidyl]methyl]piperidine-4-carbaldehyde

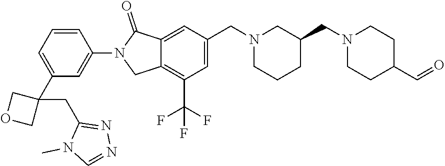 [SMILES: Cn1cnnc1CC1(c2cccc(N3Cc4c(cc(CN5CCC[C@H](CN6CCC(C=O)CC6)C5)cc4C(F)(F)F)C3=O)c2)COC1]

To a solution of 6-[[(3R)-3-[[4-(dimethoxymethyl)-1-piperidyl]methyl]-1-piperidyl]methyl]-2-[3-[3-[(4-methyl-1,2,4-triazol-3-yl)methyl]oxetan-3-yl]phenyl]-4-(trifluoromethyl)isoindolin-1-one (100 mg, 0.14 mmol) in dichloromethane (2 mL) was added trifluoroacetic acid (1.0 mL, 13 mmol). The mixture was stirred at 25° C. for 0.5 h, then concentrated to afford 1-[[(3R)-1-[[2-[3-[3-[(4-methyl-1,2,4-triazol-3-yl)methyl]oxetan-3-yl]phenyl]-3-oxo-7-(trifluoromethyl)isoindolin-5-yl]methyl]-3-piperidyl]methyl]piperidine-4-carbaldehyde (93 mg, 99%) as an off white oil. MS (ESI) m/z: 651.4[M+1]⁺.

### Step 11: Preparation of tert-butyl 4-[[4-[2-(2,6-dioxo-3-piperidyl)-4-fluoro-1-oxo-isoindolin-5-yl]piperazin-1-yl]methyl]piperidine-1-carboxylate

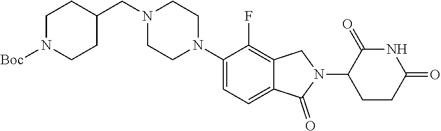 [SMILES: CC(C)(C)OC(=O)N1CCC(CN2CCN(c3ccc4c(c3F)CN(C3CCC(=O)NC3=O)C4=O)CC2)CC1]

To a solution of tert-butyl 4-(piperazin-1-ylmethyl)piperidine-1-carboxylate (850 mg, 3 mmol) and 3-(4,5-difluoro-1-oxo-isoindolin-2-yl)piperidine-2,6-dione (600 mg) in dimethylsulfoxide (2 mL) was added diisopropylethylamine (1.9 mL, 11 mmol). The mixture was stirred at 130° C. for 10 h, then filtered and concentrated. The residue was purified by prep-HPLC (column: Phenomenex luna C18 150×40 mm×15 um; mobile phase: [water(FA)-ACN]; B %: 12%-42%, 10 min) to give tert-butyl 4-[[4-[2-(2,6-dioxo-3-piperidyl)-4-fluoro-1-oxo-isoindolin-5-yl]piperazin-1-yl]methyl]piperidine-1-carboxylate (340 mg, 29%) as a yellow solid. MS (ESI) m/z: 544.3 [M+1]⁺.

### Step 12: Preparation of 3-[4-fluoro-1-oxo-5-[4-(4-piperidylmethyl)piperazin-1-yl]isoindolin-2-yl]piperidine-2,6-dione

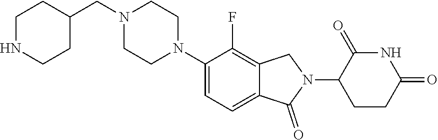 [SMILES: O=C1CCC(N2Cc3c(ccc(N4CCN(CC5CCNCC5)CC4)c3F)C2=O)C(=O)N1]

To a solution of tert-butyl 4-[[4-[2-(2,6-dioxo-3-piperidyl)-4-fluoro-1-oxo-isoindolin-5-yl]piperazin-1-yl]methyl]piperidine-1-carboxylate (80 mg, 0.2 mmol) in dichloromethane (2 mL) was added trifluoroacetic acid (1.23 g, 10.8 mmol). The mixture was stirred at 25° C. for 0.5 h, then concentrated under reduced pressure to give 3-[4-fluoro-1-oxo-5-[4-(4-piperidylmethyl)piperazin-1-yl]isoindolin-2-yl]piperidine-2,6-dione (65 mg, 99%) as a brown oil, which was used in the next step directly. MS (ESI) m/z: 444.2 [M+1]⁺.

### Step 13: Preparation of 3-[4-fluoro-5-[4-[[1-[[1-[[(3R)-1-[[2-[3-[3-[(4-methyl-1,2,4-triazol-3-yl)methyl]oxetan-3-yl]phenyl]-3-oxo-7-(trifluoromethyl)isoindolin-5-yl]methyl]-3-piperidyl]methyl]-4-piperidyl]methyl]-4-piperidyl]methyl]piperazin-1-yl]-1-oxo-isoindolin-2-yl]piperidine-2,6-dione

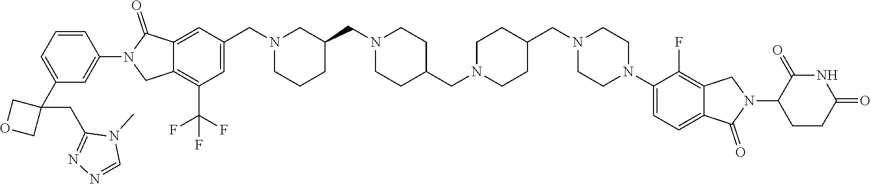 [SMILES: Cn1cnnc1CC1(c2cccc(N3Cc4c(cc(CN5CCC[C@H](CN6CCC(CN7CCC(CN8CCN(c9ccc%10c(c9F)CN(C9CCC(=O)NC9=O)C%10=O)CC8)CC7)CC6)C5)cc4C(F)(F)F)C3=O)c2)COC1]

To a solution of 1-[[(3R)-1-[[2-[3-[3-[(4-methyl-1,2,4-triazol-3-yl)methyl]oxetan-3-yl]phenyl]-3-oxo-7-(trifluoromethyl)isoindolin-5-yl]methyl]-3-piperidyl]methyl]piperidine-4-carbaldehyde (38 mg, 0.06 mmol), 3-[4-fluoro-1-oxo-5-[4-(4-piperidylmethyl)piperazin-1-yl]isoindolin-2-yl]piperidine-2,6-dione (39 mg, 0.09 mmol), triethylamine (6 mg, 0.06 mmol) and tetraisopropoxytitanium (17 mg, 0.06 mmol) in dichloromethane (1 mL) and dimethylformamide (1 mL) was added sodium cyanoborohydride (7 mg, 0.1 mmol). The mixture was stirred at 25° C. for 4 h, then filtered and concentrated. The residue was purified by prep-HPLC (column: Phenomenex Luna C18 150×25 mm×10 um; mobile phase: [water(FA)-ACN]; B %: 1%-26%, 10 min) to give 3-[4-fluoro-5-[4-[[1-[[1-[[(3R)-1-[[2-[3-[3-[(4-methyl-1,2,4-triazol-3-yl)methyl]oxetan-3-yl]phenyl]-3-oxo-7-(trifluoromethyl)isoindolin-5-yl]methyl]-3-piperidyl]methyl]-4-piperidyl]methyl]-4-piperidyl]methyl]piperazin-1-yl]-1-oxo-isoindolin-2-yl]piperidine-2,6-dione (15.9 mg, 23%) as a colorless solid. MS (ESI) m/z: 1078.4 [M+1]⁺; ¹H NMR (400 MHz, DMSO-d₆) δ 10.99 (s, 1H), 8.20 (s, 1H), 8.15 (s, 3H), 7.92-8.00 (m, 2H), 7.89 (br d, J=8.4 Hz, 1H), 7.49 (d, J=8.4 Hz, 1H), 7.41 (s, 1H), 7.36 (t, J=8.0 Hz, 1H), 7.16 (t, J=8.0 Hz, 1H), 6.78 (d, J=7.6 Hz, 1H), 5.11 (s, 2H), 5.07 (br d, J=4.8 Hz, 1H), 4.97 (d, J=6.0 Hz, 2H), 4.89 (d, J=6.0 Hz, 2H), 4.49 (d, J=17.2 Hz, 1H), 4.32 (d, J=16.8 Hz, 1H), 3.62-3.74 (m, 2H), 3.52 (s, 2H), 3.13 (br s, 4H), 2.91 (s, 3H), 2.77-2.89 (m, 5H), 2.61 (br d, J=6.8 Hz, 2H), 2.40 (br d, J=4.8 Hz, 2H), 2.14-2.23 (m, 7H), 1.93-2.07 (m, 6H), 1.74-1.90 (m, 4H), 1.61-1.71 (m, 6H), 1.47-1.56 (m, 3H), 1.04-1.18 (m, 4H), 0.89-1.01 ppm (m, 2H).

### Example 145: Exemplary synthesis of 3-[4-fluoro-1-oxo-5-(4-{[(1r, 4r)-4-[(4-{[(3S)-1-[(2-{3-[2-(4-methyl-4H-1,2,4-triazol-3-yl)cyclopropyl]phenyl}-3-oxo-7-(trifluoromethyl)-2,3-dihydro-1H-isoindol-5-yl)methyl]piperidin-3-yl]methyl}piperazin-1-yl)methyl]cyclohexyl]oxy}piperidin-1-yl)-2,3-dihydro-1H-isoindol-2-yl]piperidine-2,6-dione

### Step 1: Preparation of ethyl (Z)-3-(3-bromophenyl)prop-2-enoate

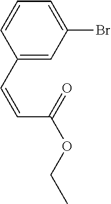

To a solution of 3-bromobenzaldehyde (5.00 g, 27 mmol) in dichloromethane (100 mL) was added ethyl 2-(triphenyl-λ5-phosphanylidene)acetate (10.36 g, 30 mmol). The mixture was stirred at 25° C. for 10 h, then concentrated under reduced pressure. The residue was purified by column chromatography on silica gel (petroleum ether/ethyl acetate=1/0 to 20/1) to give ethyl (Z)-3-(3-bromophenyl)prop-2-enoate (6.50 g, 94%) as an off white oil. ¹H NMR (400 MHz, CDCl₃) δ 7.59 (s, 1H), 7.52 (d, J=16.0 Hz, 1H), 7.43 (br d, J=7.6 Hz, 1H), 7.36 (d, J=7.6 Hz, 1H), 7.14-7.23 (m, 1H), 6.35 (d, J=16.0 Hz, 1H), 4.19 (q, J=7.2 Hz, 2H), 1.26 ppm (t, J=7.2 Hz, 3H).

### Step 2: Preparation of ethyl 2-(3-bromophenyl)cyclopropanecarboxylate

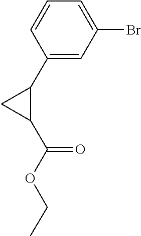

Under nitrogen, 60% sodium hydride (367 mg, 9.2 mmol) was initially charged in tetrahydrofuran (25 mL) and trimethylsulfoxonium iodide (3.36 g, 15.3 mmol) was added in one portion at 25° C. After the evolution of gas had ceased, ethyl (Z)-3-(3-bromophenyl)prop-2-enoate (3.00 g, 11.8 mmol) in tetrahydrofuran (10 mL) was slowly added. Then the mixture was stirred at 50° C. for 10 h. The reaction was quenched by ammonium chloride and extracted with ethyl acetate 200 mL (2×100 mL). The organic layer was concentrated under reduced pressure. The residue was purified by column chromatography on silica gel (petroleum ether/ethyl acetate=1/0 to 20/1) to give ethyl 2-(3-bromophenyl)cyclopropanecarboxylate (1.30 g, 41%) as a white solid. ¹H NMR (400 MHz, CDCl₃) δ 7.23-7.29 (m, 1H), 7.16 (t, J=1.6 Hz, 1H), 7.07 (t, J=7.6 Hz, 1H), 6.96 (d, J=7.6 Hz, 1H), 4.10 (q, J=7.2 Hz, 2H), 2.41 (ddd, J=9.2, 6.4, 4.0 Hz, 1H), 1.82 (ddd, J=8.4, 5.2, 4.0 Hz, 1H), 1.51-1.58 (m, 1H), 1.23-1.26 (m, 1H), 1.15-1.22 ppm (m, 3H).

### Step 3: Preparation of 2-(3-bromophenyl)cyclopropanecarboxylic acid

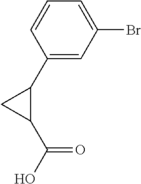 [SMILES: O=C(O)C1CC1c1cccc(Br)c1]

To a solution of ethyl 2-(3-bromophenyl)cyclopropanecarboxylate (1.30 g, 4.8 mmol) in methanol (13 mL) and water (4 mL) was added sodium hydroxide (580 mg, 14.5 mmol). The mixture was stirred at 25° C. for 2 h, then concentrated. Adjusted the pH to 4 by 1 M hydrochloric acid solution. Then the mixture was extracted by ethyl acetate 200 mL (2×100 mL). The organic layer was concentrated under reduced pressure to give 2-(3-bromophenyl)cyclopropanecarboxylic acid (1.1 g, 94%) as an off white oil. ¹H NMR (400 MHz, CDCl₃) δ 7.34-7.45 (m, 2H), 7.15-7.28 (m, 2H), 2.41 (ddd, J=9.2, 6.4, 4.0 Hz, 1H), 1.86 (ddd, J=8.4, 5.2, 4.4 Hz, 1H), 1.33-1.49 ppm (m, 2H).

### Step 4: Preparation of 1-[[2-(3-bromophenyl)cyclopropanecarbonyl]amino]-3-methyl-thiourea

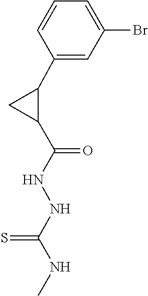 [SMILES: CNC(=S)NNC(=O)C1CC1c1cccc(Br)c1]

To a solution of 2-(3-bromophenyl)cyclopropanecarboxylic acid (1.10 g, 4.6 mmol) in N,N-dimethylformamide (10 mL) was added diisopropylethylamine (2.38 mL, 13.7 mmol), o-(7-azabenzotriazol-1-yl)-n,n,n′,n′-tetramethyluronium hexafluorophosphate (2.08 g, 5.5 mmol) and 1-amino-3-methyl-thiourea (720 mg, 6.8 mmol). The mixture was stirred at 25° C. for 3 h. Diluted with water (50 mL) and extracted with ethyl acetate (3×20 mL). The combined organic phase was washed with brine (300 mL), dried over anhydrous sodium sulfate, filtered, and concentrated under reduced pressure to give the crude product, which was used in the next step without further purification. MS (ESI) m/z: 329.2 [M+1]⁺.

### Step 5: Preparation of 5-[2-(3-bromophenyl)cyclopropyl]-4-methyl-1,2,4-triazole-3-thiol

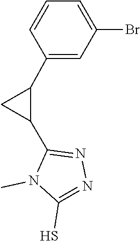 [SMILES: Cn1c(S)nnc1C1CC1c1cccc(Br)c1]

To a solution of 1-[[2-(3-bromophenyl)cyclopropanecarbonyl]amino]-3-methyl-thiourea (1.50 g, 4.6 mmol) in tetrahydrofuran (60 mL) was added sodium hydroxide (1 M, 6.9 mL). The mixture was stirred at 50° C. for 5 h. The mixture was dropwise added into saturated ammonium chloride solution (200 mL) and extracted with ethyl acetate 200 mL (2×100 mL). The organic layer was concentrated. The residue was purified by column chromatography on silica gel (petroleum ether/ethyl acetate=10/1 to 1/1) to give 5-[2-(3-bromophenyl)cyclopropyl]-4-methyl-1,2,4-triazole-3-thiol (1.40 g, 99%) as a yellow oil. MS (ESI) m/z: 312.1 [M+1]⁺.

### Step 6: Preparation of 3-[2-(3-bromophenyl)cyclopropyl]-4-methyl-1,2,4-triazole

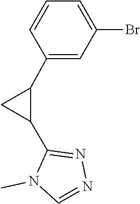 [SMILES: Cn1cnnc1C1CC1c1cccc(Br)c1]

To a solution of 5-[2-(3-bromophenyl)cyclopropyl]-4-methyl-1,2,4-triazole-3-thiol (1.40 g, 4.5 mmol) in dichloromethane (15 mL) was added acetic acid (3.61 mL, 63.2 mmol) and 30% hydrogen peroxide (1.21 mL, 12.6 mmol) at 0° C. The mixture was stirred at 25° C. for 1 h. Dichloromethane (10 mL) and water (20 mL) were added before the mixture was extracted with dichloromethane (3×20 mL). The combined organic layers were washed with saturated sodium thiosulphate solution (2×10 mL) and brine (100 mL), dried over anhydrous sodium sulfate, filtered, and concentrated. The crude product was purified by prep-HPLC (column: Phenomenex Luna C18 150×40 mm×15 um; mobile phase: [water(FA)-ACN]; B %: 22%-52%, 10 min) to give 3-[2-(3-bromophenyl)cyclopropyl]-4-methyl-1,2,4-triazole (590 mg, 59%) as a white solid. MS (ESI) m/z: 278.1 [M+1]⁺; ¹H NMR (400 MHz, DMSO-d₆) δ 8.35 (s, 1H), 7.46 (d, J=1.2 Hz, 1H), 7.36-7.44 (m, 1H), 7.23-7.31 (m, 2H), 3.63 (s, 3H), 2.37-2.48 (m, 2H), 1.61 (ddd, J=8.8, 5.6, 4.8 Hz, 1H), 1.52 ppm (ddd, J=8.8, 6.0, 4.8 Hz, 1H).

### Step 7: Preparation of 6-(dimethoxymethyl)-2-[3-[2-(4-methyl-1,2,4-triazol-3-yl)cyclopropyl]phenyl]-4-(trifluoromethyl)isoindolin-1-one

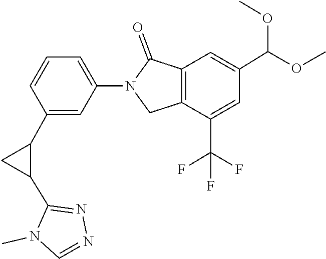 [SMILES: COC(OC)c1cc2c(c(C(F)(F)F)c1)CN(c1cccc(C3CC3c3nncn3C)c1)C2=O]

To a solution of 3-[2-(3-bromophenyl)cyclopropyl]-4-methyl-1,2,4-triazole (300 mg, 1.1 mmol) and 6-(dimethoxymethyl)-4-(trifluoromethyl)isoindolin-1-one (297 mg, 1.1 mmol) in dioxane (15 mL) was added cesium carbonate (1.05 g, 3.2 mmol) and Xphos Pd G4 (92.81 mg, 108 umol). The mixture was purged by nitrogen for 3 times and stirred at 90° C. for 10 h, THEN concentrated. The residue was purified by column chromatography (petroleum ether/ethyl acetate=3/1 to dichloromethane:methanol=10/1) to give 6-(dimethoxymethyl)-2-[3-[2-(4-methyl-1,2,4-triazol-3-yl)cyclopropyl]phenyl]-4-(trifluoromethyl)isoindolin-1-one (390 mg, 77%) as a white solid. MS (ESI) m/z: 473.2 [M+1]⁺; ¹H NMR (400 MHz, DMSO-d₆) δ 8.22 (s, 1H), 8.07 (s, 1H), 7.85-8.03 (m, 2H), 7.59 (dd, J=8.0, 1.6 Hz, 1H), 7.41 (t, J=8.0 Hz, 1H), 7.07 (d, J=7.6 Hz, 1H), 5.56 (s, 1H), 5.05 (s, 2H), 3.73 (s, 3H), 3.39 (s, 6H), 2.65-2.76 (m, 1H), 2.07-2.14 (m, 1H), 1.94 (dt, J=9.2, 5.2 Hz, 1H), 1.62-1.67 ppm (m, 1H).

### Step 8: Preparation of 2-[3-[2-(4-methyl-1,2,4-triazol-3-yl)cyclopropyl]phenyl]-3-oxo-7-(trifluoromethyl)isoindoline-5-carbaldehyde

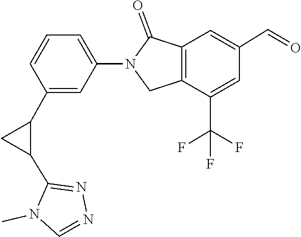 [SMILES: Cn1cnnc1C1CC1c1cccc(N2Cc3c(cc(C=O)cc3C(F)(F)F)C2=O)c1]

To a solution of 6-(dimethoxymethyl)-2-[3-[2-(4-methyl-1,2,4-triazol-3-yl)cyclopropyl]phenyl]-4-(trifluoromethyl)isoindolin-1-one (100 mg, 0.2 mmol) in dichloromethane (2 mL) was added trifluoroacetic acid (1.0 mL, 13 mmol). The mixture was stirred at 25° C. for 0.5 h, then concentrated under reduced pressure to give 2-[3-[2-(4-methyl-1,2,4-triazol-3-yl)cyclopropyl]phenyl]-3-oxo-7-(trifluoromethyl)isoindoline-5-carbaldehyde (90 mg, 99%) as a yellow oil. MS (ESI) m/z: 427.3[M+1]⁺.

### Step 9: Preparation of 3-[4-fluoro-5-[4-[4-[[4-[[(3S)-1-[[2-[3-[2-(4-methyl-1,2,4-triazol-3-yl)cyclopropyl]phenyl]-3-oxo-7-(trifluoromethyl)isoindolin-5-yl]methyl]-3-piperidyl]methyl]piperazin-1-yl]methyl]cyclohexoxy]-1-piperidyl]-1-oxo-isoindolin-2-yl]piperidine-2,6-dione

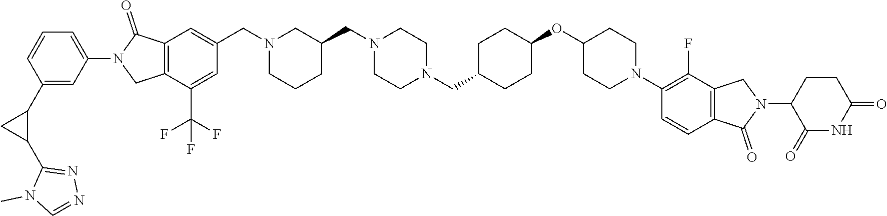 [SMILES: Cn1cnnc1C1CC1c1cccc(N2Cc3c(cc(CN4CCC[C@H](CN5CCN(C[C@H]6CC[C@H](OC7CCN(c8ccc9c(c8F)CN(C8CCC(=O)NC8=O)C9=O)CC7)CC6)CC5)C4)cc3C(F)(F)F)C2=O)c1]

To a solution of 2-[3-[2-(4-methyl-1,2,4-triazol-3-yl)cyclopropyl]phenyl]-3-oxo-7-(trifluoromethyl)isoindoline-5-carbaldehyde (85 mg, 0.2 mmol) and 3-[4-fluoro-1-oxo-5-[4-[4-[[4-[[(3S)-3-piperidyl]methyl]piperazin-1-yl]methyl]cyclohexoxy]-1-piperidyl]isoindolin-2-yl]piperidine-2,6-dione trifluoroacetate (150 mg, 0.2 mmol) in dichloromethane (2 mL) and N,N-dimethylformamide (2 mL) was added triethylamine (20 mg, 0.2 mmol) and tetraisopropoxytitanium (57 mg, 0.2 mmol). The mixture was stirred at 25° C. for 1 h. Then sodium cyanoborohydride (38 mg, 0.6 mmol) was added and stirred at 25° C. for 5 h. The mixture was filtered and concentrated. The residue was purified by prep-HPLC (column: UniSil 3-100 C18 Ultra (150×25 mm×3 um); mobile phase: [water(FA)-ACN]; B %: 12%-42%, 7 min) and prep-HPLC (column: Waters Xbridge 150×25 mm×5 um; mobile phase: [water(NH4HCO3)-ACN]; B %: 54%-84%, 8 min) to give 3-[4-fluoro-5-[4-[4-[[4-[[(3S)-1-[[2-[3-[2-(4-methyl-1,2,4-triazol-3-yl)cyclopropyl]phenyl]-3-oxo-7-(trifluoromethyl)isoindolin-5-yl]methyl]-3-piperidyl]methyl]piperazin-1-yl]methyl]cyclohexoxy]-1-piperidyl]-1-oxo-isoindolin-2-yl]piperidine-2,6-dione (10.6 mg, 5%) as a white solid. MS (ESI) m/z: 1050.1 [M+1]⁺; ¹H NMR (400 MHz, DMSO-d₆) δ 10.74-11.25 (m, 1H), 8.37 (s, 1H), 7.89-8.06 (m, 2H), 7.72-7.88 (m, 2H), 7.30-7.54 (m, 2H), 7.03-7.24 (m, 2H), 5.22 (br s, 2H), 5.08 (dd, J=13.2, 5.2 Hz, 1H), 4.41-4.57 (m, 1H), 4.31 (d, J=16.8 Hz, 1H), 3.69 (s, 1H), 3.55-3.68 (m, 5H), 2.85-2.98 (m, 3H), 2.74-2.83 (m, 1H), 2.57-2.72 (m, 4H), 2.33-2.45 (m, 4H), 2.18-2.31 (m, 6H), 2.03-2.15 (m, 4H), 1.98 (br d, J=7.2 Hz, 3H), 1.90 (br dd, J=6.8, 4.0 Hz, 4H), 1.62-1.79 (m, 7H), 1.44-1.61 (m, 5H), 1.30-1.41 (m, 1H), 1.05-1.17 (m, 2H), 0.89-0.98 (m, 1H), 0.76-0.88 ppm (m, 2H).

### Example 146: Exemplary synthesis of 3-[3-[6-[7-[1-[2-(2,6-dioxo-3-piperidyl)-1,3-dioxo-isoindolin-5-yl]piperidine-4-carbonyl]-2,7-diazaspiro[3.5]nonan-2-yl]-1-oxo-4-(trifluoromethyl)isoindolin-2-yl]phenyl]-3-[(4-methyl-1,2,4-triazol-3-yl)methyl]cyclobutanecarbonitrile

### Step 1: Preparation of 1-[2-(2,6-dioxo-3-piperidyl)-1,3-dioxo-isoindolin-5-yl]piperidine-4-carboxylic acid

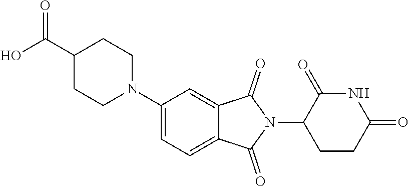 [SMILES: O=C1CCC(N2C(=O)c3ccc(N4CCC(C(=O)O)CC4)cc3C2=O)C(=O)N1]

To a solution of piperidine-4-carboxylic acid (94 mg, 0.7 mmol) and 2-(2,6-dioxo-3-piperidyl)-5-fluoro-isoindoline-1,3-dione (200 mg, 0.7 mmol) in dimethylsulfoxide (2 mL) was added N, N-diisopropylethylamine (468 mg, 4 mmol). The mixture was stirred at 100° C. for 10 h, then filtered and concentrated. The residue was purified by prep-HPLC (20%-40% acetonitrile in water (0.2% formic acid) over 10 min) to afford 1-[2-(2,6-dioxo-3-piperidyl)-1,3-dioxo-isoindolin-5-yl]piperidine-4-carboxylic acid (250 mg, 89%) as a yellow solid. MS (ESI) m/z: 386.2 [M+H]⁺.

### Step 2: Preparation of 3-[3-[6-[7-[1-[2-(2,6-dioxo-3-piperidyl)-1,3-dioxo-isoindolin-5-yl]piperidine-4-carbonyl]-2,7-diazaspiro[3.5]nonan-2-yl]-1-oxo-4-(trifluoromethyl)isoindolin-2-yl]phenyl]-3-[(4-methyl-1,2,4-triazol-3-yl)methyl]cyclobutanecarbonitrile

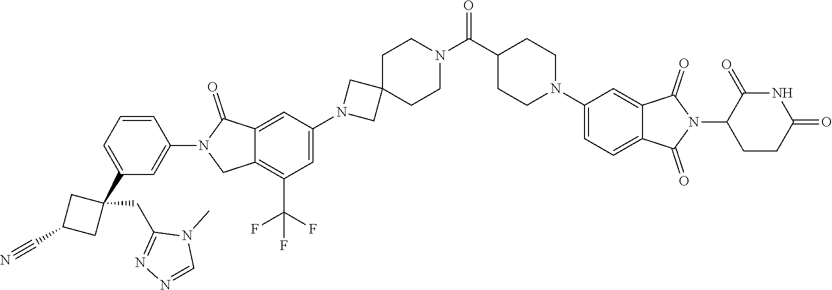 [SMILES: Cn1cnnc1C[C@]1(c2cccc(N3Cc4c(cc(N5CC6(CCN(C(=O)C7CCN(c8ccc9c(c8)C(=O)N(C8CCC(=O)NC8=O)C9=O)CC7)CC6)C5)cc4C(F)(F)F)C3=O)c2)C[C@H](C#N)C1]

To a solution of 1-[2-(2,6-dioxo-3-piperidyl)-1,3-dioxo-isoindolin-5-yl]piperidine-4-carboxylic acid (84 mg, 0.2 mmol) in N, N-dimethylformamide (2 mL) was added N, N-diisopropylethylamine (56 mg, 0.4 mmol), o-(7-azabenzotriazol-1-yl)-n,n,n′,n′-tetramethyluronium hexafluorophosphate (110 mg, 0.3 mmol) and 3-[3-[6-(2,7-diazaspiro[3.5]nonan-2-yl)-1-oxo-4-(trifluoromethyl)isoindolin-2-yl]phenyl]-3-[(4-methyl-1,2,4-triazol-3-yl)methyl]cyclobutanecarbonitrile trifluoroacetate (100 mg, 0.2 mmol). The mixture was stirred for 1 h at 25° C. The crude reaction mixture was purified by prep-HPLC (40%-70% acetonitrile in water (0.2% formic acid) over 10 min) to afford 3-[3-[6-[7-[1-[2-(2,6-dioxo-3-piperidyl)-1,3-dioxo-isoindolin-5-yl]piperidine-4-carbonyl]-2,7-diazaspiro[3.5]nonan-2-yl]-1-oxo-4-(trifluoromethyl)isoindolin-2-yl]phenyl]-3-[(4-methyl-1,2,4-triazol-3-yl)methyl]cyclobutanecarbonitrile (58.4 mg, 41%) as a white solid. MS (ESI) m/z: 944.4 [M+H]⁺; ¹H NMR (400 MHz, DMSO) δ 11.08 (s, 1H), 8.18 (s, 1H), 7.93-7.79 (m, 1H), 7.67 (d, J=8.4 Hz, 1H), 7.44 (s, 1H), 7.38-7.30 (m, 2H), 7.26 (dd, J=1.6, 8.8 Hz, 1H), 6.95 (d, J=4.8 Hz, 2H), 6.82 (d, J=7.6 Hz, 1H), 5.08 (dd, J=5.6, 12.8 Hz, 1H), 4.96 (s, 2H), 4.08 (d, J=13.2 Hz, 2H), 3.78 (d, J=5.2 Hz, 4H), 3.63-3.44 (m, 4H), 3.28-3.23 (m, 1H), 3.18-3.07 (m, 2H), 3.05-2.96 (m, 1H), 2.88 (d, J=8.0 Hz, 4H), 2.80 (s, 3H), 2.61 (d, J=2.8 Hz, 1H), 2.58-2.53 (m, 4H), 2.09-1.97 (m, 1H), 1.83 (s, 2H), 1.77-1.67 (m, 4H), 1.63 (d, J=12.0 Hz, 2H).

### Example 147: Exemplary synthesis of 3-[4-fluoro-5-[4-[4-[[4-[[(3S)-1-[[2-[3-[3-[(4-methyl-1,2,4-triazol-3-yl)methyl]oxetan-3-yl]phenyl]indazol-6-yl]methyl]-3-piperidyl]methyl]piperazin-1-yl]methyl]cyclohexoxy]-1-piperidyl]-1-oxo-isoindolin-2-yl]piperidine-2,6-dione

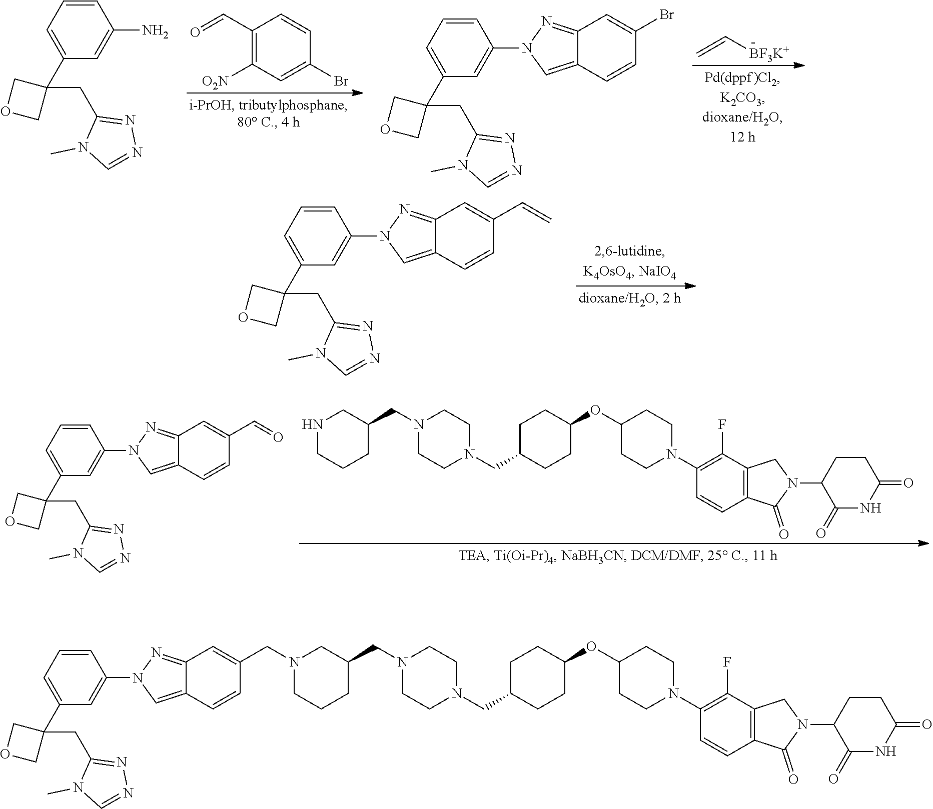

### Step 1: Preparation of 6-bromo-2-[3-[3-[(4-methyl-1,2,4-triazol-3-yl)methyl]oxetan-3-yl]phenyl]indazole

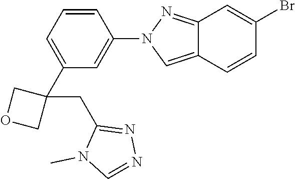 [SMILES: Cn1cnnc1CC1(c2cccc(-n3cc4ccc(Br)cc4n3)c2)COC1]

A mixture of 4-bromo-2-nitro-benzaldehyde (94 mg, 0.4 mmol), 3-[3-[(4-methyl-1,2,4-triazol-3-yl)methyl]oxetan-3-yl]aniline (100 mg, 0.4 mmol) and tributylphosphine (248 mg, 1.2 mmol) in isopropanol (5 mL) was purged with nitrogen three times, then the mixture was stirred at 80° C. for 4 h. The reaction was partitioned between ethyl acetate (100 mL) and water (100 mL). The organic phase was separated, washed with brine (30 mL×3), dried over sodium sulfate, filtered, and concentrated under reduced pressure. The residue was purified by prep-TLC (dichloromethane:methanol=15:1) to afford 6-bromo-2-[3-[3-[(4-methyl-1,2,4-triazol-3-yl)methyl]oxetan-3-yl]phenyl]indazole (40 mg, 23%) as a white solid. ¹H NMR (400 MHz, CDCl₃) δ 8.23 (s, JH), 7.87-7.77 (m, 2H), 7.50 (d, J=8.8 Hz, JH), 7.39-7.31 (m, 2H), 7.12 (d, J=8.8 Hz, 1H), 6.80 (d, J=7.6 Hz, 1H), 5.23 (s, 2H), 5.10-5.04 (m, 3H), 3.57-3.47 (m, 2H), 2.85 (s, 3H).

### Step 2: Preparation of 2-[3-[3-[(4-methyl-1,2,4-triazol-3-yl)methyl]oxetan-3-yl]phenyl]-6-vinyl-indazole

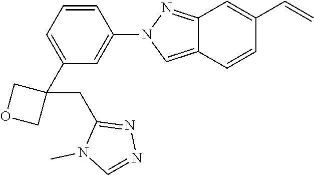 [SMILES: C=Cc1ccc2cn(-c3cccc(C4(Cc5nncn5C)COC4)c3)nc2c1]

A mixture of 6-bromo-2-[3-[3-[(4-methyl-1,2,4-triazol-3-yl)methyl]oxetan-3-yl]phenyl]indazole (300 mg, 0.7 mmol), potassium; trifluoro(vinyl)boranuide (189 mg, 1.4 mmol), potassium carbonate (293 mg, 2 mmol), [1,1′-bis(diphenylphosphino)ferrocene]dichloropalladium(II) (51 mg, 0.07 mmol) in 1,4-dioxane (6 mL) and water (1.5 mL) was degassed and purged with nitrogen three times. The mixture was allowed to stir at 90° C. for 12 h, then partitioned between ethyl acetate (30 mL) and water (30 mL). The organic phase was separated, washed with brine (20 mL), dried over sodium sulfate, filtered, and concentrated under reduced pressure. The residue was purified by prep-TLC (dichloromethane:methanol=15:1) to afford 2-[3-[3-[(4-methyl-1,2,4-triazol-3-yl) methyl]oxetan-3-yl]phenyl]-6-vinyl-indazole (280 mg, crude) as a yellow oil. ¹H NMR (400 MHz, CDCl₃) δ 8.20 (s, 1H), 7.94 (s, 1H), 7.81 (s, 1H), 7.71 (dd, J=1.6, 8.0 Hz, 1H), 7.60-7.55 (m, 2H), 6.82-6.72 (m, 2H), 5.76 (d, J=17.6 Hz, 1H), 5.29-5.21 (m, 3H), 5.11-5.02 (m, 4H), 3.53 (s, 2H), 3.41 (s, 1H), 1.83 (s, 2H).

### Step 3: Preparation of 2-[3-[3-[(4-methyl-1,2,4-triazol-3-yl)methyl]oxetan-3-yl]phenyl]indazole-6-carbaldehyde

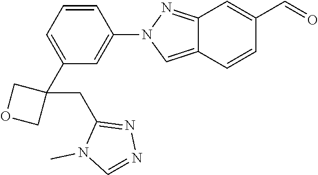 [SMILES: Cn1cnnc1CC1(c2cccc(-n3cc4ccc(C=O)cc4n3)c2)COC1]

To a solution of 2-[3-[3-[(4-methyl-1,2,4-triazol-3-yl)methyl]oxetan-3-yl]phenyl]-6-vinyl-indazole (280 mg, 0.7 mmol) and 2,6-dimethylpyridine (0.1 mL, 1 mmol) in 1,4-dioxane (25 mL) and water (7 mL) was added potassium osmate dihydrate (28 mg, 0.07 mmol) and sodium periodate (645 mg, 3 mmol). The mixture was stirred at 20° C. for 2 h. The mixture was filtered and the filtrate was diluted with saturated aqueous sodium thiosulfate solution (100 mL) and extracted with ethyl acetate (100 mL×3). The combined organic layers were washed with brine (50 mL), dried over sodium sulfate, filtered, and concentrated under reduced pressure. The residue was purified by prep-TLC (dichloromethane:methanol=15:1) to afford 2-[3-[3-[(4-methyl-1,2,4-triazol-3-yl)methyl]oxetan-3-yl]phenyl]indazole-6-carbaldehyde (100 mg, 35%) as a colorless oil. MS (ESI) m/z: 374.3 [M+H]⁺.

### Step 4: Preparation of 3-[4-fluoro-5-[4-[4-[[4-[[(3S)-1-[[2-[3-[3-[(4-methyl-1,2,4-triazol-3-yl)methyl]oxetan-3-yl]phenyl]indazol-6-yl]methyl]-3-piperidyl]methyl]piperazin-1-yl]methyl]cyclohexoxy]-1-piperidyl]-1-oxo-isoindolin-2-yl]piperidine-2,6-dione

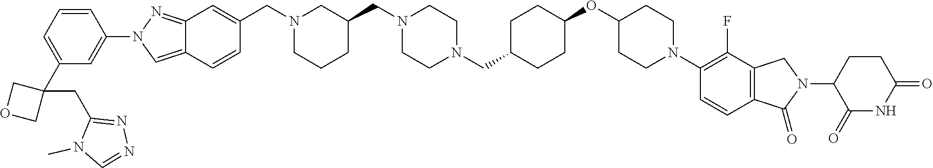 [SMILES: Cn1cnnc1CC1(c2cccc(-n3cc4ccc(CN5CCC[C@H](CN6CCN(C[C@H]7CC[C@H](OC8CCN(c9ccc%10c(c9F)CN(C9CCC(=O)NC9=O)C%10=O)CC8)CC7)CC6)C5)cc4n3)c2)COC1]

To a solution of 2-[3-[3-[(4-methyl-1,2,4-triazol-3-yl)methyl]oxetan-3-yl]phenyl]indazole-6-carbaldehyde (200 mg, 0.5 mmol) and 3-[4-fluoro-1-oxo-5-[4-[4-[[4-[[(3S)-3-piperidyl]methyl]piperazin-1-yl]methyl]cyclohexoxy]-1-piperidyl]isoindolin-2-yl]piperidine-2,6-dione trifluoroacetate (443 mg, 0.6 mmol) in dichloromethane (2 mL) and N, N-dimethylformamide (2 mL) was added triethylamine (163 mg, 2 mmol) and titanium(IV) isopropoxide (152 mg, 0.5 mmol), followed by the addition of sodium cyanoborohydride (101 mg, 1.6 mmol). The mixture was stirred at 25° C. for 10 h, then concentrated under reduced pressure. The residue was purified by prep-HPLC (5%-35% acetonitrile in water (0.2% formic acid) over 15 min) to afford 3-[4-fluoro-5-[4-[4-[[4-[[(3S)-1-[[2-[3-[3-[(4-methyl-1,2,4-triazol-3-yl)methyl]oxetan-3-yl]phenyl]indazol-6-yl]methyl]-3-piperidyl]methyl]piperazin-1-yl]methyl]cyclohexoxy]-1-piperidyl]-1-oxo-isoindolin-2-yl]piperidine-2,6-dione diformate (108.1 mg, 18%) as an off-white solid. MS (ESI) m/z: 499.0 [M/2+H]⁺; ¹H NMR (400 MHz, DMSO-d₆) δ 10.98 (s, 1H), 9.03 (s, 1H), 8.21 (s, 1H), 8.17 (s, 2H), 7.94 (dd, J=1.4, 8.2 Hz, 1H), 7.77 (s, 1H), 7.71 (d, J=8.8 Hz, 1H), 7.57 (s, 1H), 7.52-7.41 (m, 2H), 7.16 (t, J=8.0 Hz, 1H), 7.09 (d, J=8.8 Hz, 1H), 7.01 (d, J=7.6 Hz, 1H), 5.08 (dd, J=5.2, 13.2 Hz, 1H), 5.03-4.97 (m, 2H), 4.97-4.90 (m, 2H), 4.48 (d, J=17.2 Hz, 1H), 4.31 (d, J=16.8 Hz, 1H), 3.66-3.60 (m, 2H), 3.58 (s, 2H), 3.56 (s, 2H), 3.35-3.26 (m, 2H), 3.01 (s, 3H), 2.98-2.89 (m, 3H), 2.83 (s, 1H), 2.78-2.71 (m, 1H), 2.62-2.57 (m, 1H), 2.54 (d, J=6.8 Hz, 2H), 2.47 (d, J=3.2 Hz, 1H), 2.42 (dd, J=4.8, 13.2 Hz, 2H), 2.30 (dd, J=2.0, 4.8 Hz, 4H), 2.13 (s, 2H), 2.07-1.96 (m, 4H), 1.91 (d, J=10.0 Hz, 4H), 1.79 (d, J=5.2 Hz, 2H), 1.71 (d, J=12.4 Hz, 3H), 1.62-1.51 (m, 3H), 1.51-1.43 (m, 1H), 1.42-1.33 (m, 1H), 1.19-1.07 (m, 2H), 0.96-0.77 (m, 3H).

### Example 148: Exemplary synthesis of 3-[5-[4-[4-[[3,3-dimethyl-4-[[(3S)-1-[[2-[3-[3-[(4-methyl-1,2,4-triazol-3-yl)methyl]oxetan-3-yl]phenyl]-3-oxo-7-(trifluoromethyl)isoindolin-5-yl]methyl]-3-piperidyl]methyl]piperazin-1-yl]methyl]cyclohexoxy]-1-piperidyl]-4-fluoro-1-oxo-isoindolin-2-yl]piperidine-2,6-dione

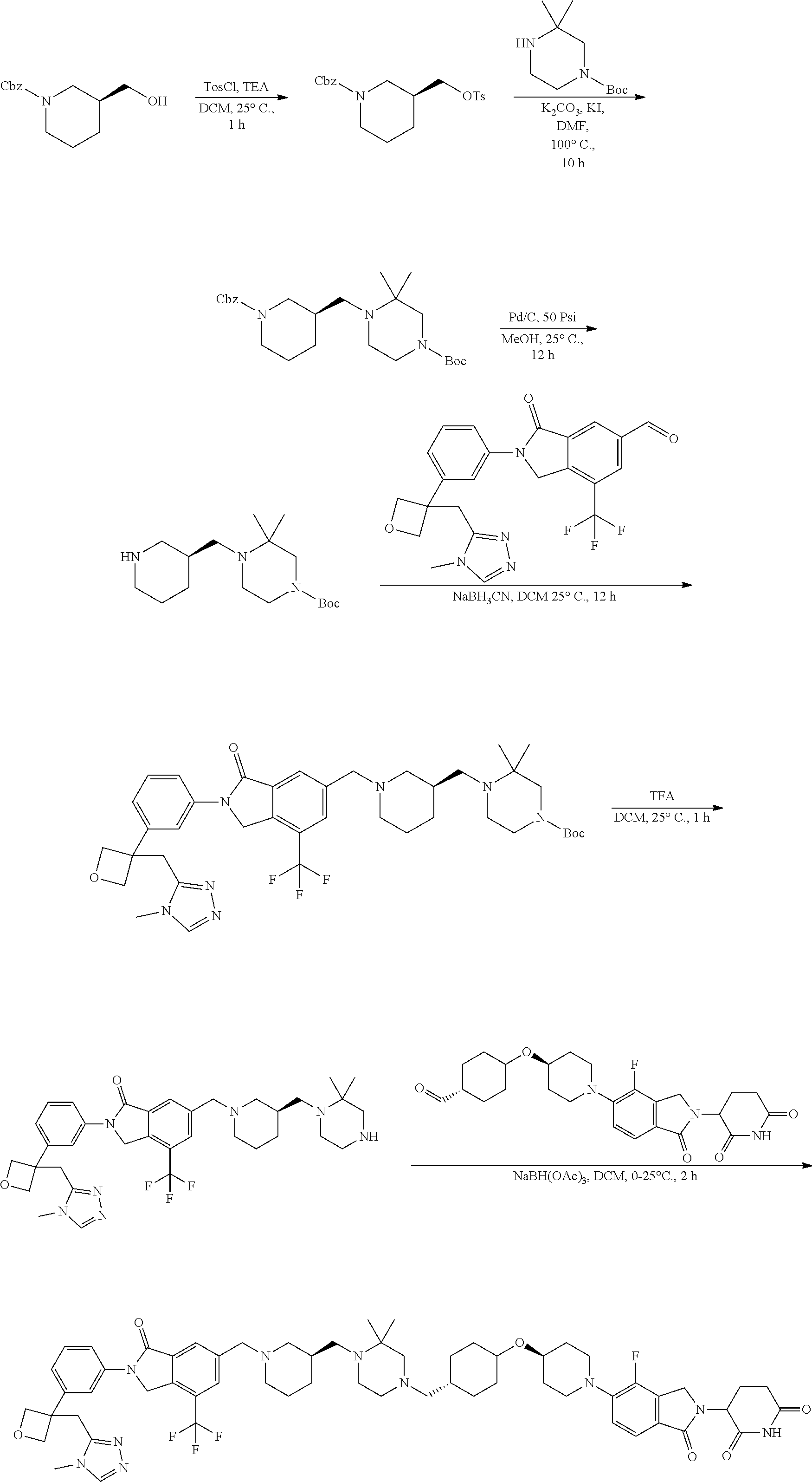

### Step 1: Preparation of benzyl (3S)-3-(p-tolylsulfonyloxymethyl)piperidine-1-carboxylate

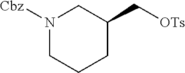 [SMILES: Cc1ccc(S(=O)(=O)OC[C@H]2CCCN(C(=O)OCc3ccccc3)C2)cc1]

To a solution of benzyl (3S)-3-(hydroxymethyl)piperidine-1-carboxylate (8.90 g, 36 mmol) in dichloromethane (90 mL) was added triethylamine (10.84 g, 107 mmol) and p-toluenesulfonyl chloride (13.61 g, 71 mmol). The mixture was stirred at 25° C. for 1 h, then concentrated under reduced pressure. The residue was purified by column chromatography on silica gel (petroleum ether:ethyl acetate=50:1 to 3:1) to afford benzyl (3S)-3-(p-tolylsulfonyloxymethyl)piperidine-1-carboxylate (13.00 g, 85%) as a colorless oil. MS (ESI) m/z: 404.1 [M+H]⁺; ¹H NMR (400 MHz, CDCl₃) δ 7.78 (d, J=7.6 Hz, 2H), 7.39-7.31 (m, 7H), 5.12 (s, 2H), 4.03-3.85 (m, 4H), 2.92-2.82 (m, 1H), 2.79-2.59 (m, 1H), 2.45 (s, 3H), 1.93-1.83 (m, 1H), 1.81-1.73 (m, 1H), 1.68-1.62 (m, 1H), 1.48-1.42 (m, 1H), 1.27 (t, J=7.2 Hz, 1H).

### Step 2: Preparation of tert-butyl 4-[[(3R)-1-benzyloxycarbonyl-3-piperidyl]methyl]-3,3-dimethyl-piperazine-1-carboxylate

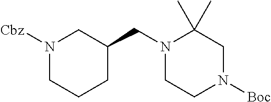 [SMILES: CC(C)(C)OC(=O)N1CCN(C[C@H]2CCCN(C(=O)OCc3ccccc3)C2)C(C)(C)C1]

To a solution of benzyl (3S)-3-(p-tolylsulfonyloxymethyl)piperidine-1-carboxylate (3.00 g, 7.4 mmol), potassium iodide (617 mg, 3.7 mmol) and tert-butyl 3,3-dimethylpiperazine-1-carboxylate (1.75 g, 8 mmol) in N, N-dimethylformamide (20 mL) was added potassium carbonate (3.08 g, 22 mmol). The mixture was stirred at 100° C. for 10 h, then concentrated under reduced pressure. The residue was purified by column chromatography (petroleum ether:ethyl acetate=50:1 to 15:1) to afford tert-butyl 4-[[(3R)-1-benzyloxycarbonyl-3-piperidyl]methyl]-3,3-dimethyl-piperazine-1-carboxylate (1.10 g, 33%) as a yellow solid. ¹H NMR (400 MHz, CDCl₃) δ 7.27 (s, 5H), 5.12-4.97 (m, 2H), 4.13-4.02 (m, 1H), 4.03-3.98 (m, 1H), 3.42 (d, J=4.0 Hz, 1H), 3.17-3.10 (m, 1H), 2.99-2.86 (m, 1H), 2.82-2.70 (m, 1H), 2.48-2.24 (m, 3H), 2.16-2.06 (m, 1H), 2.02-1.95 (m, 1H), 1.79-1.69 (m, 1H), 1.60-1.44 (m, 3H), 1.37 (s, 9H), 1.23-1.15 (m, 1H), 1.03-0.92 (m, 1H), 0.85 (d, J=8.4 Hz, 6H).

### Step 3: Preparation of tert-butyl 3,3-dimethyl-4-[[(3S)-3-piperidyl]methyl]piperazine-1-carboxylate

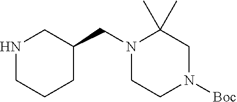 [SMILES: CC(C)(C)OC(=O)N1CCN(C[C@H]2CCCNC2)C(C)(C)C1]

To a solution of tert-butyl 4-[[(3R)-1-benzyloxycarbonyl-3-piperidyl]methyl]-3,3-dimethyl-piperazine-1-carboxylate (400 mg, 0.9 mmol) in methanol (10 mL) was added 10% palladium on carbon (100 mg) under nitrogen. The suspension was degassed and purged with hydrogen several times before stirring under hydrogen (50 psi) at 25° C. for 12 h. The mixture was filtered through Celite pad and the filtrate was concentrated under reduced pressure to afford tert-butyl 3,3-dimethyl-4-[[(3S)-3-piperidyl]methyl]piperazine-1-carboxylate (250 mg, 89%) was colorless oil. ¹H NMR (400 MHz, CDCl₃) δ 3.58-3.46 (m, 1H), 3.31-3.15 (m, 3H), 3.10-2.98 (m, 2H), 2.61-2.49 (m, 2H), 2.44-2.37 (m, 1H), 2.25-2.12 (m, 2H), 2.10-2.03 (m, 1H), 1.82 (d, J=12.4 Hz, 1H), 1.73-1.50 (m, 3H), 1.46 (s, 9H), 1.31-1.24 (m, 1H), 0.94 (d, J=7.6 Hz, 7H).

### Step 4: Preparation of tert-butyl (R)-3,3-dimethyl-4-((1-((2-(3-(3-((4-methyl-4H-1,2,4-triazol-3-yl)methyl)oxetan-3-yl)phenyl)-3-oxo-7-(trifluoromethyl)isoindolin-5-yl)methyl)piperidin-3-yl)methyl)piperazine-1-carboxylate

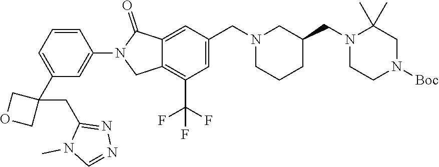 [SMILES: Cn1cnnc1CC1(c2cccc(N3Cc4c(cc(CN5CCC[C@H](CN6CCN(C(=O)OC(C)(C)C)CC6(C)C)C5)cc4C(F)(F)F)C3=O)c2)COC1]

A solution of tert-butyl 3,3-dimethyl-4-[[(3S)-3-piperidyl]methyl]piperazine-1-carboxylate (220 mg, 0.7 mmol) and 2-(3-(3-((4-methyl-4H-1,2,4-triazol-3-yl)methyl)oxetan-3-yl)phenyl)-3-oxo-7-(trifluoromethyl)isoindoline-5-carbaldehyde (322 mg, 0.7 mmol) in methanol (3 mL) was allowed to stir at 25° C. for 11 h. Then sodium cyanoborohydride (178 mg, 2.8 mmol) was added, the mixture was stirred at 25° C. for 1 h. The reaction was concentrated under reduced pressure. The residue was purified by prep-TLC (dichloromethane:methanol=10:1) to afford tert-butyl (R)-3,3-dimethyl-4-((1-((2-(3-(3-((4-methyl-4H-1,2,4-triazol-3-yl)methyl)oxetan-3-yl)phenyl)-3-oxo-7-(trifluoromethyl)isoindolin-5-yl)methyl)piperidin-3-yl)methyl)piperazine-1-carboxylate (200 mg, 36%) as a colorless oil. MS (ESI) m/z: 752.6 [M+H]⁺.

### Step 5: Preparation of 6-[[(3S)-3-[(2,2-dimethylpiperazin-1-yl)methyl]-1-piperidyl]methyl]-2-[3-[3-[(4-methyl-1,2,4-triazol-3-yl)methyl]oxetan-3-yl]phenyl]-4-(trifluoromethyl)isoindolin-1-one

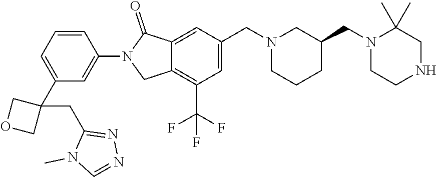 [SMILES: Cn1cnnc1CC1(c2cccc(N3Cc4c(cc(CN5CCC[C@H](CN6CCNCC6(C)C)C5)cc4C(F)(F)F)C3=O)c2)COC1]

To a solution of tert-butyl 3,3-dimethyl-4-[[(3S)-1-[[2-[3-[3-[(4-methyl-1,2,4-triazol-3-yl)methyl]oxetan-3-yl]phenyl]-3-oxo-7-(trifluoromethyl)isoindolin-5-yl]methyl]-3-piperidyl]methyl]piperazine-1-carboxylate (120 mg, 0.2 mmol) in dichloromethane (2 mL) was added trifluoroacetic acid (768 mg). The mixture was stirred at 25° C. for 1 h, then concentrated under reduced pressure. The residue was purified by prep-HPLC (1%-30% acetonitrile in water (0.2% formic acid) over 10 min) to afford 6-[[(3S)-3-[(2,2-dimethylpiperazin-1-yl)methyl]-1-piperidyl]methyl]-2-[3-[3-[(4-methyl-1,2,4-triazol-3-yl)methyl]oxetan-3-yl]phenyl]-4-(trifluoromethyl)isoindolin-1-one formate (100 mg, 90%) as a white solid. MS (ESI) m/z: 652.5 [M+H]⁺.

### Step 6: Preparation of 3-[5-[4-[4-[[3,3-dimethyl-4-[[(3S)-1-[[2-[3-[3-[(4-methyl-1,2,4-triazol-3-yl)methyl]oxetan-3-yl]phenyl]-3-oxo-7-(trifluoromethyl)isoindolin-5-yl]methyl]-3-piperidyl]methyl]piperazin-1-yl]methyl]cyclohexoxy]-1-piperidyl]-4-fluoro-1-oxo-isoindolin-2-yl]piperidine-2,6-dione

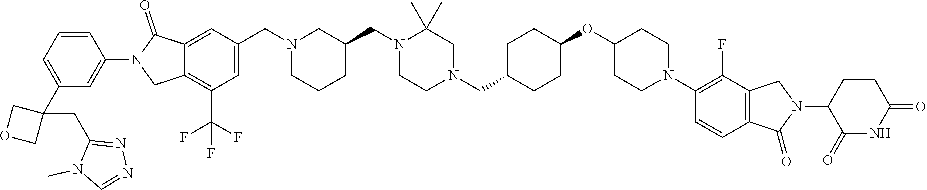 [SMILES: Cn1cnnc1CC1(c2cccc(N3Cc4c(cc(CN5CCC[C@H](CN6CCN(C[C@H]7CC[C@H](OC8CCN(c9ccc%10c(c9F)CN(C9CCC(=O)NC9=O)C%10=O)CC8)CC7)CC6(C)C)C5)cc4C(F)(F)F)C3=O)c2)COC1]

To a stirred solution of 6-[[(3S)-3-[(2,2-dimethylpiperazin-1-yl)methyl]-1-piperidyl]methyl]-2-[3-[3-[(4-methyl-1,2,4-triazol-3-yl)methyl]oxetan-3-yl]phenyl]-4-(trifluoromethyl)isoindolin-1-one formate (90 mg, 0.1 mmol) in dichloromethane (2 mL) was added 4-methylmorpholine (26 mg, 0.3 mmol) and 4-[[1-[2-(2,6-dioxo-3-piperidyl)-4-fluoro-1-oxo-isoindolin-5-yl]-4-piperidyl]oxy]cyclohexanecarbaldehyde (61 mg, 0.1 mmol). The mixture was stirred at 0° C. for 1 h, then sodium triacetoxyborohydride (55 mg, 0.3 mmol) was added and the mixture was stirred at 25° C. for 1 h. The reaction mixture was concentrated under reduced pressure. The residue was purified by prep-HPLC (5%-35% acetonitrile in water (0.2% formic acid) over 10 min) to afford 3-[5-[4-[4-[[3,3-dimethyl-4-[[(3S)-1-[[2-[3-[3-[(4-methyl-1,2,4-triazol-3-yl)methyl]oxetan-3-yl]phenyl]-3-oxo-7-(trifluoromethyl)isoindolin-5-yl]methyl]-3-piperidyl]methyl]piperazin-1-yl]methyl]cyclohexoxy]-1-piperidyl]-4-fluoro-1-oxo-isoindolin-2-yl]piperidine-2,6-dione formate (85.7 mg, 55%) as a white solid. MS (ESI) m/z: 1108.3 [M+H]⁺; ¹H NMR (400 MHz, DMSO-d₆) δ 10.97 (s, 1H), 8.18 (s, 1H), 8.13 (s, 1H), 8.04 (s, 1H), 7.98 (s, 1H), 7.88 (d, J=9.6 Hz, 1H), 7.45 (d, J=8.0 Hz, 1H), 7.40 (s, 1H), 7.35 (t, J=8.0 Hz, 1H), 7.16 (t, J=8.0 Hz, 1H), 6.79 (d, J=7.6 Hz, 1H), 5.12 (s, 2H), 5.08-5.03 (m, 1H), 4.96 (d, J=6.0 Hz, 2H), 4.88 (d, J=6.0 Hz, 2H), 4.50-4.43 (m, 1H), 4.35-4.25 (m, 1H), 3.60 (d, J=7.6, 4.0 Hz, 1H), 3.51 (s, 2H), 3.24-3.14 (m, 2H), 2.98-2.83 (m, 8H), 2.63-2.54 (m, 3H), 2.52 (s, 4H), 2.46-2.32 (m, 4H), 2.08-1.86 (m, 9H), 1.78-1.60 (m, 5H), 1.60-1.35 (m, 5H), 1.23-0.97 (m, 9H), 0.97-0.79 (m, 3H).

### Example 149: Exemplary synthesis of 3-[4-methoxy-5-[4-[[2-[2-[3-[3-[(4-methyl-1,2,4-triazol-3-yl)methyl]oxetan-3-yl]phenyl]-3-oxo-7-(trifluoromethyl)isoindolin-5-yl]-2-azaspiro[3.5]nonan-7-yl]oxy]-1-piperidyl]-1-oxo-isoindolin-2-yl]piperidine-2,6-dione

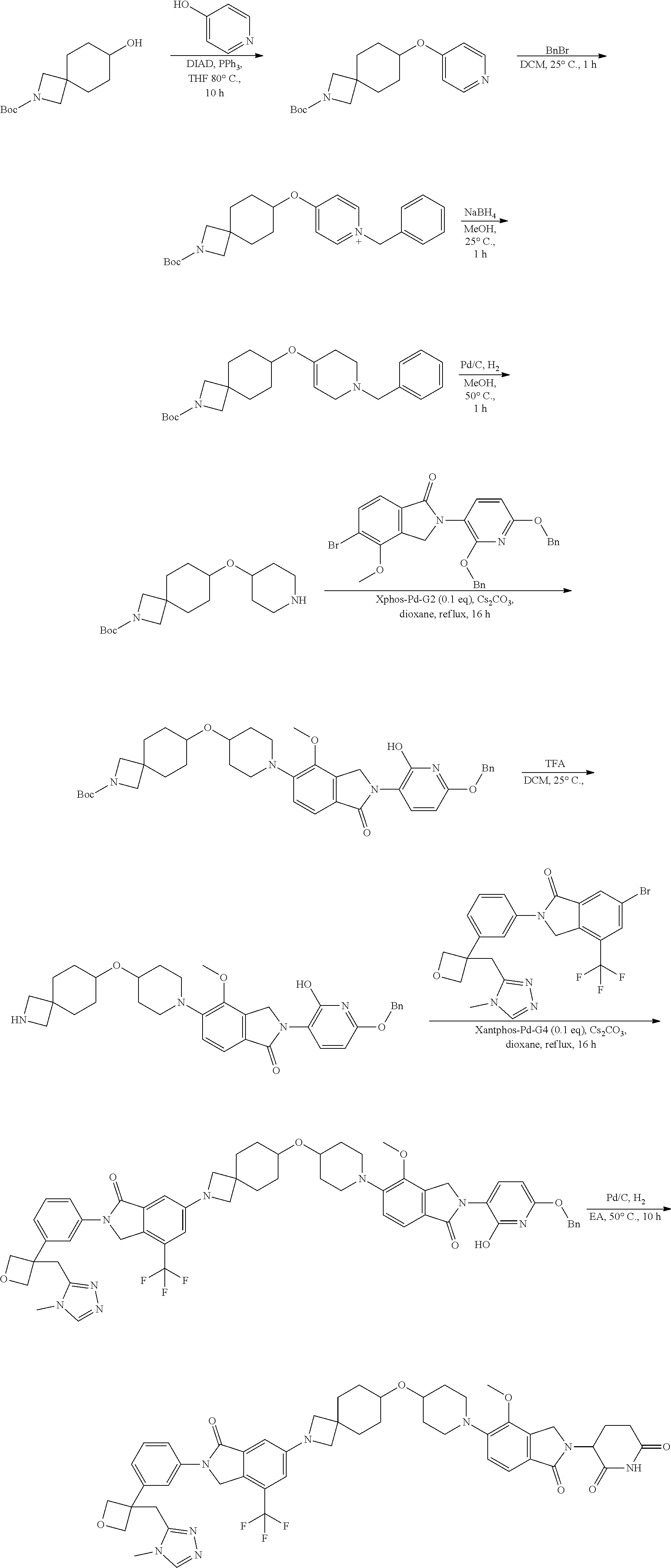

### Step 1: Preparation of tert-butyl 7-(4-pyridyloxy)-2-azaspiro[3.5]nonane-2-carboxylate

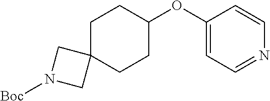 [SMILES: CC(C)(C)OC(=O)N1CC2(CCC(Oc3ccncc3)CC2)C1]

To a solution of tert-butyl 7-hydroxy-2-azaspiro[3.5]nonane-2-carboxylate (1.95 g, 8 mmol) and pyridin-4-ol (700 mg, 7 mmol) in tetrahydrofuran (5 ml.) was added triphenylphosphine (2.70 g, 10 mmol) and diisopropyl azodicarboxylate (2.08 g, 10 mmol) under nitrogen. The mixture was stirred at 80° C. for 10 h under nitrogen, then concentrated under reduced pressure. The residue was purified by silica gel column chromatography (petroleum ether/ethyl acetate=10/1 to 1/1) to afford tert-butyl 7-(4-pyridyloxy)-2-azaspiro[3.5]nonane-2-carboxylate (1.40 g, 60%) as a white solid. MS (ESI) m/z: 319.3 [M+H]⁺.

### Step 2: Preparation of tert-butyl 7-(1-benzylpyridin-1-ium-4-yl)oxy-2-azaspiro[3.5]nonane-2-carboxylate

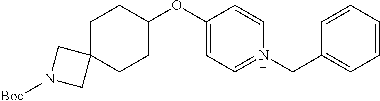

To a solution of tert-butyl 7-(4-pyridyloxy)-2-azaspiro[3.5]nonane-2-carboxylate (1.40 g, 4 mmol) in dichloromethane (10 mL) was added benzyl bromide (1.13 g, 6.6 mmol). The mixture was stirred at 25° C. for 1 h, then concentrated under reduced pressure. The residue was purified by re-crystallization from ethyl acetate (30 mL) to afford tert-butyl 7-(1-benzylpyridin-1-ium-4-yl)oxy-2-azaspiro[3.5]nonane-2-carboxylate (1.15 g, 64%) as white solid. MS (ESI) m/z: 409.2 [M+H]⁺.

### Step 3: Preparation of tert-butyl 7-[(1-benzyl-3,6-dihydro-2H-pyridin-4-yl)oxy]-2-azaspiro[3.5]nonane-2-carboxylate

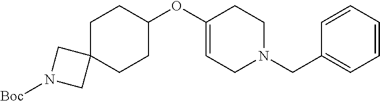 [SMILES: CC(C)(C)OC(=O)N1CC2(CCC(OC3=CCN(Cc4ccccc4)CC3)CC2)C1]

To a solution of tert-butyl 7-(1-benzylpyridin-1-ium-4-yl)oxy-2-azaspiro[3.5]nonane-2-carboxylate (1.50 g, 3.7 mmol) in methanol (15 mL) was added sodium borohydride (1.11 g, 29 mmol). The mixture was stirred at 25° C. for 1 h, then concentrated under reduced pressure. The residue was purified by silica gel column chromatography (petroleum ether/ethyl acetate=10/1 to 3/1) to afford tert-butyl 7-[(1-benzyl-3,6-dihydro-2H-pyridin-4-yl)oxy]-2-azaspiro[3.5]nonane-2-carboxylate (1.10 g, 73%) as a white solid. MS (ESI) m/z: 413.3 [M+H]⁺.

### Step 4: Preparation of tert-butyl 7-(4-piperidyloxy)-2-azaspiro[3.5]nonane-2-carboxylate

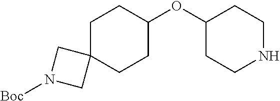 [SMILES: CC(C)(C)OC(=O)N1CC2(CCC(OC3CCNCC3)CC2)C1]

To a solution of tert-butyl 7-[(1-benzyl-3,6-dihydro-2H-pyridin-4-yl)oxy]-2-azaspiro[3.5]nonane-2-carboxylate (1.10 g, 2.7 mmol) in methanol (10 mL) was added 10% palladium on carbon (100 mg) at 25° C. under nitrogen. The suspension was degassed and purged with hydrogen several times. The mixture was stirred at 50° C. under hydrogen (50 psi) for 10 h, then filtered and concentrated under reduced pressure to afford tert-butyl 7-(4-piperidyloxy)-2-azaspiro[3.5]nonane-2-carboxylate (700 mg, 81%) as a white solid. MS (ESI) m/z: 325.3 [M+H]⁺.

### Step 5: Preparation of tert-butyl 7-[[1-[2-(2,6-dibenzyloxy-3-pyridyl)-4-methoxy-1-oxo-isoindolin-5-yl]-4-piperidyl]oxy]-2-azaspiro[3.5]nonane-2-carboxylate

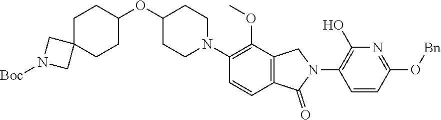 [SMILES: COc1c(N2CCC(OC3CCC4(CC3)CN(C(=O)OC(C)(C)C)C4)CC2)ccc2c1CN(c1ccc(OCc3ccccc3)nc1O)C2=O]

To a solution of 5-bromo-2-(2,6-dibenzyloxy-3-pyridyl)-4-methoxy-isoindolin-1-one (550 mg, 1 mmol) and tert-butyl 7-(4-piperidyloxy)-2-azaspiro[3.5]nonane-2-carboxylate (403 mg, 1 mmol) in 1,4-dioxane (5 mL) was added cesium carbonate (1.01 g, 3 mmol) and [2-(2-aminophenyl)phenyl]-chloro-palladium; dicyclohexyl-[3-(2,4,6-triisopropylphenyl)phenyl]phosphane (163 mg, 0.2 mmol). The mixture was stirred at 100° C. for 10 h under nitrogen, then concentrated under reduced pressure. The residue was purified by silica gel column chromatography (petroleum ether/ethyl acetate=10/1 to 3/1) to afford tert-butyl 7-[[1-[2-(2,6-dibenzyloxy-3-pyridyl)-4-methoxy-1-oxo-isoindolin-5-yl]-4-piperidyl]oxy]-2-azaspiro[3.5]nonane-2-carboxylate (220 mg, 27%) as a white solid. MS (ESI) m/z: 775.5 [M+H]⁺.

### Step 6: Preparation of 5-[4-(2-azaspiro[3.5]nonan-7-yloxy)-1-piperidyl]-2-(6-benzyloxy-2-hydroxy-3-pyridyl)-4-methoxy-isoindolin-1-one

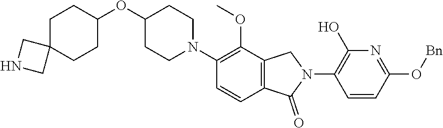 [SMILES: COc1c(N2CCC(OC3CCC4(CC3)CNC4)CC2)ccc2c1CN(c1ccc(OCc3ccccc3)nc1O)C2=O]

To a solution of tert-butyl 7-[[1-[2-(2,6-dibenzyloxy-3-pyridyl)-4-methoxy-1-oxo-isoindolin-5-yl]-4-piperidyl]oxy]-2-azaspiro[3.5]nonane-2-carboxylate (200 mg, 0.3 mmol) in dichloromethane (2 mL) was added trifluoroacetic acid (147 mg, 1.3 mmol). The mixture was stirred at 25° C. for 10 min, then concentrated under reduced pressure to afford 5-[4-(2-azaspiro[3.5]nonan-7-yloxy)-1-piperidyl]-2-(6-benzyloxy-2-hydroxy-3-pyridyl)-4-methoxy-isoindolin-1-one trifluoroacetate (181 mg, crude) as a yellow oil. MS (ESI) m/z: 585.4 [M+H]⁺.

### Step 7: Preparation of 2-(6-benzyloxy-2-hydroxy-3-pyridyl)-4-methoxy-5-[4-[[2-[2-[3-[3-[(4-methyl-1,2,4-triazol-3-yl)methyl]oxetan-3-yl]phenyl]-3-oxo-7-(trifluoromethyl)isoindolin-5-yl]-2-azaspiro[3.5]nonan-7-yl]oxy]-1-piperidyl]isoindolin-1-one

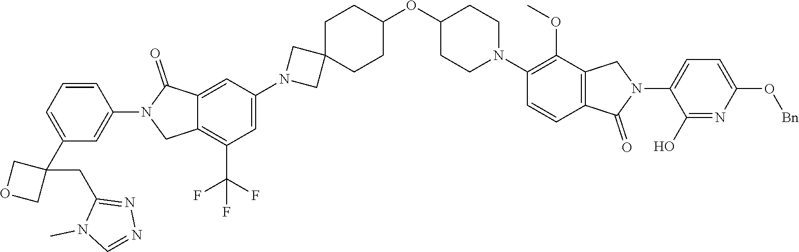 [SMILES: COc1c(N2CCC(OC3CCC4(CC3)CN(c3cc5c(c(C(F)(F)F)c3)CN(c3cccc(C6(Cc7nncn7C)COC6)c3)C5=O)C4)CC2)ccc2c1CN(c1ccc(OCc3ccccc3)nc1O)C2=O]

To a solution of 5-[4-(2-azaspiro[3.5]nonan-7-yloxy)-1-piperidyl]-2-(6-benzyloxy-2-hydroxy-3-pyridyl)-4-methoxy-isoindolin-1-one trifluoroacetate (180 mg, 0.3 mmol) and 6-bromo-2-[3-[3-[(4-methyl-1,2,4-triazol-3-yl)methyl]oxetan-3-yl]phenyl]-4-(trifluoromethyl)isoindolin-1-one (131 mg, 0.3 mmol) in 1,4-dioxane (2 mL) was added cesium carbonate (252 mg, 0.8 mmol) and chloro[(4,5-bis(diphenylphosphino)-9,9-dimethylxanthene)-2-(2′-amino-1,1′-biphenyl)]palladium(II) (22 mg, 0.03 mmol). The mixture was stirred at 90° C. for 10 h under nitrogen, then concentrated under reduced pressure. The residue was purified by prep-HPLC (50%-80% acetonitrile in water (0.2% formic acid) over 10 min) to afford 2-(6-benzyloxy-2-hydroxy-3-pyridyl)-4-methoxy-5-[4-[[2-[2-[3-[3-[(4-methyl-1,2,4-triazol-3-yl)methyl]oxetan-3-yl]phenyl]-3-oxo-7-(trifluoromethyl)isoindolin-5-yl]-2-azaspiro[3.5]nonan-7-yl]oxy]-1-piperidyl]isoindolin-1-one (70 mg, 27%) as a yellow solid. MS (ESI) m/z: 506.5 [M/2+H]⁺.

### Step 8: Preparation of 3-[4-methoxy-5-[4-[[2-[2-[3-[3-[(4-methyl-1,2,4-triazol-3-yl)methyl]oxetan-3-yl]phenyl]-3-oxo-7-(trifluoromethyl)isoindolin-5-yl]-2-azaspiro[3.5]nonan-7-yl]oxy]-1-piperidyl]-1-oxo-isoindolin-2-yl]piperidine-2,6-dione

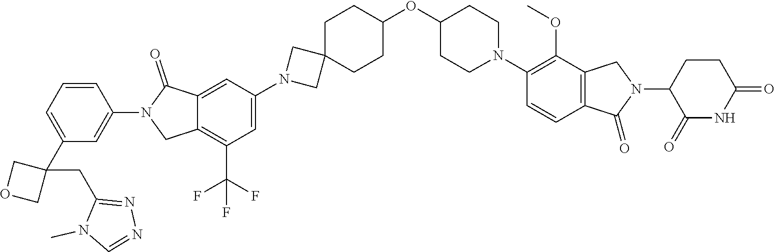 [SMILES: COc1c(N2CCC(OC3CCC4(CC3)CN(c3cc5c(c(C(F)(F)F)c3)CN(c3cccc(C6(Cc7nncn7C)COC6)c3)C5=O)C4)CC2)ccc2c1CN(C1CCC(=O)NC1=O)C2=O]

To a solution of 2-(6-benzyloxy-2-hydroxy-3-pyridyl)-4-methoxy-5-[4-[[2-[2-[3-[3-[(4-methyl-1,2,4-triazol-3-yl)methyl]oxetan-3-yl]phenyl]-3-oxo-7-(trifluoromethyl)isoindolin-5-yl]-2-azaspiro[3.5]nonan-7-yl]oxy]-1-piperidyl]isoindolin-1-one (60 mg, 0.06 mmol) in ethyl acetate (5 mL) was added 10% palladium on carbon (50 mg) at 25° C. under nitrogen. The suspension was degassed and purged with hydrogen several times, then stirred at 50° C. under hydrogen (50 psi) for 10 h. The mixture was filtered and the filtrate was concentrated under reduced pressure. The residue was purified by prep-HPLC (38%-68% acetonitrile in water (0.2% formic acid) over 10 min) to afford 3-[4-methoxy-5-[4-[[2-[2-[3-[3-[(4-methyl-1,2,4-triazol-3-yl)methyl]oxetan-3-yl]phenyl]-3-oxo-7-(trifluoromethyl)isoindolin-5-yl]-2-azaspiro[3.5]nonan-7-yl]oxy]-1-piperidyl]-1-oxo-isoindolin-2-yl]piperidine-2,6-dione (33.7 mg, 61%) as a white solid. MS (ESI) m/z: 923.2 [M+H]⁺; ¹H NMR (400 MHz, DMSO-d₆) δ 10.96 (s, 1H), 8.19 (s, 1H), 7.91-7.85 (m, 1H), 7.39-7.31 (m, 3H), 7.09 (d, J=8.4 Hz, 1H), 6.92 (d, J=5.6 Hz, 2H), 6.74 (d, J=7.6 Hz, 1H), 5.06 (dd, J=5.2, 13.2 Hz, 1H), 4.98-4.92 (m, 4H), 4.88 (d, J=6.0 Hz, 2H), 4.44 (d, J=17.2 Hz, 1H), 4.26 (d, J=16.8 Hz, 1H), 3.87 (s, 3H), 3.68 (d, J=10.8 Hz, 4H), 3.60 (s, 1H), 3.50 (s, 3H), 3.45-3.37 (m, 3H), 2.91-2.82 (m, 6H), 2.55-2.55 (m, 1H), 2.54 (s, 1H), 2.00-1.88 (m, 5H), 1.82-1.72 (m, 2H), 1.65-1.54 (m, 4H), 1.43-1.33 (m, 2H).

### Example 150: Exemplary synthesis of 3-[3-[6-[7-[[(1R,4R)-5-[2-(2,6-dioxo-3-piperidyl)-4-fluoro-7-methyl-1-oxo-isoindolin-5-yl]-2,5-diazabicyclo [2.2.1]heptan-2-yl]methyl]-2-azaspiro [3.5]nonan-2-yl]-1-oxo-4-(trifluoromethyl)isoindolin-2-yl]phenyl]-3-[(4-methyl-1,2,4-triazol-3-yl)methyl]cyclobutanecarbonitrile

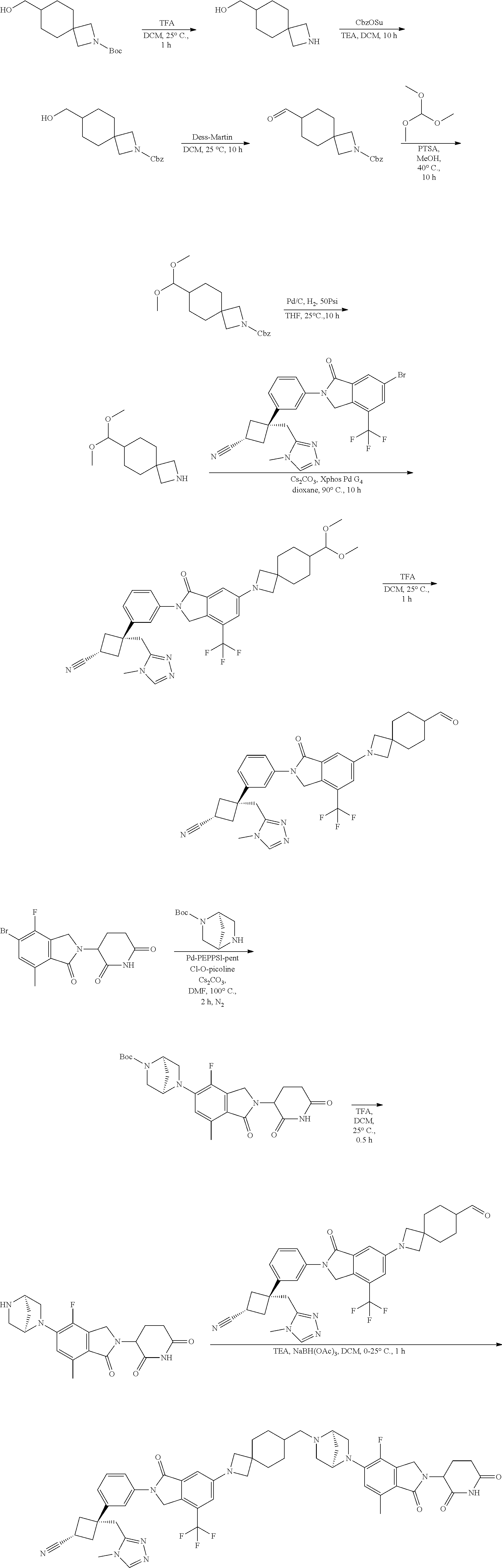

### Step 1: Preparation of 2-azaspiro[3.5]nonan-7-ylmethanol

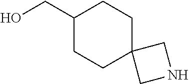 [SMILES: OCC1CCC2(CC1)CNC2]

To a solution of tert-butyl 7-(hydroxymethyl)-2-azaspiro[3.5]nonane-2-carboxylate (1 g, 4 mmol) in dichloromethane (8 mL) was added trifluoroacetic acid (3 mL). The reaction was stirred at 25° C. for 1 h, then concentrated under reduced pressure to afford 2-azaspiro[3.5]nonan-7-ylmethanol trifluoroacetate (1.00 g, 95%) as a colorless oil, which was used directly in the next step.

### Step 2: Preparation of benzyl 7-(hydroxymethyl)-2-azaspiro[3.5]nonane-2-carboxylate

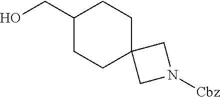 [SMILES: O=C(OCc1ccccc1)N1CC2(CCC(CO)CC2)C1]

To a solution of 2-azaspiro[3.5]nonan-7-ylmethanol trifluoroacetate (1.0 g, 4 mmol) in dichloromethane (10 mL) was added triethylamine (1.13 g, 11 mmol) and benzyl (2,5-dioxopyrrolidin-1-yl) carbonate (1.39 g, 5.6 mmol). The mixture was stirred at 25° C. for 10 h, then concentrated under reduced pressure. The residue was purified by prep-HPLC (32%-62% acetonitrile in water (0.2% formic acid) over 11 min) to afford benzyl 7-(hydroxymethyl)-2-azaspiro[3.5]nonane-2-carboxylate (700 mg, 65%) as a colorless oil. MS (ESI) m/z: 290.1 [M+H]⁺.

### Step 3: Preparation of benzyl 7-formyl-2-azaspiro[3.5]nonane-2-carboxylate

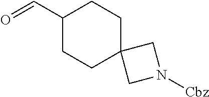 [SMILES: O=CC1CCC2(CC1)CN(C(=O)OCc1ccccc1)C2]

To a solution of benzyl 7-(hydroxymethyl)-2-azaspiro[3.5]nonane-2-carboxylate (1.40 g, 4.8 mmol) in dichloromethane (30 mL) was added 1,1,1-tris(acetyloxy)-1,1-dihydro-1,2-benziodoxol-3-(1H)-one (2.3 mL, 7 mmol). The mixture was stirred at 25° C. for 10 h, then diluted with water (30 mL) and extracted with dichloromethane (10 mL×3). The combined organic phase was washed with brine (50 mL), dried over anhydrous sodium sulfate, filtered, and concentrated. The residue was purified by column chromatography on silica gel (petroleum ether/ethyl acetate=30/1 to 10/1) to afford benzyl 7-formyl-2-azaspiro[3.5]nonane-2-carboxylate (700 mg, 50%) as a colorless oil. MS (ESI) m/z: 288.1 [M+H]⁺; ¹H NMR (DMSO-d₆) δ 9.56 (s, 1H), 7.39-7.31 (m, 5H), 5.03 (s, 2H), 3.67-3.50 (m, 4H), 2.34-2.18 (m, 1H), 1.85-1.72 (m, 4H), 1.55-1.45 (m, 2H), 1.37-1.27 (m, 2H).

### Step 4: Preparation of benzyl 7-(dimethoxymethyl)-2-azaspiro[3.5]nonane-2-carboxylate

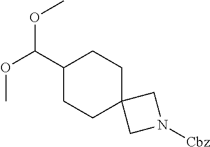 [SMILES: COC(OC)C1CCC2(CC1)CN(C(=O)OCc1ccccc1)C2]

To a solution of benzyl 7-formyl-2-azaspiro[3.5]nonane-2-carboxylate (700 mg, 2.4 mmol) and trimethoxymethane (5.17 g, 49 mmol) in methanol (10 mL) was added 4-methylbenzenesulfonic acid (126 mg, 0.7 mmol). The mixture was stirred at 40° C. for 10 h, then concentrated under reduced pressure. The residue was purified by prep-HPLC (45%-75% acetonitrile in water (ammonium hydroxide) over 9 min) to afford benzyl 7-(dimethoxymethyl)-2-azaspiro[3.5]nonane-2-carboxylate (300 mg, 37%) as a colorless oil. MS (ESI) m/z: 334.2 [M+H]⁺.

### Step 5: Preparation of 7-(dimethoxymethyl)-2-azaspiro[3.5]nonane

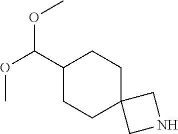

To a solution of benzyl 7-(dimethoxymethyl)-2-azaspiro[3.5]nonane-2-carboxylate (300 mg, 0.9 mmol) in tetrahydrofuran (10 mL) was added 10% palladium on carbon (100 mg) under nitrogen atmosphere. The suspension was degassed and purged with hydrogen for three times. The mixture was stirred under hydrogen (50 psi) at 25° C. for 10 h, then filtered and the filtrate was concentrated under reduced pressure to afford 7-(dimethoxymethyl)-2-azaspiro[3.5]nonane (179 mg, 99%) as a white solid. ¹H NMR (400 MHz, CDCl₃) δ 3.92-3.84 (m, 2H), 3.44-3.38 (m, 2H), 3.35 (s, 2H), 3.26 (s, 6H), 2.00 (d, J=13.2 Hz, 2H), 1.74-1.56 (m, 2H), 1.54-1.39 (m, 1H), 1.29 (t, J=2.8, 13.2 Hz, 2H), 1.01-0.82 (m, 2H).

### Step 6: Preparation of 3-[3-[6-[7-(dimethoxymethyl)-2-azaspiro[3.5]nonan-2-yl]-1-oxo-4-(trifluoromethyl)isoindolin-2-yl]phenyl]-3-[(4-methyl-1,2,4-triazol-3-yl)methyl]cyclobutanecarbonitrile

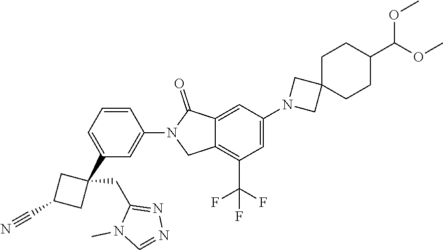 [SMILES: COC(OC)C1CCC2(CC1)CN(c1cc3c(c(C(F)(F)F)c1)CN(c1cccc([C@]4(Cc5nncn5C)C[C@@H](C#N)C4)c1)C3=O)C2]

A mixture of 7-(dimethoxymethyl)-2-azaspiro[3.5]nonane (150 mg, 0.8 mmol), 3-[3-[6-bromo-1-oxo-4-(trifluoromethyl)isoindolin-2-yl]phenyl]-3-[(4-methyl-1,2,4-triazol-3-yl)methyl]cyclobutanecarbonitrile (439 mg, 0.8 mmol), cesium carbonate (736 mg, 2 mmol), chloro[(4,5-bis(diphenylphosphino)-9,9-dimethylxanthene)-2-(2′-amino-1,1′-biphenyl)]palladium(II) (194 mg, 0.2 mmol) in 1,4-dioxane (5 mL) was degassed and purged with nitrogen for three times, then the mixture was stirred at 90° C. for 10 h under nitrogen atmosphere. The reaction was diluted with water (30 mL) and extracted with ethyl acetate (10 mL×3). The combined organic phase was washed with brine (30 mL), dried over anhydrous sodium sulfate, filtered, and concentrated. The residue was purified by column chromatography on silica gel (petroleum ether/ethyl acetate=1/1 then dichloromethane:methanol=30:1) to afford 3-[3-[6-[7-(dimethoxymethyl)-2-azaspiro[3.5]nonan-2-yl]-1-oxo-4-(trifluoromethyl)isoindolin-2-yl]phenyl]-3-[(4-methyl-1,2,4-triazol-3-yl)methyl]cyclobutanecarbonitrile (420 mg, 86%) as a yellow solid. MS (ESI) m/z: 649.4 [M+H]⁺.

### Step 7: Preparation of 3-[3-[6-(7-formyl-2-azaspiro[3.5]nonan-2-yl)-1-oxo-4-(trifluoromethyl)isoindolin-2-yl]phenyl]-3-[(4-methyl-1,2,4-triazol-3-yl)methyl]cyclobutanecarbonitrile

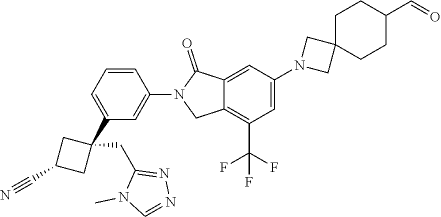 [SMILES: Cn1cnnc1C[C@]1(c2cccc(N3Cc4c(cc(N5CC6(CCC(C=O)CC6)C5)cc4C(F)(F)F)C3=O)c2)C[C@H](C#N)C1]

To a solution of 3-[3-[6-[7-(dimethoxymethyl)-2-azaspiro[3.5]nonan-2-yl]-1-oxo-4-(trifluoromethyl)isoindolin-2-yl]phenyl]-3-[(4-methyl-1,2,4-triazol-3-yl)methyl]cyclobutanecarbonitrile (150 mg, 0.2 mmol) in dichloromethane (1 mL) was added trifluoroacetic acid (461 mg, 4 mmol). The mixture was stirred at 25° C. for 1 h, then concentrated under reduced pressure to afford 3-[3-[6-(7-formyl-2-azaspiro[3.5]nonan-2-yl)-1-oxo-4-(trifluoromethyl)isoindolin-2-yl]phenyl]-3-[(4-methyl-1,2,4-triazol-3-yl)methyl]cyclobutanecarbonitrile (139 mg, 99%) as a yellow oil. MS (ESI) m/z: 603.4 [M+H]⁺.

### Step 8: Preparation of tert-butyl (1R,4R)-5-[2-(2,6-dioxo-3-piperidyl)-4-fluoro-7-methyl-1-oxo-isoindolin-5-yl]-2,5-diazabicyclo[2.2.1]heptane-2-carboxylate

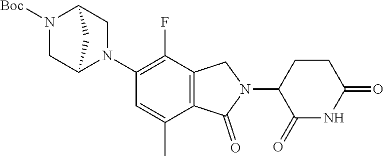 [SMILES: Cc1cc(N2C[C@H]3C[C@@H]2CN3C(=O)OC(C)(C)C)c(F)c2c1C(=O)N(C1CCC(=O)NC1=O)C2]

To a solution of 3-(5-bromo-4-fluoro-7-methyl-1-oxo-isoindolin-2-yl)piperidine-2,6-dione (300 mg, 0.8 mmol), tert-butyl (1R,4R)-2,5-diazabicyclo[2.2.1]heptane-2-carboxylate (251 mg, 1 mmol), dichloro[1,3-bis(2,6-di-3-pentylphenyl)imidazol-2-ylidene](3-chloropyridyl)palladium(II) (82 mg, 0.08 mmol) and cesium carbonate (826 mg, 2.5 mmol) in N, N-dimethylformamide (5 mL) was degassed and purged with nitrogen for three times, then the mixture was stirred at 100° C. for 2 h under nitrogen. The reaction was diluted with water (30 mL) and extracted with ethyl acetate (30 mL×3). The combined organic layers were concentrated under reduced pressure. The residue was purified by silica gel chromatography (dichloromethane:methanol=50/1 to 10/1) to afford tert-butyl (1R,4R)-5-[2-(2,6-dioxo-3-piperidyl)-4-fluoro-7-methyl-1-oxo-isoindolin-5-yl]-2,5-diazabicyclo[2.2.1]heptane-2-carboxylate (285 mg, 71%) as a white solid. MS (ESI) m/z: 473.3 [M+H]⁺.

### Step 9: Preparation of 3-[5-[(1R,4R)-2,5-diazabicyclo[2.2.1]heptan-2-yl]-4-fluoro-7-methyl-1-oxo-isoindolin-2-yl]piperidine-2,6-dione

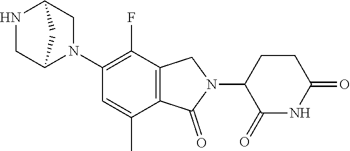 [SMILES: Cc1cc(N2C[C@H]3C[C@@H]2CN3)c(F)c2c1C(=O)N(C1CCC(=O)NC1=O)C2]

To a solution of tert-butyl (1R,4R)-5-[2-(2,6-dioxo-3-piperidyl)-4-fluoro-7-methyl-1-oxo-isoindolin-5-yl]-2,5-diazabicyclo[2.2.1]heptane-2-carboxylate (285 mg, 0.6 mmol) in dichloromethane (2 mL) was added trifluoroacetic acid (768 mg, 6.7 mmol). The mixture was stirred at 25° C. for 0.5 h, then concentrated under reduced pressure. The residue was purified by prep-TLC (1%-30% acetonitrile in water (formic acid) over 10 min) to afford 3-[5-[(1R,4R)-2,5-diazabicyclo[2.2.1]heptan-2-yl]-4-fluoro-7-methyl-1-oxo-isoindolin-2-yl]piperidine-2,6-dione (202 mg, 89%) as a white solid. MS (ESI) m/z: 373.1 [M+H]⁺.

### Step 10: Preparation of 3-[3-[6-[7-[[(1R,4R)-5-[2-(2,6-dioxo-3-piperidyl)-4-fluoro-7-methyl-1-oxo-isoindolin-5-yl]-2,5-diazabicyclo[2.2.1]heptan-2-yl]methyl]-2-azaspiro[3.5]nonan-2-yl]-1-oxo-4-(trifluoromethyl)isoindolin-2-yl]phenyl]-3-[(4-methyl-1,2,4-triazol-3-yl)methyl]cyclobutanecarbonitrile

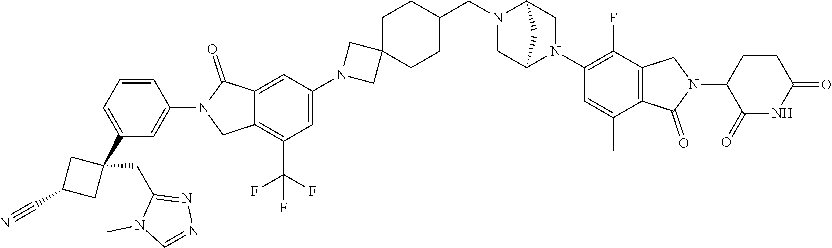 [SMILES: Cc1cc(N2C[C@H]3C[C@@H]2CN3CC2CCC3(CC2)CN(c2cc4c(c(C(F)(F)F)c2)CN(c2cccc([C@]5(Cc6nncn6C)C[C@@H](C#N)C5)c2)C4=O)C3)c(F)c2c1C(=O)N(C1CCC(=O)NC1=O)C2]

To a solution of 3-[5-[(1R,4R)-2,5-diazabicyclo[2.2.1]heptan-2-yl]-4-fluoro-7-methyl-1-oxo-isoindolin-2-yl]piperidine-2,6-dione (93 mg, 0.3 mmol) and 3-[3-[6-(7-formyl-2-azaspiro[3.5]nonan-2-yl)-1-oxo-4-(trifluoromethyl)isoindolin-2-yl]phenyl]-3-[(4-methyl-1,2,4-triazol-3-yl)methyl]cyclobutanecarbonitrile (150 mg, 0.3 mmol) in dichloromethane (1 mL) was added trimethylamine (76 mg, 0.8 mmol). The mixture was stirred at 0° C. for 0.5 h, followed by the addition of sodium triacetoxyborohydride (106 mg, 0.5 mmol). The resulting mixture was stirred at 25° C. for 0.5 h, then concentrated under reduced pressure. The residue was purified by prep-TLC (20%-50% acetonitrile in water (formic acid) over 10 min) to afford 3-[3-[6-[7-[[(1R,4R)-5-[2-(2,6-dioxo-3-piperidyl)-4-fluoro-7-methyl-1-oxo-isoindolin-5-yl]-2,5-diazabicyclo[2.2.1]heptan-2-yl]methyl]-2-azaspiro[3.5]nonan-2-yl]-1-oxo-4-(trifluoromethyl)isoindolin-2-yl]phenyl]-3-[(4-methyl-1,2,4-triazol-3-yl)methyl]cyclobutanecarbonitrile formate (78.9 mg, 28%) as an off-white solid. MS (ESI) m/z: 824.6 [M+H]⁺; ¹H NMR (400 MHz, DMSO-d₆) δ 10.97 (s, 1H), 8.17 (s, 1H), 8.15 (s, 1H), 7.87-7.82 (m, 1H), 7.42 (s, 1H), 7.33 (t, J=8.0 Hz, 1H), 6.91 (d, J=3.2 Hz, 2H), 6.81 (d, J=7.6 Hz, 1H), 6.60 (d, J=7.6 Hz, 1H), 5.01 (dd, J=5.2, 13.2 Hz, 1H), 4.94 (s, 2H), 4.46 (s, 1H), 4.36 (d, J=16.8 Hz, 1H), 4.20 (d, J=17.2 Hz, 1H), 3.66 (s, 2H), 3.61 (s, 2H), 3.59 (s, 2H), 3.30 (s, 3H), 3.26-3.19 (m, 2H), 2.94 (dd, J=9.2, 14.8 Hz, 2H), 2.87 (d, J=8.0 Hz, 4H), 2.78 (s, 3H), 2.73-2.59 (m, 2H), 2.57 (s, 1H), 2.48-2.32 (m, 4H), 1.97 (s, 4H), 1.82 (d, J=8.4 Hz, 1H), 1.78-1.68 (m, 2H), 1.51-1.41 (m, 2H), 1.39-1.30 (m, 1H), 1.00-0.88 (m, 2H).

### Example 151: Exemplary synthesis of 3-[7-methyl-2-[4-[4-[[4-[[6-[3-[3-[(4-methyl-1,2,4-triazol-3-yl)methyl]oxetan-3-yl]phenyl]-7-oxo-5H-pyrrolo [3,4-b]pyridin-2-yl]methyl]piperazin-1-yl]methyl]cyclohexoxy]-1-piperidyl]-5-oxo-7H-pyrrolo [3,4-b]pyridin-6-yl]piperidine-2,6-dione

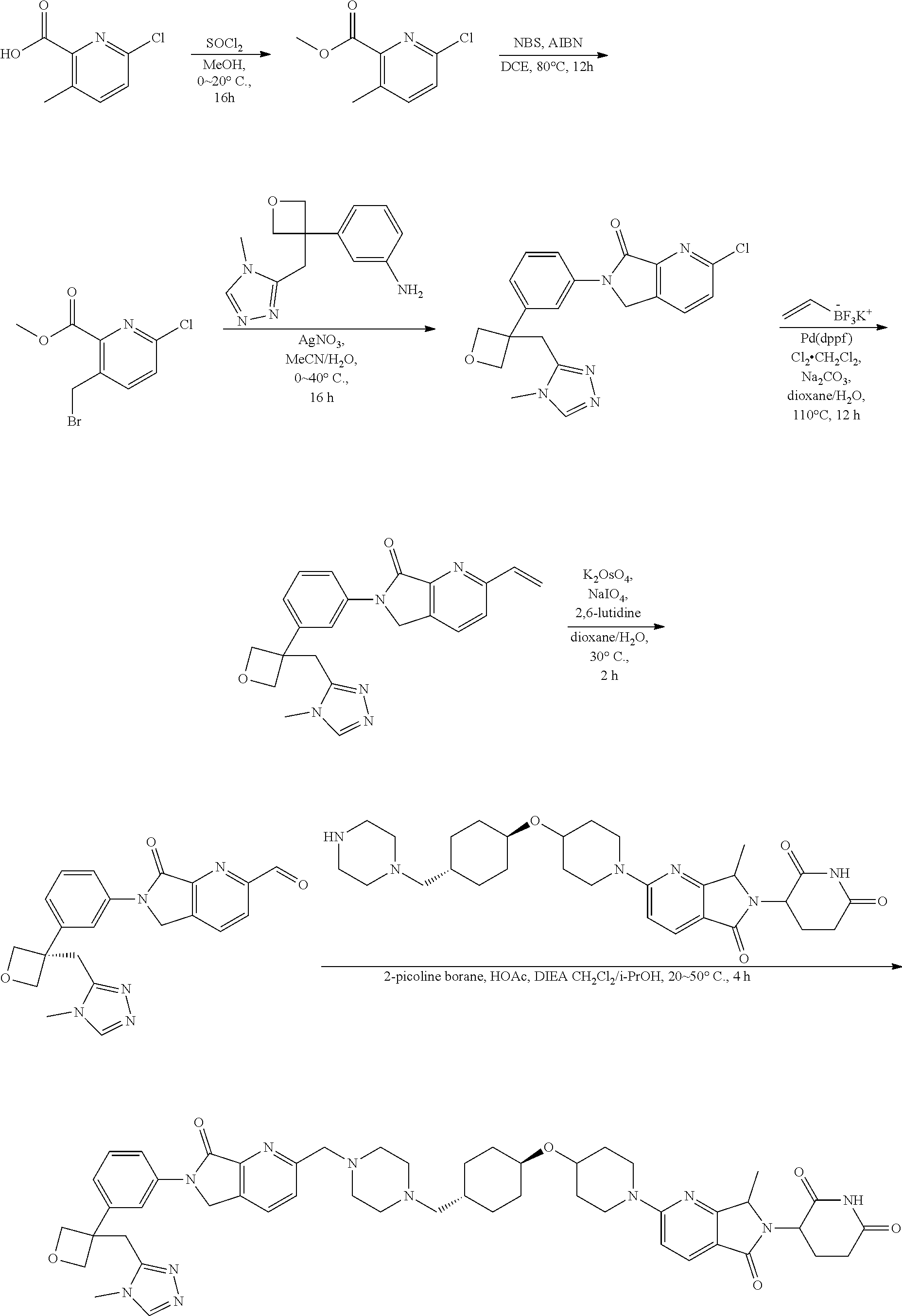

### Step 1: Preparation of methyl 6-chloro-3-methyl-pyridine-2-carboxylate

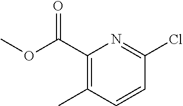

To a solution of 6-chloro-3-methyl-pyridine-2-carboxylic acid (10 g, 58 mmol) in methanol (100 mL) was dropwise added thionyl chloride (12.7 mL, 175 mmol) at 0° C. The mixture was stirred at 20° C. for 16 h, then concentrated. The residue was diluted with saturated sodium carbonate solution (100 mL) and extracted with ethyl acetate (100 mL×3). The combined organic layer was washed with brine (20 mL), dried over anhydrous sodium sulfate, filtered, and concentrated to afford methyl 6-chloro-3-methyl-pyridine-2-carboxylate (11.2 g, crude) as yellow solid. MS (ESI) m/z: 186.0 [M+H]⁺.

### Step 2: Preparation of methyl 3-(bromomethyl)-6-chloro-pyridine-2-carboxylate

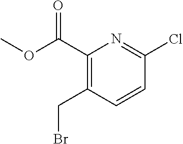

To a solution of methyl 6-chloro-3-methyl-pyridine-2-carboxylate (1.0 g, 5 mmol) and N-bromosuccinimide (1.42 g, 8 mmol) in 1,2-dichloroethane (15 mL) was added 2,2′-azobis(2-methylpropionitrile) (175 mg, 1 mmol). The mixture was stirred at 80° C. for 12 h under nitrogen atmosphere, then concentrated. The residue was purified by flash column chromatography (0-3% ethyl acetate in petroleum ether) to afford methyl 3-(bromomethyl)-6-chloro-pyridine-2-carboxylate (1.01 g, 55%) as white solid. MS (ESI) m/z: 265.9 [M+H]⁺.

### Step 3: Preparation of 2-chloro-6-[3-[3-[(4-methyl-1,2,4-triazol-3-yl)methyl]oxetan-3-yl]phenyl]-5H-pyrrolo[3,4-b]pyridin-7-one

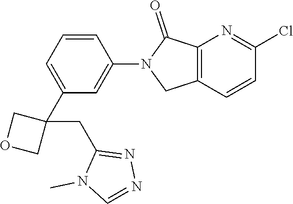 [SMILES: Cn1cnnc1CC1(c2cccc(N3Cc4ccc(Cl)nc4C3=O)c2)COC1]

To a solution of 3-[3-[(4-methyl-1,2,4-triazol-3-yl)methyl]oxetan-3-yl]aniline trifluoroacetate (523 mg, 1.5 mmol) and methyl 3-(bromomethyl)-6-chloro-pyridine-2-carboxylate (500 mg, 1.5 mmol) in acetonitrile (20 mL) was added silver nitrate (310 mg, 1.8 mmol) in water (10 mL) at 0° C. The mixture was stirred at 40° C. for 16 h, then saturated sodium carbonate solution was added to adjust the pH to 8-9, the mixture was extracted with dichloromethane (30 mL×3). The combined organic layer was washed with brine (20 mL), dried over anhydrous sodium sulfate, filtered, and concentrated. The residue was purified by flash column chromatography (0-6% methanol in dichloromethane) to afford 2-chloro-6-[3-[3-[(4-methyl-1,2,4-triazol-3-yl)methyl]oxetan-3-yl]phenyl]-5H-pyrrolo[3,4-b]pyridin-7-one (367 mg, 64%) as white solid. MS (ESI) m/z: 396.2 [M+H]⁺.

### Step 4: Preparation of 6-[3-[3-[(4-methyl-1,2,4-triazol-3-yl)methyl]oxetan-3-yl]phenyl]-2-vinyl-5H-pyrrolo[3,4-b]pyridin-7-one

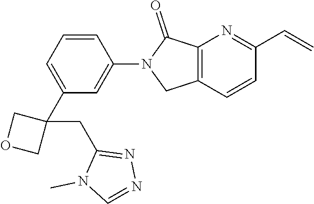 [SMILES: C=Cc1ccc2c(n1)C(=O)N(c1cccc(C3(Cc4nncn4C)COC3)c1)C2]

To a mixture of 2-chloro-6-[3-[3-[(4-methyl-1,2,4-triazol-3-yl)methyl]oxetan-3-yl]phenyl]-5H-pyrrolo[3,4-b]pyridin-7-one (367 mg, 0.9 mmol) and potassium vinyltrifluoroborate (373 mg, 2.8 mmol) in 1,4-dioxane (10 mL) and water (1.0 mL) was added sodium carbonate (246 mg, 2.3 mmol) and [1,1′-bis(diphenylphosphino)ferrocene]dichloropalladium(II) dichloromethane (75.7 mg, 0.09 mmol). The mixture was stirred at 110° C. for 12 h under nitrogen atmosphere. The reaction crude was dried over anhydrous sodium sulfate, filtered, and concentrated. The residue was purified by flash column chromatography (2-6% methanol in dichloromethane) to afford 6-[3-[3-[(4-methyl-1,2,4-triazol-3-yl)methyl]oxetan-3-yl]phenyl]-2-vinyl-5H-pyrrolo[3,4-b]pyridin-7-one (330 mg, 90%) as brown solid. MS (ESI) m/z: 388.1 [M+H]⁺.

### Step 5: Preparation of 6-[3-[3-[(4-methyl-1,2,4-triazol-3-yl)methyl]oxetan-3-yl]phenyl]-7-oxo-5H-pyrrolo[3,4-b]pyridine-2-carbaldehyde

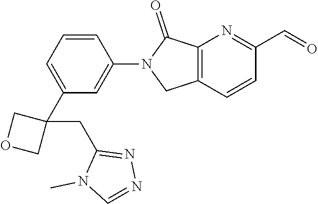 [SMILES: Cn1cnnc1CC1(c2cccc(N3Cc4ccc(C=O)nc4C3=O)c2)COC1]

To a solution of 6-[3-[3-[(4-methyl-1,2,4-triazol-3-yl)methyl]oxetan-3-yl]phenyl]-2-vinyl-5H-pyrrolo[3,4-b]pyridin-7-one (330 mg, 0.8 mmol) in water (5.0 mL) and 1,4-dioxane (15 mL) was added 2,6-dimethylpyridine (194 μL, 1.7 mmol), potassium osmate(VI) dihydrate (6.1 mg, 17 μmol) and sodium periodate (713 mg, 3 mmol). The mixture was stirred at 30° C. for 2 h, then diluted with water (20 mL) and extracted with dichloromethane/methanol (10/1, 30 mL×3). The combined organic layer was washed with brine (50 mL), dried over anhydrous sodium sulfate, filtered, and concentrated. The residue was purified by flash column chromatography (2-8% methanol in dichloromethane) to afford 6-[3-[3-[(4-methyl-1,2,4-triazol-3-yl)methyl]oxetan-3-yl]phenyl]-7-oxo-5H-pyrrolo[3,4-b]pyridine-2-carbaldehyde (189 mg, 56%) as white solid. MS (ESI) m/z: 390.2 [M+H]⁺.

### Step 6: Preparation of 3-[7-methyl-2-[4-[4-[[4-[[6-[3-[3-[(4-methyl-1,2,4-triazol-3-yl)methyl]oxetan-3-yl]phenyl]-7-oxo-5H-pyrrolo[3,4-b]pyridin-2-yl]methyl]piperazin-1-yl]methyl]cyclohexoxy]-1-piperidyl]-5-oxo-7H-pyrrolo[3,4-b]pyridin-6-yl]piperidine-2,6-dione

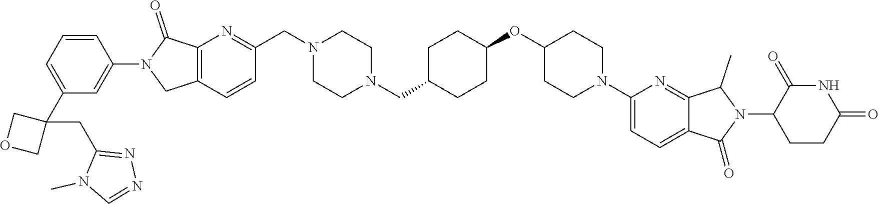 [SMILES: CC1c2nc(N3CCC(O[C@H]4CC[C@H](CN5CCN(Cc6ccc7c(n6)C(=O)N(c6cccc(C8(Cc9nncn9C)COC8)c6)C7)CC5)CC4)CC3)ccc2C(=O)N1C1CCC(=O)NC1=O]

To a mixture of 3-[7-methyl-5-oxo-2-[4-[4-(piperazin-1-ylmethyl)cyclohexoxy]-1-piperidyl]-7H-pyrrolo[3,4-b]pyridin-6-yl]piperidine-2,6-dione trifluoroacetate (226 mg, 0.2 mmol, freed by N, N-diisopropylethylamine (240 μL, 1.4 mmol)), 6-[3-[3-[(4-methyl-1,2,4-triazol-3-yl)methyl]oxetan-3-yl]phenyl]-7-oxo-5H-pyrrolo[3,4-b]pyridine-2-carbaldehyde (80 mg, 0.2 mmol) in 1,2-dichloroethane (5.0 mL) was added acetic acid (140 μL). The mixture was stirred at 50° C. for 2 h, then cooled to 20° C. and 2-methylpyridine borane (46 mg, 0.4 mmol) was added. The mixture was stirred at 20° C. for 2 h, then concentrated. The residue was purified by prep-HPLC (0-34% acetonitrile in water (0.2% formic acid) over 25 min). The crude product was further purified by prep-HPLC (15-50% acetonitrile in water (ammonium bicarbonate) over 32 min) to afford 3-[7-methyl-2-[4-[4-[[4-[[6-[3-[3-[(4-methyl-1,2,4-triazol-3-yl)methyl]oxetan-3-yl]phenyl]-7-oxo-5H-pyrrolo[3,4-b]pyridin-2-yl]methyl]piperazin-1-yl]methyl]cyclohexoxy]-1-piperidyl]-5-oxo-7H-pyrrolo[3,4-b]pyridin-6-yl]piperidine-2,6-dione (53.7 mg, 30%) as white solid. MS (ESI) m/z: 912.4 [M+H]⁺; ¹H NMR (400 MHz, DMSO-d₆) δ 10.88 (d, J=12.8 Hz, 1H), 8.19 (s, 1H), 8.10 (br d, J=8.0 Hz, 1H), 7.94 (dd, J=1.6, 8.0 Hz, 1H), 7.72-7.64 (m, 2H), 7.37 (t, J=8.0 Hz, 1H), 7.32 (s, 1H), 6.88 (dd, J=3.6, 8.8 Hz, 1H), 6.80 (br d, J=7.2 Hz, 1H), 4.96 (d, J=6.0 Hz, 2H), 4.93-4.86 (m, 4H), 4.69 (br dd, J=5.2, 12.4 Hz, 1H), 4.51-4.38 (m, 1H), 4.16-4.01 (m, 2H), 3.71 (br d, J=3.6 Hz, 3H), 3.51 (s, 2H), 3.32-3.24 (m, 4H), 2.92 (s, 3H), 2.84-2.73 (m, 1H), 2.59 (br dd, J=2.8, 7.6 Hz, 2H), 2.52 (br d, J=2.0 Hz, 2H), 2.47-2.40 (m, 3H), 2.40-2.25 (m, 3H), 2.07 (br d, J=9.2 Hz, 1H), 2.03-1.96 (m, 1H), 1.92 (br d, J=10.0 Hz, 2H), 1.88-1.80 (m, 2H), 1.76 (br d, J=11.2 Hz, 2H), 1.38 (br dd, J=6.8, 14.4 Hz, 6H), 1.21-1.07 (m, 2H), 0.98-0.78 (m, 2H).

### Example 152: Exemplary synthesis of 3-[3-[6-[[4-[[4-[[1-[6-(2,6-dioxo-3-piperidyl)-7-methyl-5-oxo-7H-pyrrolo[3,4-b]pyridin-2-yl]-4-piperidyl]oxy]cyclohexyl]methyl]-2,2-dimethyl-piperazin-1-yl]methyl]-1-oxo-4-(trifluoromethyl)isoindolin-2-yl]phenyl]-3-[(4-methyl-1,2,4-triazol-3-yl)methyl]cyclobutanecarbonitrile

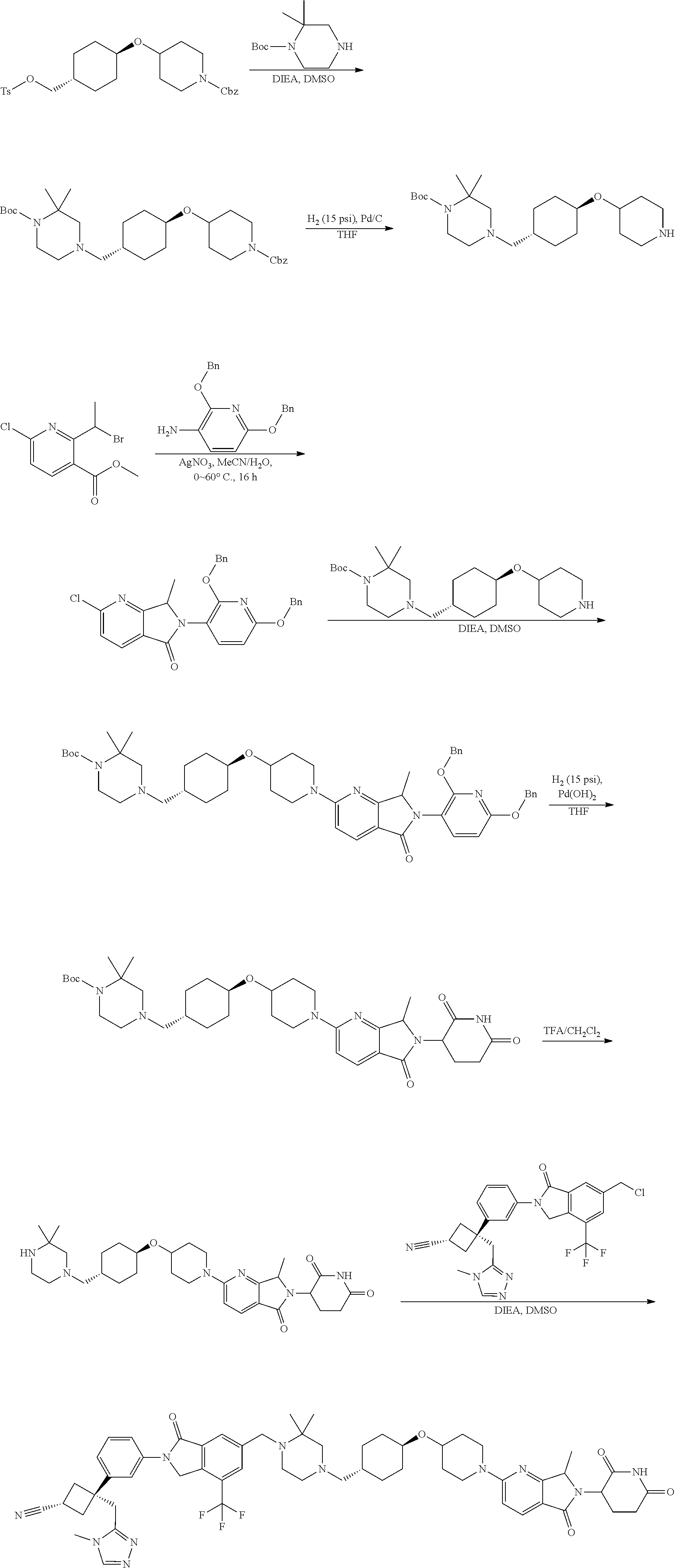

### Step 1: Preparation of tert-butyl 4-[[4-[(1-benzyloxycarbonyl-4-piperidyl)oxy]cyclohexyl]methyl]-2,2-dimethyl-piperazine-1-carboxylate

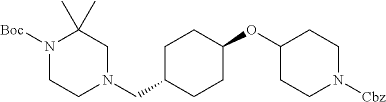 [SMILES: CC(C)(C)OC(=O)N1CCN(C[C@H]2CC[C@H](OC3CCN(C(=O)OCc4ccccc4)CC3)CC2)CC1(C)C]

To a solution of tert-butyl 2,2-dimethylpiperazine-1-carboxylate (6.15 g, 29 mmol) and N, N-diisopropylethylamine (8.33 mL, 48 mmol) in dimethyl sulfoxide (90 mL) was added benzyl 4-[4-(p-tolylsulfonyloxymethyl)cyclohexoxy]piperidine-1-carboxylate (8.0 g, 16 mmol) under nitrogen atmosphere. The mixture was stirred at 120° C. for 12 h. To the reaction mixture was added water (100 mL), and the aqueous phase was extracted with ethyl acetate (100 mL×3). The combined organic phase was washed with brine (50 mL), dried over anhydrous sodium sulfate, filtered, and concentrated. The residue was purified by flash column chromatography (0-12% tetrahydrofuran in petroleum ether) to afford tert-butyl 4-[[4-[(1-benzyloxycarbonyl-4-piperidyl)oxy]cyclohexyl]methyl]-2,2-dimethyl-piperazine-1-carboxylate (7.0 g, 81%) as yellow solid. MS (ESI) m/z: 544.5 [M+H]⁺; ¹H NMR (400 MHz, CDCl₃) δ 7.40-7.27 (m, 5H), 5.13 (s, 2H), 3.93-3.80 (m, 2H), 3.66-3.55 (m, 1H), 3.44-3.38 (m, 2H), 3.34-3.24 (m, 1H), 3.17 (ddd, J=3.2, 9.6, 13.2 Hz, 2H), 2.33 (t, J=5.2 Hz, 2H), 2.09 (s, 2H), 2.04 (d, J=7.2 Hz, 2H), 1.97 (br d, J=10.4 Hz, 2H), 1.91-1.76 (m, 4H), 1.56-1.49 (m, 2H), 1.46 (s, 9H), 1.44-1.40 (m, 1H), 1.37 (s, 6H), 1.28-1.20 (m, 2H), 0.95-0.81 (m, 2H).

### Step 2: Preparation of tert-butyl 2,2-dimethyl-4-[[4-(4-piperidyloxy)cyclohexyl]methyl]piperazine-1-carboxylate

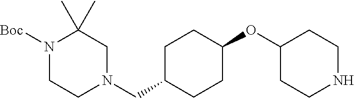 [SMILES: CC(C)(C)OC(=O)N1CCN(C[C@H]2CC[C@H](OC3CCNCC3)CC2)CC1(C)C]

To a solution of tert-butyl 4-[[4-[(1-benzyloxycarbonyl-4-piperidyl)oxy]cyclohexyl]methyl]-2,2-dimethyl-piperazine-1-carboxylate (7.0 g, 13 mmol) in tetrahydrofuran (100 mL) was added 10% palladium over carbon (1.75 g) under nitrogen atmosphere. The suspension was degassed and purged with hydrogen for three times. The mixture was stirred under hydrogen (15 psi) at 25° C. for 4 h, then filtered, and concentrated to afford tert-butyl 2,2-dimethyl-4-[[4-(4-piperidyloxy)cyclohexyl]methyl]piperazine-1-carboxylate (4.54 g, 86%) as brown solid. ¹H NMR (400 MHz, CDCl₃) δ 3.75 (t, J=6.4 Hz, 1H), 3.50-3.38 (m, 3H), 3.30 (br t, J=4.0 Hz, 1H), 3.09 (td, J=3.6, 12.8 Hz, 2H), 2.64-2.55 (m, 2H), 2.33 (t, J=5.2 Hz, 2H), 2.09 (s, 2H), 2.04 (d, J=7.6 Hz, 2H), 1.98 (br d, J=10.0 Hz, 2H), 1.89-1.82 (m, 5H), 1.46 (s, 9H), 1.37 (s, 6H), 1.29-1.18 (m, 3H), 0.88 (br d, J=14.0 Hz, 2H).

### Step 3: Preparation of 2-chloro-6-(2,6-dibenzyloxy-3-pyridyl)-7-methyl-7H-pyrrolo[3,4-b]pyridin-5-one

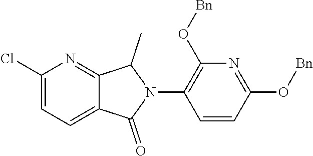 [SMILES: CC1c2nc(Cl)ccc2C(=O)N1c1ccc(OCc2ccccc2)nc1OCc1ccccc1]

To a solution of methyl 2-(1-bromoethyl)-6-chloro-pyridine-3-carboxylate (8.0 g, 29 mmol) and 2,6-dibenzyloxypyridin-3-amine (10.5 g, 34 mmol) in acetonitrile (240 mL) was added the solution of silver nitrate (6.34 g, 37 mmol) in water (120 mL) at 0° C. The mixture was stirred at 60° C. for 16 h, then saturated sodium carbonate solution was added to adjust the pH to 8-9 and the mixture was extracted with ethyl acetate (200 mL×3). The combined organic layer was washed with brine (200 mL), dried over sodium sulfate, filtered, and concentrated. The residue was purified by flash column chromatography (10-20% ethyl acetate in petroleum ether) to afford 2-chloro-6-(2,6-dibenzyloxy-3-pyridyl)-7-methyl-7H-pyrrolo[3,4-b]pyridin-5-one (6.49 g, 46%) as red gum. MS (ESI) m/z: 472.1 [M+H]⁺.

### Step 4: Preparation of tert-butyl 4-[[4-[[1-[6-(2,6-dibenzyloxy-3-pyridyl)-7-methyl-5-oxo-7H-pyrrolo[3,4-b]pyridin-2-yl]-4-piperidyl]oxy]cyclohexyl]methyl]-2,2-dimethylpiperazine-1-carboxylate

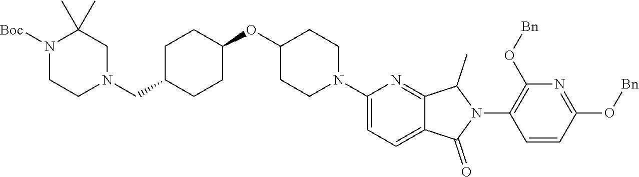 [SMILES: CC1c2nc(N3CCC(O[C@H]4CC[C@H](CN5CCN(C(=O)OC(C)(C)C)C(C)(C)C5)CC4)CC3)ccc2C(=O)N1c1ccc(OCc2ccccc2)nc1OCc1ccccc1]

To a solution of tert-butyl 2,2-dimethyl-4-[[4-(4-piperidyloxy)cyclohexyl]methyl]piperazine-1-carboxylate (1.0 g, 2 mmol) and 2-chloro-6-(2,6-dibenzyloxy-3-pyridyl)-7-methyl-7H-pyrrolo[3,4-b]pyridin-5-one (886 mg, 2 mmol) in dimethyl sulfoxide (10 mL) was added N, N-diisopropylethylamine(10 mL) at 25° C. The mixture was stirred at 140° C. for 16 h, then diluted with water (20 mL) and extracted with ethyl acetate (50 mL). The organic phase was washed with water (50 mL×3), dried over anhydrous sodium sulfate, filtered, and concentrated. The residue was purified by flash column chromatography (0-30% ethyl acetate in petroleum ether) to afford tert-butyl 4-[[4-[[1-[6-(2,6-dibenzyloxy-3-pyridyl)-7-methyl-5-oxo-7H-pyrrolo[3,4-b]pyridin-2-yl]-4-piperidyl]oxy]cyclohexyl]methyl]-2,2-dimethyl-piperazine-1-carboxylate (1.34 g, 79%) as yellow oil. MS (ESI) m/z: 423.3 [M/2+H]⁺.

### Step 5: Preparation of tert-butyl 4-[[4-[[1-[6-(2,6-dioxo-3-piperidyl)-7-methyl-5-oxo-7H-pyrrolo[3,4-b]pyridin-2-yl]-4-piperidyl]oxy]cyclohexyl]methyl]-2,2-dimethylpiperazine-1-carboxylate

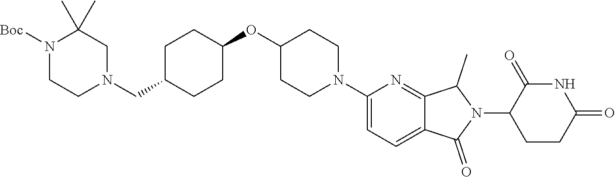 [SMILES: CC1c2nc(N3CCC(O[C@H]4CC[C@H](CN5CCN(C(=O)OC(C)(C)C)C(C)(C)C5)CC4)CC3)ccc2C(=O)N1C1CCC(=O)NC1=O]

To a solution of tert-butyl 4-[[4-[[1-[6-(2,6-dibenzyloxy-3-pyridyl)-7-methyl-5-oxo-7H-pyrrolo[3,4-b]pyridin-2-yl]-4-piperidyl]oxy]cyclohexyl]methyl]-2,2-dimethyl-piperazine-1-carboxylate (1.34 g, 1.5 mmol) in tetrahydrofuran (30 mL) was added 20% palladium hydroxide (1.3 g). The mixture was degassed and purged with hydrogen three times, then stirred under hydrogen (15 psi) at 25° C. for 16 h. The mixture was filtered through Celite pad, washed with tetrahydrofuran (20 mL×3), and concentrated. The residue was purified by flash column chromatography (0-4% methanol in dichloromethane) to afford tert-butyl 4-[[4-[[1-[6-(2,6-dioxo-3-piperidyl)-7-methyl-5-oxo-7H-pyrrolo[3,4-b]pyridin-2-yl]-4-piperidyl]oxy]cyclohexyl]methyl]-2,2-dimethylpiperazine-1-carboxylate (845 mg, 83%) as yellow oil. MS (ESI) m/z: 667.5 [M-56+H]⁺.

### Step 6: Preparation of 3-[2-[4-[4-[(3,3-dimethylpiperazin-1-yl)methyl]cyclohexoxy]-1-piperidyl]-7-methyl-5-oxo-7H-pyrrolo[3,4-b]pyridin-6-yl]piperidine-2,6-dione

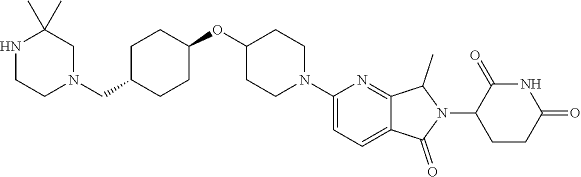 [SMILES: CC1c2nc(N3CCC(O[C@H]4CC[C@H](CN5CCNC(C)(C)C5)CC4)CC3)ccc2C(=O)N1C1CCC(=O)NC1=O]

To a mixture of tert-butyl 4-[[4-[[1-[6-(2,6-dioxo-3-piperidyl)-7-methyl-5-oxo-7H-pyrrolo[3,4-b]pyridin-2-yl]-4-piperidyl]oxy]cyclohexyl]methyl]-2,2-dimethyl-piperazine-1-carboxylate (845 mg, 1.2 mmol) in dichloromethane (9.0 mL) was added trifluoroacetic acid (3.0 mL). The mixture was stirred at 20° C. for 30 min, then concentrated. The mixture was diluted with acetonitrile (20 mL) and water (20 mL), then lyophilized to afford 3-[2-[4-[4-[(3,3-dimethylpiperazin-1-yl)methyl]cyclohexoxy]-1-piperidyl]-7-methyl-5-oxo-7H-pyrrolo[3,4-b]pyridin-6-yl]piperidine-2,6-dione trifluoroacetate (1.5 g, 95%) as yellow oil. MS (ESI) m/z: 567.2 [M+H]⁺.

### Step 7: Preparation of 3-[3-[6-[[4-[[4-[[1-[6-(2,6-dioxo-3-piperidyl)-7-methyl-5-oxo-7H-pyrrolo[3,4-b]pyridin-2-yl]-4-piperidyl]oxy]cyclohexyl]methyl]-2,2-dimethyl-piperazin-1-yl]methyl]-1-oxo-4-(trifluoromethyl)isoindolin-2-yl]phenyl]-3-[(4-methyl-1,2,4-triazol-3-yl)methyl]cyclobutanecarbonitrile

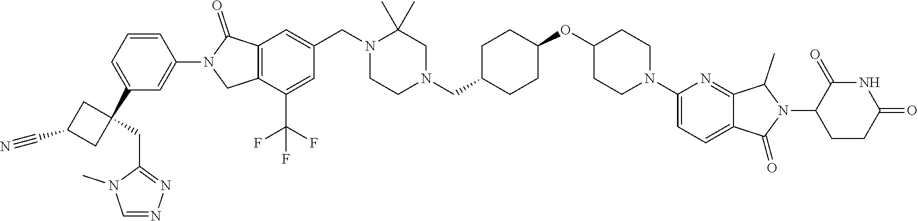 [SMILES: CC1c2nc(N3CCC(O[C@H]4CC[C@H](CN5CCN(Cc6cc7c(c(C(F)(F)F)c6)CN(c6cccc([C@]8(Cc9nncn9C)C[C@@H](C#N)C8)c6)C7=O)C(C)(C)C5)CC4)CC3)ccc2C(=O)N1C1CCC(=O)NC1=O]

To a mixture of 3-[2-[4-[4-[(3,3-dimethylpiperazin-1-yl)methyl]cyclohexoxy]-1-piperidyl]-7-methyl-5-oxo-7H-pyrrolo[3,4-b]pyridin-6-yl]piperidine-2,6-dione trifluoroacetate (120 mg, 170 μmol) and N, N-diisopropylethylamine (635 μL, 3.6 mmol) in dimethyl sulfoxide (2.0 mL) was added 3-[3-[6-(chloromethyl)-1-oxo-4-(trifluoromethyl)isoindolin-2-yl]phenyl]-3-[(4-methyl-1,2,4-triazol-3-yl)methyl]cyclobutanecarbonitrile (91 mg, 0.2 mmol). The mixture was stirred at 80° C. for 16 h. The reaction mixture was filtered and purified by prep-HPLC (28-68% acetonitrile in water (ammonium bicarbonate) over 36 min) to afford 3-[3-[6-[[4-[[4-[[1-[6-(2,6-dioxo-3-piperidyl)-7-methyl-5-oxo-7H-pyrrolo[3,4-b]pyridin-2-yl]-4-piperidyl]oxy]cyclohexyl]methyl]-2,2-dimethylpiperazin-1-yl]methyl]-1-oxo-4-(trifluoromethyl)isoindolin-2-yl]phenyl]-3-[(4-methyl-1,2,4-triazol-3-yl)methyl]cyclobutanecarbonitrile (50.5 mg, 27%) as yellow solid. MS (ESI) m/z: 1030.7 [M+H]⁺; ¹H NMR (400 MHz, DMSO-d₆) δ 10.89 (d, J=12.4 Hz, 1H), 8.17 (s, 1H), 8.01 (s, 1H), 7.92 (s, 1H), 7.87-7.82 (m, 1H), 7.70 (dd, J=3.2, 8.8 Hz, 1H), 7.47 (s, 1H), 7.35 (t, J=8.0 Hz, 1H), 6.89 (dd, J=3.2, 8.8 Hz, 1H), 6.83 (d, J=7.6 Hz, 1H), 5.09 (s, 2H), 4.70 (br dd, J=5.2, 12.8 Hz, 1H), 4.53-4.38 (m, 1H), 4.16-4.04 (m, 2H), 3.80-3.64 (m, 2H), 3.30 (br s, 5H), 3.27-3.20 (m, 2H), 2.91-2.85 (m, 4H), 2.79 (s, 3H), 2.70-2.66 (m, 1H), 2.61-2.57 (m, 1H), 2.55 (br s, 1H), 2.53 (br s, 2H), 2.37 (br s, 2H), 2.34-2.32 (m, 1H), 2.25-2.12 (m, 2H), 2.02 (br d, J=7.2 Hz, 2H), 1.98-1.90 (m, 3H), 1.89-1.83 (m, 2H), 1.82-1.76 (m, 2H), 1.39 (br dd, J=6.8, 14.4 Hz, 6H), 1.16 (br d, J=4.4 Hz, 1H), 1.12 (s, 6H), 0.94-0.83 (m, 2H).

### Example 153: Exemplary synthesis of 3-[3-[6-[[(3S)-3-[[4-[[1-[2-(2,6-dioxo-3-piperidyl)-4-fluoro-1-oxoisoindolin-5-yl]-4-piperidyl]methyl]-2,2-dimethyl-piperazin-1-yl]methyl]-1-piperidyl]methyl]-1-oxo-4-(trifluoromethyl)isoindolin-2-yl]phenyl]-3-[(4-methyl-1,2,4-triazol-3-yl)methyl]cyclobutanecarbonitrile

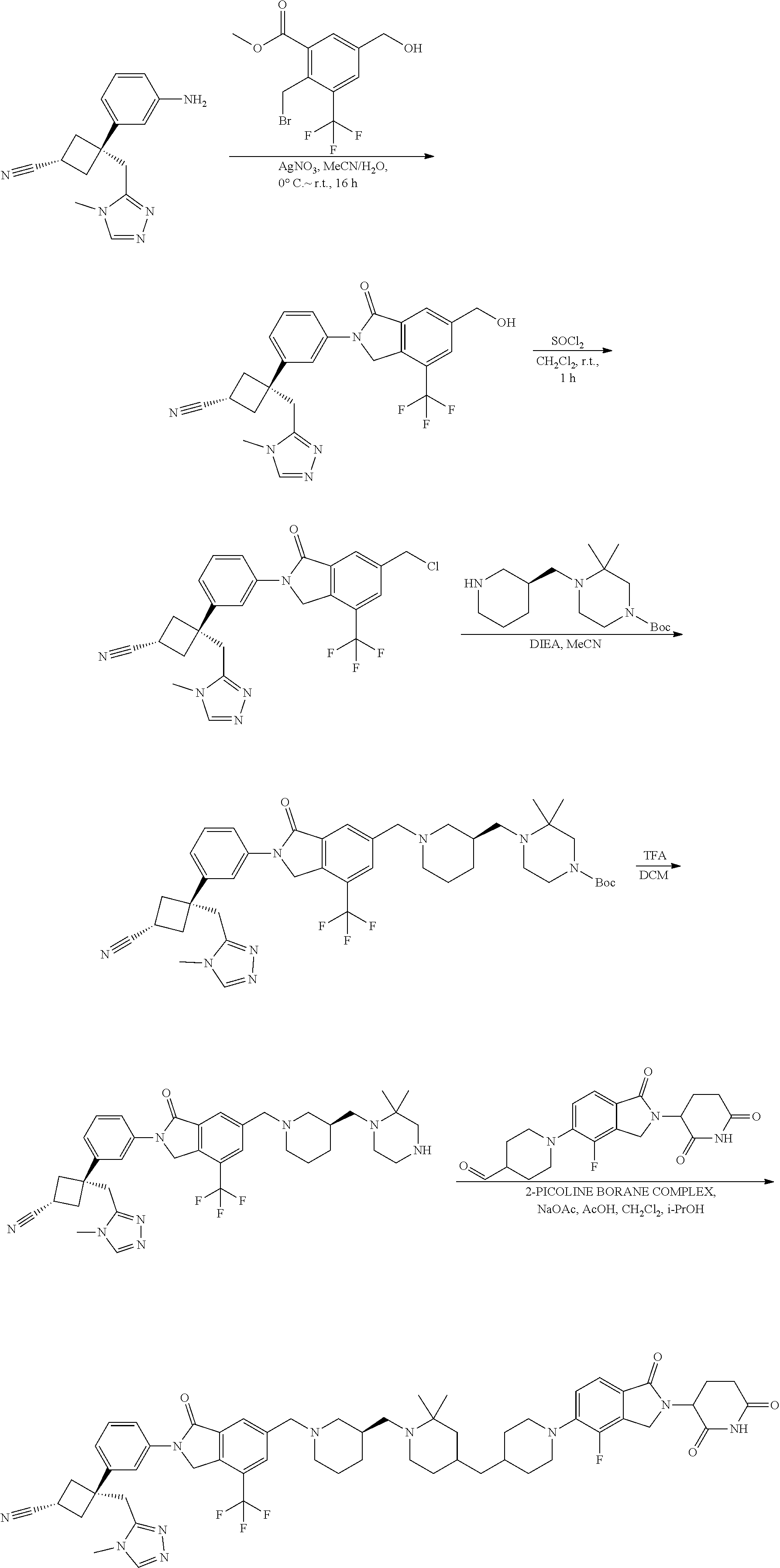

### Step 1: Preparation of 3-[3-[6-(hydroxymethyl)-1-oxo-4-(trifluoromethyl)isoindolin-2-yl]phenyl]-3-[(4-methyl-1,2,4-triazol-3-yl)methyl]cyclobutanecarbonitrile

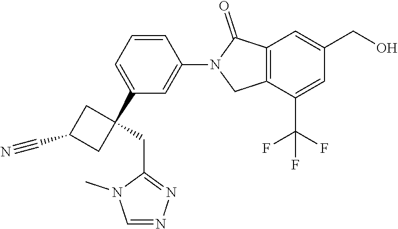 [SMILES: Cn1cnnc1C[C@]1(c2cccc(N3Cc4c(cc(CO)cc4C(F)(F)F)C3=O)c2)C[C@H](C#N)C1]

To a mixture of 3-(3-aminophenyl)-3-[(4-methyl-1,2,4-triazol-3-yl)methyl]cyclobutanecarbonitrile (332 mg, 1.2 mmol) and methyl 2-(bromomethyl)-5-(hydroxymethyl)-3-(trifluoromethyl)benzoate (386 mg, 1.2 mmol) in acetonitrile (20 mL) and water (10 mL) was added silver nitrate (310 mg, 1.8 mmol) at 0° C. The mixture was stirred at 20° C. for 16 h, then saturated sodium carbonate solution was added to adjust the pH of the mixture to 8-9. The resulting suspension was filtered and extracted with dichloromethane (30 mL×3). The combined organic layer was washed with brine (20 mL), dried over sodium sulfate, filtered, and concentrated. The residue was purified by flash column chromatography (0-6% methanol in dichloromethane) to afford 3-[3-[6-(hydroxymethyl)-1-oxo-4-(trifluoromethyl)isoindolin-2-yl]phenyl]-3-[(4-methyl-1,2,4-triazol-3-yl)methyl]cyclobutanecarbonitrile (420 mg, 70%) as light yellow solid. MS (ESI) m/z: 482.1 [M+H]⁺.

### Step 2: Preparation of 3-[3-[6-(chloromethyl)-1-oxo-4-(trifluoromethyl)isoindolin-2-yl]phenyl]-3-[(4-methyl-1,2,4-triazol-3-yl)methyl]cyclobutanecarbonitrile

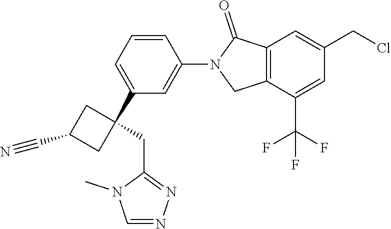 [SMILES: Cn1cnnc1C[C@]1(c2cccc(N3Cc4c(cc(CCl)cc4C(F)(F)F)C3=O)c2)C[C@H](C#N)C1]

To a solution of 3-[3-[6-(hydroxymethyl)-1-oxo-4-(trifluoromethyl)isoindolin-2-yl]phenyl]-3-[(4-methyl-1,2,4-triazol-3-yl)methyl]cyclobutanecarbonitrile (80 mg, 0.1 mmol) in dichloromethane (2.0 mL) was added thionyl chloride (57 μL, 0.8 mmol). The mixture was stirred at 20° C. for 1 h, then concentrated to afford 3-[3-[6-(chloromethyl)-1-oxo-4-(trifluoromethyl)isoindolin-2-yl]phenyl]-3-[(4-methyl-1,2,4-triazol-3-yl)methyl]cyclobutanecarbonitrile (80 mg, crude) as light yellow foam, which was used directly in the next step. MS (ESI) m/z: 500.2 [M+H]⁺.

### Step 3: Preparation of tert-butyl 4-[[(3S)-1-[[2-[3-[3-cyano-1-[(4-methyl-1,2,4-triazol-3-yl)methyl]cyclobutyl]phenyl]-3-oxo-7-(trifluoromethyl)isoindolin-5-yl]methyl]-3-piperidyl]methyl]-3,3-dimethyl-piperazine-1-carboxylate

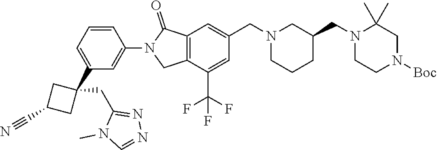 [SMILES: Cn1cnnc1C[C@]1(c2cccc(N3Cc4c(cc(CN5CCC[C@H](CN6CCN(C(=O)OC(C)(C)C)CC6(C)C)C5)cc4C(F)(F)F)C3=O)c2)C[C@H](C#N)C1]

To a mixture of tert-butyl 3,3-dimethyl-4-[[(3S)-3-piperidyl]methyl]piperazine-1-carboxylate (200 mg, 0.6 mmol) and N, N-diisopropylethylamine (248 mg, 1.9 mmol) in acetonitrile (5.0 mL) was added 3-[3-[6-(chloromethyl)-1-oxo-4-(trifluoromethyl)isoindolin-2-yl]phenyl]-3-[(4-methyl-1,2,4-triazol-3-yl)methyl]cyclobutanecarbonitrile (317 mg, 0.6 mmol). The mixture was stirred at 80° C. for 16 h, then concentrated. The residue was purified by flash column chromatography (0-5% methanol in dichloromethane) to afford tert-butyl 4-[[(3S)-1-[[2-[3-[3-cyano-1-[(4-methyl-1,2,4-triazol-3-yl)methyl]cyclobutyl]phenyl]-3-oxo-7-(trifluoromethyl)isoindolin-5-yl]methyl]-3-piperidyl]methyl]-3,3-dimethyl-piperazine-1-carboxylate (250 mg, 48%) as yellow oil. MS (ESI) m/z: 775.5 [M+H]⁺.

### Step 4: Preparation of 3-[3-[6-[[(3S)-3-[(2,2-dimethylpiperazin-1-yl)methyl]-1-piperidyl]methyl]-1-oxo-4-(trifluoromethyl)isoindolin-2-yl]phenyl]-3-[(4-methyl-1,2,4-triazol-3-yl)methyl]cyclobutanecarbonitrile

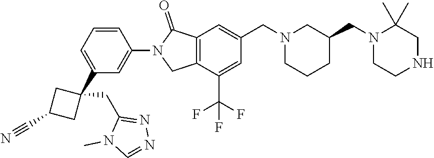 [SMILES: Cn1cnnc1C[C@]1(c2cccc(N3Cc4c(cc(CN5CCC[C@H](CN6CCNCC6(C)C)C5)cc4C(F)(F)F)C3=O)c2)C[C@H](C#N)C1]

To a mixture of tert-butyl 4-[[(3S)-1-[[2-[3-[3-cyano-1-[(4-methyl-1,2,4-triazol-3-yl)methyl]cyclobutyl]phenyl]-3-oxo-7-(trifluoromethyl)isoindolin-5-yl]methyl]-3-piperidyl]methyl]-3,3-dimethyl-piperazine-1-carboxylate (100 mg, 0.1 mmol) in dichloromethane (6.0 mL) was added trifluoroacetic acid (3.07 g, 27 mmol). The reaction was stirred at 25° C. for 30 min, then concentrated to afford 3-[3-[6-[[(3S)-3-[(2,2-dimethylpiperazin-1-yl)methyl]-1-piperidyl]methyl]-1-oxo-4-(trifluoromethyl)isoindolin-2-yl]phenyl]-3-[(4-methyl-1,2,4-triazol-3-yl)methyl]cyclobutanecarbonitrile trifluoroacetate (206 mg, crude) as yellow oil. MS (ESI) m/z: 675.4 [M+H]⁺.

### Step 5: Preparation of 3-[3-[6-[[(3S)-3-[[4-[[1-[2-(2,6-dioxo-3-piperidyl)-4-fluoro-1-oxoisoindolin-5-yl]-4-piperidyl]methyl]-2,2-dimethyl-piperazin-1-yl]methyl]-1-piperidyl]methyl]-1-oxo-4-(trifluoromethyl)isoindolin-2-yl]phenyl]-3-[(4-methyl-1,2,4-triazol-3-yl)methyl]cyclobutanecarbonitrile

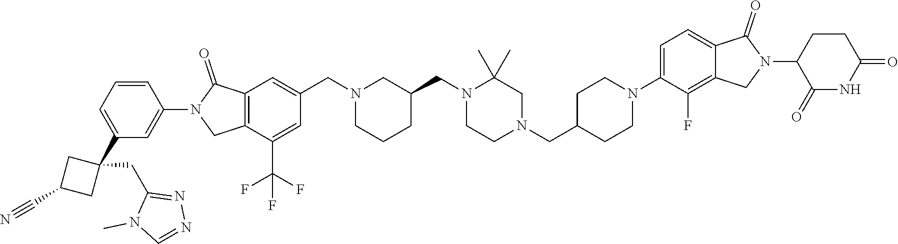 [SMILES: Cn1cnnc1C[C@]1(c2cccc(N3Cc4c(cc(CN5CCC[C@H](CN6CCN(CC7CCN(c8ccc9c(c8F)CN(C8CCC(=O)NC8=O)C9=O)CC7)CC6(C)C)C5)cc4C(F)(F)F)C3=O)c2)C[C@H](C#N)C1]

To a mixture of 3-[3-[6-[[(3S)-3-[(2,2-dimethylpiperazin-1-yl)methyl]-1-piperidyl]methyl]-1-oxo-4-(trifluoromethyl)isoindolin-2-yl]phenyl]-3-[(4-methyl-1,2,4-triazol-3-yl)methyl]cyclobutanecarbonitrile trifluoroacetate (206 mg, 0.1 mmol) and sodium acetate (107 mg, 1.3 mmol) in dichloromethane (10 mL) and isopropanol (10 mL) was added acetic acid (1.0 mL) and 1-[2-(2,6-dioxo-3-piperidyl)-4-fluoro-1-oxo-isoindolin-5-yl]piperidine-4-carbaldehyde (57 mg, 0.1 mmol). The mixture was stirred at 25° C. for 1 h, followed by the addition of 2-picoline borane (42 mg, 0.4 mmol). The reaction was stirred at 25° C. for 12 h, then concentrated under reduced pressure. The residue was purified by prep-HPLC (0-32% acetonitrile in water (0.2% formic acid) over 36 min) to afford 3-[3-[6-[[(3S)-3-[[4-[[1-[2-(2,6-dioxo-3-piperidyl)-4-fluoro-1-oxo-isoindolin-5-yl]-4-piperidyl]methyl]-2,2-dimethyl-piperazin-1-yl]methyl]-1-piperidyl]methyl]-1-oxo-4-(trifluoromethyl)isoindolin-2-yl]phenyl]-3-[(4-methyl-1,2,4-triazol-3-yl)methyl]cyclobutanecarbonitrile formate (58.7 mg, 44%) as white solid. MS (ESI) m/z: 1032.5 [M+H]⁺; ¹H NMR (400 MHz, DMSO-d₆) δ 10.97 (s, 1H), 8.16 (d, J=5.5 Hz, 2H), 7.96 (s, 1H), 7.91 (s, 1H), 7.86 (dd, J=1.6, 8.0 Hz, 1H), 7.45 (d, J=8.0 Hz, 2H), 7.34 (t, J=8.0 Hz, 1H), 7.14 (t, J=8.0 Hz, 1H), 6.83 (d, J=8.0 Hz, 1H), 5.10 (s, 2H), 5.08-5.04 (m, 1H), 4.47 (d, J=16.8 Hz, 1H), 4.30 (d, J=16.8 Hz, 1H), 3.66 (br d, J=13.2 Hz, 2H), 3.51-3.42 (m, 3H), 3.27-3.17 (m, 4H), 2.91-2.85 (m, 5H), 2.79 (s, 4H), 2.76-2.70 (m, 2H), 2.68-2.60 (m, 2H), 2.56 (br s, 1H), 2.37 (br d, J=2.8 Hz, 5H), 2.05 (br d, J=6.8 Hz, 3H), 2.00-1.93 (m, 2H), 1.77 (br d, J=12.0 Hz, 4H), 1.67-1.57 (m, 4H), 1.48-1.40 (m, 1H), 1.28-1.19 (m, 2H), 0.93 (br d, J=11.6 Hz, 6H).

### Example 154: Exemplary synthesis of 3-[3-[6-[7-[[1-[2-(2,6-dioxo-3-piperidyl)-4-fluoro-7-methyl-1-oxo-isoindolin-5-yl]-4-piperidyl]methyl]-2,7-diazaspiro[3.5]nonan-2-yl]-4-(trifluoromethyl)indazol-2-yl]phenyl]-3-[(4-methyl-1,2,4-triazol-3-yl)methyl]cyclobutanecarbonitrile

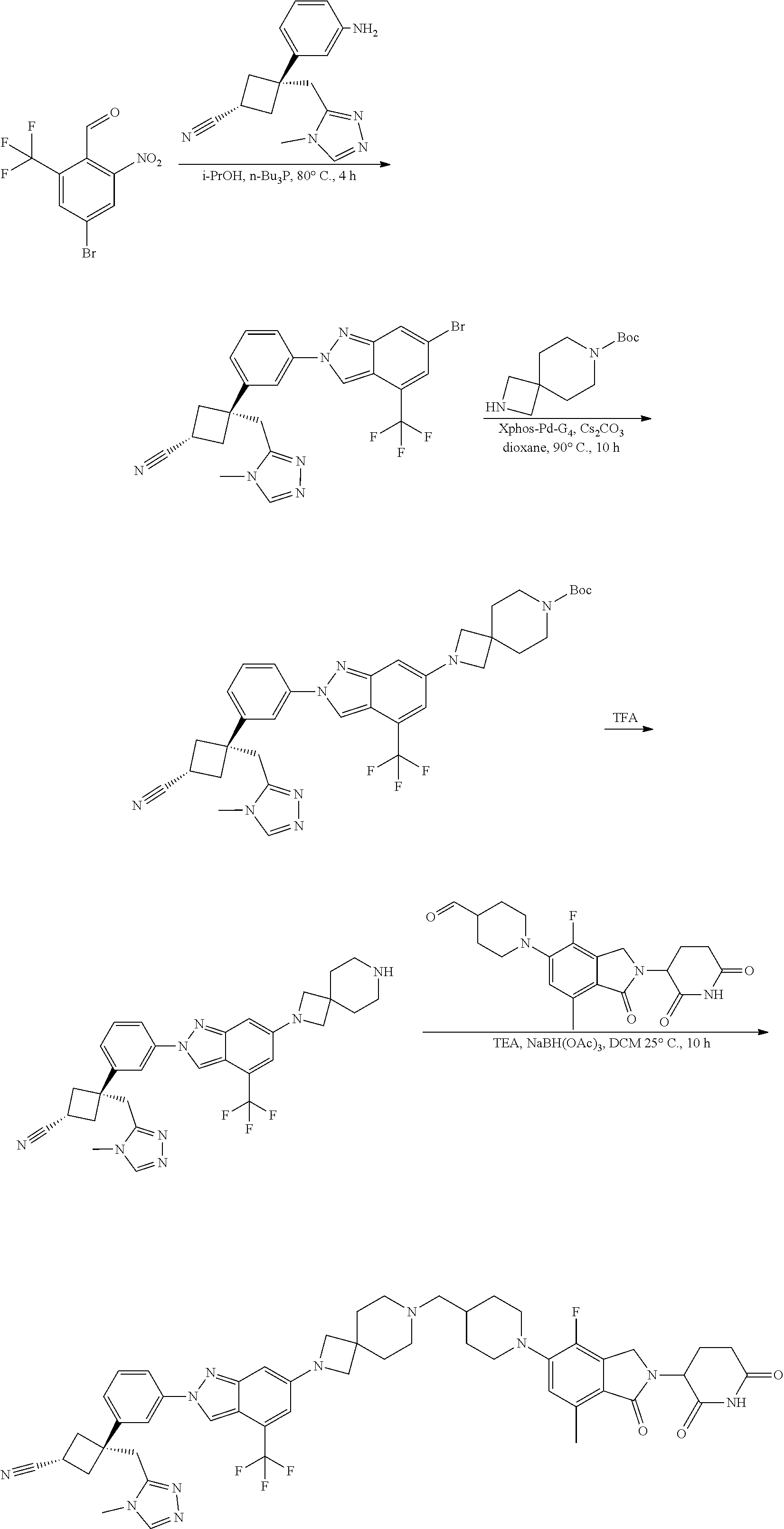

### Step 1: Preparation of 3-[3-[6-bromo-4-(trifluoromethyl)indazol-2-yl]phenyl]-3-[(4-methyl-1,2,4-triazol-3-yl)methyl]cyclobutanecarbonitrile

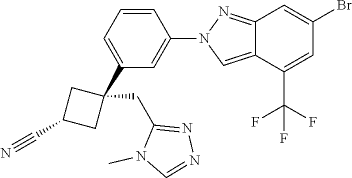 [SMILES: Cn1cnnc1C[C@]1(c2cccc(-n3cc4c(C(F)(F)F)cc(Br)cc4n3)c2)C[C@H](C#N)C1]

A mixture of 4-bromo-2-nitro-6-(trifluoromethyl)benzaldehyde (1.00 g, 3 mmol) and 3-(3-aminophenyl)-3-[(4-methyl-1,2,4-triazol-3-yl)methyl]cyclobutanecarbonitrile (897 mg, 3 mmol) in isopropanol (10 mL) was degassed and purged with nitrogen for three times, the mixture was stirred at 80° C. for 10 h under nitrogen atmosphere. Then tributylphosphane (2.04 g, 10 mmol) was added at 25° C. and the mixture was stirred at 80° C. for 10 h under nitrogen atmosphere. The mixture was filtered, washed with acetonitrile (8 mL) and concentrated under reduced pressure to afford 3-[3-[6-bromo-4-(trifluoromethyl)indazol-2-yl]phenyl]-3-[(4-methyl-1,2,4-triazol-3-yl)methyl]cyclobutanecarbonitrile (600 mg, 35%) as a white solid. MS (ESI) m/z: 517.2 [M+H]⁺; ¹H NMR (400 MHz, CDCl₃) δ 8.38 (s, 1H), 8.07 (s, 1H), 7.87 (s, 1H), 7.60-7.73 (m, 2H), 7.48 (s, 1H), 7.37 (t, J=8.0 Hz, 1H), 6.97 (d, J=8.0 Hz, 1H), 3.34 (s, 2H), 2.96-3.12 (m, 5H), 2.84 ppm (s, 3H).

### Step 2: Preparation of tert-butyl 2-[2-[3-[3-cyano-1-[(4-methyl-1,2,4-triazol-3-yl)methyl]cyclobutyl]phenyl]-4-(trifluoromethyl)indazol-6-yl]-2,7-diazaspiro[3.5]nonane-7-carboxylate

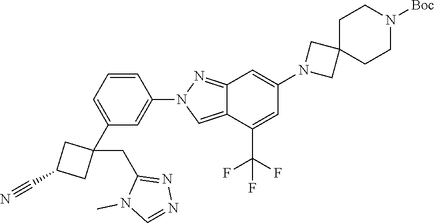

To a solution of 3-[3-[6-bromo-4-(trifluoromethyl)indazol-2-yl]phenyl]-3-[(4-methyl-1,2,4-triazol-3-yl)methyl]cyclobutanecarbonitrile (580 mg, 1 mmol), tert-butyl 2,7-diazaspiro[3.5]nonane-7-carboxylate (255 mg, 1 mmol) and cesium carbonate (1.10 g, 3 mmol) in 1,4-dioxane (1 mL) was added (sp-4-3)-[dicyclohexyl[2′,4′,6′-tris(1-methylethyl)[1,1′-biphenyl]-2-yl]phosphine](methanesulfonato-κO)[2′-(methylamino-κN)[1,1′-biphenyl]-2-yl-KC]palladium (194 mg, 0.2 mmol). The mixture was purged with nitrogen and stirred at 90° C. for 10 h under nitrogen. The mixture was filtered and concentrated under reduced pressure. The residue was purified by silica gel column chromatography (petroleum ether/ethyl acetate=1/1 to 0/1 then dichloromethane:methanol=30:1 to 10:1) to afford tert-butyl 2-[2-[3-[3-cyano-1-[(4-methyl-1,2,4-triazol-3-yl)methyl]cyclobutyl]phenyl]-4-(trifluoromethyl)indazol-6-yl]-2,7-diazaspiro[3.5]nonane-7-carboxylate (650 mg, 87%) as a yellow solid. MS (ESI) m/z: 661.4 [M+H]⁺.

### Step 3: Preparation of 3-[3-[6-(2,7-diazaspiro[3.5]nonan-2-yl)-4-(trifluoromethyl)indazol-2-yl]phenyl]-3-[(4-methyl-1,2,4-triazol-3-yl)methyl]cyclobutanecarbonitrile

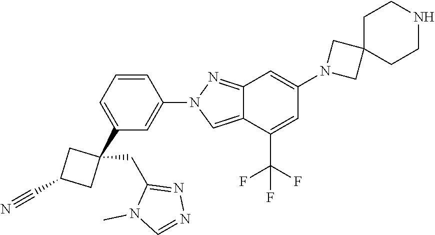 [SMILES: Cn1cnnc1C[C@]1(c2cccc(-n3cc4c(C(F)(F)F)cc(N5CC6(CCNCC6)C5)cc4n3)c2)C[C@H](C#N)C1]

To a solution of tert-butyl 2-[2-[3-[3-cyano-1-[(4-methyl-1,2,4-triazol-3-yl)methyl]cyclobutyl]phenyl]-4-(trifluoromethyl)indazol-6-yl]-2,7-diazaspiro[3.5]nonane-7-carboxylate (160 mg, 0.2 mmol) in dichloromethane (2 mL) was added trifluoroacetic acid (1.0 mL, 13 mmol). The mixture was stirred at 25° C. for 0.5 h, then concentrated under reduced pressure to afford 3-[3-[6-(2,7-diazaspiro[3.5]nonan-2-yl)-4-(trifluoromethyl)indazol-2-yl]phenyl]-3-[(4-methyl-1,2,4-triazol-3-yl)methyl]cyclobutanecarbonitrile formate (160 mg, 98%) as an off white oil. MS (ESI) m/z: 561.3 [M+H]⁺.

### Step 4: Preparation of 3-[3-[6-[7-[[1-[2-(2,6-dioxo-3-piperidyl)-4-fluoro-7-methyl-1-oxo-isoindolin-5-yl]-4-piperidyl]methyl]-2,7-diazaspiro[3.5]nonan-2-yl]-4-(trifluoromethyl)indazol-2-yl]phenyl]-3-[(4-methyl-1,2,4-triazol-3-yl)methyl]cyclobutanecarbonitrile

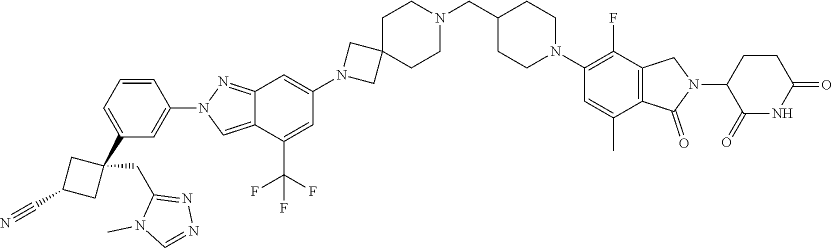 [SMILES: Cc1cc(N2CCC(CN3CCC4(CC3)CN(c3cc(C(F)(F)F)c5cn(-c6cccc([C@]7(Cc8nncn8C)C[C@@H](C#N)C7)c6)nc5c3)C4)CC2)c(F)c2c1C(=O)N(C1CCC(=O)NC1=O)C2]

A solution of 3-[3-[6-(2,7-diazaspiro[3.5]nonan-2-yl)-4-(trifluoromethyl)indazol-2-yl]phenyl]-3-[(4-methyl-1,2,4-triazol-3-yl)methyl]cyclobutanecarbonitrile formate (160 mg, 0.2 mmol), 1-[2-(2,6-dioxo-3-piperidyl)-4-fluoro-7-methyl-1-oxo-isoindolin-5-yl]piperidine-4-carbaldehyde (92 mg, 0.2 mmol) and triethylamine (24 mg, 0.2 mmol) in dichloromethane (2 mL) was stirred at 25° C. for 10 h. Then sodium triacetoxyborohydride (151 mg, 0.7 mmol) was added and the mixture was stirred at 25° C. for 1 h. The reaction was concentrated under reduced pressure. The residue was purified by prep-HPLC (15%-45% acetonitrile in water (formic acid) over 10 min) to afford 3-[3-[6-[7-[[1-[2-(2,6-dioxo-3-piperidyl)-4-fluoro-7-methyl-1-oxo-isoindolin-5-yl]-4-piperidyl]methyl]-2,7-diazaspiro[3.5]nonan-2-yl]-4-(trifluoromethyl)indazol-2-yl]phenyl]-3-[(4-methyl-1,2,4-triazol-3-yl)methyl]cyclobutanecarbonitrile (60.2 mg, 27%) as a white solid. MS (ESI) m/z: 932.6 [M+H]⁺; ¹H NMR (400 MHz, DMSO-d₆) δ 10.97 (s, 1H), 9.06 (s, 1H), 8.19 (s, 1H), 7.98 (dd, J=1.2, 8.4 Hz, 1H), 7.84 (s, 1H), 7.44 (t, J=8.0 Hz, 1H), 7.01 (d, J=7.6 Hz, 1H), 6.94-6.86 (m, 2H), 6.63 (s, 1H), 5.03 (dd, J=4.8, 13.4 Hz, 1H), 4.40 (d, J=16.8 Hz, 1H), 4.24 (d, J=16.8 Hz, 1H), 3.69 (s, 4H), 3.54-3.47 (m, 2H), 3.37 (br s, 2H), 3.26 (br d, J=7.6 Hz, 2H), 2.97-2.90 (m, 4H), 2.88 (s, 3H), 2.77 (br t, J=11.2 Hz, 2H), 2.61 (br s, 6H), 2.48-2.36 (m, 4H), 2.20 (br d, J=7.2 Hz, 2H), 1.99-1.93 (m, 1H), 1.84-1.77 (m, 5H), 1.71 (br d, J=4.4 Hz, 1H), 1.37-1.21 (m, 2H).

### Example 155: Exemplary synthesis of 3-[4-fluoro-5-[4-[[(3S)-1-[[(3R)-1-[[2-[3-[3-[(4-methyl-1,2,4-triazol-3-yl)methyl]oxetan-3-yl]phenyl]-4-(trifluoromethyl)indazol-6-yl]methyl]-3-piperidyl]methyl]-3-piperidyl]methyl]piperazin-1-yl]-1-oxo-isoindolin-2-yl]piperidine-2,6-dione

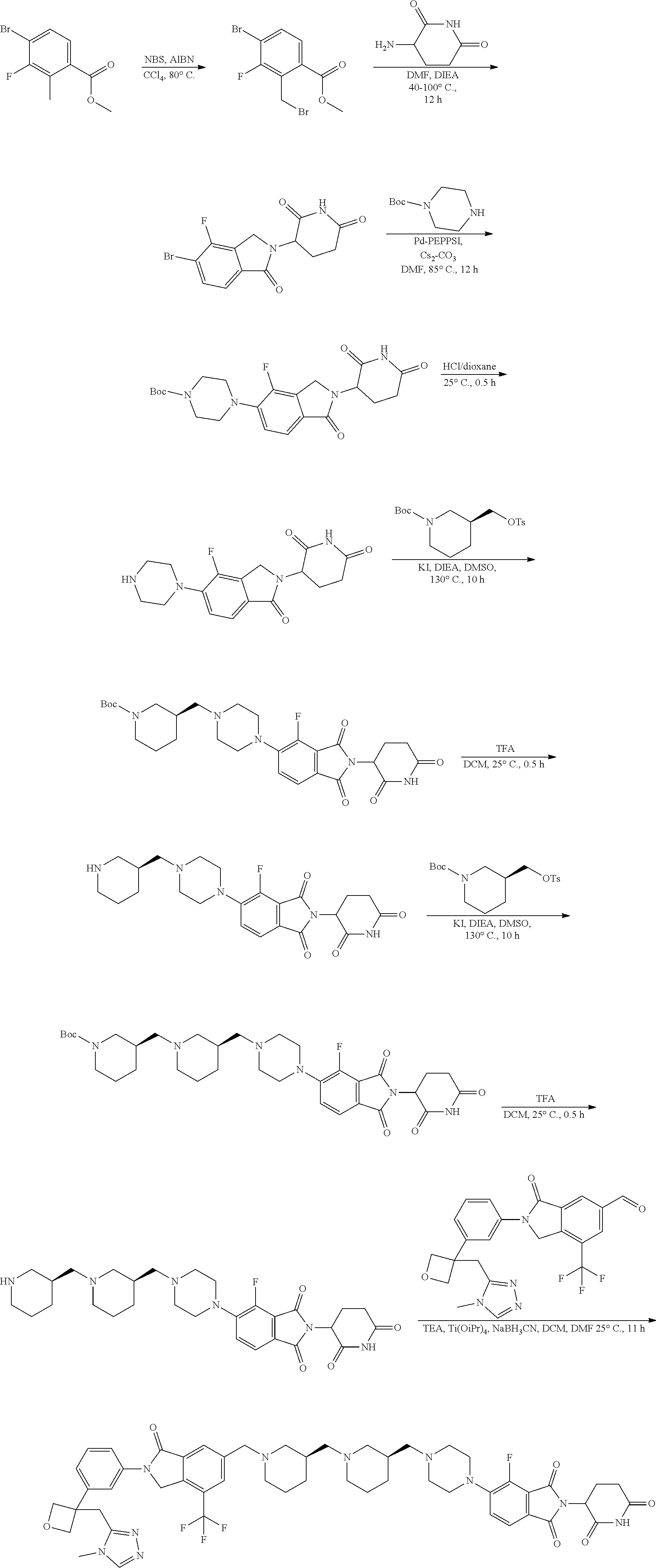

### Step 1: Preparation of methyl 4-bromo-2-(bromomethyl)-3-fluorobenzoate

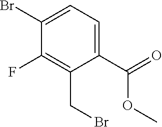

To a solution of methyl 4-bromo-3-fluoro-2-methyl-benzoate (2 g, 8 mmol) in carbon tetrachloride (20 mL) was added azobisisobutyronitrile (133 mg, 0.8 mmol) and N-bromosuccinimide (1.44 g, 8 mmol). The mixture was stirred at 80° C. for 16 h, then diluted with water (30 mL) and extracted with dichloromethane (20 mL×3). The combined organic phase was dried over sodium sulfate, filtered, and concentrated under reduced pressure. The residue was purified by column chromatography (petroleum ether/ethyl acetate=10/1 to 6/1) to afford methyl 4-bromo-2-(bromomethyl)-3-fluoro-benzoate (2.3 g, 87%) as a yellow solid. ¹H NMR (400 MHz, CDCl₃) δ 7.71-7.69 (m, 1H), 7.74-7.54 (m, 1H), 5.09-4.89 (m, 2H), 3.98 (s, 3H).

### Step 2: Preparation of 3-(5-bromo-4-fluoro-1-oxoisoindolin-2-yl)piperidine-2,6-dione

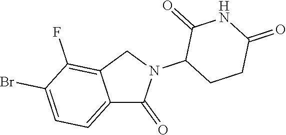 [SMILES: O=C1CCC(N2Cc3c(ccc(Br)c3F)C2=O)C(=O)N1]

To a solution of 3-aminopiperidine-2,6-dione hydrochloride (904 mg, 5 mmol) in N, N-dimethylformamide (25 mL) was added N, N-diisopropylethylamine (6.15 mL, 35 mmol). The reaction mixture was stirred for 30 min, then methyl 4-bromo-2-(bromomethyl)-3-fluoro-benzoate (2.3 g, 7 mmol) was added. The mixture was stirred at 100° C. for 11.5 h, then poured into cold water (60 mL). The suspension was filtered, and the filtrate cake triturated with petroleum ether and methyl tert-butyl ether (V/V=1:1, 20 mL) to afford 3-(5-bromo-4-fluoro-1-oxo-isoindolin-2-yl)piperidine-2,6-dione (1.4 g, 58%) as a gray solid. MS (ESI) m/z: 341.0 [M+H]⁺; ¹H NMR (400 MHz, DMSO-d₆) δ 11.03 (s, 1H), 7.89 (dd, J=6.0, 8.0 Hz, 1H), 7.56 (d, J=8.0 Hz, 1H), 5.13 (dd, J=5.2, 13.2 Hz, 1H), 4.73-4.05 (m, 2H), 2.90 (s, 1H), 2.44 (br dd, J=4.4, 12.8 Hz, 2H), 2.09-1.90 (m, 1H).

### Step 3: Preparation of tert-butyl 4-[2-(2,6-dioxo-3-piperidyl)-4-fluoro-1-oxo-isoindolin-5-yl]piperazine-1-carboxylate

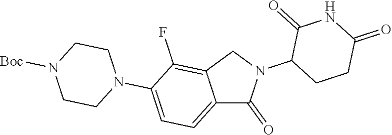 [SMILES: CC(C)(C)OC(=O)N1CCN(c2ccc3c(c2F)CN(C2CCC(=O)NC2=O)C3=O)CC1]

To a mixture of 3-(5-bromo-4-fluoro-1-oxo-isoindolin-2-yl)piperidine-2,6-dione (2.00 g, 6 mmol) and tert-butyl piperazine-1-carboxylate (1.20 g, 6 mmol) in N, N-dimethylformamide (30 mL) was added 1,3-bis[2,6-bis(1-ethylpropyl)phenyl]-2H-imidazole; 3-chloropyridine; dichloropalladium (1.14 g, 1 mmol) and cesium carbonate (3.82 g, 12 mmol). The mixture was degassed and purged with nitrogen then stirred under nitrogen at 85° C. for 12 h. The mixture was filtered, and the filtrate solution was diluted with 1 N aqueous hydrochloride acid solution (50 mL) and extracted with ethyl acetate (50 mL×3). The combined organic layers were concentrated under reduced pressure. The residue was purified by silica gel column chromatography (petroleum ether:ethyl acetate=1:1 to 0:1) to afford tert-butyl 4-[2-(2,6-dioxo-3-piperidyl)-4-fluoro-1-oxo-isoindolin-5-yl]piperazine-1-carboxylate (0.95 g, 36%) as an off-white solid. MS (ESI) m/z: 447.3 [M+H]⁺.

### Step 4: Preparation of 3-(4-fluoro-1-oxo-5-piperazin-1-yl-isoindolin-2-yl) piperidine-2,6-dione

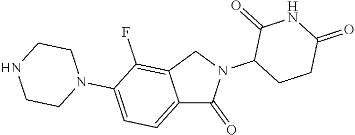 [SMILES: O=C1CCC(N2Cc3c(ccc(N4CCNCC4)c3F)C2=O)C(=O)N1]

A solution of tert-butyl 4-[2-(2,6-dioxo-3-piperidyl)-4-fluoro-1-oxo-isoindolin-5-yl]piperazine-1-carboxylate (0.35 g, 0.8 mmol) in 4 M hydrochloric acid in 1,4-dioxane (10 mL) was stirred at 25° C. for 0.5 h. The reaction was concentrated under reduced pressure to afford 3-(4-fluoro-1-oxo-5-piperazin-1-yl-isoindolin-2-yl)piperidine-2,6-dione hydrochloride (0.30 g, 99%) as a yellow solid.

### Step 5: Preparation of tert-butyl (3R)-3-[[4-[2-(2,6-dioxo-3-piperidyl)-4-fluoro-1-oxo-isoindolin-5-yl]piperazin-1-yl]methyl]piperidine-1-carboxylate

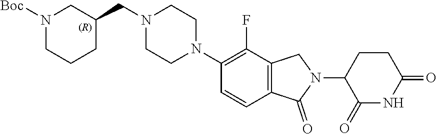

To a solution of tert-butyl (3S)-3-(p-tolylsulfonyloxymethyl)piperidine-1-carboxylate (2.13 g, 5.8 mmol) and 3-(4-fluoro-1-oxo-5-piperazin-1-yl-isoindolin-2-yl)piperidine-2,6-dione (1.00 g, 3 mmol) in dimethyl sulfoxide (15 mL) was added N,N-diisopropylethylamine (1.12 g, 9 mmol) and potassium iodide (1.44 g, 9 mmol). The mixture was stirred at 130° C. for 10 h, then diluted with water (100 mL) and extracted with ethyl acetate (30 mL×3). The organic phase was washed with brine (100 mL), dried over anhydrous sodium sulfate, filtered, and concentrated. The residue was purified by column chromatography (petroleum ether/ethyl acetate=1/1 then dichloromethane:methanol=30:1) to afford tert-butyl (3R)-3-[[4-[2-(2,6-dioxo-3-piperidyl)-4-fluoro-1-oxo-isoindolin-5-yl]piperazin-1-yl]methyl]piperidine-1-carboxylate (900 mg, 57%) as a yellow solid. MS (ESI) m/z: 544.4 [M+H]⁺; ¹H NMR (400 MHz, DMSO) δ 10.97 (s, 1H), 7.49 (d, J=8.0 Hz, 1H), 7.28-7.09 (m, 1H), 5.08 (dd, J=5.2, 13.2 Hz, 1H), 4.63-4.42 (m, 1H), 4.39-4.26 (m, 1H), 4.01-3.86 (m, 1H), 3.78 (d, J=11.6 Hz, 1H), 3.23-3.04 (m, 4H), 3.00-2.85 (m, 1H), 2.78 (t, J=10.8 Hz, 1H), 2.69-2.54 (m, 4H), 2.49-2.31 (m, 4H), 2.27-2.10 (m, 2H), 1.80-1.54 (m, 3H), 1.40 (s, 9H), 1.33 (d, J=3.2 Hz, 1H), 1.13 (dd, J=7.2, 8.8 Hz, 1H).

### Step 6: Preparation of 3-[4-fluoro-1-oxo-5-[4-[[(3S)-3-piperidyl]methyl]piperazin-1-yl]isoindolin-2-yl]piperidine-2,6-dione

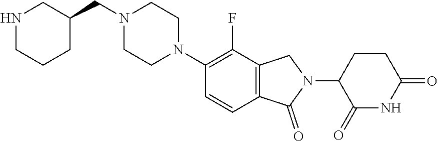 [SMILES: O=C1CCC(N2Cc3c(ccc(N4CCN(C[C@H]5CCCNC5)CC4)c3F)C2=O)C(=O)N1]

To a solution of tert-butyl (3R)-3-[[4-[2-(2,6-dioxo-3-piperidyl)-4-fluoro-1-oxo-isoindolin-5-yl]piperazin-1-yl]methyl]piperidine-1-carboxylate (500 mg, 0.9 mmol) in dichloromethane (1 mL) was added trifluoroacetic acid (461 mg, 4 mmol). The mixture was stirred at 25° C. for 0.5 h, then concentrated under reduced pressure to afford 3-[4-fluoro-1-oxo-5-[4-[[(3S)-3-piperidyl]methyl]piperazin-1-yl]isoindolin-2-yl]piperidine-2,6-dione trifluoroacetate (512 mg, 99%) as a dark brown oil, which was used in the next step directly. MS (ESI) m/z: 444.2 [M+H]⁺.

### Step 7: Preparation of tert-butyl (3R)-3-[[(3S)-3-[[4-[2-(2,6-dioxo-3-piperidyl)-4-fluoro-1-oxo-isoindolin-5-yl]piperazin-1-yl]methyl]-1-piperidyl]methyl]piperidine-1-carboxylate

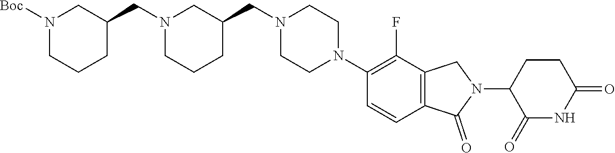 [SMILES: CC(C)(C)OC(=O)N1CCC[C@H](CN2CCC[C@H](CN3CCN(c4ccc5c(c4F)CN(C4CCC(=O)NC4=O)C5=O)CC3)C2)C1]

To a solution of 3-[4-fluoro-1-oxo-5-[4-[[(3S)-3-piperidyl]methyl]piperazin-1-yl]isoindolin-2-yl]piperidine-2,6-dione trifluoroacetate (500 mg, 0.90 mmol) and N,N-diisopropylethylamine (348 mg, 2.7 mmol) in dimethyl sulfoxide (8 mL) was added tert-butyl (3S)-3-(p-tolylsulfonyloxymethyl)piperidine-1-carboxylate (663 mg, 1.8 mmol) and potassium iodide (447 mg, 2.7 mmol). The mixture was stirred at 130° C. for 10 h, then diluted with water (50 mL) and extracted with ethyl acetate (15 mL×3). The combined organic phase was washed with brine (50 mL), dried over anhydrous sodium sulfate, filtered, and concentrated. The residue was purified by column chromatography on silica gel (petroleum ether/ethyl acetate=1/1 then dichloromethane:methanol=30/1) to afford tert-butyl (3R)-3-[[(3S)-3-[[4-[2-(2,6-dioxo-3-piperidyl)-4-fluoro-1-oxo-isoindolin-5-yl]piperazin-1-yl]methyl]-1-piperidyl]methyl]piperidine-1-carboxylate (300 mg, 52%) as a dark brown oil. MS (ESI) m/z: 641.5 [M+H]⁺.

### Step 8: Preparation of 3-[4-fluoro-1-oxo-5-[4-[[(3S)-1-[[(3S)-3-piperidyl]methyl]-3-piperidyl]methyl]piperazin-1-yl]isoindolin-2-yl]piperidine-2,6-dione

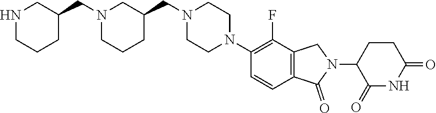 [SMILES: O=C1CCC(N2Cc3c(ccc(N4CCN(C[C@H]5CCCN(C[C@H]6CCCNC6)C5)CC4)c3F)C2=O)C(=O)N1]

To a solution of tert-butyl (3R)-3-[[(3S)-3-[[4-[2-(2,6-dioxo-3-piperidyl)-4-fluoro-1-oxo-isoindolin-5-yl]piperazin-1-yl]methyl]-1-piperidyl]methyl]piperidine-1-carboxylate (140 mg, 0.2 mmol) in dichloromethane (1 mL) was added trifluoroacetic acid (461 mg, 4 mmol). The mixture was stirred at 25° C. for 0.5 h, then concentrated under reduced pressure to afford 3-[4-fluoro-1-oxo-5-[4-[[(3S)-1-[[(3S)-3-piperidyl]methyl]-3-piperidyl]methyl]piperazin-1-yl]isoindolin-2-yl]piperidine-2,6-dione trifluoroacetate (143 mg, 99%) as a dark brown oil, which was used in the next step directly. MS (ESI) m/z: 541.5 [M+H]⁺.

### Step 9: Preparation of 3-[4-fluoro-5-[4-[[(3S)-1-[[(3R)-1-[[2-[3-[3-[(4-methyl-1,2,4-triazol-3-yl)methyl]oxetan-3-yl]phenyl]-4-(trifluoromethyl)indazol-6-yl]methyl]-3-piperidyl]methyl]-3-piperidyl]methyl]piperazin-1-yl]-1-oxo-isoindolin-2-yl]piperidine-2,6-dione

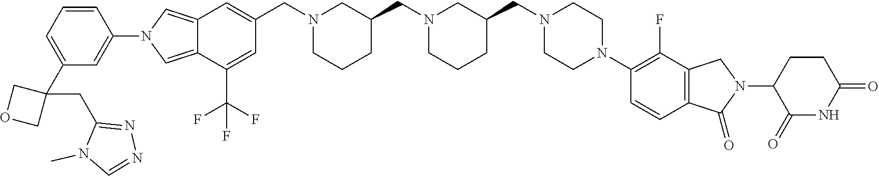 [SMILES: Cn1cnnc1CC1(c2cccc(-n3cc4cc(CN5CCC[C@H](CN6CCC[C@H](CN7CCN(c8ccc9c(c8F)CN(C8CCC(=O)NC8=O)C9=O)CC7)C6)C5)cc(C(F)(F)F)c4c3)c2)COC1]

To a solution of 3-[4-fluoro-1-oxo-5-[4-[[(3S)-1-[[(3S)-3-piperidyl]methyl]-3-piperidyl]methyl]piperazin-1-yl]isoindolin-2-yl]piperidine-2,6-dione trifluoroacetate (140 mg, 0.2 mmol) and 2-[3-[3-[(4-methyl-1,2,4-triazol-3-yl)methyl]oxetan-3-yl]phenyl]-4-(trifluoromethyl)indazole-6-carbaldehyde (95 mg, 0.2 mmol) in dichloromethane (0.5 mL) and N, N-dimethylformamide (0.5 mL) was added triethylamine (65 mg, 0.6 mmol) and tetraisopropoxytitanium (61 mg, 0.2 mmol). The mixture was stirred at 25° C. for 10 h, followed by the addition of sodium cyanoborohydride (41 mg, 0.6 mmol), the mixture was stirred at 25° C. for 1 h. The reaction was diluted with water (10 mL) and extracted with dichloromethane (5 mL×3). The combined organic phase washed with brine (10 mL), dried over anhydrous sodium sulfate, filtered, and concentrated. The residue was purified by prep-HPLC (5%-35% acetonitrile in water (formic acid) over 10 min) to afford 3-[4-fluoro-5-[4-[[(3S)-1-[[(3R)-1-[[2-[3-[3-[(4-methyl-1,2,4-triazol-3-yl)methyl]oxetan-3-yl]phenyl]-4-(trifluoromethyl)indazol-6-yl]methyl]-3-piperidyl]methyl]-3-piperidyl]methyl]piperazin-1-yl]-1-oxo-isoindolin-2-yl]piperidine-2,6-dione formate (23.4 mg, 10%) as a yellow resin. MS (ESI) m/z: 966.6 [M+H]⁺; ¹H NMR (400 MHz, DMSO-d₆) δ 11.00 (s, 1H), 9.23 (s, 1H), 8.20 (s, 1H), 8.14 (s, 1H), 8.07-8.00 (m, 1H), 7.94 (s, 1H), 7.88 (s, 1H), 7.56 (s, 1H), 7.50-7.39 (m, 2H), 7.13-6.96 (m, 2H), 5.13-5.04 (m, 1H), 5.00-4.93 (m, 4H), 4.52-4.25 (m, 2H), 3.76-3.66 (m, 2H), 3.64-3.52 (m, 3H), 3.06 (d, J=2.0 Hz, 4H), 3.04-3.01 (m, 3H), 2.93-2.87 (m, 2H), 2.76 (d, J=4.0, 7.6 Hz, 2H), 2.62 (d, J=3.6 Hz, 2H), 2.58-2.56 (m, 2H), 2.44 (d, J=1.6 Hz, 2H), 2.41 (s, 2H), 2.38-2.36 (m, 1H), 2.20-2.10 (m, 4H), 2.01-1.88 (m, 4H), 1.72-1.61 (m, 4H), 1.58-1.46 (m, 2H), 1.07-0.96 (m, 2H).

### Example 276: Exemplary synthesis of 3-(5-(4-(((1S,4r)-4-((3,3-dimethyl-4-(((3S)-1-((1-methyl-2-(3-(3-((4-methyl-4H-1,2,4-triazol-3-yl)methyl)oxetan-3-yl)phenyl)-3-oxo-7-(trifluoromethyl)isoindolin-5-yl)methyl)piperidin-3-yl)methyl)piperazin-1-yl)methyl)cyclohexyl)oxy)piperidin-1-yl)-4-fluoro-1-oxoisoindolin-2-yl)piperidine-2,6-dione

### Step 1: Preparation of methyl 2-iodo-3-(trifluoromethyl)benzoate

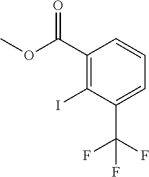

To a solution of methyl 2-amino-3-(trifluoromethyl)benzoate (4.0 g, 18 mmol) in acetonitrile (40 mL) and water (20 mL) was added tosylic acid monohydrate (10.4 g, 55 mmol) at 0° C. The reaction mixture was stirred for 10 min, then sodium nitrite (2.5 g, 37 mmol) was added and the mixture was stirred for 10 min before the addition of potassium iodide (7.57 g, 46 mmol). The reaction mixture was stirred at 20° C. for 16 h. The pH of the mixture was adjusted to 8-9 by 1 M aqueous sodium hydroxide solution, and the resulting mixture was diluted with water (50 mL) and extracted with dichloromethane (80 mL×3). The combined organic layer was washed with brine (100 mL), dried over sodium sulfate, filtered, and concentrated. The residue was purified by flash column chromatography (0-4% ethyl acetate in petroleum ether) to afford methyl 2-iodo-3-(trifluoromethyl)benzoate (4.39 g, 73%) as colorless oil. ¹H NMR (400 MHz, CDCl₃) δ 7.74 (br d, J=7.6 Hz, 1H), 7.63-7.58 (m, 1H), 7.54-7.49 (m, 1H), 3.99 (s, 3H).

### Step 2: Preparation of methyl 3-(trifluoromethyl)-2-vinyl-benzoate

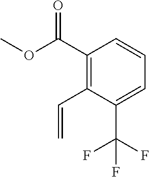 [SMILES: C=Cc1c(C(=O)OC)cccc1C(F)(F)F]

To a mixture of methyl 2-iodo-3-(trifluoromethyl)benzoate (1.35 g, 3.8 mmol) and potassium vinyltrifluoroborate (1.53 g, 11 mmol) in 1,4-dioxane (20 mL) and water (4.0 mL) were added sodium bicarbonate (1.01 g, 9.5 mmol) and Pd(dppf)Cl₂·CH₂Cl₂ (310 mg, 0.4 mmol). The reaction mixture was stirred at 110° C. for 16 h under nitrogen, then filtered and concentrated. The residue was purified by flash column chromatography (0-3% ethyl acetate in petroleum ether) to afford methyl 3-(trifluoromethyl)-2-vinyl-benzoate (378 mg, 38%) as colorless oil. MS (ESI) m/z: 231.0 [M+H]⁺; ¹H NMR (400 MHz, CDCl₃) δ 7.84 (d, J=7.6 Hz, 1H), 7.79 (d, J=8.0 Hz, 1H), 7.44 (t, J=7.6 Hz, 1H), 7.16-7.05 (m, 1H), 5.49 (dd, J=0.8, 11.6 Hz, 1H), 5.26 (d, J=17.6 Hz, 1H), 3.87 (s, 3H).

### Step 3: Preparation of methyl 2-ethyl-3-(trifluoromethyl)benzoate

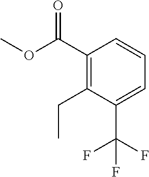 [SMILES: CCc1c(C(=O)OC)cccc1C(F)(F)F]

To a solution of methyl 3-(trifluoromethyl)-2-vinyl-benzoate (2.3 g, 10 mmol) in methanol (20 mL) was added 10% palladium on carbon (500 mg). The mixture was degassed under vacuum and purged with hydrogen several times. The mixture was stirred under hydrogen (15 psi) at 25° C. for 16 h. The reaction was filtered, the filter cake was washed with ethyl acetate (10 mL×3), and the filtrate was concentrated to afford methyl 2-ethyl-3-(trifluoromethyl)benzoate (1.82 g, crude) as colorless oil. ¹H NMR (400 MHz, CDCl₃) δ 7.92 (d, J=7.6 Hz, 1H), 7.79 (d, J=7.6 Hz, 1H), 7.35 (t, J=7.6 Hz, 1H), 3.94 (s, 3H), 3.13 (q, J=7.2 Hz, 2H), 1.22 (t, J=7.2 Hz, 3H).

### Step 4: Preparation of methyl 5-bromo-2-ethyl-3-(trifluoromethyl)benzoate

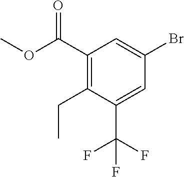 [SMILES: CCc1c(C(=O)OC)cc(Br)cc1C(F)(F)F]

To a solution of methyl 2-ethyl-3-(trifluoromethyl)benzoate (1.82 g, 7 mmol) in acetic acid (20 mL) were slowly added nitric acid (3.5 mL, 78 mmol), bromine (436 μL, 8.5 mmol) and silver nitrate (4.9 mL, 2.5 M in water). The resulting mixture was stirred at 25° C. for 2 h. The mixture was diluted with water (50 mL) and extracted with ethyl acetate (50 mL×3). The combined organic layers were washed with saturated sodium bicarbonate (80 mL×3) then brine (80 mL), dried over sodium sulfate, filtered, and concentrated. The residue was purified by flash column chromatography (petroleum ether) to afford methyl 5-bromo-2-ethyl-3-(trifluoromethyl)benzoate (1.52 g, 63%) as colorless oil. ¹H NMR (400 MHz, CDCl₃) δ 8.07 (d, J=2.0 Hz, 1H), 7.91 (d, J=2.0 Hz, 1H), 3.94 (s, 3H), 3.07 (q, J=7.2 Hz, 2H), 1.20 (t, J=7.2 Hz, 3H).

### Step 5: Preparation of methyl 5-bromo-2-(1-bromoethyl)-3-(trifluoromethyl)benzoate

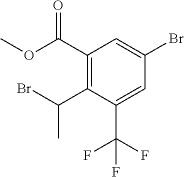 [SMILES: COC(=O)c1cc(Br)cc(C(F)(F)F)c1C(C)Br]

To a solution of methyl 5-bromo-2-ethyl-3-(trifluoromethyl)benzoate (1.52 g, 4.5 mmol) and NBS (1.19 g, 6.7 mmol) in 1,2-dichloroethane (20 mL) was added AIBN (146 mg, 0.9 mmol) at 0° C., and the mixture was stirred at 60° C. for 16 h under nitrogen atmosphere. The mixture was concentrated, the residue was suspended in petroleum ether (20 mL) and filtered, and the filtrate was concentrated to afford methyl 5-bromo-2-(1-bromoethyl)-3-(trifluoromethyl)benzoate (1.93 g, crude) as colorless oil. ¹H NMR (400 MHz, CDCl₃) δ 7.86 (s, 1H), 7.82 (d, J=1.6 Hz, 1H), 5.61 (q, J=7.2 Hz, 1H), 3.98 (s, 3H), 2.17 (d, J=7.2 Hz, 3H).

### Step 6: Preparation of 6-bromo-3-methyl-2-[3-[3-[(4-methyl-1,2,4-triazol-3-yl)methyl]oxetan-3-yl]phenyl]-4-(trifluoromethyl)isoindolin-1-one

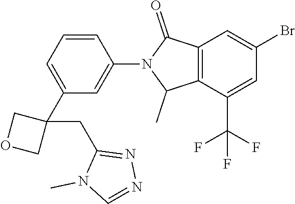 [SMILES: CC1c2c(cc(Br)cc2C(F)(F)F)C(=O)N1c1cccc(C2(Cc3nncn3C)COC2)c1]

To a mixture of methyl 5-bromo-2-(1-bromoethyl)-3-(trifluoromethyl)benzoate (1.93 g, 4.5 mmol) and 3-[3-[(4-methyl-1,2,4-triazol-3-yl)methyl]oxetan-3-yl]aniline (1.09 g, 4.5 mmol) in acetonitrile (30 mL) and water (15 mL) was added silver nitrate (984 mg, 5.8 mmol). The mixture was stirred at 60° C. for 16 h before the addition of TFA (331 μL, 4.5 mmol), and the reaction mixture was stirred at 60° C. for 5 h. The mixture was diluted with water (60 mL), and the pH of the mixture was adjusted to 8-9 with solid sodium bicarbonate. The resulting mixture was filtered; the filtrate was lyophilized and the residue was purified by flash column chromatography (0-20% dichloromethane in petroleum ether, then 0-8% methanol in dichloromethane) to afford 6-bromo-3-methyl-2-[3-[3-[(4-methyl-1,2,4-triazol-3-yl)methyl]oxetan-3-yl]phenyl]-4-(trifluoromethyl)isoindolin-1-one (301 mg, 12%) as a yellow solid.

### Step 7: Preparation of 3-methyl-2-[3-[3-[(4-methyl-1,2,4-triazol-3-yl)methyl]oxetan-3-yl]phenyl]-4-(trifluoromethyl)-6-vinyl-isoindolin-1-one

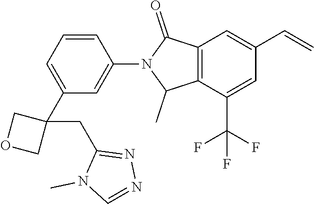 [SMILES: C=Cc1cc2c(c(C(F)(F)F)c1)C(C)N(c1cccc(C3(Cc4nncn4C)COC3)c1)C2=O]

To a mixture of 6-bromo-3-methyl-2-[3-[3-[(4-methyl-1,2,4-triazol-3-yl)methyl]oxetan-3-yl]phenyl]-4-(trifluoromethyl)isoindolin-1-one (301 mg, 0.5 mmol) and potassium vinyltrifluoroborate (222 mg, 1.7 mmol) in 1,4-dioxane (5.0 mL) and water (0.5 mL) were added sodium bicarbonate (146.4 mg, 1.4 mmol) and Pd(dppf)Cl₂·CH₂Cl₂ (45 mg, 55 μmol). The mixture was stirred at 110° C. for 12 h under nitrogen, then concentrated. The residue was purified by flash column chromatography (3-7% methanol in dichloromethane) to afford 3-methyl-2-[3-[3-[(4-methyl-1,2,4-triazol-3-yl)methyl]oxetan-3-yl]phenyl]-4-(trifluoromethyl)-6-vinyl-isoindolin-1-one (258 mg, 96%) as a brown foam. MS (ESI) m/z: 469.1 [M+H]⁺.

### Step 8: Preparation of 1-methyl-2-[3-[3-[(4-methyl-1,2,4-triazol-3-yl)methyl]oxetan-3-yl]phenyl]-3-oxo-7-(trifluoromethyl)isoindoline-5-carbaldehyde

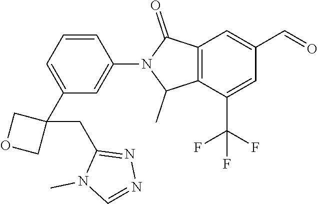 [SMILES: CC1c2c(cc(C=O)cc2C(F)(F)F)C(=O)N1c1cccc(C2(Cc3nncn3C)COC2)c1]

To a solution of 3-methyl-2-[3-[3-[(4-methyl-1,2,4-triazol-3-yl)methyl]oxetan-3-yl]phenyl]-4-(trifluoromethyl)-6-vinyl-isoindolin-1-one (258 mg, 0.5 mmol) in 1,4-dioxane (10 mL) and water (3.0 mL) were added 2,6-dimethylpyridine (123 μL, 1 mmol), potassium osmate(VI) dihydrate (3.9 mg, 11 μmol) and sodium periodate (452 mg, 2.1 mmol). The mixture was stirred at 30° C. for 2 h, then diluted with water (30 mL) and extracted with ethyl dichloromethane (30 mL×3). The combined organic layer was washed with brine (50 mL), dried over sodium sulfate, filtered, and concentrated. The residue was purified by flash column chromatography (3-7% methanol in dichloromethane) to afford 1-methyl-2-[3-[3-[(4-methyl-1,2,4-triazol-3-yl)methyl]oxetan-3-yl]phenyl]-3-oxo-7-(trifluoromethyl)isoindoline-5-carbaldehyde (194 mg, 77%) as a light yellow foam. MS (ESI) m/z: 471.1 [M+H]⁺.

### Step 9: Preparation of tert-butyl (3S)-3-(trifluoromethylsulfonyloxymethyl)piperidine-1-carboxylate

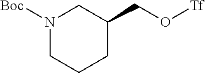

To a solution of tert-butyl (3S)-3-(hydroxymethyl)piperidine-1-carboxylate (3.8 g, 18 mmol) and 2,6-dimethylpyridine (6.17 mL, 53 mmol) in dichloromethane (40 mL) was added triflic anhydride (8.74 mL, 53 mmol) at −78° C. under nitrogen atmosphere. The reaction mixture was stirred at −78° C. for 1 h, then quenched with saturated ammonium chloride solution (25 mL) at −78° C. The mixture was extracted with dichloromethane (30 mL), the organic layer was washed with brine (10 mL), dried over anhydrous sodium sulfate, filtered, and concentrated to afford tert-butyl (3S)-3-(trifluoromethylsulfonyloxymethyl)piperidine-1-carboxylate (6.13 g, crude) as a yellow oil, which was used directly in the next step.

### Step 10: Preparation of 04-benzyl 01-tert-butyl 2,2-dimethylpiperazine-1,4-dicarboxylate

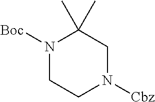 [SMILES: CC(C)(C)OC(=O)N1CCN(C(=O)OCc2ccccc2)CC1(C)C]

To a solution of tert-butyl 2,2-dimethylpiperazine-1-carboxylate (10.0 g, 47 mmol) and sodium bicarbonate (11.76 g, 140 mmol) in water (50 mL) and tetrahydrofuran (150 mL) was added benzyl chloroformate (10 mL, 70 mmol) at 0° C. The mixture was stirred at 20° C. for 12 h, then diluted with water (20 mL) and extracted with ethyl acetate (100 mL×3). The combined organic phase was washed with brine (30 mL), dried over anhydrous sodium sulfate, filtered, and concentrated. The residue was purified by flash column chromatography (0-12% ethyl acetate in petroleum ether) to afford 04-benzyl 01-tert-butyl 2,2-dimethylpiperazine-1,4-dicarboxylate (17.8 g, crude) as colorless oil. MS (ESI) m/z: 249.1 [M-100+H]⁺; ¹H NMR (400 MHz, CDCl₃) δ 7.39-7.33 (m, 5H), 5.17 (s, 2H), 3.72-3.64 (m, 2H), 3.58-3.50 (m, 2H), 3.50-3.42 (m, 2H), 1.48 (s, 9H), 1.42-1.36 (m, 6H).

### Step 11: Preparation of benzyl 3,3-dimethylpiperazine-1-carboxylate

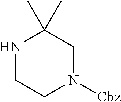 [SMILES: CC1(C)CN(C(=O)OCc2ccccc2)CCN1]

To a solution of 04-benzyl 01-tert-butyl 2,2-dimethylpiperazine-1,4-dicarboxylate (5.0 g, 14 mmol) in dichloromethane (24 mL) was added TFA (8.0 mL), and the mixture was stirred at 25° C. for 30 min. The pH of the reaction mixture was adjusted to 10 by the addition of saturated aqueous sodium bicarbonate solution, and the resulting mixture was extracted with ethyl acetate (30 mL×3). The combined organic phase was washed with brine (40 mL), dried over anhydrous sodium sulfate, filtered, and concentrated to afford benzyl 3,3-dimethylpiperazine-1-carboxylate (3.78 g, 89% purity, 94%) as a yellow oil. MS (ESI) m/z: 249.2 [M+H]⁺.

### Step 12: Preparation of benzyl 4-[[(3R)-1-tert-butoxycarbonyl-3-piperidyl]methyl]-3,3-dimethyl-piperazine-1-carboxylate

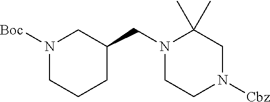 [SMILES: CC(C)(C)OC(=O)N1CCC[C@H](CN2CCN(C(=O)OCc3ccccc3)CC2(C)C)C1]

To a solution of benzyl 3,3-dimethylpiperazine-1-carboxylate (2.88 g, 89% purity, 10 mmol) and N,N-diisopropylethylamine (4.0 g, 31 mmol) in acetonitrile (50 mL) was added tert-butyl (3S)-3-(trifluoromethylsulfonyloxymethyl)piperidine-1-carboxylate (5.38 g, 15 mmol), and the mixture was stirred at 20° C. for 12 h. The reaction mixture was diluted with saturated ammonium chloride solution (40 mL) and extracted with ethyl acetate (30 mL×3). The combined organic phase was washed with brine (30 mL), dried over anhydrous sodium sulfate, filtered, and concentrated. The residue was purified by flash column chromatography (0-12% ethyl acetate in petroleum ether) to afford benzyl 4-[[(3R)-1-tert-butoxycarbonyl-3-piperidyl]methyl]-3,3-dimethyl-piperazine-1-carboxylate (3.49 g, 76%) as a yellow oil. MS (ESI) m/z: 446.7 [M+H]⁺; ¹H NMR (400 MHz, CDCl₃) δ 7.39-7.28 (m, 5H), 5.14 (s, 2H), 4.10-3.95 (m, 1H), 3.90 (br d, J=13.2 Hz, 1H), 3.66-3.35 (m, 2H), 3.32-3.05 (m, 2H), 2.77 (br t, J=11.2 Hz, 1H), 2.59-2.36 (m, 3H), 2.25-2.15 (m, 1H), 2.13-2.06 (m, 1H), 1.79 (br d, J=11.2 Hz, 1H), 1.61 (br t, J=3.6 Hz, 1H), 1.58-1.52 (m, 1H), 1.46 (s, 10H), 1.10-1.01 (m, 1H), 0.99-0.91 (m, 6H).

### Step 13: Preparation of tert-butyl (3R)-3-[(2,2-dimethylpiperazin-1-yl)methyl]piperidine-1-carboxylate

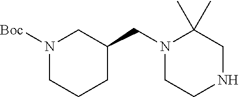 [SMILES: CC(C)(C)OC(=O)N1CCC[C@H](CN2CCNCC2(C)C)C1]

To a solution of benzyl 4-[[(3R)-1-tert-butoxycarbonyl-3-piperidyl]methyl]-3,3-dimethyl-piperazine-1-carboxylate (3.49 g, 8 mmol) in tetrahydrofuran (40 mL) was added 10% palladium on carbon (1.0 g), the resulting suspension was degassed under vacuum and purged with hydrogen several times. The mixture was stirred under hydrogen (15 psi) at 25° C. for 4 h. The reaction mixture was filtered and concentrated under reduced pressure to afford tert-butyl (3R)-3-[(2,2-dimethylpiperazin-1-yl)methyl]piperidine-1-carboxylate (2.42 g, crude) as brown oil. ¹H NMR (400 MHz, CDCl₃) δ 4.17-3.98 (m, 1H), 3.91 (br d, J=13.2 Hz, 1H), 2.86 (br s, 2H), 2.77 (br t, J=11.6 Hz, 1H), 2.57 (d, J=1.6 Hz, 2H), 2.51-2.44 (m, 1H), 2.39 (br dd, J=5.6, 11.6 Hz, 1H), 2.23-2.03 (m, 2H), 1.79 (br s, 1H), 1.73 (br s, 2H), 1.66-1.59 (m, 1H), 1.58-1.50 (m, 1H), 1.46 (s, 9H), 1.10-1.01 (m, 1H), 0.97 (s, 6H).

### Step 14: Preparation of tert-butyl (3R)-3-[[4-[[4-[(1-benzyloxycarbonyl-4-piperidyl)oxy]cyclohexyl]methyl]-2,2-dimethyl-piperazin-1-yl]methyl]piperidine-1-carboxylate

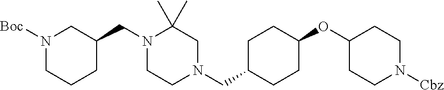 [SMILES: CC(C)(C)OC(=O)N1CCC[C@H](CN2CCN(C[C@H]3CC[C@H](OC4CCN(C(=O)OCc5ccccc5)CC4)CC3)CC2(C)C)C1]

To a solution of tert-butyl (3R)-3-[(2,2-dimethylpiperazin-1-yl)methyl]piperidine-1-carboxylate (2.4 g, 7.7 mmol) and benzyl 4-[4-(p-tolylsulfonyloxymethyl)cyclohexoxy]piperidine-1-carboxylate (3.87 g, 7.7 mmol) in DMSO (20 mL) was added N,N-diisopropylethylamine (4.0 mL, 23 mmol). The mixture was stirred at 120° C. for 12 h, then diluted with water (20 mL) and extracted with ethyl acetate (30 mL×3). The combined organic phase was washed with brine (30 mL), dried over anhydrous sodium sulfate, filtered, and concentrated. The residue was purified by flash column chromatography (0-2% methanol in dichloromethane) to afford tert-butyl (3R)-3-[[4-[[4-[(1-benzyloxycarbonyl-4-piperidyl)oxy]cyclohexyl]methyl]-2,2-dimethyl-piperazin-1-yl]methyl]piperidine-1-carboxylate (4.74 g, 96%) as a yellow oil. MS (ESI) m/z: 641.9 [M+H]⁺; ¹H NMR (400 MHz, CDCl₃) δ 7.37-7.34 (m, 5H), 5.13 (s, 2H), 3.95-3.81 (m, 4H), 3.62-3.56 (m, 1H), 3.27 (br d, J=4.0 Hz, 1H), 3.23-3.12 (m, 3H), 2.76 (br d, J=2.4 Hz, 1H), 2.62 (s, 1H), 2.56-2.41 (m, 3H), 2.22-2.09 (m, 2H), 2.00 (br d, J=7.2 Hz, 3H), 1.94 (br d, J=1.6 Hz, 2H), 1.89-1.73 (m, 7H), 1.56-1.50 (m, 3H), 1.46 (s, 111H), 1.23 (br d, J=12.4 Hz, 2H), 1.00 (br s, 1H), 0.97 (s, 6H), 0.93-0.78 (m, 2H).

### Step 15: Preparation of tert-butyl (3R)-3-[[2,2-dimethyl-4-[[4-(4-piperidyloxy)cyclohexyl]methyl]piperazin-1-yl]methyl]piperidine-1-carboxylate

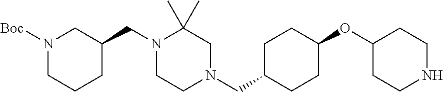 [SMILES: CC(C)(C)OC(=O)N1CCC[C@H](CN2CCN(C[C@H]3CC[C@H](OC4CCNCC4)CC3)CC2(C)C)C1]

To a solution of tert-butyl (3R)-3-[[4-[[4-[(1-benzyloxycarbonyl-4-piperidyl)oxy]cyclohexyl]methyl]-2,2-dimethyl-piperazin-1-yl]methyl]piperidine-1-carboxylate (2.0 g, 3 mmol) in tetrahydrofuran (20 mL) was added 10% palladium on carbon (0.5 g), and the suspension was degassed under vacuum and purged with hydrogen several times. The mixture was stirred under hydrogen (15 psi) at 25° C. for 12 h, then filtered and concentrated to afford tert-butyl (3R)-3-[[2,2-dimethyl-4-[[4-(4-piperidyloxy)cyclohexyl]methyl]piperazin-1-yl]methyl]piperidine-1-carboxylate (1.49 g, crude) as brown oil. MS (ESI) m/z: 507.4 [M+H]⁺.

### Step 16: Preparation of tert-butyl (3R)-3-[[4-[[4-[[1-[2-(2,6-dioxo-3-piperidyl)-4-fluoro-1-oxo-isoindolin-5-yl]-4-piperidyl]oxy]cyclohexyl]methyl]-2,2-dimethyl-piperazin-1-yl]methyl]piperidine-1-carboxylate

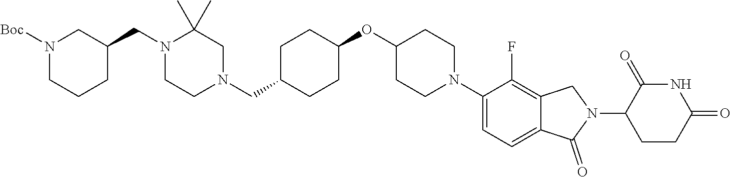 [SMILES: CC(C)(C)OC(=O)N1CCC[C@H](CN2CCN(C[C@H]3CC[C@H](OC4CCN(c5ccc6c(c5F)CN(C5CCC(=O)NC5=O)C6=O)CC4)CC3)CC2(C)C)C1]

To a solution of tert-butyl (3R)-3-[[2,2-dimethyl-4-[[4-(4-piperidyloxy)cyclohexyl]methyl]piperazin-1-yl]methyl]piperidine-1-carboxylate (1.49 g, 2 mmol) and 3-(4,5-difluoro-1-oxo-isoindolin-2-yl)piperidine-2,6-dione (305 mg, 1 mmol) in DMSO (10 mL) was added N,N-diisopropylethylamine (568 μL, 3 mmol). The mixture was stirred at 140° C. for 12 h, then filtered and concentrated. The residue was purified by prep-HPLC (5%-45% acetonitrile in water (0.225% formic acid) over 20 min at 60 mL/min) to afford tert-butyl (3R)-3-[[4-[[4-[[1-[2-(2,6-dioxo-3-piperidyl)-4-fluoro-1-oxo-isoindolin-5-yl]-4-piperidyl]oxy]cyclohexyl]methyl]-2,2-dimethyl-piperazin-1-yl]methyl]piperidine-1-carboxylate (193 mg, 87% purity, 20%) as a yellow solid. MS (ESI) m/z: 767.5 [M+H]⁺.

### Step 17: Preparation of 3-[5-[4-[4-[[3,3-dimethyl-4-[[(3S)-3-piperidyl]methyl]piperazin-1-yl]methyl]cyclohexoxy]-1-piperidyl]-4-fluoro-1-oxo-isoindolin-2-yl]piperidine-2,6-dione

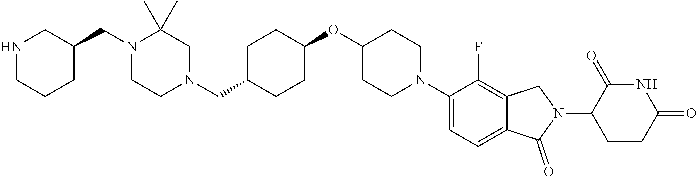 [SMILES: CC1(C)CN(C[C@H]2CC[C@H](OC3CCN(c4ccc5c(c4F)CN(C4CCC(=O)NC4=O)C5=O)CC3)CC2)CCN1C[C@H]1CCCNC1]

To a solution of tert-butyl (3R)-3-[[4-[[4-[[1-[2-(2,6-dioxo-3-piperidyl)-4-fluoro-1-oxo-isoindolin-5-yl]-4-piperidyl]oxy]cyclohexyl]methyl]-2,2-dimethyl-piperazin-1-yl]methyl]piperidine-1-carboxylate (193 mg, 87% purity, 0.2 mmol) in dichloromethane (5.0 mL) was added TFA (1.0 mL). The reaction solution was stirred at 25° C. for 30 min, then concentrated under reduced pressure to afford 3-[5-[4-[4-[[3,3-dimethyl-4-[[(3S)-3-piperidyl]methyl]piperazin-1-yl]methyl]cyclohexoxy]-1-piperidyl]-4-fluoro-1-oxo-isoindolin-2-yl]piperidine-2,6-dione (421 mg, crude, 11 TFA salt) as a yellow oil.

### Step 18: Preparation of 3-(5-(4-(((1S,4r)-4-((3,3-dimethyl-4-(((3S)-1-((1-methyl-2-(3-(3-((4-methyl-4H-1,2,4-triazol-3-yl)methyl)oxetan-3-yl)phenyl)-3-oxo-7-(trifluoromethyl)isoindolin-5-yl)methyl)piperidin-3-yl)methyl)piperazin-1-yl)methyl)cyclohexyl)oxy)piperidin-1-yl)-4-fluoro-1-oxoisoindolin-2-yl)piperidine-2,6-dione

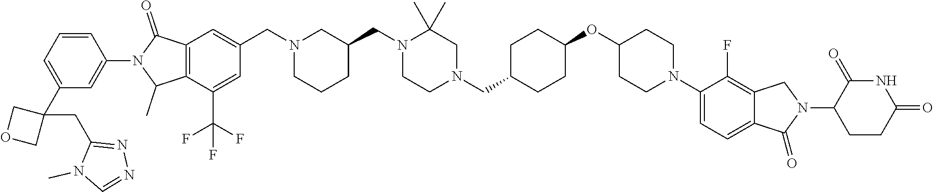 [SMILES: CC1c2c(cc(CN3CCC[C@H](CN4CCN(C[C@H]5CC[C@H](OC6CCN(c7ccc8c(c7F)CN(C7CCC(=O)NC7=O)C8=O)CC6)CC5)CC4(C)C)C3)cc2C(F)(F)F)C(=O)N1c1cccc(C2(Cc3nncn3C)COC2)c1]

To a solution of 3-[5-[4-[4-[[3,3-dimethyl-4-[[(3S)-3-piperidyl]methyl]piperazin-1-yl]methyl]cyclohexoxy]-1-piperidyl]-4-fluoro-1-oxo-isoindolin-2-yl]piperidine-2,6-dione (421 mg, 219 μmol, 11 TFA) and 1-methyl-2-[3-[3-[(4-methyl-1,2,4-triazol-3-yl)methyl]oxetan-3-yl]phenyl]-3-oxo-7-(trifluoromethyl)isoindoline-5-carbaldehyde (139 mg, 260 μmol) in 1,2-dichloroethane (5.0 mL) and DMSO (1.0 mL) were added chloro(triisopropoxy)titanium (85 mg, 0.3 mmol), sodium acetate (269 mg, 3 mmol) and molecular sieves (1.0 g). The mixture was stirred at 25° C. for 12 h under nitrogen before the addition of NaBH(OAc)₃ (139 mg, 657 μmol). The reaction mixture was stirred 25° C. for 1 h, then concentrated. The residue was purified by prep-HPLC (0-34% acetonitrile in water (0.225% formic acid) over 36 min at 60 mL/min), and the crude product was further purified by prep-HPLC (58-96% acetonitrile in water (0.225% NH₄HCO₃) over 32 min at 60 mL/min) to afford 3-(5-(4-(((1S,4r)-4-((3,3-dimethyl-4-(((3S)-1-((1-methyl-2-(3-(3-((4-methyl-4H-1,2,4-triazol-3-yl)methyl)oxetan-3-yl)phenyl)-3-oxo-7-(trifluoromethyl)isoindolin-5-yl)methyl)piperidin-3-yl)methyl)piperazin-1-yl)methyl)cyclohexyl)oxy)piperidin-1-yl)-4-fluoro-1-oxoisoindolin-2-yl)piperidine-2,6-dione (56.8 mg, 55%) as a white solid. MS (ESI) m/z: 1121.7 [M+H]⁺; ¹H NMR (400 MHz, DMSO-d₆) δ 11.06 (s, 1H), 8.17 (s, 1H), 7.98 (s, 1H), 7.92 (s, 1H), 7.87-7.83 (m, 1H), 7.45 (s, 1H), 7.34 (t, J=8.0 Hz, 1H), 6.92-6.88 (m, 2H), 6.82 (d, J=7.6 Hz, 1H), 6.61 (dd, J=8.4, 1.6 Hz, 1H), 5.26 (dd, J=12.8, 5.2 Hz, 1H), 5.10 (s, 2H), 4.62-4.54 (m, 1H), 4.10 (d, J=14.0 Hz, 1H), 3.56-3.50 (m, 1H), 3.39 (d, J=7.2 Hz, 3H), 3.30 (s, 3H), 3.28-3.17 (m, 2H), 2.87 (d, J=7.2 Hz, 5H), 2.77 (s, 4H), 2.65-2.55 (m, 4H), 2.17 (t, J=8.8 Hz, 1H), 2.06-1.96 (m, 4H), 1.90 (t, J=11.6 Hz, 6H), 1.78-1.72 (m, 2H), 1.53 (d, J=9.2 Hz, 2H), 1.43 (d, J=6.8 Hz, 8H), 1.15-1.10 (m, 2H), 1.08 (d, J=6.0 Hz, 3H), 0.89-0.80 (m, 2H).

### Example 277: Exemplary synthesis of (1r, 3r)-3-(3-{6-[(4-{[4-({1-[6-(2,6-dioxopiperidin-3-yl)-7-methyl-5-oxo-5H, 6H, 7H-pyrrolo [3,4-b]pyridin-2-yl]piperidin-4-yl}methyl)piperazin-1-yl]methyl}-2,2-dimethylpiperidin-1-yl)methyl]-1-oxo-4-(trifluoromethyl)-2,3-dihydro-1H-isoindol-2-yl}phenyl)-3-[(4-methyl-4H-1,2,4-triazol-3-yl)methyl]cyclobutane-1-carbonitrile Step 1: Preparation of tert-butyl 4-[[1-[6-(2,6-dioxo-3-piperidyl)-7-methyl-5-oxo-7H-pyrrolo[3,4-b]pyridin-2-yl]-4-piperidyl]methyl]piperazine-1-carboxylate

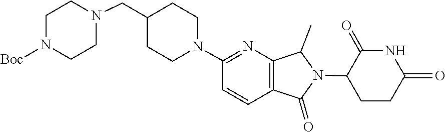 [SMILES: CC1c2nc(N3CCC(CN4CCN(C(=O)OC(C)(C)C)CC4)CC3)ccc2C(=O)N1C1CCC(=O)NC1=O]

To a solution of 1-[6-(2,6-dioxo-3-piperidyl)-7-methyl-5-oxo-7H-pyrrolo[3,4-b]pyridin-2-yl]piperidine-4-carbaldehyde trifluoroacetate (462 mg, 0.95 mmol) in dichloromethane (5 mL) were added tert-butyl piperazine-1-carboxylate (213 mg, 1.1 mmol) and triethylamine (290 mg, 2.9 mmol), and the mixture was stirred at 25° C. for 8 h before the addition of sodium triacetoxyborohydride (404 mg, 1.9 mmol). The resulting mixture was stirred at 25° C. for 2 h, then concentrated under reduced pressure. The residue was purified by silica gel column chromatography (dichloromethane/methanol=30/1 to 10/1) to afford tert-butyl 4-[[1-[6-(2,6-dioxo-3-piperidyl)-7-methyl-5-oxo-7H-pyrrolo[3,4-b]pyridin-2-yl]-4-piperidyl]methyl]piperazine-1-carboxylate (500 mg, 97%) as a white solid. ¹H NMR (400 MHz, DMSO-d₆) δ 10.88 (d, J=12.4 Hz, 1H), 7.76-7.62 (m, 1H), 6.95-6.79 (m, 1H), 6.98-6.78 (m, 1H), 4.77-4.64 (m, 1H), 4.56-4.36 (m, 3H), 4.04 (q, J=7.2 Hz, 1H), 3.16-3.06 (m, 1H), 3.00-2.90 (m, 2H), 2.86-2.58 (m, 3H), 2.38-2.26 (m, 3H), 2.15 (d, J=4.0 Hz, 2H), 2.02-1.97 (m, 2H), 1.92-1.71 (m, 3H), 1.45-1.33 (m, 12H), 1.18 (t, J=7.2 Hz, 2H), 1.16-1.01 (m, 2H).

### Step 2: Preparation of 3-[7-methyl-5-oxo-2-[4-(piperazin-1-ylmethyl)-1-piperidyl]-7H-pyrrolo[3,4-b]pyridin-6-yl]piperidine-2,6-dione

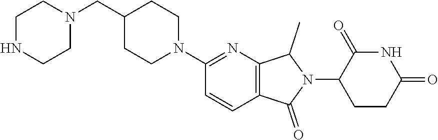 [SMILES: CC1c2nc(N3CCC(CN4CCNCC4)CC3)ccc2C(=O)N1C1CCC(=O)NC1=O]

To a solution of tert-butyl 4-[[1-[6-(2,6-dioxo-3-piperidyl)-7-methyl-5-oxo-7H-pyrrolo[3,4-b]pyridin-2-yl]-4-piperidyl]methyl]piperazine-1-carboxylate (80 mg, 0.2 mmol) in dichloromethane (1 mL) was added trifluoroacetic acid (460 mg, 4 mmol). The reaction solution was stirred at 25° C. for 0.2 h, then concentrated under reduced pressure to afford 3-[7-methyl-5-oxo-2-[4-(piperazin-1-ylmethyl)-1-piperidyl]-7H-pyrrolo[3,4-b]pyridin-6-yl]piperidine-2,6-dione trifluoroacetate (82 mg, crude, TFA salt) as a yellow oil. MS (ESI) m/z: 441.3 [M+H]⁺.

### Step 3: Preparation of 2,2-dimethylpiperidin-4-one

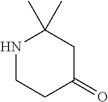

To a solution of tert-butyl 2,2-dimethyl-4-oxo-piperidine-1-carboxylate (3.80 g, 17 mmol) in dichloromethane (15 mL) was added trifluoroacetic acid (7.29 g, 64 mmol). The mixture was stirred at 25° C. for 0.5 h, then concentrated under reduced pressure to afford 2,2-dimethylpiperidin-4-one trifluoroacetate (4.00 g, crude, TFA salt) as a colorless oil.

### Step 4: Preparation of 2,6-dibenzyloxy-N-[3-[4-(dimethoxymethyl)-1-piperidyl]-2-nitro-phenyl]pyridin-3-amine

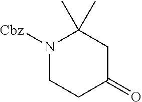 [SMILES: CC1(C)CC(=O)CCN1C(=O)OCc1ccccc1]

To a solution of 2,2-dimethylpiperidin-4-one trifluoroacetate (4.00 g, 16 mmol) in dichloromethane (40 mL) were added benzyl carbonochloridate (3.39 g, 20 mmol) and triethylamine (3.36 g, 33 mmol) at 0° C. The mixture was stirred at 25° C. for 10 h. The reaction mixture was concentrated under reduced pressure, and the residue was purified by silica gel column chromatography (petroleum ether/ethyl acetate=20/1 to 10/1) to afford benzyl 2,2-dimethyl-4-oxo-piperidine-1-carboxylate (1.00 g, 23%) as a colorless oil. ¹H NMR (400 MHz, DMSO-d₆) δ 7.40-7.32 (m, 5H), 5.09 (s, 2H), 3.90 (t, J=6.0 Hz, 2H), 2.65 (s, 2H), 2.40 (t, J=6.0 Hz, 2H), 1.39 (s, 6H).

### Step 5: Preparation of benzyl (4Z)-4-(methoxymethylene)-2,2-dimethyl-piperidine-1-carboxylate

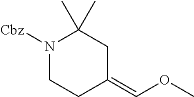 [SMILES: CO/C=C1/CCN(C(=O)OCc2ccccc2)C(C)(C)C1]

To a solution of methoxymethyl(triphenyl)phosphonium chloride (1.67 g, 4.9 mmol) in tetrahydrofuran (10 mL) was added potassium tert-butoxide (1 M, 6.5 mL) at 0° C. The mixture was stirred at 0° C. for 1 h before the addition of benzyl 2,2-dimethyl-4-oxo-piperidine-1-carboxylate (850 mg, 3.3 mmol). The resulting mixture was stirred at 25° C. for 10 h. The reaction mixture was diluted with saturated aqueous ammonium chloride solution (50 mL) and extracted with ethyl acetate (50 mL×2). The combined organic layers were concentrated under reduced pressure. The residue was purified by silica gel column chromatography (petroleum ether/ethyl acetate=30/1 to 20/1) to afford benzyl (4Z)-4-(methoxymethylene)-2,2-dimethyl-piperidine-1-carboxylate (750 mg, 79%) as a colorless oil. ¹H NMR (400 MHz, DMSO-d₆) δ 7.40-7.29 (m, 5H), 6.07-5.94 (m, 1H), 5.04 (d, J=1.6 Hz, 2H), 3.58-3.52 (m, 2H), 3.50 (d, J=13.2 Hz, 3H), 2.30-2.13 (m, 4H), 1.33 (d, J=5.2 Hz, 6H).

### Step 6: Preparation of benzyl 4-formyl-2,2-dimethyl-piperidine-1-carboxylate

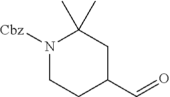 [SMILES: CC1(C)CC(C=O)CCN1C(=O)OCc1ccccc1]

To a solution of benzyl (4Z)-4-(methoxymethylene)-2,2-dimethyl-piperidine-1-carboxylate (650 mg, 2.3 mmol) in water (0.7 mL) and acetone (7 mL) was added 10 M hydrochloric acid (0.12 mL). The mixture was stirred at 50° C. for 10 h, then concentrated under reduced pressure. The residue was purified by silica gel column chromatography (petroleum ether/ethyl acetate=30/1 to 20/1) to afford benzyl 4-formyl-2,2-dimethyl-piperidine-1-carboxylate (456 mg, 74%) as a colorless oil. ¹H NMR (400 MHz, DMSO-d₆) δ 9.61 (s, 1H), 7.43-7.28 (m, 5H), 5.04 (s, 2H), 3.41-3.67 (m, 1H), 3.43-3.38 (m, 1H), 2.76-2.64 (m, 1H), 1.90-1.74 (m, 2H), 1.72-1.59 (m, 2H), 1.47-1.31 (m, 6H).

### Step 7: Preparation of 1-(2,6-dibenzyloxy-3-pyridyl)-4-[4-(dimethoxymethyl)-1-piperidyl]-3-isopropyl-benzimidazol-2-one

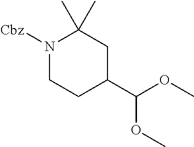 [SMILES: COC(OC)C1CCN(C(=O)OCc2ccccc2)C(C)(C)C1]

To a solution of benzyl 4-formyl-2,2-dimethyl-piperidine-1-carboxylate (456 mg, 1.7 mmol) and trimethoxymethane (1.76 g, 17 mmol) in methanol (5 mL) was added p-toluene sulfonic acid (86 mg, 0.5 mmol). The mixture was stirred at 25° C. for 10 h, then diluted with water (50 mL) and extracted with ethyl acetate (50 mL×2). The combined organic layers were concentrated under reduced pressure to afford benzyl 4-(dimethoxymethyl)-2,2-dimethyl-piperidine-1-carboxylate (325 mg, 61%) as a colorless oil. ¹H NMR (400 MHz, DMSO-d₆) δ 7.39-7.33 (m, 5H), 5.03 (s, 2H), 4.07 (d, J=7.2 Hz, 1H), 3.63-3.58 (m, 1H), 3.25 (s, 6H), 1.99-1.89 (m, 1H), 1.68 (J=6.0, 11.6 Hz, 1H), 1.51-1.41 (m, 5H), 1.37-1.19 (m, 5H).

### Step 8: Preparation of 1-(2,6-dibenzyloxy-3-pyridyl)-4-[4-(dimethoxymethyl)-1-piperidyl]-3-isopropyl-benzimidazol-2-one

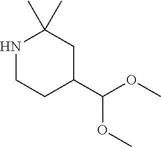

To a solution of benzyl 4-(dimethoxymethyl)-2,2-dimethyl-piperidine-1-carboxylate (307 mg, 0.96 mmol) in methanol (5 mL) was added 10% palladium on carbon (100 mg) under nitrogen atmosphere. The suspension was degassed under vacuum and purged with hydrogen several times. The mixture was stirred under hydrogen (15 psi) at 25° C. for 10 h, then filtered and concentrated under reduced pressure to afford 4-(dimethoxymethyl)-2,2-dimethyl-piperidine (150 mg, 84%) as a colorless oil. ¹H NMR (400 MHz, DMSO-d₆) δ 3.93 (d, J=7.2 Hz, 1H), 3.23 (d, J=4.4 Hz, 6H), 2.74-2.60 (m, 2H), 1.88-1.74 (m, 1H), 1.52 (d, J=12.4 Hz, 1H), 1.46-1.38 (m, 1H), 1.01 (s, 6H), 0.95-0.83 (m, 2H).

### Step 9: Preparation of 3-[3-[6-[[4-(dimethoxymethyl)-2,2-dimethyl-1-piperidyl]methyl]-1-oxo-4-(trifluoromethyl)isoindolin-2-yl]phenyl]-3-[(4-methyl-1,2,4-triazol-3-yl)methyl]cyclobutanecarbonitrile

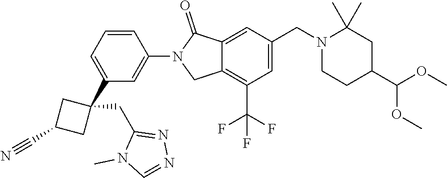 [SMILES: COC(OC)C1CCN(Cc2cc3c(c(C(F)(F)F)c2)CN(c2cccc([C@]4(Cc5nncn5C)C[C@@H](C#N)C4)c2)C3=O)C(C)(C)C1]

To a solution of 3-[3-[6-(chloromethyl)-1-oxo-4-(trifluoromethyl)isoindolin-2-yl]phenyl]-3-[(4-methyl-1,2,4-triazol-3-yl)methyl]cyclobutanecarbonitrile (180 mg, 0.36 mmol) and 4-(dimethoxymethyl)-2,2-dimethyl-piperidine (135 mg, 0.72 mmol) in acetonitrile (0.5 mL) were added triethylamine (11 mg, 0.11 mmol) and potassium iodide (299 mg, 1.8 mmol). The mixture was stirred at 60° C. for 10 h, then filtered and concentrated under reduced pressure. The residue was purified by prep-HPLC (15%-45% acetonitrile in water (formic acid) over 10 min) to afford 3-[3-[6-[[4-(dimethoxymethyl)-2,2-dimethyl-1-piperidyl]methyl]-1-oxo-4-(trifluoromethyl)isoindolin-2-yl]phenyl]-3-[(4-methyl-1,2,4-triazol-3-yl)methyl]cyclobutanecarbonitrile (80 mg, 34%) as a white solid. MS (ESI) m/z: 652.3 [M+H]⁺.

### Step 10: Preparation of 3-[3-[6-[(4-formyl-2,2-dimethyl-1-piperidyl)methyl]-1-oxo-4-(trifluoromethyl)isoindolin-2-yl]phenyl]-3-[(4-methyl-1,2,4-triazol-3-yl)methyl]cyclobutanecarbonitrile

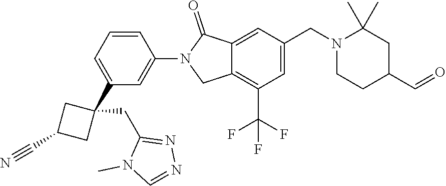 [SMILES: Cn1cnnc1C[C@]1(c2cccc(N3Cc4c(cc(CN5CCC(C=O)CC5(C)C)cc4C(F)(F)F)C3=O)c2)C[C@H](C#N)C1]

To a solution of 3-[3-[6-[[4-(dimethoxymethyl)-2,2-dimethyl-1-piperidyl]methyl]-1-oxo-4-(trifluoromethyl)isoindolin-2-yl]phenyl]-3-[(4-methyl-1,2,4-triazol-3-yl)methyl]cyclobutanecarbonitrile (80 mg, 0.1 mmol) in dichloromethane (1 mL) was added trifluoroacetic acid (461 mg, 4 mmol). The reaction solution was stirred at 25° C. for 0.5 h, then concentrated under reduced pressure to afford 3-[3-[6-[(4-formyl-2,2-dimethyl-1-piperidyl)methyl]-1-oxo-4-(trifluoromethyl)isoindolin-2-yl]phenyl]-3-[(4-methyl-1,2,4-triazol-3-yl)methyl]cyclobutanecarbonitrile (74 mg, crude, TFA salt) as a yellow oil. MS (ESI) m/z: 605.4 [M+H]⁺.

### Step 11: Preparation of 3-[3-[6-[[4-[[4-[[1-[6-(2,6-dioxo-3-piperidyl)-7-methyl-5-oxo-7H-pyrrolo[3,4-b]pyridin-2-yl]-4-piperidyl]methyl]piperazin-1-yl]methyl]-2,2-dimethyl-1-piperidyl]methyl]-1-oxo-4-(trifluoromethyl)isoindolin-2-yl]phenyl]-3-[(4-methyl-1,2,4-triazol-3-yl)methyl]cyclobutanecarbonitrile

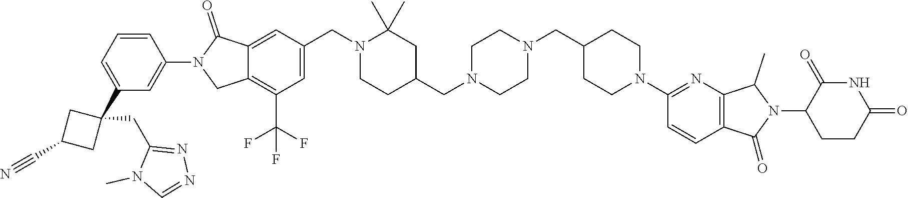 [SMILES: CC1c2nc(N3CCC(CN4CCN(CC5CCN(Cc6cc7c(c(C(F)(F)F)c6)CN(c6cccc([C@]8(Cc9nncn9C)C[C@@H](C#N)C8)c6)C7=O)C(C)(C)C5)CC4)CC3)ccc2C(=O)N1C1CCC(=O)NC1=O]

To a solution of 3-[3-[6-[(4-formyl-2,2-dimethyl-1-piperidyl)methyl]-1-oxo-4-(trifluoromethyl)isoindolin-2-yl]phenyl]-3-[(4-methyl-1,2,4-triazol-3-yl)methyl]cyclobutanecarbonitrile (74 mg, 0.1 mmol) and 3-[7-methyl-5-oxo-2-[4-(piperazin-1-ylmethyl)-1-piperidyl]-7H-pyrrolo[3,4-b]pyridin-6-yl]piperidine-2,6-dione (65 mg, 0.2 mmol) in dichloromethane (1 mL) was added triethylamine (37 mg, 0.4 mmol). The mixture was stirred at 0° C. for 0.5 h before the addition of sodium triacetoxyborohydride (52 mg, 0.2 mmol). The reaction mixture was stirred at 25° C. for 1.5 h, then concentrated under reduced pressure. The residue was purified by prep-HPLC (10%-40% acetonitrile in water (formic acid) over 10 min) to afford 3-[3-[6-[[4-[[4-[[1-[6-(2,6-dioxo-3-piperidyl)-7-methyl-5-oxo-7H-pyrrolo[3,4-b]pyridin-2-yl]-4-piperidyl]methyl]piperazin-1-yl]methyl]-2,2-dimethyl-1-piperidyl]methyl]-1-oxo-4-(trifluoromethyl)isoindolin-2-yl]phenyl]-3-[(4-methyl-1,2,4-triazol-3-yl)methyl]cyclobutanecarbonitrile (42.7 mg, 30%, formic acid salt) as a white solid. MS (ESI) m/z: 1029.8 [M+H]⁺; ¹H NMR (400 MHz, DMSO-d₆) δ 10.88 (d, J=12.4 Hz, 1H), 8.17 (s, 1H), 8.15-8.12 (m, 1H), 8.10-7.88 (m, 2H), 7.84 (d, J=7.6 Hz, 1H), 7.70 (J=2.4, 8.8 Hz, 1H), 7.46 (s, 1H), 7.39-7.33 (m, 1H), 6.87 (t, J=5.6 Hz, 2H), 5.10 (s, 2H), 4.75-4.64 (m, 1H), 4.51-4.38 (m, 3H), 4.27-4.10 (m, 1H), 3.25-3.17 (m, 4H), 3.03-2.91 (m, 4H), 2.89 (s, 2H), 2.87 (d, J=2.0 Hz, 2H), 2.84 (s, 1H), 2.80 (s, 4H), 2.78 (s, 1H), 2.60 (d, J=6.8 Hz, 3H), 2.45-2.35 (m, 4H), 2.08-1.84 (m, 4H), 1.83-1.72 (m, 3H), 1.72-1.48 (m, 3H), 1.39 (J=6.8, 14.0 Hz, 4H), 1.32-1.21 (m, 3H), 1.21-1.14 (m, 2H), 1.13 d, J=3.2 Hz, 6H).

### Example 278: Exemplary synthesis of (1r, 3r)-3-(3-{6-[7-(2-{1-[2-(2,6-dioxopiperidin-3-yl)-4-fluoro-7-methyl-1-oxo-2,3-dihydro-1H-isoindol-5-yl]piperidin-4-yl}propan-2-yl)-2,7-diazaspiro[3.5]nonan-2-yl]-1-oxo-4-(trifluoromethyl)-2,3-dihydro-1H-isoindol-2-yl}phenyl)-3-[(4-methyl-4H-1,2,4-triazol-3-yl)methyl]cyclobutane-1-carbonitrile

### Step 1: Preparation of tert-butyl 7-(1-((benzyloxy)carbonyl)piperidine-4-carbonyl)-2,7-diazaspiro[3.5]nonane-2-carboxylate

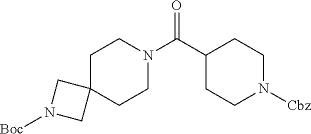 [SMILES: CC(C)(C)OC(=O)N1CC2(CCN(C(=O)C3CCN(C(=O)OCc4ccccc4)CC3)CC2)C1]

To a solution of tert-butyl 2,7-diazaspiro[3.5]nonane-2-carboxylate (3.44 g, 15 mmol) and 1-benzyloxycarbonylpiperidine-4-carboxylic acid (4.40 g, 17 mmol) in dimethyl formamide (40 mL) were added N,N-diisopropylethylamine (5.3 mL, 30 mmol) and 1-[bis(dimethylamino)methylene]-1H-1,2,3-triazolo[4,5-b]pyridinium 3-oxid hexafluorophosphate (6.93 g, 18 mmol), and the mixture was stirred at 25° C. for 2 h. The reaction mixture was poured into water (400 mL), and the resulting suspension was filtered. The filter cake was dissolved in dichloromethane (50 mL) and dried over anhydrous sulfate sodium. The mixture was filtered and concentrated. The crude product was triturated with petroleum ether/ethyl acetate (50 mL/5 mL) to afford tert-butyl 7-(1-((benzyloxy)carbonyl)piperidine-4-carbonyl)-2,7-diazaspiro[3.5]nonane-2-carboxylate (7 g, 97%) as a white solid. MS (ESI) m/z: 494.3 [M+Na]⁺; ¹H NMR (400 MHz, DMSO-d₆) δ 7.45-7.20 (m, 5H), 5.07 (s, 2H), 3.99 (d, J=13.2 Hz, 2H), 3.57 (s, 4H), 3.41 (s, 3H), 2.99-2.77 (m, 3H), 1.75-1.49 (m, 6H), 1.47-1.40 (m, 2H), 1.38 (s, 9H), 1.26-1.23 (m, 1H).

### Step 2: Preparation of tert-butyl 7-(2-(1-((benzyloxy)carbonyl)piperidin-4-yl)propan-2-yl)-2,7-diazaspiro[3.5]nonane-2-carboxylate

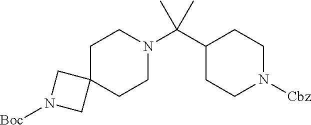 [SMILES: CC(C)(C)OC(=O)N1CC2(CCN(C(C)(C)C3CCN(C(=O)OCc4ccccc4)CC3)CC2)C1]

To a stirred mixture of zirconium tetrachloride (5.93 g, 25 mmol) in tetrahydrofuran (60 mL) were added tert-butyl 7-(1-benzyloxycarbonylpiperidine-4-carbonyl)-2,7-diazaspiro[3.5]nonane-2-carboxylate (6.00 g, 13 mmol) in tetrahydrofuran (10 mL) and 3 M methyl magnesium bromide (25 mL) at −78° C. The reaction mixture was stirred at 25° C. for 12 h, then diluted with saturated aqueous ammonium chloride solution (100 mL) and extracted with ethyl acetate (50 mL×3). The combined organic layers were dried over anhydrous sodium sulfate, filtered, and concentrated under reduced pressure. The residue was purified by column chromatography (petroleum ether/ethyl acetate=20/1 to 1/1) to afford tert-butyl 7-(2-(1-((benzyloxy)carbonyl)piperidin-4-yl)propan-2-yl)-2,7-diazaspiro[3.5]nonane-2-carboxylate (2.90 g, 42%) as a yellow oil. MS (ESI) m/z: 486.4 [M+H]⁺; ¹H NMR (400 MHz, CDCl₃) δ 7.25-7.23 (m, 5H), 5.10-5.08 (m, 2H), 4.19-4.16 (m, 2H), 3.51 (s, 4H), 2.87-2.85 (m, 2H), 2.46-2.44 (m, 2H), 1.85-1.82 (m, 4H), 1.64-1.59 (m, 5H), 1.54 (s, 2H), 1.38 (s, 9H), 0.79 (s, 6H).

### Step 3: Preparation of tert-butyl 7-(2-(piperidin-4-yl)propan-2-yl)-2,7-diazaspiro[3.5]nonane-2-carboxylate

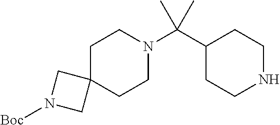 [SMILES: CC(C)(C)OC(=O)N1CC2(CCN(C(C)(C)C3CCNCC3)CC2)C1]

A mixture of tert-butyl 7-(2-(1-((benzyloxy)carbonyl)piperidin-4-yl)propan-2-yl)-2,7-diazaspiro[3.5]nonane-2-carboxylate (2.9 g, 6 mmol) and 10% palladium on carbon (635 mg) in methanol (50 mL) was degassed under vacuum and purged with hydrogen several times, and the mixture was stirred at 25° C. for 10 h under hydrogen atmosphere. The reaction mixture was filtered, and the filtrate was concentrated. The residue was purified by prep-HPLC (5%-35% acetonitrile in water (formic acid) over 15 min), then further purified by prep-HPLC (1%-20% acetonitrile in water (formic acid) over 9 min) to afford tert-butyl 7-(2-(piperidin-4-yl)propan-2-yl)-2,7-diazaspiro[3.5]nonane-2-carboxylate (1.0 g, 42%, formic acid salt) as a white solid. MS (ESI) m/z: 352.3 [M+H]⁺; ¹H NMR (400 MHz, CDCl₃) δ 8.39 (s, 2H), 3.59 (s, 4H), 3.45 (d, J=12.4 Hz, 2H), 2.92-2.73 (m, 2H), 2.51 (s, 4H), 1.93 (d, J=13.6 Hz, 2H), 1.86-1.69 (m, 5H), 1.65-1.49 (m, 2H), 1.44 (s, 9H), 1.03-0.88 (m, 6H).

### Step 4: Preparation of tert-butyl 7-(2-(1-(2-(2,6-dioxopiperidin-3-yl)-4-fluoro-7-methyl-1-oxoisoindolin-5-yl)piperidin-4-yl)propan-2-yl)-2,7-diazaspiro[3.5]nonane-2-carboxylate

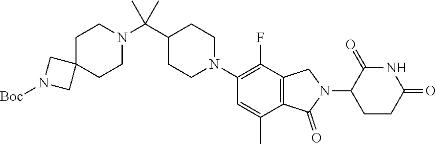 [SMILES: Cc1cc(N2CCC(C(C)(C)N3CCC4(CC3)CN(C(=O)OC(C)(C)C)C4)CC2)c(F)c2c1C(=O)N(C1CCC(=O)NC1=O)C2]

A mixture of tert-butyl 7-(2-(piperidin-4-yl)propan-2-yl)-2,7-diazaspiro[3.5]nonane-2-carboxylate formate (450 mg, 1.1 mmol), 3-(5-bromo-4-fluoro-7-methyl-1-oxo-isoindolin-2-yl)piperidine-2,6-dione (804 mg, 2.3 mmol), cesium carbonate (1.11 g, 3.4 mmol) and XPhos Pd G4 (110 mg, 0.11 mmol) in 1,4-dioxane (20 mL) was degassed and purged with nitrogen. The mixture was stirred at 100° C. for 10 h under nitrogen atmosphere. The reaction mixture was diluted with tetrahydrofuran/ethyl acetate (V/V=50 mL/50 mL) and washed with water (100 mL) then brine (100 mL). The organic layer was dried over anhydrous sodium sulfate, filtered, and concentrated under reduced pressure. The residue was purified by column chromatography (dichloromethane/methanol=1/0 to 10/1), then further purified by prep-TLC (dichloromethane/methanol=10:1) to afford tert-butyl 7-(2-(1-(2-(2,6-dioxopiperidin-3-yl)-4-fluoro-7-methyl-1-oxoisoindolin-5-yl)piperidin-4-yl)propan-2-yl)-2,7-diazaspiro[3.5]nonane-2-carboxylate (800 mg, 45%) as a white solid. MS (ESI) m/z: 626.4 [M+H]⁺.

### Step 5: Preparation of 3-(5-(4-(2-(2,7-diazaspiro[3.5]nonan-7-yl)propan-2-yl)piperidin-1-yl)-4-fluoro-7-methyl-1-oxoisoindolin-2-yl)piperidine-2,6-dione

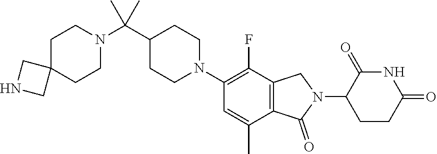 [SMILES: Cc1cc(N2CCC(C(C)(C)N3CCC4(CC3)CNC4)CC2)c(F)c2c1C(=O)N(C1CCC(=O)NC1=O)C2]

To a solution of tert-butyl 7-(2-(1-(2-(2,6-dioxopiperidin-3-yl)-4-fluoro-7-methyl-1-oxoisoindolin-5-yl)piperidin-4-yl)propan-2-yl)-2,7-diazaspiro[3.5]nonane-2-carboxylate (600 mg, 0.96 mmol) in dichloromethane (2 mL) was added trifluoroacetic acid (3.07 g, 27 mmol). The reaction solution was stirred at 25° C. for 1 h, then concentrated under reduced pressure. The residue was purified by prep-HPLC (2%-32% acetonitrile in water (formic acid) over 7 min) to afford 3-[5-[4-[1-(2,7-diazaspiro[3.5]nonan-7-yl)-1-methyl-ethyl]-1-piperidyl]-4-fluoro-7-methyl-1-oxo-isoindolin-2-yl]piperidine-2,6-dione (280 mg, 42%, formic acid salt) as a yellow solid. MS (ESI) m/z: 526.3 [M+H]⁺.

### Step 6: Preparation of (1r, 3r)-3-(3-(6-(7-(2-(1-(2-(2,6-dioxopiperidin-3-yl)-4-fluoro-7-methyl-1-oxoisoindolin-5-yl)piperidin-4-yl)propan-2-yl)-2,7-diazaspiro[3.5]nonan-2-yl)-1-oxo-4-(trifluoromethyl)isoindolin-2-yl)phenyl)-3-((4-methyl-4H-1,2,4-triazol-3-yl)methyl)cyclobutane-1-carbonitrile

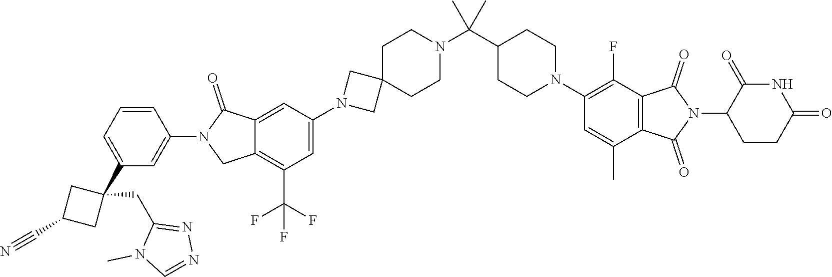 [SMILES: Cc1cc(N2CCC(C(C)(C)N3CCC4(CC3)CN(c3cc5c(c(C(F)(F)F)c3)CN(c3cccc([C@]6(Cc7nncn7C)C[C@@H](C#N)C6)c3)C5=O)C4)CC2)c(F)c2c1C(=O)N(C1CCC(=O)NC1=O)C2=O]

To a solution of 3-[5-[4-[1-(2,7-diazaspiro[3.5]nonan-7-yl)-1-methyl-ethyl]-1-piperidyl]-4-fluoro-7-methyl-1-oxo-isoindolin-2-yl]piperidine-2,6-dione (200 mg, 0.38 mmol) and 3-[3-[6-bromo-1-oxo-4-(trifluoromethyl)isoindolin-2-yl]phenyl]-3-[(4-methyl-1,2,4-triazol-3-yl)methyl]cyclobutanecarbonitrile (201 mg, 0.38 mmol) in N,N-dimethylacetamide (10 mL) were added cesium carbonate (371 mg, 1.1 mmol) and XPhos Pd G4 (32 mg, 0.04 mmol). The mixture was stirred at 90° C. for 10 h under nitrogen atmosphere. The reaction mixture was filtered, and concentrated. The residue was purified by prep-TLC (dichloromethane/methanol=10/1), then further purified by prep-HPLC (52%-82% acetonitrile in water (NH₄HCO₃) over 9 min) to afford (1r, 3r)-3-(3-(6-(7-(2-(1-(2-(2,6-dioxopiperidin-3-yl)-4-fluoro-7-methyl-1-oxoisoindolin-5-yl)piperidin-4-yl)propan-2-yl)-2,7-diazaspiro[3.5]nonan-2-yl)-1-oxo-4-(trifluoromethyl)isoindolin-2-yl)phenyl)-3-((4-methyl-4H-1,2,4-triazol-3-yl)methyl)cyclobutane-1-carbonitrile (5.5 mg, 1.5%) as a yellow solid. MS (ESI) m/z: 975.7 [M+H]⁺; ¹H NMR (400 MHz, DMSO-d₆) δ 10.98 (s, 1H), 8.22-8.10 (m, 1H), 7.85 (d, J=9.6 Hz, 1H), 7.43 (s, 1H), 7.34 (t, J=7.6 Hz, 1H), 6.94 (s, 2H), 6.90 (d, J=7.6 Hz, 1H), 6.85-6.78 (m, 1H), 5.03 (dd, J=5.6, 13.2 Hz, 1H), 4.96 (s, 2H), 4.50-4.19 (m, 2H), 3.70 (d, J=0.8 Hz, 4H), 3.64-3.48 (m, 3H), 3.46-3.36 (m, 5H), 3.29-3.15 (m, 4H), 2.94-2.83 (m, 5H), 2.82-2.76 (m, 4H), 2.04-1.90 (m, 2H), 1.87-1.63 (m, 7H), 1.51-1.25 (m, 4H), 1.02-0.74 (m, 6H).

### Example 279: Exemplary synthesis of 3-[4-fluoro-1-oxo-5-(4-{[(1r, 4r)-4-[(3,3-dimethyl-4-{[1-methyl-2-(3-{3-[(4-methyl-4H-1,2,4-triazol-3-yl)methyl]oxetan-3-yl}phenyl)-3-oxo-7-(trifluoromethyl)-2,3-dihydro-1H-isoindol-5-yl]methyl}piperazin-1-yl)methyl]cyclohexyl]oxy}piperidin-1-yl)-2,3-dihydro-1H-isoindol-2-yl]piperidine-2,6-dione

### Step 1: Preparation of potassium ((4-(tert-butoxycarbonyl)-2,2-dimethylpiperazin-1-yl)methyl)trifluoroborate

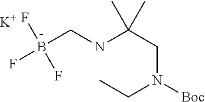

A mixture of tert-butyl 3,3-dimethylpiperazine-1-carboxylate (1.0 g, 4.7 mmol) and potassium vinyltrifluoroborate (893 mg, 4.4 mmol) in tetrahydrofuran (10 mL) was stirred at 80° C. for 3 h, then the mixture was cooled to room temperature and concentrated, followed by the addition of acetone (10 mL) and potassium hydroxide (249 mg, 4.4 mmol). The mixture was stirred at 25° C. for 1 h, then filtered and washed with acetone (10 mL×2). The filtrate solution was concentrated to afford potassium ((4-(tert-butoxycarbonyl)-2,2-dimethylpiperazin-1-yl)methyl)trifluoroborate (952 mg, crude) as an off-white solid.

### Step 2: Preparation of tert-butyl 3,3-dimethyl-4-[[1-methyl-2-[3-[3-[(4-methyl-1,2,4-triazol-3-yl)methyl]oxetan-3-yl]phenyl]-3-oxo-7-(trifluoromethyl)isoindolin-5-yl]methyl]piperazine-1-carboxylate

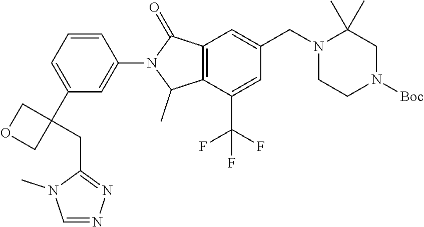 [SMILES: CC1c2c(cc(CN3CCN(C(=O)OC(C)(C)C)CC3(C)C)cc2C(F)(F)F)C(=O)N1c1cccc(C2(Cc3nncn3C)COC2)c1]

To a mixture of 6-bromo-3-methyl-2-[3-[3-[(4-methyl-1,2,4-triazol-3-yl)methyl]oxetan-3-yl]phenyl]-4-(trifluoromethyl)isoindolin-1-one (200 mg, 0.4 mmol) and potassium ((4-(tert-butoxycarbonyl)-2,2-dimethylpiperazin-1-yl)methyl)trifluoroborate (377 mg, 1.1 mmol) in 1,4-dioxane (5.0 mL) and water (1.0 mL) were added palladium acetate (16.9 mg, 75 μmol), bis(1-adamantyl)-butyl-phosphane (54 mg, 0.15 mmol) and potassium carbonate (159 mg, 1.1 mmol). The mixture was stirred at 120° C. in a microwave under nitrogen for 1 h. The mixture was dried over sodium sulfate, filtered, and concentrated. The residue was purified by flash column chromatography (2-7% methanol in dichloromethane) to afford tert-butyl 3,3-dimethyl-4-[[1-methyl-2-[3-[3-[(4-methyl-1,2,4-triazol-3-yl)methyl]oxetan-3-yl]phenyl]-3-oxo-7-(trifluoromethyl)isoindolin-5-yl]methyl]piperazine-1-carboxylate (195 mg, 73% purity, 57%) as a yellow foam. MS (ESI) m/z: 669.4 [M+H]⁺.

### Step 3: Preparation of 6-[(2,2-dimethylpiperazin-1-yl)methyl]-3-methyl-2-[3-[3-[(4-methyl-1,2,4-triazol-3-yl)methyl]oxetan-3-yl]phenyl]-4-(trifluoromethyl)isoindolin-1-one

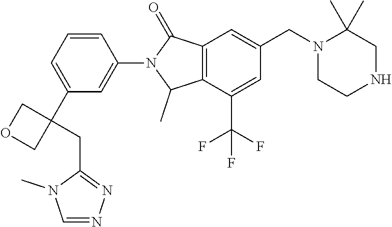 [SMILES: CC1c2c(cc(CN3CCNCC3(C)C)cc2C(F)(F)F)C(=O)N1c1cccc(C2(Cc3nncn3C)COC2)c1]

To a solution of tert-butyl 3,3-dimethyl-4-[[1-methyl-2-[3-[3-[(4-methyl-1,2,4-triazol-3-yl)methyl]oxetan-3-yl]phenyl]-3-oxo-7-(trifluoromethyl)isoindolin-5-yl]methyl]piperazine-1-carboxylate (120 mg, 0.13 mmol) in dichloromethane (2.0 mL) was added TFA (400 μL). The reaction solution was stirred at 25° C. for 1 h, then concentrated. The residue was diluted with acetonitrile and water, then lyophilized to afford 6-[(2,2-dimethylpiperazin-1-yl)methyl]-3-methyl-2-[3-[3-[(4-methyl-1,2,4-triazol-3-yl)methyl]oxetan-3-yl]phenyl]-4-(trifluoromethyl)isoindolin-1-one (153 mg, crude, TFA salt) as a yellow solid. MS (ESI) m/z: 569.5 [M+H]⁺.

### Step 4: Preparation of 3-[5-[4-[4-[[3,3-dimethyl-4-[[1-methyl-2-[3-[3-[(4-methyl-1,2,4-triazol-3-yl)methyl]oxetan-3-yl]phenyl]-3-oxo-7-(trifluoromethyl)isoindolin-5-yl]methyl]piperazin-1-yl]methyl]cyclohexoxy]-1-piperidyl]-4-fluoro-1-oxo-isoindolin-2-yl]piperidine-2,6-dione

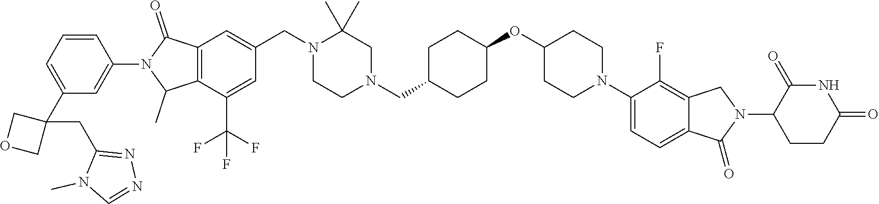 [SMILES: CC1c2c(cc(CN3CCN(C[C@H]4CC[C@H](OC5CCN(c6ccc7c(c6F)CN(C6CCC(=O)NC6=O)C7=O)CC5)CC4)CC3(C)C)cc2C(F)(F)F)C(=O)N1c1cccc(C2(Cc3nncn3C)COC2)c1]

To a mixture of 6-[(2,2-dimethylpiperazin-1-yl)methyl]-3-methyl-2-[3-[3-[(4-methyl-1,2,4-triazol-3-yl)methyl]oxetan-3-yl]phenyl]-4-(trifluoromethyl)isoindolin-1-one trifluoroacetate (153 mg, 141 μmol) and sodium acetate (58 mg, 0.7 mmol) in dichloromethane (2.0 mL) and DMSO (2.0 mL) was dropwise added 4-[[1-[2-(2,6-dioxo-3-piperidyl)-4-fluoro-1-oxo-isoindolin-5-yl]-4-piperidyl]oxy]cyclohexanecarbaldehyde (80 mg, 0.16 mmol) in dichloromethane (2.0 mL) at 0° C. The mixture was stirred at 0° C. for 1 h, followed by the addition of NaBH(OAc)₃ (75 mg, 0.35 mmol). The reaction mixture was stirred at 0° C. for 1 h, then concentrated. The residue was diluted with DMF (3 mL) and filtered. The filtrate was purified by prep-HPLC (42%-82% acetonitrile in water (NH₄HCO₃) over 32 min at 60 mL/min) to afford 3-[5-[4-[4-[[3,3-dimethyl-4-[[1-methyl-2-[3-[3-[(4-methyl-1,2,4-triazol-3-yl)methyl]oxetan-3-yl]phenyl]-3-oxo-7-(trifluoromethyl)isoindolin-5-yl]methyl]piperazin-1-yl]methyl]cyclohexoxy]-1-piperidyl]-4-fluoro-1-oxo-isoindolin-2-yl]piperidine-2,6-dione (33.9 mg, 23%) as a white solid. MS (ESI) m/z: 1024.4 [M+H]⁺; ¹H NMR (400 MHz, DMSO-d₆) δ 11.08-10.88 (m, 1H), 8.19 (br s, 1H), 8.07-7.87 (m, 2H), 7.65-7.53 (m, 1H), 7.50-7.33 (m, 2H), 7.27-7.10 (m, 2H), 6.85 (br d, J=4.4 Hz, 1H), 5.72-5.54 (m, 1H), 5.11-5.01 (m, 1H), 5.00-4.81 (m, 4H), 4.54-4.41 (m, 1H), 4.30 (br d, J=16.8 Hz, 1H), 3.65-3.49 (m, 6H), 2.90 (br s, 7H), 2.60 (br s, 2H), 2.36 (br s, 4H), 2.27-2.07 (m, 3H), 2.00 (br s, 3H), 1.92 (br d, J=2.0 Hz, 4H), 1.78 (br dd, J=2.0, 6.8 Hz, 2H), 1.56 (br s, 2H), 1.45-1.37 (m, 1H), 1.26 (br s, 4H), 1.11 (br s, 7H), 0.93-0.80 (m, 2H).

### Example 280: Exemplary synthesis of 3-[7-methyl-5-oxo-2-(4-{[(1r, 4r)-4-[(4-{[2-(3-{3,3-dimethyl-1-[(4-methyl-4H-1,2,4-triazol-3-yl)methyl]cyclobutyl}phenyl)-3-oxo-7-(trifluoromethyl)-2,3-dihydro-1H-isoindol-5-yl]methyl}-3,3-dimethylpiperazin-1-yl)methyl]cyclohexyl]oxy}piperidin-1-yl)-5H, 6H, 7H-pyrrolo[3,4-b]pyridin-6-yl]piperidine-2,6-dione

### Step 1: Preparation of ethyl 2-(3,3-dimethylcyclobutylidene)acetate

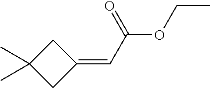

To a solution of ethyl 2-(triphenyl-phosphanylidene)acetate (2.90 g, 8.3 mmol) in dichloromethane (3 mL) and N,N-dimethylformamide (6 mL) was added 3,3-dimethylcyclobutanone (800 mg, 8.2 mmol) at 0° C. I mixture was stirred for 12 h at 60° C. The reaction mixture was diluted with water (30 mL) and extracted with ethyl acetate (30 mL×3). The combined organic layers were washed with brine (30 mL×3), dried over anhydrous sodium sulfate, filtered, and concentrated under reduced pressure. The residue was purified by silica gel column chromatography (petroleum ether/ethyl acetate=1:0 to 5:1) to afford ethyl 2-(3,3-dimethylcyclobutylidene)acetate (450 mg, 33%) as a yellow oil. ¹H NMR (400 MHz, CDCl₃) δ 5.51-5.68 (m, 1H), 4.03-4.11 (m, 2H), 2.77 (d, J=2.0 Hz, 2H), 2.47 (s, 2H), 1.18-1.22 (m, 3H), 1.13 (s, 6H).

### Step 2: Preparation of ethyl 2-[1-[3-(tert-butoxycarbonylamino)phenyl]-3,3-dimethyl-cyclobutyl]acetate

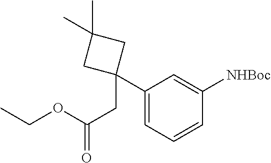 [SMILES: CCOC(=O)CC1(c2cccc(NC(=O)OC(C)(C)C)c2)CC(C)(C)C1]

To a solution of ethyl 2-(3,3-dimethylcyclobutylidene)acetate (450 mg, 2.7 mmol) in 1,4-dioxane (10 mL) were added [3-(tert-butoxycarbonylamino)phenyl]boronic acid (824 mg, 3.5 mmol) and potassium hydroxide (1.5 M, 2 mL) at 0° C. The mixture was stirred at 0° C. for 0.5 h before the addition of chloro(1,5-cyclooctadiene)rhodium(I) dimer (13 mg, 0.03 mmol) at 0° C. The reaction mixture was stirred at 25° C. for 10 h under nitrogen, then concentrated under reduced pressure. The residue was purified by silica gel column chromatography (petroleum ether/ethyl acetate=30:1 to 3:1) to afford ethyl 2-[1-[3-(tert-butoxycarbonylamino)phenyl]-3,3-dimethyl-cyclobutyl]acetate (800 mg, 83%) as a yellow oil. MS (ESI) m/z: 363.3 [M+H]⁺; ¹H NMR (400 MHz, CDCl₃) δ 7.26-7.30 (m, 1H), 7.22 (d, J=7.2 Hz, 1H), 7.15 (s, 1H), 6.85 (d, J=6.8 Hz, 1H), 6.42 (s, 1H), 3.85 (q, J=7.2 Hz, 2H), 2.62 (s, 2H), 2.18-2.36 (m, 4H), 1.44 (s, 9H), 1.11 (s, 3H), 1.00 (t, J=7.2 Hz, 3H), 0.86 (s, 3H).

### Step 3: Preparation of 2-[1-[3-(tert-butoxycarbonylamino)phenyl]-3,3-dimethyl-cyclobutyl]acetic acid

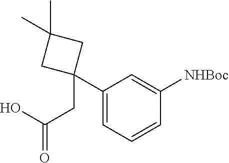 [SMILES: CC1(C)CC(CC(=O)O)(c2cccc(NC(=O)OC(C)(C)C)c2)C1]

To a solution of ethyl 2-[1-[3-(tert-butoxycarbonylamino)phenyl]-3,3-dimethyl-cyclobutyl]acetate (950 mg, 2.6 mmol) in methanol (9 mL) was added lithium hydroxide (331 mg, 7.9 mmol) in water (3 mL). The mixture was stirred at 25° C. for 2 h, then concentrated under reduced pressure. 1 M aqueous hydrochloric acid solution was added to the residue to adjust the pH to 5-6, and the resulting solution was concentrated to afford 2-[1-[3-(tert-butoxycarbonylamino)phenyl]-3,3-dimethyl-cyclobutyl]acetic acid (860 mg, crude) as a white solid. ¹H NMR (400 MHz, CDCl₃) δ 7.36-7.40 (m, 1H), 7.32 (d, J=7.2 Hz, 1H), 7.21-7.25 (m, 2H), 6.88-6.99 (m, 1H), 2.78 (s, 2H), 2.39-2.48 (m, 2H), 2.30-2.36 (m, 2H), 1.54 (s, 9H), 1.22 (s, 3H), 0.97 (s, 3H).

### Step 4: Preparation of tert-butyl N-[3-[3,3-dimethyl-1-[2-[2-(methylcarbamothioyl)hydrazino]-2-oxo-ethyl]cyclobutyl]phenyl]carbamate

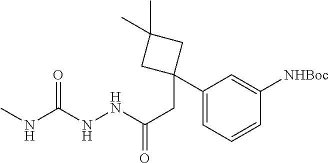 [SMILES: CNC(=O)NNC(=O)CC1(c2cccc(NC(=O)OC(C)(C)C)c2)CC(C)(C)C1]

To a solution of 2-[1-[3-(tert-butoxycarbonylamino)phenyl]-3,3-dimethyl-cyclobutyl]acetic acid (860 mg, 2.6 mmol) in N,N-dimethylformamide (5 mL) were added N,N-diisopropylethylamine (667 mg, 5.2 mmol), 0-(7-Azabenzotriazol-1-yl)-N,N,N′,N′-tetramethyluronium Hexafluorophosphate (1.27 g, 3.4 mmol) and 1-amino-3-methyl-thiourea (407 mg, 3.9 mmol). The mixture was stirred at 25° C. for 5 h, then diluted with water (30 mL) and extracted with ethyl acetate (30 mL×3). The combined organic layers were washed with brine (30 mL×3), dried over anhydrous sodium sulfate, filtered, and concentrated under reduced pressure to afford tert-butyl N-[3-[3,3-dimethyl-1-[2-[2-(methylcarbamothioyl)hydrazino]-2-oxo-ethyl]cyclobutyl]phenyl]carbamate (1.00 g, 92%) as a yellow oil, which was used in the next step without further purification. MS (ESI) m/z: 421.2 [M+H]⁺.

### Step 5: Preparation of tert-butyl N-[3-[3,3-dimethyl-1-[(4-methyl-5-sulfanyl-1,2,4-triazol-3-yl)methyl]cyclobutyl]phenyl]carbamate

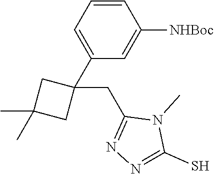 [SMILES: Cn1c(S)nnc1CC1(c2cccc(NC(=O)OC(C)(C)C)c2)CC(C)(C)C1]

To a solution of tert-butyl N-[3-[3,3-dimethyl-1-[2-[2-(methylcarbamothioyl)hydrazino]-2-oxo-ethyl]cyclobutyl]phenyl]carbamate (1.00 g, 2.4 mmol) in tetrahydrofuran (15 mL) was added sodium hydroxide (1 M, 4 mL), and the mixture was stirred at 50° C. for 10 h. The reaction mixture was concentrated under reduced pressure to afford tert-butyl N-[3-[3,3-dimethyl-1-[(4-methyl-5-sulfanyl-1,2,4-triazol-3-yl)methyl]cyclobutyl]phenyl]carbamate (950 mg, crude) as a yellow oil. MS (ESI) m/z: 403.2 [M+H]⁺.

### Step 6: Preparation of tert-butyl N-[3-[3,3-dimethyl-1-[(4-methyl-1,2,4-triazol-3-yl)methyl]cyclobutyl]phenyl]carbamate

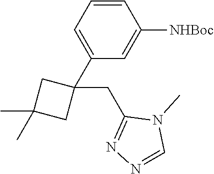

To a solution of tert-butyl N-[3-[3,3-dimethyl-1-[(4-methyl-5-sulfanyl-1,2,4-triazol-3-yl)methyl]cyclobutyl]phenyl]carbamate (450 mg, 1.1 mmol) in dichloromethane (5 mL) were added acetic acid (940 mg, 16 mmol) and 30% hydrogen peroxide (127 mg, 1.1 mmol) at 0° C. The mixture was stirred at 25° C. for 1 h, then diluted with water (40 mL) and extracted with dichloromethane (20 mL×3). The combined organic layers were washed with brine (35 mL), dried over anhydrous sodium sulfate, filtered, and concentrated under reduced pressure. The residue was purified by prep-HPLC (35%-65% acetonitrile in water (formic acid) over 15 min) to afford tert-butyl N-[3-[3,3-dimethyl-1-[(4-methyl-1,2,4-triazol-3-yl)methyl]cyclobutyl]phenyl]carbamate (169 mg, 41%) as a white solid. MS (ESI) m/z: 371.2 [M+H]⁺.

### Step 7: Preparation of 3-[3,3-dimethyl-1-[(4-methyl-1,2,4-triazol-3-yl)methyl]cyclobutyl]aniline

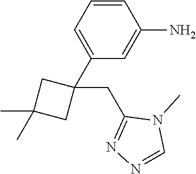

To a solution of tert-butyl N-[3-[3,3-dimethyl-1-[(4-methyl-1,2,4-triazol-3-yl)methyl]cyclobutyl]phenyl]carbamate (169 mg, 0.5 mmol) in dichloromethane (0.5 mL) was added trifluoroacetic acid (156 mg, 1.4 mmol). The mixture was stirred at 25° C. for 0.5 h, then concentrated under reduced pressure. The residue was purified by prep-HPLC (0-30% acetonitrile in water (TFA) over 9 min) to afford 3-[3,3-dimethyl-1-[(4-methyl-1,2,4-triazol-3-yl)methyl]cyclobutyl]aniline (170 mg, 97%, TFA salt) as a white solid. MS (ESI) m/z: 271.1 [M+H]⁺.

### Step 8: Preparation of 6-bromo-2-[3-[3,3-dimethyl-1-[(4-methyl-1,2,4-triazol-3-yl)methyl]cyclobutyl]phenyl]-4-(trifluoromethyl)isoindolin-1-one

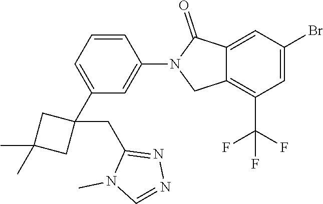 [SMILES: Cn1cnnc1CC1(c2cccc(N3Cc4c(cc(Br)cc4C(F)(F)F)C3=O)c2)CC(C)(C)C1]

A solution of 3-[3,3-dimethyl-1-[(4-methyl-1,2,4-triazol-3-yl)methyl]cyclobutyl]aniline trifluoroacetate (330 mg, 0.86 mmol) and methyl 5-bromo-2-(bromomethyl)-3-(trifluoromethyl)benzoate (323 mg, 0.86 mmol) in acetonitrile (8 mL) was cooled to 0° C. before the addition of silver nitrate (219 mg, 1.3 mmol) in water (2 mL). The mixture was stirred at 25° C. for 12 h, then diluted with sodium carbonate until pH 9. The resulting mixture was filtered and concentrated under reduced pressure. The residue was diluted with water (50 mL) and extracted with ethyl acetate (30 mL×3). The combined organic layers were washed with brine (50 mL), dried over anhydrous sodium sulfate, filtered, and concentrated under reduced pressure. The residue was purified by silica gel column chromatography (petroleum ether/ethyl acetate=1:1 then dichloromethane/methanol=20:1) to afford 6-bromo-2-[3-[3,3-dimethyl-1-[(4-methyl-1,2,4-triazol-3-yl)methyl]cyclobutyl]phenyl]-4-(trifluoromethyl)isoindolin-1-one (170 mg, 37%) as a yellow solid. MS (ESI) m/z: 535.0 [M+H]⁺.

### Step 9: Preparation of 2-[3-[3,3-dimethyl-1-[(4-methyl-1,2,4-triazol-3-yl)methyl]cyclobutyl]phenyl]-6-(hydroxymethyl)-4-(trifluoromethyl)isoindolin-1-one

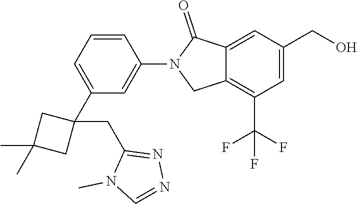 [SMILES: Cn1cnnc1CC1(c2cccc(N3Cc4c(cc(CO)cc4C(F)(F)F)C3=O)c2)CC(C)(C)C1]

A mixture of 6-bromo-2-[3-[3,3-dimethyl-1-[(4-methyl-1,2,4-triazol-3-yl)methyl]cyclobutyl]phenyl]-4-(trifluoromethyl)isoindolin-1-one (170 mg, 0.3 mmol), tributylstannylmethanol (153 mg, 0.5 mmol) and Xphos Pd G4 (27 mg, 0.03 mmol) in 1,4-dioxane (3 mL) was degassed and purged with nitrogen several times, and the mixture was stirred at 80° C. for 10 h under nitrogen atmosphere. The reaction mixture was diluted with potassium fluoride solution (50 mL) and extracted with ethyl acetate (30 mL×3). The combined organic layers were washed with brine (30 mL×3), dried over anhydrous sodium sulfate, filtered, and concentrated under reduced pressure. The residue was purified by silica gel column chromatography (petroleum ether/ethyl acetate=1:1 then dichloromethane/methanol=20:1) to afford 2-[3-[3,3-dimethyl-1-[(4-methyl-1,2,4-triazol-3-yl)methyl]cyclobutyl]phenyl]-6-(hydroxymethyl)-4-(trifluoromethyl)isoindolin-1-one (80 mg, 52%) as a yellow solid. MS (ESI) m/z: 485.2 [M+H]⁺.

### Step 10: Preparation of 6-(chloromethyl)-2-[3-[3,3-dimethyl-1-[(4-methyl-1,2,4-triazol-3-yl)methyl]cyclobutyl]phenyl]-4-(trifluoromethyl)isoindolin-1-one

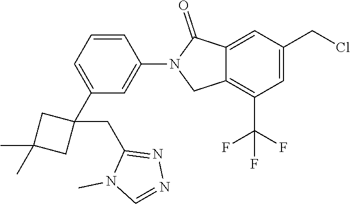 [SMILES: Cn1cnnc1CC1(c2cccc(N3Cc4c(cc(CCl)cc4C(F)(F)F)C3=O)c2)CC(C)(C)C1]

To a solution of 2-[3-[3,3-dimethyl-1-[(4-methyl-1,2,4-triazol-3-yl)methyl]cyclobutyl]phenyl]-6-(hydroxymethyl)-4-(trifluoromethyl)isoindolin-1-one (60 mg, 0.1 mmol) in dichloromethane (2 mL) was added thionyl chloride (164 mg, 1.4 mmol). The mixture was stirred at 25° C. for 0.5 h, then concentrated to afford 6-(chloromethyl)-2-[3-[3,3-dimethyl-1-[(4-methyl-1,2,4-triazol-3-yl)methyl]cyclobutyl]phenyl]-4-(trifluoromethyl)isoindolin-1-one (60 mg, crude) as a yellow oil. MS (ESI) m/z: 503.2 [M+H]⁺.

### Step 11: Preparation of tert-butyl 4-[[2-[3-[3,3-dimethyl-1-[(4-methyl-1,2,4-triazol-3-yl)methyl]cyclobutyl]phenyl]-3-oxo-7-(trifluoromethyl)isoindolin-5-yl]methyl]-3,3-dimethyl-piperazine-1-carboxylate

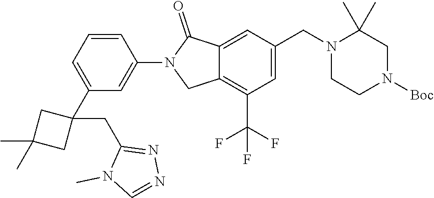 [SMILES: Cn1cnnc1CC1(c2cccc(N3Cc4c(cc(CN5CCN(C(=O)OC(C)(C)C)CC5(C)C)cc4C(F)(F)F)C3=O)c2)CC(C)(C)C1]

To a solution of 6-(chloromethyl)-2-[3-[3,3-dimethyl-1-[(4-methyl-1,2,4-triazol-3-yl)methyl]cyclobutyl]phenyl]-4-(trifluoromethyl)isoindolin-1-one (60 mg, 0.1 mmol) in acetonitrile (2 mL) were added N,N-diisopropylethylamine (77 mg, 0.6 mmol) and tert-butyl 3,3-dimethylpiperazine-1-carboxylate (51 mg, 0.2 mmol). The mixture was stirred at 60° C. for 0.5 h, then concentrated under reduced pressure. The residue was purified by prep-HPLC (23%-53% acetonitrile in water (formic acid) over 10 min) to afford tert-butyl 4-[[2-[3-[3,3-dimethyl-1-[(4-methyl-1,2,4-triazol-3-yl)methyl]cyclobutyl]phenyl]-3-oxo-7-(trifluoromethyl)isoindolin-5-yl]methyl]-3,3-dimethyl-piperazine-1-carboxylate (40 mg, 49%) as a white solid. MS (ESI) m/z: 681.5 [M+H]⁺.

### Step 12: Preparation of 2-[3-[3,3-dimethyl-1-[(4-methyl-1,2,4-triazol-3-yl)methyl]cyclobutyl]phenyl]-6-[(2,2-dimethylpiperazin-1-yl)methyl]-4-(trifluoromethyl)isoindolin-1-one

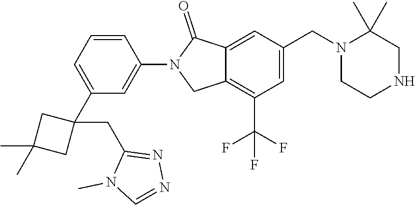 [SMILES: Cn1cnnc1CC1(c2cccc(N3Cc4c(cc(CN5CCNCC5(C)C)cc4C(F)(F)F)C3=O)c2)CC(C)(C)C1]

To a solution of tert-butyl 4-[[2-[3-[3,3-dimethyl-1-[(4-methyl-1,2,4-triazol-3-yl)methyl]cyclobutyl]phenyl]-3-oxo-7-(trifluoromethyl)isoindolin-5-yl]methyl]-3,3-dimethyl-piperazine-1-carboxylate (40 mg, 0.06 mmol) in dichloromethane (2 mL) was added trifluoroacetic acid (768 mg, 6.7 mmol). The reaction solution was stirred at 25° C. for 0.5 h, then concentrated under reduced pressure to afford 2-[3-[3,3-dimethyl-1-[(4-methyl-1,2,4-triazol-3-yl)methyl]cyclobutyl]phenyl]-6-[(2,2-dimethylpiperazin-1-yl)methyl]-4-(trifluoromethyl)isoindolin-1-one (40 mg, crude, TFA salt) as a yellow oil. MS (ESI) m/z: 581.4 [M+H]⁺.

### Step 13: Preparation of 3-[2-[4-[4-[[4-[[2-[3-[3,3-dimethyl-1-[(4-methyl-1,2,4-triazol-3-yl)methyl]cyclobutyl]phenyl]-3-oxo-7-(trifluoromethyl)isoindolin-5-yl]methyl]-3,3-dimethyl-piperazin-1-yl]methyl]cyclohexoxy]-1-piperidyl]-7-methyl-5-oxo-7H-pyrrolo[3,4-b]pyridin-6-yl]piperidine-2,6-dione

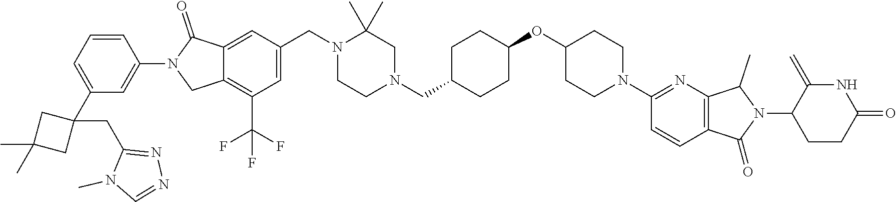 [SMILES: C=C1NC(=O)CCC1N1C(=O)c2ccc(N3CCC(O[C@H]4CC[C@H](CN5CCN(Cc6cc7c(c(C(F)(F)F)c6)CN(c6cccc(C8(Cc9nncn9C)CC(C)(C)C8)c6)C7=O)C(C)(C)C5)CC4)CC3)nc2C1C]

To a solution of 2-[3-[3,3-dimethyl-1-[(4-methyl-1,2,4-triazol-3-yl)methyl]cyclobutyl]phenyl]-6-[(2,2-dimethylpiperazin-1-yl)methyl]-4-(trifluoromethyl)isoindolin-1-one trifluoroacetate (40 mg, 0.06 mmol) in dichloromethane (3 mL) were added N-methylmorpholine (29 mg, 0.3 mmol), 4-[[1-[6-(2,6-dioxo-3-piperidyl)-7-methyl-5-oxo-7H-pyrrolo[3,4-b]pyridin-2-yl]-4-piperidyl]oxy]cyclohexanecarbaldehyde (27 mg, 0.06 mmol) and sodium triacetoxyborohydride (37 mg, 0.2 mmol). The mixture was stirred at 0° C. for 0.5 h, then concentrated under reduced pressure. The residue was purified by prep-HPLC (20%-50% acetonitrile in water (formic acid) over 10 min) to afford 3-[2-[4-[4-[[4-[[2-[3-[3,3-dimethyl-1-[(4-methyl-1,2,4-triazol-3-yl)methyl]cyclobutyl]phenyl]-3-oxo-7-(trifluoromethyl)isoindolin-5-yl]methyl]-3,3-dimethyl-piperazin-1-yl]methyl]cyclohexoxy]-1-piperidyl]-7-methyl-5-oxo-7H-pyrrolo[3,4-b]pyridin-6-yl]piperidine-2,6-dione (33.5 mg, 55%) as a white solid. MS (ESI) m/z: 1033.5 [M+H]⁺; ¹H NMR (400 MHz, DMSO-d₆) δ 10.89 (d, J=12.4 Hz, 1H), 8.13 (s, 1H), 7.98-8.11 (m, 1H), 7.94 (s, 1H), 7.76 (d, J=7.6 Hz, 1H), 7.70 (m, 1H), 7.46 (s, 1H), 7.33 (t, J=7.6 Hz, 1H), 6.89 (m, 1H), 6.83 (m, 1H), 5.09 (s, 2H), 4.70 (m, 1H), 4.39-4.51 (m, 1H), 3.97-4.31 (m, 3H), 3.69-3.75 (m, 1H), 3.38-3.42 (m, 2H), 3.24-3.29 (m, 2H), 3.12 (s, 2H), 2.96-3.07 (m, 1H), 2.83-2.95 (m, 2H), 2.71-2.83 (m, 2H), 2.68-2.71 (m, 3H), 2.60 (m, 1H), 2.54 (d, J=2.0 Hz, 2H), 2.45-2.47 (m, 1H), 2.25-2.34 (m, 3H), 2.05-2.15 (m, 1H), 1.92-2.04 (m, 4H), 1.86 (m, 2H), 1.62-1.82 (m, 3H), 1.49-1.59 (m, 1H), 1.35-1.45 (m, 6H), 1.26 (d, J=8.8 Hz, 3H), 1.22 (s, 3H), 1.20 (s, 1H), 1.10-1.18 (m, 3H), 1.03-1.09 (m, 1H), 0.93 (s, 3H), 0.79-0.90 (m, 1H).

### Example 281: Exemplary synthesis of 3-[7-methyl-5-oxo-2-(4-{[(1r, 4r)-4-({3,3-dimethyl-4-[(2-{3-[2-(4-methyl-4H-1,2,4-triazol-3-yl)oxetan-2-yl]phenyl}-3-oxo-7-(trifluoromethyl)-2,3-dihydro-1H-isoindol-5-yl)methyl]piperazin-1-yl}methyl)cyclohexyl]oxy}piperidin-1-yl)-5H, 6H, 7H-pyrrolo[3,4-b]pyridin-6-yl]piperidine-2,6-dione

### Step 1: Preparation of 6-(hydroxymethyl)-4-(trifluoromethyl)isoindolin-1-one

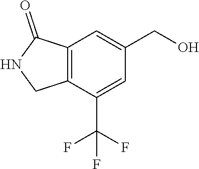 [SMILES: O=C1NCc2c1cc(CO)cc2C(F)(F)F]

To a stirred solution of 7 M NH₃/methanol (10 mL) in methanol (20 mL) was added methyl 2-(bromomethyl)-5-(hydroxymethyl)-3-(trifluoromethyl)benzoate (2.0 g, 6 mmol) in a Teflon reactor. The reactor was sealed and stirred at 50° C. for 15 h. The reaction mixture was concentrated under reduced pressure. The residue was purified by flash column chromatography (0-5% methanol in dichloromethane) to afford 6-(hydroxymethyl)-4-(trifluoromethyl)isoindolin-1-one (1.1 g, 76%) as a white solid. MS (ESI) m/z: 231.8 [M+H]⁺.

### Step 2: Preparation of 6-(chloromethyl)-4-(trifluoromethyl)isoindolin-1-one

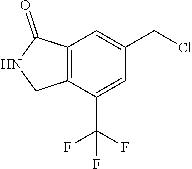 [SMILES: O=C1NCc2c1cc(CCl)cc2C(F)(F)F]

To a stirred solution of 6-(hydroxymethyl)-4-(trifluoromethyl)isoindolin-1-one (1.1 g, 4.7 mmol) in dichloromethane (10 mL) was added thionyl chloride (4.0 mL, 55 mmol). The reaction solution was stirred at 25° C. for 2 h, then concentrated under reduced pressure to afford 6-(chloromethyl)-4-(trifluoromethyl)isoindolin-1-one (1.2 g, crude, HCl salt) as a white solid. MS (ESI) m/z: 250.1 [M+H]⁺.

### Step 3: Preparation of tert-butyl 3,3-dimethyl-4-[[3-oxo-7-(trifluoromethyl)isoindolin-5-yl]methyl]piperazine-1-carboxylate

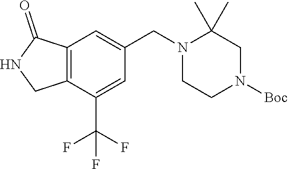 [SMILES: CC(C)(C)OC(=O)N1CCN(Cc2cc3c(c(C(F)(F)F)c2)CNC3=O)C(C)(C)C1]

A mixture of 6-(chloromethyl)-4-(trifluoromethyl)isoindolin-1-one (1.24 g, 5 mmol), tert-butyl 3,3-dimethylpiperazine-1-carboxylate (1.2 g, 5 mmol) and N,N-diisopropylethylamine (3.5 mL, 20 mmol) in DMSO (5.0 mL) was stirred at 80° C. for 16 h. The reaction mixture was diluted with water (30 mL) and extracted with dichloromethane (30 mL×3). The combined organic layer was washed with brine (30 mL), dried over sodium sulfate, filtered, and concentrated. The residue was purified by flash column chromatography (0-4% methanol in dichloromethane) to afford tert-butyl 3,3-dimethyl-4-[[3-oxo-7-(trifluoromethyl)isoindolin-5-yl]methyl]piperazine-1-carboxylate (1.8 g, 75%) as a yellow solid. MS (ESI) m/z: 428.0 [M+H]⁺.

### Step 4: Preparation of methyl 2-(3-bromophenyl)-2-diazo-acetate

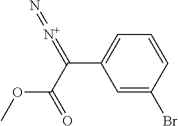

To a solution of methyl 2-(3-bromophenyl)acetate (3.0 g, 13 mmol) and N-diazo-4-methyl-benzenesulfonamide (4.13 g, 16 mmol) in acetonitrile (30 mL) was dropwise added DBU (2.96 mL, 20 mmol) at 0° C., and the resulting mixture was stirred at 25° C. for 16 h. The mixture was diluted with saturated ammonium chloride solution (20 mL) and extracted with ethyl acetate (30 mL×3). The combined organic layer was washed with brine (20 mL), dried over sodium sulfate, filtered, and concentrated. The residue was triturated with petroleum ether/ethyl acetate (20/1, 100 mL) to afford methyl 2-(3-bromophenyl)-2-diazo-acetate (3.85 g, crude) as a yellow solid. MS (ESI) m/z: 267.9 [M-N₂+ACN]⁺; ¹H NMR (400 MHz, CDCl₃) δ 7.69 (t, J=2.0 Hz, 1H), 7.39 (td, J=1.2, 7.6 Hz, 1H), 7.32-7.29 (m, 1H), 7.26-7.22 (m, 1H), 3.88 (s, 3H).

### Step 5: Preparation of methyl 2-(2-bromoethoxy)-2-(3-bromophenyl)acetate

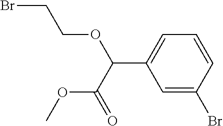 [SMILES: COC(=O)C(OCCBr)c1cccc(Br)c1]

To a mixture of 2-bromoethanol (1.49 g, 12 mmol) and Rh₂(OAc)₄ (528 mg, 1.2 mmol) in dichloromethane (20 mL) was dropwise added methyl 2-(3-bromophenyl)-2-diazo-acetate (3.85 g, 12 mmol) in dichloromethane (20 mL) at 0° C. The mixture was stirred at 25° C. for 1 h, then filtered and concentrated. The residue was purified by flash column chromatography (0-8% ethyl acetate in petroleum ether) to afford methyl 2-(2-bromoethoxy)-2-(3-bromophenyl)acetate (1.75 g, 36%) as a yellow oil. MS (ESI) m/z: 374.7 [M+Na]⁺; ¹H NMR (400 MHz, CDCl₃) δ 7.63 (s, 1H), 7.49 (d, J=8.0 Hz, 1H), 7.40 (d, J=7.6 Hz, 1H), 7.27-7.23 (m, 1H), 4.94 (s, 1H), 3.91 (td, J=6.4, 10.4 Hz, 1H), 3.79-3.74 (m, 4H), 3.56-3.49 (m, 2H).

### Step 6: Preparation of methyl 2-(3-bromophenyl)oxetane-2-carboxylate

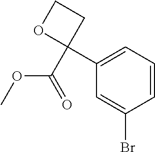

To a mixture of sodium hydride (209 mg, 5 mmol, 60%) in DMF (2 mL) was dropwise added methyl 2-(2-bromoethoxy)-2-(3-bromophenyl)acetate (1.75 g, 5 mmol) in DMF (15 mL) at 0° C. under nitrogen atmosphere. The mixture was stirred at 25° C. for 2 h, then poured into ice water (40 mL) and extracted with ethyl acetate (50 mL×3). The combined organic layer was washed with brine (50 mL), dried over sodium sulfate, filtered, and concentrated to afford methyl 2-(3-bromophenyl)oxetane-2-carboxylate (1.66 g, crude) as a yellow oil. MS (ESI) m/z: 293.0 [M+Na]⁺; ¹H NMR (400 MHz, CDCl₃) δ 7.67 (t, J=1.8 Hz, 1H), 7.46 (dd, J=1.6, 8.0 Hz, 1H), 7.43-7.39 (m, 1H), 7.27-7.23 (m, 1H), 4.68 (td, J=6.4, 8.8 Hz, 1H), 4.58 (td, J=6.4, 8.8 Hz, 1H), 3.78 (s, 3H), 3.44-3.37 (m, 1H), 2.88-2.83 (m, 1H).

### Step 7: Preparation of 2-(3-bromophenyl)oxetane-2-carboxylic acid

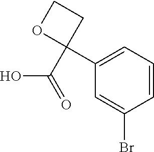 [SMILES: O=C(O)C1(c2cccc(Br)c2)CCO1]

To a solution of methyl 2-(3-bromophenyl)oxetane-2-carboxylate (1.66 g, 4 mmol) in acetonitrile (15 mL) was added 3 M sodium hydroxide (15 mL). The mixture was stirred at 65° C. for 1 h. The pH of the mixture was adjusted to 4-5 by the addition of citric acid, and the resulting mixture was diluted with water (20 mL) and extracted with ethyl acetate (40 mL×3). The combined organic layer was washed with brine (40 mL), dried over sodium sulfate, filtered, and concentrated to afford 2-(3-bromophenyl)oxetane-2-carboxylic acid (1.11 g, crude) as a light yellow oil. ¹H NMR (400 MHz, CDCl₃) δ 7.72 (d, J=1.6 Hz, 1H), 7.52-7.46 (m, 2H), 7.32-7.27 (m, 1H), 4.80-4.71 (m, 1H), 4.69-4.61 (m, 1H), 3.37 (ddd, J=6.8, 8.8, 11.6 Hz, 1H), 3.03-2.92 (m, 1H).

### Step 8: Preparation of 1-[[2-(3-bromophenyl)oxetane-2-carbonyl]amino]-3-methyl-thiourea

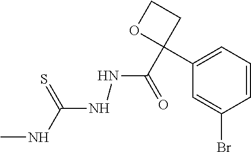 [SMILES: CNC(=S)NNC(=O)C1(c2cccc(Br)c2)CCO1]

To a mixture of 2-(3-bromophenyl)oxetane-2-carboxylic acid (1.11 g, 4 mmol), 1-amino-3-methyl-thiourea (620 mg, 6 mmol) and N,N-diisopropylethylamine (1.52 g, 12 mmol) in dichloromethane (15 mL) was added T₄P (3.68 g, 5 mmol, 50%) at 0° C. The mixture was stirred at 25° C. for 12 h. The pH of the reaction mixture was adjusted to 8-9 by the addition of saturated sodium bicarbonate solution. The resulting mixture was diluted with water (20 mL) and extracted with ethyl acetate (40 mL×3). The combined organic layer was washed with brine (50 mL), dried over sodium sulfate, filtered, and concentrated to afford 1-[[2-(3-bromophenyl)oxetane-2-carbonyl]amino]-3-methyl-thiourea (1.17 g, crude) as gray solid. MS (ESI) m/z: 344.0 [M+H]⁺.

### Step 9: Preparation of 5-[2-(3-bromophenyl)oxetan-2-yl]-4-methyl-1,2,4-triazole-3-thiol

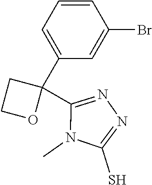 [SMILES: Cn1c(S)nnc1C1(c2cccc(Br)c2)CCO1]

A solution of 1-[[2-(3-bromophenyl)oxetane-2-carbonyl]amino]-3-methyl-thiourea (1.17 g, 3 mmol) in 1 M sodium hydroxide (15 mL) was stirred at 50° C. for 1 h. The pH of the mixture was adjusted to 4-5 by 1 M citric acid, and the resulting mixture was diluted with water (20 mL) and extracted with dichloromethane (30 mL×3). The combined organic layer was washed with brine (40 mL), dried over sodium sulfate, filtered, and concentrated to afford 5-[2-(3-bromophenyl)oxetan-2-yl]-4-methyl-1,2,4-triazole-3-thiol (848 mg, crude) as a yellow gum. MS (ESI) m/z: 328.0 [M+H]⁺.

### Step 10: Preparation of 3-[2-(3-bromophenyl)oxetan-2-yl]-4-methyl-1,2,4-triazole

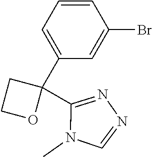 [SMILES: Cn1cnnc1C1(c2cccc(Br)c2)CCO1]

To a solution of 5-[2-(3-bromophenyl)oxetan-2-yl]-4-methyl-1,2,4-triazole-3-thiol (848 mg, 2.4 mmol) and acetic acid (1.47 g, 24 mmol) in dichloromethane (10 mL) was dropwise added 30% hydrogen peroxide (830 mg, 7.3 mmol) at 0° C. The mixture was stirred at 25° C. for 1 h, then poured into saturated sodium bicarbonate solution (20 mL) and sodium sulfite solution (15% wt, 20 mL). The resulting mixture was extracted with dichloromethane (40 mL×3). The combined organic layer was washed with brine (50 mL), dried over sodium sulfate, filtered, and concentrated. The residue was purified by flash column chromatography (0-3% methanol in dichloromethane) to afford 3-[2-(3-bromophenyl)oxetan-2-yl]-4-methyl-1,2,4-triazole (460 mg, 63%) as a yellow gum. MS (ESI) m/z: 294.0 [M+H]⁺; ¹H NMR (400 MHz, CDCl₃) δ 8.08 (s, 1H), 7.69 (s, 1H), 7.50-7.45 (m, 1H), 7.29-7.26 (m, 2H), 4.83-4.71 (m, 2H), 4.14-4.06 (m, 1H), 3.33 (s, 3H), 3.01-2.89 (m, 1H).

### Step 11: Preparation of tert-butyl 3,3-dimethyl-4-[[2-[3-[2-(4-methyl-1,2,4-triazol-3-yl)oxetan-2-yl]phenyl]-3-oxo-7-(trifluoromethyl)isoindolin-5-yl]methyl]piperazine-1-carboxylate

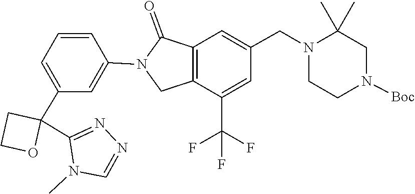 [SMILES: Cn1cnnc1C1(c2cccc(N3Cc4c(cc(CN5CCN(C(=O)OC(C)(C)C)CC5(C)C)cc4C(F)(F)F)C3=O)c2)CCO1]

To a solution of 3-[2-(3-bromophenyl)oxetan-2-yl]-4-methyl-1,2,4-triazole (410 mg, 1.4 mmol) and tert-butyl 3,3-dimethyl-4-[[3-oxo-7-(trifluoromethyl)isoindolin-5-yl]methyl]piperazine-1-carboxylate (623 mg, 1.4 mmol) in 1,4-dioxane (10 mL) were added XPhos Pd G3 (116 mg, 137 μmol) and K₃PO₄ (729 mg, 3.4 mmol). The mixture was stirred at 110° C. for 12 h under nitrogen atmosphere, then concentrated. The residue was purified by flash column chromatography (0-4% methanol in dichloromethane) to afford tert-butyl 3,3-dimethyl-4-[[2-[3-[2-(4-methyl-1,2,4-triazol-3-yl)oxetan-2-yl]phenyl]-3-oxo-7-(trifluoromethyl)isoindolin-5-yl]methyl]piperazine-1-carboxylate (400 mg, 44%) as a light yellow foam. MS (ESI) m/z: 641.4 [M+H]⁺; ¹H NMR (400 MHz, CDCl₃) δ 8.12 (s, 1H), 8.07 (s, 1H), 7.99 (br s, 1H), 7.87-7.80 (m, 2H), 7.46 (t, J=8.0 Hz, 1H), 7.14 (br d, J=7.2 Hz, 1H), 5.01 (s, 2H), 4.83-4.77 (m, 2H), 4.19-4.10 (m, 1H), 3.67 (br s, 2H), 3.38 (s, 5H), 3.29-3.24 (m, 2H), 3.01 (td, J=8.4, 11.2 Hz, 1H), 2.39 (br s, 2H), 1.47 (s, 9H), 1.14 (br s, 6H).

### Step 12: Preparation of 6-[(2,2-dimethylpiperazin-1-yl)methyl]-2-[3-[2-(4-methyl-1,2,4-triazol-3-yl)oxetan-2-yl]phenyl]-4-(trifluoromethyl)isoindolin-1-one

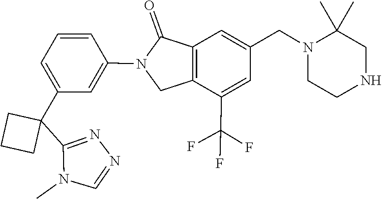 [SMILES: Cn1cnnc1C1(c2cccc(N3Cc4c(cc(CN5CCNCC5(C)C)cc4C(F)(F)F)C3=O)c2)CCC1]

To a solution of tert-butyl 3,3-dimethyl-4-[[2-[3-[2-(4-methyl-1,2,4-triazol-3-yl)oxetan-2-yl]phenyl]-3-oxo-7-(trifluoromethyl)isoindolin-5-yl]methyl]piperazine-1-carboxylate (100 mg, 0.15 mmol) in dichloromethane (3.0 mL) was added TFA (1 mL). The mixture was stirred at 25° C. for 1 h, then concentrated. The residue was diluted with acetonitrile and water, then lyophilized to afford 6-[(2,2-dimethylpiperazin-1-yl)methyl]-2-[3-[2-(4-methyl-1,2,4-triazol-3-yl)oxetan-2-yl]phenyl]-4-(trifluoromethyl)isoindolin-1-one (140 mg, crude, 3 TFA salt) as a yellow solid, which was used in the next step without further purification. MS (ESI) m/z: 541.4 [M+H]⁺.

### Step 13: Preparation of 6-(2,6-dibenzyloxy-3-pyridyl)-2-[4-[4-(dimethoxymethyl)cyclohexoxy]-1-piperidyl]-7-methyl-7H-pyrrolo[3,4-b]pyridin-5-one

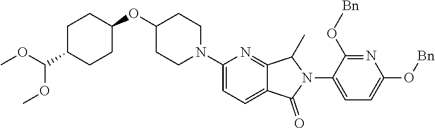

To a solution of 2-chloro-6-(2,6-dibenzyloxy-3-pyridyl)-7-methyl-7H-pyrrolo[3,4-b]pyridin-5-one (8.34 g, 18 mmol) in DMSO (42 mL) were added N,N-diisopropylethylamine (42 mL, 0.2 mol) and 4-[4-(dimethoxymethyl)cyclohexoxy]piperidine (5.46 g, 21 mmol). The mixture was stirred at 140° C. for 16 h, then diluted with water (80 mL) and extracted with ethyl acetate (80 mL×3). The combined organic phases were washed with brine (80 mL), dried over sodium sulfate, filtered, and concentrated under reduced pressure. The residue was purified by flash column chromatography (0-51% ethyl acetate in petroleum ether) to afford 6-(2,6-dibenzyloxy-3-pyridyl)-2-[4-[4-(dimethoxymethyl)cyclohexoxy]-1-piperidyl]-7-methyl-7H-pyrrolo[3,4-b]pyridin-5-one (10.2 g, 77%) as a red oil. MS (ESI) m/z: 693.4 [M+H]⁺.

### Step 14: Preparation of 3-[2-[4-[4-(dimethoxymethyl)cyclohexoxy]-1-piperidyl]-7-methyl-5-oxo-7H-pyrrolo[3,4-b]pyridin-6-yl]piperidine-2,6-dione

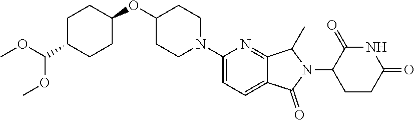 [SMILES: COC(OC)[C@H]1CC[C@H](OC2CCN(c3ccc4c(n3)C(C)N(C3CCC(=O)NC3=O)C4=O)CC2)CC1]

To a solution of 6-(2,6-dibenzyloxy-3-pyridyl)-2-[4-[4-(dimethoxymethyl)cyclohexoxy]-1-piperidyl]-7-methyl-7H-pyrrolo[3,4-b]pyridin-5-one (700 mg, 1 mmol) in tetrahydrofuran (20 mL) was added 20% Pd(OH)₂ on carbon (300 mg). The mixture was degassed under vacuum and purged with hydrogen, then stirred under hydrogen (15 psi) at 25° C. for 16 h. The mixture was filtered and washed with tetrahydrofuran (20 mL×3). The filtrate was concentrated under reduced pressure to afford 3-[2-[4-[4-(dimethoxymethyl)cyclohexoxy]-1-piperidyl]-7-methyl-5-oxo-7H-pyrrolo[3,4-b]pyridin-6-yl]piperidine-2,6-dione (432 mg, 73%, 87% purity) as a white foam, which was used in the next step without further purification. MS (ESI) m/z: 515.2 [M+H]⁺.

### Step 15: Preparation of 4-[[1-[6-(2,6-dioxo-3-piperidyl)-7-methyl-5-oxo-7H-pyrrolo[3,4-b]pyridin-2-yl]-4-piperidyl]oxy]cyclohexanecarbaldehyde

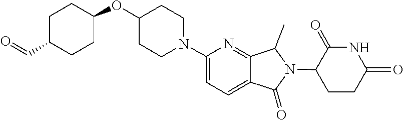 [SMILES: CC1c2nc(N3CCC(O[C@H]4CC[C@H](C=O)CC4)CC3)ccc2C(=O)N1C1CCC(=O)NC1=O]

To a solution of 3-[2-[4-[4-(dimethoxymethyl)cyclohexoxy]-1-piperidyl]-7-methyl-5-oxo-7H-pyrrolo[3,4-b]pyridin-6-yl]piperidine-2,6-dione (220 mg, 0.4 mmol) in water (2.0 mL) and acetone (4.0 mL) was added tosylic acid monohydrate (28.5 mg, 0.15 mmol). The mixture was stirred at 70° C. for 1 h. The pH of the mixture was adjusted to 7-8 by saturated sodium bicarbonate solution, and the resulting mixture was extracted with dichloromethane (20 mL×3). The combined organic layer was washed with brine (30 mL), dried over sodium sulfate, filtered, and concentrated. The residue was purified by flash column chromatography (0-4% methanol in dichloromethane) to afford 4-[[1-[6-(2,6-dioxo-3-piperidyl)-7-methyl-5-oxo-7H-pyrrolo[3,4-b]pyridin-2-yl]-4-piperidyl]oxy]cyclohexanecarbaldehyde (158 mg, 87%) as a white foam. MS (ESI) m/z: 469.2 [M+H]⁺.

### Step 16: Preparation of 3-[2-[4-[4-[[3,3-dimethyl-4-[[2-[3-[2-(4-methyl-1,2,4-triazol-3-yl)oxetan-2-yl]phenyl]-3-oxo-7-(trifluoromethyl)isoindolin-5-yl]methyl]piperazin-1-yl]methyl]cyclohexoxy]-1-piperidyl]-7-methyl-5-oxo-7H-pyrrolo[3,4-b]pyridin-6-yl]piperidine-2,6-dione

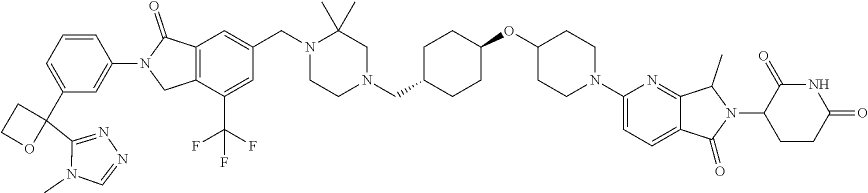 [SMILES: CC1c2nc(N3CCC(O[C@H]4CC[C@H](CN5CCN(Cc6cc7c(c(C(F)(F)F)c6)CN(c6cccc(C8(c9nncn9C)CCO8)c6)C7=O)C(C)(C)C5)CC4)CC3)ccc2C(=O)N1C1CCC(=O)NC1=O]

A mixture of 6-[(2,2-dimethylpiperazin-1-yl)methyl]-2-[3-[2-(4-methyl-1,2,4-triazol-3-yl)oxetan-2-yl]phenyl]-4-(trifluoromethyl)isoindolin-1-one (140 mg, 0.15 mmol, 3 TFA) and sodium acetate (71.8 mg, 0.9 mmol) in dichloromethane (2.0 mL) and DMSO (0.5 mL) was stirred for 10 min. 4-[[1-[6-(2,6-Dioxo-3-piperidyl)-7-methyl-5-oxo-7H-pyrrolo[3,4-b]pyridin-2-yl]-4-piperidyl]oxy]cyclohexanecarbaldehyde (78 mg, 0.2 mmol) in dichloromethane (2.0 mL) was then dropwise added at 0° C., and the mixture was stirred at 0° C. for 1 h before the addition of NaBH(OAc)₃ (77 mg, 0.4 mmol). The reaction mixture was stirred at 0° C. for 1 h. The mixture was diluted with saturated sodium bicarbonate solution (20 mL) and extracted with dichloromethane (20 mL×3). The combined organic layer was washed with brine (30 mL), dried over sodium sulfate, filtered, and concentrated. The residue was purified by prep-HPLC (46-86% acetonitrile in water (NH4HCO3) over 32 min at 60 mL/min) to afford 3-[2-[4-[4-[[3,3-dimethyl-4-[[2-[3-[2-(4-methyl-1,2,4-triazol-3-yl)oxetan-2-yl]phenyl]-3-oxo-7-(trifluoromethyl)isoindolin-5-yl]methyl]piperazin-1-yl]methyl]cyclohexoxy]-1-piperidyl]-7-methyl-5-oxo-7H-pyrrolo[3,4-b]pyridin-6-yl]piperidine-2,6-dione (51.8 mg, 36%) as a white solid. MS (ESI) m/z: 993.5 [M+H]⁺; ¹H NMR (400 MHz, DMSO-d₆) δ 10.88 (br d, J=12.8 Hz, 1H), 8.47 (s, 1H), 8.09 (s, 1H), 7.99 (s, 1H), 7.91 (s, 1H), 7.78 (br d, J=9.2 Hz, 1H), 7.69 (dd, J=2.8, 8.8 Hz, 1H), 7.50 (t, J=8.0 Hz, 1H), 7.08 (br d, J=7.6 Hz, 1H), 6.87 (br dd, J=3.2, 8.8 Hz, 1H), 5.19 (br s, 2H), 4.76-4.62 (m, 3H), 4.52-4.36 (m, 1H), 4.08 (br dd, J=14.4, 17.6 Hz, 2H), 3.97-3.86 (m, 1H), 3.76-3.49 (m, 3H), 3.45 (br d, J=7.6 Hz, 2H), 3.31 (br d, J=5.2 Hz, 3H), 3.28 (s, 3H), 2.94-2.85 (m, 1H), 2.83-2.70 (m, 1H), 2.61-2.52 (m, 2H), 2.35 (br s, 2H), 2.29-2.09 (m, 3H), 2.01-1.89 (m, 5H), 1.86-1.74 (m, 4H), 1.43-1.34 (m, 6H), 1.10 (s, 7H), 0.93-0.81 (m, 2H).

### Example 282: Exemplary synthesis of 3-[7-methyl-2-[4-[4-[[4-[[6-[3-[3-[(4-methyl-1,2,4-triazol-3-yl)methyl]oxetan-3-yl]phenyl]-7-oxo-4-(trifluoromethyl)-5H-pyrrolo[3,4-b]pyridin-2-yl]methyl]piperazin-1-yl]methyl]cyclohexoxy]-1-piperidyl]-5-oxo-7Hpyrrolo[3,4-b]pyridin-6-yl]piperidine-2,6-dione

### Step 1: Preparation of 2-chloro-3-methyl-4-(trifluoromethyl)pyridine

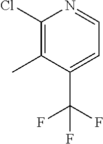

To a mixture of 2-chloro-4-(trifluoromethyl)pyridine (10 g, 55 mmol) in tetrahydrofuran (150 mL) was added LDA (2 M, 33 mL) at −78° C. The mixture was stirred at −78° C. for 1.5 h, then methyl iodide (9.37 g, 66 mmol) was added, and the mixture was stirred at −78° C. for 1 h. The reaction was quenched with saturated ammonium chloride solution (300 mL) at −78° C., and the resulting mixture was extracted with ethyl acetate (200 mL×3). The combined organic phase was washed with brine (200 mL), dried over anhydrous sodium sulfate, filtered, and concentrated. The residue was purified by flash column chromatography (7% ethyl acetate in petroleum ether) to afford 2-chloro-3-methyl-4-(trifluoromethyl)pyridine (7 g, 55%) as a yellow oil. MS (ESI) m/z: 196.0 [M+H]⁺; ¹H NMR (400 MHz, CDCl₃) δ 8.40 (d, J=4.8 Hz, 1H), 7.46 (d, J=4.8 Hz, 1H), 2.53 (s, 3H).

### Step 2: Preparation of methyl 3-methyl-4-(trifluoromethyl)pyridine-2-carboxylate

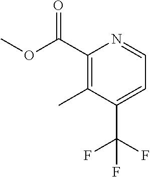

To a mixture of 2-chloro-3-methyl-4-(trifluoromethyl)pyridine (7.8 g, 40 mmol) and triethylamine (12.1 g, 119 mmol) in methanol (200 mL) was added Pd(dppf)Cl₂ (2.92 g, 4 mmol). The mixture was degassed under vacuum and purged with CO, then stirred under CO (40 psi) at 80° C. for 12 h. The mixture was concentrated under reduced pressure, and the residue was diluted with water (100 mL) and extracted with ethyl acetate (100 mL×3). The combined organic phase was washed with brine (100 mL), dried over anhydrous sodium sulfate, filtered, and concentrated. The residue was purified by flash column chromatography (16% ethyl acetate in petroleum ether) to afford methyl 3-methyl-4-(trifluoromethyl)pyridine-2-carboxylate (8.7 g, 85%) as a yellow oil. MS (ESI) m/z: 220.0 [M+H]⁺; ¹H NMR (400 MHz, CDCl₃) δ 8.67 (d, J=4.8 Hz, 1H), 7.64 (d, J=4.8 Hz, 1H), 4.01 (s, 3H), 2.63 (s, 3H).

### Step 3: Preparation of methyl 3-methyl-1-oxido-4-(trifluoromethyl)pyridin-1-ium-2-carboxylate

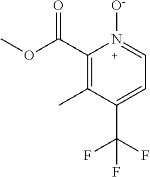

To a mixture of methyl 3-methyl-4-(trifluoromethyl)pyridine-2-carboxylate (4.0 g, 18 mmol) in dichloromethane (120 mL) was added trifluoroacetic anhydride (7.66 g, 36 mmol) and urea hydrogen peroxide (3.43 g, 36 mmol). The mixture was stirred at 20-25° C. for 16 h. The mixture was diluted with dichloromethane (50 mL) and washed with saturated sodium bicarbonate solution (50 mL×2), then saturated sodium sulfite solution (50 mL×2). The organic layer was dried over anhydrous sodium sulfate, filtered, and concentrated to afford methyl 3-methyl-1-oxido-4-(trifluoromethyl)pyridin-1-ium-2-carboxylate (3.1 g, 57%, 79% purity) as a yellow oil, which was used in the next step without further purification. MS (ESI) m/z: 236.0 [M+H]⁺.

### Step 4: Preparation of methyl 6-chloro-3-methyl-4-(trifluoromethyl)pyridine-2-carboxylate

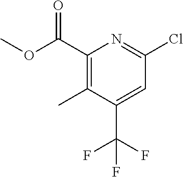 [SMILES: COC(=O)c1nc(Cl)cc(C(F)(F)F)c1C]

A mixture of methyl 3-methyl-1-oxido-4-(trifluoromethyl)pyridin-1-ium-2-carboxylate (3.1 g, 10 mmol) in phosphoryl chloride (30 mL) was stirred at 70° C. for 12 h. The mixture was cooled to 25° C., slowly poured into water (300 mL) and stirred for 30 min. Solid sodium bicarbonate was added to the above mixture to adjust the pH to 7-8, then the mixture was extracted with ethyl acetate (50 mL×3). The combined organic phase was washed with brine (50 mL), dried over anhydrous sodium sulfate, filtered, and concentrated. The residue was purified by flash column chromatography (12-13% ethyl acetate in petroleum ether) to afford methyl 6-chloro-3-methyl-4-(trifluoromethyl)pyridine-2-carboxylate (2.2 g, 79%) as a colorless oil. MS (ESI) m/z: 254.0 [M+H]⁺; ¹H NMR (400 MHz, CDCl₃) δ 7.67 (s, 1H), 4.00 (s, 3H), 2.57 (s, 3H).

### Step 5: Preparation of methyl 3-(bromomethyl)-6-chloro-4-(trifluoromethyl)pyridine-2-carboxylate

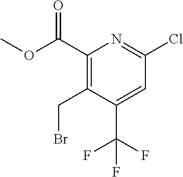 [SMILES: COC(=O)c1nc(Cl)cc(C(F)(F)F)c1CBr]

A mixture of methyl 6-chloro-3-methyl-4-(trifluoromethyl)pyridine-2-carboxylate (2.2 g, 9 mmol) and NBS (3.09 g, 17 mmol) in acetonitrile (30 mL) was illuminated under 440 nm blue light in the flow reactor (1.5 mL/min) for 20 min. The mixture was concentrated under reduced pressure. The residue was purified by flash column chromatography (12-13% ethyl acetate in petroleum ether) to afford methyl 3-(bromomethyl)-6-chloro-4-(trifluoromethyl)pyridine-2-carboxylate (2.4 g, 76%) as a colorless oil. MS (ESI) m/z: 333.9 [M+H]⁺; ¹H NMR (400 MHz, CDCl₃) δ 7.73 (s, 1H), 4.95 (s, 2H), 4.05 (s, 3H).

### Step 6: Preparation of 2-chloro-6-[3-[3-[(4-methyl-1,2,4-triazol-3-yl)methyl]oxetan-3-yl]phenyl]-4-(trifluoromethyl)-5Hpyrrolo[3,4-b]pyridin-7-one

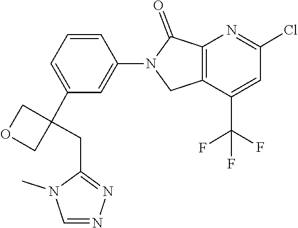 [SMILES: Cn1cnnc1CC1(c2cccc(N3Cc4c(C(F)(F)F)cc(Cl)nc4C3=O)c2)COC1]

To a mixture of 3-[3-[(4-methyl-1,2,4-triazol-3-yl)methyl]oxetan-3-yl]aniline (400 mg, 1.6 mmol) and methyl 3-(bromomethyl)-6-chloro-4-(trifluoromethyl)pyridine-2-carboxylate (541 mg, 1.6 mmol) in acetonitrile (10 mL) and water (5.0 mL) were added (2,2,2-trifluoroacetyl)oxysilver (540 mg, 2.4 mmol) and TFA (240 μL, 3.2 mmol). The mixture was stirred at 25° C. for 12 h then 50° C. for 12 h. The pH of the mixture was adjusted to 8-9 with saturated sodium bicarbonate solution, then the mixture was filtered, and the filtrate was extracted with ethyl acetate (30 mL×3). The combined organic layer was washed with brine (20 mL), dried over sodium sulfate, filtered, and concentrated. The residue was purified by flash column chromatography (6% methanol in dichloromethane) to afford 2-chloro-6-[3-[3-[(4-methyl-1,2,4-triazol-3-yl)methyl]oxetan-3-yl]phenyl]-4-(trifluoromethyl)-5Hpyrrolo[3,4-b]pyridin-7-one (150 mg, 19%) as a yellow solid. MS (ESI) m/z: 464.1 [M+H]⁺; ¹H NMR (400 MHz, CDCl₃) δ 8.30 (s, 1H), 8.19 (s, 1H), 7.92 (dd, J=1.6, 8.0 Hz, 1H), 7.45 (s, 1H), 7.38 (t, J=8.0 Hz, 1H), 6.83 (d, J=8.0 Hz, 1H), 5.14 (s, 2H), 4.98 (d, J=6.0 Hz, 2H), 4.90 (d, J=6.0 Hz, 2H), 3.53 (s, 2H), 2.94 (s, 3H).

### Step 7: Preparation of tert-butyl 4-[[6-[3-[3-[(4-methyl-1,2,4-triazol-3-yl)methyl]oxetan-3-yl]phenyl]-7-oxo-4-(trifluoromethyl)-5H-pyrrolo[3,4-b]pyridin-2-yl]methyl]piperazine-1-carboxylate

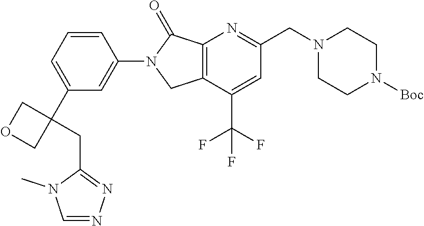 [SMILES: Cn1cnnc1CC1(c2cccc(N3Cc4c(C(F)(F)F)cc(CN5CCN(C(=O)OC(C)(C)C)CC5)nc4C3=O)c2)COC1]

To a mixture of 2-chloro-6-[3-[3-[(4-methyl-1,2,4-triazol-3-yl)methyl]oxetan-3-yl]phenyl]-4-(trifluoromethyl)-5H-pyrrolo[3,4-b]pyridin-7-one (150 mg, 0.3 mmol) and potassium (4-tert-butoxycarbonylpiperazin-1-yl)methyl-trifluoro-boranuide (195 mg, 0.6 mmol) in 1,4-dioxane (5.0 mL) and water (1.0 mL) were added Pd(OAc)₂ (15 mg, 67 μmol), cesium carbonate (318 mg, 1 mmol) and bis(1-adamantyl)-butyl-phosphane (45 mg, 0.1 mmol). The mixture was stirred at 110° C. for 12 h under nitrogen atmosphere. The mixture was cooled to 25° C. and poured into water (50 mL). The aqueous mixture was extracted with ethyl acetate (50 mL×3). The combined organic phase was washed with brine (50 mL), dried over anhydrous sodium sulfate, filtered, and concentrated. The residue was purified by flash column chromatography (8-10% methanol in dichloromethane) to afford tert-butyl 4-[[6-[3-[3-[(4-methyl-1,2,4-triazol-3-yl)methyl]oxetan-3-yl]phenyl]-7-oxo-4-(trifluoromethyl)-5H-pyrrolo[3,4-b]pyridin-2-yl]methyl]piperazine1-carboxylate (160 mg, 56% purity, 44%) as a yellow solid. MS (ESI) m/z: 650.2 [M+Na]⁺.

### Step 8: Preparation of 2-[3-[1-[(4-methyl-1,2,4-triazol-3-yl)methyl]cyclobutyl]phenyl]-4-(trifluoromethyl)-6-vinyl-isoindolin-1-one

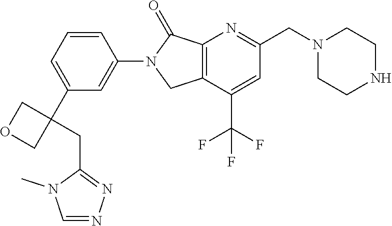 [SMILES: Cn1cnnc1CC1(c2cccc(N3Cc4c(C(F)(F)F)cc(CN5CCNCC5)nc4C3=O)c2)COC1]

To a solution of tert-butyl 4-[[6-[3-[3-[(4-methyl-1,2,4-triazol-3-yl)methyl]oxetan-3-yl]phenyl]-7-oxo-4-(trifluoromethyl)-5H-pyrrolo[3,4-b]pyridin-2-yl]methyl]piperazine-1-carboxylate (160 mg, 0.3 mmol, 56% purity) in dichloromethane (1.0 mL) was added TFA (300 uL, 4 mmol). The reaction solution was stirred at 25° C. for 1 h, then concentrated under reduced pressure and lyophilized to afford 6-[3-[3-[(4-methyl-1,2,4-triazol-3-yl)methyl]oxetan-3-yl]phenyl]-2-(piperazin-1-ylmethyl)-4-(trifluoromethyl)-5H-pyrrolo[3,4-b]pyridin-7-one (240 mg, crude, 4 TFA salt) as a yellow solid. MS (ESI) m/z: 528.2 [M+H]⁺.

### Step 9: Preparation of 3-[7-methyl-2-[4-[4-[[4-[[6-[3-[3-[(4-methyl-1,2,4-triazol-3-yl)methyl]oxetan-3-yl]phenyl]-7-oxo-4-(trifluoromethyl)-5H-pyrrolo[3,4-b]pyridin-2-yl]methyl]piperazin-1-yl]methyl]cyclohexoxy]-1-piperidyl]-5-oxo-7H-pyrrolo[3,4-b]pyridin-6-yl]piperidine-2,6-dione

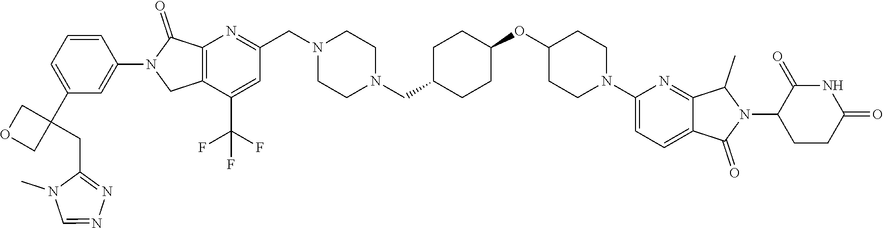 [SMILES: CC1c2nc(N3CCC(O[C@H]4CC[C@H](CN5CCN(Cc6cc(C(F)(F)F)c7c(n6)C(=O)N(c6cccc(C8(Cc9nncn9C)COC8)c6)C7)CC5)CC4)CC3)ccc2C(=O)N1C1CCC(=O)NC1=O]

To a mixture of 6-[3-[3-[(4-methyl-1,2,4-triazol-3-yl)methyl]oxetan-3-yl]phenyl]-2-(piperazin-1-ylmethyl)-4-(trifluoromethyl)-5H-pyrrolo[3,4-b]pyridin-7-one (240 mg, 0.24 mmol, 4 TFA) in dichloromethane (3.0 mL) and DMSO (0.6 mL) was added sodium acetate (120 mg, 1.5 mmol) at 0° C. The mixture was stirred at 0° C. for 15 min, then 4-[[1-[6-(2,6-dioxo-3-piperidyl)-7-methyl-5-oxo-7H-pyrrolo[3,4-b]pyridin-2-yl]-4-piperidyl]oxy]cyclohexanecarbaldehyde (137 mg, 0.3 mmol) in dichloromethane (3.0 mL) was added at 0° C. The mixture was stirred at 0° C. for 1 h before the addition of NaBH(OAc)₃ (155 mg, 0.7 mmol). The reaction mixture was stirred at 0° C. for 1 h. The mixture was diluted with saturated sodium bicarbonate solution (10 mL) and extracted with dichloromethane (20 mL×3). The combined organic phase was dried over anhydrous sodium sulfate, filtered, and concentrated. The residue was purified by prep-HPLC (16%-56% acetonitrile in water (NH₄HCO₃) over 25 min at 60 mL/min) to afford 3-[7-methyl-2-[4-[4-[[4-[[6-[3-[3-[(4-methyl-1,2,4-triazol-3-yl)methyl]oxetan-3-yl]phenyl]-7-oxo-4-(trifluoromethyl)-5H-pyrrolo[3,4-b]pyridin-2-yl]methyl]piperazin-1-yl]methyl]cyclohexoxy]-1-piperidyl]-5-oxo-7H-pyrrolo[3,4-b]pyridin-6-yl]piperidine-2,6-dione (38 mg, 16%) as a white solid. MS (ESI) m/z: 490.9 [M/2+H]⁺; ¹H NMR (400 MHz, DMSO-d₆) δ 10.89 (br d, J=12.8 Hz, 1H), 8.19 (s, 1H), 8.01-7.89 (m, 2H), 7.69 (dd, J=3.2, 8.8 Hz, 1H), 7.54-7.33 (m, 2H), 6.88 (dd, J=3.2, 8.8 Hz, 1H), 6.82 (d, J=7.6 Hz, 1H), 5.11 (s, 2H), 4.98 (d, J=6.0 Hz, 2H), 4.90 (d, J=6.0 Hz, 2H), 4.69 (dd, J=4.8, 12.0 Hz, 1H), 4.56-4.37 (m, 1H), 4.16-4.01 (m, 2H), 3.82 (s, 2H), 3.76-3.64 (m, 1H), 3.53 (s, 2H), 2.92 (s, 3H), 2.71-2.56 (m, 8H), 2.42-2.30 (m, 4H), 2.07 (br d, J=6.8 Hz, 2H), 2.01-1.72 (m, 8H), 1.47-1.35 (m, 6H), 1.22-1.06 (m, 3H), 0.97-0.74 (m, 2H).

### Example 283: Exemplary synthesis of 3-[3-[6-[4-[[7-[[1-[1-(2,6-dioxo-3-piperidyl)-3-isopropyl-2-oxo-benzimidazol-4-yl]-4-piperidyl]methyl]-2,7-diazaspiro[3.5]nonan-2-yl]methyl]phenyl]-1-oxo-4-(trifluoromethyl)isoindolin-2-yl]phenyl]-3-[(4-methyl-1,2,4-triazol-3-yl)methyl]cyclobutanecarbonitrile Step 1: Preparation of 3-[3-[6-(4-formylphenyl)-1-oxo-4-(trifluoromethyl)isoindolin-2-yl]phenyl]-3-[(4-methyl-1,2,4-triazol-3-yl)methyl]cyclobutanecarbonitrile

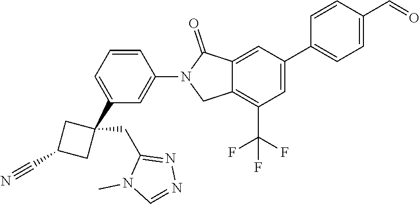 [SMILES: Cn1cnnc1C[C@]1(c2cccc(N3Cc4c(cc(-c5ccc(C=O)cc5)cc4C(F)(F)F)C3=O)c2)C[C@H](C#N)C1]

To a solution of 3-[3-[6-bromo-1-oxo-4-(trifluoromethyl)isoindolin-2-yl]phenyl]-3-[(4-methyl-1,2,4-triazol-3-yl)methyl]cyclobutanecarbonitrile (500 mg, 0.9 mmol) and (4-formylphenyl)boronic acid (212 mg, 1.4 mmol) in dioxane (10 mL) and water (1 mL) were added sodium carbonate (300 mg, 2.8 mmol) and [1,1′-bis(diphenylphosphino)ferrocene]dichloropalladium(II) (69 mg, 0.09 mmol). The mixture was stirred at 90° C. for 10 h under nitrogen atmosphere, then concentrated under reduced pressure. The residue was purified by silica gel column chromatography (petroleum ether/ethyl acetate=1/1 then dichloromethane/methanol=30/1 to 10/1) to afford 3-[3-[6-(4-formylphenyl)-1-oxo-4-(trifluoromethyl)isoindolin-2-yl]phenyl]-3-[(4-methyl-1,2,4-triazol-3-yl)methyl]cyclobutanecarbonitrile (390 mg, 74%) as a yellow solid. MS (ESI) m/z: 556.2 [M+H]⁺; ¹H NMR (400 MHz, DMSO-d₆) δ 10.14 (s, 1H), 8.36 (s, 1H), 8.13 (s, 1H), 8.07 (d, J=8.4 Hz, 2H), 7.84-7.90 (m, 3H), 7.71 (t, J=2.0 Hz, 1H), 7.57 (dd, J=8.4, 1.6 Hz, 1H), 7.38 (t, J=8.0 Hz, 1H), 6.85 (d, J=8.4 Hz, 1H), 5.03 (s, 2H), 3.39 (s, 2H), 3.07-3.17 (m, 3H), 3.00-3.06 (m, 2H), 2.88 (s, 3H).

### Step 2: Preparation of 3-[4-(dimethoxymethyl)-1-piperidyl]-2-nitro-aniline

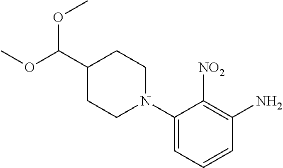 [SMILES: COC(OC)C1CCN(c2cccc(N)c2[N+](=O)[O-])CC1]

To a solution of 3-fluoro-2-nitro-aniline (10 g, 64 mmol) and 4-(dimethoxymethyl)piperidine (13.26 g, 83 mmol) in N,N-dimethylformamide (100 mL) was added cesium carbonate (62.61 g, 192 mmol). The reaction mixture was stirred at 25° C. for 10 h, then diluted with water (300 mL) and extracted with ethyl acetate (300 mL×3). The combined organic phase was washed with brine (300 mL), dried over anhydrous sodium sulfate, filtered, and concentrated under reduced pressure. The residue was purified by silica gel column chromatography (petroleum ether/ethyl acetate=1/0 to 30/1) to afford 3-[4-(dimethoxymethyl)-1-piperidyl]-2-nitro-aniline (14.92 g, 79%) as a red solid. MS (ESI) m/z: 296.2 [M+H]⁺; ¹H NMR (400 MHz, DMSO-d₆) δ 7.10 (t, J=8.0 Hz, 1H), 6.51 (dd, J=0.8, 8.4 Hz, 1H), 6.35 (dd, J=0.8, 8.0 Hz, 1H), 5.83 (s, 2H), 4.08 (d, J=6.4 Hz, 1H), 3.27 (s, 6H), 3.07 (d, J=12.0 Hz, 2H), 2.62 (t, J=11.2 Hz, 2H), 1.72-1.59 (m, 3H), 1.34-1.19 (m, 2H).

### Step 3: Preparation of 2,6-dibenzyloxy-N-[3-[4-(dimethoxymethyl)-1-piperidyl]-2-nitro-phenyl]pyridin-3-amine

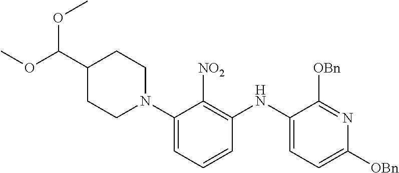 [SMILES: COC(OC)C1CCN(c2cccc(Nc3ccc(OCc4ccccc4)nc3OCc3ccccc3)c2[N+](=O)[O-])CC1]

A mixture of 3-[4-(dimethoxymethyl)-1-piperidyl]-2-nitro-aniline (3.20 g, 11 mmol), 2,6-dibenzyloxy-3-bromo-pyridine (4.41 g, 12 mmol), cesium carbonate (10.59 g, 32 mmol) and RuPhos Pd G3 (2.72 g, 3.2 mmol) in dioxane (1.5 mL) was degassed and purged with nitrogen, then the mixture was stirred at 110° C. for 10 h under nitrogen atmosphere. The reaction mixture was filtered and concentrated under reduced pressure. The residue was purified by silica gel column chromatography (petroleum ether/ethyl acetate=30/1 to 3/1) to afford 2,6-dibenzyloxy-N-[3-[4-(dimethoxymethyl)-1-piperidyl]-2-nitro-phenyl]pyridin-3-amine (5.20 g, 82%) as a red solid. MS (ESI) m/z: 585.4 [M+H]⁺; ¹H NMR (400 MHz, DMSO-d₆) δ 7.53 (d, J=8.4 Hz, 1H), 7.45-7.27 (m, 10H), 7.18 (t, J=8.4 Hz, 1H), 6.66 (d, J=7.6 Hz, 1H), 6.46 (d, J=8.0 Hz, 1H), 6.32 (d, J=8.4 Hz, 1H), 5.33 (d, J=13.2 Hz, 4H), 4.10 (d, J=6.4 Hz, 1H), 3.28 (s, 6H), 3.15-3.07 (m, 2H), 2.69 (t, J=11.2 Hz, 2H), 1.68 (d, J=9.6 Hz, 3H), 1.40-1.19 (m, 3H).

### Step 4: Preparation of N1-(2,6-dibenzyloxy-3-pyridyl)-3-[4-(dimethoxymethyl)-1-piperidyl]benzene-1,2-diamine

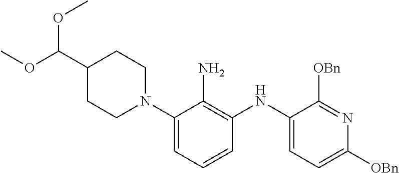 [SMILES: COC(OC)C1CCN(c2cccc(Nc3ccc(OCc4ccccc4)nc3OCc3ccccc3)c2N)CC1]

To a solution of 2,6-dibenzyloxy-N-[3-[4-(dimethoxymethyl)-1-piperidyl]-2-nitro-phenyl]pyridin-3-amine (5.20 g, 9 mmol) in ethanol (60 mL) and water (6 mL) were slowly added iron (2.48 g, 44 mmol), acetic acid (0.05 g, 0.9 mol) and ammonium chloride (2.38 g, 44 mmol). The mixture was stirred at 50° C. for 20 h. The reaction mixture was filtered and concentrated under reduced pressure. The residue was purified by silica gel column chromatography (petroleum ether/ethyl acetate=20/1 to 3/1) to afford N1-(2,6-dibenzyloxy-3-pyridyl)-3-[4-(dimethoxymethyl)-1-piperidyl]benzene-1,2-diamine (3.60 g, 73%) as a red solid.

### Step 5: Preparation of 3-(2,6-dibenzyloxy-3-pyridyl)-7-[4-(dimethoxymethyl)-1-piperidyl]-1H-benzimidazol-2-one

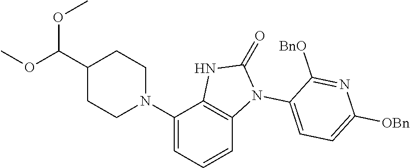 [SMILES: COC(OC)C1CCN(c2cccc3c2[nH]c(=O)n3-c2ccc(OCc3ccccc3)nc2OCc2ccccc2)CC1]

To a solution of Ni-(2,6-dibenzyloxy-3-pyridyl)-3-[4-(dimethoxymethyl)-1-piperidyl]benzene-1,2-diamine (3.57 g, 6 mmol) in tetrahydrofuran (40 mL) were added di(imidazol-1-yl)methanone (2.09 g, 13 mmol) and 4-dimethylaminopyridine (0.08 mg, 0.6 mmol) at 0° C. The mixture was stirred at 25° C. for 10 h, then concentrated under reduced pressure. The residue was purified by silica gel column chromatography (petroleum ether/ethyl acetate=10/1 to 1/1) to afford 3-(2,6-dibenzyloxy-3-pyridyl)-7-[4-(dimethoxymethyl)-1-piperidyl]-1H-benzimidazol-2-one (3.61 g, 97%) as a red oil. MS (ESI) m/z: 581.4 [M+H]⁺; ¹H NMR (400 MHz, DMSO-d₆) δ 10.99 (s, 1H), 7.79 (d, J=8.4 Hz, 1H), 7.48-7.43 (m, 2H), 7.42-7.33 (m, 3H), 7.27 (s, 5H), 6.90-6.83 (m, 1H), 6.67 (d, J=8.0 Hz, 1H), 6.60 (d, J=8.4 Hz, 1H), 6.29 (d, J=7.6 Hz, 1H), 5.44-5.33 (m, 4H), 4.12 (d, J=6.0 Hz, 1H), 3.31-3.27 (m, 8H), 2.63-2.53 (m, 2H), 1.79-1.63 (m, 3H), 1.62-1.48 (m, 2H).

### Step 6: Preparation of 1-(2,6-dibenzyloxy-3-pyridyl)-4-[4-(dimethoxymethyl)-1-piperidyl]-3-isopropyl-benzimidazol-2-one

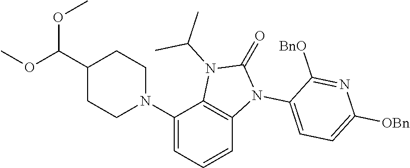 [SMILES: COC(OC)C1CCN(c2cccc3c2n(C(C)C)c(=O)n3-c2ccc(OCc3ccccc3)nc2OCc2ccccc2)CC1]

To a solution of 3-(2,6-dibenzyloxy-3-pyridyl)-7-[4-(dimethoxymethyl)-1-piperidyl]-1H-benzimidazol-2-one (3.61 g, 6 mmol) and 2-iodopropane (1.9 mL, 19 mmol) in N,N-dimethylformamide (40 mL) was added cesium carbonate (6.08 g, 19 mmol). The mixture was stirred at 50° C. for 10 h, then poured into ice-water. The resulting suspension was filtered, and the filter cake was washed with methanol (3 mL×2) to afford 1-(2,6-dibenzyloxy-3-pyridyl)-4-[4-(dimethoxymethyl)-1-piperidyl]-3-isopropyl-benzimidazol-2-one (3.60 g, 93%) as a white solid, which was used in the next step without further purification. MS (ESI) m/z: 623.4 [M+H]⁺; ¹H NMR (400 MHz, DMSO-d₆) δ 7.81 (d, J=8.4 Hz, 1H), 7.50-7.44 (m, 2H), 7.42-7.32 (m, 3H), 7.26 (s, 5H), 6.95-6.85 (m, 2H), 6.61 (d, J=8.4 Hz, 1H), 6.39 (dd, J=2.8, 6.0 Hz, 1H), 5.70-5.55 (m, 1H), 5.45-5.28 (m, 4H), 4.15 (d, J=6.8 Hz, 1H), 3.30 (s, 6H), 3.18-3.07 (m, 2H), 2.79-2.61 (m, 2H), 2.60-2.52 (m, 1H), 1.78 (d, J=13.2 Hz, 2H), 1.74-1.63 (m, 1H), 1.53 (d, J=6.8 Hz, 3H), 1.47 (d, J=6.8 Hz, 3H), 1.44-1.33 (m, 2H).

### Step 7: Preparation of 3-[4-[4-(dimethoxymethyl)-1-piperidyl]-3-isopropyl-2-oxo-benzimidazol-1-yl]piperidine-2,6-dione

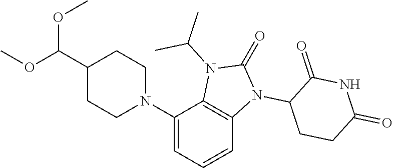 [SMILES: COC(OC)C1CCN(c2cccc3c2n(C(C)C)c(=O)n3C2CCC(=O)NC2=O)CC1]

To a solution of 1-(2,6-dibenzyloxy-3-pyridyl)-4-[4-(dimethoxymethyl)-1-piperidyl]-3-isopropyl-benzimidazol-2-one (2.50 g, 4 mmol) in tetrahydrofuran (25 mL) was added 10% palladium on carbon (500 mg). The suspension was degassed under vacuum and purged with hydrogen, then stirred under hydrogen (50 psi) at 50° C. for 10 h. The reaction mixture was filtered, and concentrated under reduced pressure to afford 3-[4-[4-(dimethoxymethyl)-1-piperidyl]-3-isopropyl-2-oxo-benzimidazol-1-yl]piperidine-2,6-dione (1.70 g, crude) as an off-white solid, which was used in the next step without further purification. MS (ESI) m/z: 445.1 [M+H]⁺.

### Step 8: Preparation of 1-[1-(2,6-dioxo-3-piperidyl)-3-isopropyl-2-oxo-benzimidazol-4-yl]piperidine-4-carbaldehyde

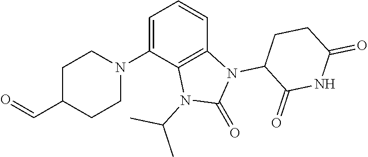 [SMILES: CC(C)n1c(=O)n(C2CCC(=O)NC2=O)c2cccc(N3CCC(C=O)CC3)c21]

To a solution of 3-[4-[4-(dimethoxymethyl)-1-piperidyl]-3-isopropyl-2-oxo-benzimidazol-1-yl]piperidine-2,6-dione (200 mg, 0.5 mmol) in dichloromethane (1.5 mL) was added trifluoroacetic acid (768 mg, 6.7 mmol). The reaction solution was stirred at 0° C. for 0.5 h, then concentrated under reduced pressure to afford 1-[1-(2,6-dioxo-3-piperidyl)-3-isopropyl-2-oxo-benzimidazol-4-yl]piperidine-4-carbaldehyde (178 mg, crude, TFA salt) as a yellow oil, which was used in the next step without further purification. MS (ESI) m/z: 399.2 [M+H]⁺.

### Step 9: Preparation of tert-butyl 7-[[1-[1-(2,6-dioxo-3-piperidyl)-3-isopropyl-2-oxo-benzimidazol-4-yl]-4-piperidyl]methyl]-2,7-diazaspiro[3.5]nonane-2-carboxylate

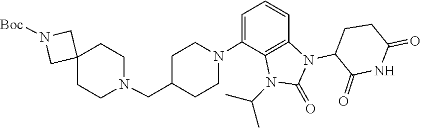 [SMILES: CC(C)n1c(=O)n(C2CCC(=O)NC2=O)c2cccc(N3CCC(CN4CCC5(CC4)CN(C(=O)OC(C)(C)C)C5)CC3)c21]

To a solution of 1-[1-(2,6-dioxo-3-piperidyl)-3-isopropyl-2-oxo-benzimidazol-4-yl]piperidine-4-carbaldehyde (178 mg, 0.5 mmol) in dichloromethane (5 mL) were added triethylamine (136 mg, 1.3 mmol) and tert-butyl 2,7-diazaspiro[3.5]nonane-2-carboxylate (152 mg, 0.7 mmol). The mixture was stirred at 30° C. for 2 h before the addition of sodium triacetoxyborohydride (284 mg, 1.3 mmol). The reaction mixture was stirred at 30° C. for 10 h, then concentrated under reduced pressure. The residue was purified by silica gel column chromatography (petroleum ether/ethyl acetate=10:1 to 0:1) to afford tert-butyl 7-[[1-[1-(2,6-dioxo-3-piperidyl)-3-isopropyl-2-oxo-benzimidazol-4-yl]-4-piperidyl]methyl]-2,7-diazaspiro[3.5]nonane-2-carboxylate (230 mg, 85%) as a white solid. MS (ESI) m/z: 609.4 [M+H]⁺; ¹H NMR (400 MHz, DMSO-d₆) δ 11.09 (s, 1H), 8.87-9.12 (m, 1H), 6.95-7.05 (m, 1H), 6.81-6.93 (m, 2H), 6.57 (s, 1H), 5.59 (m, 1H), 5.29 (m, 1H), 3.53-3.71 (m, 4H), 3.44-3.50 (m, 2H), 3.07-3.16 (m, 3H), 2.99-3.05 (m, 1H), 2.82-2.97 (m, 3H), 2.62-2.77 (m, 4H), 1.92-2.04 (m, 3H), 1.85 (d, J=12.0 Hz, 4H), 1.47 (m, 6H), 1.39 (s, 9H), 1.33 (s, 1H).

### Step 10: Preparation of 3-[4-[4-(2,7-diazaspiro[3.5]nonan-7-ylmethyl)-1-piperidyl]-3-isopropyl-2-oxo-benzimidazol-1-yl]piperidine-2,6-dione

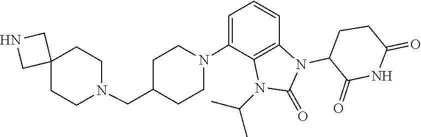 [SMILES: CC(C)n1c(=O)n(C2CCC(=O)NC2=O)c2cccc(N3CCC(CN4CCC5(CC4)CNC5)CC3)c21]

To a solution of tert-butyl 7-[[1-[1-(2,6-dioxo-3-piperidyl)-3-isopropyl-2-oxo-benzimidazol-4-yl]-4-piperidyl]methyl]-2,7-diazaspiro[3.5]nonane-2-carboxylate (100 mg, 0.2 mmol) in dichloromethane (3 mL) was added trifluoroacetic acid (1.54 g, 13 mmol). The reaction solution was stirred at 25° C. for 0.5 h, then concentrated under reduced pressure to afford 3-[4-[4-(2,7-diazaspiro[3.5]nonan-7-ylmethyl)-1-piperidyl]-3-isopropyl-2-oxo-benzimidazol-1-yl]piperidine-2,6-dione (100 mg, crude, TFA salt) as a yellow oil, which was used in the next step without further purification. MS (ESI) m/z: 509.4 [M+H]⁺.

### Step 11: Preparation of 3-[3-[6-[4-[[7-[[1-[1-(2,6-dioxo-3-piperidyl)-3-isopropyl-2-oxo-benzimidazol-4-yl]-4-piperidyl]methyl]-2,7-diazaspiro[3.5]nonan-2-yl]methyl]phenyl]-1-oxo-4-(trifluoromethyl)isoindolin-2-yl]phenyl]-3-[(4-methyl-1,2,4-triazol-3-yl)methyl]cyclobutanecarbonitrile

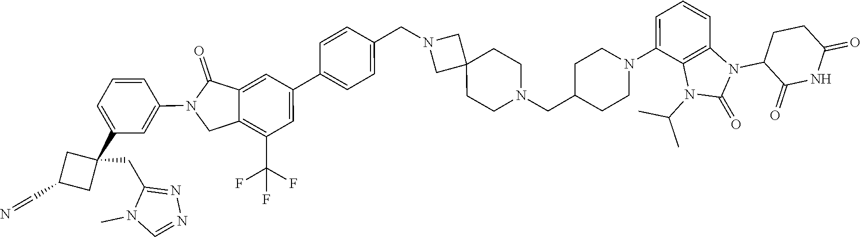

To a solution of 3-[4-[4-(2,7-diazaspiro[3.5]nonan-7-ylmethyl)-1-piperidyl]-3-isopropyl-2-oxo-benzimidazol-1-yl]piperidine-2,6-dione trifluoroacetate (94 mg, 0.2 mmol) in dichloromethane (3 mL) were added triethylamine (38 mg, 0.4 mmol), 3-[3-[6-(4-formylphenyl)-1-oxo-4-(trifluoromethyl)isoindolin-2-yl]phenyl]-3-[(4-methyl-1,2,4-triazol-3-yl)methyl]cyclobutanecarbonitrile (70 mg, 0.1 mmol) and tetraisopropoxytitanium (36 mg, 0.1 mmol). The mixture was stirred at 30° C. for 10 h before the addition of sodium triacetoxyborohydride (80 mg, 0.4 mmol). The reaction mixture was stirred at 30° C. for 1 h, then diluted with water (20 mL) and extracted with dichloromethane (20 mL). The organic layer was washed with brine (30 mL), dried over anhydrous sodium sulfate, filtered, and concentrated under reduced pressure. The residue was purified by prep-HPLC (8%-38% acetonitrile in water (formic acid) over 15 min) to afford 3-[3-[6-[4-[[7-[[1-[1-(2,6-dioxo-3-piperidyl)-3-isopropyl-2-oxo-benzimidazol-4-yl]-4-piperidyl]methyl]-2,7-diazaspiro[3.5]nonan-2-yl]methyl]phenyl]-1-oxo-4-(trifluoromethyl)isoindolin-2-yl]phenyl]-3-[(4-methyl-1,2,4-triazol-3-yl)methyl]cyclobutanecarbonitrile (44.6 mg, 34%) as a white solid. MS (ESI) m/z: 1048.6 [M+H]⁺; ¹H NMR (400 MHz, DMSO-d₆) δ 11.09 (s, 1H), 8.27 (d, J=4.4 Hz, 2H), 8.19 (s, 1H), 7.82-7.92 (m, 3H), 7.44-7.53 (m, 3H), 7.37 (t, J=8.0 Hz, 1H), 6.93-7.02 (m, 1H), 6.80-6.91 (m, 3H), 5.54-5.68 (m, 1H), 5.30 (m, 1H), 5.18 (s, 2H), 3.84 (s, 2H), 3.32 (s, 2H), 3.29-3.31 (m, 2H), 3.23-3.29 (m, 3H), 3.20 (d, J=11.6 Hz, 3H), 3.10 (d, J=11.6 Hz, 2H), 2.90 (d, J=8.0 Hz, 4H), 2.81 (s, 3H), 2.61-2.74 (m, 4H), 2.45-2.49 (m, 1H), 2.37-2.45 (m, 2H), 2.29 (d, J=11.2 Hz, 2H), 1.94-2.00 (m, 1H), 1.82 (d, J=12.0 Hz, 2H), 1.76 (s, 3H), 1.63-1.71 (m, 1H), 1.46 (m, 6H), 1.18-1.30 (m, 2H).

The following compounds in Table 1 were prepared according to the procedures described using the appropriate starting materials and intermediates.

TABLE 1

| | | | Analogous |
|---|---|---|---|
| | | | schematic |
| | | | or |
| Ex. | | | procedure |
| No. | Structure | IUPAC Name | reference |
| 23 | 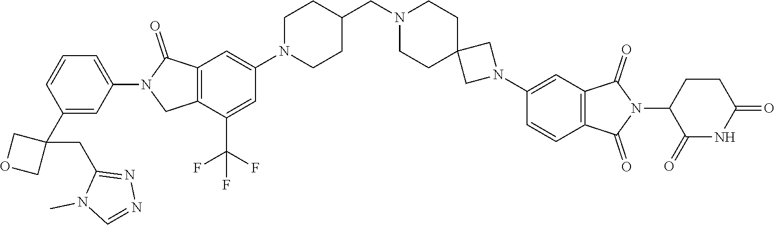 [SMILES: Cn1cnnc1CC1(c2cccc(N3Cc4c(cc(N5CCC(CN6CCC7(CC6)CN(c6ccc8c(c6)C(=O)N(C6CCC(=O)NC6=O)C8=O)C7)CC5)cc4C(F)(F)F)C3=O)c2)COC1] | 2-(2,6-dioxopiperidin-3-yl)-5-[7-({1- [2-(3-{3-[(4-methyl-4H-1,2,4-triazol-3- yl)methyl]oxetan-3-yl}phenyl)-3-oxo- 7-(trifluoromethyl)-2,3-dihydro-1H- isoindol-5-yl]piperidin-4-yl}methyl)- 2,7-diazaspiro[3.5]nonan-2-yl]-2,3- dihydro-1H-isoindole-1,3-dione | Example 11 |
| 24 | 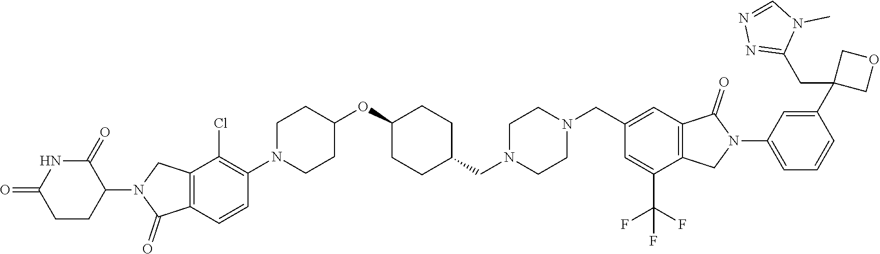 [SMILES: Cn1cnnc1CC1(c2cccc(N3Cc4c(cc(CN5CCN(C[C@H]6CC[C@H](OC7CCN(c8ccc9c(c8Cl)CN(C8CCC(=O)NC8=O)C9=O)CC7)CC6)CC5)cc4C(F)(F)F)C3=O)c2)COC1] | 3-[4-chloro-1-oxo-5-(4-{[(1r,4r)-4-[(4- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}piperazin-1- yl)methyl]cyclohexyl]oxy}piperidin-1- yl)-2,3-dihydro-1H-isoindol-2- yl]piperidine-2,6-dione | Example 22 |
| 25 | 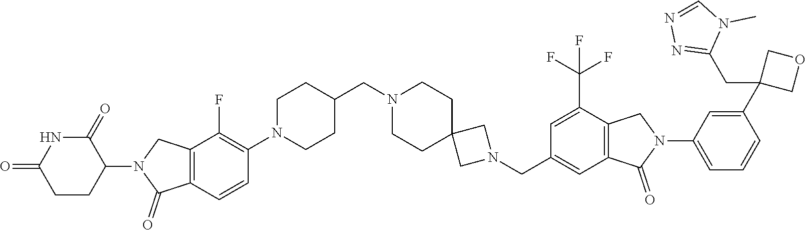 [SMILES: Cn1cnnc1CC1(c2cccc(N3Cc4c(cc(CN5CC6(CCN(CC7CCN(c8ccc9c(c8F)CN(C8CCC(=O)NC8=O)C9=O)CC7)CC6)C5)cc4C(F)(F)F)C3=O)c2)COC1] | 3-(4-fluoro-5-{4-[(2-{[2-(3-{3-[(4- methyl-4H-1,2,4-triazol-3- yl)methyl]oxetan-3-yl}phenyl)-3-oxo- 7-(trifluoromethyl)-2,3-dihydro-1H- isoindol-5-yl]methyl}-2,7- diazaspiro[3.5]nonan-7- yl)methyl]piperidin-1-yl}-1-oxo-2,3- dihydro-1H-isoindol-2-yl)piperidine- 2,6-dione | Example 4 |
| 26 | 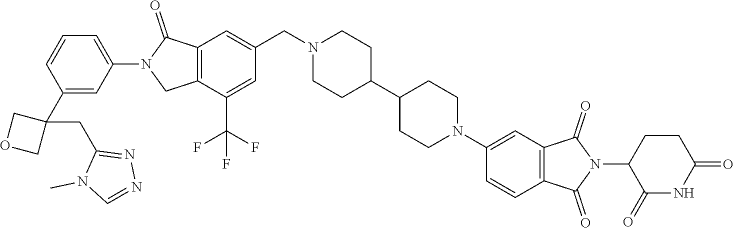 [SMILES: Cn1cnnc1CC1(c2cccc(N3Cc4c(cc(CN5CCC(C6CCN(c7ccc8c(c7)C(=O)N(C7CCC(=O)NC7=O)C8=O)CC6)CC5)cc4C(F)(F)F)C3=O)c2)COC1] | 2-(2,6-dioxopiperidin-3-yl)-5-(1′-{[2- (3-{3-[(4-methyl-4H-1,2,4-triazol-3- yl)methyl]oxetan-3-yl}phenyl)-3-oxo- 7-(trifluoromethyl)-2,3-dihydro-1H- isoindol-5-yl]methyl}-[4,4′- bipiperidin]-1-yl)-2,3-dihydro-1H- isoindole-1,3-dione | Scheme C |
| 27 | 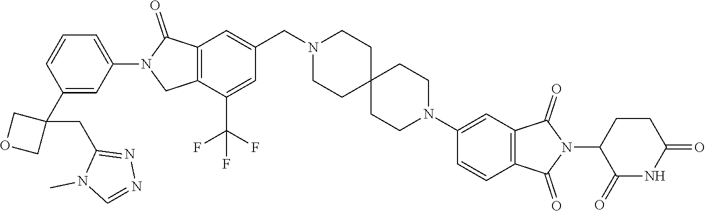 [SMILES: Cn1cnnc1CC1(c2cccc(N3Cc4c(cc(CN5CCC6(CC5)CCN(c5ccc7c(c5)C(=O)N(C5CCC(=O)NC5=O)C7=O)CC6)cc4C(F)(F)F)C3=O)c2)COC1] | 2-(2,6-dioxopiperidin-3-yl)-5-(9-{[2- (3-{3-[(4-methyl-4H-1,2,4-triazol-3- yl)methyl]oxetan-3-yl}phenyl)-3-oxo- 7-(trifluoromethyl)-2,3-dihydro-1H- isoindol-5-yl]methyl}-3,9- diazaspiro[5.5]undecan-3-yl)-2,3- dihydro-1H-isoindole-1,3-dione | Scheme C |
| 28 | 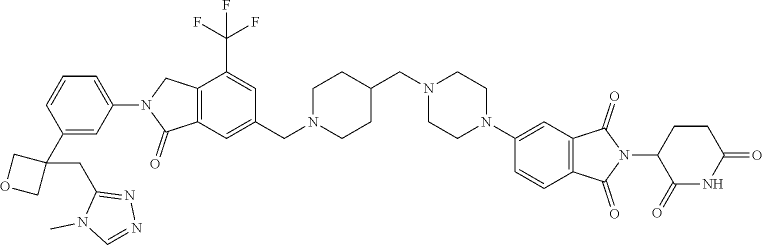 [SMILES: Cn1cnnc1CC1(c2cccc(N3Cc4c(cc(CN5CCC(CN6CCN(c7ccc8c(c7)C(=O)N(C7CCC(=O)NC7=O)C8=O)CC6)CC5)cc4C(F)(F)F)C3=O)c2)COC1] | 2-(2,6-dioxopiperidin-3-yl)-5-{4-[(1- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}piperidin-4- yl)methyl]piperazin-1-yl}-2,3-dihydro- 1H-isoindole-1,3-dione | Scheme C |
| 29 | 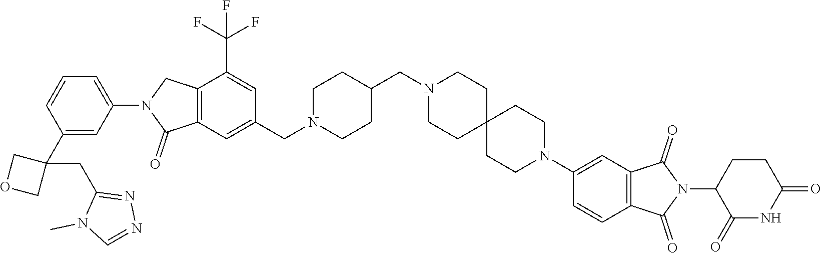 [SMILES: Cn1cnnc1CC1(c2cccc(N3Cc4c(cc(CN5CCC(CN6CCC7(CC6)CCN(c6ccc8c(c6)C(=O)N(C6CCC(=O)NC6=O)C8=O)CC7)CC5)cc4C(F)(F)F)C3=O)c2)COC1] | 2-(2,6-dioxopiperidin-3-yl)-5-{9-[(1- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}piperidin-4- yl)methyl]-3,9-diazaspiro[5.5]undecan- 3-yl}-2,3-dihydro-1H-isoindole-1,3- dione | Scheme C |
| 30 | 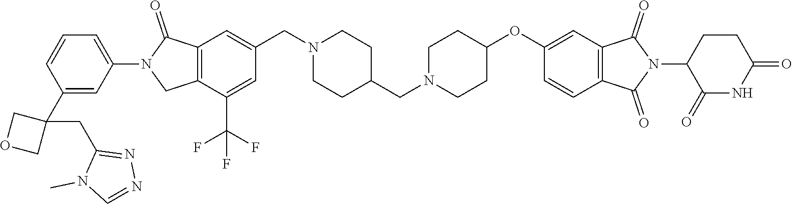 [SMILES: Cn1cnnc1CC1(c2cccc(N3Cc4c(cc(CN5CCC(CN6CCC(Oc7ccc8c(c7)C(=O)N(C7CCC(=O)NC7=O)C8=O)CC6)CC5)cc4C(F)(F)F)C3=O)c2)COC1] | 2-(2,6-dioxopiperidin-3-yl)-5-({1-[(1- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}piperidin-4- yl)methyl]piperidin-4-yl}oxy)-2,3- dihydro-1H-isoindole-1,3-dione | Scheme C |
| 31 | 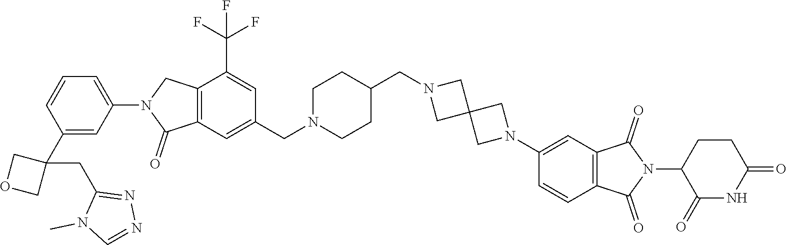 [SMILES: Cn1cnnc1CC1(c2cccc(N3Cc4c(cc(CN5CCC(CN6CC7(C6)CN(c6ccc8c(c6)C(=O)N(C6CCC(=O)NC6=O)C8=O)C7)CC5)cc4C(F)(F)F)C3=O)c2)COC1] | 2-(2,6-dioxopiperidin-3-yl)-5-{6-[(1- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}piperidin-4- yl)methyl]-2,6-diazaspiro[3.3]heptan- 2-yl}-2,3-dihydro-1H-isoindole-1,3- dione | Scheme C |
| 32 | 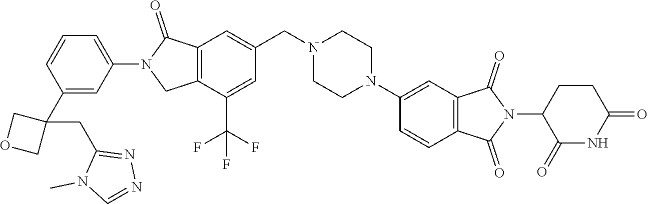 [SMILES: Cn1cnnc1CC1(c2cccc(N3Cc4c(cc(CN5CCN(c6ccc7c(c6)C(=O)N(C6CCC(=O)NC6=O)C7=O)CC5)cc4C(F)(F)F)C3=O)c2)COC1] | 2-(2,6-dioxopiperidin-3-yl)-5-(4-{[2- (3-{3-[(4-methyl-4H-1,2,4-triazol-3- yl)methyl]oxetan-3-yl}phenyl)-3-oxo- 7-(trifluoromethyl)-2,3-dihydro-1H- isoindol-5-yl]methyl}piperazin-1-yl)- 2,3-dihydro-1H-isoindole-1,3-dione | Scheme C |
| 33 | 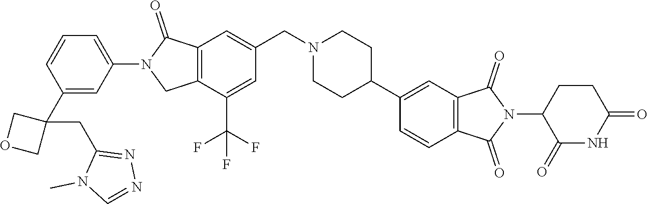 [SMILES: Cn1cnnc1CC1(c2cccc(N3Cc4c(cc(CN5CCC(c6ccc7c(c6)C(=O)N(C6CCC(=O)NC6=O)C7=O)CC5)cc4C(F)(F)F)C3=O)c2)COC1] | 2-(2,6-dioxopiperidin-3-yl)-5-(1-{[2- (3-{3-[(4-methyl-4H-1,2,4-triazol-3- yl)methyl]oxetan-3-yl}phenyl)-3-oxo- 7-(trifluoromethyl)-2,3-dihydro-1H- isoindol-5-yl]methyl}piperidin-4-yl)- 2,3-dihydro-1H-isoindole-1,3-dione | Scheme C |
| 34 | 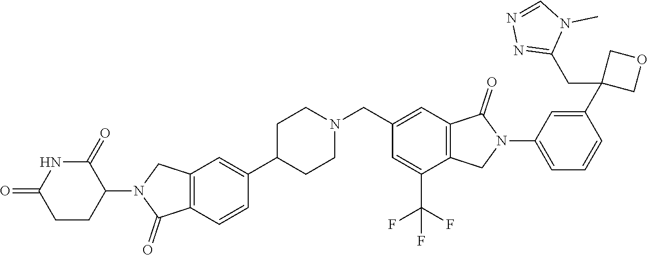 [SMILES: Cn1cnnc1CC1(c2cccc(N3Cc4c(cc(CN5CCC(c6ccc7c(c6)CN(C6CCC(=O)NC6=O)C7=O)CC5)cc4C(F)(F)F)C3=O)c2)COC1] | 3-[5-(1-{[2-(3-{3-[(4-methyl-4H-1,2,4- triazol-3-yl)methyl]oxetan-3- yl}phenyl)-3-oxo-7-(trifluoromethyl)- 2,3-dihydro-1H-isoindol-5- yl]methyl}piperidin-4-yl)-1-oxo-2,3- dihydro-1H-isoindol-2-yl]piperidine- 2,6-dione | Scheme C |
| 35 | 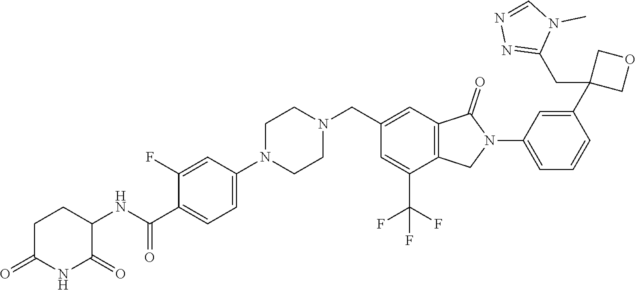 [SMILES: Cn1cnnc1CC1(c2cccc(N3Cc4c(cc(CN5CCN(c6ccc(C(=O)NC7CCC(=O)NC7=O)c(F)c6)CC5)cc4C(F)(F)F)C3=O)c2)COC1] | N-(2,6-dioxopiperidin-3-yl)-2-fluoro-4- (4-{[2-(3-{3-[(4-methyl-4H-1,2,4- triazol-3-yl)methyl]oxetan-3- yl}phenyl)-3-oxo-7-(trifluoromethyl)- 2,3-dihydro-1H-isoindol-5- yl]methyl}piperazin-1-yl)benzamide | Scheme C |
| 36 | 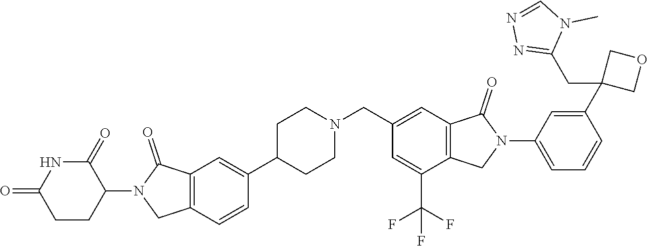 [SMILES: Cn1cnnc1CC1(c2cccc(N3Cc4c(cc(CN5CCC(c6ccc7c(c6)C(=O)N(C6CCC(=O)NC6=O)C7)CC5)cc4C(F)(F)F)C3=O)c2)COC1] | 3-[6-(1-{[2-(3-{3-[(4-methyl-4H-1,2,4- triazol-3-yl)methyl]oxetan-3- yl}phenyl)-3-oxo-7-(trifluoromethyl)- 2,3-dihydro-1H-isoindol-5- yl]methyl}piperidin-4-yl)-1-oxo-2,3- dihydro-1H-isoindol-2-yl]piperidine- 2,6-dione | Scheme C |
| 37 | 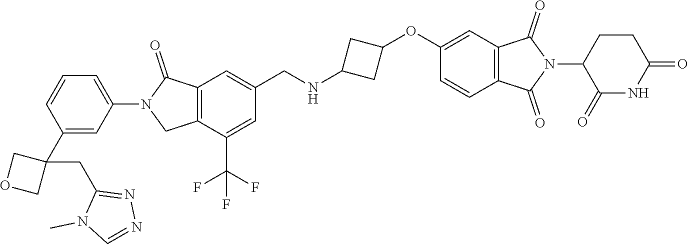 [SMILES: Cn1cnnc1CC1(c2cccc(N3Cc4c(cc(CNC5CC(Oc6ccc7c(c6)C(=O)N(C6CCC(=O)NC6=O)C7=O)C5)cc4C(F)(F)F)C3=O)c2)COC1] | 2-(2,6-dioxopiperidin-3-yl)-5-[3-({[2- (3-{3-[(4-methyl-4H-1,2,4-triazol-3- yl)methyl]oxetan-3-yl}phenyl)-3-oxo- 7-(trifluoromethyl)-2,3-dihydro-1H- isoindol-5- yl]methyl}amino)cyclobutoxy]-2,3- dihydro-1H-isoindole-1,3-dione | Scheme C |
| 38 | 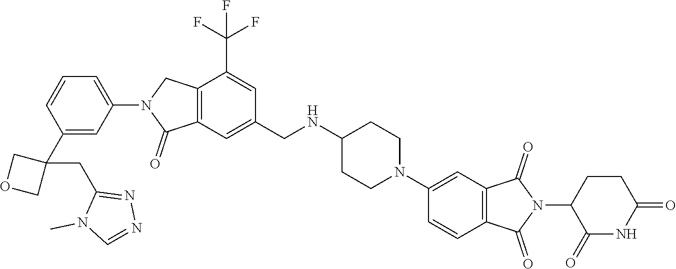 [SMILES: Cn1cnnc1CC1(c2cccc(N3Cc4c(cc(CNC5CCN(c6ccc7c(c6)C(=O)N(C6CCC(=O)NC6=O)C7=O)CC5)cc4C(F)(F)F)C3=O)c2)COC1] | 2-(2,6-dioxopiperidin-3-yl)-5-[4-({[2- (3-{3-[(4-methyl-4H-1,2,4-triazol-3- yl)methyl]oxetan-3-yl}phenyl)-3-oxo- 7-(trifluoromethyl)-2,3-dihydro-1H- isoindol-5-yl]methyl}amino)piperidin- 1-yl]-2,3-dihydro-1H-isoindole-1,3- dione | Scheme C |
| 39 | 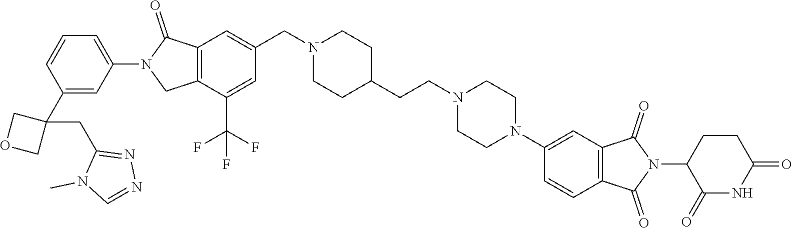 [SMILES: Cn1cnnc1CC1(c2cccc(N3Cc4c(cc(CN5CCC(CCN6CCN(c7ccc8c(c7)C(=O)N(C7CCC(=O)NC7=O)C8=O)CC6)CC5)cc4C(F)(F)F)C3=O)c2)COC1] | 2-(2,6-dioxopiperidin-3-yl)-5-{4-[2-(1- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}piperidin-4- yl)ethyl]piperazin-1-yl}-2,3-dihydro- 1H-isoindole-1,3-dione | Scheme C |
| 40 | 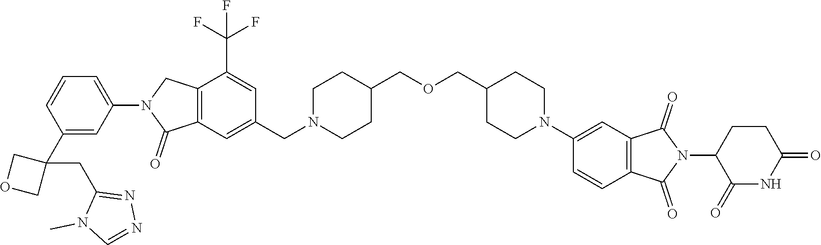 [SMILES: Cn1cnnc1CC1(c2cccc(N3Cc4c(cc(CN5CCC(COCC6CCN(c7ccc8c(c7)C(=O)N(C7CCC(=O)NC7=O)C8=O)CC6)CC5)cc4C(F)(F)F)C3=O)c2)COC1] | 2-(2,6-dioxopiperidin-3-yl)-5-(4-{[(1- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}piperidin-4- yl)methoxy]methyl}piperidin-1-yl)- 2,3-dihydro-1H-isoindole-1,3-dione | Scheme C |
| 41 | 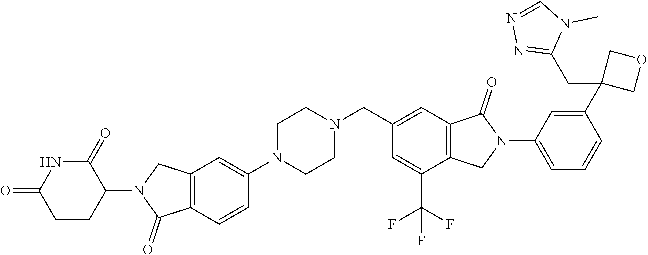 [SMILES: Cn1cnnc1CC1(c2cccc(N3Cc4c(cc(CN5CCN(c6ccc7c(c6)CN(C6CCC(=O)NC6=O)C7=O)CC5)cc4C(F)(F)F)C3=O)c2)COC1] | 3-[5-(4-{[2-(3-{3-[(4-methyl-4H-1,2,4- triazol-3-yl)methyl]oxetan-3- yl}phenyl)-3-oxo-7-(trifluoromethyl)- 2,3-dihydro-1H-isoindol-5- yl]methyl}piperazin-1-yl)-1-oxo-2,3- dihydro-1H-isoindol-2-yl]piperidine- 2,6-dione | Scheme C |
| 42 | 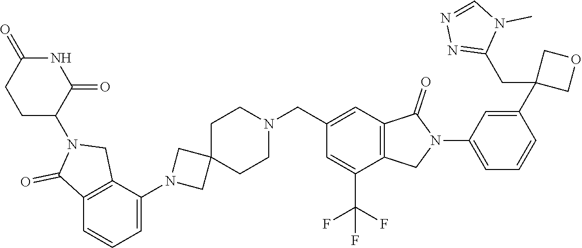 [SMILES: Cn1cnnc1CC1(c2cccc(N3Cc4c(cc(CN5CCC6(CC5)CN(c5cccc7c5CN(C5CCC(=O)NC5=O)C7=O)C6)cc4C(F)(F)F)C3=O)c2)COC1] | 3-[4-(7-{[2-(3-{3-[(4-methyl-4H-1,2,4- triazol-3-yl)methyl]oxetan-3- yl}phenyl)-3-oxo-7-(trifluoromethyl)- 2,3-dihydro-1H-isoindol-5-yl]methyl}- 2,7-diazaspiro[3.5]nonan-2-yl)-1-oxo- 2,3-dihydro-1H-isoindol-2- yl]piperidine-2,6-dione | Scheme C |
| 43 | 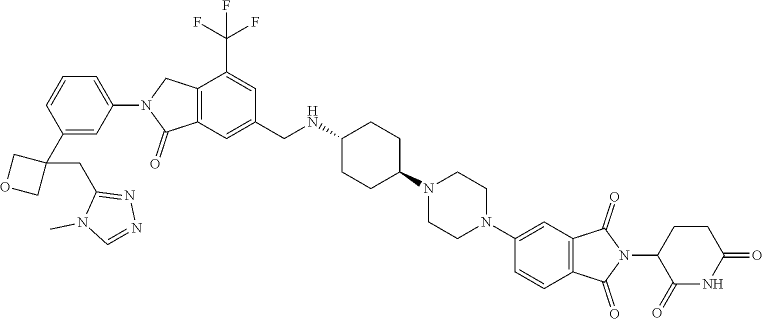 [SMILES: Cn1cnnc1CC1(c2cccc(N3Cc4c(cc(CN[C@H]5CC[C@H](N6CCN(c7ccc8c(c7)C(=O)N(C7CCC(=O)NC7=O)C8=O)CC6)CC5)cc4C(F)(F)F)C3=O)c2)COC1] | 2-(2,6-dioxopiperidin-3-yl)-5-{4- [(1r,4r)-4-({[2-(3-{3-[(4-methyl-4H- 1,2,4-triazol-3-yl)methyl]oxetan-3- yl}phenyl)-3-oxo-7-(trifluoromethyl)- 2,3-dihydro-1H-isoindol-5- yl]methyl}amino)cyclohexyl]piperazin- 1-yl}-2,3-dihydro-1H-isoindole-1,3- dione | Scheme C |
| 44 | 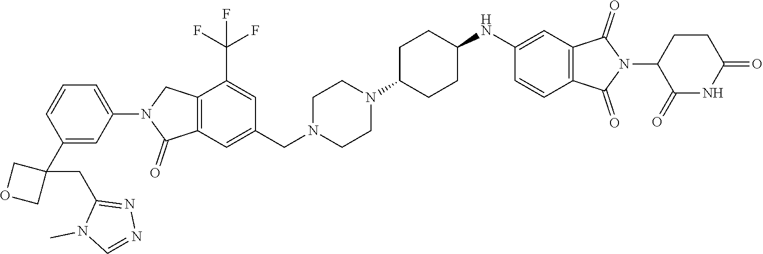 [SMILES: Cn1cnnc1CC1(c2cccc(N3Cc4c(cc(CN5CCN([C@H]6CC[C@H](Nc7ccc8c(c7)C(=O)N(C7CCC(=O)NC7=O)C8=O)CC6)CC5)cc4C(F)(F)F)C3=O)c2)COC1] | 2-(2,6-dioxopiperidin-3-yl)-5-{[(1r,4r)- 4-(4-{[2-(3-{3-[(4-methyl-4H-1,2,4- triazol-3-yl)methyl]oxetan-3- yl}phenyl)-3-oxo-7-(trifluoromethyl)- 2,3-dihydro-1H-isoindol-5- yl]methyl}piperazin-1- yl)cyclohexyl]amino}-2,3-dihydro-1H- isoindole-1,3-dione | Scheme C |
| 45 | 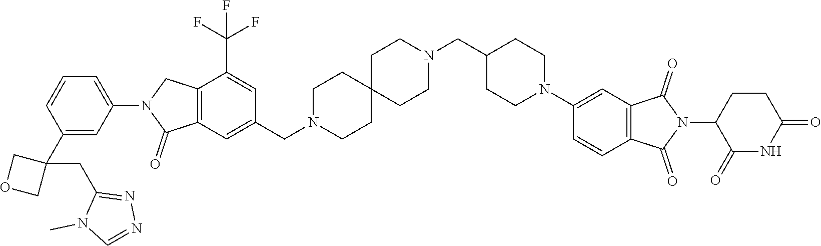 [SMILES: Cn1cnnc1CC1(c2cccc(N3Cc4c(cc(CN5CCC6(CC5)CCN(CC5CCN(c7ccc8c(c7)C(=O)N(C7CCC(=O)NC7=O)C8=O)CC5)CC6)cc4C(F)(F)F)C3=O)c2)COC1] | 2-(2,6-dioxopiperidin-3-yl)-5-{4-[(9- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}-3,9- diazaspiro[5.5]undecan-3- yl)methyl]piperidin-1-yl}-2,3-dihydro- 1H-isoindole-1,3-dione | Scheme C |
| 46 | 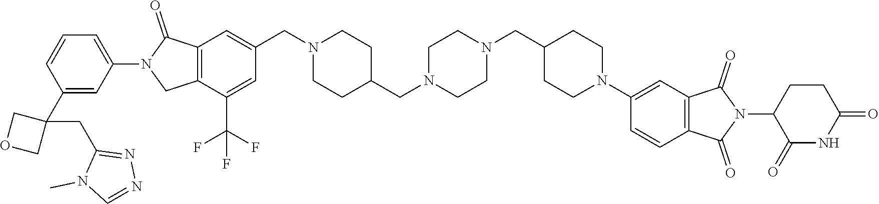 [SMILES: Cn1cnnc1CC1(c2cccc(N3Cc4c(cc(CN5CCC(CN6CCN(CC7CCN(c8ccc9c(c8)C(=O)N(C8CCC(=O)NC8=O)C9=O)CC7)CC6)CC5)cc4C(F)(F)F)C3=O)c2)COC1] | 2-(2,6-dioxopiperidin-3-yl)-5-[4-({4- [(1-{[2-(3-{3-[(4-methyl-4H-1,2,4- triazol-3-yl)methyl]oxetan-3- yl}phenyl)-3-oxo-7-(trifluoromethyl)- 2,3-dihydro-1H-isoindol-5- yl]methyl}piperidin-4- yl)methyl]piperazin-1- yl}methyl)piperidin-1-yl]-2,3-dihydro- 1H-isoindole-1,3-dione | Scheme C |
| 47 | 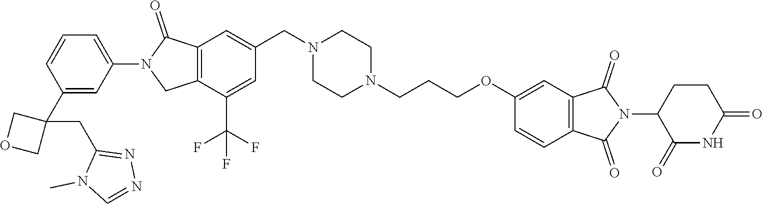 [SMILES: Cn1cnnc1CC1(c2cccc(N3Cc4c(cc(CN5CCN(CCCOc6ccc7c(c6)C(=O)N(C6CCC(=O)NC6=O)C7=O)CC5)cc4C(F)(F)F)C3=O)c2)COC1] | 2-(2,6-dioxopiperidin-3-yl)-5-[3-(4- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}piperazin-1- yl)propoxy]-2,3-dihydro-1H-isoindole- 1,3-dione | Scheme C |
| 48 | 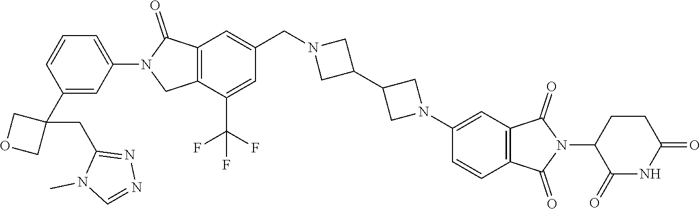 [SMILES: Cn1cnnc1CC1(c2cccc(N3Cc4c(cc(CN5CC(C6CN(c7ccc8c(c7)C(=O)N(C7CCC(=O)NC7=O)C8=O)C6)C5)cc4C(F)(F)F)C3=O)c2)COC1] | 2-(2,6-dioxopiperidin-3-yl)-5-(1′-{[2- (3-{3-[(4-methyl-4H-1,2,4-triazol-3- yl)methyl]oxetan-3-yl}phenyl)-3-oxo- 7-(trifluoromethyl)-2,3-dihydro-1H- isoindol-5-yl]methyl}-[3,3′-biazetidin]- 1-yl)-2,3-dihydro-1H-isoindole-1,3- dione | Scheme C |
| 49 | 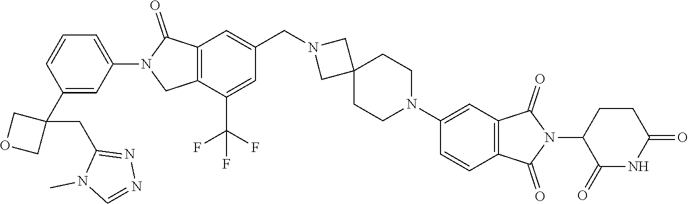 [SMILES: Cn1cnnc1CC1(c2cccc(N3Cc4c(cc(CN5CC6(CCN(c7ccc8c(c7)C(=O)N(C7CCC(=O)NC7=O)C8=O)CC6)C5)cc4C(F)(F)F)C3=O)c2)COC1] | 2-(2,6-dioxopiperidin-3-yl)-5-(2-{[2- (3-{3-[(4-methyl-4H-1,2,4-triazol-3- yl)methyl]oxetan-3-yl}phenyl)-3-oxo- 7-(trifluoromethyl)-2,3-dihydro-1H- isoindol-5-yl]methyl}-2,7- diazaspiro[3.5]nonan-7-yl)-2,3- dihydro-1H-isoindole-1,3-dione | Scheme C |
| 50 | 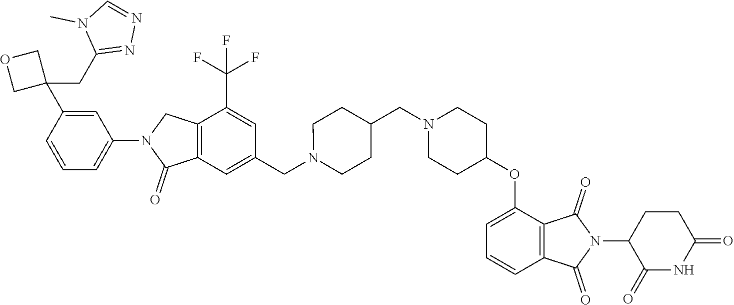 [SMILES: Cn1cnnc1CC1(c2cccc(N3Cc4c(cc(CN5CCC(CN6CCC(Oc7cccc8c7C(=O)N(C7CCC(=O)NC7=O)C8=O)CC6)CC5)cc4C(F)(F)F)C3=O)c2)COC1] | 2-(2,6-dioxopiperidin-3-yl)-4-({1-[(1- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}piperidin-4- yl)methyl]piperidin-4-yl}oxy)-2,3- dihydro-1H-isoindole-1,3-dione | Scheme C |
| 51 | 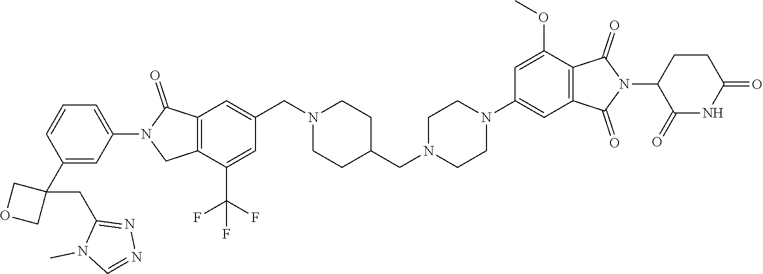 [SMILES: COc1cc(N2CCN(CC3CCN(Cc4cc5c(c(C(F)(F)F)c4)CN(c4cccc(C6(Cc7nncn7C)COC6)c4)C5=O)CC3)CC2)cc2c1C(=O)N(C1CCC(=O)NC1=O)C2=O] | 2-(2,6-dioxopiperidin-3-yl)-4-methoxy- 6-{4-[(1-{[2-(3-{3-[(4-methyl-4H- 1,2,4-triazol-3-yl)methyl]oxetan-3- yl}phenyl)-3-oxo-7-(trifluoromethyl)- 2,3-dihydro-1H-isoindol-5- yl]methyl}piperidin-4- yl)methyl]piperazin-1-yl}-2,3-dihydro- 1H-isoindole-1,3-dione | Scheme C |
| 52 | 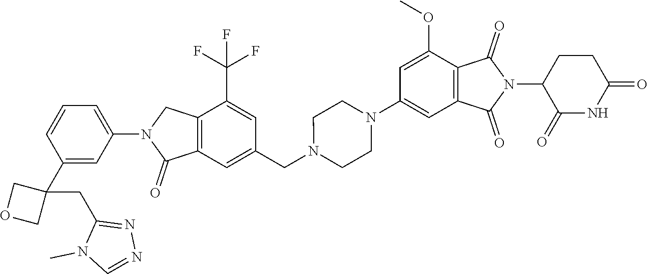 [SMILES: COc1cc(N2CCN(Cc3cc4c(c(C(F)(F)F)c3)CN(c3cccc(C5(Cc6nncn6C)COC5)c3)C4=O)CC2)cc2c1C(=O)N(C1CCC(=O)NC1=O)C2=O] | 2-(2,6-dioxopiperidin-3-yl)-4-methoxy- 6-(4-{[2-(3-{3-[(4-methyl-4H-1,2,4- triazol-3-yl)methyl]oxetan-3- yl}phenyl)-3-oxo-7-(trifluoromethyl)- 2,3-dihydro-1H-isoindol-5- yl]methyl}piperazin-1-yl)-2,3-dihydro- 1H-isoindole-1,3-dione | Scheme C |
| 53 | 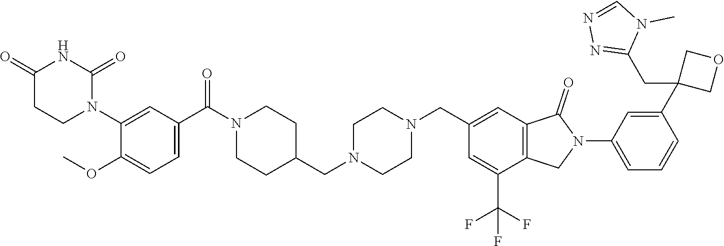 [SMILES: COc1ccc(C(=O)N2CCC(CN3CCN(Cc4cc5c(c(C(F)(F)F)c4)CN(c4cccc(C6(Cc7nncn7C)COC6)c4)C5=O)CC3)CC2)cc1N1CCC(=O)NC1=O] | 1-(2-methoxy-5-{4-[(4-{[2-(3-{3-[(4- methyl-4H-1,2,4-triazol-3- yl)methyl]oxetan-3-yl}phenyl)-3-oxo- 7-(trifluoromethyl)-2,3-dihydro-1H- isoindol-5-yl]methyl}piperazin-1- yl)methyl]piperidine-1- carbonyl}phenyl)-1,3-diazinane-2,4- dione | Scheme C |
| 54 | 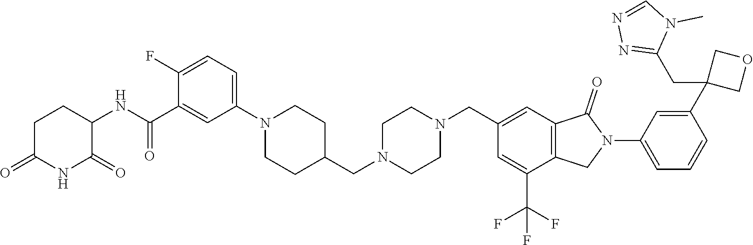 [SMILES: Cn1cnnc1CC1(c2cccc(N3Cc4c(cc(CN5CCN(CC6CCN(c7ccc(F)c(C(=O)NC8CCC(=O)NC8=O)c7)CC6)CC5)cc4C(F)(F)F)C3=O)c2)COC1] | N-(2,6-dioxopiperidin-3-yl)-2-fluoro-5- {4-[(4-{[2-(3-{3-[(4-methyl-4H-1,2,4- triazol-3-yl)methyl]oxetan-3- yl}phenyl)-3-oxo-7-(trifluoromethyl)- 2,3-dihydro-1H-isoindol-5- yl]methyl}piperazin-1- yl)methyl]piperidin-1-yl}benzamide | Scheme C |
| 55 | 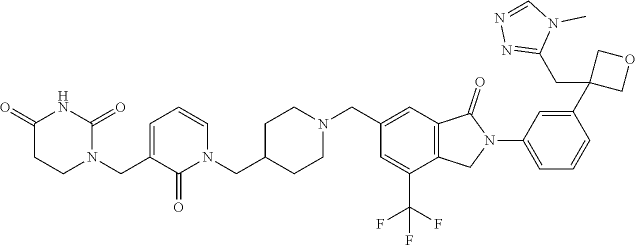 [SMILES: Cn1cnnc1CC1(c2cccc(N3Cc4c(cc(CN5CCC(Cn6cccc(CN7CCC(=O)NC7=O)c6=O)CC5)cc4C(F)(F)F)C3=O)c2)COC1] | 1-({1-[(1-{[2-(3-{3-[(4-methyl-4H- 1,2,4-triazol-3-yl)methyl]oxetan-3- yl}phenyl)-3-oxo-7-(trifluoromethyl)- 2,3-dihydro-1H-isoindol-5- yl]methyl}piperidin-4-yl)methyl]-2- oxo-1,2-dihydropyridin-3-yl}methyl)- 1,3-diazinane-2,4-dione | Scheme C |
| 56 | 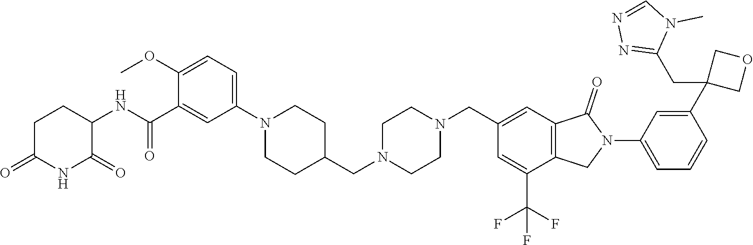 [SMILES: COc1ccc(N2CCC(CN3CCN(Cc4cc5c(c(C(F)(F)F)c4)CN(c4cccc(C6(Cc7nncn7C)COC6)c4)C5=O)CC3)CC2)cc1C(=O)NC1CCC(=O)NC1=O] | N-(2,6-dioxopiperidin-3-yl)-2- methoxy-5-{4-[(4-{[2-(3-{3-[(4- methyl-4H-1,2,4-triazol-3- yl)methyl]oxetan-3-yl}phenyl)-3-oxo- 7-(trifluoromethyl)-2,3-dihydro-1H- isoindol-5-yl]methyl}piperazin-1- yl)methyl]piperidin-1-yl}benzamide | Scheme C |
| 57 | 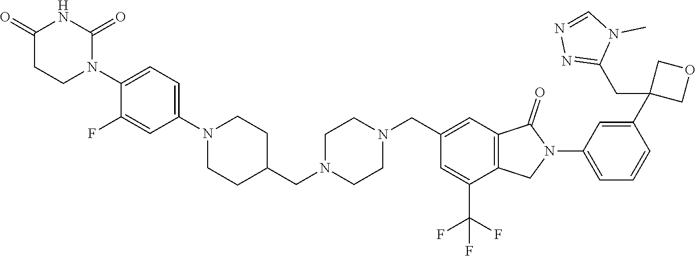 [SMILES: Cn1cnnc1CC1(c2cccc(N3Cc4c(cc(CN5CCN(CC6CCN(c7ccc(N8CCC(=O)NC8=O)c(F)c7)CC6)CC5)cc4C(F)(F)F)C3=O)c2)COC1] | 1-(2-fluoro-4-{4-[(4-{[2-(3-{3-[(4- methyl-4H-1,2,4-triazol-3- yl)methyl]oxetan-3-yl}phenyl)-3-oxo- 7-(trifluoromethyl)-2,3-dihydro-1H- isoindol-5-yl]methyl}piperazin-1- yl)methyl]piperidin-1-yl}phenyl)-1,3- diazinane-2,4-dione | Scheme C |
| 58 | 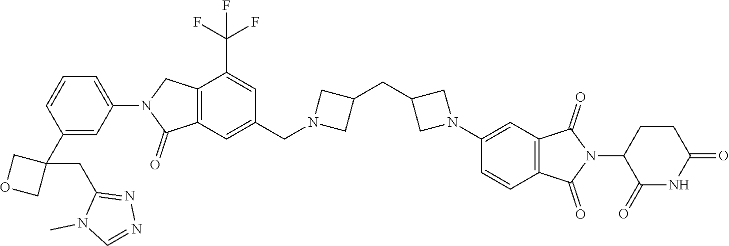 [SMILES: Cn1cnnc1CC1(c2cccc(N3Cc4c(cc(CN5CC(CC6CN(c7ccc8c(c7)C(=O)N(C7CCC(=O)NC7=O)C8=O)C6)C5)cc4C(F)(F)F)C3=O)c2)COC1] | 2-(2,6-dioxopiperidin-3-yl)-5-{3-[(1- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}azetidin-3- yl)methyl]azetidin-1-yl}-2,3-dihydro- 1H-isoindole-1,3-dione | Scheme C |
| 59 | 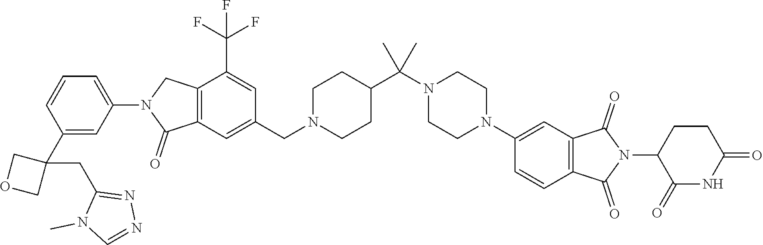 [SMILES: Cn1cnnc1CC1(c2cccc(N3Cc4c(cc(CN5CCC(C(C)(C)N6CCN(c7ccc8c(c7)C(=O)N(C7CCC(=O)NC7=O)C8=O)CC6)CC5)cc4C(F)(F)F)C3=O)c2)COC1] | 2-(2,6-dioxopiperidin-3-yl)-5-{4-[2-(1- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}piperidin-4- yl)propan-2-yl]piperazin-1-yl}-2,3- dihydro-1H-isoindole-1,3-dione | Scheme C |
| 60 | 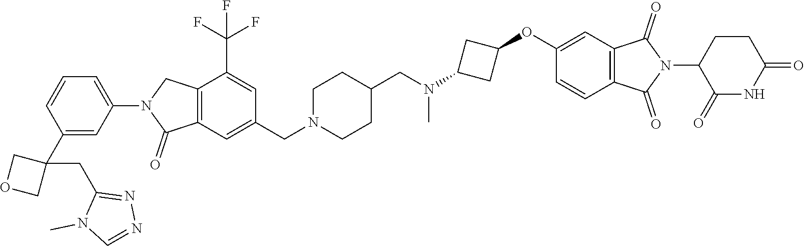 [SMILES: Cn1cnnc1CC1(c2cccc(N3Cc4c(cc(CN5CCC(CN(C)[C@H]6C[C@H](Oc7ccc8c(c7)C(=O)N(C7CCC(=O)NC7=O)C8=O)C6)CC5)cc4C(F)(F)F)C3=O)c2)COC1] | 2-(2,6-dioxopiperidin-3-yl)-5-[(1r,3r)- 3-{methyl[(1-{[2-(3-{3-[(4-methyl-4H- 1,2,4-triazol-3-yl)methyl]oxetan-3- yl}phenyl)-3-oxo-7-(trifluoromethyl)- 2,3-dihydro-1H-isoindol-5- yl]methyl}piperidin-4- yl)methyl]amino}cyclobutoxy]-2,3- dihydro-1H-isoindole-1,3-dione | Scheme C |
| 61 | 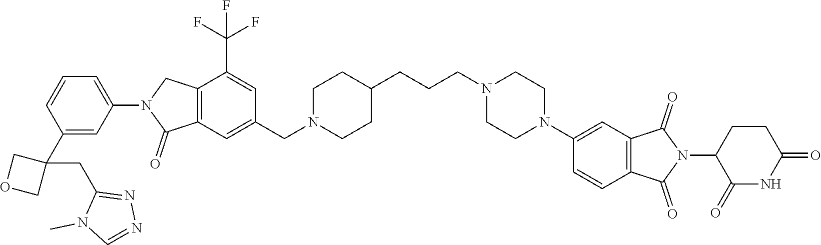 [SMILES: Cn1cnnc1CC1(c2cccc(N3Cc4c(cc(CN5CCC(CCCN6CCN(c7ccc8c(c7)C(=O)N(C7CCC(=O)NC7=O)C8=O)CC6)CC5)cc4C(F)(F)F)C3=O)c2)COC1] | 2-(2,6-dioxopiperidin-3-yl)-5-{4-[3-(1- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}piperidin-4- yl)propyl]piperazin-1-yl}-2,3-dihydro- 1H-isoindole-1,3-dione | Scheme C |
| 62 | 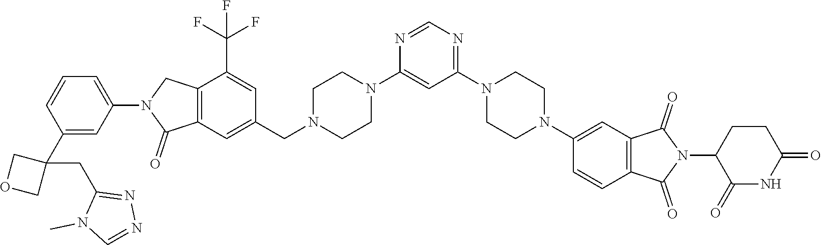 [SMILES: Cn1cnnc1CC1(c2cccc(N3Cc4c(cc(CN5CCN(c6cc(N7CCN(c8ccc9c(c8)C(=O)N(C8CCC(=O)NC8=O)C9=O)CC7)ncn6)CC5)cc4C(F)(F)F)C3=O)c2)COC1] | 2-(2,6-dioxopiperidin-3-yl)-5-{4-[6-(4- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}piperazin-1- yl)pyrimidin-4-yl]piperazin-1-yl}-2,3- dihydro-1H-isoindole-1,3-dione | Scheme C |
| 63 | 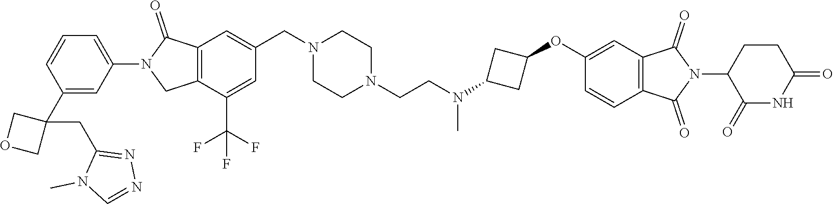 [SMILES: Cn1cnnc1CC1(c2cccc(N3Cc4c(cc(CN5CCN(CCN(C)[C@H]6C[C@H](Oc7ccc8c(c7)C(=O)N(C7CCC(=O)NC7=O)C8=O)C6)CC5)cc4C(F)(F)F)C3=O)c2)COC1] | 2-(2,6-dioxopiperidin-3-yl)-5-[(1r,3r)- 3-{methyl[2-(4-{[2-(3-{3-[(4-methyl- 4H-1,2,4-triazol-3-yl)methyl]oxetan-3- yl}phenyl)-3-oxo-7-(trifluoromethyl)- 2,3-dihydro-1H-isoindol-5- yl]methyl}piperazin-1- yl)ethyl]amino}cyclobutoxy]-2,3- dihydro-1H-isoindole-1,3-dione | Scheme C |
| 64 | 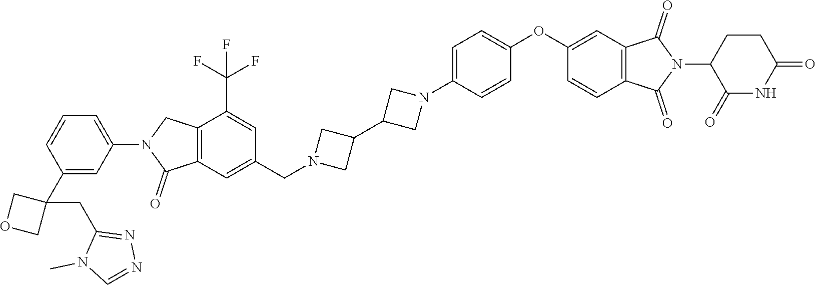 [SMILES: Cn1cnnc1CC1(c2cccc(N3Cc4c(cc(CN5CC(C6CN(c7ccc(Oc8ccc9c(c8)C(=O)N(C8CCC(=O)NC8=O)C9=O)cc7)C6)C5)cc4C(F)(F)F)C3=O)c2)COC1] | 2-(2,6-dioxopiperidin-3-yl)-5-[4-(1′- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}-[3,3′- biazetidin]-1-yl)phenoxy]-2,3-dihydro- 1H-isoindole-1,3-dione | Scheme C |
| 65 | 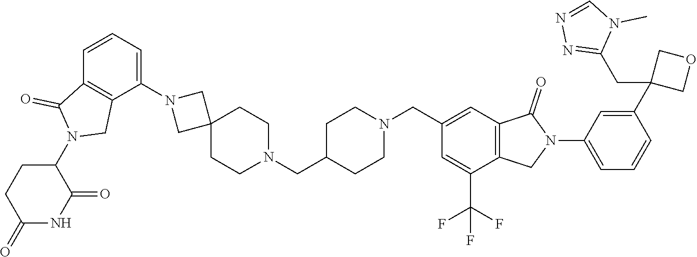 [SMILES: Cn1cnnc1CC1(c2cccc(N3Cc4c(cc(CN5CCC(CN6CCC7(CC6)CN(c6cccc8c6CN(C6CCC(=O)NC6=O)C8=O)C7)CC5)cc4C(F)(F)F)C3=O)c2)COC1] | 3-(4-{7-[(1-{[2-(3-{3-[(4-methyl-4H- 1,2,4-triazol-3-yl)methyl]oxetan-3- yl}phenyl)-3-oxo-7-(trifluoromethyl)- 2,3-dihydro-1H-isoindol-5- yl]methyl}piperidin-4-yl)methyl]-2,7- diazaspiro[3.5]nonan-2-yl}-1-oxo-2,3- dihydro-1H-isoindol-2-yl)piperidine- 2,6-dione | Scheme C |
| 66 | 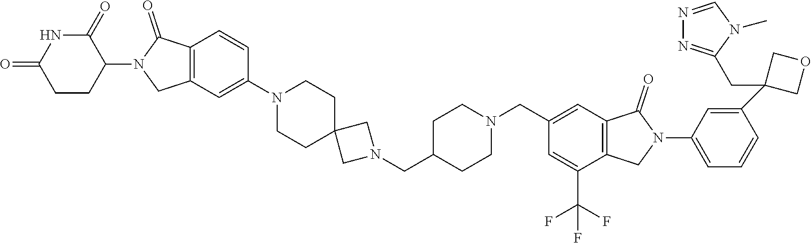 [SMILES: Cn1cnnc1CC1(c2cccc(N3Cc4c(cc(CN5CCC(CN6CC7(CCN(c8ccc9c(c8)CN(C8CCC(=O)NC8=O)C9=O)CC7)C6)CC5)cc4C(F)(F)F)C3=O)c2)COC1] | 3-(5-{2-[(1-{[2-(3-{3-[(4-methyl-4H- 1,2,4-triazol-3-yl)methyl]oxetan-3- yl}phenyl)-3-oxo-7-(trifluoromethyl)- 2,3-dihydro-1H-isoindol-5- yl]methyl}piperidin-4-yl)methyl]-2,7- diazaspiro[3.5]nonan-7-yl}-1-oxo-2,3- dihydro-1H-isoindol-2-yl)piperidine- 2,6-dione | Scheme C |
| 67 | 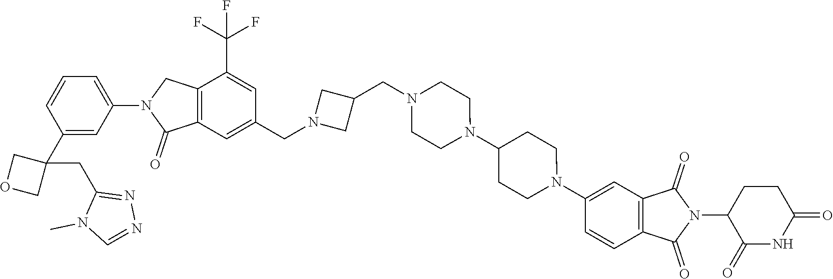 [SMILES: Cn1cnnc1CC1(c2cccc(N3Cc4c(cc(CN5CC(CN6CCN(C7CCN(c8ccc9c(c8)C(=O)N(C8CCC(=O)NC8=O)C9=O)CC7)CC6)C5)cc4C(F)(F)F)C3=O)c2)COC1] | 2-(2,6-dioxopiperidin-3-yl)-5-(4-{4- [(1-{[2-(3-{3-[(4-methyl-4H-1,2,4- triazol-3-yl)methyl]oxetan-3- yl}phenyl)-3-oxo-7-(trifluoromethyl)- 2,3-dihydro-1H-isoindol-5- yl]methyl}azetidin-3- yl)methyl]piperazin-1-yl}piperidin-1- yl)-2,3-dihydro-1H-isoindole-1,3-dione | Scheme C |
| 68 | 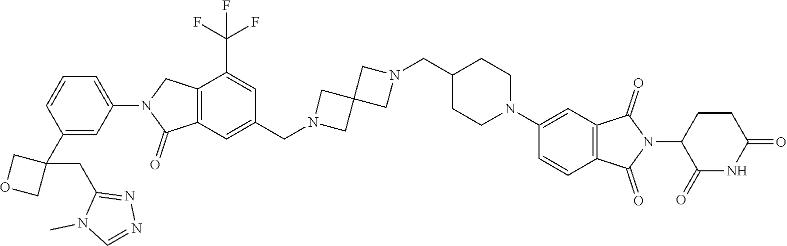 [SMILES: Cn1cnnc1CC1(c2cccc(N3Cc4c(cc(CN5CC6(C5)CN(CC5CCN(c7ccc8c(c7)C(=O)N(C7CCC(=O)NC7=O)C8=O)CC5)C6)cc4C(F)(F)F)C3=O)c2)COC1] | 2-(2,6-dioxopiperidin-3-yl)-5-{4-[(6- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}-2,6- diazaspiro[3.3]heptan-2- yl)methyl]piperidin-1-yl}-2,3-dihydro- 1H-isoindole-1,3-dione | Scheme C |
| 69 | 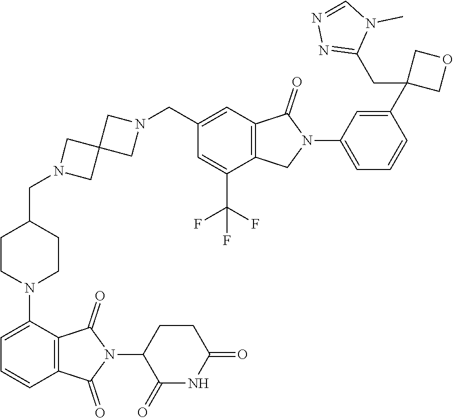 [SMILES: Cn1cnnc1CC1(c2cccc(N3Cc4c(cc(CN5CC6(C5)CN(CC5CCN(c7cccc8c7C(=O)N(C7CCC(=O)NC7=O)C8=O)CC5)C6)cc4C(F)(F)F)C3=O)c2)COC1] | 2-(2,6-dioxopiperidin-3-yl)-4-{4-[(6- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}-2,6- diazaspiro[3.3]heptan-2- yl)methyl]piperidin-1-yl}-2,3-dihydro- 1H-isoindole-1,3-dione | Scheme C |
| 70 | 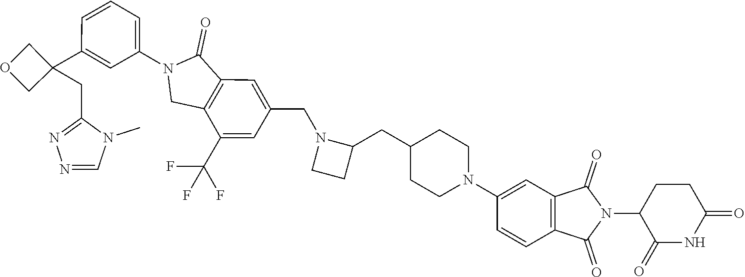 [SMILES: Cn1cnnc1CC1(c2cccc(N3Cc4c(cc(CN5CCC5CC5CCN(c6ccc7c(c6)C(=O)N(C6CCC(=O)NC6=O)C7=O)CC5)cc4C(F)(F)F)C3=O)c2)COC1] | 2-(2,6-dioxopiperidin-3-yl)-5-{4-[(1- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}azetidin-2- yl)methyl]piperidin-1-yl}-2,3-dihydro- 1H-isoindole-1,3-dione | Scheme C |
| 71 | 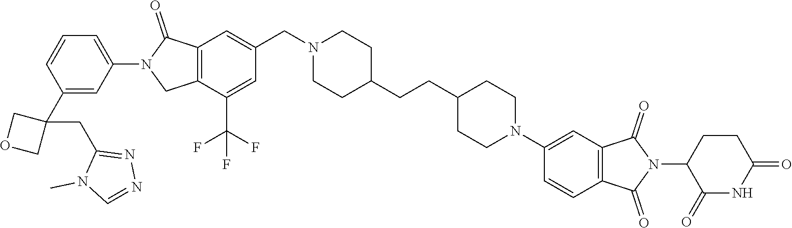 [SMILES: Cn1cnnc1CC1(c2cccc(N3Cc4c(cc(CN5CCC(CCC6CCN(c7ccc8c(c7)C(=O)N(C7CCC(=O)NC7=O)C8=O)CC6)CC5)cc4C(F)(F)F)C3=O)c2)COC1] | 2-(2,6-dioxopiperidin-3-yl)-5-{4-[2-(1- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}piperidin-4- yl)ethyl]piperidin-1-yl}-2,3-dihydro- 1H-isoindole-1,3-dione | Scheme C |
| 72 | 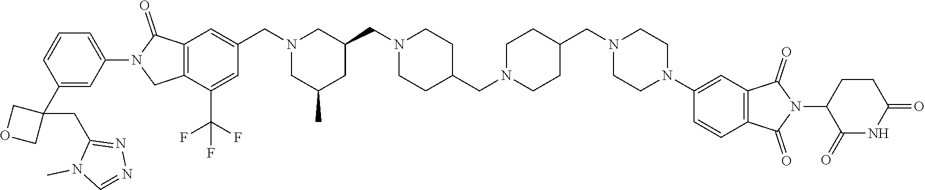 [SMILES: C[C@@H]1C[C@H](CN2CCC(CN3CCC(CN4CCN(c5ccc6c(c5)C(=O)N(C5CCC(=O)NC5=O)C6=O)CC4)CC3)CC2)CN(Cc2cc3c(c(C(F)(F)F)c2)CN(c2cccc(C4(Cc5nncn5C)COC4)c2)C3=O)C1] | 2-(2,6-dioxopiperidin-3-yl)-5-[4-({1- [(1-{[(3R,5R)-5-methyl-1-{[2-(3-{3- [(4-methyl-4H-1,2,4-triazol-3- yl)methyl]oxetan-3-yl}phenyl)-3-oxo- 7-(trifluoromethyl)-2,3-dihydro-1H- isoindol-5-yl]methyl}piperidin-3- yl]methyl}piperidin-4- yl)methyl]piperidin-4- yl}methyl)piperazin-1-yl]-2,3-dihydro- 1H-isoindole-1,3-dione | Example 74 |
| 73 | 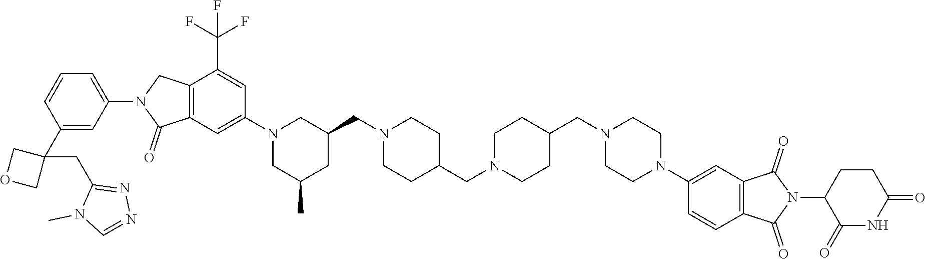 [SMILES: C[C@@H]1C[C@H](CN2CCC(CN3CCC(CN4CCN(c5ccc6c(c5)C(=O)N(C5CCC(=O)NC5=O)C6=O)CC4)CC3)CC2)CN(c2cc3c(c(C(F)(F)F)c2)CN(c2cccc(C4(Cc5nncn5C)COC4)c2)C3=O)C1] | 2-(2,6-dioxopiperidin-3-yl)-5-[4-({1- [(1-{[(3R,5R)-5-methyl-1-[2-(3-{3-[(4- methyl-4H-1,2,4-triazol-3- yl)methyl]oxetan-3-yl}phenyl)-3-oxo- 7-(trifluoromethyl)-2,3-dihydro-1H- isoindol-5-yl]piperidin-3- yl]methyl}piperidin-4- yl)methyl]piperidin-4- yl}methyl)piperazin-1-yl]-2,3-dihydro- 1H-isoindole-1,3-dione | Example 16 |
| 74 | 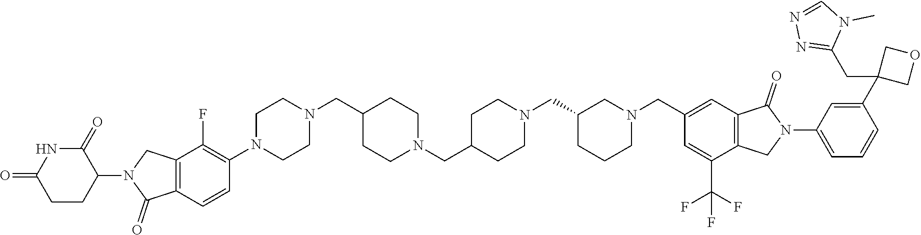 [SMILES: Cn1cnnc1CC1(c2cccc(N3Cc4c(cc(CN5CCC[C@H](CN6CCC(CN7CCC(CN8CCN(c9ccc%10c(c9F)CN(C9CCC(=O)NC9=O)C%10=O)CC8)CC7)CC6)C5)cc4C(F)(F)F)C3=O)c2)COC1] | 3-{4-fluoro-5-[4-({1-[(1-{[(3R)-1-{[2- (3-{3-[(4-methyl-4H-1,2,4-triazol-3- yl)methyl]oxetan-3-yl}phenyl)-3-oxo- 7-(trifluoromethyl)-2,3-dihydro-1H- isoindol-5-yl]methyl}piperidin-3- yl]methyl}piperidin-4- yl)methyl]piperidin-4- yl}methyl)piperazin-1-yl]-1-oxo-2,3- dihydro-1H-isoindol-2-yl}piperidine- 2,6-dione | Included |
| 75 | 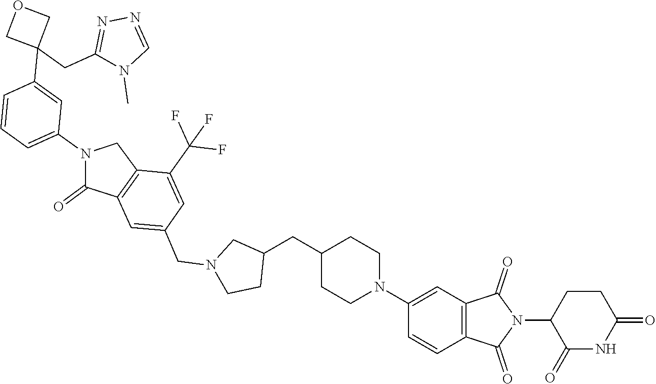 [SMILES: Cn1cnnc1CC1(c2cccc(N3Cc4c(cc(CN5CCC(CC6CCN(c7ccc8c(c7)C(=O)N(C7CCC(=O)NC7=O)C8=O)CC6)C5)cc4C(F)(F)F)C3=O)c2)COC1] | 2-(2,6-dioxopiperidin-3-yl)-5-{4-[(1- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}pyrrolidin-3- yl)methyl]piperidin-1-yl}-2,3-dihydro- 1H-isoindole-1,3-dione | Scheme A |
| 76 | 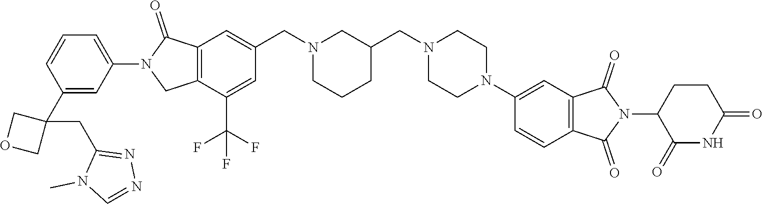 [SMILES: Cn1cnnc1CC1(c2cccc(N3Cc4c(cc(CN5CCCC(CN6CCN(c7ccc8c(c7)C(=O)N(C7CCC(=O)NC7=O)C8=O)CC6)C5)cc4C(F)(F)F)C3=O)c2)COC1] | 2-(2,6-dioxopiperidin-3-yl)-5-{4-[(1- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}piperidin-3- yl)methyl]piperazin-1-yl}-2,3-dihydro- 1H-isoindole-1,3-dione | Scheme A |
| 77 | 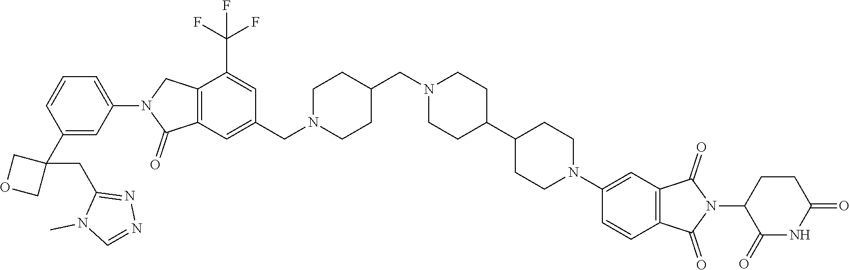 [SMILES: Cn1cnnc1CC1(c2cccc(N3Cc4c(cc(CN5CCC(CN6CCC(C7CCN(c8ccc9c(c8)C(=O)N(C8CCC(=O)NC8=O)C9=O)CC7)CC6)CC5)cc4C(F)(F)F)C3=O)c2)COC1] | 2-(2,6-dioxopiperidin-3-yl)-5-{1′-[(1- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}piperidin-4- yl)methyl]-[4,4′-bipiperidin]-1-yl}-2,3- dihydro-1H-isoindole-1,3-dione | Scheme A |
| 78 | 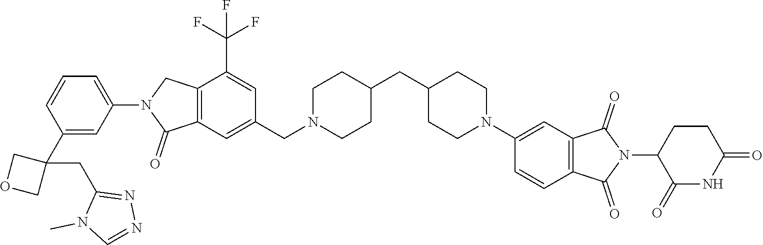 [SMILES: Cn1cnnc1CC1(c2cccc(N3Cc4c(cc(CN5CCC(CC6CCN(c7ccc8c(c7)C(=O)N(C7CCC(=O)NC7=O)C8=O)CC6)CC5)cc4C(F)(F)F)C3=O)c2)COC1] | 2-(2,6-dioxopiperidin-3-yl)-5-{4-[(1- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}piperidin-4- yl)methyl]piperidin-1-yl}-2,3-dihydro- 1H-isoindole-1,3-dione | Scheme A |
| 79 | 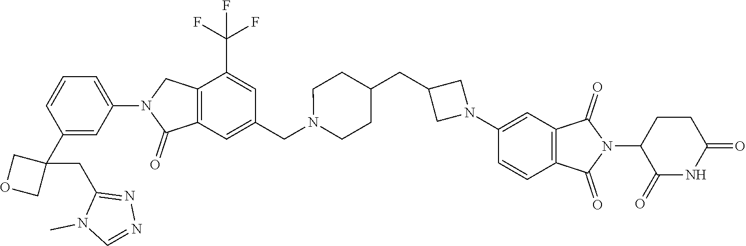 [SMILES: Cn1cnnc1CC1(c2cccc(N3Cc4c(cc(CN5CCC(CC6CN(c7ccc8c(c7)C(=O)N(C7CCC(=O)NC7=O)C8=O)C6)CC5)cc4C(F)(F)F)C3=O)c2)COC1] | 2-(2,6-dioxopiperidin-3-yl)-5-{3-[(1- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}piperidin-4- yl)methyl]azetidin-1-yl}-2,3-dihydro- 1H-isoindole-1,3-dione | Scheme A |
| 80 | 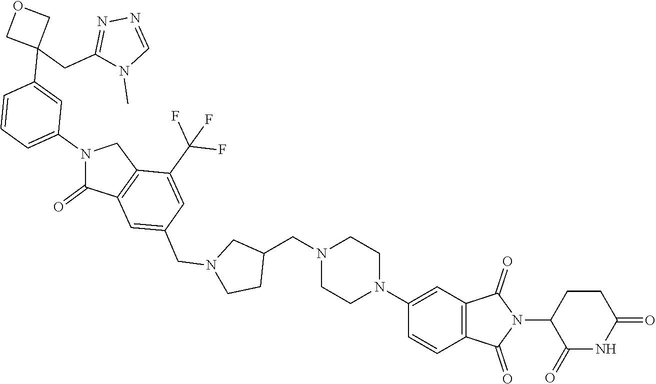 [SMILES: Cn1cnnc1CC1(c2cccc(N3Cc4c(cc(CN5CCC(CN6CCN(c7ccc8c(c7)C(=O)N(C7CCC(=O)NC7=O)C8=O)CC6)C5)cc4C(F)(F)F)C3=O)c2)COC1] | 2-(2,6-dioxopiperidin-3-yl)-5-{4-[(1- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}pyrrolidin-3- yl)methyl]piperazin-1-yl}-2,3-dihydro- 1H-isoindole-1,3-dione | Scheme A |
| 81 | 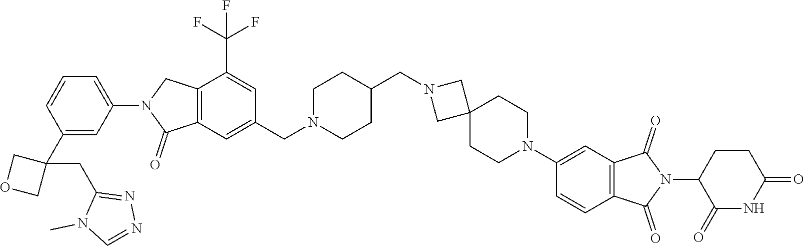 [SMILES: Cn1cnnc1CC1(c2cccc(N3Cc4c(cc(CN5CCC(CN6CC7(CCN(c8ccc9c(c8)C(=O)N(C8CCC(=O)NC8=O)C9=O)CC7)C6)CC5)cc4C(F)(F)F)C3=O)c2)COC1] | 2-(2,6-dioxopiperidin-3-yl)-5-{2-[(1- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}piperidin-4- yl)methyl]-2,7-diazaspiro[3.5]nonan-7- yl}-2,3-dihydro-1H-isoindole-1,3-dione | Scheme A |
| 82 | 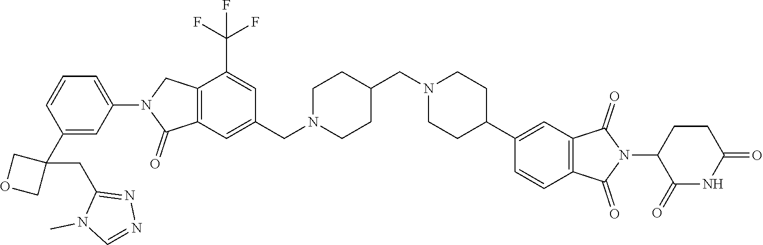 [SMILES: Cn1cnnc1CC1(c2cccc(N3Cc4c(cc(CN5CCC(CN6CCC(c7ccc8c(c7)C(=O)N(C7CCC(=O)NC7=O)C8=O)CC6)CC5)cc4C(F)(F)F)C3=O)c2)COC1] | 2-(2,6-dioxopiperidin-3-yl)-5-{1-[(1- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}piperidin-4- yl)methyl]piperidin-4-yl}-2,3-dihydro- 1H-isoindole-1,3-dione | Scheme A |
| 83 | 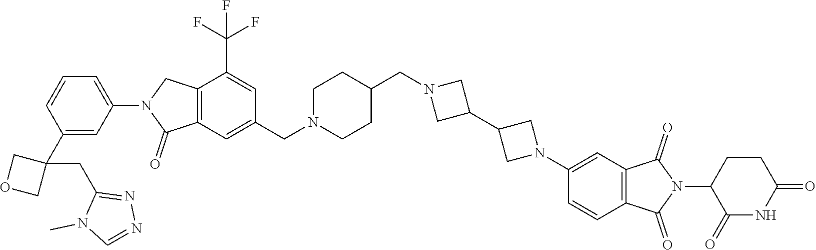 [SMILES: Cn1cnnc1CC1(c2cccc(N3Cc4c(cc(CN5CCC(CN6CC(C7CN(c8ccc9c(c8)C(=O)N(C8CCC(=O)NC8=O)C9=O)C7)C6)CC5)cc4C(F)(F)F)C3=O)c2)COC1] | 2-(2,6-dioxopiperidin-3-yl)-5-{1′-[(1- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}piperidin-4- yl)methyl]-[3,3′-biazetidin]-1-yl}-2,3- dihydro-1H-isoindole-1,3-dione | Scheme A |
| 84 | 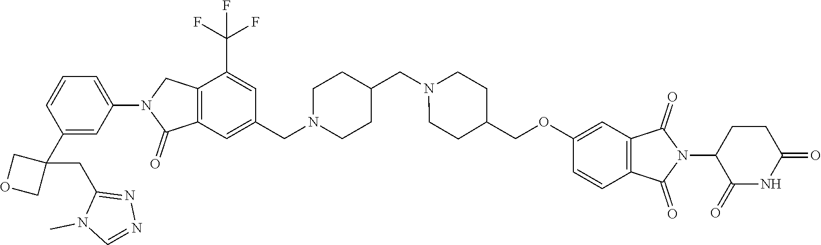 [SMILES: Cn1cnnc1CC1(c2cccc(N3Cc4c(cc(CN5CCC(CN6CCC(COc7ccc8c(c7)C(=O)N(C7CCC(=O)NC7=O)C8=O)CC6)CC5)cc4C(F)(F)F)C3=O)c2)COC1] | 2-(2,6-dioxopiperidin-3-yl)-5-({1-[(1- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}piperidin-4- yl)methyl]piperidin-4-yl}methoxy)- 2,3-dihydro-1H-isoindole-1,3-dione | Scheme A |
| 85 | 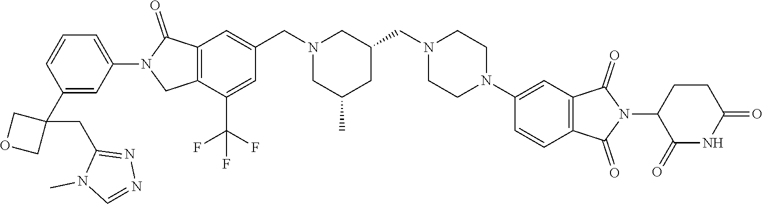 [SMILES: C[C@H]1C[C@@H](CN2CCN(c3ccc4c(c3)C(=O)N(C3CCC(=O)NC3=O)C4=O)CC2)CN(Cc2cc3c(c(C(F)(F)F)c2)CN(c2cccc(C4(Cc5nncn5C)COC4)c2)C3=O)C1] | 2-(2,6-dioxopiperidin-3-yl)-5-(4- {[(3R,5S)-5-methyl-1-{[2-(3-{3-[(4- methyl-4H-1,2,4-triazol-3- yl)methyl]oxetan-3-yl}phenyl)-3-oxo- 7-(trifluoromethyl)-2,3-dihydro-1H- isoindol-5-yl]methyl}piperidin-3- yl]methyl}piperazin-1-yl)-2,3-dihydro- 1H-isoindole-1,3-dione | Scheme A |
| 86 | 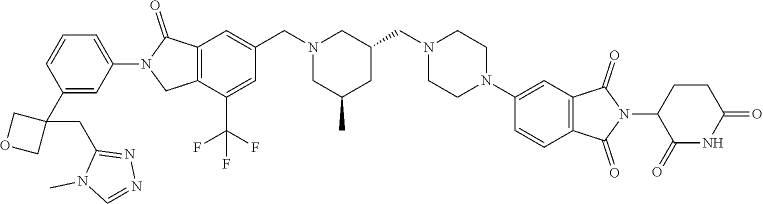 [SMILES: C[C@@H]1C[C@@H](CN2CCN(c3ccc4c(c3)C(=O)N(C3CCC(=O)NC3=O)C4=O)CC2)CN(Cc2cc3c(c(C(F)(F)F)c2)CN(c2cccc(C4(Cc5nncn5C)COC4)c2)C3=O)C1] | 2-(2,6-dioxopiperidin-3-yl)-5-(4- {[(3R,5R)-5-methyl-1-{[2-(3-{3-[(4- methyl-4H-1,2,4-triazol-3- yl)methyl]oxetan-3-yl}phenyl)-3-oxo- 7-(trifluoromethyl)-2,3-dihydro-1H- isoindol-5-yl]methyl}piperidin-3- yl]methyl}piperazin-1-yl)-2,3-dihydro- 1H-isoindole-1,3-dione | Scheme A |
| 87 | 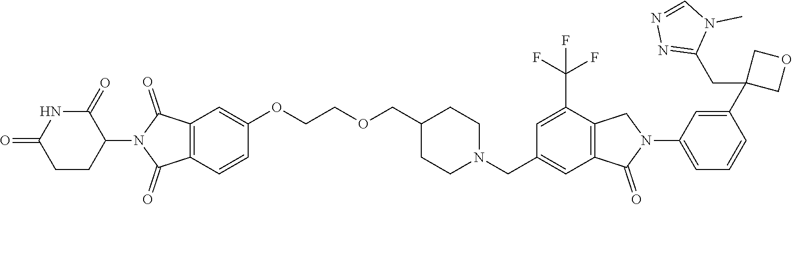 | 2-(2,6-dioxopiperidin-3-yl)-5-(2-((1- ((2-(3-(3-((4-methyl-4H-1,2,4-triazol- 3-yl)methyl)oxetan-3-yl)phenyl)-3- oxo-7-(trifluoromethyl)isoindolin-5- yl)methyl)piperidin-4- yl)methoxy)ethoxy)isoindoline-1,3- dione | Scheme A |
| 88 | 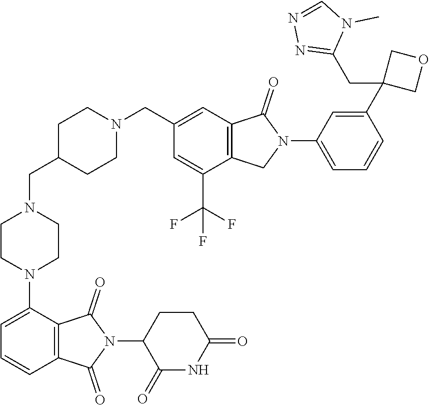 [SMILES: Cn1cnnc1CC1(c2cccc(N3Cc4c(cc(CN5CCC(CN6CCN(c7cccc8c7C(=O)N(C7CCC(=O)NC7=O)C8=O)CC6)CC5)cc4C(F)(F)F)C3=O)c2)COC1] | 2-(2,6-dioxopiperidin-3-yl)-4-(4-((1- ((2-(3-(3-((4-methyl-4H-1,2,4-triazol- 3-yl)methyl)oxetan-3-yl)phenyl)-3- oxo-7-(trifluoromethyl)isoindolin-5- yl)methyl)piperidin-4- yl)methyl)piperazin-1-yl)isoindoline- 1,3-dione | Scheme A |
| 89 | 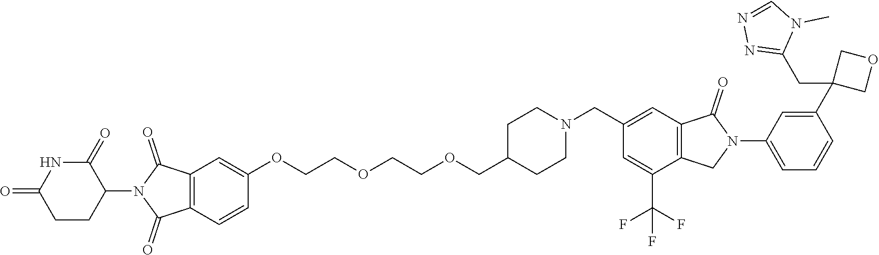 [SMILES: Cn1cnnc1CC1(c2cccc(N3Cc4c(cc(CN5CCC(COCCOCCOc6ccc7c(c6)C(=O)N(C6CCC(=O)NC6=O)C7=O)CC5)cc4C(F)(F)F)C3=O)c2)COC1] | 2-(2,6-dioxopiperidin-3-yl)-5-(2-{2- [(1-{[2-(3-{3-[(4-methyl-4H-1,2,4- triazol-3-yl)methyl]oxetan-3- yl}phenyl)-3-oxo-7-(trifluoromethyl)- 2,3-dihydro-1H-isoindol-5- yl]methyl}piperidin-4- yl)methoxy]ethoxy}ethoxy)-2,3- dihydro-1H-isoindole-1,3-dione | Scheme A |
| 90 | 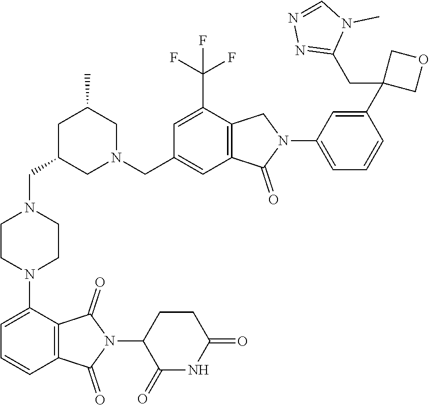 [SMILES: C[C@H]1C[C@@H](CN2CCN(c3cccc4c3C(=O)N(C3CCC(=O)NC3=O)C4=O)CC2)CN(Cc2cc3c(c(C(F)(F)F)c2)CN(c2cccc(C4(Cc5nncn5C)COC4)c2)C3=O)C1] | 2-(2,6-dioxopiperidin-3-yl)-4-(4- {[(3R,5S)-5-methyl-1-{[2-(3-{3-[(4- methyl-4H-1,2,4-triazol-3- yl)methyl]oxetan-3-yl}phenyl)-3-oxo- 7-(trifluoromethyl)-2,3-dihydro-1H- isoindol-5-yl]methyl}piperidin-3- yl]methyl}piperazin-1-yl)-2,3-dihydro- 1H-isoindole-1,3-dione | Scheme A |
| 91 | 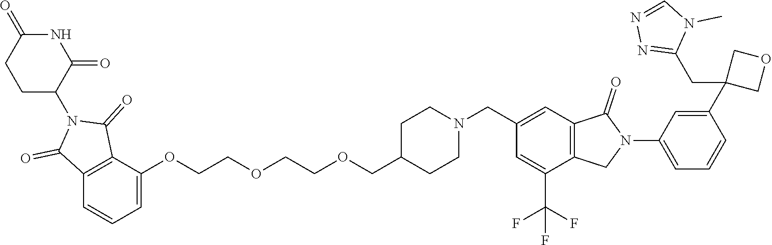 [SMILES: Cn1cnnc1CC1(c2cccc(N3Cc4c(cc(CN5CCC(COCCOCCOc6cccc7c6C(=O)N(C6CCC(=O)NC6=O)C7=O)CC5)cc4C(F)(F)F)C3=O)c2)COC1] | 2-(2,6-dioxopiperidin-3-yl)-4-(2-{2- [(1-{[2-(3-{3-[(4-methyl-4H-1,2,4- triazol-3-yl)methyl]oxetan-3- yl}phenyl)-3-oxo-7-(trifluoromethyl)- 2,3-dihydro-1H-isoindol-5- yl]methyl}piperidin-4- yl)methoxy]ethoxy}ethoxy)-2,3- dihydro-1H-isoindole-1,3-dione | Scheme A |
| 92 | 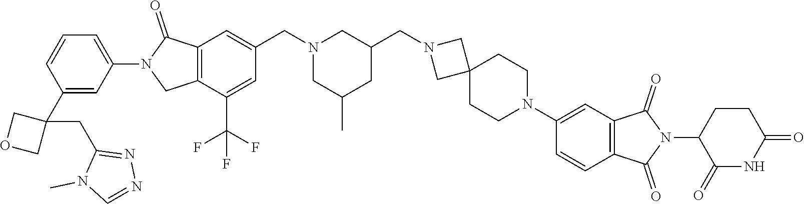 [SMILES: CC1CC(CN2CC3(CCN(c4ccc5c(c4)C(=O)N(C4CCC(=O)NC4=O)C5=O)CC3)C2)CN(Cc2cc3c(c(C(F)(F)F)c2)CN(c2cccc(C4(Cc5nncn5C)COC4)c2)C3=O)C1] | 2-(2,6-dioxopiperidin-3-yl)-5-{2-[(5- methyl-1-{[2-(3-{3-[(4-methyl-4H- 1,2,4-triazol-3-yl)methyl]oxetan-3- yl}phenyl)-3-oxo-7-(trifluoromethyl)- 2,3-dihydro-1H-isoindol-5- yl]methyl}piperidin-3-yl)methyl]-2,7- diazaspiro[3.5]nonan-7-yl}-2,3- dihydro-1H-isoindole-1,3-dione | Scheme A |
| 93 | 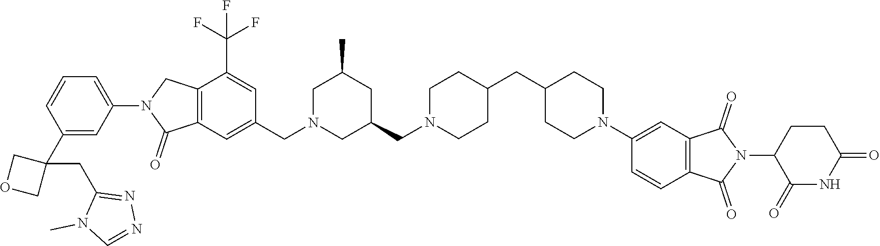 [SMILES: C[C@H]1C[C@@H](CN2CCC(CC3CCN(c4ccc5c(c4)C(=O)N(C4CCC(=O)NC4=O)C5=O)CC3)CC2)CN(Cc2cc3c(c(C(F)(F)F)c2)CN(c2cccc(C4(Cc5nncn5C)COC4)c2)C3=O)C1] | 2-(2,6-dioxopiperidin-3-yl)-5-{4-[(1- {[(3S,5S)-5-methyl-1-{[2-(3-{3-[(4- methyl-4H-1,2,4-triazol-3- yl)methyl]oxetan-3-yl}phenyl)-3-oxo- 7-(trifluoromethyl)-2,3-dihydro-1H- isoindol-5-yl]methyl}piperidin-3- yl]methyl}piperidin-4- yl)methyl]piperidin-1-yl}-2,3-dihydro- 1H-isoindole-1,3-dione | Scheme A |
| 94 | 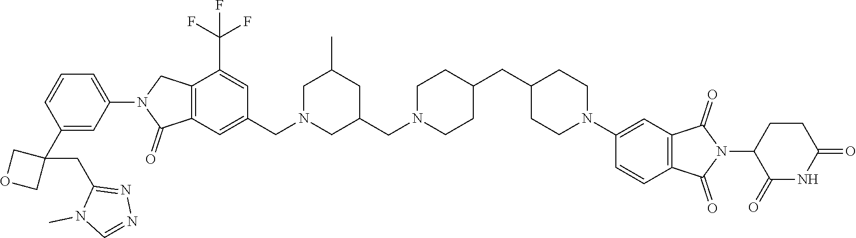 [SMILES: CC1CC(CN2CCC(CC3CCN(c4ccc5c(c4)C(=O)N(C4CCC(=O)NC4=O)C5=O)CC3)CC2)CN(Cc2cc3c(c(C(F)(F)F)c2)CN(c2cccc(C4(Cc5nncn5C)COC4)c2)C3=O)C1] | 2-(2,6-dioxopiperidin-3-yl)-5-[4-({1- [(5-methyl-1-{[2-(3-{3-[(4-methyl-4H- 1,2,4-triazol-3-yl)methyl]oxetan-3- yl}phenyl)-3-oxo-7-(trifluoromethyl)- 2,3-dihydro-1H-isoindol-5- yl]methyl}piperidin-3- yl)methyl]piperidin-4- yl}methyl)piperidin-1-yl]-2,3-dihydro- 1H-isoindole-1,3-dione | Scheme A |
| 95 | 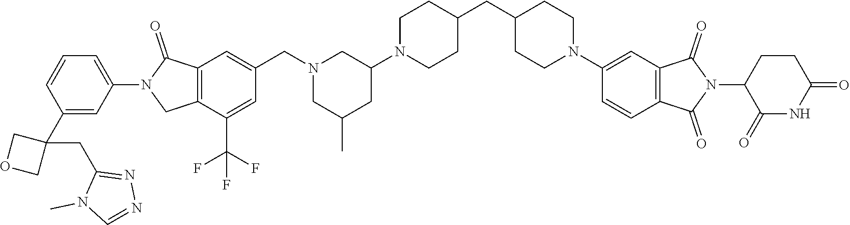 [SMILES: CC1CC(N2CCC(CC3CCN(c4ccc5c(c4)C(=O)N(C4CCC(=O)NC4=O)C5=O)CC3)CC2)CN(Cc2cc3c(c(C(F)(F)F)c2)CN(c2cccc(C4(Cc5nncn5C)COC4)c2)C3=O)C1] | 2-(2,6-dioxopiperidin-3-yl)-5-{4-[(5′- methyl-1′-{[2-(3-{3-[(4-methyl-4H- 1,2,4-triazol-3-yl)methyl]oxetan-3- yl}phenyl)-3-oxo-7-(trifluoromethyl)- 2,3-dihydro-1H-isoindol-5-yl]methyl}- [1,3′-bipiperidin]-4- yl)methyl]piperidin-1-yl}-2,3-dihydro- 1H-isoindole-1,3-dione | Scheme A |
| 96 | 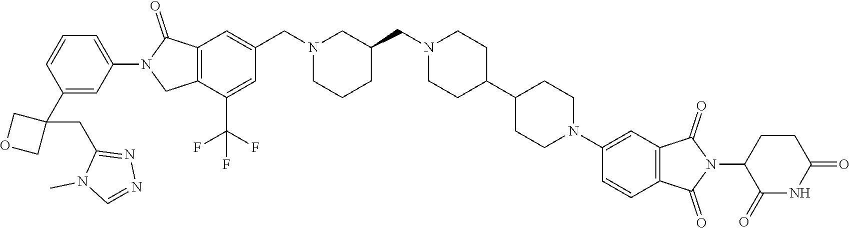 [SMILES: Cn1cnnc1CC1(c2cccc(N3Cc4c(cc(CN5CCC[C@H](CN6CCC(C7CCN(c8ccc9c(c8)C(=O)N(C8CCC(=O)NC8=O)C9=O)CC7)CC6)C5)cc4C(F)(F)F)C3=O)c2)COC1] | 2-(2,6-dioxopiperidin-3-yl)-5-(1′- {[(3R)-1-{[2-(3-{3-[(4-methyl-4H- 1,2,4-triazol-3-yl)methyl]oxetan-3- yl}phenyl)-3-oxo-7-(trifluoromethyl)- 2,3-dihydro-1H-isoindol-5- yl]methyl}piperidin-3-yl]methyl}-[4,4′- bipiperidin]-1-yl)-2,3-dihydro-1H- isoindole-1,3-dione | Scheme A |
| 97 | 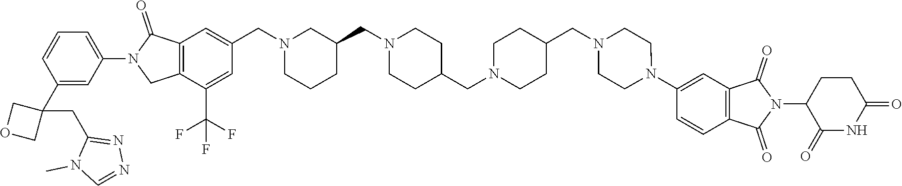 [SMILES: Cn1cnnc1CC1(c2cccc(N3Cc4c(cc(CN5CCC[C@H](CN6CCC(CN7CCC(CN8CCN(c9ccc%10c(c9)C(=O)N(C9CCC(=O)NC9=O)C%10=O)CC8)CC7)CC6)C5)cc4C(F)(F)F)C3=O)c2)COC1] | 2-(2,6-dioxopiperidin-3-yl)-5-[4-({1- [(1-{[(3R)-1-{[2-(3-{3-[(4-methyl-4H- 1,2,4-triazol-3-yl)methyl]oxetan-3- yl}phenyl)-3-oxo-7-(trifluoromethyl)- 2,3-dihydro-1H-isoindol-5- yl]methyl}piperidin-3- yl]methyl}piperidin-4- yl)methyl]piperidin-4- yl}methyl)piperazin-1-yl]-2,3-dihydro- 1H-isoindole-1,3-dione | Scheme A |
| 98 | 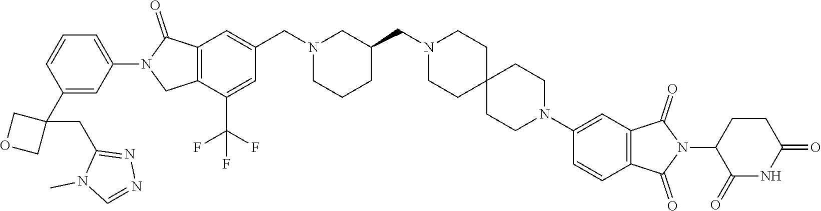 [SMILES: Cn1cnnc1CC1(c2cccc(N3Cc4c(cc(CN5CCC[C@H](CN6CCC7(CC6)CCN(c6ccc8c(c6)C(=O)N(C6CCC(=O)NC6=O)C8=O)CC7)C5)cc4C(F)(F)F)C3=O)c2)COC1] | 2-(2,6-dioxopiperidin-3-yl)-5-(9- {[(3R)-1-{[2-(3-{3-[(4-methyl-4H- 1,2,4-triazol-3-yl)methyl]oxetan-3- yl}phenyl)-3-oxo-7-(trifluoromethyl)- 2,3-dihydro-1H-isoindol-5- yl]methyl}piperidin-3-yl]methyl}-3,9- diazaspiro[5.5]undecan-3-yl)-2,3- dihydro-1H-isoindole-1,3-dione | Scheme A |
| 99 | 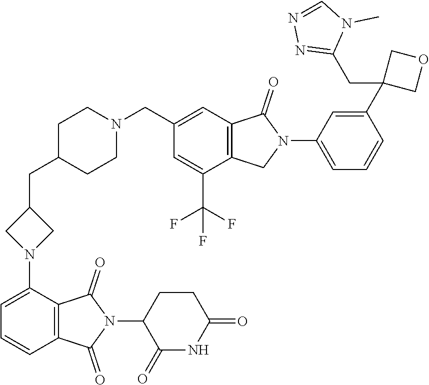 [SMILES: Cn1cnnc1CC1(c2cccc(N3Cc4c(cc(CN5CCC(CC6CN(c7cccc8c7C(=O)N(C7CCC(=O)NC7=O)C8=O)C6)CC5)cc4C(F)(F)F)C3=O)c2)COC1] | 2-(2,6-dioxopiperidin-3-yl)-4-{3-[(1- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}piperidin-4- yl)methyl]azetidin-1-yl}-2,3-dihydro- 1H-isoindole-1,3-dione | Scheme A |
| 100 | 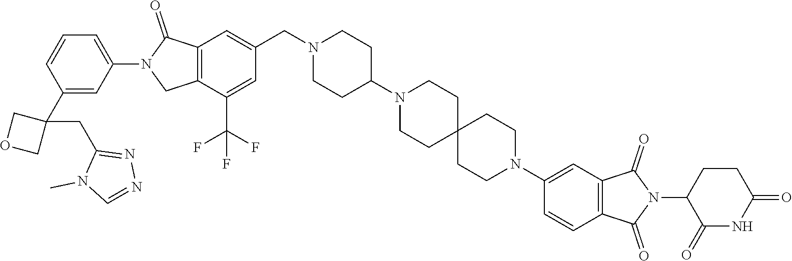 [SMILES: Cn1cnnc1CC1(c2cccc(N3Cc4c(cc(CN5CCC(N6CCC7(CCN(c8ccc9c(c8)C(=O)N(C8CCC(=O)NC8=O)C9=O)CC7)CC6)CC5)cc4C(F)(F)F)C3=O)c2)COC1] | 2-(2,6-dioxopiperidin-3-yl)-5-[9-(1- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}piperidin-4- yl)-3,9-diazaspiro[5.5]undecan-3-yl]- 2,3-dihydro-1H-isoindole-1,3-dione | Scheme A |
| 101 | 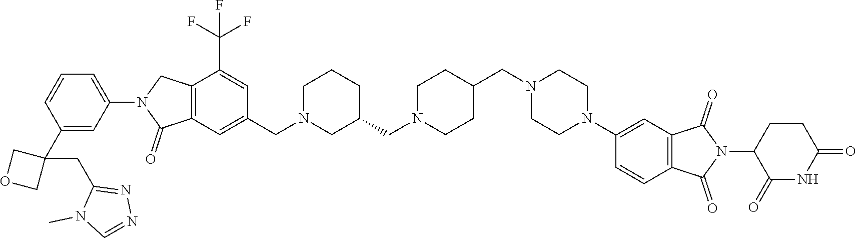 [SMILES: Cn1cnnc1CC1(c2cccc(N3Cc4c(cc(CN5CCC[C@H](CN6CCC(CN7CCN(c8ccc9c(c8)C(=O)N(C8CCC(=O)NC8=O)C9=O)CC7)CC6)C5)cc4C(F)(F)F)C3=O)c2)COC1] | 2-(2,6-dioxopiperidin-3-yl)-5-{4-[(1- {[(3R)-1-{[2-(3-{3-[(4-methyl-4H- 1,2,4-triazol-3-yl)methyl]oxetan-3- yl}phenyl)-3-oxo-7-(trifluoromethyl)- 2,3-dihydro-1H-isoindol-5- yl]methyl}piperidin-3- yl]methyl}piperidin-4- yl)methyl]piperazin-1-yl}-2,3-dihydro- 1H-isoindole-1,3-dione | Scheme A |
| 102 | 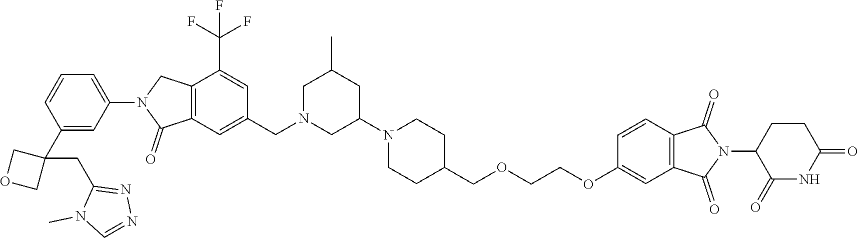 [SMILES: CC1CC(N2CCC(COCCOc3ccc4c(c3)C(=O)N(C3CCC(=O)NC3=O)C4=O)CC2)CN(Cc2cc3c(c(C(F)(F)F)c2)CN(c2cccc(C4(Cc5nncn5C)COC4)c2)C3=O)C1] | 2-(2,6-dioxopiperidin-3-yl)-5-{2-[(5′- methyl-1′-{[2-(3-{3-[(4-methyl-4H- 1,2,4-triazol-3-yl)methyl]oxetan-3- yl}phenyl)-3-oxo-7-(trifluoromethyl)- 2,3-dihydro-1H-isoindol-5-yl]methyl}- [1,3′-bipiperidin]-4- yl)methoxy]ethoxy}-2,3-dihydro-1H- isoindole-1,3-dione | Scheme A |
| 103 | 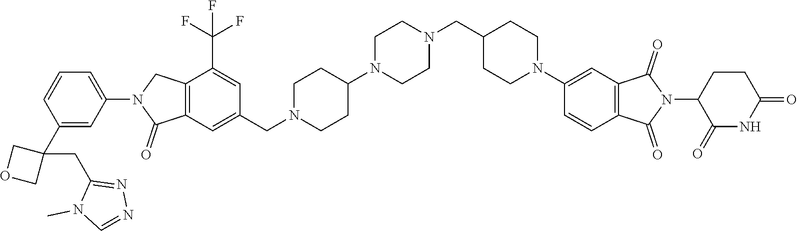 [SMILES: Cn1cnnc1CC1(c2cccc(N3Cc4c(cc(CN5CCC(N6CCN(CC7CCN(c8ccc9c(c8)C(=O)N(C8CCC(=O)NC8=O)C9=O)CC7)CC6)CC5)cc4C(F)(F)F)C3=O)c2)COC1] | 2-(2,6-dioxopiperidin-3-yl)-5-(4-{[4- (1-{[2-(3-{3-[(4-methyl-4H-1,2,4- triazol-3-yl)methyl]oxetan-3- yl}phenyl)-3-oxo-7-(trifluoromethyl)- 2,3-dihydro-1H-isoindol-5- yl]methyl}piperidin-4-yl)piperazin-1- yl]methyl}piperidin-1-yl)-2,3-dihydro- 1H-isoindole-1,3-dione | Scheme A |
| 104 | 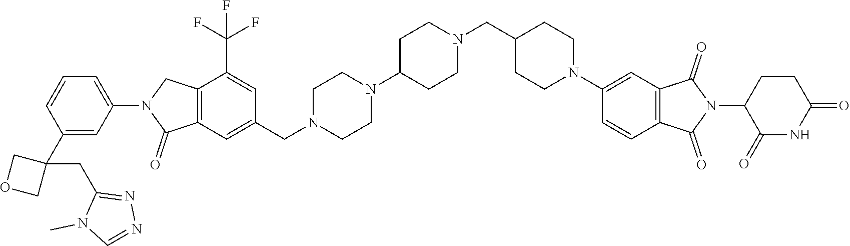 [SMILES: Cn1cnnc1CC1(c2cccc(N3Cc4c(cc(CN5CCN(C6CCN(CC7CCN(c8ccc9c(c8)C(=O)N(C8CCC(=O)NC8=O)C9=O)CC7)CC6)CC5)cc4C(F)(F)F)C3=O)c2)COC1] | 2-(2,6-dioxopiperidin-3-yl)-5-(4-{[4- (4-{[2-(3-{3-[(4-methyl-4H-1,2,4- triazol-3-yl)methyl]oxetan-3- yl}phenyl)-3-oxo-7-(trifluoromethyl)- 2,3-dihydro-1H-isoindol-5- yl]methyl}piperazin-1-yl)piperidin-1- yl]methyl}piperidin-1-yl)-2,3-dihydro- 1H-isoindole-1,3-dione | Scheme A |
| 105 | 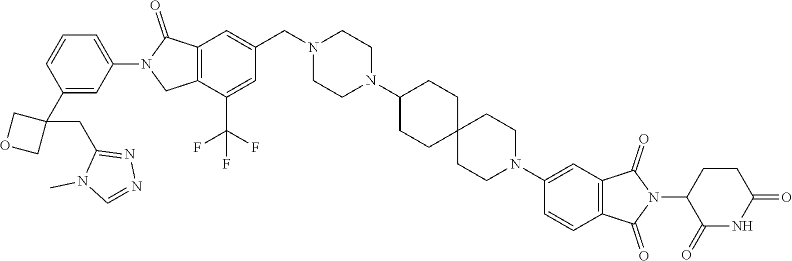 [SMILES: Cn1cnnc1CC1(c2cccc(N3Cc4c(cc(CN5CCN(C6CCC7(CC6)CCN(c6ccc8c(c6)C(=O)N(C6CCC(=O)NC6=O)C8=O)CC7)CC5)cc4C(F)(F)F)C3=O)c2)COC1] | 2-(2,6-dioxopiperidin-3-yl)-5-[9-(4- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}piperazin-1- yl)-3-azaspiro[5.5]undecan-3-yl]-2,3- dihydro-1H-isoindole-1,3-dione | Scheme A |
| 106 | 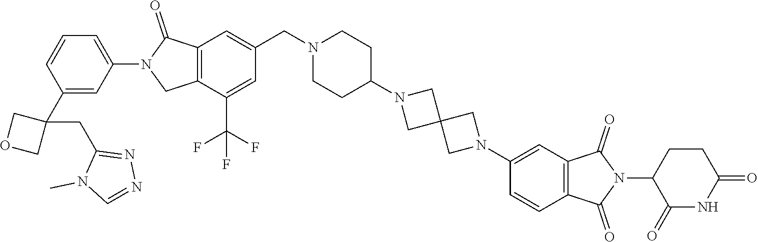 [SMILES: Cn1cnnc1CC1(c2cccc(N3Cc4c(cc(CN5CCC(N6CC7(CN(c8ccc9c(c8)C(=O)N(C8CCC(=O)NC8=O)C9=O)C7)C6)CC5)cc4C(F)(F)F)C3=O)c2)COC1] | 2-(2,6-dioxopiperidin-3-yl)-5-[6-(1- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}piperidin-4- yl)-2,6-diazaspiro[3.3]heptan-2-yl]-2,3- dihydro-1H-isoindole-1,3-dione | Scheme A |
| 107 | 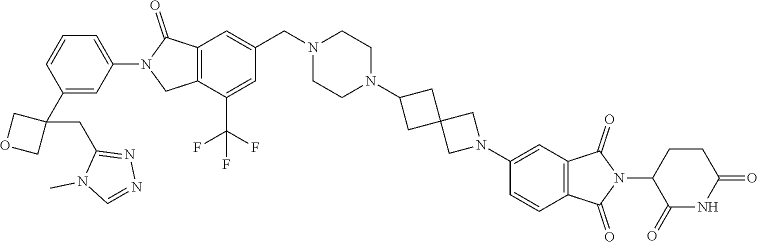 [SMILES: Cn1cnnc1CC1(c2cccc(N3Cc4c(cc(CN5CCN(C6CC7(C6)CN(c6ccc8c(c6)C(=O)N(C6CCC(=O)NC6=O)C8=O)C7)CC5)cc4C(F)(F)F)C3=O)c2)COC1] | 2-(2,6-dioxopiperidin-3-yl)-5-[6-(4- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}piperazin-1- yl)-2-azaspiro[3.3]heptan-2-yl]-2,3- dihydro-1H-isoindole-1,3-dione | Scheme A |
| 108 | 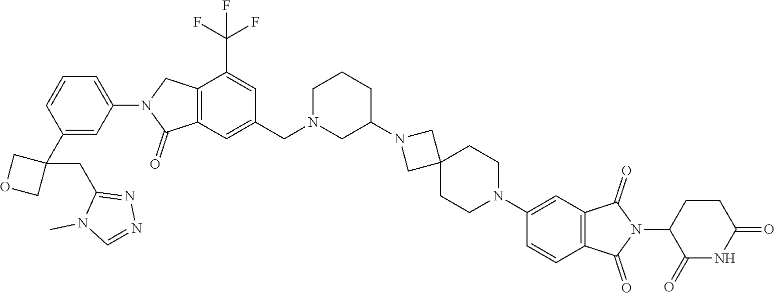 [SMILES: Cn1cnnc1CC1(c2cccc(N3Cc4c(cc(CN5CCCC(N6CC7(CCN(c8ccc9c(c8)C(=O)N(C8CCC(=O)NC8=O)C9=O)CC7)C6)C5)cc4C(F)(F)F)C3=O)c2)COC1] | 2-(2,6-dioxopiperidin-3-yl)-5-[2-(1- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}piperidin-3- yl)-2,7-diazaspiro[3.5]nonan-7-yl]-2,3- dihydro-1H-isoindole-1,3-dione | Scheme A |
| 109 | 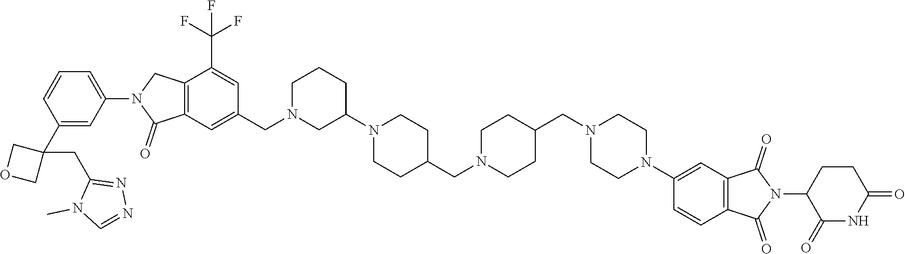 [SMILES: Cn1cnnc1CC1(c2cccc(N3Cc4c(cc(CN5CCCC(N6CCC(CN7CCC(CN8CCN(c9ccc%10c(c9)C(=O)N(C9CCC(=O)NC9=O)C%10=O)CC8)CC7)CC6)C5)cc4C(F)(F)F)C3=O)c2)COC1] | 2-(2,6-dioxopiperidin-3-yl)-5-[4-({1- [(1′-{[2-(3-{3-[(4-methyl-4H-1,2,4- triazol-3-yl)methyl]oxetan-3- yl}phenyl)-3-oxo-7-(trifluoromethyl)- 2,3-dihydro-1H-isoindol-5-yl]methyl}- [1,3′-bipiperidin]-4- yl)methyl]piperidin-4- yl}methyl)piperazin-1-yl]-2,3-dihydro- 1H-isoindole-1,3-dione | Scheme A |
| 110 | 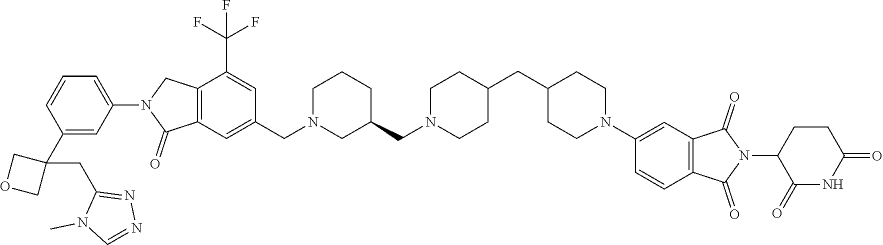 [SMILES: Cn1cnnc1CC1(c2cccc(N3Cc4c(cc(CN5CCC[C@@H](CN6CCC(CC7CCN(c8ccc9c(c8)C(=O)N(C8CCC(=O)NC8=O)C9=O)CC7)CC6)C5)cc4C(F)(F)F)C3=O)c2)COC1] | 2-(2,6-dioxopiperidin-3-yl)-5-{4-[(1- {[(3S)-1-{[2-(3-{3-[(4-methyl-4H- 1,2,4-triazol-3-yl)methyl]oxetan-3- yl}phenyl)-3-oxo-7-(trifluoromethyl)- 2,3-dihydro-1H-isoindol-5- yl]methyl}piperidin-3- yl]methyl}piperidin-4- yl)methyl]piperidin-1-yl}-2,3-dihydro- 1H-isoindole-1,3-dione | Scheme A |
| 111 | 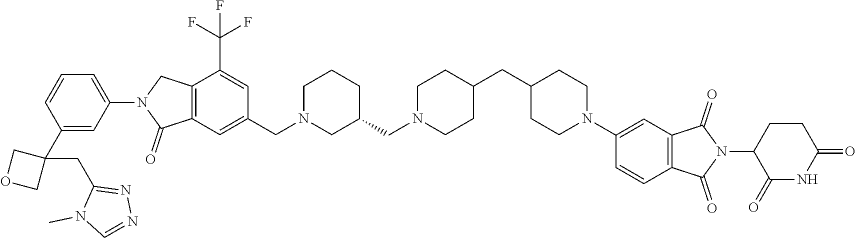 [SMILES: Cn1cnnc1CC1(c2cccc(N3Cc4c(cc(CN5CCC[C@H](CN6CCC(CC7CCN(c8ccc9c(c8)C(=O)N(C8CCC(=O)NC8=O)C9=O)CC7)CC6)C5)cc4C(F)(F)F)C3=O)c2)COC1] | 2-(2,6-dioxopiperidin-3-yl)-5-{4-[(1- {[(3R)-1-{[2-(3-{3-[(4-methyl-4H- 1,2,4-triazol-3-yl)methyl]oxetan-3- yl}phenyl)-3-oxo-7-(trifluoromethyl)- 2,3-dihydro-1H-isoindol-5- yl]methyl}piperidin-3- yl]methyl}piperidin-4- yl)methyl]piperidin-1-yl}-2,3-dihydro- 1H-isoindole-1,3-dione | Scheme A |
| 112 | 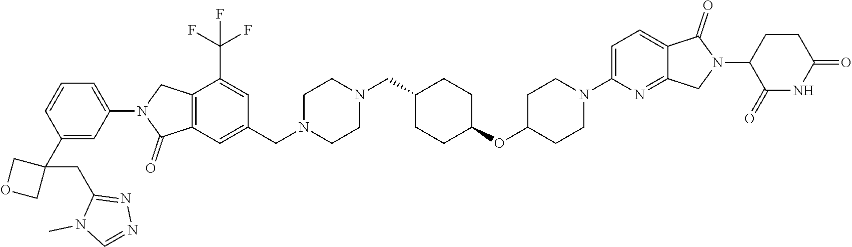 [SMILES: Cn1cnnc1CC1(c2cccc(N3Cc4c(cc(CN5CCN(C[C@H]6CC[C@H](OC7CCN(c8ccc9c(n8)CN(C8CCC(=O)NC8=O)C9=O)CC7)CC6)CC5)cc4C(F)(F)F)C3=O)c2)COC1] | 3-[5-oxo-2-(4-{[(1r,4r)-4-[(4-{[2-(3- {3-[(4-methyl-4H-1,2,4-triazol-3- yl)methyl]oxetan-3-yl}phenyl)-3-oxo- 7-(trifluoromethyl)-2,3-dihydro-1H- isoindol-5-yl]methyl}piperazin-1- yl)methyl]cyclohexyl]oxy}piperidin-1- yl)-5H,6H,7H-pyrrolo[3,4-b]pyridin-6- yl]piperidine-2,6-dione | Scheme A |
| 113 | 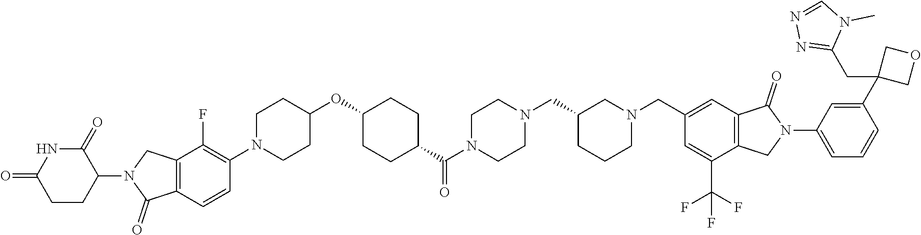 [SMILES: Cn1cnnc1CC1(c2cccc(N3Cc4c(cc(CN5CCC[C@H](CN6CCN(C(=O)[C@H]7CC[C@@H](OC8CCN(c9ccc%10c(c9F)CN(C9CCC(=O)NC9=O)C%10=O)CC8)CC7)CC6)C5)cc4C(F)(F)F)C3=O)c2)COC1] | 3-[4-fluoro-1-oxo-5-(4-{[(1r,4r)-4-(4- {[(3S)-1-{[2-(3-{3-[(4-methyl-4H- 1,2,4-triazol-3-yl)methyl]oxetan-3- yl}phenyl)-3-oxo-7-(trifluoromethyl)- 2,3-dihydro-1H-isoindol-5- yl]methyl}piperidin-3- yl]methyl}piperazine-1- carbonyl)cyclohexyl]oxy}piperidin-1- yl)-2,3-dihydro-1H-isoindol-2- yl]piperidine-2,6-dione | Scheme A |
| 114 | 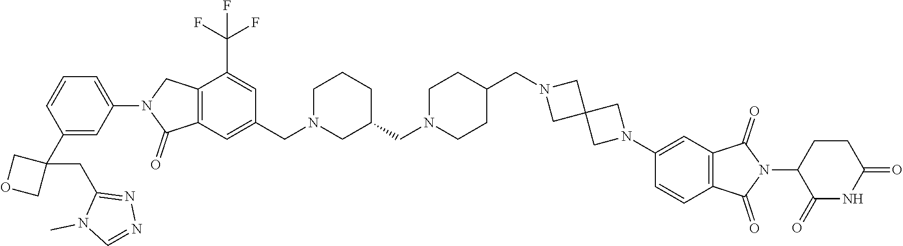 [SMILES: Cn1cnnc1CC1(c2cccc(N3Cc4c(cc(CN5CCC[C@H](CN6CCC(CN7CC8(C7)CN(c7ccc9c(c7)C(=O)N(C7CCC(=O)NC7=O)C9=O)C8)CC6)C5)cc4C(F)(F)F)C3=O)c2)COC1] | 2-(2,6-dioxopiperidin-3-yl)-5-{6-[(1- {[(3R)-1-{[2-(3-{3-[(4-methyl-4H- 1,2,4-triazol-3-yl)methyl]oxetan-3- yl}phenyl)-3-oxo-7-(trifluoromethyl)- 2,3-dihydro-1H-isoindol-5- yl]methyl}piperidin-3- yl]methyl}piperidin-4-yl)methyl]-2,6- diazaspiro[3.3]heptan-2-yl}-2,3- dihydro-1H-isoindole-1,3-dione | Scheme A |
| 115 | 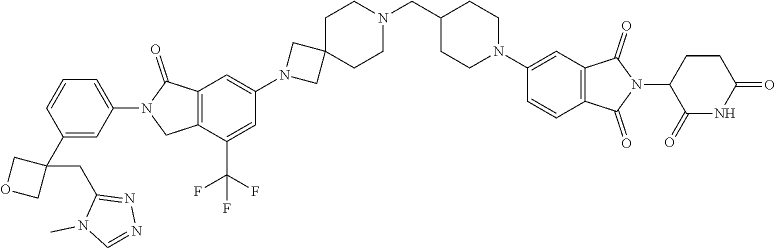 [SMILES: Cn1cnnc1CC1(c2cccc(N3Cc4c(cc(N5CC6(CCN(CC7CCN(c8ccc9c(c8)C(=O)N(C8CCC(=O)NC8=O)C9=O)CC7)CC6)C5)cc4C(F)(F)F)C3=O)c2)COC1] | 2-(2,6-dioxopiperidin-3-yl)-5-[4-({2- [2-(3-{3-[(4-methyl-4H-1,2,4-triazol-3- yl)methyl]oxetan-3-yl}phenyl)-3-oxo- 7-(trifluoromethyl)-2,3-dihydro-1H- isoindol-5-yl]-2,7- diazaspiro[3.5]nonan-7- yl}methyl)piperidin-1-yl]-2,3-dihydro- 1H-isoindole-1,3-dione | Scheme B |
| 116 | 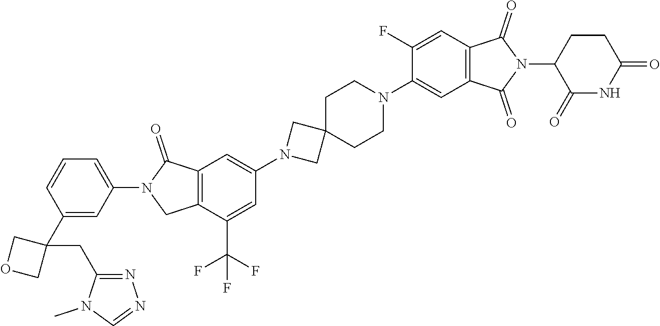 [SMILES: Cn1cnnc1CC1(c2cccc(N3Cc4c(cc(N5CC6(CCN(c7cc8c(cc7F)C(=O)N(C7CCC(=O)NC7=O)C8=O)CC6)C5)cc4C(F)(F)F)C3=O)c2)COC1] | 2-(2,6-dioxopiperidin-3-yl)-5-fluoro-6- {2-[2-(3-{3-[(4-methyl-4H-1,2,4- triazol-3-yl)methyl]oxetan-3- yl}phenyl)-3-oxo-7-(trifluoromethyl)- 2,3-dihydro-1H-isoindol-5-yl]-2,7- diazaspiro[3.5]nonan-7-yl}-2,3- dihydro-1H-isoindole-1,3-dione | Scheme B |
| 117 | 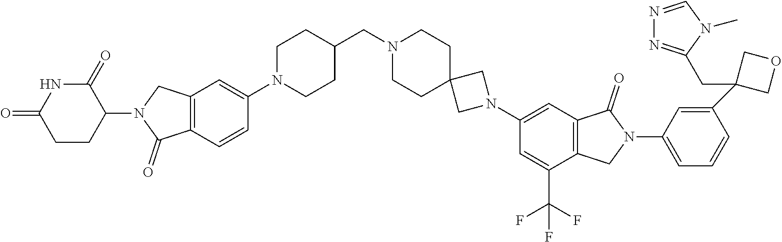 [SMILES: Cn1cnnc1CC1(c2cccc(N3Cc4c(cc(N5CC6(CCN(CC7CCN(c8ccc9c(c8)CN(C8CCC(=O)NC8=O)C9=O)CC7)CC6)C5)cc4C(F)(F)F)C3=O)c2)COC1] | 3-{5-[4-({2-[2-(3-{3-[(4-methyl-4H- 1,2,4-triazol-3-yl)methyl]oxetan-3- yl}phenyl)-3-oxo-7-(trifluoromethyl)- 2,3-dihydro-1H-isoindol-5-yl]-2,7- diazaspiro[3.5]nonan-7- yl}methyl)piperidin-1-yl]-1-oxo-2,3- dihydro-1H-isoindol-2-yl}piperidine- 2,6-dione | Scheme B |
| 118 | 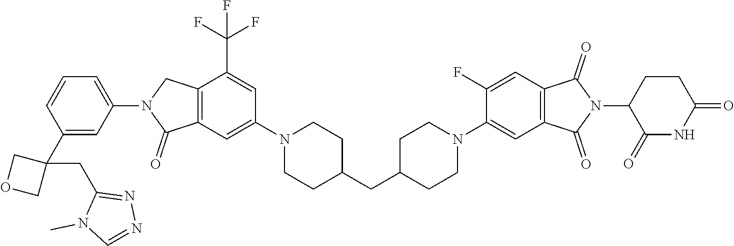 [SMILES: Cn1cnnc1CC1(c2cccc(N3Cc4c(cc(N5CCC(CC6CCN(c7cc8c(cc7F)C(=O)N(C7CCC(=O)NC7=O)C8=O)CC6)CC5)cc4C(F)(F)F)C3=O)c2)COC1] | 2-(2,6-dioxopiperidin-3-yl)-5-fluoro-6- [4-({1-[2-(3-{3-[(4-methyl-4H-1,2,4- triazol-3-yl)methyl]oxetan-3- yl}phenyl)-3-oxo-7-(trifluoromethyl)- 2,3-dihydro-1H-isoindol-5- yl]piperidin-4-yl}methyl)piperidin-1- yl]-2,3-dihydro-1H-isoindole-1,3-dione | Scheme B |
| 119 | 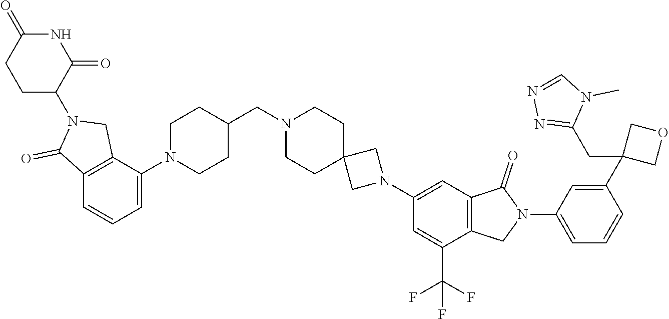 [SMILES: Cn1cnnc1CC1(c2cccc(N3Cc4c(cc(N5CC6(CCN(CC7CCN(c8cccc9c8CN(C8CCC(=O)NC8=O)C9=O)CC7)CC6)C5)cc4C(F)(F)F)C3=O)c2)COC1] | 3-{4-[4-({2-[2-(3-{3-[(4-methyl-4H- 1,2,4-triazol-3-yl)methyl]oxetan-3- yl}phenyl)-3-oxo-7-(trifluoromethyl)- 2,3-dihydro-1H-isoindol-5-yl]-2,7- diazaspiro[3.5]nonan-7- yl}methyl)piperidin-1-yl]-1-oxo-2,3- dihydro-1H-isoindol-2-yl}piperidine- 2,6-dione | Scheme B |
| 120 | 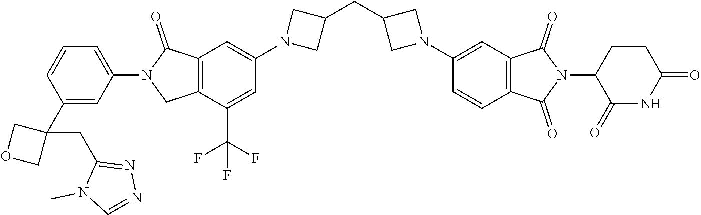 [SMILES: Cn1cnnc1CC1(c2cccc(N3Cc4c(cc(N5CC(CC6CN(c7ccc8c(c7)C(=O)N(C7CCC(=O)NC7=O)C8=O)C6)C5)cc4C(F)(F)F)C3=O)c2)COC1] | 2-(2,6-dioxopiperidin-3-yl)-5-[3-({1- [2-(3-{3-[(4-methyl-4H-1,2,4-triazol-3- yl)methyl]oxetan-3-yl}phenyl)-3-oxo- 7-(trifluoromethyl)-2,3-dihydro-1H- isoindol-5-yl]azetidin-3- yl}methyl)azetidin-1-yl]-2,3-dihydro- 1H-isoindole-1,3-dione | Scheme B |
| 121 | 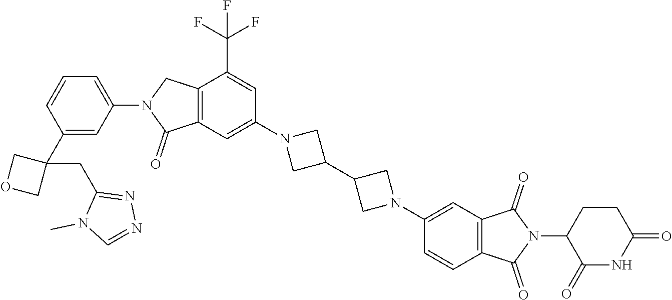 [SMILES: Cn1cnnc1CC1(c2cccc(N3Cc4c(cc(N5CC(C6CN(c7ccc8c(c7)C(=O)N(C7CCC(=O)NC7=O)C8=O)C6)C5)cc4C(F)(F)F)C3=O)c2)COC1] | 2-(2,6-dioxopiperidin-3-yl)-5-{1′-[2-(3- {3-[(4-methyl-4H-1,2,4-triazol-3- yl)methyl]oxetan-3-yl}phenyl)-3-oxo- 7-(trifluoromethyl)-2,3-dihydro-1H- isoindol-5-yl]-[3,3′-biazetidin]-1-yl}- 2,3-dihydro-1H-isoindole-1,3-dione | Scheme B |
| 122 | 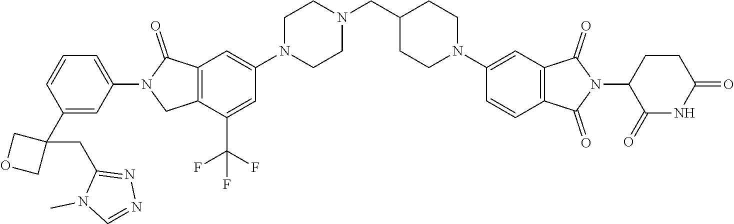 [SMILES: Cn1cnnc1CC1(c2cccc(N3Cc4c(cc(N5CCN(CC6CCN(c7ccc8c(c7)C(=O)N(C7CCC(=O)NC7=O)C8=O)CC6)CC5)cc4C(F)(F)F)C3=O)c2)COC1] | 2-(2,6-dioxopiperidin-3-yl)-5-[4-({4- [2-(3-{3-[(4-methyl-4H-1,2,4-triazol-3- yl)methyl]oxetan-3-yl}phenyl)-3-oxo- 7-(trifluoromethyl)-2,3-dihydro-1H- isoindol-5-yl]piperazin-1- yl}methyl)piperidin-1-yl]-2,3-dihydro- 1H-isoindole-1,3-dione | Scheme B |
| 123 | 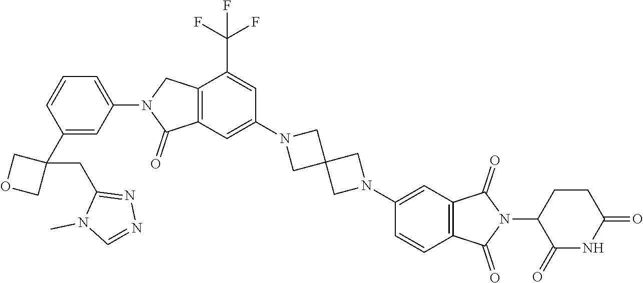 [SMILES: Cn1cnnc1CC1(c2cccc(N3Cc4c(cc(N5CC6(CN(c7ccc8c(c7)C(=O)N(C7CCC(=O)NC7=O)C8=O)C6)C5)cc4C(F)(F)F)C3=O)c2)COC1] | 2-(2,6-dioxopiperidin-3-yl)-5-{6-[2-(3- {3-[(4-methyl-4H-1,2,4-triazol-3- yl)methyl]oxetan-3-yl}phenyl)-3-oxo- 7-(trifluoromethyl)-2,3-dihydro-1H- isoindol-5-yl]-2,6- diazaspiro[3.3]heptan-2-yl}-2,3- dihydro-1H-isoindole-1,3-dione | Scheme B |
| 124 | 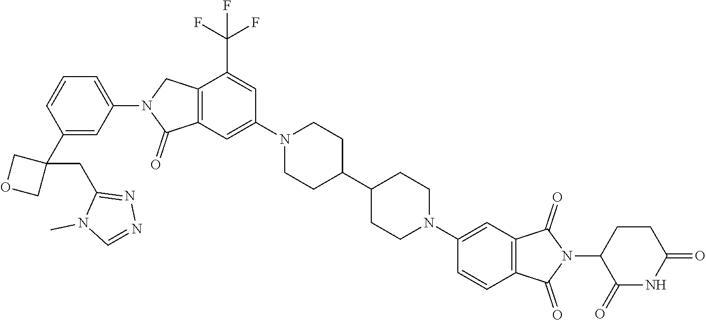 [SMILES: Cn1cnnc1CC1(c2cccc(N3Cc4c(cc(N5CCC(C6CCN(c7ccc8c(c7)C(=O)N(C7CCC(=O)NC7=O)C8=O)CC6)CC5)cc4C(F)(F)F)C3=O)c2)COC1] | 2-(2,6-dioxopiperidin-3-yl)-5-{1′-[2-(3- {3-[(4-methyl-4H-1,2,4-triazol-3- yl)methyl]oxetan-3-yl}phenyl)-3-oxo- 7-(trifluoromethyl)-2,3-dihydro-1H- isoindol-5-yl]-[4,4′-bipiperidin]-1-yl}- 2,3-dihydro-1H-isoindole-1,3-dione | Scheme B |
| 125 | 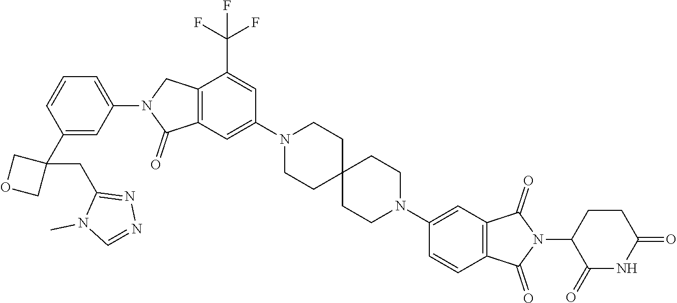 [SMILES: Cn1cnnc1CC1(c2cccc(N3Cc4c(cc(N5CCC6(CCN(c7ccc8c(c7)C(=O)N(C7CCC(=O)NC7=O)C8=O)CC6)CC5)cc4C(F)(F)F)C3=O)c2)COC1] | 2-(2,6-dioxopiperidin-3-yl)-5-{9-[2-(3- {3-[(4-methyl-4H-1,2,4-triazol-3- yl)methyl]oxetan-3-yl}phenyl)-3-oxo- 7-(trifluoromethyl)-2,3-dihydro-1H- isoindol-5-yl]-3,9- diazaspiro[5.5]undecan-3-yl}-2,3- dihydro-1H-isoindole-1,3-dione | Scheme B |
| 126 | 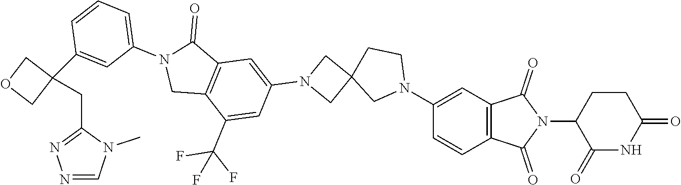 [SMILES: Cn1cnnc1CC1(c2cccc(N3Cc4c(cc(N5CC6(CCN(c7ccc8c(c7)C(=O)N(C7CCC(=O)NC7=O)C8=O)C6)C5)cc4C(F)(F)F)C3=O)c2)COC1] | 2-(2,6-dioxopiperidin-3-yl)-5-{2-[2-(3- {3-[(4-methyl-4H-1,2,4-triazol-3- yl)methyl]oxetan-3-yl}phenyl)-3-oxo- 7-(trifluoromethyl)-2,3-dihydro-1H- isoindol-5-yl]-2,6-diazaspiro[3.4]octan- 6-yl}-2,3-dihydro-1H-isoindole-1,3- dione | Scheme B |
| 127 | 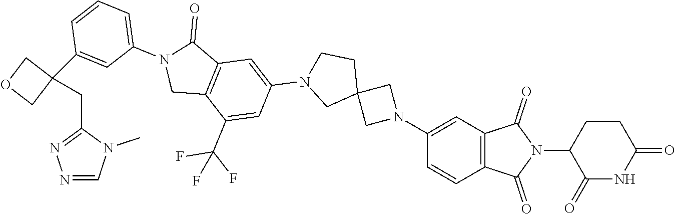 [SMILES: Cn1cnnc1CC1(c2cccc(N3Cc4c(cc(N5CCC6(C5)CN(c5ccc7c(c5)C(=O)N(C5CCC(=O)NC5=O)C7=O)C6)cc4C(F)(F)F)C3=O)c2)COC1] | 2-(2,6-dioxopiperidin-3-yl)-5-{6-[2-(3- {3-[(4-methyl-4H-1,2,4-triazol-3- yl)methyl]oxetan-3-yl}phenyl)-3-oxo- 7-(trifluoromethyl)-2,3-dihydro-1H- isoindol-5-yl]-2,6-diazaspiro[3.4]octan- 2-yl}-2,3-dihydro-1H-isoindole-1,3- dione | Scheme B |
| 128 | 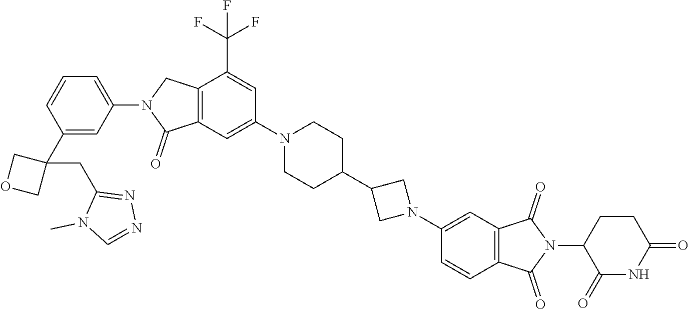 [SMILES: Cn1cnnc1CC1(c2cccc(N3Cc4c(cc(N5CCC(C6CN(c7ccc8c(c7)C(=O)N(C7CCC(=O)NC7=O)C8=O)C6)CC5)cc4C(F)(F)F)C3=O)c2)COC1] | 2-(2,6-dioxopiperidin-3-yl)-5-(3-{1-[2- (3-{3-[(4-methyl-4H-1,2,4-triazol-3- yl)methyl]oxetan-3-yl}phenyl)-3-oxo- 7-(trifluoromethyl)-2,3-dihydro-1H- isoindol-5-yl]piperidin-4-yl}azetidin-1- yl)-2,3-dihydro-1H-isoindole-1,3-dione | Scheme B |
| 129 | 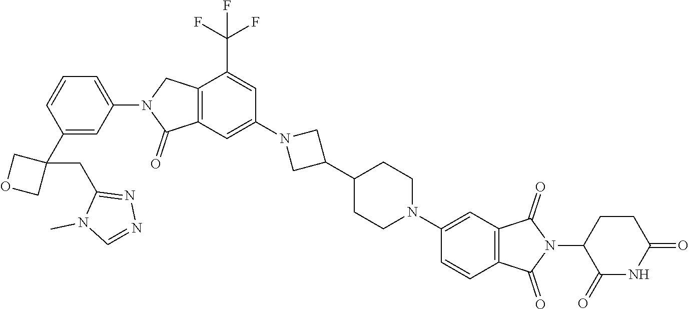 [SMILES: Cn1cnnc1CC1(c2cccc(N3Cc4c(cc(N5CC(C6CCN(c7ccc8c(c7)C(=O)N(C7CCC(=O)NC7=O)C8=O)CC6)C5)cc4C(F)(F)F)C3=O)c2)COC1] | 2-(2,6-dioxopiperidin-3-yl)-5-(4-{1-[2- (3-{3-[(4-methyl-4H-1,2,4-triazol-3- yl)methyl]oxetan-3-yl}phenyl)-3-oxo- 7-(trifluoromethyl)-2,3-dihydro-1H- isoindol-5-yl]azetidin-3-yl}piperidin-1- yl)-2,3-dihydro-1H-isoindole-1,3-dione | Scheme B |
| 130 | 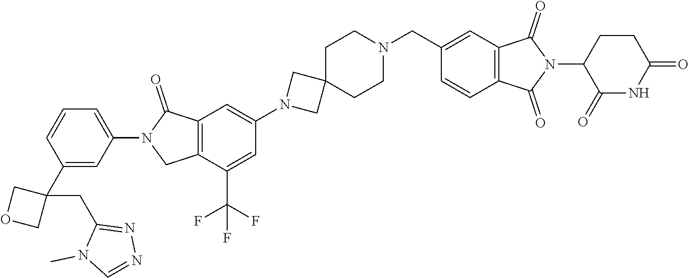 [SMILES: Cn1cnnc1CC1(c2cccc(N3Cc4c(cc(N5CC6(CCN(Cc7ccc8c(c7)C(=O)N(C7CCC(=O)NC7=O)C8=O)CC6)C5)cc4C(F)(F)F)C3=O)c2)COC1] | 2-(2,6-dioxopiperidin-3-yl)-5-({2-[2- (3-{3-[(4-methyl-4H-1,2,4-triazol-3- yl)methyl]oxetan-3-yl}phenyl)-3-oxo- 7-(trifluoromethyl)-2,3-dihydro-1H- isoindol-5-yl]-2,7- diazaspiro[3.5]nonan-7-yl}methyl)-2,3- dihydro-1H-isoindole-1,3-dione | Scheme B |
| 131 | 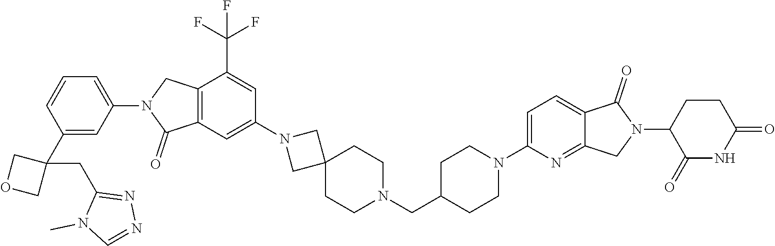 [SMILES: Cn1cnnc1CC1(c2cccc(N3Cc4c(cc(N5CC6(CCN(CC7CCN(c8ccc9c(n8)CN(C8CCC(=O)NC8=O)C9=O)CC7)CC6)C5)cc4C(F)(F)F)C3=O)c2)COC1] | 3-{2-[4-({2-[2-(3-{3-[(4-methyl-4H- 1,2,4-triazol-3-yl)methyl]oxetan-3- yl}phenyl)-3-oxo-7-(trifluoromethyl)- 2,3-dihydro-1H-isoindol-5-yl]-2,7- diazaspiro[3.5]nonan-7- yl}methyl)piperidin-1-yl]-5-oxo- 5H,6H,7H-pyrrolo[3,4-b]pyridin-6- yl}piperidine-2,6-dione | Scheme B |
| 132 | 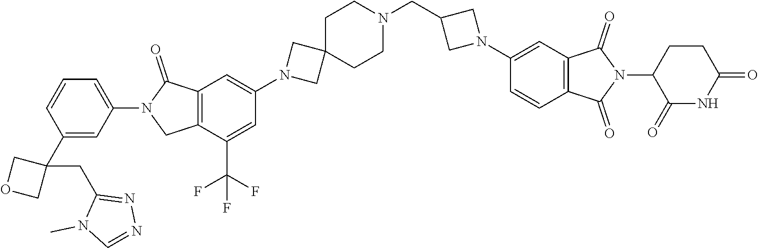 [SMILES: Cn1cnnc1CC1(c2cccc(N3Cc4c(cc(N5CC6(CCN(CC7CN(c8ccc9c(c8)C(=O)N(C8CCC(=O)NC8=O)C9=O)C7)CC6)C5)cc4C(F)(F)F)C3=O)c2)COC1] | 2-(2,6-dioxopiperidin-3-yl)-5-[3-({2- [2-(3-{3-[(4-methyl-4H-1,2,4-triazol-3- yl)methyl]oxetan-3-yl}phenyl)-3-oxo- 7-(trifluoromethyl)-2,3-dihydro-1H- isoindol-5-yl]-2,7- diazaspiro[3.5]nonan-7- yl}methyl)azetidin-1-yl]-2,3-dihydro- 1H-isoindole-1,3-dione | Scheme B |
| 133 | 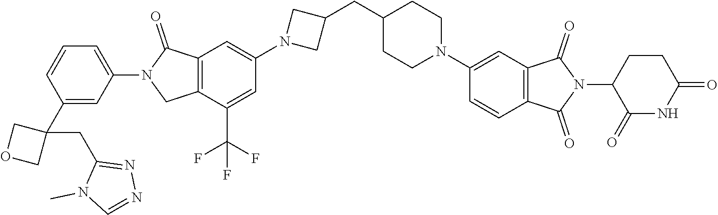 [SMILES: Cn1cnnc1CC1(c2cccc(N3Cc4c(cc(N5CC(CC6CCN(c7ccc8c(c7)C(=O)N(C7CCC(=O)NC7=O)C8=O)CC6)C5)cc4C(F)(F)F)C3=O)c2)COC1] | 2-(2,6-dioxopiperidin-3-yl)-5-[4-({1- [2-(3-{3-[(4-methyl-4H-1,2,4-triazol-3- yl)methyl]oxetan-3-yl}phenyl)-3-oxo- 7-(trifluoromethyl)-2,3-dihydro-1H- isoindol-5-yl]azetidin-3- yl}methyl)piperidin-1-yl]-2,3-dihydro- 1H-isoindole-1,3-dione | Scheme B |
| 134 | 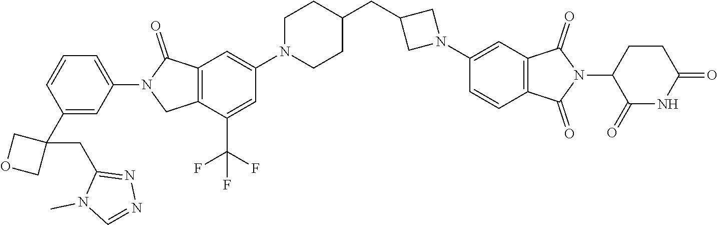 [SMILES: Cn1cnnc1CC1(c2cccc(N3Cc4c(cc(N5CCC(CC6CN(c7ccc8c(c7)C(=O)N(C7CCC(=O)NC7=O)C8=O)C6)CC5)cc4C(F)(F)F)C3=O)c2)COC1] | 2-(2,6-dioxopiperidin-3-yl)-5-[3-({1- [2-(3-{3-[(4-methyl-4H-1,2,4-triazol-3- yl)methyl]oxetan-3-yl}phenyl)-3-oxo- 7-(trifluoromethyl)-2,3-dihydro-1H- isoindol-5-yl]piperidin-4- yl}methyl)azetidin-1-yl]-2,3-dihydro- 1H-isoindole-1,3-dione | Scheme B |
| 135 | 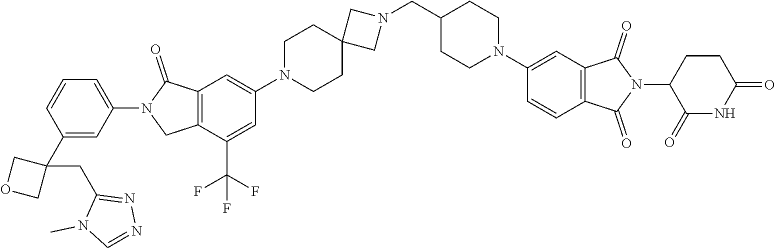 [SMILES: Cn1cnnc1CC1(c2cccc(N3Cc4c(cc(N5CCC6(CC5)CN(CC5CCN(c7ccc8c(c7)C(=O)N(C7CCC(=O)NC7=O)C8=O)CC5)C6)cc4C(F)(F)F)C3=O)c2)COC1] | 2-(2,6-dioxopiperidin-3-yl)-5-[4-({7- [2-(3-{3-[(4-methyl-4H-1,2,4-triazol-3- yl)methyl]oxetan-3-yl}phenyl)-3-oxo- 7-(trifluoromethyl)-2,3-dihydro-1H- isoindol-5-yl]-2,7- diazaspiro[3.5]nonan-2- yl}methyl)piperidin-1-yl]-2,3-dihydro- 1H-isoindole-1,3-dione | Scheme B |
| 136 | 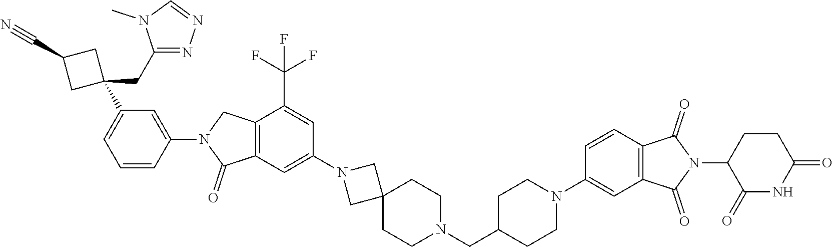 [SMILES: Cn1cnnc1C[C@]1(c2cccc(N3Cc4c(cc(N5CC6(CCN(CC7CCN(c8ccc9c(c8)C(=O)N(C8CCC(=O)NC8=O)C9=O)CC7)CC6)C5)cc4C(F)(F)F)C3=O)c2)C[C@H](C#N)C1] | (1r,3r)-3-(3-{6-[7-({1-[2-(2,6- dioxopiperidin-3-yl)-1,3-dioxo-2,3- dihydro-1H-isoindol-5-yl]piperidin-4- yl}methyl)-2,7-diazaspiro[3.5]nonan-2- yl]-1-oxo-4-(trifluoromethyl)-2,3- dihydro-1H-isoindol-2-yl}phenyl)-3- [(4-methyl-4H-1,2,4-triazol-3- yl)methyl]cyclobutane-1-carbonitrile | Scheme B |
| 137 | 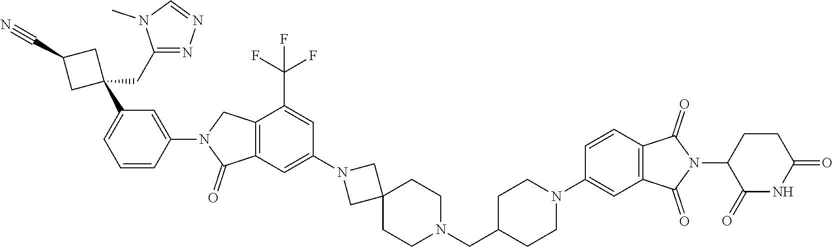 [SMILES: Cn1cnnc1C[C@]1(c2cccc(N3Cc4c(cc(N5CC6(CCN(CC7CCN(c8ccc9c(c8)C(=O)N(C8CCC(=O)NC8=O)C9=O)CC7)CC6)C5)cc4C(F)(F)F)C3=O)c2)C[C@@H](C#N)C1] | (1s,3s)-3-(3-{6-[7-({1-[2-(2,6- dioxopiperidin-3-yl)-1,3-dioxo-2,3- dihydro-1H-isoindol-5-yl]piperidin-4- yl}methyl)-2,7-diazaspiro[3.5]nonan-2- yl]-1-oxo-4-(trifluoromethyl)-2,3- dihydro-1H-isoindol-2-yl}phenyl)-3- [(4-methyl-4H-1,2,4-triazol-3- yl)methyl]cyclobutane-1-carbonitrile | Scheme B |
| 138 | 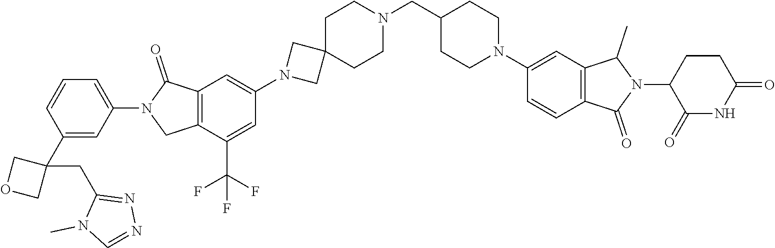 [SMILES: CC1c2cc(N3CCC(CN4CCC5(CC4)CN(c4cc6c(c(C(F)(F)F)c4)CN(c4cccc(C7(Cc8nncn8C)COC7)c4)C6=O)C5)CC3)ccc2C(=O)N1C1CCC(=O)NC1=O] | 3-{3-methyl-5-[4-({2-[2-(3-{3-[(4- methyl-4H-1,2,4-triazol-3- yl)methyl]oxetan-3-yl}phenyl)-3-oxo- 7-(trifluoromethyl)-2,3-dihydro-1H- isoindol-5-yl]-2,7- diazaspiro[3.5]nonan-7- yl}methyl)piperidin-1-yl]-1-oxo-2,3- dihydro-1H-isoindol-2-yl}piperidine- 2,6-dione | Scheme B |
| 139 | 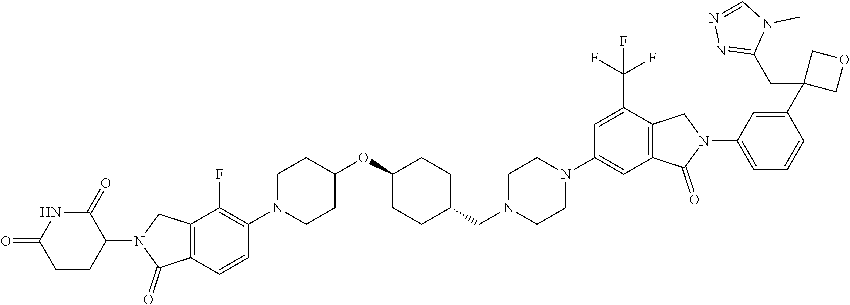 [SMILES: Cn1cnnc1CC1(c2cccc(N3Cc4c(cc(N5CCN(C[C@H]6CC[C@H](OC7CCN(c8ccc9c(c8F)CN(C8CCC(=O)NC8=O)C9=O)CC7)CC6)CC5)cc4C(F)(F)F)C3=O)c2)COC1] | 3-[4-fluoro-1-oxo-5-(4-{[(1r,4r)-4-({4- [2-(3-{3-[(4-methyl-4H-1,2,4-triazol-3- yl)methyl]oxetan-3-yl}phenyl)-3-oxo- 7-(trifluoromethyl)-2,3-dihydro-1H- isoindol-5-yl]piperazin-1- yl}methyl)cyclohexyl]oxy}piperidin-1- yl)-2,3-dihydro-1H-isoindol-2- yl]piperidine-2,6-dione | Scheme B |
| 140 | 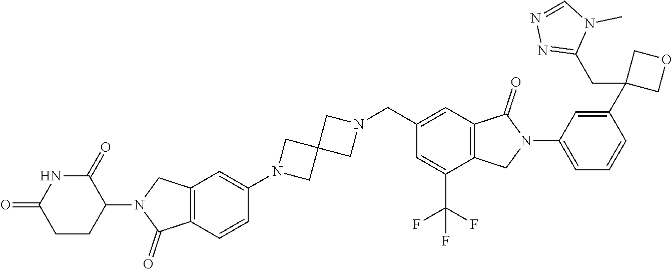 [SMILES: Cn1cnnc1CC1(c2cccc(N3Cc4c(cc(CN5CC6(C5)CN(c5ccc7c(c5)CN(C5CCC(=O)NC5=O)C7=O)C6)cc4C(F)(F)F)C3=O)c2)COC1] | 3-[5-(6-{[2-(3-{3-[(4-methyl-4H-1,2,4- triazol-3-yl)methyl]oxetan-3- yl}phenyl)-3-oxo-7-(trifluoromethyl)- 2,3-dihydro-1H-isoindol-5-yl]methyl}- 2,6-diazaspiro[3.3]heptan-2-yl)-1-oxo- 2,3-dihydro-1H-isoindol-2- yl]piperidine-2,6-dione | Scheme C |
| 141 | 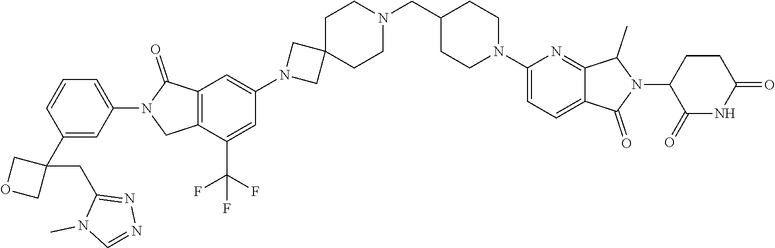 [SMILES: CC1c2nc(N3CCC(CN4CCC5(CC4)CN(c4cc6c(c(C(F)(F)F)c4)CN(c4cccc(C7(Cc8nncn8C)COC7)c4)C6=O)C5)CC3)ccc2C(=O)N1C1CCC(=O)NC1=O] | 3-{7-methyl-2-[4-({2-[2-(3-{3-[(4- methyl-4H-1,2,4-triazol-3- yl)methyl]oxetan-3-yl}phenyl)-3-oxo- 7-(trifluoromethyl)-2,3-dihydro-1H- isoindol-5-yl]-2,7- diazaspiro[3.5]nonan-7- yl}methyl)piperidin-1-yl]-5-oxo- 5H,6H,7H-pyrrolo[3,4-b]pyridin-6- yl}piperidine-2,6-dione | Scheme B |
| 142 | 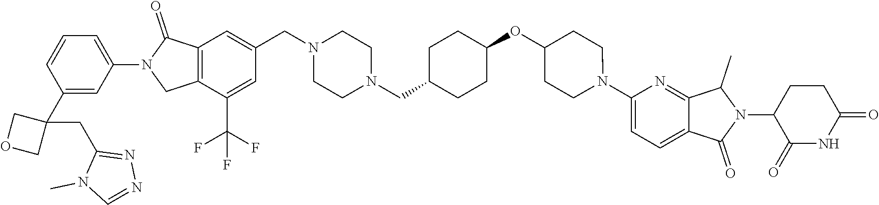 [SMILES: CC1c2nc(N3CCC(O[C@H]4CC[C@H](CN5CCN(Cc6cc7c(c(C(F)(F)F)c6)CN(c6cccc(C8(Cc9nncn9C)COC8)c6)C7=O)CC5)CC4)CC3)ccc2C(=O)N1C1CCC(=O)NC1=O] | 3-[7-methyl-5-oxo-2-(4-{[(1r,4r)-4-[(4- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}piperazin-1- yl)methyl]cyclohexyl]oxy}piperidin-1- yl)-5H,6H,7H-pyrrolo[3,4-b]pyridin-6- yl]piperidine-2,6-dione | Scheme A |
| 143 | 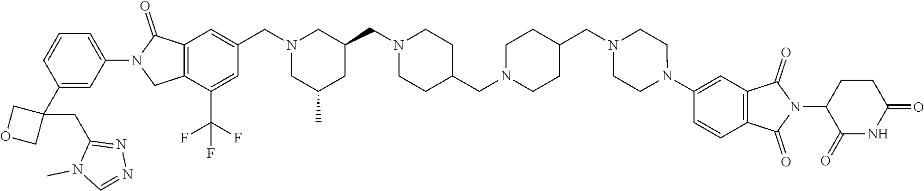 [SMILES: C[C@H]1C[C@H](CN2CCC(CN3CCC(CN4CCN(c5ccc6c(c5)C(=O)N(C5CCC(=O)NC5=O)C6=O)CC4)CC3)CC2)CN(Cc2cc3c(c(C(F)(F)F)c2)CN(c2cccc(C4(Cc5nncn5C)COC4)c2)C3=O)C1] | 2-(2,6-dioxopiperidin-3-yl)-5-[4-({1- [(1-{[(3R,5S)-5-methyl-1-{[2-(3-{3- [(4-methyl-4H-1,2,4-triazol-3- yl)methyl]oxetan-3-yl}phenyl)-3-oxo- 7-(trifluoromethyl)-2,3-dihydro-1H- isoindol-5-yl]methyl}piperidin-3- yl]methyl}piperidin-4- yl)methyl]piperidin-4- yl}methyl)piperazin-1-yl]-2,3-dihydro- 1H-isoindole-1,3-dione | Example 74 |
| 144 | 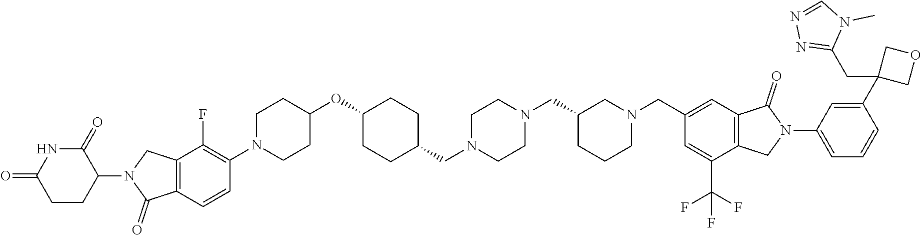 [SMILES: Cn1cnnc1CC1(c2cccc(N3Cc4c(cc(CN5CCC[C@H](CN6CCN(C[C@H]7CC[C@@H](OC8CCN(c9ccc%10c(c9F)CN(C9CCC(=O)NC9=O)C%10=O)CC8)CC7)CC6)C5)cc4C(F)(F)F)C3=O)c2)COC1] | 3-[4-fluoro-1-oxo-5-(4-{[(1r,4r)-4-[(4- {[(3S)-1-{[2-(3-{1-[(4-methyl-4H- 1,2,4-triazol-3- yl)methyl]cyclobutyl}phenyl)-3-oxo-7- (trifluoromethyl)-2,3-dihydro-1H- isoindol-5-yl]methyl}piperidin-3- yl]methyl}piperazin-1- yl)methyl]cyclohexyl]oxy}piperidin-1- yl)-2,3-dihydro-1H-isoindol-2- yl]piperidine-2,6-dione | Scheme A |
| 145 | 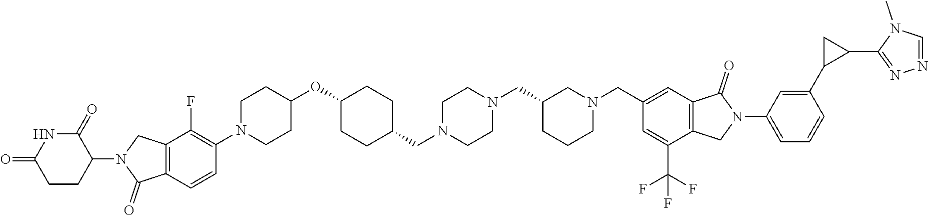 [SMILES: Cn1cnnc1C1CC1c1cccc(N2Cc3c(cc(CN4CCC[C@H](CN5CCN(C[C@H]6CC[C@@H](OC7CCN(c8ccc9c(c8F)CN(C8CCC(=O)NC8=O)C9=O)CC7)CC6)CC5)C4)cc3C(F)(F)F)C2=O)c1] | 3-[4-fluoro-1-oxo-5-(4-{[(1r,4r)-4-[(4- {[(3S)-1-[(2-{3-[2-(4-methyl-4H-1,2,4- triazol-3-yl)cyclopropyl]phenyl}-3- oxo-7-(trifluoromethyl)-2,3-dihydro- 1H-isoindol-5-yl)methyl]piperidin-3- yl]methyl}piperazin-1- yl)methyl]cyclohexyl]oxy}piperidin-1- yl)-2,3-dihydro-1H-isoindol-2- yl]piperidine-2,6-dione | Included |
| 156 | 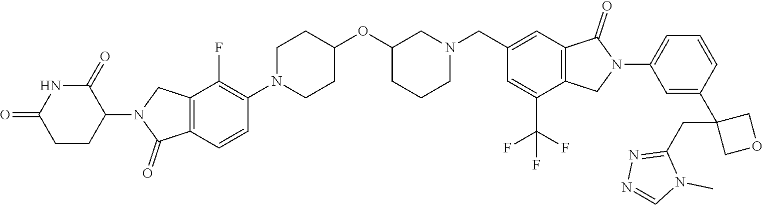 [SMILES: Cn1cnnc1CC1(c2cccc(N3Cc4c(cc(CN5CCCC(OC6CCN(c7ccc8c(c7F)CN(C7CCC(=O)NC7=O)C8=O)CC6)C5)cc4C(F)(F)F)C3=O)c2)COC1] | 3-(4-fluoro-5-{4-[(1-{[2-(3-{3-[(4- methyl-4H-1,2,4-triazol-3- yl)methyl]oxetan-3-yl}phenyl)-3-oxo- 7-(trifluoromethyl)-2,3-dihydro-1H- isoindol-5-yl]methyl}piperidin-3- yl)oxy]piperidin-1-yl}-1-oxo-2,3- dihydro-1H-isoindol-2-yl)piperidine- 2,6-dione | Scheme A |
| 157 | 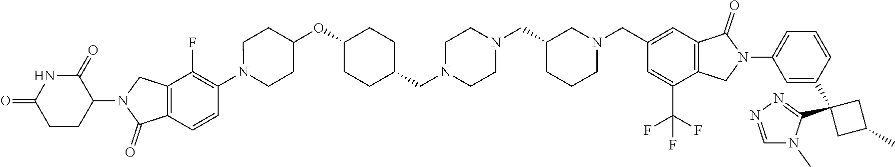 [SMILES: Cn1cnnc1[C@]1(c2cccc(N3Cc4c(cc(CN5CCC[C@H](CN6CCN(C[C@H]7CC[C@@H](OC8CCN(c9ccc%10c(c9F)CN(C9CCC(=O)NC9=O)C%10=O)CC8)CC7)CC6)C5)cc4C(F)(F)F)C3=O)c2)C[C@@H](C)C1] | 3-[4-fluoro-1-oxo-5-(4-{[(1r,4r)-4-[(4- {[(3S)-1-[(3-oxo-2-{3-[(1r,3s)-3- methyl-1-(4-methyl-4H-1,2,4-triazol-3- yl)cyclobutyl]phenyl}-7- (trifluoromethyl)-2,3-dihydro-1H- isoindol-5-yl)methyl]piperidin-3- yl]methyl}piperazin-1- yl)methyl]cyclohexyl]oxy}piperidin-1- yl)-2,3-dihydro-1H-isoindol-2- yl]piperidine-2,6-dione | Scheme A |
| 158 | 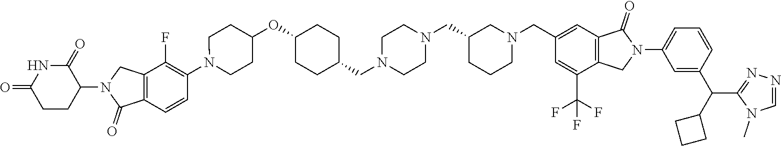 [SMILES: Cn1cnnc1C(c1cccc(N2Cc3c(cc(CN4CCC[C@H](CN5CCN(C[C@H]6CC[C@@H](OC7CCN(c8ccc9c(c8F)CN(C8CCC(=O)NC8=O)C9=O)CC7)CC6)CC5)C4)cc3C(F)(F)F)C2=O)c1)C1CCC1] | 3-[4-fluoro-1-oxo-5-(4-{[(1r,4r)-4-[(4- {[(3S)-1-[(2-{3-[cyclobutyl(4-methyl- 4H-1,2,4-triazol-3-yl)methyl]phenyl}- 3-oxo-7-(trifluoromethyl)-2,3-dihydro- 1H-isoindol-5-yl)methyl]piperidin-3- yl]methyl}piperazin-1- yl)methyl]cyclohexyl]oxy}piperidin-1- yl)-2,3-dihydro-1H-isoindol-2- yl]piperidine-2,6-dione | Scheme A |
| 159 | 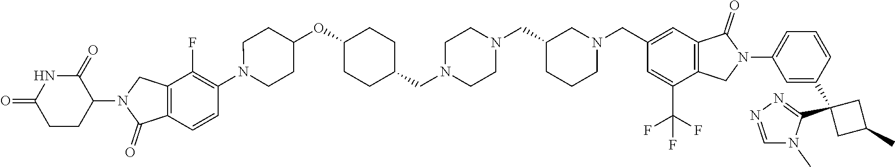 [SMILES: Cn1cnnc1[C@]1(c2cccc(N3Cc4c(cc(CN5CCC[C@H](CN6CCN(C[C@H]7CC[C@@H](OC8CCN(c9ccc%10c(c9F)CN(C9CCC(=O)NC9=O)C%10=O)CC8)CC7)CC6)C5)cc4C(F)(F)F)C3=O)c2)C[C@H](C)C1] | 3-[4-fluoro-1-oxo-5-(4-{[(1r,4r)-4-[(4- {[(3S)-1-[(3-oxo-2-{3-[(1s,3r)-3- methyl-1-(4-methyl-4H-1,2,4-triazol-3- yl)cyclobutyl]phenyl}-7- (trifluoromethyl)-2,3-dihydro-1H- isoindol-5-yl)methyl]piperidin-3- yl]methyl}piperazin-1- yl)methyl]cyclohexyl]oxy}piperidin-1- yl)-2,3-dihydro-1H-isoindol-2- yl]piperidine-2,6-dione | Scheme A |
| 160 | 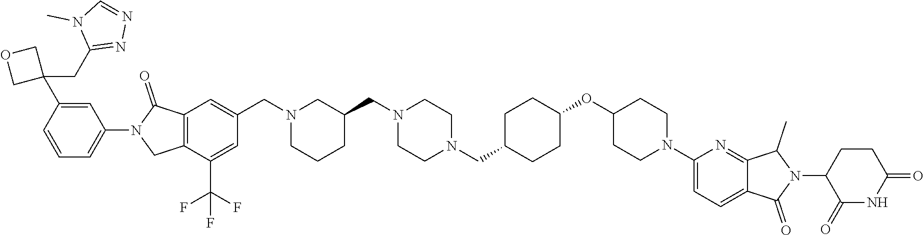 [SMILES: CC1c2nc(N3CCC(O[C@H]4CC[C@@H](CN5CCN(C[C@H]6CCCN(Cc7cc8c(c(C(F)(F)F)c7)CN(c7cccc(C9(Cc%10nncn%10C)COC9)c7)C8=O)C6)CC5)CC4)CC3)ccc2C(=O)N1C1CCC(=O)NC1=O] | 3-[7-methyl-5-oxo-2-(4-{[(1r,4r)-4-[(4- {[(3S)-1-{[2-(3-{3-[(4-methyl-4H- 1,2,4-triazol-3-yl)methyl]oxetan-3- yl}phenyl)-3-oxo-7-(trifluoromethyl)- 2,3-dihydro-1H-isoindol-5- yl]methyl}piperidin-3- yl]methyl}piperazin-1- yl)methyl]cyclohexyl]oxy}piperidin-1- yl)-5H,6H,7H-pyrrolo[3,4-b]pyridin-6- yl]piperidine-2,6-dione | Scheme A |
| 161 | 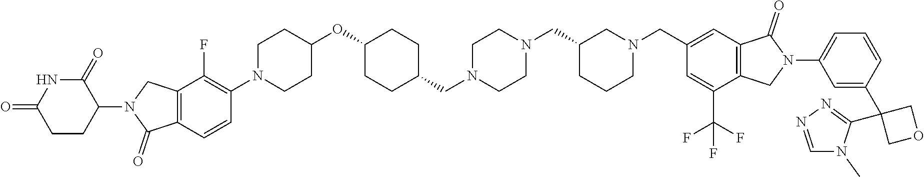 [SMILES: Cn1cnnc1C1(c2cccc(N3Cc4c(cc(CN5CCC[C@H](CN6CCN(C[C@H]7CC[C@@H](OC8CCN(c9ccc%10c(c9F)CN(C9CCC(=O)NC9=O)C%10=O)CC8)CC7)CC6)C5)cc4C(F)(F)F)C3=O)c2)COC1] | 3-[4-fluoro-1-oxo-5-(4-{[(1r,4r)-4-[(4- {[(3S)-1-[(2-{3-[3-(4-methyl-4H-1,2,4- triazol-3-yl)oxetan-3-yl]phenyl}-3-oxo- 7-(trifluoromethyl)-2,3-dihydro-1H- isoindol-5-yl)methyl]piperidin-3- yl]methyl}piperazin-1- yl)methyl]cyclohexyl]oxy}piperidin-1- yl)-2,3-dihydro-1H-isoindol-2- yl]piperidine-2,6-dione | Scheme A |
| 162 | 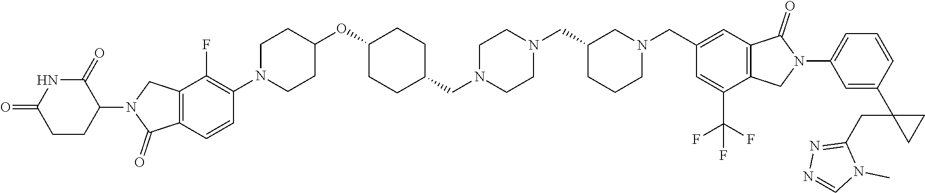 [SMILES: Cn1cnnc1CC1(c2cccc(N3Cc4c(cc(CN5CCC[C@H](CN6CCN(C[C@H]7CC[C@@H](OC8CCN(c9ccc%10c(c9F)CN(C9CCC(=O)NC9=O)C%10=O)CC8)CC7)CC6)C5)cc4C(F)(F)F)C3=O)c2)CC1] | 3-[4-fluoro-1-oxo-5-(4-{[(1r,4r)-4-[(4- {[(3S)-1-{[2-(3-{1-[(4-methyl-4H- 1,2,4-triazol-3- yl)methyl]cyclopropyl}phenyl)-3-oxo- 7-(trifluoromethyl)-2,3-dihydro-1H- isoindol-5-yl]methyl}piperidin-3- yl]methyl}piperazin-1- yl)methyl]cyclohexyl]oxy}piperidin-1- yl)-2,3-dihydro-1H-isoindol-2- yl]piperidine-2,6-dione | Scheme A |
| 163 | 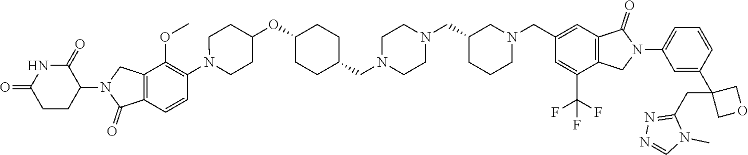 [SMILES: COc1c(N2CCC(O[C@H]3CC[C@@H](CN4CCN(C[C@H]5CCCN(Cc6cc7c(c(C(F)(F)F)c6)CN(c6cccc(C8(Cc9nncn9C)COC8)c6)C7=O)C5)CC4)CC3)CC2)ccc2c1CN(C1CCC(=O)NC1=O)C2=O] | 3-[4-methoxy-1-oxo-5-(4-{[(1r,4r)-4- [(4-{[(3S)-1-{[2-(3-{3-[(4-methyl-4H- 1,2,4-triazol-3-yl)methyl]oxetan-3- yl}phenyl)-3-oxo-7-(trifluoromethyl)- 2,3-dihydro-1H-isoindol-5- yl]methyl}piperidin-3- yl]methyl}piperazin-1- yl)methyl]cyclohexyl]oxy}piperidin-1- yl)-2,3-dihydro-1H-isoindol-2- yl]piperidine-2,6-dione | Scheme A |
| 164 | 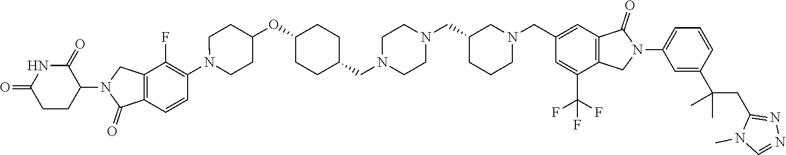 [SMILES: Cn1cnnc1CC(C)(C)c1cccc(N2Cc3c(cc(CN4CCC[C@H](CN5CCN(C[C@H]6CC[C@@H](OC7CCN(c8ccc9c(c8F)CN(C8CCC(=O)NC8=O)C9=O)CC7)CC6)CC5)C4)cc3C(F)(F)F)C2=O)c1] | 3-[4-fluoro-1-oxo-5-(4-{[(1r,4r)-4-[(4- {[(3S)-1-[(2-{3-[2-methyl-1-(4-methyl- 4H-1,2,4-triazol-3-yl)propan-2- yl]phenyl}-3-oxo-7-(trifluoromethyl)- 2,3-dihydro-1H-isoindol-5- yl)methyl]piperidin-3- yl]methyl}piperazin-1- yl)methyl]cyclohexyl]oxy}piperidin-1- yl)-2,3-dihydro-1H-isoindol-2- yl]piperidine-2,6-dione | Scheme A |
| 165 | 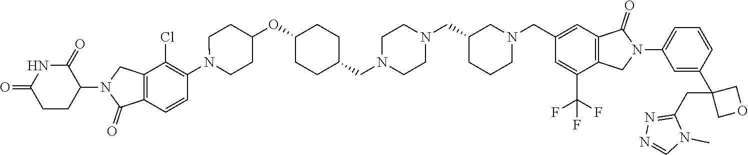 [SMILES: Cn1cnnc1CC1(c2cccc(N3Cc4c(cc(CN5CCC[C@H](CN6CCN(C[C@H]7CC[C@@H](OC8CCN(c9ccc%10c(c9Cl)CN(C9CCC(=O)NC9=O)C%10=O)CC8)CC7)CC6)C5)cc4C(F)(F)F)C3=O)c2)COC1] | 3-[4-chloro-1-oxo-5-(4-{[(1r,4r)-4-[(4- {[(3S)-1-{[2-(3-{3-[(4-methyl-4H- 1,2,4-triazol-3-yl)methyl]oxetan-3- yl}phenyl)-3-oxo-7-(trifluoromethyl)- 2,3-dihydro-1H-isoindol-5- yl]methyl}piperidin-3- yl]methyl}piperazin-1- yl)methyl]cyclohexyl]oxy}piperidin-1- yl)-2,3-dihydro-1H-isoindol-2- yl]piperidine-2,6-dione | Scheme A |
| 166 | 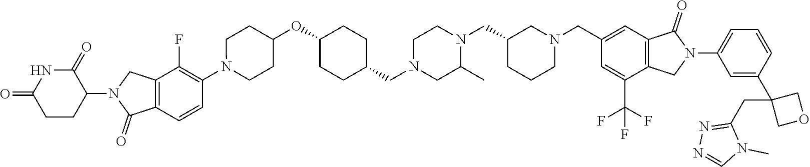 [SMILES: CC1CN(C[C@H]2CC[C@@H](OC3CCN(c4ccc5c(c4F)CN(C4CCC(=O)NC4=O)C5=O)CC3)CC2)CCN1C[C@H]1CCCN(Cc2cc3c(c(C(F)(F)F)c2)CN(c2cccc(C4(Cc5nncn5C)COC4)c2)C3=O)C1] | 3-[4-fluoro-1-oxo-5-(4-{[(1r,4r)-4-[(3- methyl-4-{[(3S)-1-{[2-(3-{3-[(4- methyl-4H-1,2,4-triazol-3- yl)methyl]oxetan-3-yl}phenyl)-3-oxo- 7-(trifluoromethyl)-2,3-dihydro-1H- isoindol-5-yl]methyl}piperidin-3- yl]methyl}piperazin-1- yl)methyl]cyclohexyl]oxy}piperidin-1- yl)-2,3-dihydro-1H-isoindol-2- yl]piperidine-2,6-dione | Scheme A |
| 167 | 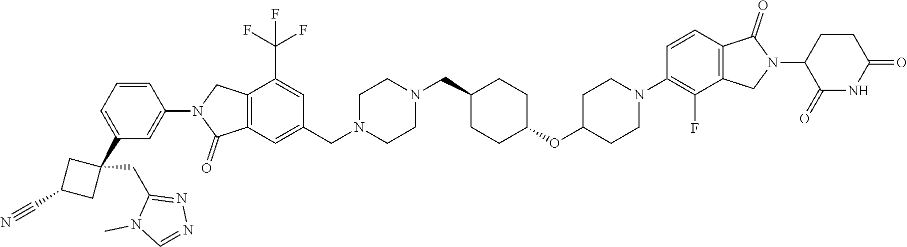 [SMILES: Cn1cnnc1C[C@]1(c2cccc(N3Cc4c(cc(CN5CCN(C[C@H]6CC[C@H](OC7CCN(c8ccc9c(c8F)CN(C8CCC(=O)NC8=O)C9=O)CC7)CC6)CC5)cc4C(F)(F)F)C3=O)c2)C[C@H](C#N)C1] | (1r,3r)-3-[(4-methyl-4H-1,2,4-triazol-3- yl)methyl]-3-(3-{1-oxo-6-[(4-{[(1r,4r)- 4-({1-[2-(2,6-dioxopiperidin-3-yl)-4- fluoro-1-oxo-2,3-dihydro-1H-isoindol- 5-yl]piperidin-4- yl}oxy)cyclohexyl]methyl}piperazin-1- yl)methyl]-4-(trifluoromethyl)-2,3- dihydro-1H-isoindol-2- yl}phenyl)cyclobutane-1-carbonitrile | Scheme A |
| 168 | 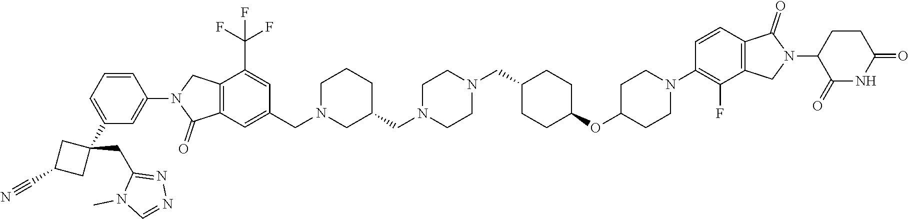 [SMILES: Cn1cnnc1C[C@]1(c2cccc(N3Cc4c(cc(CN5CCC[C@H](CN6CCN(C[C@H]7CC[C@H](OC8CCN(c9ccc%10c(c9F)CN(C9CCC(=O)NC9=O)C%10=O)CC8)CC7)CC6)C5)cc4C(F)(F)F)C3=O)c2)C[C@@H](C#N)C1] | (1r,3r)-3-[(4-methyl-4H-1,2,4-triazol-3- yl)methyl]-3-[3-(1-oxo-6-{[(3S)-3-[(4- {[(1r,4r)-4-({1-[2-(2,6-dioxopiperidin- 3-yl)-4-fluoro-1-oxo-2,3-dihydro-1H- isoindol-5-yl]piperidin-4- yl}oxy)cyclohexyl]methyl}piperazin-1- yl)methyl]piperidin-1-yl]methyl}-4- (trifluoromethyl)-2,3-dihydro-1H- isoindol-2-yl)phenyl]cyclobutane-1- carbonitrile | Scheme A |
| 169 | 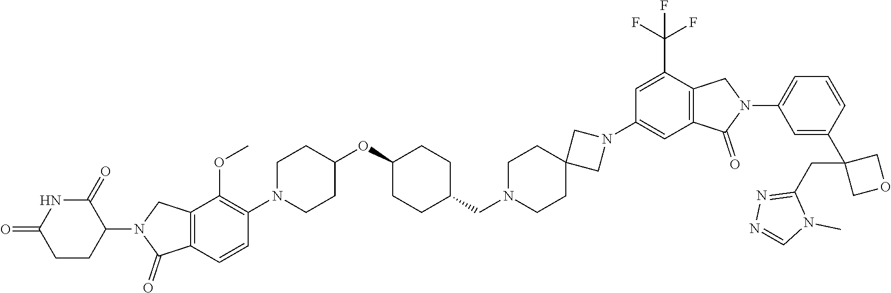 [SMILES: COc1c(N2CCC(O[C@H]3CC[C@H](CN4CCC5(CC4)CN(c4cc6c(c(C(F)(F)F)c4)CN(c4cccc(C7(Cc8nncn8C)COC7)c4)C6=O)C5)CC3)CC2)ccc2c1CN(C1CCC(=O)NC1=O)C2=O] | 3-[4-methoxy-1-oxo-5-(4-{[(1r,4r)-4- ({2-[2-(3-{3-[(4-methyl-4H-1,2,4- triazol-3-yl)methyl]oxetan-3- yl}phenyl)-3-oxo-7-(trifluoromethyl)- 2,3-dihydro-1H-isoindol-5-yl]-2,7- diazaspiro[3.5]nonan-7- yl}methyl)cyclohexyl]oxy}piperidin-1- yl)-2,3-dihydro-1H-isoindol-2- yl]piperidine-2,6-dione | Scheme B |
| 170 | 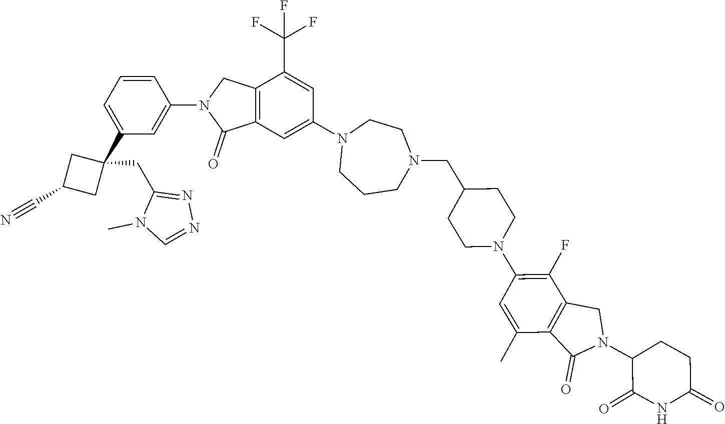 [SMILES: Cc1cc(N2CCC(CN3CCCN(c4cc5c(c(C(F)(F)F)c4)CN(c4cccc([C@]6(Cc7nncn7C)C[C@@H](C#N)C6)c4)C5=O)CC3)CC2)c(F)c2c1C(=O)N(C1CCC(=O)NC1=O)C2] | (1r,3r)-3-(3-{6-[4-({1-[2-(2,6- dioxopiperidin-3-yl)-4-fluoro-7- methyl-1-oxo-2,3-dihydro-1H-isoindol- 5-yl]piperidin-4-yl}methyl)-1,4- diazepan-1-yl]-1-oxo-4- (trifluoromethyl)-2,3-dihydro-1H- isoindol-2-yl}phenyl)-3-[(4-methyl- 4H-1,2,4-triazol-3- yl)methyl]cyclobutane-1-carbonitrile | Scheme B |
| 171 | 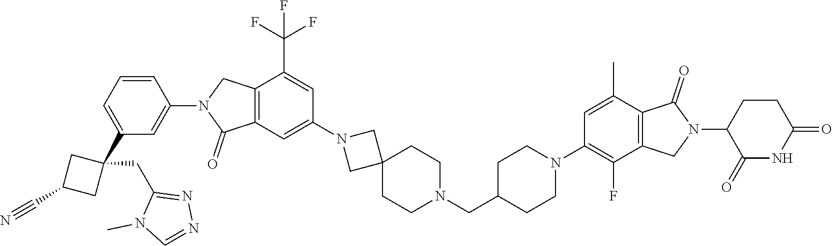 [SMILES: Cc1cc(N2CCC(CN3CCC4(CC3)CN(c3cc5c(c(C(F)(F)F)c3)CN(c3cccc([C@]6(Cc7nncn7C)C[C@@H](C#N)C6)c3)C5=O)C4)CC2)c(F)c2c1C(=O)N(C1CCC(=O)NC1=O)C2] | (1r,3r)-3-(3-{6-[7-({1-[2-(2,6- dioxopiperidin-3-yl)-4-fluoro-7- methyl-1-oxo-2,3-dihydro-1H-isoindol- 5-yl]piperidin-4-yl}methyl)-2,7- diazaspiro[3.5]nonan-2-yl]-1-oxo-4- (trifluoromethyl)-2,3-dihydro-1H- isoindol-2-yl}phenyl)-3-[(4-methyl- 4H-1,2,4-triazol-3- yl)methyl]cyclobutane-1-carbonitrile | Scheme B |
| 172 | 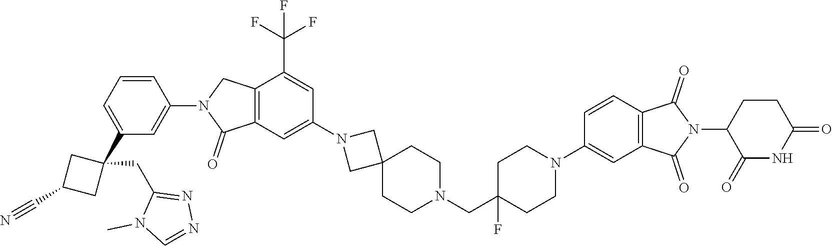 [SMILES: Cn1cnnc1C[C@]1(c2cccc(N3Cc4c(cc(N5CC6(CCN(CC7(F)CCN(c8ccc9c(c8)C(=O)N(C8CCC(=O)NC8=O)C9=O)CC7)CC6)C5)cc4C(F)(F)F)C3=O)c2)C[C@H](C#N)C1] | (1r,3r)-3-(3-{6-[7-({1-[2-(2,6- dioxopiperidin-3-yl)-1,3-dioxo-2,3- dihydro-1H-isoindol-5-yl]-4- fluoropiperidin-4-yl}methyl)-2,7- diazaspiro[3.5]nonan-2-yl]-1-oxo-4- (trifluoromethyl)-2,3-dihydro-1H- isoindol-2-yl}phenyl)-3-[(4-methyl- 4H-1,2,4-triazol-3- yl)methyl]cyclobutane-1-carbonitrile | Scheme B |
| 173 | 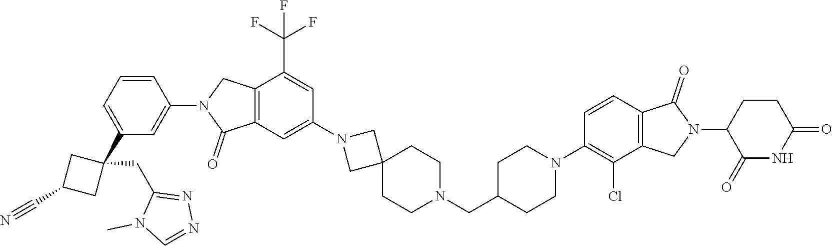 [SMILES: Cn1cnnc1C[C@]1(c2cccc(N3Cc4c(cc(N5CC6(CCN(CC7CCN(c8ccc9c(c8Cl)CN(C8CCC(=O)NC8=O)C9=O)CC7)CC6)C5)cc4C(F)(F)F)C3=O)c2)C[C@H](C#N)C1] | (1r,3r)-3-(3-{6-[7-({1-[4-chloro-2-(2,6- dioxopiperidin-3-yl)-1-oxo-2,3- dihydro-1H-isoindol-5-yl]piperidin-4- yl}methyl)-2,7-diazaspiro[3.5]nonan-2- yl]-1-oxo-4-(trifluoromethyl)-2,3- dihydro-1H-isoindol-2-yl}phenyl)-3- [(4-methyl-4H-1,2,4-triazol-3- yl)methyl]cyclobutane-1-carbonitrile | Scheme B |
| 174 | 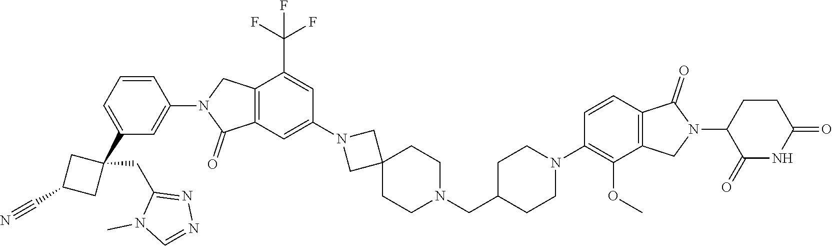 [SMILES: COc1c(N2CCC(CN3CCC4(CC3)CN(c3cc5c(c(C(F)(F)F)c3)CN(c3cccc([C@]6(Cc7nncn7C)C[C@@H](C#N)C6)c3)C5=O)C4)CC2)ccc2c1CN(C1CCC(=O)NC1=O)C2=O] | (1r,3r)-3-(3-{6-[7-({1-[2-(2,6- dioxopiperidin-3-yl)-4-methoxy-1-oxo- 2,3-dihydro-1H-isoindol-5- yl]piperidin-4-yl}methyl)-2,7- diazaspiro[3.5]nonan-2-yl]-1-oxo-4- (trifluoromethyl)-2,3-dihydro-1H- isoindol-2-yl}phenyl)-3-[(4-methyl- 4H-1,2,4-triazol-3- yl)methyl]cyclobutane-1-carbonitrile | Scheme B |
| 175 | 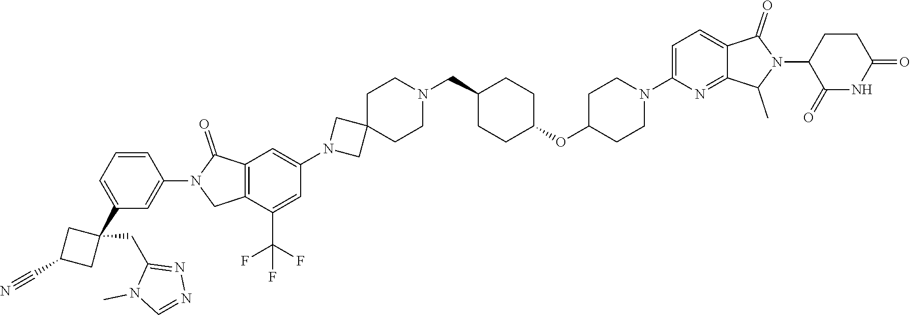 [SMILES: CC1c2nc(N3CCC(O[C@H]4CC[C@H](CN5CCC6(CC5)CN(c5cc7c(c(C(F)(F)F)c5)CN(c5cccc([C@]8(Cc9nncn9C)C[C@@H](C#N)C8)c5)C7=O)C6)CC4)CC3)ccc2C(=O)N1C1CCC(=O)NC1=O] | (1r,3r)-3-[(4-methyl-4H-1,2,4-triazol-3- yl)methyl]-3-{3-[1-oxo-6-(7-{[(1r,4r)- 4-({1-[6-(2,6-dioxopiperidin-3-yl)-7- methyl-5-oxo-5H,6H,7H-pyrrolo[3,4- b]pyridin-2-yl]piperidin-4- yl}oxy)cyclohexyl]methyl}-2,7- diazaspiro[3.5]nonan-2-yl)-4- (trifluoromethyl)-2,3-dihydro-1H- isoindol-2-yl]phenyl}cyclobutane-1- carbonitrile | Scheme B |
| 176 | 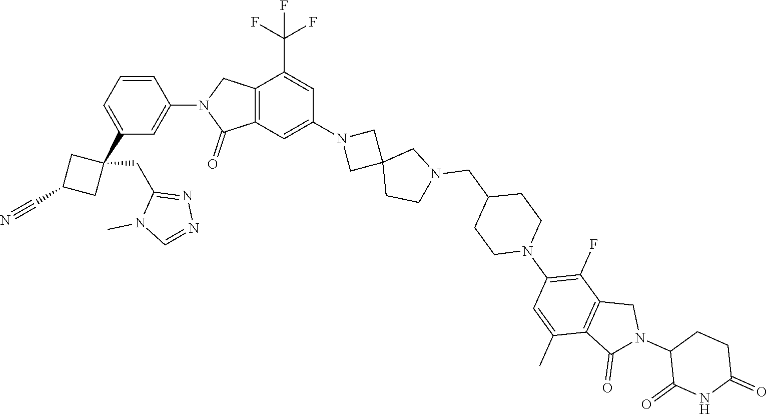 [SMILES: Cc1cc(N2CCC(CN3CCC4(C3)CN(c3cc5c(c(C(F)(F)F)c3)CN(c3cccc([C@]6(Cc7nncn7C)C[C@@H](C#N)C6)c3)C5=O)C4)CC2)c(F)c2c1C(=O)N(C1CCC(=O)NC1=O)C2] | (1r,3r)-3-(3-{6-[6-({1-[2-(2,6- dioxopiperidin-3-yl)-4-fluoro-7- methyl-1-oxo-2,3-dihydro-1H-isoindol- 5-yl]piperidin-4-yl}methyl)-2,6- diazaspiro[3.4]octan-2-yl]-1-oxo-4- (trifluoromethyl)-2,3-dihydro-1H- isoindol-2-yl}phenyl)-3-[(4-methyl- 4H-1,2,4-triazol-3- yl)methyl]cyclobutane-1-carbonitrile | Scheme B |
| 177 | 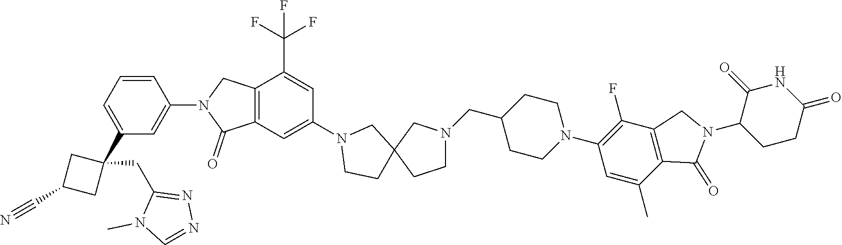 [SMILES: Cc1cc(N2CCC(CN3CCC4(CCN(c5cc6c(c(C(F)(F)F)c5)CN(c5cccc([C@]7(Cc8nncn8C)C[C@@H](C#N)C7)c5)C6=O)C4)C3)CC2)c(F)c2c1C(=O)N(C1CCC(=O)NC1=O)C2] | (1r,3r)-3-(3-{6-[7-({1-[2-(2,6- dioxopiperidin-3-yl)-4-fluoro-7- methyl-1-oxo-2,3-dihydro-1H-isoindol- 5-yl]piperidin-4-yl}methyl)-2,7- diazaspiro[4.4]nonan-2-yl]-1-oxo-4- (trifluoromethyl)-2,3-dihydro-1H- isoindol-2-yl}phenyl)-3-[(4-methyl- 4H-1,2,4-triazol-3- yl)methyl]cyclobutane-1-carbonitrile | Scheme B |
| 178 | 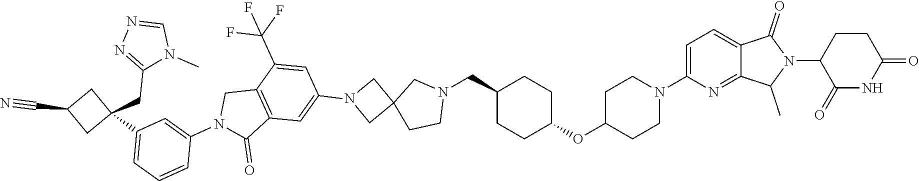 [SMILES: CC1c2nc(N3CCC(O[C@H]4CC[C@H](CN5CCC6(C5)CN(c5cc7c(c(C(F)(F)F)c5)CN(c5cccc([C@]8(Cc9nncn9C)C[C@@H](C#N)C8)c5)C7=O)C6)CC4)CC3)ccc2C(=O)N1C1CCC(=O)NC1=O] | (1r,3r)-3-[(4-methyl-4H-1,2,4-triazol-3- yl)methyl]-3-{3-[1-oxo-6-(6-{[(1r,4r)- 4-({1-[6-(2,6-dioxopiperidin-3-yl)-7- methyl-5-oxo-5H,6H,7H-pyrrolo[3,4- b]pyridin-2-yl]piperidin-4- yl}oxy)cyclohexyl]methyl}-2,6- diazaspiro[3.4]octan-2-yl)-4- (trifluoromethyl)-2,3-dihydro-1H- isoindol-2-yl]phenyl}cyclobutane-1- carbonitrile | Scheme B |
| 179 | 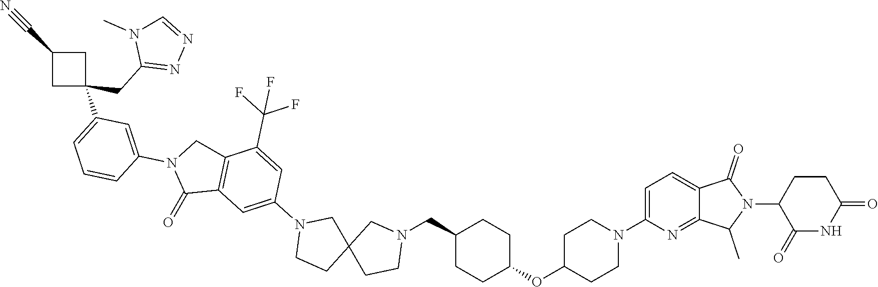 [SMILES: CC1c2nc(N3CCC(O[C@H]4CC[C@H](CN5CCC6(CCN(c7cc8c(c(C(F)(F)F)c7)CN(c7cccc([C@]9(Cc%10nncn%10C)C[C@@H](C#N)C9)c7)C8=O)C6)C5)CC4)CC3)ccc2C(=O)N1C1CCC(=O)NC1=O] | (1r,3r)-3-[(4-methyl-4H-1,2,4-triazol-3- yl)methyl]-3-{3-[1-oxo-6-(7-{[(1r,4r)- 4-({1-[6-(2,6-dioxopiperidin-3-yl)-7- methyl-5-oxo-5H,6H,7H-pyrrolo[3,4- b]pyridin-2-yl]piperidin-4- yl}oxy)cyclohexyl]methyl}-2,7- diazaspiro[4.4]nonan-2-yl)-4- (trifluoromethyl)-2,3-dihydro-1H- isoindol-2-yl]phenyl}cyclobutane-1- carbonitrile | Scheme B |
| 180 | 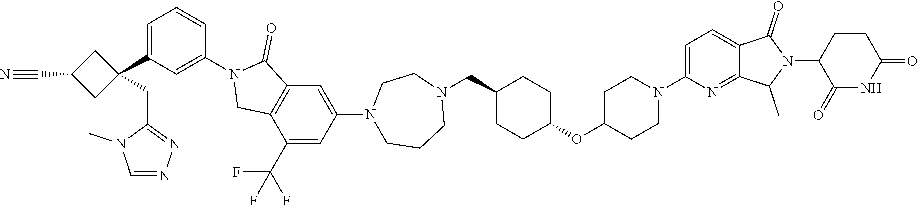 [SMILES: CC1c2nc(N3CCC(O[C@H]4CC[C@H](CN5CCCN(c6cc7c(c(C(F)(F)F)c6)CN(c6cccc([C@]8(Cc9nncn9C)C[C@@H](C#N)C8)c6)C7=O)CC5)CC4)CC3)ccc2C(=O)N1C1CCC(=O)NC1=O] | (1r,3r)-3-[(4-methyl-4H-1,2,4-triazol-3- yl)methyl]-3-{3-[1-oxo-6-(4-{[(1r,4r)- 4-({1-[6-(2,6-dioxopiperidin-3-yl)-7- methyl-5-oxo-5H,6H,7H-pyrrolo[3,4- b]pyridin-2-yl]piperidin-4- yl}oxy)cyclohexyl]methyl}-1,4- diazepan-1-yl)-4-(trifluoromethyl)-2,3- dihydro-1H-isoindol-2- yl]phenyl}cyclobutane-1-carbonitrile | Scheme B |
| 181 | 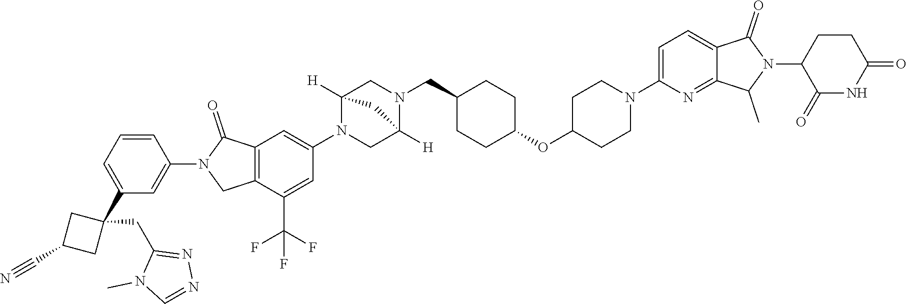 [SMILES: CC1c2nc(N3CCC(O[C@H]4CC[C@H](CN5C[C@H]6C[C@@H]5CN6c5cc6c(c(C(F)(F)F)c5)CN(c5cccc([C@]7(Cc8nncn8C)C[C@@H](C#N)C7)c5)C6=O)CC4)CC3)ccc2C(=O)N1C1CCC(=O)NC1=O] | (1r,3r)-3-[(4-methyl-4H-1,2,4-triazol-3- yl)methyl]-3-(3-{1-oxo-6-[(1R,4R)-5- {[(1r,4r)-4-({1-[6-(2,6-dioxopiperidin- 3-yl)-7-methyl-5-oxo-5H,6H,7H- pyrrolo[3,4-b]pyridin-2-yl]piperidin-4- yl}oxy)cyclohexyl]methyl}-2,5- diazabicyclo[2.2.1]heptan-2-yl]-4- (trifluoromethyl)-2,3-dihydro-1H- isoindol-2-yl}phenyl)cyclobutane-1- carbonitrile | Scheme B |
| 182 | 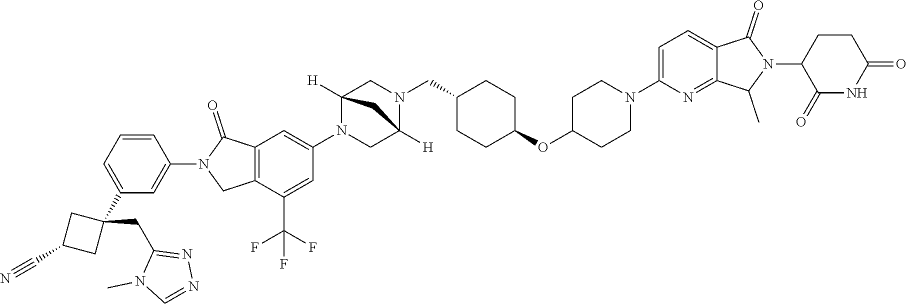 [SMILES: CC1c2nc(N3CCC(O[C@H]4CC[C@H](CN5C[C@@H]6C[C@H]5CN6c5cc6c(c(C(F)(F)F)c5)CN(c5cccc([C@]7(Cc8nncn8C)C[C@H](C#N)C7)c5)C6=O)CC4)CC3)ccc2C(=O)N1C1CCC(=O)NC1=O] | (1r,3r)-3-[(4-methyl-4H-1,2,4-triazol-3- yl)methyl]-3-(3-{1-oxo-6-[(1S,4S)-5- {[(1r,4r)-4-({1-[6-(2,6-dioxopiperidin- 3-yl)-7-methyl-5-oxo-5H,6H,7H- pyrrolo[3,4-b]pyridin-2-yl]piperidin-4- yl}oxy)cyclohexyl]methyl}-2,5- diazabicyclo[2.2.1]heptan-2-yl]-4- (trifluoromethyl)-2,3-dihydro-1H- isoindol-2-yl}phenyl)cyclobutane-1- carbonitrile | Scheme B |
| 183 | 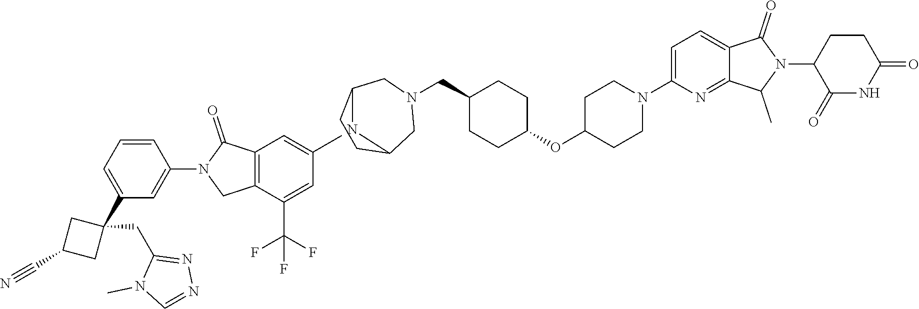 | (1r,3r)-3-[(4-methyl-4H-1,2,4-triazol-3- yl)methyl]-3-{3-[1-oxo-6-(3-{[(1r,4r)- 4-({1-[6-(2,6-dioxopiperidin-3-yl)-7- methyl-5-oxo-5H,6H,7H-pyrrolo[3,4- b]pyridin-2-yl]piperidin-4- yl}oxy)cyclohexyl]methyl}-3,8- diazabicyclo[3.2.1]octan-8-yl)-4- (trifluoromethyl)-2,3-dihydro-1H- isoindol-2-yl]phenyl}cyclobutane-1- carbonitrile | Scheme B |
| 184 | 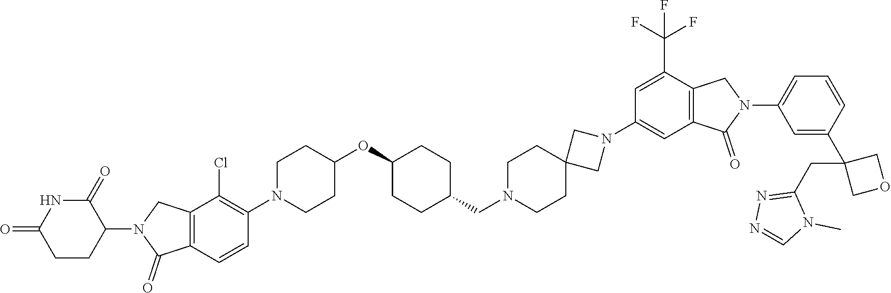 [SMILES: Cn1cnnc1CC1(c2cccc(N3Cc4c(cc(N5CC6(CCN(C[C@H]7CC[C@H](OC8CCN(c9ccc%10c(c9Cl)CN(C9CCC(=O)NC9=O)C%10=O)CC8)CC7)CC6)C5)cc4C(F)(F)F)C3=O)c2)COC1] | 3-[4-chloro-1-oxo-5-(4-{[(1r,4r)-4-({2- [2-(3-{3-[(4-methyl-4H-1,2,4-triazol-3- yl)methyl]oxetan-3-yl}phenyl)-3-oxo- 7-(trifluoromethyl)-2,3-dihydro-1H- isoindol-5-yl]-2,7- diazaspiro[3.5]nonan-7- yl}methyl)cyclohexyl]oxy}piperidin-1- yl)-2,3-dihydro-1H-isoindol-2- yl]piperidine-2,6-dione | Scheme B |
| 185 | 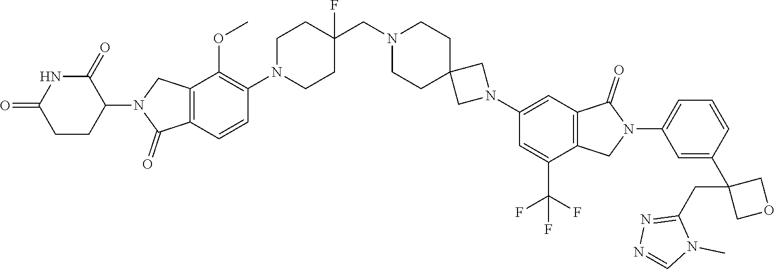 [SMILES: COc1c(N2CCC(F)(CN3CCC4(CC3)CN(c3cc5c(c(C(F)(F)F)c3)CN(c3cccc(C6(Cc7nncn7C)COC6)c3)C5=O)C4)CC2)ccc2c1CN(C1CCC(=O)NC1=O)C2=O] | 3-{5-[4-fluoro-4-({2-[2-(3-{3-[(4- methyl-4H-1,2,4-triazol-3- yl)methyl]oxetan-3-yl}phenyl)-3-oxo- 7-(trifluoromethyl)-2,3-dihydro-1H- isoindol-5-yl]-2,7- diazaspiro[3.5]nonan-7- yl}methyl)piperidin-1-yl]-4-methoxy- 1-oxo-2,3-dihydro-1H-isoindol-2- yl}piperidine-2,6-dione | Scheme B |
| 186 | 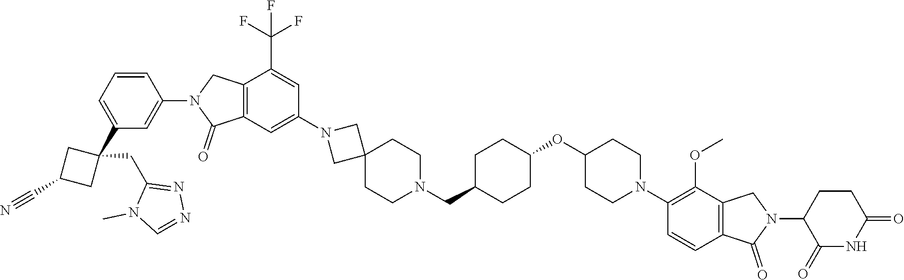 [SMILES: COc1c(N2CCC(O[C@H]3CC[C@H](CN4CCC5(CC4)CN(c4cc6c(c(C(F)(F)F)c4)CN(c4cccc([C@]7(Cc8nncn8C)C[C@@H](C#N)C7)c4)C6=O)C5)CC3)CC2)ccc2c1CN(C1CCC(=O)NC1=O)C2=O] | (1r,3r)-3-[(4-methyl-4H-1,2,4-triazol-3- yl)methyl]-3-{3-[1-oxo-6-(7-{[(1r,4r)- 4-({1-[2-(2,6-dioxopiperidin-3-yl)-4- methoxy-1-oxo-2,3-dihydro-1H- isoindol-5-yl]piperidin-4- yl}oxy)cyclohexyl]methyl}-2,7- diazaspiro[3.5]nonan-2-yl)-4- (trifluoromethyl)-2,3-dihydro-1H- isoindol-2-yl]phenyl}cyclobutane-1- carbonitrile | Scheme B |
| 187 | 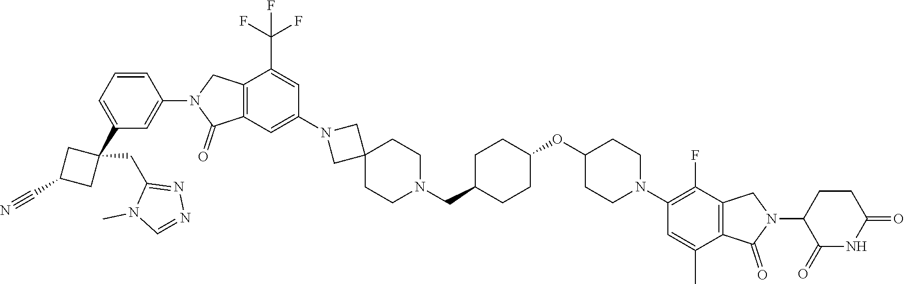 [SMILES: Cc1cc(N2CCC(O[C@H]3CC[C@H](CN4CCC5(CC4)CN(c4cc6c(c(C(F)(F)F)c4)CN(c4cccc([C@]7(Cc8nncn8C)C[C@@H](C#N)C7)c4)C6=O)C5)CC3)CC2)c(F)c2c1C(=O)N(C1CCC(=O)NC1=O)C2] | (1r,3r)-3-[(4-methyl-4H-1,2,4-triazol-3- yl)methyl]-3-{3-[1-oxo-6-(7-{[(1r,4r)- 4-({1-[2-(2,6-dioxopiperidin-3-yl)-4- fluoro-7-methyl-1-oxo-2,3-dihydro-1H- isoindol-5-yl]piperidin-4- yl}oxy)cyclohexyl]methyl}-2,7- diazaspiro[3.5]nonan-2-yl)-4- (trifluoromethyl)-2,3-dihydro-1H- isoindol-2-yl]phenyl}cyclobutane-1- carbonitrile | Scheme B |
| 188 | 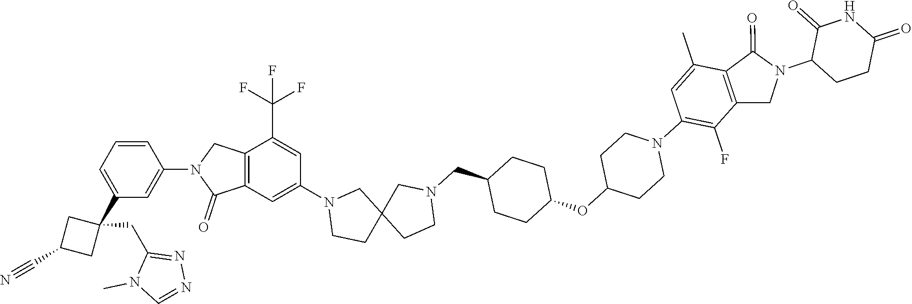 [SMILES: Cc1cc(N2CCC(O[C@H]3CC[C@H](CN4CCC5(CCN(c6cc7c(c(C(F)(F)F)c6)CN(c6cccc([C@]8(Cc9nncn9C)C[C@@H](C#N)C8)c6)C7=O)C5)C4)CC3)CC2)c(F)c2c1C(=O)N(C1CCC(=O)NC1=O)C2] | (1r,3r)-3-[(4-methyl-4H-1,2,4-triazol-3- yl)methyl]-3-{3-[1-oxo-6-(7-{[(1r,4r)- 4-({1-[2-(2,6-dioxopiperidin-3-yl)-4- fluoro-7-methyl-1-oxo-2,3-dihydro-1H- isoindol-5-yl]piperidin-4- yl}oxy)cyclohexyl]methyl}-2,7- diazaspiro[4.4]nonan-2-yl)-4- (trifluoromethyl)-2,3-dihydro-1H- isoindol-2-yl]phenyl}cyclobutane-1- carbonitrile | Scheme B |
| 189 | 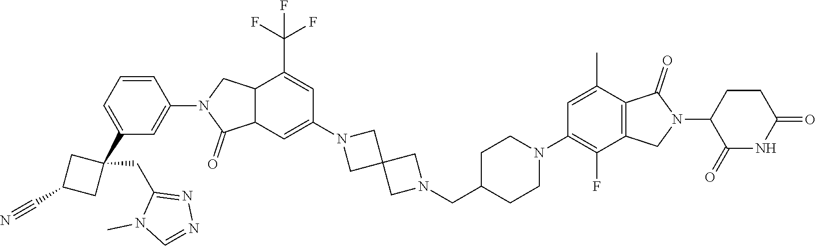 [SMILES: Cc1cc(N2CCC(CN3CC4(C3)CN(C3=CC5C(=O)N(c6cccc([C@]7(Cc8nncn8C)C[C@@H](C#N)C7)c6)CC5C(C(F)(F)F)=C3)C4)CC2)c(F)c2c1C(=O)N(C1CCC(=O)NC1=O)C2] | (1r,3r)-3-(3-{6-[6-({1-[2-(2,6- dioxopiperidin-3-yl)-4-fluoro-7- methyl-1-oxo-2,3-dihydro-1H-isoindol- 5-yl]piperidin-4-yl}methyl)-2,6- diazaspiro[3.3]heptan-2-yl]-1-oxo-4- (trifluoromethyl)-2,3,3a,7a-tetrahydro- 1H-isoindol-2-yl}phenyl)-3-[(4- methyl-4H-1,2,4-triazol-3- yl)methyl]cyclobutane-1-carbonitrile | Scheme B |
| 190 | 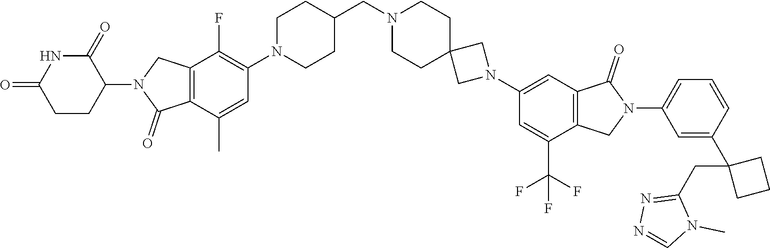 [SMILES: Cc1cc(N2CCC(CN3CCC4(CC3)CN(c3cc5c(c(C(F)(F)F)c3)CN(c3cccc(C6(Cc7nncn7C)CCC6)c3)C5=O)C4)CC2)c(F)c2c1C(=O)N(C1CCC(=O)NC1=O)C2] | 3-{4-fluoro-7-methyl-5-[4-({2-[2-(3- {1-[(4-methyl-4H-1,2,4-triazol-3- yl)methyl]cyclobutyl}phenyl)-3-oxo-7- (trifluoromethyl)-2,3-dihydro-1H- isoindol-5-yl]-2,7- diazaspiro[3.5]nonan-7- yl}methyl)piperidin-1-yl]-1-oxo-2,3- dihydro-1H-isoindol-2-yl}piperidine- 2,6-dione | Scheme B |
| 191 | 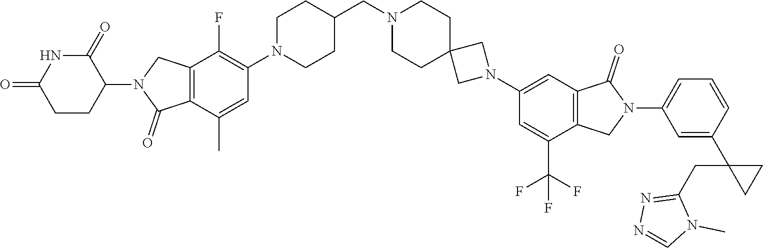 [SMILES: Cc1cc(N2CCC(CN3CCC4(CC3)CN(c3cc5c(c(C(F)(F)F)c3)CN(c3cccc(C6(Cc7nncn7C)CC6)c3)C5=O)C4)CC2)c(F)c2c1C(=O)N(C1CCC(=O)NC1=O)C2] | 3-{4-fluoro-7-methyl-5-[4-({2-[2-(3- {1-[(4-methyl-4H-1,2,4-triazol-3- yl)methyl]cyclopropyl}phenyl)-3-oxo- 7-(trifluoromethyl)-2,3-dihydro-1H- isoindol-5-yl]-2,7- diazaspiro[3.5]nonan-7- yl}methyl)piperidin-1-yl]-1-oxo-2,3- dihydro-1H-isoindol-2-yl}piperidine- 2,6-dione | Scheme B |
| 192 | 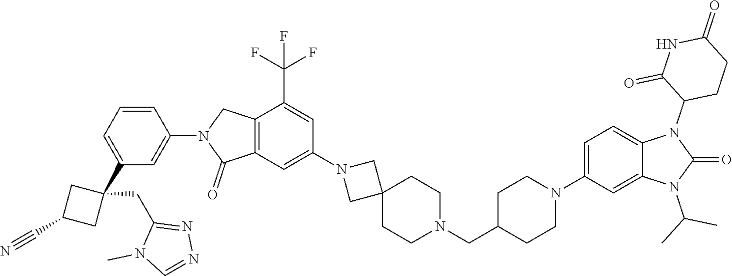 [SMILES: CC(C)n1c(=O)n(C2CCC(=O)NC2=O)c2ccc(N3CCC(CN4CCC5(CC4)CN(c4cc6c(c(C(F)(F)F)c4)CN(c4cccc([C@]7(Cc8nncn8C)C[C@@H](C#N)C7)c4)C6=O)C5)CC3)cc21] | (1r,3r)-3-(3-{6-[7-({1-[1-(2,6- dioxopiperidin-3-yl)-2-oxo-3-(propan- 2-yl)-2,3-dihydro-1H-1,3-benzodiazol- 5-yl]piperidin-4-yl}methyl)-2,7- diazaspiro[3.5]nonan-2-yl]-1-oxo-4- (trifluoromethyl)-2,3-dihydro-1H- isoindol-2-yl}phenyl)-3-[(4-methyl- 4H-1,2,4-triazol-3- yl)methyl]cyclobutane-1-carbonitrile | Scheme B |
| 193 | 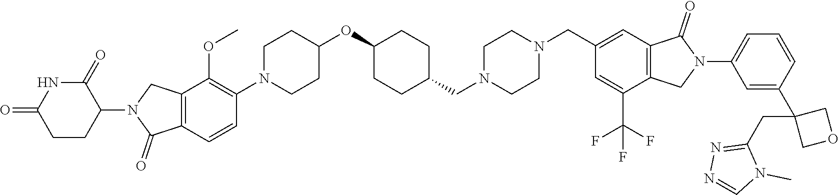 [SMILES: COc1c(N2CCC(O[C@H]3CC[C@H](CN4CCN(Cc5cc6c(c(C(F)(F)F)c5)CN(c5cccc(C7(Cc8nncn8C)COC7)c5)C6=O)CC4)CC3)CC2)ccc2c1CN(C1CCC(=O)NC1=O)C2=O] | 3-[4-methoxy-1-oxo-5-(4-{[(1r,4r)-4- [(4-{[2-(3-{3-[(4-methyl-4H-1,2,4- triazol-3-yl)methyl]oxetan-3- yl}phenyl)-3-oxo-7-(trifluoromethyl)- 2,3-dihydro-1H-isoindol-5- yl]methyl}piperazin-1- yl)methyl]cyclohexyl]oxy}piperidin-1- yl)-2,3-dihydro-1H-isoindol-2- yl]piperidine-2,6-dione | Scheme D |
| 194 | 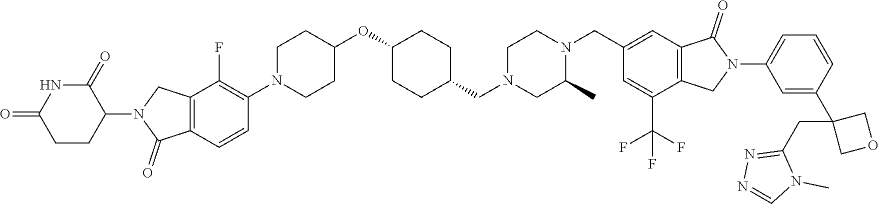 [SMILES: C[C@H]1CN(C[C@H]2CC[C@@H](OC3CCN(c4ccc5c(c4F)CN(C4CCC(=O)NC4=O)C5=O)CC3)CC2)CCN1Cc1cc2c(c(C(F)(F)F)c1)CN(c1cccc(C3(Cc4nncn4C)COC3)c1)C2=O] | 3-[4-fluoro-1-oxo-5-(4-{[(1r,4r)-4- {[(3S)-3-methyl-4-{[2-(3-{3-[(4- methyl-4H-1,2,4-triazol-3- yl)methyl]oxetan-3-yl}phenyl)-3-oxo- 7-(trifluoromethyl)-2,3-dihydro-1H- isoindol-5-yl]methyl}piperazin-1- yl]methyl}cyclohexyl]oxy}piperidin-1- yl)-2,3-dihydro-1H-isoindol-2- yl]piperidine-2,6-dione | Scheme D |
| 195 | 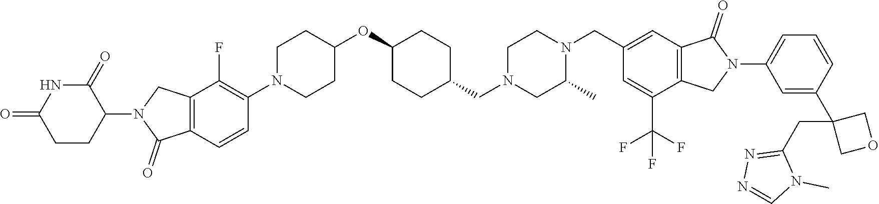 [SMILES: C[C@@H]1CN(C[C@H]2CC[C@H](OC3CCN(c4ccc5c(c4F)CN(C4CCC(=O)NC4=O)C5=O)CC3)CC2)CCN1Cc1cc2c(c(C(F)(F)F)c1)CN(c1cccc(C3(Cc4nncn4C)COC3)c1)C2=O] | 3-[4-fluoro-1-oxo-5-(4-{[(1r,4r)-4- {[(3R)-3-methyl-4-{[2-(3-{3-[(4- methyl-4H-1,2,4-triazol-3- yl)methyl]oxetan-3-yl}phenyl)-3-oxo- 7-(trifluoromethyl)-2,3-dihydro-1H- isoindol-5-yl]methyl}piperazin-1- yl]methyl}cyclohexyl]oxy}piperidin-1- yl)-2,3-dihydro-1H-isoindol-2- yl]piperidine-2,6-dione | Scheme D |
| 196 | 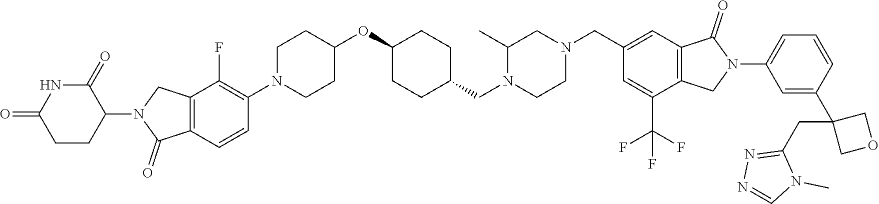 [SMILES: CC1CN(Cc2cc3c(c(C(F)(F)F)c2)CN(c2cccc(C4(Cc5nncn5C)COC4)c2)C3=O)CCN1C[C@H]1CC[C@H](OC2CCN(c3ccc4c(c3F)CN(C3CCC(=O)NC3=O)C4=O)CC2)CC1] | 3-[4-fluoro-1-oxo-5-(4-{[(1r,4r)-4-[(2- methyl-4-{[2-(3-{3-[(4-methyl-4H- 1,2,4-triazol-3-yl)methyl]oxetan-3- yl}phenyl)-3-oxo-7-(trifluoromethyl)- 2,3-dihydro-1H-isoindol-5- yl]methyl}piperazin-1- yl)methyl]cyclohexyl]oxy}piperidin-1- yl)-2,3-dihydro-1H-isoindol-2- yl]piperidine-2,6-dione | Scheme D |
| 197 | 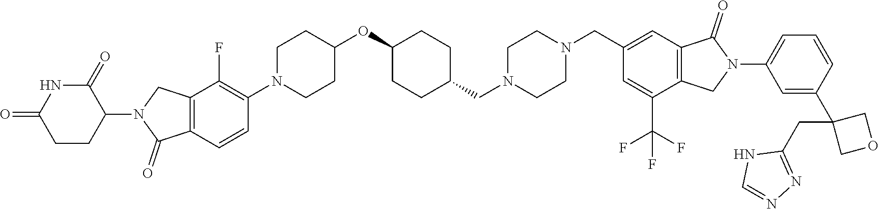 [SMILES: O=C1CCC(N2Cc3c(ccc(N4CCC(O[C@H]5CC[C@H](CN6CCN(Cc7cc8c(c(C(F)(F)F)c7)CN(c7cccc(C9(Cc%10nnc[nH]%10)COC9)c7)C8=O)CC6)CC5)CC4)c3F)C2=O)C(=O)N1] | 3-[4-fluoro-1-oxo-5-(4-{[(1r,4r)-4-[(4- {[3-oxo-2-(3-{3-[(4H-1,2,4-triazol-3- yl)methyl]oxetan-3-yl}phenyl)-7- (trifluoromethyl)-2,3-dihydro-1H- isoindol-5-yl]methyl}piperazin-1- yl)methyl]cyclohexyl]oxy}piperidin-1- yl)-2,3-dihydro-1H-isoindol-2- yl]piperidine-2,6-dione | Scheme D |
| 198 | 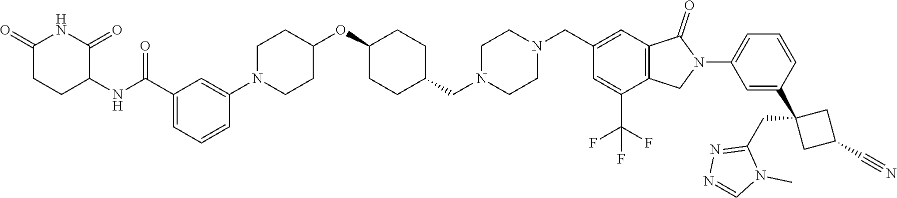 [SMILES: Cn1cnnc1C[C@]1(c2cccc(N3Cc4c(cc(CN5CCN(C[C@H]6CC[C@H](OC7CCN(c8cccc(C(=O)NC9CCC(=O)NC9=O)c8)CC7)CC6)CC5)cc4C(F)(F)F)C3=O)c2)C[C@H](C#N)C1] | N-(2,6-dioxopiperidin-3-yl)-3-(4- {[(1r,4r)-4-({4-[(3-oxo-2-{3-[(1r,3r)-3- cyano-1-[(4-methyl-4H-1,2,4-triazol-3- yl)methyl]cyclobutyl]phenyl}-7- (trifluoromethyl)-2,3-dihydro-1H- isoindol-5-yl)methyl]piperazin-1- yl}methyl)cyclohexyl]oxy}piperidin-1- yl)benzamide | Scheme D |
| 199 | 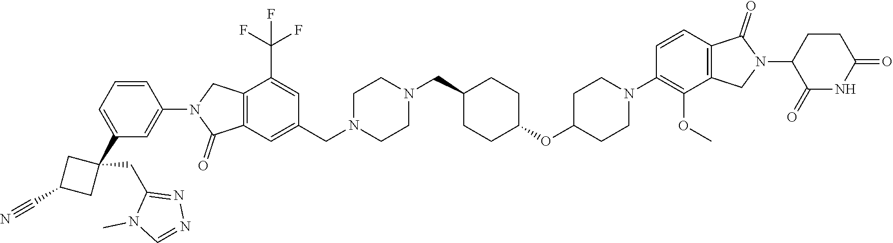 [SMILES: COc1c(N2CCC(O[C@H]3CC[C@H](CN4CCN(Cc5cc6c(c(C(F)(F)F)c5)CN(c5cccc([C@]7(Cc8nncn8C)C[C@@H](C#N)C7)c5)C6=O)CC4)CC3)CC2)ccc2c1CN(C1CCC(=O)NC1=O)C2=O] | (1r,3r)-3-[(4-methyl-4H-1,2,4-triazol-3- yl)methyl]-3-(3-{1-oxo-6-[(4-{[(1r,4r)- 4-({1-[2-(2,6-dioxopiperidin-3-yl)-4- methoxy-1-oxo-2,3-dihydro-1H- isoindol-5-yl]piperidin-4- yl}oxy)cyclohexyl]methyl}piperazin-1- yl)methyl]-4-(trifluoromethyl)-2,3- dihydro-1H-isoindol-2- yl}phenyl)cyclobutane-1-carbonitrile | Scheme D |
| 200 | 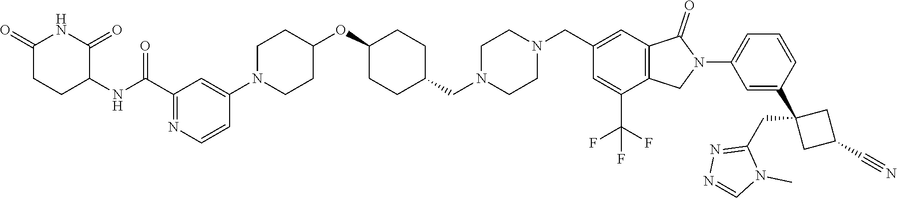 [SMILES: Cn1cnnc1C[C@]1(c2cccc(N3Cc4c(cc(CN5CCN(C[C@H]6CC[C@H](OC7CCN(c8ccnc(C(=O)NC9CCC(=O)NC9=O)c8)CC7)CC6)CC5)cc4C(F)(F)F)C3=O)c2)C[C@H](C#N)C1] | N-(2,6-dioxopiperidin-3-yl)-4-(4- {[(1r,4r)-4-({4-[(3-oxo-2-{3-[(1r,3r)-3- cyano-1-[(4-methyl-4H-1,2,4-triazol-3- yl)methyl]cyclobutyl]phenyl}-7- (trifluoromethyl)-2,3-dihydro-1H- isoindol-5-yl)methyl]piperazin-1- yl}methyl)cyclohexyl]oxy}piperidin-1- yl)pyridine-2-carboxamide | Scheme D |
| 201 | 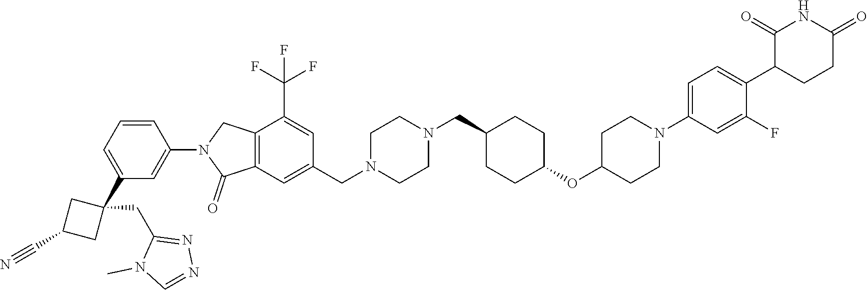 [SMILES: Cn1cnnc1C[C@]1(c2cccc(N3Cc4c(cc(CN5CCN(C[C@H]6CC[C@H](OC7CCN(c8ccc(C9CCC(=O)NC9=O)c(F)c8)CC7)CC6)CC5)cc4C(F)(F)F)C3=O)c2)C[C@H](C#N)C1] | (1r,3r)-3-[(4-methyl-4H-1,2,4-triazol-3- yl)methyl]-3-(3-{1-oxo-6-[(4-{[(1r,4r)- 4-({1-[4-(2,6-dioxopiperidin-3-yl)-3- fluorophenyl]piperidin-4- yl}oxy)cyclohexyl]methyl}piperazin-1- yl)methyl]-4-(trifluoromethyl)-2,3- dihydro-1H-isoindol-2- yl}phenyl)cyclobutane-1-carbonitrile | Scheme D |
| 202 | 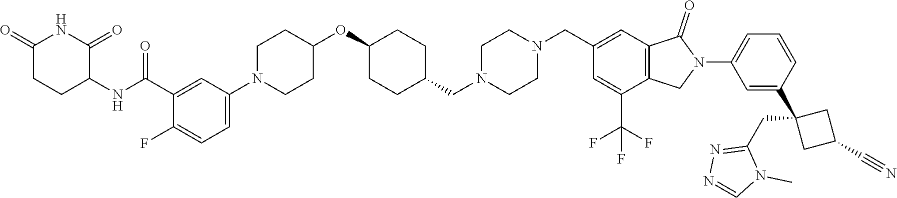 [SMILES: Cn1cnnc1C[C@]1(c2cccc(N3Cc4c(cc(CN5CCN(C[C@H]6CC[C@H](OC7CCN(c8ccc(F)c(C(=O)NC9CCC(=O)NC9=O)c8)CC7)CC6)CC5)cc4C(F)(F)F)C3=O)c2)C[C@H](C#N)C1] | N-(2,6-dioxopiperidin-3-yl)-2-fluoro-5- (4-{[(1r,4r)-4-({4-[(3-oxo-2-{3- [(1r,3r)-3-cyano-1-[(4-methyl-4H- 1,2,4-triazol-3- yl)methyl]cyclobutyl]phenyl}-7- (trifluoromethyl)-2,3-dihydro-1H- isoindol-5-yl)methyl]piperazin-1- yl}methyl)cyclohexyl]oxy}piperidin-1- yl)benzamide | Scheme D |
| 203 | 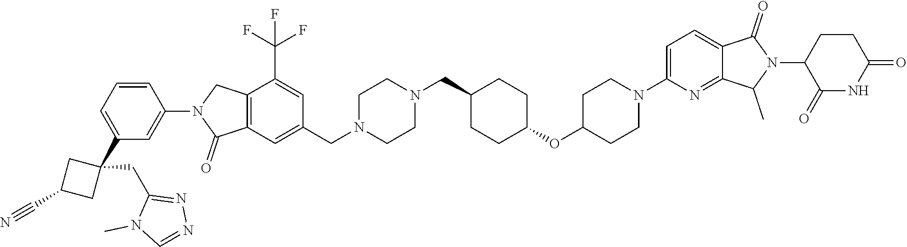 [SMILES: CC1c2nc(N3CCC(O[C@H]4CC[C@H](CN5CCN(Cc6cc7c(c(C(F)(F)F)c6)CN(c6cccc([C@]8(Cc9nncn9C)C[C@@H](C#N)C8)c6)C7=O)CC5)CC4)CC3)ccc2C(=O)N1C1CCC(=O)NC1=O] | (1r,3r)-3-[(4-methyl-4H-1,2,4-triazol-3- yl)methyl]-3-(3-{1-oxo-6-[(4-{[(1r,4r)- 4-({1-[6-(2,6-dioxopiperidin-3-yl)-7- methyl-5-oxo-5H,6H,7H-pyrrolo[3,4- b]pyridin-2-yl]piperidin-4- yl}oxy)cyclohexyl]methyl}piperazin-1- yl)methyl]-4-(trifluoromethyl)-2,3- dihydro-1H-isoindol-2- yl}phenyl)cyclobutane-1-carbonitrile | Scheme D |
| 204 | 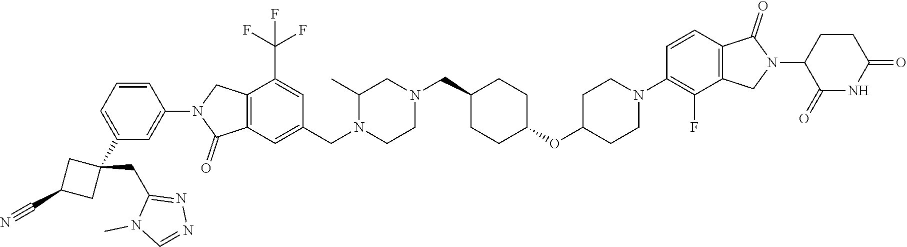 [SMILES: CC1CN(C[C@H]2CC[C@H](OC3CCN(c4ccc5c(c4F)CN(C4CCC(=O)NC4=O)C5=O)CC3)CC2)CCN1Cc1cc2c(c(C(F)(F)F)c1)CN(c1cccc([C@]3(Cc4nncn4C)C[C@@H](C#N)C3)c1)C2=O] | (1r,3r)-3-(3-{6-[(2-methyl-4-{[(1r,4r)- 4-({1-[2-(2,6-dioxopiperidin-3-yl)-4- fluoro-1-oxo-2,3-dihydro-1H-isoindol- 5-yl]piperidin-4- yl}oxy)cyclohexyl]methyl}piperazin-1- yl)methyl]-1-oxo-4-(trifluoromethyl)- 2,3-dihydro-1H-isoindol-2-yl}phenyl)- 3-[(4-methyl-4H-1,2,4-triazol-3- yl)methyl]cyclobutane-1-carbonitrile | Scheme D |
| 205 | 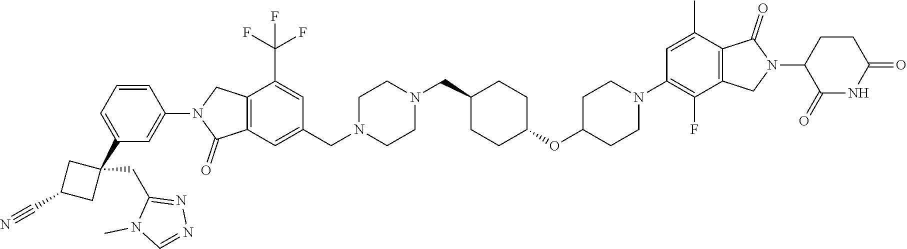 [SMILES: Cc1cc(N2CCC(O[C@H]3CC[C@H](CN4CCN(Cc5cc6c(c(C(F)(F)F)c5)CN(c5cccc([C@]7(Cc8nncn8C)C[C@@H](C#N)C7)c5)C6=O)CC4)CC3)CC2)c(F)c2c1C(=O)N(C1CCC(=O)NC1=O)C2] | (1r,3r)-3-[(4-methyl-4H-1,2,4-triazol-3- yl)methyl]-3-(3-{1-oxo-6-[(4-{[(1r,4r)- 4-({1-[2-(2,6-dioxopiperidin-3-yl)-4- fluoro-7-methyl-1-oxo-2,3-dihydro-1H- isoindol-5-yl]piperidin-4- yl}oxy)cyclohexyl]methyl}piperazin-1- yl)methyl]-4-(trifluoromethyl)-2,3- dihydro-1H-isoindol-2- yl}phenyl)cyclobutane-1-carbonitrile | Scheme D |
| 206 | 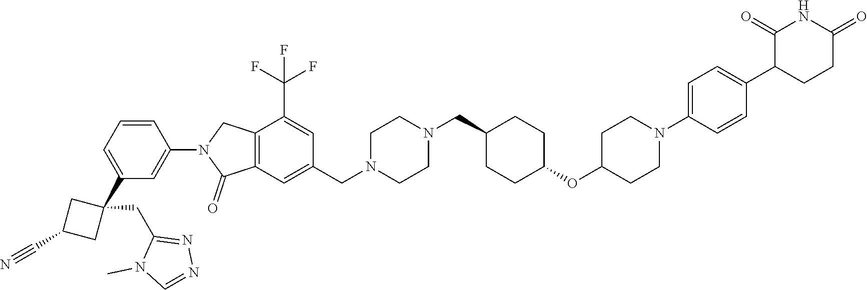 [SMILES: Cn1cnnc1C[C@]1(c2cccc(N3Cc4c(cc(CN5CCN(C[C@H]6CC[C@H](OC7CCN(c8ccc(C9CCC(=O)NC9=O)cc8)CC7)CC6)CC5)cc4C(F)(F)F)C3=O)c2)C[C@H](C#N)C1] | (1r,3r)-3-[(4-methyl-4H-1,2,4-triazol-3- yl)methyl]-3-(3-{1-oxo-6-[(4-{[(1r,4r)- 4-({1-[4-(2,6-dioxopiperidin-3- yl)phenyl]piperidin-4- yl}oxy)cyclohexyl]methyl}piperazin-1- yl)methyl]-4-(trifluoromethyl)-2,3- dihydro-1H-isoindol-2- yl}phenyl)cyclobutane-1-carbonitrile | Scheme D |
| 207 | 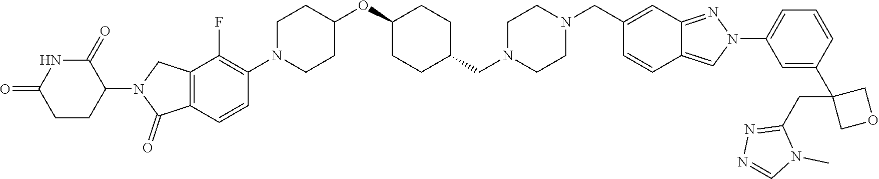 [SMILES: Cn1cnnc1CC1(c2cccc(-n3cc4ccc(CN5CCN(C[C@H]6CC[C@H](OC7CCN(c8ccc9c(c8F)CN(C8CCC(=O)NC8=O)C9=O)CC7)CC6)CC5)cc4n3)c2)COC1] | 3-[4-fluoro-1-oxo-5-(4-{[(1r,4r)-4-[(4- {[2-(3-{3-[(4-methyl-4H-1,2,4-triazol- 3-yl)methyl]oxetan-3-yl}phenyl)-2H- indazol-6-yl]methyl}piperazin-1- yl)methyl]cyclohexyl]oxy}piperidin-1- yl)-2,3-dihydro-1H-isoindol-2- yl]piperidine-2,6-dione | Scheme D |
| 208 | 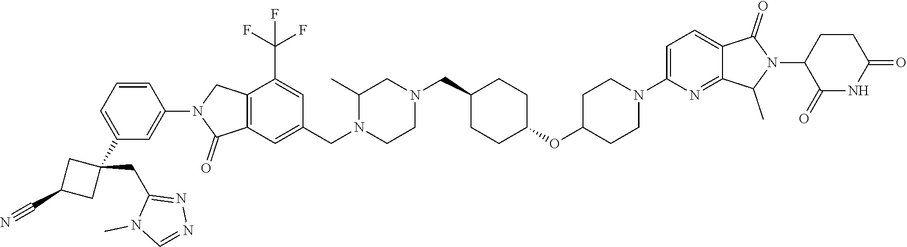 [SMILES: CC1CN(C[C@H]2CC[C@H](OC3CCN(c4ccc5c(n4)C(C)N(C4CCC(=O)NC4=O)C5=O)CC3)CC2)CCN1Cc1cc2c(c(C(F)(F)F)c1)CN(c1cccc([C@]3(Cc4nncn4C)C[C@@H](C#N)C3)c1)C2=O] | (1r,3r)-3-(3-{6-[(2-methyl-4-{[(1r,4r)- 4-({1-[6-(2,6-dioxopiperidin-3-yl)-7- methyl-5-oxo-5H,6H,7H-pyrrolo[3,4- b]pyridin-2-yl]piperidin-4- yl}oxy)cyclohexyl]methyl}piperazin-1- yl)methyl]-1-oxo-4-(trifluoromethyl)- 2,3-dihydro-1H-isoindol-2-yl}phenyl)- 3-[(4-methyl-4H-1,2,4-triazol-3- yl)methyl]cyclobutane-1-carbonitrile | Scheme D |
| 209 | 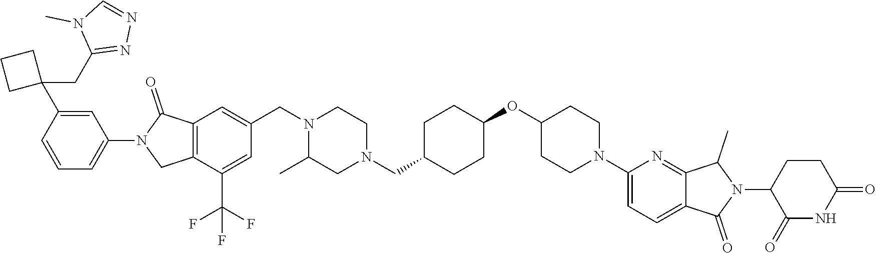 [SMILES: CC1CN(C[C@H]2CC[C@H](OC3CCN(c4ccc5c(n4)C(C)N(C4CCC(=O)NC4=O)C5=O)CC3)CC2)CCN1Cc1cc2c(c(C(F)(F)F)c1)CN(c1cccc(C3(Cc4nncn4C)CCC3)c1)C2=O] | 3-[7-methyl-5-oxo-2-(4-{[(1r,4r)-4-[(3- methyl-4-{[2-(3-{1-[(4-methyl-4H- 1,2,4-triazol-3- yl)methyl]cyclobutyl}phenyl)-3-oxo-7- (trifluoromethyl)-2,3-dihydro-1H- isoindol-5-yl]methyl}piperazin-1- yl)methyl]cyclohexyl]oxy}piperidin-1- yl)-5H,6H,7H-pyrrolo[3,4-b]pyridin-6- yl]piperidine-2,6-dione | Scheme D |
| 210 | 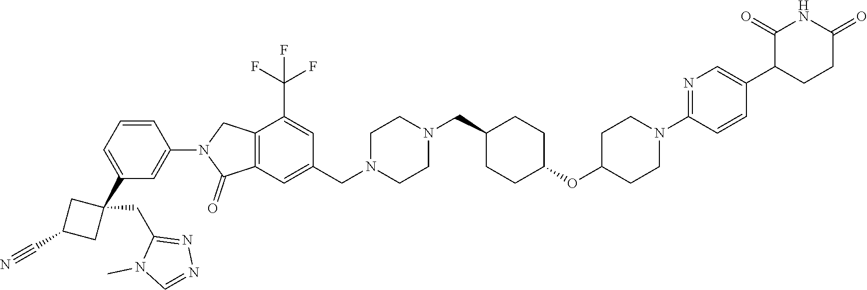 [SMILES: Cn1cnnc1C[C@]1(c2cccc(N3Cc4c(cc(CN5CCN(C[C@H]6CC[C@H](OC7CCN(c8ccc(C9CCC(=O)NC9=O)cn8)CC7)CC6)CC5)cc4C(F)(F)F)C3=O)c2)C[C@H](C#N)C1] | (1r,3r)-3-[(4-methyl-4H-1,2,4-triazol-3- yl)methyl]-3-(3-{1-oxo-6-[(4-{[(1r,4r)- 4-({1-[5-(2,6-dioxopiperidin-3- yl)pyridin-2-yl]piperidin-4- yl}oxy)cyclohexyl]methyl}piperazin-1- yl)methyl]-4-(trifluoromethyl)-2,3- dihydro-1H-isoindol-2- yl}phenyl)cyclobutane-1-carbonitrile | Scheme D |
| 211 | 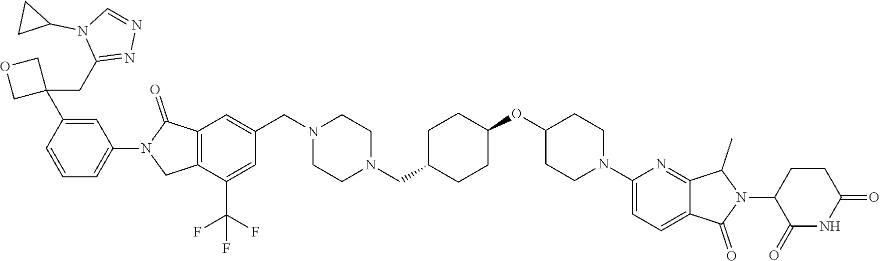 [SMILES: CC1c2nc(N3CCC(O[C@H]4CC[C@H](CN5CCN(Cc6cc7c(c(C(F)(F)F)c6)CN(c6cccc(C8(Cc9nncn9C9CC9)COC8)c6)C7=O)CC5)CC4)CC3)ccc2C(=O)N1C1CCC(=O)NC1=O] | 3-[7-methyl-5-oxo-2-(4-{[(1r,4r)-4-[(4- {[2-(3-{3-[(4-cyclopropyl-4H-1,2,4- triazol-3-yl)methyl]oxetan-3- yl}phenyl)-3-oxo-7-(trifluoromethyl)- 2,3-dihydro-1H-isoindol-5- yl]methyl}piperazin-1- yl)methyl]cyclohexyl]oxy}piperidin-1- yl)-5H,6H,7H-pyrrolo[3,4-b]pyridin-6- yl]piperidine-2,6-dione | Scheme D |
| 212 | 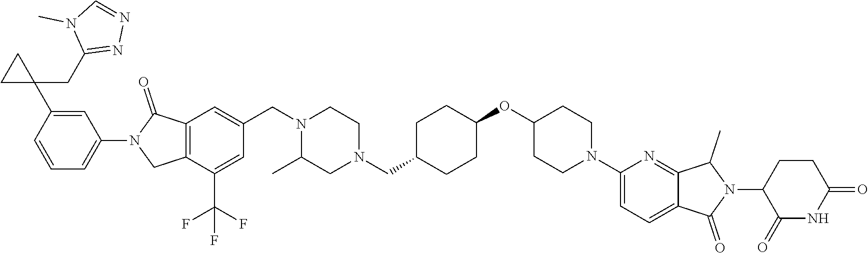 [SMILES: CC1CN(C[C@H]2CC[C@H](OC3CCN(c4ccc5c(n4)C(C)N(C4CCC(=O)NC4=O)C5=O)CC3)CC2)CCN1Cc1cc2c(c(C(F)(F)F)c1)CN(c1cccc(C3(Cc4nncn4C)CC3)c1)C2=O] | 3-[7-methyl-5-oxo-2-(4-{[(1r,4r)-4-[(3- methyl-4-{[2-(3-{1-[(4-methyl-4H- 1,2,4-triazol-3- yl)methyl]cyclopropyl}phenyl)-3-oxo- 7-(trifluoromethyl)-2,3-dihydro-1H- isoindol-5-yl]methyl}piperazin-1- yl)methyl]cyclohexyl]oxy}piperidin-1- yl)-5H,6H,7H-pyrrolo[3,4-b]pyridin-6- yl]piperidine-2,6-dione | Scheme D |
| 213 | 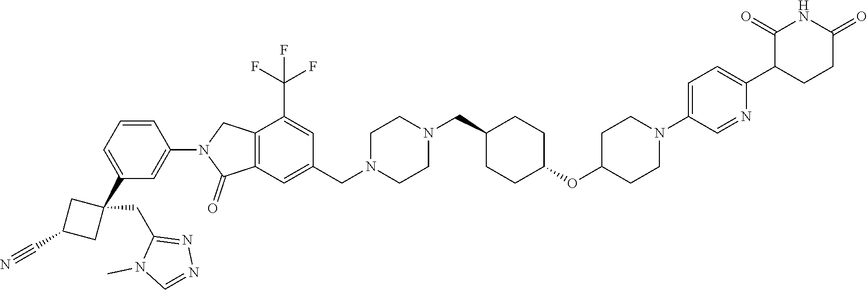 [SMILES: Cn1cnnc1C[C@]1(c2cccc(N3Cc4c(cc(CN5CCN(C[C@H]6CC[C@H](OC7CCN(c8ccc(C9CCC(=O)NC9=O)nc8)CC7)CC6)CC5)cc4C(F)(F)F)C3=O)c2)C[C@H](C#N)C1] | (1r,3r)-3-[(4-methyl-4H-1,2,4-triazol-3- yl)methyl]-3-(3-{1-oxo-6-[(4-{[(1r,4r)- 4-({1-[6-(2,6-dioxopiperidin-3- yl)pyridin-3-yl]piperidin-4- yl}oxy)cyclohexyl]methyl}piperazin-1- yl)methyl]-4-(trifluoromethyl)-2,3- dihydro-1H-isoindol-2- yl}phenyl)cyclobutane-1-carbonitrile | Scheme D |
| 214 | 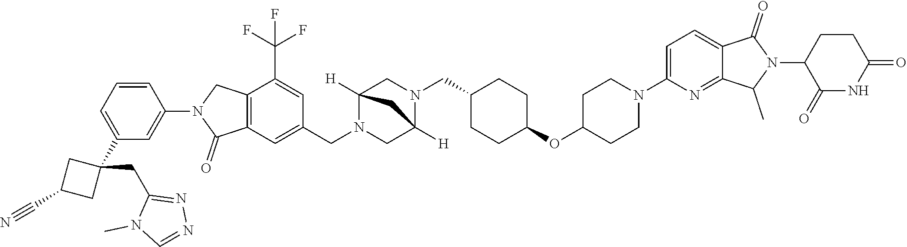 [SMILES: CC1c2nc(N3CCC(O[C@H]4CC[C@H](CN5C[C@@H]6C[C@H]5CN6Cc5cc6c(c(C(F)(F)F)c5)CN(c5cccc([C@]7(Cc8nncn8C)C[C@H](C#N)C7)c5)C6=O)CC4)CC3)ccc2C(=O)N1C1CCC(=O)NC1=O] | (1r,3r)-3-[(4-methyl-4H-1,2,4-triazol-3- yl)methyl]-3-[3-(1-oxo-6-{[(1S,4S)-5- {[(1r,4r)-4-({1-[6-(2,6-dioxopiperidin- 3-yl)-7-methyl-5-oxo-5H,6H,7H- pyrrolo[3,4-b]pyridin-2-yl]piperidin-4- yl}oxy)cyclohexyl]methyl}-2,5- diazabicyclo[2.2.1]heptan-2- yl]methyl}-4-(trifluoromethyl)-2,3- dihydro-1H-isoindol-2- yl)phenyl]cyclobutane-1-carbonitrile | Scheme D |
| 215 | 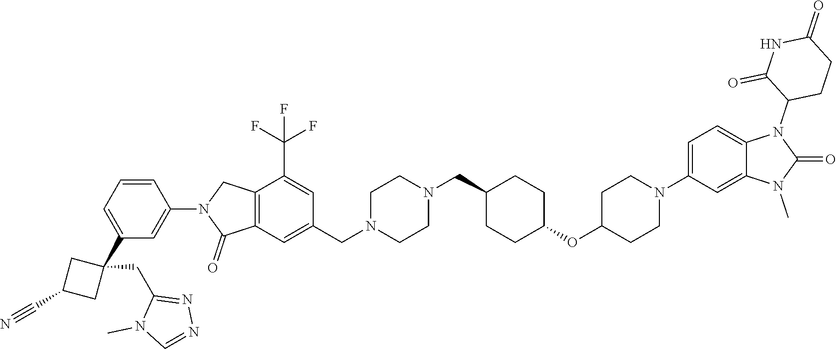 [SMILES: Cn1cnnc1C[C@]1(c2cccc(N3Cc4c(cc(CN5CCN(C[C@H]6CC[C@H](OC7CCN(c8ccc9c(c8)n(C)c(=O)n9C8CCC(=O)NC8=O)CC7)CC6)CC5)cc4C(F)(F)F)C3=O)c2)C[C@H](C#N)C1] | (1r,3r)-3-[(4-methyl-4H-1,2,4-triazol-3- yl)methyl]-3-(3-{1-oxo-6-[(4-{[(1r,4r)- 4-({1-[1-(2,6-dioxopiperidin-3-yl)-3- methyl-2-oxo-2,3-dihydro-1H-1,3- benzodiazol-5-yl]piperidin-4- yl}oxy)cyclohexyl]methyl}piperazin-1- yl)methyl]-4-(trifluoromethyl)-2,3- dihydro-1H-isoindol-2- yl}phenyl)cyclobutane-1-carbonitrile | Scheme D |
| 216 | 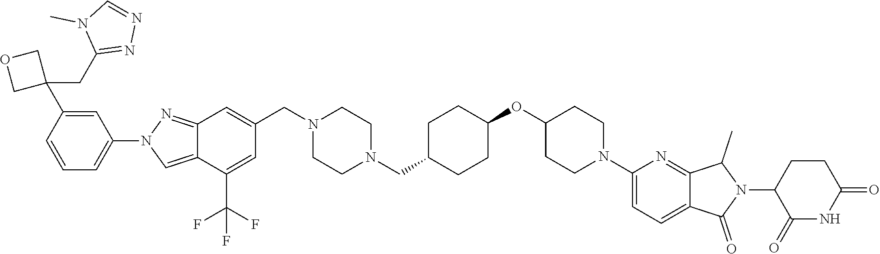 [SMILES: CC1c2nc(N3CCC(O[C@H]4CC[C@H](CN5CCN(Cc6cc(C(F)(F)F)c7cn(-c8cccc(C9(Cc%10nncn%10C)COC9)c8)nc7c6)CC5)CC4)CC3)ccc2C(=O)N1C1CCC(=O)NC1=O] | 3-[7-methyl-5-oxo-2-(4-{[(1r,4r)-4-[(4- {[2-(3-{3-[(4-methyl-4H-1,2,4-triazol- 3-yl)methyl]oxetan-3-yl}phenyl)-4- (trifluoromethyl)-2H-indazol-6- yl]methyl}piperazin-1- yl)methyl]cyclohexyl]oxy}piperidin-1- yl)-5H,6H,7H-pyrrolo[3,4-b]pyridin-6- yl]piperidine-2,6-dione | Scheme D |
| 217 | 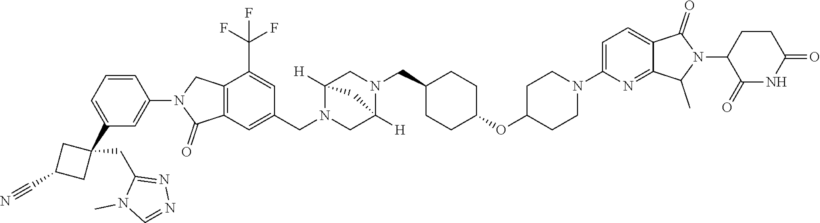 [SMILES: CC1c2nc(N3CCC(O[C@H]4CC[C@H](CN5C[C@H]6C[C@@H]5CN6Cc5cc6c(c(C(F)(F)F)c5)CN(c5cccc([C@]7(Cc8nncn8C)C[C@@H](C#N)C7)c5)C6=O)CC4)CC3)ccc2C(=O)N1C1CCC(=O)NC1=O] | (1r,3r)-3-[(4-methyl-4H-1,2,4-triazol-3- yl)methyl]-3-[3-(1-oxo-6-{[(1R,4R)-5- {[(1r,4r)-4-({1-[6-(2,6-dioxopiperidin- 3-yl)-7-methyl-5-oxo-5H,6H,7H- pyrrolo[3,4-b]pyridin-2-yl]piperidin-4- yl}oxy)cyclohexyl]methyl}-2,5- diazabicyclo[2.2.1]heptan-2- yl]methyl}-4-(trifluoromethyl)-2,3- dihydro-1H-isoindol-2- yl)phenyl]cyclobutane-1-carbonitrile | Scheme D |
| 218 | 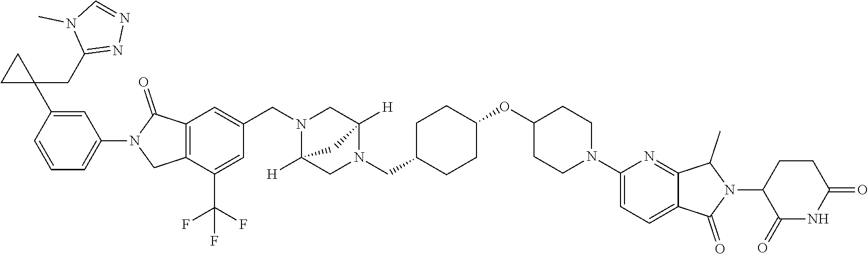 [SMILES: CC1c2nc(N3CCC(O[C@H]4CC[C@@H](CN5C[C@@H]6C[C@H]5CN6Cc5cc6c(c(C(F)(F)F)c5)CN(c5cccc(C7(Cc8nncn8C)CC7)c5)C6=O)CC4)CC3)ccc2C(=O)N1C1CCC(=O)NC1=O] | 3-[7-methyl-5-oxo-2-(4-{[(1r,4r)-4- {[(1S,4S)-5-{[2-(3-{1-[(4-methyl-4H- 1,2,4-triazol-3- yl)methyl]cyclopropyl}phenyl)-3-oxo- 7-(trifluoromethyl)-2,3-dihydro-1H- isoindol-5-yl]methyl}-2,5- diazabicyclo[2.2.1]heptan-2- yl]methyl}cyclohexyl]oxy}piperidin-1- yl)-5H,6H,7H-pyrrolo[3,4-b]pyridin-6- yl]piperidine-2,6-dione | Scheme D |
| 219 | 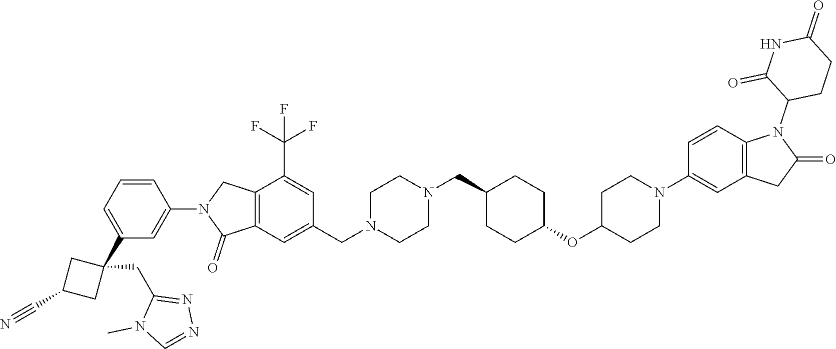 [SMILES: Cn1cnnc1C[C@]1(c2cccc(N3Cc4c(cc(CN5CCN(C[C@H]6CC[C@H](OC7CCN(c8ccc9c(c8)CC(=O)N9C8CCC(=O)NC8=O)CC7)CC6)CC5)cc4C(F)(F)F)C3=O)c2)C[C@H](C#N)C1] | (1r,3r)-3-[(4-methyl-4H-1,2,4-triazol-3- yl)methyl]-3-(3-{1-oxo-6-[(4-{[(1r,4r)- 4-({1-[1-(2,6-dioxopiperidin-3-yl)-2- oxo-2,3-dihydro-1H-indol-5- yl]piperidin-4- yl}oxy)cyclohexyl]methyl}piperazin-1- yl)methyl]-4-(trifluoromethyl)-2,3- dihydro-1H-isoindol-2- yl}phenyl)cyclobutane-1-carbonitrile | Scheme D |
| 220 | 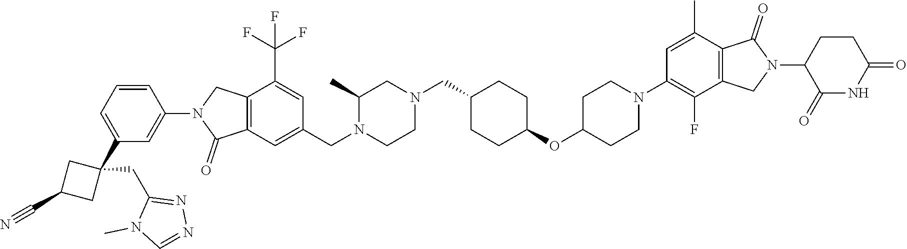 [SMILES: Cc1cc(N2CCC(O[C@H]3CC[C@H](CN4CCN(Cc5cc6c(c(C(F)(F)F)c5)CN(c5cccc([C@]7(Cc8nncn8C)C[C@H](C#N)C7)c5)C6=O)[C@@H](C)C4)CC3)CC2)c(F)c2c1C(=O)N(C1CCC(=O)NC1=O)C2] | (1r,3r)-3-[3-(6-{[(2S)-2-methyl-4- {[(1r,4r)-4-({1-[2-(2,6-dioxopiperidin- 3-yl)-4-fluoro-7-methyl-1-oxo-2,3- dihydro-1H-isoindol-5-yl]piperidin-4- yl}oxy)cyclohexyl]methyl}piperazin-1- yl]methyl}-1-oxo-4-(trifluoromethyl)- 2,3-dihydro-1H-isoindol-2-yl)phenyl]- 3-[(4-methyl-4H-1,2,4-triazol-3- yl)methyl]cyclobutane-1-carbonitrile | Scheme D |
| 221 | 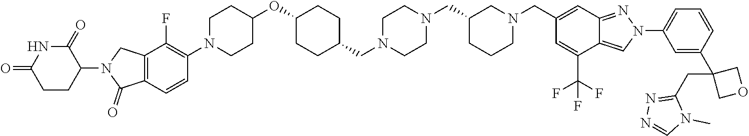 [SMILES: Cn1cnnc1CC1(c2cccc(-n3cc4c(C(F)(F)F)cc(CN5CCC[C@H](CN6CCN(C[C@H]7CC[C@@H](OC8CCN(c9ccc%10c(c9F)CN(C9CCC(=O)NC9=O)C%10=O)CC8)CC7)CC6)C5)cc4n3)c2)COC1] | 3-[4-fluoro-1-oxo-5-(4-{[(1r,4r)-4-[(4- {[(3S)-1-{[2-(3-{3-[(4-methyl-4H- 1,2,4-triazol-3-yl)methyl]oxetan-3- yl}phenyl)-4-(trifluoromethyl)-2H- indazol-6-yl]methyl}piperidin-3- yl]methyl}piperazin-1- yl)methyl]cyclohexyl]oxy}piperidin-1- yl)-2,3-dihydro-1H-isoindol-2- yl]piperidine-2,6-dione | Scheme D |
| 222 | 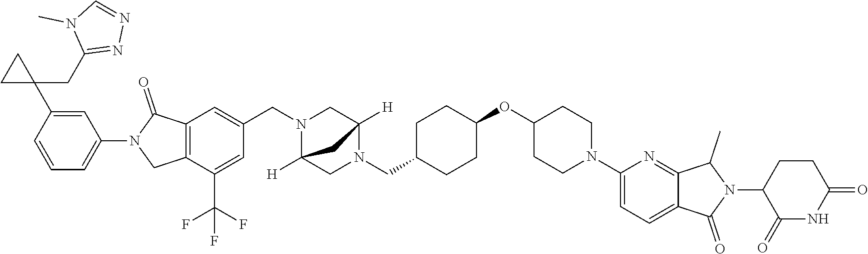 [SMILES: CC1c2nc(N3CCC(O[C@H]4CC[C@H](CN5C[C@H]6C[C@@H]5CN6Cc5cc6c(c(C(F)(F)F)c5)CN(c5cccc(C7(Cc8nncn8C)CC7)c5)C6=O)CC4)CC3)ccc2C(=O)N1C1CCC(=O)NC1=O] | 3-[7-methyl-5-oxo-2-(4-{[(1r,4r)-4- {[(1R,4R)-5-{[2-(3-{1-[(4-methyl-4H- 1,2,4-triazol-3- yl)methyl]cyclopropyl}phenyl)-3-oxo- 7-(trifluoromethyl)-2,3-dihydro-1H- isoindol-5-yl]methyl}-2,5- diazabicyclo[2.2.1]heptan-2- yl]methyl}cyclohexyl]oxy}piperidin-1- yl)-5H,6H,7H-pyrrolo[3,4-b]pyridin-6- yl]piperidine-2,6-dione | Scheme D |
| 223 | 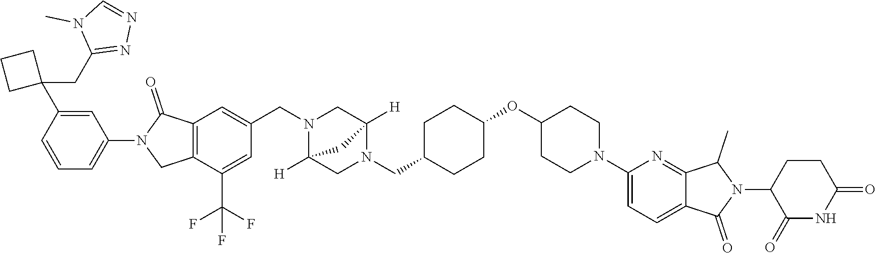 [SMILES: CC1c2nc(N3CCC(O[C@H]4CC[C@@H](CN5C[C@@H]6C[C@H]5CN6Cc5cc6c(c(C(F)(F)F)c5)CN(c5cccc(C7(Cc8nncn8C)CCC7)c5)C6=O)CC4)CC3)ccc2C(=O)N1C1CCC(=O)NC1=O] | 3-[7-methyl-5-oxo-2-(4-{[(1r,4r)-4- {[(1S,4S)-5-{[2-(3-{1-[(4-methyl-4H- 1,2,4-triazol-3- yl)methyl]cyclobutyl}phenyl)-3-oxo-7- (trifluoromethyl)-2,3-dihydro-1H- isoindol-5-yl]methyl}-2,5- diazabicyclo[2.2.1]heptan-2- yl]methyl}cyclohexyl]oxy}piperidin-1- yl)-5H,6H,7H-pyrrolo[3,4-b]pyridin-6- yl]piperidine-2,6-dione | Scheme D |
| 224 | 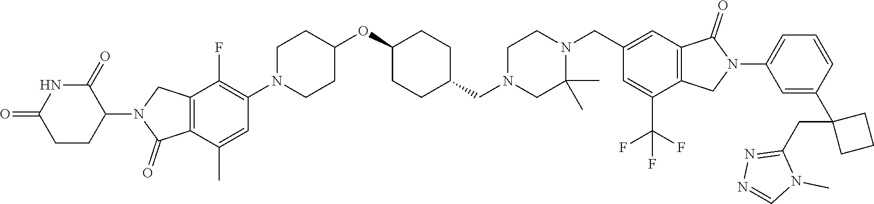 [SMILES: Cc1cc(N2CCC(O[C@H]3CC[C@H](CN4CCN(Cc5cc6c(c(C(F)(F)F)c5)CN(c5cccc(C7(Cc8nncn8C)CCC7)c5)C6=O)C(C)(C)C4)CC3)CC2)c(F)c2c1C(=O)N(C1CCC(=O)NC1=O)C2] | 3-[4-fluoro-7-methyl-1-oxo-5-(4- {[(1r,4r)-4-[(3,3-dimethyl-4-{[2-(3-{1- [(4-methyl-4H-1,2,4-triazol-3- yl)methyl]cyclobutyl}phenyl)-3-oxo-7- (trifluoromethyl)-2,3-dihydro-1H- isoindol-5-yl]methyl}piperazin-1- yl)methyl]cyclohexyl]oxy}piperidin-1- yl)-2,3-dihydro-1H-isoindol-2- yl]piperidine-2,6-dione | Scheme D |
| 225 | 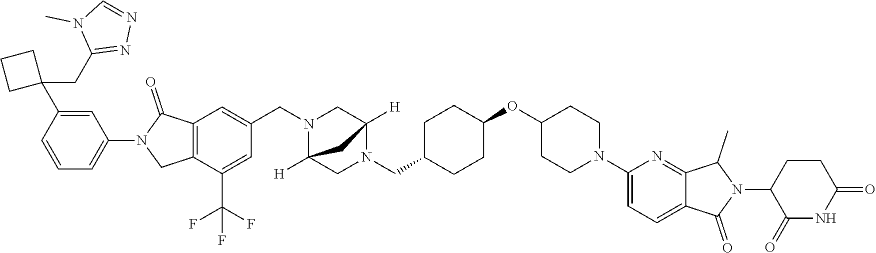 [SMILES: CC1c2nc(N3CCC(O[C@H]4CC[C@H](CN5C[C@H]6C[C@@H]5CN6Cc5cc6c(c(C(F)(F)F)c5)CN(c5cccc(C7(Cc8nncn8C)CCC7)c5)C6=O)CC4)CC3)ccc2C(=O)N1C1CCC(=O)NC1=O] | 3-[7-methyl-5-oxo-2-(4-{[(1r,4r)-4- {[(1R,4R)-5-{[2-(3-{1-[(4-methyl-4H- 1,2,4-triazol-3- yl)methyl]cyclobutyl}phenyl)-3-oxo-7- (trifluoromethyl)-2,3-dihydro-1H- isoindol-5-yl]methyl}-2,5- diazabicyclo[2.2.1]heptan-2- yl]methyl}cyclohexyl]oxy}piperidin-1- yl)-5H,6H,7H-pyrrolo[3,4-b]pyridin-6- yl]piperidine-2,6-dione | Scheme D |
| 226 | 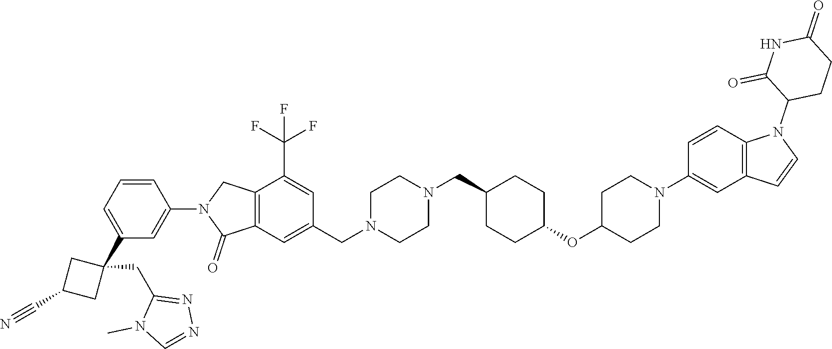 [SMILES: Cn1cnnc1C[C@]1(c2cccc(N3Cc4c(cc(CN5CCN(C[C@H]6CC[C@H](OC7CCN(c8ccc9c(ccn9C9CCC(=O)NC9=O)c8)CC7)CC6)CC5)cc4C(F)(F)F)C3=O)c2)C[C@H](C#N)C1] | (1r,3r)-3-[(4-methyl-4H-1,2,4-triazol-3- yl)methyl]-3-(3-{1-oxo-6-[(4-{[(1r,4r)- 4-({1-[1-(2,6-dioxopiperidin-3-yl)-1H- indol-5-yl]piperidin-4- yl}oxy)cyclohexyl]methyl}piperazin-1- yl)methyl]-4-(trifluoromethyl)-2,3- dihydro-1H-isoindol-2- yl}phenyl)cyclobutane-1-carbonitrile | Scheme D |
| 227 | 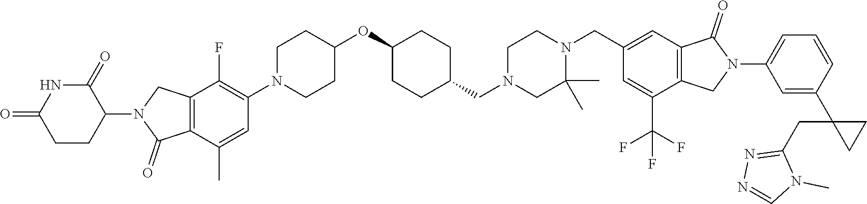 [SMILES: Cc1cc(N2CCC(O[C@H]3CC[C@H](CN4CCN(Cc5cc6c(c(C(F)(F)F)c5)CN(c5cccc(C7(Cc8nncn8C)CC7)c5)C6=O)C(C)(C)C4)CC3)CC2)c(F)c2c1C(=O)N(C1CCC(=O)NC1=O)C2] | 3-[4-fluoro-7-methyl-1-oxo-5-(4- {[(1r,4r)-4-[(3,3-dimethyl-4-{[2-(3-{1- [(4-methyl-4H-1,2,4-triazol-3- yl)methyl]cyclopropyl}phenyl)-3-oxo- 7-(trifluoromethyl)-2,3-dihydro-1H- isoindol-5-yl]methyl}piperazin-1- yl)methyl]cyclohexyl]oxy}piperidin-1- yl)-2,3-dihydro-1H-isoindol-2- yl]piperidine-2,6-dione | Scheme D |
| 228 | 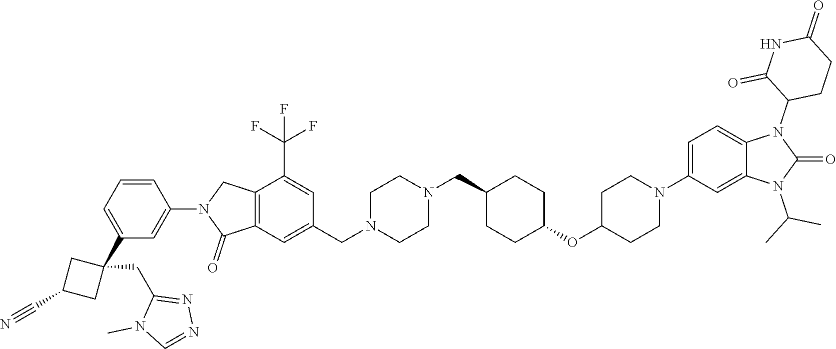 [SMILES: CC(C)n1c(=O)n(C2CCC(=O)NC2=O)c2ccc(N3CCC(O[C@H]4CC[C@H](CN5CCN(Cc6cc7c(c(C(F)(F)F)c6)CN(c6cccc([C@]8(Cc9nncn9C)C[C@@H](C#N)C8)c6)C7=O)CC5)CC4)CC3)cc21] | (1r,3r)-3-[(4-methyl-4H-1,2,4-triazol-3- yl)methyl]-3-(3-{1-oxo-6-[(4-{[(1r,4r)- 4-({1-[1-(2,6-dioxopiperidin-3-yl)-2- oxo-3-(propan-2-yl)-2,3-dihydro-1H- 1,3-benzodiazol-5-yl]piperidin-4- yl}oxy)cyclohexyl]methyl}piperazin-1- yl)methyl]-4-(trifluoromethyl)-2,3- dihydro-1H-isoindol-2- yl}phenyl)cyclobutane-1-carbonitrile | Scheme D |
| 229 | 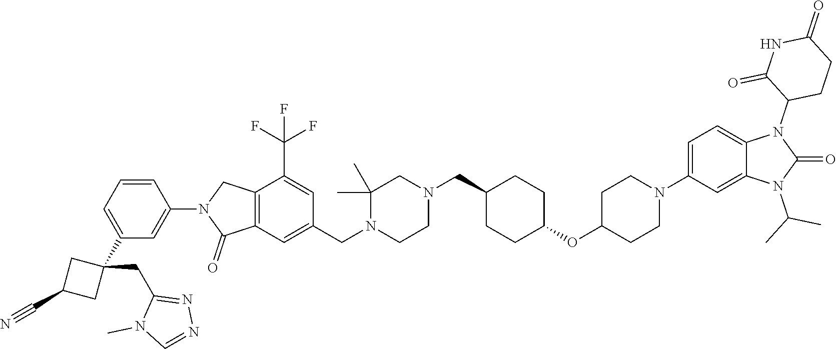 [SMILES: CC(C)n1c(=O)n(C2CCC(=O)NC2=O)c2ccc(N3CCC(O[C@H]4CC[C@H](CN5CCN(Cc6cc7c(c(C(F)(F)F)c6)CN(c6cccc([C@]8(Cc9nncn9C)C[C@@H](C#N)C8)c6)C7=O)C(C)(C)C5)CC4)CC3)cc21] | (1r,3r)-3-(3-{6-[(2,2-dimethyl-4- {[(1r,4r)-4-({1-[1-(2,6-dioxopiperidin- 3-yl)-2-oxo-3-(propan-2-yl)-2,3- dihydro-1H-1,3-benzodiazol-5- yl]piperidin-4- yl}oxy)cyclohexyl]methyl}piperazin-1- yl)methyl]-1-oxo-4-(trifluoromethyl)- 2,3-dihydro-1H-isoindol-2-yl}phenyl)- 3-[(4-methyl-4H-1,2,4-triazol-3- yl)methyl]cyclobutane-1-carbonitrile | Scheme D |
| 230 | 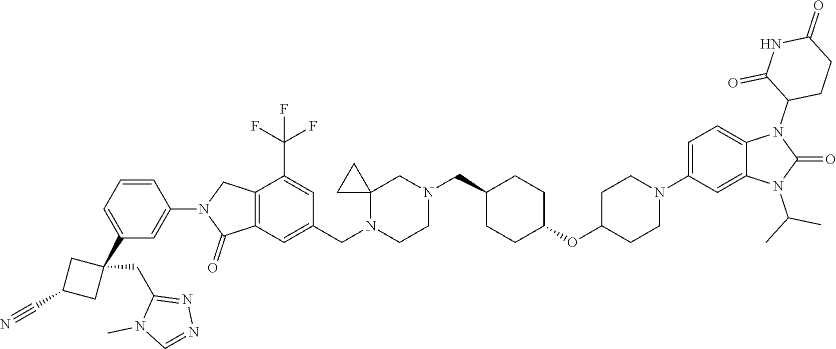 [SMILES: CC(C)n1c(=O)n(C2CCC(=O)NC2=O)c2ccc(N3CCC(O[C@H]4CC[C@H](CN5CCN(Cc6cc7c(c(C(F)(F)F)c6)CN(c6cccc([C@]8(Cc9nncn9C)C[C@@H](C#N)C8)c6)C7=O)C6(CC6)C5)CC4)CC3)cc21] | (1r,3r)-3-[(4-methyl-4H-1,2,4-triazol-3- yl)methyl]-3-(3-{1-oxo-6-[(7-{[(1r,4r)- 4-({1-[1-(2,6-dioxopiperidin-3-yl)-2- oxo-3-(propan-2-yl)-2,3-dihydro-1H- 1,3-benzodiazol-5-yl]piperidin-4- yl}oxy)cyclohexyl]methyl}-4,7- diazaspiro[2.5]octan-4-yl)methyl]-4- (trifluoromethyl)-2,3-dihydro-1H- isoindol-2-yl}phenyl)cyclobutane-1- carbonitrile | Scheme D |
| 231 | 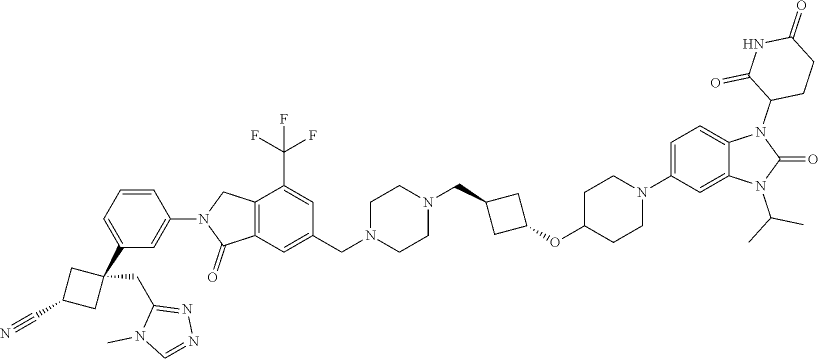 [SMILES: CC(C)n1c(=O)n(C2CCC(=O)NC2=O)c2ccc(N3CCC(O[C@H]4C[C@H](CN5CCN(Cc6cc7c(c(C(F)(F)F)c6)CN(c6cccc([C@]8(Cc9nncn9C)C[C@@H](C#N)C8)c6)C7=O)CC5)C4)CC3)cc21] | (1r,3r)-3-[(4-methyl-4H-1,2,4-triazol-3- yl)methyl]-3-(3-{1-oxo-6-[(4-{[(1r,3r)- 3-({1-[1-(2,6-dioxopiperidin-3-yl)-2- oxo-3-(propan-2-yl)-2,3-dihydro-1H- 1,3-benzodiazol-5-yl]piperidin-4- yl}oxy)cyclobutyl]methyl}piperazin-1- yl)methyl]-4-(trifluoromethyl)-2,3- dihydro-1H-isoindol-2- yl}phenyl)cyclobutane-1-carbonitrile | Scheme D |
| 232 | 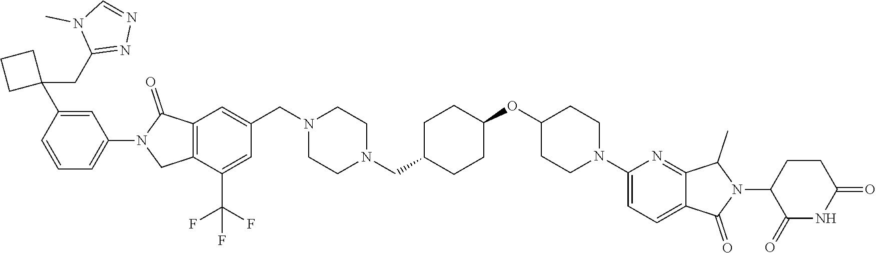 [SMILES: CC1c2nc(N3CCC(O[C@H]4CC[C@H](CN5CCN(Cc6cc7c(c(C(F)(F)F)c6)CN(c6cccc(C8(Cc9nncn9C)CCC8)c6)C7=O)CC5)CC4)CC3)ccc2C(=O)N1C1CCC(=O)NC1=O] | rac-3-[7-methyl-5-oxo-2-(4-{[(1r,4r)-4- [(4-{[2-(3-{1-[(4-methyl-4H-1,2,4- triazol-3- yl)methyl]cyclobutyl}phenyl)-3-oxo-7- (trifluoromethyl)-2,3-dihydro-1H- isoindol-5-yl]methyl}piperazin-1- yl)methyl]cyclohexyl]oxy}piperidin-1- yl)-5H,6H,7H-pyrrolo[3,4-b]pyridin-6- yl]piperidine-2,6-dione | Scheme E |
| 233 | 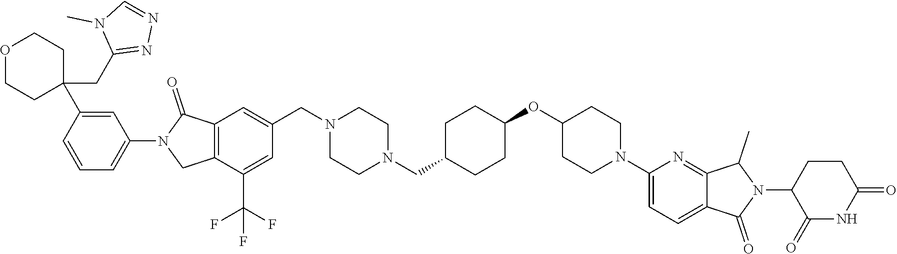 [SMILES: CC1c2nc(N3CCC(O[C@H]4CC[C@H](CN5CCN(Cc6cc7c(c(C(F)(F)F)c6)CN(c6cccc(C8(Cc9nncn9C)CCOCC8)c6)C7=O)CC5)CC4)CC3)ccc2C(=O)N1C1CCC(=O)NC1=O] | rac-3-[7-methyl-5-oxo-2-(4-{[(1r,4r)-4- [(4-{[2-(3-{4-[(4-methyl-4H-1,2,4- triazol-3-yl)methyl]oxan-4-yl}phenyl)- 3-oxo-7-(trifluoromethyl)-2,3-dihydro- 1H-isoindol-5-yl]methyl}piperazin-1- yl)methyl]cyclohexyl]oxy}piperidin-1- yl)-5H,6H,7H-pyrrolo[3,4-b]pyridin-6- yl]piperidine-2,6-dione | Scheme E |
| 234 | 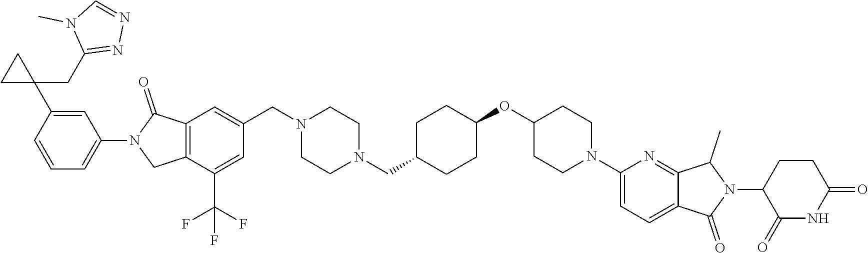 [SMILES: CC1c2nc(N3CCC(O[C@H]4CC[C@H](CN5CCN(Cc6cc7c(c(C(F)(F)F)c6)CN(c6cccc(C8(Cc9nncn9C)CC8)c6)C7=O)CC5)CC4)CC3)ccc2C(=O)N1C1CCC(=O)NC1=O] | rac-3-[7-methyl-5-oxo-2-(4-{[(1r,4r)-4- [(4-{[2-(3-{1-[(4-methyl-4H-1,2,4- triazol-3- yl)methyl]cyclopropyl}phenyl)-3-oxo- 7-(trifluoromethyl)-2,3-dihydro-1H- isoindol-5-yl]methyl}piperazin-1- yl)methyl]cyclohexyl]oxy}piperidin-1- yl)-5H,6H,7H-pyrrolo[3,4-b]pyridin-6- yl]piperidine-2,6-dione | Scheme E |
| 235 | 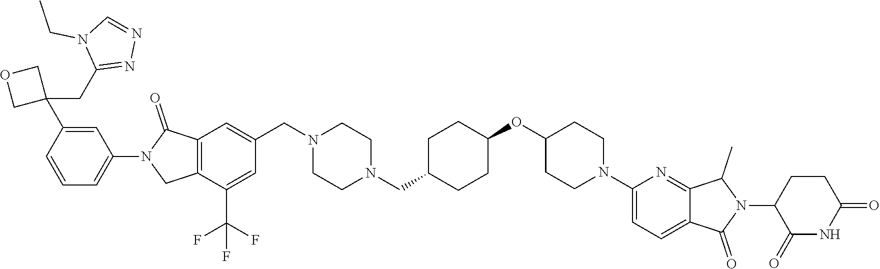 [SMILES: CCn1cnnc1CC1(c2cccc(N3Cc4c(cc(CN5CCN(C[C@H]6CC[C@H](OC7CCN(c8ccc9c(n8)C(C)N(C8CCC(=O)NC8=O)C9=O)CC7)CC6)CC5)cc4C(F)(F)F)C3=O)c2)COC1] | rac-3-[7-methyl-5-oxo-2-(4-{[(1r,4r)-4- [(4-{[2-(3-{3-[(4-ethyl-4H-1,2,4- triazol-3-yl)methyl]oxetan-3- yl}phenyl)-3-oxo-7-(trifluoromethyl)- 2,3-dihydro-1H-isoindol-5- yl]methyl}piperazin-1- yl)methyl]cyclohexyl]oxy}piperidin-1- yl)-5H,6H,7H-pyrrolo[3,4-b]pyridin-6- yl]piperidine-2,6-dione | Scheme E |
| 236 | 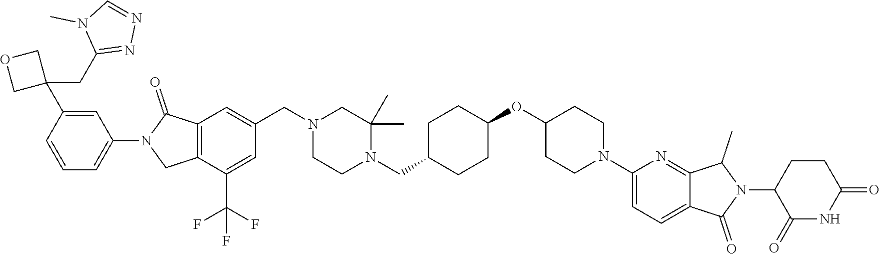 [SMILES: CC1c2nc(N3CCC(O[C@H]4CC[C@H](CN5CCN(Cc6cc7c(c(C(F)(F)F)c6)CN(c6cccc(C8(Cc9nncn9C)COC8)c6)C7=O)CC5(C)C)CC4)CC3)ccc2C(=O)N1C1CCC(=O)NC1=O] | rac-3-[7-methyl-5-oxo-2-(4-{[(1r,4r)-4- [(2,2-dimethyl-4-{[2-(3-{3-[(4-methyl- 4H-1,2,4-triazol-3-yl)methyl]oxetan-3- yl}phenyl)-3-oxo-7-(trifluoromethyl)- 2,3-dihydro-1H-isoindol-5- yl]methyl}piperazin-1- yl)methyl]cyclohexyl]oxy}piperidin-1- yl)-5H,6H,7H-pyrrolo[3,4-b]pyridin-6- yl]piperidine-2,6-dione | Scheme E |
| 237 | 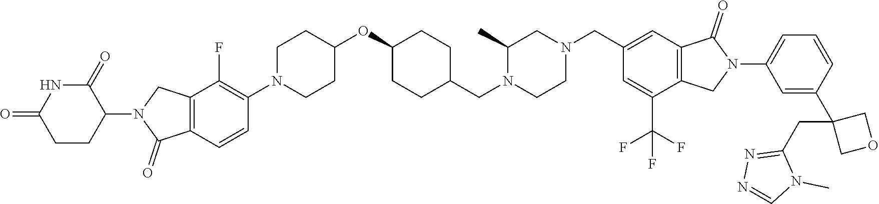 | 3-[4-fluoro-1-oxo-5-(4-{[(1rs,4rs)-4- {[(2S)-2-methyl-4-{[2-(3-{3-[(4- methyl-4H-1,2,4-triazol-3- yl)methyl]oxetan-3-yl}phenyl)-3-oxo- 7-(trifluoromethyl)-2,3-dihydro-1H- isoindol-5-yl]methyl}piperazin-1- yl]methyl}cyclohexyl]oxy}piperidin-1- yl)-2,3-dihydro-1H-isoindol-2- yl]piperidine-2,6-dione | Scheme E |
| 238 | 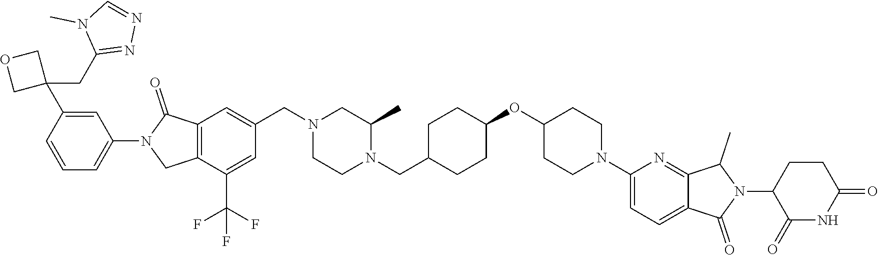 | 3-[7-methyl-5-oxo-2-(4-{[(1rs,4rs)-4- {[(2R)-2-methyl-4-{[2-(3-{3-[(4- methyl-4H-1,2,4-triazol-3- yl)methyl]oxetan-3-yl}phenyl)-3-oxo- 7-(trifluoromethyl)-2,3-dihydro-1H- isoindol-5-yl]methyl}piperazin-1- yl]methyl}cyclohexyl]oxy}piperidin-1- yl)-5H,6H,7H-pyrrolo[3,4-b]pyridin-6- yl]piperidine-2,6-dione | Scheme E |
| 239 | 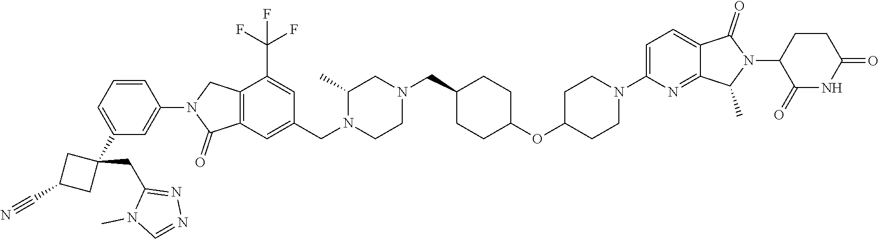 | (1rs&,3rs&)-3-[3-(6-{[(2R)-2-methyl- 4-{[(1rs,4rs)-4-({1-[(7R*)-6-(2,6- dioxopiperidin-3-yl)-7-methyl-5-oxo- 5H,6H,7H-pyrrolo[3,4-b]pyridin-2- yl]piperidin-4- yl}oxy)cyclohexyl]methyl}piperazin-1- yl]methyl}-1-oxo-4-(trifluoromethyl)- 2,3-dihydro-1H-isoindol-2-yl)phenyl]- 3-[(4-methyl-4H-1,2,4-triazol-3- yl)methyl]cyclobutane-1-carbonitrile | Scheme E |
| 240 | 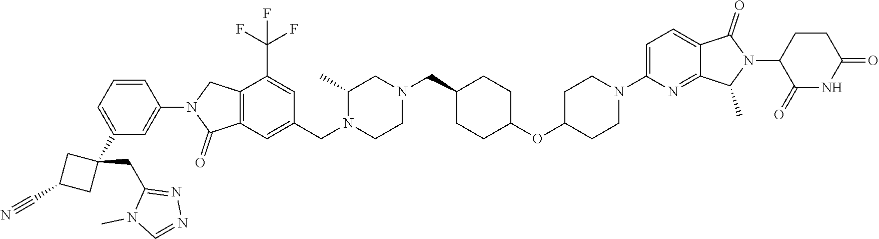 | (1rs&,3rs&)-3-[3-(6-{[(2R)-2-methyl- 4-{[(1rs,4rs)-4-({1-[(7R*)-6-(2,6- dioxopiperidin-3-yl)-7-methyl-5-oxo- 5H,6H,7H-pyrrolo[3,4-b]pyridin-2- yl]piperidin-4- yl}oxy)cyclohexyl]methyl}piperazin-1- yl]methyl}-1-oxo-4-(trifluoromethyl)- 2,3-dihydro-1H-isoindol-2-yl)phenyl]- 3-[(4-methyl-4H-1,2,4-triazol-3- yl)methyl]cyclobutane-1-carbonitrile | Scheme E |
| 241 | 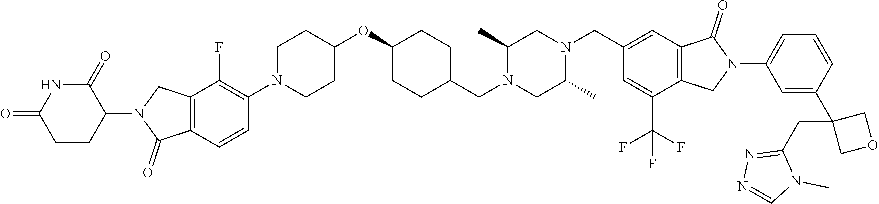 | 3-[4-fluoro-1-oxo-5-(4-{[(1rs,4rs)-4- {[(2S,5R)-2,5-dimethyl-4-{[2-(3-{3- [(4-methyl-4H-1,2,4-triazol-3- yl)methyl]oxetan-3-yl}phenyl)-3-oxo- 7-(trifluoromethyl)-2,3-dihydro-1H- isoindol-5-yl]methyl}piperazin-1- yl]methyl}cyclohexyl]oxy}piperidin-1- yl)-2,3-dihydro-1H-isoindol-2- yl]piperidine-2,6-dione | Scheme E |
| 242 | 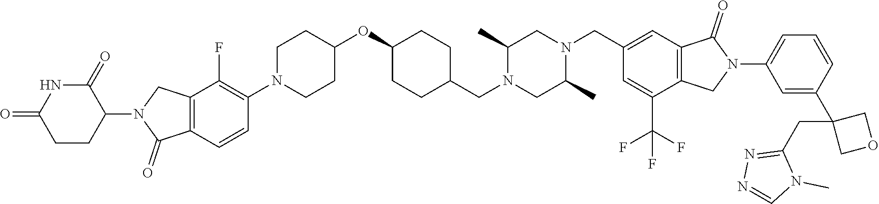 | 3-[4-fluoro-1-oxo-5-(4-{[(1rs,4rs)-4- {[(2S,5S)-2,5-dimethyl-4-{[2-(3-{3- [(4-methyl-4H-1,2,4-triazol-3- yl)methyl]oxetan-3-yl}phenyl)-3-oxo- 7-(trifluoromethyl)-2,3-dihydro-1H- isoindol-5-yl]methyl}piperazin-1- yl]methyl}cyclohexyl]oxy}piperidin-1- yl)-2,3-dihydro-1H-isoindol-2- yl]piperidine-2,6-dione | Scheme E |
| 243 | 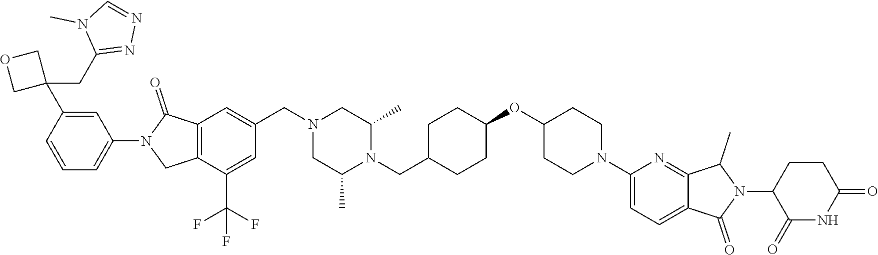 | 3-[7-methyl-5-oxo-2-(4-{[(1rs,4rs)-4- {[(2R,6S)-2,6-dimethyl-4-{[2-(3-{3- [(4-methyl-4H-1,2,4-triazol-3- yl)methyl]oxetan-3-yl}phenyl)-3-oxo- 7-(trifluoromethyl)-2,3-dihydro-1H- isoindol-5-yl]methyl}piperazin-1- yl]methyl}cyclohexyl]oxy}piperidin-1- yl)-5H,6H,7H-pyrrolo[3,4-b]pyridin-6- yl]piperidine-2,6-dione | Scheme E |
| 244 | 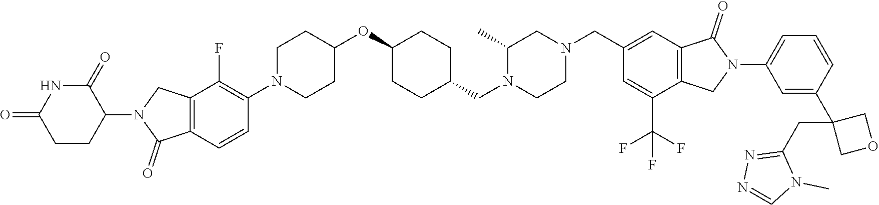 [SMILES: C[C@@H]1CN(Cc2cc3c(c(C(F)(F)F)c2)CN(c2cccc(C4(Cc5nncn5C)COC4)c2)C3=O)CCN1C[C@H]1CC[C@H](OC2CCN(c3ccc4c(c3F)CN(C3CCC(=O)NC3=O)C4=O)CC2)CC1] | 3-[4-fluoro-1-oxo-5-(4-{[(1r*,4r*)-4- {[(2R)-2-methyl-4-{[2-(3-{3-[(4- methyl-4H-1,2,4-triazol-3- yl)methyl]oxetan-3-yl}phenyl)-3-oxo- 7-(trifluoromethyl)-2,3-dihydro-1H- isoindol-5-yl]methyl}piperazin-1- yl]methyl}cyclohexyl]oxy}piperidin-1- yl)-2,3-dihydro-1H-isoindol-2- yl]piperidine-2,6-dione | Scheme E |
| 245 | 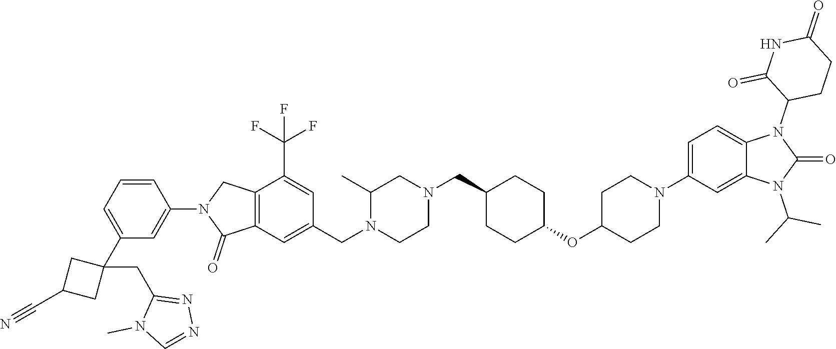 [SMILES: CC1CN(C[C@H]2CC[C@H](OC3CCN(c4ccc5c(c4)n(C(C)C)c(=O)n5C4CCC(=O)NC4=O)CC3)CC2)CCN1Cc1cc2c(c(C(F)(F)F)c1)CN(c1cccc(C3(Cc4nncn4C)CC(C#N)C3)c1)C2=O] | (1r**,3r**)-3-(3-{6-[(2-methyl-4- {[(1r*,4r*)-4-({1-[1-(2,6- dioxopiperidin-3-yl)-2-oxo-3-(propan- 2-yl)-2,3-dihydro-1H-1,3-benzodiazol- 5-yl]piperidin-4- yl}oxy)cyclohexyl]methyl}piperazin-1- yl)methyl]-1-oxo-4-(trifluoromethyl)- 2,3-dihydro-1H-isoindol-2-yl}phenyl)- 3-[(4-methyl-4H-1,2,4-triazol-3- yl)methyl]cyclobutane-1-carbonitrile | Scheme E |
| 246 | 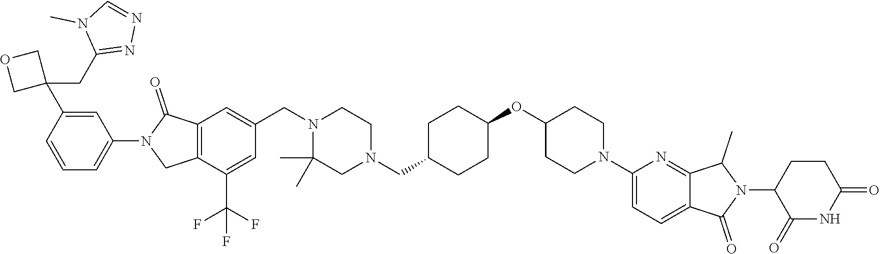 [SMILES: CC1c2nc(N3CCC(O[C@H]4CC[C@H](CN5CCN(Cc6cc7c(c(C(F)(F)F)c6)CN(c6cccc(C8(Cc9nncn9C)COC8)c6)C7=O)C(C)(C)C5)CC4)CC3)ccc2C(=O)N1C1CCC(=O)NC1=O] | rel-3-[7-methyl-5-oxo-2-(4-{[(1r,4r)-4- [(3,3-dimethyl-4-{[2-(3-{3-[(4-methyl- 4H-1,2,4-triazol-3-yl)methyl]oxetan-3- yl}phenyl)-3-oxo-7-(trifluoromethyl)- 2,3-dihydro-1H-isoindol-5- yl]methyl}piperazin-1- yl)methyl]cyclohexyl]oxy}piperidin-1- yl)-5H,6H,7H-pyrrolo[3,4-b]pyridin-6- yl]piperidine-2,6-dione | Scheme E |
| 247 | 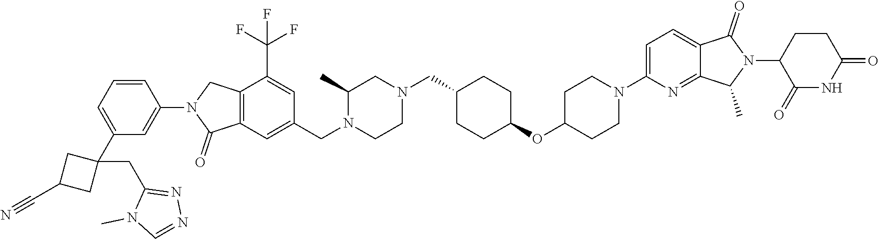 [SMILES: C[C@@H]1c2nc(N3CCC(O[C@H]4CC[C@H](CN5CCN(Cc6cc7c(c(C(F)(F)F)c6)CN(c6cccc(C8(Cc9nncn9C)CC(C#N)C8)c6)C7=O)[C@@H](C)C5)CC4)CC3)ccc2C(=O)N1C1CCC(=O)NC1=O] | (1r***,3r***)-3-[3-(6-{[(2S)-2-methyl- 4-{[(1r**,4r**)-4-({1-[(7R*)-6-(2,6- dioxopiperidin-3-yl)-7-methyl-5-oxo- 5H,6H,7H-pyrrolo[3,4-b]pyridin-2- yl]piperidin-4- yl}oxy)cyclohexyl]methyl}piperazin-1- yl]methyl}-1-oxo-4-(trifluoromethyl)- 2,3-dihydro-1H-isoindol-2-yl)phenyl]- 3-[(4-methyl-4H-1,2,4-triazol-3- yl)methyl]cyclobutane-1-carbonitrile | Scheme E |
| 248 | 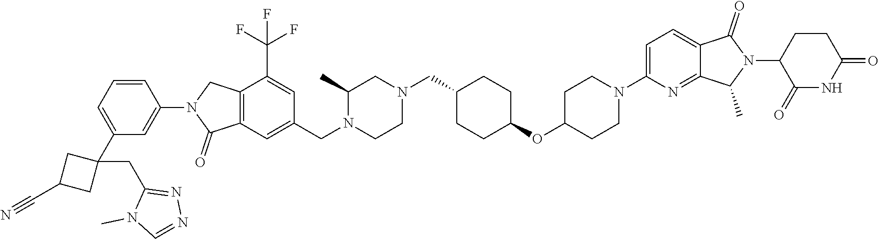 [SMILES: C[C@@H]1c2nc(N3CCC(O[C@H]4CC[C@H](CN5CCN(Cc6cc7c(c(C(F)(F)F)c6)CN(c6cccc(C8(Cc9nncn9C)CC(C#N)C8)c6)C7=O)[C@@H](C)C5)CC4)CC3)ccc2C(=O)N1C1CCC(=O)NC1=O] | (1r***,3r***)-3-[3-(6-{[(2S)-2-methyl- 4-{[(1r**,4r**)-4-({1-[(7R*)-6-(2,6- dioxopiperidin-3-yl)-7-methyl-5-oxo- 5H,6H,7H-pyrrolo[3,4-b]pyridin-2- yl]piperidin-4- yl}oxy)cyclohexyl]methyl}piperazin-1- yl]methyl}-1-oxo-4-(trifluoromethyl)- 2,3-dihydro-1H-isoindol-2-yl)phenyl]- 3-[(4-methyl-4H-1,2,4-triazol-3- yl)methyl]cyclobutane-1-carbonitrile | Scheme E |
| 249 | 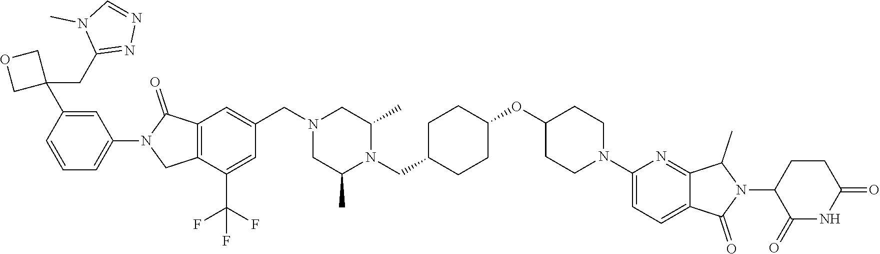 [SMILES: CC1c2nc(N3CCC(O[C@H]4CC[C@@H](CN5[C@@H](C)CN(Cc6cc7c(c(C(F)(F)F)c6)CN(c6cccc(C8(Cc9nncn9C)COC8)c6)C7=O)C[C@@H]5C)CC4)CC3)ccc2C(=O)N1C1CCC(=O)NC1=O] | 3-[7-methyl-5-oxo-2-(4-{[(1r*,4r*)-4- {[(2S,6S)-2,6-dimethyl-4-{[2-(3-{3- [(4-methyl-4H-1,2,4-triazol-3- yl)methyl]oxetan-3-yl}phenyl)-3-oxo- 7-(trifluoromethyl)-2,3-dihydro-1H- isoindol-5-yl]methyl}piperazin-1- yl]methyl}cyclohexyl]oxy}piperidin-1- yl)-5H,6H,7H-pyrrolo[3,4-b]pyridin-6- yl]piperidine-2,6-dione | Scheme E |
| 250 | 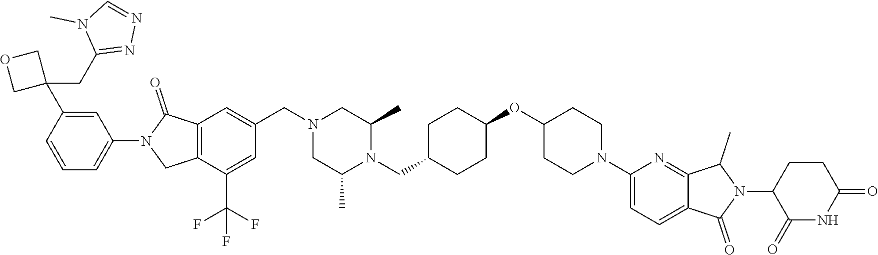 [SMILES: CC1c2nc(N3CCC(O[C@H]4CC[C@H](CN5[C@H](C)CN(Cc6cc7c(c(C(F)(F)F)c6)CN(c6cccc(C8(Cc9nncn9C)COC8)c6)C7=O)C[C@H]5C)CC4)CC3)ccc2C(=O)N1C1CCC(=O)NC1=O] | 3-[7-methyl-5-oxo-2-(4-{[(1r*,4r*)-4- {[(2R,6R)-2,6-dimethyl-4-{[2-(3-{3- [(4-methyl-4H-1,2,4-triazol-3- yl)methyl]oxetan-3-yl}phenyl)-3-oxo- 7-(trifluoromethyl)-2,3-dihydro-1H- isoindol-5-yl]methyl}piperazin-1- yl]methyl}cyclohexyl]oxy}piperidin-1- yl)-5H,6H,7H-pyrrolo[3,4-b]pyridin-6- yl]piperidine-2,6-dione | Scheme E |
| 251 | 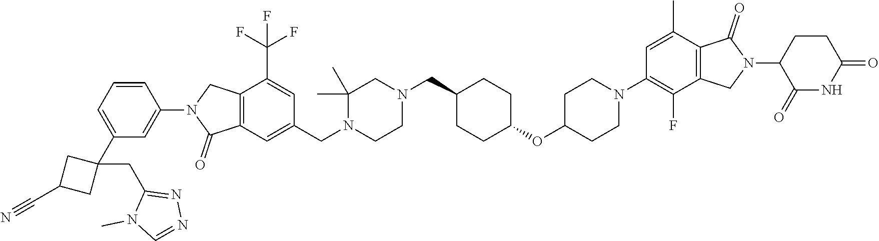 [SMILES: Cc1cc(N2CCC(O[C@H]3CC[C@H](CN4CCN(Cc5cc6c(c(C(F)(F)F)c5)CN(c5cccc(C7(Cc8nncn8C)CC(C#N)C7)c5)C6=O)C(C)(C)C4)CC3)CC2)c(F)c2c1C(=O)N(C1CCC(=O)NC1=O)C2] | (1r**,3r**)-3-(3-{6-[(2,2-dimethyl-4- {[(1r*,4r*)-4-({1-[2-(2,6- dioxopiperidin-3-yl)-4-fluoro-7- methyl-1-oxo-2,3-dihydro-1H-isoindol- 5-yl]piperidin-4- yl}oxy)cyclohexyl]methyl}piperazin-1- yl)methyl]-1-oxo-4-(trifluoromethyl)- 2,3-dihydro-1H-isoindol-2-yl}phenyl)- 3-[(4-methyl-4H-1,2,4-triazol-3- yl)methyl]cyclobutane-1-carbonitrile | Scheme E |
| 252 | 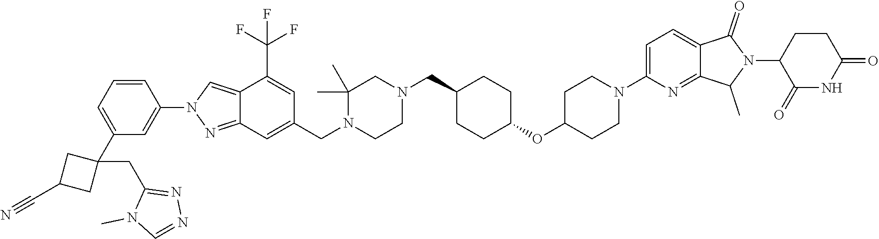 [SMILES: CC1c2nc(N3CCC(O[C@H]4CC[C@H](CN5CCN(Cc6cc(C(F)(F)F)c7cn(-c8cccc(C9(Cc%10nncn%10C)CC(C#N)C9)c8)nc7c6)C(C)(C)C5)CC4)CC3)ccc2C(=O)N1C1CCC(=O)NC1=O] | (1r**,3r**)-3-(3-{6-[(2,2-dimethyl-4- {[(1r*,4r*)-4-({1-[6-(2,6- dioxopiperidin-3-yl)-7-methyl-5-oxo- 5H,6H,7H-pyrrolo[3,4-b]pyridin-2- yl]piperidin-4- yl}oxy)cyclohexyl]methyl}piperazin-1- yl)methyl]-4-(trifluoromethyl)-2H- indazol-2-yl}phenyl)-3-[(4-methyl-4H- 1,2,4-triazol-3-yl)methyl]cyclobutane- 1-carbonitrile | Scheme E |
| 253 | 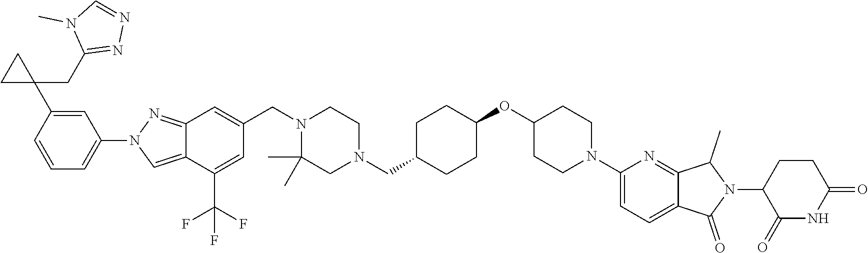 [SMILES: CC1c2nc(N3CCC(O[C@H]4CC[C@H](CN5CCN(Cc6cc(C(F)(F)F)c7cn(-c8cccc(C9(Cc%10nncn%10C)CC9)c8)nc7c6)C(C)(C)C5)CC4)CC3)ccc2C(=O)N1C1CCC(=O)NC1=O] | rel-3-[7-methyl-5-oxo-2-(4-{[(1r,4r)-4- [(3,3-dimethyl-4-{[2-(3-{1-[(4-methyl- 4H-1,2,4-triazol-3- yl)methyl]cyclopropyl}phenyl)-4- (trifluoromethyl)-2H-indazol-6- yl]methyl}piperazin-1- yl)methyl]cyclohexyl]oxy}piperidin-1- yl)-5H,6H,7H-pyrrolo[3,4-b]pyridin-6- yl]piperidine-2,6-dione | Scheme E |
| 254 | 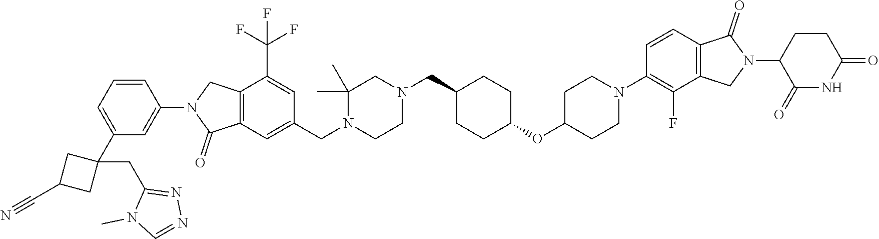 [SMILES: Cn1cnnc1CC1(c2cccc(N3Cc4c(cc(CN5CCN(C[C@H]6CC[C@H](OC7CCN(c8ccc9c(c8F)CN(C8CCC(=O)NC8=O)C9=O)CC7)CC6)CC5(C)C)cc4C(F)(F)F)C3=O)c2)CC(C#N)C1] | (1r**,3r**)-3-(3-{6-[(2,2-dimethyl-4- {[(1r*,4r*)-4-({1-[2-(2,6- dioxopiperidin-3-yl)-4-fluoro-1-oxo- 2,3-dihydro-1H-isoindol-5- yl]piperidin-4- yl}oxy)cyclohexyl]methyl}piperazin-1- yl)methyl]-1-oxo-4-(trifluoromethyl)- 2,3-dihydro-1H-isoindol-2-yl}phenyl)- 3-[(4-methyl-4H-1,2,4-triazol-3- yl)methyl]cyclobutane-1-carbonitrile | Scheme E |
| 255 | 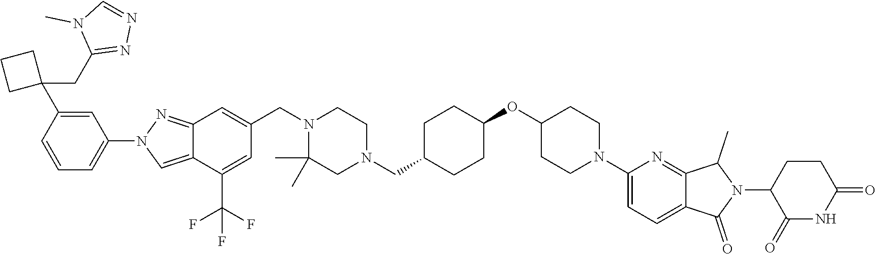 [SMILES: CC1c2nc(N3CCC(O[C@H]4CC[C@H](CN5CCN(Cc6cc(C(F)(F)F)c7cn(-c8cccc(C9(Cc%10nncn%10C)CCC9)c8)nc7c6)C(C)(C)C5)CC4)CC3)ccc2C(=O)N1C1CCC(=O)NC1=O] | rel-3-[7-methyl-5-oxo-2-(4-{[(1r,4r)-4- [(3,3-dimethyl-4-{[2-(3-{1-[(4-methyl- 4H-1,2,4-triazol-3- yl)methyl]cyclobutyl}phenyl)-4- (trifluoromethyl)-2H-indazol-6- yl]methyl}piperazin-1- yl)methyl]cyclohexyl]oxy}piperidin-1- yl)-5H,6H,7H-pyrrolo[3,4-b]pyridin-6- yl]piperidine-2,6-dione | Scheme E |
| 256 | 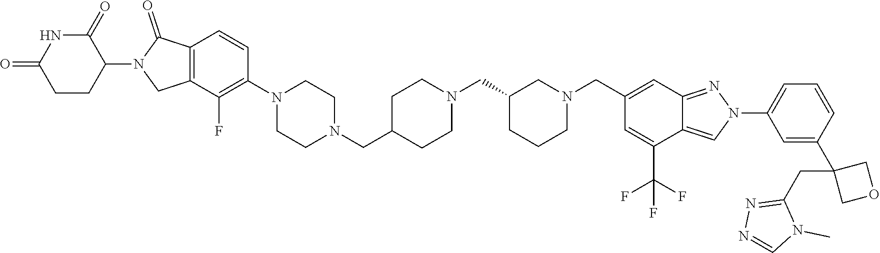 [SMILES: Cn1cnnc1CC1(c2cccc(-n3cc4c(C(F)(F)F)cc(CN5CCC[C@H](CN6CCC(CN7CCN(c8ccc9c(c8F)CN(C8CCC(=O)NC8=O)C9=O)CC7)CC6)C5)cc4n3)c2)COC1] | 3-(4-fluoro-5-{4-[(1-{[(3R)-1-{[2-(3- {3-[(4-methyl-4H-1,2,4-triazol-3- yl)methyl]oxetan-3-yl}phenyl)-4- (trifluoromethyl)-2H-indazol-6- yl]methyl}piperidin-3- yl]methyl}piperidin-4- yl)methyl]piperazin-1-yl}-1-oxo-2,3- dihydro-1H-isoindol-2-yl)piperidine- 2,6-dione | Scheme F |
| 257 | 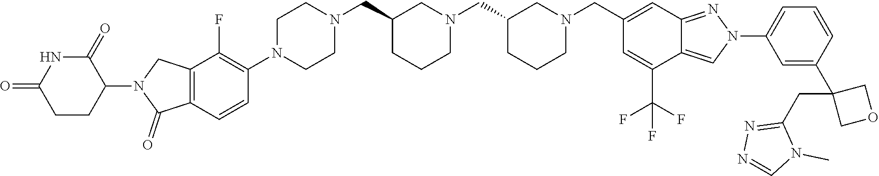 [SMILES: Cn1cnnc1CC1(c2cccc(-n3cc4c(C(F)(F)F)cc(CN5CCC[C@H](CN6CCC[C@@H](CN7CCN(c8ccc9c(c8F)CN(C8CCC(=O)NC8=O)C9=O)CC7)C6)C5)cc4n3)c2)COC1] | 3-[4-fluoro-5-(4-{[(3R)-1-{[(3R)-1- {[2-(3-{3-[(4-methyl-4H-1,2,4-triazol- 3-yl)methyl]oxetan-3-yl}phenyl)-4- (trifluoromethyl)-2H-indazol-6- yl]methyl}piperidin-3- yl]methyl}piperidin-3- yl]methyl}piperazin-1-yl)-1-oxo-2,3- dihydro-1H-isoindol-2-yl]piperidine- 2,6-dione | Scheme F |
| 258 | 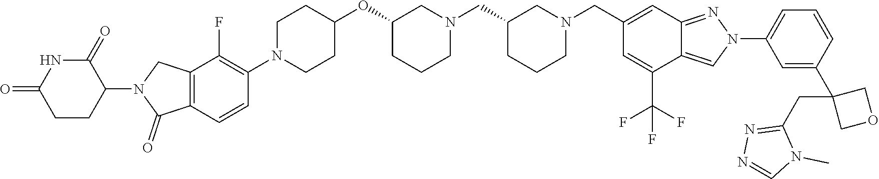 [SMILES: Cn1cnnc1CC1(c2cccc(-n3cc4c(C(F)(F)F)cc(CN5CCC[C@H](CN6CCC[C@H](OC7CCN(c8ccc9c(c8F)CN(C8CCC(=O)NC8=O)C9=O)CC7)C6)C5)cc4n3)c2)COC1] | 3-[4-fluoro-5-(4-{[(3S)-1-{[(3S)-1-{[2- (3-{3-[(4-methyl-4H-1,2,4-triazol-3- yl)methyl]oxetan-3-yl}phenyl)-4- (trifluoromethyl)-2H-indazol-6- yl]methyl}piperidin-3- yl]methyl}piperidin-3- yl]oxy}piperidin-1-yl)-1-oxo-2,3- dihydro-1H-isoindol-2-yl]piperidine- 2,6-dione | Scheme F |
| 259 | 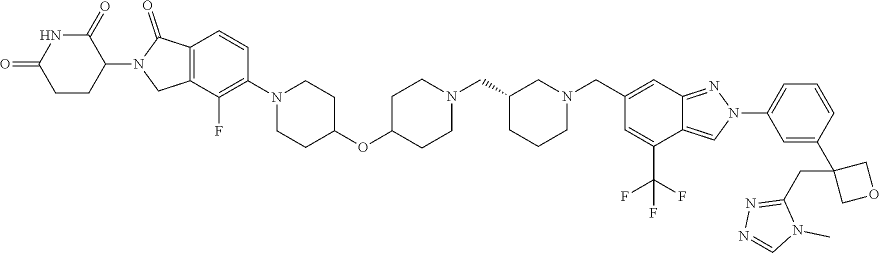 [SMILES: Cn1cnnc1CC1(c2cccc(-n3cc4c(C(F)(F)F)cc(CN5CCC[C@H](CN6CCC(OC7CCN(c8ccc9c(c8F)CN(C8CCC(=O)NC8=O)C9=O)CC7)CC6)C5)cc4n3)c2)COC1] | 3-(4-fluoro-5-{4-[(1-{[(3R)-1-{[2-(3- {3-[(4-methyl-4H-1,2,4-triazol-3- yl)methyl]oxetan-3-yl}phenyl)-4- (trifluoromethyl)-2H-indazol-6- yl]methyl}piperidin-3- yl]methyl}piperidin-4- yl)oxy]piperidin-1-yl}-1-oxo-2,3- dihydro-1H-isoindol-2-yl)piperidine- 2,6-dione | Scheme F |
| 260 | 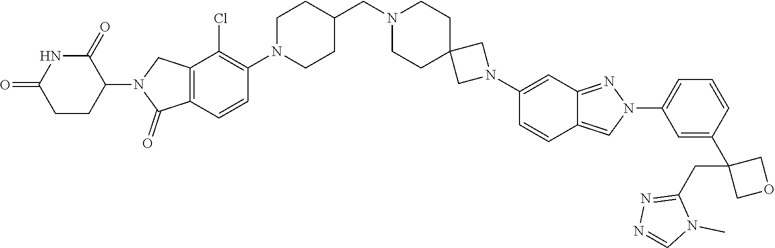 [SMILES: Cn1cnnc1CC1(c2cccc(-n3cc4ccc(N5CC6(CCN(CC7CCN(c8ccc9c(c8Cl)CN(C8CCC(=O)NC8=O)C9=O)CC7)CC6)C5)cc4n3)c2)COC1] | 3-{4-chloro-5-[4-({2-[2-(3-{3-[(4- methyl-4H-1,2,4-triazol-3- yl)methyl]oxetan-3-yl}phenyl)-2H- indazol-6-yl]-2,7-diazaspiro[3.5]nonan- 7-yl}methyl)piperidin-1-yl]-1-oxo-2,3- dihydro-1H-isoindol-2-yl}piperidine- 2,6-dione | Scheme G |
| 261 | 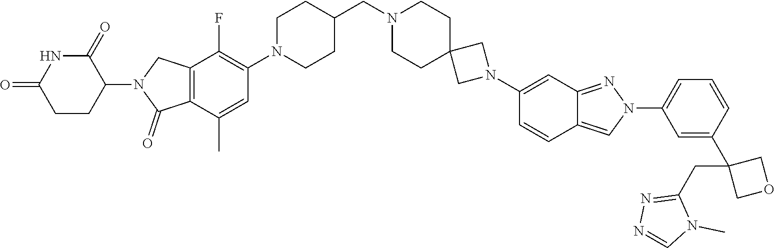 [SMILES: Cc1cc(N2CCC(CN3CCC4(CC3)CN(c3ccc5cn(-c6cccc(C7(Cc8nncn8C)COC7)c6)nc5c3)C4)CC2)c(F)c2c1C(=O)N(C1CCC(=O)NC1=O)C2] | 3-{4-fluoro-7-methyl-5-[4-({2-[2-(3- {3-[(4-methyl-4H-1,2,4-triazol-3- yl)methyl]oxetan-3-yl}phenyl)-2H- indazol-6-yl]-2,7-diazaspiro[3.5]nonan- 7-yl}methyl)piperidin-1-yl]-1-oxo-2,3- dihydro-1H-isoindol-2-yl}piperidine- 2,6-dione | Scheme G |
| 262 | 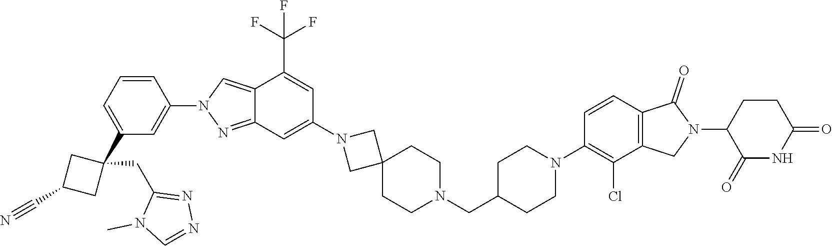 [SMILES: Cn1cnnc1C[C@]1(c2cccc(-n3cc4c(C(F)(F)F)cc(N5CC6(CCN(CC7CCN(c8ccc9c(c8Cl)CN(C8CCC(=O)NC8=O)C9=O)CC7)CC6)C5)cc4n3)c2)C[C@H](C#N)C1] | (1r,3r)-3-(3-{6-[7-({1-[4-chloro-2-(2,6- dioxopiperidin-3-yl)-1-oxo-2,3- dihydro-1H-isoindol-5-yl]piperidin-4- yl}methyl)-2,7-diazaspiro[3.5]nonan-2- yl]-4-(trifluoromethyl)-2H-indazol-2- yl}phenyl)-3-[(4-methyl-4H-1,2,4- triazol-3-yl)methyl]cyclobutane-1- carbonitrile | Scheme G |
| 263 | 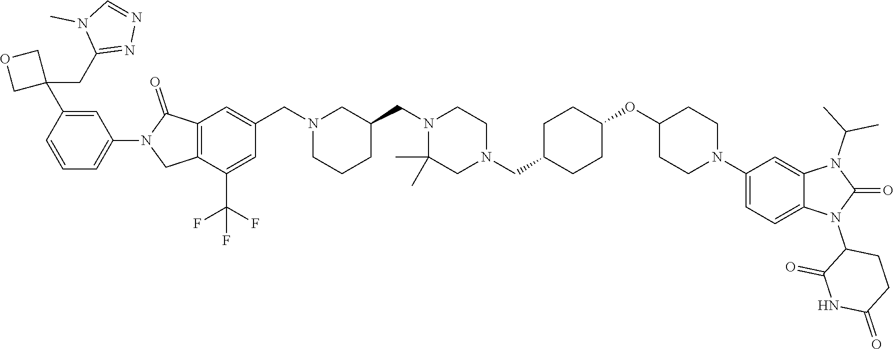 [SMILES: CC(C)n1c(=O)n(C2CCC(=O)NC2=O)c2ccc(N3CCC(O[C@H]4CC[C@@H](CN5CCN(C[C@H]6CCCN(Cc7cc8c(c(C(F)(F)F)c7)CN(c7cccc(C9(Cc%10nncn%10C)COC9)c7)C8=O)C6)C(C)(C)C5)CC4)CC3)cc21] | 3-[2-oxo-3-(propan-2-yl)-5-(4- {[(1r,4r)-4-[(3,3-dimethyl-4-{[(3S)-1- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}piperidin-3- yl]methyl}piperazin-1- yl)methyl]cyclohexyl]oxy}piperidin-1- yl)-2,3-dihydro-1H-1,3-benzodiazol-1- yl]piperidine-2,6-dione | Example 146 |
| 264 | 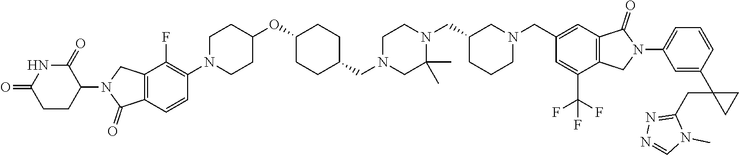 [SMILES: Cn1cnnc1CC1(c2cccc(N3Cc4c(cc(CN5CCC[C@H](CN6CCN(C[C@H]7CC[C@@H](OC8CCN(c9ccc%10c(c9F)CN(C9CCC(=O)NC9=O)C%10=O)CC8)CC7)CC6(C)C)C5)cc4C(F)(F)F)C3=O)c2)CC1] | 3-[4-fluoro-1-oxo-5-(4-{[(1r,4r)-4- [(3,3-dimethyl-4-{[(3S)-1-{[2-(3-{1- [(4-methyl-4H-1,2,4-triazol-3- yl)methyl]cyclopropyl}phenyl)-3-oxo- 7-(trifluoromethyl)-2,3-dihydro-1H- isoindol-5-yl]methyl}piperidin-3- yl]methyl}piperazin-1- yl)methyl]cyclohexyl]oxy}piperidin-1- yl)-2,3-dihydro-1H-isoindol-2- yl]piperidine-2,6-dione | Example 146 |
| 265 | 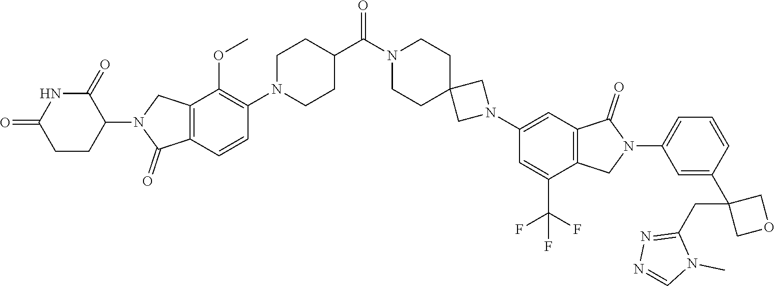 [SMILES: COc1c(N2CCC(C(=O)N3CCC4(CC3)CN(c3cc5c(c(C(F)(F)F)c3)CN(c3cccc(C6(Cc7nncn7C)COC6)c3)C5=O)C4)CC2)ccc2c1CN(C1CCC(=O)NC1=O)C2=O] | 3-[4-methoxy-5-(4-{2-[2-(3-{3-[(4- methyl-4H-1,2,4-triazol-3- yl)methyl]oxetan-3-yl}phenyl)-3-oxo- 7-(trifluoromethyl)-2,3-dihydro-1H- isoindol-5-yl]-2,7- diazaspiro[3.5]nonane-7- carbonyl}piperidin-1-yl)-1-oxo-2,3- dihydro-1H-isoindol-2-yl]piperidine- 2,6-dione | Example 146 |
| 266 | 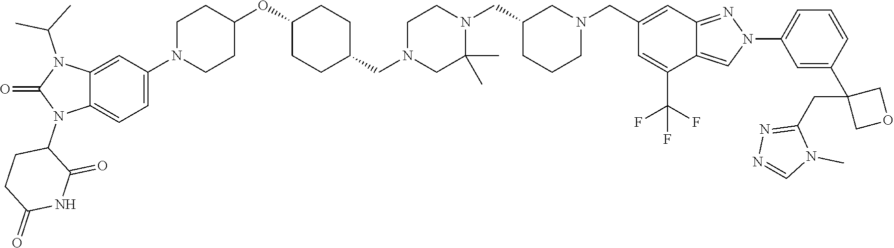 [SMILES: CC(C)n1c(=O)n(C2CCC(=O)NC2=O)c2ccc(N3CCC(O[C@H]4CC[C@@H](CN5CCN(C[C@H]6CCCN(Cc7cc(C(F)(F)F)c8cn(-c9cccc(C%10(Cc%11nncn%11C)COC%10)c9)nc8c7)C6)C(C)(C)C5)CC4)CC3)cc21] | 3-[2-oxo-3-(propan-2-yl)-5-(4- {[(1r,4r)-4-[(3,3-dimethyl-4-{[(3S)-1- {[2-(3-{3-[(4-methyl-4H-1,2,4-triazol- 3-yl)methyl]oxetan-3-yl}phenyl)-4- (trifluoromethyl)-2H-indazol-6- yl]methyl}piperidin-3- yl]methyl}piperazin-1- yl)methyl]cyclohexyl]oxy}piperidin-1- yl)-2,3-dihydro-1H-1,3-benzodiazol-1- yl]piperidine-2,6-dione | Example 148 |
| 267 | 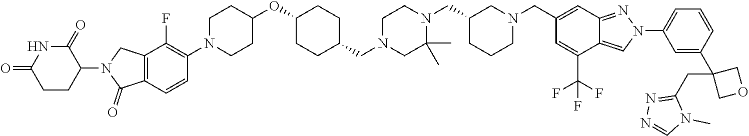 [SMILES: Cn1cnnc1CC1(c2cccc(-n3cc4c(C(F)(F)F)cc(CN5CCC[C@H](CN6CCN(C[C@H]7CC[C@@H](OC8CCN(c9ccc%10c(c9F)CN(C9CCC(=O)NC9=O)C%10=O)CC8)CC7)CC6(C)C)C5)cc4n3)c2)COC1] | 3-[4-fluoro-1-oxo-5-(4-{[(1r,4r)-4- [(3,3-dimethyl-4-{[(3S)-1-{[2-(3-{3- [(4-methyl-4H-1,2,4-triazol-3- yl)methyl]oxetan-3-yl}phenyl)-4- (trifluoromethyl)-2H-indazol-6- yl]methyl}piperidin-3- yl]methyl}piperazin-1- yl)methyl]cyclohexyl]oxy}piperidin-1- yl)-2,3-dihydro-1H-isoindol-2- yl]piperidine-2,6-dione | Example 148 |
| 268 | 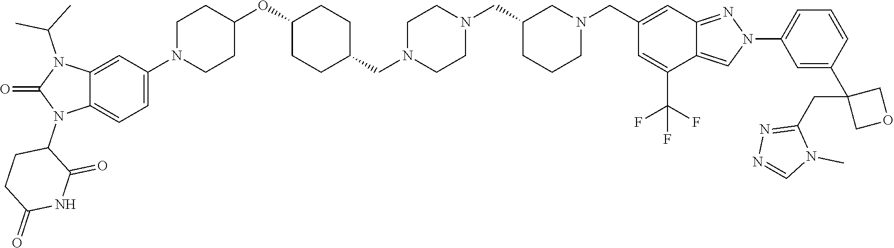 [SMILES: CC(C)n1c(=O)n(C2CCC(=O)NC2=O)c2ccc(N3CCC(O[C@H]4CC[C@@H](CN5CCN(C[C@H]6CCCN(Cc7cc(C(F)(F)F)c8cn(-c9cccc(C%10(Cc%11nncn%11C)COC%10)c9)nc8c7)C6)CC5)CC4)CC3)cc21] | 3-[2-oxo-3-(propan-2-yl)-5-(4- {[(1r,4r)-4-[(4-{[(3S)-1-{[2-(3-{3-[(4- methyl-4H-1,2,4-triazol-3- yl)methyl]oxetan-3-yl}phenyl)-4- (trifluoromethyl)-2H-indazol-6- yl]methyl}piperidin-3- yl]methyl}piperazin-1- yl)methyl]cyclohexyl]oxy}piperidin-1- yl)-2,3-dihydro-1H-1,3-benzodiazol-1- yl]piperidine-2,6-dione | Example 148 |
| 269 | 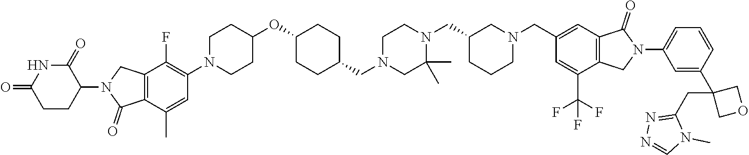 [SMILES: Cc1cc(N2CCC(O[C@H]3CC[C@@H](CN4CCN(C[C@H]5CCCN(Cc6cc7c(c(C(F)(F)F)c6)CN(c6cccc(C8(Cc9nncn9C)COC8)c6)C7=O)C5)C(C)(C)C4)CC3)CC2)c(F)c2c1C(=O)N(C1CCC(=O)NC1=O)C2] | 3-[4-fluoro-7-methyl-1-oxo-5-(4- {[(1r,4r)-4-[(3,3-dimethyl-4-{[(3S)-1- {[2-(3-{3-[(4-methyl-4H-1,2,4-triazol- 3-yl)methyl]oxetan-3-yl}phenyl)-3- oxo-7-(trifluoromethyl)-2,3-dihydro- 1H-isoindol-5-yl]methyl}piperidin-3- yl]methyl}piperazin-1- yl)methyl]cyclohexyl]oxy}piperidin-1- yl)-2,3-dihydro-1H-isoindol-2- yl]piperidine-2,6-dione | Example 148 |
| 270 | 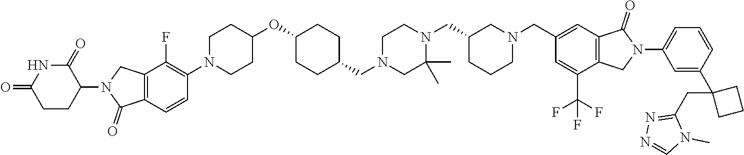 [SMILES: Cn1cnnc1CC1(c2cccc(N3Cc4c(cc(CN5CCC[C@H](CN6CCN(C[C@H]7CC[C@@H](OC8CCN(c9ccc%10c(c9F)CN(C9CCC(=O)NC9=O)C%10=O)CC8)CC7)CC6(C)C)C5)cc4C(F)(F)F)C3=O)c2)CCC1] | 3-[4-fluoro-1-oxo-5-(4-{[(1r,4r)-4- [(3,3-dimethyl-4-{[(3S)-1-{[2-(3-{1- [(4-methyl-4H-1,2,4-triazol-3- yl)methyl]cyclobutyl}phenyl)-3-oxo-7- (trifluoromethyl)-2,3-dihydro-1H- isoindol-5-yl]methyl}piperidin-3- yl]methyl}piperazin-1- yl)methyl]cyclohexyl]oxy}piperidin-1- yl)-2,3-dihydro-1H-isoindol-2- yl]piperidine-2,6-dione | Example 148 |
| 271 | 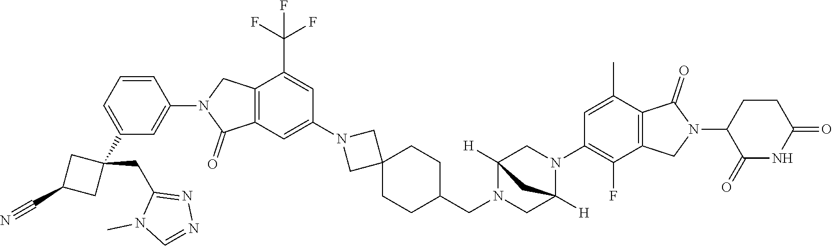 [SMILES: Cc1cc(N2C[C@@H]3C[C@H]2CN3CC2CCC3(CC2)CN(c2cc4c(c(C(F)(F)F)c2)CN(c2cccc([C@]5(Cc6nncn6C)C[C@@H](C#N)C5)c2)C4=O)C3)c(F)c2c1C(=O)N(C1CCC(=O)NC1=O)C2] | (1r,3r)-3-{3-[6-(7-{[(1S,4S)-5-[2-(2,6- dioxopiperidin-3-yl)-4-fluoro-7- methyl-1-oxo-2,3-dihydro-1H-isoindol- 5-yl]-2,5-diazabicyclo[2.2.1]heptan-2- yl]methyl}-2-azaspiro[3.5]nonan-2-yl)- 1-oxo-4-(trifluoromethyl)-2,3-dihydro- 1H-isoindol-2-yl]phenyl}-3-[(4- methyl-4H-1,2,4-triazol-3- yl)methyl]cyclobutane-1-carbonitrile | Example 150 |
| 272 | 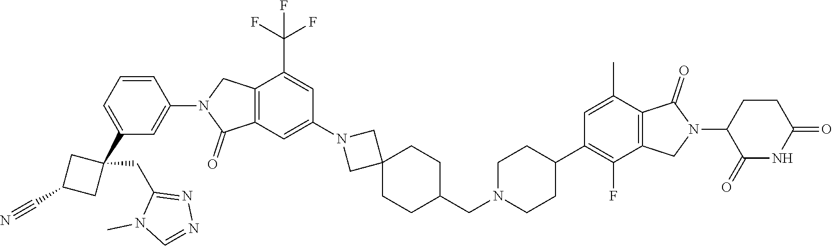 [SMILES: Cc1cc(C2CCN(CC3CCC4(CC3)CN(c3cc5c(c(C(F)(F)F)c3)CN(c3cccc([C@]6(Cc7nncn7C)C[C@@H](C#N)C6)c3)C5=O)C4)CC2)c(F)c2c1C(=O)N(C1CCC(=O)NC1=O)C2] | (1r,3r)-3-(3-{6-[7-({4-[2-(2,6- dioxopiperidin-3-yl)-4-fluoro-7- methyl-1-oxo-2,3-dihydro-1H-isoindol- 5-yl]piperidin-1-yl}methyl)-2- azaspiro[3.5]nonan-2-yl]-1-oxo-4- (trifluoromethyl)-2,3-dihydro-1H- isoindol-2-yl}phenyl)-3-[(4-methyl- 4H-1,2,4-triazol-3- yl)methyl]cyclobutane-1-carbonitrile | Example 150 |
| 273 | 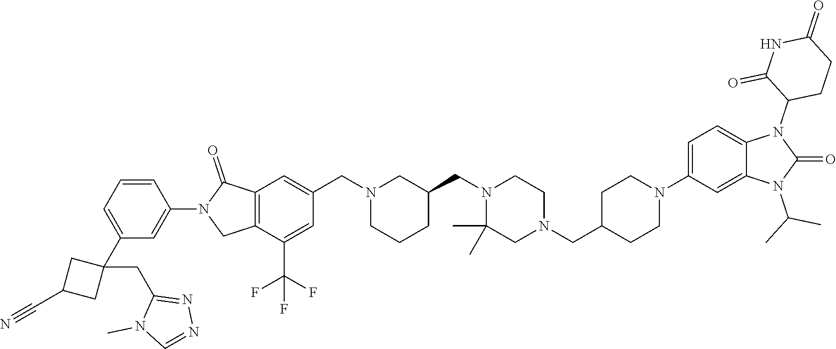 [SMILES: CC(C)n1c(=O)n(C2CCC(=O)NC2=O)c2ccc(N3CCC(CN4CCN(C[C@H]5CCCN(Cc6cc7c(c(C(F)(F)F)c6)CN(c6cccc(C8(Cc9nncn9C)CC(C#N)C8)c6)C7=O)C5)C(C)(C)C4)CC3)cc21] | (1r*,3r*)-3-[3-(6-{[(3S)-3-{[4-({1-[1- (2,6-dioxopiperidin-3-yl)-2-oxo-3- (propan-2-yl)-2,3-dihydro-1H-1,3- benzodiazol-5-yl]piperidin-4- yl}methyl)-2,2-dimethylpiperazin-1- yl]methyl}piperidin-1-yl]methyl}-1- oxo-4-(trifluoromethyl)-2,3-dihydro- 1H-isoindol-2-yl)phenyl]-3-[(4-methyl- 4H-1,2,4-triazol-3- yl)methyl]cyclobutane-1-carbonitrile | Example 153 |
| 274 | 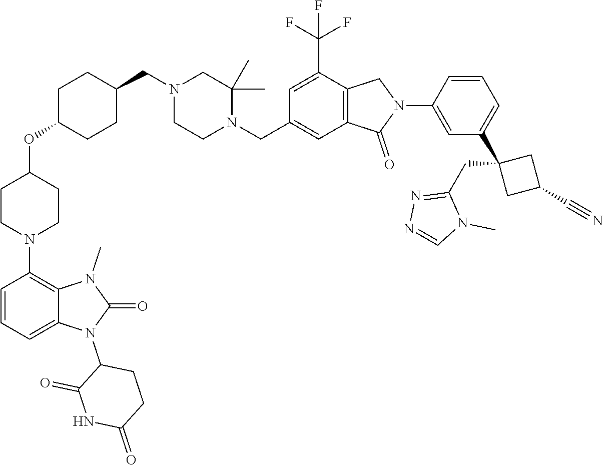 [SMILES: Cn1cnnc1C[C@]1(c2cccc(N3Cc4c(cc(CN5CCN(C[C@H]6CC[C@H](OC7CCN(c8cccc9c8n(C)c(=O)n9C8CCC(=O)NC8=O)CC7)CC6)CC5(C)C)cc4C(F)(F)F)C3=O)c2)C[C@H](C#N)C1] | (1r,3r)-3-(3-{6-[(2,2-dimethyl-4- {[(1r,4r)-4-({1-[1-(2,6-dioxopiperidin- 3-yl)-3-methyl-2-oxo-2,3-dihydro-1H- 1,3-benzodiazol-4-yl]piperidin-4- yl}oxy)cyclohexyl]methyl}piperazin-1- yl)methyl]-1-oxo-4-(trifluoromethyl)- 2,3-dihydro-1H-isoindol-2-yl}phenyl)- 3-[(4-methyl-4H-1,2,4-triazol-3- yl)methyl]cyclobutane-1-carbonitrile | Scheme D |
| 275 | 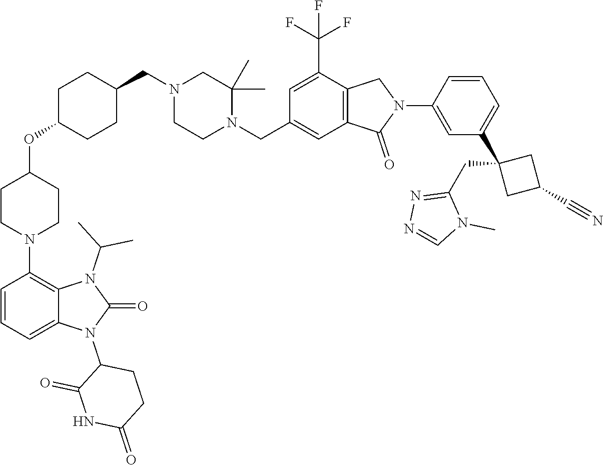 [SMILES: CC(C)n1c(=O)n(C2CCC(=O)NC2=O)c2cccc(N3CCC(O[C@H]4CC[C@H](CN5CCN(Cc6cc7c(c(C(F)(F)F)c6)CN(c6cccc([C@]8(Cc9nncn9C)C[C@@H](C#N)C8)c6)C7=O)C(C)(C)C5)CC4)CC3)c21] | (1r,3r)-3-(3-{6-[(2,2-dimethyl-4- {[(1r,4r)-4-({1-[1-(2,6-dioxopiperidin- 3-yl)-2-oxo-3-(propan-2-yl)-2,3- dihydro-1H-1,3-benzodiazol-4- yl]piperidin-4- yl}oxy)cyclohexyl]methyl}piperazin-1- yl)methyl]-1-oxo-4-(trifluoromethyl)- 2,3-dihydro-1H-isoindol-2-yl}phenyl)- 3-[(4-methyl-4H-1,2,4-triazol-3- yl)methyl]cyclobutane-1-carbonitrile | Scheme D |
| 284 | 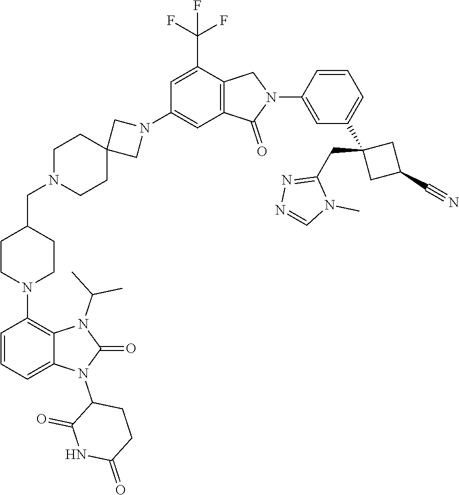 [SMILES: CC(C)n1c(=O)n(C2CCC(=O)NC2=O)c2cccc(N3CCC(CN4CCC5(CC4)CN(c4cc6c(c(C(F)(F)F)c4)CN(c4cccc([C@]7(Cc8nncn8C)C[C@@H](C#N)C7)c4)C6=O)C5)CC3)c21] | (1r,3r)-3-(3-{6-[7-({1-[1-(2,6- dioxopiperidin-3-yl)-2-oxo-3- (propan-2-yl)-2,3-dihydro-1H-1,3- benzodiazol-4-yl]piperidin-4- yl}methyl)-2,7-diazaspiro[3.5]nonan- 2-yl]-1-oxo-4-(trifluoromethyl)-2,3- dihydro-1H-isoindol-2-yl}phenyl)-3- [(4-methyl-4H-1,2,4-triazol-3- yl)methyl]cyclobutane-1-carbonitrile | Scheme B |
| 285 | 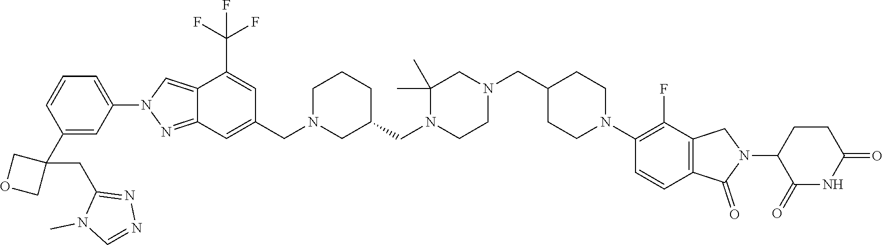 [SMILES: Cn1cnnc1CC1(c2cccc(-n3cc4c(C(F)(F)F)cc(CN5CCC[C@H](CN6CCN(CC7CCN(c8ccc9c(c8F)CN(C8CCC(=O)NC8=O)C9=O)CC7)CC6(C)C)C5)cc4n3)c2)COC1] | 3-(5-{4-[(3,3-dimethyl-4-{[(3S)-1- {[2-(3-{3-[(4-methyl-4H-1,2,4- triazol-3-yl)methyl]oxetan-3- yl}phenyl)-4-(trifluoromethyl)-2H- indazol-6-yl]methyl}piperidin-3- yl]methyl}piperazin-1- yl)methyl]piperidin-1-yl}-4-fluoro-1- oxo-2,3-dihydro-1H-isoindol-2- yl)piperidine-2,6-dione | Scheme D |
| 286 | 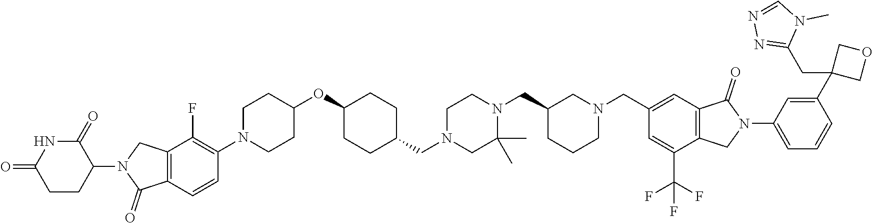 [SMILES: Cn1cnnc1CC1(c2cccc(N3Cc4c(cc(CN5CCC[C@@H](CN6CCN(C[C@H]7CC[C@H](OC8CCN(c9ccc%10c(c9F)CN(C9CCC(=O)NC9=O)C%10=O)CC8)CC7)CC6(C)C)C5)cc4C(F)(F)F)C3=O)c2)COC1] | 3-[4-fluoro-1-oxo-5-(4-{[(1r,4r)-4- [(3,3-dimethyl-4-{[(3R)-1-{[2-(3-{3- [(4-methyl-4H-1,2,4-triazol-3- yl)methyl]oxetan-3-yl}phenyl)-3- oxo-7-(trifluoromethyl)-2,3-dihydro- 1H-isoindol-5-yl]methyl}piperidin-3- yl]methyl}piperazin-1- yl)methyl]cyclohexyl]oxy}piperidin- 1-yl)-2,3-dihydro-1H-isoindol-2- yl]piperidine-2,6-dione | Scheme D |
| 287 | 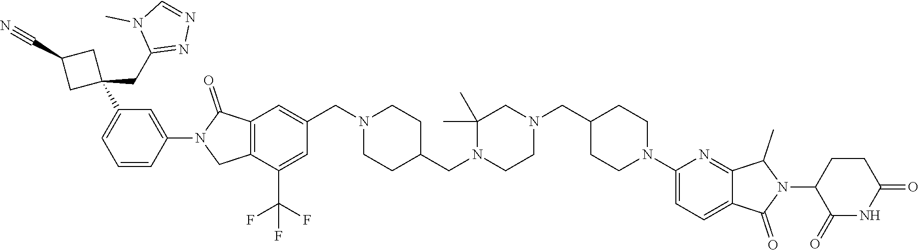 [SMILES: CC1c2nc(N3CCC(CN4CCN(CC5CCN(Cc6cc7c(c(C(F)(F)F)c6)CN(c6cccc([C@]8(Cc9nncn9C)C[C@@H](C#N)C8)c6)C7=O)CC5)C(C)(C)C4)CC3)ccc2C(=O)N1C1CCC(=O)NC1=O] | (1r,3r)-3-(3-{6-[(4-{[4-({1-[6-(2,6- dioxopiperidin-3-yl)-7-methyl-5-oxo- 5H,6H,7H-pyrrolo[3,4-b]pyridin-2- yl]piperidin-4-yl}methyl)-2,2- dimethylpiperazin-1- yl]methyl}piperidin-1-yl)methyl]-1- oxo-4-(trifluoromethyl)-2,3-dihydro- 1H-isoindol-2-yl}phenyl)-3-[(4- methyl-4H-1,2,4-triazol-3- yl)methyl]cyclobutane-1-carbonitrile | Scheme D |
| 288 | 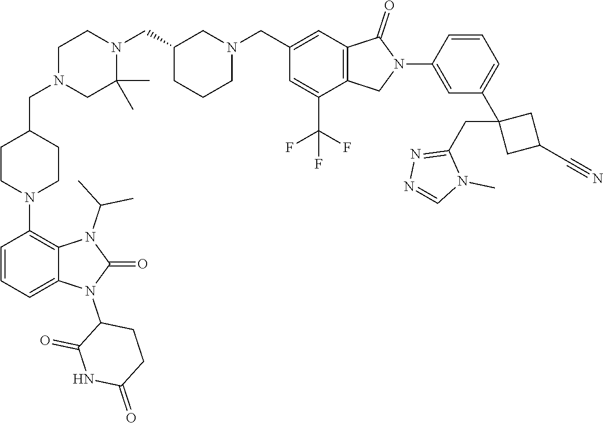 [SMILES: CC(C)n1c(=O)n(C2CCC(=O)NC2=O)c2cccc(N3CCC(CN4CCN(C[C@H]5CCCN(Cc6cc7c(c(C(F)(F)F)c6)CN(c6cccc(C8(Cc9nncn9C)CC(C#N)C8)c6)C7=O)C5)C(C)(C)C4)CC3)c21] | (1r*,3r*)-3-[3-(6-{[(3S)-3-{[4-({1- [1-(2,6-dioxopiperidin-3-yl)-2-oxo-3- (propan-2-yl)-2,3-dihydro-1H-1,3- benzodiazol-4-yl]piperidin-4- yl}methyl)-2,2-dimethylpiperazin-1- yl]methyl}piperidin-1-yl]methyl}-1- oxo-4-(trifluoromethyl)-2,3-dihydro- 1H-isoindol-2-yl)phenyl]-3-[(4- methyl-4H-1,2,4-triazol-3- yl)methyl]cyclobutane-1-carbonitrile | Scheme D |
| 289 | 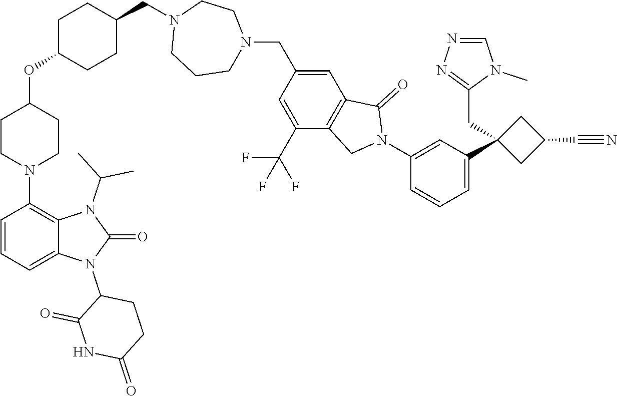 [SMILES: CC(C)n1c(=O)n(C2CCC(=O)NC2=O)c2cccc(N3CCC(O[C@H]4CC[C@H](CN5CCCN(Cc6cc7c(c(C(F)(F)F)c6)CN(c6cccc([C@]8(Cc9nncn9C)C[C@@H](C#N)C8)c6)C7=O)CC5)CC4)CC3)c21] | (1r,3r)-3-[(4-methyl-4H-1,2,4-triazol- 3-yl)methyl]-3-(3-{1-oxo-6-[(4- {[(1r,4r)-4-({1-[1-(2,6- dioxopiperidin-3-yl)-2-oxo-3- (propan-2-yl)-2,3-dihydro-1H-1,3- benzodiazol-4-yl]piperidin-4- yl}oxy)cyclohexyl]methyl}-1,4- diazepan-1-yl)methyl]-4- (trifluoromethyl)-2,3-dihydro-1H- isoindol-2-yl}phenyl)cyclobutane-1- carbonitrile | Scheme D |
| 290 | 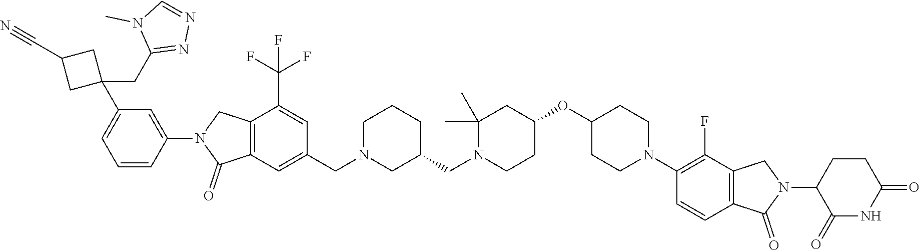 [SMILES: Cn1cnnc1CC1(c2cccc(N3Cc4c(cc(CN5CCC[C@H](CN6CC[C@@H](OC7CCN(c8ccc9c(c8F)CN(C8CCC(=O)NC8=O)C9=O)CC7)CC6(C)C)C5)cc4C(F)(F)F)C3=O)c2)CC(C#N)C1] | (1r**,3r**)-3-[3-(6-{[(3S)-3- {[(4R*)-4-({1-[2-(2,6- dioxopiperidin-3-yl)-4-fluoro-1-oxo- 2,3-dihydro-1H-isoindol-5- yl]piperidin-4-yl}oxy)-2,2- dimethylpiperidin-1- yl]methyl}piperidin-1-yl]methyl}-1- oxo-4-(trifluoromethyl)-2,3-dihydro- 1H-isoindol-2-yl)phenyl]-3-[(4- methyl-4H-1,2,4-triazol-3- yl)methyl]cyclobutane-1-carbonitrile | Scheme E |
| 291 | 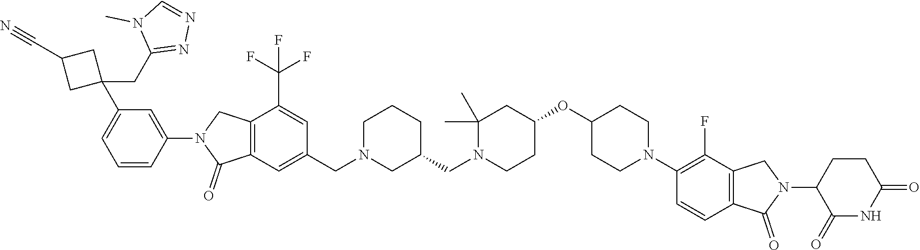 [SMILES: Cn1cnnc1CC1(c2cccc(N3Cc4c(cc(CN5CCC[C@H](CN6CC[C@@H](OC7CCN(c8ccc9c(c8F)CN(C8CCC(=O)NC8=O)C9=O)CC7)CC6(C)C)C5)cc4C(F)(F)F)C3=O)c2)CC(C#N)C1] | (1r**,3r**)-3-[3-(6-{[(3S)-3- {[(4R*)-4-({1-[2-(2,6- dioxopiperidin-3-yl)-4-fluoro-1-oxo- 2,3-dihydro-1H-isoindol-5- yl]piperidin-4-yl}oxy)-2,2- dimethylpiperidin-1- yl]methyl}piperidin-1-yl]methyl}-1- oxo-4-(trifluoromethyl)-2,3-dihydro- 1H-isoindol-2-yl)phenyl]-3-[(4- methyl-4H-1,2,4-triazol-3- yl)methyl]cyclobutane-1-carbonitrile | Scheme E |
| 292 | 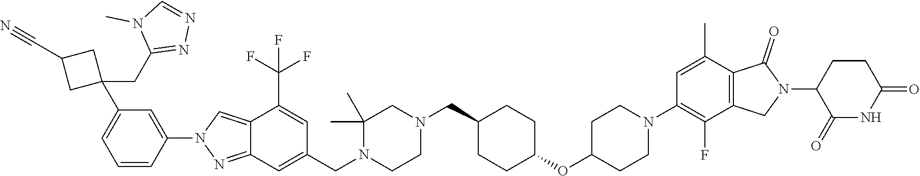 [SMILES: Cc1cc(N2CCC(O[C@H]3CC[C@H](CN4CCN(Cc5cc(C(F)(F)F)c6cn(-c7cccc(C8(Cc9nncn9C)CC(C#N)C8)c7)nc6c5)C(C)(C)C4)CC3)CC2)c(F)c2c1C(=O)N(C1CCC(=O)NC1=O)C2] | (1r**,3r**)-3-(3-{6-[(2,2-dimethyl- 4-{[(1r*,4r*)-4-({1-[2-(2,6- dioxopiperidin-3-yl)-4-fluoro-7- methyl-1-oxo-2,3-dihydro-1H- isoindol-5-yl]piperidin-4- yl}oxy)cyclohexyl]methyl}piperazin- 1-yl)methyl]-4-(trifluoromethyl)-2H- indazol-2-yl}phenyl)-3-[(4-methyl- 4H-1,2,4-triazol-3- yl)methyl]cyclobutane-1-carbonitrile | Scheme E |
| 293 | 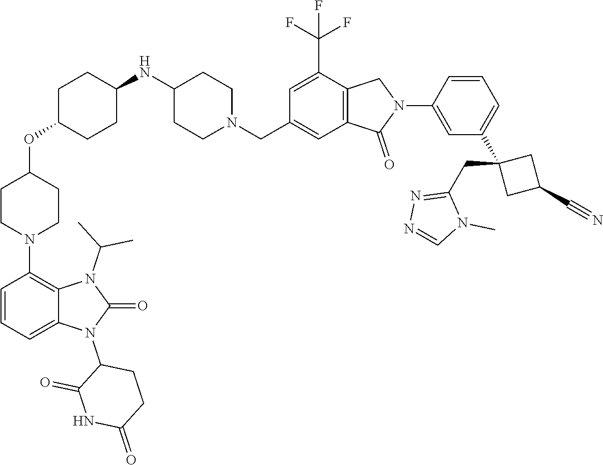 [SMILES: CC(C)n1c(=O)n(C2CCC(=O)NC2=O)c2cccc(N3CCC(O[C@H]4CC[C@H](NC5CCN(Cc6cc7c(c(C(F)(F)F)c6)CN(c6cccc([C@]8(Cc9nncn9C)C[C@@H](C#N)C8)c6)C7=O)CC5)CC4)CC3)c21] | (1r,3r)-3-[(4-methyl-4H-1,2,4-triazol- 3-yl)methyl]-3-(3-{1-oxo-6-[(4- {[(1r,4r)-4-({1-[1-(2,6- dioxopiperidin-3-yl)-2-oxo-3- (propan-2-yl)-2,3-dihydro-1H-1,3- benzodiazol-4-yl]piperidin-4- yl}oxy)cyclohexyl]amino}piperidin- 1-yl)methyl]-4-(trifluoromethyl)-2,3- dihydro-1H-isoindol-2- yl}phenyl)cyclobutane-1-carbonitrile | Example 276 |
| 294 | 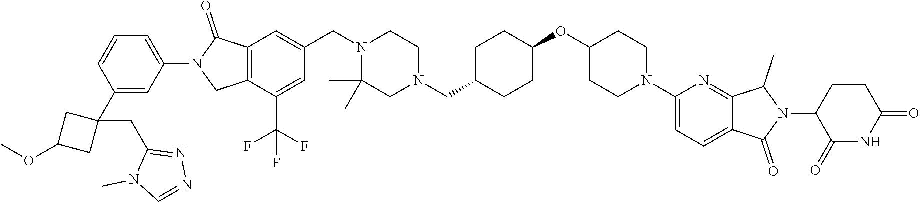 [SMILES: COC1CC(Cc2nncn2C)(c2cccc(N3Cc4c(cc(CN5CCN(C[C@H]6CC[C@H](OC7CCN(c8ccc9c(n8)C(C)N(C8CCC(=O)NC8=O)C9=O)CC7)CC6)CC5(C)C)cc4C(F)(F)F)C3=O)c2)C1] | 3-[7-methyl-5-oxo-2-(4-{[(1r,4r)-4- [(4-{[2-(3-{3-methoxy-1-[(4-methyl- 4H-1,2,4-triazol-3- yl)methyl]cyclobutyl}phenyl)-3-oxo- 7-(trifluoromethyl)-2,3-dihydro-1H- isoindol-5-yl]methyl}-3,3- dimethylpiperazin-1- yl)methyl]cyclohexyl]oxy}piperidin- 1-yl)-5H,6H,7H-pyrrolo[3,4- b]pyridin-6-yl]piperidine-2,6-dione | Example 279 |
| 295 | 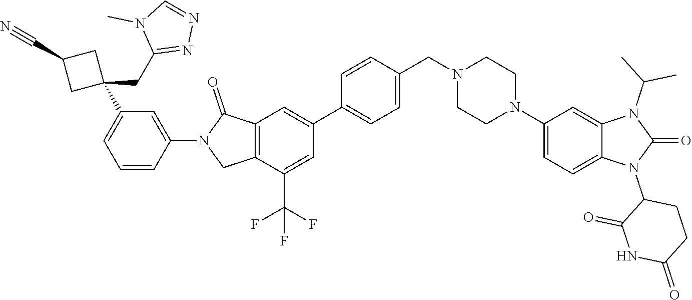 [SMILES: CC(C)n1c(=O)n(C2CCC(=O)NC2=O)c2ccc(N3CCN(Cc4ccc(-c5cc6c(c(C(F)(F)F)c5)CN(c5cccc([C@]7(Cc8nncn8C)C[C@@H](C#N)C7)c5)C6=O)cc4)CC3)cc21] | (1r,3r)-3-(3-{6-[4-({4-[1-(2,6- dioxopiperidin-3-yl)-2-oxo-3- (propan-2-yl)-2,3-dihydro-1H-1,3- benzodiazol-5-yl]piperazin-1- yl}methyl)phenyl]-1-oxo-4- (trifluoromethyl)-2,3-dihydro-1H- isoindol-2-yl}phenyl)-3-[(4-methyl- 4H-1,2,4-triazol-3- yl)methyl]cyclobutane-1-carbonitrile | Example 282 |
| 296 | 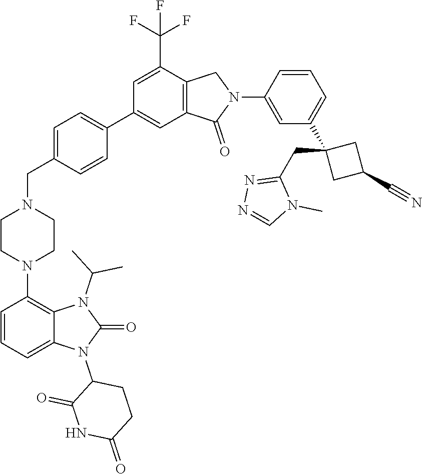 [SMILES: CC(C)n1c(=O)n(C2CCC(=O)NC2=O)c2cccc(N3CCN(Cc4ccc(-c5cc6c(c(C(F)(F)F)c5)CN(c5cccc([C@]7(Cc8nncn8C)C[C@@H](C#N)C7)c5)C6=O)cc4)CC3)c21] | (1r,3r)-3-(3-{6-[4-({4-[1-(2,6- dioxopiperidin-3-yl)-2-oxo-3- (propan-2-yl)-2,3-dihydro-1H-1,3- benzodiazol-4-yl]piperazin-1- yl}methyl)phenyl]-1-oxo-4- (trifluoromethyl)-2,3-dihydro-1H- isoindol-2-yl}phenyl)-3-[(4-methyl- 4H-1,2,4-triazol-3- yl)methyl]cyclobutane-1-carbonitrile | Example 292 |
| 297 | 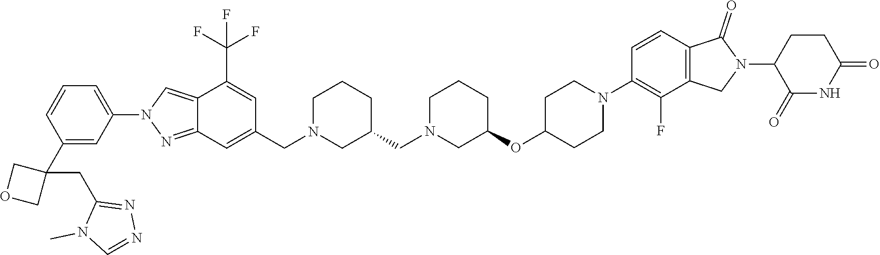 [SMILES: Cn1cnnc1CC1(c2cccc(-n3cc4c(C(F)(F)F)cc(CN5CCC[C@H](CN6CCC[C@@H](OC7CCN(c8ccc9c(c8F)CN(C8CCC(=O)NC8=O)C9=O)CC7)C6)C5)cc4n3)c2)COC1] | 3-[4-fluoro-5-(4-{[(3R)-1-{[(3S)-1- {[2-(3-{3-[(4-methyl-4H-1,2,4- triazol-3-yl)methyl]oxetan-3- yl}phenyl)-4-(trifluoromethyl)-2H- indazol-6-yl]methyl}piperidin-3- yl]methyl}piperidin-3- yl]oxy}piperidin-1-yl)-1-oxo-2,3- dihydro-1H-isoindol-2-yl]piperidine- 2,6-dione | Scheme F |
| 298 | 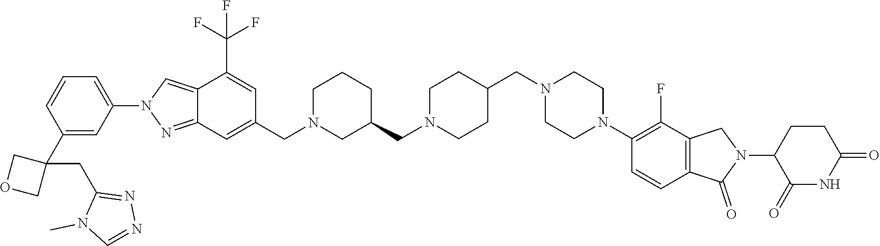 [SMILES: Cn1cnnc1CC1(c2cccc(-n3cc4c(C(F)(F)F)cc(CN5CCC[C@@H](CN6CCC(CN7CCN(c8ccc9c(c8F)CN(C8CCC(=O)NC8=O)C9=O)CC7)CC6)C5)cc4n3)c2)COC1] | 3-(4-fluoro-5-{4-[(1-{[(3S)-1-{[2-(3- {3-[(4-methyl-4H-1,2,4-triazol-3- yl)methyl]oxetan-3-yl}phenyl)-4- (trifluoromethyl)-2H-indazol-6- yl]methyl}piperidin-3- yl]methyl}piperidin-4- yl)methyl]piperazin-1-yl}-1-oxo-2,3- dihydro-1H-isoindol-2-yl)piperidine- 2,6-dione | Scheme F |
| 299 | 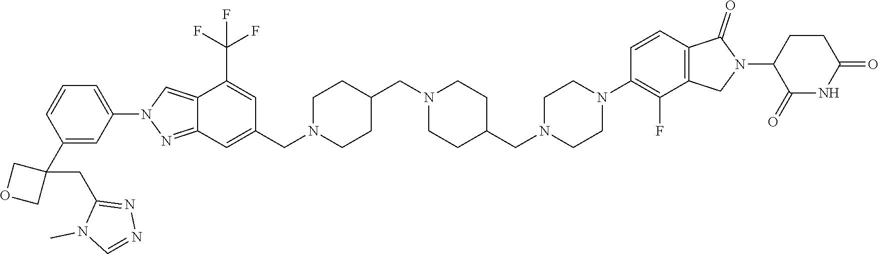 [SMILES: Cn1cnnc1CC1(c2cccc(-n3cc4c(C(F)(F)F)cc(CN5CCC(CN6CCC(CN7CCN(c8ccc9c(c8F)CN(C8CCC(=O)NC8=O)C9=O)CC7)CC6)CC5)cc4n3)c2)COC1] | 3-{4-fluoro-5-[4-({1-[(1-{[2-(3-{3- [(4-methyl-4H-1,2,4-triazol-3- yl)methyl]oxetan-3-yl}phenyl)-4- (trifluoromethyl)-2H-indazol-6- yl]methyl}piperidin-4- yl)methyl]piperidin-4- yl}methyl)piperazin-1-yl]-1-oxo-2,3- dihydro-1H-isoindol-2-yl}piperidine- 2,6-dione | Scheme F |
| 300 | 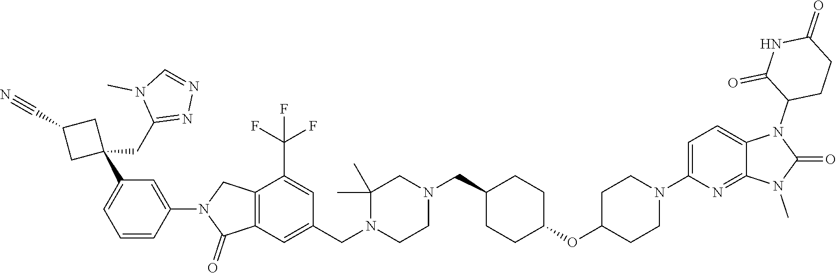 [SMILES: Cn1cnnc1C[C@]1(c2cccc(N3Cc4c(cc(CN5CCN(C[C@H]6CC[C@H](OC7CCN(c8ccc9c(n8)n(C)c(=O)n9C8CCC(=O)NC8=O)CC7)CC6)CC5(C)C)cc4C(F)(F)F)C3=O)c2)C[C@H](C#N)C1] | (1r,3r)-3-(3-{6-[(2,2-dimethyl-4- {[(1r,4r)-4-({1-[1-(2,6- dioxopiperidin-3-yl)-3-methyl-2-oxo- 1H,2H,3H-imidazo[4,5-b]pyridin-5- yl]piperidin-4- yl}oxy)cyclohexyl]methyl}piperazin- 1-yl)methyl]-1-oxo-4- (trifluoromethyl)-2,3-dihydro-1H- isoindol-2-yl}phenyl)-3-[(4-methyl- 4H-1,2,4-triazol-3- yl)methyl]cyclobutane-1-carbonitrile | Scheme H |
| 301 | 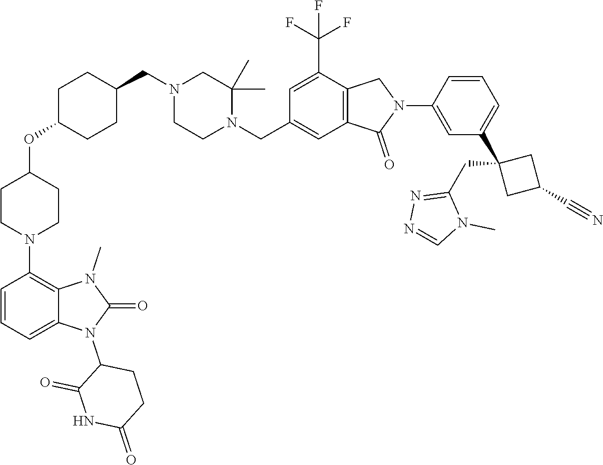 [SMILES: Cn1cnnc1C[C@]1(c2cccc(N3Cc4c(cc(CN5CCN(C[C@H]6CC[C@H](OC7CCN(c8cccc9c8n(C)c(=O)n9C8CCC(=O)NC8=O)CC7)CC6)CC5(C)C)cc4C(F)(F)F)C3=O)c2)C[C@H](C#N)C1] | (1r,3r)-3-(3-{6-[(2,2-dimethyl-4- {[(1r,4r)-4-({1-[1-(2,6- dioxopiperidin-3-yl)-3-methyl-2-oxo- 2,3-dihydro-1H-1,3-benzodiazol-4- yl]piperidin-4- yl}oxy)cyclohexyl]methyl}piperazin- 1-yl)methyl]-1-oxo-4- (trifluoromethyl)-2,3-dihydro-1H- isoindol-2-yl}phenyl)-3-[(4-methyl- 4H-1,2,4-triazol-3- yl)methyl]cyclobutane-1-carbonitrile | Scheme H |
| 302 | 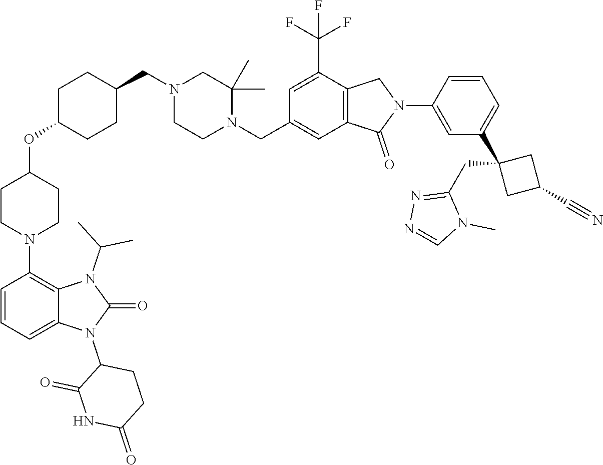 [SMILES: CC(C)n1c(=O)n(C2CCC(=O)NC2=O)c2cccc(N3CCC(O[C@H]4CC[C@H](CN5CCN(Cc6cc7c(c(C(F)(F)F)c6)CN(c6cccc([C@]8(Cc9nncn9C)C[C@@H](C#N)C8)c6)C7=O)C(C)(C)C5)CC4)CC3)c21] | (1r,3r)-3-(3-{6-[(2,2-dimethyl-4- {[(1r,4r)-4-({1-[1-(2,6- dioxopiperidin-3-yl)-2-oxo-3- (propan-2-yl)-2,3-dihydro-1H-1,3- benzodiazol-4-yl]piperidin-4- yl}oxy)cyclohexyl]methyl}piperazin- 1-yl)methyl]-1-oxo-4- (trifluoromethyl)-2,3-dihydro-1H- isoindol-2-yl}phenyl)-3-[(4-methyl- 4H-1,2,4-triazol-3- yl)methyl]cyclobutane-1-carbonitrile | Scheme H |
| 303 | 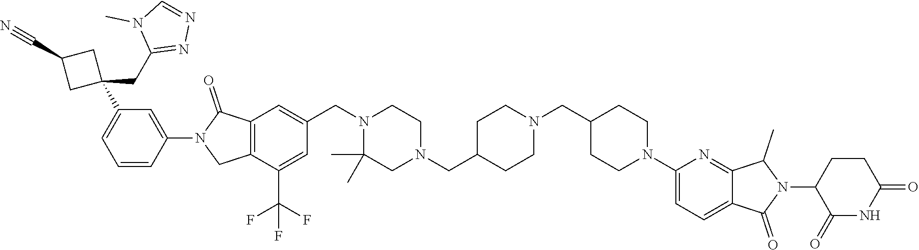 [SMILES: CC1c2nc(N3CCC(CN4CCC(CN5CCN(Cc6cc7c(c(C(F)(F)F)c6)CN(c6cccc([C@]8(Cc9nncn9C)C[C@@H](C#N)C8)c6)C7=O)C(C)(C)C5)CC4)CC3)ccc2C(=O)N1C1CCC(=O)NC1=O] | (1r,3r)-3-(3-{6-[(4-{[1-({1-[6-(2,6- dioxopiperidin-3-yl)-7-methyl-5-oxo- 5H,6H,7H-pyrrolo[3,4-b]pyridin-2- yl]piperidin-4-yl}methyl)piperidin-4- yl]methyl}-2,2-dimethylpiperazin-1- yl)methyl]-1-oxo-4- (trifluoromethyl)-2,3-dihydro-1H- isoindol-2-yl}phenyl)-3-[(4-methyl- 4H-1,2,4-triazol-3- yl)methyl]cyclobutane-1-carbonitrile | Scheme H |
| 304 | 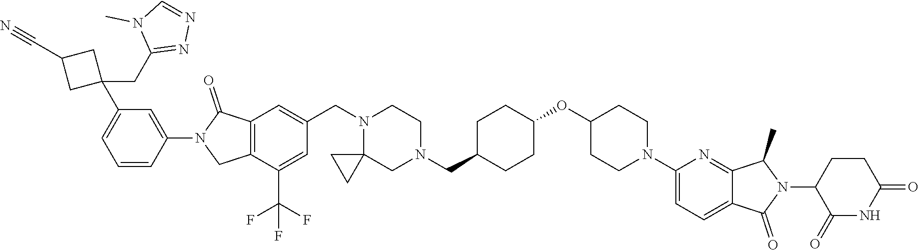 [SMILES: C[C@@H]1c2nc(N3CCC(O[C@H]4CC[C@H](CN5CCN(Cc6cc7c(c(C(F)(F)F)c6)CN(c6cccc(C8(Cc9nncn9C)CC(C#N)C8)c6)C7=O)C6(CC6)C5)CC4)CC3)ccc2C(=O)N1C1CCC(=O)NC1=O] | (1r***,3r***)-3-[(4-methyl-4H- 1,2,4-triazol-3-yl)methyl]-3-(3-{1- oxo-6-[(7-{[(1r**,4r**)-4-({1- [(7R*)-6-(2,6-dioxopiperidin-3-yl)-7- methyl-5-oxo-5H,6H,7H-pyrrolo[3,4- b]pyridin-2-yl]piperidin-4- yl}oxy)cyclohexyl]methyl}-4,7- diazaspiro[2.5]octan-4-yl)methyl]-4- (trifluoromethyl)-2,3-dihydro-1H- isoindol-2-yl}phenyl)cyclobutane-1- carbonitrile | Scheme H |
| 305 | 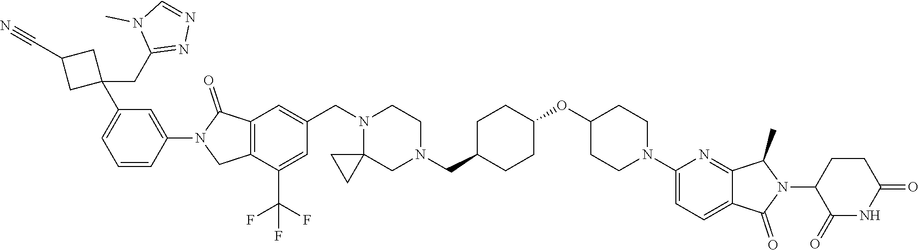 [SMILES: C[C@@H]1c2nc(N3CCC(O[C@H]4CC[C@H](CN5CCN(Cc6cc7c(c(C(F)(F)F)c6)CN(c6cccc(C8(Cc9nncn9C)CC(C#N)C8)c6)C7=O)C6(CC6)C5)CC4)CC3)ccc2C(=O)N1C1CCC(=O)NC1=O] | (1r***,3r***)-3-[(4-methyl-4H- 1,2,4-triazol-3-yl)methyl]-3-(3-{1- oxo-6-[(7-{[(1r**,4r**)-4-({1- [(7R*)-6-(2,6-dioxopiperidin-3-yl)-7- methyl-5-oxo-5H,6H,7H-pyrrolo[3,4- b]pyridin-2-yl]piperidin-4- yl}oxy)cyclohexyl]methyl}-4,7- diazaspiro[2.5]octan-4-yl)methyl]-4- (trifluoromethyl)-2,3-dihydro-1H- isoindol-2-yl}phenyl)cyclobutane-1- carbonitrile | Scheme H |
| 306 | 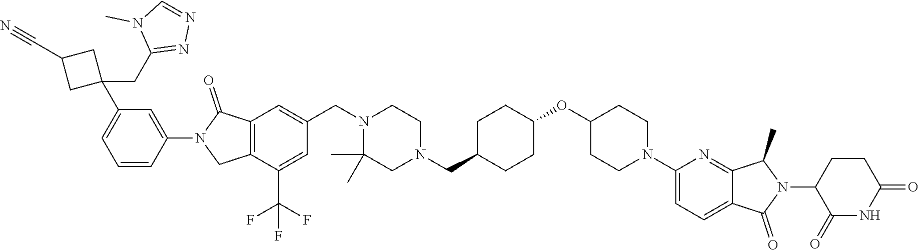 [SMILES: C[C@@H]1c2nc(N3CCC(O[C@H]4CC[C@H](CN5CCN(Cc6cc7c(c(C(F)(F)F)c6)CN(c6cccc(C8(Cc9nncn9C)CC(C#N)C8)c6)C7=O)C(C)(C)C5)CC4)CC3)ccc2C(=O)N1C1CCC(=O)NC1=O] | (1r***,3r***)-3-(3-{6-[(2,2- dimethyl-4-{[(1r**,4r**)-4-({1- [(7R*)-6-(2,6-dioxopiperidin-3-yl)-7- methyl-5-oxo-5H,6H,7H-pyrrolo[3,4- b]pyridin-2-yl]piperidin-4- yl}oxy)cyclohexyl]methyl}piperazin- 1-yl)methyl]-1-oxo-4- (trifluoromethyl)-2,3-dihydro-1H- isoindol-2-yl}phenyl)-3-[(4-methyl- 4H-1,2,4-triazol-3- yl)methyl]cyclobutane-1-carbonitrile | Scheme H |
| 307 | 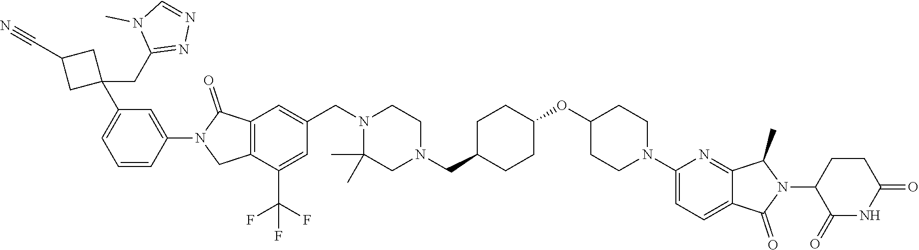 [SMILES: C[C@@H]1c2nc(N3CCC(O[C@H]4CC[C@H](CN5CCN(Cc6cc7c(c(C(F)(F)F)c6)CN(c6cccc(C8(Cc9nncn9C)CC(C#N)C8)c6)C7=O)C(C)(C)C5)CC4)CC3)ccc2C(=O)N1C1CCC(=O)NC1=O] | (1r***,3r***)-3-(3-{6-[(2,2- dimethyl-4-{[(1r**,4r**)-4-({1- [(7R*)-6-(2,6-dioxopiperidin-3-yl)-7- methyl-5-oxo-5H,6H,7H-pyrrolo[3,4- b]pyridin-2-yl]piperidin-4- yl}oxy)cyclohexyl]methyl}piperazin- 1-yl)methyl]-1-oxo-4- (trifluoromethyl)-2,3-dihydro-1H- isoindol-2-yl}phenyl)-3-[(4-methyl- 4H-1,2,4-triazol-3- yl)methyl]cyclobutane-1-carbonitrile | Scheme H |
| 308 | 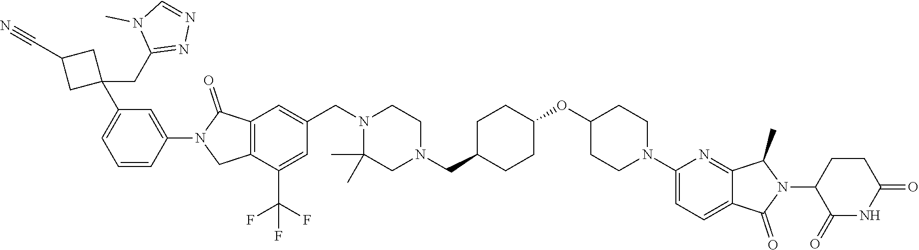 [SMILES: C[C@@H]1c2nc(N3CCC(O[C@H]4CC[C@H](CN5CCN(Cc6cc7c(c(C(F)(F)F)c6)CN(c6cccc(C8(Cc9nncn9C)CC(C#N)C8)c6)C7=O)C(C)(C)C5)CC4)CC3)ccc2C(=O)N1C1CCC(=O)NC1=O] | (1r***,3r***)-3-(3-{6-[(2,2- dimethyl-4-{[(1r**,4r**)-4-({1- [(7R*)-6-(2,6-dioxopiperidin-3-yl)-7- methyl-5-oxo-5H,6H,7H-pyrrolo[3,4- b]pyridin-2-yl]piperidin-4- yl}oxy)cyclohexyl]methyl}piperazin- 1-yl)methyl]-1-oxo-4- (trifluoromethyl)-2,3-dihydro-1H- isoindol-2-yl}phenyl)-3-[(4-methyl- 4H-1,2,4-triazol-3- yl)methyl]cyclobutane-1-carbonitrile | Scheme H |
| 309 | 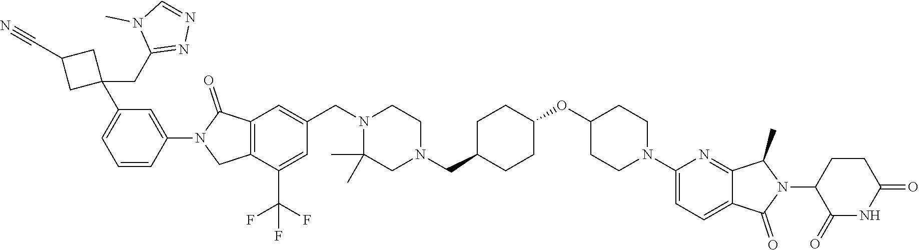 [SMILES: C[C@@H]1c2nc(N3CCC(O[C@H]4CC[C@H](CN5CCN(Cc6cc7c(c(C(F)(F)F)c6)CN(c6cccc(C8(Cc9nncn9C)CC(C#N)C8)c6)C7=O)C(C)(C)C5)CC4)CC3)ccc2C(=O)N1C1CCC(=O)NC1=O] | (1r***,3r***)-3-(3-{6-[(2,2- dimethyl-4-{[(1r**,4r**)-4-({1- [(7R*)-6-(2,6-dioxopiperidin-3-yl)-7- methyl-5-oxo-5H,6H,7H-pyrrolo[3,4- b]pyridin-2-yl]piperidin-4- yl}oxy)cyclohexyl]methyl}piperazin- 1-yl)methyl]-1-oxo-4- (trifluoromethyl)-2,3-dihydro-1H- isoindol-2-yl}phenyl)-3-[(4-methyl- 4H-1,2,4-triazol-3- yl)methyl]cyclobutane-1-carbonitrile | Scheme H |
| 310 | 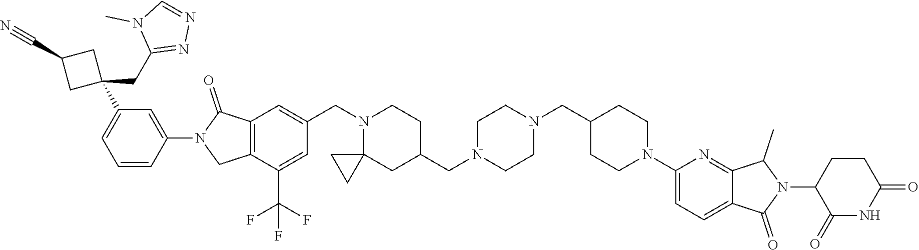 [SMILES: CC1c2nc(N3CCC(CN4CCN(CC5CCN(Cc6cc7c(c(C(F)(F)F)c6)CN(c6cccc([C@]8(Cc9nncn9C)C[C@@H](C#N)C8)c6)C7=O)C6(CC6)C5)CC4)CC3)ccc2C(=O)N1C1CCC(=O)NC1=O] | (1r,3r)-3-(3-{6-[(7-{[4-({1-[6-(2,6- dioxopiperidin-3-yl)-7-methyl-5-oxo- 5H,6H,7H-pyrrolo[3,4-b]pyridin-2- yl]piperidin-4-yl}methyl)piperazin-1- yl]methyl}-4-azaspiro[2.5]octan-4- yl)methyl]-1-oxo-4- (trifluoromethyl)-2,3-dihydro-1H- isoindol-2-yl}phenyl)-3-[(4-methyl- 4H-1,2,4-triazol-3- yl)methyl]cyclobutane-1-carbonitrile | Scheme H |
| 311 | 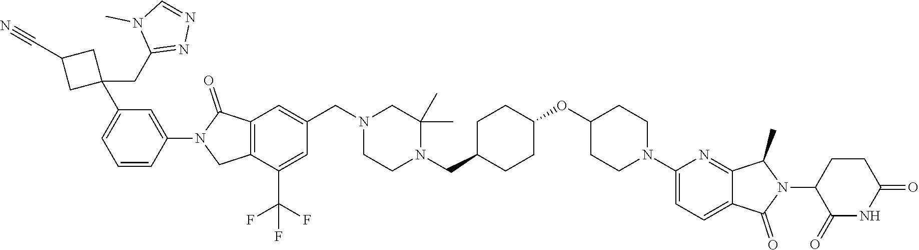 [SMILES: C[C@@H]1c2nc(N3CCC(O[C@H]4CC[C@H](CN5CCN(Cc6cc7c(c(C(F)(F)F)c6)CN(c6cccc(C8(Cc9nncn9C)CC(C#N)C8)c6)C7=O)CC5(C)C)CC4)CC3)ccc2C(=O)N1C1CCC(=O)NC1=O] | (1r***,3r***)-3-(3-{6-[(3,3- dimethyl-4-{[(1r**,4r**)-4-({1- [(7R*)-6-(2,6-dioxopiperidin-3-yl)-7- methyl-5-oxo-5H,6H,7H-pyrrolo[3,4- b]pyridin-2-yl]piperidin-4- yl}oxy)cyclohexyl]methyl}piperazin- 1-yl)methyl]-1-oxo-4- (trifluoromethyl)-2,3-dihydro-1H- isoindol-2-yl}phenyl)-3-[(4-methyl- 4H-1,2,4-triazol-3- yl)methyl]cyclobutane-1-carbonitrile | Scheme H |
| 312 | 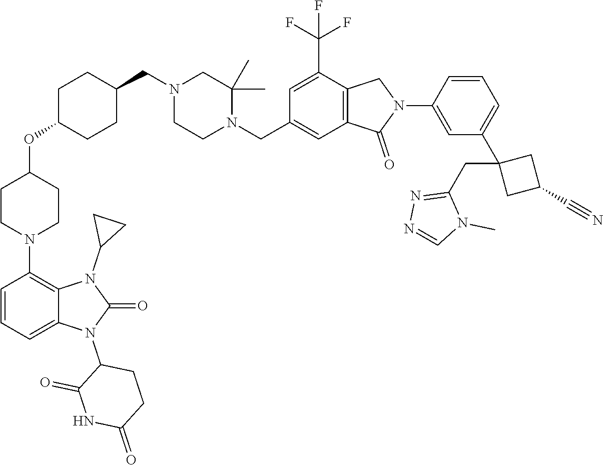 | (1r**,3r**)-3-(3-{6-[(2,2-dimethyl- 4-{[(1r*,4r*)-4-({1-[3-cyclopropyl- 1-(2,6-dioxopiperidin-3-yl)-2-oxo- 2,3-dihydro-1H-1,3-benzodiazol-4- yl]piperidin-4- yl}oxy)cyclohexyl]methyl}piperazin- 1-yl)methyl]-1-oxo-4- (trifluoromethyl)-2,3-dihydro-1H- isoindol-2-yl}phenyl)-3-[(4-methyl- 4H-1,2,4-triazol-3- yl)methyl]cyclobutane-1-carbonitrile | Scheme H |
| 313 | 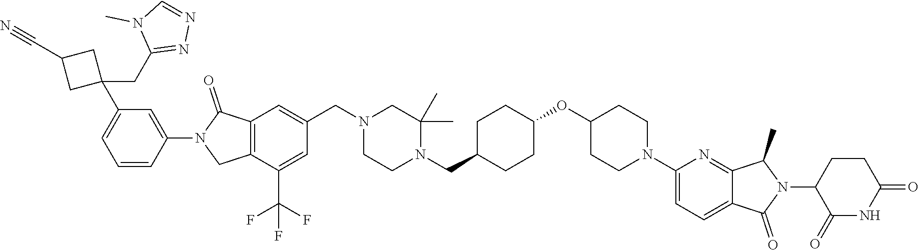 [SMILES: C[C@@H]1c2nc(N3CCC(O[C@H]4CC[C@H](CN5CCN(Cc6cc7c(c(C(F)(F)F)c6)CN(c6cccc(C8(Cc9nncn9C)CC(C#N)C8)c6)C7=O)CC5(C)C)CC4)CC3)ccc2C(=O)N1C1CCC(=O)NC1=O] | (1r***,3r***)-3-(3-{6-[(3,3- dimethyl-4-{[(1r**,4r**)-4-({1- [(7R*)-6-(2,6-dioxopiperidin-3-yl)-7- methyl-5-oxo-5H,6H,7H-pyrrolo[3,4- b]pyridin-2-yl]piperidin-4- yl}oxy)cyclohexyl]methyl}piperazin- 1-yl)methyl]-1-oxo-4- (trifluoromethyl)-2,3-dihydro-1H- isoindol-2-yl}phenyl)-3-[(4-methyl- 4H-1,2,4-triazol-3- yl)methyl]cyclobutane-1-carbonitrile | Scheme H |
| 314 | 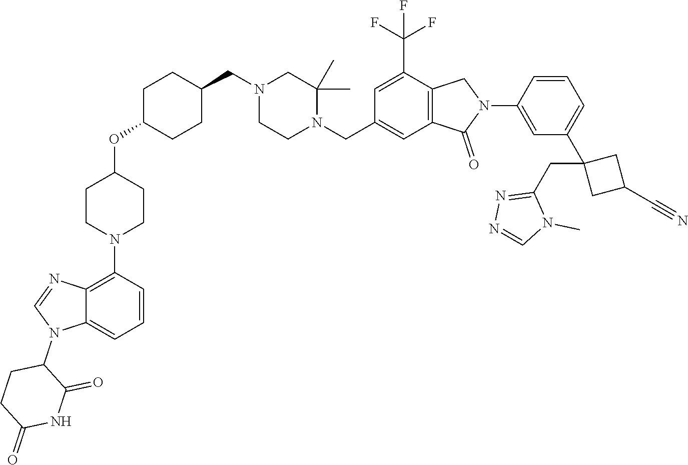 [SMILES: Cn1cnnc1CC1(c2cccc(N3Cc4c(cc(CN5CCN(C[C@H]6CC[C@H](OC7CCN(c8cccc9c8ncn9C8CCC(=O)NC8=O)CC7)CC6)CC5(C)C)cc4C(F)(F)F)C3=O)c2)CC(C#N)C1] | (1r**,3r**)-3-(3-{6-[(2,2-dimethyl- 4-{[(1r*,4r*)-4-({1-[1-(2,6- dioxopiperidin-3-yl)-1H-1,3- benzodiazol-4-yl]piperidin-4- yl}oxy)cyclohexyl]methyl}piperazin- 1-yl)methyl]-1-oxo-4- (trifluoromethyl)-2,3-dihydro-1H- isoindol-2-yl}phenyl)-3-[(4-methyl- 4H-1,2,4-triazol-3- yl)methyl]cyclobutane-1-carbonitrile | Scheme H |
| 315 | 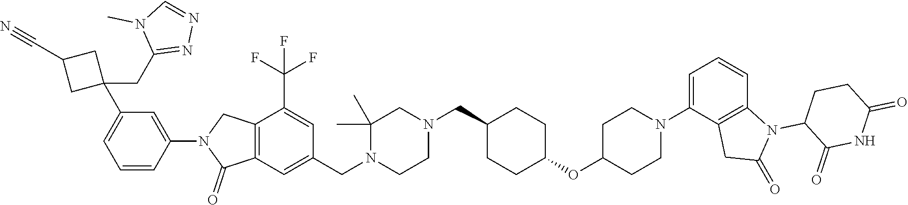 [SMILES: Cn1cnnc1CC1(c2cccc(N3Cc4c(cc(CN5CCN(C[C@H]6CC[C@H](OC7CCN(c8cccc9c8CC(=O)N9C8CCC(=O)NC8=O)CC7)CC6)CC5(C)C)cc4C(F)(F)F)C3=O)c2)CC(C#N)C1] | (1r**,3r**)-3-(3-{6-[(2,2-dimethyl- 4-{[(1r*,4r*)-4-({1-[1-(2,6- dioxopiperidin-3-yl)-2-oxo-2,3- dihydro-1H-indol-4-yl]piperidin-4- yl}oxy)cyclohexyl]methyl}piperazin- 1-yl)methyl]-1-oxo-4- (trifluoromethyl)-2,3-dihydro-1H- isoindol-2-yl}phenyl)-3-[(4-methyl- 4H-1,2,4-triazol-3- yl)methyl]cyclobutane-1-carbonitrile | Scheme H |
| 316 | 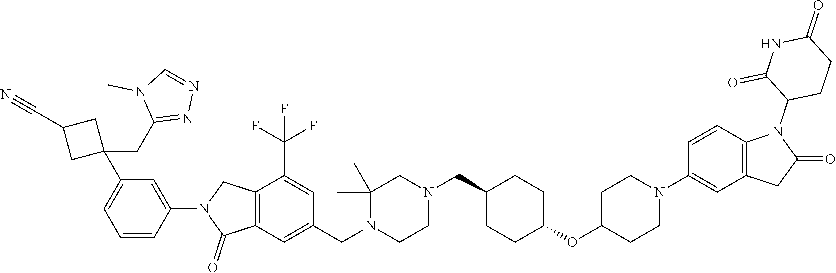 [SMILES: Cn1cnnc1CC1(c2cccc(N3Cc4c(cc(CN5CCN(C[C@H]6CC[C@H](OC7CCN(c8ccc9c(c8)CC(=O)N9C8CCC(=O)NC8=O)CC7)CC6)CC5(C)C)cc4C(F)(F)F)C3=O)c2)CC(C#N)C1] | (1r**,3r**)-3-(3-{6-[(2,2-dimethyl- 4-{[(1r*,4r*)-4-({1-[1-(2,6- dioxopiperidin-3-yl)-2-oxo-2,3- dihydro-1H-indol-5-yl]piperidin-4- yl}oxy)cyclohexyl]methyl}piperazin- 1-yl)methyl]-1-oxo-4- (trifluoromethyl)-2,3-dihydro-1H- isoindol-2-yl}phenyl)-3-[(4-methyl- 4H-1,2,4-triazol-3- yl)methyl]cyclobutane-1-carbonitrile | Scheme H |
| 317 | 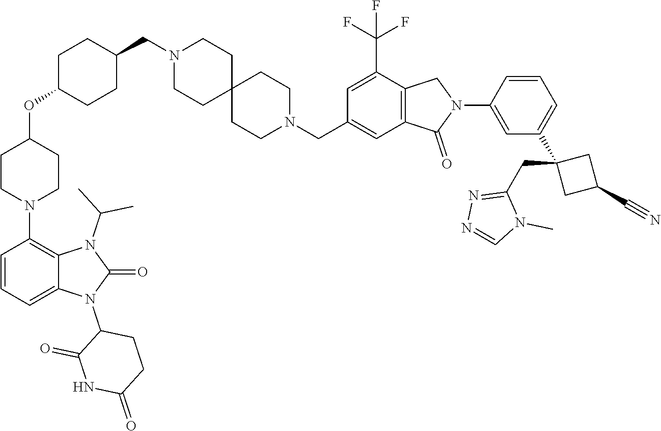 [SMILES: CC(C)n1c(=O)n(C2CCC(=O)NC2=O)c2cccc(N3CCC(O[C@H]4CC[C@H](CN5CCC6(CCN(Cc7cc8c(c(C(F)(F)F)c7)CN(c7cccc([C@]9(Cc%10nncn%10C)C[C@@H](C#N)C9)c7)C8=O)CC6)CC5)CC4)CC3)c21] | (1r,3r)-3-[(4-methyl-4H-1,2,4-triazol- 3-yl)methyl]-3-(3-{1-oxo-6-[(9- {[(1r,4r)-4-({1-[1-(2,6- dioxopiperidin-3-yl)-2-oxo-3- (propan-2-yl)-2,3-dihydro-1H-1,3- benzodiazol-4-yl]piperidin-4- yl}oxy)cyclohexyl]methyl}-3,9- diazaspiro[5.5]undecan-3-yl)methyl]- 4-(trifluoromethyl)-2,3-dihydro-1H- isoindol-2-yl}phenyl)cyclobutane-1- carbonitrile | Scheme H |
| 318 | 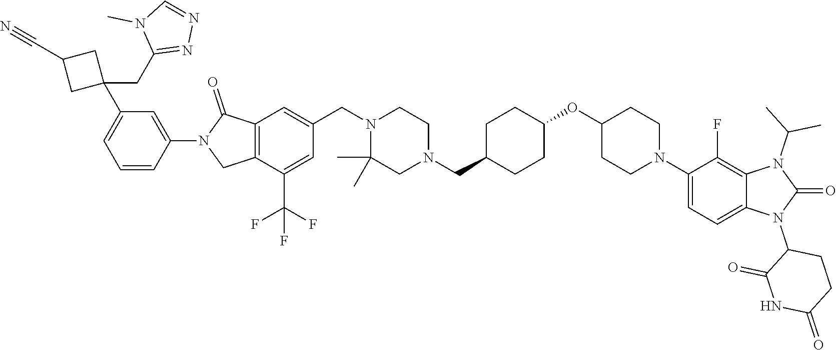 [SMILES: CC(C)n1c(=O)n(C2CCC(=O)NC2=O)c2ccc(N3CCC(O[C@H]4CC[C@H](CN5CCN(Cc6cc7c(c(C(F)(F)F)c6)CN(c6cccc(C8(Cc9nncn9C)CC(C#N)C8)c6)C7=O)C(C)(C)C5)CC4)CC3)c(F)c21] | (1r**,3r**)-3-(3-{6-[(2,2-dimethyl- 4-{[(1r*,4r*)-4-({1-[1-(2,6- dioxopiperidin-3-yl)-4-fluoro-2-oxo- 3-(propan-2-yl)-2,3-dihydro-1H-1,3- benzodiazol-5-yl]piperidin-4- yl}oxy)cyclohexyl]methyl}piperazin- 1-yl)methyl]-1-oxo-4- (trifluoromethyl)-2,3-dihydro-1H- isoindol-2-yl}phenyl)-3-[(4-methyl- 4H-1,2,4-triazol-3- yl)methyl]cyclobutane-1-carbonitrile | Scheme H |
| 319 | 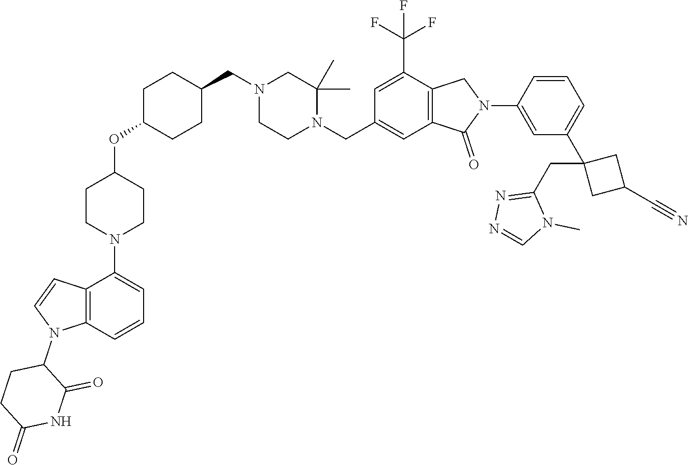 [SMILES: Cn1cnnc1CC1(c2cccc(N3Cc4c(cc(CN5CCN(C[C@H]6CC[C@H](OC7CCN(c8cccc9c8ccn9C8CCC(=O)NC8=O)CC7)CC6)CC5(C)C)cc4C(F)(F)F)C3=O)c2)CC(C#N)C1] | (1r**,3r**)-3-(3-{6-[(2,2-dimethyl- 4-{[(1r*,4r*)-4-({1-[1-(2,6- dioxopiperidin-3-yl)-1H-indol-4- yl]piperidin-4- yl}oxy)cyclohexyl]methyl}piperazin- 1-yl)methyl]-1-oxo-4- (trifluoromethyl)-2,3-dihydro-1H- isoindol-2-yl}phenyl)-3-[(4-methyl- 4H-1,2,4-triazol-3- yl)methyl]cyclobutane-1-carbonitrile | Scheme H |
| 320 | 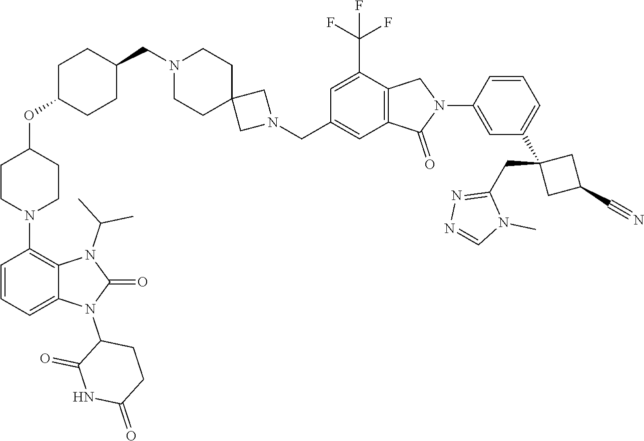 [SMILES: CC(C)n1c(=O)n(C2CCC(=O)NC2=O)c2cccc(N3CCC(O[C@H]4CC[C@H](CN5CCC6(CC5)CN(Cc5cc7c(c(C(F)(F)F)c5)CN(c5cccc([C@]8(Cc9nncn9C)C[C@@H](C#N)C8)c5)C7=O)C6)CC4)CC3)c21] | (1r,3r)-3-[(4-methyl-4H-1,2,4-triazol- 3-yl)methyl]-3-(3-{1-oxo-6-[(7- {[(1r,4r)-4-({1-[1-(2,6- dioxopiperidin-3-yl)-2-oxo-3- (propan-2-yl)-2,3-dihydro-1H-1,3- benzodiazol-4-yl]piperidin-4- yl}oxy)cyclohexyl]methyl}-2,7- diazaspiro[3.5]nonan-2-yl)methyl]-4- (trifluoromethyl)-2,3-dihydro-1H- isoindol-2-yl}phenyl)cyclobutane-1- carbonitrile | Scheme H |
| 321 | 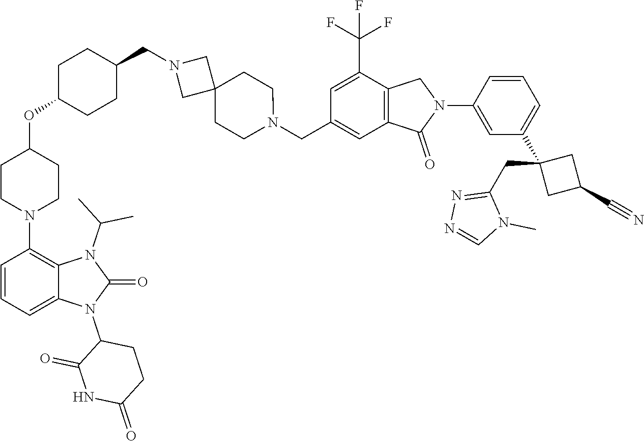 [SMILES: CC(C)n1c(=O)n(C2CCC(=O)NC2=O)c2cccc(N3CCC(O[C@H]4CC[C@H](CN5CC6(CCN(Cc7cc8c(c(C(F)(F)F)c7)CN(c7cccc([C@]9(Cc%10nncn%10C)C[C@@H](C#N)C9)c7)C8=O)CC6)C5)CC4)CC3)c21] | (1r,3r)-3-[(4-methyl-4H-1,2,4-triazol- 3-yl)methyl]-3-(3-{1-oxo-6-[(2- {[(1r,4r)-4-({1-[1-(2,6- dioxopiperidin-3-yl)-2-oxo-3- (propan-2-yl)-2,3-dihydro-1H-1,3- benzodiazol-4-yl]piperidin-4- yl}oxy)cyclohexyl]methyl}-2,7- diazaspiro[3.5]nonan-7-yl)methyl]-4- (trifluoromethyl)-2,3-dihydro-1H- isoindol-2-yl}phenyl)cyclobutane-1- carbonitrile | Scheme H |
| 322 | 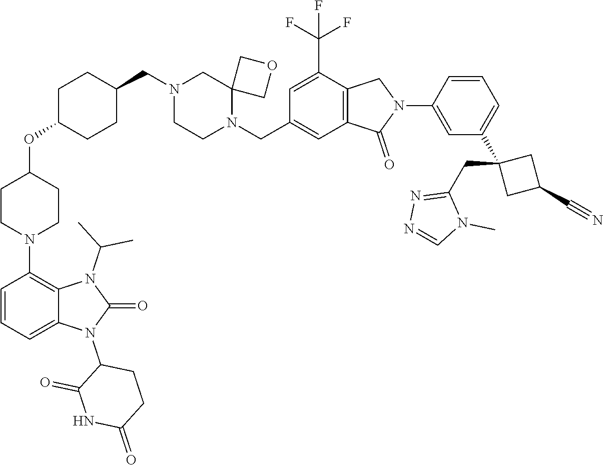 [SMILES: CC(C)n1c(=O)n(C2CCC(=O)NC2=O)c2cccc(N3CCC(O[C@H]4CC[C@H](CN5CCN(Cc6cc7c(c(C(F)(F)F)c6)CN(c6cccc([C@]8(Cc9nncn9C)C[C@@H](C#N)C8)c6)C7=O)C6(COC6)C5)CC4)CC3)c21] | (1r,3r)-3-[(4-methyl-4H-1,2,4-triazol- 3-yl)methyl]-3-(3-{1-oxo-6-[(8- {[(1r,4r)-4-({1-[1-(2,6- dioxopiperidin-3-yl)-2-oxo-3- (propan-2-yl)-2,3-dihydro-1H-1,3- benzodiazol-4-yl]piperidin-4- yl}oxy)cyclohexyl]methyl}-2-oxa- 5,8-diazaspiro[3.5]nonan-5- yl)methyl]-4-(trifluoromethyl)-2,3- dihydro-1H-isoindol-2- yl}phenyl)cyclobutane-1-carbonitrile | Scheme H |
| 323 | 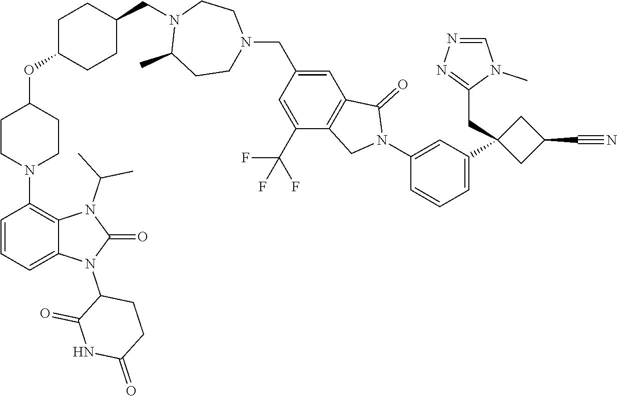 [SMILES: CC(C)n1c(=O)n(C2CCC(=O)NC2=O)c2cccc(N3CCC(O[C@H]4CC[C@H](CN5CCN(Cc6cc7c(c(C(F)(F)F)c6)CN(c6cccc([C@]8(Cc9nncn9C)C[C@@H](C#N)C8)c6)C7=O)CC[C@H]5C)CC4)CC3)c21] | (1r,3r)-3-[3-(6-{[(5R)-5-methyl-4- {[(1r,4r)-4-({1-[1-(2,6- dioxopiperidin-3-yl)-2-oxo-3- (propan-2-yl)-2,3-dihydro-1H-1,3- benzodiazol-4-yl]piperidin-4- yl}oxy)cyclohexyl]methyl}-1,4- diazepan-1-yl]methyl}-1-oxo-4- (trifluoromethyl)-2,3-dihydro-1H- isoindol-2-yl)phenyl]-3-[(4-methyl- 4H-1,2,4-triazol-3- yl)methyl]cyclobutane-1-carbonitrile | Scheme H |
| 324 | 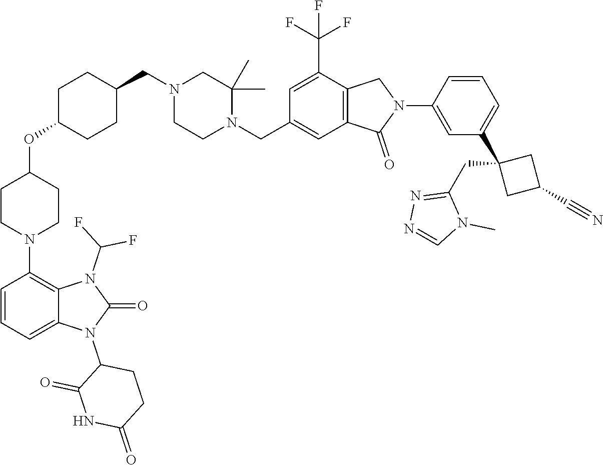 [SMILES: Cn1cnnc1C[C@]1(c2cccc(N3Cc4c(cc(CN5CCN(C[C@H]6CC[C@H](OC7CCN(c8cccc9c8n(C(F)F)c(=O)n9C8CCC(=O)NC8=O)CC7)CC6)CC5(C)C)cc4C(F)(F)F)C3=O)c2)C[C@H](C#N)C1] | (1r,3r)-3-(3-{6-[(2,2-dimethyl-4- {[(1r,4r)-4-({1-[3-(difluoromethyl)- 1-(2,6-dioxopiperidin-3-yl)-2-oxo- 2,3-dihydro-1H-1,3-benzodiazol-4- yl]piperidin-4- yl}oxy)cyclohexyl]methyl}piperazin- 1-yl)methyl]-1-oxo-4- (trifluoromethyl)-2,3-dihydro-1H- isoindol-2-yl}phenyl)-3-[(4-methyl- 4H-1,2,4-triazol-3- yl)methyl]cyclobutane-1-carbonitrile | Scheme H |
| 325 | 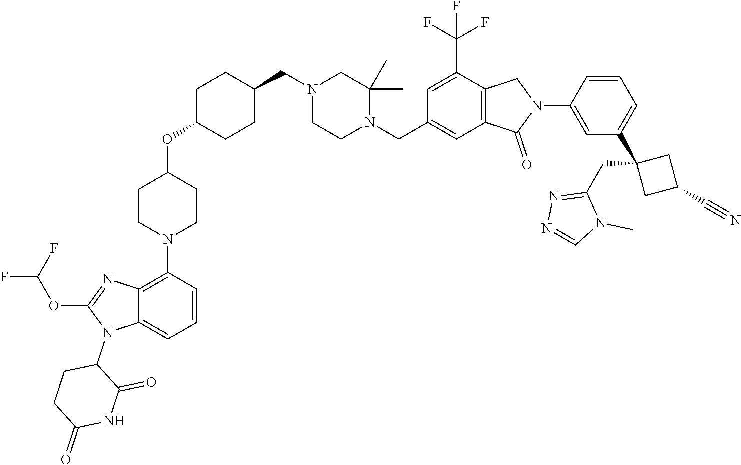 [SMILES: Cn1cnnc1C[C@]1(c2cccc(N3Cc4c(cc(CN5CCN(C[C@H]6CC[C@H](OC7CCN(c8cccc9c8nc(OC(F)F)n9C8CCC(=O)NC8=O)CC7)CC6)CC5(C)C)cc4C(F)(F)F)C3=O)c2)C[C@H](C#N)C1] | (1r,3r)-3-(3-{6-[(2,2-dimethyl-4- {[(1r,4r)-4-({1-[2-(difluoromethoxy)- 1-(2,6-dioxopiperidin-3-yl)-1H-1,3- benzodiazol-4-yl]piperidin-4- yl}oxy)cyclohexyl]methyl}piperazin- 1-yl)methyl]-1-oxo-4- (trifluoromethyl)-2,3-dihydro-1H- isoindol-2-yl}phenyl)-3-[(4-methyl- 4H-1,2,4-triazol-3- yl)methyl]cyclobutane-1-carbonitrile | Scheme H |
| 326 | 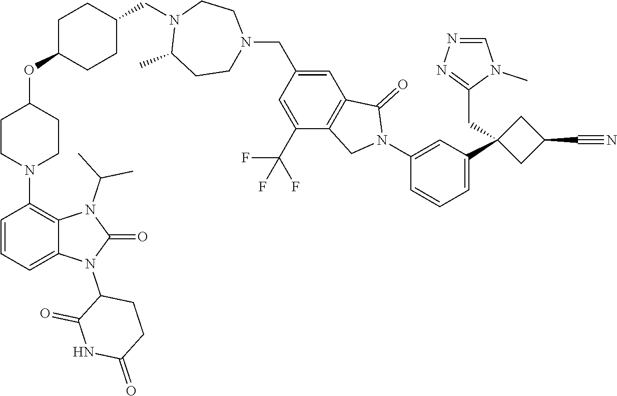 [SMILES: CC(C)n1c(=O)n(C2CCC(=O)NC2=O)c2cccc(N3CCC(O[C@H]4CC[C@H](CN5CCN(Cc6cc7c(c(C(F)(F)F)c6)CN(c6cccc([C@]8(Cc9nncn9C)C[C@H](C#N)C8)c6)C7=O)CC[C@@H]5C)CC4)CC3)c21] | (1r,3r)-3-[3-(6-{[(5S)-5-methyl-4- {[(1r,4r)-4-({1-[1-(2,6- dioxopiperidin-3-yl)-2-oxo-3- (propan-2-yl)-2,3-dihydro-1H-1,3- benzodiazol-4-yl]piperidin-4- yl}oxy)cyclohexyl]methyl}-1,4- diazepan-1-yl]methyl}-1-oxo-4- (trifluoromethyl)-2,3-dihydro-1H- isoindol-2-yl)phenyl]-3-[(4-methyl- 4H-1,2,4-triazol-3- yl)methyl]cyclobutane-1-carbonitrile | Scheme H |
| 327 | 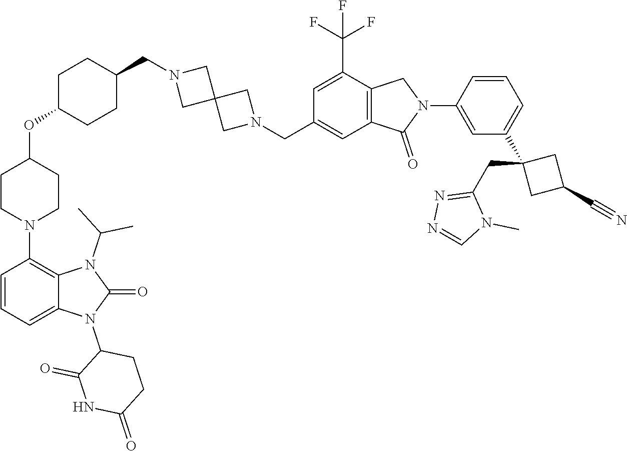 [SMILES: CC(C)n1c(=O)n(C2CCC(=O)NC2=O)c2cccc(N3CCC(O[C@H]4CC[C@H](CN5CC6(CN(Cc7cc8c(c(C(F)(F)F)c7)CN(c7cccc([C@]9(Cc%10nncn%10C)C[C@@H](C#N)C9)c7)C8=O)C6)C5)CC4)CC3)c21] | (1r,3r)-3-[(4-methyl-4H-1,2,4-triazol- 3-yl)methyl]-3-(3-{1-oxo-6-[(6- {[(1r,4r)-4-({1-[1-(2,6- dioxopiperidin-3-yl)-2-oxo-3- (propan-2-yl)-2,3-dihydro-1H-1,3- benzodiazol-4-yl]piperidin-4- yl}oxy)cyclohexyl]methyl}-2,6- diazaspiro[3.3]heptan-2-yl)methyl]- 4-(trifluoromethyl)-2,3-dihydro-1H- isoindol-2-yl}phenyl)cyclobutane-1- carbonitrile | Scheme H |
| 328 | 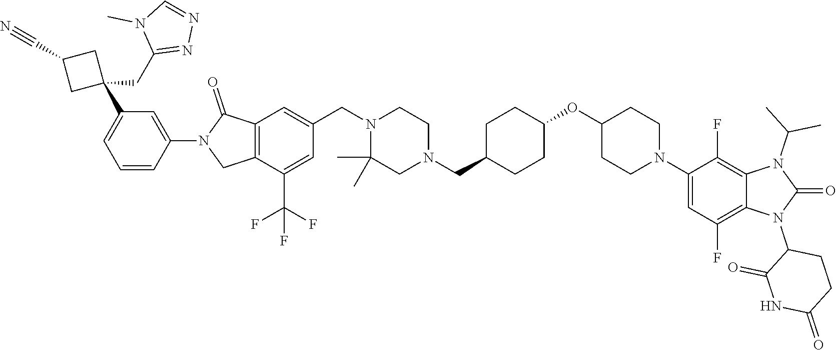 [SMILES: CC(C)n1c(=O)n(C2CCC(=O)NC2=O)c2c(F)cc(N3CCC(O[C@H]4CC[C@H](CN5CCN(Cc6cc7c(c(C(F)(F)F)c6)CN(c6cccc([C@]8(Cc9nncn9C)C[C@@H](C#N)C8)c6)C7=O)C(C)(C)C5)CC4)CC3)c(F)c21] | (1r,3r)-3-(3-{6-[(2,2-dimethyl-4- {[(1r,4r)-4-({1-[1-(2,6- dioxopiperidin-3-yl)-4,7-difluoro-2- oxo-3-(propan-2-yl)-2,3-dihydro-1H- 1,3-benzodiazol-5-yl]piperidin-4- yl}oxy)cyclohexyl]methyl}piperazin- 1-yl)methyl]-1-oxo-4- (trifluoromethyl)-2,3-dihydro-1H- isoindol-2-yl}phenyl)-3-[(4-methyl- 4H-1,2,4-triazol-3- yl)methyl]cyclobutane-1-carbonitrile | Scheme H |

TABLE 2

| Ex. No. | ¹H NMR |
|---|---|
| 23 | ¹H NMR (400 MHz, DMSO) δ 0.95-1.28 (m, 3 H) 1.54-1.65 (m, 1 H) 1.71- |
| | 1.83 (m, 6 H) 1.97-2.05 (m, 1 H) 2.35 (d, J = 7.6 Hz, 2 H) 2.54-2.61 (m, 2 H) |
| | 2.89 (s, 3 H) 2.92-2.97 (m, 2 H) 3.02 (s, 4 H) 3.30 (d, J = 3.6 Hz, 4 H) 3.51 (s, |
| | 2 H) 3.98-4.08 (m, 2 H) 4.89 (d, J = 6.0 Hz, 2 H) 4.96 (d, J = 5.6 Hz, 4 H) 5.07 |
| | (dd, J = 12.8, 5.2 Hz, 1 H) 6.75 (d, J = 7.6 Hz, 1 H) 7.23 (d, J = 8.8 Hz, 1 H) |
| | 7.29-7.37 (m, 3 H) 7.42-7.54 (m, 2 H) 7.65 (d, J = 8.8 Hz, 1 H) 7.86-7.93 (m, 1 |
| | H) 8.19 (s, 1 H) 11.10 (s, 1 H) |
| 24 | ¹H NMR (400 MHz, DMSO) δ 11.00 (s, 1H), 8.16-8.23 (m, 2H), 7.98 (s, 1H), |
| | 7.93 (s, 1H), 7.89 (br d, J = 9.2 Hz, 1H), 7.62-7.69 (m, 1H), 7.40 (s, 1H), 7.33- |
| | 7.38 (m, 1H), 7.27 (d, J = 8.8 Hz, 1H), 6.77 (d, J = 7.6 Hz, 1H), 5.07-5.14 (m, |
| | 3H), 4.97 (d, J = 6.0 Hz, 2H), 4.89 (d, J = 6.0 Hz, 2H), 4.41 (d, J = 17.6 Hz, |
| | 1H), 4.25 (d, J = 17.6 Hz, 1H), 3.69 (s, 2H), 3.60-3.66 (m, 1H), 3.52 (s, 2H), |
| | 3.23-3.28 (m, 3H), 2.95 (br s, 1H), 2.90 (s, 3H), 2.88 (br d, J = 4.4 Hz, 2H), |
| | 2.60 (br s, 2H), 2.57 (br s, 2H), 2.38-2.46 (m, 6H), 2.07 (br d, J = 6.8 Hz, 2H), |
| | 1.98-2.02 (m, 1H), 1.90-1.97 (m, 4H), 1.73-1.80 (m, 2H), 1.57-1.65 (m, 2H), |
| | 1.39-1.46 (m, 1H), 1.11-1.20 (m, 2H), 0.82-0.93 ppm (m, 2H) |
| 25 | ¹H NMR (400 MHz, DMSO) δ 10.98 (s, 1H), 8.19 (s, 1H), 8.18 (s, 2H), 7.94 |
| | (s, 1H), 7.92-7.84 (m, 2H), 7.46 (d, J = 8.4 Hz, 1H), 7.41 (s, 1H), 7.36 (t, J = |
| | 8.0 Hz, 1H), 7.22-7.10 (m, 1H), 6.77 (d, J = 7.6 Hz, 1H), 5.15-5.03 (m, 3H), |
| | 4.97 (d, J = 6.0 Hz, 2H), 4.89 (d, J = 6.0 Hz, 2H), 4.48 (d, J = 17.2 Hz, 1H), |
| | 4.31 (d, J = 16.8 Hz, 1H), 3.80 (s, 2H), 3.56-3.44 (m, 6H), 2.99 (s, 4H), 2.91 |
| | (s, 3H), 2.77 (t, J = 12.0 Hz, 2H), 2.44 (s, 2H), 2.31-2.24 (m, 3H), 2.13 (d, |
| | J = 7.2 Hz, 2H), 2.01-1.94 (m, 1H), 1.79 (d, J = 12.0 Hz, 2H), 1.70 (s, 5H), |
| | 1.32-1.21 (m, 2H) |
| 27 | ¹H NMR (400 MHz, DMSO-d6) δ 11.17 (s, 1H), 8.19 (s, 1H), 7.98 (s, 1H), |
| | 7.93 (s, 1H), 7.88 (s, 1H), 7.64 (s, 1H), 7.39 (s, 1H), 7.34 (s, 1H), 7.29 (s, 1H), |
| | 7.24-7.19 (m, 1H), 6.78-6.73 (m, 1H), 5.13-5.02 (m, 3H), 4.98-4.94 (m, 2H), |
| | 4.90-4.86 (m, 2H), 3.75-3.67 (m, 2H), 3.52-3.50 (m, 2H), 3.48-3.44 (m, 5H), |
| | 2.94-2.86 (m, 4H), 2.44-2.38 (m, 5H), 2.07-1.95 (m, 1H), 1.52 (s, 8H) |
| 33 | ¹H NMR (400 MHz, DMSO-d6) δ 8.23-8.19 (m, 1H), 8.17-8.13 (m, 1H), |
| | 8.08-8.04 (m, 1H), 7.84 (d, J = 4.8 Hz, 2H), 7.81-7.76 (m, 2H), 7.41 (s, 1H), |
| | 6.81-6.76 (m, 1H), 5.13 (s, 2H), 5.08 (s, 5H), 3.98-3.91 (m, 2H), 3.72-3.65 |
| | (m, 2H), 3.22-3.13 (m, 2H), 2.95-2.91 (m, 3H), 2.91-2.84 (m, 2H), 2.82-2.71 |
| | (m, 2H), 2.54-2.41 (m, 2H), 2.21-2.10 (m, 1H), 2.05-1.89 (m, 4H) |
| 34 | ¹H NMR (400 MHz, DMSO-d6) δ 8.36 (s, 1H), 8.21 (s, 1H), 8.16 (s, 1H), 8.08 |
| | (s, 1H), 7.82-7.73 (m, 2H), 7.53-7.50 (m, 1H), 7.48-7.39 (m, 3H), 6.81-6.76 |
| | (m, 1H), 5.20-5.12 (m, 3H), 5.12-5.06 (m, 4H), 4.55-4.43 (m, 2H), 4.09-4.01 |
| | (m, 2H), 3.70-3.64 (m, 2H), 3.29-3.20 (m, 2H), 2.96-2.76 (m, 6H), 2.63-2.44 |
| | (m, 3H), 2.23-2.14 (m, 1H), 2.03-1.87 (m, 4H) |
| 36 | ¹H NMR (400 MHz, DMSO-d6) δ 8.64 (s, 1H), 8.36-8.22 (m, 2H), 7.81-7.70 |
| | (m, 2H), 7.60 (s, 2H), 7.52-7.44 (m, 2H), 6.95-6.88 (m, 1H), 5.25-5.21 (m, |
| | 2H), 5.20-5.14 (m, 1H), 5.12-5.06 (m, 4H), 4.93-4.92 (m, 3H), 4.66 (s, 1H), |
| | 4.58-4.42 (m, 2H), 3.77 (s, 2H), 3.74-3.65 (m, 2H), 3.19-3.04 (m, 4H), 3.00- |
| | 2.86 (m, 1H), 2.86-2.75 (m, 1H), 2.61-2.44 (m, 1H), 2.30-2.14 (m, 3H), |
| | 2.13-1.97 (m, 2H) |
| 38 | ¹H NMR (400 MHz, DMSO-d6) δ 11.10 (s, 1H), 8.19 (s, 1H), 8.07 (s, 1H), |
| | 8.02 (s, 1H), 7.87 (s, 1H), 7.68-7.63 (m, 1H), 7.40-7.29 (m, 3H), 7.24 (dd, J = |
| | 1.9, 8.9 Hz, 1H), 6.78-6.73 (m, 1H), 5.12-5.01 (m, 3H), 4.99-4.93 (m, 2H), |
| | 4.91-4.86 (m, 2H), 4.06-3.77 (m, 5H), 3.10-2.98 (m, 3H), 2.93-2.80 (m, 5H), |
| | 2.79-2.64 (m, 2H), 2.63-2.58 (m, 1H), 2.05-1.87 (m, 3H), 1.43-1.28 (m, 2H) |
| 40 | ¹H NMR (400 MHz, DMSO-d6) δ 8.42 (s, 1H), 8.20 (s, 1H), 8.12 (s, 1H), 8.04 |
| | (s, 1H), 7.78 (dd, J = 1.2, 8.4 Hz, 1H), 7.66 (d, J = 8.4 Hz, 1H), 7.47-7.39 (m, |
| | 2H), 7.34 (d, J = 2.0 Hz, 1H), 7.25-7.19 (m, 1H), 6.83-6.76 (m, 1H), 5.15- |
| | 5.12 (m, 2H), 5.12-5.06 (m, 6H), 4.10-4.03 (m, 2H), 4.02-3.97 (m, 2H), |
| | 3.71-3.65 (m, 2H), 3.16-2.96 (m, 5H), 2.94-2.90 (m, 3H), 2.90-2.65 (m, 4H), |
| | 2.51-2.37 (m, 2H), 2.17-2.06 (m, 1H), 1.96-1.67 (m, 7H), 1.50-1.35 (m, 4H) |
| 44 | ¹H NMR (400 MHz, DMSO-d6) δ 11.05 (s, 1H), 8.22-8.13 (s, 1H), 7.99 (s, |
| | 1H), 7.95-7.85 (m, 2H), 7.54 (d, J = 8.4 Hz, 1H), 7.43-7.31 (m, 2H), 7.02- |
| | 6.92 (m, 2H), 6.84 (dd, J = 1.6, 8.4 Hz, 1H), 6.79-6.74 (m, 1H), 5.14-5.06 (m, |
| | 2H), 5.05-4.99 (m, 1H), 4.99-4.94 (m, 2H), 4.89 (d, J = 6.0 Hz, 2H), 3.79- |
| | 3.60 (m, 3H), 3.53-3.48 (m, 3H), 2.92-2.82 (m, 4H), 2.63-2.54 (m, 5H), |
| | 2.45-2.28 (m, 5H), 2.06-1.94 (m, 3H), 1.84 (d, J = 10.8 Hz, 2H), 1.47-1.35 |
| | (m, 2H), 1.27-1.13 (m, 2H) |
| 47 | ¹H NMR (400 MHz, DMSO-d6) δ 11.12 (s, 1H), 8.19 (s, 1H), 8.15 (s, 1H), |
| | 7.98 (s, 1H), 7.92 (s, 1H), 7.87 (s, 1H), 7.83 (s, 1H), 7.42 (s, 1H), 7.39 (s, 1H), |
| | 7.37-7.31 (m, 2H), 6.76 (d, J = 7.8 Hz, 1H), 5.16-5.06 (m, 3H), 4.98-4.94 (m, |
| | 2H), 4.88-4.88 (m, 1H), 4.91-4.86 (m, 1H), 4.25-4.17 (m, 2H), 3.75-3.62 (m, |
| | 3H), 3.54-3.49 (m, 3H), 2.93 (s, 5H), 2.63-2.56 (m, 2H), 2.47-2.41 (m, 7H), |
| | 2.09-2.01 (m, 1H), 1.95-1.86 (m, 2H) |
| 48 | ¹H NMR (400 MHz, DMSO-d6) δ 8.44 (s, 1H), 8.20 (s, 1H), 8.10 (s, 1H), 8.01 |
| | (s, 1H), 7.80 (s, 1H), 7.43 (s, 1H), 7.39 (s, 1H), 7.25-7.18 (m, 1H), 6.83-6.76 |
| | (m, 3H), 5.15-5.04 (m, 6H), 3.85-3.79 (m, 3H), 3.75 (t, J = 6.8 Hz, 3H), 3.69- |
| | 3.66 (m, 2H), 3.05-2.85 (m, 7H), 2.85-2.79 (m, 4H), 2.78-2.76 (m, 1H), |
| | 2.75-2.69 (m, 3H), 2.68-2.65 (m, 2H), 1.89 (d, J = 12.2 Hz, 3H), 1.45-1.30 |
| | (m, 2H) |
| 49 | ¹H NMR (400 MHz, DMSO-d6) δ 11.07 (s, 1H), 8.19 (s, 1H), 7.96 (s, 1H), |
| | 7.91 (s, 1H), 7.87 (s, 1H), 7.65 (s, 1H), 7.41 (s, 1H), 7.38-7.30 (m, 2H), 7.28- |
| | 7.21 (m, 1H), 6.80-6.72 (m, 1H), 5.15-5.01 (m, 3H), 4.99-4.92 (m, 2H), |
| | 4.91-4.84 (m, 2H), 3.89-3.78 (m, 2H), 3.53-3.50 (m, 2H), 3.47-3.43 (m, 5H), |
| | 3.11-3.05 (m, 4H), 2.92-2.81 (m, 4H), 2.61-2.56 (m, 1H), 2.06-1.95 (m, 1H), |
| | 1.83-1.72 (m, 4H) |
| 54 | ¹H NMR (400 MHz, DMSO-d6) δ 10.86 (s, 1H), 8.4 (s, 1H), 8.47 (s, 1H), 8.18 |
| | (s 1H), 8.16-8.13 (m, 1H), 7.97 (s, 1H), 7.93 (s, 1H), 7.88 (s, 1H), 7.42-7.31 |
| | (m, 2H), 7.15 (s, 3H), 6.80-6.73 (m, 1H), 5.13-5.06 (m, 2H), 4.99-4.93 (m, |
| | 2H), 4.88 (d, J = 6.1 Hz, 2H), 4.78-4.70 (m, 1H), 3.60 (d, J = 12.0 Hz, 6H), |
| | 3.55-3.49 (m, 4H), 2.91-2.87 (m, 3H), 2.81-2.73 (m, 1H), 2.68-2.58 (m, 3H), |
| | 2.44-2.38 (m, 4H), 2.20-2.14 (m, 2H), 2.11 (d, J = 4.4 Hz, 1H), 2.04-1.97 (m, |
| | 1H), 1.85-1.72 (m, 2H), 1.71-1.57 (m, 1H), 1.26-1.12 (m, 2H) |
| 55 | ¹H NMR (400 MHz, DMSO-d6) δ 8.35-8.29 (m, 1H), 8.23-8.18 (m, 1H), |
| | 8.14-8.09 (m, 1H), 8.06-8.02 (m, 1H), 7.81-7.75 (m, 1H), 7.60-7.55 (m, 1H), |
| | 7.52-7.47 (m, 1H), 7.46-7.38 (m, 2H), 6.82-6.76 (m, 1H), 6.41-6.35 (m, 1H), |
| | 5.14-5.06 (m, 6H), 4.47-4.43 (m, 2H), 4.02-3.93 (m, 4H), 3.71-3.64 (m, 2H), |
| | 3.61-3.53 (m, 2H), 3.19-3.08 (m, 2H), 2.92 (s, 3H), 2.71-2.65 (m, 2H), 2.47- |
| | 2.35 (m, 2H), 2.09-1.96 (m, 1H), 1.78-1.69 (m, 2H), 1.57-1.43 (m, 2H) |
| 57 | ¹H NMR (400 MHz, DMSO-d6) δ 8.44 (s, 1H), 8.20 (s, 1H), 8.10 (s, 1H), 8.00 |
| | (s, 1H), 7.78 (s, 1H), 7.43 (s, 1H), 7.39 (s 1H), 7.21 (s, 1H), 6.83-6.76 (m, |
| | 3H), 5.15-5.04 (m, 6H), 3.85-3.79 (m, 3H), 3.75 (t, J = 6.8 Hz, 3H), 3.69- |
| | 3.66 (m, 2H), 3.05-2.85 (m, 7H), 2.85-2.79 (m, 4H), 2.78-2.76 (m, 1H), |
| | 2.75-2.69 (m, 3H), 2.68-2.65 (m, 2H), 1.89 (d, J = 12.4 Hz, 3H), 1.45-1.30 |
| | (m, 2H) |
| 61 | ¹H NMR (400 MHz, DMSO-d6) δ 11.09 (s, 1H), 8.19 (s, 1H), 8.15 (s, 1H), |
| | 7.98 (s, 1H), 7.93 (s 1H), 7.87 (s, 1H), 7.64 (s, 1H), 7.41-7.38 (m, 1H), 7.38- |
| | 7.31 (m, 2H), 7.26 (d, J = 8.4 Hz, 1H), 6.80-6.73 (m, 1H), 5.12-5.03 (m, 3H), |
| | 4.99-4.95 (m, 2H), 4.89 (d, J = 6.0 Hz, 2H), 3.72-3.60 (m, 3H), 3.52 (s, 3H), |
| | 2.93-2.87 (m, 4H), 2.86-2.77 (m, 3H), 2.62-2.54 (m, 4H), 2.40-2.24 (m, 4H), |
| | 2.05-1.95 (m, 3H), 1.69-1.61 (m, 2H), 1.53-1.43 (m, 2H), 1.31-1.09 (m, 6H) |
| 72 | ¹H NMR (400 MHz, DMSO) δ 11.07 (s, 1H), 8.18 (s, 1H), 8.14 (s, 3H), 7.99 |
| | (s, 1H), 7.93 (s, 1H), 7.87 (d, J = 7.2 Hz, 1H), 7.68 (d, J = 8.4 Hz, 1H), 7.40 (s, |
| | 1H), 7.38-7.33 (m, 2H), 7.25 (d, J = 8.8 Hz, 1H), 6.80 (d, J = 7.3 Hz, 1H), |
| | 5.12-5.03 (m, 3H), 4.97 (d, J = 6.0 Hz, 2H), 4.89 (d, J = 6.0 Hz, 2H), 3.74- |
| | 3.69 (m, 4H), 3.51 (s, 2H), 3.16 (d, J = 3.6 Hz, 4H), 3.08-3.02 (m, 4H), 2.92 |
| | (s, 6H), 2.89-2.83 (m, 2H), 2.77 (d, J = 8.0 Hz, 2H), 2.30-2.24 (m, 2H), 2.20 |
| | (d, J = 6.0 Hz, 3H), 2.05-1.93 (m, 3H), 1.85-1.51 (m, 13H), 1.36-1.10 (m, |
| | 5H), 0.85-0.79 (m, 3H), 0.60-0.49 (m, 1H) |
| 73 | ¹H NMR (400 MHz, DMSO) δ 11.08 (s, 1H), 8.20-8.15 (m, 3H), 7.88 (d, J = |
| | 8.4 Hz, 1H), 7.67 (d, J = 8.4 Hz, 1H), 7.43 (s, 1H), 7.39 (s, 1H), 7.37-7.30 |
| | (m, 3H), 7.25 (d, J = 8.8 Hz, 1H), 6.75 (d, J = 7.6 Hz, 1H), 5.10-5.03 (m, |
| | 1H), 4.98-4.93 (m, 4H), 4.88 (d, J = 6.0 Hz, 2H), 3.95-3.89 (m, 2H), 3.51 (s, |
| | 3H), 2.91-2.81 (m, 8H), 2.60 (s, 2H), 2.46 (s, 2H), 2.43-2.36 (m, 3H), 2.30- |
| | 2.09 (m, 8H), 2.05-1.76 (m, 9H), 1.72-1.65 (m, 5H), 1.56-1.47 (m, 2H), |
| | 1.19-1.09 (m, 4H), 0.93 (d, J = 6.4 Hz, 3H), 0.80-0.70 (m, 1H) |
| 77 | ¹H NMR (400 MHz, DMSO) δ 11.07 (s, 1H), 8.29 (s, 1H), 8.18 (s, 1H), 7.96 |
| | (s, 1H), 7.91 (s, 1H), 7.90-7.86 (m, 1H), 7.64 (d, J = 8.4 Hz, 1H), 7.39 (s, |
| | 1H), 7.35 (t, J = 8.0 Hz, 1H), 7.29 (d, J = 1.6 Hz, 1H), 7.22 (dd, J = 2.0, 8.8 |
| | Hz, 1H), 6.76 (d, J = 7.6 Hz, 1H), 5.11-5.03 (m, 3H), 4.96 (d, J = 6.0 Hz, |
| | 2H), 4.88 (d, J = 6.0 Hz, 2H), 4.07 (d, J = 12.4 Hz, 2H), 3.66 (s, 2H), 3.51 (s, |
| | 2H), 2.92-2.87 (m, 6H), 2.86-2.76 (m, 6H), 2.61-2.55 (m, 2H), 2.08 (d, J = |
| | 6.4 Hz, 2H), 2.02-1.95 (m, 3H), 1.78-1.72 (m, 4H), 1.67-1.59 (m, 4H), |
| | 1.25-1.08 (m, 7H) |
| 78 | ¹H NMR (400 MHz, DMSO) δ 11.07 (s, 1H), 8.24 (s, 1H), 8.19 (s, 1H), 7.97 |
| | (s, 1H), 7.92 (s, 1H),7.89-7.86 (m, 1H), 7.63 (d, J = 7.6 Hz, 1H), 7.39-7.35 |
| | (m, 2H), 7.28-7.22 (m, 2H), 6.76 (d, J = 7.6 Hz, 1H), 5.09-4.99 (m, 3H), |
| | 4.96 (d, J = 6.0 Hz, 2H), 4.89 (d, J = 6.0 Hz, 2H), 4.04-4.01 (m, 2H), 3.66- |
| | 3.51 (m, 2H), 3.44-3.41 (m, 2H), 2.93-2.90 (m, 6H), 2.81-2.78 (m, 2H), |
| | 2.61-2.49 (m, 2H), 2.02-1.99 (m, 3H), 1.70-1.65 (m, 2H), 1.64-1.62 (m, |
| | 2H), 1.36 (s, 1H), 1.14 (s, 6H) |
| 79 | “¹H NMR (400 MHz, DMSO) δ 11.06 (s, 1H), 8.19 (s, 1H), 7.97 (s, 1H), |
| | 7.94-7.85 (m, 2H), 7.62 (d, J = 8.4 Hz, 1H), 7.43-7.31 |
| | (m, 2H), 6.81-6.71 (m, 2H), 6.61 (dd, J = 1.6, 8.4 Hz, 1H), 5.09 (s, |
| | 2H), 5.04 (dd, J = 5.2, 13.2 Hz, 1H), 4.96 (d, J = 6.0 Hz, 2H), 4.89 (d, J = 6.0 |
| | Hz, 2H), 4.19-4.11 (m, 2H), 3.70-3.58 (m, 4H), 3.51 (s, 2H), 2.90 (s, 3H), |
| | 2.87-2.76 (m, 3H), 2.61-2.54 (m, 2H), 2.04-1.91 (m, 3H), 1.66-1.53 (m, |
| | 4H), 1.38-1.10 (m, 4H).” |
| 80 | ¹H NMR (400 MHz, DMSO) δ 11.06 (s, 1H), 8.18 (s, 1H), 7.96 (d, J = 18.8 |
| | Hz, 2H), 7.87 (dd, J1 = 1.2, J2 = 8.4 Hz, 1H), 7.66 (d, J = 8.4 Hz, 1H), 7.39 (s, |
| | 1H), 7.35 (t, J = 8.0 Hz, 1H), 7.31 (d, J = 2.0 Hz, 1H), 7.23 (dd, J1 = 2.0, J1 = |
| | 8.8 Hz, 1H), 6.76 (d, J = 8.0 Hz, 1H), 5.11-5.08 (m, 2H), 5.07-5.03 (m, 1H), |
| | 4.96 (d, J = 6.0 Hz, 2H), 4.88 (d, J = 6.0 Hz, 2H), 3.84-3.74 (m, 5H), 3.51 (s, |
| | 4H), 2.89 (s, 3H), 2.88-2.82 (m, 2H), 2.60 (d, J = 2.8 Hz, 4H), 2.56-2.54 (m, |
| | 4H), 2.29 (d, J = 6.4 Hz, 2H), 2.06-1.85 (m, 3H), 1.44 (dt, J1 = 6.8, J1 = 12.4 |
| | Hz, 1H) |
| 81 | ¹H NMR (400 MHz, DMSO) δ 11.05 (s, 1 H), 8.34 (s, 2 H), 8.16 (s, 1 H), 7.94 |
| | (s, 1 H), 7.88 (s, 1 H), 7.85 (d, J = 9.6 Hz, 1 H), 7.61 (d, J = 8.8 Hz, 1 H), 7.36 (s, |
| | 1 H), 7.30-7.35 (m, 1 H), 7.28 (s, 1 H), 7.18-7.23 (m, 1 H), 6.74 (d, J = 7.6 |
| | Hz, 1 H), 5.06 (s, 2 H), 4.94 (d, J = 6.0 Hz, 2 H), 4.86 (d, J = 6.4 Hz, 2 H), 3.62 |
| | (s, 3 H), 3.46-3.50 (m, 4 H), 2.90-2.94 (m, 4 H), 2.87 (s, 3 H), 2.72-2.79 (m, |
| | 3 H), 2.55-2.59 (m, 1 H), 2.52-2.55 (m, 2 H), 2.25-2.29 (m, 2 H), 1.88- |
| | 2.01 (m, 3 H), 1.66-1.71 (m, 4 H), 1.57-1.65 (m, 2 H), 1.17-1.28 (m, 2 H), |
| | 1.06-1.17 (m, 2 H) |
| 82 | ¹H NMR (400 MHz, DMSO) δ 11.11 (s, 1 H), 8.31 (s, 2 H), 8.18 (s, 1 H), 7.97 |
| | (s, 1 H), 7.92 (s, 1 H), 7.90-7.86 (m, 1 H), 7.85-7.82 (m, 1 H), 7.80-7.74 |
| | (m, 2 H), 7.40-7.38 (m, 1 H), 7.35 (t, J = 8.0 Hz, 1 H), 6.76 (d, J = 6.8 Hz, 1 H), |
| | 5.09 (s, 2 H), 4.96 (d, J = 6.0 Hz, 2 H), 4.88 (d, J = 6.0 Hz, 2 H), 3.67 (s, 2 H), |
| | 3.55-3.47 (m, 5 H), 2.97-2.91 (m, 2 H), 2.90 (s, 3 H), 2.84-2.78 (m, 1 H), |
| | 2.20-2.10 (m, 3 H), 2.03-1.96 (m, 5 H), 1.81-1.75 (m, 3 H), 1.74-1.66 (m, |
| | 5 H), 1.57-1.47 (m, 2 H), 1.19-1.09 (m, 3 H) |
| 83 | ¹H NMR (400 MHz, DMSO) δ 11.08 (s, 1 H), 8.25 (s, 3 H), 8.19 (s, 1 H), 7.95 |
| | (s, 1 H), 7.91-7.85 (m, 2 H), 7.63 (d, J = 8.4 Hz, 1 H), 7.40-7.32 (m, 2 H), |
| | 6.78-6.73 (m, 2 H), 6.62 (dd, J = 8.4, 2.0 Hz, 1 H), 5.09 (s, 2 H), 4.96 (d, J = 6.0 |
| | Hz, 2 H), 4.88 (d, J = 6.0 Hz, 2 H), 4.10 (t, J = 8.4 Hz, 2 H), 3.75-3.69 (m, 3 H), |
| | 3.64 (s, 3 H), 3.37 (t, J = 7.2 Hz, 3 H), 3.01-2.94 (m, 3 H), 2.89 (s, 3 H), 2.77 |
| | (d, J = 10.0 Hz, 3 H), 2.54 (s, 1 H), 2.36 (d, J = 6.4 Hz, 2 H), 2.04-1.90 (m, 4 |
| | H), 1.62 (d, J = 10.8 Hz, 2 H), 1.31-1.21 (m, 2 H), 1.18-1.07 (m, 2 H). |
| 84 | ¹H NMR (400 MHz, DMSO) δ 1.10-1.18 (m, 2 H) 1.24-1.36 (m, 3 H) 1.47- |
| | 1.54 (m, 1 H) 1.65-1.77 (m, 5 H) 1.84-1.91 (m, 2 H) 1.97-2.06 (m, 3 H) |
| | 2.13 (br d, J = 7.2 Hz, 2 H) 2.62-2.63 (m, 1 H) 2.79-2.87 (m, 5 H) 2.91 (s, 3 |
| | H) 3.52-3.53 (m, 2 H) 3.67 (br s, 2 H) 4.04 (br d, J = 5.6 Hz, 2 H) 4.89 (d, |
| | J = 6.0 Hz, 2 H) 4.97 (d, J = 6.0 Hz, 2 H) 5.07-5.16 (m, 3 H) 6.77 (d, J = 8.4 Hz, |
| | 1 H) 7.33-7.38 (m, 2 H) 7.39 (s, 1 H) 7.43 (d, J = 2.0 Hz, 1 H) 7.83 (d, J = 8.4 |
| | Hz, 1 H) 7.89 (br d, J = 8.0 Hz, 1 H) 7.93 (s, 1 H) 7.98 (s, 1 H) 8.19 (s, 1 H) |
| | 8.28 (s, 2 H) 10.99-11.24 (m, 1 H) |
| 85 | ¹H NMR (400 MHz, DMSO) δ 11.07 (s, 1H), 8.17 (s, 1H), 8.13 (s, 1H), 8.00 |
| | (s, 1H), 7.95 (s, 1H), 7.84 (d, J = 8.4 Hz, 1H), 7.62 (d, J = 8.4 Hz, 1H), 7.40 (s, |
| | 1H), 7.36-7.31 (m, 1H), 7.30 (d, J = 1.2 Hz, 1H), 7.19 (dd, J = 2.0, 8.8 Hz, |
| | 1H), 6.75 (d, J = 7.6 Hz, 1H), 5.10-5.03 (m, 3H), 4.96 (dd, J = 1.2, 6.0 Hz, |
| | 2H), 4.88 (d, J = 6.0 Hz, 2H), 3.84-3.72 (m, 1H), 3.50 (s, 3H), 3.36-3.35 (m, |
| | 2H), 2.89 (s, 3H), 2.60 (d, J = 2.8 Hz, 1H), 2.56 (s, 1H), 2.54 (d, J = 1.2 Hz, |
| | 1H), 2.52 (d, J = 2.0 Hz, 4H), 2.47-2.36 (m, 5H), 2.31-2.27 (m, 2H), 2.05- |
| | 1.92 (m, 3H), 1.89-1.80 (m, 1H), 1.54-1.45 (m, 1H), 1.25-1.16 (m, 1H), |
| | 0.90 (d, J = 6.4 Hz, 3H). |
| 86 | ¹H NMR (400 MHz, DMSO) δ 11.09 (s, 1H), 8.18 (s, 1H), 7.98 (s, 1H), 7.93 |
| | (s, 1H), 7.89 (dd, J = 1.2, 8.4 Hz, 1H), 7.66 (d, J = 8.4 Hz, 1H), 7.40 (s, 1H), |
| | 7.37-7.31 (m, 2H), 7.23 (dd, J = 2.0, 8.4 Hz, 1H), 6.76 (d, J = 7.6 Hz, 1H), |
| | 5.11-5.05 (m, 3H), 4.96 (d, J = 6.0 Hz, 2H), 4.89 (d, J = 6.0 Hz, 2H), 3.76- |
| | 3.64 (m, 2H), 3.51 (s, 2H), 3.39-3.20 (m, 4H), 2.99 (d, J = 10.8 Hz, 1H), |
| | 2.90-2.85 (m, 1H), 2.81-2.73 (m, 1H), 2.62-2.56 (m, 1H), 2.55-2.52 (m, 4H), |
| | 2.46 (dd, J = 4.4, 8.0 Hz, 4H), 2.22-2.09 (m, 2H), 2.06-1.97 (m, 1H), 1.96- |
| | 1.85 (m, 1H), 1.80-1.78 (m, 1H), 1.73-1.62 (m, 1H), 1.61-1.53 (m, 2H), |
| | 0.82 (d, J = 6.4 Hz, 3H), 0.62-0.43 (m, 1H) |
| 87 | ¹H NMR (400 MHz, DMSO) δ 11.10 (s, 1H), 8.18 (s, 1H), 7.97 (s, 1H), 7.91 |
| | (s, 1H), 7.88 (dd, J = 1.2, 8.4 Hz, 1H), 7.83 (d, J = 8.4 Hz, 1H), 7.46 (d, J = 2.0 |
| | Hz, 1H), 7.40-7.32 (m, 3H), 6.76 (d, J = 7.6 Hz, 1H), 5.15-5.10 (m, 1H), |
| | 5.09 (s, 2H), 4.96 (d, J = 6.0 Hz, 2H), 4.88 (d, J = 6.0 Hz, 2H), 4.35-4.29 (m, |
| | 2H), 3.77-3.72 (m, 2H), 3.66 (s, 2H), 3.51 (s, 2H), 3.35-3.33 (m, 2H), 2.90 |
| | (s, 3H), 2.89-2.83 (m, 1H), 2.80 (d, J = 10.8 Hz, 2H), 2.63-2.54 (m, 2H), |
| | 2.07-1.94 (m, 3H), 1.68-1.60 (m, 2H), 1.59-1.49 (m, 1H), 1.25-1.14 (m, |
| | 2H). |
| 88 | ¹H NMR (400 MHz, DMSO) δ 11.08 (s, 1 H), 8.18 (s, 1 H), 8.15 (s, 2 H), 7.98 |
| | (s, 1 H), 7.93 (s, 1 H), 7.88 (dd, J = 8.4, 1.6 Hz, 1 H), 7.70 (dd, J = 8.4, 7.6 Hz, 1 |
| | H), 7.39 (s, 1 H), 7.30-7.37 (m, 3 H), 6.76 (d, J = 8.0 Hz, 1 H), 5.06-5.12 (m, |
| | 3 H), 4.96 (d, J = 6.0 Hz, 2 H), 4.89 (d, J = 6.0 Hz, 2 H), 3.69 (s, 2 H), 3.51 (s, 4 |
| | H), 3.29 (s, 6 H), 2.90 (s, 3 H), 2.77-2.87 (m, 3 H), 2.54-2.63 (m, 2 H), 2.21 |
| | (d, J = 7.2 Hz, 2 H), 2.03 (t, J = 10.4 Hz, 3 H), 1.71 (d, J = 11.2 Hz, 2 H), 1.52- |
| | 1.60 (m, 1 H), 1.16 (d, J = 10.4 Hz, 2 H). |
| 89 | ¹H NMR (400 MHz, DMSO) δ 11.11 (s, 1 H), 8.18 (s, 1 H), 7.91-8.06 (m, 2 |
| | H), 7.88 (dd, J = 8.4, 1.50 Hz, 1 H), 7.82 (d, J = 8.4 Hz, 1 H), 7.45 (d, J = 2.4 Hz, |
| | 1 H), 7.31-7.41 (m, 3 H), 6.77 (d, J = 7.6 Hz, 1 H), 5.07-5.16 (m, 3 H), 4.96 |
| | (d, J = 6.0 Hz, 2 H), 4.88 (d, J = 6.0 Hz, 2 H), 4.28-4.35 (m, 2 H), 3.77-3.80 |
| | (m, 2 H), 3.57-3.61 (m, 2 H), 3.48-3.53 (m, 4 H), 3.25 (d, J = 6.4 Hz, 3 H), |
| | 2.90 (s, 4 H), 2.83-2.87 (m, 1 H), 2.55-2.63 (m, 2 H), 2.54 (s, 1 H), 2.52 (d, |
| | J = 2.0 Hz, 2 H), 2.00-2.08 (m, 2 H), 1.60-1.70 (m, 2 H), 1.47-1.59 (m, 1 |
| | H), 1.14-1.27 (m, 2 H) |
| 90 | ¹H NMR (400 MHz, DMSO) δ 11.08 (s, 1H), 8.20-8.16 (m, 1H), 8.05-7.91 |
| | (m, 2H), 7.86 (d, J = 8.4 Hz, 1H), 7.67-7.59 (m, 1H), 7.41 (s, 1H), 7.34 (t, |
| | J = 8.0 Hz, 2H), 7.30-7.20 (m, 1H), 6.81-6.73 (m, 1H), 5.12-5.05 (m, 3H), |
| | 4.95 (d, J = 6.0 Hz, 2H), 4.88 (d, J = 5.6 Hz, 2H), 3.83-3.71 (m, 1H), 3.50 (s, |
| | 3H), 3.46-3.39 (m, 3H), 3.23-3.16 (m, 2H), 2.90 (s, 3H), 2.87 (d, J = 2.0 Hz, |
| | 1H), 2.64-2.58 (m, 1H), 2.58-2.53 (m, 3H), 2.46-2.37 (m, 3H), 2.32-2.23 |
| | (m, 2H), 2.06-1.79 (m, 4H), 1.57-1.43 (m, 1H), 1.31-1.07 (m, 2H), 0.90 (d, |
| | J = 6.0 Hz, 3H) |
| 91 | ¹H NMR (400 MHz, DMSO) δ 11.10 (s, 1 H), 8.18 (s, 1 H), 7.91-8.05 (m, 2 |
| | H), 7.88 (dd, J = 8.0, 1.6 Hz, 1 H), 7.80 (dd, J = 8.4, 7.2 Hz, 1 H), 7.53 (d, J = 8.4 |
| | Hz, 1 H), 7.45 (d, J = 7.2 Hz, 1 H), 7.33-7.41 (m, 2 H), 6.77 (d, J = 7.2 Hz, 1 |
| | H), 5.04-5.13 (m, 3 H), 4.96 (d, J = 6.0 Hz, 2 H), 4.89 (d, J = 6.0 Hz, 2 H), |
| | 4.32-4.36 (m, 2 H), 3.78-3.82 (m, 2 H), 3.62-3.65 (m, 2 H), 3.48-3.52 (m, 4 |
| | H), 3.24 (d, J = 6.0 Hz, 3 H), 2.82-2.93 (m, 5 H), 2.54-2.62 (m, 2 H), 2.52- |
| | 2.54 (m, 1 H), 2.52 (s, 2 H), 1.95-2.06 (m, 2 H), 1.48-1.70 (m, 3 H), 1.13- |
| | 1.25 (m, 2 H) |
| 92 | ¹H NMR (400 MHz, DMSO) δ 11.07 (s, 1H), 8.17 (s, 1H), 7.99 (s, 1H), 7.92 |
| | (s, 1H), 7.86 (d, J = 7.6 Hz, 1H), 7.62 (d, J = 8.8 Hz, 1H), 7.42 (s, 1H), 7.37- |
| | 7.28 (m, 1H), 7.32-7.28 (m, 1H), 7.18 (d, J = 8.8 Hz, 1H), 6.76 (d, J = 7.2 |
| | Hz, 1H), 5.11 (s, 2H), 5.08-5.04 (m, 1H), 4.95 (d, J = 5.6 Hz, 2H), 4.87 (d, |
| | J = 6.0 Hz, 2H), 3.73-3.67 (m, 1H), 3.57 (d, J = 14.0 Hz, 1H), 3.50 (s, 2H), |
| | 3.41-3.37 (m, 5H), 3.26-3.17 (m, 3H), 2.92-2.76 (m, 5H), 2.63-2.58 (m, |
| | 1H), 2.55 (d, J = 9.2 Hz, 3H), 2.45-2.37 (m, 1H), 2.29-2.17 (m, 1H), 2.05- |
| | 1.97 (m, 1H), 1.91-1.69 (m, 7H), 1.53-1.41 (m, 1H), 1.21-1.11 (m, 1H), |
| | 0.87 (d, J = 6.0 Hz, 3H) |
| 93 | ¹H NMR (400 MHz, DMSO) δ 11.07 (s, 1H), 8.18 (s, 1H), 8.01 (s, 1H), 7.94 |
| | (s, 1H), 7.87 (d, J = 8.4 Hz, 1H), 7.65 (d, J = 8.4 Hz, 1H), 7.44-7.41 (m, 1H), |
| | 7.36 (t, J = 8.0 Hz, 1H), 7.29 (s, 1H), 7.24-7.20 (m, 1H), 6.80 (d, J = 7.6 Hz, |
| | 1H), 5.11 (s, 2H), 5.10-5.09 (m, 1H), 5.06 (dd, J = 5.2, 12.4 Hz, 1H), 4.96 (d, |
| | J = 6.0 Hz, 2H), 4.88 (d, J = 6.0 Hz, 2H), 4.06-3.99 (m, 2H), 3.69 (d, J = 2.0 |
| | Hz, 2H), 3.51 (s, 2H), 3.21-3.09 (m, 2H), 2.96-2.82 (m, 7H), 2.62-2.56 (m, |
| | 1H), 2.28-2.17 (m, 3H), 2.06-1.95 (m, 2H), 1.82-1.67 (m, 7H), 1.65-1.55 |
| | (m, 3H), 1.44-1.32 (m, 2H), 1.26-1.22 (m, 1H), 1.20-1.08 (m, 6H), 0.87 (d, |
| | J = 4.0 Hz, 3H) |
| 94 | ¹H NMR (400 MHz, DMSO) δ 11.08 (s, 1 H), 8.20 (d, J = 5.6 Hz, 2 H), 7.97 (s, |
| | 1 H), 7.93 (s, 1 H), 7.89 (d, J = 9.2 Hz, 1 H), 7.64 (d, J = 8.4 Hz, 1 H), 7.40- |
| | 7.45 (m, 1 H), 7.35 (t, J = 8.0 Hz, 1 H), 7.27 (s, 1 H), 7.17-7.22 (m, 1 H), 6.77 |
| | (d, J = 7.6 Hz, 1 H), 5.10 (s, 2 H), 5.03-5.08 (m, 1 H), 4.97 (d, J = 6.0 Hz, 2 H), |
| | 4.89 (d, J = 6.0 Hz, 2 H), 3.95-4.03 (m, 2 H), 3.68-3.76 (m, 1 H), 3.48-3.53 |
| | (m, 3 H), 2.84-2.96 (m, 7 H), 2.71-2.83 (m, 2 H), 2.55-2.63 (m, 2 H), 1.93- |
| | 2.26 (m, 5 H), 1.58-1.86 (m, 8 H), 1.41-1.57 (m, 4 H), 1.00-1.17 (m, 6 |
| | H), 0.89 (d, J = 6.4 Hz, 3 H), 0.81 (d, J = 6.4 Hz, 1 H) |
| 95 | “¹H NMR (400 MHz, DMSO) δ 11.09 (s, 1H), 8.18 (s, 1H), 8.02 (s, 1H), 7.97 |
| | (s, 1H), 7.88 (d, J = 8.0 Hz, 1H), 7.64 (d, J = 8.4 Hz, 1H), 7.41 (s, 1H), 7.38- |
| | 7.32 (m, 1H), 7.29 (s, 1H), 7.22 (d, J = 8.4 Hz, 1H), 6.78 (d, J = 7.2 Hz, 1H), |
| | 5.12-5.00 (m, 3H), 4.96 (d, J = 6.0 Hz, 2H), 4.88 (d, J = 6.0 Hz, 2H), 4.03 (d, |
| | J = 12.4 Hz, 2H), 3.80-3.72 (m, 1H), 3.69-3.60 (m, 1H), 3.51 (s, 2H), 3.21- |
| | 3.03 (m, 2H), 2.97-2.84 (m, 7H), 2.63-2.55 (m, 4H), 2.41 (s, 2H), 2.18- |
| | 1.96 (m, 4H), 1.79-1.61 (m, 6H), 1.47-1.36 (m, 2H), 1.30-1.20 (m, 2H), |
| | 1.12 (d, J = 6.8 Hz, 4H), 0.96 (d, J = 6.0 Hz, |
| | 3H)” |
| 96 | ¹H NMR (400 MHz, DMSO) δ 11.08 (s, 1H), 8.19 (s, 1H), 7.99-7.92 (m, |
| | 2H), 7.90 (d, J = 8.0 Hz, 1H), 7.65 (d, J = 8.8 Hz, 1H), 7.43 (s, 1H), 7.36 (t, |
| | J = 8.0 Hz, 1H), 7.28 (s, 1H), 7.20 (d, J = 8.8 Hz, 1H), 6.78 (d, J = 8.0 Hz, 1H), |
| | 5.11 (s, 2H), 5.09-5.03 (m, 1H), 4.97 (d, J = 6.0 Hz, 2H), 4.89 (d, J = 6.0 Hz, |
| | 2H), 4.02 (d, J = 12.0 Hz, 2H), 3.76-3.60 (m, 2H), 3.52 (s, 2H), 2.92 (s, 3H), |
| | 2.89 (s, 3H), 2.80 (s, 2H), 2.66-2.55 (m, 1H), 2.48-2.41 (m, 2H), 2.20-2.10 |
| | (m, 2H), 2.07-1.99 (m, 2H), 1.92-1.75 (m, 4H), 1.74-1.63 (m, 4H), 1.63- |
| | 1.55 (m, 3H), 1.52-1.44 (m, 1H), 1.31-1.24 (m, 1H), 1.23-1.10 (m, 3H), |
| | 1.04 (d, J = 5.2 Hz, 2H), 0.97-0.88 (m, 1H) |
| 97 | ¹H NMR (400 MHz, DMSO) δ 11.09 (s, 1H), 8.20 (s, 1H), 8.18 (s, 3H), 7.98 |
| | (s, 1H), 7.93 (s, 1H), 7.89 (d, J = 8.8 Hz, 1H), 7.68 (d, J = 8.4 Hz, 1H), 7.41 (s, |
| | 1H), 7.39-7.32 (m, 2H), 7.25 (d, J = 8.4 Hz, 1H), 6.78 (d, J = 7.6 Hz, 1H), |
| | 5.18-5.06 (m, 3H), 4.98 (d, J = 6.0 Hz, 2H), 4.90 (d, J = 6.0 Hz, 2H), 3.78- |
| | 3.60 (m, 3H), 3.52 (s, 2H), 3.43 (s, 4H), 2.92 (s, 3H), 2.85 (d, J = 7.2 Hz, 4H), |
| | 2.80 (d, J = 9.6 Hz, 2H), 2.59 (d, J = 16.0 Hz, 1H), 2.55 (s, 1H), 2.47 (s, 4H), |
| | 2.24-2.13 (m, 6H), 2.11-2.02 (m, 2H), 2.01-1.87 (m, 4H), 1.84-1.73 (m, |
| | 2H), 1.71-1.59 (m, 6H), 1.57-1.45 (m, 3H), 1.20-1.09 (m, 2H), 1.08-0.99 |
| | (m, 2H), 0.98-0.88 (m, 1H) |
| 98 | ¹H NMR (400 MHz, DMSO) δ 11.08 (s, 1 H), 8.22 (s, 1 H), 8.19 (s, 1 H), 7.98 |
| | (s, 1 H), 7.93 (s, 1 H), 7.87-7.92 (m, 1 H), 7.65 (d, J = 8.4 Hz, 1 H), 7.41 (s, 1 |
| | H), 7.35 (t, J = 8.0 Hz, 1 H), 7.28 (s, 1 H), 7.14-7.24 (m, 1 H), 6.77 (d, J = 6.8 |
| | Hz, 1 H), 5.11 (s, 2 H), 5.03-5.09 (m, 1 H), 4.97 (d, J = 6.0 Hz, 2 H), 4.89 (d, |
| | J = 6.0 Hz, 2 H), 3.61-3.73 (m, 2 H), 3.52 (s, 2 H), 3.41 (d, J = 2.8 Hz, 4 H), |
| | 2.91 (s, 3 H), 2.72-2.90 (m, 2 H), 2.55-2.63 (m, 2 H), 2.44-2.48 (m, 1 H), |
| | 2.22-2.32 (m, 3 H), 1.94-2.22 (m, 5 H), 1.55-1.85 (m, 5 H), 1.45-1.54 (m, |
| | 5 H), 1.31-1.44 (m, 4 H) |
| 99 | ¹H NMR (400 MHz, DMSO) δ 11.07 (s, 1 H), 8.19 (s, 1 H), 8.14 (s, 1 H), 7.98 |
| | (s, 1 H), 7.93 (s, 1 H), 7.86-7.91 (m, 1 H), 7.52-7.59 (m, 1 H), 7.40 (s, 1 H), |
| | 7.36 (t, J = 8.0 Hz, 1 H), 7.10 (d, J = 6.8 Hz, 1 H), 6.73-6.80 (m, 2 H), 5.10 (s, 2 |
| | H), 5.04 (dd, J = 12.8, 5.6 Hz, 1 H), 4.97 (d, J = 6.0 Hz, 2 H), 4.89 (d, J = 6.0 Hz, |
| | 2 H), 4.28-4.37 (m, 2 H), 3.74-3.81 (m, 2 H), 3.69 (s, 2 H), 3.52 (s, 2 H), |
| | 2.91 (s, 3 H), 2.85-2.89 (m, 1 H), 2.82 (d, J = 10.8 Hz, 2 H), 2.77 (d, J = 6.8 Hz, |
| | 1 H), 2.53-2.62 (m, 2 H), 1.96-2.05 (m, 3 H), 1.56-1.66 (m, 4 H), 1.14- |
| | 1.28 (m, 3 H) |
| 100 | ¹H NMR (400 MHz, DMSO) δ 11.08 (s, 1 H), 8.19 (s, 1 H), 8.15 (s, 1 H), 7.99 |
| | (s, 1 H), 7.94 (s, 1 H), 7.89 (d, J = 8.0 Hz, 1 H), 7.66 (d, J = 8.4 Hz, 1 H), 7.40 (s, |
| | 1 H), 7.36 (t, J = 8.0 Hz, 1 H), 7.32 (s, 1 H), 7.20-7.26 (m, 1 H), 6.79 (d, J = 7.6 |
| | Hz, 1 H), 5.11 (s, 2 H), 5.04-5.09 (m, 1 H), 4.97 (d, J = 6.0 Hz, 2 H), 4.89 (d, |
| | J = 6.0 Hz, 2 H), 3.71 (s, 2 H), 3.52 (s, 2 H), 3.47 (s, 4 H), 2.92 (s, 3 H), 2.82- |
| | 2.90 (m, 5 H), 2.70-2.78 (m, 1 H), 2.55-2.63 (m, 2 H), 2.55 (s, 2 H), 2.00- |
| | 2.09 (m, 3 H), 1.85-1.94 (m, 2 H), 1.51-1.64 (m, 10 H) |
| 101 | ¹H NMR (400 MHz, DMSO) δ 11.08 (s, 1 H), 8.19 (s, 1 H), 8.17 (s, 2 H), 7.98 |
| | (s, 1 H), 7.93 (s, 1 H), 7.87-7.91 (m, 1 H), 7.68 (d, J = 8.4 Hz, 1 H), 7.41 (s, 1 |
| | H), 7.33-7.38 (m, 1 H), 7.30-7.33 (m, 1 H), 7.24 (d, J = 8.8 Hz, 1 H), 6.77 (d, |
| | J = 7.6 Hz, 1 H), 5.11 (s, 2 H), 5.03-5.09 (m, 1 H), 4.97 (d, J = 6.0 Hz, 2 H), |
| | 4.89 (d, J = 6.0 Hz, 2 H), 3.61-3.74 (m, 3 H), 3.52 (s, 2 H), 3.40 (s, 2 H), 2.92 |
| | (s, 3 H), 2.82-2.89 (m, 2 H), 2.79 (d, J = 1.2 Hz, 2 H), 2.70 (d, J = 1.6 Hz, 1 H), |
| | 2.55-2.63 (m, 2 H), 2.55 (s, 1 H), 2.53 (s, 2 H), 2.43 (s, 3 H), 2.18 (dd, J = 8.8, |
| | 3.2 Hz, 1 H), 2.12 (d, J = 7.2 Hz, 2 H), 2.03 (dd, J = 6.0, 4.0 Hz, 2 H), 1.84-1.95 |
| | (m, 2 H), 1.72-1.83 (m, 2 H), 1.58-1.70 (m, 4 H), 1.45-1.54 (m, 2 H), |
| | 1.00-1.11 (m, 2 H), 0.89-0.96 (m, 1 H) |
| 102 | ¹H NMR (400 MHz, DMSO) δ 11.11 (s, 1H), 8.19 (s, 1H), 8.19 (s, 1H), 7.97 |
| | (s, 1H), 7.92 (s, 1H), 7.88 (d, J = 9.2 Hz, 1H), 7.83 (d, J = 8.4 Hz, 1H), 7.45 |
| | (d, J = 2.0 Hz, 1H), 7.40 (s, 1H), 7.38-7.32 (m, 2H), 6.76 (d, J = 8.0 Hz, 1H), |
| | 5.16-5.07 (m, 3H), 4.97 (d, J = 6.0 Hz, 2H), 4.89 (d, J = 6.0 Hz, 2H), 4.36- |
| | 4.26 (m, 2H), 3.78-3.70 (m, 2H), 3.69 (s, 2H), 3.52 (s, 2H), 3.28 (d, J = 6.4 |
| | Hz, 4H), 2.90 (s, 3H), 2.88-2.84 (m, 1H), 2.80 (d, J = 10.0 Hz, 2H), 2.71 (d, |
| | J = 8.0 Hz, 1H), 2.64-2.56 (m, 2H), 2.23-2.13 (m, 2H), 2.08-2.02 (m, 1H), |
| | 1.88-1.77 (m, 2H), 1.60 (d, J = 11.2 Hz, 3H), 1.56-1.45 (m, 2H), 1.13-1.01 |
| | (m, 2H), 0.92-0.86 (m, 1H), 0.84 (d, J = 6.4 Hz, 3H) |
| 103 | ¹H NMR (400 MHz, DMSO) δ 1.01-1.21 (m, 2 H) 1.35-1.51 (m, 3 H) 1.67- |
| | 1.86 (m, 5 H) 1.96-2.04 (m, 3 H) 2.12 (d, J = 6.4 Hz, 2 H) 2.16-2.21 (m, 1 H) |
| | 2.41 (d, J = 9.2 Hz, 4 H) 2.58 (d, J = 16.8 Hz, 1 H) 2.86 (dd, J = 9.2, 5.6 Hz, 3 H) |
| | 2.91 (s, 3 H) 2.92-3.01 (m, 2 H) 3.34 (s, 4 H) 3.52 (s, 2 H) 3.67 (s, 2 H) 4.03 |
| | (d, J = 13.2 Hz, 2 H) 4.89 (d, J = 6.0 Hz, 2 H) 4.97 (d, J = 6.0 Hz, 2 H) 5.03-5.13 |
| | (m, 3 H) 6.77 (d, J = 7.2 Hz, 1 H) 7.17-7.25 (m, 1 H) 7.30 (s, 1 H) 7.36 (t, |
| | J = 8.0 Hz, 1 H) 7.40 (s, 1 H) 7.65 (d, J = 8.4 Hz, 1 H) 7.89 (d, J = 8.2 Hz, 1 H) |
| | 7.91-8.00 (m, 2 H) 8.17 (s, 1 H) 8.19 (s, 1 H) 10.55-11.41 (m, 1 H) |
| 104 | ¹H NMR (400 MHz, DMSO) δ 1.06-1.18 (m, 2 H) 1.35-1.46 (m, 2 H) 1.68- |
| | 1.82 (m, 5 H) 1.88 (t, J = 11.2 Hz, 2 H) 1.97-2.04 (m, 1 H) 2.13 (d, J = 6.4 Hz, |
| | 2 H) 2.16-2.23 (m, 1 H) 2.40-2.45 (m, 4 H) 2.53 (s, 4 H) 2.55-2.63 (m, 2 |
| | H) 2.82-2.90 (m, 3 H) 2.91 (s, 3 H) 2.92-3.00 (m, 2 H) 3.52 (s, 2 H) 3.68 (s, |
| | 2 H) 4.03 (d, J = 12.8 Hz, 2 H) 4.89 (d, J = 6.0 Hz, 2 H) 4.97 (d, J = 6.0 Hz, 2 H) |
| | 5.04-5.13 (m, 3 H) 6.77 (d, J = 7.6 Hz, 1 H) 7.22 (dd, J = 8.8, 1.6 Hz, 1 H) 7.30 |
| | (s, 1 H) 7.36 (t, J = 8.0 Hz, 1 H) 7.40 (s, 1 H) 7.65 (d, J = 8.8 Hz, 1 H) 7.86- |
| | 7.91 (m, 1 H) 7.91-8.02 (m, 2 H) 8.17 (s, 1 H) 8.19 (s, 1 H) 11.08 (s, 1 H) |
| 105 | ¹H NMR (400 MHz, DMSO) δ 11.09 (s, 1H), 8.19 (s, 1H), 8.03 (s, 1H), 7.97 |
| | (s, 1H), 7.88 (d, J = 8.8 Hz, 1H), 7.66 (d, J = 8.0 Hz, 1H), 7.41-7.33 (m, 2H), |
| | 7.31 (s, 1H), 7.22 (d, J = 8.8 Hz, 1H), 6.80 (d, J = 8.0 Hz, 1H), 5.15-5.03 (m, |
| | 3H), 4.96 (d, J = 6.0 Hz, 2H), 4.89 (d, J = 6.0 Hz, 2H), 3.84-3.73 (m, 2H), |
| | 3.51 (s, 2H), 3.46 (d, J = 1.2 Hz, 5H), 3.12-3.06 (m, 1H), 2.91 (s, 3H), 2.90- |
| | 2.82 (m, 2H), 2.60 (d, J = 1.6 Hz, 1H), 2.58-2.53 (m, 3H), 2.01 (d, J = 5.6 |
| | Hz, 2H), 1.87-1.76 (m, 4H), 1.60-1.47 (m, 4H), 1.41 (s, 3H), 1.29-0.97 (m, |
| | 4H |
| 106 | ¹H NMR (400 MHz, DMSO) δ 11.19-10.96 (m, 1H), 8.19 (s, 1H), 8.16 (d, |
| | J = 1.6 Hz, 1H), 7.97 (s, 1H), 7.94-7.87 (m, 2H), 7.64 (d, J = 8.0 Hz, 1H), |
| | 7.42-7.32 (m, 2H), 6.81-6.74 (m, 2H), 6.67-6.58 (m, 1H), 5.11-5.08 (m, 2H), |
| | 5.07-5.03 (m, 1H), 4.96 (d, J = 6.0 Hz, 2H), 4.89 (d, J = 6.0 Hz, 2H), 4.10 (s, |
| | 4H), 3.68 (s, 3H), 3.51 (s, 2H), 2.90 (s, 3H), 2.88 (s, 2H), 2.73 (d, J = 10.4 Hz, |
| | 2H), 2.59 (d, J = 2.0 Hz, 1H), 2.46-2.44 (m, 3H), 2.08-1.98 (m, 4H), 1.62 |
| | (d, J = 11.2 Hz, 2H), 1.23-1.13 (m, 2H) |
| 107 | ¹H NMR (400 MHz, DMSO) δ 11.07 (s, 1 H), 8.19 (s, 1 H), 7.97-8.03 (m, 1 |
| | H), 7.93-7.97 (m, 1 H), 7.89 (d, J = 9.6 Hz, 1 H), 7.62-7.66 (m, 1 H), 7.40 (s, |
| | 1 H), 7.36 (t, J = 8.0 Hz, 1 H), 6.74-6.81 (m, 2 H), 6.63 (d, J = 7.6 Hz, 1 H), |
| | 5.11 (s, 2 H), 5.03-5.09 (m, 1 H), 4.97 (d, J = 6.4 Hz, 2 H), 4.89 (d, J = 6.0 Hz, |
| | 2 H), 4.07 (s, 2 H), 3.92-4.00 (m, 2 H), 3.64-3.81 (m, 2 H), 3.52 (s, 2 H), |
| | 2.92 (s, 3 H), 2.79-2.91 (m, 1 H), 2.59-2.63 (m, 2 H), 2.54-2.55 (m, 4 H), |
| | 2.38-2.45 (m, 6 H), 1.96-2.08 (m, 4 H) |
| 108 | ¹H NMR (400 MHz, DMSO) δ 11.07 (s, 1 H), 8.20 (s, 1 H), 8.18 (s, 1 H), 7.98 |
| | (s, 1 H), 7.93 (s, 1 H), 7.88 (d, J = 9.2 Hz, 1 H), 7.64 (d, J = 8.8 Hz, 1 H), 7.39 (s, |
| | 1 H), 7.33-7.37 (m, 1 H), 7.31 (s, 1 H), 7.23 (d, J = 8.8 Hz, 1 H), 6.76 (d, |
| | J = 8.0 Hz, 1 H), 5.10 (s, 2 H), 5.06 (dd, J = 12.8, 5.6 Hz, 1 H), 4.96 (d, J = 6.0 |
| | Hz, 2 H), 4.88 (d, J = 6.0 Hz, 2 H), 3.51 (s, 2 H), 3.42-3.43 (m, 4 H), 2.97 (s, 4 |
| | H), 2.90 (s, 3 H), 2.86 (d, J = 11.6 Hz, 2 H), 2.71-2.76 (m, 1 H), 2.55-2.61 |
| | (m, 2 H), 2.52-2.53 (m, 2 H), 2.25-2.32 (m, 1 H), 1.94-2.03 (m, 2 H), 1.72 |
| | (s, 6 H), 1.61-1.67 (m, 1 H), 1.37-1.47 (m, 1 H), 0.83-0.98 (m, 1 H) |
| 109 | ¹H NMR (400 MHz, DMSO) δ 11.08 (s, 1 H), 8.20 (s, 1 H), 8.15 (s, 1 H), 8.03 |
| | (s, 1 H), 7.96 (s, 1 H), 7.88 (d, J = 8.4 Hz, 1 H), 7.70 (d, J = 8.4 Hz, 1 H), 7.42 |
| | (d, J = 1.6 Hz, 1 H), 7.37 (s, 1 H), 7.24-7.32 (m, 2 H), 6.82 (d, J = 8.4 Hz, 1 H), |
| | 5.13 (s, 2 H), 5.08 (d, J = 7.6 Hz, 1 H), 4.98 (d, J = 6.0 Hz, 2 H), 4.90 (d, J = 6.4 |
| | Hz, 2 H), 3.72-3.82 (m, 2 H), 3.53 (s, 2 H), 3.45 (s, 4 H), 2.98-3.06 (m, 2 |
| | H), 2.94 (s, 3 H), 2.84-2.91 (m, 2 H), 2.72-2.83 (m, 4 H), 2.63-2.65 (m, 2 |
| | H), 2.61 (s, 4 H), 2.45 (d, J = 7.2 Hz, 2 H), 2.18-2.25 (m, 3 H), 2.01-2.07 (m, |
| | 2 H), 1.90-1.98 (m, 2 H), 1.74-1.87 (m, 7 H), 1.69-1.74 (m, 1 H), 1.40- |
| | 1.56 (m, 3 H), 1.23-1.37 (m, 4 H). |
| 110 | ¹H NMR (400 MHz, DMSO) δ 11.09 (s, 1H), 8.19 (s, 1H), 8.18 (s, 1H), 7.98 |
| | (s, 1H), 7.92 (s, 1H), 7.92-7.86 (m, 1H), 7.64 (d, J = 8.8 Hz, 1H), 7.41 (s, |
| | 1H), 7.35 (t, J = 8.0 Hz, 1H), 7.28 (s, 1H), 7.24-7.18 (m, 1H), 6.77 (d, J = 7.6 |
| | Hz, 1H), 5.12-5.08 (m, 2H), 5.07 (d, J = 7.6 Hz, 1H), 4.97 (d, J = 6.0 Hz, |
| | 2H), 4.89 (d, J = 6.0 Hz, 2H), 4.01 (d, J = 13.6 Hz, 2H), 3.73-3.61 (m, 3H), |
| | 3.51 (s, 3H), 2.95-2.86 (m, 6H), 2.85-2.74 (m, 3H), 2.62-2.56 (m, 1H), |
| | 2.18-2.09 (m, 2H), 2.06-1.98 (m, 2H), 1.91-1.84 (m, 1H), 1.78 (dd, J = 2.0, |
| | 9.2 Hz, 3H), 1.67 (d, J = 13.2 Hz, 4H), 1.57 (d, J = 12.8 Hz, 4H), 1.48 (dd, J = |
| | 2.0, 7.6 Hz, 1H), 1.33-1.26 (m, 1H), 1.12-1.04 (m, 4H), 1.02-0.92 (m, 2H) |
| 111 | ¹H NMR (400 MHz, DMSO) δ 11.08 (s, 1H), 8.21 (s, 1H), 7.96 (s, 1H), 7.94- |
| | 7.86 (m, 2H), 7.63 (d, J = 8.4 Hz, 1H), 7.40 (s, 1H), 7.34 (t, J = 8.0 Hz, 1H), |
| | 7.27 (s, 1H), 7.20 (d, J = 9.2 Hz, 1H), 6.76 (d, J = 8.0 Hz, 1H), 5.09 (s, 2H), |
| | 5.08-5.02 (m, 1H), 4.96 (d, J = 6.0 Hz, 2H), 4.88 (d, J = 6.0 Hz, 2H), 4.00 (d, |
| | J = 12.0 Hz, 2H), 3.66 (d, J = 17.2 Hz, 2H), 3.51 (s, 2H), 2.92 (s, 1H), 2.90 (s, |
| | 3H), 2.87 (s, 1H), 2.79 (d, J = 8.4 Hz, 2H), 2.73-2.69 (m, 1H), 2.63-2.56 (m, |
| | 2H), 2.15-2.07 (m, 2H), 2.04-1.97 (m, 2H), 1.87-1.78 (m, 2H), 1.74 (dd, |
| | J = 1.6, 12.0 Hz, 2H), 1.70-1.61 (m, 4H), 1.61-1.51 (m, 4H), 1.50-1.42 (m, |
| | 1H), 1.31-1.23 (m, 1H), 1.13 (d, J = 7.2 Hz, 1H), 1.10-1.02 (m, 4H), 1.01- |
| | 0.80 (m, 3H) |
| 112 | ¹H NMR (400 MHz, DMSO) δ 10.95 (s, 1H), 8.19 (s, 1H), 8.15 (s, 1H), 7.97 |
| | (s, 1H), 7.92 (s, 1H), 7.88 (dd, J = 1.2, 8.0 Hz, 1H), 7.74 (d, J = 8.8 Hz, 1H), |
| | 7.42-7.31 (m, 2H), 6.91 (d, J = 8.8 Hz, 1H), 6.76 (d, J = 7.6 Hz, 1H), 5.12- |
| | 5.03 (m, 3H), 4.96 (d, J = 6.0 Hz, 2H), 4.88 (d, J = 6.0 Hz, 2H), 4.25 (d, J = |
| | 17.6 Hz, 1H), 4.12-4.02 (m, 3H), 3.68 (s, 3H), 3.51 (s, 2H), 2.90 (s, 3H), 2.59 |
| | (d, J = 2.0 Hz, 2H), 2.43-2.34 (m, 7H), 2.07 (d, J = 7.2 Hz, 3H), 2.01-1.68 |
| | (m, 10H), 1.50-1.31 (m, 4H), 1.17-1.08 (m, 2H), 0.91-0.81 (m, 2H) |
| 113 | ¹H NMR (400 MHz, DMSO) δ 10.98 (s, 1H), 8.19 (s, 1H), 8.05-7.93 (m, |
| | 2H), 7.92-7.86 (m, 1H), 7.47 (d, J = 8.0 Hz, 1H), 7.40 (s, 1H), 7.36 (t, J = 8.0 |
| | Hz, 1H), 7.17 (t, J = 8.0 Hz, 1H), 6.77 (d, J = 7.6 Hz, 1H), 5.11 (s, 2H), 5.10- |
| | 5.04 (m, 1H), 4.97 (d, J = 6.0 Hz, 2H), 4.90 (d, J = 6.0 Hz, 2H), 4.53-4.27 |
| | (m, 2H), 3.82-3.58 (m, 3H), 3.52 (s, 2H), 3.39 (s, 3H), 3.38 (s, 4H), 3.01- |
| | 2.93 (m, 2H), 2.91 (s, 3H), 2.89-2.83 (m, 1H), 2.78-2.69 (m, 1H), 2.64- |
| | 2.58 (m, 1H), 2.42 (dd, J = 4.4, 12.8 Hz, 1H), 2.38-2.23 (m, 4H), 2.23-2.04 |
| | (m, 4H), 1.99-1.88 (m, 5H), 1.83 (s, 2H), 1.76-1.44 (m, 8H), 1.44-1.33 (m, |
| | 2H), 1.29-1.19 (m, 2H), 1.03-0.89 (m, 1H) |
| 114 | ¹H NMR (400 MHz, DMSO) δ 11.08 (s, 1 H), 8.20 (s, 1 H), 8.18 (s, 2 H), 7.98 |
| | (s, 1 H), 7.93 (s, 1 H), 7.89 (dd, J = 8.4, 1.6 Hz, 1 H), 7.64 (d, J = 8.4 Hz, 1 H), |
| | 7.42 (s, 1 H), 7.35 (t, J = 8.0 Hz, 1 H), 6.76-6.79 (m, 2 H), 6.64 (dd, J = 8.4, 1.6 |
| | Hz, 1 H), 5.11 (s, 2 H), 5.06 (dd, J = 13.2, 5.2 Hz, 1 H), 4.97 (d, J = 6.0 Hz, 2 H) |
| | 4.89 (d, J = 6.00 Hz, 2 H) 4.08 (s, 4 H) 3.70 (br s, 1 H) 3.52 (s, 2 H) 3.33 (br s, |
| | 2 H) 2.92 (s, 3 H) 2.78-2.86 (m, 3 H) 2.71-2.78 (m, 2 H) 2.57 (br s, 3 H) |
| | 2.25 (br d, J = 5.6 Hz, 2 H) 2.14 (br s, 2 H) 1.99-2.06 (m, 2 H) 1.71-1.89 (m, |
| | 5 H) 1.64 (br s, 2 H) 1.58 (br d, J = 12.8 Hz, 3 H) 1.45-1.52 (m, 1 H) 1.18- |
| | 1.25 (m, 1 H) 0.98-1.07 (m, 2 H) 0.88-0.95 (m, 1 H) |
| 115 | ¹H NMR (400 MHz, DMSO) δ 11.08 (s, 1 H), 8.19 (s, 1 H), 7.85-7.91 (m, 1 |
| | H), 7.66 (d, J = 8.4 Hz, 1 H), 7.30-7.37 (m, 3 H), 7.24 (dd, J = 8.4, 1.6 Hz, 1 H), |
| | 6.94 (d, J = 2.8 Hz, 2 H), 6.75 (d, J = 8.0 Hz, 1 H), 5.07 (dd, J = 12.8, 5.6 Hz, 1 |
| | H), 4.93-4.98 (m, 4 H), 4.89 (d, J = 6.0 Hz, 2 H), 4.05 (d, J = 12.0 Hz, 2 H), |
| | 3.70 (s, 4 H), 3.51 (s, 2 H), 2.97 (s, 2 H), 2.90 (s, 3 H), 2.55-2.63 (m, 2 H), |
| | 2.36-2.46 (m, 4 H), 2.14-2.25 (m, 2 H), 1.99-2.05 (m, 1 H), 1.79 (s, 7 H), |
| | 1.10-1.28 (m, 3 H). |
| 116 | ¹H NMR (400 MHz, DMSO) δ 11.12 (s, 1 H), 8.20 (s, 1 H), 7.89 (dd, J = 8.4, |
| | 1.2 Hz, 1 H), 7.74 (d, J = 11.6 Hz, 1 H), 7.50 (d, J = 7.6 Hz, 1 H), 7.32-7.38 (m, |
| | 2 H), 6.97 (d, J = 3.2 Hz, 2 H), 6.76 (d, J = 7.6 Hz, 1 H), 5.12 (dd, J = 12.8, 5.2 |
| | Hz, 1 H), 4.95-4.99 (m, 4 H), 4.89 (d, J = 6.0 Hz, 2 H), 3.81 (s, 4 H), 3.52 (s, 2 |
| | H), 3.28 (s, 3 H), 2.91 (s, 3 H), 2.55-2.65 (m, 2 H), 1.98-2.10 (m, 2 H), 1.96 |
| | (s, 4 H), 1.24-1.28 (m, 1 H) |
| 117 | ¹H NMR (400 MHz, DMSO) δ 10.95 (s, 1H), 8.19 (s, 1H), 7.88 (d, J = 8.4 Hz, |
| | 1H), 7.51 (d, J = 8.4 Hz, 1H), 7.39-7.32 (m, 2H), 7.12-7.02 (m, 2H), 6.94 |
| | (d, J = 6.4 Hz, 2H), 6.76 (d, J = 7.6 Hz, 1H), 5.05 (dd, J = 5.2, 13.2 Hz, 1H), |
| | 4.99-4.93 (m, 4H), 4.89 (d, J = 6.0 Hz, 2H), 4.35-4.17 (m, 2H), 3.90 (d, J = |
| | 12.0 Hz, 2H), 3.74 (s, 4H), 3.51 (s, 2H), 3.48-3.42 (m, 1H), 3.31-3.31 (m, |
| | 1H), 2.95 (d, J = 5.2 Hz, 1H), 2.91 (s, 3H), 2.89-2.78 (m, 3H), 2.70-2.59 (m, |
| | 2H), 2.59-2.54 (m, 2H), 2.41-2.33 (m, 2H), 1.96 (dd, J = 5.2, 10.0 Hz, 2H), |
| | 1.90 (d, J = 6.0 Hz, 2H), 1.82 (d, J = 12.4 Hz, 2H), 1.23 (d, J = 9.2 Hz, 2H), |
| | 1.06 (t, J = 6.8 Hz, 1H) |
| 118 | ¹H NMR (400 MHz, DMSO) δ 11.21-11.01 (m, 1H), 8.18 (s, 1H), 7.88 (dd, J = |
| | 1.5, 8.0 Hz, 1H), 7.70 (d, J = 11.6 Hz, 1H), 7.49 (s, 1H), 7.46-7.41 (m, 2H), |
| | 7.37-7.31 (m, 2H), 6.75 (d, J = 7.6 Hz, 1H), 5.10 (dd, J = 5.2, 12.8 Hz, 1H), |
| | 4.98-4.93 (m, 4H), 4.88 (d, J = 6.0 Hz, 2H), 3.89 (d, J = 12.4 Hz, 2H), 3.66- |
| | 3.56 (m, 2H), 3.51 (s, 2H), 3.29-3.28 (m, 1H), 2.95-2.80 (m, 8H), 2.57 (s, |
| | 1H), 2.05 (td, J = 5.2, 10.8 Hz, 1H), 1.79 (d, J = 12.4 Hz, 4H), 1.62 (s, 2H), |
| | 1.33-1.19 (m, 6H) |
| 119 | ¹H NMR (400 MHz, DMSO) δ 10.99 (s, 1H), 8.19 (s, 1H), 8.17 (s, 1H), 7.88 |
| | (d, J = 8.8 Hz, 1H), 7.46-7.40 (m, 1H), 7.37-7.33 (m, 2H), 7.30 (d, J = 7.2 |
| | Hz, 1H), 7.16 (d, J = 8.0 Hz, 1H), 6.94 (d, J = 1.6 Hz, 2H), 6.75 (d, J = 7.6 Hz, |
| | 1H), 5.12 (dd, J = 5.2, 13.2 Hz, 1H), 4.97-4.93 (m, 4H), 4.88 (d, J = 6.0 Hz, |
| | 2H), 4.47-4.39 (m, 1H), 4.32-4.26 (m, 1H), 3.70 (s, 4H), 3.51 (s, 3H), 3.40- |
| | 3.38 (m, 2H), 2.90 (s, 4H), 2.78-2.66 (m, 3H), 2.58-2.54 (m, 1H), 2.36 (s, |
| | 2H), 2.19 (d, J = 6.8 Hz, 2H), 2.02-1.97 (m, 1H), 1.85-1.70 (m, 8H), 1.30- |
| | 1.22 (m, 2H) |
| 120 | ¹H NMR (400 MHz, DMSO) δ 10.97-11.28 (m, 1 H), 8.19 (s, 1 H), 7.89 (d, |
| | J = 8.0 Hz, 1 H), 7.65 (d, J = 8.0 Hz, 1 H), 7.31-7.38 (m, 2 H), 6.94 (d, J = 5.6 |
| | Hz, 2 H), 6.73-6.80 (m, 2 H), 6.65 (dd, J = 8.4, 2.0 Hz, 1 H), 5.06 (dd, J = 13.2, |
| | 5.2 Hz, 1 H), 4.93-4.98 (m, 4 H), 4.89 (d, J = 6.0 Hz, 2 H), 4.17 (t, J = 8.0 Hz, 2 |
| | H), 4.11 (t, J = 7.6 Hz, 2 H), 3.73 (dd, J = 7.6, 6.4 Hz, 2 H), 3.64 (t, J = 6.8 Hz, 2 |
| | H), 3.51 (s, 2 H), 2.90 (s, 3 H), 2.79-2.85 (m, 2 H), 2.60 (s, 2 H), 2.55 (s, 2 |
| | H), 2.02 (d, J = 7.2 Hz, 2 H) |
| 121 | ¹H NMR (400 MHz, DMSO) δ 11.04-11.11 (m, 1 H), 8.19 (d, J = 1.6 Hz, 1 |
| | H), 7.89 (d, J = 8.0 Hz, 1 H), 7.61-7.70 (m, 1 H), 7.32-7.37 (m, 2 H), 6.97 (s, |
| | 2 H), 6.82 (s, 1 H), 6.76 (d, J = 7.6 Hz, 1 H), 6.68 (d, J = 8.4 Hz, 1 H), 5.03- |
| | 5.10 (m, 1 H), 4.94-4.98 (m, 4 H), 4.89 (d, J = 4.8 Hz, 2 H), 4.19 (t, J = 7.2 Hz, |
| | 2 H), 4.12 (t, J = 7.2 Hz, 2 H), 3.78-3.84 (m, 2 H), 3.75 (d, J = 5.6 Hz, 2 H), |
| | 3.51 (s, 2 H), 3.11-3.20 (m, 2 H), 2.91 (d, J = 1.6 Hz, 3 H), 2.61 (s, 1 H), 2.55 |
| | (d, J = 2.0 Hz, 2 H), 1.97-2.06 (m, 1 H) |
| 122 | ¹H NMR (400 MHz, DMSO) δ 11.09 (s, 1H), 8.20 (s, 1H), 7.92-7.87 (m, |
| | 1H), 7.67 (d, J = 8.8 Hz, 1H), 7.59-7.44 (m, 2H), 7.38-7.33 (m, 3H), 7.26 |
| | (d, J = 8.0 Hz, 1H), 6.77 (d, J = 7.2 Hz, 1H), 5.08 (dd, J = 5.2, 13.2 Hz, 1H), |
| | 4.99 (s, 2H), 4.97 (d, J = 6.0 Hz, 2H), 4.89 (d, J = 6.0 Hz, 2H), 4.12-4.04 (m, |
| | 2H), 3.52 (s, 2H), 3.33 (s, 8H), 3.05-2.98 (m, 2H), 2.92 (s, 2H), 2.86 (d, J = |
| | 5.6 Hz, 2H), 2.61 (d, J = 2.4 Hz, 2H), 2.58-2.32 (m 2H), 2.27-2.19 (m, 1H), |
| | 2.05-2.00 (m, 1H), 1.88-1.82 (m, 2H), 1.30-1.16 (m, 2H) |
| 123 | ¹H NMR (400 MHz, DMSO) δ 11.09 (s, 1 H), 8.33-8.43 (m, 1 H), 7.90 (d, |
| | J = 9.6 Hz, 1 H), 7.69 (d, J = 8.4 Hz, 1 H), 7.33-7.39 (m, 2 H), 7.01 (s, 2 H), |
| | 6.86 (d, J = 1.6 Hz, 1 H), 6.79 (d, J = 7.6 Hz, 1 H), 6.72 (dd, J = 8.0, 1.6 Hz, 1 H), |
| | 5.08 (dd, J = 13.2, 5.2 Hz, 1 H), 4.95-4.99 (m, 4 H), 4.90 (d, J = 6.0 Hz, 2 H), |
| | 4.28 (s, 4 H), 4.22 (s, 4 H), 3.56 (s, 2 H), 2.95 (s, 3 H), 2.86-2.90 (m, 2 H), |
| | 2.55 (s, 1 H), 1.99-2.06 (m, 1 H) |
| 124 | ¹H NMR (400 MHz, DMSO) δ 11.08 (s, 1 H), 8.19 (s, 1 H), 7.89 (dd, J = 8.0, |
| | 1.6 Hz, 1 H), 7.66 (d, J = 8.4 Hz, 1 H), 7.49 (d, J = 1.6 Hz, 1 H), 7.43 (s, 1 H), |
| | 7.33-7.37 (m, 2 H), 7.32 (s, 1 H), 7.25 (dd, J = 8.8, 2.0 Hz, 1 H), 6.75 (d, J = 8.0 |
| | Hz, 1 H), 5.07 (dd, J = 12.8, 5.6 Hz, 1 H), 4.94-4.99 (m, 4 H), 4.89 (d, J = 6.0 |
| | Hz, 2 H), 4.11 (d, J = 12.4 Hz, 2 H), 3.94 (d, J = 11.6 Hz, 2 H), 3.51 (s, 2 H), |
| | 2.92 (s, 1 H), 2.90 (s, 3 H), 2.73-2.82 (m, 2 H), 2.57-2.62 (m, 1 H), 2.55 (s, |
| | 2 H), 2.53 (d, J = 2.0 Hz, 1 H), 1.97-2.05 (m, 1 H), 1.81 (d, J = 9.2 Hz, 4 H), |
| | 1.37-1.45 (m, 1 H), 1.22-1.35 (m, 5 H) |
| 125 | ¹H NMR (400 MHz, DMSO) δ 11.08 (s, 1H), 8.19 (s, 1H), 7.90 (dd, J = 1.6, |
| | 8.0 Hz, 1H), 7.67 (d, J = 8.4 Hz, 1H), 7.48 (d, J = 18.4 Hz, 2H), 7.38-7.32 |
| | (m, 3H), 7.26 (dd, J = 2.0, 8.4 Hz, 1H), 6.76 (d, J = 7.6 Hz, 1H), 5.08 (dd, J = |
| | 5.6, 12.8 Hz, 1H), 4.97 (d, J = 5.6 Hz, 4H), 4.89 (d, J = 6.0 Hz, 2H), 3.52 (s, |
| | 6H), 3.37 (d, J = 5.2 Hz, 4H), 2.91 (s, 3H), 2.61 (d, J = 4.8 Hz, 1H), 2.58- |
| | 2.54 (m, 2H), 2.04 (s, 1H), 1.68-1.59 (m, 8H) |
| 126 | ¹H NMR (400 MHz, DMSO) δ 11.08 (s, 1H), 8.20 (s, 1H), 7.89 (d, J = 9.2 Hz, |
| | 1H), 7.68 (d, J = 8.4 Hz, 1H), 7.39-7.31 (m, 2H), 7.00 (d, J = 6.0 Hz, 2H), |
| | 6.96 (s, 1H), 6.86 (d, J = 8.4 Hz, 1H), 6.76 (d, J = 7.6 Hz, 1H), 5.07 (dd, J = |
| | 5.2, 12.8 Hz, 1H), 4.97 (d, J = 5.6 Hz, 4H), 4.89 (d, J = 6.0 Hz, 2H), 4.02 (s, |
| | 4H), 3.72 (s, 2H), 3.57-3.50 (m, 4H), 2.91 (s, 3H), 2.61 (d, J = 2.8 Hz, 1H), |
| | 2.57-2.54 (m, 2H), 2.34 (t, J = 6.4 Hz, 2H), 2.06-1.99 (m, 1H) |
| 127 | ¹H NMR (400 MHz, DMSO) δ 11.08 (s, 1 H), 8.20 (s, 1 H), 7.90 (dd, J = 8.0, |
| | 1.6 Hz, 1 H), 7.68 (d, J = 8.4 Hz, 1 H), 7.32-7.38 (m, 2 H), 7.06 (s, 2 H), 6.85 |
| | (d, J = 2.0 Hz, 1 H), 6.75 (d, J = 7.6 Hz, 1 H), 6.71 (dd, J = 8.4, 2.0 Hz, 1 H), 5.07 |
| | (dd, J = 12.8, 5.2 Hz, 1 H), 4.94-4.98 (m, 4 H), 4.89 (d, J = 6.0 Hz, 2 H), 4.05- |
| | 4.12 (m, 4 H), 3.69 (s, 2 H), 3.47-3.53 (m, 4 H), 2.91 (s, 3 H), 2.83-2.90 (m, |
| | 1 H), 2.58-2.63 (m, 1 H), 2.55 (s, 1 H), 2.53 (s, 1 H), 2.34-2.36 (m, 1 H), |
| | 1.99-2.06 (m, 1 H) |
| 128 | ¹H NMR (400 MHz, DMSO) δ 11.08 (d, J = 2.0 Hz, 1 H), 8.19 (s, 1 H), 7.90 |
| | (dd, J = 8.0, 1.6 Hz, 1 H), 7.66 (d, J = 8.4 Hz, 1 H), 7.51 (s, 1 H), 7.46 (s, 1 H), |
| | 7.31-7.39 (m, 2 H), 6.72-6.82 (m, 2 H), 6.66 (dd, J = 8.4, 2.0 Hz, 1 H), 5.06 |
| | (dd, J = 12.8, 5.6 Hz, 1 H), 4.97 (d, J = 5.6 Hz, 4 H), 4.89 (d, J = 6.0 Hz, 2 H), |
| | 4.13 (t, J = 8.4 Hz, 2 H), 3.96 (d, J = 12.4 Hz, 2 H), 3.82 (dd, J = 8.4, 5.6 Hz, 2 H), |
| | 3.52 (s, 2 H), 2.91 (s, 3 H), 2.82-2.88 (m, 2 H), 2.60 (s, 1 H), 2.55 (s, 2 H), |
| | 2.53 (s, 1 H), 1.99-2.05 (m, 1 H), 1.81 (d, J = 11.6 Hz, 2 H), 1.75 (d, J = 2.8 Hz, |
| | 1 H), 1.19-1.29 (m, 2 H) |
| 129 | ¹H NMR (400 MHz, DMSO) δ 11.09 (s, 1H), 8.19 (s, 1H), 7.92-7.86 (m, |
| | 1H), 7.66 (d, J = 8.4 Hz, 1H), 7.37-7.31 (m, 3H), 7.26 (dd, J = 1.6, 8.4 Hz, |
| | 1H), 6.94 (d, J = 5.2 Hz, 2H), 6.75 (d, J = 7.6 Hz, 1H), 5.08 (dd, J = 5.2, 12.8 |
| | Hz, 1H), 5.00-4.92 (m, 4H), 4.89 (d, J = 6.2 Hz, 2H), 4.11 (d, J = 12.4 Hz, |
| | 2H), 4.04 (t, J = 7.6 Hz, 2H), 3.71 (t, J = 6.8 Hz, 2H), 3.51 (s, 2H), 2.95 (d, J = |
| | 12.4 Hz, 2H), 2.90 (s, 3H), 2.90-2.83 (m, 1H), 2.64-2.57 (m, 1H), 2.57- |
| | 2.53 (m, 2H), 2.06-1.99 (m, 1H), 1.78 (d, J = 11.6 Hz, 3H), 1.23-1.11 (m, |
| | 2H) |
| 130 | ¹H NMR (400 MHz, DMSO) δ 11.13 (s, 1H), 8.19 (s, 1H), 7.95-7.79 (m, |
| | 4H), 7.42-7.28 (m, 2H), 6.94 (s, 2H), 6.75 (d, J = 8.0 Hz, 1H), 5.16 (dd, J = |
| | 5.2, 12.8 Hz, 1H), 4.95 (t, J = 5.2 Hz, 4H), 4.89 (d, J = 6.0 Hz, 2H), 3.75-3.65 |
| | (m, 6H), 3.51 (s, 2H), 2.90 (s, 3H), 2.63 (d, J = 2.4 Hz, 2H), 2.57-2.54 (m, |
| | 3H), 2.42-2.38 (m, 2H), 2.12-2.04 (m, 1H), 1.90-1.82 (m, 4H) |
| 131 | ¹H NMR (400 MHz, DMSO) δ 10.95 (s, 1 H), 8.19 (s, 1 H), 8.16 (s, 1 H), 7.88 |
| | (d, J = 8.8 Hz, 1 H), 7.74 (d, J = 8.8 Hz, 1 H), 7.31-7.37 (m, 2 H), 6.94 (d, J = 1.6 |
| | Hz, 2 H), 6.90 (d, J = 9.2 Hz, 1 H), 6.75 (d, J = 7.6 Hz, 1 H), 5.07 (dd, J = 13.2, |
| | 5.2 Hz, 1 H), 4.93-4.98 (m, 4 H), 4.89 (d, J = 6.0 Hz, 2 H), 4.40-4.48 (m, 2 |
| | H), 4.26 (d, J = 17.2 Hz, 1 H), 4.09 (d, J = 17.2 Hz, 1 H), 3.70 (s, 4 H), 3.51 (s, 2 |
| | H), 2.93-2.99 (m, 2 H), 2.90 (s, 3 H), 2.87 (d, J = 5.2 Hz, 1 H), 2.61 (s, 1 H), |
| | 2.55 (s, 1 H), 2.53-2.53 (m, 1 H), 2.38-2.41 (m, 1 H), 2.35 (d, J = 6.4 Hz, 2 |
| | H), 2.14 (d, J = 6.4 Hz, 2 H), 1.93-1.99 (m, 1 H), 1.83-1.90 (m, 1 H), 1.78 (s, |
| | 6 H), 1.02-1.13 (m, 2 H) |
| 132 | ¹H NMR (400 MHz, DMSO) δ 11.08 (s, 1 H), 8.19 (s, 1 H), 7.86-7.92 (m, 1 |
| | H), 7.64 (d, J = 8.4 Hz, 1 H), 7.31-7.39 (m, 2 H), 6.94 (d, J = 1.6 Hz, 2 H), 6.73- |
| | 6.80 (m, 2 H), 6.65 (dd, J = 8.4, 2.0 Hz, 1 H), 5.04-5.09 (m, 1 H), 4.94-4.98 |
| | (m, 4 H), 4.89 (d, J = 6.0 Hz, 2 H), 4.14 (t, J = 8.0 Hz, 2 H), 3.71 (s, 5 H), 3.68 |
| | (s, 1 H), 3.51 (s, 2 H), 2.97-3.06 (m, 2 H), 2.91 (s, 3 H), 2.86 (dd, J = 7.6, 4.4 |
| | Hz, 1 H), 2.61 (s, 3 H), 2.59 (s, 2 H), 2.39 (d, J = 5.2 Hz, 2 H), 1.99-2.05 (m, 1 |
| | H), 1.75-1.81 (m, 4 H) |
| 133 | ¹H NMR (400 MHz, DMSO) δ 11.17-10.97 (m, 1H), 8.19 (s, 1H), 7.92-7.85 |
| | (m, 1H), 7.66 (d, J = 8.4 Hz, 1H), 7.37-7.31 (m, 3H), 7.25 (dd, J = 2.0, 8.8 |
| | Hz, 1H), 6.92 (d, J = 4.4 Hz, 2H), 6.75 (d, J = 7.6 Hz, 1H), 5.07 (dd, J = 5.2, |
| | 12.8 Hz, 1H), 4.99-4.93 (m, 4H), 4.89 (d, J = 6.2 Hz, 2H), 4.13 (t, J = 7.6 Hz, |
| | 2H), 4.07 (d, J = 13.2 Hz, 2H), 3.57 (t, J = 6.4 Hz, 2H), 3.51 (s, 2H), 3.00- |
| | 2.93 (m, 2H), 2.91 (s, 3H), 2.89-2.85 (m, 1H), 2.58 (s, 1H), 2.58-2.53 (m, |
| | 2H), 2.06-2.00 (m, 1H), 1.74 (d, J = 12.0 Hz, 2H), 1.61 (d, J = 5.2 Hz, 3H), |
| | 1.23 (d, J = 12.4 Hz, 2H) |
| 134 | ¹H NMR (400 MHz, DMSO) δ 11.08 (s, 1H), 8.19 (s, 1H), 7.92-7.86 (m, |
| | 1H), 7.64 (d, J = 8.4 Hz, 1H), 7.50 (s, 1H), 7.44 (s, 1H), 7.38-7.32 (m, 2H), |
| | 6.79-6.73 (m, 2H), 6.64 (d, J = 8.4 Hz, 1H), 5.06 (dd, J = 5.2, 12.8 Hz, 1H), |
| | 4.99-4.94 (m, 4H), 4.89 (d, J = 6.0 Hz, 2H), 4.19 (t, J = 8.0 Hz, 2H), 3.91 (d, |
| | J = 12.0 Hz, 2H), 3.67 (t, J = 6.8 Hz, 2H), 3.52 (s, 2H), 2.97-2.92 (m, 1H), |
| | 2.91 (s, 3H), 2.89-2.78 (m, 3H), 2.64-2.53 (m, 2H), 2.05-1.98 (m, 1H), |
| | 1.77 (d, J = 12.0 Hz, 2H), 1.68-1.61 (m, 2H), 1.53 (d, J = 3.2 Hz, 1H), 1.33- |
| | 1.24 (m, 2H) |
| 135 | ¹H NMR (400 MHz, DMSO) δ 0.95-1.28 (m, 3 H) 1.54-1.65 (m, 1 H) 1.71- |
| | 1.83 (m, 6 H) 1.97-2.05 (m, 1 H) 2.35 (br d, J = 7.6 Hz, 2 H) 2.54-2.61 (m, 2 |
| | H) 2.89 (s, 3 H) 2.92-2.97 (m, 2 H) 3.02 (s, 4 H) 3.30 (br d, J = 3.6 Hz, 4 H) |
| | 3.51 (s, 2 H) 3.98-4.08 (m, 2 H) 4.89 (d, J = 6.0 Hz, 2 H) 4.96 (br d, J = 5.6 Hz, |
| | 4 H) 5.07 (dd, J = 12.8, 5.2 Hz, 1 H) 6.75 (d, J = 7.6 Hz, 1 H) 7.23 (br d, J = 8.8 |
| | Hz, 1 H) 7.29-7.37 (m, 3 H) 7.42-7.54 (m, 2 H) 7.65 (d, J = 8.8 Hz, 1 H) |
| | 7.86-7.93 (m, 1 H) 8.19 (s, 1 H) 11.10 (s, 1 H) |
| 136 | ¹H NMR (400 MHz, DMSO) δ 11.13-11.02 (m, 1H), 8.17 (s, 1H), 7.87-7.80 |
| | (m, 1H), 7.65 (d, J = 8.8 Hz, 1H), 7.42 (s, 1H), 7.37-7.27 (m, 2H), 7.27-7.19 |
| | (m, 1H), 6.93 (s, 2H), 6.81 (d, J = 7.2 Hz, 1H), 5.06 (dd, J = 5.2, 12.8 Hz, 1H), |
| | 4.98-4.89 (m, 2H), 4.12-3.98 (m, 2H), 3.69 (s, 4H), 3.30-3.18 (m, 5H), |
| | 2.96 (t, J = 12.4 Hz, 2H), 2.90-2.81 (m, 5H), 2.78 (s, 3H), 2.62-2.53 (m, |
| | 2H), 2.53-2.51 (m, 1H), 2.16-2.08 (m, 2H), 2.04-1.97 (m, 1H), 1.86-1.71 |
| | (m, 7H), 1.27-1.08 (m, 3H) |
| 137 | ¹H NMR (400 MHz, DMSO) δ 11.07 (s, 1H), 8.14 (s, 1H), 7.86 (d, J = 9.2 Hz, |
| | 1H), 7.65 (d, J = 8.8 Hz, 1H), 7.34-7.27 (m, 2H), 7.26-7.19 (m, 2H), 6.93 (s, |
| | 2H), 6.63 (d, J = 7.6 Hz, 1H), 5.06 (dd, J = 5.2, 12.8 Hz, 1H), 4.92 (s, 2H), |
| | 4.04 (d, J = 12.8 Hz, 2H), 3.75 (t, J = 8.8 Hz, 1H), 3.69 (s, 4H), 3.23 (s, 2H), |
| | 3.07-2.85 (m, 5H), 2.71-2.67 (m, 3H), 2.65-2.60 (m, 2H), 2.59-2.53 (m, |
| | 2H), 2.33 (s, 4H), 2.13 (d, J = 5.2 Hz, 2H), 2.05-1.98 (m, 1H), 1.86-1.71 (m, |
| | 7H), 1.21-1.08 (m, 2H) |
| 138 | ¹H NMR (400 MHz, DMSO) δ 10.88 (d, J = 10.8 Hz, 1H), 8.19 (s, 1H), 7.88 |
| | (d, J = 8.8 Hz, 1H), 7.44 (dd, J = 3.2, 8.4 Hz, 1H), 7.39-7.30 (m, 2H), 7.07 (s, |
| | 1H), 7.03-6.97 (m, 1H), 6.94 (d, J = 1.6 Hz, 2H), 6.75 (d, J = 8.0 Hz, 1H), |
| | 5.02-4.92 (m, 4H), 4.89 (d, J = 6.0 Hz, 2H), 4.70-4.49 (m, 2H), 3.94-3.86 |
| | (m, 2H), 3.70 (s, 4H), 3.51 (s, 2H), 2.91 (s, 3H), 2.87-2.80 (m, 2H), 2.80- |
| | 2.76 (m, 1H), 2.65-2.57 (m, 2H), 2.43-2.27 (m, 4H), 2.16 (d, J = 6.0 Hz, |
| | 2H), 2.01-1.93 (m, 1H), 1.78 (s, 7H), 1.41 (dd, J = 6.8, 10.4 Hz, 3H), 1.24- |
| | 1.13 (m, 2H). |
| 139 | ¹H NMR (400 MHz, DMSO) δ 10.98 (s, 1H), 8.19 (s, 1H), 8.16 (s, 1H), 7.92- |
| | 7.85 (m, 1H), 7.50 (s, 1H), 7.48-7.41 (m, 2H), 7.38-7.30 (m, 2H), 7.17 (t, J = |
| | 8.0 Hz, 1H), 6.80-6.69 (m, 1H), 5.07 (dd, J = 5.2, 13.2 Hz, 1H), 4.96 (d, J = |
| | 6.0 Hz, 4H), 4.89 (d, J = 6.0 Hz, 2H), 4.56-4.41 (m, 1H), 4.37-4.24 (m, 1H), |
| | 3.66-3.58 (m, 1H), 3.51 (s, 2H), 3.40-3.35 (m, 4H), 3.30-3.19 (m, 4H), |
| | 2.99-2.92 (m, 2H), 2.90 (s, 3H), 2.89-2.80 (m, 1H), 2.63-2.56 (m, 1H), |
| | 2.44 (s, 4H), 2.14 (d, J = 6.8 Hz, 2H), 2.00-1.88 (m, 5H), 1.81 (d, J = 11.2 |
| | Hz, 2H), 1.62-1.45 (m, 3H), 1.25-1.12 (m, 2H), 0.98-0.83 (m, 2H) |
| 142 | ¹H NMR (400 MHz, DMSO) δ 10.88 (d, J = 12.4 Hz, 1H), 8.19 (s, 1H), 8.15 |
| | (s, 1H), 7.98 (s, 1H), 7.93 (s, 1H), 7.89 (dd, J = 1.2, 8.0 Hz, 1H), 7.70 (dd, J = |
| | 3.2, 8.8 Hz, 1H), 7.40 (s, 1H), 7.36 (t, J = 8.0 Hz, 1H), 6.89 (dd, J = 3.2, 8.8 |
| | Hz, 1H), 6.78 (d, J = 7.6 Hz, 1H), 5.10 (s, 2H), 4.97 (d, J = 6.0 Hz, 2H), 4.89 |
| | (d, J = 6.0 Hz, 2H), 4.70 (dd, J = 5.2, 12.4 Hz, 1H), 4.53-4.38 (m, 1H), 4.16- |
| | 4.01 (m, 2H), 3.75-3.64 (m, 4H), 3.52 (s, 4H), 2.91 (s, 3H), 2.84-2.72 (m, |
| | 1H), 2.63-2.55 (m, 2H), 2.48-2.36 (m, 8H), 2.09 (d, J = 7.2 Hz, 2H), 2.03- |
| | 1.90 (m, 3H), 1.89-1.81 (m, 2H), 1.76 (d, J = 11.6 Hz, 2H), 1.39 (dd, J = 6.8, |
| | 14.4 Hz, 6H), 1.20-1.08 (m, 2H), 0.95-0.80 (m, 2H) |
| 143 | ¹H NMR (400 MHz, DMSO) δ 11.08 (s, 1H), 8.18 (s, 4H), 7.97 (s, 1H), 7.92 |
| | (s, 1H), 7.87 (d, J = 8.8 Hz, 1H), 7.67 (d, J = 8.4 Hz, 1H), 7.41 (s, 1H), 7.37- |
| | 7.31 (m, 2H), 7.24 (d, J = 8.4 Hz, 1H), 6.77 (d, J = 7.6 Hz, 1H), 5.11-5.04 |
| | (m, 3H), 4.96 (d, J = 6.0 Hz, 2H), 4.89 (d, J = 6.0 Hz, 2H), 3.72 (d, J = 13.6 |
| | Hz, 2H), 3.57-3.49 (m, 4H), 3.42 (s, 4H), 2.91 (s, 3H), 2.89-2.75 (m, 5H), |
| | 2.60 (d, J = 2.8 Hz, 1H), 2.57-2.53 (m, 3H), 2.45 (s, 4H), 2.39-2.31 (m, 2H), |
| | 2.16 (dd, J = 6.4, 14.4 Hz, 4H), 2.05-1.93 (m, 6H), 1.79 (d, J = 5.6 Hz, 1H), |
| | 1.70-1.57 (m, 4H), 1.56-1.41 (m, 3H), 1.19-0.96 (m, 5H), 0.88 (d, J = 6.4 |
| | Hz, 3H) |
| 156 | ¹H NMR (400 MHz, DMSO-d6) δ = 11.07-10.83 (m, 1H), 8.19 (s, 1H), 7.97 |
| | (d, J = 19.2 Hz, 2H), 7.91-7.85 (m, 1H), 7.45 (d, J = 8.0 Hz, 1H), 7.40 (s, |
| | 1H), 7.35 (t, J = 8.0 Hz, 1H), 7.16 (t, J = 8.0 Hz, 1H), 6.76 (d, J = 8.0 Hz, 1H), |
| | 5.13-5.03 (m, 3H), 4.97 (d, J = 6.0 Hz, 2H), 4.89 (d, J = 6.0 Hz, 2H), 4.52- |
| | 4.43 (m, 1H), 4.34-4.26 (m, 1H), 3.80-3.66 (m, 2H), 3.64-3.54 (m, 2H), |
| | 3.52 (s, 2H), 3.41-3.34 (m, 2H), 2.96-2.85 (m, 7H), 2.66-2.54 (m, 2H), |
| | 2.42 (dd, J = 4.4, 13.2 Hz, 1H), 2.03-1.86 (m, 6H), 1.74-1.64 (m, 1H), 1.63- |
| | 1.51 (m, 2H), 1.50-1.40 (m, 1H), 1.22-1.12 (m, 1H). |
| 157 | ¹H NMR (400 MHz, DMSO-d6) δ = 10.97 (s, 1H), 8.29 (s, 1H), 8.16 (s, 2H), |
| | 8.07 (s, 1H), 7.96 (s, 1H), 7.90 (s, 1H), 7.69 (dd, J = 1.6, 8.0 Hz, 1H), 7.48- |
| | 7.41 (m, 2H), 7.18-7.10 (m, 2H), 5.20 (s, 2H), 5.07 (dd, J = 4.8, 13.2 Hz, |
| | 1H), 4.47 (d, J = 17.2 Hz, 1H), 4.30 (d, J = 16.8 Hz, 1H), 3.66 (d, J = 16.4 Hz, |
| | 2H), 3.61-3.57 (m, 2H), 3.39-3.32 (m, 4H), 3.22 (s, 3H), 2.97-2.88 (m, |
| | 4H), 2.86 (d, J = 4.0 Hz, 2H), 2.77 (d, J = 8.0 Hz, 2H), 2.60 (s, 4H), 2.41 (dd, |
| | J = 5.2, 13.2 Hz, 2H), 2.31-2.19 (m, 6H), 2.12-2.07 (m, 2H), 1.99 (d, J = 7.2 |
| | Hz, 2H), 1.93-1.86 (m, 4H), 1.81-1.63 (m, 6H), 1.61-1.46 (m, 4H), 1.39- |
| | 1.31 (m, 1H), 1.12-1.07 (m, 4H), 0.96-0.88 (m, 1H), 0.87-0.75 (m, 2H). |
| 158 | ¹H NMR (400 MHz, DMSO-d6) δ: 10.99 (s, 1 H), 8.34 (s, 1 H), 7.85-8.23 |
| | (m, 3 H), 7.74-7.81 (m, 1 H), 7.47 (d, J = 8.0 Hz, 1 H), 7.40 (t, J = 8.0 Hz, 1 H), |
| | 7.17 (t, J = 8.0 Hz, 1 H), 7.08 (s, 1 H), 5.13-5.33 (m, 2 H), 5.08 (dd, J = 12.8, |
| | 5.2 Hz, 1 H), 4.48 (d, J = 17.2 Hz, 1 H), 4.31 (d, J = 16.8 Hz, 1 H), 4.24 (d, |
| | J = 10.8 Hz, 1 H), 3.57-3.65 (m, 1 H), 3.44 (s, 3 H), 3.14-3.26 (m, 4 H), 2.90- |
| | 2.99 (m, 6 H), 2.89 (d, J = 1.2 Hz, 1 H), 2.77-2.83 (m, 1 H), 2.61 (d, J = 3.2 |
| | Hz, 1 H), 2.57 (dd, J = 5.6, 3.2, 2.4 Hz, 1 H), 2.44 (d, J = 2.8 Hz, 1 H), 2.39- |
| | 2.42 (m, 1 H), 2.35-2.39 (m, 1 H), 2.22-2.30 (m, 2 H), 2.05-2.16 (m, 3 H), |
| | 1.88-2.02 (m, 7 H), 1.78-1.85 (m, 5 H), 1.67-1.77 (m, 5 H), 1.53-1.62 (m, |
| | 3 H), 1.10-1.30 (m, 4 H), 0.77-1.09 (m, 5 H), 0.53-0.77 (m, 1 H). |
| 159 | ¹H NMR (400 MHz, DMSO-d6) δ = 10.99 (s, 1H), 8.38 (s, 1H), 7.99 (s, 1H), |
| | 7.92 (d, J = 6.8 Hz, 2H), 7.70-7.63 (m, 1H), 7.49-7.40 (m, 2H), 7.17 (t, J = |
| | 8.0 Hz, 1H), 6.99 (d, J = 7.6 Hz, 1H), 5.20 (s, 2H), 5.08 (dd, J = 5.2, 13.2 Hz, |
| | 1H), 4.48 (d, J = 16.8 Hz, 1H), 4.35-4.28 (m, 1H), 3.81-3.68 (m, 2H), 3.68- |
| | 3.56 (m, 2H), 3.27 (s, 3H), 3.14 (dd, J = 8.0, 10.4 Hz, 2H), 2.98-2.90 (m, |
| | 3H), 2.84-2.78 (m, 1H), 2.77-2.65 (m, 2H), 2.64-2.55 (m, 3H), 2.49-2.31 |
| | (m, 8H), 2.30-2.22 (m, 4H), 2.21-2.06 (m, 4H), 2.03-1.95 (m, 2H), 1.94- |
| | 1.88 (m, 4H), 1.84 (s, 2H), 1.76-1.68 (m, 3H), 1.63-1.53 (m, 3H), 1.51- |
| | 1.39 (m, 2H), 1.14-1.10 (m, 4H), 0.96 (d, J = 8.4 Hz, 1H), 0.92-0.81 (m, |
| | 2H). |
| 160 | ¹H NMR (400 MHz, DMSO-d6) δ: 10.88 (d, J = 12.4 Hz, 1H), 8.19 (s, 1H), |
| | 8.15 (s, 1H), 7.97 (s, 1H), 7.94-7.86 (m, 2H), 7.69 (dd, J = 3.2, 8.8 Hz, 1H), |
| | 7.42-7.31 (m, 2H), 6.88 (dd, J = 3.6, 9.2 Hz, 1H), 6.76 (d, J = 8.0 Hz, 1H), |
| | 5.10 (s, 2H), 4.96 (d, J = 6.0 Hz, 2H), 4.89 (d, J = 6.0 Hz, 2H), 4.69 (dd, J = |
| | 4.8, 12.4 Hz, 1H), 4.51-4.37 (m, 1H), 4.13-4.01 (m, 2H), 3.74-3.61 (m, |
| | 4H), 3.51 (s, 2H), 3.30 (d, J = 10.0 Hz, 4H), 2.90 (s, 3H), 2.84-2.74 (m, 2H), |
| | 2.70-2.64 (m, 1H), 2.61-2.57 (m, 1H), 2.41-2.20 (m, 7H), 2.16-2.09 (m, |
| | 2H), 2.02 (d, J = 6.8 Hz, 4H), 1.94-1.78 (m, 5H), 1.78-1.67 (m, 4H), 1.64- |
| | 1.58 (m, 1H), 1.47 (d, J = 10.4 Hz, 1H), 1.38 (dd, J = 6.8, 14.0 Hz, 6H), 1.18- |
| | 1.04 (m, 2H), 0.97-0.76 (m, 3H). |
| 161 | ¹H NMR (400 MHz, DMSO-d6) δ = 10.99 (s, 1H), 8.51 (s, 1H), 7.98 (d, J = |
| | 6.8 Hz, 2H), 7.92 (s, 1H), 7.81 (d, J = 8.4 Hz, 1H), 7.55-7.43 (m, 2H), 7.16 |
| | (t, J = 8.0 Hz, 1H), 7.09 (d, J = 7.6 Hz, 1H), 5.41 (d, J = 6.0 Hz, 2H), 5.22 (s, |
| | 2H), 5.11 (d, J = 6.0 Hz, 2H), 5.07 (dd, J = 5.2, 13.4 Hz, 1H), 4.48 (d, J = 17.2 |
| | Hz, 1H), 4.31 (d, J = 17.2 Hz, 1H), 3.72-3.58 (m, 3H), 3.47-3.42 (m, 2H), |
| | 3.30-3.26 (m, 4H), 3.21 (s, 1H), 2.98-2.87 (m, 3H), 2.82-2.74 (m, 1H), |
| | 2.72-2.65 (m, 1H), 2.62-2.56 (m, 1H), 2.45-2.34 (m, 1H), 2.33-2.16 (m, |
| | 6H), 2.13-2.03 (m, 3H), 1.97 (d, J = 7.2 Hz, 3H), 1.90 (d, J = 9.6 Hz, 4H), |
| | 1.82-1.65 (m, 5H), 1.61-1.43 (m, 4H), 1.40-1.30 (m, 1H), 1.15-1.07 (m, |
| | 2H), 0.94-0.76 (m, 3H). |
| 162 | ¹H NMR (400 MHz, DMSO-d6) δ: 10.98 (s, 1H), 8.25 (s, 1H), 7.86-8.02 (m, |
| | 2H), 7.71-7.83 (m, 2H), 7.46 (d, J = 8.0 Hz, 1H), 7.32 (t, J = 8.1 Hz, 1H), 7.17 |
| | (t, J = 8.0 Hz, 1H), 7.05 (d, J = 7.6 Hz, 1H), 5.14 (s, 2H), 5.08 (dd, J = 13.2, |
| | 4.8 Hz, 1H), 4.48 (d, J = 17.2 Hz, 1H), 4.31 (d, J = 17.2 Hz, 1H), 3.56-3.75 |
| | (m, 3H), 3.25 (s, 2H), 3.12 (s, 2H), 2.86-2.98 (m, 3H), 2.74-2.83 (m, 1H), 2.57 |
| | (br s, 2H), 2.38 (br s, 1H), 2.16-2.31 (m, 7H), 2.02-2.14 (m, 4H), 1.98 (br d, |
| | J = 7.2 Hz, 3H), 1.91 (br dd, J = 6.8, 2.0 Hz, 4H), 1.62-1.79 (m, 6H), 1.46-1.61 |
| | (m, 4H), 1.22-1.42 (m, 3H), 1.08-1.16 (m, 2H), 0.74-1.01 ppm (m, 8H) |
| 163 | ¹H NMR (400 MHz, DMSO-d6) δ: 10.95 (s, 1H), 8.18 (s, 1H), 8.14 (s, 2H), |
| | 7.97 (s, 1H), 7.93-7.86 (m, 2H), 7.42-7.31 (m, 3H), 7.08 (d, J = 8.4 Hz, 1H), |
| | 6.76 (d, J = 7.6 Hz, 1H), 5.12-5.02 (m, 3H), 4.96 (d, J = 6.0 Hz, 2H), 4.89 (d, |
| | J = 6.0 Hz, 2H), 4.43 (d, J = 16.8 Hz, 1H), 4.25 (d, J = 16.8 Hz, 1H), 3.85 (s, |
| | 3H), 3.73-3.68 (m, 1H), 3.65-3.61 (m, 1H), 3.59-3.55 (m, 1H), 3.51 (s, |
| | 2H), 2.90 (s, 4H), 2.87-2.78 (m, 3H), 2.72-2.63 (m, 2H), 2.60 (s, 2H), 2.47- |
| | 2.39 (m, 2H), 2.34-2.22 (m, 6H), 2.18-2.00 (m, 6H), 1.97-1.87 (m, 5H), |
| | 1.82-1.66 (m, 5H), 1.65-1.51 (m, 4H), 1.51-1.44 (m, 1H), 1.41-1.32 (m, |
| | 1H), 1.17-1.05 (m, 2H), 0.96-0.77 (m, 3H). |
| 164 | ¹H NMR (400 MHz, DMSO-d6) δ = 10.98 (s, 1H), 8.22 (s, 1H), 8.17 (s, 1H), |
| | 7.98 (s, 1H), 7.90 (d, J = 13.2 Hz, 2H), 7.77-7.70 (m, 1H), 7.46 (d, J = 8.0 |
| | Hz, 1H), 7.36 (t, J = 8.0 Hz, 1H), 7.21-7.10 (m, 2H), 5.17 (s, 2H), 5.08 (dd, |
| | J = 4.8, 13.2 Hz, 1H), 4.56-4.40 (m, 1H), 4.39-4.22 (m, 1H), 3.74-3.68 (m, |
| | 1H), 3.67-3.59 (m, 2H), 3.32 (d, J = 4.4 Hz, 4H), 3.15 (s, 3H), 3.02 (s, 2H), |
| | 2.98-2.87 (m, 3H), 2.86-2.68 (m, 2H), 2.64-2.55 (m, 1H), 2.48-2.39 (m, |
| | 4H), 2.30-2.21 (m, 4H), 2.18-2.03 (m, 4H), 2.00 (d, J = 6.8 Hz, 2H), 1.95- |
| | 1.87 (m, 4H), 1.78-1.67 (m, 4H), 1.67-1.62 (m, 1H), 1.61-1.55 (m, 2H), |
| | 1.53-1.49 (m, 1H), 1.47 (s, 6H), 1.41-1.34 (m, 1H), 1.17-1.07 (m, 2H), |
| | 0.94 (s, 1H), 0.89-0.77 (m, 2H) |
| 165 | ¹H NMR (400 MHz, DMSO-d6) δ: 11.00 (s, 1 H), 8.20 (s, 1 H), 7.97 (s, 1 H), |
| | 7.88-7.94 (m, 2 H), 7.64 (d, J = 8.0 Hz, 1 H), 7.40 (s, 1 H), 7.36 (t, J = 8.0 Hz, 1 |
| | H), 7.27 (d, J = 8.0 Hz, 1 H), 6.76 (d, J = 7.2 Hz, 1 H), 5.11 (s, 3 H), 4.97 (d, |
| | J = 6.0 Hz, 2 H), 4.89 (d, J = 6.0 Hz, 2 H), 4.35-4.52 (m, 1 H), 4.17-4.34 (m, 1 |
| | H), 3.58-3.75 (m, 3 H), 3.52 (s, 2 H), 3.28 (dd, J = 5.2, 2.4 Hz, 2 H), 2.91 (s, 3 |
| | H), 2.87 (d, J = 5.6 Hz, 2 H), 2.75-2.83 (m, 1 H), 2.60 (d, J = 6.4 Hz, 2 H), 2.56 |
| | (s, 1 H), 2.36-2.43 (m, 2 H), 2.15-2.29 (m, 6 H), 2.05-2.14 (m, 3 H), 1.99 |
| | (d, J = 6.8 Hz, 3 H), 1.85-1.97 (m, 5 H), 1.67-1.83 (m, 5 H), 1.53-1.66 (m, 4 |
| | H), 1.48 (d, J = 11.2 Hz, 1 H), 1.32-1.40 (m, 1 H), 1.08-1.17 (m, 2 H), 0.78- |
| | 0.94 (m, 3 H). |
| 166 | ¹H NMR (400 MHz, DMSO-d6) δ = 10.98 (s, 1H), 8.19 (s, 1H), 8.16 (s, 1H), |
| | 8.05-7.84 (m, 3H), 7.46 (d, J = 8.0 Hz, 1H), 7.40 (s, 1H), 7.36 (t, J = 8.0 Hz, |
| | 1H), 7.17 (t, J = 8.0 Hz, 1H), 6.77 (d, J = 8.0 Hz, 1H), 5.24-5.03 (m, 3H), |
| | 4.97 (d, J = 6.0 Hz, 2H), 4.90 (d, J = 6.0 Hz, 2H), 4.48 (d, J = 17.2 Hz, 1H), |
| | 4.31 (d, J = 16.8 Hz, 1H), 3.76-3.68 (m, 1H), 3.67-3.59 (m, 2H), 3.52 (s, |
| | 2H), 3.48-3.39 (m, 2H), 2.95 (d, J = 8.0 Hz, 2H), 2.91 (s, 3H), 2.88 (s, 1H), |
| | 2.73 (s, 1H), 2.61 (d, J = 1.2 Hz, 2H), 2.56 (d, J = 8.0 Hz, 2H), 2.54-2.52 (m, |
| | 4H), 2.48-2.36 (m, 3H), 2.31-2.22 (m, 1H), 2.13-2.04 (m, 2H), 2.03-1.96 |
| | (m, 3H), 1.91 (d, J = 11.2 Hz, 5H), 1.72 (d, J = 2.4 Hz, 4H), 1.67-1.50 (m, |
| | 4H), 1.49-1.32 (m, 2H), 1.18-1.08 (m, 2H), 0.94 (d, J = 6.0 Hz, 2H), 0.90 |
| | (d, J = 6.0 Hz, 1H), 0.84 (d, J = 11.6 Hz, 2H) |
| 167 | ¹H NMR (400 MHz, DMSO-d6) δ: 10.84-11.18 (m, 1H), 8.15 (s, 1H), 7.93- |
| | 8.01 (m, 2H), 7.88 (dd, J = 8.0, 1.6 Hz, 1H), 7.47 (d, J = 8.0 Hz, 1H), 7.33 (t, |
| | J = 8.0 Hz, 1H), 7.26 (s, 1H), 7.17 (t, J = 8.4 Hz, 1H), 6.67 (br d, J = 7.6 Hz, |
| | 1H), 5.02-5.12 (m, 3H), 4.48 (d, J = 16.8 Hz, 1H), 4.31 (d, J = 17.2 Hz, 1H), |
| | 3.70-3.86 (m, 3H), 3.56-3.67 (m, 1H), 3.24 (s, 2H), 3.06 (br t, J = 10.4 Hz, |
| | 2H), 2.87-2.98 (m, 3H), 2.70 (s, 3H), 2.57-2.66 (m, 7H), 2.37-2.42 (m, 4H), |
| | 1.88-2.03 (m, 8H), 1.72-1.81 (m, 3H), 1.49-1.63 (m, 4H), 1.09-1.22 (m, 3H), |
| | 0.81-0.99 ppm (m, 3H) |
| 168 | ¹H NMR (400 MHz, DMSO-d6) δ: 10.99 (s, 1H), 8.15 (d, J = 4.8 Hz, 2H), |
| | 7.92-8.06 (m, 2H), 7.88 (br d, J = 7.6 Hz, 1H), 7.47 (d, J = 8.0 Hz, 1H), 7.34 |
| | (t, J = 8.0 Hz, 1H), 7.26 (s, 1H), 7.17 (br t, J = 8.0 Hz, 1H), 6.68 (br d, J = 6.8 |
| | Hz, 1H), 6.46-6.61 (m, 1H), 5.04-5.14 (m, 3H), 4.44-4.52 (m, 1H), 4.31 (br d, |
| | J = 17.2 Hz, 1H), 3.69-3.86 (m, 3H), 3.56-3.67 (m, 2H), 3.01-3.11 (m, 3H), |
| | 2.82-2.99 (m, 6H), 2.71 (s, 5H), 2.57-2.66 (m, 6H), 2.38-2.45 (m, 4H), 1.86- |
| | 2.02 (m, 9H), 1.66-1.80 (m, 5H), 1.42-1.62 (m, 6H), 1.08-1.23 (m, 3H), 0.84- |
| | 1.06 ppm (m, 4H). |
| 169 | ¹H NMR (400 MHz, DMSO-d6) δ: 10.95 (s, 1H), 8.18 (s, 1H), 7.87 (dd, J = |
| | 1.6, 8.0 Hz, 1H), 7.41-7.28 (m, 3H), 7.08 (d, J = 8.4 Hz, 1H), 6.93 (d, J = 2.0 |
| | Hz, 2H), 6.74 (d, J = 7.6 Hz, 1H), 5.06 (dd, J = 5.2, 13.2 Hz, 1H), 4.99-4.91 |
| | (m, 4H), 4.88 (d, J = 6.0 Hz, 2H), 4.43 (d, J = 17.2 Hz, 1H), 4.26 (d, J = 17.2 |
| | Hz, 1H), 3.86 (s, 3H), 3.68 (s, 4H), 3.62-3.56 (m, 1H), 3.50 (s, 2H), 3.39- |
| | 3.36 (m, 2H), 2.94-2.79 (m, 6H), 2.60 (d, J = 2.4 Hz, 1H), 2.57-2.54 (m, |
| | 1H), 2.52 (s, 2H), 2.45-2.40 (m, 2H), 2.39-2.32 (m, 1H), 2.14-2.09 (m, |
| | 2H), 1.99-1.90 (m, 5H), 1.79-1.72 (m, 6H), 1.62-1.58 (m, 2H), 1.52-1.41 |
| | (m, 1H), 1.24-1.09 (m, 2H), 0.98-0.81 (m, 2H). |
| 170 | ¹H NMR (400 MHz, DMSO-d6) δ = 10.97 (s, 1H), 8.17 (s, 1H), 7.87-7.81 |
| | (m, 1H), 7.44 (s, 1H), 7.34 (t, J = 8.0 Hz, 1H), 7.22 (s, 2H), 6.87 (d, J = 7.6 |
| | Hz, 1H), 6.82 (d, J = 7.6 Hz, 1H), 5.03 (dd, J = 4.8, 13.2 Hz, 1H), 4.94 (s, |
| | 2H), 4.42-4.32 (m, 1H), 4.22 (d, J = 16.8 Hz, 1H), 3.70-3.57 (m, 4H), 3.46 |
| | (d, J = 9.6 Hz, 2H), 3.30-3.22 (m, 4H), 2.96-2.87 (m, 3H), 2.86 (d, J = 2.8 |
| | Hz, 2H), 2.78 (s, 5H), 2.72 (s, 1H), 2.71-2.66 (m, 1H), 2.63-2.54 (m, 4H), |
| | 2.45-2.40 (m, 1H), 2.39-2.31 (m, 3H), 2.00-1.94 (m, 1H), 1.89 (s, 2H), |
| | 1.74 (d, J = 12.0 Hz, 2H), 1.65-1.56 (m, 1H), 1.28-1.15 (m, 2H). |
| 17 | ¹H NMR (400 MHz, DMSO-d6) δ = 10.97 (s, 1H), 8.18 (s, 1H), 7.85 (dd, J = |
| | 1.6, 8.0 Hz, 1H), 7.43 (s, 1H), 7.34 (t, J = 8.0 Hz, 1H), 6.95 (s, 2H), 6.90 (d, |
| | J = 7.6 Hz, 1H), 6.82 (d, J = 7.6 Hz, 1H), 5.03 (dd, J = 5.2, 13.2 Hz, 1H), 4.96 |
| | (s, 2H), 4.40 (d, J = 16.8 Hz, 1H), 4.28-4.20 (m, 1H), 3.70 (s, 4H), 3.54- |
| | 3.47 (m, 2H), 3.28-3.19 (m, 2H), 2.89 (s, 3H), 2.87 (s, 2H), 2.79 (s, 3H), |
| | 2.79-2.72 (m, 2H), 2.64-2.54 (m, 6H), 2.47-2.34 (m, 4H), 2.18 (d, J = 7.2 Hz, |
| | 2H), 1.99-1.94 (m, 1H), 1.89-1.75 (m, 6H), 1.74-1.67 (m, 1H), 1.35-1.20 |
| | (m, 2H). |
| 172 | ¹H NMR (400 MHz, DMSO-d6) δ: 11.08 (s, 1 H), 8.17 (s, 1 H), 7.81-7.87 |
| | (m, 1 H), 7.68 (d, J = 8.4 Hz, 1 H), 7.42 (s, 1 H), 7.38 (s, 1 H), 7.26-7.36 (m, 2 |
| | H), 6.93 (d, J = 2.0 Hz, 2 H), 6.81 (d, J = 7. 6 Hz, 1 H), 5.07 (dd, J = 12.8, 5.6 Hz, |
| | 1 H), 4.95 (s, 2 H), 3.88 (d, J = 13.2 Hz, 2 H), 3.69 (s, 4 H), 3.36 (s, 1 H), 3.30- |
| | 3.30 (m, 1 H), 3.30 (s, 2 H), 3.21-3.28 (m, 2 H), 2.88 (s, 2 H), 2.86 (s, 2 H), |
| | 2.78 (s, 3 H), 2.58-2.64 (m, 1 H), 2.55 (d, J = 4.8 Hz, 2 H), 2.52 (d, J = 2.0 Hz, |
| | 2 H), 2.45-2.48 (m, 2 H), 2.44 (s, 1 H), 1.99-2.05 (m, 1 H), 1.88-1.97 (m, 2 |
| | H), 1.76 (s, 6 H). |
| 173 | ¹H NMR (400 MHz, DMSO-d6) δ = 10.99 (s, 1H), 8.18 (s, 1H), 7.85 (d, J = |
| | 8.4 Hz, 1H), 7.70-7.64 (m, 1H), 7.43 (s, 1H), 7.37-7.32 (m, 1H), 7.32-7.20 |
| | (m, 1H), 6.95 (s, 2H), 6.82 (d, J = 7.6 Hz, 1H), 5.16-5.06 (m, 1H), 4.96 (s, |
| | 2H), 4.40 (s, 1H), 4.28 (s, 1H), 3.71 (s, 4H), 3.25 (d, J = 7.6 Hz, 3H), 2.91 (s, |
| | 1H), 2.88 (d, J = 8.0 Hz, 4H), 2.79 (s, 3H), 2.78-2.69 (m, 2H), 2.65-2.55 (m, |
| | 2H), 2.47 (s, 2H), 2.36 (d, J = 3.2 Hz, 2H), 2.33-2.27 (m, 1H), 2.20 (d, J = |
| | 6.8 Hz, 2H), 2.12-1.89 (m, 2H), 1.85 (d, J = 13.2 Hz, 2H), 1.79 (s, 4H), |
| | 1.74-1.67 (m, 1H), 1.41-1.26 (m, 2H) |
| 174 | ¹H NMR (400 MHz, DMSO-d6) δ: 10.97 (s, 1H), 8.16 (d, J = 4.4 Hz, 2H), |
| | 7.84 (d, J = 9.2 Hz, 1H), 7.44-7.29 (m, 3H), 7.08 (d, J = 8.0 Hz, 1H), 6.94 (s, |
| | 2H), 6.81 (d, J = 8.0 Hz, 1H), 5.06 (dd, J = 5.2, 13.2 Hz, 1H), 4.95 (s, 2H), |
| | 4.43 (d, J = 17.2 Hz, 1H), 4.26 (d, J = 16.8 Hz, 1H), 3.86 (s, 3H), 3.69 (s, 4H), |
| | 3.57-3.53 (m, 2H), 3.30-3.25 (m, 4H), 2.92-2.84 (m, 5H), 2.78 (s, 3H), |
| | 2.69-2.59 (m, 3H), 2.40-2.30 (m, 4H), 2.19 (d, J = 6.8 Hz, 2H), 2.00-1.93 |
| | (m, 1H), 1.86-1.75 (m, 6H), 1.72-1.65 (m, 1H), 1.36-1.24 (m, 2H). |
| 175 | ¹H NMR (400 MHz, DMSO-d6) δ: 10.88 (d, J = 12.4 Hz, 1H), 8.16 (d, J = 4.8 |
| | Hz, 2H), 7.84 (dd, J = 1.2, 8.0 Hz, 1H), 7.69 (dd, J = 3.2, 8.8 Hz, 1H), 7.42 (s, |
| | 1H), 7.33 (t, J = 8.0 Hz, 1H), 6.93 (s, 2H), 6.88 (dd, J = 3.6, 8.8 Hz, 1H), 6.81 |
| | (d, J = 8.0 Hz, 1H), 4.94 (s, 2H), 4.71-4.66 (m, 1H), 4.52-4.38 (m, 1H), 4.13- |
| | 4.02 (m, 2H), 3.73-3.65 (m, 5H), 3.24 (d, J = 8.0 Hz, 2H), 2.87 (d, J = 8.0 |
| | Hz, 4H), 2.78 (s, 4H), 2.61-2.57 (m, 2H), 2.52 (s, 2H), 2.31 (d, J = 4.0 Hz, |
| | 4H), 2.06 (d, J = 7.2 Hz, 2H), 1.98-1.90 (m, 3H), 1.90-1.81 (m, 3H), 1.79- |
| | 1.71 (m, 6H), 1.47-1.33 (m, 7H), 1.21-1.09 (m, 2H), 0.94-0.82 (m, 2H). |
| 176 | ¹H NMR (400 MHz, DMSO-d6) δ: 10.96 (s, 1H), 8.16 (d, J = 1.2 Hz, 2H), |
| | 7.84 (dd, J = 1.2, 8.0 Hz, 1H), 7.42 (s, 1H), 7.33 (t, J = 8.0 Hz, 1H), 6.95 (d, |
| | J = 4.8 Hz, 2H), 6.89 (d, J = 7.2 Hz, 1H), 6.81 (d, J = 8.0 Hz, 1H), 5.02 (dd, J = |
| | 5.2, 13.2 Hz, 1H), 4.95 (s, 2H), 4.39 (d, J = 16.8 Hz, 1H), 4.23 (d, J = 16.8 Hz, |
| | 1H), 3.97-3.84 (m, 4H), 3.49 (d, J = 8.8 Hz, 3H), 3.24 (d, J = 8.0 Hz, 4H), |
| | 2.87 (d, J = 8.0 Hz, 4H), 2.80-2.72 (m, 7H), 2.58-2.55 (m, 2H), 2.53 (s, 3H), |
| | 2.43-2.37 (m, 1H), 2.35-2.31 (m, 2H), 2.09-2.05 (m, 2H), 2.00-1.91 (m, |
| | 1H), 1.89-1.80 (m, 2H), 1.69-1.56 (m, 1H), 1.36-1.21 (m, 2H). |
| 177 | ¹H NMR (400 MHz, DMSO-d6) δ: 10.96 (s, 1H), 8.17 (d, J = 4.4 Hz, 2H), |
| | 7.86 (br d, J = 8.0 Hz, 1H), 7.44 (s, 1H), 7.34 (t, J = 8.0 Hz, 1H), 7.01 (s, 2H), |
| | 6.89 (br d, J = 7.6 Hz, 1H), 6.81 (d, J = 7.6 Hz, 1H), 5.03 (dd, J = 13.2, 4.8 |
| | Hz, 1H), 4.95 (s, 2H), 4.21-4.42 (m, 2H), 3.41-3.45 (m, 4H), 3.36 (br s, 5H), |
| | 3.30 (br s, 3H), 3.24 (br s, 2H), 2.88 (br d, J = 8.0 Hz, 5H), 2.80 (s, 3H), 2.68- |
| | 2.76 (m, 2H), 2.56-2.63 (m, 3H), 2.41 (br dd, J = 13.6, 4.1 Hz, 1H), 2.35 (br d, |
| | J = 7.2 Hz, 2H), 2.02-2.08 (m, 1H), 1.93-2.01 (m, 2H), 1.77-1.87 (m, 4H), |
| | 1.58-1.68 (m, 1H), 1.24-1.35 ppm (m, 2H) |
| 178 | ¹H NMR (400 MHz, DMSO-d6) δ: 10.87 (s, 1H), 8.17 (d, J = 5.2 Hz, 2H), |
| | 7.84 (dd, J = 1.2, 8.0 Hz, 1H), 7.69 (dd, J = 3.2, 8.8 Hz, 1H), 7.42 (s, 1H), |
| | 7.33 (t, J = 8.0 Hz, 1H), 6.95 (d, J = 4.4 Hz, 2H), 6.88 (dd, J = 3.2, 8.8 Hz, |
| | 1H), 6.81 (d, J = 7.6 Hz, 1H), 4.95 (s, 2H), 4.72-4.66 (m, 1H), 4.52-4.37 (m, |
| | 1H), 4.14-4.02 (m, 2H), 3.93-3.84 (m, 4H), 3.81-3.71 (m, 1H), 3.27-3.19 |
| | (m, 6H), 2.90-2.85 (m, 4H), 2.78 (s, 3H), 2.70 (s, 2H), 2.62-2.57 (m, 2H), |
| | 2.54-2.50 (m, 2H), 2.27-2.18 (m, 2H), 2.08-2.00 (m, 2H), 1.99-1.90 (m, |
| | 3H), 1.88-1.78 (m, 4H), 1.45-1.36 (m, 7H), 1.21-1.08 (m, 2H), 0.96-0.83 |
| | (m, 2H). |
| 179 | ¹H NMR (400 MHz, DMSO-d6) δ: 10.88 (d, J = 12.4 Hz, 1H), 8.18 (d, J = |
| | 1.2 Hz, 2H), 7.78-7.94 (m, 1H), 7.70 (dd, J = 8.8, 3.2 Hz, 1H), 7.44 (s, 1H), |
| | 7.34 (t, J = 8.0 Hz, 1H), 7.01 (s, 2H), 6.89 (dd, J = 8.8, 3.2 Hz, 1H), 6.81 (d, |
| | J = 7.2 Hz, 1H), 4.95 (s, 2H), 4.66-4.75 (m, 1H), 4.39-4.51 (m, 1H), 4.04-4.14 |
| | (m, 2H), 3.71 (br s, 1H), 3.41 (br s, 4H), 3.34-3.36 (m, 4H), 3.30 (br s, 3H), |
| | 3.22 (br s, 1H), 2.88 (br d, J = 8.0 Hz, 4H), 2.80 (s, 3H), 2.58-2.64 (m, 2H), |
| | 2.42-2.47 (m, 2H), 2.23 (br d, J = 7.2 Hz, 2H), 2.02 (br d, J = 5.6 Hz, 1H), |
| | 1.91-1.99 (m, 4H), 1.77-1.88 (m, 6H), 1.39 (br dd, J = 14.2, 6.8 Hz, 7H), 1.09- |
| | 1.20 (m, 2H), 0.84-0.97 ppm (m, 2H) |
| 180 | ¹H NMR (400 MHz, DMSO-d6) δ = 10.65 (d, J = 10.4 Hz, 1H), 8.13-8.09 |
| | (m, 1H), 7.80 (dd, J = 1.6, 8.0 Hz, 1H), 7.69 (d, J = 8.8 Hz, 1H), 7.51-7.35 |
| | (m, 1H), 7.34-7.30 (m, 1H), 7.21 (d, J = 12.8 Hz, 2H), 6.90-6.86 (m, 1H), |
| | 6.85-6.69 (m, 1H), 4.96-4.85 (m, 2H), 4.76-4.62 (m, 1H), 4.54-4.39 (m, |
| | 1H), 4.10-3.98 (m, 2H), 3.71 (dt, J = 4.4, 8.4 Hz, 1H), 3.67-3.57 (m, 4H), |
| | 3.48-3.33 (m, 3H), 3.31 (s, 2H), 3.26-3.19 (m, 2H), 2.90 (d, J = 8.0 Hz, 3H), |
| | 2.88 (s, 2H), 2.81-2.76 (m, 3H), 2.64 (dd, J = 4.0, 10.0 Hz, 2H), 2.58 (d, J = |
| | 4.0 Hz, 2H), 2.26 (d, J = 6.4 Hz, 2H), 2.06-1.99 (m, 1H), 1.94-1.82 (m, 6H), |
| | 1.76-1.69 (m, 2H), 1.48-1.35 (m, 6H), 1.21-1.06 (m, 3H), 0.93-0.82 (m, |
| | 2H). |
| 181 | ¹H NMR (400 MHz, DMSO-d6) δ = 10.92-10.84 (m, 1H), 8.17 (s, 1H), 7.88- |
| | 7.80 (m, 1H), 7.73-7.63 (m, 1H), 7.41 (s, 1H), 7.36-7.29 (m, 1H), 7.09 (s, |
| | 2H), 6.87 (dd, J = 3.2, 8.8 Hz, 1H), 6.81 (d, J = 7.2 Hz, 1H), 4.93 (s, 2H), |
| | 4.74-4.63 (m, 1H), 4.59-4.51 (m, 1H), 4.50-4.37 (m, 1H), 4.15-3.98 (m, 2H), |
| | 3.73-3.64 (m, 1H), 3.55 (s, 1H), 3.45 (d, J = 8.0 Hz, 1H), 3.22 (s, 7H), 2.87 |
| | (d, J = 8.0 Hz, 5H), 2.79-2.76 (m, 3H), 2.62-2.55 (m, 2H), 2.28 (d, J = 6.0 |
| | Hz, 2H), 2.01-1.70 (m, 10H), 1.41-1.34 (m, 5H), 1.29-1.03 (m, 4H), 0.91- |
| | 0.80 (m, 2H). |
| 182 | ¹H NMR (400 MHz, DMSO-d6) δ = 10.88 (d, J = 12.4 Hz, 1H), 8.18 (s, 1H), |
| | 7.87-7.84 (m, 1H), 7.69 (d, J = 3.2, 8.8 Hz, 1H), 7.42 (s, 1H), 7.36-7.32 (m, |
| | 1H), 7.10 (s, 1H), 6.88 (d, J = 3.6, 8.8 Hz, 1H), 6.82 (d, J = 7.6 Hz, 1H), 4.94 |
| | (s, 2H), 4.72-4.68 (m, 1H), 4.54 (s, 1H), 4.48 (q, J = 6.4 Hz, 1H), 4.41 (q, J = |
| | 6.8 Hz, 1H), 3.54 (s, 2H), 3.46 (s, 1H), 3.44 (s, 1H), 3.27 (s, 2H), 3.25 (s, 2H), |
| | 2.92 (s, 1H), 2.88 (d, J = 8.4 Hz, 5H), 2.68-2.67 (m, 1H), 2.62-2.57 (m, 3H), |
| | 2.46-2.37 (m, 4H), 2.36-2.31 (m, 2H), 2.27 (d, J = 6.4 Hz, 2H), 1.94-1.79 |
| | (m, 10H), 1.38 (d, J = 7.6 Hz, 5H), 1.11-1.04 (m, 2H), 0.86 (d, J = 12.4 Hz, |
| | 2H). |
| 183 | ¹H NMR (400 MHz, DMSO-d6) δ = 11.18-10.62 (m, 1H), 8.29-8.08 (m, |
| | 1H), 8.02-7.76 (m, 1H), 7.75-7.60 (m, 1H), 7.50-7.39 (m, 1H), 7.38-7.18 |
| | (m, 3H), 6.96-6.76 (m, 2H), 5.07-4.85 (m, 2H), 4.79-4.63 (m, 1H), 4.58- |
| | 4.41 (m, 3H), 4.36 (d, J = 4.0 Hz, 1H), 4.17-4.03 (m, 2H), 3.84-3.62 (m, |
| | 2H), 3.52-3.43 (m, 4H), 2.89 (s, 2H), 2.78 (s, 2H), 2.27 (d, J = 1.2 Hz, 4H), |
| | 1.92 (s, 12H), 1.77-1.67 (m, 4H), 1.45-1.34 (m, 6H), 1.20-1.03 (m, 4H), |
| | 0.96-0.71 (m, 3H) |
| 184 | ¹H NMR (400 MHz, DMSO-d6) δ = 10.99 (s, 1H), 8.22-8.14 (m, 1H), 7.88 |
| | (d, J = 8.8 Hz, 1H), 7.70-7.60 (m, 1H), 7.37 (s, 1H), 7.36-7.31 (m, 1H), 7.28 |
| | (d, J = 8.0 Hz, 1H), 6.94 (d, J = 6.8 Hz, 2H), 6.76 (d, J = 7.6 Hz, 1H), 5.10 |
| | (dd, J = 5.2, 12.8 Hz, 1H), 5.04-4.91 (m, 4H), 4.89 (d, J = 6.0 Hz, 2H), 4.62- |
| | 4.13 (m, 2H), 3.73 (s, 4H), 3.65 (t, J = 8.4 Hz, 1H), 3.51 (s, 2H), 3.31-3.29 |
| | (m, 4H), 3.29-3.25 (m, 2H), 2.98-2.84 (m, 6H), 2.60 (d, J = 16.0 Hz, 2H), |
| | 2.56-2.53 (m, 2H), 2.46 (d, J = 8.4 Hz, 2H), 1.96 (dd, J = 4.0, 6.8 Hz, 6H), |
| | 1.86 (s, 2H), 1.83-1.77 (m, 2H), 1.62 (d, J = 9.6 Hz, 3H), 1.26-1.13 (m, 2H), |
| | 1.01-0.85 (m, 2H) |
| 185 | ¹H NMR (400 MHz, DMSO-d6) δ: 10.97 (s, 1H), 8.19 (s, 1H), 7.93-7.82 (m, |
| | 1H), 7.43-7.28 (m, 3H), 7.18-7.09 (m, 1H), 6.98-6.88 (m, 2H), 6.79-6.68 |
| | (m, 1H), 5.10-5.03 (m, 1H), 4.99-4.91 (m, 4H), 4.88 (d, J = 6.1 Hz, 2H), |
| | 4.51-4.40 (m, 1H), 4.34-4.23 (m, 1H), 3.88 (s, 3H), 3.74-3.66 (m, 4H), |
| | 3.50 (s, 2H), 3.02-2.91 (m, 3H), 2.89 (s, 3H), 2.62-2.55 (m, 5H), 2.45-2.43 |
| | (m, 5H), 2.03-1.91 (m, 4H), 1.78 (s, 5H) |
| 186 | ¹H NMR (400 MHz, DMSO-d6) δ: 10.96 (s, 1H), 8.20-8.14 (m, 1H), 7.84 (d, |
| | J = 9.2 Hz, 1H), 7.44-7.29 (m, 3H), 7.08 (d, J = 8.0 Hz, 1H), 6.93 (s, 2H), |
| | 6.81 (d, J = 8.0 Hz, 1H), 5.06 (dd, J = 5.2, 13.6 Hz, 1H), 4.94 (s, 2H), 4.43 (d, |
| | J = 16.8 Hz, 1H), 4.31-4.20 (m, 1H), 3.86 (s, 3H), 3.68 (s, 4H), 3.59 (dd, J = |
| | 2.8, 9.2 Hz, 1H), 3.24-3.15 (m, 5H), 2.91-2.81 (m, 7H), 2.78 (s, 3H), 2.59 |
| | (d, J = 3.6 Hz, 2H), 2.32-2.23 (m, 4H), 2.08-2.01 (m, 2H), 2.01-1.87 (m, |
| | 6H), 1.80-1.71 (m, 6H), 1.64-1.54 (m, 2H), 1.50-1.39 (m, 1H), 1.20-1.09 |
| | (m, 2H), 0.94-0.81 (m, 2H). |
| 187 | ¹H NMR (400 MHz, DMSO-d6) δ = 10.97 (s, 1H), 8.18 (s, 1H), 8.15 (s, 1H), |
| | 7.84 (d, J = 8.4 Hz, 1H), 7.43 (s, 1H), 7.34 (t, J = 8.0 Hz, 1H), 6.94 (d, J = 1.6 |
| | Hz, 2H), 6.90 (d, J = 7.6 Hz, 1H), 6.82 (d, J = 7.6 Hz, 1H), 5.03 (dd, J = 4.8, |
| | 13.2 Hz, 1H), 4.95 (s, 2H), 4.44-4.35 (m, 1H), 4.25-4.18 (m, 1H), 3.69 (s, |
| | 4H), 3.66-3.52 (m, 2H), 3.25-3.19 (m, 2H), 2.97-2.90 (m, 3H), 2.89 (s, |
| | 2H), 2.87 (s, 2H), 2.79 (s, 3H), 2.61 (d, J = 2.4 Hz, 1H), 2.55 (s, 2H), 2.54 (s, |
| | 3H), 2.44-2.30 (m, 5H), 2.12 (d, J = 6.0 Hz, 2H), 2.01-1.86 (m, 6H), 1.78 (s, |
| | 6H), 1.60-1.53 (m, 2H), 1.50-1.43 (m, 1H), 1.21-1.12 (m, 2H), 0.94-0.84 |
| | (m, 2H). |
| 188 | ¹H NMR (400 MHz, DMSO-d6) δ = 10.95-11.00 (m, 1 H), 8.18 (s, 1 H), 7.82- |
| | 7.90 (m, 1 H), 7.38-7.48 (m, 1 H), 7.29-7.37 (m, 1 H), 7.02 (d, J = 15.2 Hz, |
| | 2 H), 6.90 (d, J = 7.6 Hz, 1 H), 6.84 (br d, J = 7.6 Hz, 1 H), 5.00-5.06 (m, 1 H), |
| | 4.96 (s, 2 H), 4.36-4.45 (m, 1 H), 4.19-4.29 (m, 1 H), 3.53-3.81 (m, 4 H), |
| | 3.44-3.49 (m, 3 H), 3.23 (d, J = 8.8 Hz, 2 H), 3.06 ( d, J = 1.2 Hz, 2 H), 2.83- |
| | 2.96 (m, 8 H), 2.81 (s, 3 H), 2.57 (s, 1 H), 2.53 (s, 3 H), 2.36-2.43 (m, 1 H), |
| | 2.03-2.29 (m, 3 H), 1.86-2.03 (m, 7 H), 1.75-1.86 (m, 2 H), 1.63-1.72 (m, |
| | 1 H), 1.56 ( d, J = 8.8 Hz, 2 H), 1.12-1.28 (m, 2 H), 1.04 (d, J = 11.2 Hz, 2 H) |
| 189 | ¹H NMR (400 MHz, DMSO-d6) δ = 10.97 (s, 1H), 8.17 (s, 1H), 7.88-7.82 |
| | (m, 1H), 7.42 (s, 1H), 7.34 (t, J = 8.0 Hz, 1H), 6.95 (d, J = 7.6 Hz, 2H), 6.89 |
| | (d, J = 7.2 Hz, 1H), 6.82 (d, J = 8.0 Hz, 1H), 5.03 (dd, J = 5.2, 12.8 Hz, 1H), |
| | 4.96 (s, 2H), 4.44-4.37 (m, 1H), 4.21-4.25 (m, 1H), 4.04 (s, 4H), 3.47 (dd, |
| | J = 4.4, 6.8 Hz, 2H), 3.30-3.19 (m, 4H), 2.88 (d, J = 8.0 Hz, 5H), 2.79 (s, 3H), |
| | 2.75 (d, J = 12.0 Hz, 2H), 2.64-2.54 (m, 3H), 2.54 (s, 3H), 2.41 (dd, J = 4.8, |
| | 12.8 Hz, 1H), 2.31 (d, J = 6.4 Hz, 2H), 1.99-1.93 (m, 1H), 1.80-1.75 (m, |
| | 2H), 1.47-1.40 (m, 1H), 1.33-1.22 (m, 3H). |
| 190 | ¹H NMR (400 MHz, DMSO-d6) δ = 10.98 (s, 1H), 8.23-8.15 (m, 1H), 8.14 |
| | (s, 1H), 7.76 (dd, J = 1.6, 8.0 Hz, 1H), 7.33-7.24 (m, 2H), 6.94 (s, 2H), 6.90 |
| | (d, J = 7.6 Hz, 1H), 6.66 (d, J = 6.8 Hz, 1H), 5.08-4.98 (m, 1H), 4.92 (s, 2H), |
| | 4.53-4.33 (m, 1H), 4.24 (d, J = 16.8 Hz, 1H), 3.70 (s, 3H), 3.54-3.46 (m, |
| | 2H), 3.19 (s, 2H), 2.94-2.87 (m, 1H), 2.79-2.72 (m, 2H), 2.71-2.68 (m, |
| | 3H), 2.59 (d, J = 3.2 Hz, 1H), 2.54 (s, 5H), 2.44-2.42 (m, 1H), 2.40-2.37 (m, |
| | 2H), 2.36-2.31 (m, 4H), 2.21-2.14 (m, 3H), 1.99-1.93 (m, 1H), 1.92-1.74 |
| | (m, 8H), 1.72-1.64 (m, 1H), 1.32-1.23 (m, 2H), 1.17-0.91 (m, 2H) |
| 191 | ¹H NMR (400 MHz, DMSO-d₆) δ = 10.99 (s, 1H), 8.25 (s, 1H), 7.77 (d, J = |
| | 8.0 Hz, 1H), 7.72 (s, 1H), 7.31 (t, J = 8.0 Hz, 1H), 7.04 (d, J = 7.6 Hz, 1H), |
| | 6.96 (s, 1H), 6.94-6.89 (m, 2H), 5.07-4.99 (m, 3H), 4.41 (d, J = 17.2 Hz, |
| | 1H), 4.25 (d, J = 16.8 Hz, 1H), 3.89-3.72 (m, 4H), 3.53 (s, 4H), 3.26 (s, 3H), |
| | 3.13-3.07 (m, 4H), 2.94-2.87 (m, 1H), 2.86-2.77 (m, 2H), 2.65-2.59 (m, |
| | 1H), 2.56-2.55 (m, 2H), 2.55 (s, 3H), 2.41 (dd, J = 4.4, 13.2 Hz, 1H), 2.21- |
| | 2.10 (m, 2H), 2.06-1.93 (m, 4H), 1.89-1.83 (m, 2H), 1.47-1.33 (m, 2H), |
| | 0.97-0.93 (m, 2H), 0.91-0.87 (m, 2H). |
| 192 | ¹H NMR (400MHz, DMSO-d₆) δ = 11.05 (s, 1 H), 8.17 (d, J = 1.2 Hz, 2 H), |
| | 7.85 ( d, J = 8.0 Hz, 1 H), 7.43 (s, 1 H), 7.34 (t, J = 8.0 Hz, 1 H), 6.87-6.97 (m, |
| | 4 H), 6.82 ( d, J = 7.5 Hz, 1 H), 6.58-6.68 (m, 1 H), 5.27 (m, 1 H), 4.95 (s, 2 |
| | H), 4.59 (m, 1 H), 3.70 (s, 3 H), 3.56 (d, J = 11.6 Hz, 2 H), 3.30 (s, 2 H), 3.25 |
| | (d, J = 7.6 Hz, 2 H), 2.88 (d, J = 8.4 Hz, 4 H), 2.79 (s, 3 H), 2.58-2.68 (m, 4 H), |
| | 2.52-2.55 (m, 3 H), 2.30-2.38 (m, 3 H), 2.13-2.21 (m, 2 H), 1.95-2.02 (m, |
| | 1 H), 1.73-1.85 (m, 6 H), 1.60-1.68 (m, 1 H), 1.45 (d, J = 6.8 Hz, 6 H), 1.20- |
| | 1.32 (m, 2 H). |
| 193 | ¹H NMR (400 MHz, DMSO-d6) δ: 10.95 (s, 1H), 8.18 (d, J = 6.0 Hz, 2H), |
| | 7.97 (s, 1H), 7.92 (s, 1H), 7.88 (d, J = 8.8 Hz, 1H), 7.41-7.31 (m, 3H), 7.07 |
| | (d, J = 8.4 Hz, 1H), 6.76 (d, J = 8.0 Hz, 1H), 5.12-5.02 (m, 3H), 4.96 (d, J = |
| | 6.0 Hz, 2H), 4.89 (d, J = 6.0 Hz, 2H), 4.43 (d, J = 16.8 Hz, 1H), 4.25 (d, J = |
| | 16.8 Hz, 1H), 3.85 (s, 3H), 3.68 (s, 2H), 3.57 (d, J = 8.8 Hz, 1H), 3.51 (s, 2H), |
| | 3.39-3.27 (m, 3H), 2.92-2.77 (m, 6H), 2.62-2.54 (m, 1H), 2.46-2.26 (m, |
| | 9H), 2.07 (d, J = 6.8 Hz, 2H), 2.00-1.88 (m, 5H), 1.75 (d, J = 11.2 Hz, 2H), |
| | 1.64-1.53 (m, 2H), 1.47-1.35 (m, 1H), 1.21-1.07 (m, 2H), 0.93-0.79 (m, |
| | 2H). |
| 194 | ¹H NMR (400 MHz, DMSO-d6) δ = 10.98 (s, 1H), 8.19 (s, 1H), 8.01 (s, 1H), |
| | 7.94 (s, 1H), 7.89 (dd, J = 1.6, 8.0 Hz, 1H), 7.47 (d, J = 8.4 Hz, 1H), 7.40 (s, |
| | 1H), 7.36 (t, J = 8.0 Hz, 1H), 7.17 (t, J = 8.0 Hz, 1H), 6.79 (d, J = 8.0 Hz, 1H), |
| | 5.11 (s, 2H), 5.09-5.01 (m, 1H), 4.97 (d, J = 6.0 Hz, 2H), 4.89 (d, J = 6.0 Hz, |
| | 2H), 4.48 (d, J = 16.8 Hz, 1H), 4.31 (d, J = 16.8 Hz, 1H), 4.25-3.93 (m, 1H), |
| | 3.68-3.57 (m, 1H), 3.52 (s, 2H), 3.49-3.41 (m, 1H), 3.40-3.36 (m, 2H), |
| | 3.31 (s, 4H), 2.99-2.93 (m, 2H), 2.92 (s, 3H), 2.91-2.86 (m, 1H), 2.63-2.59 |
| | (m, 1H), 2.59-2.55 (m, 1H), 2.42 (dd, J = 4.4, 12.8 Hz, 2H), 2.33-2.25 (m, |
| | 1H), 2.24-2.04 (m, 2H), 2.02-1.86 (m, 6H), 1.77 (d, J = 11.6 Hz, 2H), 1.60- |
| | 1.53 (m, 2H), 1.52-1.31 (m, 1H), 1.26-1.14 (m, 2H), 1.12 (s, 3H), 0.91 (d, |
| | J = 11.6 Hz, 2H) |
| 195 | ¹H NMR (400 MHz, DMSO-d6) δ: 10.98 (s, 1H), 8.17-8.23 (m, 2H), 7.99 (s, |
| | 1H), 7.92 (s, 1H), 7.89 (br d, J = 9.2 Hz, 1H), 7.46 (d, J = 8.0 Hz, 1H), 7.39 (s, |
| | 1H), 7.36 (t, J = 8.0 Hz, 1H), 7.17 (t, J = 8.0 Hz, 1H), 6.77 (d, J = 8.0 Hz, 1H), |
| | 5.03-5.14 (m, 3H), 4.97 (d, J = 6.0 Hz, 2H), 4.89 (d, J = 6.0 Hz, 2H), 4.41- |
| | 4.56 (m, 1H), 4.31 (d, J = 16.8 Hz, 1H), 4.03-4.20 (m, 1H), 3.61 (br d, J = 4.0 |
| | Hz, 1H), 3.52 (s, 3H), 3.45 (br d, J = 3.6 Hz, 2H), 2.89-2.98 (m, 6H), 2.60- |
| | 2.66 (m, 2H), 2.40-2.46 (m, 4H), 2.20 (br d, J = 10.0 Hz, 1H), 1.98-2.06 (m, |
| | 4H), 1.89-1.97 (m, 5H), 1.75-1.80 (m, 2H), 1.54-1.61 (m, 2H), 1.39-1.46 (m, |
| | 1H), 1.07-1.22 (m, 6H), 0.83-0.93 ppm (m, 2H). |
| 196 | ¹H NMR (400MHz, DMSO-d6) δ: 10.98 (s, 1H), 8.19 (s, 2H), 7.98 (s, 1H), |
| | 7.93 (s, 1H), 7.90-7.86 (m, 1H), 7.46 (d J = 8.0 Hz, 1H), 7.40 (s, 1H), 7.36 (t, |
| | J = 7.6 Hz, 1H), 7.17 (t, J = 7.6 Hz, 1H), 6.77 (d J = 7.6 Hz, 1H), 5.10 (s, 2H), |
| | 5.08 ( s, 1H), 4.97 (d J = 6.0 Hz, 2H), 4.89 (d J = 6.0 Hz, 2H), 4.51-4.45 (m, |
| | 1H), 4.31 (d J = 16.4 Hz, 1H), 3.66 (d J = 2.8 Hz, 2H), 3.63-3.58 (m, 2H), |
| | 3.52 (s, 3H), 2.98-2.93 (m, 2H), 2.91 (s, 3H), 2.89-2.84 (m, 1H), 2.77 (d J = |
| | 11.2 Hz, 1H), 2.61 (d J = 2.8 Hz, 2H), 2.57 ( s, 2H), 2.55-2.53 (m, 3H), 2.44 |
| | (d J = 4.8 Hz, 1H), 2.26-2.21 (m, 1H), 2.16-2.11 (m, 1H), 1.99-1.85 (m, |
| | 8H), 1.71-1.65 (m, 1H), 1.61-1.53 (m, 2H), 1.41-1.34 (m, 1H), 1.20-1.10 |
| | (m, 2H), 0.93 (d J = 6.0 Hz, 3H), 0.85-0.76 (m, 1H) |
| 197 | ¹H NMR (400 MHz, DMSO-d6) δ: 13.34-13.81 (m, 1 H), 10.98 (s, 1 H), 8.00 |
| | (s, 1 H), 7.94 (s, 1 H), 7.76-7.84 (m, 1 H), 7.50-7.68 (m, 1 H), 7.46 (d, J = 8.0 |
| | Hz, 1 H), 7.34 (t, J = 8.0 Hz, 1 H), 7.17 (t, J = 8.0 Hz, 1 H), 6.87 (d, J = 4.0 Hz, 1 |
| | H), 5.04-5.20 (m, 3 H), 5.00 (d, J = 5.6 Hz, 2 H), 4.89 (d, J = 6.0 Hz, 2 H), 4.48 |
| | (d, J = 16.8 Hz, 1 H), 4.31 (d, J = 16.8 Hz, 1 H), 3.74 (s, 2 H), 3.58-3.65 (m, 1 |
| | H), 3.44 (d, J = 7.6 Hz, 4 H), 3.27-3.30 (m, 2 H), 2.86-2.97 (m, 3 H), 2.55- |
| | 2.63 (m, 4 H), 2.53-2.55 (m, 2 H), 2.36-2.44 (m, 4 H), 2.13-2.29 (m, 2 H), |
| | 1.88-2.01 (m, 5 H), 1.77 (d, J = 11.6 Hz, 2 H), 1.46-1.65 (m, 3 H), 1.09-1.22 |
| | (m, 2 H), 0.81-1.00 (m, 2 H). |
| 198 | ¹H NMR (400 MHz, DMSO-d6) δ: 10.86 (s, 1H), 8.67 (d, J = 8.4 Hz, 1H), |
| | 8.11-8.25 (m, 2H), 7.90-8.02 (m, 2H), 7.85 (dd, J = 8.0, 1.6 Hz, 1H), 7.39- |
| | 7.50 (m, 2H), 7.35 (t, J = 8.0 Hz, 1H), 7.23-7.32 (m, 2H), 7.10 (br d, J = 7.6 |
| | Hz, 1H), 6.83 (d, J = 7.6 Hz, 1H), 5.10 (s, 2H), 4.68-4.85 (m, 1H), 3.69 (s, |
| | 2H), 3.56 (br d, J = 12.4 Hz, 4H), 3.23 (br d, J = 9.2 Hz, 2H), 2.85-2.97 (m, |
| | 7H), 2.75-2.82 (m, 4H), 2.56 (br d, J = 4.0 Hz, 1H), 2.36-2.46 (m, 6H), 2.05- |
| | 2.19 (m, 4H), 1.84-1.96 (m, 5H), 1.75 (br d, J = 12.0 Hz, 2H), 1.36-1.57 (m, |
| | 4H), 1.10-1.19 (m, 2H), 0.86 ppm (q, J = 12.0 Hz, 2H) |
| 199 | ¹H NMR (400 MHz, DMSO-d6) δ: 10.96 (s, 1H), 8.17 (s, 1H), 8.13 (s, 1H), |
| | 7.98 (s, 1H), 7.93 (s, 1H), 7.85 (d, J = 8.4 Hz, 1H), 7.45 (s, 1H), 7.39-7.31 |
| | (m, 2H), 7.08 (d, J = 8.0 Hz, 1H), 6.83 (d, J = 7.6 Hz, 1H), 5.12-5.02 (m, |
| | 3H), 4.43 (d, J = 17.2 Hz, 1H), 4.25 (d, J = 17.2 Hz, 1H), 3.85 (s, 3H), 3.70 (s, |
| | 2H), 3.58 (d, J = 3.6 Hz, 1H), 3.28-3.19 (m, 5H), 2.92-2.80 (m, 7H), 2.78 (s, |
| | 3H), 2.60 (s, 1H), 2.55 (d, J = 7.2 Hz, 2H), 2.44 (s, 8H), 2.13 (d, J = 4.8 Hz, |
| | 2H), 1.99-1.88 (m, 5H), 1.75 (d, J = 11.2 Hz, 2H), 1.58 (q, J = 9.2 Hz, 2H), |
| | 1.49-1.38 (m, 1H), 1.20-1.08 (m, 2H), 0.94-0.80 (m, 2H). |
| 200 | ¹H NMR (400 MHz, DMSO-d6) δ = 10.85 (s, 1H), 8.94 (d, J = 8.0 Hz, 1H), |
| | 8.22-8.13 (m, 3H), 7.97 (s, 1H), 7.92 (s, 1H), 7.84 (d, J = 9.2 Hz, 1H), 7.48- |
| | 7.42 (m, 2H), 7.34 (t, J = 8.0 Hz, 1H), 6.99 (dd, J = 2.0, 5.6 Hz, 1H), 6.82 |
| | (d, J = 7.6 Hz, 1H), 5.10 (s, 2H), 4.80-4.68 (m, 1H), 3.78-3.62 (m, 6H), 3.27- |
| | 3.10 (m, 7H), 2.88 (d, J = 7.6 Hz, 4H), 2.78 (s, 4H), 2.41 (s, 5H), 2.22-2.10 |
| | (m, 2H), 2.07-1.99 (m, 3H), 1.96-1.72 (m, 7H), 1.46-1.37 (m, 3H), 1.16- |
| | 1.07 (m, 2H), 0.90-0.78 (m, 2H). |
| 201 | ¹H NMR (400 MHz, dimethylsulfoxide-d6) δ = 10.78-10.82 (1 H, m), 8.20 (1 |
| | H, s), 8.17 (1 H, s), 7.97-7.99 (1 H, m), 7.91-7.94 (1 H, m), 7.83-7.89 (1 |
| | H, m), 7.46 (1 H, s), 7.35 (1 H, s), 7.06 (1 H, t, J = 8.4 Hz), 6.84 (1 H, s), 6.68- |
| | 6.74 (2 H, m), 5.10 (2 H, s), 3.85-3.91 (1 H, m), 3.69 (2 H, s), 3.57-3.63 (1 |
| | H, m), 3.49-3.54 (2 H, m), 3.19-3.27 (2 H, m), 2.93 (1 H, s), 2.95 (1 H, s), |
| | 2.89 (3 H, s), 2.88 (2 H, s), 2.79 (3 H, s), 2.69-2.77 (1 H, m), 2.54-2.60 (2 |
| | H, m), 2.36-2.46 (6 H, m), 2.10-2.17 (1 H, m), 2.07 (2 H, d, J = 7.2 Hz), |
| | 1.96-2.01 (1 H, m), 1.93 (1 H, d, J = 2.4 Hz), 1.81-1.87 (2 H, m), 1.71-1.78 (2 H, |
| | m), 1.40-1.49 (3 H, m), 1.24 (2 H, s), 1.09-1.18 (2 H, m), 0.83-0.90 (2 H, |
| | m) |
| 202 | ¹H NMR (400 MHz, DMSO-d6) δ: 10.87 (s, 1H), 8.48 (br dd, J = 8.4, 2.8 Hz, |
| | 1H), 8.18 (s, 1H), 8.14 (s, 1H), 7.91-8.04 (m, 2H), 7.86 (br d, J = 7.6 Hz, 1H), |
| | 7.46 (s, 1H), 7.35 (t, J = 8.0 Hz, 1H), 7.06-7.19 (m, 3H), 6.84 (br d, J = 7.6 |
| | Hz, 1H), 5.11 (br s, 2H), 4.69-4.82 (m, 1H), 3.71 (br s, 2H), 3.54-3.61 (m, |
| | 1H), 3.42-3.48 (m, 2H), 3.33 (br s, 4H), 3.31 (br s, 3H), 3.22-3.27 (m, 1H), |
| | 2.88 (br d, J = 8.4 Hz, 5H), 2.81-2.85 (m, 1H), 2.79 (s, 3H), 2.54-2.57 (m, |
| | 2H), 2.47 (br d, J = 1.6 Hz, 4H), 2.15-2.22 (m, 2H), 2.10 (br dd, J = 12.8, 4.0 |
| | Hz, 1H), 1.98-2.04 (m, 1H), 1.84-1.96 (m, 4H), 1.76 (br d, J = 12.4 Hz, 2H), |
| | 1.39-1.57 (m, 3H), 1.06-1.22 (m, 2H), 0.79-0.97 ppm (m, 2H) |
| 203 | ¹H NMR (400 MHz, DMSO-d6) δ: 10.87 (d, J = 12.4 Hz, 1H), 8.19-8.12 (m, |
| | 2H), 7.98 (s, 1H), 7.93 (s, 1H), 7.85 (d, J = 9.2 Hz, 1H), 7.72-7.67 (m, 1H), |
| | 7.46 (s, 1H), 7.35 (t, J = 7.6 Hz, 1H), 6.91-6.86 (m, 1H), 6.84 (d, J = 8.0 Hz, |
| | 1H), 5.10 (s, 2H), 4.74-4.65 (m, 1H), 4.53-4.38 (m, 1H), 4.15-4.02 (m, |
| | 2H), 3.69 (s, 3H), 3.25 ( s, 2H), 2.89 (d, J = 8.0 Hz, 4H), 2.80 (s, 3H), 2.62- |
| | 2.57 (m, 1H), 2.55 (s, 2H), 2.53-2.52 (m, 2H), 2.45-2.41 (m, 7H), 2.09 (d, |
| | J = 6.8 Hz, 3H), 2.02-1.90 (m, 4H), 1.88-1.82 (m, 2H), 1.76 (d, J = 11.6 Hz, |
| | 2H), 1.48-1.31 (m, 7H), 1.20-1.08 (m, 2H), 0.93-0.81 (m, 2H) |
| 204 | ¹H NMR (400MHz, DMSO-d6) δ: 11.09-10.82 (m, 1H), 8.28 (s, 1H), 8.16 |
| | (s, 1H), 8.01-7.89 (m, 2H), 7.84 (d, J = 7.6 Hz, 1H), 7.49-7.41 (m, 2H), 7.34 |
| | (t, J = 7.6 Hz, 1H), 7.16 (d, J = 8.0 Hz, 1H), 6.87-6.78 (m, 1H), 5.17-5.00 |
| | (m, 3H), 4.53-4.42 (m, 1H), 4.35-4.25 (m, 1H), 4.15-4.04 (m, 1H), 3.66- |
| | 3.56 (m, 2H), 3.30 ( s, 7H), 3.27-3.16 (m, 5H), 2.97-2.92 (m, 2H), 2.88 (d, |
| | J = 7.6 Hz, 5H), 2.78 (s, 3H), 2.60 (s, 2H), 2.21-2.14 (m, 1H), 2.07-2.00 (m, |
| | 3H), 1.92 (d, J = 10.0 Hz, 5H), 1.75 (d, J = 10.0 Hz, 2H), 1.60-1.51 (m, 2H), |
| | 1.27-1.19 (m, 2H), 1.08 (d, J = 6.0 Hz, 3H), 0.91-0.83 (m, 2H). |
| 205 | ¹H NMR (400 MHz, DMSO-d6) δ = 10.97 (s, 1H), 8.18 (s, 1H), 7.98 (s, 1H), |
| | 7.93 (s, 1H), 7.86 (dd, J = 1.2, 8.0 Hz, 1H), 7.46 (s, 1H), 7.35 (t, J = 8.0 Hz, |
| | 1H), 6.90 (d, J = 7.6 Hz, 1H), 6.83 (d, J = 8.0 Hz, 1H), 5.11 (s, 2H), 5.03 (dd, |
| | J = 4.8, 13.2 Hz, 1H), 4.43-4.36 (m, 1H), 4.25-4.20 (m, 1H), 3.69 (s, 2H), |
| | 3.64-3.57 (m, 1H), 3.31 (s, 3H), 3.27-3.20 (m, 2H), 2.95 (s, 2H), 2.90-2.83 |
| | (m, 5H), 2.79 (s, 3H), 2.63-2.56 (m, 2H), 2.55-2.53 (m, 4H), 2.46-2.40 (m, |
| | 4H), 2.40-2.33 (m, 4H), 2.09-2.04 (m, 2H), 1.98-1.87 (m, 5H), 1.76 (d, J = |
| | 12.4 Hz, 2H), 1.60-1.51 (m, 2H), 1.46-1.37 (m, 1H), 1.20-1.08 (m, 2H), |
| | 0.93-0.80 (m, 2H). |
| 206 | ¹H NMR (400 MHz, DMSO-d6) δ = 10.76 (s, 1H), 8.26 (s, 1H), 8.17 (s, 1H), |
| | 7.98 (s, 1H), 7.93 (s, 1H), 7.85 (dd, J = 1.2, 8.0 Hz, 1H), 7.46 (s, 1H), 7.35 (t, |
| | J = 8.0 Hz, 1H), 7.03 (d, J = 8.4 Hz, 2H), 6.88 (d, J = 8.8 Hz, 2H), 6.83 (d, J = |
| | 7.6 Hz, 1H), 5.10 (s, 2H), 3.74-3.67 (m, 3H), 3.58-3.50 (m, 4H), 3.31 (s, |
| | 2H), 3.26-3.22 (m, 2H), 2.89 (s, 2H), 2.87 (s, 2H), 2.84 (s, 1H), 2.79 (s, 3H), |
| | 2.60-2.54 (m, 3H), 2.43 (dd, J = 4.0, 8.4 Hz, 4H), 2.35 (s, 2H), 2.16-1.98 |
| | (m, 5H), 1.94-1.84 (m, 4H), 1.75 (d, J = 11.6 Hz, 2H), 1.52-1.39 (m, 3H), |
| | 1.18-1.07 (m, 2H), 0.92-0.80 (m, 2H). |
| 207 | ¹H NMR (400 MHz, DMSO-d6) δ = 10.96 (s, 1H), 9.01 (s, 1H), 8.20 (s, 1H), |
| | 8.15 (s, 1H), 7.93 (d, J = 8.0 Hz, 1H), 7.76 (s, 1H), 7.71 (d, J = 8.8 Hz, 1H), |
| | 7.56 (s, 1H), 7.52-7.42 (m, 2H), 7.17 (t, J = 8.0 Hz, 1H), 7.09 (d, J = 8.8 Hz, |
| | 1H), 7.01 (d, J = 8.0 Hz, 1H), 5.07 (dd, J = 4.8, 13.2 Hz, 1H), 5.02-4.85 (m, |
| | 4H), 4.56-4.25 (m, 2H), 3.67-3.60 (m, 1H), 3.58 (d, J = 2.8 Hz, 4H), 3.34 (s, |
| | 3H), 3.02 (s, 3H), 2.99-2.87 (m, 3H), 2.63-2.55 (m, 2H), 2.44 (d, J = 4.0 Hz, |
| | 8H), 2.12 (d, J = 6.8 Hz, 2H), 2.02-1.87 (m, 5H), 1.77 (d, J = 11.6 Hz, 2H), |
| | 1.62-1.50 (m, 2H), 1.45 (d, J = 6.8 Hz, 1H), 1.21-1.07 (m, 2H), 0.97-0.80 |
| | (m, 2H) |
| 208 | ¹H NMR (400 MHz, DMSO-d6) δ: 10.93-10.82 (m, 1H), 8.16 (s, 2H), 8.00- |
| | 7.88 (m, 2H), 7.83 (d, J = 6.8 Hz, 1H), 7.69 (d, J = 4.4 Hz, 1H), 7.44 (s, 1H), |
| | 7.34 (s, 1H), 6.92-6.77 (m, 2H), 5.14-5.00 (m, 2H), 4.72-4.63 (m, 1H), |
| | 4.53-4.33 (m, 2H), 4.09 (d, J = 10.4 Hz, 4H), 3.74-3.64 (m, 4H), 3.17 ( s, |
| | 2H), 2.90-2.74 (m, 9H), 2.61-2.58 (m, 2H), 2.17 (d, J = 6.4 Hz, 1H), 2.09- |
| | 1.97 (m, 6H), 1.96-1.70 (m, 8H), 1.38 (d, J = 0.8 Hz, 6H), 1.23 (m, 1H), |
| | 1.15-1.05 (m, 4H), 0.91-0.79 (m, 2H). |
| 209 | ¹H NMR (400 MHz, DMSO-d6) δ: 10.86-11.00 (m, 1H), 8.08-8.22 (m, 2H), |
| | 7.90-8.02 (m, 2H), 7.77 (br d, J = 8.0 Hz, 1H), 7.67-7.73 (m, 1H), 7.27-7.37 |
| | (m, 2H), 6.84-6.94 (m, 1H), 6.62-6.75 (m, 1H), 5.08 (br s, 2H), 4.63-4.78 (m, |
| | 1H), 4.39-4.55 (m, 1H), 4.01-4.21 (m, 3H), 3.63-3.79 (m, 1H), 3.38-3.49 (m, |
| | 2H), 3.19 (s, 3H), 2.70 (s, 3H), 2.57-2.63 (m, 4H), 2.13-2.25 (m, 5H), 1.90- |
| | 2.02 (m, 6H), 1.82-1.89 (m, 4H), 1.72-1.80 (m, 3H), 1.32-1.47 (m, 7H), 1.03- |
| | 1.22 (m, 7H), 0.81-1.00 (m, 3H). |
| 210 | ¹H NMR (400 MHz, DMSO-d6) δ = 10.80 (s, 1H), 8.21 (s, 1H), 8.17 (s, 1H), |
| | 7.98 (s, 1H), 7.95-7.90 (m, 2H), 7.85 (dd, J = 1.2, 8.4 Hz, 1H), 7.46 (s, 1H), |
| | 7.39-7.32 (m, 2H), 6.82 (dd, J = 8.4, 12.4 Hz, 2H), 5.10 (s, 2H), 3.99-3.92 |
| | (m, 2H), 3.75-3.68 (m, 3H), 3.66-3.61 (m, 1H), 3.23-3.23 (m, 1H), 3.28- |
| | 3.21 (m, 3H), 3.11-3.05 (m, 2H), 2.89 (s, 2H), 2.87 (s, 2H), 2.79 (s, 3H), |
| | 2.65-2.57 (m, 2H), 2.47-2.42 (m, 4H), 2.38 (d, J = 1.6 Hz, 2H), 2.24-2.13 (m, |
| | 2H), 2.07 (d, J = 6.4 Hz, 2H), 2.02-1.87 (m, 4H), 1.84-1.73 (m, 4H), 1.44- |
| | 1.37 (m, 2H), 1.36-1.31 (m, 1H), 1.17-1.08 (m, 2H), 0.91-0.81 (m, 2H). |
| 211 | ¹H NMR (400 MHz, DMSO-d6) δ = 10.99-10.83 (m, 1H), 8.23 (s, 1H), 8.14 |
| | (s, 1H), 7.99 (s, 1H), 7.93 (s, 1H), 7.87-7.80 (m, 1H), 7.74-7.66 (m, 1H), |
| | 7.55 (s, 1H), 7.34 (t, J = 8.0 Hz, 1H), 6.93-6.78 (m, 2H), 5.11 (s, 2H), 5.02 |
| | (d, J = 6.0 Hz, 2H), 4.92 (d, J = 6.0 Hz, 2H), 4.70 (dd, J = 5.2, 12.8 Hz, 1H), |
| | 4.52-4.38 (m, 1H), 4.16-4.03 (m, 2H), 3.72 (s, 3H), 3.63 (s, 2H), 3.30-3.23 |
| | (m, 4H), 2.89-2.66 (m, 2H), 2.59 (dd, J = 2.8, 7.6 Hz, 2H), 2.55 (d, J = 7.6 |
| | Hz, 2H), 2.49-2.42 (m, 4H), 2.38 (dd, J = 4.0, 7.2 Hz, 2H), 2.21 (dd, J = 2.8, |
| | 4.4 Hz, 2H), 1.99-1.89 (m, 3H), 1.85 (d, J = 10.0 Hz, 2H), 1.76 (d, J = 11.6 |
| | Hz, 2H), 1.48-1.34 (m, 6H), 1.14 (d, J = 12.0 Hz, 2H), 0.94-0.83 (m, 2H), |
| | 0.82-0.74 (m, 2H), 0.68-0.59 (m, 2H) |
| 212 | ¹H NMR (400 MHz, DMSO-d6) δ: 10.81-10.96 (m, 1H), 8.25 (s, 1H), 8.17 (s, |
| | 1H), 7.89-8.01 (m, 2H), 7.78 (br d, J = 8.0 Hz, 1H), 7.73 (s, 1H), 7.66-7.72 |
| | (m, 1H), 7.32 (t, J = 8.0 Hz, 1H), 7.05 (d, J = 8.0 Hz, 1H), 6.86-6.92 (m, 1H), |
| | 5.14 (s, 2H), 4.65-4.75 (m, 1H), 4.38-4.52 (m, 1H), 4.03-4.16 (m, 3H), 3.71 |
| | (br t, J = 8.0 Hz, 1H), 3.44 (br s, 3H), 3.25 (s, 4H), 3.11 (s, 3H), 2.76-2.85 (m, |
| | 1H), 2.54-2.69 (m, 6H), 2.15-2.23 (m, 1H), 1.97-2.08 (m, 4H), 1.90-1.95 (m, |
| | 2H), 1.82-1.88 (m, 2H), 1.72-1.80 (m, 2H), 1.38 (br dd, J = 14.2, 6.8 Hz, 6H), |
| | 1.13 (br d, J = 12.0 Hz, 2H), 1.09 (d, J = 6.0 Hz, 3H), 0.84-0.98 ppm (m, 6H) |
| 213 | ¹H NMR (400 MHz, DMSO-d6) δ: 10.79 (s, 1 H), 8.19 (d, J = 2.8 Hz, 1 H), |
| | 8.18 (s, 1 H), 7.98 (s, 1 H), 7.93 (s, 1 H), 7.82-7.89 (m, 1 H), 7.46 (s, 1 H), |
| | 7.26-7.40 (m, 2 H), 7.15 (d, J = 8.8 Hz, 1 H), 6.83 (d, J = 7.6 Hz, 1 H), 5.11 (s, |
| | 2 H), 3.88 (dd, J = 8.4, 5.2 Hz, 1 H), 3.70 (s, 2 H), 3.57-3.64 (m, 1 H), 3.49- |
| | 3.56 (m, 2 H), 3.31 (s, 2 H), 3.28-3.30 (m, 1 H), 3.20-3.28 (m, 2 H), 2.90- |
| | 2.97 (m, 2 H), 2.89 (s, 2 H), 2.84-2.88 (m, 2 H), 2.78 (s, 3 H), 2.56-2.62 (m, |
| | 1 H), 2.55 (d, J = 2.4 Hz, 1 H), 2.35-2.48 (m, 6 H), 2.22-2.33 (m, 1 H), 2.15- |
| | 2.21 (m, 1 H), 2.00-2.14 (m, 3 H), 1.81-1.96 (m, 4 H), 1.70-1.80 (m, 2 H), |
| | 1.38-1.57 (m, 3 H), 1.05-1.19 (m, 2 H), 0.79-0.94 (m, 2 H). |
| 214 | ¹H NMR (400 MHz, DMSO-d6) δ = 11.01-10.80 (m, 1H), 8.17 (d, J = 2.0 |
| | Hz, 2H), 8.03 (s, 1H), 7.97 (s, 1H), 7.91-7.81 (m, 1H), 7.70 (d, J = 8.8 Hz, |
| | 1H), 7.46 (s, 1H), 7.35 (t, J = 8.0 Hz, 1H), 6.94-6.86 (m, 1H), 6.84 (d, J = 7.6 |
| | Hz, 1H), 5.10 (s, 2H), 4.70 (dd, J = 5.2, 12.4 Hz, 1H), 4.48-4.34 (m, 1H), |
| | 4.14-4.02 (m, 2H), 3.97-3.82 (m, 2H), 3.76-3.69 (m, 1H), 3.38 (s, 4H), |
| | 3.35 (s, 3H), 3.32 (s, 4H), 2.91-2.85 (m, 4H), 2.84-2.81 (m, 1H), 2.79 (s, |
| | 3H), 2.74-2.68 (m, 2H), 2.63-2.58 (m, 2H), 2.39-2.33 (m, 1H), 2.01-1.91 |
| | (m, 3H), 1.90-1.79 (m, 4H), 1.78-1.72 (m, 1H), 1.72-1.64 (m, 1H), 1.45- |
| | 1.30 (m, 6H), 1.24-1.11 (m, 2H), 1.00-0.86 (m, 2H). |
| 215 | ¹H NMR (400 MHz, DMSO-d6) δ = 11.05 (s, 1H), 8.17 (s, 1H), 8.10-7.89 |
| | (m, 2H), 7.85 (d, J = 8.8 Hz, 1H), 7.45 (s, 1H), 7.36 (t, J = 8.0 Hz, 1H), 6.94- |
| | 6.80 (m, 3H), 6.66-6.58 (m, 1H), 5.33-5.24 (m, 1H), 5.12 (s, 2H), 3.80 (s, |
| | 2H), 3.65-3.36 (m, 6H), 3.30 (s, 3H), 3.28-3.16 (m, 2H), 3.12-2.93 (m, |
| | 4H), 2.90-2.87 (m, 4H), 2.85 (s, 1H), 2.80 (s, 3H), 2.79-2.56 (m, 4H), 2.54 |
| | (s, 1H), 2.48-2.37 (m, 2H), 2.16-1.83 (m, 6H), 1.82-1.74 (m, 2H), 1.73- |
| | 1.36 (m, 4H), 1.27-1.12 (m, 2H), 1.10-0.86 (m, 2H). |
| 216 | ¹H NMR (400 MHz, DMSO-d6) δ: 10.83-10.94 (m, 1H), 9.24 (br s, 1H), 8.20 |
| | (s, 1H), 8.14 (s, 1H), 8.02-8.07 (m, 1H), 7.90-7.99 (m, 1H), 7.86-7.89 (m, 1H), |
| | 7.67-7.72 (m, 1H), 7.47-7.57 (m, 2H), 7.02-7.11 (m, 1H), 6.84-6.93 (m, 1H), |
| | 4.96-5.01 (m, 4H), 4.66-4.74 (m, 1H), 4.37-4.45 (m, 1H), 3.99-4.18 (m, 3H), |
| | 3.68-3.73 (m, 2H), 3.56-3.62 (m, 3H), 3.35-3.38 (m, 4H), 3.03 (s, 3H), 2.56- |
| | 2.62 (m, 6H), 2.40 (br d, J = 6.0 Hz, 2H), 1.90-2.00 (m, 5H), 1.82-1.86 (m, |
| | 2H), 1.74-1.80 (m, 3H), 1.39 (br dd, J = 13.8, 6.4 Hz, 6H), 1.12-1.19 (m, 2H), |
| | 0.87-0.97 ppm (m, 2H) |
| 217 | ¹H NMR (400 MHz, DMSO-d6) δ = 10.93-10.83 (m, 1H), 8.17 (s, 1H), 8.02 |
| | (s, 1H), 7.96 (s, 1H), 7.85 (dd, J = 1.2, 8.0 Hz, 1H), 7.72-7.67 (m, 1H), 7.47 |
| | (s, 1H), 7.35 (t, J = 8.0 Hz, 1H), 6.92-6.86 (m, 1H), 6.84 (d, J = 7.6 Hz, 1H), |
| | 5.10 (s, 2H), 4.73-4.69 (m, 1H), 4.52-4.39 (m, 1H), 4.16-4.03 (m, 2H), |
| | 3.94-3.80 (m, 2H), 3.75-3.69 (m, 1H), 3.39-3.33 (m, 2H), 3.29-3.20 (m, |
| | 4H), 2.90 (s, 2H), 2.88 (s, 2H), 2.80 (s, 3H), 2.70-2.61 (m, 4H), 2.61-2.54 |
| | (m, 4H), 2.39 (dd, J = 6.8, 11.6 Hz, 1H), 2.24 (dd, J = 7.2, 12.0 Hz, 1H), |
| | 2.00-1.90 (m, 3H), 1.89-1.79 (m, 4H), 1.68 (d, J = 8.4 Hz, 1H), 1.60 (d, J = 8.4 |
| | Hz, 1H), 1.39 (dd, J = 6.8, 14.4 Hz, 6H), 1.27-1.19 (m, 1H), 1.19-1.07 (m, |
| | 2H), 0.96-0.84 (m, 2H). |
| 218 | ¹H NMR (400 MHz, DMSO-d6) δ: 10.78-10.99 (m, 1H), 8.25 (s, 1H), 7.92- |
| | 8.09 (m, 2H), 7.66-7.82 (m, 3H), 7.32 (br t, J = 8.0 Hz, 1H), 7.06 (br d, J = 7.2 |
| | Hz, 1H), 6.90 (br d, J = 9.2 Hz, 1H), 5.15 (br s, 2H), 4.61-4.79 (m, 1H), 4.35- |
| | 4.55 (m, 1H), 4.03-4.16 (m, 2H), 3.84-4.02 (m, 2H), 3.73 (br s, 1H), 3.55-3.70 |
| | (m, 1H), 3.41-3.47 (m, 1H), 3.35-3.40 (m, 2H), 3.28-3.30 (m, 1H), 3.25-3.28 |
| | (m, 3H), 3.12 (s, 2H), 2.96-3.08 (m, 1H), 2.85-2.92 (m, 1H), 2.76-2.84 (m, |
| | 2H), 2.64-2.71 (m, 2H), 2.54-2.63 (m, 3H), 1.96 (br d, J = 6.0 Hz, 3H), 1.76- |
| | 1.91 (m, 6H), 1.33-1.49 (m, 6H), 1.10-1.22 (m, 2H), 0.85-1.01 ppm (m, 6H) |
| 219 | ¹H NMR (400 MHz, DMSO-d6) δ: 11.04 (s, 1 H), 8.17 (s, 1 H), 7.99-8.06 |
| | (m, 1 H), 7.97 (s, 1 H), 7.82-7.87 (m, 1 H), 7.46 (s, 1 H), 7.36 (t, J = 8.0 Hz, 1 |
| | H), 6.98 (s, 1 H), 6.84-6.93 (m, 1 H), 6.72-6.84 (m, 2 H), 5.15-5.23 (m, 1 |
| | H), 5.12 (s, 2 H), 3.70-3.87 (m, 2 H), 3.51-3.59 (m, 3 H), 3.41-3.50 (m, 1 |
| | H), 3.38 (s, 1 H), 3.35-3.37 (m, 1 H), 3.34 (d, J = 1.6 Hz, 1 H), 3.27-3.30 (m, |
| | 1 H), 3.25 (d, J = 8.0 Hz, 1 H), 2.93-3.12 (m, 3 H), 2.90 (s, 3 H), 2.88 (s, 2 H), |
| | 2.84 (d, J = 3.2 Hz, 1 H), 2.81 (s, 3 H), 2.73-2.79 (m, 2 H), 2.60 (d, J = 4.4 Hz, |
| | 2 H), 2.56 (d, J = 8.4 Hz, 2 H), 2.35-2.48 (m, 3 H), 2.01-2.19 (m, 1 H), 1.83- |
| | 1.96 (m, 5 H), 1.63-1.82 (m, 3 H), 1.45-1.54 (m, 2 H), 1.09-1.21 (m, 2 H), |
| | 0.85-1.07 (m, 2 H). |
| 220 | ¹H NMR (400 MHz, DMSO-d6) δ = 10.96 (s, 1H), 8.17 (s, 1H), 8.14 (s, 1H), |
| | 8.01 (s, 1H), 7.94 (s, 1H), 7.85 (d, J = 8.4 Hz, 1H), 7.46 (s, 1H), 7.36 (t, J = |
| | 8.0 Hz, 1H), 6.90 (d, J = 8.0 Hz, 1H), 6.85 (d, J = 8.0 Hz, 1H), 5.11 (s, 2H), |
| | 5.03 (dd, J = 5.2, 13.2 Hz, 1H), 4.47-4.35 (m, 1H), 4.24 (d, J = 16.8 Hz, 1H), |
| | 4.18-4.03 (m, 1H), 3.66-3.58 (m, 1H), 3.50-3.41 (m, 2H), 3.40-3.36 (m, |
| | 4H), 3.27-3.12 (m, 4H), 2.95 (s, 2H), 2.90 (s, 3H), 2.88 (s, 2H), 2.80 (s, 3H), |
| | 2.65-2.59 (m, 2H), 2.57 (d, J = 1.0 Hz, 1H), 2.53 (s, 3H), 2.46-2.37 (m, 2H), |
| | 2.32-2.19 (m, 2H), 2.02-1.84 (m, 6H), 1.80-1.73 (m, 2H), 1.61-1.47 (m, |
| | 3H), 1.17 (s, 2H), 1.12 (d, J = 4.0 Hz, 3H), 1.00-0.85 (m, 2H) |
| 221 | ¹H NMR (400 MHz, DMSO-d6) δ: 10.98 (s, 1H), 9.59-9.85 (m, 1H), 9.38 (s, |
| | 1H), 8.90-9.20 (m, 1H), 8.24 (br s, 2H), 8.07 (br d, J = 8.0 Hz, 1H), 7.92 (br s, |
| | 1H), 7.76 (br s, 1H), 7.54 (t, J = 8.0 Hz, 1H), 7.47 (d, J = 8.0 Hz, 1H), 7.12- |
| | 7.22 (m, 2H), 5.08 (br dd, J = 13.2, 4.8 Hz, 1H), 4.99 (s, 4H), 4.40-4.68 (m, |
| | 3H), 4.31 (br d, J = 16.8 Hz, 1H), 3.68-3.94 (m, 1H), 3.61 (s, 3H), 3.46-3.51 |
| | (m, 2H), 3.08 (s, 3H), 2.84-3.03 (m, 10H), 2.55-2.71 (m, 4H), 2.27-2.46 (m, |
| | 5H), 1.84-2.12 (m, 8H), 1.67-1.82 (m, 4H), 1.51-1.65 (m, 3H), 0.86-1.26 (m, |
| | 6H). |
| 222 | ¹H NMR (400 MHz, DMSO-d6) δ: 10.88 (d, J = 12.8 Hz, 1 H), 8.25 (s, 1 H), |
| | 8.04 (s, 1 H), 7.97 (s, 1 H), 7.78 (d, J = 8.0 Hz, 1 H), 7.74 (s, 1 H), 7.70 (dd, |
| | J = 8.8, 2.8 Hz, 1 H), 7.32 (t, J = 8.0 Hz, 1 H), 7.06 (d, J = 7.2 Hz, 1 H), 6.89 (dd, |
| | J = 8.8, 2.8 Hz, 1 H), 5.14 (s, 2 H), 4.62-4.79 (m, 1 H), 4.37-4.54 (m, 1 H), |
| | 4.02-4.15 (m, 2 H), 3.93-4.02 (m, 1 H), 3.84-3.92 (m, 1 H), 3.70-3.76 (m, |
| | 1 H), 3.50-3.63 (m, 1 H), 3.40-3.43 (m, 1 H), 3.36 (s, 1 H), 3.26 (s, 3 H), |
| | 3.12 (s, 2 H), 2.93-3.04 (m, 1 H), 2.79-2.88 (m, 2 H), 2.78 (s, 1 H), 2.63- |
| | 2.69 (m, 2 H), 2.57-2.62 (m, 2 H), 2.54 (s, 2 H), 1.89-2.06 (m, 4 H), 1.73- |
| | 1.89 (m, 6 H), 1.43-1.49 (m, 1 H), 1.41 (d, J = 6.8 Hz, 3 H), 1.37 (d, J = 6.8 Hz, |
| | 2 H), 1.10-1.20 (m, 2 H), 0.92-1.03 (m, 4 H), 0.87-0.92 (m, 2 H). |
| 223 | ¹H NMR (400 MHz, DMSO-d6) δ = 10.91 (d, J = 13.2 Hz, 1H), 8.16-8.15 |
| | (m, 1H), 8.14 (s, 1H), 8.03 (s, 1H), 7.96 (s, 1H), 7.81-7.74 (m, 1H), 7.70 (dd, |
| | J = 3.2, 8.8 Hz, 1H), 7.35 (s, 1H), 7.31 (t, J = 8.0 Hz, 1H), 6.89 (dd, J = 4.0, |
| | 9.2 Hz, 1H), 6.68 (d, J = 8.0 Hz, 1H), 5.08 (s, 2H), 4.70 (dd, J = 5.2, 12.4 Hz, |
| | 1H), 4.55-4.37 (m, 1H), 4.17-4.04 (m, 2H), 4.01-3.85 (m, 2H), 3.77-3.69 |
| | (m, 1H), 3.62-3.52 (m, 1H), 3.32-3.26 (m, 4H), 3.19 (s, 3H), 3.04-2.91 (m, |
| | 1H), 2.87-2.75 (m, 3H), 2.70 (s, 3H), 2.66-2.53 (m, 6H), 2.37-2.31 (m, |
| | 2H), 2.22-2.13 (m, 1H), 2.00-1.91 (m, 3H), 1.90-1.79 (m, 6H), 1.79-1.72 |
| | (m, 1H), 1.38 (dd, J = 6.8, 14.4 Hz, 6H), 1.22-1.10 (m, 2H), 1.01-0.88 (m, |
| | 2H) |
| 224 | ¹H NMR (400 MHz, DMSO-d6) δ = 10.96 (s, 1H), 8.13 (s, 1H), 8.00 (s, 1H), |
| | 7.92 (s, 1H), 7.78-7.73 (m, 1H), 7.36 (s, 1H), 7.30 (t, J = 8.0 Hz, 1H), 6.90 |
| | (d, J = 7.6 Hz, 1H), 6.68 (d, J = 7.6 Hz, 1H), 5.13-4.98 (m, 3H), 4.50-4.35 |
| | (m, 1H), 4.24 (d, J = 16.8 Hz, 1H), 3.73-3.57 (m, 2H), 3.47-3.35 (m, 4H), |
| | 3.19 (s, 2H), 2.96-2.85 (m, 3H), 2.71 (s, 3H), 2.63-2.55 (m, 2H), 2.54 (s, |
| | 4H), 2.49-2.44 (m, 1H), 2.43-2.29 (m, 6H), 2.23-2.10 (m, 3H), 2.02 (d, J = |
| | 7.6 Hz, 2H), 1.98-1.85 (m, 6H), 1.79 (d, J = 11.6 Hz, 2H), 1.60-1.52 (m, |
| | 2H), 1.48-1.38 (m, 1H), 1.17 (s, 2H), 1.12 (s, 6H), 0.95-0.85 (m, 2H) |
| 225 | ¹H NMR (400 MHz, DMSO-d6) δ = 10.88 (d, J = 12.4 Hz, 1H), 8.16 (s, 1H), |
| | 8.13 (s, 1H), 8.03 (s, 1H), 7.96 (s, 1H), 7.76 (d, J = 9.2 Hz, 1H), 7.70 (dd, J = |
| | 2.8, 8.8 Hz, 1H), 7.44-7.22 (m, 2H), 6.89 (dd, J = 3.6, 8.8 Hz, 1H), 6.70 (d, |
| | J = 7.6 Hz, 1H), 5.07 (s, 2H), 4.70 (dd, J = 4.8, 12.4 Hz, 1H), 4.52-4.39 (m, |
| | 1H), 4.09 (dd, J = 15.2, 16.4 Hz, 2H), 3.98-3.86 (m, 2H), 3.74-3.70 (m, |
| | 1H), 3.61 (s, 1H), 3.36 (d, J = 3.6 Hz, 2H), 3.34 (d, J = 2.8 Hz, 2H), 3.19 (s, |
| | 3H), 3.04-2.98 (m, 1H), 2.86 (d, J = 9.6 Hz, 1H), 2.80 (d, J = 10.4 Hz, 2H), |
| | 2.72 (s, 3H), 2.66 (d, J = 8.4 Hz, 2H), 2.62-2.53 (m, 4H), 2.39-2.30 (m, 2H), |
| | 2.22-2.12 (m, 1H), 1.96 (dd, J = 4.0, 6.4 Hz, 3H), 1.91-1.73 (m, 7H), 1.39 |
| | (dd, J = 6.8, 14.4 Hz, 6H), 1.22-1.09 (m, 2H), 1.03-0.88 (m, 2H) |
| 226 | ¹H NMR (400 MHz, DMSO-d6) δ: 11.03 (s, 1 H), 8.07-8.32 (m, 1 H), 7.98 |
| | (s, 1 H), 7.93 (s, 1 H), 7.86 (d, J = 8.8 Hz, 1 H), 7.46 (s, 1 H), 7.35 (t, J = 7.6 Hz, |
| | 1 H), 7.21-7.31 (m, 2 H), 7.03 (d, J = 1.2 Hz, 1 H), 6.87-6.93 (m, 1 H), 6.83 |
| | (d, J = 6.8 Hz, 1 H), 6.35 (d, J = 3.2 Hz, 1 H), 5.53 (dd, J = 12.8, 4.8 Hz, 1 H), |
| | 5.05-5.17 (m, 2 H), 3.69 (s, 2 H), 3.51-3.58 (m, 1 H), 3.35 (s, 3 H), 3.15- |
| | 3.28 (m, 2 H), 2.88 (d, J = 8.4 Hz, 5 H), 2.79 (s, 3 H), 2.76 (s, 1 H), 2.70-2.75 |
| | (m, 1 H), 2.68 (s, 1 H), 2.54-2.63 (m, 2 H), 2.41 (d, J = 9.6 Hz, 4 H), 2.35- |
| | 2.39 (m, 2 H), 2.27-2.32 (m, 1 H), 2.04-2.12 (m, 3 H), 1.86-1.95 (m, 4 H), |
| | 1.73-1.80 (m, 2 H), 1.53-1.61 (m, 2 H), 1.24-1.47 (m, 2 H), 1.08-1.19 (m, |
| | 2 H), 0.81-0.93 (m, 2 H). |
| 227 | ¹H NMR (400 MHz, DMSO-d6) δ = 10.96 (s, 1H), 8.25 (s, 1H), 8.01 (s, 1H), |
| | 7.92 (s, 1H), 7.84-7.68 (m, 2H), 7.32 (t, J = 8.0 Hz, 1H), 7.05 (d, J = 7.6 Hz, |
| | 1H), 6.90 (d, J = 7.6 Hz, 1H), 5.13 (s, 2H), 5.03 (dd, J = 5.2, 13.2 Hz, 1H), |
| | 4.50-4.33 (m, 1H), 4.24 (d, J = 16.8 Hz, 1H), 3.61 (s, 1H), 3.37 (s, 2H), |
| | 3.32-3.28 (m, 4H), 3.25 (s, 3H), 3.12 (s, 2H), 2.97-2.86 (m, 3H), 2.59 (d, J = |
| | 16.8 Hz, 2H), 2.53 (s, 3H), 2.44-2.33 (m, 3H), 2.28-2.13 (m, 2H), 2.07- |
| | 1.86 (m, 7H), 1.79 (d, J = 10.8 Hz, 2H), 1.56 (d, J = 8.8 Hz, 2H), 1.49-1.35 |
| | (m, 1H), 1.30-1.05 (m, 8H), 0.99-0.79 (m, 6H) |
| 228 | ¹H NMR (400 MHz, DMSO-d6) δ: 11.08 (s, 1 H), 8.92-9.09 (m, 1 H), 8.24 |
| | (s, 1 H), 8.05 (s, 1 H), 7.99 (s, 1 H), 7.86 (d, J = 8.8 Hz, 1 H), 7.46 (s, 1 H), |
| | 7.37 (t, J = 8.0 Hz, 1 H), 6.95-7.02 (m, 1 H), 6.89 (d, J = 7.6 Hz, 1 H), 6.65- |
| | 6.86 (m, 1 H), 5.24-5.37 (m, 1 H), 5.14 (s, 2 H), 4.55-4.66 (m, 1 H), 3.86- |
| | 4.04 (m, 1 H), 3.82 (s, 2 H), 3.59 (d, J = 5.2 Hz, 3 H), 3.36-3.39 (m, 2 H), 3.33 |
| | (s, 3 H), 3.19-3.28 (m, 2 H), 3.00-3.10 (m, 3 H), 2.92-2.99 (m, 4 H), 2.89 |
| | (d, J = 7.6 Hz, 5 H), 2.84 (d, J = 2.4 Hz, 1 H), 2.82 (s, 3 H), 2.62-2.65 (m, 1 H), |
| | 2.59 (s, 1 H), 1.96 (d, J = 9.6 Hz, 5 H), 1.76-1.83 (m, 2 H), 1.66-1.74 (m, 1 |
| | H), 1.54-1.65 (m, 2 H), 1.45 (d, J = 6.8 Hz, 6 H), 1.13-1.24 (m, 2 H), 0.95- |
| | 1.09 (m, 2 H). |
| 229 | ¹H NMR (400 MHz, DMSO-d₆) δ: 11.08 (s, 1 H), 8.59-8.84 (m, 1 H), 8.22 |
| | (s, 1 H), 8.05 (s, 1 H), 7.95 (s, 1 H), 7.84 (d, J = 7.6 Hz, 1 H), 7.46 (s, 1 H), |
| | 7.34-7.41 (m, 1 H), 6.93-6.99 (m, 1 H), 6.89 (d, J = 7.2 Hz, 1 H), 6.58-6.84 |
| | (m, 1 H), 5.23-5.38 (m, 1 H), 5.04-5.23 (m, 2 H), 4.60 (t, J = 14.0, 6.8 Hz, 1 |
| | H), 4.04-4.29 (m, 1 H), 3.56-3.62 (m, 2 H), 3.32 (s, 3 H), 3.20-3.27 (m, 2 |
| | H), 2.99-3.10 (m, 2 H), 2.91-2.96 (m, 2 H), 2.90 (s, 3 H), 2.88 (s, 3 H), 2.82 |
| | (s, 3 H), 2.77-2.81 (m, 1 H), 2.70-2.76 (m, 1 H), 2.64 (d, J = 4.8 Hz, 1 H), |
| | 2.59 (d, J = 3.2 Hz, 1 H), 2.05-2.15 (m, 1 H), 1.82-2.04 (m, 6 H), 1.69-1.81 |
| | (m, 3 H), 1.53-1.67 (m, 3 H), 1.50 (s, 1 H), 1.45 (d, J = 6.8 Hz, 6 H), 1.21- |
| | 1.33 (m, 6 H), 1.16-1.21 (m, 1 H), 1.00-1.14 (m, 2 H), 0.71-0.98 (m, 1 H). |
| 230 | ¹H NMR (400 MHz, DMSO-d6) δ: 11.05 (s, 1 H), 8.17 (s, 1 H), 7.95 (s, 1 H), |
| | 7.89 (s, 1 H), 7.85 (d, J = 7.6 Hz, 1 H), 7.45 (s, 1 H), 7.35 (t, J = 8.0 Hz, 1 H), |
| | 6.87-6.95 (m, 2 H), 6.83 (d, J = 7.6 Hz, 1 H), 6.62 (d, J = 10.4 Hz, 1 H), 5.26 |
| | (dd, J = 12.4, 5.2 Hz, 1 H), 5.09 (s, 2 H), 4.59 (t, J = 14.0, 6.8 Hz, 1 H), 3.83- |
| | 4.12 (m, 2 H), 3.52-3.59 (m, 1 H), 3.37-3.43 (m, 3 H), 3.30 (s, 4 H), 3.22- |
| | 3.27 (m, 1 H), 2.88 (d, J = 7.6 Hz, 4 H), 2.83 (d, J = 2.4 Hz, 1 H), 2.79 (s, 3 H), |
| | 2.70 (d, J = 2.4 Hz, 2 H), 2.39 (s, 3 H), 2.09-2.13 (m, 2 H), 1.85-2.00 (m, 6 |
| | H), 1.75-1.81 (m, 2 H), 1.49-1.60 (m, 3 H), 1.45 (d, J = 6.8 Hz, 6 H), 1.07- |
| | 1.23 (m, 3 H), 0.79-0.99 (m, 3 H), 0.65 (s, 2 H), 0.47 (s, 2 H). |
| 231 | ¹H NMR (400 MHz, DMSO-d₆) δ: 11.05 (s, 1 H), 8.17 (s, 1 H), 8.14 (s, 1 H), |
| | 7.99 (s, 1 H), 7.93 (s, 1 H), 7.85 (dd, J = 8.0, 1.2 Hz, 1 H), 7.46 (s, 1 H), 7.35 (t, |
| | J = 8.0 Hz, 1 H), 6.88-6.94 (m, 2 H), 6.84 (d, J = 7.6 Hz, 1 H), 6.62 (dd, J = 8.8, |
| | 2.0 Hz, 1 H), 5.26 (d, J = 12.4 Hz, 1 H), 5.11 (s, 2 H), 4.54-4.63 (m, 1 H), 3.93 |
| | (s, 1 H), 3.71 (s, 2 H), 3.39 (t, J = 8.8, 4.4 Hz, 5 H), 3.31 (s, 3 H), 3.21-3.27 |
| | (m, 3 H), 2.90 (s, 2 H), 2.88 (s, 2 H), 2.85 (d, J = 5.6 Hz, 1 H), 2.80 (s, 3 H), |
| | 2.78 (s, 1 H), 2.76 (s, 1 H), 2.65-2.72 (m, 1 H), 2.61-2.64 (m, 1 H), 2.47 (s, |
| | 4 H), 2.36 (d, J = 6.8 Hz, 2 H), 1.93-2.03 (m, 3 H), 1.85-1.92 (m, 2 H), 1.47- |
| | 1.60 (m, 4 H), 1.44 (d, J = 6.8 Hz, 6 H). |
| 232 | ¹H NMR (400 MHz, DMSO-d6) δ: 10.88 (d, J = 12.4 Hz, 1H), 8.20 (s, 1H), |
| | 8.13 (s, 1H), 7.97 (s, 1H), 7.92 (s, 1H), 7.77 (dd, J = 1.6, 8.0 Hz, 1H), 7.69 |
| | (dd, J = 3.2, 8.8 Hz, 1H), 7.38-7.27 (m, 2H), 6.88 (dd, J = 3.6, 9.2 Hz, 1H), |
| | 6.68 (d, J = 8.0 Hz, 1H), 5.07 (s, 2H), 4.74-4.64 (m, 1H), 4.53-4.38 (m, 1H), |
| | 4.20-3.99 (m, 2H), 3.79-3.62 (m, 3H), 3.19 (s, 3H), 2.70 (s, 3H), 2.47-2.28 |
| | (m, 14H), 2.23-2.11 (m, 2H), 2.07 (d, J = 6.8 Hz, 2H), 2.00-1.71 (m, 9H), |
| | 1.41-1.37(m, 6H), 1.22-1.05 (m, 3H), 0.99-0.73 (m, 2H). |
| 233 | ¹H NMR (400 MHz, DMSO-d6) δ: 10.92-10.84 (m, 1H), 8.22 (s, 1H), 8.13 (s, |
| | 1H), 7.97 (s, 1H), 7.91 (s, 1H), 7.78 (br d, J = 8.0 Hz, 1H), 7.69 (dd, J = 3.2, |
| | 8.8 Hz, 1H), 7.63 (s, 1H), 7.35 (t, J = 8.0 Hz, 1H), 6.95 (br d, J = 7.2 Hz, 1H), |
| | 6.88 (dd, J = 3.6, 8.8 Hz, 1H), 5.10 (s, 2H), 4.74-4.64 (m, 1H), 4.53-4.36 |
| | (m, 1H), 4.18-4.01 (m, 2H), 3.87-3.65 (m, 6H), 3.54-3.41 (m, 5H), 3.03 (s, |
| | 2H), 2.75 (s, 3H), 2.61-2.57 (m, 3H), 2.38 (br s, 7H), 2.22-2.17 (m, 2H), |
| | 2.10-2.04 (m, 4H), 2.02-1.96 (m, 1H), 1.94-1.89 (m, 2H), 1.87-1.81 (m, |
| | 2H), 1.78-1.72 (m, 2H), 1.38 (br dd, J = 6.8, 14.4 Hz, 6H), 1.17-1.09 (m, |
| | 2H), 0.91-0.81 (m, 2H) |
| 234 | ¹H NMR (400 MHz, DMSO-d6) δ: 10.88 (br d, J = 12.8 Hz, 1H), 8.24 (s, 1H), |
| | 8.17 (s, 1H), 7.97 (s, 1H), 7.91 (s, 1H), 7.77 (br d, J = 8.8 Hz, 1H), 7.73 (br s, |
| | 1H), 7.69 (br dd, J = 2.8, 8.8 Hz, 1H), 7.34-7.28 (m, 1H), 7.04 (br d, J = 7.6 |
| | Hz, 1H), 6.88 (br dd, J = 2.8, 8.8 Hz, 1H), 5.13 (br s, 2H), 4.73-4.65 (m, 1H), |
| | 4.51 (br s, 1H), 4.14-4.03 (m, 2H), 3.68 (br s, 5H), 3.24 (s, 2H), 3.11 (s, 2H), |
| | 2.87-2.70 (m, 2H), 2.69-2.62 (m, 1H), 2.58 (br dd, J = 2.4, 6.4 Hz, 2H), 2.42 |
| | (br s, 4H), 2.38-2.29 (m, 4H), 2.06 (br d, J = 6.0 Hz, 2H), 2.01-1.95 (m, |
| | 1H), 1.95-1.90 (m, 2H), 1.88-1.81 (m, 2H), 1.78-1.71 (m, 2H), 1.38 (br dd, |
| | J = 6.8, 14.4 Hz, 6H), 1.18-1.08 (m, 2H), 0.96-0.89 (m, 4H), 0.88-0.80 (m, |
| | 2H) |
| 235 | ¹H NMR (400 MHz, DMSO-d6) δ: 10.88 (d, J = 12.8 Hz, 1H), 8.29 (s, 1H), |
| | 8.15 (s, 1H), 7.97 (s, 1H), 7.92 (s, 1H), 7.85 (dd, J = 1.2, 8.4 Hz, 1H), 7.69 |
| | (dd, J = 3.2, 8.8 Hz, 1H), 7.44-7.39 (m, 1H), 7.34 (t, J = 8.0 Hz, 1H), 6.88 |
| | (dd, J = 3.6, 8.8 Hz, 1H), 6.83 (d, J = 7.6 Hz, 1H), 5.08 (s, 2H), 4.99 (d, J = 6.0 |
| | Hz, 2H), 4.89 (d, J = 6.0 Hz, 2H), 4.69 (br dd, J = 5.2, 12.4 Hz, 1H), 4.52- |
| | 4.37 (m, 1H), 4.15-4.02 (m, 2H), 3.74-3.67 (m, 3H), 3.53 (s, 2H), 3.37 (br s, |
| | 6H), 2.83-2.72 (m, 1H), 2.62-2.56 (m, 2H), 2.45-2.31 (m, 7H), 2.07 (br d, |
| | J = 5.6 Hz, 2H), 2.02-1.95 (m, 1H), 1.92 (br d, J = 9.6 Hz, 2H), 1.87-1.80 |
| | (m, 2H), 1.75 (br d, J = 11.2 Hz, 2H), 1.38 (br dd, J = 6.8, 14.4 Hz, 6H), 1.18- |
| | 1.07 (m, 2H), 0.97 (t, J = 7.2 Hz, 3H), 0.92-0.80 (m, 2H) |
| 236 | ¹H NMR (400 MHz, DMSO-d6) δ: 10.90 (br d, J = 12.8 Hz, 1H), 8.19 (s, 1H), |
| | 7.99 (s, 1H), 7.95 (s, 1H), 7.90-7.85 (m, 1H), 7.69 (dd, J = 3.2, 9.2 Hz, 1H), |
| | 7.41 (s, 1H), 7.35 (t, J = 7.6 Hz, 1H), 6.89 (br dd, J = 3.2, 8.4 Hz, 1H), 6.76 (br |
| | d, J = 7.6 Hz, 1H), 5.14-5.07 (m, 2H), 4.97 (br d, J = 5.6 Hz, 2H), 4.89 (br d, |
| | J = 6.0 Hz, 2H), 4.74-4.65 (m, 1H), 4.53-4.38 (m, 1H), 4.17-4.02 (m, 2H), |
| | 3.77-3.69 (m, 1H), 3.64 (br s, 2H), 3.55-3.49 (m, 2H), 3.29-3.28 (m, 1H), |
| | 2.90 (s, 3H), 2.62 (br s, 4H), 2.16-2.02 (m, 4H), 2.01-1.89 (m, 4H), 1.88- |
| | 1.69 (m, 5H), 1.38 (br dd, J = 6.8, 14.1 Hz, 6H), 1.34-1.21 (m, 2H), 1.20- |
| | 1.05 (m, 3H), 0.94 (s, 6H), 0.88-0.79 (m, 2H) |
| 237 | ¹H NMR (400 MHz, DMSO-d6) δ = 8.42 (s, 1H), 8.19 (s, 1H), 8.08 (s, 1H), |
| | 7.97 (s, 1H), 7.76 (dd, J = 8.0, 2.0 Hz, 1H), 7.52 (d, J = 8.0 Hz, 1H), 7.42 (t, |
| | J = 8.0 Hz, 1H), 7.37 (s, 1H), 7.18 (t, J = 8.0 Hz, 1H), 6.79 (d, J = 7.6 Hz, 1H), |
| | 5.15-5.03 (m, 7H), 4.57-4.42 (m, 2H), 3.85-3.74 (m, 2H), 3.72-3.61 (m, 3H), |
| | 3.54-3.36 (m, 4H), 3.24-3.13 (m, 1H), 3.06-2.83 (m, 10H), 2.81-2.74 (m, 1H), |
| | 2.71-2.32 (m, 4H), 2.22-2.12 (m, 1H), 2.11-1.92 (m, 5H), 1.88-1.78 (m, 1H), |
| | 1.77-1.63 (m, 3H), 1.39-1.23 (m, 5H), 1.21-1.01 (m, 2H). |
| 238 | ¹H NMR (400 MHz, DMSO-d6) δ: 8.18 (s, 1H), 7.97 (s, 1H), 7.92 (s, 1H), |
| | 7.87 (d, J = 8.4 Hz, 1H), 7.69 (d, J = 8.8 Hz, 1H), 7.39 (s, 1H), 7.35 (t, J = 8.0 |
| | Hz, 1H), 6.88 (d, J = 3.2 Hz, 1H), 6.76 (d, J = 7.6 Hz, 1H), 5.09 (s, 2H), 4.96 |
| | (d, J = 6.4 Hz, 2H), 4.88 (d, J = 6.4 Hz, 2H), 4.69 (d, J = 12.8 Hz, 1H), 4.52- |
| | 4.38 (m, 1H), 4.13-4.03 (m, 2H), 3.75-3.66 (m, 2H), 3.65 (d, J = 2.4 Hz, |
| | 2H), 3.51 (s, 3H), 2.89 (s, 3H), 2.84-2.66 (m, 3H), 2.61-2.53 (m, 4H), 2.46- |
| | 2.27 (m, 3H), 2.26-2.19 (m, 1H), 2.16-2.09 (m, 1H), 1.99-1.78 (m, 9H), |
| | 1.66 (d, J = 12.0 Hz, 1H), 1.38 (d, J = 6.8 Hz, 6H), 1.17-1.07 (m, 2H), 0.92 |
| | (d, J = 5.6 Hz, 3H), 0.75 (s, 1H) |
| 239 | ¹H NMR (400 MHz, DMSO-d₆) δ: 10.90 (d, J = 12.8 Hz, 1H), 8.17 (s, 1H), |
| | 8.14 (s, 1H), 7.98 (s, 1H), 7.92 (s, 1H), 7.84 (br d, J = 8.0 Hz, 1H), 7.69 (dd, |
| | J = 3.2, 8.8 Hz, 1H), 7.45 (s, 1H), 7.34 (t, J = 8.0 Hz, 1H), 6.88 (dd, J = 3.6, 8.8 |
| | Hz, 1H), 6.82 (d, J = 7.6 Hz, 1H), 5.10 (s, 2H), 4.73-4.66 (m, 1H), 4.52- |
| | 4.37 (m, 1H), 4.16-4.03 (m, 3H), 3.70 (td, J = 4.0, 8.0 Hz, 1H), 3.42 (br d, |
| | J = 14.4 Hz, 3H), 3.30 (br s, 3H), 3.28-3.19 (m, 3H), 2.88 (s, 2H), 2.87-2.85 |
| | (m, 2H), 2.82 (br d, J = 9.2 Hz, 1H), 2.77 (s, 3H), 2.66-2.60 (m, 1H), 2.60- |
| | 2.52 (m, 4H), 2.23-2.14 (m, 1H), 2.04 (br s, 2H), 2.01-1.96 (m, 1H), 1.95- |
| | 1.88 (m, 3H), 1.86-1.80 (m, 2H), 1.79-1.72 (m, 2H), 1.37 (br dd, J = 6.8, |
| | 14.4 Hz, 6H), 1.19-1.10 (m, 2H), 1.08 (d, J = 6.0 Hz, 3H), 0.92-0.80 (m, |
| | 2H) |
| 240 | ¹H NMR (400 MHz, DMSO-d₆) δ: 10.90 (d, J = 12.8 Hz, 1H), 8.17 (s, 2H), |
| | 7.98 (s, 1H), 7.92 (s, 1H), 7.85 (br d, J = 8.0 Hz, 1H), 7.69 (dd, J = 3.2, 8.8 |
| | Hz, 1H), 7.45 (s, 1H), 7.34 (t, J = 8.0 Hz, 1H), 6.88 (dd, J = 3.6, 8.8 Hz, 1H), |
| | 6.82 (d, J = 7.6 Hz, 1H), 5.10 (s, 2H), 4.74-4.64 (m, 1H), 4.53-4.37 (m, 1H), |
| | 4.16-4.02 (m, 3H), 3.74-3.66 (m, 1H), 3.43 (br d, J = 4.0 Hz, 3H), 3.30 (br |
| | s, 3H), 3.27-3.21 (m, 3H), 2.88 (s, 2H), 2.86 (br s, 2H), 2.82 (br d, J = 9.2 |
| | Hz, 1H), 2.77 (s, 3H), 2.65-2.60 (m, 1H), 2.59-2.52 (m, 4H), 2.17 (br t, J = |
| | 9.2 Hz, 1H), 2.02 (br d, J = 6.0 Hz, 2H), 2.00-1.96 (m, 1H), 1.96-1.88 (m, |
| | 3H), 1.87-1.82 (m, 2H), 1.75 (br d, J = 12.0 Hz, 2H), 1.37 (br dd, J = 6.8, |
| | 14.4 Hz, 6H), 1.19-1.10 (m, 2H), 1.08 (d, J = 6.0 Hz, 3H), 0.92-0.80 (m, |
| | 2H) |
| 241 | ¹H NMR (400 MHz, DMSO-d₆) δ: 10.97 (s, 1H), 8.18 (s, 1H), 7.98 (s, 1H), |
| | 7.91 (s, 1H), 7.90-7.86 (m, 1H), 7.46 (d, J = 8.1 Hz, 1H), 7.39 (s, 1H), 7.35 |
| | (t, J = 8.0 Hz, 1H), 7.16 (t, J = 8.1 Hz, 1H), 6.76 (d, J = 7.6 Hz, 1H), 5.12- |
| | 5.04 (m, 3H), 4.96 (d, J = 6.0 Hz, 2H), 4.88 (d, J = 6.1 Hz, 2H), 4.51-4.44 |
| | (m, 1H), 4.30 (d, J = 16.9 Hz, 1H), 4.14 (br d, J = 14.4 Hz, 1H), 3.65-3.58 |
| | (m, 1H), 3.51 (s, 2H), 3.36 (br d, J = 3.0 Hz, 4H), 2.98-2.92 (m, 2H), 2.90 (s, |
| | 3H), 2.84-2.79 (m, 1H), 2.62-2.55 (m, 2H), 2.45-2.36 (m, 3H), 2.19-2.10 |
| | (m, 1H), 2.00-1.88 (m, 6H), 1.82 (br s, 2H), 1.81-1.75 (m, 1H), 1.67-1.61 |
| | (m, 1H), 1.60-1.51 (m, 2H), 1.44-1.33 (m, 1H), 1.19 (br s, 1H), 1.17-1.10 |
| | (m, 2H), 1.08 (br d, J = 6.0 Hz, 3H), 0.94-0.75 (m, 5H) |
| 242 | ¹H NMR (400 MHz, MeOD) δ = 8.49 (s, 1H), 8.19 (s, 1H), 8.08 (s, 1H), 7.98 |
| | (s, 1H), 7.76 (dd, J = 8.4, 1.6 Hz, 1H), 7.52 (d, J = 8.4 Hz, 1H), 7.45-7.32 (m, |
| | 2H), 7.17 (t, J = 8.0 Hz, 1H), 6.78 (d, J = 8.0 Hz, 1H), 5.16-5.01 (m, 7H), |
| | 4.57-4.41 (m, 2H), 4.25-4.10 (m, 1H), 3.76-3.59 (m, 3H), 3.56-3.39 (m, 4H), |
| | 3.15-2.70 (m, 11H), 2.72-2.33 (m, 5H), 2.22-1.80 (m, 7H), 1.77-1.51 (m, 3H), |
| | 1.39-0.92 (m, 10H). |
| 243 | ¹H NMR (400 MHz, DMSO-d₆) δ: 10.89 (d, J = 12.8 Hz, 1H), 8.19 (s, 1H), |
| | 7.98 (s, 1H), 7.92 (s, 1H), 7.88 (dd, J = 1.6, 8.0 Hz, 1H), 7.69 (dd, J = 3.2, 8.8 |
| | Hz, 1H), 7.40 (s, 1H), 7.35 (t, J = 8.0 Hz, 1H), 6.88 (dd, J = 3.6, 9.2 Hz, 1H), |
| | 6.77 (d, J = 8.0 Hz, 1H), 5.10 (s, 2H), 4.96 (d, J = 6.0 Hz, 2H), 4.88 (d, J = 6.0 |
| | Hz, 2H), 4.73-4.66 (m,1 H), 4.51-4.38 (m, 1H), 4.14-4.03 (m, 2H), 3.73- |
| | 3.60 (m, 3H), 3.51 (s, 2H), 2.90 (s, 3H), 2.85-2.68 (m, 2H), 2.62-2.56 (m, |
| | 3H), 2.31-2.15 (m, 2H), 2.01-1.90 (m, 4H), 1.89-1.75 (m, 6H), 1.43-1.33 |
| | (m, 6H), 1.33-1.20 (m, 2H), 1.19-1.05 (m, 4H), 1.04-0.78 (m, 8H) |
| 244 | ¹H NMR (400 MHz, DMSO-d₆) δ: 10.97 (s, 1H), 8.18 (s, 1H), 7.97 (s, 1H), |
| | 7.92 (s, 1H), 7.89-7.86 (m, 1H), 7.45 (d, J = 8.4 Hz, 1H), 7.40 (s, 1H), 7.35 |
| | (t, J = 8.0 Hz, 1H), 7.16 (t, J = 8.0 Hz, 1H), 6.76 (d, J = 8.4 Hz, 1H), 5.09 (s, |
| | 2H), 5.08-5.03 (m, 1H), 4.96 (d, J = 6.0 Hz, 2H), 4.89 (d, J = 6.4 Hz, 2H), |
| | 4.50-4.45 (m, 1H), 4.33-4.28 (m, 1H), 3.69-3.63 (m, 2H), 3.62-3.58 (m, |
| | 1H), 3.51 (s, 3H), 2.98-2.93 (m, 2H), 2.90 (s, 4H), 2.82-2.68 (m, 2H), 2.65- |
| | 2.59 (m, 4H), 2.05-1.81 (m, 10H), 1.72-1.51 (m, 4H), 1.42-1.32 (m, 1H), |
| | 1.25-1.04 (m, 4H), 0.94-0.90 (m, 3H), 0.89-0.72 (m, 2H). |
| 245 | ¹H NMR (400 MHz, DMSO-d₆) δ = 11.06 (s, 1H), 8.17 (s, 1H), 7.98 (s, 1H), |
| | 7.92 (s, 1H), 7.87-7.83 (m, 1H), 7.45 (s, 1H), 7.34 (t, J = 8.0 Hz, 1H), 6.92- |
| | 6.88 (m, 2H), 6.82 (d, J = 7.6 Hz, 1H), 6.61 (dd, J = 1.6, 8.4 Hz, 1H), 5.26 (br |
| | dd, J = 5.2, 12.8 Hz, 1H), 5.10 (s, 2H), 4.62-4.54 (m, 1H), 4.10 (br d, J = |
| | 14.0 Hz, 1H), 3.56-3.50 (m, 1H), 3.39 (br d, J = 7.2 Hz, 3H), 3.30 (s, 3H), |
| | 3.28-3.17 (m, 2H), 2.87 (br d, J = 7.2 Hz, 5H), 2.77 (s, 4H), 2.65-2.55 (m, |
| | 4H), 2.17 (br t, J = 8.8 Hz, 1H), 2.06-1.96 (m, 4H), 1.90 (br t, J = 11.6 Hz, |
| | 6H), 1.78-1.72 (m, 2H), 1.53 (br d, J = 9.2 Hz, 2H), 1.43 (d, J = 6.8 Hz, 8H), |
| | 1.15-1.10 (m, 2H), 1.08 (d, J = 6.0 Hz, 3H), 0.89-0.80 (m, 2H) |
| 246 | ¹H NMR (400 MHz, MeOD) δ: 8.19 (s, 1H), 8.09 (s, 1H), 7.94 (s, 1H), 7.75 |
| | (d, J = 2.4 Hz, 2H), 7.44-7.35 (m, 2H), 6.84 (d, J = 2.4 Hz, 1H), 6.75 (d, J = |
| | 7.2 Hz, 1H), 5.10-5.05 (m, 6H), 4.87-4.85 (m, 1H), 4.73-4.64 (m, 1H), |
| | 4.59-4.45 (m, 1H), 4.26-4.16 (m, 2H), 3.82-3.78 (m, 1H), 3.66 (s, 2H), |
| | 3.49-3.34 (m, 4H), 2.89 (s, 3H), 2.81-2.74 (m, 2H), 2.73-2.64 (m, 1H), |
| | 2.47 (br s, 2H), 2.40-2.19 (m, 3H), 2.17-2.12 (m, 1H), 2.10-2.02 (m, 4H), |
| | 1.98-1.87 (m, 4H), 1.59-1.46 (m, 6H), 1.32-1.21 (m, 3H), 1.20 (s, 6H), |
| | 1.01-0.90 (m, 2H) |
| 247 | ¹H NMR (400 MHz, MeOD) δ: 8.18 (s, 1H), 8.08 (s, 1H), 7.96 (s, 1H), 7.76- |
| | 7.72 (m, 2H), 7.53 (s, 1H), 7.40 (t, 1H), 6.92 (d, J = 8.0 Hz, 1H), 6.84 (d, J = |
| | 2.4 Hz, 1H), 5.09 (s, 2H), 4.71-4.65 (m, 1H), 4.57-4.48 (m, 1H), 4.26 (d, J = |
| | 14.4 Hz, 1H), 4.20 (d, J = 4.0 Hz, 2H), 3.80 (d, J = 4.0, 7.6 Hz, 1H), 3.43 (s, |
| | 3H), 3.41 (d, J = 2.0 Hz, 1H), 3.38 (d, J = 3.2 Hz, 1H), 3.17 (d, J = 8.0 Hz, |
| | 1H), 3.01 (s, 2H), 3.00 (d, J = 3.6 Hz, 2H), 2.83 (s, 3H), 2.80 (s, 1H), 2.75 (d, |
| | J = 4.0 Hz, 2H), 2.73-2.67 (m, 2H), 2.63-2.58 (m, 1H), 2.32 (d, J = 10.6 Hz, |
| | 1H), 2.26-2.11 (m, 4H), 2.04 (t, J = 4.4 Hz, 3H), 1.97-1.83 (m, 5H), 1.58- |
| | 1.53 (m, 2H), 1.53-1.47 (m, 5H), 1.26 (d, J = 11.2 Hz, 2H), 1.20 (d, J = 6.4 |
| | Hz, 3H), 1.04-0.95 (m, 2H) |
| 248 | ¹H NMR (400 MHz, MeOD) δ = 8.18 (s, 1H), 8.08 (s, 1H), 7.95 (s, 1H), 7.74 |
| | (dd, J = 2.4, 8.8 Hz, 2H), 7.53 (s, 1H), 7.40 (t, J = 8.0 Hz, 1H), 6.91 (d, J = 8.0 |
| | Hz, 1H), 6.84 (dd, J = 2.4, 8.8 Hz, 1H), 5.09 (s, 2H), 4.72-4.64 (m, 1H), 4.57- |
| | 4.47 (m, 1H), 4.22 (br d, J = 10.0 Hz, 3H), 3.79 (td, J = 4.0, 8.0 Hz, 1H), |
| | 3.43 (s, 3H), 3.40 (br s, 1H), 3.37 (br d, J = 3.2 Hz, 1H), 3.35 (br d, J = 2.8 |
| | Hz, 1H), 3.19 (t, J = 8.4 Hz, 1H), 3.03-2.98 (m, 4H), 2.83 (s, 3H), 2.82-2.78 |
| | (m, 1H), 2.78-2.72 (m, 3H), 2.68 (br s, 1H), 2.66 (br d, J = 3.6 Hz, 1H), |
| | 2.60-2.54 (m, 1H), 2.33-2.26 (m, 1H), 2.17-2.10 (m, 4H), 2.04 (br s, 1H), 2.01 |
| | (br s, 1H), 1.98-1.91 (m, 3H), 1.89-1.83 (m, 2H), 1.57-1.54 (m, 1H), 1.50 |
| | (dd, J = 6.8, 13.2 Hz, 5H), 1.30-1.23 (m, 2H), 1.19 (d, J = 6.0 Hz, 3H), 1.03- |
| | 0.94 (m, 2H) |
| 249 | ¹H NMR (400 MHz, DMSO-d₆) δ: 8.19 (s, 1H), 8.05 (s, 1H), 7.96 (s, 1H), |
| | 7.78-7.72 (m, 2H), 7.42-7.36 (m, 2H), 6.84 (d, J = 2.4 Hz, 1H), 6.75 (d, J = |
| | 8.0 Hz, 1H), 5.09-5.05 (m, 6H), 4.73 (s, 2H), 4.56-4.48 (m, 1H), 4.24-4.17 |
| | (m, 2H), 3.80 (d, J = 3.6 Hz, 1H), 3.73-3.68 (m, 1H), 3.66 (s, 2H), 3.60-3.54 |
| | (m, 1H), 3.50-3.37 (m, 3H), 2.99-2.94 (m, 1H), 2.90 (s, 3H), 2.80-2.73 (m, |
| | 2H), 2.56-2.48 (m, 2H), 2.31-2.22 (m, 2H), 2.17-2.11 (m, 1H), 2.06-2.01 |
| | (m, 2H), 1.96-1.90 (m, 3H), 1.87-1.81 (m, 1H), 1.59-1.45 (m, 6H), 1.45- |
| | 1.38 (m, 1H), 1.34-1.17 (m, 3H), 1.10-1.02 (m, 6H), 1.01-0.82 (m, 3H) |
| 250 | ¹H NMR (400 MHz, DMSO-d₆) δ: 10.88 (d, J = 12.8 Hz, 1H), 8.18 (s, 1H), |
| | 7.96 (d, J = 16.4 Hz, 2H), 7.86 (d, J = 8.4 Hz, 1H), 7.69 (d, J = 3.2, 8.9 Hz, |
| | 1H), 7.41 (s, 1H), 7.35 (t, 1H), 6.88 (d, J = 3.2 Hz, 1H), 6.76 (d, J = 7.6 Hz, |
| | 1H), 5.10 (s, 2H), 4.96 (d, J = 6.0 Hz, 2H), 4.88 (d, J = 6.0 Hz, 2H), 4.73- |
| | 4.65 (m, 1H), 4.52-4.37 (m, 1H), 4.15-4.02 (m, 2H), 3.74-3.65 (m, 2H), |
| | 3.54 (d, J = 14.4 Hz, 1H), 3.51 (s, 2H), 2.90 (s, 3H), 2.82 (d, J = 2.4 Hz, 2H), |
| | 2.70-2.64 (m, 2H), 2.61-2.58 (m, 1H), 2.33 (d, J = 3.6 Hz, 2H), 2.31-2.27 |
| | (m, 1H), 2.20-2.06 (m, 3H), 2.02-1.86 (m, 4H), 1.86-1.71 (m, 4H), 1.42- |
| | 1.34 (m, 5H), 1.33-1.20 (m, 2H), 1.20-1.04 (m, 3H), 0.95 (d, J = 6.4 Hz, |
| | 6H), 0.89-0.76 (m, 2H) |
| 251 | ¹H NMR (400 MHz, CD₃OD) δ = 8.48 (s, 1H), 8.17 (s, 1H), 8.10 (s, 1H), 7.95 |
| | (s, 1H), 7.76-7.70 (m, 1H), 7.52 (s, 1H), 7.40 (t, J = 8.0 Hz, 1H), 6.95-6.85 (m, |
| | 2H), 5.11-5.00 (m, 3H), 4.48-4.35 (m, 2H), 3.71-3.65 (m, 1H), 3.50-3.41 (m, |
| | 5H), 3.21-3.15 (m, 1H), 3.07-2.92 (m, 7H), 2.92-2.72 (m, 6H), 2.58 (s, 3H), |
| | 2.56-2.31 (m, 6H), 2.24-2.09 (m, 3H), 2.08-1.97 (m, 4H), 1.94-1.86 (m, 2H), |
| | 1.75-1.64 (m, 2H), 1.59-1.45 (m, 1H), 1.35-1.15 (m, 9H), 1.05-0.93 (m, 2H). |
| 252 | ¹H NMR (400 MHz, MeOD) δ: 8.89 (s, 1H), 8.19 (s, 1H), 7.97-7.89 (m, 2H), |
| | 7.79-7.74 (m, 2H), 7.59-7.56 (m, 1H), 7.55-7.52 (m, 1H), 7.16 (d, J = 7.2 |
| | Hz, 1H), 6.86 (dd, J = 2.4, 9.2 Hz, 1H), 4.77-4.63 (m, 2H), 4.63-4.48 (m, |
| | 3H), 4.26-4.19 (m, 2H), 3.85-3.80 (m, 1H), 3.50 (m, 3H), 3.44-3.38 (m, |
| | 2H), 3.27-3.23 (m, 1H), 3.13-3.03 (m, 4H), 2.93 (s, 3H), 2.81-2.74 (m, |
| | 2H), 2.65-2.57 (m, 2H), 2.25-2.02 (m, 6H), 2.00-1.87 (m, 5H), 1.62-1.47 |
| | (m, 7H), 1.35-1.30 (m, 2H), 1.28 (s, 6H), 1.26-1.22 (m, 1H), 1.09-0.94 (m, |
| | 2H). |
| 253 | ¹H NMR (400MHz, DMSO-d₆) δ = 10.92-10.84 (m, 1H), 9.31-9.07 (m, |
| | 1H), 8.25 (s, 1H), 8.04 (s, 1H), 7.98 (br d, J = 8.4 Hz, 1H), 7.90 (s, 1H), 7.69 |
| | (dd, J = 2.8, 8.4 Hz, 1H), 7.51-7.42 (m, 2H), 7.30 (br d, J = 7.2 Hz, 1H), |
| | 6.91-6.85 (m, 1H), 4.74-4.65 (m, 1H), 4.52-4.38 (m, 1H), 4.16-4.02 (m, 2H), |
| | 3.74-3.68 (m, 1H), 3.31 (br s, 3H), 3.30-3.27 (m, 2H), 3.18 (s, 2H), 2.84- |
| | 2.73 (m, 1H), 2.58 (br d, J = 5.6 Hz, 1H), 2.54 (br s, 4H), 2.40 (br s, 2H), |
| | 2.29-2.08 (m, 3H), 2.05-1.97 (m, 3H), 1.95-1.90 (m, 2H), 1.88-1.76 (m, 4H), |
| | 1.44-1.35 (m, 6H), 1.27-1.17 (m, 2H), 1.13 (s, 7H), 0.98 (br d, J = 5.6 Hz, |
| | 4H), 0.93-0.83 (m, 2H). |
| 254 | ¹H NMR (400 MHz, DMSO-d₆) δ: 10.98 (brs, 1H), 8.17 (s, 1H), 8.00 (s, 1H), |
| | 7.92 (s, 1H), 7.83 (d, J = 8.4 Hz, 1H), 7.46 (s, 2H), 7.34 (t, J = 8.0 Hz, 1H), |
| | 7.16 (d, J = 7.6 Hz, 1H), 6.82 (d, J = 7.2 Hz, 1H), 5.25-5.03 (m, 3H), 4.53- |
| | 4.44 (m, 1H), 4.34-4.25 (m, 1H), 3.69-3.60 (m, 2H), 3.39-3.25 (m, 4H), 3.24- |
| | 3.17 (m, 2H), 2.99-2.83 (m, 8H), 2.78 (s, 3H), 2.62-2.53 (m, 2H), 2.48-2.31 |
| | (m, 3H), 2.29-2.10 (m, 3H), 2.05-1.98 (m, 2H), 1.97-1.87 (m, 5H), 1.78 (d, J = |
| | 10.4 Hz, 2H), 1.56 (d, J = 8.4 Hz, 2H), 1.50-1.38 (m, 1H), 1.31-1.13 (m, 2H), |
| | 1.11 (s, 6H), 0.94-0.78 (m, 2H). |
| 255 | ¹H NMR (400 MHz, DMSO-d₆) δ: 10.89 (d, J = 12.8 Hz, 1H), 9.17 (s, 1H), |
| | 8.14 (s, 1H), 7.97 (dd, J = 8.0, 1.6 Hz, 1H), 7.90 (s, 1H), 7.73 (s, 1H), 7.69 |
| | (dd, J = 8.8, 3.2 Hz, 1H), 7.49 (s, 1H), 7.42 (t, J = 8.0 Hz, 1H), 6.87 (d, J = 8.0 |
| | Hz, 2H), 4.69 (dd, J = 12.8, 4.8 Hz, 1H), 4.53-4.38 (m, 1H), 4.16-4.02 (m, |
| | 2H), 3.73-3.67 (m, 1H), 3.34-3.28 (m, 5H), 3.27 (s, 3H), 2.78 (s, 3H), 2.62- |
| | 2.53 (m, 4H), 2.45-2.36 (m, 4H), 2.24-2.12 (m, 3H), 2.03-1.88 (m, 6H), 1.88- |
| | 1.74 (m, 6H), 1.43-1.34 (m, 6H), 1.20-1.15 (m, 1H), 1.12 (s, 6H), 1.10-1.07 |
| | (m, 1H), 0.93-0.80 (m, 2H). |
| 256 | ¹H NMR (400 MHz, DMSO-d6) δ: 10.99 (s, 1 H), 9.24 (s, 1 H), 8.21 (s, 1 H), |
| | 8.15 (s, 2 H), 8.01-8.06 (m, 1 H), 7.90 (s, 2 H), 7.54 (s, 1 H), 7.45-7.52 (m, |
| | 2 H), 7.10-7.17 (m, 1 H), 7.04 (d, J = 7.6 Hz, 1 H), 5.08 (m, 1 H), 4.94-5.01 |
| | (m, 4 H), 4.49 (d, J = 17.2 Hz, 1 H), 4.26-4.36 (m, 1 H), 3.65-3.70 (m, 1 H), |
| | 3.57-3.63 (m, 3 H), 3.10 (s, 4 H), 3.02 (s, 3 H), 2.79-2.93 (m, 4 H), 2.69 (d, |
| | J = 11.2 Hz, 2 H), 2.61 (s, 1 H), 2.47 ( s, 4 H), 2.36-2.42 (m, 1 H), 2.18-2.30 |
| | (m, 2 H), 2.08-2.15 (m, 3 H), 1.88-2.03 (m, 3 H), 1.76-1.86 (m, 2 H), |
| | 1.60-1.70 (m, 4 H), 1.45-1.55 (m, 2 H), 0.93-1.11 (m, 3 H). |
| 257 | ¹H NMR (400 MHz, DMSO-d6) δ: 11.00 (s, 1 H), 9.23 (s, 1 H), 8.19 (s, 1 H), |
| | 8.16 (s, 2 H), 7.99-8.05 (m, 1 H), 7.89 (d, J = 8.8 Hz, 2 H), 7.53 (s, 1 H), 7.44 |
| | (d, J = 8.0 Hz, 2 H), 7.06 (t, J = 7.6 Hz, 1 H), 6.99 (d, J = 7.6 Hz, 1 H) 5.08 (dd, |
| | J = 13.6, 4.8 Hz, 1 H), 4.87-5.00 (m, 4 H), 4.40-4.51 (m, 1 H), 4.30 (d, |
| | J = 16.8 Hz, 1 H), 3.63 (s, 2 H), 3.57 (s, 2 H), 3.05 (s, 4 H), 2.99 (s, 3 H), 2.88- |
| | 2.94 (m, 1 H), 2.82 (d, J = 7.2 Hz, 1 H), 2.75 (d, J = 1.2 Hz, 2 H), 2.57-2.64 (m, |
| | 2 H), 2.44 (d, J = 4.8 Hz, 1 H), 2.34-2.41 (m, 4 H), 2.05-2.19 (m, 5 H), 1.95- |
| | 2.03 (m, 2 H), 1.75-1.85 (m, 2 H), 1.58-1.72 (m, 5 H), 1.48-1.57 (m, 2 H), |
| | 1.37-1.46 (m, 1 H), 0.87-1.00 (m, 2 H). |
| 258 | ¹H NMR (400 MHz, DMSO-d6) δ = 10.98 (s, 1H), 9.22 (s, 1H), 8.20 (s, 1H), |
| | 8.16 (s, 2H), 8.07-7.98 (m, 1H), 7.89 (d, J = 6.8 Hz, 2H), 7.54 (s, 1H), 7.48 |
| | (t, J = 8.0 Hz, 1H), 7.42 (d, J = 8.0 Hz, 1H), 7.10 (t, J = 7.6 Hz, 1H), 7.02 (d, |
| | J = 7.4 Hz, 1H), 5.07 (dd, J = 4.8, 13.6 Hz, 1H), 5.01-4.93 (m, 4H), 4.49-4.40 |
| | (m, 1H), 4.36-4.22 (m, 1H), 3.63 (d, J = 6.4 Hz, 2H), 3.60-3.50 (m, 4H), |
| | 3.01 (s, 3H), 2.93-2.82 (m, 4H), 2.82-2.72 (m, 2H), 2.67-2.54 (m, 2H), |
| | 2.46-2.35 (m, 2H), 2.19-2.10 (m, 2H), 2.09-2.02 (m, 1H), 2.00-1.94 (m, |
| | 1H), 1.89-1.73 (m, 7H), 1.68-1.58 (m, 3H), 1.55-1.46 (m, 3H), 1.33-1.24 |
| | (m, 1H), 1.16-0.83 (m, 3H). |
| 259 | ¹H NMR (400 MHz, DMSO-d6) δ = 10.89 (s, 1H), 9.13 (s, 1H), 8.10 (s, 1H), |
| | 8.06 (s, 1H), 7.94 (dd, J = 1.6, 8.0 Hz, 1H), 7.79 (d, J = 4.0 Hz, 2H), 7.44 (s, |
| | 1H), 7.42-7.33 (m, 2H), 7.05 (t, J = 8.0 Hz, 1H), 6.93 (d, J = 8.0 Hz, 1H), |
| | 4.97 (dd, J = 5.2, 13.2 Hz, 1H), 4.92-4.84 (m, 4H), 4.38 (d, J = 16.8 Hz, 1H), |
| | 4.21 (d, J = 16.8 Hz, 1H), 3.54 (d, J = 15.2 Hz, 3H), 3.49 (s, 4H), 2.91 (s, 3H), |
| | 2.87-2.74 (m, 4H), 2.73-2.67 (m, 1H), 2.62 (d, J = 10.8 Hz, 2H), 2.56-2.46 |
| | (m, 2H), 2.32 (dd, J = 4.4, 13.2 Hz, 1H), 2.18-2.05 (m, 2H), 2.04-1.92 (m, |
| | 3H), 1.90-1.85 (m, 1H), 1.78 (d, J = 9.6 Hz, 2H), 1.72-1.61 (m, 4H), 1.59- |
| | 1.49 (m, 2H), 1.48-1.37 (m, 3H), 1.32-1.17 (m, 2H), 0.91-0.76 (m, 1H). |
| 260 | ¹H NMR (400 MHz, DMSO-d6) δ = 10.99 (s, 1H), 8.84 (s, 1H), 8.20 (s, 1H), |
| | 8.16 (s, 1H), 7.87 (dd, J = 1.4, 8.0 Hz, 1H), 7.69 (s, 1H), 7.65 (d, J = 8.0 Hz, |
| | 1H), 7.58 (d, J = 9.2 Hz, 1H), 7.43 (t, J = 8.0 Hz, 1H), 7.32-7.23 (m, 1H), |
| | 6.91 (d, J = 7.8 Hz, 1H), 6.53 (dd, J = 1.6, 8.8 Hz, 1H), 6.33 (s, 1H), 5.11 (dd, |
| | J = 5.2, 13.2 Hz, 1H), 5.00-4.96 (m, 2H), 4.96-4.91 (m, 2H), 4.42 (d, J = |
| | 17.6 Hz, 1H), 4.26 (d, J = 17.6 Hz, 1H), 3.62 (s, 4H), 3.57 (s, 2H), 2.99 (s, |
| | 3H), 2.92-2.86 (m, 1H), 2.75 (t, J = 11.6 Hz, 2H), 2.59 (d, J = 15.6 Hz, 2H), |
| | 2.53 (s, 2H), 2.39 (d, J = 1.6 Hz, 3H), 2.23 (d, J = 6.8 Hz, 2H), 2.09-1.93 (m, |
| | 2H), 1.83 (s, 2H), 1.79 (s, 4H), 1.73 (d, J = 3.6 Hz, 1H), 1.38-1.26 (m, 2H) |
| 261 | ¹H NMR (400 MHz, DMSO-d6) δ = 10.98 (s, 1H), 8.87 (s, 1H), 8.20 (s, 1H), |
| | 7.87 (d, J = 7.6 Hz, 1H), 7.69 (s, 1H), 7.61 (d, J = 8.8 Hz, 1H), 7.44 (t, J = 8.0 |
| | Hz, 1H), 6.93 (dd, J = 8.0, 12.8 Hz, 2H), 6.53 (d, J = 9.2 Hz, 1H), 6.35 (s, |
| | 1H), 5.04 (dd, J = 4.8, 13.2 Hz, 1H), 5.00-4.91 (m, 4H), 4.38-4.44 (m, 1H), |
| | 4.22-4.28 (m, 1H), 3.80-3.63 (m, 4H), 3.61-3.54 (m, 3H), 3.53 (d, J = 2.8 |
| | Hz, 2H), 3.14-3.07 (m, 2H), 3.01 (s, 3H), 2.85-2.77 (m, 2H), 2.62 (s, 1H), |
| | 2.55 (s, 6H), 2.44-2.36 (m, 2H), 2.05-1.92 (m, 4H), 1.89-1.83 (m, 2H), |
| | 1.46-1.31 (m, 2H), 1.18 (t, J = 7.2 Hz, 2H). |
| 262 | ¹H NMR (400 MHz, DMSO-d6) δ = 11.00 (s, 1H), 9.06 (s, 1H), 8.19 (s, 1H), |
| | 8.15 (s, 1H), 7.98 (dd, J = 1.2, 8.0 Hz, 1H), 7.84 (s, 1H), 7.71-7.61 (m, 1H), |
| | 7.44 (t, J = 8.0 Hz, 1H), 7.27 (d, J = 8.4 Hz, 1H), 7.00 (d, J = 8.0 Hz, 1H), |
| | 6.92 (s, 1H), 6.63 (s, 1H), 5.11 (dd, J = 5.2, 13.2 Hz, 1H), 4.50-4.35 (m, 1H), |
| | 4.33-4.22 (m, 1H), 3.69 (s, 4H), 3.28 (d, J = 8.8 Hz, 6H), 3.01-2.90 (m, 5H), |
| | 2.88 (s, 3H), 2.75 (t, J = 11.2 Hz, 2H), 2.47 (d, J = 4.4 Hz, 1H), 2.44 (d, J = |
| | 4.4 Hz, 1H), 2.42-2.32 (m, 3H), 2.23 (d, J = 6.8 Hz, 2H), 2.03-1.96 (m, 1H), |
| | 1.88-1.76 (m, 6H), 1.73 (dd, J = 2.4, 5.6 Hz, 1H), 1.40-1.25 (m, 2H) |
| 263 | ¹H NMR (400 MHz, DMSO-d6) δ = 11.06 (s, 1H), 8.19 (s, 1H), 7.96 (s, 1H), |
| | 7.93-7.87 (m, 2H), 7.40 (s, 1H), 7.35 (t, J = 8.0 Hz, 1H), 6.95-6.84 (m, 2H), |
| | 6.76 (d, J = 8.4 Hz, 1H), 6.66-6.57 (m, 1H), 5.27 (s, 1H), 5.10 (s, 2H), 4.97 |
| | (d, J = 6.0 Hz, 2H), 4.89 (d, J = 6.0 Hz, 2H), 4.58 (t, J = 7.2 Hz, 1H), 3.73- |
| | 3.61 (m, 2H), 3.52 (s, 3H), 3.44-3.36 (m, 5H), 2.91 (s, 3H), 2.79 (t, J = 10.0 |
| | Hz, 3H), 2.66-2.60 (m, 3H), 2.56 (s, 5H), 2.46-2.38 (m, 2H), 2.00 (d, J = 6.8 |
| | Hz, 3H), 1.96-1.85 (m, 7H), 1.81-1.67 (m, 4H), 1.66-1.52 (m, 5H), 1.45 (s, |
| | 3H), 1.44 (s, 3H), 1.18-1.08 (m, 2H), 0.92 (s, 3H), 0.89 (s, 3H), 0.83 (d, J = |
| | 11.2 Hz, 2H) |
| 264 | ¹H NMR (400 MHz, DMSO-d₆) δ: 10.98 (s, 1 H), 8.25 (s, 1 H), 8.23-7.85 |
| | (m, 2 H), 7.82-7.77 (m, 1 H), 7.74 (s, 1 H), 7.47 (d, J = 8.0 Hz, 1 H), 7.36- |
| | 7.30 (m, 1 H), 7.17 (t, J = 8.0 Hz, 1 H), 7.08 (d, J = 8.0 Hz, 1 H), 5.28-5.12 |
| | (m, 2 H), 5.08 (dd, J = 13.6, 4.8 Hz, 1 H), 4.48 (d, J = 16.8 Hz, 2 H), 4.31 (d, |
| | J = 16.8 Hz, 1 H), 3.98-3.68 (m, 1 H), 3.62 (m, 1 H), 3.52-3.35 (m, 5 H), 3.28 |
| | (s, 3 H), 3.13 (s, 2 H), 3.00-2.83 (m, 5 H), 2.64-2.55 (m, 3 H), 2.47-2.36 |
| | (m, 3 H), 2.09-1.88 (m, 9 H), 1.81-1.67 (m, 4 H), 1.65-1.49 (m, 4 H), |
| | 1.46-1.23 (m, 4 H), 1.21-1.03 (m, 6 H), 1.00-0.80 (m, 8 H). |
| 265 | ¹H NMR (400 MHz, DMSO-d6) δ: 10.96 (s, 1H), 8.19 (s, 1H), 7.88 (dd, J = |
| | 1.2, 8.4 Hz, 1H), 7.42-7.29 (m, 3H), 7.08 (d, J = 8.4 Hz, 1H), 6.94 (d, J = 4.4 |
| | Hz, 2H), 6.75 (d, J = 8.0 Hz, 1H), 5.06 (dd, J = 5.2, 13.2 Hz, 1H), 4.99-4.91 |
| | (m, 4H), 4.88 (d, J = 6.0 Hz, 2H), 4.44 (d, J = 16.8 Hz, 1H), 4.27 (d, J = 16.8 |
| | Hz, 1H), 3.87 (s, 3H), 3.79-3.71 (m, 4H), 3.63-3.45 (m, 8H), 2.90 (s, 3H), |
| | 2.88-2.81 (m, 1H), 2.78-2.72 (m, 2H), 2.61-2.56 (m, 1H), 2.54-2.52 (m, |
| | 1H), 2.47-2.41 (m, 1H), 2.03-1.92 (m, 1H), 1.88-1.67 (m, 8H). |
| 266 | ¹H NMR (400 MHz, DMSO-d6) δ: 11.06 (s, 1H), 9.26 (br s, 1H), 8.21 (s, |
| | 1H), 8.12-8.17 (m, 1H), 8.05 (br d, J = 7.6 Hz, 1H), 7.87-7.96 (m, 2H), 7.56 |
| | (br s, 1H), 7.50 (br t, J = 8.0 Hz, 1H), 7.05 (br d, J = 7.2 Hz, 1H), 6.86-6.95 |
| | (m, 2H), 6.62 (br d, J = 8.8 Hz, 1H), 5.22-5.33 (m, 1H), 4.99 (s, 4H), 4.51- |
| | 4.66 (m, 1H), 3.63-3.87 (m, 2H), 3.60 (s, 2H), 3.50-3.57 (m, 1H), 3.38 (br d, |
| | J = 5.2 Hz, 3H), 3.03 (s, 3H), 2.73-2.94 (m, 5H), 2.57-2.70 (m, 3H), 2.07-2.30 |
| | (m, 4H), 1.83-2.03 (m, 9H), 1.60-1.81 (m, 6H), 1.49-1.59 (m, 3H), 1.45 (br d, |
| | J = 6.8 Hz, 6H), 1.31-1.42 (m, 2H), 1.05-1.28 (m, 3H), 0.90-1.05 (m, 7H), |
| | 0.77-0.89 ppm (m, 2H) |
| 267 | ¹H NMR (400 MHz, DMSO-d6) δ: 10.98 (s, 1H), 9.28 (br s, 1H), 8.21 (s, |
| | 1H), 8.14 (s, 1H), 8.03-8.08 (m, 1H), 7.92-8.02 (m, 1H), 7.90 (s, 1H), 7.55- |
| | 7.64 (m, 1H), 7.51 (t, J = 8.0 Hz, 1H), 7.47 (d, J = 8.0 Hz, 1H), 7.17 (t, J = 8.0 |
| | Hz, 1H), 7.08 (br d, J = 8.0 Hz, 1H), 5.08 (dd, J = 13.2, 5.2 Hz, 1H), 4.96-5.01 |
| | (m, 4H), 4.48 (d, J = 17.2 Hz, 1H), 4.31 (d, J = 16.8 Hz, 1H), 3.65-4.06 (m, |
| | 2H), 3.60 (s, 3H), 3.35-3.42 (m, 3H), 3.04 (s, 3H), 2.86-2.98 (m, 4H), 2.59 (br |
| | d, J = 16.4 Hz, 3H), 2.36-2.49 (m, 5H), 1.86-2.03 (m, 9H), 1.70 (br s, 5H), |
| | 1.47-1.62 (m, 4H), 1.21-1.45 (m, 2H), 1.08-1.19 (m, 3H), 0.92-1.07 (m, 6H), |
| | 0.76-0.91 ppm (m, 3H) |
| 268 | ¹H NMR (400 MHz, DMSO-d6) δ: 11.05 (s, 1H), 9.23 (s, 1H), 8.21 (s, 1H), |
| | 7.98-8.11 (m, 1H), 7.89 (br d, J = 3.6 Hz, 2H), 7.45-7.56 (m, 2H), 7.03 (d, J = |
| | 8.0 Hz, 1H), 6.85-6.95 (m, 2H), 6.57-6.67 (m, 1H), 5.26 (dd, J = 12.8, 5.6 Hz, |
| | 1H), 4.90-5.03 (m, 4H), 4.50-4.67 (m, 1H), 3.62-3.69 (m, 1H), 3.59 (s, 2H), |
| | 3.55 (br d, J = 4.8 Hz, 1H), 3.35-3.43 (m, 2H), 3.01 (s, 3H), 2.73-2.91 (m, |
| | 4H), 2.53-2.65 (m, 4H), 2.46 (br s, 1H), 2.11-2.31 (m, 8H), 1.99-2.09 (m, 3H), |
| | 1.96 (br d, J = 7.2 Hz, 2H), 1.84-1.93 (m, 4H), 1.66-1.83 (m, 5H), 1.49-1.64 |
| | (m, 4H), 1.44 (d, J = 6.8 Hz, 6H), 1.17-1.42 (m, 2H), 1.05-1.16 (m, 2H), 0.89- |
| | 1.01 (m, 1H), 0.73-0.87 ppm (m, 2H) |
| 269 | ¹H NMR (400 MHz, DMSO-d6) δ = 10.97 (s, 1H), 8.33-8.06 (m, 2H), 7.89 |
| | (d, J = 8.4 Hz, 1H), 7.45-7.33 (m, 2H), 6.92-6.87 (m, 1H), 6.86-6.79 (m, |
| | 1H), 5.17 (s, 2H), 5.05-4.95 (m, 3H), 4.89 (d, J = 6.0 Hz, 2H), 4.39 (d, J = |
| | 16.8 Hz, 1H), 4.23 (d, J = 17.2 Hz, 1H), 3.69-3.55 (m, 2H), 3.52 (s, 2H), 2.94 |
| | (s, 3H), 2.94-2.87 (m, 4H), 2.86 (d, J = 5.2 Hz, 1H), 2.73-2.64 (m, 2H), 2.63- |
| | 2.54 (m, 4H), 2.53 (s, 3H), 2.45 (s, 2H), 2.44-2.28 (m, 4H), 2.16-2.01 (m, |
| | 2H), 2.00-1.83 (m, 8H), 1.83-1.64 (m, 5H), 1.64-1.46 (m, 4H), 1.29 (s, |
| | 2H), 1.26-1.12 (m, 4H), 1.11-0.95 (m, 5H), 0.94-0.72 (m, 2H). |
| 270 | ¹H NMR (400 MHz, DMSO-d6) δ = 10.98 (s, 1H), 8.28-7.98 (m, 3H), 7.78 |
| | (d, J = 8.0 Hz, 1H), 7.47 (d, J = 8.0 Hz, 1H), 7.42-7.27 (m, 2H), 7.17 (t, J = |
| | 8.0 Hz, 1H), 6.74 (d, J = 7.2 Hz, 1H), 5.21-5.03 (m, 3H), 4.48 (d, J = 16.8 |
| | Hz, 2H), 4.31 (d, J = 16.8 Hz, 1H), 3.65-3.59 (m, 1H), 3.50-3.36 (m, 6H), |
| | 3.31-3.26 (m, 2H), 3.21 (s, 2H), 3.01-2.85 (m, 5H), 2.74 (s, 3H), 2.71-2.65 |
| | (m, 1H), 2.62 (d, J = 2.4 Hz, 2H), 2.56 (d, J = 9.2 Hz, 4H), 2.48-2.39 (m, |
| | 2H), 2.39-2.28 (m, 3H), 2.24-2.11 (m, 2H), 2.05-1.89 (m, 7H), 1.86 (dd, |
| | J = 4.4, 9.2 Hz, 2H), 1.79-1.69 (m, 3H), 1.64-1.53 (m, 3H), 1.49-1.18 (m, |
| | 4H), 1.18-1.03 (m, 6H), 0.98-0.73 (m, 2H) |
| 271 | ¹H NMR (400 MHz, DMSO-d6) δ = 10.97 (s, 1H), 8.17 (s, 1H), 8.15 (s, 1H), |
| | 7.84 (dd, J = 1.2, 8.4 Hz, 1H), 7.42 (s, 1H), 7.33 (t, J = 8.0 Hz, 1H), 6.91 (d, |
| | J = 2.8 Hz, 2H), 6.81 (d, J = 7.6 Hz, 1H), 6.60 (d, J = 7.6 Hz, 1H), 5.01 (dd, J = |
| | 4.8, 13.2 Hz, 1H), 4.98-4.87 (m, 2H), 4.45 (s, 1H), 4.36 (d, J = 16.8 Hz, 1H), |
| | 4.20 (d, J = 16.8 Hz, 1H), 3.66 (s, 2H), 3.61 (s, 2H), 3.57 (d, J = 8.0 Hz, 2H), |
| | 3.30 (s, 3H), 3.24 (s, 2H), 2.94 (s, 1H), 2.88 (s, 3H), 2.86 (s, 2H), 2.78 (s, 3H), |
| | 2.71-2.59 (m, 2H), 2.57 (s, 1H), 2.47-2.31 (m, 4H), 1.96-1.85 (m, 4H), |
| | 1.84-1.79 (m, 1H), 1.75-1.66 (m, 2H), 1.51-1.41 (m, 2H), 1.37-1.28 (m, |
| | 1H), 0.99-0.88 (m, 2H) |
| 272 | ¹H NMR (400 MHz, DMSO-d₆) δ = 10.98 (s, 1H), 8.17 (s, 1H), 7.87-7.79 |
| | (m, 1H), 7.42 (s, 1H), 7.36-7.25 (m, 2H), 6.92 (d, J = 4.0 Hz, 2H), 6.81 (d, |
| | J = 8.0 Hz, 1H), 5.06 (dd, J = 5.2, 13.2 Hz, 1H), 4.94 (s, 2H), 4.45 (d, J = 17.2 |
| | Hz, 1H), 4.28 (d, J = 17.2 Hz, 1H), 3.69 (s, 2H), 3.63 (s, 2H), 3.29 (s, 2H), |
| | 3.25-3.19 (m, 1H), 2.95 (d, J = 10.8 Hz, 2H), 2.88 (s, 2H), 2.86 (s, 2H), 2.78 |
| | (s, 3H), 2.71-2.59 (m, 1H), 2.56 (s, 3H), 2.48-2.30 (m, 2H), 2.17-2.09 (m, |
| | 2H), 2.03-1.95 (m, 3H), 1.91 (d, J = 13.2 Hz, 2H), 1.83-1.77 (m, 1H), 1.72- |
| | 1.76 (m, J = 8.4 Hz, 5H), 1.56-1.45 (m, 3H), 1.09-0.93 (m, 2H). |
| 273 | ¹H NMR (400 MHz, DMSO-d₆) δ: 11.06 (s, 1H), 8.17 (s, 1H), 8.16 (s, 1H), |
| | 7.96 (s, 1H), 7.92 (s, 1H), 7.86 (dd, J = 8.4, 1.2 Hz, 1H), 7.46 (s, 1H), 7.34 (t, |
| | J = 8.0 Hz, 1H), 6.90 (d, J = 8.4 Hz, 1H), 6.87 (d, J = 1.2 Hz, 1H), 6.82 (d, J = |
| | 7.6 Hz, 1H), 6.63-6.58 (m, 1H), 5.26 (dd, J = 12.8, 5.2 Hz, 1H), 5.10 (s, 2H), |
| | 4.57 (td, J = 6.8, 14.0 Hz, 1H), 3.73-3.65 (m, 2H), 3.57-3.45 (m, 2H), 3.30 (s, |
| | 2H), 3.26-3.18 (m, 2H), 2.95-2.88 (m, 2H), 2.87-2.80 (m, 2H), 2.78 (s, 3H), |
| | 2.70-2.53 (m, 6H), 2.47-2.30 (m, 3H), 2.25-2.12 (m, 2H), 2.10-2.02 (m, 4H), |
| | 2.02-1.95 (m, 2H), 1.75 (d, J = 10.8 Hz, 3H), 1.70-1.51 (m, 5H), 1.43 (d, J = |
| | 6.8 Hz, 7H), 1.21 (q, J = 10.8 Hz, 2H), 0.93 (d, J = 12.0 Hz, 7H). |
| 274 | ¹H NMR (400 MHz, DMSO-d₆) δ: 11.08 (s, 1 H), 8.17 (s, 1 H), 8.02 (s, 1 H), |
| | 7.93 (s, 1 H), 7.84 (d, J = 8.0 Hz, 1 H), 7.47 (s, 1 H), 7.36 (t, J = 8.0 Hz, 1 H), |
| | 6.95-7.01 (m, 1 H), 6.84-6.92 (m, 3 H), 5.35 (dd, J = 12.4, 5.2 Hz, 1 H), 5.10 |
| | (s, 2 H), 3.68 (d, J = 7.6 Hz, 1 H), 3.63 (s, 3 H), 3.45-3.56 (m, 1 H), 3.35- |
| | 3.41 (m, 1 H), 3.34 (s, 1 H), 3.18-3.29 (m, 2 H), 3.03-3.12 (m, 2 H), 2.89- |
| | 2.93 (m, 3 H), 2.88 (s, 2 H), 2.85 (s, 1 H), 2.80 (s, 4 H), 2.71-2.78 (m, 2 H), |
| | 2.67-2.71 (m, 1 H), 2.65 (s, 1 H), 2.52-2.55 (m, 2 H), 2.36-2.41 (m, 1 H), |
| | 2.10-2.26 (m, 2 H), 1.94-2.05 (m, 6 H), 1.79 (d, J = 9.2 Hz, 3 H), 1.49-1.63 |
| | (m, 2 H), 1.36-1.48 (m, 1 H), 1.26 (s, 2 H), 1.13 (s, 6 H), 0.84-1.02 (m, 2 |
| | H). |
| 275 | ¹H NMR (400 MHz, DMSO-d₆) δ: 11.07 (s, 1 H), 8.18 (s, 1 H), 8.01 (s, 1 H), |
| | 7.93 (s, 1 H), 7.84 (d, J = 8.4 Hz, 1 H), 7.46 (s, 1 H), 7.36 (t, J = 8.0 Hz, 1 H), |
| | 6.96 (d, J = 8.0 Hz, 1 H), 6.86 (m, 3 H), 5.53-5.71 (m, 1 H), 5.29 (m, 1 H), |
| | 5.10 (s, 2 H), 3.45-3.57 (m, 1 H), 3.36 (s, 2 H), 3.18-3.29 (m, 2 H), 3.08 (d, |
| | J = 9.6 Hz, 2 H), 2.93-3.00 (m, 1 H), 2.89 (s, 2 H), 2.88 (s, 3 H), 2.84 (s, 1 H), |
| | 2.80 (s, 4 H), 2.65-2.75 (m, 3 H), 2.59 (s, 1 H), 2.38 (s, 1 H), 2.10-2.28 (m, |
| | 2 H), 1.88-2.08 (m, 7 H), 1.67-1.87 (m, 4 H), 1.48 (m, 9 H), 1.21-1.29 (m, |
| | 2 H), 1.03-1.19 (m, 6 H), 0.80-1.01 (m, 2 H). |
| 284 | ¹H NMR: (400 MHz, DMSO-d₆) δ = 11.06 (s, 1H), 8.17 (s, 1H), 7.84 (dd, J = |
| | 1.6, 8.2 Hz, 1H), 7.44 (s, 1H), 7.34 (t, J = 7.6 Hz, 1H), 7.00-6.93 (m, 3H), |
| | 6.90 (d, J = 7.6 Hz, 1H), 6.83 (t, J = 6.4 Hz, 2H), 5.70-5.57 (m, 1H), 5.29 |
| | (dd, J = 5.6, 12.4 Hz, 1H), 4.96 (s, 2H), 3.72 (s, 4H), 3.26-3.22 (m, 1H), 3.11 |
| | (d, J = 10.4 Hz, 2H), 2.88 (d, J = 8.0 Hz, 4H), 2.80 (s, 3H), 2.75-2.62 (m, |
| | 4H), 2.55 (s, 1H), 2.43-2.34 (m, 2H), 2.29-2.14 (m, 2H), 2.01-1.96 (m, |
| | 1H), 1.92-1.72 (m, 6H), 1.71-1.60 (m, 1H), 1.48 (dd, J = 6.8, 10.8 Hz, 6H), |
| | 1.34-1.19 (m, 2H) |
| 285 | ¹H NMR (400 MHz, DMSO-_{d6}) δ: 10.97 (s, 1H), 9.22 (s, 1H), 8.19 (d, J = 10.4 |
| | Hz, 2H), 8.08-7.99 (m, 1H), 7.88 (d, J = 6.4 Hz, 2H), 7.57-7.40 (m, 3H), 7.14 |
| | (t, J = 8.0 Hz, 1H), 7.03 (d, J = 7.6 Hz, 1H), 5.07 (dd, J = 5.2, 13.6 Hz, 1H), |
| | 5.00-4.96 (m, 3H), 4.56-4.22 (m, 2H), 3.68-3.53 (m, 4H), 3.52-3.36 (m, |
| | 4H), 3.01 (s, 3H), 2.84-2.55 (m, 6H), 2.44-2.31 (m, 4H), 2.18-1.90 (m, 8H), |
| | 1.82-1.70 (m, 3H), 1.69-1.56 (m, 4H), 1.52-1.40 (m, 1H), 1.30-1.14 (m, |
| | 2H), 0.92 (d, J = 12.4 Hz, 6H). |
| 286 | ¹H NMR: (400 MHz, DMSO-d₆) δ: 10.97 (s, 1H), 8.19 (s, 1H), 7.95 (s, 1H), |
| | 7.93-7.84 (m, 2H), 7.46 (d, J = 8.4 Hz, 1H), 7.40 (s, 1H), 7.35 (t, J = 8.0 Hz, |
| | 1H), 7.16 (t, J = 8.0 Hz, 1H), 6.76 (d, J = 8.0 Hz, 1H), 5.18-5.02 (m, 3H), 4.97 |
| | (d, J = 6.0 Hz, 2H), 4.89 (d, J = 6.0 Hz, 2H), 4.52-4.26 (m, 2H), 3.76-3.56 |
| | (m, 3H), 3.51 (s, 2H), 3.42-3.35 (m, 3H), 3.29 (d, J = 3.6 Hz, 1H), 2.98-2.86 |
| | (m, 6H), 2.68-2.53 (m, 3H), 2.48-2.31 (m, 4H), 2.13-1.83 (m, 12H), 1.79- |
| | 1.69 (m, 3H), 1.65-1.52 (m, 5H), 1.48-1.33 (m, 2H), 1.12 (d, J = 12.0 Hz, |
| | 2H), 0.90 (d, J = 11.6 Hz, 6H), 0.83 (d, J = 12.0 Hz, 3H) |
| 287 | ¹H NMR: (400 MHz, DMSO-d₆) δ: 10.93-10.83 (d, 1H), 8.18 (s, 1H), 7.98 (s, |
| | 1H), 7.92 (s, 1H), 7.84 (m, 1H), 7.72-7.63 (m, 1H), 7.48-7.45 (m, 1H), |
| | 7.38-7.31 (m, 1H), 6.88-6.81 (m, 2H), 5.11 (s, 2H), 4.74-4.67 (m, 1H), 4.50- |
| | 4.40 (m, 3H), 3.69-3.65 (m, 2H), 3.36 (m, 2H), 3.25-3.23 (m, 2H), 2.99-2.93 |
| | (m, 2H), 2.89 (s, 2H), 2.88 (s, 2H), 2.81 (m, 2H), 2.79 (s, 3H), 2.63-2.56 (m, |
| | 2H), 2.46-2.43 (m, 4H), 2.05 (m, 3H), 2.01-1.94 (m, 4H), 1.84-1.74 (m, 4H), |
| | 1.72-1.64 (m, 2H), 1.42-1.38 (m, 2H), 1.37 (d, J = 6.8 Hz, 2H), 1.14-1.02 |
| | (m, 5H), 0.95 (s, 6H) |
| 288 | ¹H NMR: (400 MHz, DMSO-d₆) δ: 11.06 (s, 1H), 8.17 (s, 1H), 8.15 (s, 1H), |
| | 7.96 (s, 1H), 7.92 (s, 1H), 7.88-7.84 (m, 1H), 7.46 (s, 1H), 7.34 (t, J = 8.0 Hz, |
| | 1H), 6.99-6.93 (m, 1H), 6.88 (d, J = 8.4 Hz, 1H), 6.82 (d, J = 7.6 Hz, 2H), 5.66- |
| | 5.58 (m, 1H), 5.32-5.25 (m, 1H), 5.11 (s, 2H), 3.67 (q, J = 14.4 Hz, 2H), 3.30 |
| | (s, 3H), 3.26-3.18 (m, 3H), 3.09-3.04 (m, 2H), 2.93-2.88 (m, 3H), 2.88- |
| | 2.85 (m, 2H), 2.79 (s, 3H), 2.72-2.56 (m, 6H), 2.45-2.35 (m, 2H), 2.11-2.02 |
| | (m, 4H), 2.01-1.94 (m, 2H), 1.84-1.68 (m, 4H), 1.66-1.56 (m, 4H), 1.50- |
| | 1.40 (m, 8H), 1.27-1.15 (m, 2H), 0.93 (d, J = 11.6 Hz, 6H) |
| 289 | ¹H NMR: (400 MHz, DMSO-d₆) δ: 11.10 (s, 1H), 8.25-8.35 (m, 1H), 8.21 (s, |
| | 1H), 8.01 (s, 2H), 7.83-7.93 (m, 1H), 7.50 (s, 1H), 7.34-7.43 (m, 1H), 6.96-7.03 |
| | (m, 1H), 6.83-6.94 (m, 3H), 5.54-5.80 (m, 1H), 5.24-5.43 (m, 1H), 5.14 (s, 2H), |
| | 3.87 (s, 2H), 3.34 (br s, 2H), 3.28 (br s, 2H), 3.10 (br d, J = 11.2 Hz, 2H), 2.87- |
| | 2.95 (m, 5H), 2.83 (s, 3H), 2.70-2.74 (m, 2H), 2.66-2.70 (m, 5H), 2.63 (br d, J = |
| | 6.0 Hz, 2H), 2.58 (s, 3H), 2.28 (br d, J = 6.8 Hz, 2H), 1.91-2.09 (m, 5H), 1.73- |
| | 1.87 (m, 5H), 1.45-1.61 (m, 8H), 1.36-1.44 (m, 1H), 1.12-1.24 (m, 2H), 0.85- |
| | 0.97 ppm (m, 2H) |
| 290 | ¹H NMR: (400 MHz, DMSO-d₆) δ: 11.06-10.89 (m, 1H), 8.16 (s, 1H), 7.94 (br |
| | d, J = 14.4 Hz, 2H), 7.85 (br d, J = 8.8 Hz, 1H), 7.49-7.42 (m, 2H), 7.37-7.31 |
| | (m, 1H), 7.20-7.11 (m, 1H), 6.86-6.78 (m, 1H), 5.16-5.00 (m, 3H), 4.52- |
| | 4.42 (m, 1H), 4.36-4.25 (m, 1H), 3.64 (s, 1H), 3.72-3.59 (m, 2H), 3.58-3.51 |
| | (m, 2H), 3.26-3.18 (m, 3H), 2.96-2.85 (m, 7H), 2.78 (s, 3H), 2.69-2.58 (m, |
| | 4H), 2.56 (br s, 1H), 2.45-2.38 (m, 2H), 2.27-2.20 (m, 1H), 2.12-2.04 (m, |
| | 1H), 2.00-1.94 (m, 1H), 1.92-1.85 (m, 2H), 1.83-1.74 (m, 3H), 1.74-1.69 |
| | (m, 1H), 1.61 (br t, J = 10.4 Hz, 3H), 1.57-1.49 (m, 2H), 1.46-1.39 (m, 1H), |
| | 1.24 (br d, J = 8.4 Hz, 2H), 1.03 (s, 3H), 0.96-0.90 (m, 1H), 0.88 (s, 3H) |
| 291 | ¹H NMR: (400 MHz, DMSO-d₆) δ: 11.04-10.91 (m, 1H), 8.16 (s, 1H), 7.96 (s, |
| | 1H), 7.91 (s, 1H), 7.85 (br d, J = 8.4 Hz, 1H), 7.44 (s, 1H), 7.46 (s, 1H), 7.33 (t, |
| | J = 8.0 Hz, 1H), 7.18-7.11 (m, 1H), 6.82 (d, J = 8.2 Hz, 1H), 5.14-5.03 (m, |
| | 3H), 4.47 (d, J = 17.2 Hz, 1H), 4.30 (d, J = 16.8 Hz, 1H), 3.75-3.68 (m, 1H), |
| | 3.67-3.61 (m, 1H), 3.60-3.50 (m, 2H), 3.36-3.32 (m, 2H), 3.29 (br s, 2H), |
| | 3.25-3.20 (m, 1H), 2.95-2.89 (m, 3H), 2.89-2.85 (m, 4H), 2.78 (s, 3H), 2.71- |
| | 2.58 (m, 3H), 2.58-2.53 (m, 1H), 2.48-2.35 (m, 3H), 2.25-2.16 (m, 1H), |
| | 2.03-1.94 (m, 2H), 1.91-1.78 (m, 4H), 1.73-1.45 (m, 8H), 1.20-1.11 (m, |
| | 2H), 0.98 (s, 3H), 0.87 (s, 3H) |
| 292 | ¹H NMR: (400 MHz, DMSO-d₆) δ = 10.92 (s, 1H), 9.22 (br s, 1H), 8.18 (s, 1H), |
| | 8.05-7.99 (m, 1H), 7.90 (br d, J = 6.0 Hz, 2H), 7.53-7.44 (m, 2H), 7.06 (br d, |
| | J = 7.6 Hz, 1H), 6.93-6.86 (m, 1H), 5.06-4.98 (m, 1H), 4.43-4.35 (m, 1H), |
| | 4.27-4.18 (m, 1H), 3.67-3.54 (m, 2H), 3.38-3.37 (m, 2H), 3.27-3.24 (m, |
| | 2H), 3.01-2.95 (m, 2H), 2.92 (br d, J = 8.0 Hz, 4H), 2.88 (br s, 3H), 2.60 (br |
| | d, J = 2.0 Hz, 2H), 2.52 (br s, 3H), 2.47-2.43 (m, 2H), 2.39 (br s, 3H), 2.32- |
| | 2.06 (m, 4H), 2.05-1.94 (m, 4H), 1.94-1.88 (m, 3H), 1.82-1.74 (m, 2H), |
| | 1.59-1.51 (m, 2H), 1.46-1.38 (m, 1H), 1.26-1.17 (m, 2H), 1.13 (br s, 7H), |
| | 0.92-0.83 (m, 2H) |
| 293 | ¹H NMR: (400 MHz, DMSO-d₆) δ: 11.09 (s, 1H), 8.18 (s, 1H), 7.99 (s, 1H), |
| | 7.95 (s, 1H), 7.86 (dd, J = 1.2, 8.0 Hz, 1H), 7.46 (s, 1H), 7.36 (t, J = 8.0 Hz, |
| | 1H), 7.01-6.93 (m, 1H), 6.91-6.80 (m, 3H), 5.62 (qd, J = 6.4, 12.8 Hz, 1H), |
| | 5.30 (dd, J = 5.2, 12.4 Hz, 1H), 5.12 (s, 2H), 3.72 (s, 2H), 3.56-3.46 (m, 2H), |
| | 3.39 (dd, J = 5.6, 6.8 Hz, 4H), 3.28-3.22 (m, 2H), 3.11-2.97 (m, 4H), 2.90 (s, |
| | 2H), 2.89-2.84 (m, 4H), 2.80 (s, 3H), 2.76-2.56 (m, 4H), 2.12-1.92 (m, 10H), |
| | 1.85-1.75 (m, 1H), 1.53-1.43 (m, 9H), 1.34-1.20 (m, 4H). |
| 294 | ¹H NMR: (400 MHz, DMSO-d₆) δ: 10.89 (d, J = 12.8 Hz, 1 H), 8.14 (s, 1 H), |
| | 7.98-8.10 (m, 1 H), 7.93 (s, 1 H), 7.78 (d, J = 8.8 Hz, 1 H), 7.70 (dd, J = 8.8, 2.8 |
| | Hz, 1 H), 7.26-7.35 (m, 2 H), 6.89 (dd, J = 8.8, 3.2 Hz, 1 H), 6.63-6.75 (m, 1 |
| | H), 5.07 (s, 2 H), 4.70 (dd, J = 12.4, 4.8 Hz, 1 H), 4.37-4.55 (m, 1 H), 4.11- |
| | 4.21 (m, 2 H), 4.05-4.11 (m, 1 H), 3.72 (s, 1 H), 3.37-3.44 (m, 4 H), 3.31 (s, |
| | 2 H), 3.17 (s, 3 H), 3.12 (s, 2 H), 2.97-3.02 (m, 2 H), 2.78-2.91 (m, 2 H), 2.70 |
| | (s, 3 H), 2.55-2.67 (m, 2 H), 2.54 (d, J = 1.2 Hz, 2 H), 2.37 (d, J = 2.8 Hz, 1 H), |
| | 2.17-2.28 (m, 1 H), 2.07-2.17 (m, 3 H), 1.92-2.03 (m, 4 H), 1.83-1.89 (m, |
| | 2 H), 1.72-1.81 (m, 2 H), 1.47-1.62 (m, 1 H), 1.39 (dd, J = 14.4, 6.75 Hz, 6 H), |
| | 1.19-1.29 (m, 3 H), 1.13 (s, 4 H), 0.97-1.09 (m, 1 H), 0.83-0.93 (m, 1 H) |
| 295 | ¹H NMR: (400 MHz, DMSO-d₆) δ: 11.06 (s, 1H), 8.30 (s, 2H), 8.19 (s, 1H), |
| | 8.10-7.92 (m, 1H), 7.91-7.77 (m, 2H), 7.76-7.58 (m, 1H), 7.58-7.46 (m, |
| | 2H), 7.42-7.29 (m, 1H), 6.96 (d, J = 7.2 Hz, 2H), 6.86 (d, J = 8.0 Hz, 1H), 6.65 |
| | (d, J = 8.4 Hz, 1H), 5.28 (dd, J = 5.2, 12.4 Hz, 1H), 5.19 (s, 2H), 4.66-4.56 (m, |
| | 1H), 3.93-3.53 (m, 2H), 3.40 (s, 3H), 3.29-3.22 (m, 2H), 3.21-2.97 (m, 4H), |
| | 2.90 (d, J = 8.0 Hz, 4H), 2.82 (s, 3H), 2.70-2.57 (m, 4H), 2.56-2.53 (m, 1H), |
| | 2.04-1.93 (m, 1H), 1.46 (s, 3H), 1.45 (s, 3H). |
| 296 | ¹H NMR: (400 MHz, DMSO-d₆) δ: 11.09 (s, 1H), 8.28 (d, J = 1.2 Hz, 2H), 8.19 |
| | (s, 1H), 7.92-7.84 (m, 3H), 7.56-7.49 (m, 3H), 7.37 (t, J = 8.0 Hz, 1H), 7.02- |
| | 6.92 (m, 2H), 6.85 (t, J = 6.8 Hz, 2H), 5.75-5.62 (m, 1H), 5.30 (dd, J = 5.6, |
| | 12.4 Hz, 1H), 5.18 (s, 2H), 3.69-3.59 (m, 2H), 3.32 (s, 2H), 3.30-3.20 (m, |
| | 2H), 3.02 (d, J = 10.4 Hz, 2H), 2.93 (s, 1H), 2.91 (s, 3H), 2.89-2.83 (m, 4H), |
| | 2.80 (s, 3H), 2.70-2.58 (m, 2H), 2.34-2.26 (m, 2H), 2.04-1.93 (m, 1H), 1.48 |
| | (dd, J = 6.8, 11.2 Hz, 6H). |
| 297 | ¹H NMR: (400 MHz, DMSO-d₆) δ = 11.00 (s, 1H), 9.24 (s, 1H), 8.20 (s, 1H), |
| | 8.15 (s, 1H), 8.02 (br d, J = 7.4 Hz, 1H), 7.89 (br d, J = 9.6 Hz, 2H), 7.54 (s, |
| | 1H), 7.41-7.49 (m, 2H), 7.09 (t, J = 7.6 Hz, 1H), 7.00 (d, J = 7.2 Hz, 1H), 5.08 |
| | (dd, J = 13.2, 4.8 Hz, 1H), 4.92-5.00 (m, 4H), 4.47 (d, J = 17.6 Hz, 1H), 4.30 |
| | (d, J = 17.2 Hz, 1H), 3.55-3.66 (m, 4H), 3.44-3.54 (m, 2H), 3.00 (s, 3H), 2.70- |
| | 2.90 (m, 6H), 2.55-2.64 (m, 3H), 2.35-2.45 (m, 2H), 2.17-2.27 (m, 1H), 2.06- |
| | 2.16 (m, 2H), 1.95-2.01 (m, 1H), 1.74-1.87 (m, 6H), 1.56-1.70 (m, 4H), 1.43- |
| | 1.54 (m, 3H), 1.30-1.40 (m, 1H), 0.89-1.12 ppm (m, 2H) |
| 298 | ¹H NMR (400 MHz, DMSO-d₆) δ: 10.98 (s, 1 H), 9.24 (s, 1 H), 8.21 (s, 1 H), |
| | 8.15 (s, 1 H), 8.04 (m, 1 H), 7.90 (d, J = 8.4 Hz, 2 H), 7.54 (s, 1 H), 7.47-7.52 |
| | (m, 2 H), 7.03-7.18 (m, 2 H), 5.08 (m, 1 H), 4.95-5.02 (m, 4 H), 4.49 (d, |
| | J = 16.8 Hz, 1 H), 4.32 (d, J = 16.8 Hz, 1 H), 3.66 (d, J = 11.2 Hz, 3 H), 3.59 (s, 3 |
| | H), 3.11 (s, 4 H), 3.02 (s, 3 H), 2.86-3.01 (m, 4 H), 2.81 (d, J = 7.6 Hz, 1 H), |
| | 2.72 (d, J = 10.0 Hz, 1 H), 2.59 (d, J = 16.8 Hz, 1 H), 2.48 (s, 3 H), 2.38-2.45 (m, |
| | 2 H), 2.14 (d, J = 6.8 Hz, 4 H), 1.95-2.01 (m, 1 H), 1.86 (s, 2 H), 1.61-1.73 (m, |
| | 4 H), 1.47-1.58 (m, 2 H), 0.95-1.17 (m, 3 H) |
| 299 | ¹H NMR (400 MHz, DMSO-d₆) δ: 10.97 (s, 1H), 9.22 (s, 1H), 8.20 (s, 1H), 8.16 |
| | (s, 3H), 8.04 (dd, J = 1.6, 8.4 Hz, 1H), 7.89 (d, J = 8.4 Hz, 2H), 7.54 (s, 1H), |
| | 7.52 (s, 2H), 7.16 (t, J = 8.0 Hz, 1H), 7.05 (d, J = 8.0 Hz, 1H), 5.08 (dd, J = 5.2, |
| | 13.2 Hz, 1H), 5.02-4.95 (m, 4H), 4.49 (d, J = 17.2 Hz, 1H), 4.32 (d, J = 16.8 |
| | Hz, 1H), 3.65 (s, 2H), 3.59 (s, 2H), 3.14 (s, 4H), 3.02 (s, 3H), 3.00-2.92 (m, |
| | 3H), 2.86 (d, J = 10.4 Hz, 3H), 2.63-2.56 (m, 1H), 2.47 (s, 2H), 2.42 (dd, J = |
| | 4.4, 13.2 Hz, 1H), 2.35-2.30 (m, 2H), 2.20 (d, J = 7.2 Hz, 2H), 2.17-1.88 (m, |
| | 6H), 1.75-1.65 (m, 4H), 1.63-1.53 (m, 2H), 1.23-1.11 (m, 4H) |
| 300 | ¹H NMR (400 MHz, DMSO-d₆) δ = 11.07 (s, 1H), 8.17 (s, 1H), 8.01 (s, 1H), |
| | 7.92 (s, 1H), 7.88-7.77 (m, 1H), 7.47 (s, 1H), 7.40-7.26 (m, 2H), 6.84 (d, J = |
| | 8.0 Hz, 1H), 6.45 (d, J = 8.8 Hz, 1H), 5.42-5.23 (m, 1H), 5.09 (s, 2H), 3.94- |
| | 3.87 (m, 2H), 3.73-3.59 (m, 2H), 3.28 (s, 4H), 3.24 (d, J = 9.6 Hz, 1H), 3.04 |
| | (t, J = 10.4 Hz, 2H), 2.93-2.85 (m, 5H), 2.79 (s, 3H), 2.70-2.62 (m, 2H), 2.61- |
| | 2.55 (m, 2H), 2.35 (dd, J = 2.8, 12.0 Hz, 3H), 2.29-2.11 (m, 3H), 2.02 (d, |
| | J = 7.2 Hz, 4H), 1.96-1.90 (m, 2H), 1.89-1.83 (m, 2H), 1.82-1.76 (m, 2H), |
| | 1.47-1.38 (m, 3H), 1.16 (s, 2H), 1.12 (s, 6H), 0.94-0.84 (m, 2H) |
| 301 | ¹H NMR: (400 MHz, DMSO-d₆) δ: 11.08 (s, 1 H), 8.17 (s, 1 H), 8.02 (s, 1 H), |
| | 7.93 (s, 1 H), 7.84 (d, J = 8.0 Hz, 1 H), 7.47 (s, 1 H), 7.36 (t, J = 8.0 Hz, 1 H), |
| | 6.95-7.01 (m, 1 H), 6.84-6.92 (m, 3 H), 5.35 (dd, J = 12.4, 5.2 Hz, 1 H), 5.10 (s, 2 |
| | H), 3.68 (d, J = 7.6 Hz, 1 H), 3.63 (s, 3 H), 3.45-3.56 (m, 1 H), 3.35-3.41 (m, |
| | 1 H), 3.34 (s, 1 H), 3.18-3.29 (m, 2 H), 3.03-3.12 (m, 2 H), 2.89-2.93 (m, 3 |
| | H), 2.88 (s, 2 H), 2.85 (s, 1 H), 2.80 (s, 4 H), 2.71-2.78 (m, 2 H), 2.67-2.71 |
| | (m, 1 H), 2.65 (s, 1 H), 2.52-2.55 (m, 2 H), 2.36-2.41 (m, 1 H), 2.10-2.26 |
| | (m, 2 H), 1.94-2.05 (m, 6 H), 1.79 (d, J = 9.2 Hz, 3 H), 1.49-1.63 (m, 2 H), |
| | 1.36-1.48 (m, 1 H), 1.26 (s, 2 H), 1.13 (s, 6 H), 0.84-1.02 (m, 2 H) |
| 302 | ¹H NMR (400 MHz, DMSO-d₆) δ: 11.07 (s, 1 H), 8.18 (s, 1 H), 8.01 (s, 1 H), |
| | 7.93 (s, 1 H), 7.84 (d, J = 8.4 Hz, 1 H), 7.46 (s, 1 H), 7.36 (t, J = 8.0 Hz, 1 H), 6.96 |
| | (d, J = 8.0 Hz, 1 H), 6.86 (m, 3 H), 5.53-5.71 (m, 1 H), 5.29 (m, 1 H), 5.10 (s, |
| | 2 H), 3.45-3.57 (m, 1 H), 3.36 (s, 2 H), 3.18-3.29 (m, 2 H), 3.08 (d, J = 9.6 Hz, |
| | 2 H), 2.93-3.00 (m, 1 H), 2.89 (s, 2 H), 2.88 (s, 3 H), 2.84 (s, 1 H), 2.80 (s, 4 |
| | H), 2.65-2.75 (m, 3 H), 2.59 (s, 1 H), 2.38 (s, 1 H), 2.10-2.28 (m, 2 H), 1.88- |
| | 2.08 (m, 7 H), 1.67-1.87 (m, 4 H), 1.48 (m, 9 H), 1.21-1.29 (m, 2 H), 1.03- |
| | 1.19 (m, 6 H), 0.80-1.01 (m, 2 H) |
| 303 | ¹H NMR: (400 MHz, DMSO-d₆) δ = 10.88 (d, J = 12.4 Hz, 1H), 8.18 (s, 1H), |
| | 8.01 (s, 1H), 7.93 (s, 1H), 7.84 (dd, J = 1.6, 8.0 Hz, 1H), 7.68 (dd, J = 2.8, 8.8 |
| | Hz, 1H), 7.50-7.41 (m, 1H), 7.35 (t, J = 8.0 Hz, 1H), 6.90-6.82 (m, 2H), 5.14- |
| | 5.06 (m, 2H), 4.77-4.64 (m, 1H), 4.53-4.39 (m, 3H), 3.31 (s, 3H), 3.25 (s, |
| | 1H), 3.00-2.90 (m, 2H), 2.90-2.85 (m, 4H), 2.82 (d, J = 3.2 Hz, 2H), 2.79 (s, |
| | 3H), 2.78-2.75 (m, 1H), 2.62-2.55 (m, 1H), 2.54 (d, J = 4.0 Hz, 2H), 2.48- |
| | 2.45 (m, 2H), 2.36 (d, J = 2.4 Hz, 2H), 2.11 (d, J = 5.6 Hz, 2H), 2.07 (d, J = 6.8 |
| | Hz, 2H), 2.01-1.96 (m, 1H), 1.91-1.72 (m, 6H), 1.70-1.64 (m, 2H), 1.49- |
| | 1.43 (m, 1H), 1.39 (dd, J = 6.8, 14.8 Hz, 3H), 1.12 (s, 8H), 1.09-1.02 (m, 3H) |
| 304 | ¹H NMR: (400 MHz, DMSO-d₆) δ = 10.91-10.84 (m, 1H), 8.16 (s, 1H), 7.94 |
| | (s, 1H), 7.88 (s, 1H), 7.84 (dd, J = 1.6, 8.0 Hz, 1H), 7.69 (dd, J = 3.2, 8.8 Hz, |
| | 1H), 7.44 (s, 1H), 7.34 (t, J = 8.0 Hz, 1H), 6.88 (dd, J = 3.2, 8.8 Hz, 1H), 6.83 |
| | (d, J = 7.6 Hz, 1H), 5.08 (s, 2H), 4.69 (br dd, J = 5.2, 12.8 Hz, 1H), 4.52-4.38 |
| | (m, 1H), 4.15-4.03 (m, 2H), 3.96 (br s, 2H), 3.75-3.67 (m, 1H), 3.38-3.34 |
| | (m, 2H), 3.29 (br s, 2H), 3.26-3.19 (m, 2H), 2.88 (s, 2H), 2.86 (s, 2H), 2.78 (s, |
| | 4H), 2.73-2.66 (m, 2H), 2.62-2.57 (m, 1H), 2.56-2.52 (m, 1H), 2.40-2.36 |
| | (m, 2H), 2.30-2.14 (m, 2H), 2.13-2.07 (m, 2H), 2.03-1.96 (m, 1H), 1.93 (br |
| | d, J = 11.6 Hz, 2H), 1.85 (br dd, J = 3.2, 8.8 Hz, 2H), 1.78 (br d, J = 12.0 Hz, |
| | 2H), 1.45-1.34 (m, 6H), 1.17-1.08 (m, 2H), 0.95-0.82 (m, 2H), 0.64 (s, 2H), |
| | 0.46 (s, 2H) |
| 305 | ¹H NMR: (400 MHz, DMSO-d₆) δ: 10.90 (d, J = 12.4 Hz, 1H), 8.17 (s, 1H), |
| | 7.94 (s, 1H), 7.88 (s, 1H), 7.86-7.82 (m, 1H), 7.69 (dd, J = 3.2, 8.8 Hz, 1H), |
| | 7.44 (s, 1H), 7.34 (t, J = 7.8 Hz, 1H), 6.88 (dd, J = 3.6, 8.8 Hz, 1H), 6.82 (d, |
| | J = 8.0 Hz, 1H), 5.08 (s, 2H), 4.74-4.65 (m, 1H), 4.52-4.38 (m, 1H), 4.14-4.04 |
| | (m, 2H), 4.01-3.88 (m, 2H), 3.75-3.66 (m, 1H), 3.38-3.36 (m, 1H), 3.29 (s, |
| | 3H), 3.27-3.19 (m, 2H), 2.90-2.84(m, 4H), 2.80-2.74 (m, 4H), 2.71-2.65 |
| | (m, 2H), 2.60-2.56 (m, 1H), 2.41-2.33 (m, 3H), 2.29-2.15 (m, 2H), 2.11- |
| | 2.07 (m, 2H), 2.02-1.97 (m, 1H), 1.95-1.90 (m, 2H), 1.87-1.81 (m, 2H), 1.80- |
| | 1.75 (m, 2H), 1.44-1.34 (m, 6H), 1.19-1.08 (m, 2H), 0.93-0.83 (m, 2H), |
| | 0.64 (s, 2H), 0.46 (s, 2H) |
| 306 | ¹H NMR: (400 MHz, DMSO-d₆) δ = 10.93-10.84 (m, 1H), 8.17 (s, 1H), 8.00 |
| | (s, 1H), 7.91 (s, 1H), 7.83 (dd, J = 1.2, 8.0 Hz, 1H), 7.72-7.66 (m, 1H), 7.46 |
| | (s, 1H), 7.34 (t, J = 8.0 Hz, 1H), 6.88 (dd, J = 3.6, 8.8 Hz, 1H), 6.82 (d, J = 7.6 |
| | Hz, 1H), 5.08 (s, 2H), 4.69 (br dd, J = 5.2, 12.4 Hz, 1H), 4.53-4.37 (m, 1H), |
| | 4.09 (br dd, J = 14.0, 17.6 Hz, 2H), 3.83-3.41 (m, 3H), 3.36 (br dd, J = 1.2, 2.4 |
| | Hz, 2H), 3.30 (s, 2H), 3.28-3.18 (m, 2H), 2.91-2.88 (m, 2H), 2.87 (br s, 2H), |
| | 2.78 (s, 4H), 2.62-2.57 (m, 1H), 2.57-2.51 (m, 2H), 2.36 (br s, 2H), 2.31- |
| | 2.09 (m, 3H), 2.05-1.96 (m, 3H), 1.93 (br d, J = 11.2 Hz, 2H), 1.88-1.82 (m, |
| | 2H), 1.78 (br d, J = 11.6 Hz, 2H), 1.38 (br dd, J = 6.8, 14.4 Hz, 6H), 1.20-1.08 |
| | (m, 8H), 0.95-0.80 (m, 2H) |
| 307 | ¹H NMR: (400 MHz, DMSO-d₆) δ = 10.89 (br d, J = 12.4 Hz, 1H), 8.20-8.15 |
| | (m, 1H), 8.02-7.98 (m, 1H), 7.95-7.90 (m, 1H), 7.83 (br d, J = 9.2 Hz, 1H), |
| | 7.69 (dd, J = 3.2, 8.8 Hz, 1H), 7.46 (s, 1H), 7.34 (t, J = 8.0 Hz, 1H), 6.89 (br dd, |
| | J = 3.2, 8.8 Hz, 1H), 6.82 (br d, J = 8.0 Hz, 1H), 5.08 (s, 2H), 4.69 (br dd, J = |
| | 5.2, 12.8 Hz, 1H), 4.52-4.38 (m, 1H), 4.13-4.00 (m, 2H), 3.74-3.60 (m, 3H), |
| | 3.42-3.36 (m, 2H), 3.30 (br s, 2H), 3.26-3.19 (m, 2H), 2.88 (s, 2H), 2.86 (br |
| | s, 2H), 2.82-2.74 (m, 4H), 2.61-2.57 (m, 1H), 2.53 (br d, J = 1.2 Hz, 2H), |
| | 2.35 (br s, 2H), 2.29-2.10 (m, 3H), 2.05 (br d, J = 6.4 Hz, 2H), 2.02-1.92 (m, |
| | 2H), 1.84 (br d, J = 7.2 Hz, 2H), 1.74-1.64 (m, 2H), 1.50 (br s, 3H), 1.38 (br |
| | dd, J = 6.8, 14.2 Hz, 6H), 1.29-1.23 (m, 2H), 1.11 (s, 6H) |
| 308 | ¹H NMR: (400 MHz, DMSO-d₆) δ: 10.89 (d, J = 12.8 Hz, 1H), 8.17 (s, 1H), |
| | 8.00 (s, 1H), 7.92 (s, 1H), 7.84 (dd, J = 1.6, 8.0 Hz, 1H), 7.70 (dd, J = 3.2, 8.8 |
| | Hz, 1H), 7.46 (s, 1H), 7.35 (t, J = 8.0 Hz, 1H), 6.89 (dd, J = 3.6, 9.2 Hz, 1H), |
| | 6.83 (d, J = 7.6 Hz, 1H), 5.09 (s, 2H), 4.74-4.65 (m, 1H), 4.52-4.39 (m, 1H), |
| | 4.14-4.04 (m, 2H), 3.76-3.67 (m, 2H), 3.33 (s, 1H), 3.30 (s, 3H), 3.27-3.19 |
| | (m, 2H), 2.91-2.85 (m, 4H), 2.84-2.77 (m, 4H), 2.66-2.52 (m, 4H), 2.39- |
| | 2.33 (m, 2H), 2.23-2.12 (m, 2H), 2.05-1.90 (m, 6H), 1.88-1.82 (m, 2H), |
| | 1.81-1.75 (m, 2H), 1.44-1.35 (m, 6H), 1.22-1.14 (m, 2H), 1.12 (s, 6H), 0.94- |
| | 0.82 (m, 2H) |
| 309 | ¹H NMR: (400 MHz, DMSO-d₆) δ: 10.89 (d, J = 12.4 Hz, 1H), 8.17 (s, 1H), |
| | 8.00 (s, 1H), 7.92 (s, 1H), 7.84 (d, J = 7.6 Hz, 1H), 7.69 (dd, J = 3.2, 8.8 Hz, |
| | 1H), 7.46 (s, 1H), 7.34 (t, J = 7.6 Hz, 1H), 6.89 (dd, J = 3.6, 8.8 Hz, 1H), 6.82 |
| | (d, J = 6.8 Hz, 1H), 5.08 (s, 2H), 4.73-4.66 (m, 1H), 4.52-4.39 (m, 1H), |
| | 4.12-4.03 (m, 2H), 3.73-3.61 (m, 3H), 3.35-3.35 (m, 1H), 3.30 (s, 3H), 3.25- |
| | 3.23 (m, 2H), 2.89-2.85 (m, 4H), 2.78 (s, 4H), 2.61-2.55 (m, 2H), 2.37-2.34 |
| | (m, 2H), 2.05 (d, J = 6.4 Hz, 2H), 1.88-1.82 (m, 2H), 1.73-1.66 (m, 2H), |
| | 1.58-1.50 (m, 2H), 1.48-1.34 (m, 10H), 1.33-1.22 (m, 4H), 1.14-1.10 (m, 7H) |
| 310 | ¹H NMR: (400 MHz, DMSO-d₆) δ = 10.87 (s, 1H), 9.45-8.83 (m, 1H), 8.18 |
| | (s, 2H), 7.84 (d, J = 8.0 Hz, 2H), 7.71 (d, J = 8.0 Hz, 1H), 7.45 (s, 1H), 7.36 (t, |
| | J = 7.6 Hz, 1H), 6.98-6.73 (m, 2H), 5.21-5.01 (m, 2H), 4.76-4.67 (m, 1H), |
| | 4.55-4.40 (m, 3H), 4.23-3.99 (m, 1H), 3.97-3.73 (m, 1H), 3.56-3.44 (m, |
| | 2H), 3.31 (s, 4H), 3.27-3.19 (m, 2H), 3.06-2.92 (m, 6H), 2.88 (d, J = 7.6 Hz, |
| | 4H), 2.81 (s, 3H), 2.76 (d, J = 13.6 Hz, 2H), 2.60 (s, 2H), 2.55 (s, 1H), 2.49- |
| | 2.43 (m, 2H), 2.42-2.32 (m, 2H), 2.28-2.21 (m, 1H), 2.19-2.07 (m, 1H), |
| | 2.02-1.95 (m, 1H), 1.88-1.76 (m, 3H), 1.69-1.48 (m, 1H), 1.44-1.37 (m, |
| | 3H), 1.34-1.04 (m, 3H), 1.02-0.86 (m, 1H), 0.72-0.28 (m, 3H) |
| 311 | ¹H NMR: (400 MHz, DMSO-d₆) δ: 10.88 (d, J = 12.4 Hz, 1H), 8.17 (s, 1H), |
| | 7.98 (s, 1H), 7.94 (s, 1H), 7.83 (d, J = 8.4 Hz, 1H), 7.69 (dd, J = 2.8, 8.4 Hz, |
| | 1H), 7.47 (s, 1H), 7.34 (t, J = 8.0 Hz, 1H), 6.90-6.85 (m, 1H), 6.82 (d, J = 7.6 |
| | Hz, 1H), 5.10 (s, 2H), 4.73-4.65 (m, 1H), 4.51-4.39 (m, 1H), 4.13-4.03 (m, |
| | 2H), 3.73-3.68 (m, 1H), 3.66-3.61 (m, 2H), 3.31-3.28 (m, 4H), 3.27-3.19 |
| | (m, 2H), 2.90-2.85 (m, 4H), 2.78 (s, 4H), 2.62-2.56 (m, 2H), 2.14-2.03 (m, |
| | 4H), 1.99-1.89 (m, 4H), 1.87-1.75 (m, 4H), 1.44-1.33 (m, 6H), 1.32-1.21 |
| | (m, 2H), 1.19-1.04 (m, 3H), 0.93 (s, 6H), 0.87-0.79 (m, 2H) |
| 312 | ¹H NMR: (400 MHz, DMSO-d₆) δ: 1.06 (br s, 1H), 8.17 (s, 1H), 8.00 (s, 1H), |
| | 7.92 (s, 1H), 7.83 (d, J = 9.2 Hz, 1H), 7.46 (s, 1H), 7.35 (t, J = 8.0 Hz, 1H), |
| | 7.00-6.93 (m, 1H), 6.89-6.77 (m, 3H), 5.27 (dd, J = 4.8, 12.0 Hz, 1H), 5.09 (s, |
| | 2H), 3.27-3.21 (m, 2H), 3.19-3.08 (m, 3H), 2.88 (d, J = 7.6 Hz, 5H), 2.79 (s, |
| | 5H), 2.70-2.60 (m, 3H), 2.40-2.30 (m, 2H), 2.05-1.87 (m, 8H), 1.79 (d, J = |
| | 12.0 Hz, 3H), 1.70-1.55 (m, 2H), 1.52-1.36 (m, 2H), 1.19-1.08 (m, 10H), |
| | 1.01 (d, J = 7.2 Hz, 3H), 0.97-0.82 (m, 6H) |
| 313 | ¹H NMR: (400 MHz, DMSO-d₆) δ: 10.87 (br d, J = 13.6 Hz, 1H), 8.16 (s, 1H), |
| | 7.95 (br d, J = 14.4 Hz, 2H), 7.81 (br d, J = 8.4 Hz, 1H), 7.68 (dd, J = 2.8, 8.8 |
| | Hz, 1H), 7.45 (s, 1H), 7.34 (t, J = 8.0 Hz, 1H), 6.91-6.77 (m, 2H), 5.08 (br s, |
| | 2H), 4.72-4.61 (m, 1H), 4.51-4.36 (m, 1H), 4.13-4.02 (m, 2H), 3.74-3.66 |
| | (m, 2H), 3.62 (br s, 3H), 3.33 (br d, J = 3.6 Hz, 2H), 3.29 (br s, 4H), 3.25-3.20 |
| | (m, 2H), 2.92-2.83 (m, 4H), 2.76 (s, 3H), 2.59 (br s, 2H), 2.15-1.69 (m, 11H), |
| | 1.37 (br dd, J = 6.8, 13.6 Hz, 5H), 1.22 (br s, 1H), 1.27-1.20 (m, 1H), 1.15- |
| | 1.05 (m, 2H), 0.92 (s, 6H), 0.87-0.77 (m, 2H) |
| 314 | ¹H NMR: (400 MHz, DMSO-d₆) δ: 11.16 (s, 1H), 8.17 (s, 1H), 8.11 (s, 1H), 8.00 |
| | (s, 1H), 7.92 (s, 1H), 7.83 (br d, J = 7.6 Hz, 1H), 7.46 (s, 1H), 7.34 (t, J = 8.0 |
| | Hz, 1H), 7.10-7.04 (m, 1H), 6.98 (d, J = 8.0 Hz, 1H), 6.82 (d, J = 7.6 Hz, 1H), |
| | 6.54 (d, J = 8.0 Hz, 1H), 5.62 (dd, J = 5.0, 12.8 Hz, 1H), 5.08 (s, 2H), 4.07- |
| | 3.97 (m, 2H), 3.75-3.57 (m, 2H), 3.39-3.32 (m, 2H), 3.30 (s, 2H), 3.27-3.21 |
| | (m, 1H), 3.07-2.99 (m, 2H), 2.98-2.79 (m, 6H), 2.78 (s, 3H), 2.77-2.65 (m, |
| | 2H), 2.39-2.33 (m, 2H), 2.29-2.08 (m, 4H), 2.01 (br d, J = 7.2 Hz, 2H), 1.93 |
| | (br d, J = 3.6 Hz, 4H), 1.79 (br d, J = 11.6 Hz, 2H), 1.61-1.52 (m, 2H), 1.47- |
| | 1.38 (m, 1H), 1.23-1.13 (m, 2H), 1.12 (s, 6H), 0.94-0.83 (m, 2H) |
| 315 | ¹H NMR: (400 MHz, DMSO-d₆) δ: 11.09-10.95 (m, 1H), 8.16 (s, 1H), 8.00 (s, |
| | 1H), 7.92 (s, 1H), 7.86-7.80 (m, 1H), 7.46 (s, 1H), 7.34 (t, J = 8.0 Hz, 1H), |
| | 7.14 (t, J = 8.0 Hz, 1H), 6.83 (d, J = 7.6 Hz, 1H), 6.63 (d, J = 8.0 Hz, 1H), |
| | 6.59-6.49 (m, 1H), 5.26-5.13 (m, 1H), 5.13-5.04 (m, 2H), 3.76-3.63 (m, 1H), |
| | 3.57 (br dd, J = 4.8, 8.8 Hz, 1H), 3.54 (br s, 2H), 3.36-3.33 (m, 2H), 3.30 (br |
| | s, 2H), 3.28-3.26 (m, 2H), 3.25-3.19 (m, 2H), 2.89 (s, 2H), 2.87 (br s, 2H), |
| | 2.81 (br s, 1H), 2.79 (s, 3H), 2.78-2.74 (m, 1H), 2.64-2.52 (m, 4H), 2.37 (br |
| | s, 2H), 2.23-2.11 (m, 2H), 2.01 (br d, J = 7.2 Hz, 2H), 1.96-1.85 (m, 5H), |
| | 1.79 (br d, J = 11.6 Hz, 2H), 1.58-1.48 (m, 2H), 1.46-1.38 (m, 1H), 1.25- |
| | 1.14 (m, 2H), 1.12 (s, 6H), 0.94-0.81 (m, 2H) |
| 316 | ¹H NMR: (400 MHz, DMSO-d₆) δ = 11.04 (br s, 1H), 8.16 (s, 1H), 8.00 (s, 1H), |
| | 7.91 (s, 1H), 7.83 (br d, J = 8.4 Hz, 1H), 7.46 (s, 1H), 7.34 (br t, J = 8.0 Hz, |
| | 1H), 6.97 (s, 1H), 6.82 (br d, J = 7.6 Hz, 1H), 6.80-6.72 (m, 2H), 5.17 (br d, J = |
| | 6.8 Hz, 1H), 5.08 (br s, 2H), 3.76-3.63 (m, 1H), 3.55 (br s, 3H), 3.39-3.36 |
| | (m, 2H), 3.30 (br s, 3H), 3.27-3.19 (m, 2H), 2.87 (br d, J = 8.0 Hz, 4H), 2.83 |
| | (br s, 1H), 2.78 (s, 3H), 2.77-2.72 (m, 2H), 2.58 (br d, J = 14.4 Hz, 2H), 2.36 |
| | (br s, 2H), 2.29-2.09 (m, 3H), 2.00 (br d, J = 6.8 Hz, 2H), 1.97-1.81 (m, 6H), |
| | 1.77 (br d, J = 14.0 Hz, 2H), 1.55-1.46 (m, 2H), 1.41 (br s, 1H), 1.17 (br s, |
| | 2H), 1.11 (s, 6H), 0.92-0.81 (m, 2H) |
| 317 | ¹H NMR: (400 MHz, DMSO-d₆) δ: 11.06 (s, 1H), 8.17 (s, 1H), 8.08-7.91 (m, |
| | 2H), 7.84 (d, J = 8.0 Hz, 1H), 7.45 (s, 1H), 7.35 (t, J = 8.0 Hz, 1H), 7.00-6.93 |
| | (m, 1H), 6.90-6.80 (m, 3H), 5.67-5.53 (m, 1H), 5.28 (dd, J = 5.6, 12.8 Hz, |
| | 1H), 5.12 (s, 2H), 3.50 (m, 1H), 3.30 (m, 2H), 3.26-3.20 (m, 4H), 3.07 (d, J = |
| | 11.2 Hz, 3H), 2.88 (d, J = 7.2 Hz, 6H), 2.83 (m, 2H), 2.79 (s, 3H), 2.71 (d, J = |
| | 10.8 Hz, 2H), 2.67 (m, 1H), 2.65-2.60 (m, 2H), 2.58 (m, 1H), 2.54 (s, 2H), |
| | 2.53-2.51 (m, 1H), 2.45 (m, 1H), 2.03-1.91 (m, 5H), 1.77 (d, J = 13.2 Hz, |
| | 4H), 1.73-1.55 (m, 6H), 1.47 (dd, J = 6.8, 10.8 Hz, 9H), 1.25-1.14 (m, 2H), |
| | 1.06-0.94 (m, 2H) |
| 318 | ¹H NMR: (400 MHz, DMSO-d₆) δ: 11.09 (s, 1H), 8.16 (s, 1H), 8.00 (s, 1H), |
| | 7.91 (s, 1H), 7.83 (d, J = 8.4 Hz, 1H), 7.46 (s, 1H), 7.34 (t, J = 8.0 Hz, 1H), |
| | 6.86-6.81 (m, 2H), 6.80-6.75 (m, 1H), 5.36-5.28 (m, 1H), 5.08 (s, 2H), 4.81- |
| | 4.71 (m, 1H), 3.58-3.52 (m, 1H), 3.31-3.27 (m, 4H), 3.26-3.19 (m, 2H), 3.15- |
| | 3.10 (m, 2H), 2.93-2.81 (m, 6H), 2.78 (s, 3H), 2.76-2.57 (m, 5H), 2.39- |
| | 2.33 (m, 2H), 2.03-1.98 (m, 3H), 1.97-1.86 (m, 5H), 1.81-1.75 (m, 2H), |
| | 1.61-1.54 (m, 2H), 1.47-1.36 (m, 8H), 1.21-1.13 (m, 2H), 1.11 (s, 6H), 0.92- |
| | 0.83 (m, 2H) |
| 319 | ¹H NMR: (400 MHz, DMSO-d₆) δ: 11.05 (s, 1H), 8.17 (s, 1H), 8.00 (s, 1H), |
| | 7.92 (s, 1H), 7.85-7.82 (m, 1H), 7.46 (s, 1H), 7.34 (t, J = 8.0 Hz, 1H), 7.28 (d, |
| | J = 2.8 Hz, 1H), 7.05-6.97 (m, 2H), 6.82 (d, J = 7.6 Hz, 1H), 6.50 (d, J = 7.2 |
| | Hz, 1H), 6.42 (d, J = 3.6 Hz, 1H), 5.59-5.53 (m, 1H), 5.09 (s, 2H), 3.62-3.58 |
| | (m, 1H), 3.45-3.38 (m, 2H), 3.31-3.28 (m, 4H), 3.27-3.21 (m, 2H), 2.92- |
| | 2.80 (m, 8H), 2.78 (s, 3H), 2.73-2.61 (m, 3H), 2.38-2.34 (m, 2H), 2.05-1.90 |
| | (m, 8H), 1.81-1.76 (m, 2H), 1.68-1.60 (m, 2H), 1.50-1.35 (m, 2H), 1.20- |
| | 1.14 (m, 2H), 1.12 (s, 6H), 0.92-0.84 (m, 2H) |
| 320 | ¹H NMR: (400 MHz, DMSO-d₆) δ = 11.06 (s, 1H), 8.17 (s, 1H), 7.91 (d, J = |
| | 19.6 Hz, 2H), 7.84 (dd, J = 1.6, 8.2 Hz, 1H), 7.48 (d, J = 1.6 Hz, 1H), 7.35 (t, |
| | J = 8.0 Hz, 1H), 7.00-6.92 (m, 1H), 6.91-6.80 (m, 3H), 5.70-5.55 (m, 1H), |
| | 5.29 (dd, J = 5.2, 12.4 Hz, 1H), 5.10 (s, 2H), 3.82-3.76 (m, 2H), 3.50-3.47 |
| | (m, 1H), 3.31 (s, 3H), 3.27-3.22 (m, 2H), 3.07 (d, J = 10.4 Hz, 2H), 2.97 (s, |
| | 4H), 2.89 (d, J = 7.6 Hz, 4H), 2.85-2.81 (m, 1H), 2.80 (s, 3H), 2.75-2.68 (m, |
| | 2H), 2.64-2.58 (m, 1H), 2.22 (s, 3H), 2.05-1.87 (m, 7H), 1.82-1.72 (m, |
| | 3H), 1.68 (s, 4H), 1.54-1.37 (m, 9H), 1.20-1.07 (m, 2H), 0.92-0.79 (m, 2H) |
| 321 | ¹H NMR: (400 MHz, DMSO-d₆) δ: 11.06 (s, 1 H), 8.17 (s, 1 H), 7.97 (s, 1 H), |
| | 7.92 (s, 1 H), 7.85 (d, J = 8.0 Hz, 1 H), 7.46 (s, 1 H), 7.36 (t, J = 8.0 Hz, 1 H), |
| | 6.93-7.00 (m, 1 H), 6.82-6.90 (m, 3 H), 5.55-5.70 (m, 1 H), 5.29 (m, 1 H), 5.11 |
| | (s, 2 H), 3.65 (s, 2 H), 3.48 (m, 3 H), 3.24 (d, J = 8.8 Hz, 2 H), 3.03-3.10 (m, 2 |
| | H), 2.93-3.01 (m, 1 H), 2.85-2.93 (m, 5 H), 2.81-2.85 (m, 1 H), 2.80 (s, 3 |
| | H), 2.68-2.73 (m, 2 H), 2.64 (d, J = 8.0 Hz, 1 H), 2.53-2.60 (m, 2 H), 2.32 (d, |
| | J = 4.8 Hz, 5 H), 1.91-2.04 (m, 5 H), 1.79 (d, J = 1.6 Hz, 1 H), 1.72 (s, 6 H), 1.47 |
| | (m, 9 H), 1.27-1.35 (m, 1 H), 1.08-1.18 (m, 2 H), 0.87-0.98 (m, 2 H) |
| 322 | ¹H NMR: (400 MHz, DMSO-d₆) δ: 11.08 (s, 1 H), 8.17 (s, 1 H), 8.02 (s, 1 H), |
| | 7.93 (s, 1 H), 7.84 (d, J = 8.0 Hz, 1 H), 7.47 (s, 1 H), 7.36 (t, J = 8.0 Hz, 1 H), 6.95- |
| | 7.01 (m, 1 H), 6.84-6.92 (m, 3 H), 5.35 (dd, J = 12.4, 5.2 Hz, 1 H), 5.10 (s, 2 |
| | H), 3.68 (d, J = 7.6 Hz, 1 H), 3.63 (s, 3 H), 3.45-3.56 (m, 1 H), 3.35-3.41 (m, |
| | 1 H), 3.34 (s, 1 H), 3.18-3.29 (m, 2 H), 3.03-3.12 (m, 2 H), 2.89-2.93 (m, 3 |
| | H), 2.88 (s, 2 H), 2.85 (s, 1 H), 2.80 (s, 4 H), 2.71-2.78 (m, 2 H), 2.67-2.71 |
| | (m, 1 H), 2.65 (s, 1 H), 2.52-2.55 (m, 2 H), 2.36-2.41 (m, 1 H), 2.10-2.26 |
| | (m, 2 H), 1.94-2.05 (m, 6 H), 1.79 (d, J = 9.2 Hz, 3 H), 1.49-1.63 (m, 2 H), |
| | 1.36-1.48 (m, 1 H), 1.26 (s, 2 H), 1.13 (s, 6 H), 0.84-1.02 (m, 2 H) |
| 323 | ¹H NMR: (400 MHz, DMSO-d₆) δ: 11.07 (s, 1H), 8.17 (s, 1H), 7.99 (s, 1H), |
| | 7.95 (s, 1H), 7.87-7.82 (m, 1H), 7.47 (s, 1H), 7.35 (t, J = 8.0 Hz, 1H), 6.99- |
| | 6.94 (m, 1H), 6.88 (d, J = 8.0 Hz, 1H), 6.83 (d, J = 7.2 Hz, 2H), 5.69-5.57 (m, |
| | 1H), 5.33-5.25 (m, 1H), 5.10 (s, 2H), 3.76 (s, 2H), 3.53-3.46 (m, 1H), 3.31 |
| | (s, 2H), 3.27-3.20 (m, 2H), 3.11-3.02 (m, 2H), 2.88 (m, 5H), 2.84 (m, 2H), |
| | 2.79 (s, 3H), 2.75-2.60 (m, 5H), 2.59-2.54 (m, 3H), 2.32-2.26 (m, 2H), |
| | 2.04-1.91 (m, 5H), 1.88-1.74 (m, 4H), 1.61-1.54 (m, 1H), 1.52-1.42 (m, 8H), |
| | 1.29 (m, 1H), 1.18-1.08 (m, 2H), 0.95 (d, J = 6.0 Hz, 3H), 0.91-0.81 (m, 2H) |
| 324 | ¹H NMR: (400 MHz, DMSO-d₆) δ: 11.26 (s, 1 H), 8.18 (s, 1 H), 8.01 (s, 1 H), |
| | 7.93 (s, 1 H), 7.84 (d, J = 8.8 Hz, 1 H), 7.51-7.79 (m, 1 H), 7.47 (s, 1 H), 7.35 |
| | (t, J = 8.0 Hz, 1 H), 7.03-7.11 (m, 1 H), 6.95 (d, J = 8.0 Hz, 1 H), 6.83 (d, J = 8.0 |
| | Hz, 1 H), 6.62 (d, J = 8.0 Hz, 1 H), 5.61 (m, 1 H), 5.09 (s, 2 H), 3.84-3.93 (m, |
| | 2 H), 3.58-3.63 (m, 1 H), 3.43 (d, J = 5.6 Hz, 2 H), 3.30 (s, 2 H), 3.19-3.28 (m, |
| | 2 H), 3.01 (t, J = 10.0 Hz, 2 H), 2.91-2.97 (m, 1 H), 2.85-2.91 (m, 4 H), 2.78 |
| | (s, 3 H), 2.71 (s, 1 H), 2.55-2.63 (m, 1 H), 2.47-2.50 (m, 1 H), 2.37 (s, 2 H), |
| | 2.08-2.31 (m, 4 H), 2.02 (d, J = 7.2 Hz, 2 H), 1.93 (s, 4 H), 1.79 (d, J = 12.0 Hz, |
| | 2 H), 1.51-1.61 (m, 2 H), 1.38-1.47 (m, 1 H), 1.14-1.24 (m, 2 H), 1.12 (s, 6 |
| | H), 0.83-0.95 (m, 2 H) |
| 325 | ¹H NMR: (400 MHz, DMSO-d₆) δ: 11.18 (s, 1 H), 8.03-8.25 (m, 2 H), 8.01 (s, |
| | 1 H), 7.93 (s, 1 H), 7.84 (m, 1 H), 7.47 (s, 1 H), 7.35 (t, J = 8.0 Hz, 1 H), 7.13- |
| | 7.21 (m, 1 H), 7.07 (d, J = 8.4 Hz, 1 H), 7.02 (d, J = 7.6 Hz, 1 H), 6.83 (d, J = 7.6 |
| | Hz, 1 H), 5.41 (m, 1 H), 5.09 (s, 2 H), 3.53-3.88 (m, 2 H), 3.40-3.53 (m, 2 |
| | H), 3.30 (s, 2 H), 3.19-3.28 (m, 2 H), 3.01 (d, J = 11.6 Hz, 2 H), 2.92 (s, 1 H), |
| | 2.84-2.90 (m, 4 H), 2.78 (s, 3 H), 2.76 (s, 1 H), 2.70-2.75 (m, 1 H), 2.64- |
| | 2.69 (m, 1 H), 2.37 (d, J = 0.8 Hz, 2 H), 2.15-2.30 (m, 2 H), 2.09 (m, 2 H), 2.01 |
| | (d, J = 7.2 Hz, 2 H), 1.96 (s, 4 H), 1.79 (d, J = 11.6 Hz, 3 H), 1.28-1.72 (m, 3 H), |
| | 1.14-1.24 (m, 2 H), 1.12 (s, 6 H), 0.81-0.96 (m, 2 H) |
| 326 | ¹H NMR: (400 MHz, DMSO-d₆) δ: 11.07 (s, 1H), 8.18 (s, 1H), 7.98 (d, J = 19.6 |
| | Hz, 2H), 7.85 (d, J = 7.6 Hz, 1H), 7.47 (s, 1H), 7.35 (t, J = 8.0 Hz, 1H), 7.02- |
| | 6.92 (m, 1H), 6.92-6.81 (m, 3H), 5.70-5.53 (m, 1H), 5.29 (dd, J = 5.2, 12.8 |
| | Hz, 1H), 5.11 (s, 2H), 3.84-3.76 (m, 2H), 3.57-3.46 (m, 1H), 3.31 (s, 3H), |
| | 3.28-3.20 (m, 2H), 3.07 (d, J = 11.2 Hz, 2H), 2.92 (s, 1H), 2.89 (d, J = 7.6 Hz, |
| | 6H), 2.84 (s, 1H), 2.79 (s, 3H), 2.71 (s, 2H), 2.64 (d, J = 8.8 Hz, 2H), 2.60-2.52 |
| | (m, 4H), 2.05-1.90 (m, 5H), 1.89-1.69 (m, 4H), 1.68-1.50 (m, 2H), 1.48 (dd, |
| | J = 6.8, 10.4 Hz, 7H), 1.35-1.26 (m, 1H), 1.19-1.07 (m, 2H), 0.98 (d, J = 6.0 |
| | Hz, 3H), 0.92-0.78 (m, 2H). |
| 327 | ¹H NMR: (400 MHz, DMSO-d₆) δ: 11.08 (s, 1 H), 8.18 (s, 1 H), 7.87-8.09 (m, |
| | 2 H), 7.85 (m, 1 H), 7.46 (s, 1 H), 7.36 (t, J = 8.0 Hz, 1 H), 6.95-7.00 (m, 1 H), |
| | 6.83-6.90 (m, 3 H), 5.56-5.66 (m, 1 H), 5.29 (m, 1 H), 5.13 (s, 2 H), 4.08- |
| | 4.35 (m, 4 H), 3.75-3.99 (m, 2 H), 3.45-3.55 (m, 2 H), 3.31 (br s, 2 H), 3.20- |
| | 3.27 (m, 2 H), 3.08 (br d, J = 12.0 Hz, 2 H), 2.91-3.02 (m, 3 H), 2.86-2.90 |
| | (m, 4 H), 2.84 (br s, 1 H), 2.80 (s, 3 H), 2.73 (br s, 1 H), 2.65-2.71 (m, 2 H), |
| | 2.63 (br s, 1 H), 2.52-2.58 (m, 1 H), 1.92-2.02 (m, 5 H), 1.76-1.83 (m, 1 H), |
| | 1.66-1.72 (m, 2 H), 1.50 (br s, 3 H), 1.43-1.48 (m, 6 H), 1.11-1.21 (m, 2 H), |
| | 0.94-1.03 (m, 2 H). |
| 328 | ¹H NMR: (400 MHz, DMSO-d₆) δ: 11.14 (s, 1H), 8.18 (s, 1H), 8.01 (s, 1H), |
| | 7.93 (s, 1H), 7.84 (d, J = 8.0 Hz, 1H), 7.47 (s, 1H), 7.35 (t, J = 8.0 Hz, 1H), 6.83 |
| | (d, J = 7.6 Hz, 1H), 6.76-6.68 (m, 1H), 5.52-5.44 (m, 1H), 5.10 (s, 2H), 4.89- |
| | 4.75 (m, 1H), 3.88-3.63 (m, 1H), 3.59-3.54 (m, 1H), 3.31 (s, 3H), 3.27- |
| | 3.22 (m, 1H), 3.17-3.11 (m, 2H), 3.02-2.93 (m, 1H), 2.91-2.86 (m, 4H), 2.79 |
| | (s, 3H), 2.77-2.71 (m, 2H), 2.69-2.55 (m, 2H), 2.41-2.34 (m, 2H), 2.28- |
| | 2.06 (m, 5H), 2.04-1.98 (m, 2H), 1.93 (t, J = 10.8 Hz, 5H), 1.82-1.76 (m, 2H), |
| | 1.62-1.55 (m, 2H), 1.44 (d, J = 6.4 Hz, 6H), 1.41-1.37 (m, 1H), 1.22-1.15 |
| | (m, 2H), 1.13 (s, 6H), 0.94-0.84 (m, 2H). |

### Biological Assays

### Target Protein Degradation

### Cbl-b Degradation Method A

| Material/Reagent | Vendor | Catalog # |
|---|---|---|
| DMSO | Sigma | |
| RPMI 1640 Medium, GlutaMAX ™ | Gibco | 61870-036 |
| Supplement | | |
| Penstrep | Gibco | 15140-122 |
| Fetal Bovine Serum, qualified | Gibco | 26140-079 |
| 8-well PCR strips | | |
| 96-well Polyproplyene plate | Grenier Bio- | 650201 |
| | One | |
| 96-well deep well plate | | |
| DPBS, no calcium, no magnesium | Gibco | 14190-144 |
| Trypan blue | Invitrogen | |
| Countess slide | Invitrogen | |
| 96-well cell culture flat bottom | Celltreat | 229196 |
| 96-well v-bottom | Nunc | 249570 |
| Fixiative | Biolegend | 420801 |
| Perm III | BD | 558050 |
| anti CBLb antibody | Cell Signaling | 9498S |
| 10X Perm Wash buffer | BD | 554723 |
| a-Rabbit-AF488 | Cell Signaling | 4412S |
| CS&T beads | BD | 642412 |

Cbl-b-HiBit KI Jurkat Cells were purchased from Promega, and cell media consisted of RPMI 1640 with 10% FBS and 1% pen/strep. The cells were counted, measured for viability, and 100 μL of the cell solution was into 96 well flat bottom plates at a ratio of 290,000 cells per 100 μL. Compounds were dissolved in DMSO to make a 10 mM stock solution, which was diluted with cell media. 100 μL of the diluted compound mixture was added to the wells. The plate was incubated for at least 20 hours at 37° C. and 5% CO₂. After incubation, the plate was shaken at 700-750 RMP for 15-30 minutes to ensure cells were removed from the wells. The wells were transferred to a 96-well v-bottom plate and spun for 2200 RPM for 3 minutes. The supernatant was removed, cells re-suspended in 100 μL of the fixative, and subsequently incubated at room temperature for 15 minutes. Afterwards, 150 μL of DPBS was added to each well, and the cells were centrifuged at 2500 RPM for 3 minutes. The supernatant was removed and the plate was chilled on ice for 2-3 minutes. While still on the ice, 100 μL of Perm III was added to each well, the wells mixed, and subsequently left on the ice for another 15 minutes. Afterwards, the plate was removed from the ice, 150 μL of DBPS was added to each well, and the cells centrifuged at 2700 RPM for 3 minutes. The supernatant was removed, and an additional 200 μL of was DPBS was added to each well, followed by centrifuging at 2700 RPM for 3 minutes, after which the supernatant was removed. The anti-Cbl-b antibody was diluted 1:200 in 1× Perm Wash buffer and 50 μL of the antibody mixture was added to each well and mixed. After addition, the plate was incubated for 35-45 minutes at room temperature. After incubation, 200 μL of DPBS was added to each wells, and the plate centrifuged at 2700 RPM for 3 minutes, followed by the removal of the supernatant. The washing step was repeated by adding 200 μL of DPBS to each wells followed by centrifuge at 2700 RPM for 3 minutes and removal of the supernatant. The a-Rabbit-AF488 antibody was diluted 1:1000 in Perm Wash buffer, 50 μL of the dilution added to each well, and mixed. After mixing, the plate was incubated for 30 minutes at room temperature in the dark. Subsequently, the supernatant was removed, and 200 μL of DBPS added to the cells, which were then centrifuged at 2900 RPM for 4 minutes. The supernatant was removed, and an additional 95 μL of DBPS was added to the wells and mixed. The cells were then transferred to a round bottom polypropylene plate and analyzed using FACS with the parameters below:

| | |
|---|---|
| Sample Flow | 1 |
| Sample volume | 85 |
| Mixing volume | 50 |
| Mixing speed | 200 |
| # of mixes | 2 |
| Wash | 200 |

### Cbl-b Degradation Method B

| Reagent/Equipment | Vendor | Catalogue # |
|---|---|---|
| Cbl-b-HiBit Jurkat cells | Promega | Promega |
| | | CS3023199 |
| Recovery Cell culture Freezing | Gibco | Gibco 12648-010 |
| media- | | |
| RPMI-1640 | ATCC or Gibco 22400- | 30-2004 |
| | 089 | |
| Corning ® CoolCell | Corning | |
| FBS-Fetal Bovine Serum | Gibco | 261-40-079 |
| 0.25% Trypsin-EDTA (Gibco,) | Gibco | 25200-056 |
| Pen/Strep | Gibco | 15140-122 |
| DPBS (1X) | Gibco | 14190-144 |
| Trypan Blue Solution, 0.4% | Thermo Fisher, | 15250061 |
| 50 mL Conical Tubes | Thomas Scientific | 1222B18 |
| 250 mL Centrifuge Tubes | Corning | 21008-771 |
| Nano-Glo ® HiBiT Lytic | Promega | N3050 |
| Detection System, 10 × 100 mL | | |
| includes: | | |
| Nano-Glo ® HiBiT Lytic | | |
| Substrate | | |
| Nano-Glo ® HiBiT Lytic | | |
| Buffer | | |
| LgBiT Protein | | |
| White 384-well solid bottom | Corning | 3570 |
| 1.5 mL Effendorf Tubes | VWR | 20901-551 |
| Combi Standard Cassette | Thermo Fisher | 24072675 |
| Bravo 384-well 30 uL tips | (Fluotics,) | AGI-30.NS |
| MASTERBLOCK ® 384-Well Deep | VWR | VWR001 |
| Well Microplates, Greiner Bio-One - | | |
| Sterile | | |
| Cell Treat T182 Flask | | |

Cbl-b-HiBit KI Jurkat Cells were purchased from Promega, and cell media consisted of RPMI 1640 with 10% PBS and 1% pen/strep. The cells were counted, measured for viability, and 35 μL of the cell solution was seeded into 384 well flat bottom plates at a ratio of 20,000 cells per 35 μL. After which, the plates were incubated for ˜24 hours at 37° C. and 5% CO₂. Compounds were ordered from Evotec in a 384 well plate at a 1 mM top concentration in DMSO and serially diluted 1:3 in DMSO for an 11-point dose response. Using the Bravo instrument, 1 uL of compound solution or DMSO was transferred to 125 uL of media. Each well was treated with 5 μL of compound plus media mixture or DMO and subsequently incubated for at least 6-24 hours at 37° C. and 5% CO₂. After incubation, 30 μL of either NanoGlo media was added to each cell, and the plates read on the Synergy Neo2 machine.

The concentration of an exemplary compound that leads to half maximal degradation (DC₅₀) as well as the maximum degradation observed (Dₘₐₓ, conventionally expressed as a percentage of control) is shown below in Table 2.

TABLE 2

| | Cbl-b | Cbl-b | Cbl-b | Cbl-b |
|---|---|---|---|---|
| | Degredation | Degredation | Degredation | Degredation |
| Cmp. | Method A | Method A | Method B | Method B |
| No. | Dₘₐₓ (%) | IC₅₀ (nM) | Dₘₐₓ (%) | IC₅₀ (nM) |
| 1 | B | A | B | A |
| 2 | A | A | B | A |
| 3 | A | A | B | A |
| 4 | B | A | B | A |
| 5 | A | A | B | A |
| 6 | A | A | NT | NT |
| 7 | A | A | NT | NT |
| 8 | A | B | B | A |
| 9 | B | A | B | A |
| 10 | A | A | A | A |
| 11 | C | B | C | B |
| 12 | NT | NT | A | A |
| 13 | NT | NT | B | A |
| 14 | NT | NT | B | A |
| 15 | B | C | NT | D |
| 16 | A | C | C | B |
| 17 | NT | D | NT | D |
| 18 | C | C | NT | D |
| 19 | NT | C | C | A |
| 20 | NT | D | NT | D |
| 21 | NT | D | NT | D |
| 22 | B | B | B | A |
| 23 | C | C | NT | D |
| 24 | NT | NT | A | A |
| 25 | C | B | NT | NT |
| 26 | NT | NT | NT | D |
| 27 | NT | NT | NT | D |
| 28 | NT | D | NT | D |
| 29 | NT | D | NT | D |
| 30 | NT | D | NT | D |
| 31 | NT | D | NT | D |
| 32 | NT | NT | A | C |
| 33 | NT | NT | NT | D |
| 34 | NT | NT | NT | D |
| 35 | NT | NT | NT | D |
| 36 | NT | NT | NT | D |
| 37 | NT | NT | NT | D |
| 38 | NT | NT | NT | D |
| 39 | NT | NT | NT | D |
| 40 | NT | NT | NT | D |
| 41 | NT | NT | A | B |
| 42 | NT | NT | NT | D |
| 43 | NT | NT | C | B |
| 44 | NT | NT | NT | D |
| 45 | NT | NT | NT | D |
| 46 | NT | NT | NT | D |
| 47 | NT | NT | NT | D |
| 48 | NT | NT | B | C |
| 49 | NT | NT | NT | D |
| 50 | NT | NT | NT | NT |
| 51 | NT | NT | NT | D |
| 52 | NT | NT | NT | D |
| 53 | NT | NT | NT | D |
| 54 | NT | NT | NT | D |
| 55 | NT | NT | NT | D |
| 56 | NT | NT | NT | D |
| 57 | NT | NT | NT | D |
| 58 | NT | NT | NT | D |
| 59 | NT | NT | NT | D |
| 60 | NT | NT | NT | D |
| 61 | NT | NT | NT | D |
| 62 | NT | NT | NT | D |
| 63 | NT | NT | NT | D |
| 64 | NT | NT | NT | D |
| 65 | NT | NT | NT | D |
| 66 | NT | NT | NT | D |
| 67 | NT | NT | NT | D |
| 68 | NT | NT | NT | D |
| 69 | NT | NT | NT | D |
| 70 | NT | NT | NT | D |
| 71 | NT | NT | NT | D |
| 72 | NT | NT | B | A |
| 73 | NT | NT | B | B |
| 74 | A | B | A | A |
| 75 | NT | D | NT | D |
| 76 | NT | D | NT | D |
| 77 | NT | D | NT | D |
| 78 | NT | NT | NT | D |
| 79 | NT | D | NT | D |
| 80 | NT | NT | NT | D |
| 81 | NT | D | NT | D |
| 82 | NT | D | NT | D |
| 83 | NT | D | NT | D |
| 84 | NT | D | NT | D |
| 85 | NT | B | A | B |
| 86 | NT | D | NT | D |
| 87 | NT | C | NT | D |
| 88 | NT | D | NT | D |
| 89 | NT | D | NT | D |
| 90 | C | D | NT | D |
| 91 | NT | D | NT | D |
| 92 | NT | D | NT | D |
| 93 | B | B | C | A |
| 94 | B | B | B | A |
| 95 | NT | C | NT | D |
| 96 | NT | D | NT | D |
| 97 | A | B | B | A |
| 98 | NT | D | NT | D |
| 99 | NT | D | NT | D |
| 100 | C | D | NT | D |
| 101 | NT | D | NT | D |
| 102 | NT | D | NT | D |
| 103 | B | D | NT | D |
| 104 | NT | C | NT | D |
| 105 | NT | D | NT | D |
| 106 | A | C | NT | D |
| 107 | C | B | NT | D |
| 108 | C | B | B | B |
| 109 | A | A | NT | NT |
| 110 | C | B | NT | D |
| 111 | NT | C | NT | D |
| 112 | B | B | B | A |
| 113 | NT | NT | B | A |
| 114 | C | B | C | B |
| 115 | B | B | B | B |
| 116 | NT | D | NT | D |
| 117 | B | A | B | A |
| 118 | NT | D | NT | D |
| 119 | C | B | C | A |
| 120 | B | B | NT | NT |
| 121 | NT | B | NT | D |
| 122 | NT | C | NT | D |
| 123 | B | C | NT | D |
| 124 | NT | D | NT | D |
| 125 | NT | D | NT | D |
| 126 | NT | D | NT | D |
| 127 | NT | D | NT | D |
| 128 | NT | D | NT | D |
| 129 | NT | C | NT | D |
| 130 | NT | D | NT | D |
| 131 | B | B | B | A |
| 132 | B | B | B | A |
| 133 | A | B | NT | NT |
| 134 | C | C | NT | D |
| 135 | NT | C | NT | D |
| 136 | B | B | B | B |
| 137 | B | B | B | B |
| 138 | B | B | C | A |
| 139 | B | B | B | B |
| 140 | NT | NT | NT | NT |
| 141 | NT | NT | NT | NT |
| 142 | NT | NT | NT | NT |
| 143 | NT | NT | NT | NT |
| 144 | NT | NT | NT | NT |
| 145 | NT | NT | NT | NT |
| 146 | NT | NT | C | B |
| 147 | NT | NT | NT | D |
| 148 | A | A | A | A |
| 149 | NT | NT | B | B |
| 150 | NT | NT | NT | D |
| 151 | NT | NT | B | B |
| 152 | NT | NT | B | A |
| 153 | NT | NT | NT | NT |
| 154 | NT | NT | NT | D |
| 155 | NT | NT | NT | D |
| 156 | NT | NT | NT | D |
| 157 | A | A | B | A |
| 158 | A | A | B | A |
| 159 | A | A | B | A |
| 160 | B | A | B | A |
| 161 | C | B | NT | D |
| 162 | A | A | A | A |
| 163 | A | A | A | A |
| 164 | NT | NT | A | A |
| 165 | A | A | A | A |
| 166 | A | A | A | A |
| 167 | A | A | B | A |
| 168 | NT | NT | B | A |
| 169 | A | B | A | A |
| 170 | NT | NT | NT | D |
| 171 | A | B | B | A |
| 172 | B | B | B | B |
| 173 | A | B | B | A |
| 174 | A | B | A | A |
| 175 | A | B | B | A |
| 176 | B | B | B | A |
| 177 | B | B | B | A |
| 178 | B | B | B | A |
| 179 | B | B | B | A |
| 180 | NT | NT | C | A |
| 181 | B | A | B | A |
| 182 | NT | NT | C | B |
| 183 | NT | NT | C | B |
| 184 | A | A | NT | NT |
| 185 | B | B | B | B |
| 186 | B | B | B | A |
| 187 | B | B | NT | NT |
| 188 | B | B | B | A |
| 189 | B | B | NT | NT |
| 190 | A | B | NT | NT |
| 191 | NT | NT | B | A |
| 192 | NT | NT | B | A |
| 193 | A | A | B | A |
| 194 | A | A | B | A |
| 195 | A | A | B | A |
| 196 | A | A | B | A |
| 197 | NT | NT | NT | D |
| 198 | NT | NT | C | B |
| 199 | A | A | B | A |
| 200 | NT | NT | NT | D |
| 201 | NT | NT | NT | D |
| 202 | NT | NT | NT | D |
| 203 | B | A | B | A |
| 204 | A | A | B | A |
| 205 | B | A | NT | NT |
| 206 | NT | NT | NT | D |
| 207 | NT | NT | NT | D |
| 208 | B | B | B | A |
| 209 | B | B | B | A |
| 210 | NT | NT | NT | D |
| 211 | C | A | NT | D |
| 212 | B | B | B | A |
| 213 | NT | NT | NT | D |
| 214 | B | B | B | A |
| 215 | A | A | B | A |
| 216 | NT | NT | C | A |
| 217 | B | B | B | A |
| 218 | NT | NT | B | B |
| 219 | A | A | A | A |
| 220 | A | B | A | A |
| 221 | A | A | A | A |
| 222 | NT | NT | B | A |
| 223 | B | B | B | A |
| 224 | A | B | NT | NT |
| 225 | NT | NT | B | A |
| 226 | NT | NT | B | A |
| 227 | NT | NT | A | A |
| 228 | NT | NT | A | A |
| 229 | NT | NT | A | A |
| 230 | NT | NT | NT | NT |
| 231 | NT | NT | NT | NT |
| 232 | A | B | B | A |
| 233 | NT | NT | NT | D |
| 234 | B | B | B | A |
| 235 | NT | NT | B | A |
| 236 | A | A | A | A |
| 237 | A | A | A | A |
| 238 | B | B | B | A |
| 239 | NT | NT | A | A |
| 240 | NT | NT | NT | D |
| 241 | NT | NT | NT | NT |
| 242 | NT | NT | A | A |
| 243 | NT | NT | A | A |
| 244 | NT | NT | A | A |
| 245 | NT | NT | A | A |
| 246 | NT | NT | B | A |
| 247 | NT | NT | A | A |
| 248 | NT | NT | NT | D |
| 249 | NT | NT | A | A |
| 250 | NT | NT | B | A |
| 251 | NT | NT | A | B |
| 252 | NT | NT | B | B |
| 253 | NT | NT | NT | D |
| 254 | NT | NT | A | B |
| 255 | NT | NT | NT | D |
| 256 | NT | NT | NT | D |
| 257 | NT | NT | NT | D |
| 258 | NT | NT | NT | D |
| 259 | NT | NT | NT | D |
| 260 | NT | NT | NT | D |
| 261 | NT | NT | NT | D |
| 262 | NT | NT | C | B |
| 263 | NT | NT | A | A |
| 264 | NT | NT | A | A |
| 265 | NT | NT | C | B |
| 266 | NT | NT | A | A |
| 267 | NT | NT | A | A |
| 268 | NT | NT | A | A |
| 269 | NT | NT | A | A |
| 270 | NT | NT | A | A |
| 271 | NT | NT | B | B |
| 272 | NT | NT | B | B |
| 273 | NT | NT | D | A |
| 274 | NT | NT | A | A |
| 275 | NT | NT | B | B |
| 276 | C | B | C | B |
| 277 | C | B | C | A |
| 278 | B | C | C | B |
| 279 | NT | NT | A | D |
| 280 | B | B | C | A |
| 281 | B | B | C | A |
| 282 | NT | NT | A | D |
| 283 | B | C | C | B |
| 284 | B | C | C | A |
| 285 | NT | NT | A | D |
| 286 | B | C | C | A |
| 287 | NT | NT | A | D |
| 288 | NT | NT | A | D |
| 289 | B | B | C | A |
| 290 | NT | NT | A | D |
| 291 | NT | NT | A | D |
| 292 | C | B | A | C |
| 294 | A | C | C | A |
| 295 | NT | NT | A | D |
| 297 | NT | NT | A | D |
| 298 | NT | NT | A | D |
| 299 | NT | NT | A | D |
| 300 | B | C | C | A |
| 301 | A | C | C | A |
| 302 | B | C | C | B |
| 303 | B | B | C | A |
| 304 | B | C | C | A |
| 305 | NT | NT | A | D |
| 306 | B | C | C | A |
| 307 | B | C | C | A |
| 308 | NT | NT | A | D |
| 309 | NT | NT | A | D |
| 310 | B | B | C | B |
| 311 | B | C | C | A |
| 312 | B | C | C | A |
| 313 | NT | NT | A | D |
| 314 | B | B | B | C |
| 315 | B | C | C | A |
| 316 | A | C | C | A |
| 317 | B | C | C | A |
| 318 | B | C | C | A |
| 320 | A | C | C | A |
| 321 | C | B | C | A |
| 322 | A | C | C | B |
| 323 | B | B | C | A |
| 324 | B | B | C | A |
| 325 | B | C | C | B |
| 326 | B | C | C | A |
| 327 | B | B | C | A |

*DC₅₀ (nM): D > 500; 50 < C ≤ 500; 5 < B ≤ 50; A ≤ 5
**D_{Max} (%): C ≤ 35; 35 < B < 70; A ≤ 70
NT = not tested

While we have described a number of embodiments, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

What is claimed is:

1. A compound having the chemical structure I: 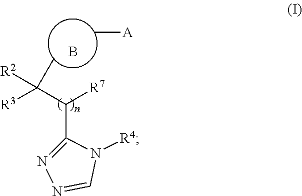
or a pharmaceutically acceptable salt thereof, wherein:
A is 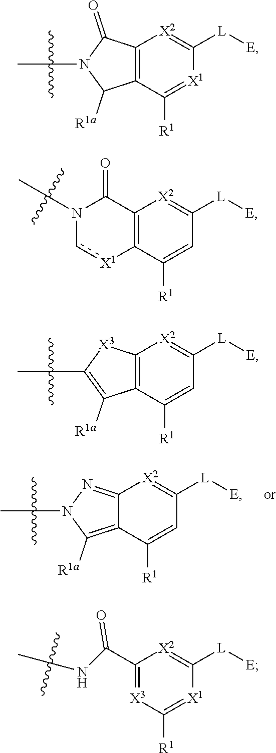
Ring B is phenyl or a 5 to 6-membered monocyclic heteroaryl;
X¹, X², and X³ are each independently CH or N;
X³ is O or S;
each R^{1a} is independently hydrogen or optionally substituted C₁-C₄ alkyl;
R¹ is selected from hydrogen, optionally substituted C₁-C₄ alkyl and optionally substituted (C₃-C₅)cycloalkyl;
R² and R³ are each independently selected from hydrogen, optionally substituted C₁-C₄ alkyl, optionally substituted cycloalkyl, and optionally substituted heterocyclyl; or R² and R³ are taken together to form optionally substituted (C₃-C₄)cycloalkyl or optionally substituted 4-6 membered heterocyclyl;
R⁷ is hydrogen when n is 1, 2, or 3; or R² is hydrogen and R⁷ and R³, together with the atoms to which they are attached, form optionally substituted (C₃-C₄)cycloalkyl when n is 1;
R⁴ is selected from hydrogen, optionally substituted C₁-C₄ alkyl and optionally substituted (C₃-C₆)cycloalkyl;
n is 0, 1, 2, or 3;
L is a chemical linking moiety; and
E is a cereblon E3 ligase-binding moiety represented by the chemical structure: 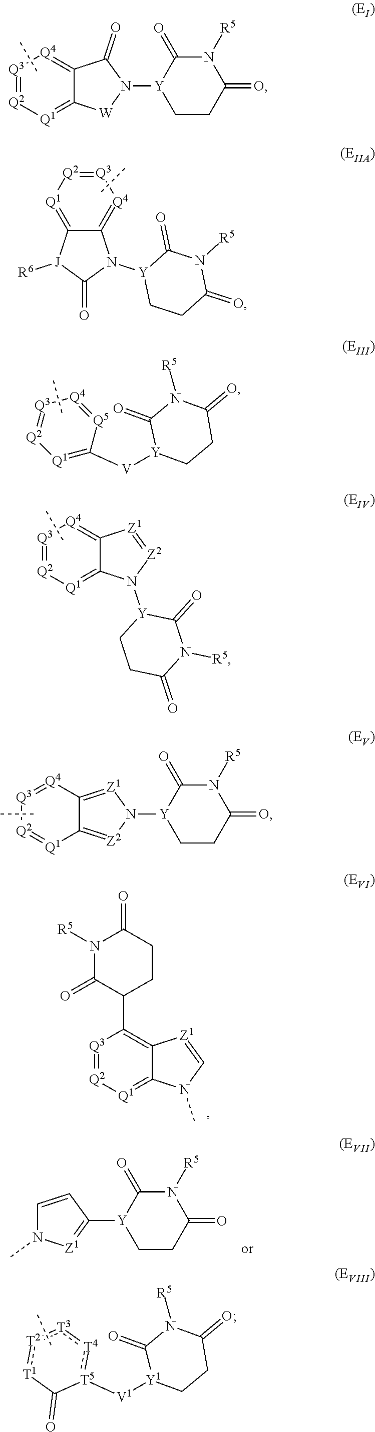
wherein:
W is CH₂, CHR^{v}, SO₂, or C(O);
Y and Y¹ are each independently N or CH;
J is CH or N;
Q¹, Q², Q³, Q⁴, Q⁵, T¹, T², T³, T⁴, and T⁵ are each independently CH, CR^{w}, N, or NR^{w};
Z¹ and Z² are each independently CH, CR^{x} or N;
V is absent or is NRⁿ or C(O)NR^{z};
V¹ is absent or C₁₋₄ alkylene;
R⁵ is H;
R⁶ is hydrogen or optionally substituted C₁₋₄ alkyl, C₁₋₄ haloalkyl, or C₃₋₄cycloalkyl;
R^{v} and R^{x} are each independently selected from halo, optionally substituted C₁₋₄ alkyl, optionally substituted C₁₋₄ alkoxy, cyano, OH, —NH (optionally substituted C₁₋₄ alkyl), and —NH (optionally substituted C₁₋₄ alkyl)₂;
R^{w} is absent, or selected from halo, optionally substituted C₁₋₄ alkyl, optionally substituted C₁₋₄ alkoxy, cyano, OH, —NH (optionally substituted C₁₋₄ alkyl), and —NH (optionally substituted C₁₋₄ alkyl)₂;
Rⁿ and R^{z} are each independently hydrogen or optionally substituted C₁₋₆ alkyl, optionally substituted cycloalkyl, or optionally substituted heterocyclyl; and
the dashed line indicates the part of the structure to which the chemical linking moiety (L) is attached.

2. The compound of claim 1, wherein:
or a pharmaceutically acceptable salt thereof, wherein:
A is 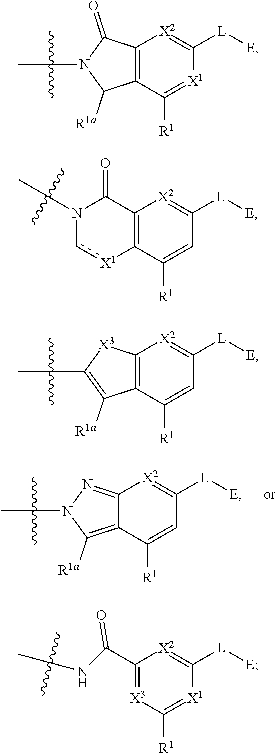
Ring B is phenyl or a 5 to 6-membered monocyclic heteroaryl;
X¹, X², and X³ are each independently CH or N;
X³ is O or S;
each R^{1a} is independently hydrogen or optionally substituted C₁-C₄ alkyl;
R¹ is selected from hydrogen, optionally substituted C₁-C₄ alkyl and optionally substituted (C₃-C₅)cycloalkyl;
R² and R³ are each independently selected from hydrogen, optionally substituted C₁-C₄ alkyl, optionally substituted cycloalkyl, and optionally substituted heterocyclyl; or R² and R³ are taken together to form optionally substituted (C₃-C₄)cycloalkyl or optionally substituted 4-6 membered heterocyclyl;
R⁷ is hydrogen when n is 1, 2, or 3; or R² is hydrogen and R⁷ and R³, together with the atoms to which they are attached, form optionally substituted (C₃-C₄)cycloalkyl when n is 1;
R⁴ is selected from hydrogen, optionally substituted C₁-C₄ alkyl and optionally substituted (C₃-C₆)cycloalkyl;
n is 0, 1, 2, or 3;
L is a chemical linking moiety; and
E is a cereblon E3 ligase-binding moiety represented by the chemical structure: 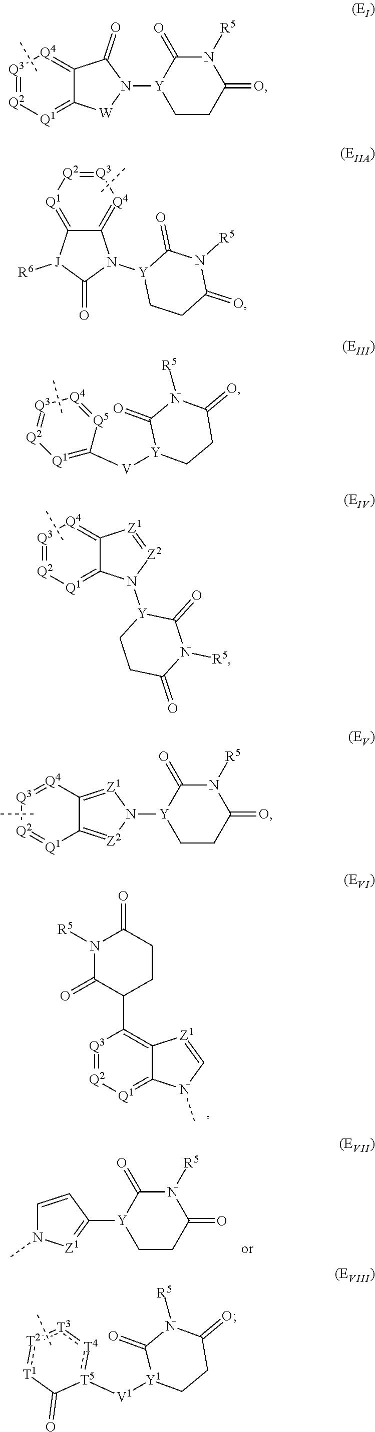
wherein:
W is CH₂, CHR^{v}, SO₂, or C(O);
Y and Y¹ are each independently N or CH;
J is CH or N;
Q¹, Q², Q³, Q⁴, Q⁵, T¹, T², T³, T⁴, and T⁵ are each independently CH, CR^{w}, N, or NR^{w};
Z¹ and Z² are each independently CH, CR^{x} or N;
V is absent or is NRⁿ or C(O)NR^{z};
V¹ is absent or C₁₋₄ alkylene;
R⁵ is H;
R⁶ is hydrogen or optionally substituted C₁₋₄ alkyl;
R^{v} and R^{x} are each independently selected from halo, optionally substituted C₁₋₄ alkyl, optionally substituted C₁₋₄ alkoxy, cyano, OH, —NH (optionally substituted C₁₋₄ alkyl), and —NH (optionally substituted C₁₋₄ alkyl)₂;
R^{w} is absent, or selected from halo, optionally substituted C₁₋₄ alkyl, optionally substituted C₁₋₄ alkoxy, cyano, OH, —NH (optionally substituted C₁₋₄ alkyl), and —NH (optionally substituted C₁₋₄ alkyl)₂;
Rⁿ and R^{z} are each independently hydrogen or optionally substituted C₁₋₆ alkyl, optionally substituted cycloalkyl, or optionally substituted heterocyclyl; and
the dashed line indicates the part of the structure to which the chemical linking moiety (L) is attached.

3. A compound of claim 1 or 2, wherein:
A is 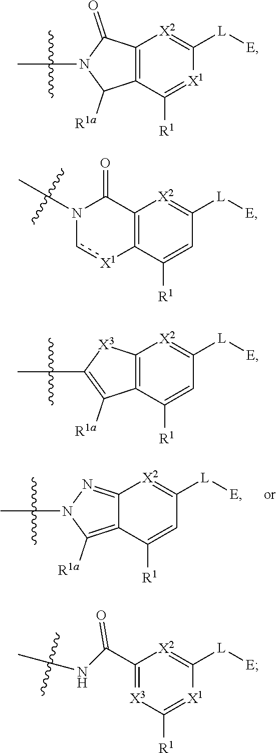
Ring B is phenyl or a 5 to 6-membered monocyclic heteroaryl;
X¹, X², and X³ are each independently CH or N;
X³ is O or S;
each R^{1a} is independently hydrogen or optionally substituted C₁-C₄ alkyl;
R¹ is selected from hydrogen, optionally substituted C₁-C₄ alkyl and optionally substituted (C₃-C₅)cycloalkyl;
R² and R³ are each independently selected from hydrogen, optionally substituted C₁-C₄ alkyl, optionally substituted cycloalkyl, and optionally substituted heterocyclyl; or R² and R³ are taken together to form optionally substituted (C₃-C₄)cycloalkyl or optionally substituted 4-6 membered heterocyclyl;
R⁷ is hydrogen when n is 1, 2, or 3; or R² is hydrogen and R⁷ and R³, together with the atoms to which they are attached, form optionally substituted (C₃-C₄)cycloalkyl when n is 1;
R⁴ is selected from hydrogen, optionally substituted C₁-C₄ alkyl and optionally substituted (C₃-C₆)cycloalkyl;
n is 0, 1, 2, or 3;
L is a chemical linking moiety; and
E is a cereblon E3 ligase-binding moiety represented by the chemical structure: 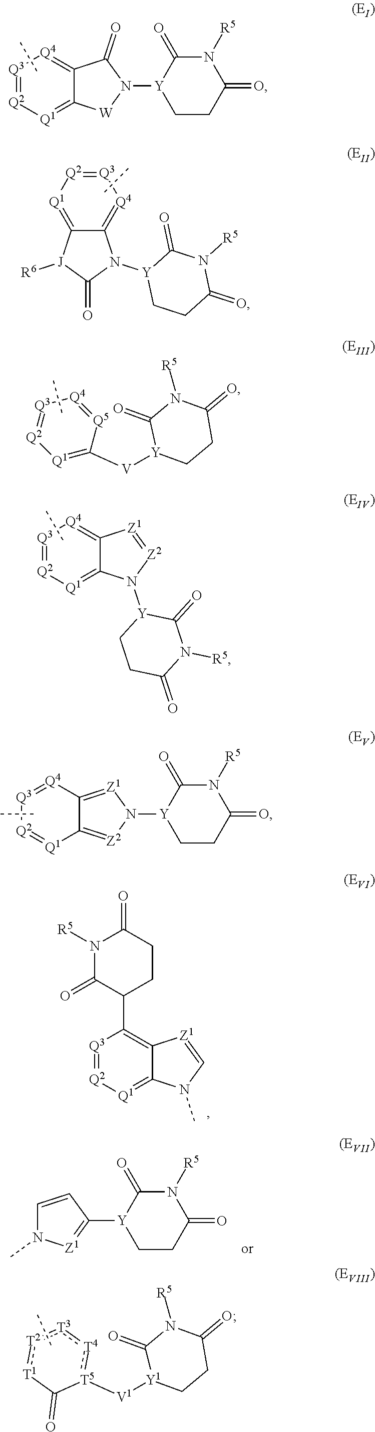
wherein:
W is CH₂, CHR^{v}, SO₂, or C(O);
Y and Y¹ are each independently N, CH, or CR^{y};
Q¹, Q², Q³, Q⁴, Q⁵, T¹, T², T³, T⁴, and T⁵ are each independently CH, CR^{w}, N, or NR^{w};
Z¹ and Z² are each independently CH, CR^{x} or N;
V is absent or is NRⁿ or C(O)NR^{z};
V¹ is absent or C₁₋₄ alkylene;
R⁵ and R⁶ are each independently hydrogen or optionally substituted C₁₋₄ alkyl;
R^{v}, R^{y}, and R^{x} are each independently selected from halo, optionally substituted C₁₋₄ alkyl, optionally substituted C₁₋₄ alkoxy, cyano, OH, —NH (optionally substituted C₁₋₄ alkyl), and —NH (optionally substituted C₁₋₄ alkyl)₂;
R^{w} is absent, or selected from halo, optionally substituted C₁₋₄ alkyl, optionally substituted C₁₋₄ alkoxy, cyano, OH, —NH (optionally substituted C₁₋₄ alkyl), and —NH (optionally substituted C₁₋₄ alkyl)₂;
Rⁿ and R^{z} are each independently hydrogen or optionally substituted C₁₋₆ alkyl, optionally substituted cycloalkyl, or optionally substituted heterocyclyl; and
the dashed line indicates the part of the structure to which the chemical linking moiety (L) is attached.

4. The compound of any one of claims 1 to 3, wherein the compound has the chemical structure II: 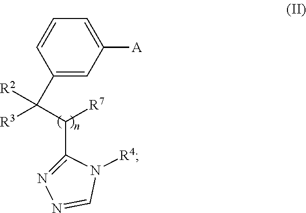
or a pharmaceutically acceptable salt thereof.

5. The compound of any one of claims 1 to 4, or a pharmaceutically acceptable salt thereof, wherein A is

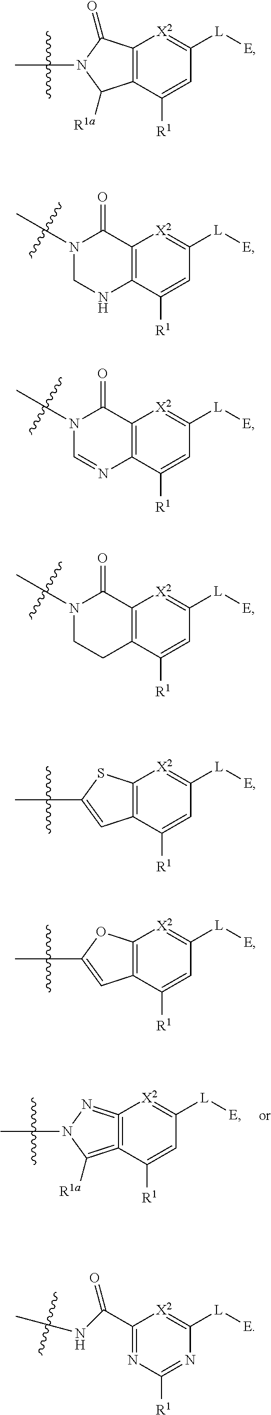

6. The compound of any one of claims 1 to 4, or a pharmaceutically acceptable salt thereof, wherein A is

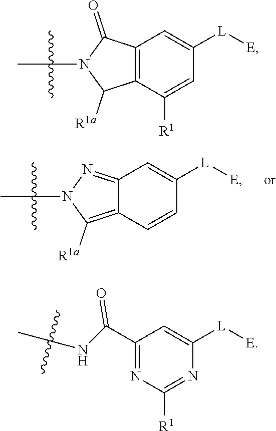

7. The compound of any one of claims 1 to 6, or a pharmaceutically acceptable salt thereof, wherein n is 0 or 1.

8. The compound of any one of claims 1 to 6, or a pharmaceutically acceptable salt thereof, wherein n is 1.

9. The compound of any one of claims 1 to 8, or a pharmaceutically acceptable salt thereof, wherein R^{1a} is hydrogen or C₁-C₄ alkyl.

10. The compound of any one of claims 1 to 9, or a pharmaceutically acceptable salt thereof, wherein R^{1a} is hydrogen.

11. The compound of any one of claims 1 to 10, or a pharmaceutically acceptable salt thereof, wherein R¹ is selected from halo(C₁-C₄ alkyl) and (C₃-C₅)cycloalkyl.

12. The compound of any one of claims 1 to 11, or a pharmaceutically acceptable salt thereof, wherein R¹ is selected from CH₃, CF₃, and cyclopropyl.

13. The compound of any one of claims 1 to 12, or a pharmaceutically acceptable salt thereof, wherein R¹ is CF₃.

14. The compound of any one of claims 1 to 13, or a pharmaceutically acceptable salt thereof, wherein R⁴ is selected from hydrogen, C₁-C₄ alkyl, halo(C₁-C₄ alkyl) and (C₃-C₆)cycloalkyl.

15. The compound of any one of claims 1 to 14, or a pharmaceutically acceptable salt thereof, wherein R⁴ is C₁-C₄ alkyl.

16. The compound of any one of claims 1 to 15, or a pharmaceutically acceptable salt thereof, wherein R⁴ is CH₃.

17. The compound of any one of claims 1 to 16, or a pharmaceutically acceptable salt thereof, wherein R² is halo(C₁-C₄ alkyl), cyano(C₁-C₄ alkyl), hydroxy(C₁-C₄ alkyl), (C₃-C₄)cycloalkyl, or 4- to 6-membered heterocyclyl, wherein said (C₃-C₄)cycloalkyl and 4- to 6-membered heterocyclyl are each optionally substituted with 1 to 3 groups selected from C₁-C₄ alkyl, halo(C₁-C₄ alkyl), cyano(C₁-C₄ alkyl), cyano, halo, C₁-C₄ alkoxy, and halo(C₁-C₄ alkoxy).

18. The compound of any one of claims 1 to 17, or a pharmaceutically acceptable salt thereof, wherein R² is hydroxy(C₁-C₄ alkyl), C₁-C₄ alkyl, cyclobutyl, or tetrahydropyranyl.

19. The compound of any one of claims 1 to 18, or a pharmaceutically acceptable salt thereof, wherein R² is, —CH₃—CH₂OH, cyclobutyl, or tetrahydropyranyl.

20. The compound of any one of claims 1 to 19, or a pharmaceutically acceptable salt thereof, wherein R³ is hydrogen or C₁-C₄ alkyl.

21. The compound of any one of claims 1 to 20, or a pharmaceutically acceptable salt thereof, wherein R³ is hydrogen or CH₃.

22. The compound of any one of claims 1 to 16, or a pharmaceutically acceptable salt thereof, wherein R² and R³ are taken together to form (C₃-C₄)cycloalkyl or 4- to 6-membered heterocyclyl each optionally substituted with 1 to 3 groups selected from C₁-C₄ alkyl, halo(C₁-C₄ alkyl), cyano(C₁-C₄ alkyl), cyano, halo, C₁-C₄ alkoxy, and halo(C₁-C₄ alkoxy).

23. The compound of any one of claims 1 to 16 and 22, or a pharmaceutically acceptable salt thereof, wherein R² and R³ are taken together to form cyclopropyl, cyclobutyl, tetrahydropyranyl, or oxetanyl each optionally substituted with 1 to 3 groups selected from C₁-C₄ alkyl, halo(C₁-C₄ alkyl), cyano(C₁-C₄ alkyl), cyano, halo, C₁-C₄ alkoxy, and halo(C₁-C₄ alkoxy).

24. The compound of any one of claims 1 to 16, 22 or 23, or a pharmaceutically acceptable salt thereof, wherein R² and R³ are taken together to form cyclopropyl, cyclobutyl, tetrahydropyranyl, or oxetanyl each optionally substituted with 1 to 3 groups selected from C₁-C₄ alkyl, halo(C₁-C₄ alkyl), cyano(C₁-C₄ alkyl) and cyano.

25. The compound of any one of claims 1 to 16, and 22 to 24, or a pharmaceutically acceptable salt thereof, wherein R² and R³ are taken together to form cyclopropyl, cyclobutyl, or oxetanyl each optionally substituted with CH₃ or cyano.

26. The compound of any one of claims 1 to 25, or a pharmaceutically acceptable salt thereof, wherein E is represented by the chemical structure:

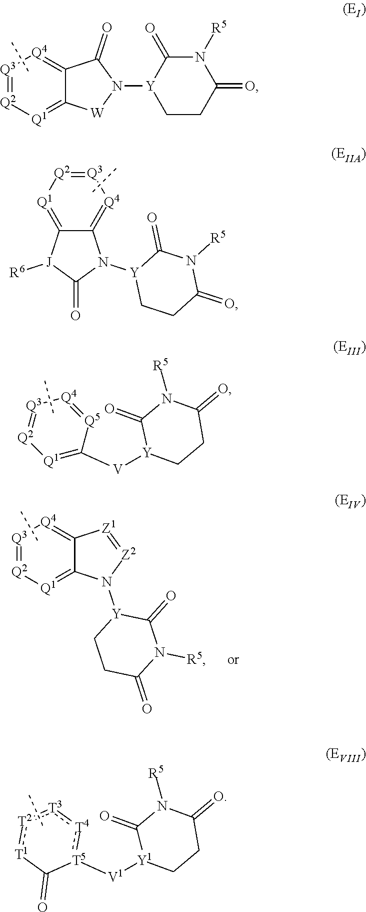

27. The compound of any one of claims 1 to 25, or a pharmaceutically acceptable salt thereof, wherein E is represented by the chemical structure:

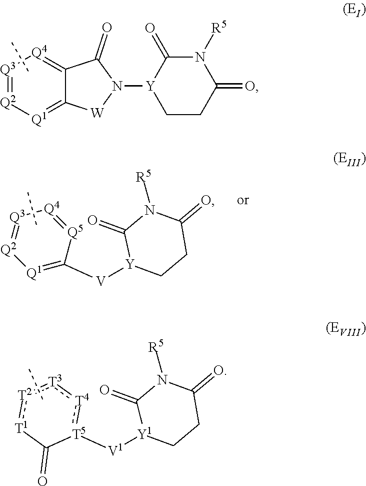

28. The compound of any one of claims 1 to 26, or a pharmaceutically acceptable salt thereof, wherein E is represented by the chemical structure:

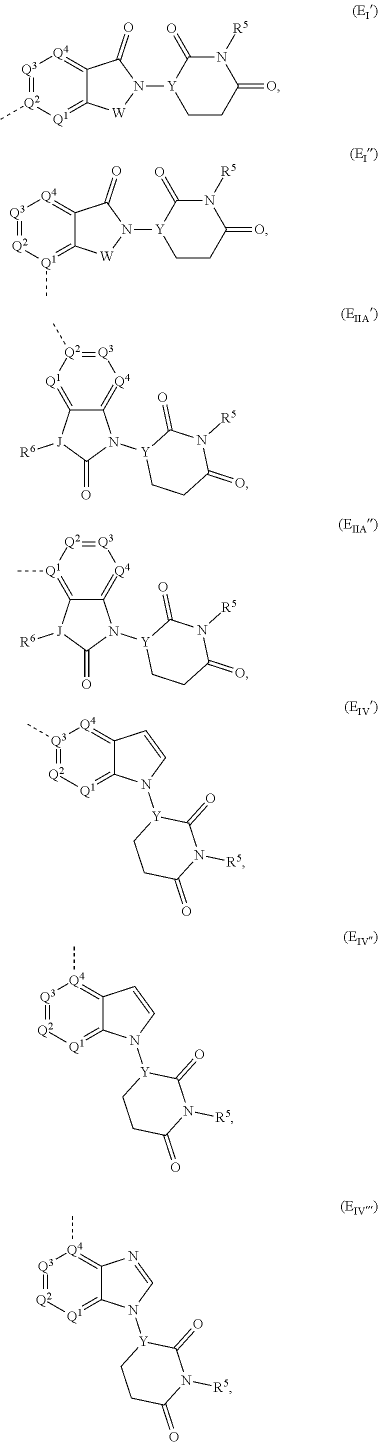

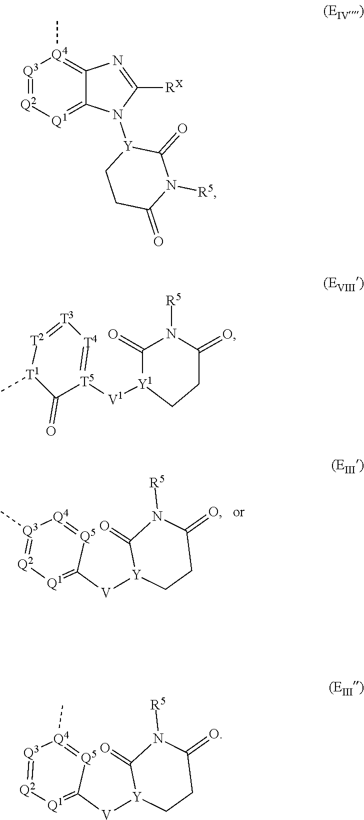

29. The compound of any one of claims 1 to 26, or a pharmaceutically acceptable salt thereof, wherein E is represented by the chemical structure:

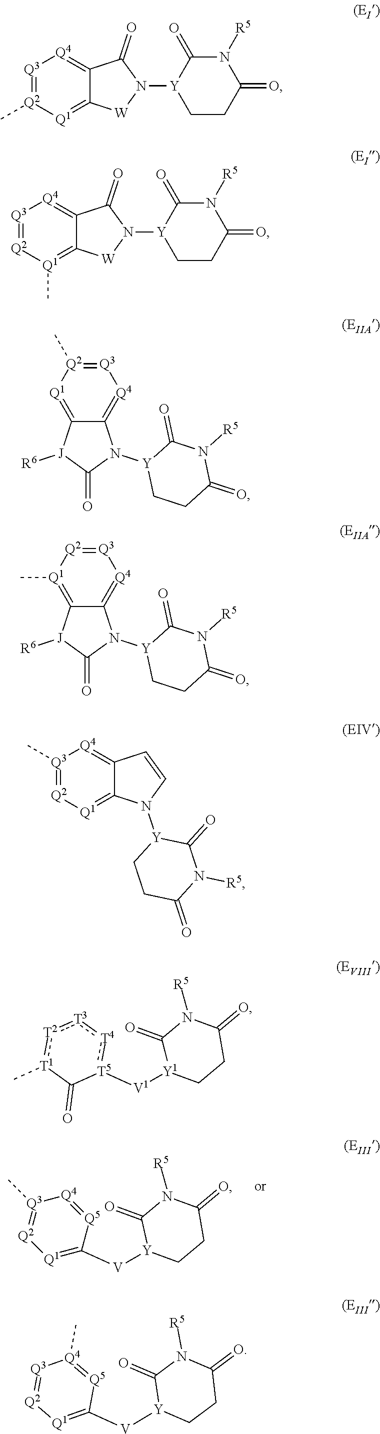

30. The compound of any one of claims 1 to 27, or a pharmaceutically acceptable salt thereof, wherein E is represented by the chemical structure:

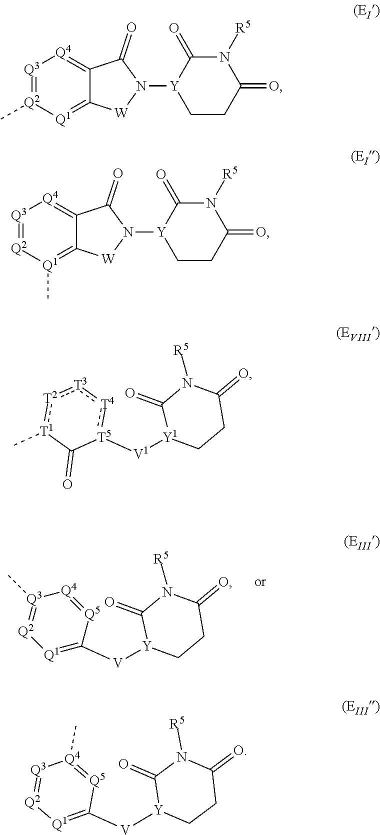

31. The compound of any one of claims 1 to 30, or a pharmaceutically acceptable salt thereof, wherein Y is CH.

32. The compound of any one of claims 1 to 30, or a pharmaceutically acceptable salt thereof, wherein Y is N.

33. The compound of any one of claims 1 to 32, or a pharmaceutically acceptable salt thereof, wherein R⁵ is hydrogen.

34. The compound of any one of claim 1 to 33, or a pharmaceutically acceptable salt thereof, wherein J is N.

35. The compound of any one of claims 1 to 34, or a pharmaceutically acceptable salt thereof, wherein R⁶ is H, C₁₋₄ alkyl, C₁₋₄ haloalkyl, or C₃₋₄cycloalkyl.

36. The compound of any one of claims 1 to 34, or a pharmaceutically acceptable salt thereof, wherein R⁶ is H or C₁₋₄ alkyl.

37. The compound of any one of claims 1 to 34, or a pharmaceutically acceptable salt thereof, wherein R⁶ is H, —CH₃, —CH(CH₃)₂, —CHF₂, or cyclopropyl.

38. The compound of any one of claims 1 to 34, or a pharmaceutically acceptable salt thereof, wherein R⁶ is H, —CH₃, or —CH(CH₃)₂.

39. The compound of any one of claims 1 to 38, or a pharmaceutically acceptable salt thereof, wherein W is CH₂, —CH(C₁₋₄ alkyl), or C(O).

40. The compound of any one of claims 1 to 39, or a pharmaceutically acceptable salt thereof, wherein V is absent or C(O)NH.

41. The compound of any one of claims 1 to 40, or a pharmaceutically acceptable salt thereof, wherein V¹ is CH₂.

42. The compound of any one of claims 1 to 41, or a pharmaceutically acceptable salt thereof, wherein Yⁱ is CH.

43. The compound of any one of claims 1 to 42, or a pharmaceutically acceptable salt thereof, wherein Yⁱ is N.

44. The compound of any one of claims 1 to 43, or a pharmaceutically acceptable salt thereof, wherein T¹ is N.

45. The compound of any one of claims 1 to 44, or a pharmaceutically acceptable salt thereof, wherein T², T³, and T⁴ are each CH.

46. The compound of any one of claims 1 to 41, or a pharmaceutically acceptable salt thereof, wherein Q¹ for the chemical structure E₁, E₁′, or E₁″ is N, CR^{w}, or CH.

47. The compound of any one of claims 1 to 45, or a pharmaceutically acceptable salt thereof, wherein Q¹, Q², Q³, and Q⁴ for the chemical structure E_{I}, E_{I}′, E_{I}″, E_{IIA}′, or E_{IV}′ are each independently CH or CR^{w}.

48. The compound of any one of claims 1 to 45, or a pharmaceutically acceptable salt thereof, wherein Q², Q³, and Q⁴ for the chemical structure E₁, E₁′, or E₁″ are each independently CH or CR^{w}.

49. The compound of any one of claims 1 to 48, or a pharmaceutically acceptable salt thereof, wherein Q⁵ for the chemical structure E_{III}, E_{III}′, or E_{III}″ is CH or CR^{w}.

50. The compound of any one of claims 1 to 49, or a pharmaceutically acceptable salt thereof, wherein Q¹, Q², Q³, Q⁴, and Q⁵ for the chemical structure E_{III}, E_{III}′, or E_{III}″ are each CH, N, or CR^{w}.

51. The compound of any one of claims 1 to 49, or a pharmaceutically acceptable salt thereof, wherein Q², Q³, Q⁴, and Q⁵ for the chemical structure E_{III}, E_{III}′, or E_{III}″ are each CH or CR^{w}.

52. The compound of any one of claims 1 to 51, or a pharmaceutically acceptable salt thereof, wherein R^{w} is absent or is halo, C₁-C₄ alkoxy, or C₁-C₄ alkyl.

53. The compound of any one of claims 1 to 52, or a pharmaceutically acceptable salt thereof, wherein R^{w} is absent or is fluoro, chloro, OCH₃, or CH₃.

54. The compound of any one of claims 1 to 53, or a pharmaceutically acceptable salt thereof, wherein R^{x} is C₁₋₄alkoxy.

55. The compound of any one of claims 1 to 53, or a pharmaceutically acceptable salt thereof, wherein R^{x} is —OCH₃.

56. The compound of any one of claims 1 to 25, or a pharmaceutically acceptable salt thereof, wherein E is selected from:

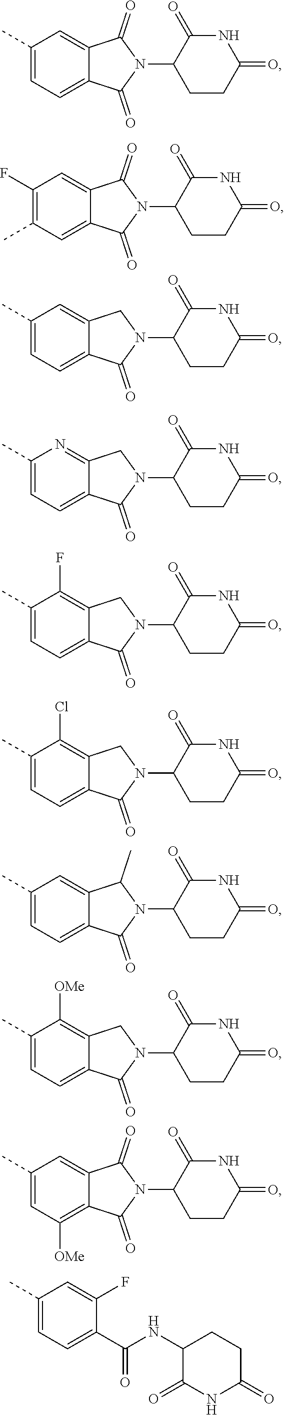

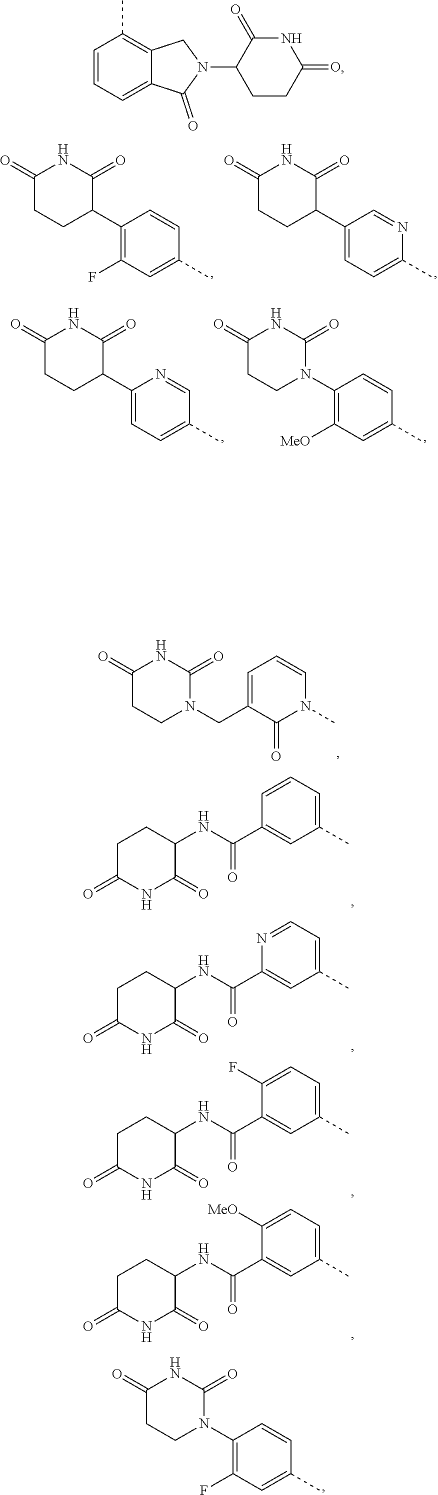

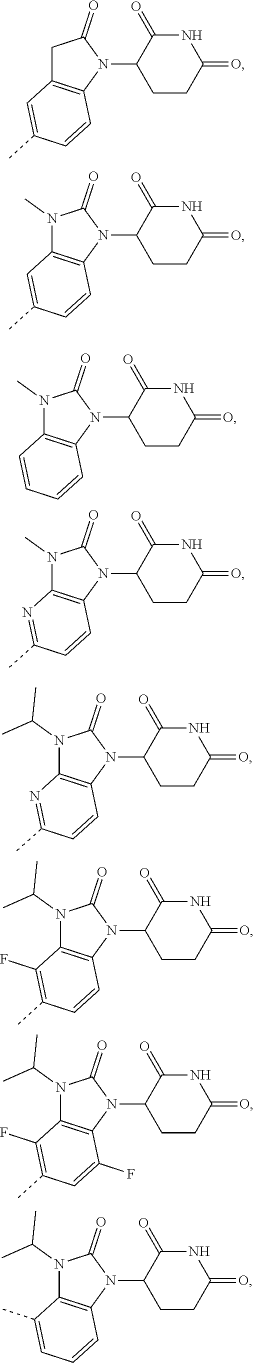

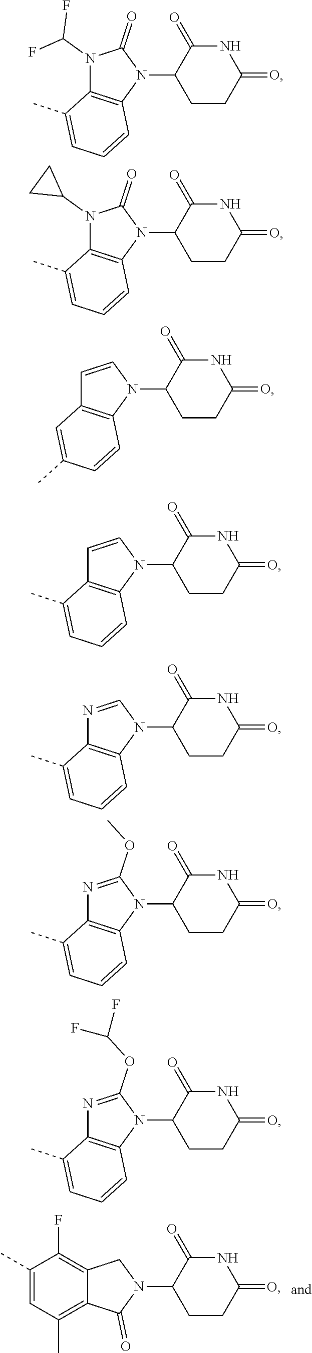

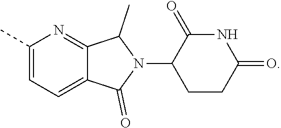

57. The compound of any one of claims 1 to 25, or a pharmaceutically acceptable salt thereof, wherein E is selected from:

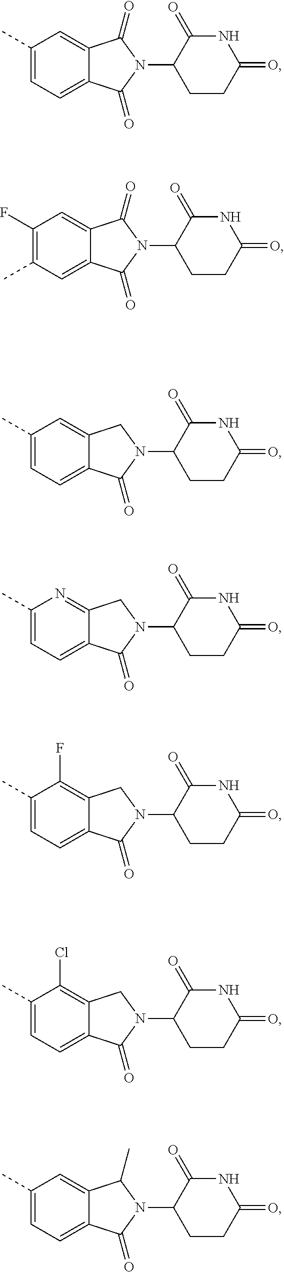

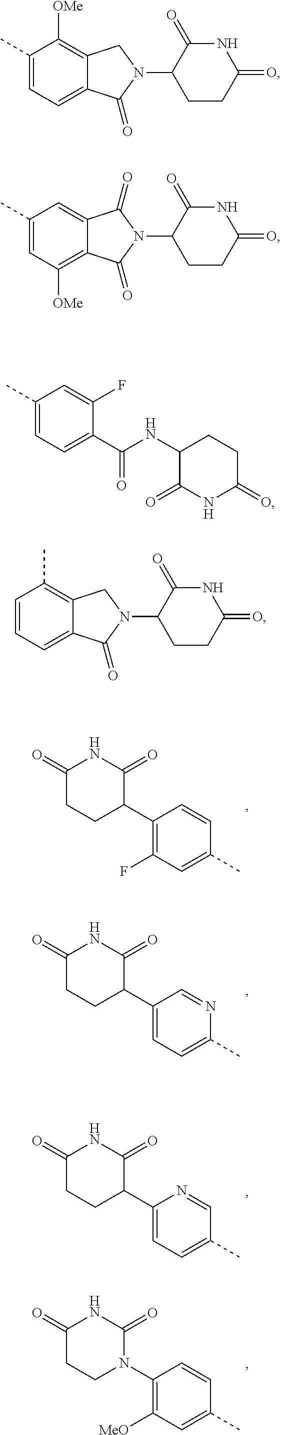

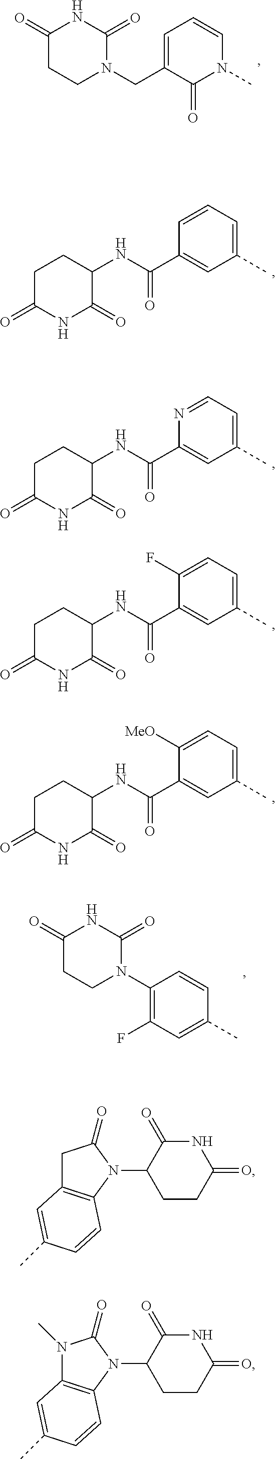

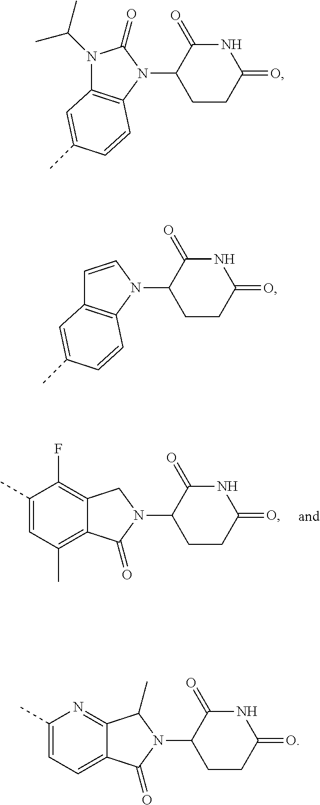

58. The compound of any one of claims 1 to 25, or a pharmaceutically acceptable salt thereof, wherein E is selected from:

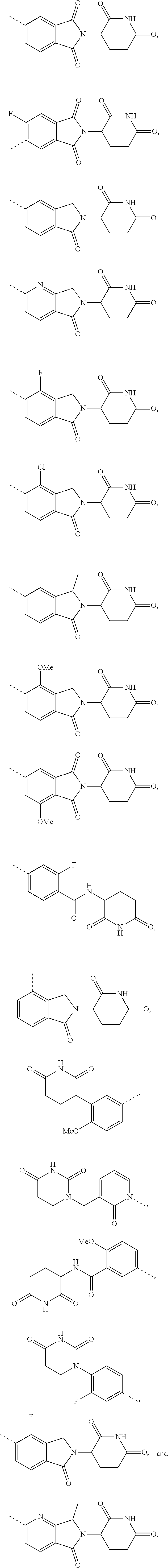

59. The compound according to any one of claims 1 to 58, or a pharmaceutically acceptable salt thereof, wherein the chemical linking moiety (L) is represented by the chemical structure: 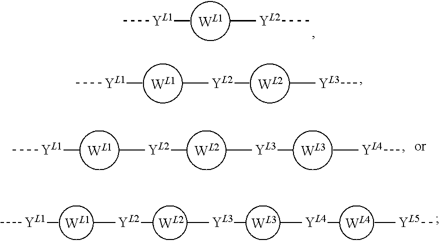
wherein:
Y^{L1}, Y^{L2}, Y^{L3}, Y^{L4} and Y^{L5} are each independently absent or selected from O, NR^{Y}, S, SO, SO₂, SO₂NR^{Y}, C(O), C(O)O, C(O)NR^{Y}, and an optionally substituted C₁₋₆ alkylene, wherein said C₁₋₆ alkylene may also be optionally interrupted by one or more O, NH, and N(C₁₋₄ alkyl), and wherein two hydrogens on the same carbon of said C₁₋₆ alkylene may be taken together to form oxo;
R^{Y} is H or C₁₋₄ alkyl; and
W^{L1}, W^{L2}, W^{L3}, and W^{L4} are each independently selected from phenyl, heterocyclyl, heteroaryl, and cycloalkyl, each of which are optionally substituted.

60. The compound according to claim 59, or a pharmaceutically acceptable salt thereof, wherein
Y^{L1}, Y^{L2}, Y^{L3}, Y^{L4} and Y^{L5} are each independently absent or selected from O, NH, N(C₁₋₄ alkyl), and a C₁₋₆ alkylene optionally interrupted by one or more O, NH, and N(C₁₋₄ alkyl), and wherein two hydrogens on the same carbon of said C₁₋₆ alkylene may be taken together to form oxo;
W^{L1}, W^{L2}, W^{L3}, and W^{L4} are each independently selected from phenyl, heterocyclyl, heteroaryl, and cycloalkyl, each of which are optionally substituted with 1 or 2 C₁₋₄ alkyl, or two substituents together form C₃₋₄cycloalkyl or 4- to 6-membered heterocyclyl.

61. The compound according to claim 59 or 60, or a pharmaceutically acceptable salt thereof, wherein
Y^{L1}, Y^{L2}, Y^{L3}, Y^{L4} and Y^{L5} are each independently absent or selected from O, NH, N(C₁₋₄ alkyl), and a C₁₋₆ alkylene optionally interrupted by one or more O, NH, and N(C₁₋₄ alkyl), and wherein two hydrogens on the same carbon of said C₁₋₆ alkylene may be taken together to form oxo;
W^{L1}, W^{L2}, W^{L3}, and W^{L4} are each independently selected from heterocyclyl, heteroaryl, and cycloalkyl, each of which are optionally substituted with 1 or 2 C₁₋₄ alkyl, or two substituents together form C₃₋₄cycloalkyl.

62. The compound according to any one of claims 59 to 61, or a pharmaceutically acceptable salt thereof, wherein
Y^{L1}, Y^{L2}, Y^{L3}, Y^{L4} and Y^{L5} are each independently absent or selected from O, NH, N(C₁₋₄ alkyl), and a C₁₋₆ alkylene optionally interrupted by one or more O, NH, and N(C₁₋₄ alkyl), and wherein two hydrogens on the same carbon of said C₁₋₆ alkylene may be taken together to form oxo;
W^{L1}, W^{L2}, W^{L3}, and W^{L4} are each independently selected from heterocyclyl, heteroaryl, and cycloalkyl, each of which are optionally substituted with 1 or 2 C₁₋₄ alkyl.

63. The compound according to any one of claims 59 to 62, or a pharmaceutically acceptable salt thereof, wherein Y^{L1} is absent or C₁₋₆ alkylene optionally interrupted by one or more O, NH, and N(C₁₋₄ alkyl).

64. The compound according to any one of claims 59, 60, or 63, or a pharmaceutically acceptable salt thereof, wherein W^{L1} is selected from phenyl, 4- to 11-membered heterocyclyl, and 3- to 6-membered cycloalkyl, each of which is optionally substituted with 1 or 2 C₁₋₄ alkyl, or two substituents together form C₃₋₄cycloalkyl or oxetanyl.

65. The compound according to any one of claims 59 to 61 or 63, or a pharmaceutically acceptable salt thereof, wherein W^{L1} is selected from 4- to 11-membered heterocyclyl and 3-to 6-membered cycloalkyl, each of which is optionally substituted with 1 or 2 C₁₋₄ alkyl, or two substituents together form C₃₋₄cycloalkyl.

66. The compound according to any one of claims 59 to 63, or a pharmaceutically acceptable salt thereof, wherein W^{L1} is selected from 4- to 11-membered heterocyclyl and 3-to 6-membered cycloalkyl, each of which is optionally substituted with 1 or 2 C₁₋₄ alkyl.

67. The compound according to any one of claims 59, 60, 63, or 64, or a pharmaceutically acceptable salt thereof, wherein W^{L1} is selected from cyclobutyl, cyclohexyl, azetidinyl, pyrrolidinyl, piperidinyl, piperazinyl, 1,4-diazepanyl, 2,5-diazabicyclo[2.2.1]heptanyl, 2,6-diazaspiro[3.4]octanyl, 2,7-diazaspiro[4.4]nonanyl, 3,9-diazaspiro[5.5]undecanyl, 2,7-diazaspiro[3.5]nonanyl, 2,6-diazaspiro[3.3]heptanyl, 2,6-diazaspiro[3.4]octanyl, and 2-azaspiro[3.3]heptanyl each of which are optionally substituted with 1 or 2 C₁₋₄ alkyl, or two substituents together form C₃₋₄cycloalkyl or oxetanyl.

68. The compound according to any one of claims 59 to 61 and 63, or a pharmaceutically acceptable salt thereof, wherein W^{L1} is selected from cyclobutyl, cyclohexyl, azetidinyl, pyrrolidinyl, piperidinyl, piperazinyl, 1,4-diazepanyl, 2,5-diazabicyclo[2.2.1]heptanyl, 2,6-diazaspiro[3.4]octanyl, 2,7-diazaspiro[4.4]nonanyl, 3,9-diazaspiro[5.5]undecanyl, 2,7-diazaspiro[3.5]nonanyl, 2,6-diazaspiro[3.3]heptanyl, 2,6-diazaspiro[3.4]octanyl, and 2-azaspiro[3.3]heptanyl each of which are optionally substituted with 1 or 2 C₁₋₄ alkyl, or two substituents together form C₃₋₄cycloalkyl.

69. The compound according to any one of claims 59 to 63, or a pharmaceutically acceptable salt thereof, wherein W^{L1} is selected from cyclobutyl, cyclohexyl, azetidinyl, pyrrolidinyl, piperidinyl, piperazinyl, 3,9-diazaspiro[5.5]undecanyl, 2,7-diazaspiro[3.5]nonanyl, 2,6-diazaspiro[3.3]heptanyl, 2,6-diazaspiro[3.4]octanyl, and 2-azaspiro[3.3]heptanyl each of which are optionally substituted with 1 or 2 C₁₋₄ alkyl.

70. The compound according to any one of claims 59 to 69, or a pharmaceutically acceptable salt thereof, wherein Y^{L2} is absent or selected from O and C₁₋₆ alkylene optionally interrupted by one or more O, NH, and N(C₁₋₄ alkyl), and wherein two hydrogens on the same carbon of said C₁₋₆ alkylene may be taken together to form oxo.

71. The compound according to any one of claims 59 to 70, or a pharmaceutically acceptable salt thereof, wherein W^{L2} is selected from 5- to 6-membered heteroaryl, 4- to 11-membered heterocyclyl and 3- to 6-membered cycloalkyl, each of which is optionally substituted with 1 or 2 C₁₋₄ alkyl.

72. The compound according to any one of claims 59 to 70, or a pharmaceutically acceptable salt thereof, wherein W^{L2} is selected from cyclobutyl, cyclohexyl, azetidinyl, piperidinyl, piperazinyl, 3,9-diazaspiro[5.5]undecanyl, 2,7-diazaspiro[3.5]nonanyl, 2,6-diazaspiro[3.3]heptanyl, 3-azaspiro[5.5]undecayl, 2-azaspiro[3.3]heptanyl, and pyrimidinyl, each of which are optionally substituted with 1 or 2 C₁₋₄ alkyl.

73. The compound according to any one of claims 59 to 72, or a pharmaceutically acceptable salt thereof, wherein Y^{L3} is absent or selected from O and a C₁₋₆ alkylene, wherein said C₁₋₆ alkylene may be optionally interrupted by O, NH, and N(C₁₋₄ alkyl).

74. The compound according to any one of claims 59 to 73, or a pharmaceutically acceptable salt thereof, wherein W^{L3} is selected from 4- to 11-membered heterocyclyl and 3-to 6-membered cycloalkyl, each of which is optionally substituted with 1 or 2 C₁₋₄ alkyl.

75. The compound according to any one of claims 59 to 73, or a pharmaceutically acceptable salt thereof, wherein W^{L3} is selected from cyclohexyl, azetidinyl, piperidinyl, piperazinyl, and 2,7-diazaspiro[3.5]nonanyl, each of which are optionally substituted with 1 or 2 C₁₋₄ alkyl.

76. The compound according to any one of claims 59 to 74, or a pharmaceutically acceptable salt thereof, wherein Y^{L4} is absent or selected from O and a C₁₋₆ alkylene.

77. The compound according to any one of claims 59 to 76, or a pharmaceutically acceptable salt thereof, wherein W^{L4} is 4- to 7-membered heterocyclyl optionally substituted with 1 or 2 C₁₋₄ alkyl.

78. The compound according to any one of claims 59 to 76, or a pharmaceutically acceptable salt thereof, wherein W^{L4} is selected from piperidinyl and piperazinyl, each of which are optionally substituted with 1 or 2 C₁₋₄ alkyl.

79. The compound according to any one of claims 59 to 78, or a pharmaceutically acceptable salt thereof, wherein Y^{L5} is absent.

80. The compound of any one of claims 1 to 58, or a pharmaceutically acceptable salt thereof, wherein L is selected from 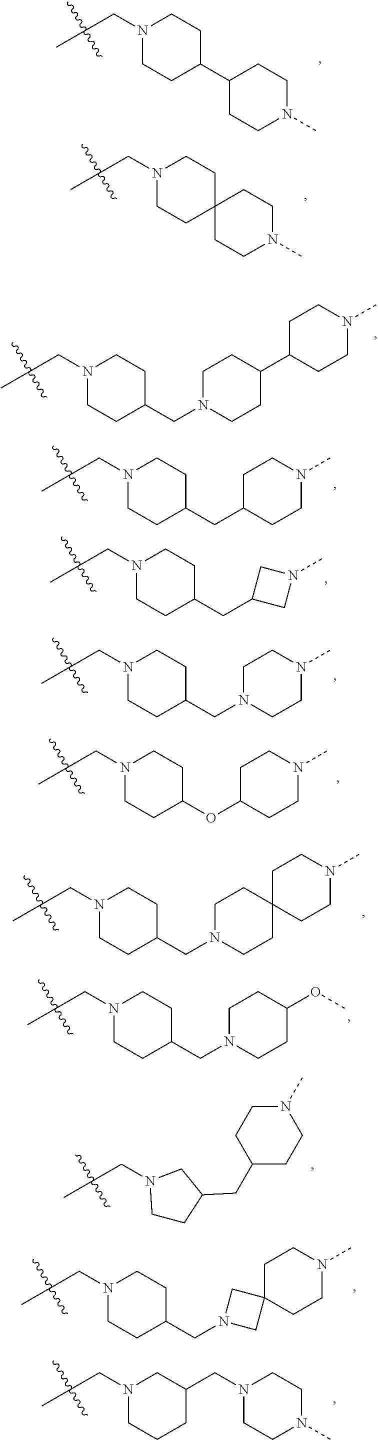 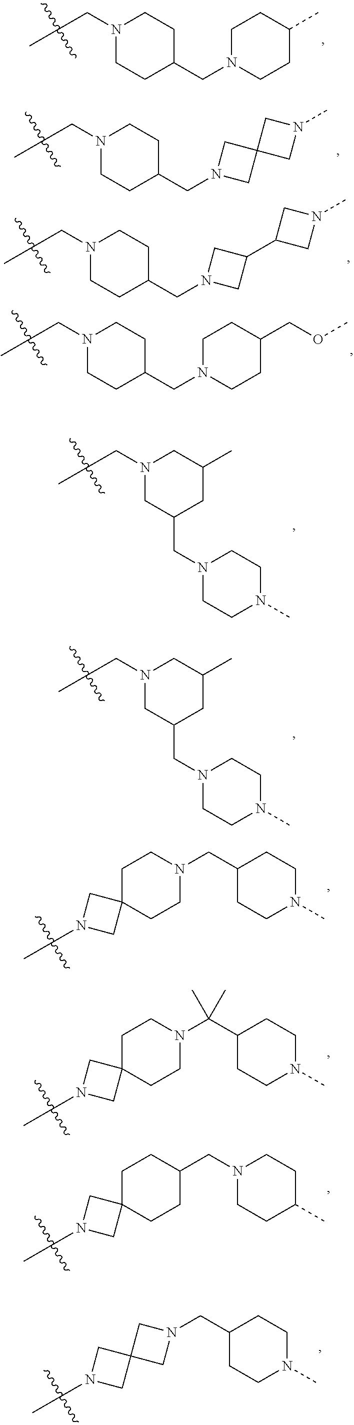 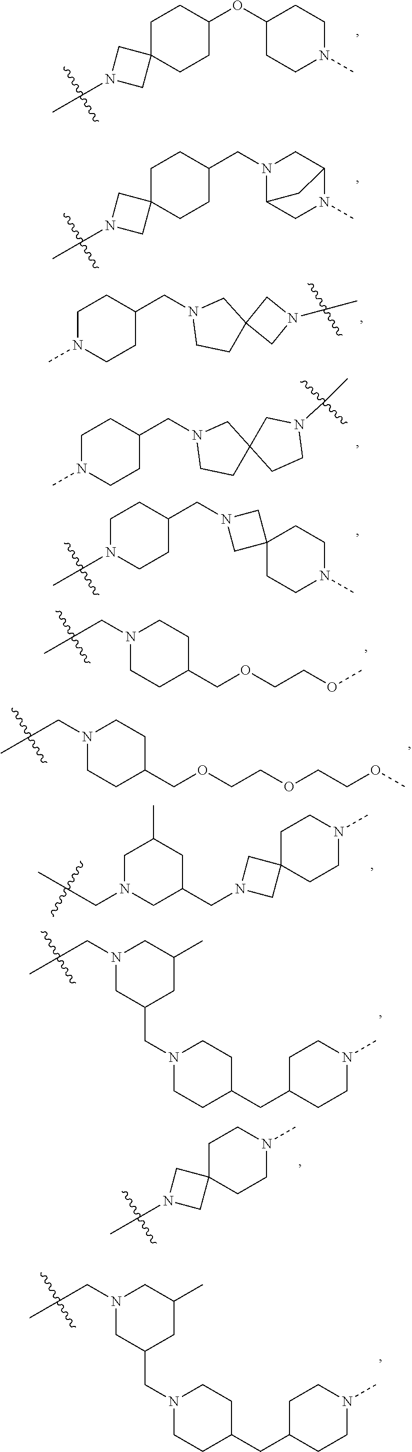 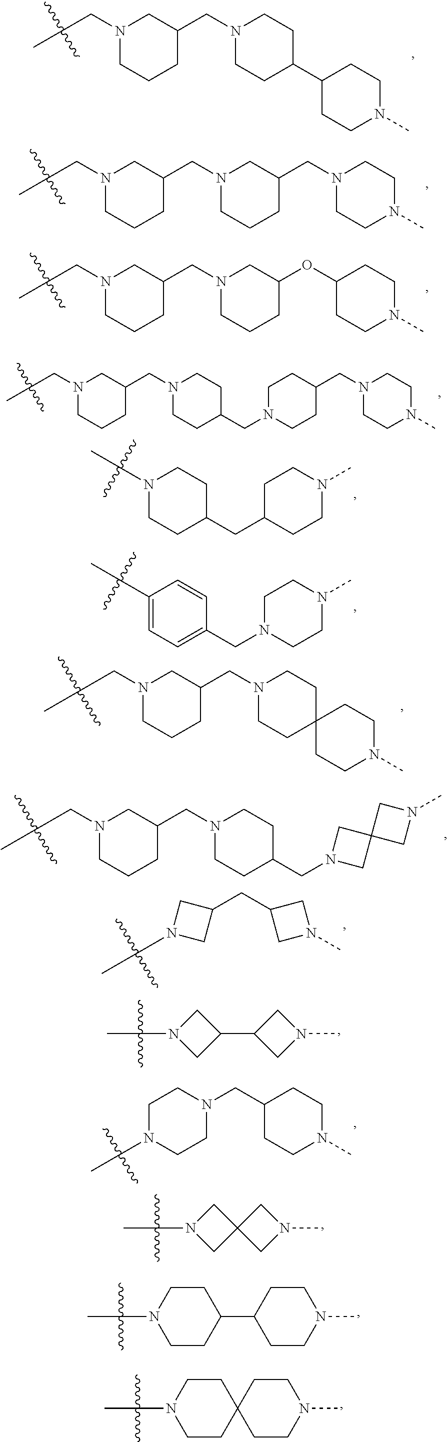 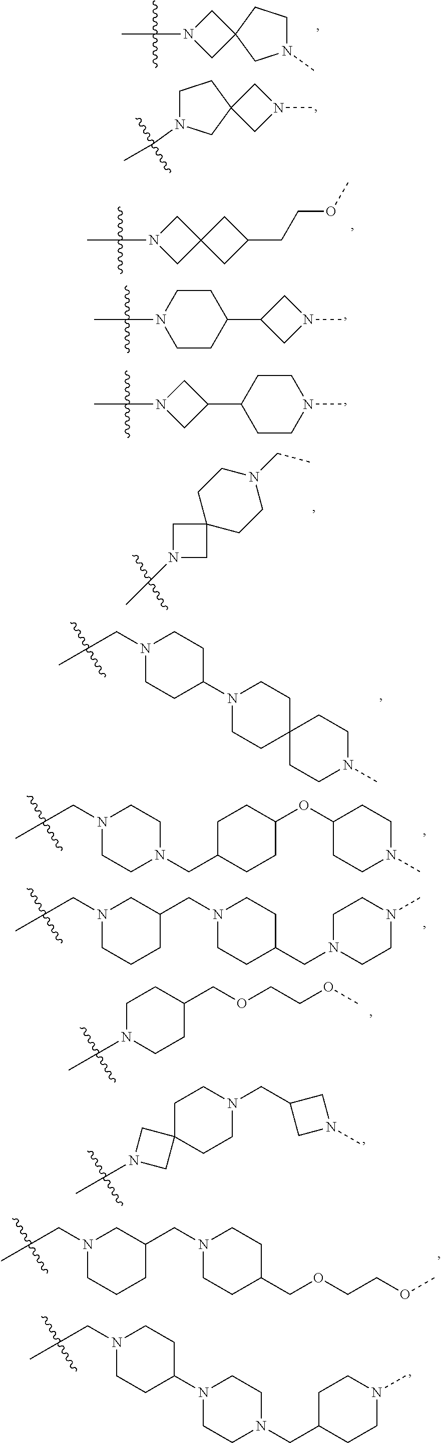 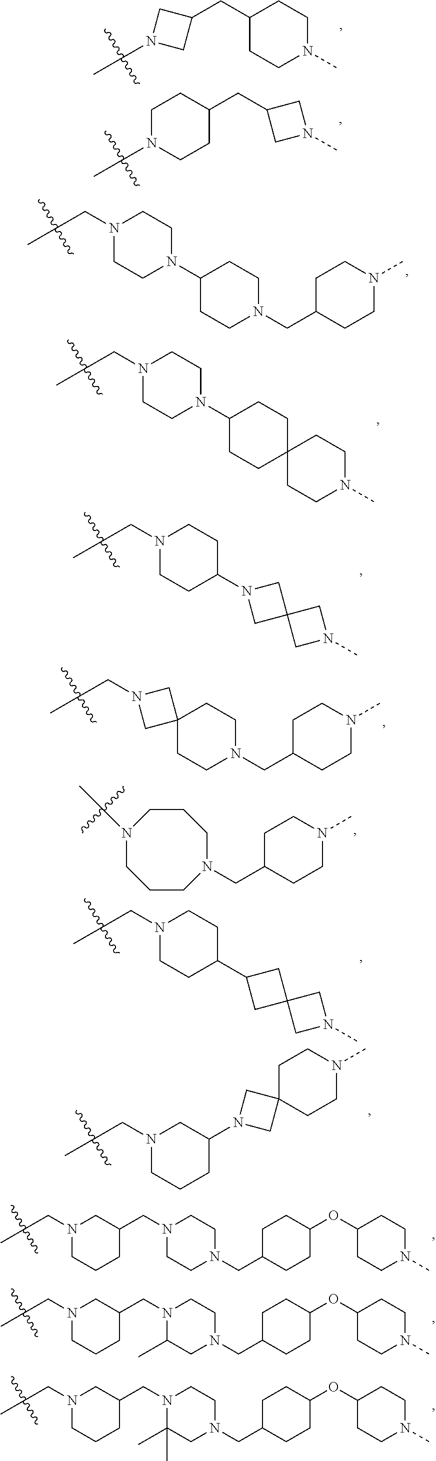 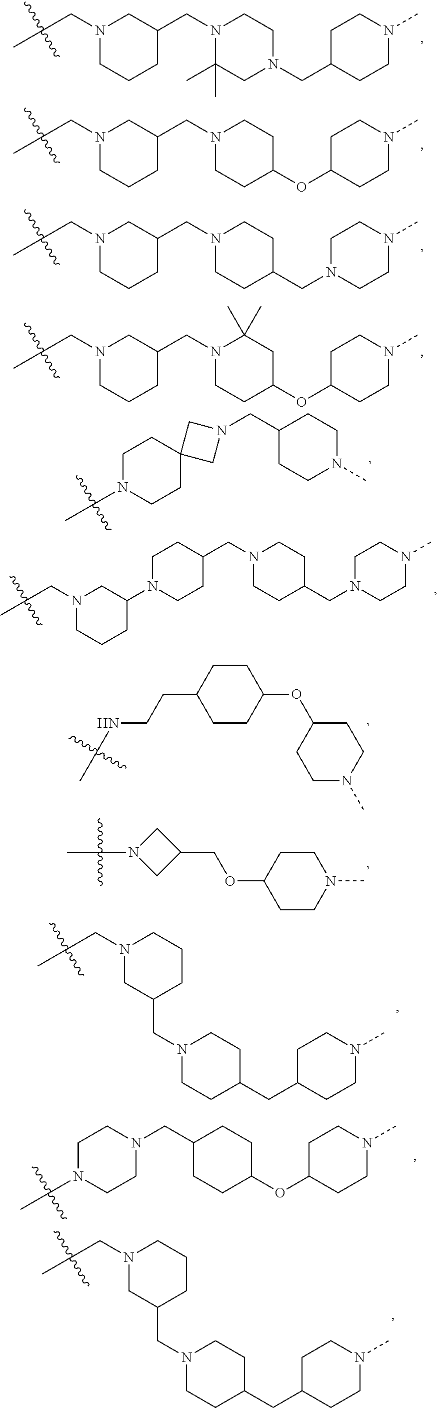 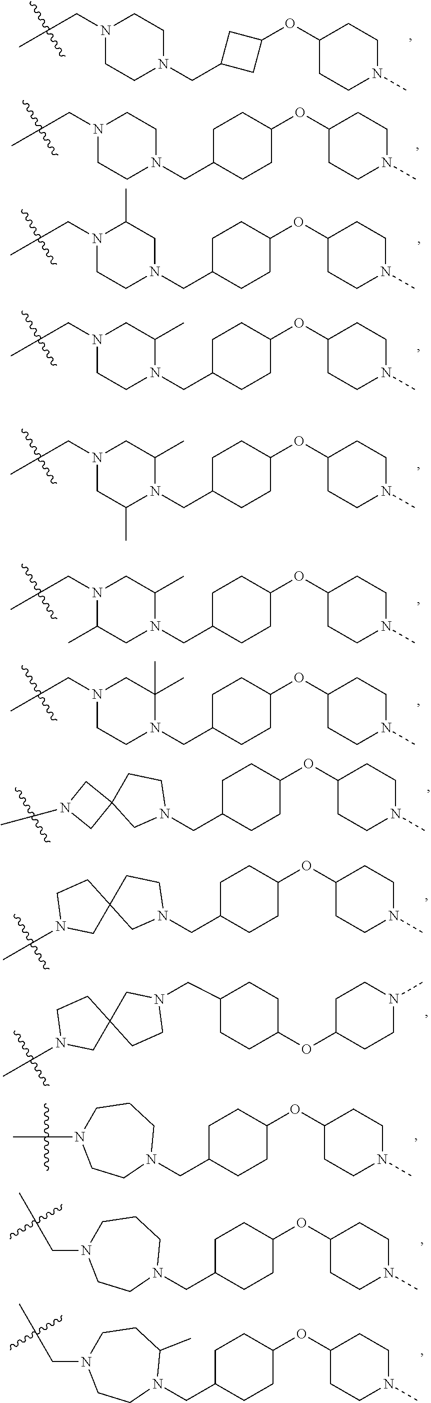 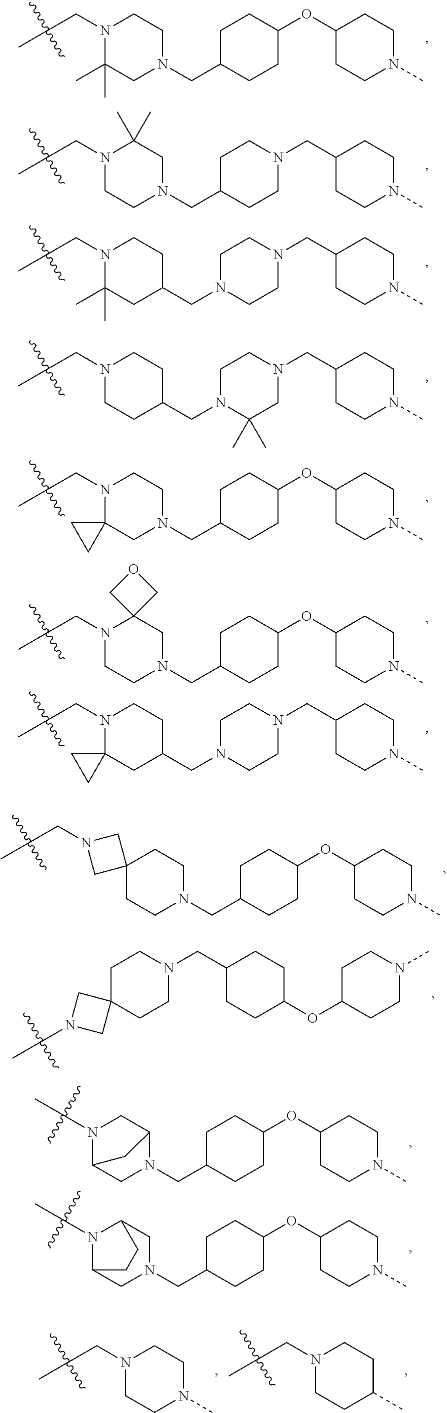 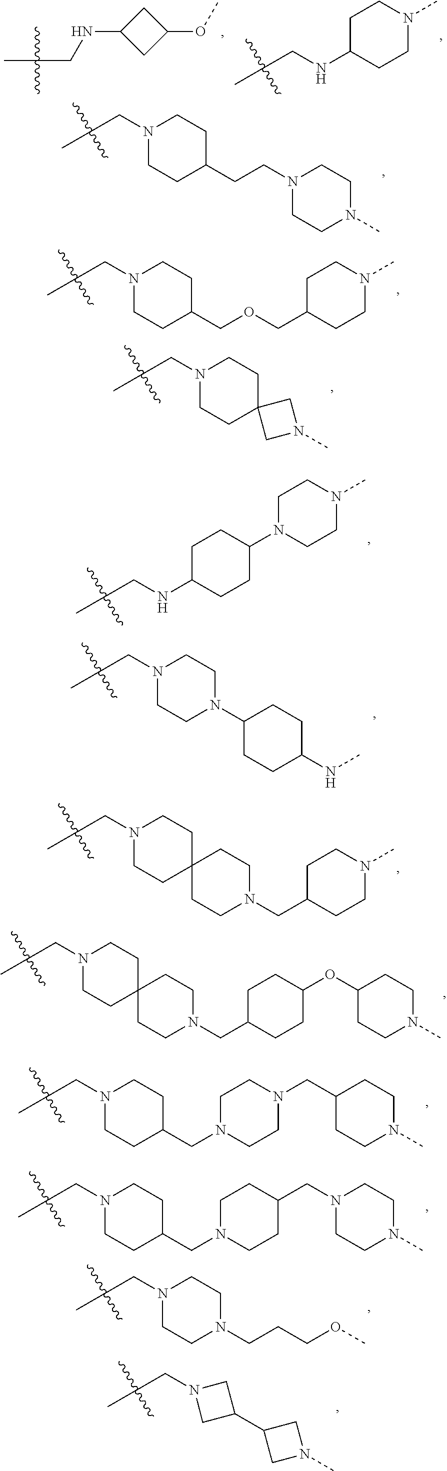 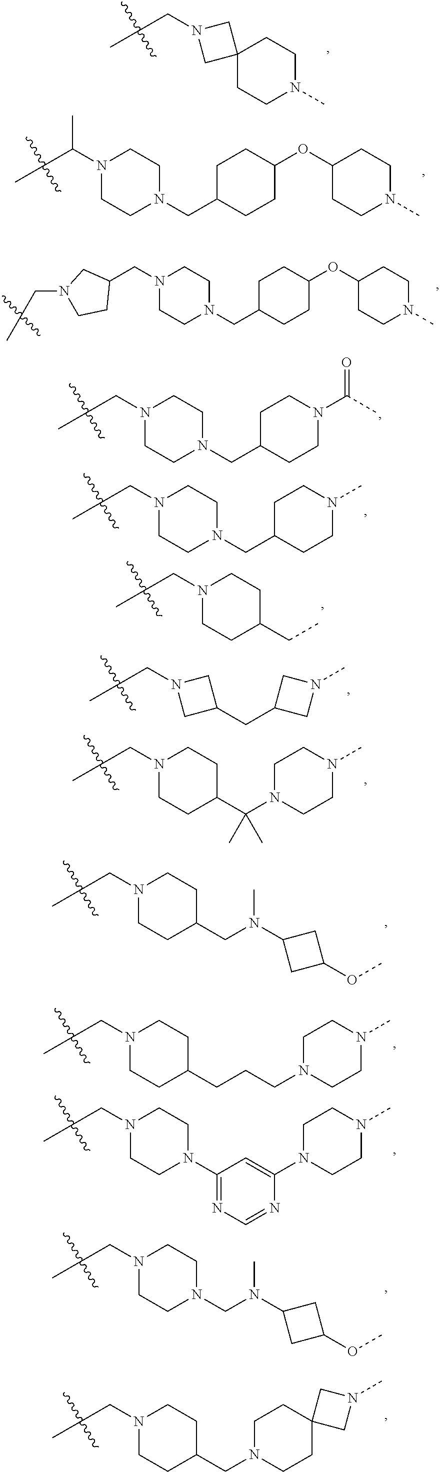 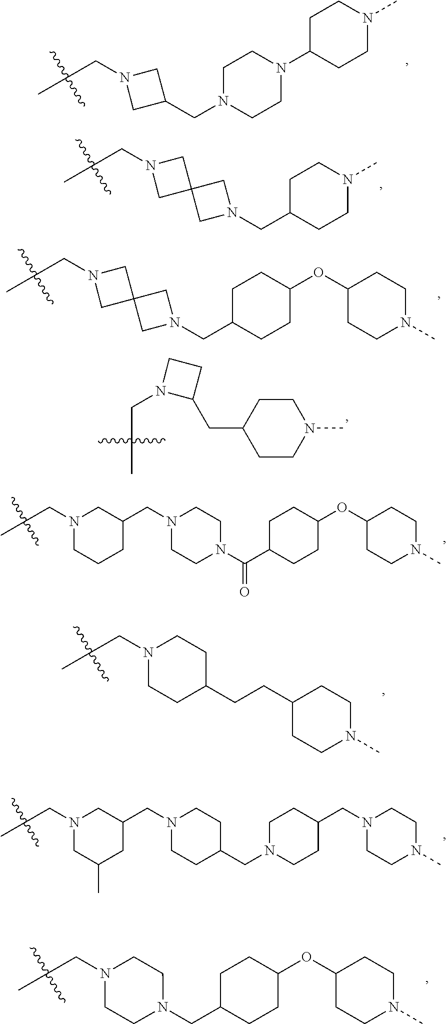 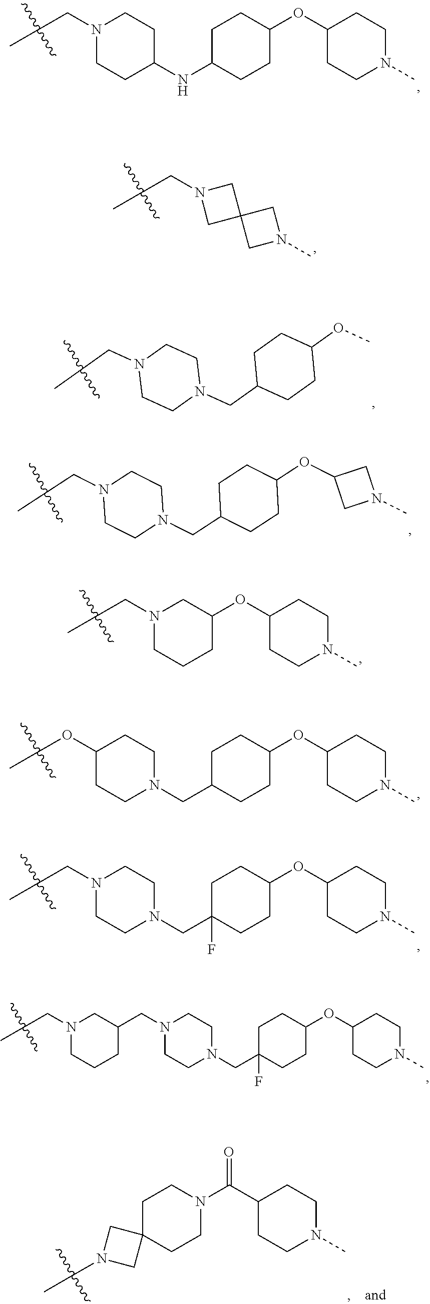 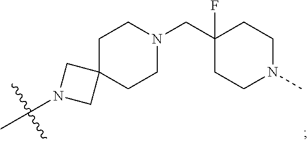
wherein the dashed line indicates the attachment point to E.

81. The compound of any one of claims 1 to 58, or a pharmaceutically acceptable salt thereof, wherein L is selected from 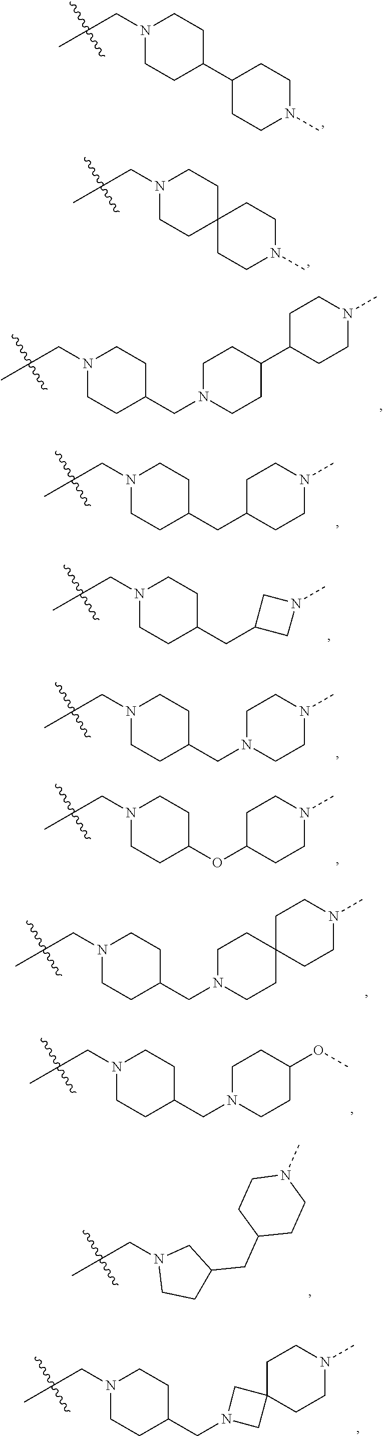 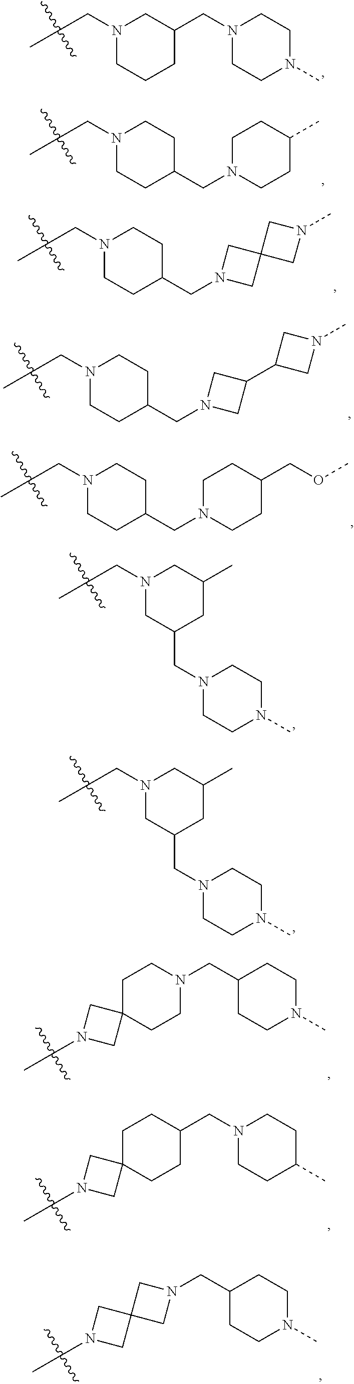 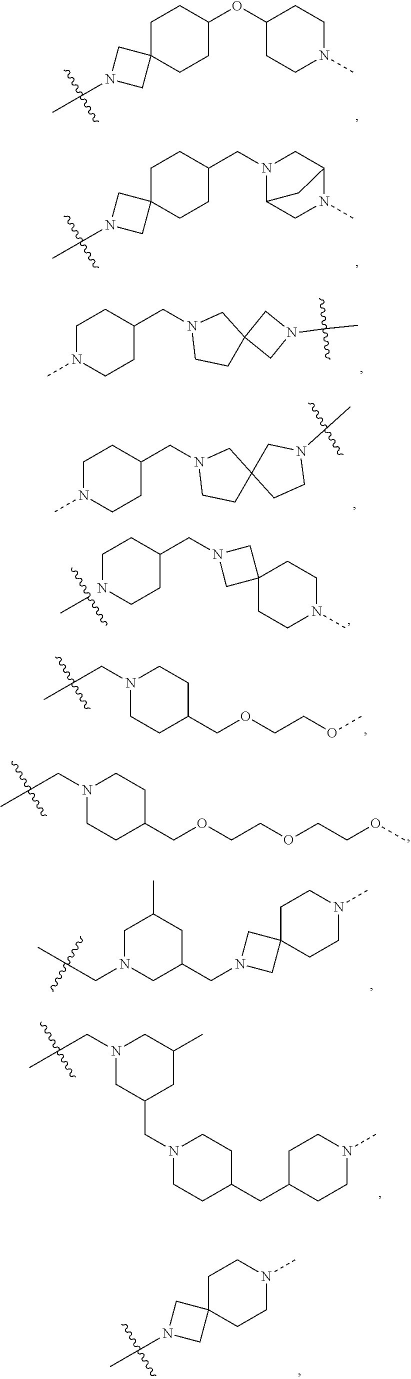 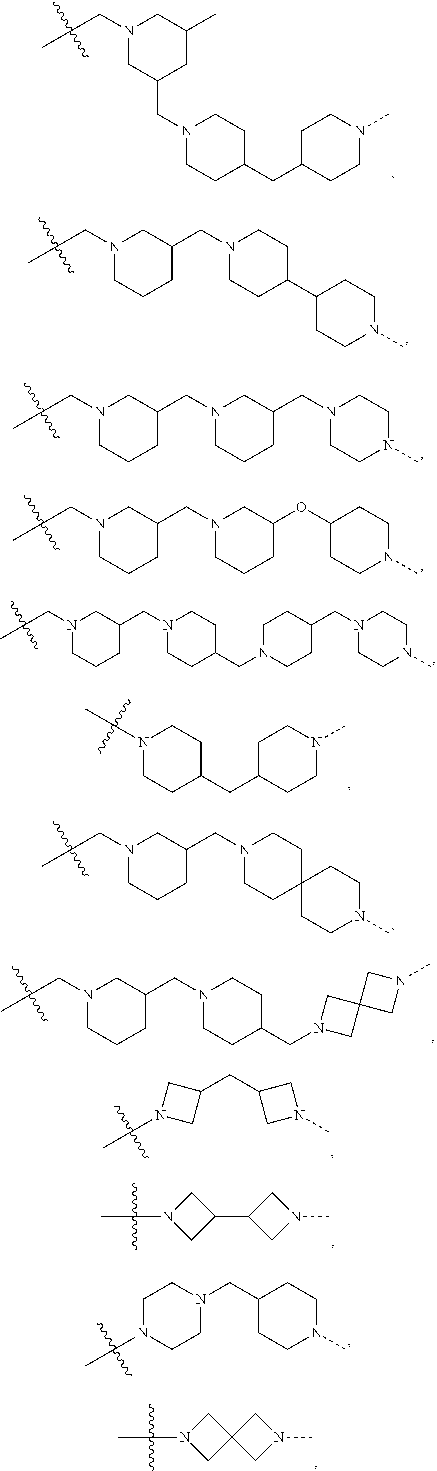 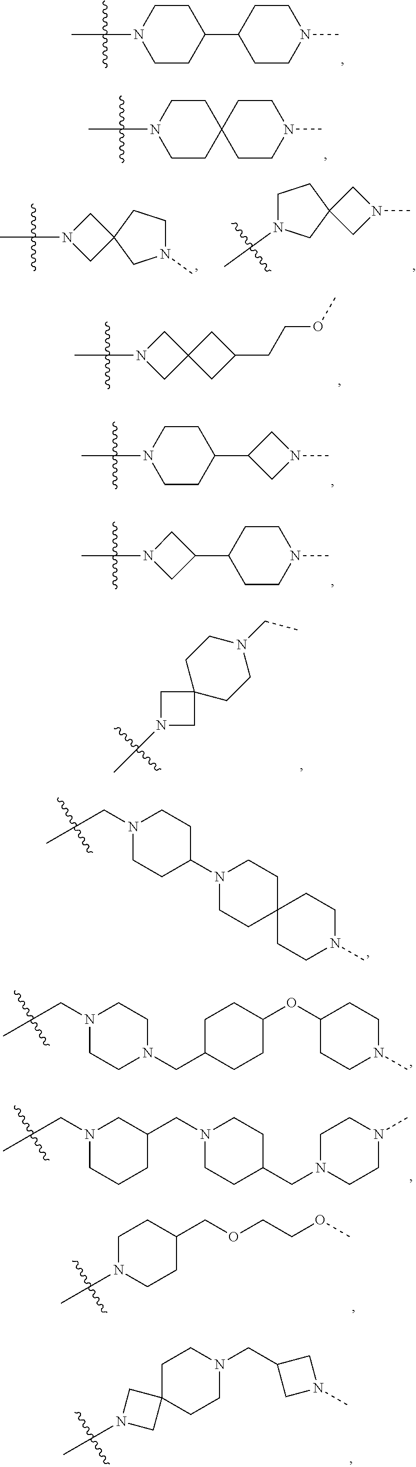 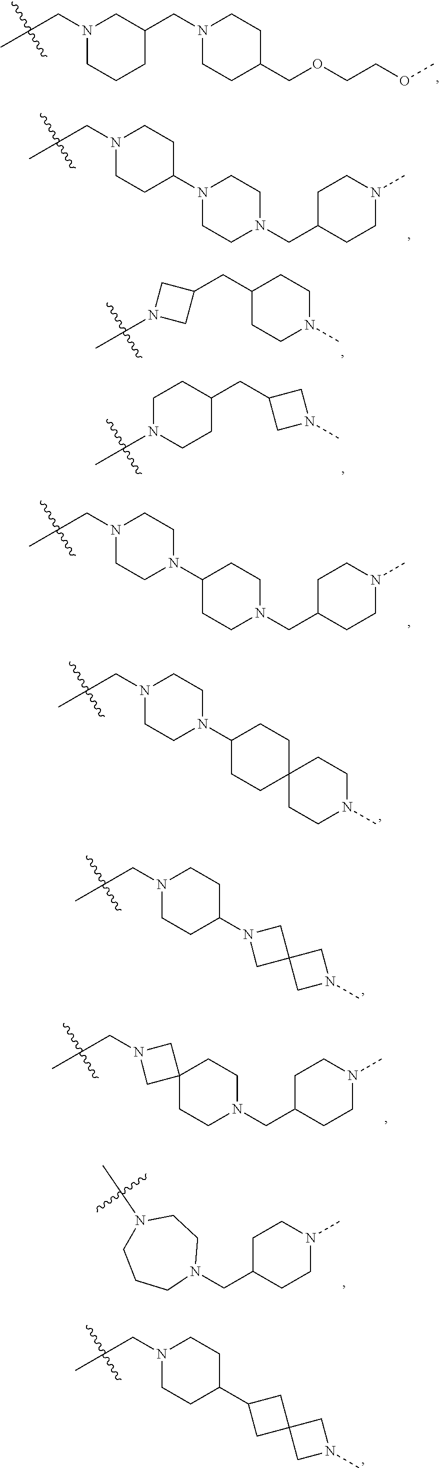 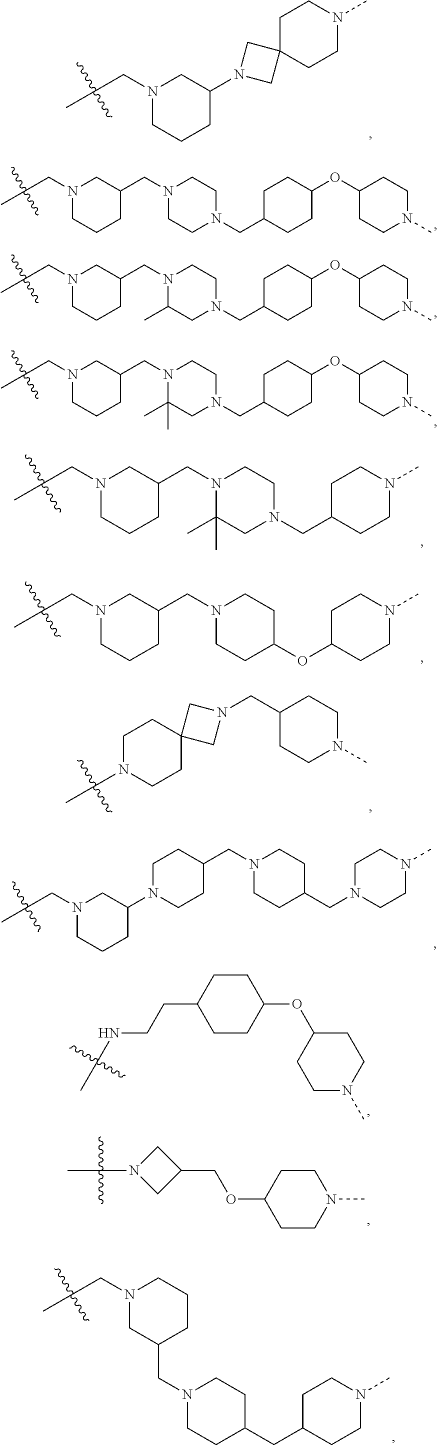 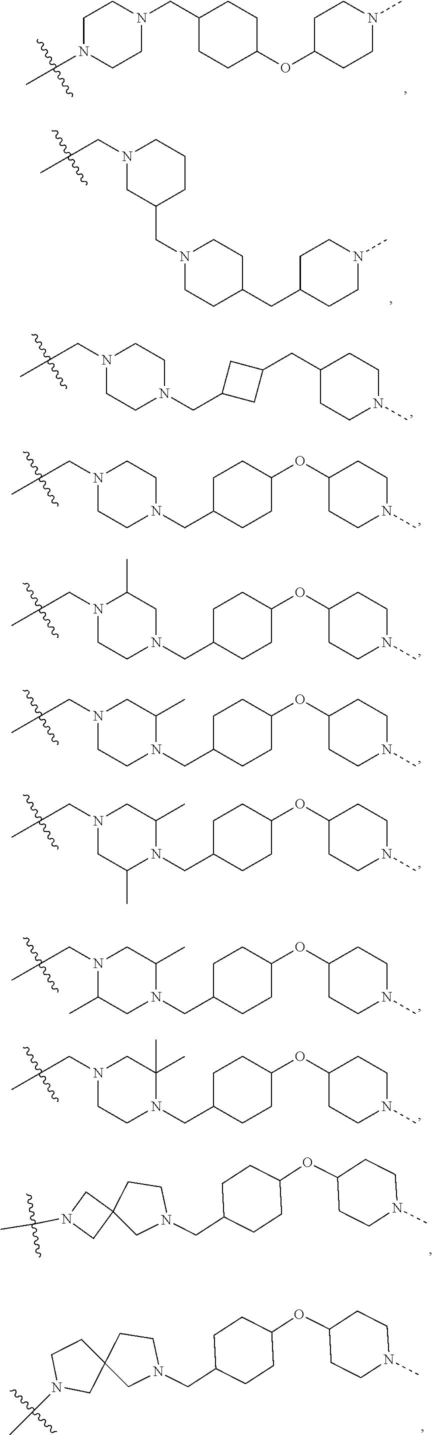 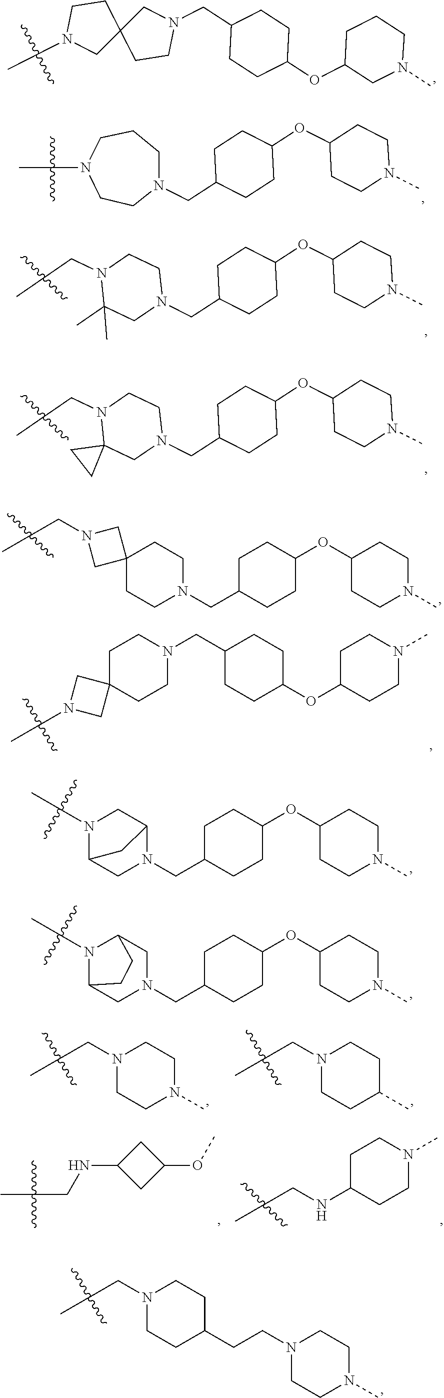 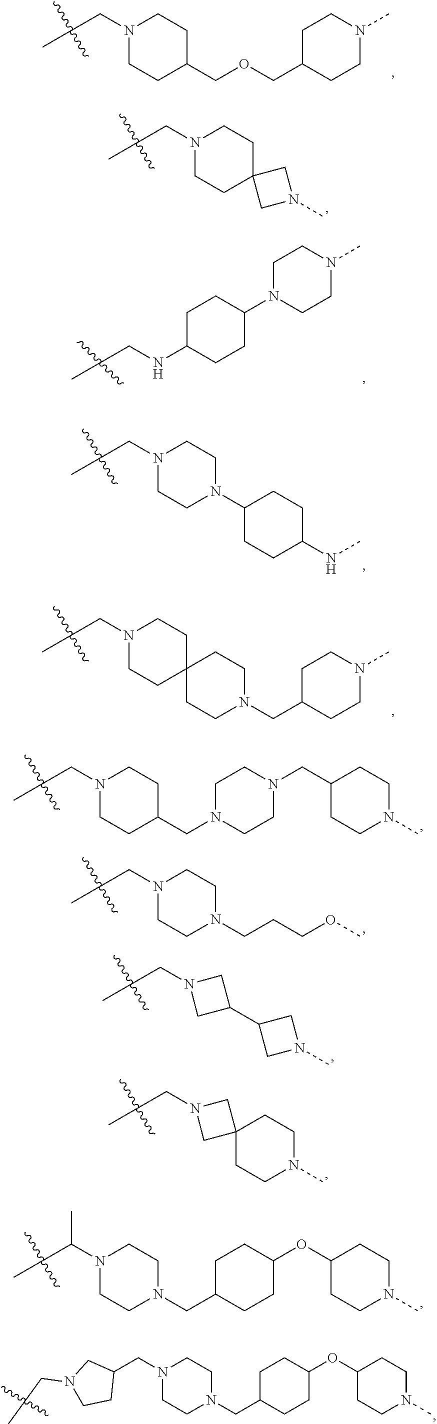 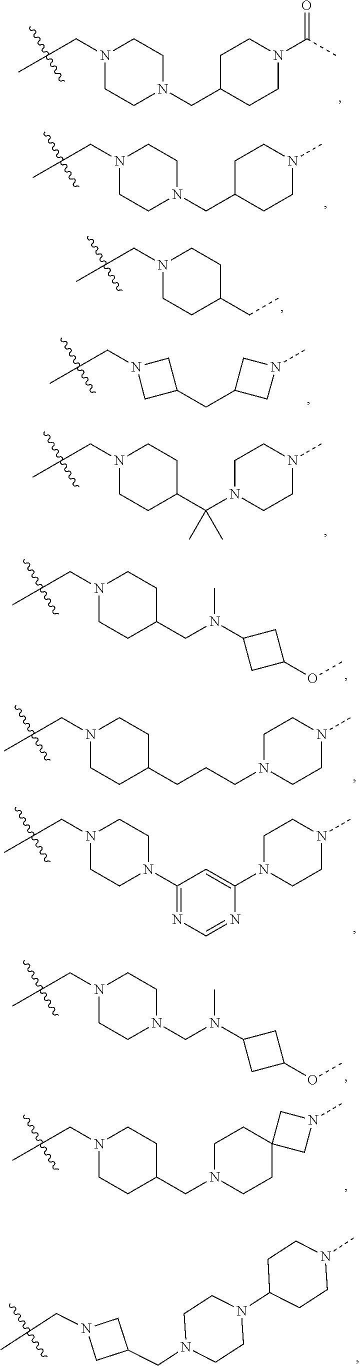 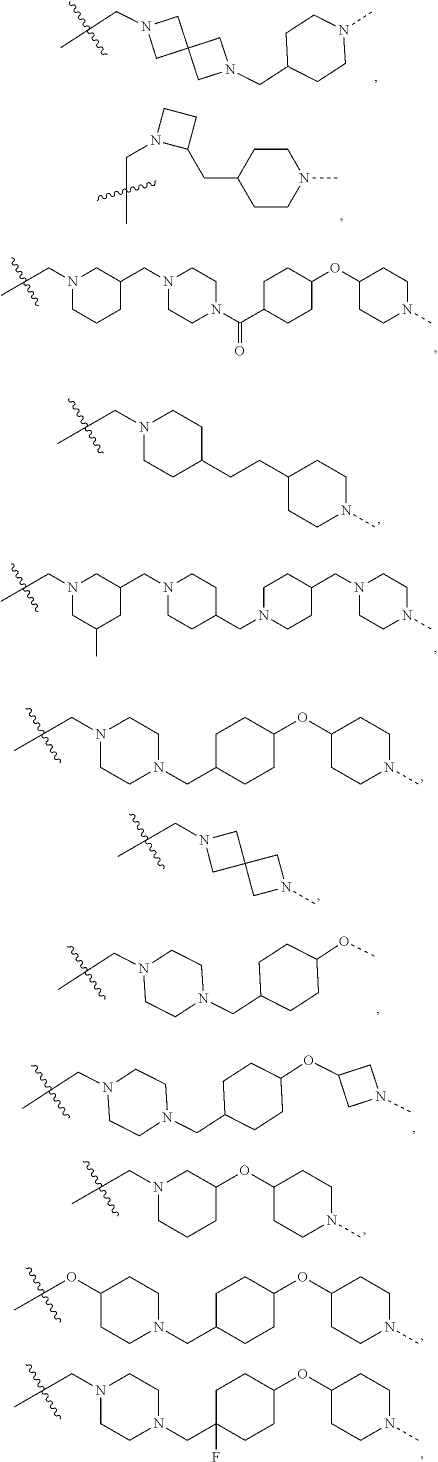 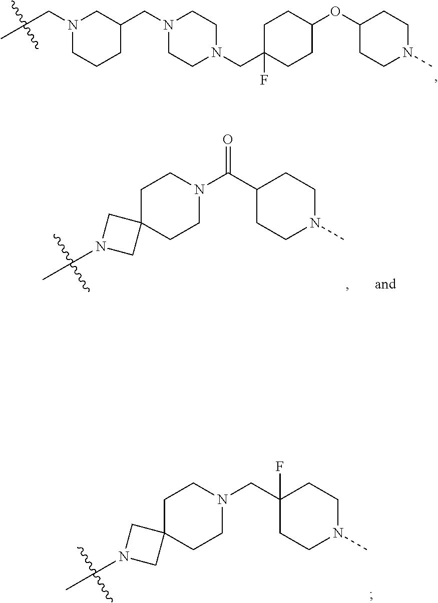
wherein the dashed line indicates the attachment point to E.

82. The compound of any one of claims 1 to 58, or a pharmaceutically acceptable salt thereof, wherein L is selected from 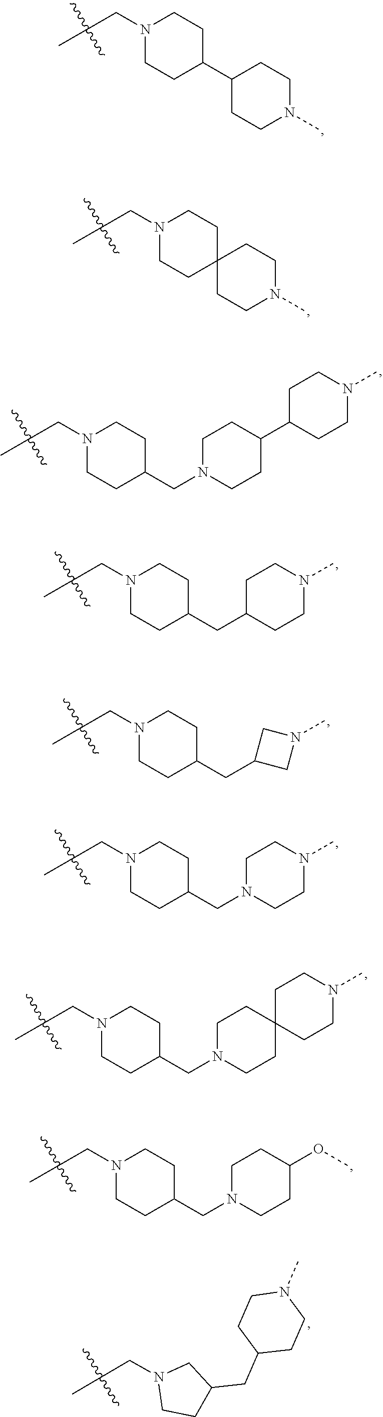 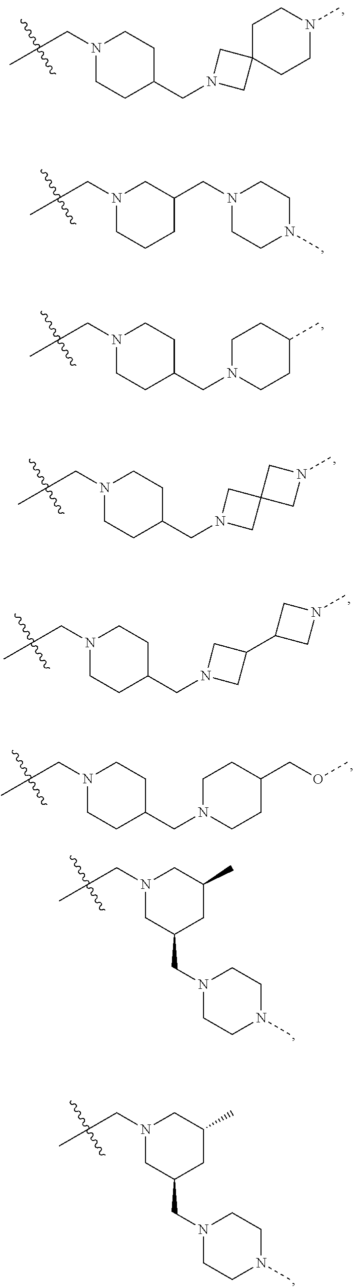 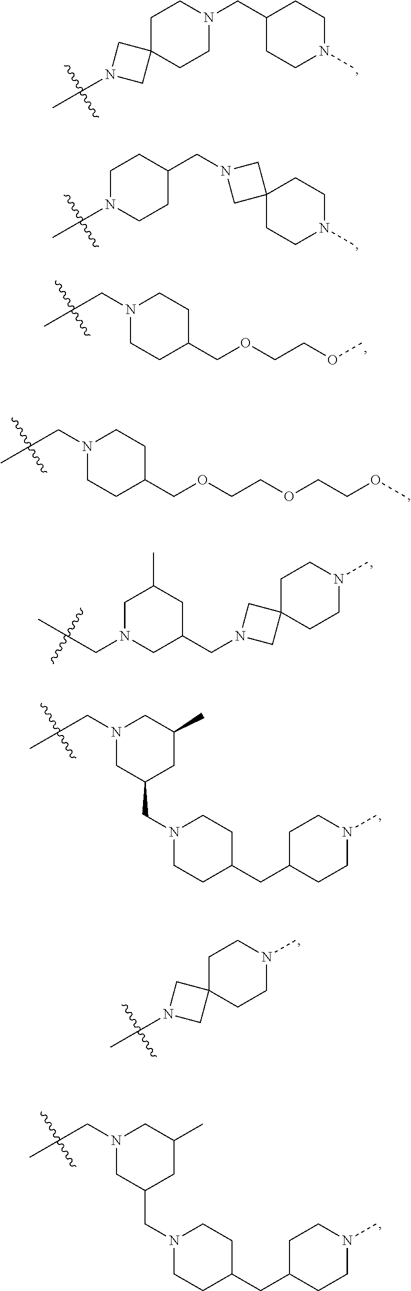 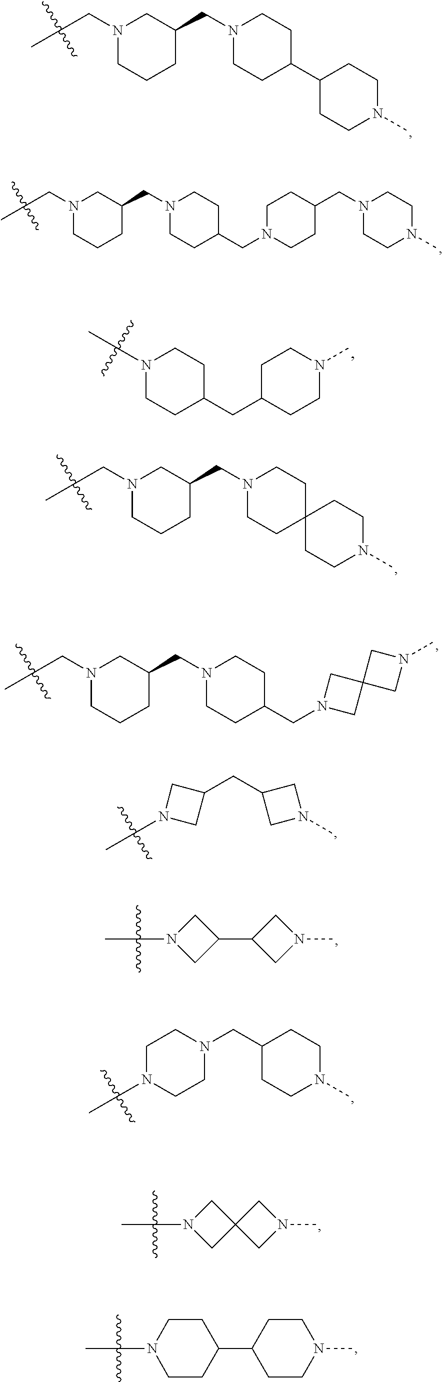 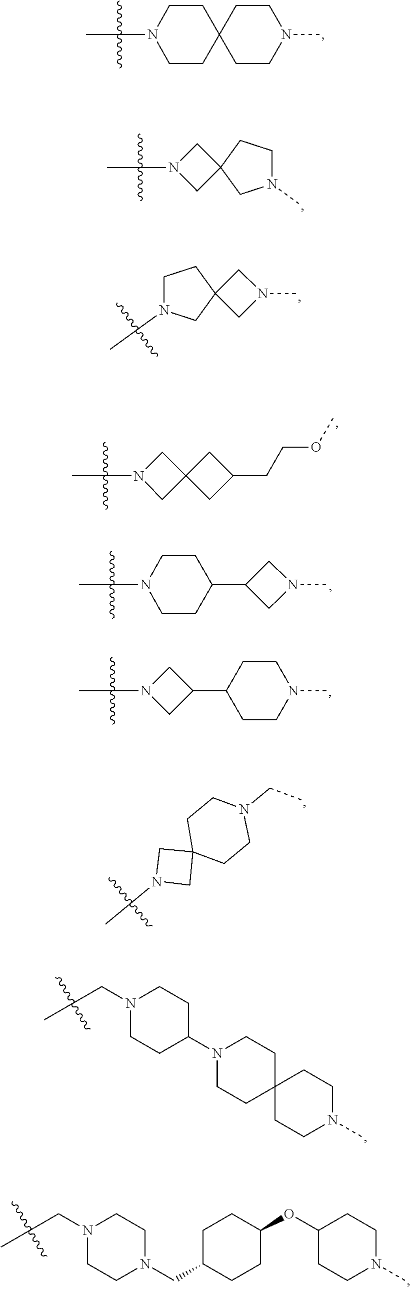 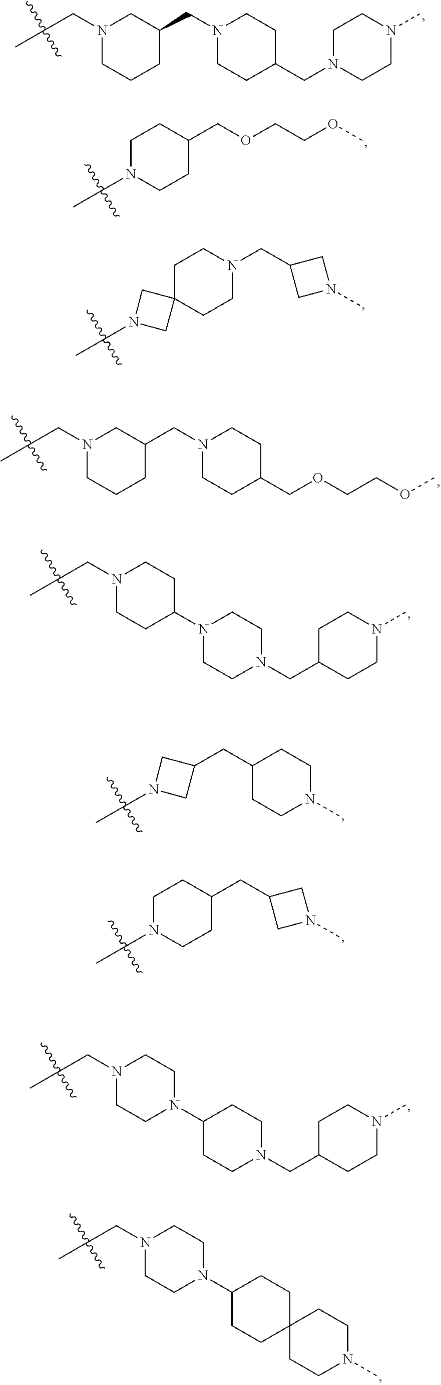 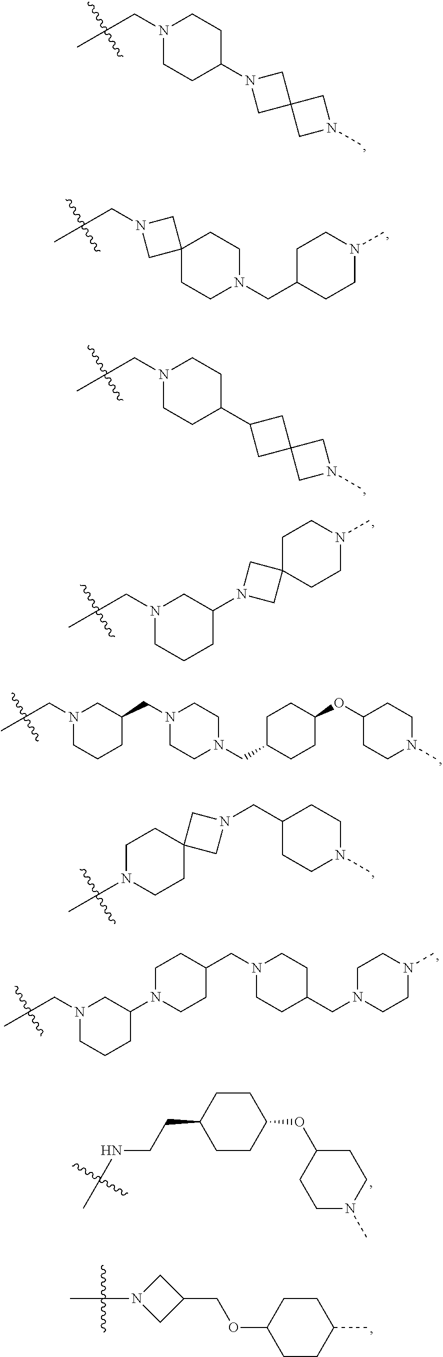 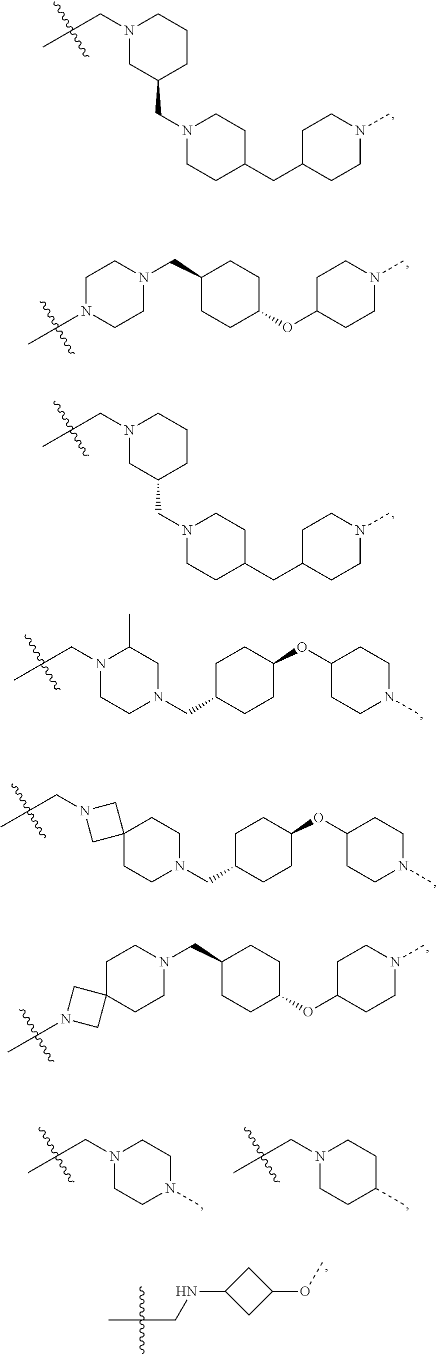 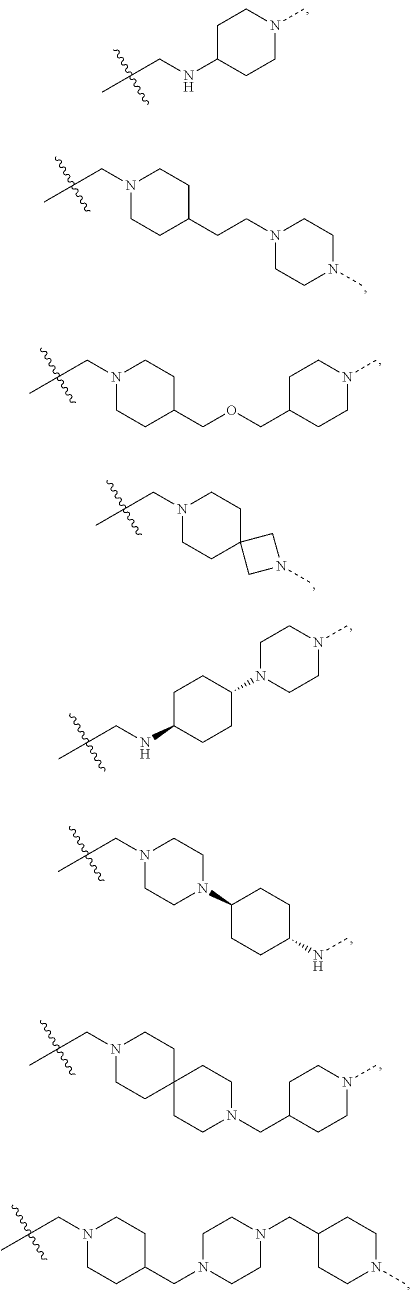 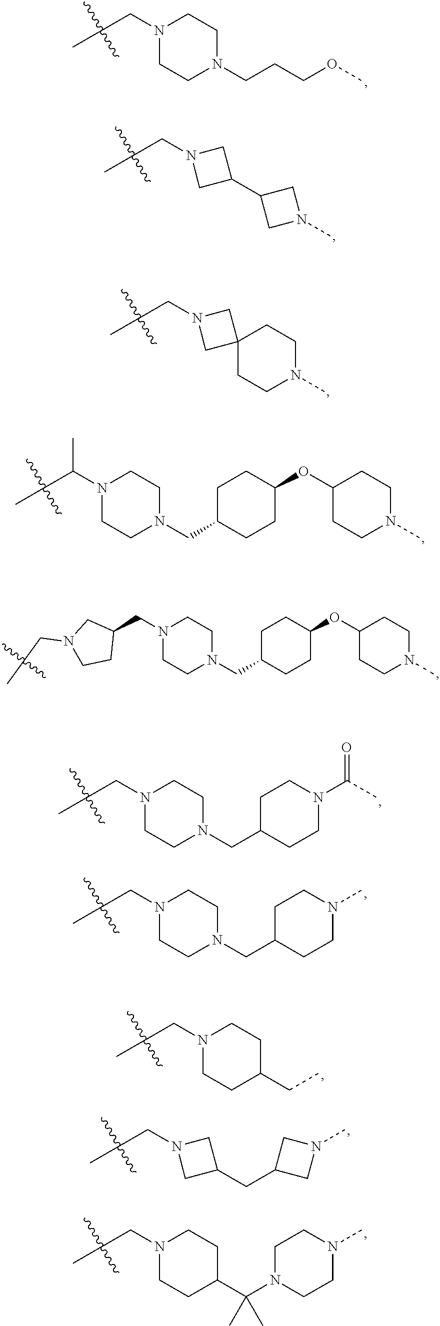 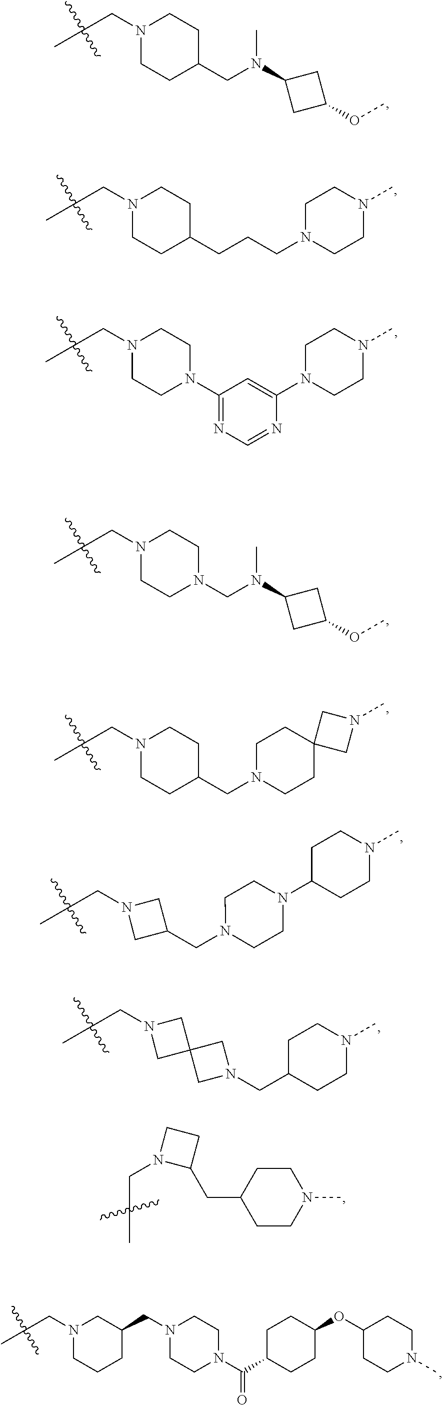 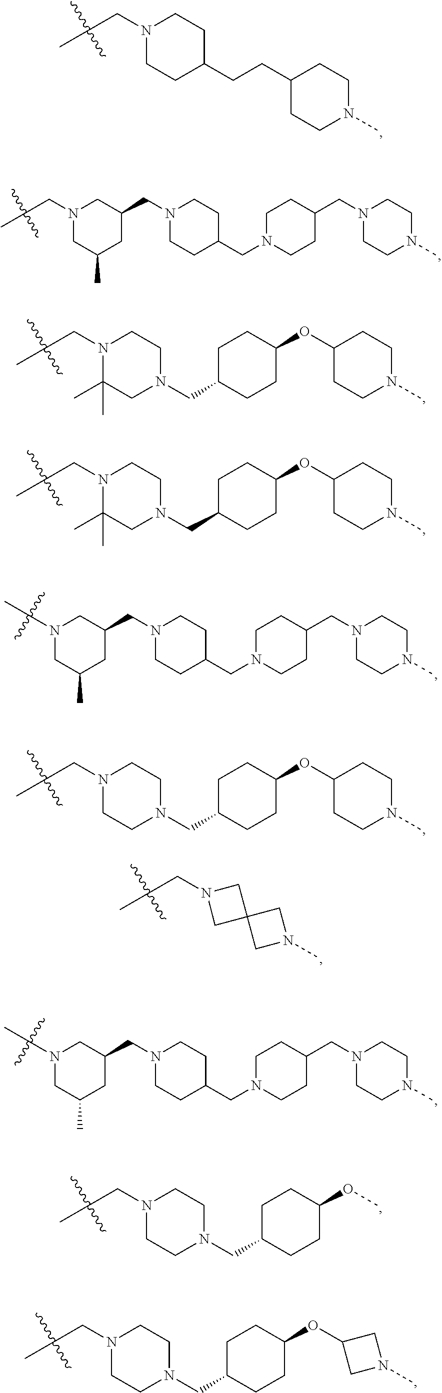 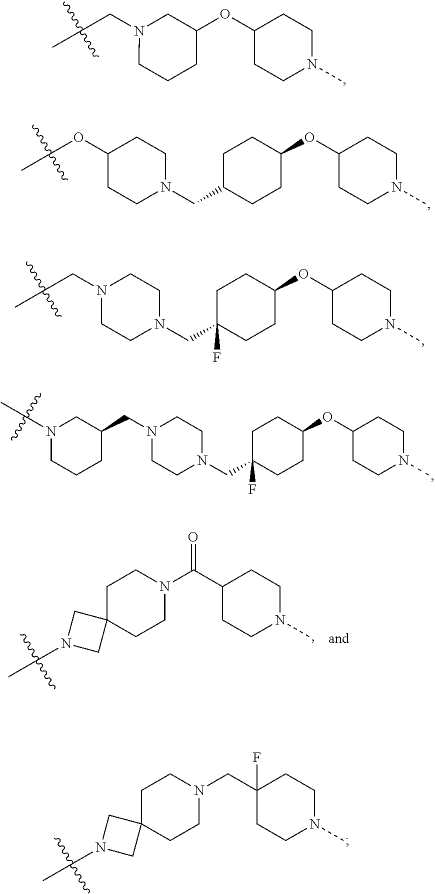
wherein the dashed line indicates the attachment point to E.

83. The compound of any one of claims 1 to 58, or a pharmaceutically acceptable salt thereof, wherein L is selected from 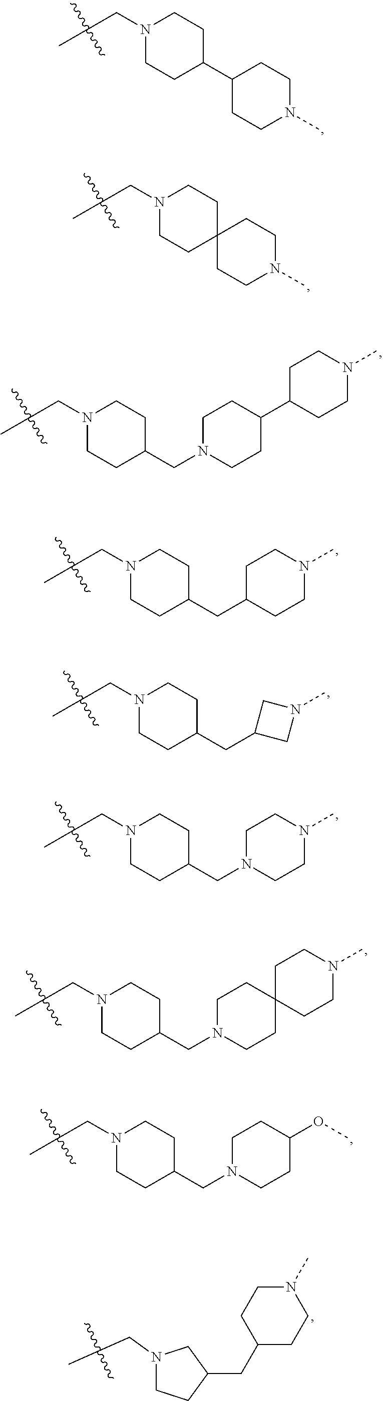 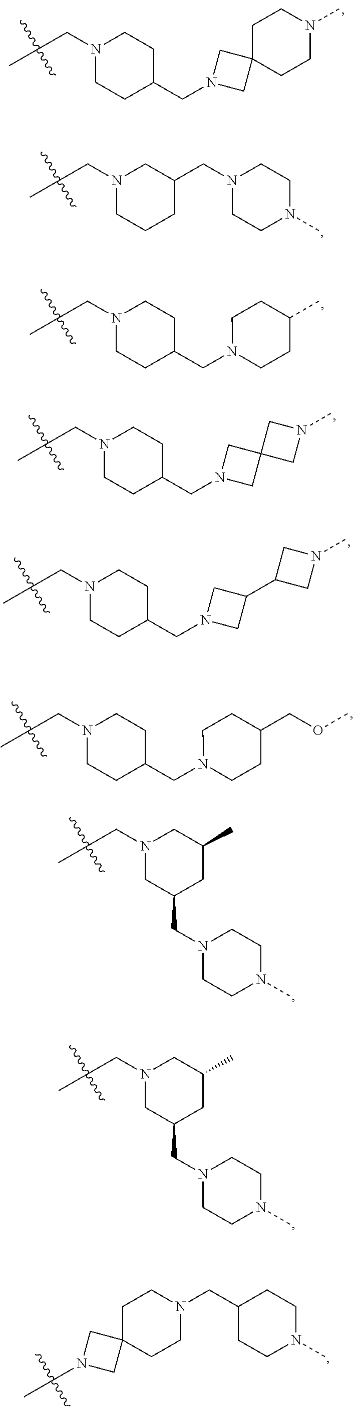 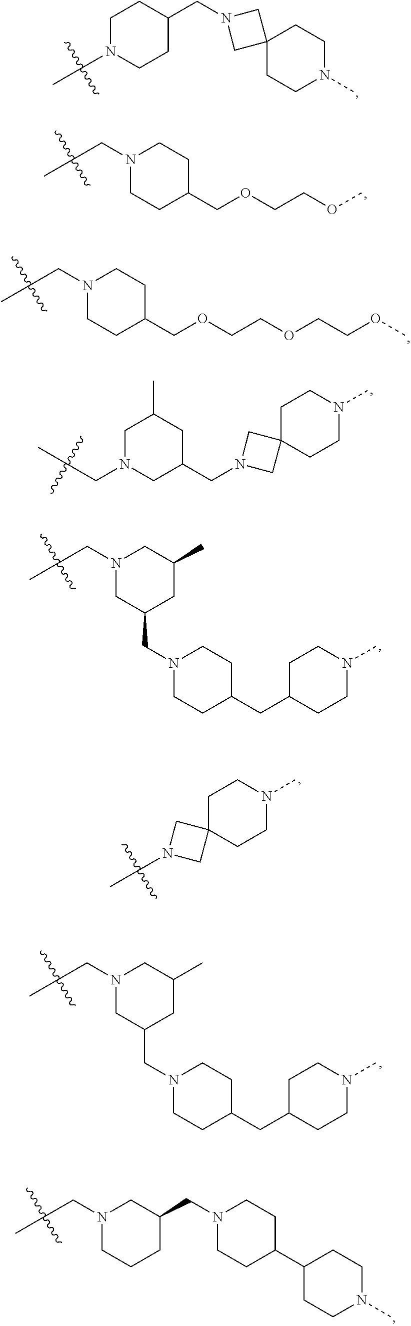 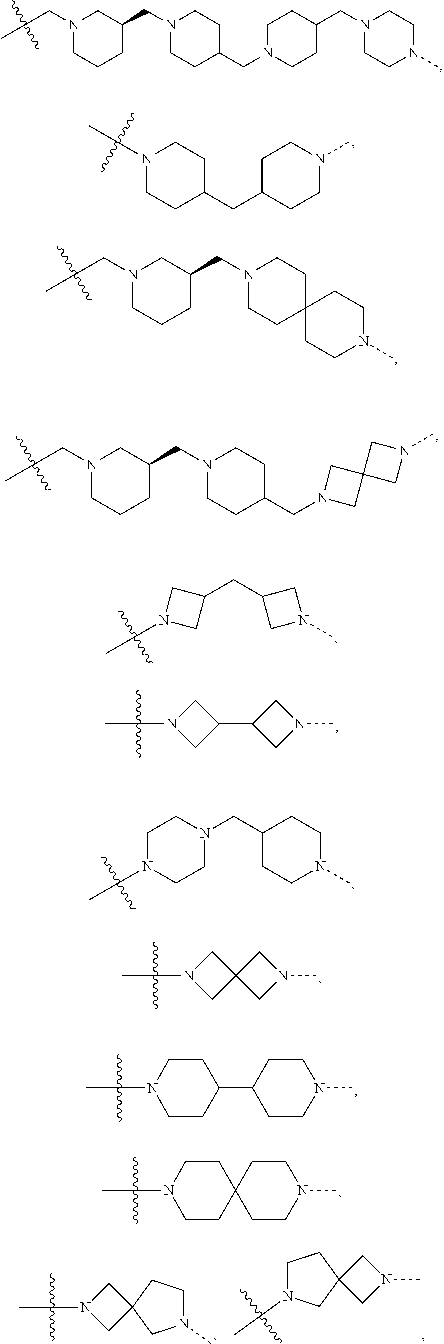 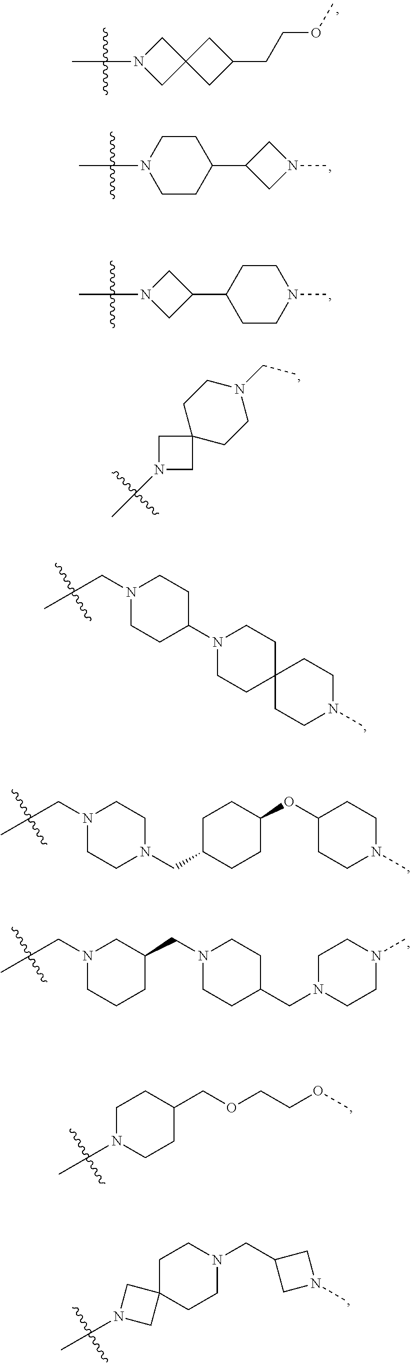 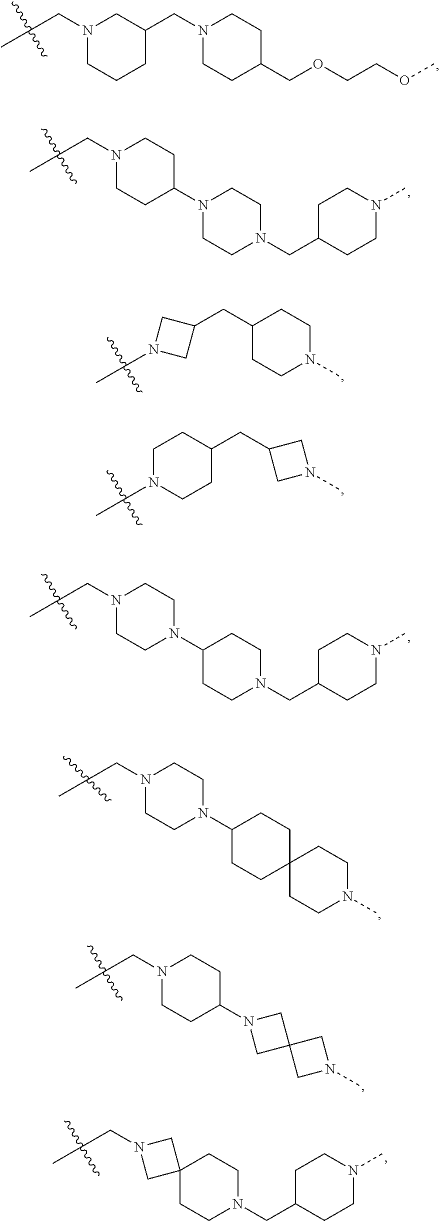 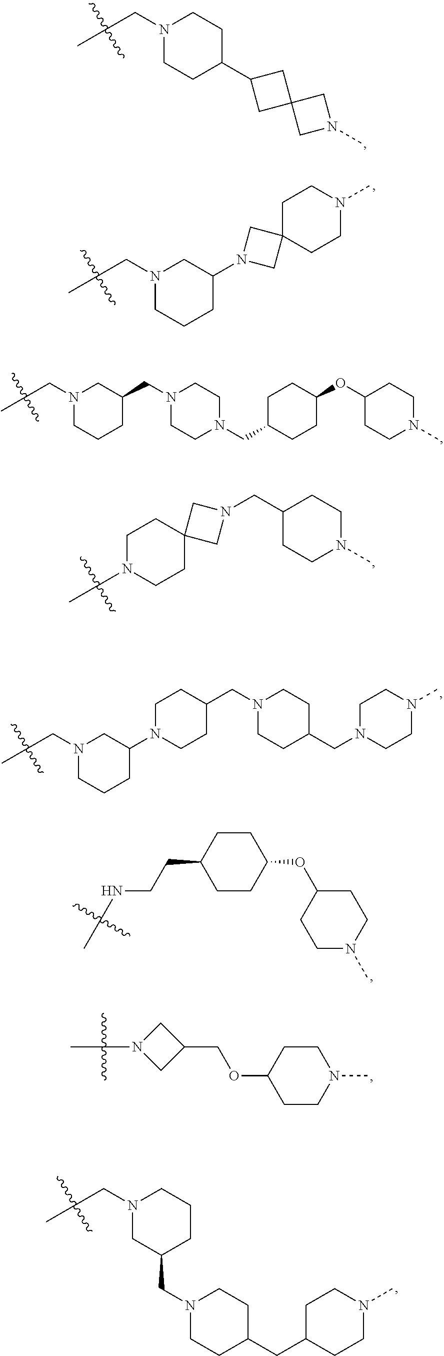 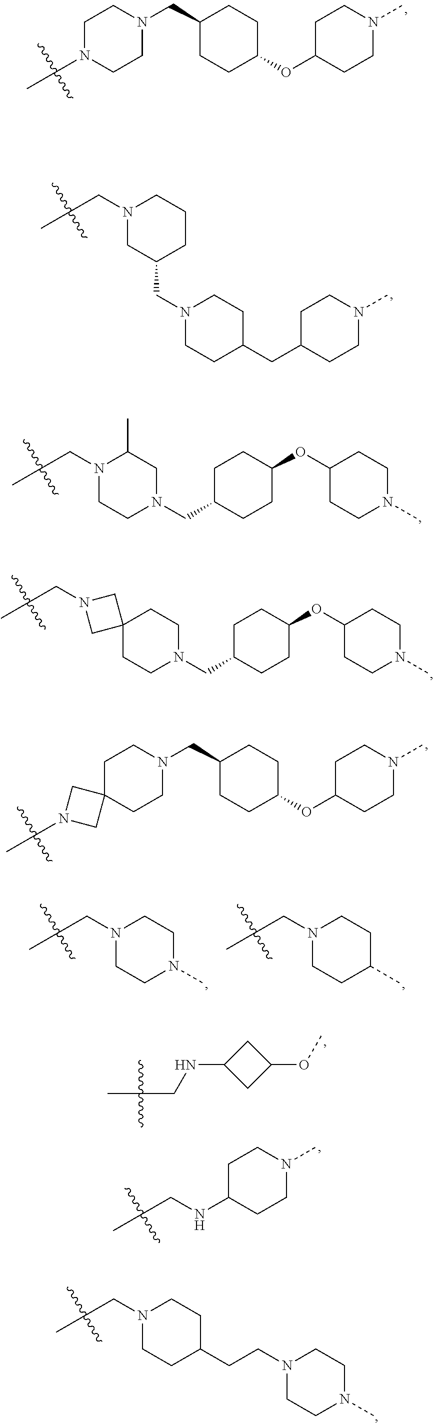 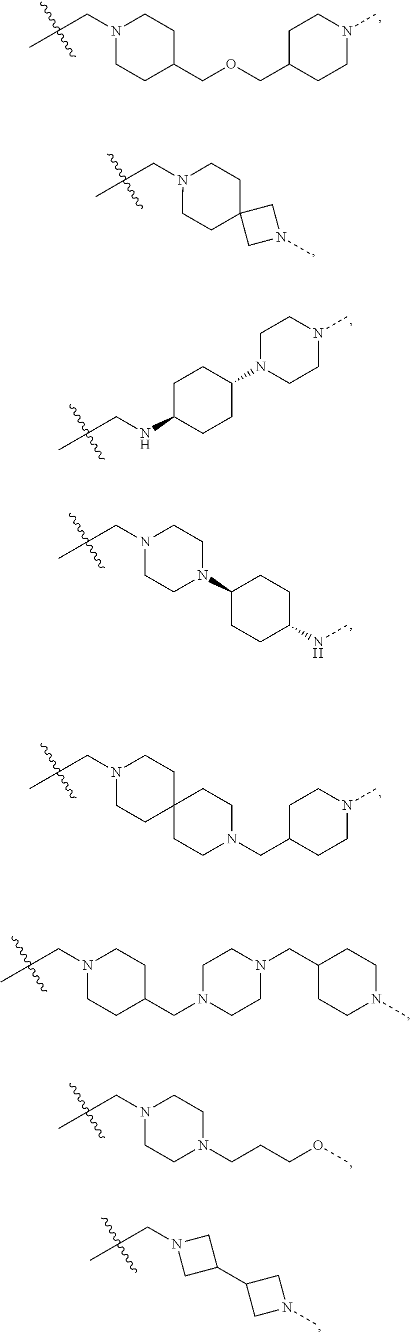 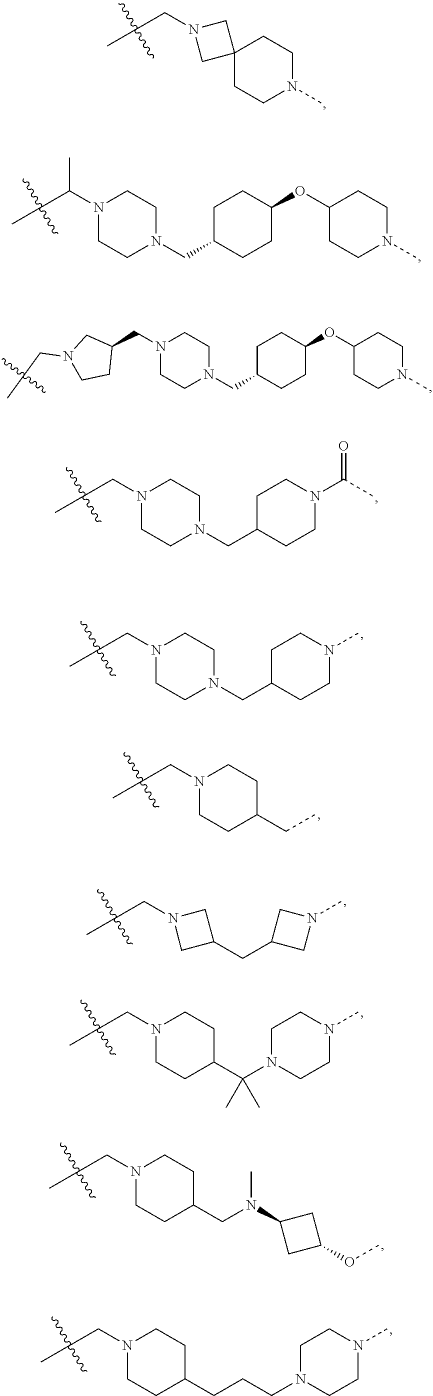 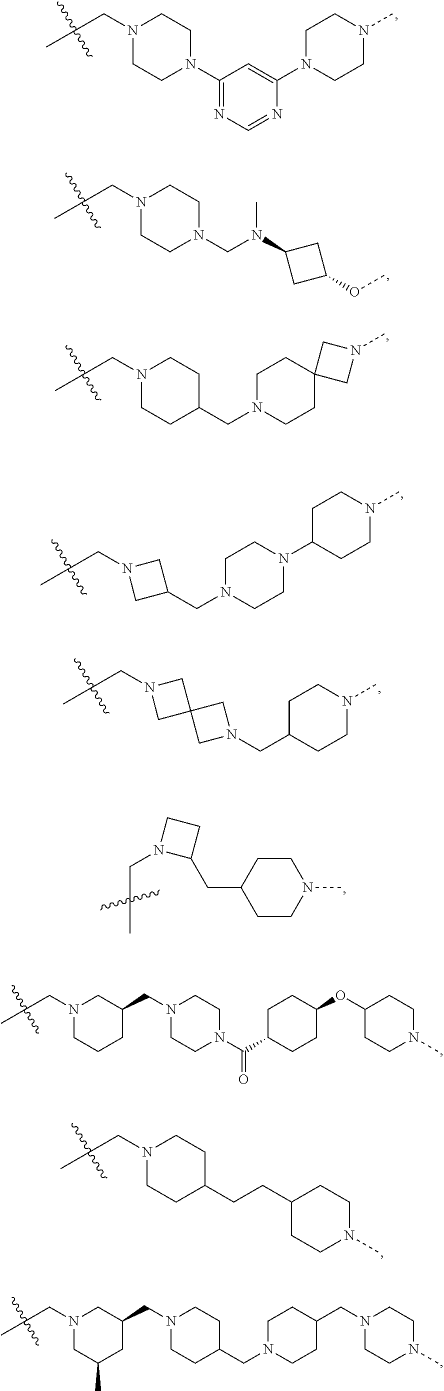 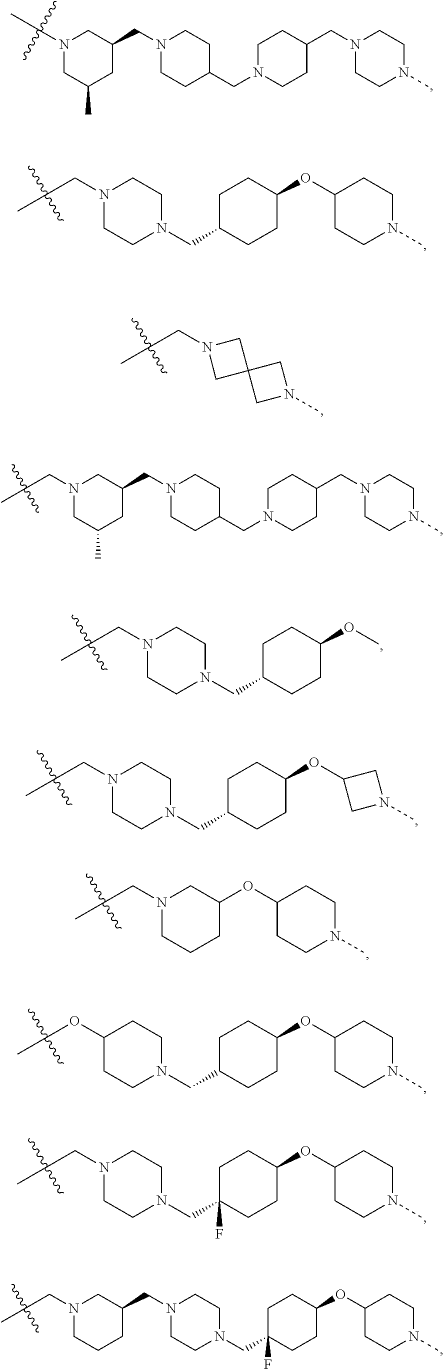 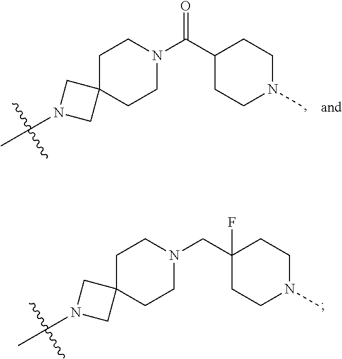
wherein the dashed line indicates the attachment point to E.

84. The compound of claim 1, wherein the compound is selected from any one of Examples 1 to 328; or a pharmaceutically acceptable salt thereof.

85. The compound of claim 1, wherein the compound is selected from any one of Examples 1 to 275; or a pharmaceutically acceptable salt thereof.

86. The compound of claim 1, wherein the compound is selected from any one of Examples 1 to 147; or a pharmaceutically acceptable salt thereof.

87. A pharmaceutical composition comprising a compound according to any of claims 1 to 72, or a pharmaceutically acceptable salt thereof; and a pharmaceutically acceptable carrier.

88. A method of treating a Cbl-B related condition in a subject in need thereof comprising administering to the subject a therapeutically effective amount of a compound according to any of claims 1 to 86, or a pharmaceutically acceptable salt thereof, or the pharmaceutical composition of claim 87.

89. The method of claim 88, wherein the Cbl-B related condition is cancer.

90. The method of claim 89, wherein the cancer is melanoma, lung cancer, head and neck cancer, prostate cancer, colorectal cancer, renal cancer, urothelial cancer, bladder cancer, hepatocellular carcinoma, pancreatic cancer, breast cancer, or hematology cancer.

91. The method of claim 89, wherein the cancer is non-small cell lung cancer, Squamous cell carcinoma of the head and neck, metastatic castration resistant prostate cancer, or MSS colorectal cancer.
